SEQUENCE LISTING (896 sequences)

SEQ ID NO: 1 - RNA, 30 nt, Artificial Sequence
  Synthetic
uacauaauuuagacguaagcaaugccauca

SEQ ID NO: 2 - RNA, 30 nt, Artificial Sequence
  Synthetic
acauaauuuagacguaagcaaugccaucac

SEQ ID NO: 3 - RNA, 30 nt, Artificial Sequence; identical to SEQ ID NO: 2
  Synthetic

SEQ ID NO: 4 - RNA, 32 nt, Artificial Sequence
  Synthetic
uacauaauuuagacguaagcaaugccaucacc

SEQ ID NO: 5 - RNA, 32 nt, Artificial Sequence
  Synthetic
uacauaauuuacacgaaagcaaugccaucacc

SEQ ID NO: 6 - RNA, 30 nt, Artificial Sequence; identical to SEQ ID NO: 2
  Synthetic

SEQ ID NO: 7 - RNA, 30 nt, Artificial Sequence
  Synthetic
acauaauuuacacgaaagcaaugccaucac

SEQ ID NO: 8 - RNA, 28 nt, Artificial Sequence
  Synthetic
auaauuuagacguaagcaaugccaucac

SEQ ID NO: 9 - RNA, 28 nt, Artificial Sequence
  Synthetic
auaauuuacacgaaagcaaugccaucac

SEQ ID NO: 10 - RNA, 27 nt, Artificial Sequence
  Synthetic
auaauuuagacguaagcaaugccauca

SEQ ID NO: 11 - RNA, 27 nt, Artificial Sequence
  Synthetic
auaauuuacacgaaagcaaugccauca

SEQ ID NO: 12 - RNA, 32 nt, Artificial Sequence; identical to SEQ ID NO: 5
  Synthetic

SEQ ID NO: 13 - RNA, 30 nt, Artificial Sequence; identical to SEQ ID NO: 2
  Synthetic

SEQ ID NO: 14 - RNA, 32 nt, Artificial Sequence; identical to SEQ ID NO: 5
  Synthetic

SEQ ID NO: 15 - RNA, 30 nt, Artificial Sequence; identical to SEQ ID NO: 2
  Synthetic

SEQ ID NO: 16 - RNA, 32 nt, Artificial Sequence; identical to SEQ ID NO: 5
  Synthetic

SEQ ID NO: 17 - RNA, 30 nt, Artificial Sequence; identical to SEQ ID NO: 2
  Synthetic

SEQ ID NO: 18 - RNA, 32 nt, Artificial Sequence; identical to SEQ ID NO: 4
  Synthetic

SEQ ID NO: 19 - RNA, 30 nt, Artificial Sequence; identical to SEQ ID NO: 2
  Synthetic

SEQ ID NO: 20 - RNA, 32 nt, Artificial Sequence; identical to SEQ ID NO: 4
  Synthetic

SEQ ID NO: 21 - RNA, 30 nt, Artificial Sequence; identical to SEQ ID NO: 2
  Synthetic

SEQ ID NO: 22 - RNA, 30 nt, Artificial Sequence
  Synthetic
acauaauuuagacguaagcaaugcuaucac

SEQ ID NO: 23 - RNA, 30 nt, Artificial Sequence; identical to SEQ ID NO: 22
  Synthetic

SEQ ID NO: 24 - RNA, 30 nt, Artificial Sequence; identical to SEQ ID NO: 22
  Synthetic

SEQ ID NO: 25 - RNA, 30 nt, Artificial Sequence; identical to SEQ ID NO: 22
  Synthetic

SEQ ID NO: 26 - RNA, 30 nt, Artificial Sequence; identical to SEQ ID NO: 22
  Synthetic

SEQ ID NO: 27 - RNA, 30 nt, Artificial Sequence; identical to SEQ ID NO: 2
  Synthetic

SEQ ID NO: 28 - RNA, 30 nt, Artificial Sequence; identical to SEQ ID NO: 2
  Synthetic

SEQ ID NO: 29 - RNA, 30 nt, Artificial Sequence; identical to SEQ ID NO: 2
  Synthetic

SEQ ID NO: 30 - RNA, 30 nt, Artificial Sequence; identical to SEQ ID NO: 2
  Synthetic

SEQ ID NO: 31 - RNA, 30 nt, Artificial Sequence; identical to SEQ ID NO: 2
  Synthetic

SEQ ID NO: 32 - RNA, 30 nt, Artificial Sequence; identical to SEQ ID NO: 2
  Synthetic

SEQ ID NO: 33 - RNA, 30 nt, Artificial Sequence; identical to SEQ ID NO: 2
  Synthetic

SEQ ID NO: 34 - RNA, 30 nt, Artificial Sequence; identical to SEQ ID NO: 2
  Synthetic

SEQ ID NO: 35 - RNA, 30 nt, Artificial Sequence; identical to SEQ ID NO: 2
  Synthetic

SEQ ID NO: 36 - RNA, 30 nt, Artificial Sequence; identical to SEQ ID NO: 2
  Synthetic

SEQ ID NO: 37 - RNA, 30 nt, Artificial Sequence; identical to SEQ ID NO: 2
  Synthetic

SEQ ID NO: 38 - RNA, 30 nt, Artificial Sequence; identical to SEQ ID NO: 2
  Synthetic

SEQ ID NO: 39 - RNA, 30 nt, Artificial Sequence; identical to SEQ ID NO: 2
  Synthetic

SEQ ID NO: 40 - RNA, 30 nt, Artificial Sequence; identical to SEQ ID NO: 2
  Synthetic

SEQ ID NO: 41 - RNA, 30 nt, Artificial Sequence; identical to SEQ ID NO: 2
  Synthetic

SEQ ID NO: 42 - RNA, 30 nt, Artificial Sequence; identical to SEQ ID NO: 2
  Synthetic

SEQ ID NO: 43 - RNA, 30 nt, Artificial Sequence; identical to SEQ ID NO: 2
  Synthetic

SEQ ID NO: 44 - RNA, 30 nt, Artificial Sequence; identical to SEQ ID NO: 2
  Synthetic

SEQ ID NO: 45 - RNA, 30 nt, Artificial Sequence; identical to SEQ ID NO: 2
  Synthetic

SEQ ID NO: 46 - RNA, 30 nt, Artificial Sequence; identical to SEQ ID NO: 2
  Synthetic

SEQ ID NO: 47 - RNA, 30 nt, Artificial Sequence; identical to SEQ ID NO: 2
  Synthetic

SEQ ID NO: 48 - RNA, 30 nt, Artificial Sequence
  Synthetic
acauaauuuacacaaaggcaaugccaucac

SEQ ID NO: 49 - RNA, 30 nt, Artificial Sequence; identical to SEQ ID NO: 48
  Synthetic

SEQ ID NO: 50 - RNA, 30 nt, Artificial Sequence; identical to SEQ ID NO: 7
  Synthetic

SEQ ID NO: 51 - RNA, 30 nt, Artificial Sequence; identical to SEQ ID NO: 7
  Synthetic

SEQ ID NO: 52 - RNA, 30 nt, Artificial Sequence; identical to SEQ ID NO: 7
  Synthetic

SEQ ID NO: 53 - RNA, 30 nt, Artificial Sequence; identical to SEQ ID NO: 7
  Synthetic

SEQ ID NO: 54 - RNA, 30 nt, Artificial Sequence; identical to SEQ ID NO: 7
  Synthetic

SEQ ID NO: 55 - RNA, 30 nt, Artificial Sequence; identical to SEQ ID NO: 7
  Synthetic

SEQ ID NO: 56 - RNA, 30 nt, Artificial Sequence; identical to SEQ ID NO: 7
  Synthetic

SEQ ID NO: 57 - RNA, 30 nt, Artificial Sequence; identical to SEQ ID NO: 7
  Synthetic

SEQ ID NO: 58 - RNA, 30 nt, Artificial Sequence; identical to SEQ ID NO: 7
  Synthetic

SEQ ID NO: 59 - RNA, 30 nt, Artificial Sequence; identical to SEQ ID NO: 7
  Synthetic

SEQ ID NO: 60 - RNA, 30 nt, Artificial Sequence; identical to SEQ ID NO: 7
  Synthetic

SEQ ID NO: 61 - RNA, 30 nt, Artificial Sequence; identical to SEQ ID NO: 7
  Synthetic

SEQ ID NO: 62 - RNA, 30 nt, Artificial Sequence; identical to SEQ ID NO: 7
  Synthetic

SEQ ID NO: 63 - RNA, 32 nt, Artificial Sequence
  Synthetic
ucucuuuccauggaagguucuaaaccauccug

SEQ ID NO: 64 - RNA, 32 nt, Artificial Sequence; identical to SEQ ID NO: 63
  Synthetic

SEQ ID NO: 65 - RNA, 32 nt, Artificial Sequence; identical to SEQ ID NO: 63
  Synthetic

SEQ ID NO: 66 - RNA, 32 nt, Artificial Sequence; identical to SEQ ID NO: 63
  Synthetic

SEQ ID NO: 67 - RNA, 32 nt, Artificial Sequence; identical to SEQ ID NO: 63
  Synthetic

SEQ ID NO: 68 - RNA, 32 nt, Artificial Sequence; identical to SEQ ID NO: 63
  Synthetic

SEQ ID NO: 69 - RNA, 32 nt, Artificial Sequence; identical to SEQ ID NO: 63
  Synthetic

SEQ ID NO: 70 - RNA, 32 nt, Artificial Sequence; identical to SEQ ID NO: 63
  Synthetic

SEQ ID NO: 71 - RNA, 32 nt, Artificial Sequence; identical to SEQ ID NO: 63
  Synthetic

SEQ ID NO: 72 - RNA, 32 nt, Artificial Sequence; identical to SEQ ID NO: 63
  Synthetic

SEQ ID NO: 73 - RNA, 32 nt, Artificial Sequence; identical to SEQ ID NO: 63
  Synthetic

SEQ ID NO: 74 - RNA, 32 nt, Artificial Sequence; identical to SEQ ID NO: 63
  Synthetic

SEQ ID NO: 75 - RNA, 32 nt, Artificial Sequence; identical to SEQ ID NO: 63
  Synthetic

SEQ ID NO: 76 - RNA, 32 nt, Artificial Sequence; identical to SEQ ID NO: 63
  Synthetic

SEQ ID NO: 77 - RNA, 32 nt, Artificial Sequence; identical to SEQ ID NO: 63
  Synthetic

SEQ ID NO: 78 - RNA, 32 nt, Artificial Sequence; identical to SEQ ID NO: 63
  Synthetic

SEQ ID NO: 79 - RNA, 32 nt, Artificial Sequence; identical to SEQ ID NO: 63
  Synthetic

SEQ ID NO: 80 - RNA, 32 nt, Artificial Sequence; identical to SEQ ID NO: 63
  Synthetic

SEQ ID NO: 81 - RNA, 32 nt, Artificial Sequence; identical to SEQ ID NO: 63
  Synthetic

SEQ ID NO: 82 - RNA, 32 nt, Artificial Sequence; identical to SEQ ID NO: 63
  Synthetic

SEQ ID NO: 83 - RNA, 32 nt, Artificial Sequence; identical to SEQ ID NO: 63
  Synthetic

SEQ ID NO: 84 - RNA, 32 nt, Artificial Sequence; identical to SEQ ID NO: 63
  Synthetic

SEQ ID NO: 85 - RNA, 32 nt, Artificial Sequence; identical to SEQ ID NO: 63
  Synthetic

SEQ ID NO: 86 - RNA, 32 nt, Artificial Sequence; identical to SEQ ID NO: 63
  Synthetic

SEQ ID NO: 87 - RNA, 32 nt, Artificial Sequence; identical to SEQ ID NO: 63
  Synthetic

SEQ ID NO: 88 - RNA, 32 nt, Artificial Sequence; identical to SEQ ID NO: 63
  Synthetic

SEQ ID NO: 89 - RNA, 32 nt, Artificial Sequence; identical to SEQ ID NO: 63
  Synthetic

SEQ ID NO: 90 - RNA, 32 nt, Artificial Sequence; identical to SEQ ID NO: 63
  Synthetic

SEQ ID NO: 91 - RNA, 32 nt, Artificial Sequence; identical to SEQ ID NO: 63
  Synthetic

SEQ ID NO: 92 - RNA, 32 nt, Artificial Sequence; identical to SEQ ID NO: 63
  Synthetic

SEQ ID NO: 93 - RNA, 32 nt, Artificial Sequence; identical to SEQ ID NO: 63
  Synthetic

SEQ ID NO: 94 - RNA, 32 nt, Artificial Sequence; identical to SEQ ID NO: 63
  Synthetic

SEQ ID NO: 95 - RNA, 32 nt, Artificial Sequence; identical to SEQ ID NO: 63
  Synthetic

SEQ ID NO: 96 - RNA, 32 nt, Artificial Sequence; identical to SEQ ID NO: 63
  Synthetic

SEQ ID NO: 97 - RNA, 32 nt, Artificial Sequence; identical to SEQ ID NO: 63
  Synthetic

SEQ ID NO: 98 - RNA, 32 nt, Artificial Sequence; identical to SEQ ID NO: 63
  Synthetic

SEQ ID NO: 99 - RNA, 32 nt, Artificial Sequence; identical to SEQ ID NO: 63
  Synthetic

SEQ ID NO: 100 - RNA, 32 nt, Artificial Sequence; identical to SEQ ID NO: 63
  Synthetic

SEQ ID NO: 101 - RNA, 32 nt, Artificial Sequence; identical to SEQ ID NO: 63
  Synthetic

SEQ ID NO: 102 - RNA, 32 nt, Artificial Sequence; identical to SEQ ID NO: 63
  Synthetic

SEQ ID NO: 103 - RNA, 32 nt, Artificial Sequence; identical to SEQ ID NO: 63
  Synthetic

SEQ ID NO: 104 - RNA, 32 nt, Artificial Sequence; identical to SEQ ID NO: 63
  Synthetic

SEQ ID NO: 105 - RNA, 32 nt, Artificial Sequence; identical to SEQ ID NO: 63
  Synthetic

SEQ ID NO: 106 - RNA, 32 nt, Artificial Sequence; identical to SEQ ID NO: 63
  Synthetic

SEQ ID NO: 107 - RNA, 32 nt, Artificial Sequence; identical to SEQ ID NO: 63
  Synthetic

SEQ ID NO: 108 - RNA, 32 nt, Artificial Sequence; identical to SEQ ID NO: 63
  Synthetic

SEQ ID NO: 109 - RNA, 32 nt, Artificial Sequence; identical to SEQ ID NO: 63
  Synthetic

SEQ ID NO: 110 - RNA, 32 nt, Artificial Sequence; identical to SEQ ID NO: 63
  Synthetic

SEQ ID NO: 111 - RNA, 32 nt, Artificial Sequence; identical to SEQ ID NO: 63
  Synthetic

SEQ ID NO: 112 - RNA, 32 nt, Artificial Sequence; identical to SEQ ID NO: 63
  Synthetic

SEQ ID NO: 113 - RNA, 32 nt, Artificial Sequence; identical to SEQ ID NO: 63
  Synthetic

SEQ ID NO: 114 - RNA, 32 nt, Artificial Sequence; identical to SEQ ID NO: 63
  Synthetic

SEQ ID NO: 115 - RNA, 32 nt, Artificial Sequence; identical to SEQ ID NO: 63
  Synthetic

SEQ ID NO: 116 - RNA, 32 nt, Artificial Sequence; identical to SEQ ID NO: 63
  Synthetic

SEQ ID NO: 117 - RNA, 32 nt, Artificial Sequence; identical to SEQ ID NO: 63
  Synthetic

SEQ ID NO: 118 - RNA, 32 nt, Artificial Sequence; identical to SEQ ID NO: 63
  Synthetic

SEQ ID NO: 119 - RNA, 32 nt, Artificial Sequence; identical to SEQ ID NO: 63
  Synthetic

SEQ ID NO: 120 - RNA, 32 nt, Artificial Sequence; identical to SEQ ID NO: 63
  Synthetic

SEQ ID NO: 121 - RNA, 32 nt, Artificial Sequence; identical to SEQ ID NO: 63
  Synthetic

SEQ ID NO: 122 - RNA, 32 nt, Artificial Sequence; identical to SEQ ID NO: 63
  Synthetic

SEQ ID NO: 123 - RNA, 32 nt, Artificial Sequence; identical to SEQ ID NO: 63
  Synthetic

SEQ ID NO: 124 - RNA, 32 nt, Artificial Sequence; identical to SEQ ID NO: 63
  Synthetic

SEQ ID NO: 125 - RNA, 32 nt, Artificial Sequence; identical to SEQ ID NO: 63
  Synthetic

SEQ ID NO: 126 - RNA, 32 nt, Artificial Sequence; identical to SEQ ID NO: 63
  Synthetic

SEQ ID NO: 127 - RNA, 32 nt, Artificial Sequence; identical to SEQ ID NO: 63
  Synthetic

SEQ ID NO: 128 - RNA, 32 nt, Artificial Sequence; identical to SEQ ID NO: 63
  Synthetic

SEQ ID NO: 129 - RNA, 32 nt, Artificial Sequence; identical to SEQ ID NO: 63
  Synthetic

SEQ ID NO: 130 - RNA, 32 nt, Artificial Sequence; identical to SEQ ID NO: 63
  Synthetic

SEQ ID NO: 131 - RNA, 32 nt, Artificial Sequence; identical to SEQ ID NO: 63
  Synthetic

SEQ ID NO: 132 - RNA, 32 nt, Artificial Sequence; identical to SEQ ID NO: 63
  Synthetic

SEQ ID NO: 133 - RNA, 32 nt, Artificial Sequence; identical to SEQ ID NO: 63
  Synthetic

SEQ ID NO: 134 - RNA, 32 nt, Artificial Sequence; identical to SEQ ID NO: 63
  Synthetic

SEQ ID NO: 135 - RNA, 31 nt, Artificial Sequence
  Synthetic
ucucuuuccauggaagguucuaaaccauccu

SEQ ID NO: 136 - RNA, 30 nt, Artificial Sequence
  Synthetic
ucucuuuccauggaagguucuaaaccaucc

SEQ ID NO: 137 - RNA, 29 nt, Artificial Sequence
  Synthetic
ucucuuuccauggaagguucuaaaccauc

SEQ ID NO: 138 - RNA, 31 nt, Artificial Sequence
  Synthetic
cucuuuccauggaagguucuaaaccauccug

SEQ ID NO: 139 - RNA, 30 nt, Artificial Sequence
  Synthetic
cucuuuccauggaagguucuaaaccauccu

SEQ ID NO: 140 - RNA, 29 nt, Artificial Sequence
  Synthetic
cucuuuccauggaagguucuaaaccaucc

SEQ ID NO: 141 - RNA, 28 nt, Artificial Sequence
  Synthetic
cucuuuccauggaagguucuaaaccauc

SEQ ID NO: 142 - RNA, 30 nt, Artificial Sequence
  Synthetic
ucuuuccauggaagguucuaaaccauccug

SEQ ID NO: 143 - RNA, 29 nt, Artificial Sequence
  Synthetic
ucuuuccauggaagguucuaaaccauccu

SEQ ID NO: 144 - RNA, 28 nt, Artificial Sequence
  Synthetic
ucuuuccauggaagguucuaaaccaucc

SEQ ID NO: 145 - RNA, 27 nt, Artificial Sequence
  Synthetic
ucuuuccauggaagguucuaaaccauc

SEQ ID NO: 146 - RNA, 29 nt, Artificial Sequence
  Synthetic
cuuuccauggaagguucuaaaccauccug

SEQ ID NO: 147 - RNA, 28 nt, Artificial Sequence
  Synthetic
cuuuccauggaagguucuaaaccauccu

SEQ ID NO: 148 - RNA, 27 nt, Artificial Sequence
  Synthetic
cuuuccauggaagguucuaaaccaucc

SEQ ID NO: 149 - RNA, 26 nt, Artificial Sequence
  Synthetic
cuuuccauggaagguucuaaaccauc

SEQ ID NO: 150 - RNA, 32 nt, Artificial Sequence; identical to SEQ ID NO: 63
  Synthetic

SEQ ID NO: 151 - RNA, 31 nt, Artificial Sequence; identical to SEQ ID NO: 135
  Synthetic

SEQ ID NO: 152 - RNA, 30 nt, Artificial Sequence; identical to SEQ ID NO: 136
  Synthetic

SEQ ID NO: 153 - RNA, 29 nt, Artificial Sequence
  Synthetic
ucucuuuccauggaagguucuaaaccauc

SEQ ID NO: 154 - RNA, 31 nt, Artificial Sequence; identical to SEQ ID NO: 138
  Synthetic

SEQ ID NO: 155 - RNA, 30 nt, Artificial Sequence; identical to SEQ ID NO: 139
  Synthetic

SEQ ID NO: 156 - RNA, 29 nt, Artificial Sequence
  Synthetic
cucuuuccauggaagguucuaaaccaucc

SEQ ID NO: 157 - RNA, 28 nt, Artificial Sequence
  Synthetic
cucuuuccauggaagguucuaaaccauc

SEQ ID NO: 158 - RNA, 30 nt, Artificial Sequence; identical to SEQ ID NO: 142
  Synthetic

SEQ ID NO: 159 - RNA, 29 nt, Artificial Sequence
  Synthetic
ucuuuccauggaagguucuaaaccauccu

SEQ ID NO: 160 - RNA, 28 nt, Artificial Sequence
  Synthetic
ucuuuccauggaagguucuaaaccaucc

SEQ ID NO: 161 - RNA, 27 nt, Artificial Sequence
  Synthetic
ucuuuccauggaagguucuaaaccauc

SEQ ID NO: 162 - RNA, 29 nt, Artificial Sequence
  Synthetic
cuuuccauggaagguucuaaaccauccug

SEQ ID NO: 163 - RNA, 28 nt, Artificial Sequence
  Synthetic
cuuuccauggaagguucuaaaccauccu

SEQ ID NO: 164 - RNA, 27 nt, Artificial Sequence
  Synthetic
cuuuccauggaagguucuaaaccaucc

SEQ ID NO: 165 - RNA, 26 nt, Artificial Sequence
  Synthetic
cuuuccauggaagguucuaaaccauc

SEQ ID NO: 166 - RNA, 32 nt, Artificial Sequence; identical to SEQ ID NO: 63
  Synthetic

SEQ ID NO: 167 - RNA, 31 nt, Artificial Sequence; identical to SEQ ID NO: 135
  Synthetic

SEQ ID NO: 168 - RNA, 30 nt, Artificial Sequence; identical to SEQ ID NO: 136
  Synthetic

SEQ ID NO: 169 - RNA, 29 nt, Artificial Sequence
  Synthetic
ucucuuuccauggaagguucuaaaccauc

SEQ ID NO: 170 - RNA, 31 nt, Artificial Sequence; identical to SEQ ID NO: 138
  Synthetic

SEQ ID NO: 171 - RNA, 30 nt, Artificial Sequence; identical to SEQ ID NO: 139
  Synthetic

SEQ ID NO: 172 - RNA, 29 nt, Artificial Sequence
  Synthetic
cucuuuccauggaagguucuaaaccaucc

SEQ ID NO: 173 - RNA, 28 nt, Artificial Sequence
  Synthetic
cucuuuccauggaagguucuaaaccauc

SEQ ID NO: 174 - RNA, 30 nt, Artificial Sequence; identical to SEQ ID NO: 142
  Synthetic

SEQ ID NO: 175 - RNA, 29 nt, Artificial Sequence
  Synthetic
ucuuuccauggaagguucuaaaccauccu

SEQ ID NO: 176 - RNA, 28 nt, Artificial Sequence
  Synthetic
ucuuuccauggaagguucuaaaccaucc

SEQ ID NO: 177 - RNA, 27 nt, Artificial Sequence
  Synthetic
ucuuuccauggaagguucuaaaccauc

SEQ ID NO: 178 - RNA, 29 nt, Artificial Sequence
  Synthetic
cuuuccauggaagguucuaaaccauccug

SEQ ID NO: 179 - RNA, 28 nt, Artificial Sequence
  Synthetic
cuuuccauggaagguucuaaaccauccu

SEQ ID NO: 180 - RNA, 27 nt, Artificial Sequence
  Synthetic
cuuuccauggaagguucuaaaccaucc

SEQ ID NO: 181 - RNA, 26 nt, Artificial Sequence
  Synthetic
cuuuccauggaagguucuaaaccauc

SEQ ID NO: 182 - RNA, 32 nt, Artificial Sequence; identical to SEQ ID NO: 63
  Synthetic

SEQ ID NO: 183 - RNA, 31 nt, Artificial Sequence; identical to SEQ ID NO: 135
  Synthetic

SEQ ID NO: 184 - RNA, 30 nt, Artificial Sequence; identical to SEQ ID NO: 136
  Synthetic

SEQ ID NO: 185 - RNA, 29 nt, Artificial Sequence
  Synthetic
ucucuuuccauggaagguucuaaaccauc

SEQ ID NO: 186 - RNA, 31 nt, Artificial Sequence; identical to SEQ ID NO: 138
  Synthetic

SEQ ID NO: 187 - RNA, 30 nt, Artificial Sequence; identical to SEQ ID NO: 139
  Synthetic

SEQ ID NO: 188 - RNA, 29 nt, Artificial Sequence
  Synthetic
cucuuuccauggaagguucuaaaccaucc

SEQ ID NO: 189 - RNA, 28 nt, Artificial Sequence
  Synthetic
cucuuuccauggaagguucuaaaccauc

SEQ ID NO: 190 - RNA, 30 nt, Artificial Sequence; identical to SEQ ID NO: 142
  Synthetic

SEQ ID NO: 191 - RNA, 29 nt, Artificial Sequence
  Synthetic
ucuuuccauggaagguucuaaaccauccu

SEQ ID NO: 192 - RNA, 28 nt, Artificial Sequence
  Synthetic
ucuuuccauggaagguucuaaaccaucc

SEQ ID NO: 193 - RNA, 27 nt, Artificial Sequence
  Synthetic
ucuuuccauggaagguucuaaaccauc

SEQ ID NO: 194 - RNA, 29 nt, Artificial Sequence
  Synthetic
cuuuccauggaagguucuaaaccauccug

SEQ ID NO: 195 - RNA, 28 nt, Artificial Sequence
  Synthetic
cuuuccauggaagguucuaaaccauccu

SEQ ID NO: 196 - RNA, 27 nt, Artificial Sequence
  Synthetic
cuuuccauggaagguucuaaaccaucc

SEQ ID NO: 197 - RNA, 26 nt, Artificial Sequence
  Synthetic
cuuuccauggaagguucuaaaccauc

SEQ ID NO: 198 - RNA, 31 nt, Artificial Sequence; identical to SEQ ID NO: 135
  Synthetic

SEQ ID NO: 199 - RNA, 30 nt, Artificial Sequence; identical to SEQ ID NO: 136
  Synthetic

SEQ ID NO: 200 - RNA, 29 nt, Artificial Sequence
  Synthetic
ucucuuuccauggaagguucuaaaccauc

SEQ ID NO: 201 - RNA, 31 nt, Artificial Sequence; identical to SEQ ID NO: 138
  Synthetic

SEQ ID NO: 202 - RNA, 30 nt, Artificial Sequence; identical to SEQ ID NO: 139
  Synthetic

SEQ ID NO: 203 - RNA, 29 nt, Artificial Sequence
  Synthetic
cucuuuccauggaagguucuaaaccaucc

SEQ ID NO: 204 - RNA, 28 nt, Artificial Sequence
  Synthetic
cucuuuccauggaagguucuaaaccauc

SEQ ID NO: 205 - RNA, 30 nt, Artificial Sequence; identical to SEQ ID NO: 142
  Synthetic

SEQ ID NO: 206 - RNA, 29 nt, Artificial Sequence
  Synthetic
ucuuuccauggaagguucuaaaccauccu

SEQ ID NO: 207 - RNA, 28 nt, Artificial Sequence
  Synthetic
ucuuuccauggaagguucuaaaccaucc

SEQ ID NO: 208 - RNA, 27 nt, Artificial Sequence
  Synthetic
ucuuuccauggaagguucuaaaccauc

SEQ ID NO: 209 - RNA, 29 nt, Artificial Sequence
  Synthetic
cuuuccauggaagguucuaaaccauccug

SEQ ID NO: 210 - RNA, 28 nt, Artificial Sequence
  Synthetic
cuuuccauggaagguucuaaaccauccu

SEQ ID NO: 211 - RNA, 27 nt, Artificial Sequence
  Synthetic
cuuuccauggaagguucuaaaccaucc

SEQ ID NO: 212 - RNA, 26 nt, Artificial Sequence
  Synthetic
cuuuccauggaagguucuaaaccauc

SEQ ID NO: 213 - RNA, 30 nt, Artificial Sequence
  Synthetic
ucucuuuccauugaaaguucuaaaccaucc

SEQ ID NO: 214 - RNA, 30 nt, Artificial Sequence
  Synthetic
ucucuuuccaaagaagguucuaaaccaucc

SEQ ID NO: 215 - RNA, 30 nt, Artificial Sequence
  Synthetic
ucucuuuccuuagaagguucuaaaccaucc

SEQ ID NO: 216 - RNA, 30 nt, Artificial Sequence
  Synthetic
ucucuuucgauagaagguucuaaaccaucc

SEQ ID NO: 217 - RNA, 30 nt, Artificial Sequence
  Synthetic
ucucuuugcauagaagguucuaaaccaucc

SEQ ID NO: 218 - RNA, 30 nt, Artificial Sequence
  Synthetic
ucucuauccauagaagguucuaaaccaucc

SEQ ID NO: 219 - RNA, 30 nt, Artificial Sequence
  Synthetic
ucucauuccauagaagguucuaaaccaucc

SEQ ID NO: 220 - RNA, 30 nt, Artificial Sequence
  Synthetic
ucuguuuccauagaagguucuaaaccaucc

SEQ ID NO: 221 - RNA, 30 nt, Artificial Sequence
  Synthetic
ucucuuuccauggaaucuucuaaaccaucc

SEQ ID NO: 222 - RNA, 30 nt, Artificial Sequence
  Synthetic
ucucuuuccauggaaugaucuaaaccaucc

SEQ ID NO: 223 - RNA, 30 nt, Artificial Sequence
  Synthetic
ucucuuuccauggaauguacuaaaccaucc

SEQ ID NO: 224 - RNA, 30 nt, Artificial Sequence
  Synthetic
ucucuuuccauggaauguuguaaaccaucc

SEQ ID NO: 225 - RNA, 30 nt, Artificial Sequence
  Synthetic
ucucuuuccauggaauguucaaaaccaucc

SEQ ID NO: 226 - RNA, 30 nt, Artificial Sequence
  Synthetic
ucucuuuccauggaauguucuuaaccaucc

SEQ ID NO: 227 - RNA, 30 nt, Artificial Sequence
  Synthetic
ucucuuuccauggaauguucuauaccaucc

SEQ ID NO: 228 - RNA, 30 nt, Artificial Sequence
  Synthetic
ucucuuuccauggaauguucuaauccaucc

SEQ ID NO: 229 - RNA, 30 nt, Artificial Sequence
  Synthetic
ucucuuuccaaagauuguucuaaaccaucc

SEQ ID NO: 230 - RNA, 30 nt, Artificial Sequence
  Synthetic
ucucuuuccuuaguauguucuaaaccaucc

SEQ ID NO: 231 - RNA, 30 nt, Artificial Sequence
  Synthetic
ucucuuucgauacaauguucuaaaccaucc

SEQ ID NO: 232 - RNA, 30 nt, Artificial Sequence
  Synthetic
ucucuuugcauugaauguucuaaaccaucc

SEQ ID NO: 233 - RNA, 30 nt, Artificial Sequence
  Synthetic
ucucuuaccaaagaauguucuaaaccaucc

SEQ ID NO: 234 - RNA, 30 nt, Artificial Sequence
  Synthetic
ucucuauccuuagaauguucuaaaccaucc

SEQ ID NO: 235 - RNA, 30 nt, Artificial Sequence
  Synthetic
ucucauucgauagaauguucuaaaccaucc

SEQ ID NO: 236 - RNA, 30 nt, Artificial Sequence
  Synthetic
ucuguuugcauagaauguucuaaaccaucc

SEQ ID NO: 237 - RNA, 30 nt, Artificial Sequence
  Synthetic
ucucuuuccauacaaucuucuaaaccaucc

SEQ ID NO: 238 - RNA, 30 nt, Artificial Sequence
  Synthetic
ucucuuuccauaguaugaucuaaaccaucc

SEQ ID NO: 239 - RNA, 30 nt, Artificial Sequence
  Synthetic
ucucuuuccauagauuguacuaaaccaucc

SEQ ID NO: 240 - RNA, 30 nt, Artificial Sequence
  Synthetic
ucucuuuccauagaaaguuguaaaccaucc

SEQ ID NO: 241 - RNA, 30 nt, Artificial Sequence
  Synthetic
ucucuuuccauagaaucuucaaaaccaucc

SEQ ID NO: 242 - RNA, 30 nt, Artificial Sequence
  Synthetic
ucucuuuccauagaaugaucuuaaccaucc

SEQ ID NO: 243 - RNA, 30 nt, Artificial Sequence
  Synthetic
ucucuuuccauagaauguacuauaccaucc

SEQ ID NO: 244 - RNA, 30 nt, Artificial Sequence
  Synthetic
ucucuuuccauagaauguuguaauccaucc

SEQ ID NO: 245 - RNA, 30 nt, Artificial Sequence
  Synthetic
ucucuuuccauagaagguucuaaaccaucc

SEQ ID NO: 246 - RNA, 30 nt, Artificial Sequence
  Synthetic
ucucuuuccauggaauguucuaaaccaucc

SEQ ID NO: 247 - RNA, 30 nt, Artificial Sequence
  Synthetic
ucucuuuccauagaauguucuaaaccaucc

SEQ ID NO: 248 - RNA, 32 nt, Artificial Sequence; identical to SEQ ID NO: 63
  Synthetic

SEQ ID NO: 249 - DNA, 32 bp, Artificial Sequence
  Synthetic
ucuctuuccauggaagguucuaaaccauccug

SEQ ID NO: 250 - DNA, 32 bp, Artificial Sequence
  Synthetic
ucucutuccauggaagguucuaaaccauccug

SEQ ID NO: 251 - DNA, 32 bp, Artificial Sequence
  Synthetic
ucucuutccauggaagguucuaaaccauccug

SEQ ID NO: 252 - RNA, 32 nt, Artificial Sequence; identical to SEQ ID NO: 63
  Synthetic

SEQ ID NO: 253 - RNA, 32 nt, Artificial Sequence; identical to SEQ ID NO: 63
  Synthetic

SEQ ID NO: 254 - RNA, 32 nt, Artificial Sequence; identical to SEQ ID NO: 63
  Synthetic

SEQ ID NO: 255 - DNA, 32 bp, Artificial Sequence
  Synthetic
ucucuuuccatggaagguucuaaaccauccug

SEQ ID NO: 256 - RNA, 32 nt, Artificial Sequence; identical to SEQ ID NO: 63
  Synthetic

SEQ ID NO: 257 - RNA, 32 nt, Artificial Sequence; identical to SEQ ID NO: 63
  Synthetic

SEQ ID NO: 258 - RNA, 32 nt, Artificial Sequence; identical to SEQ ID NO: 63
  Synthetic

SEQ ID NO: 259 - RNA, 32 nt, Artificial Sequence; identical to SEQ ID NO: 63
  Synthetic

SEQ ID NO: 260 - RNA, 32 nt, Artificial Sequence; identical to SEQ ID NO: 63
  Synthetic

SEQ ID NO: 261 - RNA, 32 nt, Artificial Sequence; identical to SEQ ID NO: 63
  Synthetic

SEQ ID NO: 262 - DNA, 32 bp, Artificial Sequence
  Synthetic
ucucuuuccauggaaggtucuaaaccauccug

SEQ ID NO: 263 - DNA, 32 bp, Artificial Sequence
  Synthetic
ucucuuuccauggaaggutcuaaaccauccug

SEQ ID NO: 264 - RNA, 32 nt, Artificial Sequence; identical to SEQ ID NO: 63
  Synthetic

SEQ ID NO: 265 - DNA, 32 bp, Artificial Sequence
  Synthetic
ucucuuuccauggaagguuctaaaccauccug

SEQ ID NO: 266 - RNA, 32 nt, Artificial Sequence; identical to SEQ ID NO: 63
  Synthetic

SEQ ID NO: 267 - RNA, 32 nt, Artificial Sequence; identical to SEQ ID NO: 63
  Synthetic

SEQ ID NO: 268 - RNA, 32 nt, Artificial Sequence; identical to SEQ ID NO: 63
  Synthetic

SEQ ID NO: 269 - RNA, 32 nt, Artificial Sequence; identical to SEQ ID NO: 63
  Synthetic

SEQ ID NO: 270 - RNA, 32 nt, Artificial Sequence; identical to SEQ ID NO: 63
  Synthetic

SEQ ID NO: 271 - RNA, 32 nt, Artificial Sequence; identical to SEQ ID NO: 63
  Synthetic

SEQ ID NO: 272 - RNA, 32 nt, Artificial Sequence; identical to SEQ ID NO: 63
  Synthetic

SEQ ID NO: 273 - RNA, 32 nt, Artificial Sequence; identical to SEQ ID NO: 63
  Synthetic

SEQ ID NO: 274 - RNA, 32 nt, Artificial Sequence; identical to SEQ ID NO: 63
  Synthetic

SEQ ID NO: 275 - DNA, 32 bp, Artificial Sequence
  Synthetic
ucucuuuccauggaagguucuaaaccatccug

SEQ ID NO: 276 - RNA, 32 nt, Artificial Sequence; identical to SEQ ID NO: 63
  Synthetic

SEQ ID NO: 277 - DNA, 32 bp, Artificial Sequence; identical to SEQ ID NO: 275
  Synthetic

SEQ ID NO: 278 - RNA, 32 nt, Artificial Sequence; identical to SEQ ID NO: 63
  Synthetic

SEQ ID NO: 279 - DNA, 32 bp, Artificial Sequence; identical to SEQ ID NO: 275
  Synthetic

SEQ ID NO: 280 - RNA, 32 nt, Artificial Sequence; identical to SEQ ID NO: 63
  Synthetic

SEQ ID NO: 281 - DNA, 32 bp, Artificial Sequence; identical to SEQ ID NO: 275
  Synthetic

SEQ ID NO: 282 - DNA, 32 bp, Artificial Sequence; identical to SEQ ID NO: 275
  Synthetic

SEQ ID NO: 283 - RNA, 32 nt, Artificial Sequence; identical to SEQ ID NO: 63
  Synthetic

SEQ ID NO: 284 - DNA, 32 bp, Artificial Sequence; identical to SEQ ID NO: 275
  Synthetic

SEQ ID NO: 285 - RNA, 32 nt, Artificial Sequence; identical to SEQ ID NO: 63
  Synthetic

SEQ ID NO: 286 - DNA, 32 bp, Artificial Sequence; identical to SEQ ID NO: 275
  Synthetic

SEQ ID NO: 287 - RNA, 32 nt, Artificial Sequence; identical to SEQ ID NO: 63
  Synthetic

SEQ ID NO: 288 - DNA, 32 bp, Artificial Sequence; identical to SEQ ID NO: 275
  Synthetic

SEQ ID NO: 289 - RNA, 32 nt, Artificial Sequence; identical to SEQ ID NO: 63
  Synthetic

SEQ ID NO: 290 - RNA, 32 nt, Artificial Sequence; identical to SEQ ID NO: 63
  Synthetic

SEQ ID NO: 291 - DNA, 32 bp, Artificial Sequence; identical to SEQ ID NO: 275
  Synthetic

SEQ ID NO: 292 - RNA, 32 nt, Artificial Sequence; identical to SEQ ID NO: 63
  Synthetic

SEQ ID NO: 293 - DNA, 32 bp, Artificial Sequence; identical to SEQ ID NO: 275
  Synthetic

SEQ ID NO: 294 - RNA, 32 nt, Artificial Sequence; identical to SEQ ID NO: 63
  Synthetic

SEQ ID NO: 295 - DNA, 32 bp, Artificial Sequence; identical to SEQ ID NO: 275
  Synthetic

SEQ ID NO: 296 - RNA, 32 nt, Artificial Sequence; identical to SEQ ID NO: 63
  Synthetic

SEQ ID NO: 297 - DNA, 32 bp, Artificial Sequence; identical to SEQ ID NO: 275
  Synthetic

SEQ ID NO: 298 - RNA, 32 nt, Artificial Sequence; identical to SEQ ID NO: 63
  Synthetic

SEQ ID NO: 299 - DNA, 32 bp, Artificial Sequence; identical to SEQ ID NO: 275
  Synthetic

SEQ ID NO: 300 - RNA, 32 nt, Artificial Sequence; identical to SEQ ID NO: 63
  Synthetic

SEQ ID NO: 301 - DNA, 32 bp, Artificial Sequence; identical to SEQ ID NO: 275
  Synthetic

SEQ ID NO: 302 - RNA, 32 nt, Artificial Sequence; identical to SEQ ID NO: 63
  Synthetic

SEQ ID NO: 303 - RNA, 31 nt, Artificial Sequence; identical to SEQ ID NO: 135
  Synthetic

SEQ ID NO: 304 - RNA, 30 nt, Artificial Sequence; identical to SEQ ID NO: 136
  Synthetic

SEQ ID NO: 305 - RNA, 29 nt, Artificial Sequence
  Synthetic
ucucuuuccauggaagguucuaaaccauc

SEQ ID NO: 306 - RNA, 31 nt, Artificial Sequence; identical to SEQ ID NO: 138
  Synthetic

SEQ ID NO: 307 - RNA, 30 nt, Artificial Sequence; identical to SEQ ID NO: 139
  Synthetic

SEQ ID NO: 308 - RNA, 29 nt, Artificial Sequence
  Synthetic
cucuuuccauggaagguucuaaaccaucc

SEQ ID NO: 309 - RNA, 28 nt, Artificial Sequence
  Synthetic
cucuuuccauggaagguucuaaaccauc

SEQ ID NO: 310 - RNA, 30 nt, Artificial Sequence; identical to SEQ ID NO: 142
  Synthetic

SEQ ID NO: 311 - RNA, 29 nt, Artificial Sequence
  Synthetic
ucuuuccauggaagguucuaaaccauccu

SEQ ID NO: 312 - RNA, 28 nt, Artificial Sequence
  Synthetic
ucuuuccauggaagguucuaaaccaucc

SEQ ID NO: 313 - RNA, 27 nt, Artificial Sequence
  Synthetic
ucuuuccauggaagguucuaaaccauc

SEQ ID NO: 314 - RNA, 29 nt, Artificial Sequence
  Synthetic
cuuuccauggaagguucuaaaccauccug

SEQ ID NO: 315 - RNA, 28 nt, Artificial Sequence
  Synthetic
cuuuccauggaagguucuaaaccauccu

SEQ ID NO: 316 - RNA, 27 nt, Artificial Sequence
  Synthetic
cuuuccauggaagguucuaaaccaucc

SEQ ID NO: 317 - RNA, 26 nt, Artificial Sequence
  Synthetic
cuuuccauggaagguucuaaaccauc

SEQ ID NO: 318 - RNA, 32 nt, Artificial Sequence; identical to SEQ ID NO: 63
  Synthetic

SEQ ID NO: 319 - RNA, 31 nt, Artificial Sequence; identical to SEQ ID NO: 135
  Synthetic

SEQ ID NO: 320 - RNA, 30 nt, Artificial Sequence; identical to SEQ ID NO: 136
  Synthetic

SEQ ID NO: 321 - RNA, 29 nt, Artificial Sequence
  Synthetic
ucucuuuccauggaagguucuaaaccauc

SEQ ID NO: 322 - RNA, 31 nt, Artificial Sequence; identical to SEQ ID NO: 138
  Synthetic

SEQ ID NO: 323 - RNA, 30 nt, Artificial Sequence; identical to SEQ ID NO: 139
  Synthetic

SEQ ID NO: 324 - RNA, 29 nt, Artificial Sequence
  Synthetic
cucuuuccauggaagguucuaaaccaucc

SEQ ID NO: 325 - RNA, 28 nt, Artificial Sequence
  Synthetic
cucuuuccauggaagguucuaaaccauc

SEQ ID NO: 326 - RNA, 30 nt, Artificial Sequence; identical to SEQ ID NO: 142
  Synthetic

SEQ ID NO: 327 - RNA, 29 nt, Artificial Sequence
  Synthetic
ucuuuccauggaagguucuaaaccauccu

SEQ ID NO: 328 - RNA, 28 nt, Artificial Sequence
  Synthetic
ucuuuccauggaagguucuaaaccaucc

SEQ ID NO: 329 - RNA, 27 nt, Artificial Sequence
  Synthetic
ucuuuccauggaagguucuaaaccauc

SEQ ID NO: 330 - RNA, 29 nt, Artificial Sequence
  Synthetic
cuuuccauggaagguucuaaaccauccug

SEQ ID NO: 331 - RNA, 28 nt, Artificial Sequence
  Synthetic
cuuuccauggaagguucuaaaccauccu

SEQ ID NO: 332 - RNA, 27 nt, Artificial Sequence
  Synthetic
cuuuccauggaagguucuaaaccaucc

SEQ ID NO: 333 - RNA, 26 nt, Artificial Sequence
  Synthetic
cuuuccauggaagguucuaaaccauc

SEQ ID NO: 334 - RNA, 32 nt, Artificial Sequence; identical to SEQ ID NO: 63
  Synthetic

SEQ ID NO: 335 - RNA, 31 nt, Artificial Sequence; identical to SEQ ID NO: 135
  Synthetic

SEQ ID NO: 336 - RNA, 30 nt, Artificial Sequence; identical to SEQ ID NO: 136
  Synthetic

SEQ ID NO: 337 - RNA, 29 nt, Artificial Sequence
  Synthetic
ucucuuuccauggaagguucuaaaccauc

SEQ ID NO: 338 - RNA, 31 nt, Artificial Sequence; identical to SEQ ID NO: 138
  Synthetic

SEQ ID NO: 339 - RNA, 30 nt, Artificial Sequence; identical to SEQ ID NO: 139
  Synthetic

SEQ ID NO: 340 - RNA, 29 nt, Artificial Sequence
  Synthetic
cucuuuccauggaagguucuaaaccaucc

SEQ ID NO: 341 - RNA, 28 nt, Artificial Sequence
  Synthetic
cucuuuccauggaagguucuaaaccauc

SEQ ID NO: 342 - RNA, 30 nt, Artificial Sequence; identical to SEQ ID NO: 142
  Synthetic

SEQ ID NO: 343 - RNA, 29 nt, Artificial Sequence
  Synthetic
ucuuuccauggaagguucuaaaccauccu

SEQ ID NO: 344 - RNA, 28 nt, Artificial Sequence
  Synthetic
ucuuuccauggaagguucuaaaccaucc

SEQ ID NO: 345 - RNA, 27 nt, Artificial Sequence
  Synthetic
ucuuuccauggaagguucuaaaccauc

SEQ ID NO: 346 - RNA, 29 nt, Artificial Sequence
  Synthetic
cuuuccauggaagguucuaaaccauccug

SEQ ID NO: 347 - RNA, 28 nt, Artificial Sequence
  Synthetic
cuuuccauggaagguucuaaaccauccu

SEQ ID NO: 348 - RNA, 27 nt, Artificial Sequence
  Synthetic
cuuuccauggaagguucuaaaccaucc

SEQ ID NO: 349 - RNA, 26 nt, Artificial Sequence
  Synthetic
cuuuccauggaagguucuaaaccauc

SEQ ID NO: 350 - RNA, 30 nt, Artificial Sequence; identical to SEQ ID NO: 139
  Synthetic

SEQ ID NO: 351 - RNA, 30 nt, Artificial Sequence; identical to SEQ ID NO: 139
  Synthetic

SEQ ID NO: 352 - RNA, 30 nt, Artificial Sequence; identical to SEQ ID NO: 139
  Synthetic

SEQ ID NO: 353 - RNA, 30 nt, Artificial Sequence; identical to SEQ ID NO: 139
  Synthetic

SEQ ID NO: 354 - RNA, 30 nt, Artificial Sequence; identical to SEQ ID NO: 139
  Synthetic

SEQ ID NO: 355 - RNA, 30 nt, Artificial Sequence; identical to SEQ ID NO: 139
  Synthetic

SEQ ID NO: 356 - RNA, 30 nt, Artificial Sequence; identical to SEQ ID NO: 139
  Synthetic

SEQ ID NO: 357 - RNA, 30 nt, Artificial Sequence; identical to SEQ ID NO: 139
  Synthetic

SEQ ID NO: 358 - RNA, 30 nt, Artificial Sequence; identical to SEQ ID NO: 139
  Synthetic

SEQ ID NO: 359 - RNA, 30 nt, Artificial Sequence; identical to SEQ ID NO: 139
  Synthetic

SEQ ID NO: 360 - RNA, 30 nt, Artificial Sequence; identical to SEQ ID NO: 139
  Synthetic

SEQ ID NO: 361 - RNA, 30 nt, Artificial Sequence; identical to SEQ ID NO: 139
  Synthetic

SEQ ID NO: 362 - RNA, 30 nt, Artificial Sequence; identical to SEQ ID NO: 139
  Synthetic

SEQ ID NO: 363 - RNA, 30 nt, Artificial Sequence; identical to SEQ ID NO: 139
  Synthetic

SEQ ID NO: 364 - RNA, 30 nt, Artificial Sequence; identical to SEQ ID NO: 139
  Synthetic

SEQ ID NO: 365 - RNA, 30 nt, Artificial Sequence; identical to SEQ ID NO: 139
  Synthetic

SEQ ID NO: 366 - RNA, 30 nt, Artificial Sequence; identical to SEQ ID NO: 139
  Synthetic

SEQ ID NO: 367 - RNA, 30 nt, Artificial Sequence; identical to SEQ ID NO: 139
  Synthetic

SEQ ID NO: 368 - RNA, 30 nt, Artificial Sequence; identical to SEQ ID NO: 139
  Synthetic

SEQ ID NO: 369 - RNA, 30 nt, Artificial Sequence; identical to SEQ ID NO: 139
  Synthetic

SEQ ID NO: 370 - RNA, 30 nt, Artificial Sequence; identical to SEQ ID NO: 139
  Synthetic

SEQ ID NO: 371 - RNA, 30 nt, Artificial Sequence; identical to SEQ ID NO: 139
  Synthetic

SEQ ID NO: 372 - RNA, 30 nt, Artificial Sequence; identical to SEQ ID NO: 139
  Synthetic

SEQ ID NO: 373 - RNA, 30 nt, Artificial Sequence; identical to SEQ ID NO: 139
  Synthetic

SEQ ID NO: 374 - RNA, 30 nt, Artificial Sequence; identical to SEQ ID NO: 139
  Synthetic

SEQ ID NO: 375 - RNA, 30 nt, Artificial Sequence; identical to SEQ ID NO: 139
  Synthetic

SEQ ID NO: 376 - RNA, 30 nt, Artificial Sequence; identical to SEQ ID NO: 139
  Synthetic

SEQ ID NO: 377 - RNA, 30 nt, Artificial Sequence; identical to SEQ ID NO: 139
  Synthetic

SEQ ID NO: 378 - RNA, 30 nt, Artificial Sequence; identical to SEQ ID NO: 139
  Synthetic

SEQ ID NO: 379 - RNA, 30 nt, Artificial Sequence; identical to SEQ ID NO: 139
  Synthetic

SEQ ID NO: 380 - RNA, 30 nt, Artificial Sequence; identical to SEQ ID NO: 139
  Synthetic

SEQ ID NO: 381 - RNA, 30 nt, Artificial Sequence; identical to SEQ ID NO: 139
  Synthetic

SEQ ID NO: 382 - RNA, 30 nt, Artificial Sequence; identical to SEQ ID NO: 139
  Synthetic

SEQ ID NO: 383 - RNA, 30 nt, Artificial Sequence; identical to SEQ ID NO: 139
  Synthetic

SEQ ID NO: 384 - RNA, 30 nt, Artificial Sequence; identical to SEQ ID NO: 139
  Synthetic

SEQ ID NO: 385 - RNA, 30 nt, Artificial Sequence; identical to SEQ ID NO: 139
  Synthetic

SEQ ID NO: 386 - RNA, 30 nt, Artificial Sequence; identical to SEQ ID NO: 139
  Synthetic

SEQ ID NO: 387 - RNA, 30 nt, Artificial Sequence; identical to SEQ ID NO: 139
  Synthetic

SEQ ID NO: 388 - RNA, 30 nt, Artificial Sequence; identical to SEQ ID NO: 139
  Synthetic

SEQ ID NO: 389 - RNA, 30 nt, Artificial Sequence; identical to SEQ ID NO: 139
  Synthetic

SEQ ID NO: 390 - RNA, 30 nt, Artificial Sequence; identical to SEQ ID NO: 139
  Synthetic

SEQ ID NO: 391 - RNA, 30 nt, Artificial Sequence; identical to SEQ ID NO: 139
  Synthetic

SEQ ID NO: 392 - RNA, 30 nt, Artificial Sequence; identical to SEQ ID NO: 139
  Synthetic

SEQ ID NO: 393 - RNA, 30 nt, Artificial Sequence; identical to SEQ ID NO: 139
  Synthetic

SEQ ID NO: 394 - RNA, 30 nt, Artificial Sequence; identical to SEQ ID NO: 139
  Synthetic

SEQ ID NO: 395 - RNA, 30 nt, Artificial Sequence; identical to SEQ ID NO: 139
  Synthetic

SEQ ID NO: 396 - RNA, 30 nt, Artificial Sequence; identical to SEQ ID NO: 139
  Synthetic

SEQ ID NO: 397 - RNA, 30 nt, Artificial Sequence; identical to SEQ ID NO: 139
  Synthetic

SEQ ID NO: 398 - RNA, 32 nt, Artificial Sequence
  Synthetic
ucucuuuccauggaagguucuaaacuauccug

SEQ ID NO: 399 - RNA, 32 nt, Artificial Sequence; identical to SEQ ID NO: 398
  Synthetic

SEQ ID NO: 400 - RNA, 30 nt, Artificial Sequence; identical to SEQ ID NO: 139
  Synthetic

SEQ ID NO: 401 - RNA, 30 nt, Artificial Sequence; identical to SEQ ID NO: 139
  Synthetic

SEQ ID NO: 402 - RNA, 30 nt, Artificial Sequence; identical to SEQ ID NO: 139
  Synthetic

SEQ ID NO: 403 - RNA, 30 nt, Artificial Sequence; identical to SEQ ID NO: 139
  Synthetic

SEQ ID NO: 404 - RNA, 30 nt, Artificial Sequence; identical to SEQ ID NO: 139
  Synthetic

SEQ ID NO: 405 - RNA, 30 nt, Artificial Sequence; identical to SEQ ID NO: 139
  Synthetic

SEQ ID NO: 406 - RNA, 30 nt, Artificial Sequence; identical to SEQ ID NO: 139
  Synthetic

SEQ ID NO: 407 - RNA, 30 nt, Artificial Sequence; identical to SEQ ID NO: 139
  Synthetic

SEQ ID NO: 408 - RNA, 30 nt, Artificial Sequence; identical to SEQ ID NO: 139
  Synthetic

SEQ ID NO: 409 - RNA, 30 nt, Artificial Sequence; identical to SEQ ID NO: 139
  Synthetic

SEQ ID NO: 410 - RNA, 30 nt, Artificial Sequence; identical to SEQ ID NO: 139
  Synthetic

SEQ ID NO: 411 - RNA, 30 nt, Artificial Sequence; identical to SEQ ID NO: 139
  Synthetic

SEQ ID NO: 412 - RNA, 30 nt, Artificial Sequence; identical to SEQ ID NO: 139
  Synthetic

SEQ ID NO: 413 - RNA, 30 nt, Artificial Sequence; identical to SEQ ID NO: 139
  Synthetic

SEQ ID NO: 414 - RNA, 30 nt, Artificial Sequence; identical to SEQ ID NO: 139
  Synthetic

SEQ ID NO: 415 - RNA, 30 nt, Artificial Sequence; identical to SEQ ID NO: 139
  Synthetic

SEQ ID NO: 416 - RNA, 30 nt, Artificial Sequence; identical to SEQ ID NO: 139
  Synthetic

SEQ ID NO: 417 - RNA, 30 nt, Artificial Sequence; identical to SEQ ID NO: 139
  Synthetic

SEQ ID NO: 418 - RNA, 30 nt, Artificial Sequence; identical to SEQ ID NO: 139
  Synthetic

SEQ ID NO: 419 - RNA, 30 nt, Artificial Sequence; identical to SEQ ID NO: 139
  Synthetic

SEQ ID NO: 420 - RNA, 30 nt, Artificial Sequence; identical to SEQ ID NO: 139
  Synthetic

SEQ ID NO: 421 - RNA, 30 nt, Artificial Sequence; identical to SEQ ID NO: 139
  Synthetic

SEQ ID NO: 422 - RNA, 30 nt, Artificial Sequence; identical to SEQ ID NO: 139
  Synthetic

SEQ ID NO: 423 - RNA, 30 nt, Artificial Sequence; identical to SEQ ID NO: 139
  Synthetic

SEQ ID NO: 424 - RNA, 30 nt, Artificial Sequence; identical to SEQ ID NO: 139
  Synthetic

SEQ ID NO: 425 - RNA, 30 nt, Artificial Sequence; identical to SEQ ID NO: 139
  Synthetic

SEQ ID NO: 426 - RNA, 30 nt, Artificial Sequence; identical to SEQ ID NO: 139
  Synthetic

SEQ ID NO: 427 - RNA, 30 nt, Artificial Sequence; identical to SEQ ID NO: 139
  Synthetic

SEQ ID NO: 428 - RNA, 30 nt, Artificial Sequence; identical to SEQ ID NO: 139
  Synthetic

SEQ ID NO: 429 - RNA, 30 nt, Artificial Sequence; identical to SEQ ID NO: 139
  Synthetic

SEQ ID NO: 430 - RNA, 30 nt, Artificial Sequence
  Synthetic
cucuuuccauggaagguucuaaacuauccu

SEQ ID NO: 431 - RNA, 32 nt, Artificial Sequence; identical to SEQ ID NO: 398
  Synthetic

SEQ ID NO: 432 - RNA, 32 nt, Artificial Sequence; identical to SEQ ID NO: 398
  Synthetic

SEQ ID NO: 433 - RNA, 32 nt, Artificial Sequence; identical to SEQ ID NO: 398
  Synthetic

SEQ ID NO: 434 - RNA, 32 nt, Artificial Sequence; identical to SEQ ID NO: 398
  Synthetic

SEQ ID NO: 435 - RNA, 32 nt, Artificial Sequence
  Synthetic
ucucuuuccauggaagguucuaaacaauccug

SEQ ID NO: 436 - RNA, 30 nt, Artificial Sequence; identical to SEQ ID NO: 430
  Synthetic

SEQ ID NO: 437 - RNA, 30 nt, Artificial Sequence; identical to SEQ ID NO: 430
  Synthetic

SEQ ID NO: 438 - RNA, 30 nt, Artificial Sequence; identical to SEQ ID NO: 430
  Synthetic

SEQ ID NO: 439 - RNA, 30 nt, Artificial Sequence; identical to SEQ ID NO: 430
  Synthetic

SEQ ID NO: 440 - RNA, 30 nt, Artificial Sequence; identical to SEQ ID NO: 430
  Synthetic

SEQ ID NO: 441 - RNA, 30 nt, Artificial Sequence
  Synthetic
cucuuuccauggaagguucuaaacaauccu

SEQ ID NO: 442 - RNA, 32 nt, Artificial Sequence; identical to SEQ ID NO: 63
  Synthetic

SEQ ID NO: 443 - RNA, 32 nt, Artificial Sequence; identical to SEQ ID NO: 63
  Synthetic

SEQ ID NO: 444 - RNA, 30 nt, Artificial Sequence; identical to SEQ ID NO: 139
  Synthetic

SEQ ID NO: 445 - RNA, 30 nt, Artificial Sequence; identical to SEQ ID NO: 139
  Synthetic

SEQ ID NO: 446 - RNA, 32 nt, Artificial Sequence; identical to SEQ ID NO: 63
  Synthetic

SEQ ID NO: 447 - RNA, 30 nt, Artificial Sequence; identical to SEQ ID NO: 139
  Synthetic

SEQ ID NO: 448 - RNA, 30 nt, Artificial Sequence; identical to SEQ ID NO: 139
  Synthetic

SEQ ID NO: 449 - RNA, 30 nt, Artificial Sequence; identical to SEQ ID NO: 139
  Synthetic

SEQ ID NO: 450 - RNA, 30 nt, Artificial Sequence; identical to SEQ ID NO: 139
  Synthetic

SEQ ID NO: 451 - RNA, 30 nt, Artificial Sequence; identical to SEQ ID NO: 139
  Synthetic

SEQ ID NO: 452 - RNA, 30 nt, Artificial Sequence; identical to SEQ ID NO: 139
  Synthetic

SEQ ID NO: 453 - RNA, 30 nt, Artificial Sequence; identical to SEQ ID NO: 139
  Synthetic

SEQ ID NO: 454 - RNA, 30 nt, Artificial Sequence; identical to SEQ ID NO: 139
  Synthetic

SEQ ID NO: 455 - RNA, 30 nt, Artificial Sequence; identical to SEQ ID NO: 139
  Synthetic

SEQ ID NO: 456 - RNA, 30 nt, Artificial Sequence; identical to SEQ ID NO: 139
  Synthetic

SEQ ID NO: 457 - RNA, 30 nt, Artificial Sequence; identical to SEQ ID NO: 139
  Synthetic

SEQ ID NO: 458 - RNA, 30 nt, Artificial Sequence; identical to SEQ ID NO: 139
  Synthetic

SEQ ID NO: 459 - RNA, 30 nt, Artificial Sequence; identical to SEQ ID NO: 139
  Synthetic

SEQ ID NO: 460 - RNA, 30 nt, Artificial Sequence; identical to SEQ ID NO: 139
  Synthetic

SEQ ID NO: 461 - RNA, 30 nt, Artificial Sequence; identical to SEQ ID NO: 139
  Synthetic

SEQ ID NO: 462 - RNA, 30 nt, Artificial Sequence; identical to SEQ ID NO: 139
  Synthetic

SEQ ID NO: 463 - RNA, 30 nt, Artificial Sequence; identical to SEQ ID NO: 139
  Synthetic

SEQ ID NO: 464 - RNA, 30 nt, Artificial Sequence; identical to SEQ ID NO: 139
  Synthetic

SEQ ID NO: 465 - RNA, 30 nt, Artificial Sequence; identical to SEQ ID NO: 139
  Synthetic

SEQ ID NO: 466 - RNA, 30 nt, Artificial Sequence; identical to SEQ ID NO: 139
  Synthetic

SEQ ID NO: 467 - RNA, 30 nt, Artificial Sequence; identical to SEQ ID NO: 139
  Synthetic

SEQ ID NO: 468 - RNA, 30 nt, Artificial Sequence; identical to SEQ ID NO: 139
  Synthetic

SEQ ID NO: 469 - RNA, 30 nt, Artificial Sequence; identical to SEQ ID NO: 139
  Synthetic

SEQ ID NO: 470 - RNA, 30 nt, Artificial Sequence; identical to SEQ ID NO: 139
  Synthetic

SEQ ID NO: 471 - RNA, 30 nt, Artificial Sequence; identical to SEQ ID NO: 139
  Synthetic

SEQ ID NO: 472 - RNA, 30 nt, Artificial Sequence; identical to SEQ ID NO: 139
  Synthetic

SEQ ID NO: 473 - RNA, 30 nt, Artificial Sequence; identical to SEQ ID NO: 139
  Synthetic

SEQ ID NO: 474 - RNA, 30 nt, Artificial Sequence; identical to SEQ ID NO: 139
  Synthetic

SEQ ID NO: 475 - RNA, 30 nt, Artificial Sequence; identical to SEQ ID NO: 139
  Synthetic

SEQ ID NO: 476 - RNA, 30 nt, Artificial Sequence; identical to SEQ ID NO: 139
  Synthetic

SEQ ID NO: 477 - RNA, 30 nt, Artificial Sequence; identical to SEQ ID NO: 139
  Synthetic

SEQ ID NO: 478 - RNA, 30 nt, Artificial Sequence; identical to SEQ ID NO: 139
  Synthetic

SEQ ID NO: 479 - RNA, 30 nt, Artificial Sequence; identical to SEQ ID NO: 139
  Synthetic

SEQ ID NO: 480 - RNA, 30 nt, Artificial Sequence; identical to SEQ ID NO: 139
  Synthetic

SEQ ID NO: 481 - RNA, 30 nt, Artificial Sequence; identical to SEQ ID NO: 139
  Synthetic

SEQ ID NO: 482 - RNA, 30 nt, Artificial Sequence; identical to SEQ ID NO: 139
  Synthetic

SEQ ID NO: 483 - RNA, 30 nt, Artificial Sequence; identical to SEQ ID NO: 139
  Synthetic

SEQ ID NO: 484 - RNA, 30 nt, Artificial Sequence; identical to SEQ ID NO: 139
  Synthetic

SEQ ID NO: 485 - RNA, 30 nt, Artificial Sequence; identical to SEQ ID NO: 139
  Synthetic

SEQ ID NO: 486 - RNA, 30 nt, Artificial Sequence; identical to SEQ ID NO: 139
  Synthetic

SEQ ID NO: 487 - RNA, 30 nt, Artificial Sequence; identical to SEQ ID NO: 139
  Synthetic

SEQ ID NO: 488 - RNA, 30 nt, Artificial Sequence; identical to SEQ ID NO: 139
  Synthetic

SEQ ID NO: 489 - RNA, 30 nt, Artificial Sequence; identical to SEQ ID NO: 139
  Synthetic

SEQ ID NO: 490 - RNA, 30 nt, Artificial Sequence; identical to SEQ ID NO: 139
  Synthetic

SEQ ID NO: 491 - RNA, 30 nt, Artificial Sequence; identical to SEQ ID NO: 139
  Synthetic

SEQ ID NO: 492 - RNA, 30 nt, Artificial Sequence; identical to SEQ ID NO: 139
  Synthetic

SEQ ID NO: 493 - RNA, 30 nt, Artificial Sequence; identical to SEQ ID NO: 139
  Synthetic

SEQ ID NO: 494 - RNA, 30 nt, Artificial Sequence; identical to SEQ ID NO: 139
  Synthetic

SEQ ID NO: 495 - RNA, 30 nt, Artificial Sequence; identical to SEQ ID NO: 139
  Synthetic

SEQ ID NO: 496 - RNA, 30 nt, Artificial Sequence; identical to SEQ ID NO: 139
  Synthetic

SEQ ID NO: 497 - RNA, 30 nt, Artificial Sequence; identical to SEQ ID NO: 139
  Synthetic

SEQ ID NO: 498 - RNA, 30 nt, Artificial Sequence; identical to SEQ ID NO: 139
  Synthetic

SEQ ID NO: 499 - RNA, 30 nt, Artificial Sequence; identical to SEQ ID NO: 139
  Synthetic

SEQ ID NO: 500 - RNA, 30 nt, Artificial Sequence; identical to SEQ ID NO: 139
  Synthetic

SEQ ID NO: 501 - RNA, 30 nt, Artificial Sequence; identical to SEQ ID NO: 139
  Synthetic

SEQ ID NO: 502 - RNA, 30 nt, Artificial Sequence; identical to SEQ ID NO: 139
  Synthetic

SEQ ID NO: 503 - RNA, 30 nt, Artificial Sequence; identical to SEQ ID NO: 139
  Synthetic

SEQ ID NO: 504 - DNA, 30 bp, Artificial Sequence
  Synthetic
ctcuuuccauggaagguucuaaaccauccu

SEQ ID NO: 505 - DNA, 30 bp, Artificial Sequence
  Synthetic
cuctuuccauggaagguucuaaaccauccu

SEQ ID NO: 506 - DNA, 30 bp, Artificial Sequence
  Synthetic
cucutuccauggaagguucuaaaccauccu

SEQ ID NO: 507 - DNA, 30 bp, Artificial Sequence
  Synthetic
cucuutccauggaagguucuaaaccauccu

SEQ ID NO: 508 - DNA, 30 bp, Artificial Sequence
  Synthetic
cucuuuccatggaagguucuaaaccauccu

SEQ ID NO: 509 - DNA, 30 bp, Artificial Sequence
  Synthetic
cucuuuccauggaaggtucuaaaccauccu

SEQ ID NO: 510 - DNA, 30 bp, Artificial Sequence
  Synthetic
cucuuuccauggaaggutcuaaaccauccu

SEQ ID NO: 511 - DNA, 30 bp, Artificial Sequence
  Synthetic
cucuuuccauggaagguuctaaaccauccu

SEQ ID NO: 512 - DNA, 30 bp, Artificial Sequence
  Synthetic
cucuuuccauggaagguucuaaaccatccu

SEQ ID NO: 513 - DNA, 30 bp, Artificial Sequence
  Synthetic
cucuuuccauggaagguucuaaaccaucct

SEQ ID NO: 514 - RNA, 30 nt, Artificial Sequence; identical to SEQ ID NO: 139
  Synthetic

SEQ ID NO: 515 - RNA, 30 nt, Artificial Sequence; identical to SEQ ID NO: 139
  Synthetic

SEQ ID NO: 516 - RNA, 30 nt, Artificial Sequence; identical to SEQ ID NO: 139
  Synthetic

SEQ ID NO: 517 - RNA, 30 nt, Artificial Sequence; identical to SEQ ID NO: 139
  Synthetic

SEQ ID NO: 518 - RNA, 30 nt, Artificial Sequence; identical to SEQ ID NO: 139
  Synthetic

SEQ ID NO: 519 - RNA, 30 nt, Artificial Sequence; identical to SEQ ID NO: 139
  Synthetic

SEQ ID NO: 520 - RNA, 30 nt, Artificial Sequence; identical to SEQ ID NO: 139
  Synthetic

SEQ ID NO: 521 - RNA, 30 nt, Artificial Sequence; identical to SEQ ID NO: 139
  Synthetic

SEQ ID NO: 522 - RNA, 30 nt, Artificial Sequence; identical to SEQ ID NO: 139
  Synthetic

SEQ ID NO: 523 - DNA, 30 bp, Artificial Sequence
  Synthetic
ctctuuccauggaagguucuaaaccauccu

SEQ ID NO: 524 - DNA, 30 bp, Artificial Sequence; identical to SEQ ID NO: 523
  Synthetic

SEQ ID NO: 525 - RNA, 30 nt, Artificial Sequence; identical to SEQ ID NO: 139
  Synthetic

SEQ ID NO: 526 - RNA, 30 nt, Artificial Sequence; identical to SEQ ID NO: 139
  Synthetic

SEQ ID NO: 527 - RNA, 30 nt, Artificial Sequence; identical to SEQ ID NO: 139
  Synthetic

SEQ ID NO: 528 - RNA, 30 nt, Artificial Sequence; identical to SEQ ID NO: 139
  Synthetic

SEQ ID NO: 529 - RNA, 30 nt, Artificial Sequence; identical to SEQ ID NO: 139
  Synthetic

SEQ ID NO: 530 - RNA, 30 nt, Artificial Sequence; identical to SEQ ID NO: 139
  Synthetic

SEQ ID NO: 531 - RNA, 30 nt, Artificial Sequence; identical to SEQ ID NO: 139
  Synthetic

SEQ ID NO: 532 - RNA, 30 nt, Artificial Sequence; identical to SEQ ID NO: 139
  Synthetic

SEQ ID NO: 533 - RNA, 30 nt, Artificial Sequence; identical to SEQ ID NO: 139
  Synthetic

SEQ ID NO: 534 - RNA, 30 nt, Artificial Sequence; identical to SEQ ID NO: 139
  Synthetic

SEQ ID NO: 535 - RNA, 30 nt, Artificial Sequence; identical to SEQ ID NO: 139
  Synthetic

SEQ ID NO: 536 - RNA, 30 nt, Artificial Sequence; identical to SEQ ID NO: 139
  Synthetic

SEQ ID NO: 537 - RNA, 30 nt, Artificial Sequence; identical to SEQ ID NO: 139
  Synthetic

SEQ ID NO: 538 - RNA, 30 nt, Artificial Sequence; identical to SEQ ID NO: 139
  Synthetic

SEQ ID NO: 539 - RNA, 30 nt, Artificial Sequence; identical to SEQ ID NO: 139
  Synthetic

SEQ ID NO: 540 - RNA, 30 nt, Artificial Sequence; identical to SEQ ID NO: 139
  Synthetic

SEQ ID NO: 541 - RNA, 30 nt, Artificial Sequence; identical to SEQ ID NO: 139
  Synthetic

SEQ ID NO: 542 - RNA, 30 nt, Artificial Sequence; identical to SEQ ID NO: 139
  Synthetic

SEQ ID NO: 543 - RNA, 30 nt, Artificial Sequence; identical to SEQ ID NO: 139
  Synthetic

SEQ ID NO: 544 - RNA, 30 nt, Artificial Sequence; identical to SEQ ID NO: 139
  Synthetic

SEQ ID NO: 545 - RNA, 30 nt, Artificial Sequence; identical to SEQ ID NO: 139
  Synthetic

SEQ ID NO: 546 - RNA, 30 nt, Artificial Sequence; identical to SEQ ID NO: 139
  Synthetic

SEQ ID NO: 547 - RNA, 30 nt, Artificial Sequence; identical to SEQ ID NO: 139
  Synthetic

SEQ ID NO: 548 - RNA, 30 nt, Artificial Sequence; identical to SEQ ID NO: 139
  Synthetic

SEQ ID NO: 549 - RNA, 30 nt, Artificial Sequence; identical to SEQ ID NO: 139
  Synthetic

SEQ ID NO: 550 - RNA, 30 nt, Artificial Sequence; identical to SEQ ID NO: 139
  Synthetic

SEQ ID NO: 551 - RNA, 30 nt, Artificial Sequence; identical to SEQ ID NO: 139
  Synthetic

SEQ ID NO: 552 - RNA, 30 nt, Artificial Sequence; identical to SEQ ID NO: 139
  Synthetic

SEQ ID NO: 553 - RNA, 30 nt, Artificial Sequence; identical to SEQ ID NO: 139
  Synthetic

SEQ ID NO: 554 - RNA, 30 nt, Artificial Sequence; identical to SEQ ID NO: 139
  Synthetic

SEQ ID NO: 555 - RNA, 30 nt, Artificial Sequence; identical to SEQ ID NO: 139
  Synthetic

SEQ ID NO: 556 - RNA, 30 nt, Artificial Sequence; identical to SEQ ID NO: 139
  Synthetic

SEQ ID NO: 557 - RNA, 30 nt, Artificial Sequence; identical to SEQ ID NO: 139
  Synthetic

SEQ ID NO: 558 - RNA, 30 nt, Artificial Sequence; identical to SEQ ID NO: 139
  Synthetic

SEQ ID NO: 559 - RNA, 30 nt, Artificial Sequence; identical to SEQ ID NO: 139
  Synthetic

SEQ ID NO: 560 - RNA, 30 nt, Artificial Sequence; identical to SEQ ID NO: 139
  Synthetic

SEQ ID NO: 561 - RNA, 30 nt, Artificial Sequence; identical to SEQ ID NO: 139
  Synthetic

SEQ ID NO: 562 - RNA, 30 nt, Artificial Sequence; identical to SEQ ID NO: 139
  Synthetic

SEQ ID NO: 563 - RNA, 30 nt, Artificial Sequence; identical to SEQ ID NO: 139
  Synthetic

SEQ ID NO: 564 - RNA, 30 nt, Artificial Sequence; identical to SEQ ID NO: 139
  Synthetic

SEQ ID NO: 565 - RNA, 30 nt, Artificial Sequence; identical to SEQ ID NO: 139
  Synthetic

SEQ ID NO: 566 - RNA, 30 nt, Artificial Sequence; identical to SEQ ID NO: 139
  Synthetic

SEQ ID NO: 567 - RNA, 30 nt, Artificial Sequence; identical to SEQ ID NO: 139
  Synthetic

SEQ ID NO: 568 - RNA, 30 nt, Artificial Sequence; identical to SEQ ID NO: 139
  Synthetic

SEQ ID NO: 569 - RNA, 30 nt, Artificial Sequence; identical to SEQ ID NO: 139
  Synthetic

SEQ ID NO: 570 - RNA, 30 nt, Artificial Sequence; identical to SEQ ID NO: 139
  Synthetic

SEQ ID NO: 571 - RNA, 30 nt, Artificial Sequence; identical to SEQ ID NO: 139
  Synthetic

SEQ ID NO: 572 - RNA, 30 nt, Artificial Sequence; identical to SEQ ID NO: 139
  Synthetic

SEQ ID NO: 573 - RNA, 30 nt, Artificial Sequence; identical to SEQ ID NO: 139
  Synthetic

SEQ ID NO: 574 - RNA, 30 nt, Artificial Sequence; identical to SEQ ID NO: 139
  Synthetic

SEQ ID NO: 575 - RNA, 30 nt, Artificial Sequence; identical to SEQ ID NO: 139
  Synthetic

SEQ ID NO: 576 - RNA, 30 nt, Artificial Sequence; identical to SEQ ID NO: 139
  Synthetic

SEQ ID NO: 577 - RNA, 30 nt, Artificial Sequence; identical to SEQ ID NO: 139
  Synthetic

SEQ ID NO: 578 - RNA, 30 nt, Artificial Sequence; identical to SEQ ID NO: 139
  Synthetic

SEQ ID NO: 579 - RNA, 30 nt, Artificial Sequence; identical to SEQ ID NO: 139
  Synthetic

SEQ ID NO: 580 - RNA, 30 nt, Artificial Sequence; identical to SEQ ID NO: 139
  Synthetic

SEQ ID NO: 581 - RNA, 30 nt, Artificial Sequence; identical to SEQ ID NO: 139
  Synthetic

SEQ ID NO: 582 - RNA, 30 nt, Artificial Sequence; identical to SEQ ID NO: 139
  Synthetic

SEQ ID NO: 583 - RNA, 30 nt, Artificial Sequence; identical to SEQ ID NO: 139
  Synthetic

SEQ ID NO: 584 - RNA, 30 nt, Artificial Sequence; identical to SEQ ID NO: 139
  Synthetic

SEQ ID NO: 585 - RNA, 30 nt, Artificial Sequence; identical to SEQ ID NO: 139
  Synthetic

SEQ ID NO: 586 - RNA, 30 nt, Artificial Sequence; identical to SEQ ID NO: 139
  Synthetic

SEQ ID NO: 587 - RNA, 30 nt, Artificial Sequence; identical to SEQ ID NO: 139
  Synthetic

SEQ ID NO: 588 - RNA, 30 nt, Artificial Sequence; identical to SEQ ID NO: 139
  Synthetic

SEQ ID NO: 589 - RNA, 30 nt, Artificial Sequence; identical to SEQ ID NO: 139
  Synthetic

SEQ ID NO: 590 - RNA, 30 nt, Artificial Sequence; identical to SEQ ID NO: 139
  Synthetic

SEQ ID NO: 591 - RNA, 30 nt, Artificial Sequence; identical to SEQ ID NO: 139
  Synthetic

SEQ ID NO: 592 - RNA, 30 nt, Artificial Sequence; identical to SEQ ID NO: 139
  Synthetic

SEQ ID NO: 593 - RNA, 30 nt, Artificial Sequence; identical to SEQ ID NO: 139
  Synthetic

SEQ ID NO: 594 - RNA, 30 nt, Artificial Sequence; identical to SEQ ID NO: 139
  Synthetic

SEQ ID NO: 595 - RNA, 30 nt, Artificial Sequence; identical to SEQ ID NO: 139
  Synthetic

SEQ ID NO: 596 - RNA, 30 nt, Artificial Sequence; identical to SEQ ID NO: 139
  Synthetic

SEQ ID NO: 597 - RNA, 30 nt, Artificial Sequence; identical to SEQ ID NO: 139
  Synthetic

SEQ ID NO: 598 - RNA, 30 nt, Artificial Sequence; identical to SEQ ID NO: 139
  Synthetic

SEQ ID NO: 599 - RNA, 30 nt, Artificial Sequence; identical to SEQ ID NO: 139
  Synthetic

SEQ ID NO: 600 - RNA, 30 nt, Artificial Sequence; identical to SEQ ID NO: 139
  Synthetic

SEQ ID NO: 601 - RNA, 30 nt, Artificial Sequence; identical to SEQ ID NO: 139
  Synthetic

SEQ ID NO: 602 - RNA, 30 nt, Artificial Sequence; identical to SEQ ID NO: 139
  Synthetic

SEQ ID NO: 603 - RNA, 30 nt, Artificial Sequence; identical to SEQ ID NO: 139
  Synthetic

SEQ ID NO: 604 - RNA, 30 nt, Artificial Sequence; identical to SEQ ID NO: 139
  Synthetic

SEQ ID NO: 605 - RNA, 30 nt, Artificial Sequence; identical to SEQ ID NO: 139
  Synthetic

SEQ ID NO: 606 - RNA, 30 nt, Artificial Sequence; identical to SEQ ID NO: 139
  Synthetic

SEQ ID NO: 607 - RNA, 30 nt, Artificial Sequence; identical to SEQ ID NO: 139
  Synthetic

SEQ ID NO: 608 - RNA, 30 nt, Artificial Sequence; identical to SEQ ID NO: 139
  Synthetic

SEQ ID NO: 609 - RNA, 30 nt, Artificial Sequence; identical to SEQ ID NO: 139
  Synthetic

SEQ ID NO: 610 - RNA, 30 nt, Artificial Sequence; identical to SEQ ID NO: 139
  Synthetic

SEQ ID NO: 611 - RNA, 30 nt, Artificial Sequence; identical to SEQ ID NO: 139
  Synthetic

SEQ ID NO: 612 - RNA, 30 nt, Artificial Sequence; identical to SEQ ID NO: 139
  Synthetic

SEQ ID NO: 613 - RNA, 30 nt, Artificial Sequence; identical to SEQ ID NO: 139
  Synthetic

SEQ ID NO: 614 - RNA, 30 nt, Artificial Sequence; identical to SEQ ID NO: 139
  Synthetic

SEQ ID NO: 615 - RNA, 32 nt, Artificial Sequence
  Synthetic
gccccagcagcaucacucccuuucucgucgau

SEQ ID NO: 616 - DNA, 30 bp, Artificial Sequence
  Synthetic
ccccagcagcaucacucccuuuctcgucga

SEQ ID NO: 617 - RNA, 32 nt, Artificial Sequence
  Synthetic
  misc_feature (27)..(27): n is the nucleobase hypoxanthine
gccccagcagcaucacucccuuucucnucgau

SEQ ID NO: 618 - DNA, 30 bp, Artificial Sequence
  Synthetic
  misc_feature (26)..(26): n is the nucleobase hypoxanthine
ccccagcagcaucacucccuuuctcnucga

SEQ ID NO: 619 - DNA, 32 bp, Artificial Sequence
  Synthetic
gccccagcagcaucacucccuuuctcgucgau

SEQ ID NO: 620 - DNA, 32 bp, Artificial Sequence
  Synthetic
  misc_feature (27)..(27): n is the nucleobase hypoxanthine
gccccagcagcaucacucccuuuctcnucgau

SEQ ID NO: 621 - RNA, 30 nt, Artificial Sequence
  Synthetic
ccccagcagcaucacucccuuucucgucga

SEQ ID NO: 622 - RNA, 30 nt, Artificial Sequence
  Synthetic
  misc_feature (26)..(26): n is the nucleobase hypoxanthine
ccccagcagcaucacucccuuucucnucga

SEQ ID NO: 623 - DNA, 32 bp, Artificial Sequence; identical to SEQ ID NO: 619
  Synthetic

SEQ ID NO: 624 - DNA, 30 bp, Artificial Sequence; identical to SEQ ID NO: 616
  Synthetic

SEQ ID NO: 625 - RNA, 30 nt, Artificial Sequence; identical to SEQ ID NO: 139
  Synthetic

SEQ ID NO: 626 - RNA, 30 nt, Artificial Sequence; identical to SEQ ID NO: 139
  Synthetic

SEQ ID NO: 627 - RNA, 30 nt, Artificial Sequence
  Synthetic
ccaaccagaaauuggcacaaaugccacugu

SEQ ID NO: 628 - RNA, 30 nt, Artificial Sequence; identical to SEQ ID NO: 627
  Synthetic

SEQ ID NO: 629 - RNA, 30 nt, Artificial Sequence
  Synthetic
ugaggcgaagcauucuuucuauuccaucuc

SEQ ID NO: 630 - RNA, 30 nt, Artificial Sequence; identical to SEQ ID NO: 629
  Synthetic

SEQ ID NO: 631 - RNA, 30 nt, Artificial Sequence
  Synthetic
cucuguagucuggagcaagaugcccacgca

SEQ ID NO: 632 - RNA, 30 nt, Artificial Sequence; identical to SEQ ID NO: 631
  Synthetic

SEQ ID NO: 633 - RNA, 30 nt, Artificial Sequence
  Synthetic
ugcccugaauuccaacugaccuuccacaga

SEQ ID NO: 634 - RNA, 30 nt, Artificial Sequence; identical to SEQ ID NO: 633
  Synthetic

SEQ ID NO: 635 - RNA, 30 nt, Artificial Sequence
  Synthetic
cagaaggaacaugcugaaaagaaccaaucc

SEQ ID NO: 636 - RNA, 30 nt, Artificial Sequence; identical to SEQ ID NO: 635
  Synthetic

SEQ ID NO: 637 - RNA, 30 nt, Artificial Sequence
  Synthetic
auccacuguggcacccagauuauccauguu

SEQ ID NO: 638 - RNA, 30 nt, Artificial Sequence; identical to SEQ ID NO: 637
  Synthetic

SEQ ID NO: 639 - RNA, 30 nt, Artificial Sequence
  Synthetic
gcugagauccuuaaagauagcauccauguc

SEQ ID NO: 640 - RNA, 30 nt, Artificial Sequence; identical to SEQ ID NO: 639
  Synthetic

SEQ ID NO: 641 - RNA, 30 nt, Artificial Sequence
  Synthetic
uuaauccaucucuucagauauguccacaga

SEQ ID NO: 642 - RNA, 30 nt, Artificial Sequence; identical to SEQ ID NO: 641
  Synthetic

SEQ ID NO: 643 - RNA, 30 nt, Artificial Sequence; identical to SEQ ID NO: 139
  Synthetic

SEQ ID NO: 644 - RNA, 30 nt, Artificial Sequence; identical to SEQ ID NO: 139
  Synthetic

SEQ ID NO: 645 - RNA, 30 nt, Artificial Sequence; identical to SEQ ID NO: 139
  Synthetic

SEQ ID NO: 646 - RNA, 30 nt, Artificial Sequence; identical to SEQ ID NO: 139
  Synthetic

SEQ ID NO: 647 - RNA, 30 nt, Artificial Sequence; identical to SEQ ID NO: 139
  Synthetic

SEQ ID NO: 648 - RNA, 30 nt, Artificial Sequence; identical to SEQ ID NO: 139
  Synthetic

SEQ ID NO: 649 - RNA, 30 nt, Artificial Sequence; identical to SEQ ID NO: 139
  Synthetic

SEQ ID NO: 650 - RNA, 30 nt, Artificial Sequence; identical to SEQ ID NO: 139
  Synthetic

SEQ ID NO: 651 - RNA, 30 nt, Artificial Sequence; identical to SEQ ID NO: 139
  Synthetic

SEQ ID NO: 652 - RNA, 30 nt, Artificial Sequence; identical to SEQ ID NO: 139
  Synthetic

SEQ ID NO: 653 - RNA, 30 nt, Artificial Sequence; identical to SEQ ID NO: 139
  Synthetic

SEQ ID NO: 654 - RNA, 30 nt, Artificial Sequence; identical to SEQ ID NO: 139
  Synthetic

SEQ ID NO: 655 - RNA, 30 nt, Artificial Sequence; identical to SEQ ID NO: 139
  Synthetic

SEQ ID NO: 656 - RNA, 30 nt, Artificial Sequence; identical to SEQ ID NO: 139
  Synthetic

SEQ ID NO: 657 - RNA, 30 nt, Artificial Sequence; identical to SEQ ID NO: 139
  Synthetic

SEQ ID NO: 658 - RNA, 30 nt, Artificial Sequence; identical to SEQ ID NO: 139
  Synthetic

SEQ ID NO: 659 - RNA, 30 nt, Artificial Sequence; identical to SEQ ID NO: 139
  Synthetic

SEQ ID NO: 660 - RNA, 30 nt, Artificial Sequence; identical to SEQ ID NO: 139
  Synthetic

SEQ ID NO: 661 - RNA, 30 nt, Artificial Sequence; identical to SEQ ID NO: 139
  Synthetic

SEQ ID NO: 662 - RNA, 30 nt, Artificial Sequence; identical to SEQ ID NO: 139
  Synthetic

SEQ ID NO: 663 - RNA, 30 nt, Artificial Sequence; identical to SEQ ID NO: 139
  Synthetic

SEQ ID NO: 664 - RNA, 30 nt, Artificial Sequence; identical to SEQ ID NO: 48
  Synthetic

SEQ ID NO: 665 - RNA, 30 nt, Artificial Sequence; identical to SEQ ID NO: 48
  Synthetic

SEQ ID NO: 666 - RNA, 30 nt, Artificial Sequence; identical to SEQ ID NO: 48
  Synthetic

SEQ ID NO: 667 - RNA, 30 nt, Artificial Sequence; identical to SEQ ID NO: 48
  Synthetic

SEQ ID NO: 668 - RNA, 30 nt, Artificial Sequence; identical to SEQ ID NO: 48
  Synthetic

SEQ ID NO: 669 - RNA, 30 nt, Artificial Sequence; identical to SEQ ID NO: 48
  Synthetic

SEQ ID NO: 670 - RNA, 30 nt, Artificial Sequence; identical to SEQ ID NO: 48
  Synthetic

SEQ ID NO: 671 - RNA, 30 nt, Artificial Sequence; identical to SEQ ID NO: 48
  Synthetic

SEQ ID NO: 672 - RNA, 32 nt, Artificial Sequence
  Synthetic
uacauaauuuacacaaaggcaaugccaucacc

SEQ ID NO: 673 - RNA, 30 nt, Artificial Sequence; identical to SEQ ID NO: 48
  Synthetic

SEQ ID NO: 674 - RNA, 30 nt, Artificial Sequence; identical to SEQ ID NO: 48
  Synthetic

SEQ ID NO: 675 - RNA, 30 nt, Artificial Sequence; identical to SEQ ID NO: 48
  Synthetic

SEQ ID NO: 676 - RNA, 30 nt, Artificial Sequence; identical to SEQ ID NO: 48
  Synthetic

SEQ ID NO: 677 - RNA, 30 nt, Artificial Sequence; identical to SEQ ID NO: 48
  Synthetic

SEQ ID NO: 678 - RNA, 30 nt, Artificial Sequence; identical to SEQ ID NO: 48
  Synthetic

SEQ ID NO: 679 - RNA, 30 nt, Artificial Sequence; identical to SEQ ID NO: 48
  Synthetic

SEQ ID NO: 680 - RNA, 30 nt, Artificial Sequence; identical to SEQ ID NO: 48
  Synthetic

SEQ ID NO: 681 - RNA, 30 nt, Artificial Sequence; identical to SEQ ID NO: 48
  Synthetic

SEQ ID NO: 682 - RNA, 30 nt, Artificial Sequence; identical to SEQ ID NO: 48
  Synthetic

SEQ ID NO: 683 - RNA, 30 nt, Artificial Sequence; identical to SEQ ID NO: 48
  Synthetic

SEQ ID NO: 684 - RNA, 30 nt, Artificial Sequence; identical to SEQ ID NO: 48
  Synthetic

SEQ ID NO: 685 - RNA, 30 nt, Artificial Sequence; identical to SEQ ID NO: 48
  Synthetic

SEQ ID NO: 686 - RNA, 30 nt, Artificial Sequence; identical to SEQ ID NO: 48
  Synthetic

SEQ ID NO: 687 - RNA, 30 nt, Artificial Sequence; identical to SEQ ID NO: 48
  Synthetic

SEQ ID NO: 688 - RNA, 30 nt, Artificial Sequence; identical to SEQ ID NO: 48
  Synthetic

SEQ ID NO: 689 - RNA, 30 nt, Artificial Sequence; identical to SEQ ID NO: 7
  Synthetic

SEQ ID NO: 690 - RNA, 30 nt, Artificial Sequence; identical to SEQ ID NO: 7
  Synthetic

SEQ ID NO: 691 - RNA, 30 nt, Artificial Sequence; identical to SEQ ID NO: 7
  Synthetic

SEQ ID NO: 692 - RNA, 30 nt, Artificial Sequence; identical to SEQ ID NO: 7
  Synthetic

SEQ ID NO: 693 - RNA, 30 nt, Artificial Sequence; identical to SEQ ID NO: 7
  Synthetic

SEQ ID NO: 694 - RNA, 30 nt, Artificial Sequence; identical to SEQ ID NO: 7
  Synthetic

SEQ ID NO: 695 - RNA, 30 nt, Artificial Sequence; identical to SEQ ID NO: 7
  Synthetic

SEQ ID NO: 696 - RNA, 30 nt, Artificial Sequence; identical to SEQ ID NO: 7
  Synthetic

SEQ ID NO: 697 - RNA, 30 nt, Artificial Sequence; identical to SEQ ID NO: 7
  Synthetic

SEQ ID NO: 698 - RNA, 30 nt, Artificial Sequence; identical to SEQ ID NO: 2
  Synthetic

SEQ ID NO: 699 - RNA, 30 nt, Artificial Sequence; identical to SEQ ID NO: 7
  Synthetic

SEQ ID NO: 700 - RNA, 30 nt, Artificial Sequence; identical to SEQ ID NO: 7
  Synthetic

SEQ ID NO: 701 - RNA, 30 nt, Artificial Sequence; identical to SEQ ID NO: 7
  Synthetic

SEQ ID NO: 702 - RNA, 30 nt, Artificial Sequence; identical to SEQ ID NO: 7
  Synthetic

SEQ ID NO: 703 - RNA, 30 nt, Artificial Sequence; identical to SEQ ID NO: 7
  Synthetic

SEQ ID NO: 704 - RNA, 30 nt, Artificial Sequence; identical to SEQ ID NO: 7
  Synthetic

SEQ ID NO: 705 - RNA, 30 nt, Artificial Sequence; identical to SEQ ID NO: 627
  Synthetic

SEQ ID NO: 706 - RNA, 30 nt, Artificial Sequence; identical to SEQ ID NO: 627
  Synthetic

SEQ ID NO: 707 - RNA, 30 nt, Artificial Sequence; identical to SEQ ID NO: 627
  Synthetic

SEQ ID NO: 708 - RNA, 30 nt, Artificial Sequence; identical to SEQ ID NO: 627
  Synthetic

SEQ ID NO: 709 - RNA, 30 nt, Artificial Sequence; identical to SEQ ID NO: 635
  Synthetic

SEQ ID NO: 710 - RNA, 30 nt, Artificial Sequence; identical to SEQ ID NO: 633
  Synthetic

SEQ ID NO: 711 - RNA, 30 nt, Artificial Sequence; identical to SEQ ID NO: 629
  Synthetic

SEQ ID NO: 712 - RNA, 32 nt, Artificial Sequence; identical to SEQ ID NO: 63
  Synthetic

SEQ ID NO: 713 - RNA, 30 nt, Artificial Sequence; identical to SEQ ID NO: 641
  Synthetic

SEQ ID NO: 714 - RNA, 30 nt, Artificial Sequence; identical to SEQ ID NO: 637
  Synthetic

SEQ ID NO: 715 - RNA, 30 nt, Artificial Sequence; identical to SEQ ID NO: 637
  Synthetic

SEQ ID NO: 716 - RNA, 30 nt, Artificial Sequence; identical to SEQ ID NO: 637
  Synthetic

SEQ ID NO: 717 - RNA, 30 nt, Artificial Sequence; identical to SEQ ID NO: 637
  Synthetic

SEQ ID NO: 718 - RNA, 30 nt, Artificial Sequence; identical to SEQ ID NO: 7
  Synthetic

SEQ ID NO: 719 - RNA, 30 nt, Artificial Sequence; identical to SEQ ID NO: 7
  Synthetic

SEQ ID NO: 720 - RNA, 32 nt, Artificial Sequence; identical to SEQ ID NO: 63
  Synthetic

SEQ ID NO: 721 - RNA, 31 nt, Artificial Sequence; identical to SEQ ID NO: 138
  Synthetic

SEQ ID NO: 722 - RNA, 30 nt, Artificial Sequence; identical to SEQ ID NO: 139
  Synthetic

SEQ ID NO: 723 - RNA, 30 nt, Artificial Sequence; identical to SEQ ID NO: 139
  Synthetic

SEQ ID NO: 724 - RNA, 30 nt, Artificial Sequence; identical to SEQ ID NO: 139
  Synthetic

SEQ ID NO: 725 - RNA, 30 nt, Artificial Sequence; identical to SEQ ID NO: 430
  Synthetic

SEQ ID NO: 726 - DNA, 30 bp, Artificial Sequence
  Synthetic
cucuuuccauggaagguucuaaactauccu

SEQ ID NO: 727 - RNA, 30 nt, Artificial Sequence
  Synthetic
  misc_feature (25)..(25): n is the nucleobase associated with the zdnp nucleoside
cucuuuccauggaagguucuaaacnauccu

SEQ ID NO: 728 - RNA, 30 nt, Artificial Sequence; identical to SEQ ID NO: 727
  Synthetic
  misc_feature (25)..(25): n is the nucleobase associated with the zdnp nucleoside

SEQ ID NO: 729 - RNA, 32 nt, Artificial Sequence; identical to SEQ ID NO: 435
  Synthetic

SEQ ID NO: 730 - RNA, 30 nt, Artificial Sequence; identical to SEQ ID NO: 441
  Synthetic

SEQ ID NO: 731 - RNA, 32 nt, Artificial Sequence; identical to SEQ ID NO: 398
  Synthetic

SEQ ID NO: 732 - RNA, 32 nt, Artificial Sequence; identical to SEQ ID NO: 398
  Synthetic

SEQ ID NO: 733 - RNA, 32 nt, Artificial Sequence; identical to SEQ ID NO: 398
  Synthetic

SEQ ID NO: 734 - RNA, 32 nt, Artificial Sequence; identical to SEQ ID NO: 435
  Synthetic

SEQ ID NO: 735 - RNA, 31 nt, Artificial Sequence
  Synthetic
ucucuuuccauggaagguucuaaacauccug

SEQ ID NO: 736 - RNA, 30 nt, Artificial Sequence; identical to SEQ ID NO: 430
  Synthetic

SEQ ID NO: 737 - RNA, 30 nt, Artificial Sequence; identical to SEQ ID NO: 430
  Synthetic

SEQ ID NO: 738 - RNA, 30 nt, Artificial Sequence; identical to SEQ ID NO: 430
  Synthetic

SEQ ID NO: 739 - RNA, 30 nt, Artificial Sequence; identical to SEQ ID NO: 441
  Synthetic

SEQ ID NO: 740 - RNA, 29 nt, Artificial Sequence
  Synthetic
cucuuuccauggaagguucuaaacauccu

SEQ ID NO: 741 - RNA, 29 nt, Artificial Sequence
  Synthetic
cucuuucauggaagguucuaaaccauccu

SEQ ID NO: 742 - RNA, 29 nt, Artificial Sequence
  Synthetic
cucuuuccuggaagguucuaaaccauccu

SEQ ID NO: 743 - RNA, 29 nt, Artificial Sequence
  Synthetic
cucuuuccaggaagguucuaaaccauccu

SEQ ID NO: 744 - RNA, 29 nt, Artificial Sequence
  Synthetic
cucuuuccaugaagguucuaaaccauccu

SEQ ID NO: 745 - RNA, 29 nt, Artificial Sequence
  Synthetic
cucuuuccaugaagguucuaaaccauccu

SEQ ID NO: 746 - RNA, 29 nt, Artificial Sequence
  Synthetic
cucuuuccauggagguucuaaaccauccu

SEQ ID NO: 747 - RNA, 29 nt, Artificial Sequence
  Synthetic
cucuuuccauggagguucuaaaccauccu

SEQ ID NO: 748 - RNA, 29 nt, Artificial Sequence
  Synthetic
cucuuuccauggaaguucuaaaccauccu

SEQ ID NO: 749 - RNA, 29 nt, Artificial Sequence
  Synthetic
cucuuuccauggaaguucuaaaccauccu

SEQ ID NO: 750 - RNA, 29 nt, Artificial Sequence
  Synthetic
cucuuuccauggaaggucuaaaccauccu

SEQ ID NO: 751 - RNA, 29 nt, Artificial Sequence
  Synthetic
cucuuuccauggaaggucuaaaccauccu

SEQ ID NO: 752 - RNA, 29 nt, Artificial Sequence
  Synthetic
cucuuuccauggaagguuuaaaccauccu

SEQ ID NO: 753 - RNA, 28 nt, Artificial Sequence
  Synthetic
cucuuuccagaagguucuaaaccauccu

SEQ ID NO: 754 - RNA, 28 nt, Artificial Sequence
  Synthetic
cucuuuccauaagguucuaaaccauccu

SEQ ID NO: 755 - RNA, 27 nt, Artificial Sequence
  Synthetic
cucuuuccaaagguucuaaaccauccu

SEQ ID NO: 756 - RNA, 27 nt, Artificial Sequence
  Synthetic
cucuuuccauggguucuaaaccauccu

SEQ ID NO: 757 - RNA, 28 nt, Artificial Sequence
  Synthetic
cucuuuccauggaauucuaaaccauccu

SEQ ID NO: 758 - RNA, 28 nt, Artificial Sequence
  Synthetic
cucuuuccauggaagucuaaaccauccu

SEQ ID NO: 759 - RNA, 27 nt, Artificial Sequence
  Synthetic
cucuuuccauggagucuaaaccauccu

SEQ ID NO: 760 - RNA, 27 nt, Artificial Sequence
  Synthetic
cucuuuccaaagguucuaaaccauccu

SEQ ID NO: 761 - RNA, 27 nt, Artificial Sequence
  Synthetic
cucuuuccauggguucuaaaccauccu

SEQ ID NO: 762 - RNA, 27 nt, Artificial Sequence
  Synthetic
cucuuuccauggauucuaaaccauccu

SEQ ID NO: 763 - RNA, 24 nt, Artificial Sequence
  Synthetic
cucuuuccaaaucuaaaccauccu

SEQ ID NO: 764 - RNA, 27 nt, Artificial Sequence
  Synthetic
cucuuuccaaagguucuaaaccauccu

SEQ ID NO: 765 - RNA, 27 nt, Artificial Sequence
  Synthetic
cucuuuccauggguucuaaaccauccu

SEQ ID NO: 766 - RNA, 27 nt, Artificial Sequence
  Synthetic
cucuuuccauggauucuaaaccauccu

SEQ ID NO: 767 - RNA, 24 nt, Artificial Sequence
  Synthetic
cucuuuccaaaucuaaaccauccu

SEQ ID NO: 768 - RNA, 30 nt, Artificial Sequence; identical to SEQ ID NO: 622
  Synthetic
  misc_feature (26)..(26): n is the nucleobase hypoxanthine

SEQ ID NO: 769 - DNA, 30 bp, Artificial Sequence; identical to SEQ ID NO: 618
  Synthetic
  misc_feature (26)..(26): n is the nucleobase hypoxanthine

SEQ ID NO: 770 - DNA, 30 bp, Artificial Sequence
  Synthetic
  misc_feature (26)..(26): n is the nucleobase hypoxanthine
ccccagcagcuucagucccuuuctcnucga

SEQ ID NO: 771 - DNA, 30 bp, Artificial Sequence; identical to SEQ ID NO: 618
  Synthetic
  misc_feature (26)..(26): n is the nucleobase hypoxanthine

SEQ ID NO: 772 - DNA, 30 bp, Artificial Sequence; identical to SEQ ID NO: 770
  Synthetic
  misc_feature (26)..(26): n is the nucleobase hypoxanthine

SEQ ID NO: 773 - DNA, 30 bp, Artificial Sequence; identical to SEQ ID NO: 616
  Synthetic

SEQ ID NO: 774 - DNA, 30 bp, Artificial Sequence
  Synthetic
ccccagcagcuucagucccuuuctcgucga

SEQ ID NO: 775 - DNA, 30 bp, Artificial Sequence
  Synthetic
  misc_feature (26)..(26): n is the nucleobase hypoxanthine
ccccagcagcaucacucccuuuctanucga

SEQ ID NO: 776 - DNA, 30 bp, Artificial Sequence
  Synthetic
  misc_feature (26)..(26): n is the nucleobase hypoxanthine
ccccagcagcuucagucccuuuctanucga

SEQ ID NO: 777 - DNA, 30 bp, Artificial Sequence; identical to SEQ ID NO: 775
  Synthetic
  misc_feature (26)..(26): n is the nucleobase hypoxanthine

SEQ ID NO: 778 - DNA, 30 bp, Artificial Sequence; identical to SEQ ID NO: 776
  Synthetic
  misc_feature (26)..(26): n is the nucleobase hypoxanthine

SEQ ID NO: 779 - DNA, 30 bp, Artificial Sequence; identical to SEQ ID NO: 775
  Synthetic
  misc_feature (26)..(26): n is the nucleobase hypoxanthine

SEQ ID NO: 780 - DNA, 30 bp, Artificial Sequence; identical to SEQ ID NO: 776
  Synthetic
  misc_feature (26)..(26): n is the nucleobase hypoxanthine

SEQ ID NO: 781 - DNA, 30 bp, Artificial Sequence
  Synthetic
  misc_feature (26)..(26): n is the nucleobase hypoxanthine
ccccagcagcaucacucccuuuctunucga

SEQ ID NO: 782 - DNA, 30 bp, Artificial Sequence
  Synthetic
  misc_feature (26)..(26): n is the nucleobase hypoxanthine
ccccagcagcuucagucccuuuctunucga

SEQ ID NO: 783 - DNA, 30 bp, Artificial Sequence; identical to SEQ ID NO: 775
  Synthetic
  misc_feature (26)..(26): n is the nucleobase hypoxanthine

SEQ ID NO: 784 - DNA, 30 bp, Artificial Sequence; identical to SEQ ID NO: 776
  Synthetic
  misc_feature (26)..(26): n is the nucleobase hypoxanthine

SEQ ID NO: 785 - DNA, 30 bp, Artificial Sequence; identical to SEQ ID NO: 775
  Synthetic
  misc_feature (26)..(26): n is the nucleobase hypoxanthine

SEQ ID NO: 786 - DNA, 30 bp, Artificial Sequence; identical to SEQ ID NO: 776
  Synthetic
  misc_feature (26)..(26): n is the nucleobase hypoxanthine

SEQ ID NO: 787 - DNA, 30 bp, Artificial Sequence; identical to SEQ ID NO: 618
  Synthetic
  misc_feature (26)..(26): n is the nucleobase hypoxanthine

SEQ ID NO: 788 - DNA, 30 bp, Artificial Sequence; identical to SEQ ID NO: 770
  Synthetic
  misc_feature (26)..(26): n is the nucleobase hypoxanthine

SEQ ID NO: 789 - DNA, 30 bp, Artificial Sequence; identical to SEQ ID NO: 770
  Synthetic
  misc_feature (26)..(26): n is the nucleobase hypoxanthine

SEQ ID NO: 790 - DNA, 30 bp, Artificial Sequence; identical to SEQ ID NO: 776
  Synthetic
  misc_feature (26)..(26): n is the nucleobase hypoxanthine

SEQ ID NO: 791 - DNA, 30 bp, Artificial Sequence; identical to SEQ ID NO: 618
  Synthetic
  misc_feature (26)..(26): n is the nucleobase hypoxanthine

SEQ ID NO: 792 - DNA, 30 bp, Artificial Sequence; identical to SEQ ID NO: 770
  Synthetic
  misc_feature (26)..(26): n is the nucleobase hypoxanthine

SEQ ID NO: 793 - DNA, 30 bp, Artificial Sequence; identical to SEQ ID NO: 770
  Synthetic
  misc_feature (26)..(26): n is the nucleobase hypoxanthine

SEQ ID NO: 794 - DNA, 30 bp, Artificial Sequence; identical to SEQ ID NO: 776
  Synthetic
  misc_feature (26)..(26): n is the nucleobase hypoxanthine

SEQ ID NO: 795 - DNA, 30 bp, Artificial Sequence; identical to SEQ ID NO: 770
  Synthetic
  misc_feature (26)..(26): n is the nucleobase hypoxanthine

SEQ ID NO: 796 - DNA, 30 bp, Artificial Sequence; identical to SEQ ID NO: 770
  Synthetic
  misc_feature (26)..(26): n is the nucleobase hypoxanthine

SEQ ID NO: 797 - DNA, 30 bp, Artificial Sequence; identical to SEQ ID NO: 776
  Synthetic
  misc_feature (26)..(26): n is the nucleobase hypoxanthine

SEQ ID NO: 798 - DNA, 30 bp, Artificial Sequence; identical to SEQ ID NO: 770
  Synthetic
  misc_feature (26)..(26): n is the nucleobase hypoxanthine

SEQ ID NO: 799 - DNA, 30 bp, Artificial Sequence; identical to SEQ ID NO: 770
  Synthetic
  misc_feature (26)..(26): n is the nucleobase hypoxanthine

SEQ ID NO: 800 - DNA, 30 bp, Artificial Sequence
  Synthetic
ccccagcagcuucagucccuuuctugucga

SEQ ID NO: 801 - RNA, 30 nt, Artificial Sequence
  Synthetic
  misc_feature (26)..(26): n is the nucleobase hypoxanthine
ucagcacagccuuaugcacggccucngaga

SEQ ID NO: 802 - RNA, 30 nt, Artificial Sequence
  Synthetic
  misc_feature (26)..(26): n is the nucleobase hypoxanthine
ucgucgauggucagcacagccuuacncacg

SEQ ID NO: 803 - RNA, 30 nt, Artificial Sequence
  Synthetic
  misc_feature (26)..(26): n is the nucleobase hypoxanthine
cagcuucagucccuuucucgucgacnguca

SEQ ID NO: 804 - RNA, 30 nt, Artificial Sequence; identical to SEQ ID NO: 802
  Synthetic
  misc_feature (26)..(26): n is the nucleobase hypoxanthine

SEQ ID NO: 805 - RNA, 30 nt, Artificial Sequence
  Synthetic
  misc_feature (27)..(27): n is the nucleobase hypoxanthine
cucgucgauggucagcacagccuuacncac

SEQ ID NO: 806 - RNA, 30 nt, Artificial Sequence
  Synthetic
  misc_feature (24)..(24): n is the nucleobase hypoxanthine
gucgauggucagcacagccuuacncacggc

SEQ ID NO: 807 - RNA, 30 nt, Artificial Sequence
  Synthetic
  misc_feature (23)..(23): n is the nucleobase hypoxanthine
ucgauggucagcacagccuuacncacggcc

SEQ ID NO: 808 - RNA, 30 nt, Artificial Sequence; identical to SEQ ID NO: 802
  Synthetic
  misc_feature (26)..(26): n is the nucleobase hypoxanthine

SEQ ID NO: 809 - RNA, 30 nt, Artificial Sequence; identical to SEQ ID NO: 802
  Synthetic
  misc_feature (26)..(26): n is the nucleobase hypoxanthine

SEQ ID NO: 810 - RNA, 30 nt, Artificial Sequence; identical to SEQ ID NO: 802
  Synthetic
  misc_feature (26)..(26): n is the nucleobase hypoxanthine

SEQ ID NO: 811 - RNA, 30 nt, Artificial Sequence
  Synthetic
ucucgucgauggucagcacagccucaugca

SEQ ID NO: 812 - RNA, 30 nt, Artificial Sequence
  Synthetic
uucucgucgauggucagcacagccucaugc

SEQ ID NO: 813 - RNA, 30 nt, Artificial Sequence
  Synthetic
cucgucgauggucagcacagccucaugcac

SEQ ID NO: 814 - RNA, 30 nt, Artificial Sequence
  Synthetic
ucgucgauggucagcacagccucaugcacg

SEQ ID NO: 815 - RNA, 30 nt, Artificial Sequence
  Synthetic
cgucgauggucagcacagccucaugcacgg

SEQ ID NO: 816 - RNA, 30 nt, Artificial Sequence; identical to SEQ ID NO: 811
  Synthetic

SEQ ID NO: 817 - RNA, 30 nt, Artificial Sequence; identical to SEQ ID NO: 811
  Synthetic

SEQ ID NO: 818 - RNA, 30 nt, Artificial Sequence; identical to SEQ ID NO: 811
  Synthetic

SEQ ID NO: 819 - RNA, 30 nt, Artificial Sequence; identical to SEQ ID NO: 801
  Synthetic
  misc_feature (26)..(26): n is the nucleobase hypoxanthine

SEQ ID NO: 820 - RNA, 30 nt, Artificial Sequence
  Synthetic
  misc_feature (27)..(27): n is the nucleobase hypoxanthine
gucagcacagccuuaugcacggccucngag

SEQ ID NO: 821 - RNA, 30 nt, Artificial Sequence
  Synthetic
  misc_feature (25)..(25): n is the nucleobase hypoxanthine
cagcacagccuuaugcacggccucngagag

SEQ ID NO: 822 - RNA, 30 nt, Artificial Sequence
  Synthetic
  misc_feature (24)..(24): n is the nucleobase hypoxanthine
agcacagccuuaugcacggccucngagagc

SEQ ID NO: 823 - RNA, 30 nt, Artificial Sequence
  Synthetic
  misc_feature (23)..(23): n is the nucleobase hypoxanthine
gcacagccuuaugcacggccucngagagcu

SEQ ID NO: 824 - RNA, 30 nt, Artificial Sequence; identical to SEQ ID NO: 801
  Synthetic
  misc_feature (26)..(26): n is the nucleobase hypoxanthine

SEQ ID NO: 825 - RNA, 30 nt, Artificial Sequence; identical to SEQ ID NO: 801
  Synthetic
  misc_feature (26)..(26): n is the nucleobase hypoxanthine

SEQ ID NO: 826 - RNA, 30 nt, Artificial Sequence; identical to SEQ ID NO: 801
  Synthetic
  misc_feature (26)..(26): n is the nucleobase hypoxanthine

SEQ ID NO: 827 - RNA, 30 nt, Artificial Sequence; identical to SEQ ID NO: 803
  Synthetic
  misc_feature (26)..(26): n is the nucleobase hypoxanthine

SEQ ID NO: 828 - RNA, 30 nt, Artificial Sequence
  Synthetic
  misc_feature (27)..(27): n is the nucleobase hypoxanthine
gcagcuucagucccuuucucgucgacnguc

SEQ ID NO: 829 - RNA, 30 nt, Artificial Sequence
  Synthetic
  misc_feature (25)..(25): n is the nucleobase hypoxanthine
agcuucagucccuuucucgucgacngucag

SEQ ID NO: 830 - RNA, 30 nt, Artificial Sequence
  Synthetic
  misc_feature (24)..(24): n is the nucleobase hypoxanthine
gcuucagucccuuucucgucgacngucagc

SEQ ID NO: 831 - RNA, 30 nt, Artificial Sequence
  Synthetic
  misc_feature (23)..(23): n is the nucleobase hypoxanthine
cuucagucccuuucucgucgacngucagca

SEQ ID NO: 832 - RNA, 30 nt, Artificial Sequence; identical to SEQ ID NO: 803
  Synthetic
  misc_feature (26)..(26): n is the nucleobase hypoxanthine

SEQ ID NO: 833 - RNA, 30 nt, Artificial Sequence; identical to SEQ ID NO: 803
  Synthetic
  misc_feature (26)..(26): n is the nucleobase hypoxanthine

SEQ ID NO: 834 - RNA, 30 nt, Artificial Sequence; identical to SEQ ID NO: 803
  Synthetic
  misc_feature (26)..(26): n is the nucleobase hypoxanthine

SEQ ID NO: 835 - RNA, 30 nt, Artificial Sequence; identical to SEQ ID NO: 802
  Synthetic
  misc_feature (26)..(26): n is the nucleobase hypoxanthine

SEQ ID NO: 836 - DNA, 30 bp, Artificial Sequence
  Synthetic
ucucgucgauggucagcacagcctcaugca

SEQ ID NO: 837 - DNA, 30 bp, Artificial Sequence
  Synthetic
  misc_feature (26)..(26): n is the nucleobase hypoxanthine
ucagcacagccuuaugcacggcctcngaga

SEQ ID NO: 838 - RNA, 30 nt, Artificial Sequence; identical to SEQ ID NO: 803
  Synthetic
  misc_feature (26)..(26): n is the nucleobase hypoxanthine

SEQ ID NO: 839 - RNA, 30 nt, Artificial Sequence; identical to SEQ ID NO: 802
  Synthetic
  misc_feature (26)..(26): n is the nucleobase hypoxanthine

SEQ ID NO: 840 - RNA, 30 nt, Artificial Sequence; identical to SEQ ID NO: 802
  Synthetic
  misc_feature (26)..(26): n is the nucleobase hypoxanthine

SEQ ID NO: 841 - RNA, 30 nt, Artificial Sequence; identical to SEQ ID NO: 802
  Synthetic
  misc_feature (26)..(26): n is the nucleobase hypoxanthine

SEQ ID NO: 842 - RNA, 30 nt, Artificial Sequence; identical to SEQ ID NO: 802
  Synthetic
  misc_feature (26)..(26): n is the nucleobase hypoxanthine

SEQ ID NO: 843 - RNA, 30 nt, Artificial Sequence; identical to SEQ ID NO: 802
  Synthetic
  misc_feature (26)..(26): n is the nucleobase hypoxanthine

SEQ ID NO: 844 - RNA, 30 nt, Artificial Sequence; identical to SEQ ID NO: 802
  Synthetic
  misc_feature (26)..(26): n is the nucleobase hypoxanthine

SEQ ID NO: 845 - RNA, 30 nt, Artificial Sequence; identical to SEQ ID NO: 802
  Synthetic
  misc_feature (26)..(26): n is the nucleobase hypoxanthine

SEQ ID NO: 846 - RNA, 30 nt, Artificial Sequence; identical to SEQ ID NO: 802
  Synthetic
  misc_feature (26)..(26): n is the nucleobase hypoxanthine

SEQ ID NO: 847 - RNA, 31 nt, Artificial Sequence
  Synthetic
  misc_feature (26)..(26): n is the nucleobase hypoxanthine
ucgucgauggucagcacagccuuacncacgg

SEQ ID NO: 848 - RNA, 31 nt, Artificial Sequence; identical to SEQ ID NO: 847
  Synthetic
  misc_feature (26)..(26): n is the nucleobase hypoxanthine

SEQ ID NO: 849 - RNA, 32 nt, Artificial Sequence
  Synthetic
  misc_feature (27)..(27): n is the nucleobase hypoxanthine
cucgucgauggucagcacagccuuacncacgg

SEQ ID NO: 850 - RNA, 32 nt, Artificial Sequence; identical to SEQ ID NO: 849
  Synthetic
  misc_feature (27)..(27): n is the nucleobase hypoxanthine

SEQ ID NO: 851 - RNA, 30 nt, Artificial Sequence; identical to SEQ ID NO: 811
  Synthetic

SEQ ID NO: 852 - RNA, 30 nt, Artificial Sequence; identical to SEQ ID NO: 811
  Synthetic

SEQ ID NO: 853 - RNA, 30 nt, Artificial Sequence; identical to SEQ ID NO: 811
  Synthetic

SEQ ID NO: 854 - RNA, 30 nt, Artificial Sequence; identical to SEQ ID NO: 811
  Synthetic

SEQ ID NO: 855 - RNA, 30 nt, Artificial Sequence; identical to SEQ ID NO: 811
  Synthetic

SEQ ID NO: 856 - RNA, 30 nt, Artificial Sequence; identical to SEQ ID NO: 811
  Synthetic

SEQ ID NO: 857 - RNA, 30 nt, Artificial Sequence; identical to SEQ ID NO: 811
  Synthetic

SEQ ID NO: 858 - RNA, 30 nt, Artificial Sequence; identical to SEQ ID NO: 811
  Synthetic

SEQ ID NO: 859 - RNA, 30 nt, Artificial Sequence; identical to SEQ ID NO: 811
  Synthetic

SEQ ID NO: 860 - RNA, 31 nt, Artificial Sequence
  Synthetic
ucucgucgauggucagcacagccucaugcac

SEQ ID NO: 861 - RNA, 31 nt, Artificial Sequence; identical to SEQ ID NO: 860
  Synthetic

SEQ ID NO: 862 - RNA, 31 nt, Artificial Sequence; identical to SEQ ID NO: 860
  Synthetic

SEQ ID NO: 863 - RNA, 32 nt, Artificial Sequence
  Synthetic
uucucgucgauggucagcacagccucaugcac

SEQ ID NO: 864 - RNA, 32 nt, Artificial Sequence; identical to SEQ ID NO: 863
  Synthetic

SEQ ID NO: 865 - RNA, 30 nt, Artificial Sequence
  Synthetic
  misc_feature (26)..(26): n is the nucleobase hypoxanthine
ucaucgauggucagcacagccuuacncacg

SEQ ID NO: 866 - RNA, 30 nt, Artificial Sequence
  Synthetic
ucucaucgauggucagcacagccucaugca

SEQ ID NO: 867 - RNA, 30 nt, Artificial Sequence; identical to SEQ ID NO: 866
  Synthetic

SEQ ID NO: 868 - RNA, 30 nt, Artificial Sequence; identical to SEQ ID NO: 802
  Synthetic
  misc_feature (26)..(26): n is the nucleobase hypoxanthine

SEQ ID NO: 869 - RNA, 30 nt, Artificial Sequence; identical to SEQ ID NO: 637
  Synthetic

SEQ ID NO: 870 - DNA, 21 bp, Artificial Sequence
  Synthetic
gccucgcgggcgcaaugaatt

SEQ ID NO: 871 - DNA, 21 bp, Artificial Sequence
  Synthetic
uucauugcgcccgcgaggcat

SEQ ID NO: 872 - DNA, 21 bp, Artificial Sequence
  Synthetic
cgagcatcccccaaagttcac

SEQ ID NO: 873 - DNA, 22 bp, Artificial Sequence
  Synthetic
cactcccagggagaccaaaagc

SEQ ID NO: 874 - DNA, 23 bp, Artificial Sequence
  Synthetic
ggtgttggtgaatttgaagctgg

SEQ ID NO: 875 - DNA, 21 bp, Artificial Sequence
  Synthetic
gtcagttggacgagttggtgg

SEQ ID NO: 876 - DNA, 21 bp, Artificial Sequence
  Synthetic
cccctacacagcatacgtagg

SEQ ID NO: 877 - DNA, 23 bp, Artificial Sequence
  Synthetic
ccttttctgaatccaaagccacc

SEQ ID NO: 878 - DNA, 22 bp, Artificial Sequence
  Synthetic
cgctgatcagagacatgcttcg

SEQ ID NO: 879 - DNA, 23 bp, Artificial Sequence
  Synthetic
cttcttcatctgttcccacatcc

SEQ ID NO: 880 - DNA, 23 bp, Artificial Sequence
  Synthetic
tcctaacatctacattcccttcg

SEQ ID NO: 881 - DNA, 23 bp, Artificial Sequence
  Synthetic
ctaagcactgtgacaaattagcc

SEQ ID NO: 882 - DNA, 20 bp, Artificial Sequence
  Synthetic
acagaagcttggtaccctcc

SEQ ID NO: 883 - DNA, 20 bp, Artificial Sequence
  Synthetic
tgtgcttttggcacttgagc

SEQ ID NO: 884 - DNA, 21 bp, Artificial Sequence
  Synthetic
cctccagggcatggagatatc

SEQ ID NO: 885 - DNA, 23 bp, Artificial Sequence
  Synthetic
ccatgtcaatggcattctgttcc

SEQ ID NO: 886 - DNA, 20 bp, Artificial Sequence
  Synthetic
caccgagccaccatacagtc

SEQ ID NO: 887 - DNA, 20 bp, Artificial Sequence
  Synthetic
gaacaccagtctccactcgg

SEQ ID NO: 888 - DNA, 23 bp, Artificial Sequence
  Synthetic
ctacattcccctcatgtctttgg

SEQ ID NO: 889 - DNA, 23 bp, Artificial Sequence
  Synthetic
gatctgatcctcctcaatcctcc

SEQ ID NO: 890 - DNA, 1812 bp, Artificial Sequence
  Synthetic
ggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgaggcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctc

SEQ ID NO: 891 - DNA, 118 bp, Artificial Sequence
  Synthetic
gtgacctgcacgtctagggcgcagtagtccagggtttccttgatgatgtcatacttatcctgtcccttttttttccacagctcgcggttgaggacaaactcttcgcggtctttccagt

SEQ ID NO: 892 - DNA, 2796 bp, Homo sapiens
  misc_feature: human ADAR1 p110
atggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtaaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatag

SEQ ID NO: 893 - DNA, 3681 bp, Homo sapiens
  misc_feature: human ADAR1 p150
atgaatccgcggcaggggtattccctcagcggatactacacccatccatttcaaggctatgagcacagacagctcagataccagcagcctgggccaggatcttcccccagtagtttcctgcttaagcaaatagaatttctcaaggggcagctcccagaagcaccggtgattggaaagcagacaccgtcactgccaccttccctcccaggactccggccaaggtttccagtactacttgcctccagtaccagaggcaggcaagtggacatcaggggtgtccccaggggcgtgcatctcggaagtcaggggctccagagagggttccagcatccttcaccacgtggcaggagtctgccacagagaggtgttgattgcctttcctcacatttccaggaactgagtatctaccaagatcaggaacaaaggatcttaaagttcctggaagagcttggggaagggaaggccaccacagcacatgatctgtctgggaaacttgggactccgaagaaagaaatcaatcgagttttatactccctggcaaagaagggcaagctacagaaagaggcaggaacaccccctttgtggaaaatcgcggtctccactcaggcttggaaccagcacagcggagtggtaagaccagacggtcatagccaaggagccccaaactcagacccgagtttggaaccggaagacagaaactccacatctgtctcagaagatcttcttgagccttttattgcagtctcagctcaggcttggaaccagcacagcggagtggtaagaccagacagtcatagccaaggatccccaaactcagacccaggtttggaacctgaagacagcaactccacatctgccttggaagatcctcttgagtttttagacatggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtgaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagtatag

SEQ ID NO: 894 - DNA, 589 bp, Artificial Sequence
  Synthetic
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 895 - DNA, 208 bp, Artificial Sequence
  Synthetic
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatggcaggcatgctgggga

SEQ ID NO: 896 - DNA, 1779 bp, Artificial Sequence
  Synthetic
cggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagact

(19) United States
(12) Patent Application Publication
(10) Pub. No.: US 2023/0220384 A1
(43) Pub. Date: Jul. 13, 2023

(54) OLIGONUCLEOTIDE COMPOSITIONS AND METHODS OF USE THEREOF

(71) Applicants: Prashant MONIAN, Cambridge, CA (US); Chikdu Shakti SHIVAILA, Cambridge, MA (US); Subramanian MARAPPAN, Cambridge, MA (US); Chandra VARGEESE, Cambridge, MA (US); Pachamuthu KANDASAMY, Cambridge, MA (US); Genliang LU, Cambridge, CA (US); Hui YU, Cambridge, MA (US); David Charles Donnell BUTLER, Cambridge, MA (US); Luciano Henrique APPONI, Cambridge, MA (US); Mamoru SHIMIZU, Cambridge, MA (US); Stephany Michelle STANDLEY, Cambridge, MA (US); David John BOULAY, Cambridge, MA (US); Andrew Guzior HOSS, Cambridge, MA (US); Jigar DESAI, Cambridge, MA (US); Jack David GODFREY, Cambridge, MA (US); Hailin YANG, Cambridge, MA (US); Naoki IWAMOTO, Cambridge, MA (US); WAVE LIFE SCIENCES LTD., Singapore (SG)

(72) Inventors: Prashant Monian, Arlington, MA (US); Chikdu Shakti Shivalila, Woburn, MA (US); Subramanian Marappan, Acton, MA (US); Chandra Vargeese, Schwenksville, PA (US); Pachamuthu Kandasamy, Lexington, MA (US); Genliang Lu, Winchester, MA (US); Hui Yu, Arlington, MA (US); David Charles Donnell Butler, Medford, MA (US); Luciano Henrique Apponi, Lynn, MA (US); Mamoru Shimizu, Arlington, MA (US); Stephany Michelle Standley, Wakefield, MA (US); David John Boulay, Cambridge, MA (US); Andrew Guzior Hoss, Cambridge, MA (US); Jigar Desai, Cambridge, MA (US); Jack David Godfrey, Arlington, MA (US); Hailin Yang, West Roxbury, MA (US); Naoki Iwamoto, Boston, MA (US)

(21) Appl. No.: 17/766,677

(22) PCT Filed: Oct. 6, 2020
(86) PCT No.: PCT/US20/54436

§ 371 Date: Apr. 5, 2022

Related U.S. Application Data
(60) Provisional application No. 63/069,696, filed on Aug. 24, 2020.
(60) Provisional application No. 63/022,559, filed on May 10, 2020.
(60) Provisional application No. 62/959,917, filed on Jan. 11, 2020.
(60) Provisional application No. 62/911,334, filed on Oct. 6, 2019.

(51) Int. Cl. C12N 15/113

(52) U.S. Cl. CPC C12N 15/113; C12N 2310/14; C12N 2310/351; C12N 2310/314; C12N 2310/322; C12N 2310/321; C12N 2310/315

(57) ABSTRACT

Among other things, the present disclosure provides oligonucleotides and compositions thereof. In some embodiments, provided oligonucleotides and compositions are useful for adenosine modification. In some embodiments, the present disclosure provides methods for treating various conditions, disorders or diseases that can benefit from adenosine modification.

### CROSS-REFERENCE TO RELATED APPLICATIONS

This application is a National Stage Entry of PCT Application No. PCT/US/2020/054436, filed Oct. 6, 2020 and published on Apr. 15, 2021 as WO/2021/071858, which claims priority to United States Provisional Application Nos. 62/911,334, filed Oct. 6, 2019, 62/959,917, filed Jan. 11, 2020, 63/022,559, filed May 10, 2020, and 63/069,696, filed Aug. 24, 2020, the entirety of each of which is incorporated herein by reference.

### SEQUENCE LISTING

The instant application contains a Sequence Listing, which has been submitted electronically in ASCII format and is hereby incorporated by reference in its entirety. Said ASCII copy, created on Jan. 19, 2023, is named SL.txt and is 186,457 bytes in size.

### BACKGROUND

Oligonucleotides are useful in various applications, e.g., therapeutic, diagnostic, and/or research applications. For example, oligonucleotides targeting various genes can be useful for treatment of conditions, disorders or diseases related to such target genes.

### SUMMARY

Among other things, the present disclosure provides designed oligonucleotides and compositions thereof which oligonucleotides comprise modifications (e.g., modifications to nucleobases sugars, and/or internucleotidic linkages, and patterns thereof) as described herein. In some embodiments, technologies (compounds (e.g., oligonucleotides), compositions, methods, etc.) of the present disclosure (e.g., oligonucleotides, oligonucleotide compositions, methods, etc.) are particularly useful for editing nucleic acids, e.g., site-directed editing in nucleic acids (e.g., editing of target adenosine). In some embodiments, as demonstrated herein, provided technologies can significantly improve efficiency of nucleic acid editing, e.g., modification of one or more A residues, such as conversion of A to I. In some embodiments, the present disclosure provides technologies for editing (e.g., for modifying an A residue, e.g., converting an A to I) in an RNA. In some embodiments, the present disclosure provides technologies for editing (e.g., for modifying an A residue, e.g., converting an A to an I) in a transcript, e.g., mRNA. Among other things, provided technologies provide the benefits of utilization of endogenous proteins such as ADAR (Adenosine Deaminases Acting on RNA) proteins (e.g., ADAR1 and/or ADR2), for editing nucleic acids, e.g., for modifying an A (e.g., as a result of G to A mutation). Those skilled in the art will appreciates that such utilization of endogenous proteins can avoid a number of challenges and/or provide various benefits compared to those technologies that require the delivery of exogenous components (e.g., proteins (e.g., those engineered to bind to oligonucleotides (and/or duplexes thereof with target nucleic acids) to provide desired activities), nucleic acids encoding proteins, viruses, etc.).

Particularly, in some embodiments, oligonucleotides of provided technologies comprise useful sugar modifications and/or patterns thereof (e.g., presence and/or absence of certain modifications), nucleobase modifications and/or patterns thereof (e.g., presence and/or absence of certain modifications), internucleotidic linkages modifications and/or stereochemistry and/or patterns thereof [e.g., types, modifications, and/or configuration (Rp or Sp) of chiral linkage phosphorus, etc.], etc., which, when combined with one or more other structural elements described herein (e.g., additional chemical moieties) can provide high activities and/or various desired properties, e.g., high efficiency of nucleic acid editing, high selectivity, high stability, high cellular uptake, low immune stimulation, low toxicity, improved distribution, improved affinity, etc. In some embodiments, provided oligonucleotides provide high stability, e.g., when compared to oligonucleotides having a high percentage of natural RNA sugars utilized for adenosine editing. In some embodiments, provided oligonucleotides provide high activities, e.g., adenosine editing activity. In some embodiments, provided oligonucleotides provide high selectivity, for example, in some embodiments, provided oligonucleotides provide selective modification of a target adenosine in a target nucleic acid over other adenosine in the same target nucleic acid (e.g., 2, 3, 4, 5, 6, 7, 8, 9, 10, 11, 12, 13, 14, 15, 16, 17, 18, 19, 20 fold or more modification at the target adenosine than another adenosine, or all other adenosine, in a target nucleic acid).

In some embodiments, the present disclosure provides an oligonucleotide comprising a first domain and a second domain, wherein the first domain comprises one or more 2′-F modifications, and the second domain comprises one or more sugars that do not have a 2′-F modification. In some embodiments, a provided oligonucleotide comprises one or more chiral modified internucleotidic linkages. In some embodiments, the present disclosure provides an oligonucleotide comprising:

(a) a first domain; and

(b) a second domain,

wherein the first domain comprises at least 1, 2, 3, 4, 5, 6, 7, 8, 9, 10, 11, 12, 13, 14, 15, 16, 17, 18, 19, or 20 or more sugars comprising a 2′-F modification, or at least 50%, 60%, 70%, 75%, 80%, 85%, 90%, 95%, or 99% of all sugars of the first domain comprises a 2′-F modification;

the second domain comprises at least 1, 2, 3, 4, 5, 6, 7, 8, 9, 10, 11, 12, 13, 14, 15, 16, 17, 18, 19, or 20 or more modified sugars comprising no 2′-F modification, or at least 50%, 60%, 70%, 75%, 80%, 85%, 90%, 95%, or 99% of all sugars of the second domain comprise no 2′-F modification.

In some embodiments, a second domain comprises or consists of a first subdomain, a second subdomain and a third subdomain as described herein.

In some embodiments, a second domain comprises at least 1, 2, 3, 4, 5, 6, 7, 8, 9, 10, 11, 12, 13, 14, 15, 16, 17, 18, 19, or 20 or more modified sugars independently comprising a 2′-OR modification, or at least 50%, 60%, 70%, 75%, 80%, 85%, 90%, 95%, or 99% of all sugars of a second domain comprise a 2′-OR modification, wherein R is optionally substituted C₁₋₆ aliphatic. In some embodiments, R is methyl. In some embodiments, R is CH₂CH₂OCH₃. As described herein, other sugar modifications may also be utilized in accordance with the present disclosure, optionally with base modifications and/or internucleotidic linkage modifications described herein.

In some embodiments, base sequence of a provided oligonucleotide is substantially complementary to the base sequence of a target nucleic acid comprising a target adenosine. In some embodiments, a provided oligonucleotide when aligned to a target nucleic acid comprises one or more mismatches (non-Watson-Crick base pairs). In some embodiments, a provided oligonucleotide when aligned to a target nucleic acid comprises one or more wobbles (e.g., G-U, I-A, G-A, I-U, I-C, etc.). In some embodiments, mismatches and/or wobbles may help one or more proteins, e.g., ADAR1, ADAR2, etc., to recognize a duplex formed by a provided oligonucleotide and a target nucleic acid. In some embodiments, provided oligonucleotides form duplexes with target nucleic acids. In some embodiments, ADAR proteins recognize and bind to such duplexes. In some embodiments, nucleosides opposite to target adenosines are located in the middle of provided oligonucleotides, e.g., with 5-50 nucleosides to 5′ side, and 1-50 nucleosides on its 3′ side. In some embodiments, a 5′ side has more nucleosides than a 3′ side. In some embodiments, a 5′ side has fewer nucleosides than a 3′ side. In some embodiments, a 5′ side has the same number of nucleosides as a 3′ side. In some embodiments, provided oligonucleotides comprise 15-40, e.g., 15, 20, 25, 30, etc. contiguous bases of oligonucleotides described in the Tables. In some embodiments, base sequences of provided oligonucleotides are or comprises base sequences of oligonucleotides described in the Tables.

In some embodiments, with utilization of various structural elements (e.g., various modifications, stereochemistry, and patterns thereof), the present disclosure can achieve desired properties and high activities with short oligonucleotides, e.g., those of about 20-40, 25-40, 25-35, 26-32, 25, 26, 27, 28, 29, 30, 31, 32 33, 34 or 35 nucleobases in length.

In some embodiments, provided oligonucleotides comprise modified nucleobases. In some embodiments, a modified nucleobase promotes modification of a target adenosine. In some embodiments, a nucleobase which is opposite to a target adenine maintains interactions with an enzyme, e.g., ADAR, compared to when a U is present, while interacts with a target adenine less strongly than U (e.g., forming fewer hydrogen bonds). In some embodiments, an opposite nucleobase and/or its associated sugar provide certain flexibility (e.g., when compared to U) to facility modification of a target adenosine by enzymes, e.g., ADAR1, ADAR2, etc. In some embodiments, a nucleobase immediately 5′ or 3′ to the opposite nucleobase (to a target adenine), e.g., I and derivatives thereof, enhances modification of a target adenine. Among other things, the present disclosure recognizes that such a nucleobase may causes less steric hindrance than G when a duplex of a provided oligonucleotide and its target nucleic acid interact with a modifying enzyme, e.g., ADAR1 or ADAR2. In some embodiments, base sequences of oligonucleotides are selected (e.g., when several adenosine residues are suitable targets) and/or designed (e.g., through utilization of various nucleobases described herein) so that steric hindrance may be reduced or removed (e.g., no G next to the opposite nucleoside of a target A).

In some embodiments, oligonucleotides of the present disclosure provides modified internucleotidic linkages (i.e., internucleotidic linkages that are not natural phosphate linkages). In some embodiments, linkage phosphorus of modified internucleotidic linkages (e.g., chiral internucleotidic linkages) are chiral and can exist in different configurations (Rp and Sp). Among other things, the present disclosure demonstrates that incorporation of modified internucleotidic linkage, particularly with control of stereochemistry of linkage phosphorus centers (so that at such a controlled center one configuration is enriched compared to stereorandom oligonucleotide preparation), can significantly improve properties (e.g., stability) and/or activities (e.g., adenosine modifying activities (e.g., converting an adenosine to inosine). In some embodiments, provided oligonucleotides have stereochemical purity significantly higher than stereorandom preparations. In some embodiments, provided oligonucleotides are chirally controlled.

In some embodiments, oligonucleotides of the present disclosure comprise one or more chiral internucleotidic linkages whose linkage phosphorus is chiral (e.g., a phosphorothioate internucleotidic linkage). In some embodiments, at least 1, 2, 3, 4, 5, 6, 7, 8, 9, 10, 11, 12, 13, 14, 15, 16, 17, 18, 19, or 20, or at least 50%, 60%, 70%, 75%, 80%, 85%, 90%, 95%, or 99% of all internucleotidic linkages in an oligonucleotide, are chiral internucleotidic linkages. In some embodiments, at least one internucleotidic linkage is a chiral internucleotidic linkage. In some embodiments, at least one internucleotidic linkage is a natural phosphate linkage. In some embodiments, each internucleotidic linkage is independently a chiral internucleotidic linkage. In some embodiments, at least one chiral internucleotidic linkage is a phosphorothioate internucleotidic linkage. In some embodiments, each is a phosphorothioate internucleotidic linkage. A linkage phosphorus can be either Rp or Sp. In some embodiments, at least one linkage phosphorus is Rp. In some embodiments, at least one linkage phosphorus is Sp. In some embodiments, at least 1, 2, 3, 4, 5, 6, 7, 8, 9, 10, 11, 12, 13, 14, 15, 16, 17, 18, 19, or 20, or at least 50%, 60%, 70%, 75%, 80%, 85%, 90%, 95%, or 99% of all chiral internucleotidic linkages in an oligonucleotide, are Sp. In some embodiments, at least 1, 2, 3, 4, 5, 6, 7, 8, 9, 10, 11, 12, 13, 14, 15, 16, 17, 18, 19, or 20, or at least 50%, 60%, 70%, 75%, 80%, 85%, 90%, 95%, or 99% of all phosphorothioate internucleotidic linkages in an oligonucleotide, are Sp.

In some embodiments, stereochemistry of one or more chiral linkage phosphorus of provided oligonucleotides are controlled in a composition. In some embodiments, the present disclosure provides a composition comprising a plurality of oligonucleotides, wherein oligonucleotides of a plurality share a common base sequence, and the same configuration of linkage phosphorus (e.g., all are Rp or all are Sp for the chiral linkage phosphorus) independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more, or at least 50%, 60%, 70%, 75%, 80%, 85%, 90%, 95%, or 99% of all chiral internucleotidic linkages) chiral internucleotidic linkages (“chirally controlled internucleotidic linkages”). In some embodiments, they share the same stereochemistry at each chiral linkage phosphorus. In some embodiments, oligonucleotides of a plurality share the same constitution. In some embodiments, oligonucleotides of a plurality are structurally identical except the internucleotidic linkages. In some embodiments, oligonucleotides of a plurality are structurally identical. In some embodiments, at least at least about 50%, 60%, 65%, 70%, 75%, 80%, 85%, 90%, or 95% of all oligonucleotides in a composition, or of all oligonucleotides sharing the common base sequence, share the pattern of backbone chiral centers of oligonucleotides of the plurality. In some embodiments, at least about 50%, 60%, 65%, 70%, 75%, 80%, 85%, 90%, or 95% of all oligonucleotides in a composition, or of all oligonucleotides sharing the common base sequence, are oligonucleotides of the plurality.

In some embodiments, the present disclosure provides a chirally controlled oligonucleotide composition of an oligonucleotide, wherein at least about 50%, 60%, 65%, 70%, 75%, 80%, 85%, 90%, or 95% of all oligonucleotides in a composition, or of all oligonucleotides having the same base sequence of the oligonucleotide, or of all oligonucleotide having the same base sequence and sugar and base modifications, or of all oligonucleotides of the same constitution, share the same configuration of linkage phosphorus (e.g., all are Rp or all are Sp for the chiral linkage phosphorus) independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more, or at least 50%, 60%, 70%, 75%, 80%, 85%, 90%, 95%, or 99% of all chiral internucleotidic linkages) chiral internucleotidic linkages with the oligonucleotide. In some embodiments, the present disclosure provides a chirally controlled oligonucleotide composition of an oligonucleotide, wherein at least about 50%, 60%, 65%, 70%, 75%, 80%, 85%, 90%, or 95% of all oligonucleotides in a composition, or of all oligonucleotides having the same base sequence of the oligonucleotide, or of all oligonucleotide having the same base sequence and sugar and base modifications, or of all oligonucleotides of the same constitution, are one or more forms of the oligonucleotide (e.g., acid forms, salt forms (e.g. pharmaceutically acceptable salt forms; as appreciated by those skilled in the art, in case the oligonucleotide is a salt, other salt forms of the corresponding acid or base form of the oligonucleotide), etc.).

In some embodiments, as demonstrated herein chirally controlled oligonucleotide compositions provide a number of advantages, e.g., higher stability, activities, etc., compared to corresponding stereorandom oligonucleotide compositions. In some embodiments, it was observed that chirally controlled oligonucleotide compositions provide high levels of adenosine modifying (e.g., converting A to I) activities with various isoforms of an ADAR protein (e.g., p150 and p110 forms of ADAR1) while corresponding stereorandom compositions provide high levels of adenosine modifying (e.g., converting A to I) activities with only certain isoforms of an ADAR protein (e.g., p150 isoform of ADAR1).

In some embodiments, provided oligonucleotides comprise an additional moiety, e.g., a targeting moiety, a carbohydrate moiety, etc. In some embodiments, an additional moiety is or comprises a ligand for an asialoglycoprotein receptor. In some embodiments, an additional moiety is or comprises GalNAc or derivatives thereof. Among other things, additional moieties may facilitate delivery to certain target locations, e.g., cells, tissues, organs, etc. (e.g., locations comprising receptors that interact with additional moieties). In some embodiments, additional moieties facilitate delivery to liver.

In some embodiments, the present disclosure provides technologies for preparing oligonucleotides and compositions thereof, particularly chirally controlled oligonucleotide compositions. In some embodiments, provided oligonucleotides and compositions thereof are of high purity. In some embodiments, oligonucleotides ofthe present disclosure are at least 50%, 60%, 70%, 75%, 80%, 85%, 90%, 95%, or 99% stereochemically pure at linkage phosphorus of chiral internucleotidic linkages. In some embodiments, oligonucleotides of the present disclosure are prepared stereoselectively and are substantially free of stereoisomers. In some embodiments, in provided compositions comprising a plurality of oligonucleotides which share the same base sequence of the same pattern of chiral linkage phosphorus stereochemistry (e.g., comprising one or more of Rp and/or Sp, wherein each chiral linkage phosphorus is independently Rp or Sp), at least 50%, 60%, 70%, 75%, 80%, 85%, 90%, 95%, or 99% of all oligonucleotides in the composition that share the same base sequence as oligonucleotides of the plurality share the same pattern of chiral linkage phosphorus stereochemistry or are oligonucleotides of the plurality. In some embodiments, in provided compositions comprising a plurality of oligonucleotides which share the same base sequence of the same pattern of chiral linkage phosphorus stereochemistry, at least 50%, 60%, 70%, 75%, 80%, 85%, 90%, 95%, or 99% of all oligonucleotides in the composition that share the same constitution as oligonucleotides of the plurality share the same pattern of chiral linkage phosphorus stereochemistry or are oligonucleotides of the plurality.

In some embodiments, the present disclosure describes useful technologies for assessing oligonucleotide and compositions thereof. For example, various technologies of the present disclosure are useful for assessing adenosine modification. As appreciated by those skilled in the art, in some embodiments, modification/editing of adenosine can be assessed through sequencing, mass spectrometry, assessment (e.g., levels, activities, etc.) of products (e.g., RNA, protein, etc.) of modified nucleic acids (e.g., wherein adenosines of target nucleic acids are converted to inosines), etc., optionally in view of other components (e.g., ADAR proteins) presence in modification systems (e.g., an in vitro system, an ex vivo system, cells, tissues, organs, organisms, subjects, etc.). Those skilled in the art will appreciate that oligonucleotides which provide adenosine modification of a target nucleic acid can also provide modified nucleic acid (e.g., wherein a target adenosine is converted into I) and one or more products thereof (e.g., mRNA, proteins, etc.). Certain useful technologies are described in the Examples.

As described herein, oligonucleotides and compositions of the present disclosure may be provided/utilized in various forms. In some embodiments, the present disclosure provides compositions comprising one or more forms of oligonucleotides, e.g., acid forms (e.g., in which natural phosphate linkages exist as —O(P(O)(OH)—O—, phosphorothioate internucleotidic linkages exist as —O(P(O)(SH)—O—), base forms, salt forms (e.g., in which natural phosphate linkages exist as salt forms (e.g., sodium salt (—O(P(O)(O⁻Na⁺)—O—), phosphorothioate internucleotidic linkages exist as salt forms (e.g., sodium salt (—O(P(O)(S⁻Na⁺)—O—) etc. As appreciated by those skilled in the art, oligonucleotides can exist in various salt forms, including pharmaceutically acceptable salts, and in solutions (e.g., various aqueous buffering system), cations may dissociate from anions. In some embodiments, the present disclosure provides a pharmaceutical composition comprising a provided oligonucleotide and/or one or more pharmaceutically acceptable salts thereof, and a pharmaceutically acceptable carrier. In some embodiments, pharmaceutical compositions are chirally controlled oligonucleotide compositions.

Provided technologies can be utilized for various purposes. For example, those skilled in the art will appreciate that provided technologies are useful for many purposes involving modification of adenosine, e.g., correction of G to A mutations, modulate levels of certain nucleic acids and/or products encoded thereby (e.g., reducing levels of proteins by introducing A to G/I modifications), modulation of splicing, modulation of translation (e.g., modulating translation start and/or stop site by introducing A to G/I modifications), etc.

In some embodiments, the present disclosure provides technologies for preventing or treating a condition, disorder or disease that is amenable to an adenosine modification, e.g. conversion of A to I or G. As appreciated by those skilled in the art, I may perform one or more functions of G, e.g., in base pairing, translation, etc. In some embodiments, a G to A mutation may be corrected through conversion of A to I so that one or more products, e.g., proteins, of the G-version nucleic acid can be produced. In some embodiments, the present disclosure provides technologies for preventing or treating a condition, disorder or disease associated with a mutation, comprising administering to a subject susceptible thereto or suffering therefrom a provided oligonucleotide or composition thereof, which oligonucleotide or composition can edit a mutation. In some embodiments, the present disclosure provides technologies for preventing or treating a condition, disorder or disease associated with a G to A mutation, comprising administering to a subject susceptible thereto or suffering therefrom a provided oligonucleotide or composition thereof, which oligonucleotide or composition can modify an A. In some embodiments, provided technologies modify an A in a transcript, e.g., RNA transcript. In some embodiments, an A is converted into an I. In some embodiments, during translation protein synthesis machineries read I as G. In some embodiments, an A form encodes one or more proteins that have one or more higher desired activities and/or one or more better desired properties compared those encoded by its corresponding G form. In some embodiments, an A form provides higher levels, compared to its corresponding G form, of one or more proteins that have one or more higher desired activities and/or one or more better desired properties. In some embodiments, products encoded by an A form are structurally different (e.g., longer, in some embodiments, full length proteins) from those encoded by its corresponding G form. In some embodiments, an A form provides structurally identical products (e.g., proteins) compared to its corresponding G form.

As those skilled in the art will appreciate, many conditions, disorders or diseases are associated with mutations that can be modified by provided technologies and can be prevented and/or treated using provided technologies. For example, it is reported that there are over 20,000 conditions, disorders or diseases are associated with G to A mutation and can benefit from A to I editing.

### BRIEF DESCRIPTION OF THE DRAWINGS

FIG. 1. Provided technologies with various sugar modification patterns can provide desired activities. (a) ADAR1- and (b) ADAR2-mediated editing. Oligonucleotides all have the same sequence targeting a premature UAG stop codon within the cLuc coding sequence. 293T cells were transfected with ADAR1 and ADAR2, respectively, luciferase reporter construct and indicated compositions. cLuc activity was measure and normalized to Gluc expression in mock treated samples (n=2 biological replicates).

FIG. 2. Provided technologies comprising various internucleotidic linkage modifications can provide desired activities. (a) and (b): Compositions all have the same sequence targeting a premature UAG stop codon within the cLuc coding sequence. 293T cells were transfected with ADAR1 or ADAR2, luciferase reporter construct and indicated compositions. cLuc activity was measure and normalized to Gluc expression in mock treated samples (n=2 biological replicates).

FIG. 3. Provided technologies comprising various sugar modifications can provide desired activities. Compositions all target a premature UAG stop codon within the cLuc coding sequence. 293T cells were transfected with ADAR1 or ADAR2, luciferase reporter construct and indicated compositions. cLuc activity was measure and normalized to Gluc expression in mock treated samples (n=2 biological replicates).

FIG. 4. Provided technologies comprising various sugar types can provide desired activities. Compositions all have the same sequence targeting a premature UAG stop codon within the cLuc coding sequence. 293T cells were transfected with ADAR1 or ADAR2, luciferase reporter construct and indicated compositions. cLuc activity was measure and normalized to Gluc expression in mock treated samples (n=2 biological replicates).

FIG. 5. Provided technologies can provide desired activities with short sequences. Compositions all target a premature UAG stop codon within the cLuc coding sequence. 293T cells were transfected with ADAR1 (a and b) or ADAR2 (c and d), luciferase reporter construct and indicated compositions. cLuc activity was measured and normalized to Gluc expression in mock treated samples (n=2 biological replicates).

FIG. 6. Provided technologies can provide desired activities in various cell types without exogenous ADAR. FIG. 6 depicts editing of an endogenous target (TAG site in 3′ UTR of actin) without exogenous ADAR in different cell types. Cell were transfected with 50 nM oligonucleotides and editing was measured 48 hours later. (N=1 for RPE and NHBE cells, N=2 biological replicates for Hepatocytes)

FIG. 7. Provided technologies comprising various numbers of mismatches can provide desired activities. Compositions all target a premature UAG stop codon within the cLuc coding sequence and have 0-2 mismatches. 293T cells were transfected with ADAR1 or ADAR2, luciferase reporter construct and indicated compositions. cLuc activity was measured at 48 and 96 hrs and normalized to Gluc expression in mock treated samples (n=2 biological replicates).

FIG. 8. Provided technologies comprising various patterns of mismatches can provide desired activities. Compositions all target a premature UAG stop codon within the cLuc coding sequence. 293T cells were transfected with ADAR1 or ADAR2, luciferase reporter construct and indicated compositions. cLuc activity was normalized to Gluc expression in mock treated samples (n=2 biological replicates).

FIG. 9. Provided technologies comprising various patterns of mismatches can provide desired activities. Compositions all target a premature UAG stop codon within the cLuc coding sequence. 293T cells were transfected with ADAR1 or ADAR2, luciferase reporter construct and indicated compositions. cLuc activity was normalized to Gluc expression in mock treated samples (n=2 biological replicates).

FIG. 10. Chirally controlled oligonucleotide compositions can provide desired activities. Compositions all target a premature UAG stop codon within the cLuc coding sequence. 293T cells were transfected with ADAR1 or ADAR2, luciferase reporter construct and indicated compositions. cLuc activity was normalized to Gluc expression in mock treated samples (n=2 biological replicates).

FIG. 11. Chirally controlled oligonucleotide compositions can provide desired activities. Compositions all target a premature UAG stop codon within the cLuc coding sequence. 293T cells were transfected with ADAR1 or ADAR2, luciferase reporter construct and indicated compositions at varying oligonucleotide concentrations. cLuc activity was normalized to Gluc expression in mock treated samples (n=2 biological replicates).

FIG. 12. Chirally controlled oligonucleotide compositions can provide significantly higher activities in various cell types without exogenous ADAR. Compositions all target a UAG motif in the 3′UTR of Actin. Cells were treated gymnotically with oligonucleotides at 10 uM dose, or transfected at 50 nM dose. RNA was harvested 48 hours later and percentage of edited transcripts was quantified by Sanger sequencing (n=2 biological replicates).

FIG. 13. Provided technologies can provide desired activities with short sequences without exogenous ADAR. FIG. 13 depicts editing in primary human retinal pigmented epithelial (RPE cells). Compositions all target a UAG motif in the 3′UTR of actin. Primary human RPE cells were transfected with 50 nM of oligonucleotides. RNA was harvested 48 hours later and percentage of edited transcripts was quantified by Sanger sequencing (n=2 biological replicates).

FIG. 14. Chirally controlled oligonucleotide compositions can provide high activities in various cell types without exogenous ADAR. Compositions all target a UAG motif in the 3′UTR of actin. Primary human bronchial epithelial cells were treated gymnotically with 10 uM of oligonucleotides, while primary RPE cells were transfected with 50 nm of oligonucleotides. RNA was harvested 48 hours later and percentage of edited transcripts was quantified by Sanger sequencing (n=2 biological replicates).

FIG. 15. Provided technologies comprising various internucleotidic linkage patterns can provide desired activities. Compositions all have the same base sequence and target a premature UAG stop codon within the cLuc coding sequence. 293T cells were transfected with ADAR1 or ADAR2, luciferase reporter construct and indicated compositions. cLuc activity was normalized to Gluc expression in mock treated samples (n=2 biological replicates).

FIG. 16. Provided technologies comprising various internucleotidic linkage patterns can provide desired activities without exogenous ADAR. Compositions all target a UAG motif in the 3′UTR of Actin. Primary human hepatocytes were treated gymnotically with 3.3 uM oligonucleotides. RNA was harvested 48 hours later and percentage of edited transcripts was quantified by Sanger sequencing (n=2 biological replicates).

FIG. 17. Provided technologies comprising various modifications and chiral control can provide desired activities without exogenous ADAR. Compositions all target a UAG motif in the 3′UTR of Actin. Primary human hepatocytes were transfected with 50 nM of oligonucleotides. RNA was harvested 48 hours later and percentage of edited transcripts was quantified by Sanger sequencing (n=2 biological replicates).

FIG. 18. Provided technologies comprising additional moieties can provide high activities without exogenous ADAR. (a) and (b): Compositions all target an adenosine in the 3′UTR of beta-actin mRNA. Primary human hepatocytes were gymnotically treated at varying concentrations. Editing of target was measured by Sanger sequencing (n=2 biological replicates).

FIG. 19. Provided technologies can provide desired activities without exogenous ADAR. FIG. 19 depicts editing of SERPINA1 (PiZ allele) in primary mouse hepatocytes. Compositions all target an adenosine in the mutant human SERPINA1 transcript (PiZZ allele). Primary hepatocytes (extracted from a mouse model expressing the mutant human transcript) were transfected with 50 nM of oligonucleotides. Editing of target was measured by Sanger sequencing (n=2 biological replicates).

FIG. 20. Provided technologies comprising modified bases can provide high activities without exogenous ADAR. FIG. 20 depicts editing of SERPINA1 (PiZ allele) in primary mouse hepatocytes. Compositions all target an adenosine in the mutant human SERPINA1 transcript (PiZZ allele). Primary hepatocytes (extracted from a mouse model expressing the mutant human transcript) were transfected treated with 50 nM oligonucleotides. Editing of target was measured by Sanger sequencing (n=2 biological replicates). As shown, provided designs comprising modified nucleobases can greatly improve activities.

FIG. 21. Provided technologies comprising modified bases can provide desired activities. Compositions all target a premature UAG stop codon within the cLuc coding sequence. 293T cells were transfected with ADAR1 or ADAR2, luciferase reporter construct and indicated compositions at varying oligonucleotide concentrations. cLuc activity was normalized to Gluc expression in mock treated samples (n=2 biological replicates).

FIG. 22. FIG. 22 depicts an example of oligonucleotide configuration.

FIG. 23. Provided technologies comprising modified bases can provide high activities. Compositions all target a premature UAG stop codon within the cLuc coding sequence. 293T cells were transfected with ADAR1 or ADAR2, luciferase reporter construct and indicated compositions. cLuc activity was normalized to Gluc expression in mock treated samples (n=2 biological replicates).

FIG. 24. Provided technologies comprising chirally controlled oligonucleotide compositions can provide high activities compared to stereorandom oligonucleotide compositions. Compositions target UAG motifs within indicated transcripts using endogenous ADAR. Primary human hepatocytes were transfected with compositions at 50 nM oligonucleotide concentration. Percent editing of targeted transcripts was determined through Sanger sequencing of harvested RNA after 48 hour treatment (n=2 biological replicates). As demonstrated, provided technologies can provide high editing efficiency for various target transcripts.

FIG. 25. Provided technologies comprising oligonucleotides with modified internucleotidic linkages can provide high activities. In some embodiments, provided oligonucleotides comprise phosphorothioate linkages and non-negatively charged internucleotidic linkages such as n001. Compositions target a premature UAG stop codon within the cLuc coding sequence. 293T cells were transfected with ADAR1-p150, luciferase reporter construct, and indicated compositions at 3.3 nM oligonucleotide concentrations. cLuc activity was normalized to Gluc expression in mock treated samples (n=2 biological replicates).

FIG. 26. Provided technologies comprising oligonucleotides comprising additional chemical moieties can provide high activities. Compositions target an adenosine in the 3′UTR of beta-actin mRNA using endogenous ADAR. Primary monkey hepatocytes were gymnotically treated with indicated compositions at indicated concentrations. Editing of target was measured by Sanger sequencing (n=2 biological replicates).

FIG. 27. Provided technologies comprising oligonucleotides with modified internucleotidic linkages, sugar modifications, and/or additional chemical moieties can provide high activities. Compositions target an adenosine in the 3′UTR of beta-actin mRNA using endogenous ADAR. Primary human hepatocytes were gymnotically treated with indicated compositions at indicated oligonucleotide concentrations. Percent editing of target transcripts was determined through Sanger sequencing (n=2 biological replicates).

FIG. 28. Provided technologies comprising oligonucleotides comprising various modified internucleotidic linkages, sugar modifications and/or additional moieties can provide high activities. Compositions target an adenosine in the 3′UTR of beta-actin mRNA using endogenous ADAR. Primary human hepatocytes were gymnotically treated with indicated compositions at indicated oligonucleotide concentrations. Percent editing of target transcripts was determined through Sanger sequencing (n=2 biological replicates). In some embodiments, certain structural elements, e.g., 2′-F modified sugars in second subdomains, Rp phosphorothioate linkages bonded to second subdomain nucleosides, positioning and/or presence or absence of mismatches, and/or non-negatively charged internucleotidic linkages such as n001 at certain locations can improve editing efficiency.

FIG. 29. Provided technologies comprising oligonucleotides with modified internucleotidic linkages, sugar modifications, and/or additional chemical moieties can provide high activities. Primary human hepatocytes were treated gymnotically with indicated oligonucleotide compositions at indicated concentrations. ADAR was endogenous. Compositions target an adenosine in the 3′ UTR of beta-actin mRNA. Percentage of edited transcripts was quantified by Sanger sequencing (n=2 biological replicates).

FIG. 30. Provided technologies comprising oligonucleotides with modified internucleotidic linkages, sugar modifications, and/or additional chemical moieties can provide high activities. Primary human (a; *: not determined) or monkey (b) hepatocytes were treated gymnotically with indicated oligonucleotide compositions at indicated concentrations. ADAR was endogenous. Compositions target an adenosine in the 3′ UTR of beta-actin mRNA. Percentage of edited transcripts was quantified by Sanger sequencing (n=2 biological replicates).

FIG. 31. Chiral control can improve editing efficiency. Compositions all target a premature UAG stop codon within the cLuc coding sequence. 293T cells were transfected with plasmids encoding ADAR1-p110 or -p150, luciferase reporter construct and indicated oligonucleotide compositions. cLuc activity was normalized to Gluc expression in mock treated samples (n=2 biological replicates). Among other things, increased numbers/levels of chirally controlled internucleotidic linkages (e.g., Sp phosphorothioate internucleotidic linkages) improved editing efficiency for both ADAR1-p110 and ADAR1-p150.

FIG. 32. Chiral control can improve editing efficiency. Compositions all target a premature UAG stop codon within the cLuc coding sequence. 293T cells were transfected with plasmids encoding ADAR1-p110 or -p150, luciferase reporter construct and indicated oligonucleotide compositions. cLuc activity was normalized to Gluc expression in mock treated samples (n=2 biological replicates). Among other things, increased numbers/levels of chirally controlled internucleotidic linkages (e.g., Sp phosphorothioate internucleotidic linkages) improved editing efficiency for both ADAR1-p110 and ADAR1-p150.

FIG. 33. Chiral control and modified internucleotidic linkages can improve editing efficiency. (a) Compositions all target a premature UAG stop codon within the cLuc coding sequence. 293T cells were transfected with plasmids encoding ADAR1-p110, luciferase reporter construct and indicated compositions. cLuc activity was normalized to Gluc expression in mock treated samples (n=2 biological replicates). (b) Compositions all target a premature UAG stop codon within the cLuc coding sequence. 293T cells were transfected with plasmids encoding ADAR1-p150, luciferase reporter construct and indicated compositions. cLuc activity was normalized to Gluc expression in mock treated samples (n=2 biological replicates).

FIG. 34. Assessment of oligonucleotides comprising natural phosphate linkages. In some embodiments, natural phosphate linkages can be utilized in accordance with the present disclosure (e.g., numbers, levels, positions, in combination with other structural features (e.g., modifications, patterns, etc.), etc.) to provide oligonucleotide compositions of certain levels of activities. In FIG. 34, natural phosphate linkages can be utilized in oligonucleotides in accordance with the present disclosure. Compositions all target a premature UAG stop codon within the cLuc coding sequence. 293T cells were transfected with plasmids encoding ADAR1-p150 or ADAR2, luciferase reporter construct and indicated compositions. cLuc activity was normalized to Gluc expression in mock treated samples (n=2 biological replicates).

FIG. 35. Various sugar modifications may be utilized to provide oligonucleotide compositions with desired activities. Compositions all target a premature UAG stop codon within the cLuc coding sequence. 293T cells were transfected with plasmids encoding ADAR1-p150 or ADAR2, luciferase reporter construct and indicated compositions. cLuc activity was normalized to Gluc expression in mock treated samples (n=2 biological replicates).

FIG. 36. Various sugar modifications may be utilized to provide oligonucleotide compositions with desired activities. (a) Compositions all target a premature UAG stop codon within the cLuc coding sequence. 293T cells were transfected with plasmids encoding ADAR1-p150 or ADAR2, luciferase reporter construct and indicated compositions. cLuc activity was normalized to Gluc expression in mock treated samples (n=2 biological replicates). (b) Compositions all target a premature UAG stop codon within the cLuc coding sequence. 293T cells were transfected with plasmids encoding ADAR1-p150 or ADAR2, luciferase reporter construct and indicated compositions. cLuc activity was normalized to Gluc expression in mock treated samples (n=2 biological replicates).

FIG. 37. Provided technologies can provide effective editing in primates. Non-human primates (NHP) were dosed with several compositions (WV-37314, WV-37315, and WV-37330). Compositions all target an adenosine in the 3′UTR of beta-actin mRNA. Animals were dosed subcutaneously once a day for 5 consecutive days (5 mg/kg) with indicated compositions (n=2 animals per composition). Liver biopsy samples were collected two days post last dose. Editing of target was measured by Sanger sequencing. All three compositions administered provided significant levels of in vivo editing of ACTB mRNA (25-50% editing) without administration of exogenous ADAR.

FIG. 38. Provided technologies can provide long-lasting editing activities in vivo. Provided compositions were assessed in non-human primates (NHP). Certain data for liver (a) and kidney (b) are illustrated. Compositions all target an adenosine in the 3′UTR of beta-actin mRNA. Animals were dosed subcutaneously once a day for 5 consecutive days (5 mg/kg) with indicated compositions (n=2 animals per oligonucleotide). Liver biopsy samples were collected 2 days post last dose and 45 days post last dose and kidney biopsy samples were collected at 45 days post last dose. Editing of target was measured by Sanger sequencing. Oligonucleotides in tissues were measured by hybridization ELISA.

FIG. 39. Provided technologies can provide effective editing in various systems including neuronal cells. In some embodiments, compositions were assessed in human iCell neurons and iCell astrocytes (a) and (b), respectively). Compositions all target a UAG motif in the 3′UTR of ACTB and comprise oligonucleotides with the same base sequence. Cells were gymnotically treated with indicated compositions at indicated concentrations and RNA was harvested 6 and 5 days later, respectively. Editing of target was measured by Sanger sequencing (n=2-3 biological replicates). Certain initial results for certain additional targets are presented in (c) and (d). Compositions were assessed in human iCell neurons and iCell astrocytes for editing of 6 different target sites. Each composition targets a UAG motif in the indicated transcript. Cells were gymnotically treated with indicated composition at indicated concentrations, and RNA was harvested 6 days later. Editing of target was measured by Sanger sequencing (n=2 biological replicates).

FIG. 40. Mice engineered to express human ADAR1 can provide editing activity profiles more similar to human cells compared to mice not so engineered. Compositions were assessed in primary hepatocytes harvested from a human ADAR1-transgenic mouse, wild-type mouse and human primary hepatocytes. Oligonucleotides administered comprise GalNAc moieties. Certain data for editing of two different transcripts, UGP2 (a) and EEF1A1 (b), are shown. For each target, certain data from three compositions of oligonucleotides with identical base sequence but different modifications were presented. Editing of target was measured by Sanger sequencing (n=2-3 biological replicates). As confirmed, in some embodiments chiral control and/or various modifications can be utilized to effectively improve editing levels in accordance with the present disclosure. For each type of cells, from left to right: (a): WV-38701, WV-38700, and WV-38702; (b): WV-38698, WV-38697, and WV-38699.

FIG. 41. Provided technologies can provide editing in vivo. Certain in vivo data, e.g., from livers of human-ADAR1-transgenic mice were presented. Animals were treated with compositions of oligonucleotides comprising GalNAc. Wild-type (WT) mice were included as controls. Certain data for editing of UAG motifs on two different transcripts, UGP2 (a) and EEF1A1 (b), are shown. For each target, certain data from two compositions of oligonucleotides with identical sequence but different modifications were presented. Three animals in each treatment group were dosed with PBS or 10 mg/kg of indicated compositions on days 1, 3, and 5, and liver biopsies were collected on day 8. (n=3 mice per group). As confirmed, in some embodiments chirally controlled oligonucleotide compositions of oligonucleotides comprising non-negatively charged internucleotidic linkages (e.g., n001) can be utilized to effectively improve editing levels, including in vivo, in accordance with the present disclosure.

FIG. 42. Provided technologies can provide editing in vivo in various tissues including in central nervous system. Compositions were assessed in CNS tissues of human-ADAR1-transgenic mice. Certain data for editing of UGP2 (a) and SRSF1 (b) transcripts were presented. Animals were treated with compositions by ICV injection. Five mice in each group were injected with PBS, 2×50 ug doses of oligonucleotide composition on day 0 and 2, or a single 100 ug dose on day 0. Animals were necropsied on day 7. RNA from indicated tissues was harvested, and editing measured by Sanger sequencing (n=5 mice per group). Oligonucleotide distribution in different brain tissue was also measured by hybridization ELISA. Among other things, it was confirmed that provided oligonucleotides can be delivered to various tissues and provide editing activities therein.

FIG. 43. Reduction of certain proteins using siRNA. Shown are ADAR1 p150 (top), ADAR1 p110 (middle) and vinculin loading control (below) in ARPE-19 cells treated with indicated siRNA reagents with or without IFN-a.

FIG. 44. Certain editing data of endogenous ACTB observed with WV-23928 or WV-27395 with siRNA-mediated depletion of the indicated ADAR, with and without IFN-a treatment. N≥3, mean±SEM. **** P<0.0001 by Welch's two-way ANOVA followed by two-tailed post-hoc test. nd, not detected; NTC non-targeting control.

FIG. 45. FIG. 45. Provided technologies can provide highly specific editing. (a) Scatter plot (top) of variants detected in WV-30298 samples. On-target ACTB editing and off-target edits have >3 LOD score and >5% editing. LOD score calculated by Mutect2 indicates the likelihood odds ratio that a variant exists in treated samples compared with mock samples. Genes with the highest percentage editing and highest LOD scores are labeled. Total RNA coverage (bottom) across replicates for all variants (potential edit sites). (b) Scatter plot (top) of variants and total RNA coverage (bottom) in WV-27458 samples.

FIG. 46. Provided technologies can provide multiplex editing. Presented are certain data in primary human hepatocytes. (a) Percentage editing observed on indicated transcripts in the presence of 20 nM each of a single (Isolated) or multiple (Multiplex) oligonucleotide compositions after transfection of primary human hepatocytes. (b) Percentage editing detected on indicated transcripts in the presence of 1.1 uM each of a single (Isolated) or multiple (Multiplex) GalNAc-conjugated oligonucleotides. N=3, mean±SEM. * P<0.05, *** P<0.001 by two-tailed Welch's t-test. For each target, from left to right: Isolated, Multiplex.

FIG. 47. Mice engineered to express human ADAR1 can provide editing activity profiles more similar to human cells compared to mice not so engineered. Compositions were assessed in primary hepatocytes harvested from a human ADAR1-transgenic mouse, wild-type mouse and human primary hepatocytes. Oligonucleotides administered comprise GalNAc moieties. Certain data for editing of two different transcripts, UGP2 (a-c) and EEF1A1 (d-f), are shown. For each target, certain data from three compositions of oligonucleotides with identical base sequence but different modifications were presented. Editing of target was measured by Sanger sequencing (n=2-3 biological replicates). As confirmed, in some embodiments chiral control and/or various modifications can be utilized to effectively improve editing levels in accordance with the present disclosure.

FIG. 48. Provided technologies provide editing in various cell types including CD8+ T cells. FIG. 48 depicts editing in primary human CD8+ T cells by gymnotic uptake. All oligonucleotides have the same sequence and all target a UAG motif in the 3′UTR of ACTB. Primary T cells were pre-stimulated for 24 or 96 hrs as indicated and then were gymnotically treated with oligonucleotide compositions at indicated concentrations, and RNA was harvested 4 days later. Editing of target was measured by Sanger sequencing (n=2 biological replicates).

FIG. 49. Provided technologies provide editing in various cell types including primary human fibroblasts. FIG. 49 depicts certain editing in primary human fibroblasts by gymnotic uptake and transfection. The oligonucleotide composition WV-37318 targets a UAG motif in the 3′UTR of ACTB. Three different primary human fibroblast lines were treated by transfection (50 nM) or by gymnotic uptake (10 uM) as indicated, and RNA was harvested 60 hours later. Editing of target was measured by Sanger sequencing (n=2 biological replicates).

FIG. 50. Provided technologies provide editing in various cell types including in ex vivo retinal tissue isolated from non-human primate eyes. FIG. 50 depicts certain editing in ex vivo retinal tissue isolated from non-human primate eyes. The oligonucleotide composition targets a UAG motif in the 3′UTR of ACTB. In two independent experiments, eyeballs from NHPs were freshly dissected and retinal tissue was treated with oligonucleotide composition by gymnotic uptake. RNA was harvested 48 hours later. Editing of target was measured by Sanger sequencing (n=4-5 biological replicates per experimental condition).

FIG. 51. Provided technologies comprising various modifications can provide editing. FIG. 51 ((a)-(d)) depicts certain editing in primary human hepatocytes. The oligonucleotide compositions target specific adenosine residues (surrogate site #1, 2, 3, or 4) in a coding sequence of a wild-type SERPINA1 (SA1) transcript. As confirmed, oligonucleotides comprising various modifications, sequences and/or additional chemical moieties can provide desired editing. Primary human hepatocytes were treated with indicated compositions and concentrations. RNA was harvested 48 hours later. Editing of target was measured by Sanger sequencing (n=2 biological replicates).

FIG. 52. Provided technologies comprising various modifications can provide editing. FIG. 52 depicts certain editing in primary human hepatocytes. The oligonucleotide compositions target specific adenosine residues (surrogate site #1, 2, 3, or 4) in a coding sequence of a wild-type SERPINA1 (SA1) transcript. Primary human hepatocytes were treated with indicated oligonucleotide compositions and concentrations. RNA was harvested 48 hours later. Editing of target was measured by Sanger sequencing (n=2 biological replicates).

FIG. 53. Provided technologies comprising various modifications can provide editing. FIG. 53, (a), depicts editing in primary human hepatocytes. The oligonucleotide compositions target a specific adenosine residue (surrogate site #1) in a coding sequence of a wild-type SERPINA1 (SA1) transcript. Primary human hepatocytes were treated with indicated oligonucleotide compositions and concentrations. RNA was harvested 48 hours later. Editing of target was measured by Sanger sequencing (n=2 biological replicates). FIG. 53, (b) depicts editing in primary human hepatocytes. The oligonucleotide compositions target a specific adenosine residue (surrogate site #2) in a coding sequence of a wild-type SERPINA1 (SA1) transcript. Primary human hepatocytes were treated with indicated oligonucleotide compositions and concentrations. RNA was harvested 48 hours later. Editing of target was measured by Sanger sequencing (n=2 biological replicates). FIG. 53, (c), depicts editing in primary human hepatocytes. The oligonucleotide compositions target a specific adenosine residue (surrogate site #1 or #2) in a coding sequence of a wild-type SERPINA1 (SA1) transcript. Primary human hepatocytes were treated with indicated oligonucleotide compositions and concentrations. RNA was harvested 48 hours later. Editing of target was measured by Sanger sequencing (n=2 biological replicates).

FIG. 54. Removing wobbles and/or mismatches may improve editing levels. FIG. 54 depicts editing in primary human and NHP hepatocytes. The oligonucleotide compositions target a specific adenosine residue (surrogate site #1 or #2) in a coding sequence of a WT SERPINA1 (SA1) transcript. Primary human and NHP hepatocytes were treated with indicated oligonucleotide compositions and concentrations. RNA was harvested 48 hours later. Editing of target was measured by Sanger sequencing (n=2 biological replicates).

FIG. 55. Provide technologies can provide editing in NHP and human cells at various concentrations. FIG. 55 depicts editing in primary human and NHP hepatocytes. The oligonucleotide compositions target a specific adenosine residue (surrogate site #1 or #2) in a coding sequence of a wild-type SERPINA1 (SA1) transcript. Both oligonucleotide compositions have a G-U wobble against the NHP mRNA sequence. Primary human and NHP hepatocytes were treated with indicated oligonucleotide compositions and concentrations. RNA was harvested 48 hours later. Editing of target was measured by Sanger sequencing (n=2 biological replicates).

FIG. 56. Provide technologies comprising various modifications can provide editing. FIG. 56 depicts editing in primary NHP hepatocytes. The oligonucleotide compositions target a specific adenosine residue (surrogate site #2) in a coding sequence of a wild-type SERPINA1 (SA1) transcript. Primary NHP hepatocytes were treated with indicated oligonucleotide compositions and concentrations. RNA was harvested 48 hours later. Editing of target was measured by Sanger sequencing (n=2 biological replicates).

FIG. 57. Provide technologies comprising various modifications including base modifications can provide editing. The oligonucleotide compositions all target a premature UAG stop codon within a cLuc coding sequence. 293T cells were transfected with ADAR-p110 or ADAR1-p150, luciferase reporter construct and indicated oligonucleotide compositions. cLuc activity was measured and normalized to Gluc expression in mock treated samples (n=2 biological replicates).

FIG. 58. Provide technologies comprising various modifications including abasic units can provide editing. The oligonucleotide compositions all target a premature UAG stop codon within a cLuc coding sequence. 293T cells were transfected with ADAR-p110 or ADAR1-p150, luciferase reporter construct and indicated oligonucleotide compositions. cLuc activity was measured and normalized to Gluc expression in mock treated samples (n=2 biological replicates).

FIG. 59. Provide technologies comprising various modifications including base modifications can provide editing. FIG. 59 depicts editing by compositions of oligonucleotides comprising modified nucleobases at positions across from target sites. The oligonucleotide compositions all target a PiZ mutation of a SERPINA1 (SA1) transcript. ARPE cells stably expressing the SA1-PiZ allele from a lentiviral vector were transfected with indicated oligonucleotide compositions. RNA was collected 3 days later. RNA editing was quantified by Sanger sequencing (n=2 biological replicates).

FIG. 60. Provide technologies comprising various types of nucleobases and sugars can provide editing. The oligonucleotide compositions all target a PiZ mutation of the SERPINA1 (SA1) transcript. 293T cells were transfected with a plasmid expressing the SA1-PiZ allele, ADAR1-p110 or ADAR1-p150, and indicated oligonucleotide compositions. RNA was collected 48 hours later. RNA editing was quantified by Sanger sequencing (n=2 biological replicates).

FIG. 61. Provide technologies comprising various types of nucleobases and sugars can provide editing. The oligonucleotide compositions all target a PiZ mutation of a SERPINA1 (SA1) transcript. Freshly collected primary hepatocytes from a SA1-PiZ-mouse model were treated with indicated oligonucleotide compositions. RNA was collected 48 hours later. RNA editing was quantified by Sanger sequencing (n=2 biological replicates).

FIG. 62. Provide technologies comprising various types of nucleobases and sugars can provide editing. The oligonucleotide compositions all target the PiZ mutation of the SERPINA1 (SA1) transcript. ARPE cells stably expressing the SA1-PiZ allele from a lentiviral vector were transfected with indicated oligonucleotide compositions. RNA was collected 3 days later. RNA editing was quantified by Sanger sequencing (n=2 biological replicates).

FIG. 63. Provide technologies comprising inosine can provide editing. The oligonucleotide compositions all target a PiZ mutation of a SERPINA1 (SA1) transcript. ARPE cells stably expressing the SA1-PiZ allele from a lentiviral vector were transfected with indicated oligonucleotide compositions. RNA was collected 3 days later. RNA editing was quantified by Sanger sequencing (n=2 biological replicates).

FIG. 64. Provide technologies comprising various nucleobases at sites opposite to target sites can provide editing. FIG. 64 depicts editing of a premature UAG stop codon within a cLuc coding sequence.

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof (e.g., base sequence, sugar modifications, internucleotidic linkages, linkage phosphorus stereochemistry, patterns thereof, etc.) is from 5′ to 3′. As those skilled in the art will appreciate, in some embodiments, oligonucleotides may be provided and/or utilized as salt forms, particularly pharmaceutically acceptable salt forms, e.g., sodium salts. As those skilled in the art will also appreciate, in some embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H⁺) are of the same constitution and/or structure, such individual oligonucleotides may properly be considered to be of the same constitution and/or structure.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1˜4 carbon atoms (e.g., C1-C4 for straight chain lower alkyls).

Alkynyl: As used herein, the term “alkynyl” refers to an aliphatic group, as defined herein, having one or more triple bonds.

Analog: The term “analog” includes any chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase; etc.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, each monocyclic ring unit is aromatic. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Characteristic portion: As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Chiral control: As used herein, “chiral control” refers to control of the stereochemical designation of the chiral linkage phosphorus in a chiral internucleotidic linkage within an oligonucleotide. As used herein, a chiral internucleotidic linkage is an internucleotidic linkage whose linkage phosphorus is chiral. In some embodiments, a control is achieved through a chiral element that is absent from the sugar and base moieties of an oligonucleotide, for example, in some embodiments, a control is achieved through use of one or more chiral auxiliaries during oligonucleotide preparation, which chiral auxiliaries often are part of chiral phosphoramidites used during oligonucleotide preparation. In contrast to chiral control, a person having ordinary skill in the art will appreciate that conventional oligonucleotide synthesis which does not use chiral auxiliaries cannot control stereochemistry at a chiral internucleotidic linkage if such conventional oligonucleotide synthesis is used to form the chiral internucleotidic linkage. In some embodiments, the stereochemical designation of each chiral linkage phosphorus in each chiral internucleotidic linkage within an oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share a common base sequence, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). In some embodiments, a chirally controlled oligonucleotide composition comprises a plurality of oligonucleotides (or nucleic acids) that share: 1) a common base sequence, 2) a common pattern of backbone linkages, and 3) a common pattern of backbone phosphorus modifications, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). Level of the plurality of oligonucleotides (or nucleic acids) in a chirally controlled oligonucleotide composition is pre-determined/controlled or enriched (e.g., through chirally controlled oligonucleotide preparation to stereoselectively form one or more chiral internucleotidic linkages) compared to a random level in a non-chirally controlled oligonucleotide composition. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, a level is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications, or of all oligonucleotides in a composition that share a common base sequence, a common patter of base modifications, a common pattern of sugar modifications, a common pattern of internucleotidic linkage types, and/or a common pattern of internucleotidic linkage modifications. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 1%-100% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In some embodiments, oligonucleotides (or nucleic acids) of a plurality share the same pattern of sugar and/or nucleobase modifications, in any. In some embodiments, oligonucleotides (or nucleic acids) of a plurality are various forms of the same oligonucleotide (e.g., acid and/or various salts of the same oligonucleotide). In some embodiments, oligonucleotides (or nucleic acids) of a plurality are of the same constitution. In some embodiments, level of the oligonucleotides (or nucleic acids) of the plurality is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides (or nucleic acids) in a composition that share the same constitution as the oligonucleotides (or nucleic acids) of the plurality. In some embodiments, each chiral internucleotidic linkage is a chiral controlled internucleotidic linkage, and the composition is a completely chirally controlled oligonucleotide composition. In some embodiments, oligonucleotides (or nucleic acids) of a plurality are structurally identical. In some embodiments, a chirally controlled internucleotidic linkage has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In some embodiments, a chirally controlled internucleotidic linkage has a diastereopurity of at least 95%. In some embodiments, a chirally controlled internucleotidic linkage has a diastereopurity of at least 96%. In some embodiments, a chirally controlled internucleotidic linkage has a diastereopurity of at least 97%. In some embodiments, a chirally controlled internucleotidic linkage has a diastereopurity of at least 98%. In some embodiments, a chirally controlled internucleotidic linkage has a diastereopurity of at least 99%. In some embodiments, a percentage of a level is or is at least (DS)^{nc}, wherein DS is a diastereopurity as described in the present disclosure (e.g., 90%, 91%, 92%, 93%, 94%, 95%, 96%, 97%, 98%, 99% or 99.5% or more) and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, a percentage of a level is or is at least (DS)^{nc}, wherein DS is 95%-100%. For example, when DS is 99% and nc is 10, the percentage is or is at least 90% ((99%)¹⁰ 0.90=90%). In some embodiments, level of a plurality of oligonucleotides in a composition is represented as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy). In some embodiments, not all chiral internucleotidic linkages are chiral controlled internucleotidic linkages, and the composition is a partially chirally controlled oligonucleotide composition. In some embodiments, a non-chirally controlled internucleotidic linkage has a diastereopurity of less than about 80%, 75%, 70%, 65%, 60%, 55%, or of about 50%, as typically observed in stereorandom oligonucleotide compositions (e.g., as appreciated by those skilled in the art, from traditional oligonucleotide synthesis, e.g., the phosphoramidite method). In some embodiments, oligonucleotides (or nucleic acids) of a plurality are of the same type. In some embodiments, a chirally controlled oligonucleotide composition comprises non-random or controlled levels of individual oligonucleotide or nucleic acids types. For instance, in some embodiments a chirally controlled oligonucleotide composition comprises one and no more than one oligonucleotide type. In some embodiments, a chirally controlled oligonucleotide composition comprises more than one oligonucleotide type. In some embodiments, a chirally controlled oligonucleotide composition comprises multiple oligonucleotide types. In some embodiments, a chirally controlled oligonucleotide composition is a composition of oligonucleotides of an oligonucleotide type, which composition comprises a non-random or controlled level of a plurality of oligonucleotides of the oligonucleotide type.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, each monocyclic ring unit is aromatic. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Identity: As used herein, the term “identity” refers to the overall relatedness between polymeric molecules, e.g., between nucleic acid molecules (e.g., oligonucleotides, DNA, RNA, etc.) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Internucleotidic linkage: As used herein, the phrase “internucleotidic linkage” refers generally to a linkage linking nucleoside units of an oligonucleotide or a nucleic acid. In some embodiments, an internucleotidic linkage is a phosphodiester linkage, as extensively found in naturally occurring DNA and RNA molecules (natural phosphate linkage (—OP(═O)(OH)O—), which as appreciated by those skilled in the art may exist as a salt form). In some embodiments, an internucleotidic linkage is a modified internucleotidic linkage (not a natural phosphate linkage). In some embodiments, an internucleotidic linkage is a “modified internucleotidic linkage” wherein at least one oxygen atom or —OH of a phosphodiester linkage is replaced by a different organic or inorganic moiety. In some embodiments, such an organic or inorganic moiety is selected from ═S, ═Se, ═NR′, —SR′, —SeR′, —N(R′)₂, B(R′)₃, —S—, —Se—, and —N(R′)—, wherein each R′ is independently as defined and described in the present disclosure. In some embodiments, an internucleotidic linkage is a phosphotriester linkage, phosphorothioate linkage (or phosphorothioate diester linkage, —OP(═O)(SH)O—, which as appreciated by those skilled in the art may exist as a salt form), or phosphorothioate triester linkage. In some embodiments, a modified internucleotidic linkage is a phosphorothioate linkage. In some embodiments, an internucleotidic linkage is one of, e.g., PNA (peptide nucleic acid) or PMO (phosphorodiamidate Morpholino oligomer) linkage. In some embodiments, a modified internucleotidic linkage is a non-negatively charged internucleotidic linkage. In some embodiments, a modified internucleotidic linkage is a neutral internucleotidic linkage (e.g., n001 in certain provided oligonucleotides). It is understood by a person of ordinary skill in the art that an internucleotidic linkage may exist as an anion or cation at a given pH due to the existence of acid or base moieties in the linkage. In some embodiments, a modified internucleotidic linkages is a modified internucleotidic linkages designated as s, s1, s2, s3, s4, s5, s6, s7, s8, s9, s10, s11, s12, s13, s14, s15, s16, s17 and s18 as described in WO 2017/210647.

In vitro: As used herein, the term “in vitro” refers to events that occur in an artificial environment, e.g., in a test tube or reaction vessel, in cell culture, etc., rather than within an organism (e.g., animal, plant and/or microbe).

In vivo: As used herein, the term “in vivo” refers to events that occur within an organism (e.g., animal, plant and/or microbe).

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is chiral (e.g., as in phosphorothioate internucleotidic linkages). In some embodiments, a linkage phosphorus atom is achiral (e.g., as in natural phosphate linkages).

Modified nucleobase: The terms “modified nucleobase”, “modified base” and the like refer to a chemical moiety which is chemically distinct from a nucleobase, but which is capable of performing at least one function of a nucleobase. In some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising an at least complementary sequence of bases.

Modified nucleotide: The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

Modified sugar: The term “modified sugar” refers to a moiety that can replace a sugar. A modified sugar mimics the spatial arrangement, electronic properties, or some other physicochemical property of a sugar. In some embodiments, as described in the present disclosure, a modified sugar is substituted ribose or deoxyribose. In some embodiments, a modified sugar comprises a 2′-modification. Examples of useful 2′-modification are widely utilized in the art and described herein. In some embodiments, a 2′-modification is 2′-F. In some embodiments, a 2′-modification is 2′-OR, wherein R is optionally substituted C₁₋₁₀ aliphatic. In some embodiments, a 2′-modification is 2′-OMe. In some embodiments, a 2′-modification is 2′-MOE. In some embodiments, a modified sugar is a bicyclic sugar (e.g., a sugar used in LNA, BNA, etc.). In some embodiments, in the context of oligonucleotides, a modified sugar is a sugar that is not ribose or deoxyribose as typically found in natural RNA or DNA.

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA) or a combination thereof. These terms refer to the primary structure of the molecules and, thus, include double- and single-stranded DNA, and double- and single-stranded RNA. These terms include, as equivalents, analogs of either RNA or DNA comprising modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotidic linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly—refers to a nucleic acid containing 2 to about 10,000 nucleotide monomer units and wherein the prefix oligo—refers to a nucleic acid containing 2 to about 200 nucleotide monomer units.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, a naturally-occurring nucleobases are modified adenine, guanine, uracil, cytosine, or thymine. In some embodiments, a naturally-occurring nucleobases are methylated adenine, guanine, uracil, cytosine, or thymine. In some embodiments, a nucleobase comprises a heteroaryl ring wherein a ring atom is nitrogen, and when in a nucleoside, the nitrogen is bonded to a sugar moiety. In some embodiments, a nucleobase comprises a heterocyclic ring wherein a ring atom is nitrogen, and when in a nucleoside, the nitrogen is bonded to a sugar moiety. In some embodiments, a nucleobase is a “modified nucleobase,” a nucleobase other than adenine (A), guanine (G), uracil (U), cytosine (C), and thymine (T). In some embodiments, a modified nucleobase is substituted A, T, C, G or U. In some embodiments, a modified nucleobase is a substituted tautomer of A, T, C, G, or U. In some embodiments, a modified nucleobases is methylated adenine, guanine, uracil, cytosine, or thymine. In some embodiments, a modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs. In some embodiments, a nucleobase is optionally substituted A, T, C, G, or U, or an optionally substituted tautomer of A, T, C, G, or U. In some embodiments, a “nucleobase” refers to a nucleobase unit in an oligonucleotide or a nucleic acid (e.g., A, T, C, G or U as in an oligonucleotide or a nucleic acid).

Nucleoside: The term “nucleoside” refers to a moiety wherein a nucleobase or a modified nucleobase is covalently bound to a sugar or a modified sugar. In some embodiments, a nucleoside is a natural nucleoside, e.g., adenosine, deoxyadenosine, guanosine, deoxyguanosine, thymidine, uridine, cytidine, or deoxycytidine. In some embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In some embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In some embodiments, a “nucleoside” refers to a nucleoside unit in an oligonucleotide or a nucleic acid.

Nucleotide: The term “nucleotide” as used herein refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs. In some embodiments, a “nucleotide” refers to a nucleotide unit in an oligonucleotide or a nucleic acid.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides.

Oligonucleotides of the present disclosure can be of various lengths. In particular embodiments, oligonucleotides can range from about 2 to about 200 nucleosides in length. In various related embodiments, oligonucleotides, single-stranded, double-stranded, or triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In some embodiments, the oligonucleotide is from about 9 to about 39 nucleosides in length. In some embodiments, the oligonucleotide is at least 4, 5, 6, 7, 8, 9, 10, 11, 12, 13, 14, 15, 16, 17, 18, 19, 20, 21, 22, 23, 24, or 25 nucleosides in length. In some embodiments, the oligonucleotide is at least 4 nucleosides in length. In some embodiments, the oligonucleotide is at least 5 nucleosides in length. In some embodiments, the oligonucleotide is at least 6 nucleosides in length. In some embodiments, the oligonucleotide is at least 7 nucleosides in length. In some embodiments, the oligonucleotide is at least 8 nucleosides in length. In some embodiments, the oligonucleotide is at least 9 nucleosides in length. In some embodiments, the oligonucleotide is at least 10 nucleosides in length. In some embodiments, the oligonucleotide is at least 11 nucleosides in length. In some embodiments, the oligonucleotide is at least 12 nucleosides in length. In some embodiments, the oligonucleotide is at least 15 nucleosides in length. In some embodiments, the oligonucleotide is at least 15 nucleosides in length. In some embodiments, the oligonucleotide is at least 16 nucleosides in length. In some embodiments, the oligonucleotide is at least 17 nucleosides in length. In some embodiments, the oligonucleotide is at least 18 nucleosides in length. In some embodiments, the oligonucleotide is at least 19 nucleosides in length. In some embodiments, the oligonucleotide is at least 20 nucleosides in length. In some embodiments, the oligonucleotide is at least 25 nucleosides in length. In some embodiments, the oligonucleotide is at least 30 nucleosides in length. In some embodiments, each nucleoside counted in an oligonucleotide length independently comprises a nucleobase comprising a ring having at least one nitrogen ring atom. In some embodiments, each nucleoside counted in an oligonucleotide length independently comprises A, T, C, G, or U, or optionally substituted A, T, C, G, or U, or an optionally substituted tautomer of A, T, C, G or U.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, phosphorothioate triester, etc.), pattern of backbone chiral centers [i.e., pattern of linkage phosphorus stereochemistry (Rp/Sp)], and pattern of backbone phosphorus modifications. In some embodiments, oligonucleotides of a common designated “type” are structurally identical to one another.

One of skill in the art will appreciate that synthetic methods of the present disclosure provide for a degree of control during the synthesis of an oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In some embodiments, an oligonucleotide strand is designed and/or selected in advance to have a particular combination of stereocenters at the linkage phosphorus. In some embodiments, an oligonucleotide strand is designed and/or determined to have a particular combination of modifications at the linkage phosphorus. In some embodiments, an oligonucleotide strand is designed and/or selected to have a particular combination of bases. In some embodiments, an oligonucleotide strand is designed and/or selected to have a particular combination of one or more of the above structural characteristics. In some embodiments, the present disclosure provides compositions comprising or consisting of a plurality of oligonucleotide molecules (e.g., chirally controlled oligonucleotide compositions). In some embodiments, all such molecules are of the same type (i.e., are structurally identical to one another). In some embodiments, however, provided compositions comprise a plurality of oligonucleotides of different types, typically in pre-determined relative amounts.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄ CH(OR^{◯})₂; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃)—(CH₂)₀₋₄N(R^{◯}₂—(CH₂)₀₋₄N(R^{◯})C(O)R^{◯}; —N(R^{◯}C(S)R^{◯}); —(CH₂)₀₋₄N(R^{◯}C(O)NR^{◯}₂); —N(R^{◯}C(S)NR^{◯}₂); —(CH₂)₀₋₄N(R^{◯}C(O)OR^{◯});)—N(R^{◯}N(R^{◯}C(O)R^{◯}); —N(R^{◯}N(R^{◯}C(O)NR^{◯})₂);—N(R^{◯}N(R^{◯}C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄ SR^{◯}, —SC(S)SR^{◯}; —(CH₂)₀₋₄ SC(O)R^{◯}; —(CH₂)₀₋₄C(O)NR^{◯}₂; —C(S)NR^{◯}₂; —C(S)SR^{◯}; —(CH₂)₀₋₄OC(O)NR^{◯}₂); —C(O)N(OR^{◯}R^{◯}; —C(O)C(O)R^{◯}; —C(O)CH₂C(O)R^{◯}); —C(NOR^{◯}R^{◯}; —(CH₂)₀₋₄ SSR^{◯}; —(CH₂)₀₋₄S(O)₂R^{◯}; —(CH₂)₀₋₄ S(O)₂OR^{◯}; —(CH₂)₀₋₄OS(O)₂R^{◯}; —S(O)₂NR^{◯}₂; —(CH₂)₀₋₄S(O)R^{◯}); —N(R^{◯}S(O)₂NR^{◯}₂; N(R^{◯}S(O)₂NR^{◯}₂); —N(R^{◯}S(O)₂R^{◯})—N(OR^{◯}R^{◯}; —C(NH)NR^{◯}₂); —Si(R^{◯}₃); —OSi(R^{◯})₃; —B(R^{◯})₂; —OB(R^{◯})₂; —OB(OR^{◯})₂; —P(R^{◯})₂; —P(OR^{◯})₂; —P(R^{◯})(OR^{◯}); —OP(R^{◯})₂; —OP(OR^{◯})₂; —OP(R^{◯})(OR^{◯}); —P(O)(R^{◯})₂; —P(O)(OR^{◯})₂; —OP(O)(R^{◯})₂; —OP(O)(OR^{◯})₂; —OP(O)(OR^{◯})(SR^{◯}); —SP(O)(R^{◯})₂; —SP(O)(OR^{◯})₂; —N(R^{◯})P(O)(R^{◯})₂; —N(R^{◯})P(O)(OR^{◯})₂; —P(R^{◯})₂[B(R^{◯})₃]; —P(OR^{◯})₂[B(R^{◯})₃]; —OP(R^{◯})₂[B(R^{◯})₃]; —OP(OR^{◯}₂[B(R^{◯})₃]; —(C₁₋₄ straight or branched) alkylene)O—N(R^{◯}₂; or —(C₁₋₄ straight or branched)alkylene)C(O)O—N(R^{◯}₂, wherein each R^{◯} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{◯}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{◯}(or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂S—R^{●}, —(CH₂)₀₋₂SH, —(CH₂)₀₋₂NH₂, —(CH₂)₀₋₂NHR^{●}, —(CH₂)₀₋₂NH₂, —NO₂, —SiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{◯} include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R^{*}₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each IV is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

P-modification: as used herein, the term “P-modification” refers to any modification at the linkage phosphorus other than a stereochemical modification. In some embodiments, a P-modification comprises addition, substitution, or removal of a pendant moiety covalently attached to a linkage phosphorus.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations. In some embodiments, each phosphorothioate and phosphate group independently exists in its salt form (e.g., if sodium salt, —O—P(O)(SNa)—O— and —O—P(O)(ONa)—O—, respectively). In some embodiments, each phosphorothioate and phosphate internucleotidic linkage independently exists in its salt form (e.g., if sodium salt, —O—P(O)(SNa)—O— and —O—P(O)(ONa)—O—, respectively). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Predetermined: By predetermined (or pre-determined) is meant deliberately selected or non-random or controlled, for example as opposed to randomly occurring, random, or achieved without control. Those of ordinary skill in the art, reading the present specification, will appreciate that the present disclosure provides technologies that permit selection of particular chemistry and/or stereochemistry features to be incorporated into oligonucleotide compositions, and further permits controlled preparation of oligonucleotide compositions having such chemistry and/or stereochemistry features. Such provided compositions are “predetermined” as described herein. Compositions that may contain certain oligonucleotides because they happen to have been generated through a process that are not controlled to intentionally generate the particular chemistry and/or stereochemistry features are not “predetermined” compositions. In some embodiments, a predetermined composition is one that can be intentionally reproduced (e.g., through repetition of a controlled process). In some embodiments, a predetermined level of a plurality of oligonucleotides in a composition means that the absolute amount, and/or the relative amount (ratio, percentage, etc.) of the plurality of oligonucleotides in the composition is controlled. In some embodiments, a predetermined level of a plurality of oligonucleotides in a composition is achieved through chirally controlled oligonucleotide preparation.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. June 2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” or “test subject” refers to any organism to which a compound (e.g., an oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially identical or complementary to a second sequence is not fully identical or complementary to the second sequence, but is mostly or nearly identical or complementary to the second sequence. In some embodiments, an oligonucleotide with a substantially complementary sequence to another oligonucleotide or nucleic acid forms duplex with the oligonucleotide or nucleic acid in a similar fashion as an oligonucleotide with a fully complementary sequence. In addition, one of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars. In some embodiments, a sugar is a RNA or DNA sugar (ribose or deoxyribose). In some embodiments, a sugar is a modified ribose or deoxyribose sugar, e.g., 2′-modified, 5′-modified, etc. As described herein, in some embodiments, when used in oligonucleotides and/or nucleic acids, modified sugars may provide one or more desired properties, activities, etc. In some embodiments, a sugar is optionally substituted ribose or deoxyribose. In some embodiments, a “sugar” refers to a sugar unit in an oligonucleotide or a nucleic acid.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

As those skilled in the art will appreciate, methods and compositions described herein relating to provided compounds (e.g., oligonucleotides) generally also apply to pharmaceutically acceptable salts of such compounds.

### DESCRIPTION OF CERTAIN EMBODIMENTS

Oligonucleotides are useful in various therapeutic, diagnostic, and research applications. Use of naturally occurring nucleic acids is limited, for example, by their susceptibility to endo- and exo-nucleases. As such, various synthetic counterparts have been developed to circumvent these shortcomings and/or to further improve various properties and activities. These include synthetic oligonucleotides that contain chemical modifications, e.g., base modifications, sugar modifications, backbone modifications, etc., which, among other things, render these molecules less susceptible to degradation and improve other properties and/or activities.

From a structural point of view, modifications to internucleotidic linkages can introduce chirality, and certain properties and activities may be affected by configurations of linkage phosphorus atoms of oligonucleotides. For example, binding affinity, sequence specific binding to complementary RNA, stability to nucleases, activities, delivery, pharmacokinetics, etc. can be affected by, inter alia, chirality of backbone linkage phosphorus atoms.

Among other things, the present disclosure utilizes technologies for controlling various structural elements, e.g., sugar modifications and patterns thereof, nucleobase modifications and patterns thereof, modified internucleotidic linkages and patterns thereof, linkage phosphorus stereochemistry and patterns thereof, additional chemical moieties (moieties that are not typically in an oligonucleotide chain) and patterns thereof, etc. With the capability to fully control structural elements of oligonucleotides, the present disclosure provides oligonucleotides with improved and/or new properties and/or activities for various applications, e.g., as therapeutic agents, probes, etc. For example, as demonstrated herein, provided oligonucleotides and compositions thereof are particularly powerful for editing target adenosine in target nucleic acids to, in some embodiments, correct a G to A mutation by converting A to I.

In some embodiments, an oligonucleotide comprises a sequence that is identical to or is completely or substantially complementary to 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, typically 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, contiguous bases of a nucleic acid (e.g., DNA, pre-mRNA, mRNA, etc.). In some embodiments, a nucleic acid is a target nucleic acid comprising one or more target adenosine. In some embodiments, a target nucleic acid comprises one and no more than one target adenosine. In some embodiments, an oligonucleotide can hybridize with a target nucleic acid. In some embodiments, such hybridization facilitates modification of A (e.g., conversion of A to I) by, e.g., ADAR1, ADAR2, etc., in a nucleic acid or a product thereof.

In some embodiments, the present disclosure provides an oligonucleotide, wherein the oligonucleotide has a base sequence which is, or comprises about 10-40, about 15-40, about 20-40, or at least 27, at least 28, at least 29, at least 30, at least 31, at least 32, at least 33, at least 34 contiguous bases of, an oligonucleotide or nucleic acid disclosed herein (e.g., in the Tables), or a sequence that is complementary to a target RNA sequence gene, transcript, etc. disclosed herein, and wherein each T can be optionally and independently replaced with U and vice versa. In some embodiments, the present disclosure provides an oligonucleotide or oligonucleotide composition as disclosed herein, e.g., in a Table.

In some embodiments, an oligonucleotide is a single-stranded oligonucleotide for site-directed editing of a nucleoside (e.g., a target adenosine) in a target nucleic acid, e.g., RNA.

As described herein, oligonucleotides may contain one or more modified internucleotidic linkages (non-natural phosphate linkages). In some embodiments, a modified internucleotidic linkage is a chiral internucleotidic linkage whose linkage phosphorus is chiral. In some embodiments, a modified internucleotidic linkage is a phosphorothioate internucleotidic linkage. In some embodiments, oligonucleotides comprise one or more negatively charged internucleotidic linkages (e.g., phosphorothioate internucleotidic linkages, natural phosphate linkages, etc.). In some embodiments, oligonucleotides comprise one or more non-negatively charged internucleotidic linkage. In some embodiments, oligonucleotides comprise one or more neutral internucleotidic linkage.

In some embodiments, oligonucleotides are chirally controlled. In some embodiments, oligonucleotides are chirally pure (or “stereopure”, “stereochemically pure”), wherein the oligonucleotide exists as a single stereoisomeric form (in many cases a single diastereoisomeric (or “diastereomeric”) form as multiple chiral centers may exist in an oligonucleotide, e.g., at linkage phosphorus, sugar carbon, etc.). As appreciated by those skilled in the art, a chirally pure oligonucleotide is separated from its other stereoisomeric forms (to the extent that some impurities may exist as chemical and biological processes, selectivities and/or purifications etc. rarely, if ever, go to absolute completeness). In a chirally pure oligonucleotide, each chiral center is independently defined with respect to its configuration (for a chirally pure oligonucleotide, each internucleotidic linkage is independently stereodefined or chirally controlled). In contrast to chirally controlled and chirally pure oligonucleotides which comprise stereodefined linkage phosphorus, racemic (or “stereorandom”, “non-chirally controlled”) oligonucleotides comprising chiral linkage phosphorus, e.g., from traditional phosphoramidite oligonucleotide synthesis without stereochemical control during coupling steps in combination with traditional sulfurization (creating stereorandom phosphorothioate internucleotidic linkages), refer to a random mixture of various stereoisomers (typically diastereoisomers (or “diastereomers”) as there are multiple chiral centers in an oligonucleotide; e.g., from traditional oligonucleotide preparation using reagents containing no chiral elements other than those in nucleosides and linkage phosphorus). For example, for A*A*A wherein * is a phosphorothioate internucleotidic linkage (which comprises a chiral linkage phosphorus), a racemic oligonucleotide preparation includes four diastereomers [2²=4, considering the two chiral linkage phosphorus, each of which can exist in either of two configurations (Sp or Rp)]: A *S A *S A, A *S A *R A, A *R A *S A, and A *R A *R A, wherein *S represents a Sp phosphorothioate internucleotidic linkage and *R represents a Rp phosphorothioate internucleotidic linkage. For a chirally pure oligonucleotide, e.g., A *S A *S A, it exists in a single stereoisomeric form and it is separated from the other stereoisomers (e.g., the diastereomers A *S A *R A, A *R A *S A, and A *R A *R A).

In some embodiments, oligonucleotides comprise 1, 2, 3, 4, 5, 6, 7, 8, 9, 10 or more stereorandom internucleotidic linkages (mixture of Rp and Sp linkage phosphorus at the internucleotidic linkage, e.g., from traditional non-chirally controlled oligonucleotide synthesis). In some embodiments, oligonucleotides comprise one or more (e.g., 1-60, 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more) chirally controlled internucleotidic linkages (Rp or Sp linkage phosphorus at the internucleotidic linkage, e.g., from chirally controlled oligonucleotide synthesis). In some embodiments, an internucleotidic linkage is a phosphorothioate internucleotidic linkage. In some embodiments, an internucleotidic linkage is a stereorandom phosphorothioate internucleotidic linkage. In some embodiments, an internucleotidic linkage is a chirally controlled phosphorothioate internucleotidic linkage.

Among other things, the present disclosure provides technologies for preparing chirally controlled (in some embodiments, stereochemically pure) oligonucleotides. In some embodiments, oligonucleotides are stereochemically pure. In some embodiments, oligonucleotides of the present disclosure are about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 15%, 20%, 25%, 30%, 35%, 40%, 45%, 50%, 55%, 60%, 65%, 70%, 75%, 80%, 85%, 90%, 95%, or 100%, or at least about 5%, 10%, 15%, 20%, 25%, 30%, 35%, 40%, 45%, 50%, 55%, 60%, 65%, 70%, 75%, 80%, 85%, 90%, 95%, or 99% stereochemically pure.

In some embodiments, the present disclosure provides various oligonucleotide compositions. In some embodiments, oligonucleotide compositions are stereorandom or not chirally controlled. In some embodiments, there are no chirally controlled internucleotidic linkages in oligonucleotides of provided compositions. In some embodiments, internucleotidic linkages of oligonucleotides in compositions comprise one or more chirally controlled internucleotidic linkages (e.g., chirally controlled oligonucleotide compositions).

In some embodiments, an oligonucleotide composition comprises a plurality of oligonucleotides sharing a common base sequence, wherein one or more internucleotidic linkages in the oligonucleotides are chirally controlled and one or more internucleotidic linkages are stereorandom (not chirally controlled). In some embodiments, an oligonucleotide composition comprises a plurality of oligonucleotides sharing a common base sequence, wherein each internucleotidic linkage comprising chiral linkage phosphorus in the oligonucleotides is independently a chirally controlled internucleotidic linkage. In some embodiments, a plurality of oligonucleotides share the same base sequence, and the same base and sugar modification. In some embodiments, a plurality of oligonucleotides share the same base sequence, and the same base, sugar and internucleotidic linkage modification. In some embodiments, an oligonucleotide composition comprises oligonucleotides of the same constitution, wherein one or more internucleotidic linkages are chirally controlled and one or more internucleotidic linkages are stereorandom (not chirally controlled). In some embodiments, an oligonucleotide composition comprises oligonucleotides of the same constitution, wherein each internucleotidic linkage comprising chiral linkage phosphorus is independently a chirally controlled internucleotidic linkage. In some embodiments, at least 10%, 20%, 30%, 40%, 50%, 60%, 70%, 75%, 80%, 85%, 90% or 95% of all oligonucleotides, or all oligonucleotides of the common base sequence, are oligonucleotides of the plurality.

In some embodiments, the present disclosure provides technologies for preparing, assessing and/or utilizing provided oligonucleotides and compositions thereof.

As used in the present disclosure, in some embodiments, “one or more” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In some embodiments, “one or more” is one. In some embodiments, “one or more” is two. In some embodiments, “one or more” is three. In some embodiments, “one or more” is four. In some embodiments, “one or more” is five. In some embodiments, “one or more” is six. In some embodiments, “one or more” is seven. In some embodiments, “one or more” is eight. In some embodiments, “one or more” is nine. In some embodiments, “one or more” is ten. In some embodiments, “one or more” is at least one. In some embodiments, “one or more” is at least two. In some embodiments, “one or more” is at least three. In some embodiments, “one or more” is at least four. In some embodiments, “one or more” is at least five. In some embodiments, “one or more” is at least six. In some embodiments, “one or more” is at least seven. In some embodiments, “one or more” is at least eight. In some embodiments, “one or more” is at least nine. In some embodiments, “one or more” is at least ten.

As used in the present disclosure, in some embodiments, “at least one” is one or more.

### Oligonucleotides

Among other things, the present disclosure provides oligonucleotides of various designs, which may comprise various nucleobases and patterns thereof, sugars and patterns thereof, internucleotidic linkages and patterns thereof, and/or additional chemical moieties and patterns thereof as described in the present disclosure. In some embodiments, provided oligonucleotides can direct A to I editing in target nucleic acids. In some embodiments, oligonucleotides of the present disclosure are single-stranded oligonucleotides capable of site-directed editing of an adenosine (coversion of A into I) in a target RNA sequence.

In some embodiments, oligonucleotides are of suitable lengths and sequence complementarity to specifically hybridize with target nucleic acids. In some embodiments, oligonucleotide is sufficiently long and is sufficiently complementary to target nucleic acids to distinguish target nucleic acid from other nucleic acids to reduce off-target effects. In some embodiments, oligonucleotide is sufficiently short to facilitate delivery, reduce manufacture complexity and/or cost which maintaining desired properties and activities (e.g., editing of adenosine).

In some embodiments, an oligonucleotide has a length of about 10-200 (e.g., about 10-20, 10-30, 10-40, 10-50, 10-60, 10-70, 10-80, 10-90, 10-100, 10-120, 10-150, 20-30, 20-40, 20-50, 20-60, 20-70, 20-80, 20-90, 20-100, 20-120, 20-150, 20-200, 25-30, 25-40, 25-50, 25-60, 25-70, 25-80, 25-90, 25-100, 25-120, 25-150, 25-200, 30-40, 30-50, 30-60, 30-70, 30-80, 30-90, 30-100, 30-120, 30-150, 30-200, 10, 20, 25, 26, 27, 28, 29, 30, 31, 32, 33, 34, 35, 36, 37, 38, 39, 40, 45, 50, 60, etc.) nucleobases. In some embodiments, the base sequence of an oligonucleotide is about 10-60 nucleobases in length. In some embodiments, a base sequence is about 15-50 nucleobases in length. In some embodiments, a base sequence is from about 15 to about 35 nucleobases in length. In some embodiments, a base sequence is from about 25 to about 34 nucleobases in length. In some embodiments, a base sequence is from about 26 to about 35 nucleobases in length. In some embodiments, a base sequence is from about 27 to about 32 nucleobases in length. In some embodiments, a base sequence is from about 29 to about 35 nucleobases in length. In some embodiments, a base sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleobases in length. In some other embodiments, a base sequence is or is at least 35 nucleobases in length. In some other embodiments, a base sequence is or is at least 34 nucleobases in length. In some other embodiments, a base sequence is or is at least 33 nucleobases in length. In some other embodiments, a base sequence is or is at least 32 nucleobases in length. In some other embodiments, a base sequence is or is at least 31 nucleobases in length. In some other embodiments, a base sequence is or is at least 30 nucleobases in length. In some other embodiments, a base sequence is or is at least 29 nucleobases in length. In some other embodiments, a base sequence is or is at least 28 nucleobases in length. In some other embodiments, a base sequence is or is at least 27 nucleobases in length. In some other embodiments, a base sequence is or is at least 26 nucleobases in length. In some other embodiments, the base sequence of the complementary portion in a duplex is at least 15, 16, 17, 18, 19, 20, 21, 22, 23, 24, 25, 16, 27, 28, 29, 30, 31, 32, 33, 34, 35 or more nucleobases in length. In some other embodiments, it is at least 18 nucleobases in length. In some other embodiments, it is at least 19 nucleobases in length. In some other embodiments, it is at least 20 nucleobases in length. In some other embodiments, it is at least 21 nucleobases in length. In some other embodiments, it is at least 22 nucleobases in length. In some other embodiments, it is at least 23 nucleobases in length. In some other embodiments, it is at least 24 nucleobases in length. In some other embodiments, it is at least 25 nucleobases in length. Among other things, the present disclosure provides oligonucleotides of comparable or better properties and/or comparable or higher activities but of shorter lengths compared to prior reported adenosine editing oligonucleotides.

In some embodiments, a base sequence of the oligonucleotide is complementary to a base sequence of a target nucleic acid (e.g., complementarity to a portion of the target nucleic acid comprising the target adenosine) with 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) mismatches which are not Watson-Crick base pairs (AT, AU and CG). In some embodiments, there are no mismatches. In some embodiments, there is 1 mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches. In some embodiments, oligonucleotides may contain portions that are not designed for complementarity (e.g., loops, protein binding sequences, etc., for recruiting of proteins, e.g., ADAR). As those skilled in the art will appreciate, when calculating mismatches and/or complementarity, such portions may be properly excluded. In some embodiments, complementarity, e.g., between oligonucleotides and target nucleic acids, is about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.). In some embodiments, complementarity is at least about 60%. In some embodiments, complementarity is at least about 65%. In some embodiments, complementarity is at least about 70%. In some embodiments, complementarity is at least about 75%. In some embodiments, complementarity is at least about 80%. In some embodiments, complementarity is at least about 85%. In some embodiments, complementarity is at least about 90%. In some embodiments, complementarity is at least about 95%. In some embodiments, complementarity is 100% across the length of an oligonucleotide. In some embodiments, complementarity is 100% except at a nucleoside opposite to a target nucleoside (e.g., adenosine) across the length of an oligonucleotide. Typically, complementarity is based on Watson-Crick base pairs AT, AU and CG. Those skilled in the art will appreciate that when assessing complementarity of two sequences of different lengths (e.g., a provided oligonucleotide and a target nucleic acid) complementarity may be properly based on the length of the shorter sequence and/or maximum complementarity between the two sequences. In many embodiments, oligonucleotides and target nucleic acids are of sufficient complementarity such that modifications are selectively directed to target adenosine sites.

In some embodiments, one or more mismatches are independently wobbles. In some embodiments, each mismatch is a wobble. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) wobbles. In some embodiments, the number is 0. In some embodiments, the number is 1. In some embodiments, the number is 2. In some embodiments, the number is 3. In some embodiments, the number is 4. In some embodiments, the number is 5. In some embodiments, a wobble is G-U, I-A, G-A, I-U, I-C, I-T, A-A, or reverse A-T. In some embodiments, a wobble is G-U, I-A, G-A, I-U, or I-C. In some embodiments, I-C may be considered a match when I is a 3′ immediate nucleoside next to a nucleoside opposite to a target nucleoside.

In some embodiments, duplexes of oligonucleotides and target nucleic acids comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, distances between two mismatches, mismatches and one or both ends of oligonucleotides (or a portion thereof, e.g., first domain, second domain, first subdomain, second subdomain, third subdomain), and/or mismatches and nucleosides opposite to target adenosine can independently be 0-50, 0-40, 0-30, 0-25, 0-20, 0-15, 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleobases (not including mismatches, end nucleosides and nucleosides opposite to target adenosine). In some embodiments, a number is 0-30. In some embodiments, a number is 0-20. In some embodiments, a number is 1, 2, 3, 4, 5, 6, 7, 8, 9, 10, 11, 12, 13, 14, 15, 16, 17, 18, 19, or 20. In some embodiments, a distance between two mismatches is 0-20. In some embodiments, a distance between two mismatches is 1-10. In some embodiments, a distance between a mismatch and a 5′-end nucleoside of an oligonucleotide is 0-20. In some embodiments, a distance between a mismatch and a 5′-end nucleoside of an oligonucleotide is 5-20. In some embodiments, a distance between a mismatch and a 3′-end nucleoside of an oligonucleotide is 0-40. In some embodiments, a distance between a mismatch and a 3′-end nucleoside of an oligonucleotide is 5-20. In some embodiments, a distance between a mismatch and a nucleoside opposite to a target adenosine is 0-20. In some embodiments, a distance between a mismatch and a nucleoside opposite to a target adenosine is 1-10. In some embodiments, the number of nucleobases for a distance is 0. In some embodiments, it is 1. In some embodiments, it is 2. In some embodiments, it is 3. In some embodiments, it is 4. In some embodiments, it is 5. In some embodiments, it is 6. In some embodiments, it is 7. In some embodiments, it is 8. In some embodiments, it is 9. In some embodiments, it is 10. In some embodiments, it is 11. In some embodiments, it is 12. In some embodiments, it is 13. In some embodiments, it is 14. In some embodiments, it is 15. In some embodiments, it is 16. In some embodiments, it is 17. In some embodiments, it is 18. In some embodiments, it is 19. In some embodiments, it is 20. In some embodiments, a mismatch is at an end, e.g., a 5′-end or 3′-end of a first domain, second domain, first subdomain, second subdomain, or third subdomain. In some embodiments, a mismatch is at a nucleoside opposite to a target adenosine.

In some embodiments, provided oligonucleotides can direct adenosine editing (e.g., converting A to I) in a target nucleic acid and has a base sequence which consists of, comprises, or comprises a portion (e.g., a span of 10, 11, 12, 13, 14, 15, 16, 17, 18, 19 or more contiguous bases) of the base sequence of an oligonucleotide disclosed herein, wherein each T can be independently replaced with U and vice versa, and the oligonucleotide comprises at least one non-naturally-occurring modification of a base, sugar and/or internucleotidic linkage.

In some embodiments, a provided oligonucleotide comprises one or more carbohydrate moieties. In some embodiments, a provided oligonucleotide comprises one or more GalNAc moieties. In some embodiments, a provided oligonucleotide comprises one or more targeting moieties. Non-limiting examples of such additional chemical moieties which can be conjugated to oligonucleotide chain are described herein.

In some embodiments, provided oligonucleotides can direct a correction of a G to A mutation in a target sequence, or a product thereof. In some embodiments, a correction of a G to A mutation is or comprises conversion of A to I, which can be read as G during translation or other biological processes. In some embodiments, provided oligonucleotides can direct a correction of a G to A mutation in a target sequence or a product thereof via ADAR-mediated deamination. In some embodiments, provided oligonucleotides can direct a correction of a G to A mutation in a target sequence or a product thereof via ADAR-mediated deamination by recruiting an endogenous ADAR (e.g., in a target cell) and facilicating the ADAR-mediated deamination. Regardless, however, the present disclosure is not limited to any particular mechanism. In some embodiments, the present disclosure provides oligonucleotides, compositions, methods, etc., capable of operating via double-stranded RNA interference, single-stranded RNA interference, RNase H-mediated knock-down, steric hindrance of translation, ADAR-meidated deamination or a combination of two or more such mechanisms.

In some embodiments, an oligonucleotide comprises a structural element or a portion thereof described herein, e.g., in a Table. In some embodiments, an oligonucleotide has a base sequence which comprises the base sequence (or a portion thereof) wherein each T can be independently substituted with U, pattern of chemical modifications (or a portion thereof), and/or a format of an oligonucleotide disclosed herein, e.g., in a Table or in the Figures, or otherwise disclosed herein. In some embodiments, such oligonucleotide can direct a correction of a G to A mutation in a target sequence, or a product thereof.

Among other things, provided oligonucleotides may hybridize to their target nucleic acids (e.g., pre-mRNA, mature mRNA, etc.). In some embodiments, oligonucleotide can hybridize to a target RNA sequence nucleic acid in any stage of RNA processing, including but not limited to a pre-mRNA or a mature mRNA. In some embodiments, oligonucleotide can hybridize to any element of oligonucleotide nucleic acid or its complement, including but not limited to: a promoter region, an enhancer region, a transcriptional stop region, a translational start signal, a translation stop signal, a coding region, a non-coding region, an exon, an intron, an intron/exon or exon/intron junction, the 5′ UTR, or the 3′ UTR.

In some embodiments, oligonucleotide hybridizes to two or more variants of transcripts derived from a sense strand of a target site (e.g., a target sequence).

In some embodiments, provided oligonucleotides contain increased levels of one or more isotopes. In some embodiments, provided oligonucleotides are labeled, e.g., by one or more isotopes of one or more elements, e.g., hydrogen, carbon, nitrogen, etc. In some embodiments, provided oligonucleotides in provided compositions, e.g., oligonucleotides of a plurality of a composition, comprise base modifications, sugar modifications, and/or internucleotidic linkage modifications, wherein the oligonucleotides contain an enriched level of deuterium. In some embodiments, provided oligonucleotides are labeled with deuterium (replacing —¹H with —²H) at one or more positions. In some embodiments, one or more ¹H of an oligonucleotide chain or any moiety conjugated to the oligonucleotide chain (e.g., a targeting moiety, etc.) is substituted with ²H. Such oligonucleotides can be used in compositions and methods described herein.

In some embodiments, oligonucleotides comprise one or more modified nucleobases, one or more modified sugars, and/or one or more modified internucleotidic linkages as described herein. In some embodiments, oligonucleotides comprise a certain level of modified nucleobases, modified sugars, and/or modified internucleotidic linkages, e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all nucleobases, sugars, and internucleotidic linkages, respectively, within an oligonucleotide.

In some embodiments, oligonucleotides comprise one or more modified sugars. In some embodiments, oligonucleotides of the present disclosure comprise one or more modified nucleobases. Various modifications can be introduced to a sugar and/or nucleobase in accordance with the present disclosure. For example, in some embodiments, a modification is a modification described in U.S. Pat. No. 9,006,198. In some embodiments, a modification is a modification described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, WO 2019/032612, and/or WO 2020/191252, the sugars, bases, and internucleotidic linkages of each of which are independently incorporated herein by reference.

In some embodiments, a nucleobase in a nucleoside is or comprises Ring BA which has the structure of BA-I, BA-I-a, BA-I-b, BA-II, BA-II-a, BA-II-b, BA-III, BA-III-a, BA-III-b, BA-IV, BA-IV-a, BA-IV-b, BA-V, BA-V-a, BA-V-b, or BA-VI, or a tautomer of Ring BA, wherein the nucleobase is optionally substituted or protected.

In some embodiments, a sugar is a modified sugar comprising a 2′-modificatin, e.g., 2′-F, 2′-OR wherein R is optionally substituted aliphatic, or a bicyclic sugar (e.g., a LNA sugar), or a acyclic sugar (e.g., a UNA sugar).

In some embodiments, as described herein, provided oligonucleotides comprise one or more domains, each of which independently has certain lengths, modifications, linkage phosphorus stereochemistry, etc., as described herein. In some embodiments, the present disclosure provides an oligonucleotide comprising one or more modified sugars and/or one or more modified internucleotidic linkages, wherein the oligonucleotide comprises a first domain and a second domain each independently comprising one or more nucleobases. In some embodiments, the present disclosure provides an oligonucleotide comprising:

a first domain; and

a second domain, wherein:

the first domain comprises one or more 2′-F modifications;

the second domain comprises one or more sugars that do not have a 2′-F modification.

In some embodiments, an oligonucleotide or a portion thereof (e.g., a first domain, a second domain, a first subdomain, a second subdomain, a third subdomain, etc.) comprises a certain level of modified sugars. In some embodiments, a modified sugar comprises a 2′-modification. In some embodiments, a modified sugar is a bicyclic sugar. In some embodiments, a modified sugar is an acyclic sugar (e.g., by breaking a C2-C3 bond of a corresponding cyclic sugar). In some embodiments, a modified sugar comprises a 5′-modification. Typically, oligonucleotides of the present disclosure have a free 5′-OH at its 5′-end and a free 3′-OH at its 3′-end unless indicated otherwise, e.g., by context. In some embodiments, a 5′-end sugar of an oligonucleotide may comprise a modified 5′-OH.

In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all sugars in an oligonucleotide or a portion thereof, respectively. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%.

In some embodiments, an oligonucleotide or a portion thereof (e.g., a first domain, a second domain, a first subdomain, a second subdomain, a third subdomain, etc.) comprises a certain level of modified internucleotidic linkages. In some embodiments, an oligonucleotide or a portion thereof (e.g., a first domain, a second domain, a first subdomain, a second subdomain, a third subdomain, etc.) comprises a certain level of chiral internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in an oligonucleotide or a portion thereof, respectively. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%.

In some embodiments, an oligonucleotide or a portion thereof (e.g., a first domain, a second domain, a first subdomain, a second subdomain, a third subdomain, etc.) comprises a certain level of chirally controlled internucleotidic linkages. In some embodiments, an oligonucleotide or a portion thereof (e.g., a first domain, a second domain, a first subdomain, a second subdomain, a third subdomain, etc.) comprises a certain level of Sp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in an oligonucleotide or a portion thereof, respectively. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in an oligonucleotide or a portion thereof, respectively. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%.

In some embodiments, an oligonucleotide or a portion thereof (e.g., a first domain, a second domain, a first subdomain, a second subdomain, a third subdomain, etc.) comprises a certain level of Sp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in an oligonucleotide or a portion thereof, respectively. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in an oligonucleotide or a portion thereof, respectively. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in an oligonucleotide or a portion thereof, respectively. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, about 1-50, 1-40, 1-30, e.g., about 1, 2, 3, 4, 5, 6, 7, 8, 9, 10, 11, 12, 13, 14, 15, 16, 17, 18, 19, or 20 internucleotidic linkages are independently Sp chiral internucleotidic linkages. In many embodiments, it was observed that a high percentage (e.g., relative to Rp internucleotidic linkages and/or natural phosphate linkages) of Sp internucleotidic linkages in an oligonucleotide or certain portions thereof can provide improved properties and/or activities, e.g., high stability and/or high adenosine editing activity.

In some embodiments, an oligonucleotide or a portion thereof (e.g., a first domain, a second domain, a first subdomain, a second subdomain, a third subdomain, etc.) comprises a certain level of Rp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in an oligonucleotide or a portion thereof, respectively. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in an oligonucleotide or a portion thereof, respectively. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in an oligonucleotide or a portion thereof, respectively. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, about 1-50, 1-40, 1-30, e.g., about 1, 2, 3, 4, 5, 6, 7, 8, 9, 10, 11, 12, 13, 14, 15, 16, 17, 18, 19, or 20 internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10.

While not wishing to be bound by theory, it is noted that in some instances Rp and Sp configurations of internucleotidic linkages may affect structural changes in helical conformations of double stranded complexes formed by oligonucleotides and target nucleic acids such as RNA, and ADAR proteins may recognize and interact various targets (e.g., double stranded complexes formed by oligonucleotides and target nucleic acids such as RNA) through multiple domains. In some embodiments, provided oligonucleotides and compositions thereof promote and/or enhance interaction profiles of oligonucleotide, target nucleic acids, and/or ADAR proteins to provide efficient adenosine modification by ADAR proteins through incorporation of various modifications and/or control of stereochemistry.

In some embodiments, an oligonucleotide can have or comprise a base sequence; internucleotidic linkage, base modification, sugar modification, additional chemical moiety, or pattern thereof; and/or any other structural element described herein, e.g., in Tables.

In some embodiments, a provided oligonucleotide or composition is characterized in that, when it is contacted with a target nucleic acid comprising a target adenosine in a system (e.g., an ADAR-mediated deamination system), modification of the target adenosine (e.g., deamination of the target A) is improved relative to that observed under reference conditions (e.g., selected from the group consisting of absence of the composition, presence of a reference oligonucleotide or composition, and combinations thereof). In some embodiments, modification, e.g., ADAR-mediated deamination (e.g., endogenous ADAR-mediated deamination) is increased 10%, 20%, 30%, 40%, 50%, 60%, 70%, 80%, 90%, 100%, or 2, 3, 4, 5, 6, 7, 8, 9, 10, 11, 12, 13, 14, 15, 16, 17, 18,19, 20, 21, 22, 23, 24, 25, 26, 27, 28, 29, 30, 40, 50, 60, 70, 80, 90, 100, 200, 300, 400, 500, 600, 700, 800, 900, 1000 fold or more.

In some embodiments, oligonucleotides are provided as salt forms. In some embodiments, oligonucleotides are provided as salts comprising negatively-charged internucleotidic linkages (e.g., phosphorothioate internucleotidic linkages, natural phosphate linkages, etc.) existing as their salt forms. In some embodiments, oligonucleotides are provided as pharmaceutically acceptable salts. In some embodiments, oligonucleotides are provided as metal salts. In some embodiments, oligonucleotides are provided as sodium salts. In some embodiments, oligonucleotides are provided as ammonium salts. In some embodiments, oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

In some embodiments, oligonucleotides are chiral controlled, comprising one or more chirally controlled internucleotidic linkages. In some embodiments, provided oligonucleotides are stereochemically pure. In some embodiments, provided oligonucleotides or compositions thereof are substantially pure of other stereoisomers. In some embodiments, the present disclosure provides chirally controlled oligonucleotide compositions.

As described herein, oligonucleotides of the present disclosure can be provided in high purity (e.g., 50%-100%). In some embodiments, oligonucleotides of the present disclosure are of high stereochemical purity (e.g., 50%-100%). In some embodiments, oligonucleotides in provided compositions are of high stereochemical purity (e.g., high percentage (e.g., 50%-100%) of a stereoisomer compared to the other stereoisomers of the same oligonucleotide). In some embodiments, a percentage is at least or about 50%. In some embodiments, a percentage is at least or about 60%. In some embodiments, a percentage is at least or about 70%. In some embodiments, a percentage is at least or about 75%. In some embodiments, a percentage is at least or about 80%. In some embodiments, a percentage is at least or about 85%. In some embodiments, a percentage is at least or about 90%. In some embodiments, a percentage is at least or about 95%.

### First Domains

As described herein, in some embodiment, an oligonucleotide comprises a first domain and a second domain. In some embodiments, an oligonucleotide consists of a first domain and a second domain. Certain embodiments are described below as examples.

In some embodiments, a first domain has a length of about 2-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases. In some embodiments, a first domain has a length of about 5-30 nucleobases. In some embodiments, a first domain has a length of about 10-30 nucleobases. In some embodiments, a first domain has a length of about 10-20 nucleobases. In some embodiments, a first domain has a length of about 13-16 nucleobases. In some embodiments, a first domain has a length of 10 nucleobases. In some embodiments, a first domain has a length of 11 nucleobases. In some embodiments, a first domain has a length of 12 nucleobases. In some embodiments, a first domain has a length of 13 nucleobases. In some embodiments, a first domain has a length of 14 nucleobases. In some embodiments, a first domain has a length of 15 nucleobases. In some embodiments, a first domain has a length of 16 nucleobases. In some embodiments, a first domain has a length of 17 nucleobases. In some embodiments, a first domain has a length of 18 nucleobases. In some embodiments, a first domain has a length of 19 nucleobases. In some embodiments, a first domain has a length of 20 nucleobases.

In some embodiments, a first domain is about, or at least about, 5-95%, 10%-90%, 20%-80%, 30%-70%, 40%-70%, 40%-60%, 5%, 10%, 15%, 20%, 25%, 30%, 35%, 40%, 45%, 50%, 55%, 60%, 65%, 70%, 75%, 80%, 85%, 90%, 95% of an oligonucleotide. In some embodiments, a percentage is about 30%-80%. In some embodiments, a percentage is about 30%-70%. In some embodiments, a percentage is about 40%-60%. In some embodiments, a percentage is about 20%. In some embodiments, a percentage is about 25%. In some embodiments, a percentage is about 30%. In some embodiments, a percentage is about 35%. In some embodiments, a percentage is about 40%. In some embodiments, a percentage is about 45%. In some embodiments, a percentage is about 50%. In some embodiments, a percentage is about 55%. In some embodiments, a percentage is about 60%. In some embodiments, a percentage is about 65%. In some embodiments, a percentage is about 70%. In some embodiments, a percentage is about 75%. In some embodiments, a percentage is about 80%. In some embodiments, a percentage is about 85%. In some embodiments, a percentage is about 90%.

In some embodiments, one or more (e.g., 1-20, 1, 2, 3, 4, 5, 6, 7, 8, 9, or 10, etc.) mismatches exist in a first domain when an oligonucleotide is aligned with a target nucleic acid for complementarity. In some embodiments, there is 1 mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches.

In some embodiments, one or more (e.g., 1-20, 1, 2, 3, 4, 5, 6, 7, 8, 9, or 10, etc.) wobbles exist in a first domain when an oligonucleotide is aligned with a target nucleic acid for complementarity. In some embodiments, there is 1 wobble. In some embodiments, there are 2 wobbles. In some embodiments, there are 3 wobbles. In some embodiments, there are 4 wobbles. In some embodiments, there are 5 wobbles. In some embodiments, there are 6 wobbles. In some embodiments, there are 7 wobbles. In some embodiments, there are 8 wobbles. In some embodiments, there are 9 wobbles. In some embodiments, there are 10 wobbles.

In some embodiments, duplexes of oligonucleotides and target nucleic acids in a first domain region comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, a first domain is fully complementary to a target nucleic acid.

In some embodiments, a first domain comprises one or more modified nucleobases.

In some embodiments, a second domain comprises one or more sugars comprising two 2′-H (e.g., natural DNA sugars). In some embodiments, a second domain comprises one or more sugars comprising 2′-OH (e.g., natural RNA sugars). In some embodiments, a first domain comprises one or more modified sugars. In some embodiments, a modified sugar comprises a 2′-modification. In some embodiments, a modified sugar is a bicyclic sugar, e.g., a LNA sugar. In some embodiments, a modified sugar is an acyclic sugar (e.g., by breaking a C2-C3 bond of a corresponding cyclic sugar).

In some embodiments, a first domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars. In some embodiments, a first domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars with 2′-F modification.

In some embodiments, about 5%-100%, (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a first domain are independently a modified sugar. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a first domain are independently a 2′-F modified sugar. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%.

In some embodiments, a first domain comprises no bicyclic sugars or 2′-OR modified sugars wherein R is not —H. In some embodiments, a first domain comprises one or more (e.g., 1, 2, 3, 4, 5, 6, 7, 8, 9, or 10) bicyclic sugars and/or 2′-OR modified sugars wherein R is not —H. In some embodiments, a first domain comprises one or more (e.g., 1, 2, 3, 4, 5, 6, 7, 8, 9, or 10) 2′-OR modified sugars wherein R is not —H. In some embodiments, a first domain comprises one or more (e.g., 1, 2, 3, 4, 5, 6, 7, 8, 9, or 10) 2′-OR modified sugars wherein R is optionally substituted C₁₋₁₀ aliphatic. In some embodiments, levels of bicyclic sugars and/or 2′-OR modified sugars wherein R is not —H, individually or combined, are relatively low compared to level of 2′-F modified sugars. In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a first domain comprises 2′-OMe. In some embodiments, no more than about 50% of sugars in a first domain comprises 2′-OMe. In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a first domain comprises 2′-OR, wherein R is optionally substituted C₁₋₆ aliphatic. In some embodiments, no more than about 50% of sugars in a first domain comprises 2′-OR, wherein R is optionally substituted C₁₋₆ aliphatic. In some embodiments, no more than about 40% of sugars in a first domain comprises 2′-OR, wherein R is optionally substituted C₁₋₆ aliphatic. In some embodiments, no more than about 30% of sugars in a first domain comprises 2′-OR, wherein R is optionally substituted C₁₋₆ aliphatic. In some embodiments, no more than about 25% of sugars in a first domain comprises 2′-OR, wherein R is optionally substituted C₁₋₆ aliphatic. In some embodiments, no more than about 20% of sugars in a first domain comprises 2′-OR, wherein R is optionally substituted C₁₋₆ aliphatic. In some embodiments, no more than about 10% of sugars in a first domain comprises 2′-OR, wherein R is optionally substituted C₁₋₆ aliphatic. In some embodiments, as described herein, 2′-OR is 2′-MOE. In some embodiments, as described herein, 2′-OR is 2′-MOE or 2′-OMe. In some embodiments, a first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification. In some embodiments, a first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification. In some embodiments, a first domain comprises one or more (e.g., about 1-20, 1, 2, 3, 4, 5, 6, 7, 8, 9, 10, 11, 12, 13, 14, 15, 16, 17, 18, 19, 20, etc.) bicyclic sugars, e.g., LNA sugars. In some embodiments, a first domain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars). In some embodiments, a number of 5′-end sugars in a first domain are independently 2′-OR modified sugars, wherein R is not —H. In some embodiments, a number of (e.g., 1, 2, 3, 4, 5, 6, 7, 8, 9, 10 or more) 5′-end sugars in a first domain are independently 2′-OR modified sugars, wherein R is independently optionally substituted C₁₋₆ aliphatic. In some embodiments, the first about 1-10, e.g., 1, 2, 3, 4, 5, 6, 7, 8, 9, or 10, sugars from the 5′-end of a first domain are independently 2′-OR modified sugars, wherein R is independently optionally substituted C₁₋₆ aliphatic. In some embodiments, the first one is 2′-OR modified. In some embodiments, the first two are independently 2′-OR modified. In some embodiments, the first three are independently 2′-OR modified. In some embodiments, the first four are independently 2′-OR modified. In some embodiments, the first five are independently 2′-OR modified. In some embodiments, all 2′-OR modification in a domain (e.g., a first domain), a subdomain (e.g., a first subdomain), or an oligonucleotide are the same. In some embodiments, 2′-OR is 2′-MOE. In some embodiments, 2′-OR is 2′-OMe.

In some embodiments, no sugar in a first domain comprises 2′-OR. In some embodiments, no sugar in a first domain comprises 2′-OMe. In some embodiments, no sugar in a first domain comprises 2′-MOE. In some embodiments, no sugar in a first domain comprises 2′-MOE or 2′-OMe. In some embodiments, no sugar in a first domain comprises 2′-OR, wherein R is optionally substituted C₁₋₆ aliphatic. In some embodiments, each sugar in a first domain comprises 2′-F.

In some embodiments, a first domain comprise about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in a first domain are modified internucleotidic linkages. In some embodiments, each internucleotidic linkage in a first domain is independently a modified internucleotidic linkage. In some embodiments, each modified internucleotidic linkages is independently a chiral internucleotidic linkage. In some embodiments, a modified or chiral internucleotidic linkage is a phosphorothioate internucleotidic linkage. In some embodiments, a modified or chiral internucleotidic linkage is a non-negatively charged internucleotidic linkage. In some embodiments, a modified or chiral internucleotidic linkage is a neutral internucleotidic linkage, e.g., n001. In some embodiments, each modified internucleotidic linkages is independently a phosphorothioate internucleotidic linkage or a non-negatively charged internucleotidic linkage. In some embodiments, each modified internucleotidic linkages is independently a phosphorothioate internucleotidic linkage or a neutral internucleotidic linkage. In some embodiments, each modified internucleotidic linkages is independently a phosphorothioate internucleotidic linkage. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a first domain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a first domain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a first domain is chirally controlled. In some embodiments, each is independently chirally controlled. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a first domain is Sp. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) phosphorothioate internucleotidic linkages in a first domain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a first domain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a first domain is Sp. In some embodiments, the number is one or more. In some embodiments, the number is 2 or more. In some embodiments, the number is 3 or more. In some embodiments, the number is 4 or more. In some embodiments, the number is 5 or more. In some embodiments, the number is 6 or more. In some embodiments, the number is 7 or more. In some embodiments, the number is 8 or more. In some embodiments, the number is 9 or more. In some embodiments, the number is 10 or more. In some embodiments, the number is 11 or more. In some embodiments, the number is 12 or more. In some embodiments, the number is 13 or more. In some embodiments, the number is 14 or more. In some embodiments, the number is 15 or more. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, each internucleotidic linkages linking two first domain nucleosides is independently a modified internucleotidic linkage. In some embodiments, each modified internucleotidic linkages is independently a chiral internucleotidic linkage. In some embodiments, each modified internucleotidic linkages is independently a phosphorothioate internucleotidic linkage. In some embodiments, each chiral internucleotidic linkage is independently a phosphorothioate internucleotidic linkage. In some embodiments, each modified internucleotidic linkages is independently a Sp chiral internucleotidic linkage. In some embodiments, each modified internucleotidic linkages is independently a Sp phosphorothioate internucleotidic linkage. In some embodiments, each chiral internucleotidic linkages is independently a Sp phosphorothioate internucleotidic linkage. In some embodiments, an internucleotidic linkage of a first domain is bonded to two nucleosides of the first domain. In some embodiments, an internucleotidic linkage bonded to a nucleoside in a first domain and a nucleoside in a second domain may be properly considered an internucleotidic linkage of a first domain. In some embodiments, an internucleotidic linkage bonded to a nucleoside in a first domain and a nucleoside in a second domain is a modified internucleotidic linkage; in some embodiments, it is a chiral internucleotidic linkage; in some embodiments, it is chirally controlled; in some embodiments, it is Rp; in some embodiments, it is Sp. In many embodiments, it was observed that a high percentage (e.g., relative to Rp internucleotidic linkages and/or natural phosphate linkages) of Sp internucleotidic linkages provide improved properties and/or activities, e.g., high stability and/or high adenosine editing activity.

In some embodiments, a first domain comprises a certain level of Rp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in a first domain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in a first domain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in a first domain. In some embodiments, a percentage is about or no more than about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, about 1-50, 1-40, 1-30, 1-25, 1-20, 1-15, 1-10, 1-5, e.g., about 1, 2, 3, 4, 5, 6, 7, 8, 9, 10, 11, 12, 13, 14, 15, 16, 17, 18, 19, or 20 internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10.

In some embodiments, each phosphorothioate internucleotidic linkage in a first domain is independently chirally controlled. In some embodiments, each is independently Sp or Rp. In some embodiments, a high level is Sp as described herein. In some embodiments, each phosphorothioate internucleotidic linkage in a first domain is chirally controlled and is Sp.

In some embodiments, as illustrated in certain examples, a first domain comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in a first domain is about 1-10, or about 1, 2, 3, 4, 5, 6, 7, 8, 9, or 10. In some embodiments, it is about 1. In some embodiments, it is about 2. In some embodiments, it is about 3. In some embodiments, it is about 4. In some embodiments, it is about 5. In some embodiments, two or more non-negatively charged internucleotidic linkages are consecutive. In some embodiments, no two non-negatively charged internucleotidic linkages are consecutive. In some embodiments, all non-negatively charged internucleotidic linkages in a first domain are consecutive (e.g., 3 consecutive non-negatively charged internucleotidic linkages). In some embodiments, a non-negatively charged internucleotidic linkage, or two or more consecutive non-negatively charged internucleotidic linkages, are at the 5′-end of a first domain. In some embodiments, the internucleotidic linkage linking the last two nucleosides of a first domain is a non-negatively charged internucleotidic linkage. In some embodiments, the internucleotidic linkage linking the last two nucleosides of a first domain is a Sp non-negatively charged internucleotidic linkage. In some embodiments, the internucleotidic linkage linking the last two nucleosides of a first domain is a Rp non-negatively charged internucleotidic linkage. In some embodiments, the internucleotidic linkage linking the last two nucleosides of a first domain is a phosphorothioate internucleotidic linkage. In some embodiments, the internucleotidic linkage linking the last two nucleosides of a first domain is a Sp phosphorothioate internucleotidic linkage. In some embodiments, the internucleotidic linkage linking the first two nucleosides of a first domain is a non-negatively charged internucleotidic linkage. In some embodiments, the internucleotidic linkage linking the first two nucleosides of a first domain is a Sp non-negatively charged internucleotidic linkage. In some embodiments, the internucleotidic linkage linking the first two nucleosides of a first domain is a Rp non-negatively charged internucleotidic linkage. In some embodiments, the internucleotidic linkage linking the first two nucleosides of a first domain is a phosphorothioate internucleotidic linkage. In some embodiments, the internucleotidic linkage linking the first two nucleosides of a first domain is a Sp phosphorothioate internucleotidic linkage. In some embodiments, a non-negatively charged internucleotidic linkage is a neutral internucleotidic linkage such as n001. In some embodiments, the first two nucleosides of a first domain are the first two nucleosides of an oligonucleotide.

In some embodiments, a first domain comprises one or more natural phosphate linkages. In some embodiments, a first domain contains no natural phosphate linkages.

In some embodiments, a first domain recruits, promotes or contribute to recruitment of, a protein such as an ADAR protein (e.g., ADAR1, ADAR2, etc.). In some embodiments, a first domain recruits, or promotes or contribute to interactions with, a protein such as an ADAR protein. In some embodiments, a first domain contacts with a RNA binding domain (RBD) of ADAR. In some embodiments, a first domain does not substantially contact with a second RBD domain of ADAR. In some embodiments, a first domain does not substantially contact with a catalytic domain of ADAR which has a deaminase activity. In some embodiments, various nucleobases, sugars and/or internucleotidic linkages may interact with one or more residues of proteins, e.g., ADAR proteins.

### Second Domains

As described herein, in some embodiment, an oligonucleotide comprises a first domain and a second domain from 5′ to 3′. In some embodiments, an oligonucleotide consists of a first domain and a second domain. Certain embodiments of a second domain are described below as examples. In some embodiments, a second domain comprise a nucleoside opposite to a target adenosine to be modified (e.g., conversion to I).

In some embodiments, a second domain has a length of about 2-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases. In some embodiments, a second domain has a length of about 5-30 nucleobases. In some embodiments, a second domain has a length of about 10-30 nucleobases. In some embodiments, a second domain has a length of about 10-20 nucleobases. In some embodiments, a second domain has a length of about 5-15 nucleobases. In some embodiments, a second domain has a length of about 13-16 nucleobases. In some embodiments, a second domain has a length of about 1-7 nucleobases. In some embodiments, a second domain has a length of 10 nucleobases. In some embodiments, a second domain has a length of 11 nucleobases. In some embodiments, a second domain has a length of 12 nucleobases. In some embodiments, a second domain has a length of 13 nucleobases. In some embodiments, a second domain has a length of 14 nucleobases. In some embodiments, a second domain has a length of 15 nucleobases. In some embodiments, a second domain has a length of 16 nucleobases. In some embodiments, a second domain has a length of 17 nucleobases. In some embodiments, a second domain has a length of 18 nucleobases. In some embodiments, a second domain has a length of 19 nucleobases. In some embodiments, a second domain has a length of 20 nucleobases.

In some embodiments, a second domain is about, or at least about, 5-95%, 10%-90%, 20%-80%, 30%-70%, 40%-70%, 40%-60%, 5%, 10%, 15%, 20%, 25%, 30%, 35%, 40%, 45%, 50%, 55%, 60%, 65%, 70%, 75%, 80%, 85%, 90%, 95% of an oligonucleotide. In some embodiments, a percentage is about 30%-80%. In some embodiments, a percentage is about 30%-70%. In some embodiments, a percentage is about 40%-60%. In some embodiments, a percentage is about 20%. In some embodiments, a percentage is about 25%. In some embodiments, a percentage is about 30%. In some embodiments, a percentage is about 35%. In some embodiments, a percentage is about 40%. In some embodiments, a percentage is about 45%. In some embodiments, a percentage is about 50%. In some embodiments, a percentage is about 55%. In some embodiments, a percentage is about 60%. In some embodiments, a percentage is about 65%. In some embodiments, a percentage is about 70%. In some embodiments, a percentage is about 75%. In some embodiments, a percentage is about 80%. In some embodiments, a percentage is about 85%. In some embodiments, a percentage is about 90%.

In some embodiments, one or more (e.g., 1-20, 1, 2, 3, 4, 5, 6, 7, 8, 9, or 10, etc.) mismatches exist in a second domain when an oligonucleotide is aligned with a target nucleic acid for complementarity. In some embodiments, there is 1 mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches.

In some embodiments, one or more (e.g., 1-20, 1, 2, 3, 4, 5, 6, 7, 8, 9, or 10, etc.) wobbles exist in a second domain when an oligonucleotide is aligned with a target nucleic acid for complementarity. In some embodiments, there is 1 wobble. In some embodiments, there are 2 wobbles. In some embodiments, there are 3 wobbles. In some embodiments, there are 4 wobbles. In some embodiments, there are 5 wobbles. In some embodiments, there are 6 wobbles. In some embodiments, there are 7 wobbles. In some embodiments, there are 8 wobbles. In some embodiments, there are 9 wobbles. In some embodiments, there are 10 wobbles.

In some embodiments, duplexes of oligonucleotides and target nucleic acids in a second domain region comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3- 6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, a second domain is fully complementary to a target nucleic acid.

In some embodiments, a second domain comprises one or more modified nucleobases.

In some embodiments, a second domain comprise a nucleoside opposite to a target adenosine, e.g., when the oligonucleotide forms a duplex with a target nucleic acid. In some embodiments, an opposite nucleobase is optionally substituted or protected U, or is an optionally substituted or protected tautomer of U. In some embodiments, an opposite nucleobase is U.

In some embodiments, an opposite nucleobase has weaker hydrogen bonding with a target adenine of a target adenosine compared to U. In some embodiments, an opposite nucleobase forms fewer hydrogen bonds with a target adenine of a target adenosine compared to U. In some embodiments, an opposite nucleobase forms one or more hydrogen bonds with one or more amino acid residues of a protein, e.g., ADAR, which residues form one or more hydrogen bonds with U opposite to a target adenosine. In some embodiments, an opposite nucleobase forms one or more hydrogen bonds with each amino acid residue of ADAR that forms one or more hydrogen bonds with U opposite to a target adenosine. In some embodiments, by weakening hydrogen boding with a target A and/or maintaining or enhancing interactions with proteins such as ADAR1, ADAR2, etc., certain opposite nucleobase facilitate and/or promote adenosine modification, e.g., by ADAR proteins such as ADAR1 and ADAR2.

In some embodiments, an opposite nucleobase is optionally substituted or protected C, or is an optionally substituted or protected tautomer of C. In some embodiments, an opposite nucleobase is C. In some embodiments, an opposite nucleobase is optionally substituted or protected A, or is an optionally substituted or protected tautomer of A. In some embodiments, an opposite nucleobase is A. In some embodiments, an opposite nucleobase is optionally substituted or protected nucleobase of pseudoisocytosine, or is an optionally substituted or protected tautomer of the nucleobase of pseudoisocytosine. In some embodiments, an opposite nucleobase is the nucleobase of pseudoisocytosine.

In some embodiments, a nucleoside, e.g., a nucleoside opposite to abasic as described herein (e.g., having the structure of L010, L012, L028, etc.).

Many useful embodiments of modified nucleobases, e.g., for opposite nucleobases, are also described below. In some embodiments, as described herein (e.g., in various oligonucleotides), the present disclosure provides oligonucleotides comprising a nucleobase, e.g., of a nucleoside opposite to a target nucleoside such as A, which is or comprises C, A, aC, b007U, b001U, b001A, b002U, b001C, b003U, b002C, b004U, b003C, b005U, b0021, b006U, b0031, b008U, b009U, b002A, b003A, b001G, or zdnp. In some embodiments, a nucleobase is C. In some embodiments, a nucleobase is A. In some embodiments, a nucleobase is aC. In some embodiments, a nucleobase is b007U. In some embodiments, a nucleobase is b001U. In some embodiments, a nucleobase is b001A. In some embodiments, a nucleobase is b002U. In some embodiments, a nucleobase is b001C. In some embodiments, a nucleobase is b003U. In some embodiments, a nucleobase is b002C. In some embodiments, a nucleobase is b004U. In some embodiments, a nucleobase is b003C. In some embodiments, a nucleobase is b005U. In some embodiments, a nucleobase is b0021. In some embodiments, a nucleobase is b006U. In some embodiments, a nucleobase is b0031. In some embodiments, a nucleobase is b008U. In some embodiments, a nucleobase is b009U. In some embodiments, a nucleobase is b002A. In some embodiments, a nucleobase is b003A. In some embodiments, a nucleobase is b001G. In some embodiments, a nucleobase is or zdnp. In some embodiments, as those skilled in the art appreciate, a nucleobase is protected, e.g., for oligonucleotide synthesis. For example, in some embodiments, a nucleobase is protected b001A having the structure of

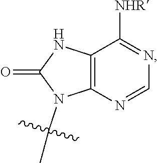

wherein R′ is as described herein. In some embodiments, R′ is —C(O)R. In some embodiments, R′ is —C(O)Ph.

Certain Modified Nucleobases

In some embodiments, BA is or comprises Ring BA or a tautomer thereof, wherein Ring BA is an optionally substituted, 5-20 membered, monocyclic, bicyclic or polycyclic ring having 0-10 heteroatoms. In some embodiments, Ring BA is or comprises an optionally substituted, 5-20 membered, monocyclic, bicyclic or polycyclic having 1-10 heteroatoms, wherein at least one heteroatom is nitrogen. In some embodiments, Ring BA is saturated. In some embodiments, Ring BA comprises one or more unsaturation. In some embodiments, Ring BA is partially unsaturated. In some embodiments, Ring BA is aromatic.

In some embodiments, BA is or comprises Ring BA, wherein Ring BA is an optionally substituted, 5-20 membered, monocyclic, bicyclic or polycyclic ring having 0-10 heteroatoms. In some embodiments, Ring BA is or comprises an optionally substituted, 5-20 membered, monocyclic, bicyclic or polycyclic having 1-10 heteroatoms, wherein at least one heteroatom is nitrogen. In some embodiments, Ring BA is saturated. In some embodiments, Ring BA comprises one or more unsaturation. In some embodiments, Ring BA is partially unsaturated. In some embodiments, Ring BA is aromatic.

In some embodiments, BA is or comprises Ring BA. In some embodiments, BA is Ring BA. In some embodiments, BA is or comprises a tautomer of Ring BA. In some embodiments, BA is a tautomer of Ring BA.

In some embodiments, structures of the present disclosure contain one or more optionally substituted rings (e.g., Ring BA, -Cy-, Ring BA^{A}, R, formed by R groups taken together, etc.). In some embodiments, a ring is an optionally substituted C₃₋₃₀, C₃₋₂₀, C₃₋₁₅, C₃₋₁₀, C₃₋₉, C₃₋₈, C₃₋₇, C₃₋₆, C₅₋₅₀, C₅₋₂₀, C₅₋₁₅, C₅₋₁₀, C₅₋₉, C₅₋₈, C₅₋₇, C₅₋₆, or 3-30 (e.g., 3-30, 3-20, 3-15, 3-10, 3-9, 3-8, 3-7, 3-6, 5-50, 5-20, 5-15, 5-10, 5-9, 5-8, 5-7, 5-6, or 1, 2, 3, 4, 5, 6, 7, 8, 9, 10, 11, 12, 13, 14, 15, 16, 17, 18, 19, 20, 25, 30, etc.) membered monocyclic, bicyclic or polycyclic ring having 0-10 (e.g., 1-10, 1-5, 0, 1, 2, 3, 4, 5, 6, 7, 8, 9, 10, etc.) heteroatoms. In some embodiments, a ring is an optionally substituted 3-10 membered monocyclic or bicyclic, saturated, partially saturated or aromatic ring having 0-3 heteroatoms. In some embodiments, a ring is substituted. In some embodiments, a ring is not substituted. In some embodiments, a ring is 3, 4, 5, 6, 7, 8, 9, or 10 membered. In some embodiments, a ring is 5, 6, or 7-membered. In some embodiments, a ring is 5-membered. In some embodiments, a ring is 6-membered. In some embodiments, a ring is 7-membered. In some embodiments, a ring is monocyclic. In some embodiments, a ring is bicyclic. In some embodiments, a ring is polycyclic. In some embodiments, a ring is saturated. In some embodiments, a ring contains at least one unsaturation. In some embodiments, a ring is partially unsaturated. In some embodiments, a ring is aromatic. In some embodiments, a ring has 0-5 heteroatoms. In some embodiments, a ring has 1-5 heteroatoms. In some embodiments, a ring has one or more heteroatoms. In some embodiments, a ring has 1 heteroatom. In some embodiments, a ring has 2 heteroatoms. In some embodiments, a ring has 3 heteroatoms. In some embodiments, a ring has 4 heteroatoms. In some embodiments, a ring has 5 heteroatoms. In some embodiments, a heteroatom is nitrogen. In some embodiments, a heteroatom is oxygen. In some embodiments, a ring is substituted, e.g., substituted with one or more alkyl groups and optionally one or more other substituents as described herein. In some embodiments, a substituent is methyl.

In some embodiments, each monocyclic ring unit of a monocyclic, bicyclic, or polycyclic ring of the present disclosure (e.g., Ring BA, —Cy-, Ring BA^{A}, R, formed by R groups taken together, etc.) is independently an optionally substituted 5-7 membered, saturated, partially unsaturated or aromatic ring having 0-5 heteroatoms. In some embodiments, one or more monocyclic units independently comprise one or more unsaturation. In some embodiments, one or more monocyclic units are saturated. In some embodiments, one or more monocyclic units are partially saturated. In some embodiments, one or more monocyclic units are aromatic. In some embodiments, one or more monocyclic units independently have 1-5 heteroatoms. In some embodiments, one or more monocyclic units independently have at least one nitrogen atom. In some embodiments, each monocyclic unit is independently 5- or 6-membered. In some embodiments, a monocyclic unit is 5-membered. In some embodiments, a monocyclic unit is 5-membered and has 1-2 nitrogen atom. In some embodiments, a monocyclic unit is 6-membered. In some embodiments, a monocyclic unit is 6-membered and has 1-2 nitrogen atom. Rings and monocyclic units thereof are optionally substituted unless otherwise specified.

Without the intention to be limited by any particular theory, the present disclosure recognizes that in some embodiment, structures of nucleobases (e.g. BA) can impact interactions with proteins (e.g., ADAR proteins such as ADAR1, ADAR2, etc.). In some embodiments, provided oligonucleotides comprise nucleobases that can facility interaction of an oligonucleotide with an enzyme, e.g., ADAR1. In some embodiments, provided oligonucleotides comprise nucleobases that may reduce strength of base pairing (e.g., compared to A-T/U or C-G). In some embodiments, the present disclosure recognizes that by maintaining and/or enhancing interactions (e.g., hydrogen bonding) of a first nucleobase with a protein (e.g., an enzyme like ADAR1) and/or reducing interactions (e.g., hydrogen bonding) of a first nucleobase with its corresponding nucleobase (e.g., A) on the other strand in a duplex, modification of the corresponding nucleobase by a protein (e.g., an enzyme like ADAR1) can be significantly improved. In some embodiments, the present disclosure provides oligonucleotides comprises such a first nucleobase (e.g., various embodiments of BA described herein). Exemplary embodiments of such as a first nucleobase are as described herein. In some embodiments, when an oligonucleotide comprising such a first nucleobase is aligned with another nucleic acid for maximum complementarity, the first nucleobase is opposite to A. In some embodiments, such an A opposite to the first nucleobase, as exemplified in many embodiments of the present disclosure, can be efficiently modified using technologies of the present disclosure.

In some embodiments, Ring BA comprises a moiety [symbol] X² [symbol] X³ [symbol] , wherein each variable is independently as described herein. In some embodiments, Ring BA comprises a moiety [symbol] X² [symbol] X³ [symbol] X⁴ [symbol] , wherein each variable is independently as described herein. In some embodiments, Ring BA comprises a moiety —X¹( [symbol] ) [symbol] X² [symbol] X³ [symbol] , wherein each variable is independently as described herein. In some embodiments, Ring BA comprises a moiety —X¹( [symbol] ) [symbol] X² [symbol] X³ [symbol] X⁴ [symbol] , wherein each variable is independently as described herein. In some embodiments, X¹ is bonded to a sugar. In some embodiments, X¹ is —N(−)-. In some embodiments, X¹ is —C(═)-. In some embodiments, X² is —C(O)—. In some embodiments, X³ is —NH—. In some embodiments, X⁴ is not —C(O)—. In some embodiments, X⁴ is —C(O)—, and forms an intramolecular hydrogen bond, e.g., with a moiety of the same nucleotidic unit (e.g., within the same BA unit (e.g., with a hydrogen bond donor (e.g., —OH, SH, etc.) of X⁵). In some embodiments, X⁴ is —C(═NH)—. In some embodiments, Ring BA comprises a moiety [symbol] X^{4′} [symbol] X^{5′} [symbol] , wherein each variable is independently as described herein. In some embodiments, X^{4′} is —C(O)—. In some embodiments, X^{5′} is —NH—.

In some embodiments, BA is optionally substituted or protected C or a tautomer thereof. In some embodiments, BA is optionally substituted or optionally protected C. In some embodiments, BA is an optionally substituted or optionally protected tautomer of C. In some embodiments, BA is C. In some embodiments, BA is substituted C. In some embodiments, BA is protected C. In some embodiments, BA is an substituted tautomer of C. In some embodiments, BA is an protected tautomer of C.

In some embodiments, Ring BA has the structure of formula BA-I:

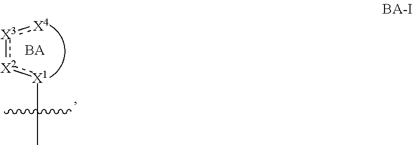

wherein:

Ring BA is an optionally substituted, 5-20 membered, monocyclic, bicyclic or polycyclic, saturated, partially saturated or aromatic ring having 1-10 heteroatoms;

each [symbol] is independent a single or double bond;

X¹ is —N(−)- or —C(−)=;

X² is —C(O)—, —C(R^{B2})═, or C(OR^{B2})═, wherein R^{B2} is -L^{B2}-R′;

X³ is —N(R^{B3})— or —N═, wherein R^{B3} is -L^{B3}-R′;

X⁴ is —C(R^{B4}), —C(—N(R^{B4})₂)═, —C(R^{B4})₂—, —C(O)—, or —C(═NR^{B4})—, wherein each R^{B4} is independently -L^{B4}R^{B41}, or two R^{B4} on the same atom are taken together to form ═O, ═C(-L^{B4}-R^{B41})₂, ═N-L^{B4}-R^{B41}, or optionally substituted ═CH₂ or ═NH, wherein each R^{B41} is independently R′;

each of L^{B2}, L^{B3}, and L^{B4} is independently L^{B};

each L^{B} is independently a covalent bond, or an optionally substituted bivalent C₁₋₁₀ saturated or partially unsaturated chain having 0-6 heteroatoms, wherein one or more methylene unit is optionally and independently replaced with -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;

each -Cy- is independently an optionally substituted, 3-20 membered, monocyclic, bicyclic or polycyclic ring having 0-10 heteroatoms;

each R′ is independently —R, —C(O)R, —C(O)OR, —C(O)N(R)₂, or —SO₂R; and

each R is independently —H, or an optionally substituted group selected from C1-20 aliphatic, C₁₋₂₀ heteroaliphatic having 1-10 heteroatoms, C₆₋₂₀ aryl, C₆₋₂₀ arylaliphatic, C₆₋₂₀ arylheteroaliphatic having 1-10 heteroatoms, 5-20 membered heteroaryl having 1-10 heteroatoms, and 3-20 membered heterocyclyl having 1-10 heteroatoms, or:

two R groups are optionally and independently taken together to form a covalent bond, or:

two or more R groups on the same atom are optionally and independently taken together with the atom to form an optionally substituted, 3-20 membered, monocyclic, bicyclic or polycyclic ring having, in addition to the atom, 0-10 heteroatoms; or:

two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, Ring BA (e.g., one of formula BA-I) has the structure of formula BA-I-a:

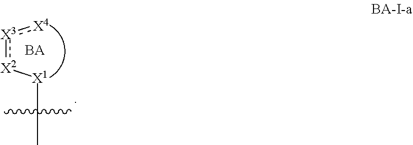

In some embodiments, Ring BA (e.g., one of formula BA-I, BA-I-a, etc.) has the structure of formula BA-I-b:

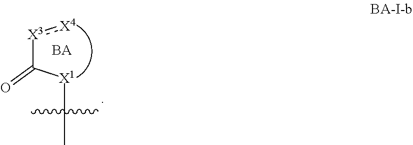

In some embodiments, Ring BA (e.g., one of formula BA-I) has the structure of formula BA-II:

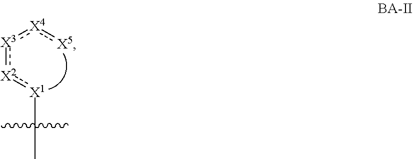

wherein:

X⁵ is —C(R^{B5})₂—, —N(R^{B5}), C(R^{B5})═, —C(O)—, or —N═, wherein each R^{B5} is independently halogen, or -L^{B5}-R^{B51}, wherein R^{B51} is —R′, —N(R′)₂, —OR′, or —SR′;

L^{B5} is L^{B}; and

each other variable is independently as described herein.

In some embodiments, Ring BA (e.g., one of formula BA-I, BA-I-a, BA-II, etc.) has the structure of formula BA-II-a:

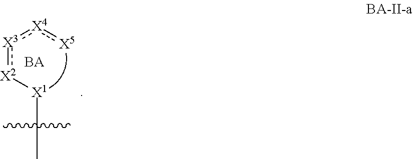

In some embodiments, Ring BA (e.g., one of formula BA-I, BA-I-a, BA-I-b, BA-II, BA-II-a, etc.) has the structure of formula BA-II-b:

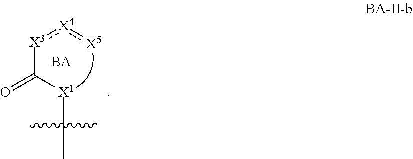

In some embodiments, Ring BA (e.g., one of formula BA-I, BA-II, etc.) has the structure of formula BA-III:

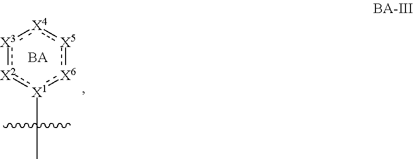

wherein:

X⁶ is —C(R^{B6})═, —C(OR^{B6})═, —C(R^{B6})₂—, —C(O)— or —N═, wherein each R^{B6} is independently -L^{B6}-R^{B61}, or two R^{B6} on the same atom are taken together to form ═O, ═C(-L^{B6}-R^{B61})₂, ═N-L^{B6}R^{B61}, or optionally substituted ═CH₂ or ═NH, wherein each R^{B61} is independently R′;

L^{B6} is L^{B}; and

each other variable is independently as described herein.

In some embodiments, Ring BA (e.g., one of formula BA-I, BA-I-a, BA-II, BA-II-a, BA-III, etc.) has the structure of formula BA-III-a:

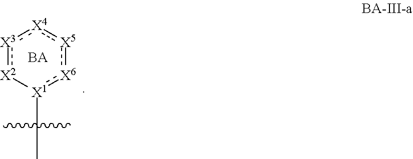

In some embodiments, Ring BA (e.g., one of formula BA-I, BA-I-a, BA-I-b, BA-II, BA-II-a, BA-II-b, BA-III, BA-III-a, etc.) has the structure of formula BA-III-b:

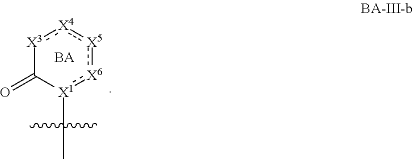

In some embodiments, Ring BA (e.g., one of formula BA-I, BA-II, etc.) has the structure of formula BA-IV:

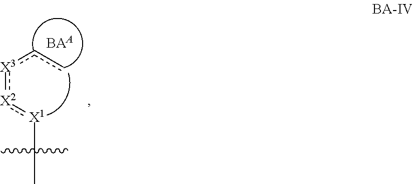

wherein:

Ring BA^{A} is an optionally substituted 5-14 membered, monocyclic, bicyclic or polycyclic ring having 0-5 heteroatoms, and

each other variable is independently as described herein.

In some embodiments, Ring BA (e.g., one of formula BA-I, BA-I-a, BA-II, BA-II-a, etc.) has the structure of formula BA-IV-a:

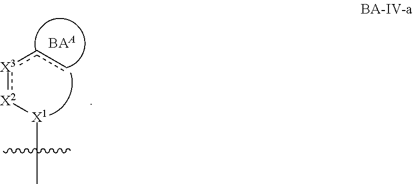

In some embodiments, Ring BA (e.g., one of formula BA-I, BA-I-a, BA-II, BA-II-a, etc.) has the structure of formula BA-IV-b:

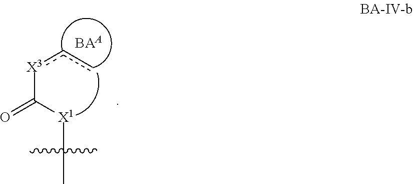

In some embodiments, Ring BA (e.g., one of formula BA-I, BA-II, BA-III, BA-IV, etc.) has the structure of formula BA-V:

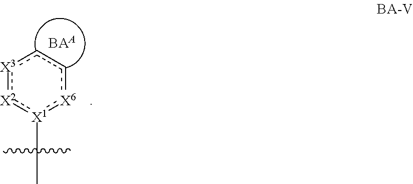

In some embodiments, Ring BA (e.g., one of formula BA-I, BA-I-a, BA-II, BA-II-a, BA-III, BA-III-a, BA-IV, BA-IV-a, BA-V, etc.) has the structure of formula BA-V-a:

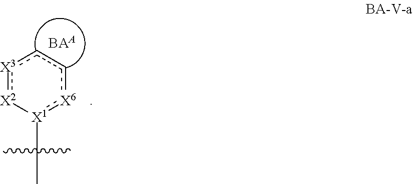

In some embodiments, Ring BA (e.g., one of formula BA-I, BA-I-a, BA-I-b, BA-II, BA-II-a, BA-II-b, BA-III, BA-III-a, BA-III-b, BA-IV, BA-IV-a, BA-IV-b, BA-V, BA-V-a, etc.) has the structure of formula BA-V-a:

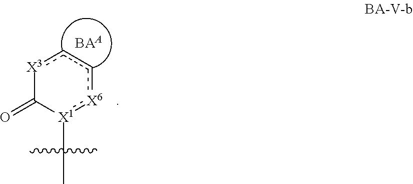

In some embodiments, Ring BA has the structure of formula BA-VI:

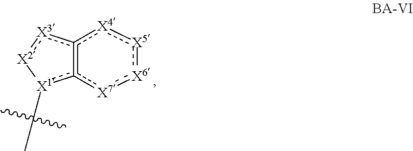

wherein:

X^{1′} is —N(−)- or —C(−)=;

X^{2′} is —C(O)— or —C(R^{B2′})═, wherein R^{B2′}—R′;

each [symbol] is independent a single or double bond;

X^{3′} is —N(R^{B3′})— or —N═, wherein R^{B3′} is R′;

X^{4′} is —C(R^{B4′})═, C(OR^{B4′})═, —C(—N(R^{B4′})₂)═, —C(R^{B4′})₂—, —C(O)—, or —C(═N^{RB4′}), wherein each R^{B4′} is independently -L^{B4′}-R^{B41′}, or two R^{B4′} on the same atom are taken together to form ═O, ═C(-L^{B4′}-R^{B41′})₂, ═N-L^{B4′}-R^{B41′}, or optionally substituted ═CH₂ or ═NH, wherein each R^{B41′} is independently —R′;

X^{5′} is —N(R^{B5}) or —N═, wherein R^{B5′} is R′;

X^{6′} is —C(R^{B6′})═, —C(OR^{B6′})═, —C(R^{B6′})₂—, —C(O)— or —N═, wherein each R^{B6′} is independently -L^{B6′}-R^{B61′}, or two R^{B6′} on the same atom are taken together to form ═O, ═C(-L^{B6′}-R^{B61′})₂, ═N-L^{B6′}-R^{B61′}, or optionally substituted ═CH₂ or ═NH, wherein each R^{B61′} is independently R′;

X^{7′} is —C(R^{B7′}), —C(OR^{B6′}), —C(R^{B7′})₂—, —C(O)—, —N(R^{B7′})—, or —N═, wherein each R^{B7′} is independently -L^{7′}-R^{B71′}, or two R^{B7′} on the same atom are taken together to form ═O, ═C(-L^{7′}-R^{B71′})₂, ═N-L^{7′}-R^{B71′}, or optionally substituted ═CH₂ or ═NH, wherein each R^{B71′} is independently R′;

each of L^{B2′}, L^{B3′}, L^{B4′}, L^{B5′} and L^{B6′} is independently L^{B}; and

each other variable is independently as described herein.

In some embodiments, [symbol] is a single bond. In some embodiments, [symbol] is a double bond.

In some embodiments, X¹ is —(N—)—. In some embodiments, X¹ is —C(−)=.

In some embodiments' X² is —C(O)—. In some embodiments, X² is —C(R^{B2})═. In some embodiments, X² is —C(OR^{B2})═. In some embodiments, X² is —CH═.

In some embodiments, L^{B2} is a covalent bond.

In some embodiments, R^{B2} is a protecting group, e.g., a hydroxyl protecting group suitable for oligonucleotide synthesis. In some embodiments, R^{B2} is R′. In some embodiments, R^{B2} is —H.

In some embodiments, X³ is —N(R^{B3})—. In some embodiments, X³ is —NH—. In some embodiments, X³ is —N═.

In some embodiments, L^{B3} is a covalent bond.

In some embodiments, R^{B3} is a protecting group, e.g., an amino protecting group suitable for oligonucleotide synthesis (e.g., Bz). In some embodiments, R^{B3} is R′. In some embodiments, R^{B3} is —C(O)R. In some embodiments, R^{B3} is R. In some embodiments, R^{B3} is —H.

In some embodiments, X⁴ is —C(R^{B4})═. In some embodiments, X⁴ is —C(R)═. In some embodiments, X⁴ is —CH═. In some embodiments, X⁴ is —C(OR^{B4})═. In some embodiments, X⁴ is —C(—N(R^{B4})₂)═. In some embodiments, X⁴ is —C(—NHR^{B4})═. In some embodiments, X⁴ is —C(—NHR′)═. In some embodiments, X⁴ is —C(—NHR′)═. In some embodiments, X⁴ is —C(—NH₂)═. In some embodiments, X⁴ is —C(—NHC(O)R)═. In some embodiments, X⁴ is —C(R^{B4})₂—. In some embodiments, X⁴ is —CH₂—. In some embodiments, X⁴ is —C(O)—. In some embodiments, X⁴ is —C(O)—, wherein O forms a intramolecular hydrogen bond. In some embodiments, O forms a hydrogen bond with a hydrogen bond donor of X⁵ of the same BA. In some embodiments, X⁴ is —C(═NR^{B4})—. In some embodiments, X⁴ is —C((═NR^{B4})—, wherein N forms a intramolecular hydrogen bond. In some embodiments, N forms a hydrogen bond with a hydrogen bond donor of X⁵ of the same BA.

In some embodiments, R^{B4}-L^{B4}-R^{B41}. In some embodiments, two R^{B4} on the same atom are taken together to form ═O, ═C(-L^{B4}-R^{B41})₂, ═N-L^{B4}-R^{B41}, or optionally substituted ═CH₂ or ═NH.

In some embodiments, two R^{B4} on the same atom are taken together to form ═O. In some embodiments, two R^{B4} on the same atom are taken together to form ═C(-L^{B4}-R^{B41})₂. In some embodiments, ═C(-L^{B4}-R^{B41})₂ is ═CH-L^{B4}-R^{B41}. In some embodiments, ═C(-L^{B4}-R^{B41})₂ is ═CHR′. In some embodiments, ═C(-L^{B4}-R^{B41})₂ is ═CHR. In some embodiments, two R^{B4} on the same atom are taken together to form ═N-L^{B4}-R^{B41}. In some embodiments, ═N-L^{B4}-R^{B41} is ═N—R. In some embodiments, two R^{B4} on the same atom are taken together to form ═CH₂. In some embodiments, two R^{B4} on the same atom are taken together to form ═NH. In some embodiments, a formed group is a suitable protecting group, e.g., amino protecting group, for oligonucleotide synthesis.

In some embodiments, X⁴ is —C(—N═C(-L^{B4}-R^{B41})₂)═. In some embodiments, X⁴ is —C(—N═CH-L^{B4}-R^{B41})═. In some embodiments, X⁴ is —C(—N═CH—N(CH₃)₂)═.

In some embodiments, R of X⁴ (e.g., of —C(═N—R)—, ═C(R)—, etc.) are optionally taken together with another R, e.g., of X⁵, to form a ring as described herein.

In some embodiments, R^{B4} is R′. In some embodiments, R^{B4} is R. In some embodiments, R^{B4} is —H.

In some embodiments, R^{B4} is a protecting group, e.g., an amino or hydroxyl protecting group suitable for oligonucleotide synthesis. In some embodiments, R^{B4} is R′. In some embodiments, R^{B4} is —CH₂CH₂-(4-nitrophenyl).

In some embodiments, L^{B4} is a covalent bond. In some embodiments, L^{B4} is not a covalent bond. In some embodiments, at least one methylene unit is replaced with —C(O)—. In some embodiments, at least one methylene unit is replaced with —C(O)N(R′)—. In some embodiments, at least one methylene unit is replaced with —N(R′)—. In some embodiments, at least one methylene unit is replaced with —NH—. In some embodiments, L^{B4} is or comprises optionally substituted —N═CH—.

In some embodiments, R^{B41} is R′. In some embodiments, R^{B41} is —H. In some embodiments, R^{B41} is R. In some embodiments, R is optionally substituted phenyl. In some embodiments, R is phenyl.

In some embodiments, X⁵ is —C(R^{B5})₂—. In some embodiments, X⁵ is -ChR^{B5}—. In some embodiments, X⁵ is —CH₂—. In some embodiments, X⁵ is —N(R^{B5}). In some embodiments, X⁵ is —NH—. In some embodiments, X⁵ is —C(R^{B5})═. In some embodiments, X⁵ is —C(R)═. In some embodiments, X⁵ is —CH═. In some embodiments, X⁵ is —N═. In some embodiments, X⁵ is —C(O)—.

In some embodiments, R^{B5} is halogen. In some embodiments, R^{B5} is L^{B}5-R^{B51}. In some embodiments, R^{B5} is -L^{B5}-R^{B51}, wherein R^{B51} is R′, —NHR′, —OH, or —SH. In some embodiments, R^{B5} is -L^{B5}-R^{B51}, wherein R^{B51} is —NHR, —OH, or —SH. In some embodiments, R^{B5} is -L^{B5}-R^{B51}, wherein R^{B51} is —NH₂, —OH, or —SH. In some embodiments, R^{B5} is —C(O)—R^{B51}. In some embodiments, R^{B5} is R′. In some embodiments, R^{B5} is R. In some embodiments, R^{B5} is —H. In some embodiments, R^{B5} is —OH. In some embodiments, R^{B5} is —CH₂OH.

In some embodiments, when X⁴ is —C(O)—, X⁵ is —C(R^{B5})₂—, —C(R^{B5}), or —N(R^{B5})—, wherein R^{B5} is -L^{B5}-R^{B51}, wherein R^{B51} is —NHR′, —OH, or —SH. In some embodiments, X⁴ is —C(O)—, and R^{B51} is or comprises a hydrogen bond donor, which forms a hydrogen bond with the O of X⁴.

In some embodiments, L^{B5} is a covalent bond. In some embodiments, L^{B5} is or comprises —C(O)—. In some embodiments, L^{B5} is or comprises —O—. In some embodiments, L^{B5} is or comprises —OC(O)—. In some embodiments, L^{B5} is or comprises —CH₂OC(O)—.

In some embodiments, R⁵¹ is —R′. In some embodiments, R⁵¹ is —R. In some embodiments, R⁵¹ is —H. In some embodiments, R⁵¹ is —N(R′)₂. In some embodiments, R⁵¹ is —NHR′. In some embodiments, R⁵¹ is —NHR. In some embodiments, R⁵¹ is —NH₂. In some embodiments, R⁵¹ is —OR′. In some embodiments, R⁵¹ is —OR. In some embodiments, R⁵¹ is —OH. In some embodiments, R⁵¹ is —SR′. In some embodiments, R⁵¹ is —SR. In some embodiments, R⁵¹ is —SH. In some embodiments, R is benzyl. In some embodiments, R is optionally substituted phenyl. In some embodiments, R is phenyl. In some embodiments, R is methyl.

In some embodiments, R^{B5} is —C(O)—R^{B51}. In some embodiments, R^{B5} is —C(O)NHCH₂Ph. In some embodiments, R^{B5} is —C(O)NHPh. In some embodiments, R^{B5} is —C(O)NHCH₃. In some embodiments, R^{B5} is —OC(O)—R^{B51}. In some embodiments, R^{B5} is —OC(O)—R. In some embodiments, R^{B5} is —OC(O)CH₃.

In some embodiments, X⁵ is directly bonded to X¹, and Ring BA is 5-membered.

In some embodiments, X⁶ is —C(R^{B6})═. In some embodiments, X⁶ is —CH═. In some embodiments, X⁶ is —C(OR^{B6})═. In some embodiments, X⁶ is —C(R^{B6})₂—. In some embodiments, X⁶ is —CH₂—. In some embodiments, X⁶ is —C(O)—. In some embodiments, X⁶ is —N═.

In some embodiments, R^{B6} is -L^{B6}-R^{B61}. In some embodiments, two R^{B6} on the same atom are taken together to form ═O, ═C(-L^{B6}-R^{B61})₂, ═N-L^{B6}R^{B61}, or optionally substituted ═CH₂ or ═NH. In some embodiments, two R^{B6} on the same atom are taken together to form ═O. In some embodiments, L^{B6} is a covalent bond. In some embodiments, R^{B6} is R. In some embodiments, R^{B6} is —H.

In some embodiments, R^{B6} is a protecting group, e.g., an amino or hydroxyl protecting group suitable for oligonucleotide synthesis. In some embodiments, R^{B6} is R. In some embodiments,

In some embodiments, L^{B6} is a covalent bond. In some embodiments, L^{B6} is optionally substituted C₁₋₁₀ alkylene. In some embodiments, L^{B6} is —CH₂CH₂—. In some embodiments, R^{B6} is —CH₂CH₂-(4-nitrophenyl).

In some embodiments, R^{B61} is R′. In some embodiments, R^{B61} is R. In some embodiments, R^{B61} is —H.

In some embodiments, Ring BA^{A} is 5-membered. In some embodiments, Ring BA^{A} is 5-membered. In some embodiments, Ring BA^{A} has one heteroatom. In some embodiments, Ring BA^{A} has 2 heteroatoms. In some embodiments, a heteroatom is nitrogen. In some embodiments, a heteroatom is oxygen.

In some embodiments, X^{1′} is —(N—)-. In some embodiments, X^{1′} is —C(−)=.

In some embodiments, X^{2′} is —C(O)—. In some embodiments, X^{2′} is —C(R^{B2′})═. In some embodiments, X^{2′} is —CH═.

In some embodiments, L^{B2′} is a covalent bond.

In some embodiments, R^{B2′} is R′. In some embodiments, R^{B2′} is R. In some embodiments, R^{B2′} is —H. In some embodiments, XT is —CH═.

In some embodiments, X^{3′} is —N(R^{B3′})—. In some embodiments, X^{3′} is —N(R′)—. In some embodiments, X^{3′} is —NH—. In some embodiments, X^{3′} is —N═.

In some embodiments, L^{B3} is a covalent bond.

In some embodiments, R^{B3′} is R′. In some embodiments, R^{B3′} is R. In some embodiments, R^{B3} is —H.

In some embodiments, X^{4′} is —C(R^{B4′})═. In some embodiments, X⁴ is —C(OR^{B4′})═. In some embodiments, X⁴ is —C(—N(R^{B4′})₂)═. In some embodiments, X^{4′} is —C(—NHR^{B4′})═. In some embodiments, X^{4′} is —C(—NH₂)═. In some embodiments, X^{4′} is —C(—NHR′)═. In some embodiments, X^{4′} is —C(—NHC(O)R)═. In some embodiments, X^{4′} is —C(R^{B4′})₂—. In some embodiments, X^{4′} is —C(O)—. In some embodiments, X⁴ is —C(═NR^{B4})—.

In some embodiments, R^{B4′} is -L^{B4′}R^{B41′}. In some embodiments, two R^{B4′} on the same atom are taken together to form ═O, ═C(-L^{B4′}-R^{B41′})₂, ═N-L^{B4′}-R^{B41′}, or optionally substituted ═CH₂ or ═NH. In some embodiments, two R^{B4′} on the same atom are taken together to form ═O. In some embodiments, two R^{B4′} on the same atom are taken together to form ═C(-L^{B4′}-R^{B41′})₂. In some embodiments, two R^{B4′} on the same atom are taken together to form ═N-L^{B4′}-R^{B41′}. In some embodiments, two R^{B4′} on the same atom are taken together to form ═CH₂. In some embodiments, two R^{B4′} on the same atom are taken together to form ═NH. In some embodiments, a formed group is a suitable protecting group, e.g., amino protecting group, for oligonucleotide synthesis.

In some embodiments, X^{4′} is —C(—N═C(-L^{B4′}-R^{B41′})₂)═. In some embodiments, X^{4′} is —C(—N═CH-L^{B4′}-R^{B41′})═. In some embodiments, X^{4′} is —C(—N═CH—N(CH₃)₂)═.

In some embodiments, R^{B4′} is R′. In some embodiments, R^{B4′} is R. In some embodiments, R^{B4} is —H.

In some embodiments, R^{B4′} is a protecting group, e.g., an amino or hydroxyl protecting group suitable for oligonucleotide synthesis. In some embodiments, R^{B4′} is R′. In some embodiments, R^{B4′} is —CH₂CH₂-(4-nitrophenyl).

In some embodiments, L^{B4′} is a covalent bond. In some embodiments, L^{B4′} is optionally substituted C₁₋₁₀ alkylene. In some embodiments, L^{B4′} is —CH₂CH₂—. In some embodiments, at least one methylene unit is replaced with —N(R′)—. In some embodiments, R′ is R. In some embodiments, R is optionally substituted phenyl. In some embodiments, R is phenyl. In some embodiments, R is methyl. In some embodiments, R is —H.

In some embodiments, R^{B41′} is R′. In some embodiments, R^{B41′} is R. In some embodiments, R^{B41′} is —H.

In some embodiments, X^{5′} is —N(R^{B5′}). In some embodiments, X^{5′} is —NH—. In some embodiments, X^{5′} is —N═.

In some embodiments, L^{B5′} is a covalent bond.

In some embodiments, R^{B5′} is R′. In some embodiments, R^{B5′} is R. In some embodiments, R^{B5′} is —H.

In some embodiments, X^{6′} is —C(R^{B6′})═. In some embodiments, X^{6′} is —CH═. In some embodiments, X^{6′} is —C(OR^{B6′})═. In some embodiments, X^{6′} is —C(R^{B6′})₂—. In some embodiments, X^{6′} is —C(O)—. In some embodiments, X^{6′} is —N═.

In some embodiments, R^{B6}, is -L^{B6′}-R^{B61′}. In some embodiments, two R^{B6′} on the same atom are taken together to form ═O, ═C(-L^{B6′}-R^{B61′})₂, ═N-L^{B6}-R^{B61′}, or optionally substituted CH₂ or ═NH. In some embodiments, two R^{B6′} on the same atom are taken together to form ═O.

In some embodiments, L^{B6′} is a covalent bond. In some embodiments, L^{B6′} is optionally substituted C₁₋₁₀ alkylene. In some embodiments, L^{B6′} is —CH₂CH₂—.

In some embodiments, R^{B6′} is R′. In some embodiments, R^{B6′} is R. In some embodiments, R^{B6} is —H. In some embodiments, R^{B6}, is a protecting group, e.g., an amino or hydroxyl protecting group suitable for oligonucleotide synthesis. In some embodiments, R^{B6′} is R′. In some embodiments, R^{B6′} is —CH₂CH₂-(4-nitrophenyl).

In some embodiments, R^{B61′} is R′. In some embodiments, R^{B61′} is R. In some embodiments, R^{B61} is —H.

In some embodiments, X⁷ is —C(R^{B7′})═. In some embodiments, X^{7′} is —CH═. In some embodiments, X^{7′} is —C(OR^{B7′})═. In some embodiments, X^{7′} is —C(R^{B7′})₂—. In some embodiments, X^{7′} is —C(O)—. In some embodiments, X^{7′} is —N(R^{B7})—. In some embodiments, X^{7′} is —NH—. In some embodiments, X^{7′} is —N═.

In some embodiments, R^{B7} is -L^{7′}-R^{B71′}. In some embodiments, two R^{B7′} on the same atom are taken together to form ═O, ═C(-L-R^{B71′})₂, ═N-L⁷-R^{B71}, or optionally substituted ═CH₂ or ═NH. In some embodiments, two R^{B7′} on the same atom are taken together to form ═O. In some embodiments, L^{7′} is a covalent bond. In some embodiments, R^{B7′} is R. In some embodiments, R^{B7′} is —H.

In some embodiments, R^{B71′} is R′. In some embodiments, R^{B71′} is R. In some embodiments, R^{B71′} is —H.

In some embodiments, L^{B} is a covalent bond. In some embodiments, L^{B} is an optionally substituted bivalent C₁₋₁₀ saturated or partially unsaturated aliphatic chain, wherein one or more methylene unit is optionally and independently replaced with -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{B} is an optionally substituted bivalent C₁₋₁₀ saturated or partially unsaturated heteroaliphatic chain having 1-6 heteroatoms, wherein one or more methylene unit is optionally and independently replaced with -Cy-, —O—, —S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, at least methylene unit is replaced. In some embodiments, L^{B} is optionally substituted C₁₋₁₀ alkylene. In some embodiments, L^{B} is —CH₂CH₂—. In some embodiments, at least one methylene unit is replaced with —C(O)—. In some embodiments, at least one methylene unit is replaced with —C(O)N(R′)—. In some embodiments, at least one methylene unit is replaced with —N(R′)—. In some embodiments, at least one methylene unit is replaced with —NH—. In some embodiments, at least one methylene unit is replaced with -Cy-. In some embodiments, L^{B} is or comprises optionally substituted —N═CH—. In some embodiments, L^{B} is or comprises —C(O)—. In some embodiments, L^{B} is or comprises —O—. In some embodiments, L^{B} is or comprises —OC(O)—. In some embodiments, L^{B} is or comprises —CH₂OC(O)—.

In some embodiments, each -Cy- is independently an optionally substituted, 3-20 membered, monocyclic, bicyclic or polycyclic, saturated, partially saturated or aromatic ring having 0-10 heteroatoms. Suitable monocyclic unit(s) of -Cy- are described herein. In some embodiments, -Cy- is monocyclic. In some embodiments, -Cy- is bicyclic. In some embodiments, -Cy- is polycyclic. In some embodiments, -Cy-is an optionally substituted bivalent 3-10 membered monocyclic, saturated or partially unsaturated ring having 0-5 heteroatoms. In some embodiments, -Cy- is an optionally substituted bivalent 5-10 membered aromatic ring having 0-5 heteroatoms. In some embodiments, -Cy- is optionally substituted phenylene. In some embodiments, -Cy- is phenylene.

In some embodiments, R′ is R. In some embodiments, R′ is —C(O)R. In some embodiments, R′ is —C(O)OR. In some embodiments, R′ is —C(O)N(R)₂. In some embodiments, R′ is —SO₂R.

In some embodiments, R′ in various structures is a protecting group (e.g., for amino, hydroxyl, etc.), e.g., one suitable for oligonucleotide synthesis. In some embodiments, R is optionally substituted phenyl. In some embodiments, R is phenyl. In some embodiments, R is 4-nitrophenyl. In some embodiments, R is —CH₂CH₂-(4-nitrophenyl). In some embodiments, R′ is —C(O)NPh₂.

In some embodiments, each R is independently —H, or an optionally substituted group selected from C₁₋₂₀ aliphatic, C₁₋₂₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-20 membered heteroaryl having 1-10 heteroatoms, and 3-30 membered heterocyclyl having 1-10 heteroatoms. In some embodiments, two R groups are optionally and independently taken together to form a covalent bond. In some embodiments, two or more R groups on the same atom are optionally and independently taken together with the atom to form an optionally substituted, 3-20 membered, monocyclic, bicyclic or polycyclic ring having, in addition to the atom, 0-10 heteroatoms. In some embodiments, two groups on the same atom are optionally and independently taken together with the atom to form an optionally substituted, 3-20 membered, monocyclic, bicyclic or polycyclic ring having, in addition to the atom, 0-10 heteroatoms. In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms. In some embodiments, two groups on two or more atoms are optionally and independently taken together with their intervening atoms to form an optionally substituted, 3-30 membered, monocyclic, bicyclic or polycyclic ring having, in addition to the intervening atoms, 0-10 heteroatoms. In some embodiments, a formed ring is monocyclic. In some embodiments, a formed ring is bicyclic. In some embodiments, a formed ring is polycyclic. In some embodiments, each monocyclic ring unit is independently 3-10 (e.g., 3-8, 3-7, 3-6, 5-10, 5-8, 5-7, 5-6, 3, 4, 5, 6, 7, 8, 9, or 10, etc.) membered, and is independently saturated, partially saturated, or aromatic, and independently has 0-5 heteroatom. In some embodiments, a ring is saturated. In some embodiments, a ring is partially saturated. In some embodiments, a ring is aromatic. In some embodiments, a formed ring has 1-5 heteroatom. In some embodiments, a formed ring has 1 heteroatom. In some embodiments, a formed ring has 2 heteroatoms. In some embodiments, a heteroatom is nitrogen. In some embodiments, a heteroatom is oxygen.

In some embodiments, R is —H.

In some embodiments, R is optionally substituted C₁₋₂₀, C₁₋₁₅, C₁₋₁₀, C₁₋₈, C₁₋₆, C₁₋₅, C₁₋₄, C₁₋₃, or C₁₋₂ aliphatic. In some embodiments, R is optionally substituted alkyl. In some embodiments, R is optionally substituted C₁₋₆ alkyl. In some embodiments, R is optionally substituted methyl. In some embodiments, R is optionally substituted cycloaliphatic. In some embodiments, R is optionally substituted cycloalkyl.

In some embodiments, R is optionally substituted C₁₋₂₀ heteroaliphatic having 1-10 heteroatoms.

In some embodiments, R is optionally substituted C₆₋₂₀ aryl. In some embodiments, R is optionally substituted phenyl. In some embodiments, R is phenyl.

In some embodiments, R is optionally substituted C₆₋₂₀ arylaliphatic. In some embodiments, R is optionally substituted C₆₋₂₀ arylalkyl. In some embodiments, R is benzyl. In some embodiments, R is optionally substituted C₆₋₂₀ arylheteroaliphatic having 1-10 heteroatoms.

In some embodiments, R is optionally substituted 5-20 membered heteroaryl having 1-10 heteroatoms. In some embodiments, R is optionally substituted 5-membered heteroaryl having 1-4 heteroatoms. In some embodiments, R is optionally substituted 6-membered heteroaryl having 1-4 heteroatoms. In some embodiments, R is optionally substituted 3-20 membered heterocyclyl having 1-10 heteroatoms. In some embodiments, R is optionally substituted 3-10 membered heterocyclyl having 1-5 heteroatoms. In some embodiments, R is optionally substituted 5-6 membered heterocyclyl having 1-5 heteroatoms. In some embodiments, a heterocyclyl is saturated. In some embodiments, a heterocyclyl is partially saturated.

In some embodiments, a heteroatom is selected from boron, nitrogen, oxygen, sulfur, silicon and phosphorus. In some embodiments, a heteroatom is selected from nitrogen, oxygen, sulfur, and silicon. In some embodiments, a heteroatom is selected from nitrogen, oxygen, and sulfur. In some embodiments, a heteroatom is nitrogen. In some embodiments, a heteroatom is oxygen. In some embodiments, a heteroatom is sulfur.

As appreciated by those skilled in the art, embodiments described for variables can be readily combined to provide various structures. Those skilled in the art also appreciates that embodiments described for a variable can be readily utilized for other variables that can be that variable, e.g., embodiments of R for R′ R^{B2}, R^{B3}, R^{B4}, R^{B5}, R^{B6}, R^{B2′}, R^{B3′}, R^{B4′}, R^{B5′}, R^{B6′}, etc.; embodiments of embodiments of L^{B} for L^{B2}, L^{B3}, L^{B4}, L^{B5}, L^{B6}, L^{B2′}, L^{B3′}, L^{B4′}, L^{B5′}, L^{B6′}, etc. Exemplary embodiments and combinations thereof include but are not limited to structures exemplified herein. Certain examples are described below.

For example, in some embodiments, Ring BA is optionally substituted or protected

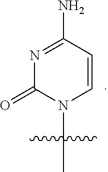 [SMILES: *n1ccc(N)nc1=O]

In some embodiments, Ring BA is

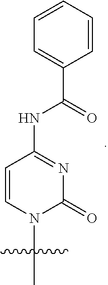 [SMILES: *n1ccc(NC(=O)c2ccccc2)nc1=O]

In some embodiments, Ring BA is

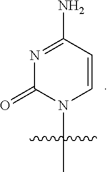 [SMILES: *n1ccc(N)nc1=O]

In some embodiments, X⁴ is —C(O)—, and O in —C(O)— of X⁴ may form a hydrogen bond with a —H of R⁵, e.g., a —H in —NHR′, —OH, or —SH of R^{5′}. In some embodiments, X⁴ is —C(O)—, and X⁵ is —C(R⁵)═. In some embodiments, R^{5′} is —NHR′. In some embodiments, R⁵ is -L^{B5}-NHR′. In some embodiments, L^{B5} is optionally substituted —CH₂—. In some embodiments, a methylene unit is replaced with —C(O)—. In some embodiments, L^{B5} is —C(O)—. In some embodiments, R′ is optionally substituted methyl. In some embodiments, R′ is —CH₂Ph. In some embodiments, R′ is optionally substituted phenyl. In some embodiments, R′ is phenyl. In some embodiments, R′ is optionally substituted C₁₋₆ aliphatic. In some embodiments, R′ is optionally substituted C₁_₆ alkyl. In some embodiments, R′ is optionally substituted methyl. In some embodiments, R′ is methyl. In some embodiments, Ring BA is optionally protected

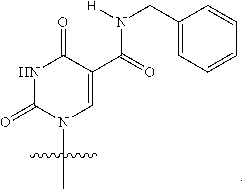 [SMILES: *n1cc(C(=O)NCc2ccccc2)c(=O)[nH]c1=O]

In some embodiments, Ring BA is

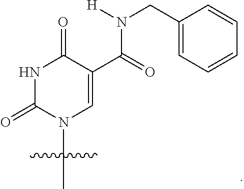 [SMILES: *n1cc(C(=O)NCc2ccccc2)c(=O)[nH]c1=O]

In some embodiments, Ring BA is optionally protected

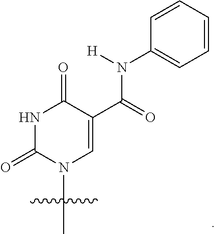 [SMILES: *n1cc(C(=O)Nc2ccccc2)c(=O)[nH]c1=O]

In some embodiments, Ring BA is

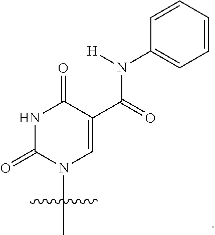 [SMILES: *n1cc(C(=O)Nc2ccccc2)c(=O)[nH]c1=O]

In some embodiments, Ring BA is optionally protected

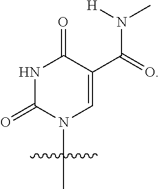 [SMILES: *n1cc(C(=O)NC)c(=O)[nH]c1=O]

In some embodiments, Ring BA is

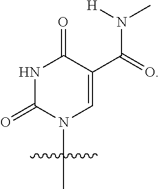 [SMILES: *n1cc(C(=O)NC)c(=O)[nH]c1=O]

In some embodiments, Ring BA is optionally protected

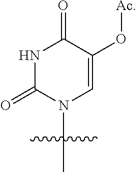 [SMILES: *n1cc(OC(C)=O)c(=O)[nH]c1=O]

In some embodiments, Ring BA is

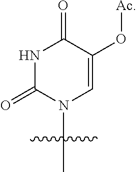 [SMILES: *n1cc(OC(C)=O)c(=O)[nH]c1=O]

In some embodiments, Ring BA is optionally protected

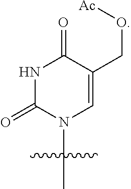 [SMILES: *n1cc(COC(C)=O)c(=O)[nH]c1=O]

In some embodiments, Ring BA is

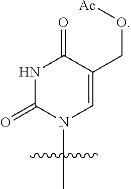 [SMILES: *n1cc(COC(C)=O)c(=O)[nH]c1=O]

In some embodiments, Ring BA is optionally protected

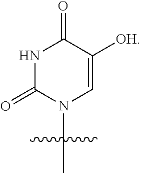 [SMILES: *n1cc(O)c(=O)[nH]c1=O]

In some embodiments, Ring BA is

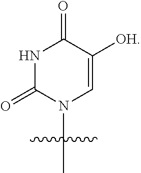 [SMILES: *n1cc(O)c(=O)[nH]c1=O]

In some embodiments, Ring BA is optionally protected

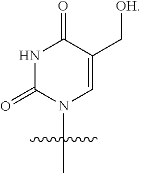 [SMILES: *n1cc(CO)c(=O)[nH]c1=O]

In some embodiments, Ring BA is

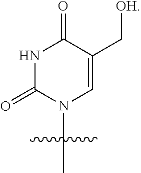 [SMILES: *n1cc(CO)c(=O)[nH]c1=O]

In some embodiments, X¹ is —C(−)=, and X⁴ is ═C(—N(R^{B4})₂)—. In some embodiments, two R groups on the same atom, e.g., a nitrogen atom, are taken together to form optionally substituted ═CH₂ or ═NH. In some embodiments, two R groups on the same atom, e.g., a nitrogen atom, are taken together to form optionally substituted ═C(-L^{B4}-R)₂, ═N-L^{B4}-R. In some embodiments, a formed group is ═CHN(R)₂. In some embodiments, a formed group is ═CHN(CH₃)₂. In some embodiments, X⁴ is ═C(—N═CHN(CH₃)₂)—. In some embodiments, —N(R^{B4})₂ is —NR^{B4}. In some embodiments, R^{B4} is —NHC(O)R. In some embodiments Ring BA is optionally substituted or protected

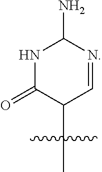 [SMILES: *C1C=NC(N)NC1=O]

In some embodiments, Ring BA is

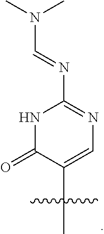 [SMILES: *c1cnc(/N=C/N(C)C)[nH]c1=O]

In some embodiments, Ring BA is

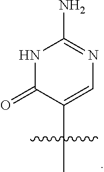 [SMILES: *c1cnc(N)[nH]c1=O]

In some embodiments, X¹ is —N(−)-, X² is —C(O)—, and X³ is —N(R^{B3})—. In some embodiments, X¹ is —N(−)-, X² is —C(O)—, X³ is —N(R^{B3})—, and X⁴ is —C(R^{B4})═. In some embodiments, X¹ is —N(−)-, X² is —C(O)—, X³ is —N(R^{B3})—, X⁴ is —C(R^{B4})═, and X⁵ is —C(R^{B5})═. In some embodiments, Ring BA is optionally substituted or protected

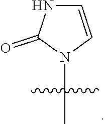 [SMILES: *n1cc[nH]c1=O]

In some embodiments, Ring BA is

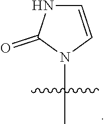 [SMILES: *n1cc[nH]c1=O]

In some embodiments, X³ is —N(R′)—. In some embodiments, R′ is —C(O)R. In some embodiments, X⁴ is —C(R^{B4})₂—. In some embodiments, R^{B4} is —R. In some embodiments, R^{B4} is —H. In some embodiments, X⁴ is —CH₂—. In some embodiments, X⁵ is —C(R^{B5})₂—. In some embodiments, R^{B5} is —R. In some embodiments, R^{B5} is —H. In some embodiments, X⁵ is —CH₂—. In some embodiments, Ring BA is optionally substituted or protected

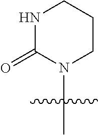 [SMILES: *N1CCCNC1=O]

In some embodiments, Ring BA is

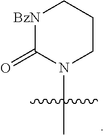

In some embodiments, Ring BA is

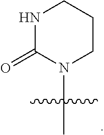 [SMILES: *N1CCCNC1=O]

In some embodiments, X⁴ is —C(R^{B4})═. In some embodiments, X⁴ is —CH═. In some embodiments, X⁵ is —C(R^{B5})═. In some embodiments, X⁵ is —CH═. In some embodiments, Ring BA is optionally substituted or protected

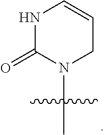 [SMILES: *N1CC=CNC1=O]

In some embodiments, Ring BA is

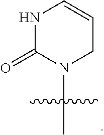 [SMILES: *N1CC=CNC1=O]

In some embodiments, Ring BA is optionally substituted or protected

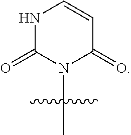 [SMILES: *n1c(=O)cc[nH]c1=O]

In some embodiments, Ring BA is

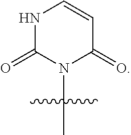 [SMILES: *n1c(=O)cc[nH]c1=O]

In some embodiments, X⁴ is —C(R^{B4})₂—. In some embodiments, X⁴ is —CH₂—. In some embodiments, X⁵ is —C(R^{B5})═. In some embodiments, X⁵ is —CH═. In some embodiments, Ring BA is optionally substituted or protected

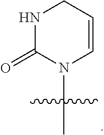 [SMILES: *N1C=CCNC1=O]

In some embodiments, Ring BA is

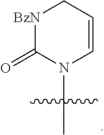

In some embodiments, Ring BA is

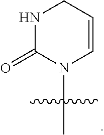 [SMILES: *N1C=CCNC1=O]

In some embodiments, X¹ is —N(−)-, X² is —C(O)—, X³ is —N(R^{B3})—, X⁴ is —C(R^{B4})═, X⁵ is —C(R^{B5})═, X⁶ is —C(O)—. In some embodiments, each of R^{B3}, R^{B4} and R^{B5} is independently R. In some embodiments, R^{B3} is —H. In some embodiments, R^{B4} is —H. In some embodiments, R^{B5} is —H. In some embodiments, BA is or comprises optionally substituted or protected

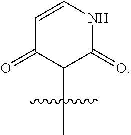 [SMILES: *C1C(=O)C=CNC1=O]

In some embodiments, BA is

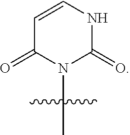 [SMILES: *n1c(=O)cc[nH]c1=O]

In some embodiments, X¹ is —N(−)-, X² is —C(O)—, X³ is —N(R^{B3})—. In some embodiments, X⁴ is —C(R^{B4})₂—, wherein the two R^{B4} are taken together to form ═O, or ═C(-L^{B4}-R^{B41})₂, ═N-L^{B4}-R^{B41}. In some embodiments, X⁴ is —C(═NR^{B4})—. In some embodiments, X⁵ is —C(R^{B5})═. In some embodiments, R^{B41} or R^{B4} and R^{B5} are R, and are taken together with their intervening atoms to form an optionally substituted ring as described herein. In some embodiment, Ring BA is optionally substituted or protected

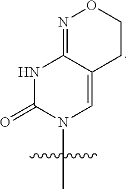 [SMILES: *N1C=C2CCON=C2NC1=O]

In some embodiment, Ring BA is

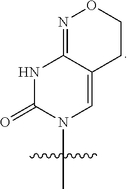 [SMILES: *N1C=C2CCON=C2NC1=O]

In some embodiment, Ring BA is optionally substituted or protected

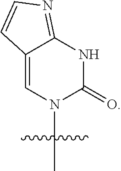 [SMILES: *n1cc2ccnc-2[nH]c1=O]

In some embodiment, Ring BA is

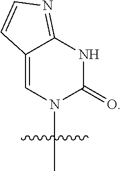 [SMILES: *n1cc2ccnc-2[nH]c1=O]

In some embodiments, X¹ is —N(−)-, X² is —C(O)—, X³ is —N═. In some embodiments, X⁴ is —C(—N(R^{B4})₂)═. In some embodiments, X⁴ is —C(—NHR^{B4})═. In some embodiments, X⁵ is —C(R^{B5})═. In some embodiments, one R^{B4} and R^{B5} are taken together to form an optionally substituted ring as described herein. In some embodiments, a formed ring is an optionally substituted 5-membered ring having a nitrogen atom. In some embodiment, Ring BA is optionally substituted or protected

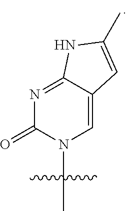 [SMILES: *n1cc2cc(C)[nH]c2nc1=O]

In some embodiment, Ring BA is

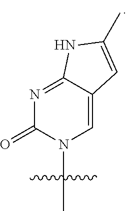 [SMILES: *n1cc2cc(C)[nH]c2nc1=O]

In some embodiment, Ring BA is optionally substituted or protected

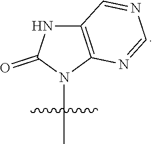 [SMILES: *n1c(=O)[nH]c2cncnc21]

In some embodiment, Ring BA is

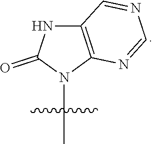 [SMILES: *n1c(=O)[nH]c2cncnc21]

In some embodiment, Ring BA is optionally substituted or protected

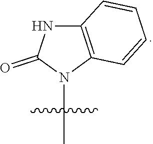 [SMILES: *n1c(=O)[nH]c2ccccc21]

In some embodiment, Ring BA is

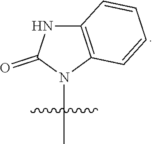 [SMILES: *n1c(=O)[nH]c2ccccc21]

In some embodiment, Ring BA is optionally substituted or protected

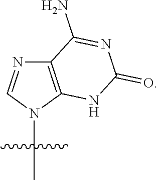 [SMILES: *n1cnc2c(N)nc(=O)[nH]c21]

In some embodiment, Ring BA is

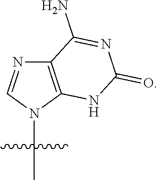 [SMILES: *n1cnc2c(N)nc(=O)[nH]c21]

In some embodiments, Ring BA has the structure of formula BA-IV or BA-V. In some embodiments, X¹ is —N(−)-, X² is —C(O)—, and X³ is —N═. In some embodiments, X¹ is —N(−)-, X² is —C(O)—, X³ is —N═, and X⁶ is —C(R^{B6})═. In some embodiments, Ring BA^{A} is 5-6 membered. In some embodiments, Ring BA^{A} is monocyclic. In some embodiments, Ring BA^{A} is partially unsaturated. In some embodiments, Ring BA^{A} is aromatic. In some embodiments, Ring BA^{A} has 0-2 heteroatoms. In some embodiments, Ring BA^{A} has 1-2 heteroatoms. In some embodiments, Ring BA^{A} has one heteroatom. In some embodiments, Ring BA^{A} has 2 heteroatoms. In some embodiments, a heteroatom is nitrogen. In some embodiments, heteroatom is oxygen. In some embodiments, Ring BA is optionally substituted or protected

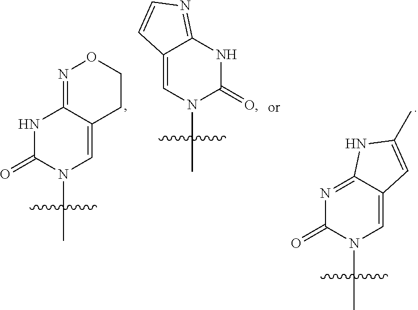

In some embodiments, Ring BA is

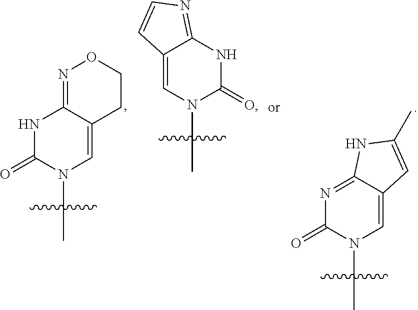

In some embodiments, Ring BA is an optionally substituted 5-membered ring. In some embodiments, X¹ is bonded to X⁵. In some embodiments, each of X⁴ and X⁵ is independently —CH═. In some embodiments, X¹ is —N(−)-, X² is —C(O)—, X³ is —NH—, X⁴ is —CH═, and X⁵ is —CH═. In some embodiments, Ring BA is optionally substituted or protected

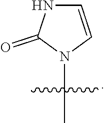 [SMILES: *n1cc[nH]c1=O]

In some embodiments, Ring BA is

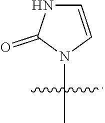 [SMILES: *n1cc[nH]c1=O]

In some embodiments, Ring BA has the structure of formula BA-VI. In some embodiments, X^{1′} is —N(−)-, X^{2′} is —C(O)— and X^{3′} is —N(R^{B3})—. In some embodiments, X^{1′} is —N(−)-, X^{2′} is —C(O)—, X^{3′} is —N(R^{B3})—, X^{4′} is —C(R^{B4′})═, X^{5′} is —N═, X^{6′} is —C(R^{B6′})═, and X⁷ is —N═. In some embodiments, X^{1′} is —N(−)-, X^{2′} is —C(O)—, X^{3′} is —N(R^{B3})—, X^{4′} is —C(R^{B4′})═, X^{5′} is —C(R^{B5′})═, X^{6′} is —C(R^{B6′})═, and X^{7′} is —C(R^{B7′})═. In some embodiments, Ring BA is optionally substituted or protected

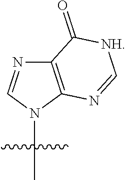 [SMILES: *n1cnc2c(=O)[nH]cnc21]

In some embodiments, Ring BA is

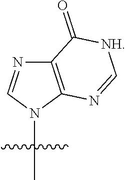 [SMILES: *n1cnc2c(=O)[nH]cnc21]

In some embodiments, Ring BA is optionally substituted or protected

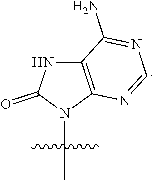 [SMILES: *n1c(=O)[nH]c2c(N)ncnc21]

In some embodiments, Ring BA is

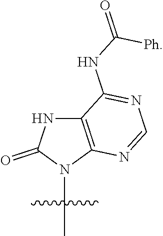 [SMILES: *n1c(=O)[nH]c2c(NC(=O)c3ccccc3)ncnc21]

In some embodiments, Ring BA is

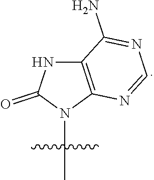 [SMILES: *n1c(=O)[nH]c2c(N)ncnc21]

In some embodiments, Ring BA is optionally substituted or protected

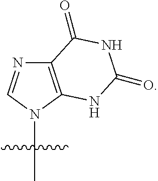 [SMILES: *n1cnc2c(=O)[nH]c(=O)[nH]c21]

In some embodiments, Ring BA is

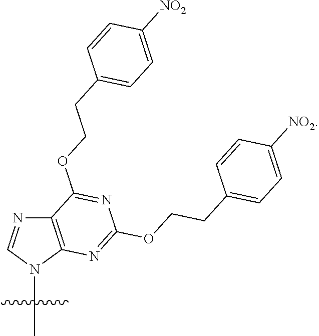 [SMILES: *n1cnc2c(OCCc3ccc([N+](=O)[O-])cc3)nc(OCCc3ccc([N+](=O)[O-])cc3)nc21]

In some embodiments, Ring BA is

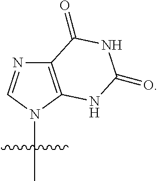 [SMILES: *n1cnc2c(=O)[nH]c(=O)[nH]c21]

In some embodiments, Ring BA is optionally substituted or protected

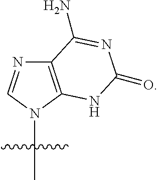 [SMILES: *n1cnc2c(N)nc(=O)[nH]c21]

In some embodiments, Ring BA is

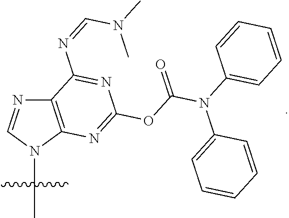 [SMILES: *n1cnc2c(/N=C\N(C)C)nc(OC(=O)N(c3ccccc3)c3ccccc3)nc21]

In some embodiments, Ring BA is

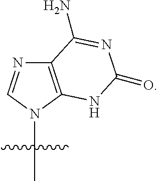 [SMILES: *n1cnc2c(N)nc(=O)[nH]c21]

In some embodiments Ring BA is optionally substituted or protected

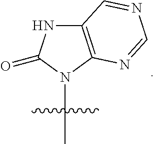 [SMILES: *n1c(=O)[nH]c2cncnc21]

In some embodiments, Ring BA is

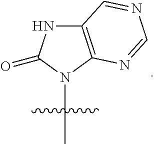 [SMILES: *n1c(=O)[nH]c2cncnc21]

In some embodiments, Ring BA is optionally substituted or protected

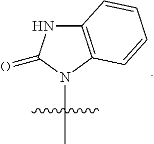 [SMILES: *n1c(=O)[nH]c2ccccc21]

In some embodiments, Ring BA is

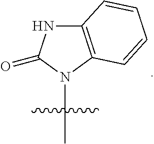 [SMILES: *n1c(=O)[nH]c2ccccc21]

In some embodiments, X^{1′} is —N(−)-, X^{2′} is —C(R^{B2′})═, and X^{3′} is —N═. In some embodiments, X^{1′} is —N(−)-, X^{2′} is —C(R^{B2′})═, X^{3′} is —N═, X^{4′} is —C(—N(R^{B4′})₂)═, X^{5′} is —N═, X^{6′} is —C(O)—, and X^{7′} is —N(R^{B7′})—. In some embodiments, Ring BA is optionally substituted or protected

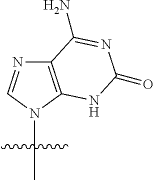 [SMILES: *n1cnc2c(N)nc(=O)[nH]c21]

In some embodiments, Ring BA is

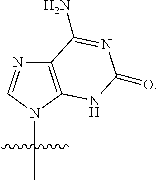 [SMILES: *n1cnc2c(N)nc(=O)[nH]c21]

In some embodiments, X¹ is —C(−)=, X² is —C(O)—, and X³ is —N(R^{B3})—. In some embodiments, X¹ is —C(−)=, X² is —C(O)—, X³ is —N(R^{B3})—, —C(—N(R^{B4})₂)═, and X⁴ is —C(R^{B4})═. In some embodiments, X¹ is —C(−)=, X² is —C(O)—, X³ is —N(R^{B3})—, —C(—N(R^{B4})₂)═, X⁴ is —C(R^{B4})═, and X⁶ is —C(R^{B6})═. In some embodiments, each of R^{B3}, R^{B4}, and R^{B6} is independently —H. In some embodiments, Ring BA is optionally substituted or protected

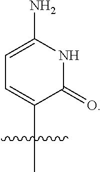 [SMILES: *c1ccc(N)[nH]c1=O]

In some embodiments, Ring BA is

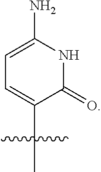 [SMILES: *c1ccc(N)[nH]c1=O]

In some embodiments, Ring BA is optionally substituted or protected

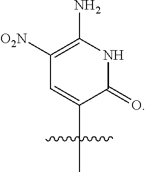 [SMILES: *c1cc([N+](=O)[O-])c(N)[nH]c1=O]

In some embodiments, Ring BA is

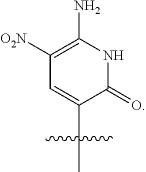 [SMILES: *c1cc([N+](=O)[O-])c(N)[nH]c1=O]

As described herein, Ring BA may be optionally substituted. In some embodiments, each of X², X³, X⁴, X⁵, X⁶, X², X^{3′}, X^{4′}, X^{5′}, X^{6′}, and X^{7′} is independently and optionally substituted when it is —CH═, —C(OH)═, —C(—NH₂)═, —CH₂—, —C(═NH)—, or —NH—. In some embodiments, each of X², X³, X⁴, X⁵, X⁶, X^{2′}, X^{3′}, X^{4′}, X^{5′}, X^{6′}, and X^{7′} is independently and optionally substituted when it is —CH═, —CH₂—, or —NH—. In some embodiments, each of X², X³, X⁴, X⁵, X⁶, X^{2′}, X^{3′}, X^{4′}, X^{5′}, X^{6′}, and X^{7′} is independently and optionally substituted when it is —CH═. In some embodiments, each of X², X³, X⁴, X⁵, X⁶, X^{2′}, X^{3′}, X^{4′}, X^{5′}, X^{6′}, and X^{7′} is independently and optionally substituted when it is —CH₂—. In some embodiments, each of X², X³, X⁴, X⁵, X⁶, X^{2′}, X^{3′}, X^{4′}, X^{5′}, X^{6′}, and X^{7′} is independently and optionally substituted when it is —NH—. In some embodiments, X² is optionally substituted —CH═, —C(OH)═, —C(—NH₂)═, —CH₂—, —C(═NH)—, or —NH—. In some embodiments, X³ is optionally substituted —CH═, —C(OH)═, —C(—NH₂)═, —CH₂—, —C(═NH)—, or —NH—. In some embodiments, X⁴ is optionally substituted —CH═, —C(OH)═, —C(—NH₂)═, —CH₂—, —C(═NH)—, or —NH—. In some embodiments, X⁵ is optionally substituted —CH═, —C(OH)═, —C(—NH₂)═, —CH₂—, —C(═NH)—, or —NH—. In some embodiments, X⁶ is optionally substituted —CH═, —C(OH)═, —C(—NH₂)═, —CH₂—, —C(═NH)—, or —NH—. In some embodiments, XT is optionally substituted —CH═, —C(OH)═, —C(—NH₂)═, —CH₂—, —C(═NH)—, or —NH—. In some embodiments, X^{3′} is optionally substituted —CH═, —C(OH)═, —C(—NH₂)═, —CH₂—, —C(═NH)—, or —NH—. In some embodiments, X^{4′} is optionally substituted —CH═, —C(OH)═, —C(—NH₂)═, —CH₂—, —C(═NH)—, or —NH—. In some embodiments, X^{5′} is optionally substituted —CH═, —C(OH)═, —C(—NH₂)═, —CH₂—, —C(═NH)—, or —NH—. In some embodiments, X^{6′} is optionally substituted —CH═, —C(OH)═, —C(—NH₂)═, —CH₂—, —C(═NH)—, or —NH—. In some embodiments, XT is optionally substituted —CH═, —C(OH)═, —C(—NH₂)═, —CH₂—, —C(═NH)—, or —NH—.

As demonstrated herein, in some embodiments provided oligonucleotides comprising certain nucleobases (e.g., b001A, b002A, b008U, C, A, etc.) opposite to target adenosines can among other things provide improved editing efficiency (e.g., compared to a reference nucleobase such as U). In some embodiments, an opposite nucleoside is linked to an I to its 3′ side.

In some embodiments, an opposite nucleoside is abasic, e.g., having the structure of L010

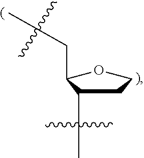

### L012

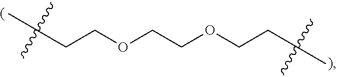 [SMILES: *CCOCCOCC*]

### or L028

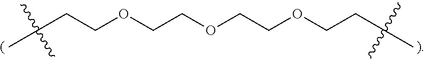 [SMILES: *CCOCCOCCOCC*]

As appreciated by those skilled in the art and demonstrated in various oligonucleotides, abasic nucleosides may also be utilized in other portions of oligonucleotides, and oligonucleotides may comprise one or more (e.g., 1, 2, 3, 4, 5, or more), optionally consecutive, abasic nucleosides. In some embodiments, a first domain comprises one or more optionally consecutive, abasic nucleosides. In some embodiments, an oligonucleotide comprises one and no more than one abasic nucleoside. In some embodiments, each abasic nucleoside is independently in a first domain or a first subdomain of a second domain. In some embodiments, each abasic nucleoside is independently in a first domain. In some embodiments, each abasic nucleoside is independently in a first subdomain of a second domain. In some embodiments, an abasic nucleoside is opposite to a target adenosine. As demonstrated herein, a single abasic nucleoside may replace one or more nucleosides each of which independently comprises a nucleobase in a reference oligonucleotide, for example, L010 may be utilized to replace 1 nucleoside which comprises a nucleobase, L012 may be utilized to replace 1, 2 or 3 nucleosides each of which independently comprises a nucleobase, and L028 may be utilized to replace 1, 2 or 3 nucleosides each of which independently comprises a nucleobase. In some embodiments, a basic nucleoside is linked to its 3′ immediate nucleoside (which is optionally abasic) through a stereorandom linkage (e.g., a stereorandom phosphorothioate internucleotidic linkage). In some embodiments, each basic nucleoside is independently linked to its 3′ immediate nucleoside (which is optionally abasic) through a stereorandom linkage (e.g., a stereorandom phosphorothioate internucleotidic linkage).

In some embodiments, a modified nucleobase opposite to a target adenine can greatly improve properties and/or activities of an oligonucleotide. In some embodiments, a modified nucleoase at the opposite position can provide high activities even when there is a G next to it (e.g., at the 3′ side), and/or other nucleobases, e.g. C, provide much lower activities or virtually no detect activities.

In some embodiments, a second domain comprises one or more sugars comprising two 2′-H (e.g., natural DNA sugars). In some embodiments, a second domain comprises one or more sugars comprising 2′-OH (e.g., natural RNA sugars). In some embodiments, a second domain comprises one or more modified sugars. In some embodiments, a modified sugar comprises a 2′-modification. In some embodiments, a modified sugar is a bicyclic sugar, e.g., a LNA sugar. In some embodiments, a modified sugar is an acyclic sugar (e.g., by breaking a C2-C3 bond of a corresponding cyclic sugar).

In some embodiments, a second domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars. In some embodiments, a second domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently bicyclic sugars (e.g., a LNA sugar) or a 2′-OR modified sugars, wherein R is independently optionally substituted C₁₋₆ aliphatic. In some embodiments, a second domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently 2′-OR modified sugars, wherein R is independently optionally substituted C₁₋₆ aliphatic. In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15. In some embodiments, the number is 16. In some embodiments, the number is 17. In some embodiments, the number is 18. In some embodiments, the number is 19. In some embodiments, the number is 20. In some embodiments, R is methyl.

In some embodiments, about 5%-100%, (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a second domain are independently a modified sugar. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a second domain are independently a bicyclic sugar (e.g., a LNA sugar) or a 2′-OR modified sugar, wherein R is independently optionally substituted C₁₋₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a second domain are independently a 2′-OR modified sugar, wherein R is independently optionally substituted C₁₋₆ aliphatic. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, R is methyl.

In some embodiments, a second domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a second domain are independently modified sugars with a modification that is not 2′-F. In some embodiments, about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in a second domain are independently modified sugars with a modification that is not 2′-F. In some embodiments, modified sugars of a second domain are each independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, a second domain comprises one or more 2′-F modified sugars. In some embodiments, a second domain comprises no 2′-F modified sugars. In some embodiments, a second domain comprises one or more bicyclic sugars and/or 2′-OR modified sugars wherein R is not —H. In some embodiments, levels of bicyclic sugars and/or 2′-OR modified sugars wherein R is not —H, individually or combined, are relatively high compared to level of 2′-F modified sugars. In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a second domain comprises 2′-F. In some embodiments, no more than about 50% of sugars in a second domain comprises 2′-F. In some embodiments, a second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification. In some embodiments, a second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification. In some embodiments, a second domain comprises one or more (e.g., about 1-20, 1, 2, 3, 4, 5, 6, 7, 8, 9, 10, 11, 12, 13, 14, 15, 16, 17, 18, 19, 20, etc.) bicyclic sugars, e.g., LNA sugars. In some embodiments, a second domain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).

In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a second domain comprises 2′-MOE. In some embodiments, no more than about 50% of sugars in a second domain comprises 2′-MOE. In some embodiments, no sugars in a second domain comprises 2′-MOE.

In some embodiments, a second domain comprise about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in a second domain are modified internucleotidic linkages. In some embodiments, each internucleotidic linkage in a second domain is independently a modified internucleotidic linkage. In some embodiments, each modified internucleotidic linkages is independently a chiral internucleotidic linkage. In some embodiments, a modified or chiral internucleotidic linkage is a phosphorothioate internucleotidic linkage. In some embodiments, a modified or chiral internucleotidic linkage is a non-negatively charged internucleotidic linkage. In some embodiments, a modified or chiral internucleotidic linkage is a neutral internucleotidic linkage, e.g., n001. In some embodiments, each modified internucleotidic linkages is independently a phosphorothioate internucleotidic linkage or a non-negatively charged internucleotidic linkage. In some embodiments, each modified internucleotidic linkages is independently a phosphorothioate internucleotidic linkage or a neutral internucleotidic linkage. In some embodiments, each modified internucleotidic linkages is independently a phosphorothioate internucleotidic linkage. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a second domain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a second domain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a second domain is chirally controlled. In some embodiments, each is independently chirally controlled. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a second domain is Sp. In some embodiments, each is independently chirally controlled. In some embodiments, at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) phosphorothioate internucleotidic linkages in a second domain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a second domain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a second domain is Sp. In some embodiments, the number is one or more. In some embodiments, the number is 2 or more. In some embodiments, the number is 3 or more. In some embodiments, the number is 4 or more. In some embodiments, the number is 5 or more. In some embodiments, the number is 6 or more. In some embodiments, the number is 7 or more. In some embodiments, the number is 8 or more. In some embodiments, the number is 9 or more. In some embodiments, the number is 10 or more. In some embodiments, the number is 11 or more. In some embodiments, the number is 12 or more. In some embodiments, the number is 13 or more. In some embodiments, the number is 14 or more. In some embodiments, the number is 15 or more. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, each internucleotidic linkage linking two second domain nucleosides is independently a modified internucleotidic linkage. In some embodiments, each modified internucleotidic linkages is independently a chiral internucleotidic linkage. In some embodiments, each modified internucleotidic linkages is independently a phosphorothioate internucleotidic linkage. In some embodiments, each chiral internucleotidic linkage is independently a phosphorothioate internucleotidic linkage. In some embodiments, each modified internucleotidic linkages is independently a Sp chiral internucleotidic linkage. In some embodiments, each modified internucleotidic linkages is independently a Sp phosphorothioate internucleotidic linkage. In some embodiments, each chiral internucleotidic linkages is independently a Sp phosphorothioate internucleotidic linkage. In some embodiments, an internucleotidic linkage of a second domain is bonded to two nucleosides of the second domain. In some embodiments, an internucleotidic linkage bonded to a nucleoside in a first domain and a nucleoside in a second domain may be properly considered an internucleotidic linkage of a second domain. In some embodiments, it was observed that a high percentage (e.g., relative to Rp internucleotidic linkages and/or natural phosphate linkages) of Sp internucleotidic linkages provide improved properties and/or activities, e.g., high stability and/or high adenosine editing activity.

In some embodiments, a second domain comprises a certain level of Rp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in a second domain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in a second domain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in a second domain. In some embodiments, a percentage is about or no more than about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, about 1-50, 1-40, 1-30, 1-25, 1-20, 1-15, 1-10, 1-5, e.g., about 1, 2, 3, 4, 5, 6, 7, 8, 9, 10, 11, 12, 13, 14, 15, 16, 17, 18, 19, or 20 internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10.

In some embodiments, each phosphorothioate internucleotidic linkage in a second domain is independently chirally controlled. In some embodiments, each is independently Sp or Rp. In some embodiments, a high level is Sp as described herein. In some embodiments, each phosphorothioate internucleotidic linkage in a second domain is chirally controlled and is Sp. In some embodiments, one or more, e.g., about 1-5 (e.g., about 1, 2, 3, 4, or 5) is Rp.

In some embodiments, each phosphorothioate internucleotidic linkage in a second domain is independently chirally controlled. In some embodiments, each is independently Sp or Rp. In some embodiments, a high level is Sp as described herein. In some embodiments, each phosphorothioate internucleotidic linkage in a second domain is chirally controlled and is Sp. In some embodiments, one or more, e.g., about 1-5 (e.g., about 1, 2, 3, 4, or 5) is Rp.

In some embodiments, as illustrated in certain examples, a second domain comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in a second domain is about 1-10, or about 1, 2, 3, 4, 5, 6, 7, 8, 9, or 10. In some embodiments, it is about 1. In some embodiments, it is about 2. In some embodiments, it is about 3. In some embodiments, it is about 4. In some embodiments, it is about 5. In some embodiments, two or more non-negatively charged internucleotidic linkages are consecutive. In some embodiments, no two non-negatively charged internucleotidic linkages are consecutive. In some embodiments, all non-negatively charged internucleotidic linkages in a second domain are consecutive (e.g., 3 consecutive non-negatively charged internucleotidic linkages). In some embodiments, a non-negatively charged internucleotidic linkage, or two or more (e.g., about 2, about 3, about 4 etc.) consecutive non-negatively charged internucleotidic linkages, are at the 3′-end of a second domain. In some embodiments, the last two or three or four internucleotidic linkages of a second domain comprise at least one internucleotidic linkage that is not a non-negatively charged internucleotidic linkage. In some embodiments, the last two or three or four internucleotidic linkages of a second domain comprise at least one internucleotidic linkage that is not n001.

In some embodiments, the internucleotidic linkage linking the last two nucleosides of a second domain is a non-negatively charged internucleotidic linkage. In some embodiments, the internucleotidic linkage linking the last two nucleosides of a second domain is a Sp non-negatively charged internucleotidic linkage. In some embodiments, the internucleotidic linkage linking the last two nucleosides of a second domain is a Rp non-negatively charged internucleotidic linkage. In some embodiments, the internucleotidic linkage linking the last two nucleosides of a second domain is a phosphorothioate internucleotidic linkage. In some embodiments, the internucleotidic linkage linking the last two nucleosides of a second domain is a Sp phosphorothioate internucleotidic linkage. In some embodiments, the last two nucleosides of a second domain are the last two nucleosides of an oligonucleotide. In some embodiments, the internucleotidic linkage linking the first two nucleosides of a second domain is a non-negatively charged internucleotidic linkage. In some embodiments, the internucleotidic linkage linking the first two nucleosides of a second domain is a Sp non-negatively charged internucleotidic linkage. In some embodiments, the internucleotidic linkage linking the first two nucleosides of a second domain is a Rp non-negatively charged internucleotidic linkage. In some embodiments, the internucleotidic linkage linking the first two nucleosides of a second domain is a phosphorothioate internucleotidic linkage. In some embodiments, the internucleotidic linkage linking the first two nucleosides of a second domain is a Sp phosphorothioate internucleotidic linkage. In some embodiments, a non-negatively charged internucleotidic linkage is a neutral internucleotidic linkage such as n001.

In some embodiments, a second domain comprises one or more natural phosphate linkages. In some embodiments, a second domain contains no natural phosphate linkages.

In some embodiments, a second domain recruits, promotes or contribute to recruitment of, a protein such as an ADAR protein. In some embodiments, a second domain recruits, or promotes or contribute to interactions with, a protein such as an ADAR protein. In some embodiments, a second domain contacts with a RNA binding domain (RBD) of ADAR. In some embodiments, a second domain contacts with a catalytic domain of ADAR which has a deaminase activity. In some embodiments, various nucleobases, sugars and/or internucleotidic linkages may interact with one or more residues of proteins, e.g., ADAR proteins.

In some embodiments, a second domain comprises or consists of a first subdomain as described herein. In some embodiments, a second domain comprises or consists of a second subdomain as described herein. In some embodiments, a second domain comprises or consists of a third subdomain as described herein. In some embodiments, a second domain comprises or consists of a first subdomain, a second subdomain and a third subdomain from 5′ to 3′. Certain embodiments of such subdomains are described below.

First Subdomains

As described herein, in some embodiment, an oligonucleotide comprises a first domain and a second domain from 5′ to 3′. In some embodiments, a second domain comprises or consists of a first subdomain, a second subdomain, and a third subdomain from 5′ to 3′. Certain embodiments of a first subdomain are described below as examples. In some embodiments, a first subdomain comprise a nucleoside opposite to target adenosine to be modified (e.g., conversion to I).

In some embodiments, a first subdomain has a length of about 1-50, 1-40, 1-30, 1-20 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 1, 2, 3, 4, 5, 6, 7, 8, 9, 10, 11, 12, 13, 14, 15, 16, 17, 18, 19, 20, 21, 22, 23, 24, 25, 30, 40 or 50, etc.) nucleobases. In some embodiments, a first subdomain has a length of about 5-30 nucleobases. In some embodiments, a first subdomain has a length of about 10-30 nucleobases. In some embodiments, a first subdomain has a length of about 10-20 nucleobases. In some embodiments, a first subdomain has a length of about 5-15 nucleobases. In some embodiments, a first subdomain has a length of about 13-16 nucleobases. In some embodiments, a first subdomain has a length of about 6-12 nucleobases. In some embodiments, a first subdomain has a length of about 6-9 nucleobases. In some embodiments, a first subdomain has a length of about 1-10 nucleobases. In some embodiments, a first subdomain has a length of about 1-7 nucleobases. In some embodiments, a first subdomain has a length of 1 nucleobase. In some embodiments, a first subdomain has a length of 2 nucleobases. In some embodiments, a first subdomain has a length of 3 nucleobases. In some embodiments, a first subdomain has a length of 4 nucleobases. In some embodiments, a first subdomain has a length of 5 nucleobases. In some embodiments, a first subdomain has a length of 6 nucleobases. In some embodiments, a first subdomain has a length of 7 nucleobases. In some embodiments, a first subdomain has a length of 8 nucleobases. In some embodiments, a first subdomain has a length of 9 nucleobases. In some embodiments, a first subdomain has a length of 10 nucleobases. In some embodiments, a first subdomain has a length of 11 nucleobases. In some embodiments, a first subdomain has a length of 12 nucleobases. In some embodiments, a first subdomain has a length of 13 nucleobases. In some embodiments, a first subdomain has a length of 14 nucleobases. In some embodiments, a first subdomain has a length of 15 nucleobases.

In some embodiments, a first subdomain is about, or at least about, 5-95%, 10%-90%, 20%-80%, 30%-70%, 40%-70%, 40%-60%, 5%, 10%, 15%, 20%, 25%, 30%, 35%, 40%, 45%, 50%, 55%, 60%, 65%, 70%, 75%, 80%, 85%, 90%, 95% of a second domain. In some embodiments, a percentage is about 30%-80%. In some embodiments, a percentage is about 30%-70%. In some embodiments, a percentage is about 40%-60%. In some embodiments, a percentage is about 20%. In some embodiments, a percentage is about 25%. In some embodiments, a percentage is about 30%. In some embodiments, a percentage is about 35%. In some embodiments, a percentage is about 40%. In some embodiments, a percentage is about 45%. In some embodiments, a percentage is about 50%. In some embodiments, a percentage is about 55%. In some embodiments, a percentage is about 60%. In some embodiments, a percentage is about 65%. In some embodiments, a percentage is about 70%. In some embodiments, a percentage is about 75%. In some embodiments, a percentage is about 80%. In some embodiments, a percentage is about 85%. In some embodiments, a percentage is about 90%.

In some embodiments, one or more (e.g., 1-20, 1, 2, 3, 4, 5, 6, 7, 8, 9, or 10, etc.) mismatches exist in a first subdomain when an oligonucleotide is aligned with a target nucleic acid for complementarity. In some embodiments, there is 1 mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches.

In some embodiments, one or more (e.g., 1-20, 1, 2, 3, 4, 5, 6, 7, 8, 9, or 10, etc.) wobbles exist in a first subdomain when an oligonucleotide is aligned with a target nucleic acid for complementarity. In some embodiments, there is 1 wobble. In some embodiments, there are 2 wobbles. In some embodiments, there are 3 wobbles. In some embodiments, there are 4 wobbles. In some embodiments, there are 5 wobbles. In some embodiments, there are 6 wobbles. In some embodiments, there are 7 wobbles. In some embodiments, there are 8 wobbles. In some embodiments, there are 9 wobbles. In some embodiments, there are 10 wobbles.

In some embodiments, duplexes of oligonucleotides and target nucleic acids in a first subdomain region comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, a first subdomain is fully complementary to a target nucleic acid.

In some embodiments, a first subdomain comprises one or more modified nucleobases.

In some embodiments, a first subdomain comprise a nucleoside opposite to a target adenosine, e.g., when the oligonucleotide forms a duplex with a target nucleic acid. Suitable nucleobases including modified nucleobases in opposite nucleosides are described herein. For example, in some embodiment, an opposite nucleobase is optionally substituted or protected nucleobase selected from C, a tautomer of C, U, a tautomer of U, A, a tautomer of A, and a nucleobase which is or comprises Ring BA having the structure of BA-I, BA-I-a, BA-I-b, BA-II, BA-II-a, BA-II-b, BA-III, BA-III-a, BA-III-b, BA-IV, BA-IV-a, BA-IV-b, BA-V, BA-V-a, BA-V-b, or BA-VI, or a tautomer of Ring BA.

In some embodiments, a first subdomain comprises one or more sugars comprising two 2′-H (e.g., natural DNA sugars). In some embodiments, a first subdomain comprises one or more sugars comprising 2′-OH (e.g., natural RNA sugars). In some embodiments, a first subdomain comprises one or more modified sugars. In some embodiments, a modified sugar comprises a 2′-modification. In some embodiments, a modified sugar is a bicyclic sugar, e.g., a LNA sugar. In some embodiments, a modified sugar is an acyclic sugar (e.g., by breaking a C2-C3 bond of a corresponding cyclic sugar).

In some embodiments, a first subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars. In some embodiments, a first subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently bicyclic sugars (e.g., a LNA sugar) or a 2′-OR modified sugars, wherein R is independently optionally substituted C₁₋₆ aliphatic. In some embodiments, a first subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently 2′-OR modified sugars, wherein R is independently optionally substituted C₁₋₆ aliphatic. In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15. In some embodiments, the number is 16. In some embodiments, the number is 17. In some embodiments, the number is 18. In some embodiments, the number is 19. In some embodiments, the number is 20. In some embodiments, R is methyl.

In some embodiments, about 5%-100%, (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a first subdomain are independently a modified sugar. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a first subdomain are independently a bicyclic sugar (e.g., a LNA sugar) or a 2′-OR modified sugar, wherein R is independently optionally substituted C₁₋₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a first subdomain are independently a 2′-OR modified sugar, wherein R is independently optionally substituted C₁₋₆ aliphatic. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, R is methyl.

In some embodiments, a first subdomain comprises about 1-50 (e.g., about 5, 6, 7, 8, 9, or 10 about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a first subdomain are independently modified sugars with a modification that is not 2′-F. In some embodiments, about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in a first subdomain are independently modified sugars with a modification that is not 2′-F. In some embodiments, modified sugars of a first subdomain are each independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, a first subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a first subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic. In some embodiments, about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in a first subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, each sugar in a first subdomain independently comprises a 2′-OR modification, wherein R is optionally substituted C₁₋₆ aliphatic, or a 2′-O-L^{B}-4′ modification. In some embodiments, each sugar in a first subdomain independently comprises a 2′-OR modification, wherein R is optionally substituted C₁₋₆ aliphatic, or a 2′-O-L^{B}-4′ modification, wherein L^{B} is optionally substituted —CH₂—. In some embodiments, each sugar in a first subdomain independently comprises 2′-OMe.

In some embodiments, a first subdomain comprises one or more 2′-F modified sugars. In some embodiments, a first subdomain comprises no 2′-F modified sugars. In some embodiments, a first subdomain comprises one or more bicyclic sugars and/or 2′-OR modified sugars wherein R is not —H. In some embodiments, levels of bicyclic sugars and/or 2′-OR modified sugars wherein R is not —H, individually or combined, are relatively high compared to level of 2′-F modified sugars. In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a first subdomain comprises 2′-F. In some embodiments, no more than about 50% of sugars in a first subdomain comprises 2′-F. In some embodiments, a first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification. In some embodiments, a first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification. In some embodiments, a first subdomain comprises one or more (e.g., about 1-20, 1, 2, 3, 4, 5, 6, 7, 8, 9, 10, 11, 12, 13, 14, 15, 16, 17, 18, 19, 20, etc.) bicyclic sugars, e.g., LNA sugars. In some embodiments, a first subdomain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).

In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a first subdomain comprises 2′-MOE. In some embodiments, no more than about 50% of sugars in a first subdomain comprises 2′-MOE. In some embodiments, no sugars in a first subdomain comprises 2′-MOE.

In some embodiments, a first subdomain comprise about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in a first subdomain are modified internucleotidic linkages. In some embodiments, each internucleotidic linkage in a first subdomain is independently a modified internucleotidic linkage. In some embodiments, each modified internucleotidic linkages is independently a chiral internucleotidic linkage. In some embodiments, a modified or chiral internucleotidic linkage is a phosphorothioate internucleotidic linkage. In some embodiments, a modified or chiral internucleotidic linkage is a non-negatively charged internucleotidic linkage. In some embodiments, a modified or chiral internucleotidic linkage is a neutral internucleotidic linkage, e.g., n001. In some embodiments, each modified internucleotidic linkages is independently a phosphorothioate internucleotidic linkage or a non-negatively charged internucleotidic linkage. In some embodiments, each modified internucleotidic linkages is independently a phosphorothioate internucleotidic linkage or a neutral internucleotidic linkage. In some embodiments, each modified internucleotidic linkages is independently a phosphorothioate internucleotidic linkage. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a first subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a first subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a first subdomain is chirally controlled. In some embodiments, each is independently chirally controlled. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a first subdomain is Sp. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) phosphorothioate internucleotidic linkages in a first subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a first subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a first subdomain is Sp. In some embodiments, the number is one or more. In some embodiments, the number is 2 or more. In some embodiments, the number is 3 or more. In some embodiments, the number is 4 or more. In some embodiments, the number is 5 or more. In some embodiments, the number is 6 or more. In some embodiments, the number is 7 or more. In some embodiments, the number is 8 or more. In some embodiments, the number is 9 or more. In some embodiments, the number is 10 or more. In some embodiments, the number is 11 or more. In some embodiments, the number is 12 or more. In some embodiments, the number is 13 or more. In some embodiments, the number is 14 or more. In some embodiments, the number is 15 or more. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, each internucleotidic linkage linking two first subdomain nucleosides is independently a modified internucleotidic linkage. In some embodiments, each modified internucleotidic linkages is independently a chiral internucleotidic linkage. In some embodiments, each modified internucleotidic linkages is independently a phosphorothioate internucleotidic linkage. In some embodiments, each chiral internucleotidic linkage is independently a phosphorothioate internucleotidic linkage. In some embodiments, each modified internucleotidic linkages is independently a Sp chiral internucleotidic linkage. In some embodiments, each modified internucleotidic linkages is independently a Sp phosphorothioate internucleotidic linkage. In some embodiments, each chiral internucleotidic linkages is independently a Sp phosphorothioate internucleotidic linkage. In some embodiments, an internucleotidic linkage of a first subdomain is bonded to two nucleosides of the first subdomain. In some embodiments, an internucleotidic linkage bonded to a nucleoside in a first subdomain and a nucleoside in a second subdomain may be properly considered an internucleotidic linkage of a first subdomain. In some embodiments, an internucleotidic linkage bonded to a nucleoside in a first subdomain and a nucleoside in a second subdomain is a modified internucleotidic linkage; in some embodiments, it is a chiral internucleotidic linkage; in some embodiments, it is chirally controlled; in some embodiments, it is Rp; in some embodiments, it is Sp.

In some embodiments, a first subdomain comprises a certain level of Rp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in a first subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in a first subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in a first subdomain. In some embodiments, a percentage is about or no more than about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, about 1-50, 1-40, 1-30, 1-25, 1-20, 1-15, 1-10, 1-5, e.g., about 1, 2, 3, 4, 5, 6, 7, 8, 9, 10, 11, 12, 13, 14, 15, 16, 17, 18, 19, or 20 internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10.

In some embodiments, each phosphorothioate internucleotidic linkage in a first subdomain is independently chirally controlled. In some embodiments, each is independently Sp or Rp. In some embodiments, a high level is Sp as described herein. In some embodiments, each phosphorothioate internucleotidic linkage in a first subdomain is chirally controlled and is Sp. In some embodiments, one or more, e.g., about 1-5 (e.g., about 1, 2, 3, 4, or 5) is Rp.

In some embodiments, as illustrated in certain examples, a first subdomain comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in a first subdomain is about 1-10, or about 1, 2, 3, 4, 5, 6, 7, 8, 9, or 10. In some embodiments, it is about 1. In some embodiments, it is about 2. In some embodiments, it is about 3. In some embodiments, it is about 4. In some embodiments, it is about 5. In some embodiments, two or more non-negatively charged internucleotidic linkages are consecutive. In some embodiments, no two non-negatively charged internucleotidic linkages are consecutive. In some embodiments, all non-negatively charged internucleotidic linkages in a first subdomain are consecutive (e.g., 3 consecutive non-negatively charged internucleotidic linkages). In some embodiments, a non-negatively charged internucleotidic linkage, or two or more (e.g., about 2, about 3, about 4 etc.) consecutive non-negatively charged internucleotidic linkages, are at the 3′-end of a first subdomain. In some embodiments, the last two or three or four internucleotidic linkages of a first subdomain comprise at least one internucleotidic linkage that is not a non-negatively charged internucleotidic linkage. In some embodiments, the last two or three or four internucleotidic linkages of a first subdomain comprise at least one internucleotidic linkage that is not n001. In some embodiments, the internucleotidic linkage linking the last two nucleosides of a first subdomain is a non-negatively charged internucleotidic linkage. In some embodiments, the internucleotidic linkage linking the last two nucleosides of a first subdomain is a Sp non-negatively charged internucleotidic linkage. In some embodiments, the internucleotidic linkage linking the last two nucleosides of a first subdomain is a Rp non-negatively charged internucleotidic linkage. In some embodiments, the internucleotidic linkage linking the last two nucleosides of a first subdomain is a phosphorothioate internucleotidic linkage. In some embodiments, the internucleotidic linkage linking the last two nucleosides of a first subdomain is a Sp phosphorothioate internucleotidic linkage. In some embodiments, the internucleotidic linkage linking the first two nucleosides of a first subdomain is a non-negatively charged internucleotidic linkage. In some embodiments, the internucleotidic linkage linking the first two nucleosides of a first subdomain is a Sp non-negatively charged internucleotidic linkage. In some embodiments, the internucleotidic linkage linking the first two nucleosides of a first subdomain is a Rp non-negatively charged internucleotidic linkage. In some embodiments, the internucleotidic linkage linking the first two nucleosides of a first subdomain is a phosphorothioate internucleotidic linkage. In some embodiments, the internucleotidic linkage linking the first two nucleosides of a first subdomain is a Sp phosphorothioate internucleotidic linkage. In some embodiments, a non-negatively charged internucleotidic linkage is a neutral internucleotidic linkage such as n001.

In some embodiments, a first subdomain comprises one or more natural phosphate linkages. In some embodiments, a first subdomain contains no natural phosphate linkages.

In some embodiments, a first subdomain comprises a 5′-end portion, e.g., one having a length of about 1-20, 1-15, 1-10, 3-8, or about 1, 2, 3, 4, 5, 6, 7, 8, 9, or 10 nucleobases. In some embodiments, a 5′-end portion has a length of about 3-6 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases. In some embodiments, a length is 6 nucleobases. In some embodiments, a length is 7 nucleobases. In some embodiments, a length is 8 nucleobases. In some embodiments, a length is 9 nucleobases. In some embodiments, a length is 10 nucleobases. In some embodiments, a 5′-end portion comprises the 5′-end nucleobase of a first subdomain.

In some embodiments, a 5′-end portion comprises one or more sugars having two 2′-H (e.g., natural DNA sugars). In some embodiments, a 5′-end portion comprises one or more sugars having 2′-OH (e.g., natural RNA sugars). In some embodiments, one or more (e.g., about 1-20, 1-15, 1-10, 3-8, or about 1, 2, 3, 4, 5, 6, 7, 8, 9, or 10) of sugars in a 5′-end portion are independently modified sugars. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 5′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, one or more of the modified sugars independently comprises 2′-F or 2′—OR, wherein R is independently optionally substituted C₁₋₆ aliphatic. In some embodiments, one or more of the modified sugars are independently 2′-F or 2′-OMe. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a sugar with a 2′-OR modification wherein R is optionally substituted C₁₋₆ aliphatic. In some embodiments, R is methyl.

In some embodiments, one or more (e.g., about 1-10, or about 1, 2, 3, 4, 5, 6, 7, 8, 9, or 10) internucleotidic linkages of a 5′-end portion are independently a modified internucleotidic linkage. In some embodiments, one or more (e.g., about 1-10, or about 1, 2, 3, 4, 5, 6, 7, 8, 9, or 10) internucleotidic linkages of a 5′-end portion are independently a chiral internucleotidic linkage. In some embodiments, one or more (e.g., about 1-10, or about 1, 2, 3, 4, 5, 6, 7, 8, 9, or 10) internucleotidic linkages of a 5′-end portion are independently a chirally controlled internucleotidic linkage. In some embodiments, one or more (e.g., about 1-10, or about 1, 2, 3, 4, 5, 6, 7, 8, 9, or 10) internucleotidic linkages of a 5′-end portion are Rp. In some embodiments, one or more (e.g., about 1-10, or about 1, 2, 3, 4, 5, 6, 7, 8, 9, or 10) internucleotidic linkages of a 5′-end portion are Sp. In some embodiments, each internucleotidic linkage of a 5′-end portion is Sp.

In some embodiments, a 5′-end portion comprises one or more (e.g., about 1-10, or about 1, 2, 3, 4, 5, 6, 7, 8, 9, or 10) mismatches as described herein. In some embodiments, a 5′-end portion comprises one or more (e.g., about 1-10, or about 1, 2, 3, 4, 5, 6, 7, 8, 9, or 10) wobbles as described herein. In some embodiments, a 5′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a first subdomain comprises a 3′-end portion, e.g., one having a length of about 1-20, 1-15, 1-10, 1-5, 1-3, 3-8, or about 1, 2, 3, 4, 5, 6, 7, 8, 9, or 10 nucleobases. In some embodiments, a 3′-end portion has a length of about 1-3 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases. In some embodiments, a length is 6 nucleobases. In some embodiments, a length is 7 nucleobases. In some embodiments, a length is 8 nucleobases. In some embodiments, a length is 9 nucleobases. In some embodiments, a length is 10 nucleobases. In some embodiments, a 3′-end portion comprises the 3′-end nucleobase of a first subdomain. In some embodiments, a first subdomain comprises or consists of a 5′-end portion and a 3′-end portion.

In some embodiments, a 5′-end portion comprises one or more sugars having two 2′-H (e.g., natural DNA sugars). In some embodiments, a 5′-end portion comprises one or more sugars having 2′-OH (e.g., natural RNA sugars). In some embodiments, one or more (e.g., about 1-20, 1-15, 1-10, 3-8, or about 1, 2, 3, 4, 5, 6, 7, 8, 9, or 10) of sugars in a 3′-end portion are independently modified sugars. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 3′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, one or more of the modified sugars independently comprises 2′-F or 2′-OR, wherein R is independently optionally substituted C₁₋₆ aliphatic. In some embodiments, one or more of the modified sugars are independently 2′-F or 2′-OMe. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a sugar with a 2′-OR modification wherein R is optionally substituted C₁₋₆ aliphatic. In some embodiments, R is methyl.

In some embodiments, compared to a 5′-end portion, a 3′-end portion contains a higher level (in numbers and/or percentage) of 2′-F modified sugars and/or sugars comprising two 2′-H (e.g., natural DNA sugars), and/or a lower level (in numbers and/or percentage) of other types of modified sugars, e.g., bicyclic sugars and/or sugars with 2′-OR modifications wherein R is independently optionally substituted C₁-6 aliphatic. In some embodiments, compared to a 5′-end portion, a 3′-end portion contains a higher level of 2′-F modified sugars and/or a lower level of 2′-OR modified sugars wherein R is optionally substituted C₁₋₆ aliphatic. In some embodiments, compared to a 5′-end portion, a 3′-end portion contains a higher level of 2′-F modified sugars and/or a lower level of 2′-OMe modified sugars. In some embodiments, compared to a 5′-end portion, a 3′-end portion contains a higher level of natural DNA sugars and/or a lower level of 2′-OR modified sugars wherein R is optionally substituted C₁₋₆ aliphatic. In some embodiments, compared to a 5′-end portion, a 3′-end portion contains a higher level of natural DNA sugars and/or a lower level of 2′-OMe modified sugars. In some embodiments, a 3′-end portion contains low levels (e.g., no more than 50%, 40%, 30%, 25%, 20%, or 10%, or no more than 1, 2, 3, 4, 5, 6, 7, 8, 9, or 10) of modified sugars which are bicyclic sugars or sugars comprising 2′-OR wherein R is optionally substituted C₁₋₆ aliphatic (e.g., methyl). In some embodiments, a 3′-end portion contains no modified sugars which are bicyclic sugars or sugars comprising 2′-OR wherein R is optionally substituted C₁₋₆ aliphatic (e.g., methyl).

In some embodiments, one or more modified sugars independently comprise 2′-F. In some embodiments, no modified sugars comprises 2′-OMe or other 2′-OR modifications wherein R is optionally substituted C₁₋₆ aliphatic. In some embodiments, each sugar of a 3′-end portion independently comprises two 2′-H or a 2′-F modification. In some embodiments, a 3′-end portion comprises 1, 2, 3, 4, or 5 2′—F modified sugars. In some embodiments, a 3′-end portion comprises 1-3 2′—F modified sugars. In some embodiments, a 3′-end portion comprises 1, 2, 3, 4, or 5 natural DNA sugars. In some embodiments, a 3′-end portion comprises 1-3 natural DNA sugars.

In some embodiments, one or more (e.g., about 1-10, or about 1, 2, 3, 4, 5, 6, 7, 8, 9, or 10) internucleotidic linkages of a 3′-end portion are independently a modified internucleotidic linkage. In some embodiments, one or more (e.g., about 1-10, or about 1, 2, 3, 4, 5, 6, 7, 8, 9, or 10) internucleotidic linkages of a 3′-end portion are independently a chiral internucleotidic linkage. In some embodiments, one or more (e.g., about 1-10, or about 1, 2, 3, 4, 5, 6, 7, 8, 9, or 10) internucleotidic linkages of a 3′-end portion are independently a chirally controlled internucleotidic linkage. In some embodiments, one or more (e.g., about 1-10, or about 1, 2, 3, 4, 5, 6, 7, 8, 9, or 10) internucleotidic linkages of a 3′-end portion are Rp. In some embodiments, one or more (e.g., about 1-10, or about 1, 2, 3, 4, 5, 6, 7, 8, 9, or 10) internucleotidic linkages of a 3′-end portion are Sp. In some embodiments, each internucleotidic linkage of a 3′-end portion is Sp. In some embodiments, a 3′-end portion contains a higher level (in number and/or percentage) of Rp internucleotidic linkage and/or natural phosphate linkage compared to a 5′-end portion.

In some embodiments, a 3′-end portion comprises one or more (e.g., about 1-10, or about 1, 2, 3, 4, 5, 6, 7, 8, 9, or 10) mismatches as described herein. In some embodiments, a 3′-end portion comprises one or more (e.g., about 1-10, or about 1, 2, 3, 4, 5, 6, 7, 8, 9, or 10) wobbles as described herein. In some embodiments, a 3′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a first subdomain recruits, promotes or contribute to recruitment of, a protein such as an ADAR protein, e.g., ADAR1, ADAR2, etc. In some embodiments, a first subdomain recruits, or promotes or contribute to interactions with, a protein such as an ADAR protein. In some embodiments, a first subdomain contacts with a RNA binding domain (RBD) of ADAR. In some embodiments, a first subdomain contacts with a catalytic domain of ADAR which has a deaminase activity. In some embodiments, a first subdomain contact with a domain that has a deaminase activity of ADAR1. In some embodiments, a first subdomain contact with a domain that has a deaminase activity of ADAR2. In some embodiments, various nucleobases, sugars and/or internucleotidic linkages of a first subdomain may interact with one or more residues of proteins, e.g., ADAR proteins.

Second Subdomains

As described herein, in some embodiment, an oligonucleotide comprises a first domain and a second domain from 5′ to 3′. In some embodiments, a second domain comprises or consists of a first subdomain, a second subdomain, and a third subdomain from 5′ to 3′. Certain embodiments of a second subdomain are described below as examples. In some embodiments, a second subdomain comprise a nucleoside opposite to a target adenosine to be modified (e.g., conversion to I). In some embodiments, a second subdomain comprises one and no more than one nucleoside opposite to a target adenosine. In some embodiments, each nucleoside opposite to a target adenosine of an oligonucleotide is in a second subdomain.

In some embodiments, a second subdomain has a length of about 1-10, 1-5, 1-3, or about 1, 2, 3, 4, 5, 6, 7, 8, 9, or 10 nucleobases. In some embodiments, a second subdomain has a length of about 1-10 nucleobases. In some embodiments, a second subdomain has a length of about 1-5 nucleobases. In some embodiments, a second subdomain has a length of about 1-3 nucleobases. In some embodiments, a second subdomain has a length of 1 nucleobase. In some embodiments, a second subdomain has a length of 2 nucleobases. In some embodiments, a second subdomain has a length of 3 nucleobases.

In some embodiments, one or more (e.g., 1-20, 1, 2, 3, 4, 5, 6, 7, 8, 9, or 10, etc.) mismatches exist in a second subdomain when an oligonucleotide is aligned with a target nucleic acid for complementarity. In some embodiments, there is 1 mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches.

In some embodiments, a second subdomain comprises one and no more than one mismatch. In some embodiments, a second subdomain comprises two and no more than two mismatches. In some embodiments, a second subdomain comprises two and no more than two mismatches, wherein one mismatch is between a target adenosine and its opposite nucleoside, and/or one mismatch is between a nucleoside next to a target adenosine and its corresponding nucleoside in an oligonucleotide. In some embodiments, a mismatch between a nucleoside next to a target adenosine and its corresponding nucleoside in an oligonucleotide is a wobble. In some embodiments, a wobble is I-C. In some embodiments, C is next to a target adenosine, e.g., immediately to its 3′ side.

In some embodiments, one or more (e.g., 1-20, 1, 2, 3, 4, 5, 6, 7, 8, 9, or 10, etc.) wobbles exist in a second subdomain when an oligonucleotide is aligned with a target nucleic acid for complementarity. In some embodiments, there is 1 wobble. In some embodiments, there are 2 wobbles. In some embodiments, there are 3 wobbles. In some embodiments, there are 4 wobbles. In some embodiments, there are 5 wobbles. In some embodiments, there are 6 wobbles. In some embodiments, there are 7 wobbles. In some embodiments, there are 8 wobbles. In some embodiments, there are 9 wobbles. In some embodiments, there are 10 wobbles.

In some embodiments, duplexes of oligonucleotides and target nucleic acids in a second subdomain region comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, a second subdomain is fully complementary to a target nucleic acid.

In some embodiments, a second subdomain comprises one or more modified nucleobases.

In some embodiments, a second subdomain comprise a nucleoside opposite to a target adenosine, e.g., when the oligonucleotide forms a duplex with a target nucleic acid. Suitable nucleobases including modified nucleobases in opposite nucleosides are described herein. For example, in some embodiment, an opposite nucleobase is optionally substituted or protected nucleobase selected from C, a tautomer of C, U, a tautomer of U, A, a tautomer of A, and a nucleobase which is or comprises Ring BA having the structure of BA-I, BA-I-a, BA-I-b, BA-II, BA-II-a, BA-II-b, BA-III, BA-III-a, BA-III-b, BA-IV, BA-IV-a, BA-IV-b, BA-V, BA-V-a, BA-V-b, or BA-VI, or a tautomer of Ring BA. For example, in some embodiments, an opposite nucleobase is selected from

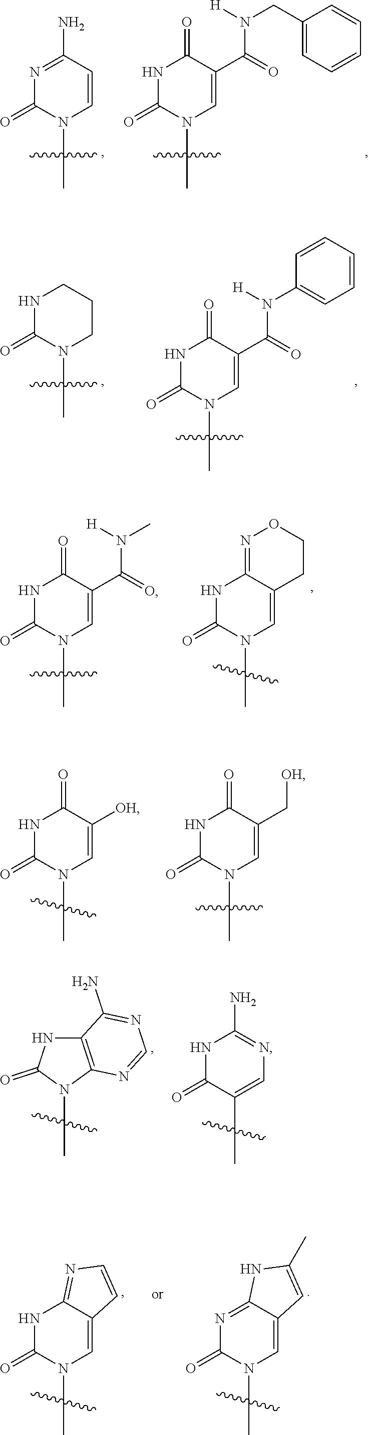

In some embodiments, an opposite nucleobase is

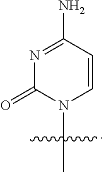 [SMILES: *n1ccc(N)nc1=O]

In some embodiments, an opposite nucleobase is

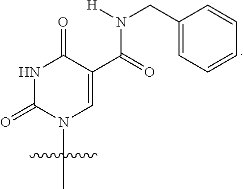 [SMILES: *n1cc(C(=O)NCc2ccccc2)c(=O)[nH]c1=O]

In some embodiments, an opposite nucleobase is

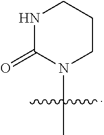 [SMILES: *N1CCCNC1=O]

In some embodiments, an opposite nucleobase is

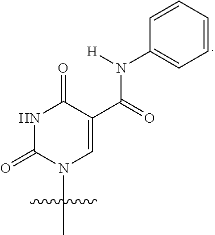 [SMILES: *n1cc(C(=O)Nc2ccccc2)c(=O)[nH]c1=O]

In some embodiments, an opposite nucleobase is

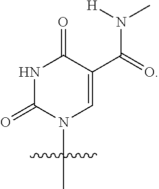 [SMILES: *n1cc(C(=O)NC)c(=O)[nH]c1=O]

In some embodiments, an opposite nucleobase is

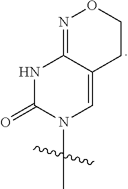 [SMILES: *N1C=C2CCON=C2NC1=O]

In some embodiments, an opposite nucleobase is

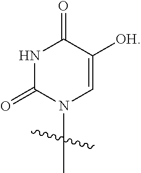 [SMILES: *n1cc(O)c(=O)[nH]c1=O]

In some embodiments, an opposite nucleobase is

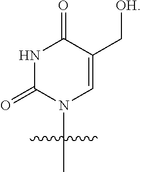 [SMILES: *n1cc(CO)c(=O)[nH]c1=O]

In some embodiments, an opposite nucleobase is

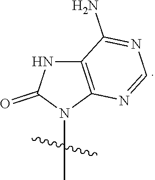 [SMILES: *n1c(=O)[nH]c2c(N)ncnc21]

In some embodiments, an opposite nucleobase is

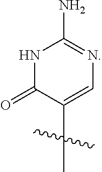 [SMILES: *c1cnc(N)[nH]c1=O]

In some embodiments, an opposite nucleobase is

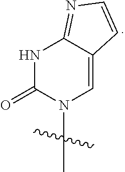 [SMILES: *n1cc2ccnc-2[nH]c1=O]

In some embodiments, an opposite nucleobase is or

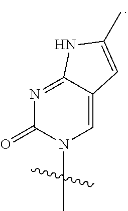 [SMILES: *n1cc2cc(C)[nH]c2nc1=O]

In some embodiments, a second subdomain comprises a modified nucleobase next to an opposite nucleobase. In some embodiments, it is to the 5′ side. In some embodiments, it is to the 3′ side. In some embodiments, on each side there is independently a modified nucleobase. Among other things, the present disclosure recognizes that nucleobases adjacent to (e.g., next to) opposite nucleobases may cause disruption (e.g., steric hindrance) to recognition, binding, interaction, and/or modification of target nucleic acids, oligonucleotides and/or duplexes thereof. In some embodiments, disruption is associated with an adjacent G. In some embodiments, the present disclosure provides nucleobases that can replace G and provide improved stability and/or activities compared to G. For example, in some embodiments, an adjacent nucleobase (e.g., 3′-immediate nucleoside of an opposite nucleoside) is hypoxanthine (replacing G to reduce disruption (e.g., steric hindrance) and/or forming wobble base pairing with C). In some embodiments, an adjacent nucleobase is a derivative of hypoxanthine. In some embodiments, 3′-immediate nucleoside comprises a nucleobase which is or comprise Ring BA having the structure of formula BA-VI. In some embodiments, an adjacent nucleobase is

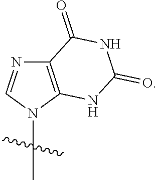 [SMILES: *n1cnc2c(=O)[nH]c(=O)[nH]c21]

In some embodiments, an adjacent nucleobase is

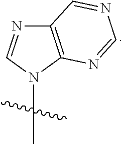 [SMILES: *n1cnc2cncnc21]

In some embodiments, a second subdomain comprises one or more sugars comprising two 2′-H (e.g., natural DNA sugars). In some embodiments, a second subdomain comprises one or more sugars comprising 2′-OH (e.g., natural RNA sugars). In some embodiments, a second subdomain comprises one or more modified sugars. In some embodiments, a modified sugar comprises a 2′-modification. In some embodiments, a modified sugar is a bicyclic sugar, e.g., a LNA sugar. In some embodiments, a modified sugar is an acyclic sugar (e.g., by breaking a C2-C3 bond of a corresponding cyclic sugar). In some embodiments, an opposite nucleoside comprises an acyclic sugar such as an UNA sugar. In some embodiments, such an acyclic sugar provides flexibility for proteins to perform modifications on a target adenosine.

In some embodiments, a second subdomain comprises about 1-10 (e.g., about 1, 2, 3, 4, 5, 6, 7, 8, 9, or 10) modified sugars independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a second subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, low levels (e.g., no more than 50%, 40%, 30%, 25%, 20%, or 10%, or no more than 1, 2, 3, 4, 5, 6, 7, 8, 9, or 10) of sugars in a second subdomain independently comprise a 2′-OR modification, wherein R is optionally substituted C₁₋₆ aliphatic, or a 2′-O-L^{B}-4′ modification. In some embodiments, each sugar in a second subdomain independently contains no 2′-OR modification, wherein R is optionally substituted C₁₋₆ aliphatic, or a 2′-O-L^{B}-4′ modification, wherein L^{B} is optionally substituted —CH₂—. In some embodiments, each sugar in a second subdomain independently contains no 2′-OMe.

In some embodiments, a second subdomain comprises one or more 2′-F modified sugars.

In some embodiments, a high level (e.g., about 60-100%, or about 60%, 65%, 70%, 75%, 80%, 85%, 90%, 95% or more, or 100%) or all sugars in a second subdomain are independently 2′-F modified sugars, sugars comprising two 2′-H (e.g., natural DNA sugars), or sugars comprising 2′-OH (e.g., natural RNA sugars). In some embodiments, a high level (e.g., about 60-100%, or about 60%, 65%, 70%, 75%, 80%, 85%, 90%, 95% or more, or 100%) or all sugars in a second subdomain are independently 2′-F modified sugars, natural DNA sugars, or natural RNA sugars. In some embodiments, a high level (e.g., about 60-100%, or about 60%, 65%, 70%, 75%, 80%, 85%, 90%, 95% or more, or 100%) or all sugars in a second subdomain are independently 2′-F modified sugars and natural DNA sugars. In some embodiments, a level is 100%. In some embodiments, a second subdomain comprise 1, 2, 3, 4 or 5 2′—F modified sugars. In some embodiments, a second subdomain comprise 1, 2, 3, 4 or 5 sugars comprising two 2′-H. In some embodiments, a second subdomain comprise 1, 2, 3, 4 or 5 natural DNA sugars. In some embodiments, a second subdomain comprise 1, 2, 3, 4 or 5 sugars comprising 2′-OH. In some embodiments, a second subdomain comprise 1, 2, 3, 4 or 5 natural RNA sugars. In some embodiments, a number is 1. In some embodiments, a number is 2. In some embodiments, a number is 3. In some embodiments, a number is 4. In some embodiments, a number is 5.

In some embodiments, sugars of opposite nucleosides to target adenosines (“opposite sugars”), sugars of nucleosides 5′-next to opposite nucleosides (“5′-next sugars”), and/or sugars of nucleosides 3′-next to opposite nucleosides (“3-next sugars”) are independently and optionally 2′-F modified sugars, sugars comprising two 2′-H (e.g., natural DNA sugars), or sugars comprising 2′-OH (e.g., natural RNA sugars). In some embodiments, an opposite sugar is a 2′-F modified sugar. In some embodiments, an opposite sugar is a sugar comprising two 2′-H. In some embodiments, an opposite sugar is a natural DNA sugar. In some embodiments, an opposite sugar is a sugar comprising 2′-OH. In some embodiments, an opposite sugar is a natural RNA sugar. For example, in some embodiments, each of a 5′-next sugar, an opposite sugar and a 3′-next sugar in an oligonucleotide is independently a natural DNA sugar. In some embodiments, a 5′-next sugar is a 2′-F modified sugar, and each of an opposite sugar and a 3′-next sugar is independently a natural DNA sugar.

In some embodiments, a 5′-next sugar is a 2′-F modified sugar. In some embodiments, a 5′-next sugar is a sugar comprising two 2′-H. In some embodiments, a 5′-next sugar is a natural DNA sugar. In some embodiments, a 5′-next sugar is a sugar comprising 2′-OH. In some embodiments, a 5′-next sugar is a natural RNA sugar.

In some embodiments, a 3′-next sugar is a 2′-F modified sugar. In some embodiments, a 3′-next sugar is a sugar comprising two 2′-H. In some embodiments, a 3′-next sugar is a natural DNA sugar. In some embodiments, a 3′-next sugar is a sugar comprising 2′-OH. In some embodiments, a 3′-next sugar is a natural RNA sugar.

In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a second subdomain comprises 2′-MOE. In some embodiments, no more than about 50% of sugars in a second subdomain comprises 2′-MOE. In some embodiments, no sugars in a second subdomain comprises 2′-MOE.

In some embodiments, a second subdomain comprise about 1-10 (e.g., about 1-5, 1-4, 1-3, about 1, 2, 3, 4, 5, 6, 7, 8, 9, or 10) modified internucleotidic linkages. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in a second subdomain are modified internucleotidic linkages. In some embodiments, each internucleotidic linkage in a second subdomain is independently a modified internucleotidic linkage. In some embodiments, each modified internucleotidic linkages is independently a chiral internucleotidic linkage. In some embodiments, a modified or chiral internucleotidic linkage is a phosphorothioate internucleotidic linkage. In some embodiments, a modified or chiral internucleotidic linkage is a non-negatively charged internucleotidic linkage. In some embodiments, a modified or chiral internucleotidic linkage is a neutral internucleotidic linkage, e.g., n001. In some embodiments, each modified internucleotidic linkages is independently a phosphorothioate internucleotidic linkage or a non-negatively charged internucleotidic linkage. In some embodiments, each modified internucleotidic linkages is independently a phosphorothioate internucleotidic linkage or a neutral internucleotidic linkage. In some embodiments, each modified internucleotidic linkages is independently a phosphorothioate internucleotidic linkage. In some embodiments, at least about 1-10 (e.g., about 1-5, 1-4, 1-3, about 1, 2, 3, 4, 5, 6, 7, 8, 9, or 10) chiral internucleotidic linkages in a second subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a second subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a second subdomain is chirally controlled. In some embodiments, each is independently chirally controlled. In some embodiments, at least about 1-10 (e.g., about 1-5, 1-4, 1-3, about 1, 2, 3, 4, 5, 6, 7, 8, 9, or 10) chiral internucleotidic linkages in a second subdomain is Sp. In some embodiments, at least about 1-10 (e.g., about 1-5, 1-4, 1-3, about 1, 2, 3, 4, 5, 6, 7, 8, 9, or 10) phosphorothioate internucleotidic linkages in a second subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a second subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a second subdomain is Sp. In some embodiments, the number is one or more. In some embodiments, the number is 2 or more. In some embodiments, the number is 3 or more. In some embodiments, the number is 4 or more. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, each internucleotidic linkage linking two second subdomain nucleosides is independently a modified internucleotidic linkage. In some embodiments, each modified internucleotidic linkages is independently a chiral internucleotidic linkage. In some embodiments, each modified internucleotidic linkages is independently a phosphorothioate internucleotidic linkage. In some embodiments, each chiral internucleotidic linkage is independently a phosphorothioate internucleotidic linkage. In some embodiments, each modified internucleotidic linkages is independently a Sp chiral internucleotidic linkage. In some embodiments, each modified internucleotidic linkages is independently a Sp phosphorothioate internucleotidic linkage. In some embodiments, each chiral internucleotidic linkages is independently a Sp phosphorothioate internucleotidic linkage. In some embodiments, an internucleotidic linkage of a second subdomain is bonded to two nucleosides of the second subdomain. In some embodiments, an internucleotidic linkage bonded to a nucleoside in a second subdomain and a nucleoside in a first or third subdomain may be properly considered an internucleotidic linkage of a second subdomain. In some embodiments, an internucleotidic linkage bonded to a nucleoside in a second subdomain and a nucleoside in a first or third subdomain is a modified internucleotidic linkage; in some embodiments, it is a chiral internucleotidic linkage; in some embodiments, it is chirally controlled; in some embodiments, it is Rp; in some embodiments, it is Sp.

In some embodiments, a second subdomain comprises a certain level of Rp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in a second subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in a second subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in a second subdomain. In some embodiments, a percentage is about or no more than about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, 1-10 (e.g., about 1-5, 1-4, 1-3, about 1, 2, 3, 4, 5, 6, 7, 8, 9, or 10) internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10. In some embodiments, a second subdomain comprise a higher level (in number and/or percentage) of Rp internucleotidic linkage compared to other portions (e.g., a first domain, a second domain overall, a first subdomain, a third subdomain, or portions thereof). In some embodiments, a second subdomain comprise a higher level (in number and/or percentage) of Rp internucleotidic linkage than Sp internucleotidic linkage.

In some embodiments, each phosphorothioate internucleotidic linkage in a second subdomain is independently chirally controlled. In some embodiments, each is independently Sp or Rp. In some embodiments, a high level is Sp as described herein. In some embodiments, each phosphorothioate internucleotidic linkage in a second subdomain is chirally controlled and is Sp. In some embodiments, one or more, e.g., about 1-5 (e.g., about 1, 2, 3, 4, or 5) is Rp.

In some embodiments, as illustrated in certain examples, a second subdomain comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in a second subdomain is about 1-5, or about 1, 2, 3, 4, or 5. In some embodiments, it is about 1. In some embodiments, it is about 2. In some embodiments, it is about 3. In some embodiments, it is about 4. In some embodiments, it is about 5. In some embodiments, two or more non-negatively charged internucleotidic linkages are consecutive. In some embodiments, no two non-negatively charged internucleotidic linkages are consecutive. In some embodiments, all non-negatively charged internucleotidic linkages in a second subdomain are consecutive (e.g., 3 consecutive non-negatively charged internucleotidic linkages). In some embodiments, a non-negatively charged internucleotidic linkage, or two or more (e.g., about 2, about 3, about 4 etc.) consecutive non-negatively charged internucleotidic linkages, are at the 3′-end of a second subdomain. In some embodiments, the last two or three or four internucleotidic linkages of a second subdomain comprise at least one internucleotidic linkage that is not a non-negatively charged internucleotidic linkage. In some embodiments, the last two or three or four internucleotidic linkages of a second subdomain comprise at least one internucleotidic linkage that is not n001. In some embodiments, the internucleotidic linkage linking the last two nucleosides of a second subdomain is a non-negatively charged internucleotidic linkage. In some embodiments, the internucleotidic linkage linking the last two nucleosides of a second subdomain is a Sp non-negatively charged internucleotidic linkage. In some embodiments, the internucleotidic linkage linking the last two nucleosides of a second subdomain is a Rp non-negatively charged internucleotidic linkage. In some embodiments, the internucleotidic linkage linking the last two nucleosides of a second subdomain is a phosphorothioate internucleotidic linkage. In some embodiments, the internucleotidic linkage linking the last two nucleosides of a second subdomain is a Sp phosphorothioate internucleotidic linkage. In some embodiments, the internucleotidic linkage linking the first two nucleosides of a second subdomain is a non-negatively charged internucleotidic linkage. In some embodiments, the internucleotidic linkage linking the first two nucleosides of a second subdomain is a Sp non-negatively charged internucleotidic linkage. In some embodiments, the internucleotidic linkage linking the first two nucleosides of a second subdomain is a Rp non-negatively charged internucleotidic linkage. In some embodiments, the internucleotidic linkage linking the first two nucleosides of a second subdomain is a phosphorothioate internucleotidic linkage. In some embodiments, the internucleotidic linkage linking the first two nucleosides of a second subdomain is a Sp phosphorothioate internucleotidic linkage. In some embodiments, the internucleotidic linkage linking the last nucleoside of a second subdomain and the first nucleoside of a third subdomain is a non-negatively charged internucleotidic linkage. In some embodiments, the internucleotidic linkage linking the last nucleoside of a second subdomain and the first nucleoside of a third subdomain is a Sp non-negatively charged internucleotidic linkage. In some embodiments, the internucleotidic linkage linking the last nucleoside of a second subdomain and the first nucleoside of a third subdomain is a Rp non-negatively charged internucleotidic linkage. In some embodiments, the internucleotidic linkage linking the last nucleoside of a second subdomain and the first nucleoside of a third subdomain is a phosphorothioate internucleotidic linkage. In some embodiments, the internucleotidic linkage linking the last nucleoside of a second subdomain and the first nucleoside of a third subdomain is a Sp phosphorothioate internucleotidic linkage. In some embodiments, a non-negatively charged internucleotidic linkage is a neutral internucleotidic linkage such as n001.

In some embodiments, a second subdomain comprises one or more natural phosphate linkages. In some embodiments, a second subdomain contains no natural phosphate linkages. In some embodiments, a second subdomain comprises at least 1 natural phosphate linkage. In some embodiments, a second subdomain comprises at least 2 natural phosphate linkages. In some embodiments, a second subdomain comprises at least 3 natural phosphate linkages. In some embodiments, a second subdomain comprises at least 4 natural phosphate linkages. In some embodiments, a second subdomain comprises at least 5 natural phosphate linkages.

In some embodiments, an opposite nucleoside is connected to its 5′ immediate nucleoside through a natural phosphate linkage. In some embodiments, an opposite nucleoside is connected to its 5′ immediate nucleoside through a natural phosphate linkage. In some embodiments, an opposite nucleoside is connected to its 5′ immediate nucleoside through a modified internucleotidic linkage. In some embodiments, a modified internucleotidic linkage is a chiral internucleotidic linkage. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral charged internucleotidic linkage. In some embodiments, a chiral internucleotidic linkage is chirally controlled. In some embodiments, a chiral internucleotidic linkage is Rp. In some embodiments, a chiral internucleotidic linkage is Sp.

In some embodiments, an opposite nucleoside is connected to its 3′ immediate nucleoside (−1 position relative to the opposite nucleoside) through a natural phosphate linkage. In some embodiments, an opposite nucleoside is connected to its 3′ immediate nucleoside through a modified internucleotidic linkage. In some embodiments, a modified internucleotidic linkage is a chiral internucleotidic linkage. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral charged internucleotidic linkage. In some embodiments, a chiral internucleotidic linkage is chirally controlled. In some embodiments, a chiral internucleotidic linkage is Rp. In some embodiments, a chiral internucleotidic linkage is Sp. In some embodiments, a chiral internucleotidic linkage is a phosphorothioate internucleotidic linkage and is chirally controlled. In some embodiments, a chiral internucleotidic linkage is a phosphorothioate internucleotidic linkage and is Sp. In some embodiments, a chiral internucleotidic linkage is a phosphorothioate internucleotidic linkage and is Rp. In some embodiments, a chiral internucleotidic linkage is a non-negatively charged internucleotidic linkage (e.g., n001) and is chirally controlled. In some embodiments, a chiral internucleotidic linkage is a non-negatively charged internucleotidic linkage (e.g., n001) and is chirally controlled and is Rp. In some embodiments, a chiral internucleotidic linkage is a non-negatively charged internucleotidic linkage (e.g., n001) and is chirally controlled and is Sp. In some embodiments, a chiral internucleotidic linkage is a non-negatively charged internucleotidic linkage (e.g., n001) and is not chirally controlled.

In some embodiments, a nucleoside at −1 position relative to an opposite nucleoside and a nucleoside at −2 position relative to an opposite nucleoside (e.g., in if No is an opposite nucleoside, N₋₁ is at −1 position and N₋₂ is at −2 position) is linked through a natural phosphate linkage. In some embodiments, they are connected through a modified internucleotidic linkage. In some embodiments, a modified internucleotidic linkage is a chiral internucleotidic linkage. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral charged internucleotidic linkage. In some embodiments, a chiral internucleotidic linkage is chirally controlled. In some embodiments, a chiral internucleotidic linkage is Rp. In some embodiments, a chiral internucleotidic linkage is Sp. In some embodiments, a chiral internucleotidic linkage is a phosphorothioate internucleotidic linkage and is chirally controlled. In some embodiments, a chiral internucleotidic linkage is a phosphorothioate internucleotidic linkage and is Sp. In some embodiments, a chiral internucleotidic linkage is a phosphorothioate internucleotidic linkage and is Rp. In some embodiments, a chiral internucleotidic linkage is a non-negatively charged internucleotidic linkage (e.g., n001) and is chirally controlled. In some embodiments, a chiral internucleotidic linkage is a non-negatively charged internucleotidic linkage (e.g., n001) and is chirally controlled and is Rp. In some embodiments, a chiral internucleotidic linkage is a non-negatively charged internucleotidic linkage (e.g., n001) and is chirally controlled and is Sp. In some embodiments, a chiral internucleotidic linkage is a non-negatively charged internucleotidic linkage (e.g., n001) and is not chirally controlled.

In some embodiments, a nucleoside of a second subdomain and a nucleoside of a third subdomain is linked through a natural phosphate linkage. In some embodiments, they are connected through a modified internucleotidic linkage. In some embodiments, a modified internucleotidic linkage is a chiral internucleotidic linkage. In some embodiments, a modified internucleotidic linkage is a phosphorothioate internucleotidic linkage. In some embodiments, a modified internucleotidic linkage is a non-negatively charged internucleotidic linkage. In some embodiments, a modified internucleotidic linkage is a neutral charged internucleotidic linkage. In some embodiments, a chiral internucleotidic linkage is chirally controlled. In some embodiments, a chiral internucleotidic linkage is Rp. In some embodiments, a chiral internucleotidic linkage is Sp. In some embodiments, a chiral internucleotidic linkage is a phosphorothioate internucleotidic linkage and is chirally controlled. In some embodiments, a chiral internucleotidic linkage is a phosphorothioate internucleotidic linkage and is Sp. In some embodiments, a chiral internucleotidic linkage is a phosphorothioate internucleotidic linkage and is Rp. In some embodiments, a chiral internucleotidic linkage is a non-negatively charged internucleotidic linkage (e.g., n001) and is chirally controlled. In some embodiments, a chiral internucleotidic linkage is a non-negatively charged internucleotidic linkage (e.g., n001) and is chirally controlled and is Rp. In some embodiments, a chiral internucleotidic linkage is a non-negatively charged internucleotidic linkage (e.g., n001) and is chirally controlled and is Sp. In some embodiments, a chiral internucleotidic linkage is a non-negatively charged internucleotidic linkage (e.g., n001) and is not chirally controlled.

In some embodiments, an oligonucleotide comprises 5′-N₁N₀N₋₁-3′, wherein each of N₁, N₀, and N₋₁ is independently a nucleoside, N₁ and N₀ bond to an internucleotidic linkage as described herein, and N₋₁ and No bond to an internucleotidic linkage as described herein, and No is opposite to a target adenosine. In some embodiments, the sugar of each of N₁, N₀, and N₋₁ is independently a natural DNA sugar or a 2′-F modified sugar. In some embodiments, the sugar of each of N₁, N₀, and N₋₁ is independently a natural DNA sugar. In some embodiments, the sugar of N₁ is a 2′-modified sugar, and the sugar of each of N₀ and N₋₁ is independently a natural DNA sugar. In some embodiments, such oligonucleotides provide high editing levels. In some embodiments, each of the two internucleotidic linkages bonded to N₋₁ is independently Rp. In some embodiments, each of the two internucleotidic linkages bonded to N₋₁ is independently an Rp phosphorothioate internucleotidic linkage. In some embodiments, each of the two internucleotidic linkages bonded to N₋₁ is independently an Rp phosphorothioate internucleotidic linkage, and each other phosphorothioate internucleotidic linkage in an oligonucleotide, if any, is independently Sp. In some embodiments, a 5′ internucleotidic linkage bonded to N₁ is Rp. In some embodiments, an internucleotidic linkage bonded to N₁ and N₀ (i.e., a 3′ internucleotidic linkage bonded to N₁) is Rp. In some embodiments, an internucleotidic linkage bonded to N₋₁ and N₀ is Rp. In some embodiments, a 3′ internucleotidic linkage bonded to N₋₁ is Rp. In some embodiments, each internucleotidic linkage bonded to N₀ is independently Rp. In some embodiments, each internucleotidic linkage bonded to N₀ or N₁ is independently Rp. In some embodiments, each internucleotidic linkage bonded to N₀ or N₋₁ is independently Rp. In some embodiments, each internucleotidic linkage bonded to N₁ is independently Rp. In some embodiments, each Rp internucleotidic linkage is independently an Rp phosphorothioate internucleotidic linkage. In some embodiments, each other chirally controlled phosphorothioate internucleotidic linkage in an oligonucleotide is independently Sp.

In some embodiments, sugar of a 5′ immediate nucleoside (e.g., N₁) is independently selected from a natural DNA sugar, a natural RNA sugar, and a 2′-F modified sugar (e.g., R^{2s} is —F). In some embodiments, sugar of an opposite nucleoside (e.g., N₀) is independently selected from a natural DNA sugar, a natural RNA sugar, and a 2′-F modified sugar. In some embodiments, sugar of a 3′ immediate nucleoside (e.g., N₋₁) is independently selected from a natural DNA sugar, a natural RNA sugar, and a 2′-F modified sugar. In some embodiments, sugars of a 5′ immediate nucleoside, an opposite nucleoside, and a 3′ immediate nucleoside are each independently a natural DNA sugar. In some embodiments, sugars of a 5′ immediate nucleoside, an opposite nucleoside, and a 3′ immediate nucleoside are a natural DNA sugar, a natural RNA sugar, and natural DNA sugar, respectively. In some embodiments, sugars of a 5′ immediate nucleoside, an opposite nucleoside, and a 3′ immediate nucleoside are a 2′-F modified sugar, a natural RNA sugar, and natural DNA sugar, respectively.

In some embodiments, sugar of an opposite nucleoside is a natural RNA sugar. In some embodiments, such an opposite nucleoside is utilized with a 3′ immediate I nucleoside (which is optionally complementary to a C in a target nucleic acid when aligned). In some embodiments, an internucleotidic linkage between the 3′ immediate nucleoside (e.g., N₋₁) and its 3′ immediate nucleoside (e.g., N_2) is a non-negatively charged internucleotidic linkage, e.g., n001. In some embodiments, it is stereorandom. In some embodiments, it is chirally controlled and is Rp. In some embodiments, it is chirally controlled and is Sp.

In some embodiments, an internucleotidic linkage that is bonded to a 3′ immediate nucleoside (e.g., N₋₁) and its 3′ neighboring nucleoside (e.g., N₋₂ in 5′-N₁N₀N₋₁N₋₂-3′) is a modified internucleotidic linkage. In some embodiments, it is a chiral internucleotidic linkage. In some embodiments, it is stereorandom. In some embodiments, it is a stereorandom phosphorothioate internucleotidic linkage. In some embodiments, it is a stereorandom non-negatively charged internucleotidic linkage. In some embodiments, it is stereorandom n001. In some embodiments, it is chirally controlled. In some embodiments, it is a Rp phosphorothioate internucleotidic linkage. In some embodiments, it is a Sp phosphorothioate internucleotidic linkage. In some embodiments, it is chirally controlled. In some embodiments, it is a Rp non-negatively charged internucleotidic linkage. In some embodiments, it is a Sp non-negatively charged internucleotidic linkage. In some embodiments, a non-negatively charged internucleotidic linkage is a neutral internucleotidic linkage. In some embodiments, a non-negatively charged internucleotidic linkage is n001.

In some embodiments, N₋₁ is I. In some embodiments, I is utilized replacing G, e.g., when a target nucleic acid comprises 5′—CA-3′ wherein A is a target adenosine. In some embodiments, 5′-N₁N₀N₋₁-3′ is 5′-N₁N₀I-3′. In some embodiments, N₀ is b001A, b002A, b003A, b008U, b001C, C, A, or U. In some embodiments, N₀ is b001A, b002A, b008U, b001C, C, or A. In some embodiments, N₀ is b001A, b002A, b008U, or b001C. In some embodiments, N₀ is b001A. In some embodiments, N₀ is b002A. In some embodiments, N₀ is b003A. In some embodiments, N₀ is b008U. In some embodiments, N₀ is b001C. In some embodiments, N₀ is A. In some embodiments, N₀ is U.

As demonstrated herein, in some embodiments provided oligonucleotides comprising certain nucleobases (e.g., b001A, b002A, b008U, C, A, etc.) opposite to target adenosines can among other things provide improved editing efficiency (e.g., compared to a reference nucleobase such as U). In some embodiments, an opposite nucleoside is linked to an I to its 3′ side.

In some embodiments, a second subdomain comprises a 5′-end portion, e.g., one having a length of about 1-5, 1-3, or 1, 2, 3, 4, or 5 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases.

In some embodiments, a 5′-end portion comprises one or more sugars having two 2′-H (e.g., natural DNA sugars). In some embodiments, a 5′-end portion comprises one or more sugars having 2′-OH (e.g., natural RNA sugars). In some embodiments, one or more (e.g., about 1-5, 1-3, or 1, 2, 3, 4, or 5) of sugars in a 5′-end portion are independently modified sugars. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 5′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, low levels (e.g., no more than 50%, 40%, 30%, 25%, 20%, or 10%, or no more than 1, 2, 3, 4, 5, 6, 7, 8, 9, or 10) of sugars in a 5′-end portion independently comprise a 2′-OR modification, wherein R is optionally substituted C₁₋₆ aliphatic, or a 2′-O-L^{B}-4′ modification. In some embodiments, each sugar in a 5′-end portion independently contains no 2′-OR modification, wherein R is optionally substituted C₁₋₆ aliphatic, or a 2′-O-L^{B}-4′ modification, wherein L^{B} is optionally substituted —CH₂—. In some embodiments, each sugar in a 5′-end portion independently contains no 2′-OMe.

In some embodiments, a 5′-end portion comprises one or more 2′-F modified sugars.

In some embodiments, a high level (e.g., about 60-100%, or about 60%, 65%, 70%, 75%, 80%, 85%, 90%, 95% or more, or 100%) or all sugars in a 5′-end are independently 2′-F modified sugars, sugars comprising two 2′-H (e.g., natural DNA sugars), or sugars comprising 2′-OH (e.g., natural RNA sugars). In some embodiments, a high level (e.g., about 60-100%, or about 60%, 65%, 70%, 75%, 80%, 85%, 90%, 95% or more, or 100%) or all sugars in a 5′-end portion are independently 2′-F modified sugars, natural DNA sugars, or natural RNA sugars. In some embodiments, a high level (e.g., about 60-100%, or about 60%, 65%, 70%, 75%, 80%, 85%, 90%, 95% or more, or 100%) or all sugars in a 5′-end portion are independently 2′-F modified sugars and natural DNA sugars. In some embodiments, a level is 100%. In some embodiments, sugars of a 5′-end portion are selected from sugars having two 2′-H (e.g., natural DNA sugar) and 2′-F modified sugars. In some embodiments, a 5′-end portion comprise 1, 2, 3, 4 or 5 2′—F modified sugars. In some embodiments, a 5′-end portion comprise 1, 2, 3, 4 or 5 sugars comprising two 2′-H. In some embodiments, a 5′-end portion comprise 1, 2, 3, 4 or 5 natural DNA sugars. In some embodiments, a 5′-end portion comprise 1, 2, 3, 4 or 5 sugars comprising 2′-OH. In some embodiments, a 5′-end portion comprise 1, 2, 3, 4 or 5 natural RNA sugars. In some embodiments, a number is 1. In some embodiments, a number is 2. In some embodiments, a number is 3. In some embodiments, a number is 4. In some embodiments, a number is 5.

In some embodiments, one or more (e.g., about 1, 2, 3, 4, or 5) internucleotidic linkages of a 5′-end portion are independently a modified internucleotidic linkage. In some embodiments, one or more (e.g., about 1, 2, 3, 4, or 5) internucleotidic linkages of a 5′-end portion are independently a chiral internucleotidic linkage. In some embodiments, one or more (e.g., about 1, 2, 3, 4, or 5) internucleotidic linkages of a 5′-end portion are independently a chirally controlled internucleotidic linkage. In some embodiments, one or more (e.g., about 1, 2, 3, 4, or 5) internucleotidic linkages of a 5′-end portion are Rp. In some embodiments, one or more (e.g., about 1, 2, 3, 4, or 5) internucleotidic linkages of a 5′-end portion are Sp. In some embodiments, each internucleotidic linkage of a 5′-end portion is Sp.

In some embodiments, one or more (e.g., about 1, 2, 3, 4, or 5) internucleotidic linkages of a 5′-end portion are independently a modified internucleotidic linkage. In some embodiments, one or more (e.g., about 1, 2, 3, 4, or 5) internucleotidic linkages of a 5′-end portion are independently a chiral internucleotidic linkage. In some embodiments, one or more (e.g., about 1, 2, 3, 4, or 5) internucleotidic linkages of a 5′-end portion are independently a chirally controlled internucleotidic linkage. In some embodiments, one or more (e.g., about 1, 2, 3, 4, or 5) internucleotidic linkages of a 5′-end portion are Rp. In some embodiments, one or more (e.g., about 1, 2, 3, 4, or 5) internucleotidic linkages of a 5′-end portion are Rp. In some embodiments, each internucleotidic linkage of a 5′-end portion is Rp.

In some embodiments, a 5′-end portion comprises one or more (e.g., about 1, 2, 3, 4, or 5) mismatches as described herein. In some embodiments, a 5′-end portion comprises one or more (e.g., about 1, 2, 3, 4, or 5) wobbles as described herein. In some embodiments, a 5′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a 5′-end portion comprises a nucleoside 5′ next to an opposite nucleoside. In some embodiments, a nucleoside 5′ next to an opposite nucleoside comprise a nucleobase as described herein.

In some embodiments, a second subdomain comprises a 3′-end portion, e.g., one having a length of about 1-5, 1-3, or 1, 2, 3, 4, or 5 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases. In some embodiments, a second subdomain consists a 5′-end portion and a 3′-end portion.

In some embodiments, a 3′-end portion comprises one or more sugars having two 2′-H (e.g., natural DNA sugars). In some embodiments, a 3′-end portion comprises one or more sugars having 2′-OH (e.g., natural RNA sugars). In some embodiments, one or more (e.g., about 1-5, 1-3, or 1, 2, 3, 4, or 5) of sugars in a 3′-end portion are independently modified sugars. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 3′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, low levels (e.g., no more than 50%, 40%, 30%, 25%, 20%, or 10%, or no more than 1, 2, 3, 4, 5, 6, 7, 8, 9, or 10) of sugars in a 3′-end portion independently comprise a 2′-OR modification, wherein R is optionally substituted C₁₋₆ aliphatic, or a 2′-O-L^{B}-4′ modification. In some embodiments, each sugar in a 3′-end portion independently contains no 2′-OR modification, wherein R is optionally substituted C₁₋₆ aliphatic, or a 2′-O-L^{B}-4′ modification, wherein L^{B} is optionally substituted —CH₂—. In some embodiments, each sugar in a 3′-end portion independently contains no 2′-OMe.

In some embodiments, a 3′-end portion comprises one or more 2′-F modified sugars.

In some embodiments, a high level (e.g., about 60-100%, or about 60%, 65%, 70%, 75%, 80%, 85%, 90%, 95% or more, or 100%) or all sugars in a 3′-end are independently 2′-F modified sugars, sugars comprising two 2′-H (e.g., natural DNA sugars), or sugars comprising 2′-OH (e.g., natural RNA sugars). In some embodiments, a high level (e.g., about 60-100%, or about 60%, 65%, 70%, 75%, 80%, 85%, 90%, 95% or more, or 100%) or all sugars in a 3′-end portion are independently 2′-F modified sugars, natural DNA sugars, or natural RNA sugars. In some embodiments, a high level (e.g., about 60-100%, or about 60%, 65%, 70%, 75%, 80%, 85%, 90%, 95% or more, or 100%) or all sugars in a 3′-end portion are independently 2′-F modified sugars and natural DNA sugars. In some embodiments, a level is 100%. In some embodiments, sugars of a 3′-end portion are selected from sugars having two 2′-H (e.g., natural DNA sugar) and 2′-F modified sugars. In some embodiments, a 3′-end portion comprise 1, 2, 3, 4 or 5 2′—F modified sugars. In some embodiments, a 3′-end portion comprise 1, 2, 3, 4 or 5 sugars comprising two 2′-H. In some embodiments, a 3′-end portion comprise 1, 2, 3, 4 or 5 natural DNA sugars. In some embodiments, a 3′-end portion comprise 1, 2, 3, 4 or 5 sugars comprising 2′-OH. In some embodiments, a 3′-end portion comprise 1, 2, 3, 4 or 5 natural RNA sugars. In some embodiments, a number is 1. In some embodiments, a number is 2. In some embodiments, a number is 3. In some embodiments, a number is 4. In some embodiments, a number is 5.

In some embodiments, one or more (e.g., about 1, 2, 3, 4, or 5) internucleotidic linkages of a 3′-end portion are independently a modified internucleotidic linkage. In some embodiments, one or more (e.g., about 1, 2, 3, 4, or 5) internucleotidic linkages of a 3′-end portion are independently a chiral internucleotidic linkage. In some embodiments, one or more (e.g., about 1, 2, 3, 4, or 5) internucleotidic linkages of a 3′-end portion are independently a chirally controlled internucleotidic linkage. In some embodiments, one or more (e.g., about 1, 2, 3, 4, or 5) internucleotidic linkages of a 3′-end portion are Rp. In some embodiments, one or more (e.g., about 1, 2, 3, 4, or 5) internucleotidic linkages of a 3′-end portion are Sp. In some embodiments, each internucleotidic linkage of a 3′-end portion is Sp.

In some embodiments, one or more (e.g., about 1, 2, 3, 4, or 5) internucleotidic linkages of a 3′-end portion are independently a modified internucleotidic linkage. In some embodiments, one or more (e.g., about 1, 2, 3, 4, or 5) internucleotidic linkages of a 3′-end portion are independently a chiral internucleotidic linkage. In some embodiments, one or more (e.g., about 1, 2, 3, 4, or 5) internucleotidic linkages of a 3′-end portion are independently a chirally controlled internucleotidic linkage. In some embodiments, one or more (e.g., about 1, 2, 3, 4, or 5) internucleotidic linkages of a 3′-end portion are Rp. In some embodiments, one or more (e.g., about 1, 2, 3, 4, or 5) internucleotidic linkages of a 3′-end portion are Rp. In some embodiments, each internucleotidic linkage of a 3′-end portion is Rp.

In some embodiments, a 3′-end portion comprises one or more (e.g., about 1, 2, 3, 4, or 5) mismatches as described herein. In some embodiments, a 3′-end portion comprises one or more (e.g., about 1, 2, 3, 4, or 5) wobbles as described herein. In some embodiments, a 3′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a 3′-end portion comprises a nucleoside 3′ next to an opposite nucleoside. In some embodiments, a nucleoside 3′ next to an opposite nucleoside comprise a nucleobase as described herein. In some embodiments, a nucleoside 3′ next to an opposite nucleoside forms a wobble pair with a corresponding nucleoside in a target nucleic acid. In some embodiments, the nucleobase of a nucleoside 3′ next to an opposite nucleoside is hypoxanthine; in some embodiments, it is a derivative of hypoxanthine.

In some embodiments, a second subdomain recruits, promotes or contribute to recruitment of, a protein such as an ADAR protein, e.g., ADAR1, ADAR2, etc. In some embodiments, a second subdomain recruits, or promotes or contribute to interactions with, a protein such as an ADAR protein. In some embodiments, a second subdomain contacts with a RNA binding domain (RBD) of ADAR. In some embodiments, a second subdomain contacts with a catalytic domain of ADAR which has a deaminase activity. In some embodiments, a second subdomain contact with a domain that has a deaminase activity of ADAR1. In some embodiments, a second subdomain contact with a domain that has a deaminase activity of ADAR2. In some embodiments, various nucleobases, sugars and/or internucleotidic linkages of a second subdomain may interact with one or more residues of proteins, e.g., ADAR proteins.

Third Subdomains

As described herein, in some embodiment, an oligonucleotide comprises a first domain and a second domain from 5′ to 3′. In some embodiments, a second domain comprises or consists of a first subdomain, a second subdomain, and a third subdomain from 5′ to 3′. Certain embodiments of a third subdomain are described below as examples.

In some embodiments, a third subdomain has a length of about 1-50, 1-40, 1-30, 1-20 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 1, 2, 3, 4, 5, 6, 7, 8, 9, 10, 11, 12, 13, 14, 15, 16, 17, 18, 19, 20, 21, 22, 23, 24, 25, 30, 40 or 50, etc.) nucleobases. In some embodiments, a third subdomain has a length of about 5-30 nucleobases. In some embodiments, a third subdomain has a length of about 10-30 nucleobases. In some embodiments, a third subdomain has a length of about 10-20 nucleobases. In some embodiments, a third subdomain has a length of about 5-15 nucleobases. In some embodiments, a third subdomain has a length of about 13-16 nucleobases. In some embodiments, a third subdomain has a length of about 6-12 nucleobases. In some embodiments, a third subdomain has a length of about 6-9 nucleobases. In some embodiments, a third subdomain has a length of about 1-10 nucleobases. In some embodiments, a third subdomain has a length of about 1-7 nucleobases. In some embodiments, a third subdomain has a length of 1 nucleobase. In some embodiments, a third subdomain has a length of 2 nucleobases. In some embodiments, a third subdomain has a length of 3 nucleobases. In some embodiments, a third subdomain has a length of 4 nucleobases. In some embodiments, a third subdomain has a length of 5 nucleobases. In some embodiments, a third subdomain has a length of 6 nucleobases. In some embodiments, a third subdomain has a length of 7 nucleobases. In some embodiments, a third subdomain has a length of 8 nucleobases. In some embodiments, a third subdomain has a length of 9 nucleobases. In some embodiments, a third subdomain has a length of 10 nucleobases. In some embodiments, a third subdomain has a length of 11 nucleobases. In some embodiments, a third subdomain has a length of 12 nucleobases. In some embodiments, a third subdomain has a length of 13 nucleobases. In some embodiments, a third subdomain has a length of 14 nucleobases. In some embodiments, a third subdomain has a length of 15 nucleobases. In some embodiments, a third subdomain is shorter than a first subdomain. In some embodiments, a third subdomain is shorter than a first domain. In some embodiments, a third subdomain comprises a 3′-end nucleobase of a second domain.

In some embodiments, a third subdomain is about, or at least about, 5-95%, 10%-90%, 20%-80%, 30%-70%, 40%-70%, 40%-60%, 5%, 10%, 15%, 20%, 25%, 30%, 35%, 40%, 45%, 50%, 55%, 60%, 65%, 70%, 75%, 80%, 85%, 90%, 95% of a second domain. In some embodiments, a percentage is about 30%-80%. In some embodiments, a percentage is about 30%-70%. In some embodiments, a percentage is about 40%-60%. In some embodiments, a percentage is about 20%. In some embodiments, a percentage is about 25%. In some embodiments, a percentage is about 30%. In some embodiments, a percentage is about 35%. In some embodiments, a percentage is about 40%. In some embodiments, a percentage is about 45%. In some embodiments, a percentage is about 50%. In some embodiments, a percentage is about 55%. In some embodiments, a percentage is about 60%. In some embodiments, a percentage is about 65%. In some embodiments, a percentage is about 70%. In some embodiments, a percentage is about 75%. In some embodiments, a percentage is about 80%. In some embodiments, a percentage is about 85%. In some embodiments, a percentage is about 90%.

In some embodiments, one or more (e.g., 1-20, 1, 2, 3, 4, 5, 6, 7, 8, 9, or 10, etc.) mismatches exist in a third subdomain when an oligonucleotide is aligned with a target nucleic acid for complementarity. In some embodiments, there is 1 mismatch. In some embodiments, there are 2 mismatches. In some embodiments, there are 3 mismatches. In some embodiments, there are 4 mismatches. In some embodiments, there are 5 mismatches. In some embodiments, there are 6 mismatches. In some embodiments, there are 7 mismatches. In some embodiments, there are 8 mismatches. In some embodiments, there are 9 mismatches. In some embodiments, there are 10 mismatches.

In some embodiments, one or more (e.g., 1-20, 1, 2, 3, 4, 5, 6, 7, 8, 9, or 10, etc.) wobbles exist in a third subdomain when an oligonucleotide is aligned with a target nucleic acid for complementarity. In some embodiments, there is 1 wobble. In some embodiments, there are 2 wobbles. In some embodiments, there are 3 wobbles. In some embodiments, there are 4 wobbles. In some embodiments, there are 5 wobbles. In some embodiments, there are 6 wobbles. In some embodiments, there are 7 wobbles. In some embodiments, there are 8 wobbles. In some embodiments, there are 9 wobbles. In some embodiments, there are 10 wobbles.

In some embodiments, duplexes of oligonucleotides and target nucleic acids in a third subdomain region comprise one or more bulges each of which independently comprise one or more mismatches that are not wobbles. In some embodiments, there are 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) bulges. In some embodiments, the number is 0. In some embodiments, the number is 1. In some embodiments, the number is 2. In some embodiments, the number is 3. In some embodiments, the number is 4. In some embodiments, the number is 5.

In some embodiments, a third subdomain is fully complementary to a target nucleic acid.

In some embodiments, a third subdomain comprises one or more modified nucleobases.

In some embodiments, a third domain comprises a nucleoside opposite to a target adenosine (an opposite nucleoside). In some embodiments, a third domain comprises a nucleoside 3′ next to an opposite nucleoside. In some embodiments, a third domain comprises a nucleoside 5′ next to an opposite nucleoside. Various suitable opposite nucleosides, including sugars and nucleobases thereof, have been described herein.

In some embodiments, a third subdomain comprise a nucleoside opposite to a target adenosine, e.g., when the oligonucleotide forms a duplex with a target nucleic acid. Suitable nucleobases including modified nucleobases in opposite nucleosides are described herein. For example, in some embodiment, an opposite nucleobase is optionally substituted or protected nucleobase selected from C, a tautomer of C, U, a tautomer of U, A, a tautomer of A, and a nucleobase which is or comprises Ring BA having the structure of BA-I, BA-I-a, BA-I-b, BA-II, BA-II-a, BA-II-b, BA-III, BA-III-a, BA-III-b, BA-IV, BA-IV-a, BA-IV-b, BA-V, BA-V-a, BA-V-b, or BA-VI, or a tautomer of Ring BA.

In some embodiments, a third subdomain comprises one or more sugars comprising two 2′-H (e.g., natural DNA sugars). In some embodiments, a third subdomain comprises one or more sugars comprising 2′-OH (e.g., natural RNA sugars). In some embodiments, a third subdomain comprises one or more modified sugars. In some embodiments, a modified sugar comprises a 2′-modification. In some embodiments, a modified sugar is a bicyclic sugar, e.g., a LNA sugar. In some embodiments, a modified sugar is an acyclic sugar (e.g., by breaking a C2-C3 bond of a corresponding cyclic sugar).

In some embodiments, a third subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars. In some embodiments, a third subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently bicyclic sugars (e.g., a LNA sugar) or a 2′-OR modified sugars, wherein R is independently optionally substituted C₁₋₆ aliphatic. In some embodiments, a third subdomain comprises about 1-50, 1-40, 1-30, 1-25, 1-20, 1-15, 1-10 (e.g., about 1, 2, 3, 4, 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars which are independently 2′-OR modified sugars, wherein R is independently optionally substituted C₁₋₆ aliphatic. In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15. In some embodiments, the number is 16. In some embodiments, the number is 17. In some embodiments, the number is 18. In some embodiments, the number is 19. In some embodiments, the number is 20. In some embodiments, R is methyl.

In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising 2′-OH. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising two 2′-H. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) RNA sugars. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) DNA sugars.

In some embodiments, about 5%-100%, (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a third subdomain are independently a modified sugar. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a third subdomain are independently a bicyclic sugar (e.g., a LNA sugar) or a 2′-OR modified sugar, wherein R is independently optionally substituted C₁₋₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all sugars in a third subdomain are independently a 2′-OR modified sugar, wherein R is independently optionally substituted C₁₋₆ aliphatic. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, R is methyl.

In some embodiments, a third subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a third subdomain are independently modified sugars with a modification that is not 2′-F. In some embodiments, about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in a third subdomain are independently modified sugars with a modification that is not 2′-F. In some embodiments, modified sugars of a third subdomain are each independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, a third subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a third subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic. In some embodiments, about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in a third subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, each sugar in a third subdomain independently comprises a 2′-OR modification, wherein R is optionally substituted C₁₋₆ aliphatic, or a 2′-O-L^{B}-4′ modification. In some embodiments, each sugar in a third subdomain independently comprises a 2′-OR modification, wherein R is optionally substituted C₁₋₆ aliphatic, or a 2′-O-L^{B}-4′ modification, wherein L^{B} is optionally substituted —CH₂—. In some embodiments, each sugar in a third subdomain independently comprises 2′-OMe.

In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) 2′-F modified sugars. In some embodiments, a third subdomain comprises no 2′-F modified sugars. In some embodiments, a third subdomain comprises one or more bicyclic sugars and/or 2′-OR modified sugars wherein R is not —H. In some embodiments, levels of bicyclic sugars and/or 2′-OR modified sugars wherein R is not —H, individually or combined, are relatively high compared to level of 2′-F modified sugars. In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a third subdomain comprises 2′-F. In some embodiments, no more than about 50% of sugars in a third subdomain comprises 2′-F. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) bicyclic sugars, e.g., LNA sugars. In some embodiments, a third subdomain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).

In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in a third subdomain comprises 2′-MOE. In some embodiments, no more than about 50% of sugars in a third subdomain comprises 2′-MOE. In some embodiments, no sugars in a third subdomain comprises 2′-MOE. In some embodiments, a third subdomain comprise about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in a third subdomain are modified internucleotidic linkages. In some embodiments, each internucleotidic linkage in a third subdomain is independently a modified internucleotidic linkage. In some embodiments, each modified internucleotidic linkages is independently a chiral internucleotidic linkage. In some embodiments, a modified or chiral internucleotidic linkage is a phosphorothioate internucleotidic linkage. In some embodiments, a modified or chiral internucleotidic linkage is a non-negatively charged internucleotidic linkage. In some embodiments, a modified or chiral internucleotidic linkage is a neutral internucleotidic linkage, e.g., n001. In some embodiments, each modified internucleotidic linkages is independently a phosphorothioate internucleotidic linkage or a non-negatively charged internucleotidic linkage. In some embodiments, each modified internucleotidic linkages is independently a phosphorothioate internucleotidic linkage or a neutral internucleotidic linkage. In some embodiments, each modified internucleotidic linkages is independently a phosphorothioate internucleotidic linkage. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a third subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a third subdomain is chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a third subdomain is chirally controlled. In some embodiments, each is independently chirally controlled. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in a third subdomain is Sp. In some embodiments, each is independently chirally controlled. In some embodiments, at least about 1-50, 1-40, 1-30, 1-25, 1-20, 1-15, 1-10 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) phosphorothioate internucleotidic linkages in a third subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in a third subdomain is Sp. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of phosphorothioate internucleotidic linkages in a third subdomain is Sp. In some embodiments, the number is one or more. In some embodiments, the number is 2 or more. In some embodiments, the number is 3 or more. In some embodiments, the number is 4 or more. In some embodiments, the number is 5 or more. In some embodiments, the number is 6 or more. In some embodiments, the number is 7 or more. In some embodiments, the number is 8 or more. In some embodiments, the number is 9 or more. In some embodiments, the number is 10 or more. In some embodiments, the number is 11 or more. In some embodiments, the number is 12 or more. In some embodiments, the number is 13 or more. In some embodiments, the number is 14 or more. In some embodiments, the number is 15 or more. In some embodiments, a percentage is at least about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, each internucleotidic linkage linking two third subdomain nucleosides is independently a modified internucleotidic linkage. In some embodiments, each modified internucleotidic linkages is independently a chiral internucleotidic linkage. In some embodiments, each modified internucleotidic linkages is independently a phosphorothioate internucleotidic linkage. In some embodiments, each chiral internucleotidic linkage is independently a phosphorothioate internucleotidic linkage. In some embodiments, each modified internucleotidic linkages is independently a Sp chiral internucleotidic linkage. In some embodiments, each modified internucleotidic linkages is independently a Sp phosphorothioate internucleotidic linkage. In some embodiments, each chiral internucleotidic linkages is independently a Sp phosphorothioate internucleotidic linkage. In some embodiments, an internucleotidic linkage of a third subdomain is bonded to two nucleosides of the third subdomain. In some embodiments, an internucleotidic linkage bonded to a nucleoside in a third subdomain and a nucleoside in a second subdomain may be properly considered an internucleotidic linkage of a third subdomain. In some embodiments, an internucleotidic linkage bonded to a nucleoside in a third subdomain and a nucleoside in a second subdomain is a modified internucleotidic linkage; in some embodiments, it is a chiral internucleotidic linkage; in some embodiments, it is chirally controlled; in some embodiments, it is Rp; in some embodiments, it is Sp.

In some embodiments, a third subdomain comprises a certain level of Rp internucleotidic linkages. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all internucleotidic linkages in a third subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chiral internucleotidic linkages in a third subdomain. In some embodiments, a level is about e.g., about 5%-100%, about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc. of all chirally controlled internucleotidic linkages in a third subdomain. In some embodiments, a percentage is about or no more than about 50%. In some embodiments, a percentage is at least about 55%. In some embodiments, a percentage is at least about 60%. In some embodiments, a percentage is at least about 65%. In some embodiments, a percentage is at least about 70%. In some embodiments, a percentage is at least about 75%. In some embodiments, a percentage is at least about 80%. In some embodiments, a percentage is at least about 85%. In some embodiments, a percentage is at least about 90%. In some embodiments, a percentage is at least about 95%. In some embodiments, a percentage is about 100%. In some embodiments, a percentage is about or no more than about 5%. In some embodiments, a percentage is about or no more than about 10%. In some embodiments, a percentage is about or no more than about 15%. In some embodiments, a percentage is about or no more than about 20%. In some embodiments, a percentage is about or no more than about 25%. In some embodiments, a percentage is about or no more than about 30%. In some embodiments, a percentage is about or no more than about 35%. In some embodiments, a percentage is about or no more than about 40%. In some embodiments, a percentage is about or no more than about 45%. In some embodiments, a percentage is about or no more than about 50%. In some embodiments, about 1-50, 1-40, 1-30, 1-25, 1-20, 1-15, 1-10, 1-5, e.g., about 1, 2, 3, 4, 5, 6, 7, 8, 9, 10, 11, 12, 13, 14, 15, 16, 17, 18, 19, or 20 internucleotidic linkages are independently Rp chiral internucleotidic linkages. In some embodiments, the number is about or no more than about 1. In some embodiments, the number is about or no more than about 2. In some embodiments, the number is about or no more than about 3. In some embodiments, the number is about or no more than about 4. In some embodiments, the number is about or no more than about 5. In some embodiments, the number is about or no more than about 6. In some embodiments, the number is about or no more than about 7. In some embodiments, the number is about or no more than about 8. In some embodiments, the number is about or no more than about 9. In some embodiments, the number is about or no more than about 10.

In some embodiments, each phosphorothioate internucleotidic linkage in a third subdomain is independently chirally controlled. In some embodiments, each is independently Sp or Rp. In some embodiments, a high level is Sp as described herein. In some embodiments, each phosphorothioate internucleotidic linkage in a third subdomain is chirally controlled and is Sp. In some embodiments, one or more, e.g., about 1-5 (e.g., about 1, 2, 3, 4, or 5) is Rp.

In some embodiments, as illustrated in certain examples, a third subdomain comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in a third subdomain is about 1-10, or about 1, 2, 3, 4, 5, 6, 7, 8, 9, or 10. In some embodiments, it is about 1. In some embodiments, it is about 2. In some embodiments, it is about 3. In some embodiments, it is about 4. In some embodiments, it is about 5. In some embodiments, two or more non-negatively charged internucleotidic linkages are consecutive. In some embodiments, no two non-negatively charged internucleotidic linkages are consecutive. In some embodiments, all non-negatively charged internucleotidic linkages in a third subdomain are consecutive (e.g., 3 consecutive non-negatively charged internucleotidic linkages). In some embodiments, a non-negatively charged internucleotidic linkage, or two or more (e.g., about 2, about 3, about 4 etc.) consecutive non-negatively charged internucleotidic linkages, are at the 3′-end of a third subdomain. In some embodiments, the last two or three or four internucleotidic linkages of a third subdomain comprise at least one internucleotidic linkage that is not a non-negatively charged internucleotidic linkage. In some embodiments, the last two or three or four internucleotidic linkages of a third subdomain comprise at least one internucleotidic linkage that is not n001. In some embodiments, the internucleotidic linkage linking the last two nucleosides of a third subdomain is a non-negatively charged internucleotidic linkage. In some embodiments, the internucleotidic linkage linking the last two nucleosides of a third subdomain is a Sp non-negatively charged internucleotidic linkage. In some embodiments, the internucleotidic linkage linking the last two nucleosides of a third subdomain is a Rp non-negatively charged internucleotidic linkage. In some embodiments, the internucleotidic linkage linking the last two nucleosides of a third subdomain is a phosphorothioate internucleotidic linkage. In some embodiments, the internucleotidic linkage linking the last two nucleosides of a third subdomain is a Sp phosphorothioate internucleotidic linkage. In some embodiments, the last two nucleosides of a third subdomain are the last two nucleosides of a second domain. In some embodiments, the last two nucleosides of a third subdomain are the last two nucleosides of an oligonucleotide. In some embodiments, the internucleotidic linkage linking the first two nucleosides of a third subdomain is a non-negatively charged internucleotidic linkage. In some embodiments, the internucleotidic linkage linking the first two nucleosides of a third subdomain is a Sp non-negatively charged internucleotidic linkage. In some embodiments, the internucleotidic linkage linking the first two nucleosides of a third subdomain is a Rp non-negatively charged internucleotidic linkage. In some embodiments, the internucleotidic linkage linking the first two nucleosides of a third subdomain is a phosphorothioate internucleotidic linkage. In some embodiments, the internucleotidic linkage linking the first two nucleosides of a third subdomain is a Sp phosphorothioate internucleotidic linkage. In some embodiments, a non-negatively charged internucleotidic linkage is a neutral internucleotidic linkage such as n001.

In some embodiments, a third subdomain comprises one or more (e.g., about 1, 2, 3, 4, 5, 6, 7, 8, 9, 10) natural phosphate linkages. In some embodiments, a third domain contains no natural phosphate linkages.

In some embodiments, a third subdomain comprises a 5′-end portion, e.g., one having a length of about 1-20, 1-15, 1-10, 1-8, 1-5, 1-3, 3-8, or about 1, 2, 3, 4, 5, 6, 7, 8, 9, or 10 nucleobases. In some embodiments, a 5′-end portion has a length of about 1-3 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases. In some embodiments, a length is 6 nucleobases. In some embodiments, a length is 7 nucleobases. In some embodiments, a length is 8 nucleobases. In some embodiments, a length is 9 nucleobases. In some embodiments, a length is 10 nucleobases. In some embodiments, a 5′-end portion comprises the 5′-end nucleobase of a third subdomain. In some embodiments, a third subdomain comprises or consists of a 3′-end portion and a 5′-end portion. In some embodiments, a 5′-end portion comprises the 5′-end nucleobase of a third subdomain. In some embodiments, a 5′-end portion of a third subdomain is bonded to a second subdomain.

In some embodiments, a 5′-end portion comprises one or more sugars having two 2′-H (e.g., natural DNA sugars). In some embodiments, a 5′-end portion comprises one or more sugars having 2′-OH (e.g., natural RNA sugars). In some embodiments, one or more (e.g., about 1-20, 1-15, 1-10, 3-8, or about 1, 2, 3, 4, 5, 6, 7, 8, 9, or 10) of sugars in a 5′-end portion are independently modified sugars. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 5′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, one or more of the modified sugars independently comprises 2′-F or 2′-OR, wherein R is independently optionally substituted C₁₋₆ aliphatic. In some embodiments, one or more of the modified sugars are independently 2′-F or 2′-OMe. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a bicyclic sugar (e.g., a LNA sugar) or a sugar with a 2′-OR modification wherein R is optionally substituted C₁₋₆ aliphatic. In some embodiments, each modified sugar in a 5′-end portion is independently a sugar with a 2′-OR modification wherein R is optionally substituted C₁₋₆ aliphatic. In some embodiments, R is methyl.

In some embodiments, compared to a 3′-end portion, 5′ end portion contains a higher level (in numbers and/or percentage) of 2′-F modified sugars and/or sugars comprising two 2′-H (e.g., natural DNA sugars), and/or a lower level (in numbers and/or percentage) of other types of modified sugars, e.g., bicyclic sugars and/or sugars with 2′-OR modifications wherein R is independently optionally substituted C₁₋₆ aliphatic. In some embodiments, compared to a 3′-end portion, a 5′-end portion contains a higher level of 2′-F modified sugars and/or a lower level of 2′-OR modified sugars wherein R is optionally substituted C₁₋₆ aliphatic. In some embodiments, compared to a 3′-end portion, a 5′-end portion contains a higher level of 2′-F modified sugars and/or a lower level of 2′-OMe modified sugars. In some embodiments, compared to a 3′-end portion, a 5′-end portion contains a higher level of natural DNA sugars and/or a lower level of 2′-OR modified sugars wherein R is optionally substituted C₁₋₆ aliphatic. In some embodiments, compared to a 3′-end portion, a 5′-end portion contains a higher level of natural DNA sugars and/or a lower level of 2′-OMe modified sugars. In some embodiments, a 5′-end portion contains low levels (e.g., no more than 50%, 40%, 30%, 25%, 20%, or 10%, or no more than 1, 2, 3, 4, 5, 6, 7, 8, 9, or 10) of modified sugars which are bicyclic sugars or sugars comprising 2′-OR wherein R is optionally substituted C₁₋₆ aliphatic (e.g., methyl). In some embodiments, a 5′-end portion contains no modified sugars which are bicyclic sugars or sugars comprising 2′-OR wherein R is optionally substituted C₁₋₆ aliphatic (e.g., methyl).

In some embodiments, one or more modified sugars independently comprise 2′-F. In some embodiments, no modified sugars comprises 2′-OMe or other 2′-OR modifications wherein R is optionally substituted C₁₋₆ aliphatic. In some embodiments, each sugar of a 5′-end portion independently comprises two 2′-H or a 2′-F modification. In some embodiments, a 5′-end portion comprises 1, 2, 3, 4, or 5 2′—F modified sugars. In some embodiments, a 5′-end portion comprises 1-3 2′—F modified sugars. In some embodiments, a 5′-end portion comprises 1, 2, 3, 4, or 5 natural DNA sugars. In some embodiments, a 5′-end portion comprises 1-3 natural DNA sugars.

In some embodiments, one or more (e.g., about 1-10, or about 1, 2, 3, 4, 5, 6, 7, 8, 9, or 10) internucleotidic linkages of a 5′-end portion are independently a modified internucleotidic linkage. In some embodiments, one or more (e.g., about 1-10, or about 1, 2, 3, 4, 5, 6, 7, 8, 9, or 10) internucleotidic linkages of a 5′-end portion are independently a chiral internucleotidic linkage. In some embodiments, one or more (e.g., about 1-10, or about 1, 2, 3, 4, 5, 6, 7, 8, 9, or 10) internucleotidic linkages of a 5′-end portion are independently a chirally controlled internucleotidic linkage. In some embodiments, one or more (e.g., about 1-10, or about 1, 2, 3, 4, 5, 6, 7, 8, 9, or 10) internucleotidic linkages of a 5′-end portion are Rp. In some embodiments, one or more (e.g., about 1-10, or about 1, 2, 3, 4, 5, 6, 7, 8, 9, or 10) internucleotidic linkages of a 5′-end portion are Sp. In some embodiments, each internucleotidic linkage of a 5′-end portion is Sp. In some embodiments, a 5′-end portion contains a higher level (in number and/or percentage) of Rp internucleotidic linkage and/or natural phosphate linkage compared to a 3′-end portion.

In some embodiments, a 5′-end portion comprises one or more (e.g., about 1-10, or about 1, 2, 3, 4, 5, 6, 7, 8, 9, or 10) mismatches as described herein. In some embodiments, a 5′-end portion comprises one or more (e.g., about 1-10, or about 1, 2, 3, 4, 5, 6, 7, 8, 9, or 10) wobbles as described herein. In some embodiments, a 5′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a third subdomain comprises a 3′-end portion, e.g., one having a length of about 1-20, 1-15, 1-10, 1-8, 1-4, 3-8, or about 1, 2, 3, 4, 5, 6, 7, 8, 9, or 10 nucleobases. In some embodiments, a 3′-end portion has a length of about 3-6 nucleobases. In some embodiments, a length is one nucleobase. In some embodiments, a length is 2 nucleobases. In some embodiments, a length is 3 nucleobases. In some embodiments, a length is 4 nucleobases. In some embodiments, a length is 5 nucleobases. In some embodiments, a length is 6 nucleobases. In some embodiments, a length is 7 nucleobases. In some embodiments, a length is 8 nucleobases. In some embodiments, a length is 9 nucleobases. In some embodiments, a length is 10 nucleobases. In some embodiments, a 3′-end portion comprises the 3′-end nucleobase of a third subdomain.

In some embodiments, a 3′-end portion comprises one or more sugars having two 2′-H (e.g., natural DNA sugars). In some embodiments, a 3′-end portion comprises one or more sugars having 2′-OH (e.g., natural RNA sugars). In some embodiments, one or more (e.g., about 1-20, 1-15, 1-10, 3-8, or about 1, 2, 3, 4, 5, 6, 7, 8, 9, or 10) of sugars in a 3′-end portion are independently modified sugars. In some embodiments, about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in a 3′-end portion are independently modified sugars. In some embodiments, each sugar is independently a modified sugar. In some embodiments, modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.

In some embodiments, one or more of the modified sugars independently comprises 2′-F or 2′-OR, wherein R is independently optionally substituted C₁₋₆ aliphatic. In some embodiments, one or more of the modified sugars are independently 2′-F or 2′-OMe. In some embodiments, each modified sugar in a 3′-end portion is independently a bicyclic sugar (e.g., a LNA sugar) or a sugar with a 2′-OR modification wherein R is optionally substituted C₁₋₆ aliphatic. In some embodiments, each modified sugar in a 3′-end portion is independently a bicyclic sugar (e.g., a LNA sugar) or a sugar with a 2′-OR modification wherein R is optionally substituted C₁₋₆ aliphatic. In some embodiments, each modified sugar in a 3′-end portion is independently a sugar with a 2′-OR modification wherein R is optionally substituted C₁₋₆ aliphatic. In some embodiments, R is methyl.

In some embodiments, one or more sugars in a 3′-end portion independently comprise a 2′-OR modification, wherein R is optionally substituted C₁₋₆ aliphatic, or a 2′-O-L^{B}-4′ modification. In some embodiments, each sugar in a 3′-end portion independently comprises a 2′-OR modification, wherein R is optionally substituted C₁₋₆ aliphatic, or a 2′-O-L^{B}-4′ modification. In some embodiments, L^{B} is optionally substituted —CH₂—. In some embodiments, L^{B} is —CH₂—. In some embodiments, each sugar in a 3′-end portion independently comprises 2′-OMe.

In some embodiments, one or more (e.g., about 1-10, or about 1, 2, 3, 4, 5, 6, 7, 8, 9, or 10) internucleotidic linkages of a 3′-end portion are independently a modified internucleotidic linkage. In some embodiments, one or more (e.g., about 1-10, or about 1, 2, 3, 4, 5, 6, 7, 8, 9, or 10) internucleotidic linkages of a 3′-end portion are independently a chiral internucleotidic linkage. In some embodiments, one or more (e.g., about 1-10, or about 1, 2, 3, 4, 5, 6, 7, 8, 9, or 10) internucleotidic linkages of a 3′-end portion are independently a chirally controlled internucleotidic linkage. In some embodiments, one or more (e.g., about 1-10, or about 1, 2, 3, 4, 5, 6, 7, 8, 9, or 10) internucleotidic linkages of a 3′-end portion are Rp. In some embodiments, one or more (e.g., about 1-10, or about 1, 2, 3, 4, 5, 6, 7, 8, 9, or 10) internucleotidic linkages of a 3′-end portion are Sp. In some embodiments, each internucleotidic linkage of a 3′-end portion is Sp.

In some embodiments, a 3′-end portion comprises one or more (e.g., about 1-10, or about 1, 2, 3, 4, 5, 6, 7, 8, 9, or 10) mismatches as described herein. In some embodiments, a 3′-end portion comprises one or more (e.g., about 1-10, or about 1, 2, 3, 4, 5, 6, 7, 8, 9, or 10) wobbles as described herein. In some embodiments, a 3′-end portion is about 60-100% (e.g., 66%, 70%, 75%, 80%, 85%, 90%, 95%, or more) complementary to a target nucleic acid. In some embodiments, a complementarity is 60% or more. In some embodiments, a complementarity is 70% or more. In some embodiments, a complementarity is 75% or more. In some embodiments, a complementarity is 80% or more. In some embodiments, a complementarity is 90% or more.

In some embodiments, a third subdomain recruits, promotes or contribute to recruitment of, a protein such as an ADAR protein, e.g., ADAR1, ADAR2, etc. In some embodiments, a third subdomain recruits, or promotes or contribute to interactions with, a protein such as an ADAR protein. In some embodiments, a third subdomain contacts with a RNA binding domain (RBD) of ADAR. In some embodiments, a third subdomain contacts with a catalytic domain of ADAR which has a deaminase activity. In some embodiments, a third subdomain contact with a domain that has a deaminase activity of ADAR1. In some embodiments, a third subdomain contact with a domain that has a deaminase activity of ADAR2. In some embodiments, various nucleobases, sugars and/or internucleotidic linkages of a third subdomain may interact with one or more residues of proteins, e.g., ADAR proteins.

As demonstrated herein, chiral control of linkage phosphorus of chiral internucleotidic linkages can be utilized in oligonucleotides to provide various properties and/or activities. In some embodiments, a Rp internucleotidic linkage (e.g., a Rp phosphorothioate internucleotidic linkage), a Sp internucleotidic linkage (e.g., a Rp phosphorothioate internucleotidic linkage), or a non-chirally controlled internucleotidic linkage (e.g., a non-chirally controlled phosphorothioate internucleotidic linkage) is at one or more of positions −8 , −7 , −6 , −5 , −4 , −3 , −2 , −1, +1, +2, +3, +4, +5, +6, +7, and +8 of a nucleoside opposite to a target adenosine (“+” is counting from the nucleoside toward the 5′-end of an oligonucleotide with the internucleotidic linkage at the +1 position being the internucleotidic linkage bonded to the 5′-carbon of the nucleoside, and “−” is counting from the nucleoside toward the 3′-end of an oligonucleotide with the internucleotidic linkage at the −1 position being the internucleotidic linkage bonded to the 3′-carbon). In some embodiments, a Rp internucleotidic linkage (e.g., a Rp phosphorothioate internucleotidic linkage) is at one or more of positions −8 , −7 , −6 , −5 , −4 , −3 , −2 , −1, +1, +2, +3, +4, +5, +6, +7, and +8 of a nucleoside opposite to a target adenosine. In some embodiments, a Rp internucleotidic linkage (e.g., a Rp phosphorothioate internucleotidic linkage) is at one or more of positions −2 , −1, +3, +4, +5, +6, +7, and +8 of a nucleoside opposite to a target adenosine. In some embodiments, a Sp internucleotidic linkage (e.g., a Sp phosphorothioate internucleotidic linkage) is at one or more of positions −8, −7, −6, −5, −4, −3, −2, −1, +1, +2, +3, +4, +5, +6, +7, and +8 of a nucleoside opposite to a target adenosine. In some embodiments, a Sp internucleotidic linkage (e.g., a Sp phosphorothioate internucleotidic linkage) is at one or more of positions −2, −1, +3, +4, +5, +6, +7, and +8 of a nucleoside opposite to a target adenosine. In some embodiments, a non-chirally controlled internucleotidic linkage (e.g., a non-chirally controlled phosphorothioate internucleotidic linkage) is at one or more of positions −8, −7, −6, −5, −4, −3, −2, −1, +1, +2, +3, +4, +5, +6, +7, and +8 of a nucleoside opposite to a target adenosine. In some embodiments, a non-chirally controlled internucleotidic linkage (e.g., a non-chirally controlled phosphorothioate internucleotidic linkage) is at one or more of positions −2 , −1, +3, +4, +5, +6, +7, and +8 of a nucleoside opposite to a target adenosine.

In some embodiments, Rp is at position +8. In some embodiments, Rp is at position +7. In some embodiments, Rp is at position −6. In some embodiments, Rp is at position +5. In some embodiments, Rp is at position +4. In some embodiments, Rp is at position +3. In some embodiments, Rp is at position +2. In some embodiments, Rp is at position +1. In some embodiments, Rp is at position −1. In some embodiments, Rp is at position −2. In some embodiments, Rp is at position −3. In some embodiments, Rp is at position −4. In some embodiments, Rp is at position −5. In some embodiments, Rp is at position −6. In some embodiments, Rp is at position −7. In some embodiments, Rp is at position −8. In some embodiments, Rp is the configuration of a chirally controlled phosphorothioate internucleotidic linkage. In some embodiments, Sp is at position +8. In some embodiments, Sp is at position +7. In some embodiments, Sp is at position −6. In some embodiments, Sp is at position +5. In some embodiments, Sp is at position +4. In some embodiments, Sp is at position +3. In some embodiments, Sp is at position +2. In some embodiments, Sp is at position +1. In some embodiments, Sp is at position −1. In some embodiments, Sp is at position −2. In some embodiments, Sp is at position −3. In some embodiments, Sp is at position −4. In some embodiments, Sp is at position −5. In some embodiments, Sp is at position −6. In some embodiments, Sp is at position −7. In some embodiments, Sp is at position −8. In some embodiments, Sp is the configuration of a chirally controlled phosphorothioate internucleotidic linkage. In some embodiments, a non-chirally controlled internucleotidic linkage is at position +8. In some embodiments, a non-chirally controlled internucleotidic linkage is at position +7. In some embodiments, a non-chirally controlled internucleotidic linkage is at position −6. In some embodiments, a non-chirally controlled internucleotidic linkage is at position +5. In some embodiments, a non-chirally controlled internucleotidic linkage is at position +4. In some embodiments, a non-chirally controlled internucleotidic linkage is at position +3. In some embodiments, a non-chirally controlled internucleotidic linkage is at position +2. In some embodiments, a non-chirally controlled internucleotidic linkage is at position +1. In some embodiments, a non-chirally controlled internucleotidic linkage is at position −1. In some embodiments, a non-chirally controlled internucleotidic linkage is at position −2. In some embodiments, a non-chirally controlled internucleotidic linkage is at position −3. In some embodiments, a non-chirally controlled internucleotidic linkage is at position −4. In some embodiments, a non-chirally controlled internucleotidic linkage is at position −5. In some embodiments, a non-chirally controlled internucleotidic linkage is at position −6. In some embodiments, a non-chirally controlled internucleotidic linkage is at position −7. In some embodiments, a non-chirally controlled internucleotidic linkage is at position −8. In some embodiments, a non-chirally controlled internucleotidic linkage is a non-chirally controlled phosphorothioate internucleotidic linkage.

In some embodiments, a first domain comprises one or more (e.g., 1, 2, 3, 4, 5, 6, 7, 8, 9, or 10) Rp internucleotidic linkages (e.g., Rp phosphorothioate internucleotidic linkages). In some embodiments, a first domain comprises one or more (e.g., 1, 2, 3, 4, 5, 6, 7, 8, 9, or 10) Sp internucleotidic linkages (e.g., Sp phosphorothioate internucleotidic linkages). In some embodiments, a first domain comprises one or more (e.g., 1, 2, 3, 4, 5, 6, 7, 8, 9, or 10) non-chirally controlled internucleotidic linkages (e.g., non-chirally controlled phosphorothioate internucleotidic linkages). In some embodiments, such internucleotidic linkages are consecutive. In some embodiments, at least about 50%, 55%, 60%, 65%, 70%, 75%, 80%, 85%, 90%, or 95%, or all of internucleotidic linkages in a first domain are chirally controlled and are Sp. In some embodiments, at least about 50%, 55%, 60%, 65%, 70%, 75%, 80%, 85%, 90%, or 95%, or all of phosphorothioate internucleotidic linkages in a first domain are chirally controlled and are Sp. In some embodiments, a second domain comprises one or more (e.g., 1, 2, 3, 4, 5, 6, 7, 8, 9, or 10) Rp internucleotidic linkages (e.g., Rp phosphorothioate internucleotidic linkages). In some embodiments, a second domain comprises one or more (e.g., 1, 2, 3, 4, 5, 6, 7, 8, 9, or 10) Sp internucleotidic linkages (e.g., Sp phosphorothioate internucleotidic linkages). In some embodiments, a second domain comprises one or more (e.g., 1, 2, 3, 4, 5, 6, 7, 8, 9, or 10) non-chirally controlled internucleotidic linkages (e.g., non-chirally controlled phosphorothioate internucleotidic linkages). In some embodiments, such internucleotidic linkages are consecutive. In some embodiments, at least about 50%, 55%, 60%, 65%, 70%, 75%, 80%, 85%, 90%, or 95%, or all of internucleotidic linkages in a second domain are chirally controlled and are Sp. In some embodiments, at least about 50%, 55%, 60%, 65%, 70%, 75%, 80%, 85%, 90%, or 95%, or all of phosphorothioate internucleotidic linkages in a second domain are chirally controlled and are Sp. In some embodiments, a first subdomain comprises one or more (e.g., 1, 2, 3, 4, 5, 6, 7, 8, 9, or 10) Rp internucleotidic linkages (e.g., Rp phosphorothioate internucleotidic linkages). In some embodiments, a first subdomain comprises one or more (e.g., 1, 2, 3, 4, 5, 6, 7, 8, 9, or 10) Sp internucleotidic linkages (e.g., Sp phosphorothioate internucleotidic linkages). In some embodiments, a first subdomain comprises one or more (e.g., 1, 2, 3, 4, 5, 6, 7, 8, 9, or 10) non-chirally controlled internucleotidic linkages (e.g., non-chirally controlled phosphorothioate internucleotidic linkages). In some embodiments, such internucleotidic linkages are consecutive. In some embodiments, such internucleotidic linkages are at 3′-end portion of a first subdomain.

In some embodiments, one or more natural phosphate linkages are utilized in provided oligonucleotides and compositions thereof. In some embodiments, provided oligonucleotides or portions thereof (e.g., first domains, second domains, first subdomains, second subdomains, third subdomains, etc.) comprise one or more (e.g., about, or at least about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, or more) natural phosphate linkages. In some embodiments, provided oligonucleotides or portions thereof (e.g., first domains, second domains, first subdomains, second subdomains, third subdomains, etc.) comprise two or more (e.g., about, or at least about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, or more) consecutive natural phosphate linkages. In some embodiments, provided oligonucleotides or portions thereof (e.g., first domains, second domains, first subdomains, second subdomains, third subdomains, etc.) comprise no more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 natural phosphate linkages. In some embodiments, provided oligonucleotides or portions thereof (e.g., first domains, second domains, first subdomains, second subdomains, third subdomains, etc.) comprise no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 consecutive natural phosphate linkages. In some embodiments, about or at least about 5%, 10%, 15%, 20%, 25%, 30%, 35%, 40%, 45%, 50%, 55%, 60%, 65%, 70%, 75%, 80%, 85%, 90%, or 95%, or all internucleotidic linkages in provided oligonucleotides or portions thereof (e.g., first domains, second domains, first subdomains, second subdomains, third subdomains, etc.) are natural phosphate linkages. In some embodiments, about or at least about 5%, 10%, 15%, 20%, 25%, 30%, 35%, 40%, 45%, 50%, 55%, 60%, 65%, 70%, 75%, 80%, 85%, 90%, or 95%, or all internucleotidic linkages in provided oligonucleotides or portions thereof (e.g., first domains, second domains, first subdomains, second subdomains, third subdomains, etc.) are not natural phosphate linkages. In some embodiments, about or at least about 5%, 10%, 15%, 20%, 25%, 30%, 35%, 40%, 45%, 50%, 55%, 60%, 65%, 70%, 75%, 80%, 85%, 90%, or 95%, or all internucleotidic linkages in provided oligonucleotides or portions thereof (e.g., first domains, second domains, first subdomains, second subdomains, third subdomains, etc.) are not consecutive natural phosphate linkages.

In some embodiments, provided oligonucleotides or portions thereof comprises one or more natural phosphate linkages and one or more modified internucleotidic linkages. In some embodiments, provided oligonucleotides or portions thereof comprises one or more natural phosphate linkages and one or more chirally controlled modified internucleotidic linkages. In some embodiments, provided oligonucleotides or portions thereof (e.g., first domains, second domains, first subdomains, second subdomains, third subdomains, etc.) comprise no more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 natural phosphate linkages each of which independently bonds to two sugars comprising no 2′-OR modification, wherein R is as described herein but not —H. In some embodiments, provided oligonucleotides or portions thereof (e.g., first domains, second domains, first subdomains, second subdomains, third subdomains, etc.) comprise no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 consecutive natural phosphate linkages each of which independently bonds to two sugars comprising no 2′-OR modification, wherein R is as described herein but not —H. In some embodiments, provided oligonucleotides or portions thereof (e.g., first domains, second domains, first subdomains, second subdomains, third subdomains, etc.) comprise no more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 natural phosphate linkages each of which independently bonds to two 2′-F modified sugars. In some embodiments, provided oligonucleotides or portions thereof (e.g., first domains, second domains, first subdomains, second subdomains, third subdomains, etc.) comprise no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 consecutive natural phosphate linkages each of which independently bonds to two 2′-F modified sugars. In some embodiments, in oligonucleotides or portions thereof (e.g., first domains, second domains, first subdomains, second subdomains, third subdomains, etc.) no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, e.g., no more than 2, no more than 3, no more than 4, no more than 5, etc., internucleotidic linkages that bond to two sugars comprising no 2′-OR modification wherein R is as described herein but not —H are natural phosphate linkages. In some embodiments, in oligonucleotides or portions thereof (e.g., first domains, second domains, first subdomains, second subdomains, third subdomains, etc.) no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, e.g., no more than 2, no more than 3, no more than 4, no more than 5, etc., internucleotidic linkages that bond to two 2′-F modified sugars are natural phosphate linkages. In some embodiments, in oligonucleotides or portions thereof (e.g., first domains, second domains, first subdomains, second subdomains, third subdomains, etc.) no more than about 5%, 10%, 15%, 20%, 25%, 30%, 35%, 40%, 45%, 50%, 55%, 60%, 65%, 70%, 75%, 80%, 85%, 90%, or 95%, e.g., no more than 10%, no more than 15%, no more than 20%, no more than 25%, no more than about 30%, no more than about 40%, no more than 50% etc., of internucleotidic linkages that bond to two sugars comprising no 2′-OR modification wherein R is as described herein but not —H are natural phosphate linkages. In some embodiments, in oligonucleotides or portions thereof (e.g., first domains, second domains, first subdomains, second subdomains, third subdomains, etc.) no more than about 5%, 10%, 15%, 20%, 25%, 30%, 35%, 40%, 45%, 50%, 55%, 60%, 65%, 70%, 75%, 80%, 85%, 90%, or 95%, e.g., no more than 10%, no more than 15%, no more than 20%, no more than 25%, no more than about 30%, no more than about 40%, no more than 50% etc., of internucleotidic linkages that bond to two 2′-F modified sugars are natural phosphate linkages. In some embodiments, in oligonucleotides or portions thereof (e.g., first domains, second domains, first subdomains, second subdomains, third subdomains, etc.) no more than about 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, e.g., no more than 2, no more than 3, no more than 4, no more than 5, etc., consecutive internucleotidic linkages that bond to two sugars comprising no 2′-OR modification wherein R is as described herein but not —H are natural phosphate linkages. In some embodiments, in oligonucleotides or portions thereof (e.g., first domains, second domains, first subdomains, second subdomains, third subdomains, etc.) no more than about 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50, e.g., no more than 2, no more than 3, no more than 4, no more than 5, etc., consecutive internucleotidic linkages that bond to two 2′-F modified sugars are natural phosphate linkages.

In some embodiments, a natural phosphate linkage is at one or more of positions −8, −7, −6, −5, −4, −3, −2, −1, +1, +2, +3, +4, +5, +6, +7, and +8 of a nucleoside opposite to a target adenosine. In some embodiments, a natural phosphate linkage is at one or more of positions−1 and +1. In some embodiments, a natural phosphate linkage is at positions−1 and +1. In some embodiments, a natural phosphate linkage is at position −1. In some embodiments, a natural phosphate linkage is at position +1. In some embodiments, a natural phosphate linkage is at position +8. In some embodiments, a natural phosphate linkage is at position +7. In some embodiments, a natural phosphate linkage is at position −6. In some embodiments, a natural phosphate linkage is at position +5. In some embodiments, a natural phosphate linkage is at position +4. In some embodiments, a natural phosphate linkage is at position +3. In some embodiments, a natural phosphate linkage is at position +2. In some embodiments, a natural phosphate linkage is at position −2. In some embodiments, a natural phosphate linkage is at position −3. In some embodiments, a natural phosphate linkage is at position −4. In some embodiments, a natural phosphate linkage is at position −5. In some embodiments, a natural phosphate linkage is at position −6. In some embodiments, a natural phosphate linkage is at position −7. In some embodiments, a natural phosphate linkage is at position −8. In some embodiments, a natural phosphate linkage is at position −1, and a modified internucleotidic linkage is at position +1. In some embodiments, a natural phosphate linkage is at position +1, and a modified internucleotidic linkage is at position −1. In some embodiments, a modified internucleotidic linkage is chirally controlled. In some embodiments, a modified internucleotidic linkage is chirally controlled and is Sp. In some embodiments, a modified internucleotidic linkage is a chirally controlled Sp phosphorothioate internucleotidic linkage. In some embodiments, a modified internucleotidic linkage is chirally controlled and is Rp. In some embodiments, a modified internucleotidic linkage is a chirally controlled Rp phosphorothioate internucleotidic linkage. In some embodiments, a second domain comprises no more than 2 natural phosphate linkages. In some embodiments, a second domain comprises no more than 1 natural phosphate linkages. In some embodiments, a single natural phosphate linkage can be utilized at various positions of an oligonucleotide or a portion thereof (e.g., a first domain, a second domain, a first subdomain, a second subdomain, a third subdomain, etc.).

In some embodiments, particular types of sugars are utilized at particular positions of oligonucleotides or portions thereof. For example, in some embodiments, a first domain comprises a number of 2′-F modified sugars (and optionally a number of 2′-OR modified sugars wherein R is not-H, in some embodiments at lower levels than 2′-F modified sugars), a first subdomain comprises a number of 2′-OR modified sugars wherein R is not-H (e.g., 2′-OMe modified sugars; and optionally a number of 2′-F sugars, in some embodiments at lower levels than 2′-OR modified sugars wherein R is not —H), a second domain comprises one or more natural DNA sugars (no substitution at 2′ position) and/or one or more 2′-F modified sugars, and/or a third subdomain comprises a number of 2′-OR modified sugars wherein R is not-H (e.g., 2′-OMe modified sugars; and optionally a number of 2′-F sugars, in some embodiments at lower levels than 2′-OR modified sugars wherein R is not —H). In some embodiments, particular type of sugars are independently at one or more of positions −8, −7, −6, −5, −4, −3, −2, −1, 0, +1, +2, +3, +4, +5, +6, +7, and +8 of a nucleoside opposite to a target adenosine (“+” is counting from the nucleoside toward the 5′-end of an oligonucleotide, “−” is counting from the nucleoside toward the 3′-end of an oligonucleotide, with position 0 being the position of the nucleoside opposite to a target adenosine, e.g.: 5′- . . . N₊₂N₋₁N₀N₋₁N₋₂ . . . 3′). In some embodiments, particular types of sugars are independently at one or more of positions −5, −4, −3, −2, −1, 0, +1, +2, +3, +4, and +5. In some embodiments, particular types of sugars are independently at one or more of positions −3, −2, −1, 0, +1, +2, and +3. In some embodiments, particular types of sugars are independently at one or more of positions −2, −1, 0, +1, and +2. In some embodiments, particular types of sugars are independently at one or more of positions −1, 0, and +1. In some embodiments, a particular type of sugar is at position +8. In some embodiments, a particular type of sugar is at position +7. In some embodiments, a particular type of sugar is at position +6. In some embodiments, a particular type of sugar is at position +5. In some embodiments, a particular type of sugar is at position +4. In some embodiments, a particular type of sugar is at position +3. In some embodiments, a particular type of sugar is at position +2. In some embodiments, a particular type of sugar is at position +1. In some embodiments, a particular type of sugar is at position 0. In some embodiments, a particular type of sugar is at position −8. In some embodiments, a particular type of sugar is at position −7. In some embodiments, a particular type of sugar is at position −6. In some embodiments, a particular type of sugar is at position −5. In some embodiments, a particular type of sugar is at position −4. In some embodiments, a particular type of sugar is at position −3. In some embodiments, a particular type of sugar is at position −2. In some embodiments, a particular type of sugar is at position −1. In some embodiments, a particular type of sugar is independently a sugar selected from a natural DNA sugar (two 2′-H at 2′-carbon), a 2′-OMe modified sugar, and a 2′-F modified sugar. In some embodiments, a particular type of sugar is independently a sugar selected from a natural DNA sugar (two 2′-H at 2′-carbon) and a 2′-OMe modified sugar. In some embodiments, a particular type of sugar is independently a sugar selected from a natural DNA sugar (two 2′-H at 2′-carbon) and a 2′-F modified sugar, e.g., for sugars at position 0,−1, and/or +1. In some embodiments, a particularly type of sugar is a natural DNA sugar (two 2′-H at 2′-carbon), e.g., at position −1, 0 or +1. In some embodiments, a particular type of sugar is 2′-F modified sugar, e.g., at position −8, −7, −6, −5, −4, −3, −2, −1, 0, +1, +2, +3, +4, +5, +6, +7, and/or +8. In some embodiments, a particular type of sugar is 2′-F modified sugar, e.g., at position −8, −7, −6, −5, −4, −3, −2, +2, +3, +4, +5, +6, +7, and/or +8. In some embodiments, a 2′-F modified sugar is at position −2. In some embodiments, a 2′-F modified sugar is at position −3. In some embodiments, a 2′-F modified sugar is at position −4. In some embodiments, a 2′-F modified sugar is at position +2. In some embodiments, a 2′-F modified sugar is at position +3. In some embodiments, a 2′-F modified sugar is at position +4. In some embodiments, a 2′-F modified sugar is at position +5. In some embodiments, a 2′-F modified sugar is at position +6. In some embodiments, a 2′-F modified sugar is at position +7. In some embodiments, a 2′-F modified sugar is at position +8. In some embodiments, a particular type of sugar is 2′-OMe modified sugar, e.g., at position −8, −7, −6, −5, −4, −3, −2, −1, 0, +1, +2, +3, +4, +5, +6, +7, and/or +8. In some embodiments, a particular type of sugar is 2′-OMe modified sugar, e.g., at position −8, −7, −6, −5, −4, −3, −2, +2, +3, +4, +5, +6, +7, and/or +8. In some embodiments, a 2′-OMe modified sugar is at position −2. In some embodiments, a 2′-OMe modified sugar is at position −3. In some embodiments, a 2′-OMe modified sugar is at position −4. In some embodiments, a 2′-OMe modified sugar is at position +2. In some embodiments, a 2′-OMe modified sugar is at position +3. In some embodiments, a 2′-OMe modified sugar is at position +4. In some embodiments, a 2′-OMe modified sugar is at position +5. In some embodiments, a 2′-OMe modified sugar is at position +6. In some embodiments, a 2′-OMe modified sugar is at position +7. In some embodiments, a 2′-OMe modified sugar is at position +8. In some embodiments, a sugar at position 0 is not a 2′-MOE modified sugar. In some embodiments, a sugar at position 0 is a natural DNA sugar (two 2′-H at 2′-carbon). In some embodiments, a sugar at position 0 is not a 2′-MOE modified sugar. In some embodiments, a sugar at position −1 is not a 2′-MOE modified sugar. In some embodiments, a sugar at position −2 is not a 2′-MOE modified sugar. In some embodiments, a sugar at position −3 is not a 2′-MOE modified sugar. In some embodiments, a first domain comprises one or more 2′-F modified sugars, and optionally 2′-OR modified sugars (in some embodiments at lower levels than 2′-F modified sugars) wherein R is as described herein and is not —H. In some embodiments, a first domain comprises 1, 2, 3, 4, 5, 6, 7, 8, 9, or 10 2′—OR modified sugars (in some embodiments at lower levels that 2′-F modified sugars) wherein R is as described herein and is not —H. In some embodiments, a first domain comprise 1, 2, 3, or 4, or 1 and no more than 1, 2 and no more than 2, 3 and no more than 3, or 4 and no more than 4 2′—OR modified sugars wherein R is C₁₋₆ aliphatic. In some embodiments, the first, second, third and/or fourth sugars of a first domain are independently 2′-OR modified sugars, wherein R is optionally substituted C₁₋₆ aliphatic. In some embodiments, sugars comprising 2′-OR are consecutive. In some embodiments, a first domain comprises 2, 3, 4, 5, 6, 7, 8, 9, or 10 consecutive sugars at its 5′-end, wherein each sugar independently comprises 2′-OR, wherein R is optionally substituted C₁₋₆ aliphatic. In some embodiments, 2′-OR is 2′-OMe. In some embodiments, 2′-OR is 2′-MOE. In some embodiments, a second domain comprises one or more 2′-OR modified sugars (in some embodiments at lower levels) wherein R is as described herein and is not —H, and optionally 2′-F modified sugars (in some embodiments at lower levels). In some embodiments, a first subdomain comprises one or more 2′-OR modified sugars (in some embodiments at lower levels) wherein R is as described herein and is not —H, and optionally 2′-F modified sugars (in some embodiments at lower levels). In some embodiments, a third subdomain comprises one or more 2′-OR modified sugars (in some embodiments at lower levels) wherein R is as described herein and is not —H, and optionally 2′-F modified sugars (in some embodiments, at lower levels; in some embodiments, at higher levels). In some embodiments, a third subdomain comprises about, or at least about 1, 2, 3, 4, 5, 6, 7, 8, 9, or 10 2′—F modified sugars. In some embodiments, a third subdomain comprises about, or at least about 1, 2, 3, 4, 5, 6, 7, 8, 9, or 10 2′—F consecutive modified sugars. In some embodiments, about or at least about, 50%, 55%, 60%, 65%, 70%, 75%, 80%, 85%, 90%, or 95% of sugars in a third subdomain independently comprise 2′-F modification. In some embodiments, the first 2′-F modified sugar in the third subdomain (from 5′ to 3′) is not the first sugar in the third subdomain. In some embodiments, the first 2′-F modified sugar in the third domain is at position −3 relative to the nucleoside opposition to a target adenosine. In some embodiments, each sugar in a third subdomain is independently a modified sugar. In some embodiments, each sugar in a third subdomain is independently a modified sugar, wherein the modification is selected from 2′-F and 2′-OR, wherein R is C₁₋₆ aliphatic. In some embodiments, a modification in selected from 2′-F and 2′-OMe. In some embodiments, each modified sugar in a third subdomain is independently 2′-F modified sugar. In some embodiments, each modified sugar in a third subdomain is independently 2′-OMe modified sugar. In some embodiments, one or more modified sugars in a third subdomain are independently 2′-OMe modified sugar, and one or more modified sugars in a third subdomain are independently 2′-F modified sugar. In some embodiments, each modified sugar in a third subdomain is independently a 2′-F modified sugar except the first sugar of a third subdomain, which in some embodiments is a 2′-OMe modified sugar. In some embodiments, a third subdomain comprises one or more 2′-OR modified sugars (in some embodiments at lower levels) wherein R is as described herein and is not —H, and optionally 2′-F modified sugars (in some embodiments at lower levels). In some embodiments, 2′-OR is 2′-OMe. In some embodiments, 2′-OR is 2′-MOE.

### Base Sequences

As appreciated by those skilled in the art, structural features of the present disclosure, such as nucleobase modification, sugar modifications, internucleotidic linkage modifications, linkage phosphorus stereochemistry, etc., and combinations thereof may be utilized with various suitable base sequences to provide oligonucleotides and compositions with desired properties and/or activities. For example, oligonucleotides for adenosine modification (e.g., conversion to I in the presence of ADAR proteins) typically have sequences that are sufficiently complementary to sequences of target nucleic acids that comprise target adenosines. Nucleosides opposite to target adenosines can be present at various positions of oligonucleotides. In some embodiments, one or more opposite nucleosides are in first domains. In some embodiments, one or more opposite nucleosides are in second domains. In some embodiments, one or more opposite nucleosides are in first subdomains. In some embodiments, one or more opposite nucleosides are in second subdomains. In some embodiments, one or more opposite nucleosides are in third subdomains. Oligonucleotide of the present disclosure may target one or more target adenosines. In some embodiments, one or more opposite nucleosides are each independently in a portion which has the structure features of a second subdomain, and each independently have one or more or all structural features of opposite nucleosides as described herein. In many embodiments, e.g., for targeting G to A mutations, oligonucleotides may selectively target one and only one target adenosine for modification, e.g., by ADAR to convert into I. In some embodiments, an opposite nucleoside is closer to the 3′-end than to the 5′-end of an oligonucleotide.

In some embodiments, an oligonucleotide has a base sequence described herein (e.g., in Tables) or a portion thereof (e.g., a span of 10-50, 10-40, 10-30, 10-20, or 10, 11, 12, 13, 14, 15, 16, 17, 18, 19, 20, or at least 10, at least 15, at least 20, at least 25 contiguous nucleobases) with 0-5 (e.g., 0, 1, 2, 3, 4 or 5) mismatches, wherein each T can be independently substituted with U and vice versa. In some embodiments, an oligonucleotide comprises a base sequence described herein, or a portion thereof, wherein a portion is a span of at least 10 contiguous nucleobases, or a span of at least 15 contiguous nucleobases with 0-5 mismatches. In some embodiments, provided oligonucleotides have a base sequence described herein, or a portion thereof, wherein a portion is a span of at least 10 contiguous nucleobases, or a span of at least 10 contiguous nucleobases with 1-5 mismatches, wherein each T can be independently substituted with U and vice versa.

In some embodiments, base sequences of oligonucleotides comprise or consist of 10-60 (e.g., about or at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60; in some embodiments, at least 15; in some embodiments, at least 16; in some embodiments, at least 17; in some embodiments, at least 18; in some embodiments, at least 19; in some embodiments, at least 20; in some embodiments, at least 21; in some embodiments, at least 22; in some embodiments, at least 23; in some embodiments, at least 24; in some embodiments, at least 25; in some embodiments, at least 26; in some embodiments, at least 27; in some embodiments, at least 28; in some embodiments, at least 29; in some embodiments, at least 30; in some embodiments, at least 31; in some embodiments, at least 32; in some embodiments, at least 33; in some embodiments, at least 34; in some embodiments, at least 35) bases, optionally contiguous, of a base sequence that is identical or complementary to a base sequence of nucleic acid, e.g., a gene or a transcript (e.g., mRNA) thereof. In some embodiments, the base sequence of an oligonucleotide is or comprises a sequence that is complementary to a target sequence in a gene or a transcript thereof. In some embodiments, the sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60 or more nucleobases in length.

In some embodiments, a target sequence is or comprises a characteristic sequence of a nucleic acid sequence (e.g., of an gene or a transcript thereof) in that it defines the nucleic acid sequence over others in a relevant organism; for example, a characteristic sequence is not in or has at least various mismatches from other genomic nucleic acid sequences (e.g., genes) or transcripts thereof in a relevant organism. In some embodiments, a characteristic sequence of a transcript defines that transcript over other transcripts in a relevant organism; for example, in some embodiments, a characteristic sequence is not in transcripts that are transcribed from a different nucleic acid sequence (e.g., a different gene). In some embodiments, transcript variants from a nucleic acid sequence (e.g., mRNA variants of a gene) may share a common characteristic sequence that defines them from, e.g., transcripts of other genes. In some embodiments, a characteristic sequence comprises a target adenosine. In some embodiments, an oligonucleotide selectively forms a duplex with a nucleic acid comprising a target adenosine, wherein the target adenosine is within the duplex region and can be modified by a protein such as ADAR1 or ADAR2.

Base sequences of provided oligonucleotides, as appreciated by those skilled in the art, typically have sufficient lengths and complementarity to their target nucleic acids, e.g., RNA transcripts (e.g., pre-mRNA, mature mRNA, etc.) for, e.g., site-directed editing of target adenosines. In some embodiments, an oligonucleotide is complementary to a portion of a target RNA sequence comprising a target adenosine (as appreciated by those skilled in the art, in many instances target nucleic acids are longer than oligonucleotides of the present disclosure, and complementarity may be properly assessed based on the shorter of the two, oligonucleotides). In some embodiments, the base sequence of an oligonucleotide has 90% or more identity with the base sequence of an oligonucleotide disclosed in a Table, wherein each T can be independently substituted with U and vice versa. In some embodiments, the base sequence of an oligonucleotide has 95% or more identity with the base sequence of an oligonucleotide disclosed in a Table, wherein each T can be independently substituted with U and vice versa. In some embodiments, the base sequence of an oligonucleotide comprises a continuous span of 15, 16, 17, 18, 19, 20, 21, 22, 23, 24, 25, 26, 27, 28, 29, 30, 31, 32, 33, 34, 35, 36, 37, 38, 39, 40 or more bases of an oligonucleotide disclosed in a Table, wherein each T can be independently substituted with U and vice versa, except that one or more bases within the span are abasic (e.g., a nucleobase is absent from a nucleotide).

In some embodiments, the present disclosure pertains to an oligonucleotide having a base sequence which comprises the base sequence of any oligonucleotide disclosed herein, wherein each T may be independently replaced with U and vice versa.

In some embodiments, the present disclosure pertains to an oligonucleotide having a base sequence which is the base sequence of any oligonucleotide disclosed herein, wherein each T may be independently replaced with U and vice versa.

In some embodiments, the present disclosure pertains to an oligonucleotide having a base sequence which comprises at least 15 contiguous bases of the base sequence of any oligonucleotide disclosed herein, wherein each T may be independently replaced with U and vice versa.

In some embodiments, the present disclosure pertains to an oligonucleotide having a base sequence which is at least 90% identical to the base sequence of any oligonucleotide disclosed herein, wherein each T may be independently replaced with U and vice versa.

In some embodiments, the present disclosure pertains to an oligonucleotide having a base sequence which is at least 95% identical to the base sequence of any oligonucleotide disclosed herein, wherein each T may be independently replaced with U and vice versa.

In some embodiments, a base sequence of an oligonucleotide is, comprises, or comprises 10-40, e.g., 15, 16, 17, 18, 19, 20, 21, 22, 23, 24, 25, 26, 27, 28, 29, 30, 31, 32, 33, 34, 35, 36, 37, 38, 39, 40 contiguous bases of the base sequence of any oligonucleotide describer herein, wherein each T may be independently replaced with U and vice versa.

In some embodiments, an oligonucleotide is an oligonucleotide presented in a Table herein.

In some embodiments, the base sequence of an oligonucleotide is complementary to that of a target nucleic acid, e.g., a portion comprising a target adenosine.

In some embodiments, an oligonucleotide has a base sequence which comprises at least 15 contiguous bases (e.g., 15, 16, 17, 18, 19, or 20) of an oligonucleotide in a Table, wherein each T can be independently substituted with U and vice versa.

In some embodiments, an oligonucleotide comprises a base sequence or portion thereof (e.g., a portion comprising 10-40, e.g., 15, 16, 17, 18, 19, 20, 21, 22, 23, 24, 25, 26, 27, 28, 29, 30, 31, 32, 33, 34, 35, 36, 37, 38, 39, 40 nucleobases) described in any of the Tables, wherein each T may be independently replaced with U and vice versa, and/or a sugar, nucleobase, and/or internucleotidic linkage modification and/or stereochemistry, and/or a pattern thereof described in any of the Tables, and/or an additional chemical moiety (in addition to an oligonucleotide chain, e.g., a target moiety, a lipid moiety, a carbohydrate moiety, etc.) described in any of the Tables.

In some embodiments, the terms “complementary,” “fully complementary” and “substantially complementary” may be used with respect to the base matching between an oligonucleotide and a target sequence, as will be understood by those skilled in the art from the context of their uses. It is noted that substitution of T for U, or vice versa, generally does not alter the amount of complementarity. As used herein, an oligonucleotide that is “substantially complementary” to a target sequence is largely or mostly complementary but not necessarily 100% complementary. In some embodiments, a sequence (e.g., an oligonucleotide) which is substantially complementary has one or more, e.g., 1, 2, 3, 4 or 5 mismatches when maximally aligned to its target sequence. In some embodiments, an oligonucleotide has a base sequence which is substantially complementary to a target sequence of a target nucleic acid. In some embodiments, an oligonucleotide has a base sequence which is substantially complementary to the complement of the sequence of an oligonucleotide disclosed herein. As appreciated by those skilled in the art, in some embodiments, sequences of oligonucleotides need not be 100% complementary to their targets for oligonucleotides to perform their functions (e.g., converting A to I in a nucleic acid. In some embodiments, a mismatch is well tolerated at the 5′ and/or 3′ end or the middle of an oligonucleotide. In some embodiments, one or more mismatches are preferred for adenosine modification as demonstrated herein. In some embodiments, oligonucleotides comprise portions for complementarity to target nucleic acids, and optionally portions that are not primilary for complementarity to target nucleic acids; for example, in some embodiments, oligonucleotides may comprise portions for protein binding. In some embodiments, base sequences of provided oligonucleotides are fully complementary to their target sequences (A-T/U and C-G base pairing). In some embodiments, base sequences of provided oligonucleotides are fully complementary to their target sequences (A-T/U and C-G base pairing) except at a nucleoside opposite to a target nucleoside (e.g., adenosine).

In some embodiments, the present disclosure provides an oligonucleotide comprising a sequence found in an oligonucleotide described in a Table, wherein one or more U is independently and optionally replaced with T or vice versa. In some embodiments, an oligonucleotide can comprise at least one T and/or at least one U. In some embodiments, the present disclosure provides an oligonucleotide comprising a sequence found in an oligonucleotide described in a Table herein, wherein the said sequence has over 50% identity with the sequence of the oligonucleotide described in a Table. In some embodiments, the present disclosure provides an oligonucleotide whose base sequence is the sequence of an oligonucleotide disclosed in a Table, wherein each T may be independently replaced with U and vice versa. In some embodiments, the present disclosure provides an oligonucleotide comprising a sequence found in an oligonucleotide in a Table, wherein the oligonucleotides have a pattern of backbone linkages, pattern of backbone chiral centers, and/or pattern of backbone phosphorus modifications of the same oligonucleotide or another oligonucleotide in a Table herein.

In some embodiments, the disclosure provides an oligonucleotide having a base sequence which is, comprises, or comprises a portion of the base sequence of an oligonucleotide disclosed herein, e.g., in a Table, wherein each T may be independently replaced with U and vice versa, wherein the oligonucleotide optionally further comprises a chemical modification, stereochemistry, format, an additional chemical moiety described herein (e.g., a targeting moiety, lipid moiety, carbohydrate moiety, etc.), and/or another structural feature.

In some embodiments, a “portion” (e.g., of a base sequence or a pattern of modifications or other structural element) is at least 5, 6, 7, 8, 9, 10, 11, 12, 13, 14, 15, 16, 17, 18, 19, or 20 monomeric units long.

Those skilled in the art reading the present disclosure will appreciate that technologies herein may be utilized to target various target nucleic acids comprising target adenosine for editing. In some embodiments, a target nucleic acid is a transcript of a PiZZ allele. In some embodiments, a target adenosine is . . . atcgacAagaaagggactgaagc . . . . In some embodiments, oligonucleotides of the present disclosure have suitable base sequences so that they have sufficient complementarity to selectively form duplexes with a portion of a transcript that comprise the target adenosine for editing.

As described herein, nucleosides opposite to target nucleosides (e.g., A) can be positioned at various locations. In some embodiments, an opposite nucleoside is at position 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more from the 5′-end of an oligonucleotide. In some embodiments, it is at position 3 or more from the 5′-end of an oligonucleotide. In some embodiments, it is at position 4 or more from the 5′-end of an oligonucleotide. In some embodiments, it is at position 5 or more from the 5′-end of an oligonucleotide. In some embodiments, it is at position 6 or more from the 5′-end of an oligonucleotide. In some embodiments, it is at position 7 or more from the 5′-end of an oligonucleotide. In some embodiments, it is at position 8 or more from the 5′-end of an oligonucleotide. In some embodiments, it is at position 9 or more from the 5′-end of an oligonucleotide. In some embodiments, it is at position 10 or more from the 5′-end of an oligonucleotide. In some embodiments, an opposite nucleoside is at position 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more from the 3′-end of an oligonucleotide. In some embodiments, it is at position 3 or more from the 3′-end of an oligonucleotide. In some embodiments, it is at position 4 or more from the 3′-end of an oligonucleotide. In some embodiments, it is at position 5 or more from the 3′-end of an oligonucleotide. In some embodiments, it is at position 6 or more from the 3′-end of an oligonucleotide. In some embodiments, it is at position 7 or more from the 3′-end of an oligonucleotide. In some embodiments, it is at position 8 or more from the 3′-end of an oligonucleotide. In some embodiments, it is at position 9 or more from the 3′-end of an oligonucleotide. In some embodiments, it is at position 10 or more from the 3′-end of an oligonucleotide. In some embodiments, nucleobases at position 1 from the 5′-end and/or the 3′-end are complementary to corresponding nucleobases in target sequences when aligned for maximum complementarity. In some embodiments, certain positions, e.g., position 6, 7, or 8, may provide higher editing efficiency.

As examples, certain oligonucleotides comprising certain example base sequences, nucleobase modifications and patterns thereof, sugar modifications and patterns thereof, internucleotidic linkages and patterns thereof, linkage phosphorus stereochemistry and patterns thereof, linkers, and/or additional chemical moieties, etc., are presented in Table 1, below. Among other things, these oligonucleotides may be utilized to correct a G to A mutation in a gene or gene product (e.g., by converting A to I). In some embodiments, listed in Tables are stereorandom oligonucleotide compositions. In some embodiments, the present disclosure provides chirally controlled oligonucleotide compositions.

Table 1. Example oligonucleotides and/or compositions.

TABLE 1A
Example oligonucleotides and/or compositions that target ACTB.

| | SEQ | Base | | Stereochemistry/ |
|---|---|---|---|---|
| ID | ID NO | Sequence | Description | Linkage |
| WV- | 1 | UACAUAAUUUAG | mU*mA*mC*rA*rU*rA*rA*rU*rU*rU*rA*rG*rA*rC | XXXXX XXXXX |
| 23388 | | ACGUAAGCAAUG | *rG*rU*rA*rA*rG*rC*rA*rA*rU*rG*rC*rC*rA*mU | XXXXX XXXXX |
| | | CCAUCA | *mC*mA | XXXXX XXXX |
| WV- | 2 | ACAUAAUUUAGA | fA*fC*fA*fU*fA*fA*fU*fU*fU*fA*fG*fA*fC*fG* | XXXXX XXXXX |
| 23928 | | CGUAAGCAAUGC | fU*mA*mA*mG*mC*mA*mA*mU*mG*C*C*A*mU | XXXXX XXXXX |
| | | CAUCAC | *mC*mA*mC | XXXXX XXXX |
| WV- | 3 | ACAUAAUUUAGA | fA*fC*fA*fU*fA*fA*fU*fU*fU*fA*fG*fA*fC*fG* | XXXXX XXXXX |
| 23930 | | CGUAAGCAAUGC | fU*mA*mA*mG*mC*mA*mA*mU*mG*fC*C*A*mU | XXXXX XXXXX |
| | | CAUCAC | *mC*mA*mC | XXXXX XXXX |
| WV- | 4 | UACAUAAUUUAG | fU*SfA*SfC*SfA*SfU*SfA*SfA*SfU*SfU*SfU*SfA* | SSSSS SSSSS SSSSS |
| 27385 | | ACGUAAGCAAUG | SfG*SfA*SfC*SfG*SfU*SmA*SmA*SmG*SmC*SmA* | SSSSS SSSSS SSSSS S |
| | | CCAUCACC | SmA*SmU*SmG*SC*SC*SA*SmU*SmC*SmA*SmC* | |
| | | | SmC | |
| WV- | 5 | UACAUAAUUUAG | fU*SfA*SfC*SfA*SfU*SfA*SfA*SfU*SfU*SfU*SfA* | SSSSS SSSSS SSSSS |
| 27386 | | ACGAAAGCAAUG | SfC*SfA*SfC*SfG*SfA*SmA*SmA*SmG*SmC*SmA* | SSSSS SSSSS SSSSS S |
| | | CCAUCACC | SmA*SmU*SmG*SC*SC*SA*SmU*SmC*SmA*SmC* | |
| | | | SmC | |
| WV- | 6 | ACAUAAUUUAGA | fA*SfC*SfA*SfU*SfA*SfA*SfU*SfU*SfU*SfA*SfG* | SSSSS SSSSS SSSSS |
| 27387 | | CGUAAGCAAUGC | SfA*SfC*SfG*SfU*SmA*SmA*SmG*SmC*SmA*SmA* | SSSSS SSSSS SSSS |
| | | CAUCAC | SmU*SmG*SC*SC*SA*SmU*SmC*SmA*SmC | |
| WV- | 7 | ACAUAAUUUAGA | fA*SfC*SfA*SfU*SfA*SfA*SfU*SfU*SfU*SfA*SfC* | SSSSS SSSSS SSSSS |
| 27388 | | CGAAAGCAAUGC | SfA*SfC*SfG*SfA*SmA*SmA*SmG*SmC*SmA*SmA* | SSSSS SSSSS SSSS |
| | | CAUCAC | SmU*SmG*SC*SC*SA*SmU*SmC*SmA*SmC | |
| WV- | 8 | AUAAUUUAGACG | fA*SfU*SfA*SfA*SfU*SfU*SfU*SfA*SfG*SfA*SfC* | SSSSS SSSSS SSSSS |
| 27389 | | UAAGCAAUGCCA | SfG*SfU*SmA*SmA*SmG*SmC*SmA*SmA*SmU*SmG | SSSSS SSSSS SS |
| | | UCAC | *SC*SC*SA*SmU*SmC*SmA*SmC | |
| WV- | 9 | AUAAUUUACACG | fA*SfU*SfA*SfA*SfU*SfU*SfU*SfA*SfC*SfA*SfC* | SSSSS SSSSS SSSSS |
| 27390 | | AAAGCAAUGCCA | SfG*SfA*SmA*SmA*SmG*SmC*SmA*SmA*SmU*SmG | SSSSS SSSSS SS |
| | | UCAC | *SC*SC*SA*SmU*SmC*SmA*SmC | |
| WV- | 10 | AUAAUUUAGACG | fA*SfU*SfA*SfA*SfU*SfU*SfU*SfA*SfG*SfA*SfC* | SSSSS SSSSS SSSSS |
| 27391 | | UAAGCAAUGCCA | SfG*SfU*SmA*SmA*SmG*SmC*SmA*SmA*SmU*SmG | SSSSS SSSSS S |
| | | UCA | *SC*SC*SA*SmU*SmC*SmA | |
| WV- | 11 | AUAAUUUACACG | fA*SfU*SfA*SfA*SfU*SfU*SfU*SfA*SfC*SfA*SfC* | SSSSS SSSSS SSSSS |
| 27392 | | AAAGCAAUGCCA | SfG*SfA*SmA*SmA*SmG*SmC*SmA*SmA*SmU*SmG | SSSSS SSSSS S |
| | | UCA | *SC*SC*SA*SmU*SmC*SmA | |
| WV- | 12 | UACAUAAUUUAC | fU*SfA*SfC*SfA*SfU*SfA*SfA*SfU*SfU*SfU*SfA* | SSSSS SSSSS SSSSS |
| 27394 | | ACGAAAGCAAUG | SfC*SfA*SfC*SfG*SfA*SmA*SmA*SmG*SmC*SmA* | SSSSS SSSSS RRSSSS |
| | | CCAUCACC | SmA*SmU*SmG*SC*SC*RA*RmU*SmC*SmA*SmC* | |
| | | | SmC | |
| WV- | 13 | ACAUAAUUUAGA | fA*SfC*SfA*SfU*SfA*SfA*SfU*SfU*SfU*SfA*SfG* | SSSSS SSSSS SSSSS |
| 27395 | | CGUAAGCAAUGC | SfA*SfC*SfG*SfU*SmA*SmA*SmG*SmC*SmA*SmA* | SSSSS SSSSRRSSS |
| | | CAUCAC | SmU*SmG*SC*SC*RA*RmU*SmC*SmA*SmC | |
| WV- | 14 | UACAUAAUUUAC | fU*SfA*SfC*SfA*SfU*SfA*SfA*SfU*SfU*SfU*SfA* | SSSSS SSSSS SSSSS |
| 27402 | | ACGAAAGCAAUG | SfC*SfA*SfC*SfG*SfA*SmA*SmA*SmG*SmC*SmA* | SSSSS SSSSS SSSSS S |
| | | CCAUCACC | SmA*SmU*SmG*SfC*SC*SA*SmU*SmC*SmA*SmC* | |
| | | | SmC | |
| WV- | 15 | ACAUAAUUUAGA | fA*SfC*SfA*SfU*SfA*SfA*SfU*SfU*SfU*SfA*SfG* | SSSSS SSSSS SSSSS |
| 27403 | | CGUAAGCAAUGC | SfA*SfC*SfG*SfU*SmA*SmA*SmG*SmC*SmA*SmA* | SSSSS SSSSS SSSS |
| | | CAUCAC | SmU*SmG*SfC*SC*SA*SmU*SmC*SmA*SmC | |
| WV- | 16 | UACAUAAUUUAC | fU*SfA*SfC*SfA*SfU*SfA*SfA*SfU*SfU*SfU*SfA* | SSSSS SSSSS SSSSS |
| 27410 | | ACGAAAGCAAUG | SfC*SfA*SfC*SfG*SfA*SmA*SmA*SmG*SmC*SmA* | SSSSS SSSSS RRSSSS |
| | | CCAUCACC | SmA*SmU*SmG*SfC*SC*RA*RmU*SmC*SmA*SmC* | |
| | | | SmC | |
| WV- | 17 | ACAUAAUUUAGA | fA*SfC*SfA*SfU*SfA*SfA*SfU*SfU*SfU*SfA*SfG* | SSSSS SSSSS SSSSS |
| 27411 | | CGUAAGCAAUGC | SfA*SfC*SfG*SfU*SmA*SmA*SmG*SmC*SmA*SmA* | SSSSS SSSSRRSSS |
| | | CAUCAC | SmU*SmG*SfC*SC*RA*RmU*SmC*SmA*SmC | |
| WV- | 18 | UACAUAAUUUAG | Mod001L001fU*SfA*SfC*SfA*SfU*SfA*SfA*SfU*SfU* | OSSSS SSSSS SSSSS |
| 27457 | | ACGUAAGCAAUG | SfU*SfA*SfG*SfA*SfC*SfG*SfU*SmA*SmA*SmG* | SSSSS SSSSS SSSSS |
| | | CCAUCACC | SmC*SmA*SmA*SmU*SmG*SC*SC*SA*SmU*SmC* | SS |
| | | | SmA*SmC*SmC | |
| WV- | 19 | ACAUAAUUUAGA | Mod001U001fA*SfC*SfA*SfU*SfA*SfA*SfU*SfU*SfU* | OSSSS SSSSS SSSSS |
| 27458 | | CGUAAGCAAUGC | SfA*SfG*SfA*SfC*SfG*SfU*SmA*SmA*SmG*SmC* | SSSSS SSSSS SSSSS |
| | | CAUCAC | SmA*SmA*SmU*SmG*SC*SC*SA*SmU*SmC*SmA* | |
| | | | SmC | |
| WV- | 20 | UACAUAAUUUAG | Mod001U001fU*SfA*SfC*SfA*SfU*SfA*SfA*SfU*SfU* | OSSSS SSSSS SSSSS |
| 27459 | | ACGUAAGCAAUG | SfU*SfA*SfG*SfA*SfC*SfG*SfU*SmA*SmA*SmG* | SSSSS SSSSS SSSSS |
| | | CCAUCACC | SmC*SmA*SmA*SmU*SmG*SfC*SC*SA*SmU*SmC* | SS |
| | | | SmA*SmC*SmC | |
| WV- | 21 | ACAUAAUUUAGA | Mod001U001fA*SfC*SfA*SfU*SfA*SfA*SfU*SfU*SfU* | OSSSS SSSSS SSSSS |
| 27460 | | CGUAAGCAAUGC | SfA*SfG*SfA*SfC*SfG*SfU*SmA*SmA*SmG*SmC* | SSSSS SSSSS SSSSS |
| | | CAUCAC | SmA*SmA*SmU*SmG*SfC*SC*SA*SmU*SmC*SmA* | |
| | | | SmC | |
| WV- | 22 | ACAUAAUUUAGA | fA*SfC*SfA*SfU*SfA*SfA*SfU*SfU*SfU*SfA*SfG* | SSSSS SSSSS SSSSS |
| 28787 | | CGUAAGCAAUGC | SfA*SfC*SfG*SfU*SmA*SmA*SmG*SmC*SmA*SmA* | SSSSS SSSSnX SSSS |
| | | UAUCAC | SmU*SmG*Sm51C*SUsm04n001A*SmU*SmC*SmA*SmC | |
| WV- | 23 | ACAUAAUUUAGA | fA*SfC*SfA*SfU*SfA*SfA*SfU*SfU*SfU*SfA*SfG* | SSSSS SSSSS SSSSS |
| 28788 | | CGUAAGCAAUGC | SfA*SfC*SfG*SfU*SmA*SmA*SmG*SmC*SmA*SmA* | SSSSS SSSSO SSSS |
| | | UAUCAC | SmU*SmG*SC*SUsmO4A*SmU*SmC*SmA*SmC | |
| WV- | 24 | ACAUAAUUUAGA | fA*SfC*SfA*SfU*SfA*SfA*SfU*SfU*SfU*SfA*SfG* | SSSSS SSSSS SSSSS |
| 28789 | | CGUAAGCAAUGC | SfA*SfC*SfG*SfU*SmA*SmA*SmG*SmC*SmA*SmA* | SSSSS SSSSnX SSSS |
| | | UAUCAC | SmU*SmG*SC*SUsm04n001A*SmU*SmC*SmA*SmC | |
| WV- | 25 | ACAUAAUUUAGA | fA*SfC*SfA*SfU*SfA*SfA*SfU*SfU*SfU*SfA*SfG* | SSSSS SSSSS SSSSS |
| 28790 | | CGUAAGCAAUGC | SfA*SfC*SfG*SfU*SmA*SmA*SmG*SmC*SmA*SmA* | SSSSS SSSSO SSSS |
| | | UAUCAC | SmU*SmG*SfC*SUsmO4A*SmU*SmC*SmA*SmC | |
| WV- | 26 | ACAUAAUUUAGA | fA*SfC*SfA*SfU*SfA*SfA*SfU*SfU*SfU*SfA*SfG* | SSSSS SSSSS SSSSS |
| 28791 | | CGUAAGCAAUGC | SfA*SfC*SfG*SfU*SmA*SmA*SmG*SmC*SmA*SmA* | SSSSS SSSSnX SSSS |
| | | UAUCAC | SmU*SmG*SfC*SUsm04n001A*SmU*SmC*SmA*SmC | |
| WV- | 27 | ACAUAAUUUAGA | fA*SfC*SfA*SfU*SfA*SfA*SfU*SfU*SfU*SfA*SfG* | SSSSS SSSSS SSSSS |
| 31967 | | CGUAAGCAAUGC | SfA*SfC*SfG*SfU*SmA*SmA*SmG*SmC*SmA*SmA* | SSSSS SSSSS nXSSnX |
| | | CAUCAC | SmU*SmG*SC*SC*SAn001mU*SmC*SmAn001mC | |
| WV- | 28 | ACAUAAUUUAGA | fA*SfC*SfA*SfU*SfA*SfA*SfU*SfU*SfU*SfA*SfG* | SSSSS SSSSS SSSSS |
| 31968 | | CGUAAGCAAUGC | SfA*SfC*SfG*SfU*SmA*SmA*SmG*SmC*SmAn001mA* | SSSSnX SSSSS |
| | | CAUCAC | SmU*SmG*SC*SC*SAn001mU*SmC*SmAn001mC | nXSSnX |
| WV- | 29 | ACAUAAUUUAGA | fA*SfC*SfAn001fU*SfA*SfA*SfU*SfU*SfU*SfA* | SSnXSS SSSSS |
| 31969 | | CGUAAGCAAUGC | SfGn001fA*SfC*SfGn001fU*SmA*SmA*SmG*SmC*SmA* | nXSSnXS SSSSS |
| | | CAUCAC | SmA*SmU*SmG*SC*SC*SA*SmU*SmC*SmA*SmC | SSSSS SSSS |
| WV- | 30 | ACAUAAUUUAGA | fA*SfC*SfAn001fU*SfA*SfA*SfU*SfU*SfU*SfA* | SSnXSS SSSSS |
| 31970 | | CGUAAGCAAUGC | SfGn001fA*SfC*SfG*SfU*SmA*SmA*SmG*SmC* | nXSSSS SSSSnX |
| | | CAUCAC | SmAn001mA*SmU*SmG*SC*SC*SA*SmU*SmC*SmA* | SSSSS SSSS |
| | | | SmC | |
| WV- | 31 | ACAUAAUUUAGA | fA*SfC*SfAn001fU*SfA*SfA*SfU*SfU*SfU*SfA*SfG* | SSnXSS SSSSS |
| 31971 | | CGUAAGCAAUGC | SfAn001fC*SfG*SfU*SmA*SmA*SmG*SmC*SmAn001mA | SnXSSS SSSSnX |
| | | CAUCAC | *SmU*SmG*SC*SC*SA*SmU*SmC*SmA*SmC | SSSSS SSSS |
| WV- | 32 | ACAUAAUUUAGA | fA*SfC*SfAn001fU*SfA*SfA*SfU*SfU*SfU*SfA*SfG* | SSnXSS SSSSS |
| 31972 | | CGUAAGCAAUGC | SfAn001fC*SfG*SfU*SmA*SmA*SmG*SmC*SmA*SmA* | SnXSSS SSSSS SSSSS |
| | | CAUCAC | SmU*SmG*SC*SC*SAn001mU*SmC*SmA*SmC | nXSSS |
| WV- | 33 | ACAUAAUUUAGA | fA*SfC*SfAn001fU*SfA*SfA*SfU*SfU*SfU*SfA*SfG* | SSnXSS SSSSS |
| 31973 | | CGUAAGCAAUGC | SfAn001fC*SfG*SfU*SmA*SmA*SmG*SmC*SmAn001mA | SnXSSS SSSSnX |
| | | CAUCAC | *SmU*SmG*SC*SC*SAn001mU*SmC*SmA*SmC | SSSSS nXSSS |
| WV- | 34 | ACAUAAUUUAGA | fA*SfC*SfA*SfU*SfA*SfA*SfU*SfU*SfU*SfA*SfG* | SSSSS SSSSS SnXSSS |
| 31974 | | CGUAAGCAAUGC | SfAn001fC*SfG*SfU*SmA*SmA*SmG*SmC*SmAn001mA | SSSSnX SSSSS nXSSS |
| | | CAUCAC | *SmU*SmG*SC*SC*SAn001mU*SmC*SmA*SmC | |
| WV- | 35 | ACAUAAUUUAGA | fA*SfC*SfA*SfU*SfA*SfA*SfU*SfU*SfU*SfA*SfG* | SSSSS SSSSS SSSSS |
| 31975 | | CGUAAGCAAUGC | SfA*SfC*SfG*SfU*SmA*SmA*SmG*SmC*SmA*SmA* | SSSSS SSSSS nXSSS |
| | | CAUCAC | SmU*SmG*SC*SC*SAn001mU*SmC*SmA*SmC | |
| WV- | 36 | ACAUAAUUUAGA | fA*SfC*SfA*SfU*SfA*SfA*SfU*SfU*SfU*SfA*SfG* | SSSSS SSSSS SSSSS |
| 31976 | | CGUAAGCAAUGC | SfA*SfC*SfG*SfU*SmA*SmA*SmG*SmC*SmAn001mA* | SSSSnX SSSSS SSSS |
| | | CAUCAC | SmU*SmG*SC*SC*SA*SmU*SmC*SmA*SmC | |
| WV- | 37 | ACAUAAUUUAGA | fA*SfC*SfAn001fU*SfA*SfA*SfU*SfU*SfU*SfA*SfG* | SSnXSS SSSSS SSSSS |
| 31977 | | CGUAAGCAAUGC | SfA*SfC*SfG*SfU*SmA*SmA*SmG*SmC*SmA*SmA* | S SSSSS SSSSS SSS |
| | | CAUCAC | SmU*SmG*SC*SC*SA*SmU*SmC*SmA*SmC | |
| WV- | 38 | ACAUAAUUUAGA | fA*SfC*SfA*SfU*SfA*SfA*SfU*SfU*SfU*SfA*SfG* | SSSSS SSSSS SnXSSS |
| 31978 | | CGUAAGCAAUGC | SfAn001fC*SfG*SfU*SmA*SmA*SmG*SmC*SmA*SmA* | SSSSS SSSSS SSSS |
| | | CAUCAC | SmU*SmG*SC*SC*SA*SmU*SmC*SmA*SmC | |
| WV- | 39 | ACAUAAUUUAGA | fA*SfC*SfA*SfU*SfA*SfA*SfU*SfU*SfU*SfA*SfG* | SSSSS SSSSS SSSSS |
| 31979 | | CGUAAGCAAUGC | SfA*SfC*SfG*SfU*SmA*SmA*SmG*SmC*SmA*SmA* | SSSSS SSSSS SSSnX |
| | | CAUCAC | SmU*SmG*SC*SC*SA*SmU*SmC*SmAn001mC | |
| WV- | 40 | ACAUAAUUUAGA | fA*SfC*SfA*SfU*SfA*SfA*SfU*SfU*SfU*SfA*SfG* | SSSSS SSSSS SSSnXS |
| 31980 | | CGUAAGCAAUGC | SfA*SfC*SfGn001fU*SmA*SmA*SmG*SmC*SmAn001mA | SSSSnX SSSSS SSSS |
| | | CAUCAC | *SmU*SmG*SC*SC*SA*SmU*SmC*SmA*SmC | |
| WV- | 41 | ACAUAAUUUAGA | fA*SfC*SfA*SfU*SfA*SfA*SfU*SfU*SfU*SfA*SfG* | SSSSS SSSSS SSSSS |
| 31981 | | CGUAAGCAAUGC | SfA*SfC*SfG*SfU*SmA*SmA*SmG*SmC*SmAn001mA* | SSSSnX SSSSS nXSSS |
| | | CAUCAC | SmU*SmG*SC*SC*SAn001mU*SmC*SmA*SmC | |
| WV- | 42 | ACAUAAUUUAGA | fA*SfC*SfAn001fU*SfA*SfA*SfU*SfU*SfU*SfA*SfG* | SSnXSS SSSSS SSSSS |
| 31982 | | CGUAAGCAAUGC | SfA*SfC*SfG*SfU*SmA*SmA*SmG*SmC*SmA*SmA* | SSSSS SSSSS nXSSS |
| | | CAUCAC | SmU*SmG*SC*SC*SAn001mU*SmC*SmA*SmC | |
| WV- | 43 | ACAUAAUUUAGA | fA*SfC*SfA*SfU*SfA*SfA*SfU*SfU*SfU*SfA* | SSSSS SSSSS |
| 31983 | | CGUAAGCAAUGC | SfGn001fA*SfC*SfGn001fU*SmA*SmA*SmG*SmC*SmA* | nXSSnXS SSSSS |
| | | CAUCAC | SmA*SmU*SmG*SC*SC*SA*SmU*SmC*SmA*SmC | SSSSS SSSS |
| WV- | 44 | ACAUAAUUUAGA | fA*SfC*SfAn001fU*SfA*SfA*SfU*SfU*SfU*SfA*SfG* | SSnXSS SSSSS |
| 31984 | | CGUAAGCAAUGC | SfA*SfC*SfGn001fU*SmA*SmA*SmG*SmC*SmA*SmA* | SSSnXS SSSSS SSSSS |
| | | CAUCAC | SmU*SmG*SC*SC*SA*SmU*SmC*SmA*SmC | SSSS |
| WV- | 45 | ACAUAAUUUAGA | fA*SfC*SfAn001fU*SfA*SfA*SfU*SfU*SfU*SfA* | SSnXSS SSSSS |
| 31985 | | CGUAAGCAAUGC | SfGn001fA*SfC*SfG*SfU*SmA*SmA*SmG*SmC*SmA* | nXSSSS SSSSS SSSSS |
| | | CAUCAC | SmA*SmU*SmG*SC*SC*SA*SmU*SmC*SmA*SmC | SSSS |
| WV- | 46 | ACAUAAUUUAGA | fA*SfC*SfA*SfU*SfA*SfA*SfU*SfU*SfU*SfA* | SSSSS SSSSS nXSSSS |
| 31986 | | CGUAAGCAAUGC | SfGn001fA*SfC*SfG*SfU*SmA*SmA*SmG*SmC* | SSSSnX SSSSS SSSS |
| | | CAUCAC | SmAn001mA*SmU*SmG*SC*SC*SA*SmU*SmC*SmA* | |
| | | | SmC | |
| WV- | 47 | ACAUAAUUUAGA | fA*SfC*SfAn001fU*SfA*SfA*SfU*SfU*SfU*SfA*SfG* | SSnXSS SSSSS SSSSS |
| 31987 | | CGUAAGCAAUGC | SfA*SfC*SfG*SfU*SmA*SmA*SmG*SmC*SmAn001mA* | SSSSnX SSSSS SSSS |
| | | CAUCAC | SmU*SmG*SC*SC*SA*SmU*SmC*SmA*SmC | |
| WV- | 48 | ACAUAAUUUAGA | Mod001L001fA*SfC*SfA*SfU*SfA*SfA*SfU*SfU*SfU* | OSSSSSSSSSSSSSSSS |
| 32101 | | CAAAGGCAAUGC | SfA*SfC*SfA*SfC*SfA*SfA*SmA*SmG*SmG*SmC* | SSSnXSSSSSnXSSnX |
| | | CAUCAC | SmAn001mA*SmU*SmG*SC*SC*SAn001mU*SmC*SmAn001mC | |
| WV- | 49 | ACAUAAUUUAGA | Mod001L001fAn001fC*SfA*SfU*SfA*SfA*SfU*SfU*SfU* | OnXSSSSSSSSSSSSnX |
| 35713 | | CAAAGGCAAUGC | SfA*SfC*SfA*SfC*SfAn001fA*SmAn001mG*SmG*SmC*SmA* | SnXSSSSSSSSSnXSSn |
| | | CAUCAC | SmA*SmU*SmG*SC*SC*SAn001mU*SmC*SmAn001mC | X |
| WV- | 50 | ACAUAAUUUAGA | Mod001L001fAn001fC*SfA*SfU*SfA*SfA*SfU*SfU*SfU* | OnXSSSSSSSSSSSSSS |
| 35736 | | CGAAAGCAAUGC | SfA*SfC*SfA*SfC*SfG*SfA*SmA*SmA*SmG*SmC* | SSSSnXSSSSSnXSSnX |
| | | CAUCAC | SmAn001mA*SmU*SmG*SfC*SC*SAn001mU*SmC*SmAn001mC | |
| WV- | 51 | ACAUAAUUUAGA | Mod001L001fAn001fC*SfA*SfU*SfA*SfA*SfU*SfU*SfU* | OnXSSSSSSSSSSSSnX |
| 35737 | | CGAAAGCAAUGC | SfA*SfC*SfA*SfC*SfGn001fA*SmAn001mA*SmG*SmC*SmA* | SnXSSSSSSSSSnXSSn |
| | | CAUCAC | SmA*SmU*SmG*SfC*SC*SAn001mU*SmC*SmAn001mC | X |
| WV- | 52 | ACAUAAUUUAGA | Mod001L001fAn001RfC*SfA*SfU*SfA*SfA*SfU*SfU*SfU* | OnRSSSSSSSSSSSSnR |
| 37314 | | CGAAAGCAAUGC | SfA*SfC*SfA*SfC*SfGn001RfA*SmAn001RmA*SmG*SmC* | SnRSSSSSSSSSnRSSn |
| | | CAUCAC | SmA*SmA*SmU*SmG*SfC*SC*SAn001RmU*SmC*SmAn001RmC | R |
| WV- | 53 | ACAUAAUUUAGA | Mod001L001fAn001SfC*SfA*SfU*SfA*SfA*SfU*SfU*SfU* | OnSSSSSSSSSSSSSnSS |
| 37315 | | CGAAAGCAAUGC | SfA*SfC*SfA*SfC*SfGn001SfA*SmAn001SmA*SmG*SmC* | nSSSSSSSSSSnSSSnS |
| | | CAUCAC | SmA*SmA*SmU*SmG*SfC*SC*SAn001SmU*SmC*SmAn001SmC | |
| WV- | 54 | ACAUAAUUUAGA | Mod001L001mAn001mC*SmA*SfU*SfA*SfA*SfU*SfU*SfU* | OnXSSSSSSSSSSSSnX |
| 37326 | | CGAAAGCAAUGC | SfA*SfC*SfA*SfC*SfGn001fA*SmAn001mA*SmG*SmC*SmA* | SnXSSSSSSSSSnXSSn |
| | | CAUCAC | SmA*SmU*SmG*SfC*SC*SAn001mU*SmC*SmAn001mC | X |
| WV- | 55 | ACAUAAUUUAGA | Mod001L001mAn001RmC*SmA*SfU*SfA*SfA*SfU*SfU*SfU* | OnRSSSSSSSSSSSSnR |
| 37327 | | CGAAAGCAAUGC | SfA*SfC*SfA*SfC*SfGn001RfA*SmAn001RmA*SmG*SmC* | SnRSSSSSSSSSnRSSn |
| | | CAUCAC | SmA*SmA*SmU*SmG*SfC*SC*SAn001RmU*SmC*SmAn001RmC | R |
| WV- | 56 | ACAUAAUUUACA | Mod001L001mAn001SmC*SmA*SfU*SfA*SfA*SfU*SfU*SfU* | OnSSSSSSSSSSSSSnSS |
| 37328 | | CGAAAGCAAUGC | SfA*SfC*SfA*SfC*SfGn001SfA*SmAn001SmA*SmG*SmC* | nSSSSSSSSSSnSSSnS |
| | | CAUCAC | SmA*SmA*SmU*SmG*SfC*SC*SAn001SmU*SmC*SmAn001SmC | |
| WV- | 57 | ACAUAAUUUACA | Mod001L001Aeon001m5Ceo*SAeo*SfU*SfA*SfA*SfU*SfU* | OnXSSSSSSSSSSSSnX |
| 37329 | | CGAAAGCAAUGC | SfU*SfA*SfC*SfA*SfC*SfGn001fA*SmAn001mA*SmG*SmC* | SnXSSSSSSSSSnXSSn |
| | | CAUCAC | SmA*SmA*SmU*SmG*SfC*SC*SAn001mU*SmC*SmAn001mC | X |
| WV- | 58 | ACAUAAUUUACA | Mod001L001Aeon001Rm5Ceo*SAeo*SfU*SfA*SfA*SfU* | OnRSSSSSSSSSSSSnR |
| 37330 | | CGAAAGCAAUGC | SfU*SfU*SfA*SfC*SfA*SfC*SfGn001RfA*SmAn001RmA* | SnRSSSSSSSSSnRSSn |
| | | CAUCAC | SmG*SmC*SmA*SmA*SmU*SmG*SfC*SC*SAn001RmU*SmC* | R |
| | | | SmAn001RmC | |
| WV- | 59 | ACAUAAUUUACA | Mod001L001Aeon001Sm5Ceo*SAeo*SfU*SfA*SfA*SfU* | OnSSSSSSSSSSSSSnSS |
| 37331 | | CGAAAGCAAUGC | SfU*SfU*SfA*SfC*SfA*SfC*SfGn001SfA*SmAn001SmA* | nSSSSSSSSSSnSSSnS |
| | | CAUCAC | SmG*SmC*SmA*SmA*SmU*SmG*SfC*SC*SAn001SmU*SmC* | |
| | | | SmAn001SmC | |
| WV- | 60 | ACAUAAUUUACA | Mod001L001mAn001mC*SfA*SmU*SfA*SmA*SfU*SmU*SfU* | OnXSSSSSSSSSSSSnX |
| 37332 | | CGAAAGCAAUGC | SmA*SfC*SmA*SfC*SfGn001fA*SmAn001mA*SmG*SmC*SmA* | SnXSSSSSSSSSnXSSn |
| | | CAUCAC | SmA*SmU*SmG*SfC*SC*SAn001mU*SmC*SmAn001mC | X |
| WV- | 61 | ACAUAAUUUACA | Mod001L001mAn001RmC*SfA*SmU*SfA*SmA*SfU*SmU*SfU* | OnRSSSSSSSSSSSSnR |
| 37333 | | CGAAAGCAAUGC | SmA*SfC*SmA*SfC*SfGn001RfA*SmAn001RmA*SmG*SmC* | SnRSSSSSSSSSnRSSn |
| | | CAUCAC | SmA*SmA*SmU*SmG*SfC*SC*SAn001RmU*SmC*SmAn001RmC | R |
| WV- | 62 | ACAUAAUUUACA | Mod001L001mAn001SmC*SfA*SmU*SfA*SmA*SfU*SmU*SfU* | OnSSSSSSSSSSSSSnSS |
| 37334 | | CGAAAGCAAUGC | SmA*SfC*SmA*SfC*SfGn001SfA*SmAn001SmA*SmG*SmC* | nSSSSSSSSSSnSSSnS |
| | | CAUCAC | SmA*SmA*SmU*SmG*SfC*SC*SAn001SmU*SmC*SmAn001SmC | |

TABLE 1B
Example oligonucleotides and/or compositions that target LUC.

| | SEQ | Base | | Stereochemistry/ |
|---|---|---|---|---|
| ID | ID NO | Sequence | Description | Linkage |
| WV- | 63 | UCUCUUUCCAUGGAAG | mUmCmUrCrUrUrUrCrCrArUrGrGrArArGrGrU | OOOOO OOOOO OOOOO |
| 20666 | | GUUCUAAACCAUCCUG | rUrCrUrArArArCrCrArUrCmCmUmG | OOOOO OOOOO OOOOO O |
| WV- | 64 | UCUCUUUCCAUGGAAG | mU*mCmUrCrUrUrUrCrCrArUrGrGrArArGrG | XOOOO OOOOO OOOOOO |
| 20689 | | GUUCUAAACCAUCCUG | rUrUrCrUrArArArCrCrArUrCmCmUmG | OOOOOO OOOOOO OOO |
| WV- | 65 | UCUCUUUCCAUGGAAG | mU*mC*mUrCrUrUrUrCrCrArUrGrGrArArG | XXOOO OOOOO OOOOO |
| 20690 | | GUUCUAAACCAUCCUG | rGrUrUrCrUrArArArCrCrArUrCmCmUmG | OOOOO OOOOO OOOOO O |
| WV- | 66 | UCUCUUUCCAUGGAAG | mU*mC*mU*rCrUrUrUrCrCrArUrGrGrArA | XXXOO OOOOO OOOOO |
| 20691 | | GUUCUAAACCAUCCUG | rGrGrUrUrCrUrArArArCrCrArUrCmCmUmG | OOOOO OOOOO OOOOO O |
| WV- | 67 | UCUCUUUCCAUGGAAG | mU*mC*mU*rC*rUrUrUrCrCrArUrGrGrArA | XXXXO OOOOO OOOOOO |
| 20692 | | GUUCUAAACCAUCCUG | rGrGrUrUrCrUrArArArCrCrArUrCmCmUmG | OOOOOO OOOOOO OOO |
| WV- | 68 | UCUCUUUCCAUGGAAG | mU*mC*mU*rC*rU*rUrUrCrCrArUrGrGrA | XXXXX OOOOO OOOOO |
| 20693 | | GUUCUAAACCAUCCUG | rArGrGrUrUrCrUrArArArCrCrArUrCmCmUmG | OOOOO OOOOO OOOOO O |
| WV- | 69 | UCUCUUUCCAUGGAAG | mU*mC*mU*rC*rU*rU*rUrCrCrArUrGrGrA | XXXXX XOOOO OOOOO |
| 20694 | | GUUCUAAACCAUCCUG | rArGrGrUrUrCrUrArArArCrCrArUrCmCmUmG | OOOOO OOOOO OOOOO O |
| WV- | 70 | UCUCUUUCCAUGGAAG | mU*mC*mU*rC*rU*rU*rU*rCrCrArUrGrG | XXXXX XXOOO OOOOO |
| 20695 | | GUUCUAAACCAUCCUG | rArArGrGrUrUrCrUrArArArCrCrArU | OOOOO OOOOO OOOOO O |
| | | | rCmCmUmG | |
| WV- | 71 | UCUCUUUCCAUGGAAG | mU*mC*mU*rC*rU*rU*rU*rC*rCrArUrGrG | XXXXX XXXOO OOOOO |
| 20696 | | GUUCUAAACCAUCCUG | rArArGrGrUrUrCrUrArArArCrCrArU | OOOOO OOOOO OOOOO O |
| | | | rCmCmUmG | |
| WV- | 72 | UCUCUUUCCAUGGAAG | mU*mC*mU*rC*rU*rU*rU*rC*rC*rArUrG | XXXXX XXXXO OOOOO |
| 20697 | | GUUCUAAACCAUCCUG | rGrArArGrGrUrUrCrUrArArArCrCrArU | OOOOO OOOOO OOOOO O |
| | | | rCmCmUmG | |
| WV- | 73 | UCUCUUUCCAUGGAAG | mU*mC*mU*rC*rU*rU*rU*rC*rC*rA*rU | XXXXX XXXXX OOOOO |
| 20698 | | GUUCUAAACCAUCCUG | rGrGrArArGrGrUrUrCrUrArArArCrCrArU | OOOOO OOOOO OOOOO O |
| | | | rCmCmUmG | |
| WV- | 74 | UCUCUUUCCAUGGAAG | mU*mC*mU*rC*rU*rU*rU*rC*rC*rA*rU* | XXXXX XXXXX XOOOO |
| 20699 | | GUUCUAAACCAUCCUG | rGrGrArArGrGrUrUrCrUrArArArCrCrArU | OOOOO OOOOO OOOOO O |
| | | | rCmCmUmG | |
| WV- | 75 | UCUCUUUCCAUGGAAG | mU*mC*mU*rC*rU*rU*rU*rC*rC*rA*rU* | XXXXX XXXXX XXOOO |
| 20700 | | GUUCUAAACCAUCCUG | rG*rGrArArGrGrUrUrCrUrArArArCrCrArU | OOOOO OOOOO OOOOO O |
| | | | rCmCmUmG | |
| WV- | 76 | UCUCUUUCCAUGGAAG | mU*mC*mU*rC*rU*rU*rU*rC*rC*rA*rU* | XXXXX XXXXX XXXOO |
| 20701 | | GUUCUAAACCAUCCUG | rG*rG*rArArGrGrUrUrCrUrArArArCrCrArU | OOOOO OOOOO OOOOO O |
| | | | rCmCmUmG | |
| WV- | 77 | UCUCUUUCCAUGGAAG | mU*mC*mU*rC*rU*rU*rU*rC*rC*rA*rU* | XXXXX XXXXX XXXXO |
| 20702 | | GUUCUAAACCAUCCUG | rG*rG*rA*rArGrGrUrUrCrUrArArArCrCrA | OOOOO OOOOO OOOOO O |
| | | | rUrCmCmUmG | |
| WV- | 78 | UCUCUUUCCAUGGAAG | mU*mC*mU*rC*rU*rU*rU*rC*rC*rA*rU* | XXXXX XXXXX XXXXX |
| 20703 | | GUUCUAAACCAUCCUG | rG*rG*rA*rA*rGrGrUrUrCrUrArArArCrCrA | OOOOO OOOOO OOOOO O |
| | | | rUrCmCmUmG | |
| WV- | 79 | UCUCUUUCCAUGGAAG | mU*mC*mU*rC*rU*rU*rU*rC*rC*rA*rU* | XXXXX XXXXX XXXXX |
| 20704 | | GUUCUAAACCAUCCUG | rG*rG*rA*rA*rG*rGrUrUrCrUrArArArCrC | XOOOO OOOOO OOOOO O |
| | | | rArUrCmCmUmG | |
| WV- | 80 | UCUCUUUCCAUGGAAG | mUmCmUrCrUrUrUrCrCrArUrGrGrArArGrGrU | OOOOO OOOOO OOOOO |
| 20706 | | GUUCUAAACCAUCCUG | rUrCrUrArArArCrCrArUrCmC*mU*mG | OOOOO OOOOO OOOOX X |
| WV- | 81 | UCUCUUUCCAUGGAAG | mUmCmUrCrUrUrUrCrCrArUrGrGrArArGrGrU | OOOOO OOOOO OOOOO |
| 20707 | | GUUCUAAACCAUCCUG | rUrCrUrArArArCrCrArUrC*mC*mU*mG | OOOOO OOOOO OOOXX X |
| WV- | 82 | UCUCUUUCCAUGGAAG | mUmCmUrCrUrUrUrCrCrArUrGrGrArArGrGrU | OOOOO OOOOO OOOOO |
| 20708 | | GUUCUAAACCAUCCUG | rUrCrUrArArArCrCrArU*rC*mC*mU*mG | OOOOO OOOOO OOXXX X |
| WV- | 83 | UCUCUUUCCAUGGAAG | mUmCmUrCrUrUrUrCrCrArUrGrGrArArGrGrU | OOOOO OOOOO OOOOO |
| 20709 | | GUUCUAAACCAUCCUG | rUrCrUrArA*rArCrCrArU*rC*mC*mU*mG | OOOOO OOXOO OOXXX X |
| WV- | 84 | UCUCUUUCCAUGGAAG | mUmCmUrCrUrUrUrCrCrArUrGrGrArArGrGrU | OOOOO OOOOO OOOOO |
| 20710 | | GUUCUAAACCAUCCUG | rUrCrUrA*rA*rArCrCrArU*rC*mC*mU*mG | OOOOO OXXOO OOXXX X |
| WV- | 85 | UCUCUUUCCAUGGAAG | mUmCmUrCrUrUrUrCrCrArUrGrGrArArGrGrU | OOOOO OOOOO OOOOO |
| 20711 | | GUUCUAAACCAUCCUG | rUrCrU*rA*rA*rArCrCrArU*rC*mC*mU* | OOOOO XXXOO OOXXX X |
| | | | mG | |
| WV- | 86 | UCUCUUUCCAUGGAAG | mUmCmUrCrUrUrUrCrCrArUrGrGrArArGrGrU | OOOOO OOOOO OOOOO |
| 20712 | | GUUCUAAACCAUCCUG | rUrC*rU*rA*rA*rArCrCrArU*rC*mC*mU* | OOOOX XXXOO OOXXX X |
| | | | mG | |
| WV- | 87 | UCUCUUUCCAUGGAAG | mUmCmUrCrUrUrUrCrCrArUrGrGrArArGrGrU | OOOOO OOOOO OOOOO |
| 20713 | | GUUCUAAACCAUCCUG | rU*rC*rU*rA*rA*rArCrCrArU*rC*mC*mU | OOOXX XXXOO OOXXX X |
| | | | *mG | |
| WV- | 88 | UCUCUUUCCAUGGAAG | mUmCmUrCrUrUrUrCrCrArUrGrGrArArGrGrU | OOOOO OOOOO OOOOO |
| 20714 | | GUUCUAAACCAUCCUG | *rU*rC*rU*rA*rA*rArCrCrArU*rC*mC* | OOXXX XXXOO OOXXX X |
| | | | mU*mG | |
| WV- | 89 | UCUCUUUCCAUGGAAG | mUmCmUrCrUrUrUrCrCrArUrGrGrArArGrG* | OOOOO OOOOO OOOOO |
| 20715 | | GUUCUAAACCAUCCUG | rU*rU*rC*rU*rA*rA*rArCrCrArU*rC*mC* | OXXXX XXXOO OOXXX X |
| | | | mU*mG | |
| WV- | 90 | UCUCUUUCCAUGGAAG | mUmCmUrCrUrUrUrCrCrArUrGrGrArArG*rG | OOOOO OOOOO OOOOO |
| 20716 | | GUUCUAAACCAUCCUG | *rU*rU*rC*rU*rA*rA*rArCrCrArU*rC*mC | XXXXX XXXOO OOXXX X |
| | | | *mU*mG | |
| WV- | 91 | UCUCUUUCCAUGGAAG | mUmCmUrCrUrUrUrCrCrArUrGrGrArArGrGrU | OOOOO OOOOO OOOOO |
| 20717 | | GUUCUAAACCAUCCUG | rUrCrUrArArArCrCrA*rU*rC*mC*mU*mG | OOOOO OOOOO OXXXX X |
| WV- | 92 | UCUCUUUCCAUGGAAG | mUmCmUrCrUrUrUrCrCrArUrGrGrArArGrGrU | OOOOO OOOOO OOOOO |
| 20718 | | GUUCUAAACCAUCCUG | rUrCrUrArArA*rCrCrA*rU*rC*mC*mU*mG | OOOOO OOOXO OXXXX X |
| WV- | 93 | UCUCUUUCCAUGGAAG | mUmCmUrCrUrUrUrCrCrArUrGrGrArArGrGrU | OOOOO OOOOO OOOOO |
| 20719 | | GUUCUAAACCAUCCUG | rUrCrUrArA*rA*rCrCrA*rU*rC*mC*mU* | OOOOO OOXXO OXXXX X |
| | | | mG | |
| WV- | 94 | UCUCUUUCCAUGGAAG | mUmCmUrCrUrUrUrCrCrArUrGrGrArArGrGrU | OOOOO OOOOO OOOOO |
| 20720 | | GUUCUAAACCAUCCUG | rUrCrUrA*rA*rA*rCrCrA*rU*rC*mC*mU* | OOOOO OXXXO OXXXX X |
| | | | mG | |
| WV- | 95 | UCUCUUUCCAUGGAAG | mUmCmUrCrUrUrUrCrCrArUrGrGrArArGrGrU | OOOOO OOOOO OOOOO |
| 20721 | | GUUCUAAACCAUCCUG | rUrCrU*rA*rA*rA*rCrCrA*rU*rC*mC*mU | OOOOO XXXXO OXXXX X |
| | | | *mG | |
| WV- | 96 | UCUCUUUCCAUGGAAG | mUmCmUrCrUrUrUrCrCrArUrGrGrArArGrGrU | OOOOO OOOOO OOOOO |
| 20722 | | GUUCUAAACCAUCCUG | rUrC*rU*rA*rA*rA*rCrCrA*rU*rC*mC* | OOOOX XXXXO OXXXX X |
| | | | mU*mG | |
| WV- | 97 | UCUCUUUCCAUGGAAG | mUmCmUrCrUrUrUrCrCrArUrGrGrArArGrGrU | OOOOO OOOOO OOOOO |
| 20723 | | GUUCUAAACCAUCCUG | rU*rC*rU*rA*rA*rA*rCrCrA*rU*rC*mC* | OOOXX XXXXO OXXXXX |
| | | | mU*mG | |
| WV- | 98 | UCUCUUUCCAUGGAAG | mUmCmUrCrUrUrUrCrCrArUrGrGrArArGrGrU | OOOOO OOOOO OOOOO |
| 20724 | | GUUCUAAACCAUCCUG | *rU*rC*rU*rA*rA*rA*rCrCrA*rU*rC*mC* | OOXXX XXXXO OXXXXX |
| | | | mU*mG | |
| WV- | 99 | UCUCUUUCCAUGGAAG | mUmCmUrCrUrUrUrCrCrArUrGrGrArArGrG* | OOOOO OOOOO OOOOO |
| 20725 | | GUUCUAAACCAUCCUG | rU*rU*rC*rU*rA*rA*rA*rCrCrA*rU*rC* | OXXXX XXXXO OXXXXX |
| | | | mC*mU*mG | |
| WV- | 100 | UCUCUUUCCAUGGAAG | mUmCmUrCrUrUrUrCrCrArUrGrGrArArG*rG | OOOOO OOOOO OOOOO |
| 20726 | | GUUCUAAACCAUCCUG | *rU*rU*rC*rU*rA*rA*rA*rCrCrA*rU*rC* | XXXXX XXXXO OXXXX X |
| | | | mC*mU*mG | |
| WV- | 101 | UCUCUUUCCAUGGAAG | mUmCmUrCrUrUrUrCrCrArUrGrGrArArGrGrU | OOOOO OOOOO OOOOO |
| 20727 | | GUUCUAAACCAUCCUG | rUrCrUrArArA*rCrCrArUrCmCmUmG | OOOOO OOOXO OOOOO O |
| WV- | 102 | UCUCUUUCCAUGGAAG | mUmCmUrCrUrUrUrCrCrArUrGrGrArArGrGrU | OOOOO OOOOO OOOOO |
| 20728 | | GUUCUAAACCAUCCUG | rUrCrUrArArArC*rCrArUrCmCmUmG | OOOOO OOOOX OOOOO O |
| WV- | 103 | UCUCUUUCCAUGGAAG | mUmCmUrCrUrUrUrCrCrArUrGrGrArArGrGrU | OOOOO OOOOO OOOOO |
| 20729 | | GUUCUAAACCAUCCUG | rUrCrUrArArArCrC*rArUrCmCmUmG | OOOOO OOOOO XOOOO O |
| WV- | 104 | UCUCUUUCCAUGGAAG | mUmCmUrCrUrUrUrCrCrArUrGrGrArArGrGrU | OOOOO OOOOO OOOOO |
| 20730 | | GUUCUAAACCAUCCUG | rUrCrUrArArArCrCrA*rUrCmCmUmG | OOOOO OOOOO OXOOOO |
| WV- | 105 | UCUCUUUCCAUGGAAG | mUmCmUrCrUrUrUrCrCrArUrGrGrArArGrGrU | OOOOO OOOOO OOOOO |
| 20731 | | GUUCUAAACCAUCCUG | rUrCrUrArArA*rC*rC*rA*rUrCmCmUmG | OOOOO OOOXX XXOOOO |
| WV- | 106 | UCUCUUUCCAUGGAAG | mUmCmUrCrUrUrUrCrCrArUrGrGrArArGrGrU | OOOOO OOOOO OOOOO |
| 20732 | | GUUCUAAACCAUCCUG | rUrCrUrArArArC*rC*rArUrCmCmUmG | OOOOO OOOOX XOOOO O |
| WV- | 107 | UCUCUUUCCAUGGAAG | mU*mC*mU*rC*rU*rU*rU*rC*rC*rA*rU* | XXXXX XXXXX XXXXX |
| 20733 | | GUUCUAAACCAUCCUG | rG*rG*rA*rA*rG*rG*rU*rU*rC*rU*rA*rA | XXXXX XXXXX XXXXX X |
| | | | *rA*rC*rC*rA*rU*rC*mC*mU*mG | |
| WV- | 108 | UCUCUUUCCAUGGAAG | mUmCmUmCmUrUrUrCrCrArUrGrGrArArGrG | OOOOO OOOOO OOOOO |
| 20734 | | GUUCUAAACCAUCCUG | rUrUrCrUrArArArCrCrArUrCmCmUmG | OOOOO OOOOO OOOOO O |
| WV- | 109 | UCUCUUUCCAUGGAAG | mUmCmUmCmUmUmUrCrCrArUrGrGrArArGrG | OOOOO OOOOO OOOOO |
| 20735 | | GUUCUAAACCAUCCUG | rUrUrCrUrArArArCrCrArUrCmCmUmG | OOOOO OOOOO OOOOO O |
| WV- | 110 | UCUCUUUCCAUGGAAG | mUmCmUmCmUmUmUmCmCrArUrGrGrArArG | OOOOO OOOOO OOOOO |
| 20736 | | GUUCUAAACCAUCCUG | rGrUrUrCrUrArArArCrCrArUrCmCmUmG | OOOOO OOOOO OOOOO O |
| WV- | 111 | UCUCUUUCCAUGGAAG | mUmCmUmCmUmUmUmCmCmAmUrGrGrArArG | OOOOO OOOOO OOOOO |
| 20737 | | GUUCUAAACCAUCCUG | rGrUrUrCrUrArArArCrCrArUrCmCmUmG | OOOOO OOOOO OOOOO O |
| WV- | 112 | UCUCUUUCCAUGGAAG | mUmCmUmCmUmUmUmCmCmAmUmGmGrArArG | OOOOO OOOOO OOOOO |
| 20738 | | GUUCUAAACCAUCCUG | rGrUrUrCrUrArArArCrCrArUrCmCmUmG | OOOOO OOOOO OOOOO O |
| WV- | 113 | UCUCUUUCCAUGGAAG | mUmCmUmCmUmUmUmCmCmAmUmGmGmAmA | OOOOO OOOOO OOOOO |
| 20739 | | GUUCUAAACCAUCCUG | rGrGrUrUrCrUrArArArCrCrArUrCmCmUmG | OOOOO OOOOO OOOOO O |
| WV- | 114 | UCUCUUUCCAUGGAAG | mUmCmUmCmUmUmUmCmCmAmUmG | OOOOO OOOOO OOOOO |
| 20740 | | GUUCUAAACCAUCCUG | mGmAmAmGmGrUrUrCrUrArArArCrCrArU | OOOOO OOOOO OOOOO O |
| | | | rCmCmUmG | |
| WV- | 115 | UCUCUUUCCAUGGAAG | mUmCmUrCrUrUrUrCrCrArUrGrGrArArGrGrU | OOOOO OOOOO OOOOO |
| 20741 | | GUUCUAAACCAUCCUG | rUrCrUrArArArCrCrAmUmCmCmUmG | OOOOO OOOOO OOOOO O |
| WV- | 116 | UCUCUUUCCAUGGAAG | mUmCmUrCrUrUrUrCrCrArUrGrGrArArGrGrU | OOOOO OOOOO OOOOO |
| 20742 | | GUUCUAAACCAUCCUG | rUrCrUrArAmArCrCrAmUmCmCmUmG | OOOOO OOOOO OOOOO O |
| WV- | 117 | UCUCUUUCCAUGGAAG | mUmCmUrCrUrUrUrCrCrArUrGrGrArArGrGrU | OOOOO OOOOO OOOOO |
| 20743 | | GUUCUAAACCAUCCUG | rUrCrUmAmAmArCrCrAmUmCmCmUmG | OOOOO OOOOO OOOOO O |
| WV- | 118 | UCUCUUUCCAUGGAAG | mUmCmUrCrUrUrUrCrCrArUrGrGrArArGrGrU | OOOOO OOOOO OOOOO |
| 20744 | | GUUCUAAACCAUCCUG | rUmCmUmAmAmArCrCrAmUmCmCmUmG | OOOOO OOOOO OOOOO O |
| WV- | 119 | UCUCUUUCCAUGGAAG | mUmCmUrCrUrUrUrCrCrArUrGrGrArArG | OOOOO OOOOO OOOOO |
| 20745 | | GUUCUAAACCAUCCUG | rGmUmUmCmUmAmAmArCrCrAmUmCmCmUmG | OOOOO OOOOO OOOOO O |
| WV- | 120 | UCUCUUUCCAUGGAAG | mUmCmUrCrUrUrUrCrCrArUrGrGrA | OOOOO OOOOO OOOOO |
| 20746 | | GUUCUAAACCAUCCUG | rAmGmGmUmUmCmUmAmAmArCrC | OOOOO OOOOO OOOOO O |
| | | | rAmUmCmCmUmG | |
| WV- | 121 | UCUCUUUCCAUGGAAG | fUfCfUrCrUrUrUrCrCrArUrGrGrArArGrGrUrU | OOOOO OOOOO OOOOO |
| 20747 | | GUUCUAAACCAUCCUG | rCrUrArArArCrCrArUrCfCfUfG | OOOOO OOOOO OOOOO O |
| WV- | 122 | UCUCUUUCCAUGGAAG | fUfCfUfCfUrUrUrCrCrArUrGrGrArArGrGrUrU | OOOOO OOOOO OOOOO |
| 20748 | | GUUCUAAACCAUCCUG | rCrUrArArArCrCrArUrCfCfUfG | OOOOO OOOOO OOOOO O |
| WV- | 123 | UCUCUUUCCAUGGAAG | fUfCfUfCfUfUfUrCrCrArUrGrGrArArGrGrUrU | OOOOO OOOOO OOOOO |
| 20749 | | GUUCUAAACCAUCCUG | rCrUrArArArCrCrArUrCfCfUfG | OOOOO OOOOO OOOOO O |
| WV- | 124 | UCUCUUUCCAUGGAAG | fUfCfUfCfUfUfUfCfCrArUrGrGrArArGrGrUrUrC | OOOOO OOOOO OOOOO |
| 20750 | | GUUCUAAACCAUCCUG | rUrArArArCrCrArUrCfCfUfG | OOOOO OOOOO OOOOO O |
| WV- | 125 | UCUCUUUCCAUGGAAG | fUfCfUfCfUfUfUfCfCfAfUrGrGrArArGrGrUrUrC | OOOOO OOOOO OOOOO |
| 20751 | | GUUCUAAACCAUCCUG | rUrArArArCrCrArUrCfCfUfG | OOOOO OOOOO OOOOO O |
| WV- | 126 | UCUCUUUCCAUGGAAG | fUfCfUfCfUfUfUfCfCfAfUfGfGrArArGrGrUrUrCrU | OOOOO OOOOO OOOOO |
| 20752 | | GUUCUAAACCAUCCUG | rArArArCrCrArUrCfCfUfG | OOOOO OOOOO OOOOO O |
| WV- | 127 | UCUCUUUCCAUGGAAG | fUfCfUfCfUfUfUfCfCfAfUfGfGfAfArGrGrUrUrCrU | OOOOO OOOOO OOOOO |
| 20753 | | GUUCUAAACCAUCCUG | rArArArCrCrArUrCfCfUfG | OOOOO OOOOO OOOOO O |
| WV- | 128 | UCUCUUUCCAUGGAAG | fUfCfUfCfUfUfUfCfCfAfUfGfGfAfAfGfGrUrUrCrU | OOOOO OOOOO OOOOO |
| 20754 | | GUUCUAAACCAUCCUG | rArArArCrCrArUrCfCfUfG | OOOOO OOOOO OOOOO O |
| WV- | 129 | UCUCUUUCCAUGGAAG | fUfCfUrCrUrUrUrCrCrArUrGrGrArArGrGrUrU | OOOOO OOOOO OOOOO |
| 20755 | | GUUCUAAACCAUCCUG | rCrUrArArArCrCrAfUfCfCfUfG | OOOOO OOOOO OOOOO O |
| WV- | 130 | UCUCUUUCCAUGGAAG | fUfCfUrCrUrUrUrCrCrArUrGrGrArArGrGrUrU | OOOOO OOOOO OOOOO |
| 20756 | | GUUCUAAACCAUCCUG | rCrUrArAfArCrCrAfUfCfCfUfG | OOOOO OOOOO OOOOO O |
| WV- | 131 | UCUCUUUCCAUGGAAG | fUfCfUrCrUrUrUrCrCrArUrGrGrArArGrGrUrU | OOOOO OOOOO OOOOO |
| 20757 | | GUUCUAAACCAUCCUG | rCrUfAfAfArCrCrAfUfCfCfUfG | OOOOO OOOOO OOOOO O |
| WV- | 132 | UCUCUUUCCAUGGAAG | fUfCfUrCrUrUrUrCrCrArUrGrGrArArGrGrU | OOOOO OOOOO OOOOO |
| 20758 | | GUUCUAAACCAUCCUG | rUfCfUfAfAfArCrCrAfUfCfCfUfG | OOOOO OOOOO OOOOO O |
| WV- | 133 | UCUCUUUCCAUGGAAG | fUfCfUrCrUrUrUrCrCrArUrGrGrArArG | OOOOO OOOOO OOOOO |
| 20759 | | GUUCUAAACCAUCCUG | rGfUfUfCfUfAfAfArCrCrAfUfCfCfUfG | OOOOO OOOOO OOOOO O |
| WV- | 134 | UCUCUUUCCAUGGAAG | fUfCfUrCrUrUrUrCrCrArUrGrGrA | OOOOO OOOOO OOOOO |
| 20760 | | GUUCUAAACCAUCCUG | rAfGfGfUfUfCfUfAfAfArCrCrAfUfCfCfUfG | OOOOO OOOOO OOOOO O |
| WV- | 135 | UCUCUUUCCAUGGAAG | mU*mC*mU*rC*rU*rU*rU*rC*rC*rA*rU* | XXXXX XXXXX XXXXX |
| 22259 | | GUUCUAAACCAUCCU | rG*rG*rA*rA*rG*rG*rU*rU*rC*rU*rA*rA | XXXXX XXXXX XXXXX |
| | | | *rA*rC*rC*rA*rU*mC*mC*mU | |
| WV- | 136 | UCUCUUUCCAUGGAAG | mU*mC*mU*rC*rU*rU*rU*rC*rC*rA*rU* | XXXXX XXXXX XXXXX |
| 22260 | | GUUCUAAACCAUCC | rG*rG*rA*rA*rG*rG*rU*rU*rC*rU*rA*rA | XXXXX XXXXX XXXX |
| | | | *rA*rC*rC*rA*mU*mC*mC | |
| WV- | 137 | UCUCUUUCCAUGGAAG | mU*mC*mU*rC*rU*rU*rU*rC*rC*rA*rU* | XXXXX XXXXX XXXXX |
| 22261 | | GUUCUAAACCAUC | rG*rG*rA*rA*rG*rG*rU*rU*rC*rU*rA*rA | XXXXX XXXXX XXX |
| | | | *rA*rC*rC*rA*mU*mC | |
| WV- | 138 | CUCUUUCCAUGGAAGG | mC*mU*mC*rU*rU*rU*rC*rC*rA*rU*rG* | XXXXX XXXXX XXXXX |
| 22262 | | UUCUAAACCAUCCUG | rG*rA*rA*rG*rG*rU*rU*rC*rU*rA*rA*rA | XXXXX XXXXX XXXXX |
| | | | *rC*rC*rA*rU*rC*mC*mU*mG | |
| WV- | 139 | CUCUUUCCAUGGAAGG | mC*mU*mC*rU*rU*rU*rC*rC*rA*rU*rG* | XXXXX XXXXX XXXXX |
| 22263 | | UUCUAAACCAUCCU | rG*rA*rA*rG*rG*rU*rU*rC*rU*rA*rA*rA | XXXXX XXXXX XXXX |
| | | | *rC*rC*rA*rU*mC*mC*mU | |
| WV- | 140 | CUCUUUCCAUGGAAGG | mC*mU*mC*rU*rU*rU*rC*rC*rA*rU*rG* | XXXXX XXXXX XXXXX |
| 22264 | | UUCUAAACCAUCC | rG*rA*rA*rG*rG*rU*rU*rC*rU*rA*rA*rA | XXXXX XXXXX XXX |
| | | | *rC*rC*rA*mU*mC*mC | |
| WV- | 141 | CUCUUUCCAUGGAAGG | mC*mU*mC*rU*rU*rU*rC*rC*rA*rU*rG* | XXXXX XXXXX XXXXX |
| 22265 | | UUCUAAACCAUC | rG*rA*rA*rG*rG*rU*rU*rC*rU*rA*rA*rA | XXXXX XXXXX XX |
| | | | *rC*rC*rA*mU*mC | |
| WV- | 142 | UCUUUCCAUGGAAGGU | mU*mC*mU*rU*rU*rC*rC*rA*rU*rG*rG* | XXXXX XXXXX XXXXX |
| 22266 | | UCUAAACCAUCCUG | rA*rA*rG*rG*rU*rU*rC*rU*rA*rA*rA*rC | XXXXX XXXXX XXXX |
| | | | *rC*rA*rU*rC*mC*mU*mG | |
| WV- | 143 | UCUUUCCAUGGAAGGU | mU*mC*mU*rU*rU*rC*rC*rA*rU*rG*rG* | XXXXX XXXXX XXXXX |
| 22267 | | UCUAAACCAUCCU | rA*rA*rG*rG*rU*rU*rC*rU*rA*rA*rA*rC | XXXXX XXXXX XXX |
| | | | *rC*rA*rU*mC*mC*mU | |
| WV- | 144 | UCUUUCCAUGGAAGGU | mU*mC*mU*rU*rU*rC*rC*rA*rU*rG*rG* | XXXXX XXXXX XXXXX |
| 22268 | | UCUAAACCAUCC | rA*rA*rG*rG*rU*rU*rC*rU*rA*rA*rA*rC | XXXXX XXXXX XX |
| | | | *rC*rA*mU*mC*mC | |
| WV- | 145 | UCUUUCCAUGGAAGGU | mU*mC*mU*rU*rU*rC*rC*rA*rU*rG*rG* | XXXXX XXXXX XXXXX |
| 22269 | | UCUAAACCAUC | rA*rA*rG*rG*rU*rU*rC*rU*rA*rA*rA*rC | XXXXX XXXXX X |
| | | | *rC*rA*mU*mC | |
| WV- | 146 | CUUUCCAUGGAAGGUU | mC*mU*mU*rU*rC*rC*rA*rU*rG*rG*rA* | XXXXX XXXXX XXXXX |
| 22270 | | CUAAACCAUCCUG | rA*rG*rG*rU*rU*rC*rU*rA*rA*rA*rC*rC | XXXXX XXXXX XXX |
| | | | *rA*rU*rC*mC*mU*mG | |
| WV- | 147 | CUUUCCAUGGAAGGUU | mC*mU*mU*rU*rC*rC*rA*rU*rG*rG*rA* | XXXXX XXXXX XXXXX |
| 22271 | | CUAAACCAUCCU | rA*rG*rG*rU*rU*rC*rU*rA*rA*rA*rC*rC | XXXXX XXXXX XX |
| | | | *rA*rU*mC*mC*mU | |
| WV- | 148 | CUUUCCAUGGAAGGUU | mC*mU*mU*rU*rC*rC*rA*rU*rG*rG*rA* | XXXXX XXXXX XXXXX |
| 22272 | | CUAAACCAUCC | rA*rG*rG*rU*rU*rC*rU*rA*rA*rA*rC*rC | XXXXX XXXXX X |
| | | | *rA*mU*mC*mC | |
| WV- | 149 | CUUUCCAUGGAAGGUU | mC*mU*mU*rU*rC*rC*rA*rU*rG*rG*rA* | XXXXX XXXXX XXXXX |
| 22273 | | CUAAACCAUC | rA*rG*rG*rU*rU*rC*rU*rA*rA*rA*rC*rC | XXXXX XXXXX |
| | | | *rA*mU*mC | |
| WV- | 150 | UCUCUUUCCAUGGAAG | mU*mC*mU*mC*mU*mU*mU*mC*mC*mA | XXXXX XXXXX XXXXX |
| 22290 | | GUUCUAAACCAUCCUG | *mU*mG*mG*mA*mA*mG*mG*mU*mU* | XXXXX XXXXX XXXXX X |
| | | | mC*mU*mA*mA*mA*rC*rC*rA*mU*mC* | |
| | | | mC*mU*mG | |
| WV- | 151 | UCUCUUUCCAUGGAAG | mU*mC*mU*mC*mU*mU*mU*mC*mC*mA | XXXXX XXXXX XXXXX |
| 22291 | | GUUCUAAACCAUCCU | *mU*mG*mG*mA*mA*mG*mG*mU*mU* | XXXXX XXXXX XXXXX |
| | | | mC*mU*mA*mA*mA*rC*rC*rA*mU*mC* | |
| | | | mC*mU | |
| WV- | 152 | UCUCUUUCCAUGGAAG | mU*mC*mU*mC*mU*mU*mU*mC*mC*mA | XXXXX XXXXX XXXXX |
| 22292 | | GUUCUAAACCAUCC | *mU*mG*mG*mA*mA*mG*mG*mU*mU* | XXXXX XXXXX XXXX |
| | | | mC*mU*mA*mA*mA*rC*rC*rA*mU*mC* | |
| | | | mC | |
| WV- | 153 | UCUCUUUCCAUGGAAG | mU*mC*mU*mC*mU*mU*mU*mC*mC*mA | XXXXX XXXXX XXXXX |
| 22293 | | GUUCUAAACCAUC | *mU*mG*mG*mA*mA*mG*mG*mU*mU* | XXXXX XXXXX XXX |
| | | | mC*mU*mA*mA*mA*rC*rC*rA*mU*mC | |
| WV- | 154 | CUCUUUCCAUGGAAGG | mC*mU*mC*mU*mU*mU*mC*mC*mA*mU | XXXXX XXXXX XXXXX |
| 22294 | | UUCUAAACCAUCCUG | *mG*mG*mA*mA*mG*mG*mU*mU*mC* | XXXXX XXXXX XXXXX |
| | | | mU*mA*mA*mA*rC*rC*rA*mU*mC*mC* | |
| | | | mU*mG | |
| WV- | 155 | CUCUUUCCAUGGAAGG | mC*mU*mC*mU*mU*mU*mC*mC*mA*mU | XXXXX XXXXX XXXXX |
| 22295 | | UUCUAAACCAUCCU | *mG*mG*mA*mA*mG*mG*mU*mU*mC* | XXXXX XXXXX XXXX |
| | | | mU*mA*mA*mA*rC*rC*rA*mU*mC*mC* | |
| | | | mU | |
| WV- | 156 | CUCUUUCCAUGGAAGG | mC*mU*mC*mU*mU*mU*mC*mC*mA*mU | XXXXX XXXXX XXXXX |
| 22296 | | UUCUAAACCAUCC | *mG*mG*mA*mA*mG*mG*mU*mU*mC* | XXXXX XXXXX XXX |
| | | | mU*mA*mA*mA*rC*rC*rA*mU*mC*mC | |
| WV- | 157 | CUCUUUCCAUGGAAGG | mC*mU*mC*mU*mU*mU*mC*mC*mA*mU | XXXXX XXXXX XXXXX |
| 22297 | | UUCUAAACCAUC | *mG*mG*mA*mA*mG*mG*mU*mU*mC* | XXXXX XXXXX XX |
| | | | mU*mA*mA*mA*rC*rC*rA*mU*mC | |
| WV- | 158 | UCUUUCCAUGGAAGGU | mU*mC*mU*mU*mU*mC*mC*mA*mU*mG | XXXXX XXXXX XXXXX |
| 22298 | | UCUAAACCAUCCUG | *mG*mA*mA*mG*mG*mU*mU*mC*mU* | XXXXX XXXXX XXXX |
| | | | mA*mA*mA*rC*rC*rA*mU*mC*mC*mU* | |
| | | | mG | |
| WV- | 159 | UCUUUCCAUGGAAGGU | mU*mC*mU*mU*mU*mC*mC*mA*mU*mG | XXXXX XXXXX XXXXX |
| 22299 | | UCUAAACCAUCCU | *mG*mA*mA*mG*mG*mU*mU*mC*mU* | XXXXX XXXXX XXX |
| | | | mA*mA*mA*rC*rC*rA*mU*mC*mC*mU | |
| WV- | 160 | UCUUUCCAUGGAAGGU | mU*mC*mU*mU*mU*mC*mC*mA*mU*mG | XXXXX XXXXX XXXXX |
| 22300 | | UCUAAACCAUCC | *mG*mA*mA*mG*mG*mU*mU*mC*mU* | XXXXX XXXXX XX |
| | | | mA*mA*mA*rC*rC*rA*mU*mC*mC | |
| WV- | 161 | UCUUUCCAUGGAAGGU | mU*mC*mU*mU*mU*mC*mC*mA*mU*mG | XXXXX XXXXX XXXXX |
| 22301 | | UCUAAACCAUC | *mG*mA*mA*mG*mG*mU*mU*mC*mU* | XXXXX XXXXX X |
| | | | mA*mA*mA*rC*rC*rA*mU*mC | |
| WV- | 162 | CUUUCCAUGGAAGGUU | mC*mU*mU*mU*mC*mC*mA*mU*mG*mG | XXXXX XXXXX XXXXX |
| 22302 | | CUAAACCAUCCUG | *mA*mA*mG*mG*mU*mU*mC*mU*mA* | XXXXX XXXXX XXX |
| | | | mA*mA*rC*rC*rA*mU*mC*mC*mU*mG | |
| WV- | 163 | CUUUCCAUGGAAGGUU | mC*mU*mU*mU*mC*mC*mA*mU*mG*mG | XXXXX XXXXX XXXXX |
| 22303 | | CUAAACCAUCCU | *mA*mA*mG*mG*mU*mU*mC*mU*mA* | XXXXX XXXXX XX |
| | | | mA*mA*rC*rC*rA*mU*mC*mC*mU | |
| WV- | 164 | CUUUCCAUGGAAGGUU | mC*mU*mU*mU*mC*mC*mA*mU*mG*mG | XXXXX XXXXX XXXXX |
| 22304 | | CUAAACCAUCC | *mA*mA*mG*mG*mU*mU*mC*mU*mA* | XXXXX XXXXX X |
| | | | mA*mA*rC*rC*rA*mU*mC*mC | |
| WV- | 165 | CUUUCCAUGGAAGGUU | mC*mU*mU*mU*mC*mC*mA*mU*mG*mG | XXXXX XXXXX XXXXX |
| 22305 | | CUAAACCAUC | *mA*mA*mG*mG*mU*mU*mC*mU*mA* | XXXXX XXXXX |
| | | | mA*mA*rC*rC*rA*mU*mC | |
| WV- | 166 | UCUCUUUCCAUGGAAG | fU*fC*fU*fC*fU*fU*fU*fC*fC*fA*fU*fG | XXXXX XXXXX XXXXX |
| 22306 | | GUUCUAAACCAUCCUG | *fG*fA*fA*fG*mG*mU*mU*mC*mU*mA* | XXXXX XXXXX XXXXX X |
| | | | mA*mA*rC*rC*rA*mU*mC*mC*mU*mG | |
| WV- | 167 | UCUCUUUCCAUGGAAG | fU*fC*fU*fC*fU*fU*fU*fC*fC*fA*fU*fG | XXXXX XXXXX XXXXX |
| 22307 | | GUUCUAAACCAUCCU | *fG*fA*fA*fG*mG*mU*mU*mC*mU*mA* | XXXXX XXXXX XXXXX |
| | | | mA*mA*rC*rC*rA*mU*mC*mC*mU | |
| WV- | 168 | UCUCUUUCCAUGGAAG | fU*fC*fU*fC*fU*fU*fU*fC*fC*fA*fU*fG | XXXXX XXXXX XXXXX |
| 22308 | | GUUCUAAACCAUCC | *fG*fA*fA*fG*mG*mU*mU*mC*mU*mA* | XXXXX XXXXX XXXX |
| | | | mA*mA*rC*rC*rA*mU*mC*mC | |
| WV- | 169 | UCUCUUUCCAUGGAAG | fU*fC*fU*fC*fU*fU*fU*fC*fC*fA*fU*fG | XXXXX XXXXX XXXXX |
| 22309 | | GUUCUAAACCAUC | *fG*fA*fA*fG*mG*mU*mU*mC*mU*mA* | XXXXX XXXXX XXX |
| | | | mA*mA*rC*rC*rA*mU*mC | |
| WV- | 170 | CUCUUUCCAUGGAAGG | fC*fU*fC*fU*fU*fU*fC*fC*fA*fU*fG*fG | XXXXX XXXXX XXXXX |
| 22310 | | UUCUAAACCAUCCUG | *fA*fA*fG*mG*mU*mU*mC*mU*mA*mA | XXXXX XXXXX XXXXX |
| | | | *mA*rC*rC*rA*mU*mC*mC*mU*mG | |
| WV- | 171 | CUCUUUCCAUGGAAGG | fC*fU*fC*fU*fU*fU*fC*fC*fA*fU*fG*fG | XXXXX XXXXX XXXXX |
| 22311 | | UUCUAAACCAUCCU | *fA*fA*fG*mG*mU*mU*mC*mU*mA*mA | XXXXX XXXXX XXXX |
| | | | *mA*rC*rC*rA*mU*mC*mC*mU | |
| WV- | 172 | CUCUUUCCAUGGAAGG | fC*fU*fC*fU*fU*fU*fC*fC*fA*fU*fG*fG | XXXXX XXXXX XXXXX |
| 22312 | | UUCUAAACCAUCC | *fA*fA*fG*mG*mU*mU*mC*mU*mA*mA | XXXXX XXXXX XXX |
| | | | *mA*rC*rC*rA*mU*mC*mC | |
| WV- | 173 | CUCUUUCCAUGGAAGG | fC*fU*fC*fU*fU*fU*fC*fC*fA*fU*fG*fG | XXXXX XXXXX XXXXX |
| 22313 | | UUCUAAACCAUC | *fA*fA*fG*mG*mU*mU*mC*mU*mA*mA | XXXXX XXXXX XX |
| | | | *mA*rC*rC*rA*mU*mC | |
| WV- | 174 | UCUUUCCAUGGAAGGU | fU*fC*fU*fU*fU*fC*fC*fA*fU*fG*fG*fA | XXXXX XXXXX XXXXX |
| 22314 | | UCUAAACCAUCCUG | *fA*fG*mG*mU*mU*mC*mU*mA*mA*mA | XXXXX XXXXX XXXX |
| | | | *rC*rC*rA*mU*mC*mC*mU*mG | |
| WV- | 175 | UCUUUCCAUGGAAGGU | fU*fC*fU*fU*fU*fC*fC*fA*fU*fG*fG*fA | XXXXX XXXXX XXXXX |
| 22315 | | UCUAAACCAUCCU | *fA*fG*mG*mU*mU*mC*mU*mA*mA*mA | XXXXX XXXXX XXX |
| | | | *rC*rC*rA*mU*mC*mC*mU | |
| WV- | 176 | UCUUUCCAUGGAAGGU | fU*fC*fU*fU*fU*fC*fC*fA*fU*fG*fG*fA | XXXXX XXXXX XXXXX |
| 22316 | | UCUAAACCAUCC | *fA*fG*mG*mU*mU*mC*mU*mA*mA*mA | XXXXX XXXXX XX |
| | | | *rC*rC*rA*mU*mC*mC | |
| WV- | 177 | UCUUUCCAUGGAAGGU | fU*fC*fU*fU*fU*fC*fC*fA*fU*fG*fG*fA | XXXXX XXXXX XXXXX |
| 22317 | | UCUAAACCAUC | *fA*fG*mG*mU*mU*mC*mU*mA*mA*mA | XXXXX XXXXX X |
| | | | *rC*rC*rA*mU*mC | |
| WV- | 178 | CUUUCCAUGGAAGGUU | fC*fU*fU*fU*fC*fC*fA*fU*fG*fG*fA*fA | XXXXX XXXXX XXXXX |
| 22318 | | CUAAACCAUCCUG | *fG*mG*mU*mU*mC*mU*mA*mA*mA*rC | XXXXX XXXXX XXX |
| | | | *rC*rA*mU*mC*mC*mU*mG | |
| WV- | 179 | CUUUCCAUGGAAGGUU | fC*fU*fU*fU*fC*fC*fA*fU*fG*fG*fA*fA | XXXXX XXXXX XXXXX |
| 22319 | | CUAAACCAUCCU | *fG*mG*mU*mU*mC*mU*mA*mA*mA*rC | XXXXX XXXXX XX |
| | | | *rC*rA*mU*mC*mC*mU | |
| WV- | 180 | CUUUCCAUGGAAGGUU | fC*fU*fU*fU*fC*fC*fA*fU*fG*fG*fA*fA | XXXXX XXXXX XXXXX |
| 22320 | | CUAAACCAUCC | *fG*mG*mU*mU*mC*mU*mA*mA*mA*rC | XXXXX XXXXX X |
| | | | *rC*rA*mU*mC*mC | |
| WV- | 181 | CUUUCCAUGGAAGGUU | fC*fU*fU*fU*fC*fC*fA*fU*fG*fG*fA*fA | XXXXX XXXXX XXXXX |
| 22321 | | CUAAACCAUC | *fG*mG*mU*mU*mC*mU*mA*mA*mA*rC | XXXXX XXXXX |
| | | | *rC*rA*mU*mC | |
| WV- | 182 | UCUCUUUCCAUGGAAG | fU*fC*fU*fC*fU*fU*fU*fC*fC*fA*fU*fG | XXXXX XXXXX XXXXX |
| 22322 | | GUUCUAAACCAUCCUG | *fG*fA*fA*mG*mG*mU*mU*mC*mU*mA | XXXXX XXXXX XXXXX X |
| | | | *mA*mA*rC*rC*rA*mU*mC*mC*mU*mG | |
| WV- | 183 | UCUCUUUCCAUGGAAG | fU*fC*fU*fC*fU*fU*fU*fC*fC*fA*fU*fG | XXXXX XXXXX XXXXX |
| 22323 | | GUUCUAAACCAUCCU | *fG*fA*fA*mG*mG*mU*mU*mC*mU*mA | XXXXX XXXXX XXXXX |
| | | | *mA*mA*rC*rC*rA*mU*mC*mC*mU | |
| WV- | 184 | UCUCUUUCCAUGGAAG | fU*fC*fU*fC*fU*fU*fU*fC*fC*fA*fU*fG | XXXXX XXXXX XXXXX |
| 22324 | | GUUCUAAACCAUCC | *fG*fA*fA*mG*mG*mU*mU*mC*mU*mA | XXXXX XXXXX XXXX |
| | | | *mA*mA*rC*rC*rA*mU*mC*mC | |
| WV- | 185 | UCUCUUUCCAUGGAAG | fU*fC*fU*fC*fU*fU*fU*fC*fC*fA*fU*fG | XXXXX XXXXX XXXXX |
| 22325 | | GUUCUAAACCAUC | *fG*fA*fA*mG*mG*mU*mU*mC*mU*mA | XXXXX XXXXX XXX |
| | | | *mA*mA*rC*rC*rA*mU*mC | |
| WV- | 186 | CUCUUUCCAUGGAAGG | fC*fU*fC*fU*fU*fU*fC*fC*fA*fU*fG*fG | XXXXX XXXXX XXXXX |
| 22326 | | UUCUAAACCAUCCUG | *fA*fA*mG*mG*mU*mU*mC*mU*mA*mA | XXXXX XXXXX XXXXX |
| | | | *mA*rC*rC*rA*mU*mC*mC*mU*mG | |
| WV- | 187 | CUCUUUCCAUGGAAGG | fC*fU*fC*fU*fU*fU*fC*fC*fA*fU*fG*fG | XXXXX XXXXX XXXXX |
| 22327 | | UUCUAAACCAUCCU | *fA*fA*mG*mG*mU*mU*mC*mU*mA*mA | XXXXX XXXXX XXXX |
| | | | *mA*rC*rC*rA*mU*mC*mC*mU | |
| WV- | 188 | CUCUUUCCAUGGAAGG | fC*fU*fC*fU*fU*fU*fC*fC*fA*fU*fG*fG | XXXXX XXXXX XXXXX |
| 22328 | | UUCUAAACCAUCC | *fA*fA*mG*mG*mU*mU*mC*mU*mA*mA | XXXXX XXXXX XXX |
| | | | *mA*rC*rC*rA*mU*mC*mC | |
| WV- | 189 | CUCUUUCCAUGGAAGG | fC*fU*fC*fU*fU*fU*fC*fC*fA*fU*fG*fG | XXXXX XXXXX XXXXX |
| 22329 | | UUCUAAACCAUC | *fA*fA*mG*mG*mU*mU*mC*mU*mA*mA | XXXXX XXXXX XX |
| | | | *mA*rC*rC*rA*mU*mC | |
| WV- | 190 | UCUUUCCAUGGAAGGU | fU*fC*fU*fU*fU*fC*fC*fA*fU*fG*fG*fA | XXXXX XXXXX XXXXX |
| 22330 | | UCUAAACCAUCCUG | *fA*mG*mG*mU*mU*mC*mU*mA*mA* | XXXXX XXXXX XXXX |
| | | | mA*rC*rC*rA*mU*mC*mC*mU*mG | |
| WV- | 191 | UCUUUCCAUGGAAGGU | fU*fC*fU*fU*fU*fC*fC*fA*fU*fG*fG*fA | XXXXX XXXXX XXXXX |
| 22331 | | UCUAAACCAUCCU | *fA*mG*mG*mU*mU*mC*mU*mA*mA* | XXXXX XXXXX XXX |
| | | | mA*rC*rC*rA*mU*mC*mC*mU | |
| WV- | 192 | UCUUUCCAUGGAAGGU | fU*fC*fU*fU*fU*fC*fC*fA*fU*fG*fG*fA | XXXXX XXXXX XXXXX |
| 22332 | | UCUAAACCAUCC | *fA*mG*mG*mU*mU*mC*mU*mA*mA* | XXXXX XXXXX XX |
| | | | mA*rC*rC*rA*mU*mC*mC | |
| WV- | 193 | UCUUUCCAUGGAAGGU | fU*fC*fU*fU*fU*fC*fC*fA*fU*fG*fG*fA | XXXXX XXXXX XXXXX |
| 22333 | | UCUAAACCAUC | *fA*mG*mG*mU*mU*mC*mU*mA*mA* | XXXXX XXXXX X |
| | | | mA*rC*rC*rA*mU*mC | |
| WV- | 194 | CUUUCCAUGGAAGGUU | fC*fU*fU*fU*fC*fC*fA*fU*fG*fG*fA*fA | XXXXX XXXXX XXXXX |
| 22334 | | CUAAACCAUCCUG | *mG*mG*mU*mU*mC*mU*mA*mA*mA* | XXXXX XXXXX XXX |
| | | | rC*rC*rA*mU*mC*mC*mU*mG | |
| WV- | 195 | CUUUCCAUGGAAGGUU | fC*fU*fU*fU*fC*fC*fA*fU*fG*fG*fA*fA | XXXXX XXXXX XXXXX |
| 22335 | | CUAAACCAUCCU | *mG*mG*mU*mU*mC*mU*mA*mA*mA* | XXXXX XXXXX XX |
| | | | rC*rC*rA*mU*mC*mC*mU | |
| WV- | 196 | CUUUCCAUGGAAGGUU | fC*fU*fU*fU*fC*fC*fA*fU*fG*fG*fA*fA | XXXXX XXXXX XXXXX |
| 22336 | | CUAAACCAUCC | *mG*mG*mU*mU*mC*mU*mA*mA*mA* | XXXXX XXXXX X |
| | | | rC*rC*rA*mU*mC*mC | |
| WV- | 197 | CUUUCCAUGGAAGGUU | fC*fU*fU*fU*fC*fC*fA*fU*fG*fG*fA*fA | XXXXX XXXXX XXXXX |
| 22337 | | CUAAACCAUC | *mG*mG*mU*mU*mC*mU*mA*mA*mA* | XXXXX XXXXX |
| | | | rC*rC*rA*mU*mC | |
| WV- | 198 | UCUCUUUCCAUGGAAG | fU*fC*fU*fC*fU*fU*fU*fC*fC*fA*fU*fG | XXXXX XXXXX XXXXX |
| 22339 | | GUUCUAAACCAUCCU | *fG*fA*fA*fG*fG*mU*mU*mC*mU*mA* | XXXXX XXXXX XXXXX |
| | | | mA*mA*rC*rC*rA*mU*mC*mC*mU | |
| WV- | 199 | UCUCUUUCCAUGGAAG | fU*fC*fU*fC*fU*fU*fU*fC*fC*fA*fU*fG | XXXXX XXXXX XXXXX |
| 22340 | | GUUCUAAACCAUCC | *fG*fA*fA*fG*fG*mU*mU*mC*mU*mA* | XXXXX XXXXX XXXX |
| | | | mA*mA*rC*rC*rA*mU*mC*mC | |
| WV- | 200 | UCUCUUUCCAUGGAAG | fU*fC*fU*fC*fU*fU*fU*fC*fC*fA*fU*fG | XXXXX XXXXX XXXXX |
| 22341 | | GUUCUAAACCAUC | *fG*fA*fA*fG*fG*mU*mU*mC*mU*mA* | XXXXX XXXXX XXX |
| | | | mA*mA*rC*rC*rA*mU*mC | |
| WV- | 201 | CUCUUUCCAUGGAAGG | fC*fU*fC*fU*fU*fU*fC*fC*fA*fU*fG*fG | XXXXX XXXXX XXXXX |
| 22342 | | UUCUAAACCAUCCUG | *fA*fA*fG*fG*mU*mU*mC*mU*mA*mA* | XXXXX XXXXX XXXXX |
| | | | mA*rC*rC*rA*mU*mC*mC*mU*mG | |
| WV- | 202 | CUCUUUCCAUGGAAGG | fC*fU*fC*fU*fU*fU*fC*fC*fA*fU*fG*fG | XXXXX XXXXX XXXXX |
| 22343 | | UUCUAAACCAUCCU | *fA*fA*fG*fG*mU*mU*mC*mU*mA*mA* | XXXXX XXXXX XXXX |
| | | | mA*rC*rC*rA*mU*mC*mC*mU | |
| WV- | 203 | CUCUUUCCAUGGAAGG | fC*fU*fC*fU*fU*fU*fC*fC*fA*fU*fG*fG | XXXXX XXXXX XXXXX |
| 22344 | | UUCUAAACCAUCC | *fA*fA*fG*fG*mU*mU*mC*mU*mA*mA* | XXXXX XXXXX XXX |
| | | | mA*rC*rC*rA*mU*mC*mC | |
| WV- | 204 | CUCUUUCCAUGGAAGG | fC*fU*fC*fU*fU*fU*fC*fC*fA*fU*fG*fG | XXXXX XXXXX XXXXX |
| 22345 | | UUCUAAACCAUC | *fA*fA*fG*fG*mU*mU*mC*mU*mA*mA* | XXXXX XXXXX XX |
| | | | mA*rC*rC*rA*mU*mC | |
| WV- | 205 | UCUUUCCAUGGAAGGU | fU*fC*fU*fU*fU*fC*fC*fA*fU*fG*fG*fA | XXXXX XXXXX XXXXX |
| 22346 | | UCUAAACCAUCCUG | *fA*fG*fG*mU*mU*mC*mU*mA*mA*mA | XXXXX XXXXX XXXX |
| | | | *rC*rC*rA*mU*mC*mC*mU*mG | |
| WV- | 206 | UCUUUCCAUGGAAGGU | fU*fC*fU*fU*fU*fC*fC*fA*fU*fG*fG*fA | XXXXX XXXXX XXXXX |
| 22347 | | UCUAAACCAUCCU | *fA*fG*fG*mU*mU*mC*mU*mA*mA*mA | XXXXX XXXXX XXX |
| | | | *rC*rC*rA*mU*mC*mC*mU | |
| WV- | 207 | UCUUUCCAUGGAAGGU | fU*fC*fU*fU*fU*fC*fC*fA*fU*fG*fG*fA | XXXXX XXXXX XXXXX |
| 22348 | | UCUAAACCAUCC | *fA*fG*fG*mU*mU*mC*mU*mA*mA*mA | XXXXX XXXXX XX |
| | | | *rC*rC*rA*mU*mC*mC | |
| WV- | 208 | UCUUUCCAUGGAAGGU | fU*fC*fU*fU*fU*fC*fC*fA*fU*fG*fG*fA | XXXXX XXXXX XXXXX |
| 22349 | | UCUAAACCAUC | *fA*fG*fG*mU*mU*mC*mU*mA*mA*mA | XXXXX XXXXX X |
| | | | *rC*rC*rA*mU*mC | |
| WV- | 209 | CUUUCCAUGGAAGGUU | fC*fU*fU*fU*fC*fC*fA*fU*fG*fG*fA*fA | XXXXX XXXXX XXXXX |
| 22350 | | CUAAACCAUCCUG | *fG*fG*mU*mU*mC*mU*mA*mA*mA*rC | XXXXX XXXXX XXX |
| | | | *rC*rA*mU*mC*mC*mU*mG | |
| WV- | 210 | CUUUCCAUGGAAGGUU | fC*fU*fU*fU*fC*fC*fA*fU*fG*fG*fA*fA | XXXXX XXXXX XXXXX |
| 22351 | | CUAAACCAUCCU | *fG*fG*mU*mU*mC*mU*mA*mA*mA*rC | XXXXX XXXXX XX |
| | | | *rC*rA*mU*mC*mC*mU | |
| WV- | 211 | CUUUCCAUGGAAGGUU | fC*fU*fU*fU*fC*fC*fA*fU*fG*fG*fA*fA | XXXXX XXXXX XXXXX |
| 22352 | | CUAAACCAUCC | *fG*fG*mU*mU*mC*mU*mA*mA*mA*rC | XXXXX XXXXX X |
| | | | *rC*rA*mU*mC*mC | |
| WV- | 212 | CUUUCCAUGGAAGGUU | fC*fU*fU*fU*fC*fC*fA*fU*fG*fG*fA*fA | XXXXX XXXXX XXXXX |
| 22353 | | CUAAACCAUC | *fG*fG*mU*mU*mC*mU*mA*mA*mA*rC | XXXXX XXXXX |
| | | | *rC*rA*mU*mC | |
| WV- | 213 | UCUCUUUCCAUUGAAA | mU*mC*mU*rC*rU*rU*rU*rC*rC*rA*rU* | XXXXX XXXXX XXXXX |
| 22354 | | GUUCUAAACCAUCC | rU*rG*rA*rA*rA*rG*rU*rU*rC*rU*rA*rA | XXXXX XXXXX XXXX |
| | | | *rA*rC*rC*rA*mU*mC*mC | |
| WV- | 214 | UCUCUUUCCAAAGAAG | mU*mC*mU*rC*rU*rU*rU*rC*rC*rA*rA* | XXXXX XXXXX XXXXX |
| 22357 | | GUUCUAAACCAUCC | rA*rG*rA*rA*rG*rG*rU*rU*rC*rU*rA*rA | XXXXX XXXXX XXXX |
| | | | *rA*rC*rC*rA*mU*mC*mC | |
| WV- | 215 | UCUCUUUCCUUAGAAG | mU*mC*mU*rC*rU*rU*rU*rC*rC*rU*rU* | XXXXX XXXXX XXXXX |
| 22358 | | GUUCUAAACCAUCC | rA*rG*rA*rA*rG*rG*rU*rU*rC*rU*rA*rA | XXXXX XXXXX XXXX |
| | | | *rA*rC*rC*rA*mU*mC*mC | |
| WV- | 216 | UCUCUUUCGAUAGAAG | mU*mC*mU*rC*rU*rU*rU*rC*rG*rA*rU* | XXXXX XXXXX XXXXX |
| 22359 | | GUUCUAAACCAUCC | rA*rG*rA*rA*rG*rG*rU*rU*rC*rU*rA*rA | XXXXX XXXXX XXXX |
| | | | *rA*rC*rC*rA*mU*mC*mC | |
| WV- | 217 | UCUCUUUGCAUAGAAG | mU*mC*mU*rC*rU*rU*rU*rG*rC*rA*rU* | XXXXX XXXXX XXXXX |
| 22360 | | GUUCUAAACCAUCC | rA*rG*rA*rA*rG*rG*rU*rU*rC*rU*rA*rA | XXXXX XXXXX XXXX |
| | | | *rA*rC*rC*rA*mU*mC*mC | |
| WV- | 218 | UCUCUAUCCAUAGAAG | mU*mC*mU*rC*rU*rA*rU*rC*rC*rA*rU* | XXXXX XXXXX XXXXX |
| 22362 | | GUUCUAAACCAUCC | rA*rG*rA*rA*rG*rG*rU*rU*rC*rU*rA*rA | XXXXX XXXXX XXXX |
| | | | *rA*rC*rC*rA*mU*mC*mC | |
| WV- | 219 | UCUCAUUCCAUAGAAG | mU*mC*mU*rC*rA*rU*rU*rC*rC*rA*rU* | XXXXX XXXXX XXXXX |
| 22363 | | GUUCUAAACCAUCC | rA*rG*rA*rA*rG*rG*rU*rU*rC*rU*rA*rA | XXXXX XXXXX XXXX |
| | | | *rA*rC*rC*rA*mU*mC*mC | |
| WV- | 220 | UCUGUUUCCAUAGAAG | mU*mC*mU*rG*rU*rU*rU*rC*rC*rA*rU* | XXXXX XXXXX XXXXX |
| 22364 | | GUUCUAAACCAUCC | rA*rG*rA*rA*rG*rG*rU*rU*rC*rU*rA*rA | XXXXX XXXXX XXXX |
| | | | *rA*rC*rC*rA*mU*mC*mC | |
| WV- | 221 | UCUCUUUCCAUGGAAU | mU*mC*mU*rC*rU*rU*rU*rC*rC*rA*rU* | XXXXX XXXXX XXXXX |
| 22365 | | CUUCUAAACCAUCC | rG*rG*rA*rA*rU*rC*rU*rU*rC*rU*rA*rA | XXXXX XXXXX XXXX |
| | | | *rA*rC*rC*rA*mU*mC*mC | |
| WV- | 222 | UCUCUUUCCAUGGAAU | mU*mC*mU*rC*rU*rU*rU*rC*rC*rA*rU* | XXXXX XXXXX XXXXX |
| 22366 | | GAUCUAAACCAUCC | rG*rG*rA*rA*rU*rG*rA*rU*rC*rU*rA*rA | XXXXX XXXXX XXXX |
| | | | *rA*rC*rC*rA*mU*mC*mC | |
| WV- | 223 | UCUCUUUCCAUGGAAU | mU*mC*mU*rC*rU*rU*rU*rC*rC*rA*rU* | XXXXX XXXXX XXXXX |
| 22367 | | GUACUAAACCAUCC | rG*rG*rA*rA*rU*rG*rU*rA*rC*rU*rA*rA | XXXXX XXXXX XXXX |
| | | | *rA*rC*rC*rA*mU*mC*mC | |
| WV- | 224 | UCUCUUUCCAUGGAAU | mU*mC*mU*rC*rU*rU*rU*rC*rC*rA*rU* | XXXXX XXXXX XXXXX |
| 22368 | | GUUGUAAACCAUCC | rG*rG*rA*rA*rU*rG*rU*rU*rG*rU*rA*rA | XXXXX XXXXX XXXX |
| | | | *rA*rC*rC*rA*mU*mC*mC | |
| WV- | 225 | UCUCUUUCCAUGGAAU | mU*mC*mU*rC*rU*rU*rU*rC*rC*rA*rU* | XXXXX XXXXX XXXXX |
| 22369 | | GUUCAAAACCAUCC | rG*rG*rA*rA*rU*rG*rU*rU*rC*rA*rA*rA | XXXXX XXXXX XXXX |
| | | | *rA*rC*rC*rA*mU*mC*mC | |
| WV- | 226 | UCUCUUUCCAUGGAAU | mU*mC*mU*rC*rU*rU*rU*rC*rC*rA*rU* | XXXXX XXXXX XXXXX |
| 22370 | | GUUCUUAACCAUCC | rG*rG*rA*rA*rU*rG*rU*rU*rC*rU*rU*rA | XXXXX XXXXX XXXX |
| | | | *rA*rC*rC*rA*mU*mC*mC | |
| WV- | 227 | UCUCUUUCCAUGGAAU | mU*mC*mU*rC*rU*rU*rU*rC*rC*rA*rU* | XXXXX XXXXX XXXXX |
| 22371 | | GUUCUAUACCAUCC | rG*rG*rA*rA*rU*rG*rU*rU*rC*rU*rA*rU | XXXXX XXXXX XXXX |
| | | | *rA*rC*rC*rA*mU*mC*mC | |
| WV- | 228 | UCUCUUUCCAUGGAAU | mU*mC*mU*rC*rU*rU*rU*rC*rC*rA*rU* | XXXXX XXXXX XXXXX |
| 22372 | | GUUCUAAUCCAUCC | rG*rG*rA*rA*rU*rG*rU*rU*rC*rU*rA*rA | XXXXX XXXXX XXXX |
| | | | *rU*rC*rC*rA*mU*mC*mC | |
| WV- | 229 | UCUCUUUCCAAAGAUU | mU*mC*mU*rC*rU*rU*rU*rC*rC*rA*rA* | XXXXX XXXXX XXXXX |
| 22373 | | GUUCUAAACCAUCC | rA*rG*rA*rU*rU*rG*rU*rU*rC*rU*rA*rA | XXXXX XXXXX XXXX |
| | | | *rA*rC*rC*rA*mU*mC*mC | |
| WV- | 230 | UCUCUUUCCUUAGUAU | mU*mC*mU*rC*rU*rU*rU*rC*rC*rU*rU* | XXXXX XXXXX XXXXX |
| 22374 | | GUUCUAAACCAUCC | rA*rG*rU*rA*rU*rG*rU*rU*rC*rU*rA*rA | XXXXX XXXXX XXXX |
| | | | *rA*rC*rC*rA*mU*mC*mC | |
| WV- | 231 | UCUCUUUCGAUACAAU | mU*mC*mU*rC*rU*rU*rU*rC*rG*rA*rU* | XXXXX XXXXX XXXXX |
| 22375 | | GUUCUAAACCAUCC | rA*rC*rA*rA*rU*rG*rU*rU*rC*rU*rA*rA | XXXXX XXXXX XXXX |
| | | | *rA*rC*rC*rA*mU*mC*mC | |
| WV- | 232 | UCUCUUUGCAUUGAAU | mU*mC*mU*rC*rU*rU*rU*rG*rC*rA*rU* | XXXXX XXXXX XXXXX |
| 22376 | | GUUCUAAACCAUCC | rU*rG*rA*rA*rU*rG*rU*rU*rC*rU*rA*rA | XXXXX XXXXX XXXX |
| | | | *rA*rC*rC*rA*mU*mC*mC | |
| WV- | 233 | UCUCUUACCAAAGAAU | mU*mC*mU*rC*rU*rU*rA*rC*rC*rA*rA* | XXXXX XXXXX XXXXX |
| 22377 | | GUUCUAAACCAUCC | rA*rG*rA*rA*rU*rG*rU*rU*rC*rU*rA*rA | XXXXX XXXXX XXXX |
| | | | *rA*rC*rC*rA*mU*mC*mC | |
| WV- | 234 | UCUCUAUCCUUAGAAU | mU*mC*mU*rC*rU*rA*rU*rC*rC*rU*rU* | XXXXX XXXXX XXXXX |
| 22378 | | GUUCUAAACCAUCC | rA*rG*rA*rA*rU*rG*rU*rU*rC*rU*rA*rA | XXXXX XXXXX XXXX |
| | | | *rA*rC*rC*rA*mU*mC*mC | |
| WV- | 235 | UCUCAUUCGAUAGAAU | mU*mC*mU*rC*rA*rU*rU*rC*rG*rA*rU* | XXXXX XXXXX XXXXX |
| 22379 | | GUUCUAAACCAUCC | rA*rG*rA*rA*rU*rG*rU*rU*rC*rU*rA*rA | XXXXX XXXXX XXXX |
| | | | *rA*rC*rC*rA*mU*mC*mC | |
| WV- | 236 | UCUGUUUGCAUAGAAU | mU*mC*mU*rG*rU*rU*rU*rG*rC*rA*rU* | XXXXX XXXXX XXXXX |
| 22380 | | GUUCUAAACCAUCC | rA*rG*rA*rA*rU*rG*rU*rU*rC*rU*rA*rA | XXXXX XXXXX XXXX |
| | | | *rA*rC*rC*rA*mU*mC*mC | |
| WV- | 237 | UCUCUUUCCAUACAAU | mU*mC*mU*rC*rU*rU*rU*rC*rC*rA*rU* | XXXXX XXXXX XXXXX |
| 22381 | | CUUCUAAACCAUCC | rA*rC*rA*rA*rU*rC*rU*rU*rC*rU*rA*rA | XXXXX XXXXX XXXX |
| | | | *rA*rC*rC*rA*mU*mC*mC | |
| WV- | 238 | UCUCUUUCCAUAGUAU | mU*mC*mU*rC*rU*rU*rU*rC*rC*rA*rU* | XXXXX XXXXX XXXXX |
| 22382 | | GAUCUAAACCAUCC | rA*rG*rU*rA*rU*rG*rA*rU*rC*rU*rA*rA | XXXXX XXXXX XXXX |
| | | | *rA*rC*rC*rA*mU*mC*mC | |
| WV- | 239 | UCUCUUUCCAUAGAUU | mU*mC*mU*rC*rU*rU*rU*rC*rC*rA*rU* | XXXXX XXXXX XXXXX |
| 22383 | | GUACUAAACCAUCC | rA*rG*rA*rU*rU*rG*rU*rA*rC*rU*rA*rA | XXXXX XXXXX XXXX |
| | | | *rA*rC*rC*rA*mU*mC*mC | |
| WV- | 240 | UCUCUUUCCAUAGAAA | mU*mC*mU*rC*rU*rU*rU*rC*rC*rA*rU* | XXXXX XXXXX XXXXX |
| 22384 | | GUUGUAAACCAUCC | rA*rG*rA*rA*rA*rG*rU*rU*rG*rU*rA*rA | XXXXX XXXXX XXXX |
| | | | *rA*rC*rC*rA*mU*mC*mC | |
| WV- | 241 | UCUCUUUCCAUAGAAU | mU*mC*mU*rC*rU*rU*rU*rC*rC*rA*rU* | XXXXX XXXXX XXXXX |
| 22385 | | CUUCAAAACCAUCC | rA*rG*rA*rA*rU*rC*rU*rU*rC*rA*rA*rA | XXXXX XXXXX XXXX |
| | | | *rA*rC*rC*rA*mU*mC*mC | |
| WV- | 242 | UCUCUUUCCAUAGAAU | mU*mC*mU*rC*rU*rU*rU*rC*rC*rA*rU* | XXXXX XXXXX XXXXX |
| 22386 | | GAUCUUAACCAUCC | rA*rG*rA*rA*rU*rG*rA*rU*rC*rU*rU*rA | XXXXX XXXXX XXXX |
| | | | *rA*rC*rC*rA*mU*mC*mC | |
| WV- | 243 | UCUCUUUCCAUAGAAU | mU*mC*mU*rC*rU*rU*rU*rC*rC*rA*rU* | XXXXX XXXXX XXXXX |
| 22387 | | GUACUAUACCAUCC | rA*rG*rA*rA*rU*rG*rU*rA*rC*rU*rA*rU | XXXXX XXXXX XXXX |
| | | | *rA*rC*rC*rA*mU*mC*mC | |
| WV- | 244 | UCUCUUUCCAUAGAAU | mU*mC*mU*rC*rU*rU*rU*rC*rC*rA*rU* | XXXXX XXXXX XXXXX |
| 22388 | | GUUGUAAUCCAUCC | rA*rG*rA*rA*rU*rG*rU*rU*rG*rU*rA*rA | XXXXX XXXXX XXXX |
| | | | *rU*rC*rC*rA*mU*mC*mC | |
| WV- | 245 | UCUCUUUCCAUAGAAG | mU*mC*mU*rC*rU*rU*rU*rC*rC*rA*rU* | XXXXX XXXXX XXXXX |
| 22389 | | GUUCUAAACCAUCC | rA*rG*rA*rA*rG*rG*rU*rU*rC*rU*rA*rA | XXXXX XXXXX XXXX |
| | | | *rA*rC*rC*rA*mU*mC*mC | |
| WV- | 246 | UCUCUUUCCAUGGAAU | mU*mC*mU*rC*rU*rU*rU*rC*rC*rA*rU* | XXXXX XXXXX XXXXX |
| 22390 | | GUUCUAAACCAUCC | rG*rG*rA*rA*rU*rG*rU*rU*rC*rU*rA*rA | XXXXX XXXXX XXXX |
| | | | *rA*rC*rC*rA*mU*mC*mC | |
| WV- | 247 | UCUCUUUCCAUAGAAU | mU*mC*mU*rC*rU*rU*rU*rC*rC*rA*rU* | XXXXX XXXXX XXXXX |
| 22391 | | GUUCUAAACCAUCC | rA*rG*rA*rA*rU*rG*rU*rU*rC*rU*rA*rA | XXXXX XXXXX XXXX |
| | | | *rA*rC*rC*rA*mU*mC*mC | |
| WV- | 248 | UCUCUUUCCAUGGAAG | mU*mC*mU*C*rU*rU*rU*rC*rC*rA*rU* | XXXXX XXXXX XXXXX |
| 22392 | | GUUCUAAACCAUCCUG | rG*rG*rA*rA*rG*rG*rU*rU*rC*rU*rA*rA | XXXXX XXXXX XXXXX X |
| | | | *rA*rC*rC*rA*rU*rC*mC*mU*mG | |
| WV- | 249 | UCUCTUUCCAUGGAAG | mU*mC*mU*rC*T*rU*rU*rC*rC*rA*rU* | XXXXX XXXXX XXXXX |
| 22393 | | GUUCUAAACCAUCCUG | rG*rG*rA*rA*rG*rG*rU*rU*rC*rU*rA*rA | XXXXX XXXXX XXXXX X |
| | | | *rA*rC*rC*rA*rU*rC*mC*mU*mG | |
| WV- | 250 | UCUCUTUCCAUGGAAG | mU*mC*mU*rC*rU*T*rU*rC*rC*rA*rU* | XXXXX XXXXX XXXXX |
| 22394 | | GUUCUAAACCAUCCUG | rG*rG*rA*rA*rG*rG*rU*rU*rC*rU*rA*rA | XXXXX XXXXX XXXXX X |
| | | | *rA*rC*rC*rA*rU*rC*mC*mU*mG | |
| WV- | 251 | UCUCUUTCCAUGGAAG | mU*mC*mU*rC*rU*rU*T*rC*rC*rA*rU* | XXXXX XXXXX XXXXX |
| 22395 | | GUUCUAAACCAUCCUG | rG*rG*rA*rA*rG*rG*rU*rU*rC*rU*rA*rA | XXXXX XXXXX XXXXX X |
| | | | *rA*rC*rC*rA*rU*rC*mC*mU*mG | |
| WV- | 252 | UCUCUUUCCAUGGAAG | mU*mC*mU*rC*rU*rU*rU*C*rC*rA*rU* | XXXXX XXXXX XXXXX |
| 22396 | | GUUCUAAACCAUCCUG | rG*rG*rA*rA*rG*rG*rU*rU*rC*rU*rA*rA | XXXXX XXXXX XXXXX X |
| | | | *rA*rC*rC*rA*rU*rC*mC*mU*mG | |
| WV- | 253 | UCUCUUUCCAUGGAAG | mU*mC*mU*rC*rU*rU*rU*rC*C*rA*rU* | XXXXX XXXXX XXXXX |
| 22397 | | GUUCUAAACCAUCCUG | rG*rG*rA*rA*rG*rG*rU*rU*rC*rU*rA*rA | XXXXX XXXXX XXXXX X |
| | | | *rA*rC*rC*rA*rU*rC*mC*mU*mG | |
| WV- | 254 | UCUCUUUCCAUGGAAG | mU*mC*mU*rC*rU*rU*rU*rC*rC*A*rU* | XXXXX XXXXX XXXXX |
| 22398 | | GUUCUAAACCAUCCUG | rG*rG*rA*rA*rG*rG*rU*rU*rC*rU*rA*rA | XXXXX XXXXX XXXXX X |
| | | | *rA*rC*rC*rA*rU*rC*mC*mU*mG | |
| WV- | 255 | UCUCUUUCCATGGAAG | mU*mC*mU*rC*rU*rU*rU*rC*rC*rA*T* | XXXXX XXXXX XXXXX |
| 22399 | | GUUCUAAACCAUCCUG | rG*rG*rA*rA*rG*rG*rU*rU*rC*rU*rA*rA | XXXXX XXXXX XXXXX X |
| | | | *rA*rC*rC*rA*rU*rC*mC*mU*mG | |
| WV- | 256 | UCUCUUUCCAUGGAAG | mU*mC*mU*rC*rU*rU*rU*rC*rC*rA*rU* | XXXXX XXXXX XXXXX |
| 22400 | | GUUCUAAACCAUCCUG | G*rG*rA*rA*rG*rG*rU*rU*rC*rU*rA*rA | XXXXX XXXXX XXXXX X |
| | | | *rA*rC*rC*rA*rU*rC*mC*mU*mG | |
| WV- | 257 | UCUCUUUCCAUGGAAG | mU*mC*mU*rC*rU*rU*rU*rC*rC*rA*rU* | XXXXX XXXXX XXXXX |
| 22401 | | GUUCUAAACCAUCCUG | rG*G*rA*rA*rG*rG*rU*rU*rC*rU*rA*rA | XXXXX XXXXX XXXXX X |
| | | | *rA*rC*rC*rA*rU*rC*mC*mU*mG | |
| WV- | 258 | UCUCUUUCCAUGGAAG | mU*mC*mU*rC*rU*rU*rU*rC*rC*rA*rU* | XXXXX XXXXX XXXXX |
| 22402 | | GUUCUAAACCAUCCUG | rG*rG*A*rA*rG*rG*rU*rU*rC*rU*rA*rA | XXXXX XXXXX XXXXX X |
| | | | *rA*rC*rC*rA*rU*rC*mC*mU*mG | |
| WV- | 259 | UCUCUUUCCAUGGAAG | mU*mC*mU*rC*rU*rU*rU*rC*rC*rA*rU* | XXXXX XXXXX XXXXX |
| 22403 | | GUUCUAAACCAUCCUG | rG*rG*rA*A*rG*rG*rU*rU*rC*rU*rA*rA | XXXXX XXXXX XXXXX X |
| | | | *rA*rC*rC*rA*rU*rC*mC*mU*mG | |
| WV- | 260 | UCUCUUUCCAUGGAAG | mU*mC*mU*rC*rU*rU*rU*rC*rC*rA*rU* | XXXXX XXXXX XXXXX |
| 22404 | | GUUCUAAACCAUCCUG | rG*rG*rA*rA*G*rG*rU*rU*rC*rU*rA*rA | XXXXX XXXXX XXXXX X |
| | | | *rA*rC*rC*rA*rU*rC*mC*mU*mG | |
| WV- | 261 | UCUCUUUCCAUGGAAG | mU*mC*mU*rC*rU*rU*rU*rC*rC*rA*rU* | XXXXX XXXXX XXXXX |
| 22405 | | GUUCUAAACCAUCCUG | rG*rG*rA*rA*rG*G*rU*rU*rC*rU*rA*rA | XXXXX XXXXX XXXXX X |
| | | | *rA*rC*rC*rA*rU*rC*mC*mU*mG | |
| WV- | 262 | UCUCUUUCCAUGGAAG | mU*mC*mU*rC*rU*rU*rU*rC*rC*rA*rU* | XXXXX XXXXX XXXXX |
| 22406 | | GTUCUAAACCAUCCUG | rG*rG*rA*rA*rG*rG*T*rU*rC*rU*rA*rA | XXXXX XXXXX XXXXX X |
| | | | *rA*rC*rC*rA*rU*rC*mC*mU*mG | |
| WV- | 263 | UCUCUUUCCAUGGAAG | mU*mC*mU*rC*rU*rU*rU*rC*rC*rA*rU* | XXXXX XXXXX XXXXX |
| 22407 | | GUTCUAAACCAUCCUG | rG*rG*rA*rA*rG*rG*rU*T*rC*rU*rA*rA | XXXXX XXXXX XXXXX X |
| | | | *rA*rC*rC*rA*rU*rC*mC*mU*mG | |
| WV- | 264 | UCUCUUUCCAUGGAAG | mU*mC*mU*rC*rU*rU*rU*rC*rC*rA*rU* | XXXXX XXXXX XXXXX |
| 22408 | | GUUCUAAACCAUCCUG | rG*rG*rA*rA*rG*rG*rU*rU*C*rU*rA*rA | XXXXX XXXXX XXXXX X |
| | | | *rA*rC*rC*rA*rU*rC*mC*mU*mG | |
| WV- | 265 | UCUCUUUCCAUGGAAG | mU*mC*mU*rC*rU*rU*rU*rC*rC*rA*rU* | XXXXX XXXXX XXXXX |
| 22409 | | GUUCTAAACCAUCCUG | rG*rG*rA*rA*rG*rG*rU*rU*rC*T*rA*rA | XXXXX XXXXX XXXXX X |
| | | | *rA*rC*rC*rA*rU*rC*mC*mU*mG | |
| WV- | 266 | UCUCUUUCCAUGGAAG | mU*mC*mU*rC*rU*rU*rU*rC*rC*rA*rU* | XXXXX XXXXX XXXXX |
| 22410 | | GUUCUAAACCAUCCUG | rG*rG*rA*rA*rG*rG*rU*rU*rC*rU*A*rA | XXXXX XXXXX XXXXX X |
| | | | *rA*rC*rC*rA*rU*rC*mC*mU*mG | |
| WV- | 267 | UCUCUUUCCAUGGAAG | mU*mC*mU*rC*rU*rU*rU*rC*rC*rA*rU* | XXXXX XXXXX XXXXX |
| 22411 | | GUUCUAAACCAUCCUG | rG*rG*rA*rA*rG*rG*rU*rU*rC*rU*rA*A | XXXXX XXXXX XXXXX X |
| | | | *rA*rC*rC*rA*rU*rC*mC*mU*mG | |
| WV- | 268 | UCUCUUUCCAUGGAAG | mU*mC*mU*rC*rU*rU*rU*rC*rC*rA*rU* | XXXXX XXXXX XXXXX |
| 22412 | | GUUCUAAACCAUCCUG | rG*rG*rA*rA*rG*rG*rU*rU*rC*rU*rA*rA | XXXXX XXXXX XXXXX X |
| | | | *A*rC*rC*rA*rU*rC*mC*mU*mG | |
| WV- | 269 | UCUCUUUCCAUGGAAG | mU*mC*mU*rC*rU*rU*rU*rC*rC*rA*rU* | XXXXX XXXXX XXXXX |
| 22413 | | GUUCUAAACCAUCCUG | rG*rG*rA*rA*rG*rG*rU*rU*rC*rU*rA*rA | XXXXX XXXXX XXXXX X |
| | | | *rA*C*rC*rA*rU*rC*mC*mU*mG | |
| WV- | 270 | UCUCUUUCCAUGGAAG | mU*mC*mU*rC*rU*rU*rU*rC*rC*rA*rU* | XXXXX XXXXX XXXXX |
| 22414 | | GUUCUAAACCAUCCUG | rG*rG*rA*rA*rG*rG*rU*rU*rC*rU*rA*rA | XXXXX XXXXX XXXXX X |
| | | | *rA*rC*C*rA*rU*rC*mC*mU*mG | |
| WV- | 271 | UCUCUUUCCAUGGAAG | mU*mC*mU*rC*rU*rU*rU*rC*rC*rA*rU* | XXXXX XXXXX XXXXX |
| 22415 | | GUUCUAAACCAUCCUG | rG*rG*rA*rA*rG*rG*rU*rU*rC*rU*rA*rA | XXXXX XXXXX XXXXX X |
| | | | *rA*rC*rC*A*rU*rC*mC*mU*mG | |
| WV- | 272 | UCUCUUUCCAUGGAAG | mU*mC*mU*rC*rU*rU*rU*rC*rC*rA*rU* | XXXXX XXXXX XXXXX |
| 22417 | | GUUCUAAACCAUCCUG | rG*rG*rA*rA*rG*rG*rU*rU*rC*rU*rA*rA | XXXXX XXXXX XXXXX X |
| | | | *rA*rC*rC*rA*rU*C*mC*mU*mG | |
| WV- | 273 | UCUCUUUCCAUGGAAG | mU*mC*mU*rC*rU*rU*rU*rC*rC*rA*rU* | XXXXX XXXXX XXXXX |
| 22418 | | GUUCUAAACCAUCCUG | rG*rG*rA*rA*rG*rG*rU*rU*rC*rU*rA*rA | XXXXX XXXXX XXXXX X |
| | | | *rA*C*C*A*rU*rC*mC*mU*mG | |
| WV- | 274 | UCUCUUUCCAUGGAAG | mU*mC*mU*rC*rU*rU*rU*rC*rC*rA*rU* | XXXXX XXXXX XXXXX |
| 22420 | | GUUCUAAACCAUCCUG | rG*rG*rA*rA*rG*rG*rU*rU*rC*rU*rA*A | XXXXX XXXXX XXXXX X |
| | | | *A*C*C*A*rU*rC*mC*mU*mG | |
| WV- | 275 | UCUCUUUCCAUGGAAG | mU*mC*mU*rC*rU*rU*rU*rC*rC*rA*rU* | XXXXX XXXXX XXXXX |
| 22421 | | GUUCUAAACCATCCUG | rG*rG*rA*rA*rG*rG*rU*rU*rC*rU*rA*A | XXXXX XXXXX XXXXX X |
| | | | *A*C*C*A*T*C*mC*mU*mG | |
| WV- | 276 | UCUCUUUCCAUGGAAG | mU*mC*mU*rC*rU*rU*rU*rC*rC*rA*rU* | XXXXX XXXXX XXXXX |
| 22422 | | GUUCUAAACCAUCCUG | rG*rG*rA*rA*rG*rG*rU*rU*rC*rU*rA*rA | XXXXX XXXXX XXXXX X |
| | | | *rA*C*C*fA*rU*rC*mC*mU*mG | |
| WV- | 277 | UCUCUUUCCAUGGAAG | mU*mC*mU*rC*rU*rU*rU*rC*rC*rA*rU* | XXXXX XXXXX XXXXX |
| 22423 | | GUUCUAAACCATCCUG | rG*rG*rA*rA*rG*rG*rU*rU*rC*rU*rA*rA | XXXXX XXXXX XXXXX X |
| | | | *rA*C*C*fA*T*C*mC*mU*mG | |
| WV- | 278 | UCUCUUUCCAUGGAAG | mU*mC*mU*rC*rU*rU*rU*rC*rC*rA*rU* | XXXXX XXXXX XXXXX |
| 22424 | | GUUCUAAACCAUCCUG | rG*rG*rA*rA*rG*rG*rU*rU*rC*rU*rA*A | XXXXX XXXXX XXXXX X |
| | | | *A*C*C*fA*rU*rC*mC*mU*mG | |
| WV- | 279 | UCUCUUUCCAUGGAAG | mU*mC*mU*rC*rU*rU*rU*rC*rC*rA*rU* | XXXXX XXXXX XXXXX |
| 22425 | | GUUCUAAACCATCCUG | rG*rG*rA*rA*rG*rG*rU*rU*rC*rU*rA*A | XXXXX XXXXX XXXXX X |
| | | | *A*C*C*fA*T*C*mC*mU*mG | |
| WV- | 280 | UCUCUUUCCAUGGAAG | mU*mC*mU*rC*rU*rU*rU*rC*rC*rA*rU* | XXXXX XXXXX XXXXX |
| 22426 | | GUUCUAAACCAUCCUG | rG*rG*rA*rA*rG*rG*rU*rU*rC*rU*rA*rA | XXXXX XXXXX XXXXX X |
| | | | *rA*C*fC*A*rU*rC*mC*mU*mG | |
| WV- | 281 | UCUCUUUCCAUGGAAG | mU*mC*mU*rC*rU*rU*rU*rC*rC*rA*rU* | XXXXX XXXXX XXXXX |
| 22427 | | GUUCUAAACCATCCUG | rG*rG*rA*rA*rG*rG*rU*rU*rC*rU*rA*rA | XXXXX XXXXX XXXXX X |
| | | | *rA*C*fC*A*T*C*mC*mU*mG | |
| WV- | 282 | UCUCUUUCCAUGGAAG | mU*mC*mU*rC*rU*rU*rU*rC*rC*rA*rU* | XXXXX XXXXX XXXXX |
| 22429 | | GUUCUAAACCATCCUG | rG*rG*rA*rA*rG*rG*rU*rU*rC*rU*rA*A | XXXXX XXXXX XXXXX X |
| | | | *A*C*fC*A*T*C*mC*mU*mG | |
| WV- | 283 | UCUCUUUCCAUGGAAG | mU*mC*mU*rC*rU*rU*rU*rC*rC*rA*rU* | XXXXX XXXXX XXXXX |
| 22430 | | GUUCUAAACCAUCCUG | rG*rG*rA*rA*rG*rG*rU*rU*rC*rU*rA*rA | XXXXX XXXXX XXXXX X |
| | | | *rA*C*fC*fA*rU*rC*mC*mU*mG | |
| WV- | 284 | UCUCUUUCCAUGGAAG | mU*mC*mU*rC*rU*rU*rU*rC*rC*rA*rU* | XXXXX XXXXX XXXXX |
| 22431 | | GUUCUAAACCATCCUG | rG*rG*rA*rA*rG*rG*rU*rU*rC*rU*rA*rA | XXXXX XXXXX XXXXX X |
| | | | *rA*C*fC*fA*T*C*mC*mU*mG | |
| WV- | 285 | UCUCUUUCCAUGGAAG | mU*mC*mU*rC*rU*rU*rU*rC*rC*rA*rU* | XXXXX XXXXX XXXXX |
| 22432 | | GUUCUAAACCAUCCUG | rG*rG*rA*rA*rG*rG*rU*rU*rC*rU*rA*A | XXXXX XXXXX XXXXX X |
| | | | *A*C*fC*fA*rU*rC*mC*mU*mG | |
| WV- | 286 | UCUCUUUCCAUGGAAG | mU*mC*mU*rC*rU*rU*rU*rC*rC*rA*rU* | XXXXX XXXXX XXXXX |
| 22433 | | GUUCUAAACCATCCUG | rG*rG*rA*rA*rG*rG*rU*rU*rC*rU*rA*A | XXXXX XXXXX XXXXX X |
| | | | *A*C*fC*fA*T*C*mC*mU*mG | |
| WV- | 287 | UCUCUUUCCAUGGAAG | mU*mC*mU*rC*rU*rU*rU*rC*rC*rA*rU* | XXXXX XXXXX XXXXX |
| 22434 | | GUUCUAAACCAUCCUG | rG*rG*rA*rA*rG*rG*rU*rU*rC*rU*rA*rA | XXXXX XXXXX XXXXX X |
| | | | *rA*fC*C*A*rU*rC*mC*mU*mG | |
| WV- | 288 | UCUCUUUCCAUGGAAG | mU*mC*mU*rC*rU*rU*rU*rC*rC*rA*rU* | XXXXX XXXXX XXXXX |
| 22435 | | GUUCUAAACCATCCUG | rG*rG*rA*rA*rG*rG*rU*rU*rC*rU*rA*rA | XXXXX XXXXX XXXXX X |
| | | | *rA*fC*C*A*T*C*mC*mU*mG | |
| WV- | 289 | UCUCUUUCCAUGGAAG | mU*mC*mU*rC*rU*rU*rU*rC*rC*rA*rU* | XXXXX XXXXX XXXXX |
| 22436 | | GUUCUAAACCAUCCUG | rG*rG*rA*rA*rG*rG*rU*rU*rC*rU*rA*A | XXXXX XXXXX XXXXX X |
| | | | *A*fC*C*A*rU*rC*mC*mU*mG | |
| WV- | 290 | UCUCUUUCCAUGGAAG | mU*mC*mU*rC*rU*rU*rU*rC*rC*rA*rU* | XXXXX XXXXX XXXXX |
| 22438 | | GUUCUAAACCAUCCUG | rG*rG*rA*rA*rG*rG*rU*rU*rC*rU*rA*rA | XXXXX XXXXX XXXXX X |
| | | | *rA*fC*C*fA*rU*rC*mC*mU*mG | |
| WV- | 291 | UCUCUUUCCAUGGAAG | mU*mC*mU*rC*rU*rU*rU*rC*rC*rA*rU* | XXXXX XXXXX XXXXX |
| 22439 | | GUUCUAAACCATCCUG | rG*rG*rA*rA*rG*rG*rU*rU*rC*rU*rA*rA | XXXXX XXXXX XXXXX X |
| | | | *rA*fC*C*fA*T*C*mC*mU*mG | |
| WV- | 292 | UCUCUUUCCAUGGAAG | mU*mC*mU*rC*rU*rU*rU*rC*rC*rA*rU* | XXXXX XXXXX XXXXX |
| 22440 | | GUUCUAAACCAUCCUG | rG*rG*rA*rA*rG*rG*rU*rU*rC*rU*rA*A | XXXXX XXXXX XXXXX X |
| | | | *A*fC*C*fA*rU*rC*mC*mU*mG | |
| WV- | 293 | UCUCUUUCCAUGGAAG | mU*mC*mU*rC*rU*rU*rU*rC*rC*rA*rU* | XXXXX XXXXX XXXXX |
| 22441 | | GUUCUAAACCATCCUG | rG*rG*rA*rA*rG*rG*rU*rU*rC*rU*rA*A | XXXXX XXXXX XXXXX X |
| | | | *A*fC*C*fA*T*C*mC*mU*mG | |
| WV- | 294 | UCUCUUUCCAUGGAAG | mU*mC*mU*rC*rU*rU*rU*rC*rC*rA*rU* | XXXXX XXXXX XXXXX |
| 22442 | | GUUCUAAACCAUCCUG | rG*rG*rA*rA*rG*rG*rU*rU*rC*rU*rA*rA | XXXXX XXXXX XXXXX X |
| | | | *rA*fC*fC*A*rU*rC*mC*mU*mG | |
| WV- | 295 | UCUCUUUCCAUGGAAG | mU*mC*mU*rC*rU*rU*rU*rC*rC*rA*rU* | XXXXX XXXXX XXXXX |
| 22443 | | GUUCUAAACCATCCUG | rG*rG*rA*rA*rG*rG*rU*rU*rC*rU*rA*rA | XXXXX XXXXX XXXXX X |
| | | | *rA*fC*fC*A*T*C*mC*mU*mG | |
| WV- | 296 | UCUCUUUCCAUGGAAG | mU*mC*mU*rC*rU*rU*rU*rC*rC*rA*rU* | XXXXX XXXXX XXXXX |
| 22444 | | GUUCUAAACCAUCCUG | rG*rG*rA*rA*rG*rG*rU*rU*rC*rU*rA*A | XXXXX XXXXX XXXXX X |
| | | | *A*fC*fC*A*rU*rC*mC*mU*mG | |
| WV- | 297 | UCUCUUUCCAUGGAAG | mU*mC*mU*rC*rU*rU*rU*rC*rC*rA*rU* | XXXXX XXXXX XXXXX |
| 22445 | | GUUCUAAACCATCCUG | rG*rG*rA*rA*rG*rG*rU*rU*rC*rU*rA*A | XXXXX XXXXX XXXXX X |
| | | | *A*fC*fC*A*T*C*mC*mU*mG | |
| WV- | 298 | UCUCUUUCCAUGGAAG | mU*mC*mU*rC*rU*rU*rU*rC*rC*rA*rU* | XXXXX XXXXX XXXXX |
| 22446 | | GUUCUAAACCAUCCUG | rG*rG*rA*rA*rG*rG*rU*rU*rC*rU*rA*rA | XXXXX XXXXX XXXXX X |
| | | | *rA*fC*fC*fA*rU*rC*mC*mU*mG | |
| WV- | 299 | UCUCUUUCCAUGGAAG | mU*mC*mU*rC*rU*rU*rU*rC*rC*rA*rU* | XXXXX XXXXX XXXXX |
| 22447 | | GUUCUAAACCATCCUG | rG*rG*rA*rA*rG*rG*rU*rU*rC*rU*rA*rA | XXXXX XXXXX XXXXX X |
| | | | *rA*fC*fC*fA*T*C*mC*mU*mG | |
| WV- | 300 | UCUCUUUCCAUGGAAG | mU*mC*mU*rC*rU*rU*rU*rC*rC*rA*rU* | XXXXX XXXXX XXXXX |
| 22448 | | GUUCUAAACCAUCCUG | rG*rG*rA*rA*rG*rG*rU*rU*rC*rU*rA*A | XXXXX XXXXX XXXXX X |
| | | | *A*fC*fC*fA*rU*rC*mC*mU*mG | |
| WV- | 301 | UCUCUUUCCAUGGAAG | mU*mC*mU*rC*rU*rU*rU*rC*rC*rA*rU* | XXXXX XXXXX XXXXX |
| 22449 | | GUUCUAAACCATCCUG | rG*rG*rA*rA*rG*rG*rU*rU*rC*rU*rA*A | XXXXX XXXXX XXXXX X |
| | | | *A*fC*fC*fA*T*C*mC*mU*mG | |
| WV- | 302 | UCUCUUUCCAUGGAAG | fU*fC*fU*fC*fU*fU*fU*fC*fC*fA*fU*fG | XXXXX XXXXX XXXXX |
| 23868 | | GUUCUAAACCAUCCUG | *fG*fA*fA*fG*mG*mU*mU*mC*mU*mA* | XXXXX XXXXX XXXXX X |
| | | | mA*mA*C*C*A*mU*mC*mC*mU*mG | |
| WV- | 303 | UCUCUUUCCAUGGAAG | fU*fC*fU*fC*fU*fU*fU*fC*fC*fA*fU*fG | XXXXX XXXXX XXXXX |
| 23869 | | GUUCUAAACCAUCCU | *fG*fA*fA*fG*mG*mU*mU*mC*mU*mA* | XXXXX XXXXX XXXXX |
| | | | mA*mA*C*C*A*mU*mC*mC*mU | |
| WV- | 304 | UCUCUUUCCAUGGAAG | fU*fC*fU*fC*fU*fU*fU*fC*fC*fA*fU*fG | XXXXX XXXXX XXXXX |
| 23870 | | GUUCUAAACCAUCC | *fG*fA*fA*fG*mG*mU*mU*mC*mU*mA* | XXXXX XXXXX XXXX |
| | | | mA*mA*C*C*A*mU*mC*mC | |
| WV- | 305 | UCUCUUUCCAUGGAAG | fU*fC*fU*fC*fU*fU*fU*fC*fC*fA*fU*fG | XXXXX XXXXX XXXXX |
| 23871 | | GUUCUAAACCAUC | *fG*fA*fA*fG*mG*mU*mU*mC*mU*mA* | XXXXX XXXXX XXX |
| | | | mA*mA*C*C*A*mU*mC | |
| WV- | 306 | CUCUUUCCAUGGAAGG | fC*fU*fC*fU*fU*fU*fC*fC*fA*fU*fG*fG | XXXXX XXXXX XXXXX |
| 23872 | | UUCUAAACCAUCCUG | *fA*fA*fG*mG*mU*mU*mC*mU*mA*mA | XXXXX XXXXX XXXXX |
| | | | *mA*C*C*A*mU*mC*mC*mU*mG | |
| WV- | 307 | CUCUUUCCAUGGAAGG | fC*fU*fC*fU*fU*fU*fC*fC*fA*fU*fG*fG | XXXXX XXXXX XXXXX |
| 23873 | | UUCUAAACCAUCCU | *fA*fA*fG*mG*mU*mU*mC*mU*mA*mA | XXXXX XXXXX XXXX |
| | | | *mA*C*C*A*mU*mC*mC*mU | |
| WV- | 308 | CUCUUUCCAUGGAAGG | fC*fU*fC*fU*fU*fU*fC*fC*fA*fU*fG*fG | XXXXX XXXXX XXXXX |
| 23874 | | UUCUAAACCAUCC | *fA*fA*fG*mG*mU*mU*mC*mU*mA*mA | XXXXX XXXXX XXX |
| | | | *mA*C*C*A*mU*mC*mC | |
| WV- | 309 | CUCUUUCCAUGGAAGG | fC*fU*fC*fU*fU*fU*fC*fC*fA*fU*fG*fG | XXXXX XXXXX XXXXX |
| 23875 | | UUCUAAACCAUC | *fA*fA*fG*mG*mU*mU*mC*mU*mA*mA | XXXXX XXXXX XX |
| | | | *mA*C*C*A*mU*mC | |
| WV- | 310 | UCUUUCCAUGGAAGGU | fU*fC*fU*fU*fU*fC*fC*fA*fU*fG*fG*fA | XXXXX XXXXX XXXXX |
| 23876 | | UCUAAACCAUCCUG | *fA*fG*mG*mU*mU*mC*mU*mA*mA*mA | XXXXX XXXXX XXXX |
| | | | *C*C*A*mU*mC*mC*mU*mG | |
| WV- | 311 | UCUUUCCAUGGAAGGU | fU*fC*fU*fU*fU*fC*fC*fA*fU*fG*fG*fA | XXXXX XXXXX XXXXX |
| 23877 | | UCUAAACCAUCCU | *fA*fG*mG*mU*mU*mC*mU*mA*mA*mA | XXXXX XXXXX XXX |
| | | | *C*C*A*mU*mC*mC*mU | |
| WV- | 312 | UCUUUCCAUGGAAGGU | fU*fC*fU*fU*fU*fC*fC*fA*fU*fG*fG*fA | XXXXX XXXXX XXXXX |
| 23878 | | UCUAAACCAUCC | *fA*fG*mG*mU*mU*mC*mU*mA*mA*mA | XXXXX XXXXX XX |
| | | | *C*C*A*mU*mC*mC | |
| WV- | 313 | UCUUUCCAUGGAAGGU | fU*fC*fU*fU*fU*fC*fC*fA*fU*fG*fG*fA | XXXXX XXXXX XXXXX |
| 23879 | | UCUAAACCAUC | *fA*fG*mG*mU*mU*mC*mU*mA*mA*mA | XXXXX XXXXX X |
| | | | *C*C*A*mU*mC | |
| WV- | 314 | CUUUCCAUGGAAGGUU | fC*fU*fU*fU*fC*fC*fA*fU*fG*fG*fA*fA | XXXXX XXXXX XXXXX |
| 23880 | | CUAAACCAUCCUG | *fG*mG*mU*mU*mC*mU*mA*mA*mA*C | XXXXX XXXXX XXX |
| | | | *C*A*mU*mC*mC*mU*mG | |
| WV- | 315 | CUUUCCAUGGAAGGUU | fC*fU*fU*fU*fC*fC*fA*fU*fG*fG*fA*fA | XXXXX XXXXX XXXXX |
| 23881 | | CUAAACCAUCCU | *fG*mG*mU*mU*mC*mU*mA*mA*mA*C | XXXXX XXXXX XX |
| | | | *C*A*mU*mC*mC*mU | |
| WV- | 316 | CUUUCCAUGGAAGGUU | fC*fU*fU*fU*fC*fC*fA*fU*fG*fG*fA*fA | XXXXX XXXXX XXXXX |
| 23882 | | CUAAACCAUCC | *fG*mG*mU*mU*mC*mU*mA*mA*mA*C | XXXXX XXXXX X |
| | | | *C*A*mU*mC*mC | |
| WV- | 317 | CUUUCCAUGGAAGGUU | fC*fU*fU*fU*fC*fC*fA*fU*fG*fG*fA*fA | XXXXX XXXXX XXXXX |
| 23883 | | CUAAACCAUC | *fG*mG*mU*mU*mC*mU*mA*mA*mA*C | XXXXX XXXXX |
| | | | *C*A*mU*mC | |
| WV- | 318 | UCUCUUUCCAUGGAAG | fU*fC*fU*fC*fU*fU*fU*fC*fC*fA*fU*fG | XXXXX XXXXX XXXXX |
| 23884 | | GUUCUAAACCAUCCUG | *fG*fA*fA*fG*mG*mU*mU*mC*mU*mA* | XXXXX XXXXX XXXXX X |
| | | | mA*mA*fC*C*A*mU*mC*mC*mU*mG | |
| WV- | 319 | UCUCUUUCCAUGGAAG | fU*fC*fU*fC*fU*fU*fU*fC*fC*fA*fU*fG | XXXXX XXXXX XXXXX |
| 23885 | | GUUCUAAACCAUCCU | *fG*fA*fA*fG*mG*mU*mU*mC*mU*mA* | XXXXX XXXXX XXXXX |
| | | | mA*mA*fC*C*A*mU*mC*mC*mU | |
| WV- | 320 | UCUCUUUCCAUGGAAG | fU*fC*fU*fC*fU*fU*fU*fC*fC*fA*fU*fG | XXXXX XXXXX XXXXX |
| 23886 | | GUUCUAAACCAUCC | *fG*fA*fA*fG*mG*mU*mU*mC*mU*mA* | XXXXX XXXXX XXXX |
| | | | mA*mA*fC*C*A*mU*mC*mC | |
| WV- | 321 | UCUCUUUCCAUGGAAG | fU*fC*fU*fC*fU*fU*fU*fC*fC*fA*fU*fG | XXXXX XXXXX XXXXX |
| 23887 | | GUUCUAAACCAUC | *fG*fA*fA*fG*mG*mU*mU*mC*mU*mA* | XXXXX XXXXX XXX |
| | | | mA*mA*fC*C*A*mU*mC | |
| WV- | 322 | CUCUUUCCAUGGAAGG | fC*fU*fC*fU*fU*fU*fC*fC*fA*fU*fG*fG | XXXXX XXXXX XXXXX |
| 23888 | | UUCUAAACCAUCCUG | *fA*fA*fG*mG*mU*mU*mC*mU*mA*mA | XXXXX XXXXX XXXXX |
| | | | *mA*fC*C*A*mU*mC*mC*mU*mG | |
| WV- | 323 | CUCUUUCCAUGGAAGG | fC*fU*fC*fU*fU*fU*fC*fC*fA*fU*fG*fG | XXXXX XXXXX XXXXX |
| 23889 | | UUCUAAACCAUCCU | *fA*fA*fG*mG*mU*mU*mC*mU*mA*mA | XXXXX XXXXX XXXX |
| | | | *mA*fC*C*A*mU*mC*mC*mU | |
| WV- | 324 | CUCUUUCCAUGGAAGG | fC*fU*fC*fU*fU*fU*fC*fC*fA*fU*fG*fG | XXXXX XXXXX XXXXX |
| 23890 | | UUCUAAACCAUCC | *fA*fA*fG*mG*mU*mU*mC*mU*mA*mA | XXXXX XXXXX XXX |
| | | | *mA*fC*C*A*mU*mC*mC | |
| WV- | 325 | CUCUUUCCAUGGAAGG | fC*fU*fC*fU*fU*fU*fC*fC*fA*fU*fG*fG | XXXXX XXXXX XXXXX |
| 23891 | | UUCUAAACCAUC | *fA*fA*fG*mG*mU*mU*mC*mU*mA*mA | XXXXX XXXXX XX |
| | | | *mA*fC*C*A*mU*mC | |
| WV- | 326 | UCUUUCCAUGGAAGGU | fU*fC*fU*fU*fU*fC*fC*fA*fU*fG*fG*fA | XXXXX XXXXX XXXXX |
| 23892 | | UCUAAACCAUCCUG | *fA*fG*mG*mU*mU*mC*mU*mA*mA*mA | XXXXX XXXXX XXXX |
| | | | *fC*C*A*mU*mC*mC*mU*mG | |
| WV- | 327 | UCUUUCCAUGGAAGGU | fU*fC*fU*fU*fU*fC*fC*fA*fU*fG*fG*fA | XXXXX XXXXX XXXXX |
| 23893 | | UCUAAACCAUCCU | *fA*fG*mG*mU*mU*mC*mU*mA*mA*mA | XXXXX XXXXX XXX |
| | | | *fC*C*A*mU*mC*mC*mU | |
| WV- | 328 | UCUUUCCAUGGAAGGU | fU*fC*fU*fU*fU*fC*fC*fA*fU*fG*fG*fA | XXXXX XXXXX XXXXX |
| 23894 | | UCUAAACCAUCC | *fA*fG*mG*mU*mU*mC*mU*mA*mA*mA | XXXXX XXXXX XX |
| | | | *fC*C*A*mU*mC*mC | |
| WV- | 329 | UCUUUCCAUGGAAGGU | fU*fC*fU*fU*fU*fC*fC*fA*fU*fG*fG*fA | XXXXX XXXXX XXXXX |
| 23895 | | UCUAAACCAUC | *fA*fG*mG*mU*mU*mC*mU*mA*mA*mA | XXXXX XXXXX X |
| | | | *fC*C*A*mU*mC | |
| WV- | 330 | CUUUCCAUGGAAGGUU | fC*fU*fU*fU*fC*fC*fA*fU*fG*fG*fA*fA | XXXXX XXXXX XXXXX |
| 23896 | | CUAAACCAUCCUG | *fG*mG*mU*mU*mC*mU*mA*mA*mA*fC | XXXXX XXXXX XXX |
| | | | *C*A*mU*mC*mC*mU*mG | |
| WV- | 331 | CUUUCCAUGGAAGGUU | fC*fU*fU*fU*fC*fC*fA*fU*fG*fG*fA*fA | XXXXX XXXXX XXXXX |
| 23897 | | CUAAACCAUCCU | *fG*mG*mU*mU*mC*mU*mA*mA*mA*fC | XXXXX XXXXX XX |
| | | | *C*A*mU*mC*mC*mU | |
| WV- | 332 | CUUUCCAUGGAAGGUU | fC*fU*fU*fU*fC*fC*fA*fU*fG*fG*fA*fA | XXXXX XXXXX XXXXX |
| 23898 | | CUAAACCAUCC | *fG*mG*mU*mU*mC*mU*mA*mA*mA*fC | XXXXX XXXXX X |
| | | | *C*A*mU*mC*mC | |
| WV- | 333 | CUUUCCAUGGAAGGUU | fC*fU*fU*fU*fC*fC*fA*fU*fG*fG*fA*fA | XXXXX XXXXX XXXXX |
| 23899 | | CUAAACCAUC | *fG*mG*mU*mU*mC*mU*mA*mA*mA*fC | XXXXX XXXXX |
| | | | *C*A*mU*mC | |
| WV- | 334 | UCUCUUUCCAUGGAAG | fU*fC*fU*fC*fU*fU*fU*fC*fC*fA*fU*fG | XXXXX XXXXX XXXXX |
| 23900 | | GUUCUAAACCAUCCUG | *fG*fA*fA*fG*mG*mU*mU*mC*mU*mA* | XXXXX XXXXX XXXXX X |
| | | | mA*mA*C*mC*A*mU*mC*mC*mU*mG | |
| WV- | 335 | UCUCUUUCCAUGGAAG | fU*fC*fU*fC*fU*fU*fU*fC*fC*fA*fU*fG | XXXXX XXXXX XXXXX |
| 23901 | | GUUCUAAACCAUCCU | *fG*fA*fA*fG*mG*mU*mU*mC*mU*mA* | XXXXX XXXXX XXXXX |
| | | | mA*mA*C*mC*A*mU*mC*mC*mU | |
| WV- | 336 | UCUCUUUCCAUGGAAG | fU*fC*fU*fC*fU*fU*fU*fC*fC*fA*fU*fG | XXXXX XXXXX XXXXX |
| 23902 | | GUUCUAAACCAUCC | *fG*fA*fA*fG*mG*mU*mU*mC*mU*mA* | XXXXX XXXXX XXXX |
| | | | mA*mA*C*mC*A*mU*mC*mC | |
| WV- | 337 | UCUCUUUCCAUGGAAG | fU*fC*fU*fC*fU*fU*fU*fC*fC*fA*fU*fG | XXXXX XXXXX XXXXX |
| 23903 | | GUUCUAAACCAUC | *fG*fA*fA*fG*mG*mU*mU*mC*mU*mA* | XXXXX XXXXX XXX |
| | | | mA*mA*C*mC*A*mU*mC | |
| WV- | 338 | CUCUUUCCAUGGAAGG | fC*fU*fC*fU*fU*fU*fC*fC*fA*fU*fG*fG | XXXXX XXXXX XXXXX |
| 23904 | | UUCUAAACCAUCCUG | *fA*fA*fG*mG*mU*mU*mC*mU*mA*mA | XXXXX XXXXX XXXXX |
| | | | *mA*C*mC*A*mU*mC*mC*mU*mG | |
| WV- | 339 | CUCUUUCCAUGGAAGG | fC*fU*fC*fU*fU*fU*fC*fC*fA*fU*fG*fG | XXXXX XXXXX XXXXX |
| 23905 | | UUCUAAACCAUCCU | *fA*fA*fG*mG*mU*mU*mC*mU*mA*mA | XXXXX XXXXX XXXX |
| | | | *mA*C*mC*A*mU*mC*mC*mU | |
| WV- | 340 | CUCUUUCCAUGGAAGG | fC*fU*fC*fU*fU*fU*fC*fC*fA*fU*fG*fG | XXXXX XXXXX XXXXX |
| 23906 | | UUCUAAACCAUCC | *fA*fA*fG*mG*mU*mU*mC*mU*mA*mA | XXXXX XXXXX XXX |
| | | | *mA*C*mC*A*mU*mC*mC | |
| WV- | 341 | CUCUUUCCAUGGAAGG | fC*fU*fC*fU*fU*fU*fC*fC*fA*fU*fG*fG | XXXXX XXXXX XXXXX |
| 23907 | | UUCUAAACCAUC | *fA*fA*fG*mG*mU*mU*mC*mU*mA*mA | XXXXX XXXXX XX |
| | | | *mA*C*mC*A*mU*mC | |
| WV- | 342 | UCUUUCCAUGGAAGGU | fU*fC*fU*fU*fU*fC*fC*fA*fU*fG*fG*fA | XXXXX XXXXX XXXXX |
| 23908 | | UCUAAACCAUCCUG | *fA*fG*mG*mU*mU*mC*mU*mA*mA*mA | XXXXX XXXXX XXXX |
| | | | *C*mC*A*mU*mC*mC*mU*mG | |
| WV- | 343 | UCUUUCCAUGGAAGGU | fU*fC*fU*fU*fU*fC*fC*fA*fU*fG*fG*fA | XXXXX XXXXX XXXXX |
| 23909 | | UCUAAACCAUCCU | *fA*fG*mG*mU*mU*mC*mU*mA*mA*mA | XXXXX XXXXX XXX |
| | | | *C*mC*A*mU*mC*mC*mU | |
| WV- | 344 | UCUUUCCAUGGAAGGU | fU*fC*fU*fU*fU*fC*fC*fA*fU*fG*fG*fA | XXXXX XXXXX XXXXX |
| 23910 | | UCUAAACCAUCC | *fA*fG*mG*mU*mU*mC*mU*mA*mA*mA | XXXXX XXXXX XX |
| | | | *C*mC*A*mU*mC*mC | |
| WV- | 345 | UCUUUCCAUGGAAGGU | fU*fC*fU*fU*fU*fC*fC*fA*fU*fG*fG*fA | XXXXX XXXXX XXXXX |
| 23911 | | UCUAAACCAUC | *fA*fG*mG*mU*mU*mC*mU*mA*mA*mA | XXXXX XXXXX X |
| | | | *C*mC*A*mU*mC | |
| WV- | 346 | CUUUCCAUGGAAGGUU | fC*fU*fU*fU*fC*fC*fA*fU*fG*fG*fA*fA | XXXXX XXXXX XXXXX |
| 23912 | | CUAAACCAUCCUG | *fG*mG*mU*mU*mC*mU*mA*mA*mA*C | XXXXX XXXXX XXX |
| | | | *mC*A*mU*mC*mC*mU*mG | |
| WV- | 347 | CUUUCCAUGGAAGGUU | fC*fU*fU*fU*fC*fC*fA*fU*fG*fG*fA*fA | XXXXX XXXXX XXXXX |
| 23913 | | CUAAACCAUCCU | *fG*mG*mU*mU*mC*mU*mA*mA*mA*C | XXXXX XXXXX XX |
| | | | *mC*A*mU*mC*mC*mU | |
| WV- | 348 | CUUUCCAUGGAAGGUU | fC*fU*fU*fU*fC*fC*fA*fU*fG*fG*fA*fA | XXXXX XXXXX XXXXX |
| 23914 | | CUAAACCAUCC | *fG*mG*mU*mU*mC*mU*mA*mA*mA*C | XXXXX XXXXX X |
| | | | *mC*A*mU*mC*mC | |
| WV- | 349 | CUUUCCAUGGAAGGUU | fC*fU*fU*fU*fC*fC*fA*fU*fG*fG*fA*fA | XXXXX XXXXX XXXXX |
| 23915 | | CUAAACCAUC | *fG*mG*mU*mU*mC*mU*mA*mA*mA*C | XXXXX XXXXX |
| | | | *mC*A*mU*mC | |
| WV- | 350 | CUCUUUCCAUGGAAGG | fC*SfU*SfC*SfU*SfU*SfU*SfC*SfC*SfA* | SSSSS SSSSS SSSSS SSSSS |
| 24111 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 351 | CUCUUUCCAUGGAAGG | fC*RfU*SfC*SfU*SfU*SfU*SfC*SfC*SfA* | RSSSS SSSSS SSSSS SSSSS |
| 24112 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 352 | CUCUUUCCAUGGAAGG | fC*SfU*RfC*SfU*SfU*SfU*SfC*SfC*SfA* | SRSSS SSSSS SSSSS SSSSS |
| 24113 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 353 | CUCUUUCCAUGGAAGG | fC*SfU*SfC*RfU*SfU*SfU*SfC*SfC*SfA* | SSRSS SSSSS SSSSS SSSSS |
| 24114 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 354 | CUCUUUCCAUGGAAGG | fC*SfU*SfC*SfU*RfU*SfU*SfC*SfC*SfA* | SSSRS SSSSS SSSSS SSSSS |
| 24115 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 355 | CUCUUUCCAUGGAAGG | fC*SfU*SfC*SfU*SfU*RfU*SfC*SfC*SfA* | SSSSR SSSSS SSSSS SSSSS |
| 24116 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 356 | CUCUUUCCAUGGAAGG | fC*SfU*SfC*SfU*SfU*SfU*RfC*SfC*SfA* | SSSSS RSSSS SSSSS SSSSS |
| 24117 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 357 | CUCUUUCCAUGGAAGG | fC*SfU*SfC*SfU*SfU*SfU*SfC*RfC*SfA* | SSSSS SRSSS SSSSS SSSSS |
| 24118 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 358 | CUCUUUCCAUGGAAGG | fC*SfU*SfC*SfU*SfU*SfU*SfC*SfC*RfA* | SSSSS SSRSS SSSSS SSSSS |
| 24119 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 359 | CUCUUUCCAUGGAAGG | fC*SfU*SfC*SfU*SfU*SfU*SfC*SfC*SfA* | SSSSS SSSRS SSSSS SSSSS |
| 24120 | | UUCUAAACCAUCCU | RfU*SfG*SfG*SfA*SfA*SfG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 360 | CUCUUUCCAUGGAAGG | fC*SfU*SfC*SfU*SfU*SfU*SfC*SfC*SfA* | SSSSS SSSSR SSSSS SSSSS |
| 24121 | | UUCUAAACCAUCCU | SfU*RfG*SfG*SfA*SfA*SfG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 361 | CUCUUUCCAUGGAAGG | fC*SfU*SfC*SfU*SfU*SfU*SfC*SfC*SfA* | SSSSS SSSSS RSSSS SSSSS |
| 24122 | | UUCUAAACCAUCCU | SfU*SfG*RfG*SfA*SfA*SfG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 362 | CUCUUUCCAUGGAAGG | fC*SfU*SfC*SfU*SfU*SfU*SfC*SfC*SfA* | SSSSS SSSSS SRSSS SSSSS |
| 24123 | | UUCUAAACCAUCCU | SfU*SfG*SfG*RfA*SfA*SfG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 363 | CUCUUUCCAUGGAAGG | fC*SfU*SfC*SfU*SfU*SfU*SfC*SfC*SfA* | SSSSS SSSSS SSRSS SSSSS |
| 24124 | | UUCUAAACCAUCCU | SfU*SfG*SfG*SfA*RfA*SfG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 364 | CUCUUUCCAUGGAAGG | fC*SfU*SfC*SfU*SfU*SfU*SfC*SfC*SfA* | SSSSS SSSSS SSSRS SSSSS |
| 24125 | | UUCUAAACCAUCCU | SfU*SfG*SfG*SfA*SfA*RfG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 365 | CUCUUUCCAUGGAAGG | fC*SfU*SfC*SfU*SfU*SfU*SfC*SfC*SfA* | SSSSS SSSSS SSSSRSSSSS |
| 24126 | | UUCUAAACCAUCCU | SfU*SfG*SfG*SfA*SfA*SfG*RmG*SmU* | SSSSS SSSS |
| | | | SmU*SmC*SmU*SmA*SmA*SmA*SC*SC* | |
| | | | SA*SmU*SmC*SmC*SmU | |
| WV- | 366 | CUCUUUCCAUGGAAGG | fC*SfU*SfC*SfU*SfU*SfU*SfC*SfC*SfA* | SSSSS SSSSS SSSSSRSSSS |
| 24127 | | UUCUAAACCAUCCU | SfU*SfG*SfG*SfA*SfA*SfG*SmG*RmU* | SSSSS SSSS |
| | | | SmU*SmC*SmU*SmA*SmA*SmA*SC*SC* | |
| | | | SA*SmU*SmC*SmC*SmU | |
| WV- | 367 | CUCUUUCCAUGGAAGG | fC*SfU*SfC*SfU*SfU*SfU*SfC*SfC*SfA* | SSSSS SSSSS SSSSS SRSSS |
| 24128 | | UUCUAAACCAUCCU | SfU*SfG*SfG*SfA*SfA*SfG*SmG*SmU* | SSSSS SSSS |
| | | | RmU*SmC*SmU*SmA*SmA*SmA*SC*SC* | |
| | | | SA*SmU*SmC*SmC*SmU | |
| WV- | 368 | CUCUUUCCAUGGAAGG | fC*SfU*SfC*SfU*SfU*SfU*SfC*SfC*SfA* | SSSSS SSSSS SSSSS SSRSS |
| 24129 | | UUCUAAACCAUCCU | SfU*SfG*SfG*SfA*SfA*SfG*SmG*SmU*SmU | SSSSS SSSS |
| | | | *RmC*SmU*SmA*SmA*SmA*SC*SC*SA* | |
| | | | SmU*SmC*SmC*SmU | |
| WV- | 369 | CUCUUUCCAUGGAAGG | fC*SfU*SfC*SfU*SfU*SfU*SfC*SfC*SfA* | SSSSS SSSSS SSSSS SSSRS |
| 24130 | | UUCUAAACCAUCCU | SfU*SfG*SfG*SfA*SfA*SfG*SmG*SmU*SmU | SSSSS SSSS |
| | | | *SmC*RmU*SmA*SmA*SmA*SC*SC*SA* | |
| | | | SmU*SmC*SmC*SmU | |
| WV- | 370 | CUCUUUCCAUGGAAGG | fC*SfU*SfC*SfU*SfU*SfU*SfC*SfC*SfA* | SSSSS SSSSS SSSSS SSSSR |
| 24131 | | UUCUAAACCAUCCU | SfU*SfG*SfG*SfA*SfA*SfG*SmG*SmU*SmU | SSSSS SSSS |
| | | | *SmC*SmU*RmA*SmA*SmA*SC*SC*SA* | |
| | | | SmU*SmC*SmC*SmU | |
| WV- | 371 | CUCUUUCCAUGGAAGG | fC*SfU*SfC*SfU*SfU*SfU*SfC*SfC*SfA* | SSSSS SSSSS SSSSS SSSSS |
| 24132 | | UUCUAAACCAUCCU | SfU*SfG*SfG*SfA*SfA*SfG*SmG*SmU*SmU | RSSSS SSSS |
| | | | *SmC*SmU*SmA*RmA*SmA*SC*SC*SA* | |
| | | | SmU*SmC*SmC*SmU | |
| WV- | 372 | CUCUUUCCAUGGAAGG | fC*SfU*SfC*SfU*SfU*SfU*SfC*SfC*SfA* | SSSSS SSSSS SSSSS SSSSS |
| 24133 | | UUCUAAACCAUCCU | SfU*SfG*SfG*SfA*SfA*SfG*SmG*SmU*SmU | SRSSS SSSS |
| | | | *SmC*SmU*SmA*SmA*RmA*SC*SC*SA* | |
| | | | SmU*SmC*SmC*SmU | |
| WV- | 373 | CUCUUUCCAUGGAAGG | fC*SfU*SfC*SfU*SfU*SfU*SfC*SfC*SfA* | SSSSS SSSSS SSSSS SSSSS |
| 24134 | | UUCUAAACCAUCCU | SfU*SfG*SfG*SfA*SfA*SfG*SmG*SmU*SmU | SSRSS SSSS |
| | | | *SmC*SmU*SmA*SmA*SmA*RC*SC*SA* | |
| | | | SmU*SmC*SmC*SmU | |
| WV- | 374 | CUCUUUCCAUGGAAGG | fC*SfU*SfC*SfU*SfU*SfU*SfC*SfC*SfA* | SSSSS SSSSS SSSSS SSSSS |
| 24135 | | UUCUAAACCAUCCU | SfU*SfG*SfG*SfA*SfA*SfG*SmG*SmU*SmU | SSSRSSSSS |
| | | | *SmC*SmU*SmA*SmA*SmA*SC*RC*SA* | |
| | | | SmU*SmC*SmC*SmU | |
| WV- | 375 | CUCUUUCCAUGGAAGG | fC*SfU*SfC*SfU*SfU*SfU*SfC*SfC*SfA* | SSSSS SSSSS SSSSS SSSSS |
| 24136 | | UUCUAAACCAUCCU | SfU*SfG*SfG*SfA*SfA*SfG*SmG*SmU*SmU | SSSSRSSSS |
| | | | *SmC*SmU*SmA*SmA*SmA*SC*SC*RA* | |
| | | | SmU*SmC*SmC*SmU | |
| WV- | 376 | CUCUUUCCAUGGAAGG | fC*SfU*SfC*SfU*SfU*SfU*SfC*SfC*SfA* | SSSSS SSSSS SSSSS SSSSS |
| 24137 | | UUCUAAACCAUCCU | SfU*SfG*SfG*SfA*SfA*SfG*SmG*SmU*SmU | SSSSS RSSS |
| | | | *SmC*SmU*SmA*SmA*SmA*SC*SC*SA* | |
| | | | RmU*SmC*SmC*SmU | |
| WV- | 377 | CUCUUUCCAUGGAAGG | fC*SfU*SfC*SfU*SfU*SfU*SfC*SfC*SfA* | SSSSS SSSSS SSSSS SSSSS |
| 24138 | | UUCUAAACCAUCCU | SfU*SfG*SfG*SfA*SfA*SfG*SmG*SmU*SmU | SSSSS SRSS |
| | | | *SmC*SmU*SmA*SmA*SmA*SC*SC*SA* | |
| | | | SmU*RmC*SmC*SmU | |
| WV- | 378 | CUCUUUCCAUGGAAGG | fC*SfU*SfC*SfU*SfU*SfU*SfC*SfC*SfA* | SSSSS SSSSS SSSSS SSSSS |
| 24139 | | UUCUAAACCAUCCU | SfU*SfG*SfG*SfA*SfA*SfG*SmG*SmU*SmU | SSSSS SSRS |
| | | | *SmC*SmU*SmA*SmA*SmA*SC*SC*SA* | |
| | | | SmU*SmC*RmC*SmU | |
| WV- | 379 | CUCUUUCCAUGGAAGG | fC*SfU*SfC*SfU*SfU*SfU*SfC*SfC*SfA* | SSSSS SSSSS SSSSS SSSSS |
| 24140 | | UUCUAAACCAUCCU | SfU*SfG*SfG*SfA*SfA*SfG*SmG*SmU*SmU | SSSSS SSSR |
| | | | *SmC*SmU*SmA*SmA*SmA*SC*SC*SA* | |
| | | | SmU*SmC*SmC*RmU | |
| WV- | 380 | CUCUUUCCAUGGAAGG | fC*RfU*RfC*RfU*RfU*RfU*RfC*RfC*RfA* | RRRRR RRRRR RRRRR |
| 24141 | | UUCUAAACCAUCCU | RfU*RfG*RfG*RfA*RfA*RfG*RmG*RmU* | RRRRR RRRRR RRRR |
| | | | RmU*RmC*RmU*RmA*RmA*RmA*RC*RC* | |
| | | | RA*RmU*RmC*RmC*RmU | |
| WV- | 381 | CUCUUUCCAUGGAAGG | fC*RfU*RfC*RfU*RfU*RfU*RfC*RfC*RfA* | RRRRR RRRRR SSSSS |
| 24142 | | UUCUAAACCAUCCU | RfU*RfG*SfG*SfA*SfA*SfG*SmG*SmU* | SSSSS SSSSS SSSS |
| | | | SmU*SmC*SmU*SmA*SmA*SmA*SC*SC* | |
| | | | SA*SmU*SmC*SmC*SmU | |
| WV- | 382 | CUCUUUCCAUGGAAGG | fC*RfU*RfC*RfU*RfU*RfU*RfC*RfC*RfA* | RRRRR RRRRR RRRRS |
| 24143 | | UUCUAAACCAUCCU | RfU*RfG*RfG*RfA*RfA*RfG*SmG*SmU* | SSSSS SSSSS SSSS |
| | | | SmU*SmC*SmU*SmA*SmA*SmA*SC*SC* | |
| | | | SA*SmU*SmC*SmC*SmU | |
| WV- | 383 | CUCUUUCCAUGGAAGG | fC*SfU*SfC*SfU*SfU*SfU*SfC*SfC*SfA* | SSSSS SSSSS RRRRS SSSSS |
| 24144 | | UUCUAAACCAUCCU | SfU*SfG*RfG*RfA*RfA*RfG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 384 | CUCUUUCCAUGGAAGG | fC*SfU*SfC*SfU*SfU*SfU*SfC*SfC*SfA* | SSSSS SSSSS RRRRR |
| 24145 | | UUCUAAACCAUCCU | SfU*SfG*RfG*RfA*RfA*RfG*RmG*RmU* | RRRRR RRRRR RRRR |
| | | | RmU*RmC*RmU*RmA*RmA*RmA*RC*RC* | |
| | | | RA*RmU*RmC*RmC*RmU | |
| WV- | 385 | CUCUUUCCAUGGAAGG | fC*SfU*SfC*SfU*SfU*SfU*SfC*SfC*SfA* | SSSSS SSSSS SSSSR RRRRR |
| 24146 | | UUCUAAACCAUCCU | SfU*SfG*SfG*SfA*SfA*SfG*RmG*RmU* | RRRRR RRRR |
| | | | RmU*RmC*RmU*RmA*RmA*RmA*RC*RC* | |
| | | | RA*RmU*RmC*RmC*RmU | |
| WV- | 386 | CUCUUUCCAUGGAAGG | fC*RfU*RfC*RfU*RfU*RfU*RfC*RfC*RfA* | RRRRR RRRRR SSSSR |
| 24147 | | UUCUAAACCAUCCU | RfU*RfG*SfG*SfA*SfA*SfG*RmG*RmU* | RRRRR RRRRR RRRR |
| | | | RmU*RmC*RmU*RmA*RmA*RmA*RC*RC* | |
| | | | RA*RmU*RmC*RmC*RmU | |
| WV- | 387 | CUCUUUCCAUGGAAGG | fC*SfU*SfC*SfU*SfU*SfU*SfC*SfC*SfA* | SSSSS SSSSS SSSSS |
| 24148 | | UUCUAAACCAUCCU | SfU*SfG*SfG*SfA*SfA*SfG*SmG*SmU* | SRRSSSSSSSSSSS |
| | | | RmU*RmC*SmU*SmA*SmA*SmA*SC*SC* | |
| | | | SA*SmU*SmC*SmC*SmU | |
| WV- | 388 | CUCUUUCCAUGGAAGG | fC*SfU*SfC*SfU*SfU*SfU*SfC*SfC*SfA* | SSSSS SSSSS SSSSS SSSSS |
| 24149 | | UUCUAAACCAUCCU | SfU*SfG*SfG*SfA*SfA*SfG*SmG*SmU*SmU | SSSSR RRRR |
| | | | *SmC*SmU*SmA*SmA*SmA*SC*SC*RA* | |
| | | | RmU*RmC*RmC*RmU | |
| WV- | 389 | CUCUUUCCAUGGAAGG | fC*SfU*SfC*SfU*SfU*SfU*SfC*SfC*SfA* | SSSSS SSSSS SSSSS SRRSS |
| 24150 | | UUCUAAACCAUCCU | SfU*SfG*SfG*SfA*SfA*SfG*SmG*SmU* | SSSSRRRRR |
| | | | RmU*RmC*SmU*SmA*SmA*SmA*SC*SC* | |
| | | | RA*RmU*RmC*RmC*RmU | |
| WV- | 390 | CUCUUUCCAUGGAAGG | fC*RfU*RfC*RfU*RfU*RfU*RfC*RfC*RfA* | RRRRR RRRRR RRRRR |
| 24151 | | UUCUAAACCAUCCU | RfU*RfG*RfG*RfA*RfA*RfG*RmG*RmU* | RSSRR RRRRR RRRR |
| | | | SmU*SmC*RmU*RmA*RmA*RmA*RC*RC* | |
| | | | RA*RmU*RmC*RmC*RmU | |
| WV- | 391 | CUCUUUCCAUGGAAGG | fC*RfU*RfC*RfU*RfU*RfU*RfC*RfC*RfA* | RRRRR RRRRR RRRRR |
| 24152 | | UUCUAAACCAUCCU | RfU*RfG*RfG*RfA*RfA*RfG*RmG*RmU* | RRRRR RRRRS SSSS |
| | | | RmU*RmC*RmU*RmA*RmA*RmA*RC*RC* | |
| | | | SA*SmU*SmC*SmC*SmU | |
| WV- | 392 | CUCUUUCCAUGGAAGG | fC*RfU*RfC*RfU*RfU*RfU*RfC*RfC*RfA* | RRRRR RRRRR RRRRR |
| 24153 | | UUCUAAACCAUCCU | RfU*RfG*RfG*RfA*RfA*RfG*RmG*RmU* | RSSRR RRRRS SSSS |
| | | | SmU*SmC*RmU*RmA*RmA*RmA*RC*RC* | |
| | | | SA*SmU*SmC*SmC*SmU | |
| WV- | 393 | CUCUUUCCAUGGAAGG | fC*SfU*SfC*SfU*SfU*SfU*SfC*SfC*SfA* | SSSSS SSSSS SSSSS SSSSS |
| 24154 | | UUCUAAACCAUCCU | SfU*SfG*SfG*SfA*SfA*SfG*SmG*SmU*SmU | SSSRRSSSS |
| | | | *SmC*SmU*SmA*SmA*SmA*SC*RC*RA* | |
| | | | SmU*SmC*SmC*SmU | |
| WV- | 394 | CUCUUUCCAUGGAAGG | fC*SfU*SfC*SfU*SfU*SfU*SfC*SfC*SfA* | SSSSS SSSSS SSSSS SSSSS |
| 24155 | | UUCUAAACCAUCCU | SfU*SfG*SfG*SfA*SfA*SfG*SmG*SmU*SmU | SSRRRSSSS |
| | | | *SmC*SmU*SmA*SmA*SmA*RC*RC*RA* | |
| | | | SmU*SmC*SmC*SmU | |
| WV- | 395 | CUCUUUCCAUGGAAGG | fC*SfU*SfC*SfU*SfU*SfU*SfC*SfC*SfA* | SSSSS SSSSS SSSSS SSSSS |
| 24156 | | UUCUAAACCAUCCU | SfU*SfG*SfG*SfA*SfA*SfG*SmG*SmU*SmU | SSSRRRSSS |
| | | | *SmC*SmU*SmA*SmA*SmA*SC*RC*RA* | |
| | | | RmU*SmC*SmC*SmU | |
| WV- | 396 | CUCUUUCCAUGGAAGG | fC*SfU*SfC*SfU*SfU*SfU*SfC*SfC*SfA* | SSSSS SSSSS SSSSS SSSSS |
| 24157 | | UUCUAAACCAUCCU | SfU*SfG*SfG*SfA*SfA*SfG*SmG*SmU*SmU | SSRRS SSSS |
| | | | *SmC*SmU*SmA*SmA*SmA*RC*RC*SA* | |
| | | | SmU*SmC*SmC*SmU | |
| WV- | 397 | CUCUUUCCAUGGAAGG | fC*SfU*SfC*SfU*SfU*SfU*SfC*SfC*SfA* | SSSSS SSSSS SSSSS SSSSS |
| 24158 | | UUCUAAACCAUCCU | SfU*SfG*SfG*SfA*SfA*SfG*SmG*SmU*SmU | SSSSRRSSS |
| | | | *SmC*SmU*SmA*SmA*SmA*SC*SC*RA* | |
| | | | RmU*SmC*SmC*SmU | |
| WV- | 398 | UCUCUUUCCAUGGAAG | fU*fC*fU*fC*fU*fU*fU*fC*fC*fA*fU*fG | XXXXX XXXXX XXXXX |
| 28183 | | GUUCUAAACUAUCCUG | *fG*fA*fA*fG*mG*mU*mU*mC*mU*mA* | XXXXX XXXXX XXXXX X |
| | | | mA*mA*C*b001U*A*mU*mC*mC*mU*mG | |
| WV- | 399 | UCUCUUUCCAUGGAAG | fU*fC*fU*fC*fU*fU*fU*fC*fC*fA*fU*fG | XXXXX XXXXX XXXXX |
| 28183b | | GUUCUAAACUAUCCUG | *fG*fA*fA*fG*mG*mU*mU*mC*mU*mA* | XXXXX XXXXX XXXXX X |
| | | | mA*mA*C*b001rU*A*mU*mC*mC*mU*mG | |
| WV- | 400 | CUCUUUCCAUGGAAGG | fCn001fU*SfC*SfU*SfU*SfU*SfC*SfC*SfA* | nXSSSS SSSSS SSSSS SSSSS |
| 29836 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 401 | CUCUUUCCAUGGAAGG | fC*SfUn001fC*SfU*SfU*SfU*SfC*SfC*SfA* | SnXSSS SSSSS SSSSS SSSSS |
| 29837 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 402 | CUCUUUCCAUGGAAGG | fC*SfU*SfCn001fU*SfU*SfU*SfC*SfC*SfA* | SSnXSS SSSSS SSSSS S |
| 29838 | | UUCUAAACCAUCCU | SfU*SfG*SfG*SfA*SfA*SfG*SmG*SmU*SmU | SSSSS SSSSS SSS |
| | | | *SmC*SmU*SmA*SmA*SmA*SC*SC*SA* | |
| | | | SmU*SmC*SmC*SmU | |
| WV- | 403 | CUCUUUCCAUGGAAGG | fC*SfU*SfC*SfUn001fU*SfU*SfC*SfC*SfA* | SSSnXS SSSSS SSSSS SSSSS |
| 29839 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 404 | CUCUUUCCAUGGAAGG | fC*SfU*SfC*SfU*SfUn001fU*SfC*SfC*SfA* | SSSSnX SSSSS SSSSS SSSSS |
| 29840 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 405 | CUCUUUCCAUGGAAGG | fC*SfU*SfC*SfU*SfU*SfUn001fC*SfC*SfA* | SSSSS nXSSSS SSSSS SSSSS |
| 29841 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 406 | CUCUUUCCAUGGAAGG | fC*SfU*SfC*SfU*SfU*SfU*SfCn001fC*SfA* | SSSSS SnXSSS SSSSS SSSSS |
| 29842 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 407 | CUCUUUCCAUGGAAGG | fC*SfU*SfC*SfU*SfU*SfU*SfC*SfCn001fA* | SSSSS SSnXSS SSSSS SSSSS |
| 29843 | | UUCUAAACCAUCCU | SfU*SfG*SfG*SfA*SfA*SfG*SmG*SmU*SmU | SSSSS SSSS |
| | | | *SmC*SmU*SmA*SmA*SmA*SC*SC*SA* | |
| | | | SmU*SmC*SmC*SmU | |
| WV- | 408 | CUCUUUCCAUGGAAGG | fC*SfU*SfC*SfU*SfU*SfU*SfC*SfC* | SSSSS SSSnXS SSSSS SSSSS |
| 29844 | | UUCUAAACCAUCCU | SfAn001fU*SfG*SfG*SfA*SfA*SfG*SmG*SmU | SSSSS SSSS |
| | | | *SmU*SmC*SmU*SmA*SmA*SmA*SC*SC* | |
| | | | SA*SmU*SmC*SmC*SmU | |
| WV- | 409 | CUCUUUCCAUGGAAGG | fC*SfU*SfC*SfU*SfU*SfU*SfC*SfC*SfA* | SSSSS SSSSnX SSSSS SSSSS |
| 29845 | | UUCUAAACCAUCCU | SfUn001fG*SfG*SfA*SfA*SfG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 410 | CUCUUUCCAUGGAAGG | fC*SfU*SfC*SfU*SfU*SfU*SfC*SfC*SfA* | SSSSS SSSSS nXSSSS SSSSS |
| 29846 | | UUCUAAACCAUCCU | SfU*SfGn001fG*SfA*SfA*SfG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 411 | CUCUUUCCAUGGAAGG | fC*SfU*SfC*SfU*SfU*SfU*SfC*SfC*SfA* | SSSSS SSSSS SnXSSS SSSSS |
| 29847 | | UUCUAAACCAUCCU | SfU*SfG*SfGn001fA*SfA*SfG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 412 | CUCUUUCCAUGGAAGG | fC*SfU*SfC*SfU*SfU*SfU*SfC*SfC*SfA* | SSSSS SSSSS SSnXSS SSSSS |
| 29848 | | UUCUAAACCAUCCU | SfU*SfG*SfG*SfAn001fA*SfG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 413 | CUCUUUCCAUGGAAGG | fC*SfU*SfC*SfU*SfU*SfU*SfC*SfC*SfA* | SSSSS SSSSS SSSnXS SSSSS |
| 29849 | | UUCUAAACCAUCCU | SfU*SfG*SfG*SfA*SfAn001fG*SmG*SmU* | SSSSS SSSS |
| | | | SmU*SmC*SmU*SmA*SmA*SmA*SC*SC* | |
| | | | SA*SmU*SmC*SmC*SmU | |
| WV- | 414 | CUCUUUCCAUGGAAGG | fC*SfU*SfC*SfU*SfU*SfU*SfC*SfC*SfA* | SSSSS SSSSS SSSSnX SSSSS |
| 29850 | | UUCUAAACCAUCCU | SfU*SfG*SfG*SfA*SfA*SfGn001mG*SmU* | SSSSS SSSS |
| | | | SmU*SmC*SmU*SmA*SmA*SmA*SC*SC* | |
| | | | SA*SmU*SmC*SmC*SmU | |
| WV- | 415 | CUCUUUCCAUGGAAGG | fC*SfU*SfC*SfU*SfU*SfU*SfC*SfC*SfA* | SSSSS SSSSS SSSSS nXSSSS |
| 29851 | | UUCUAAACCAUCCU | SfU*SfG*SfG*SfA*SfA*SfG*SmGn001mU* | SSSSS SSSS |
| | | | SmU*SmC*SmU*SmA*SmA*SmA*SC*SC* | |
| | | | SA*SmU*SmC*SmC*SmU | |
| WV- | 416 | CUCUUUCCAUGGAAGG | fC*SfU*SfC*SfU*SfU*SfU*SfC*SfC*SfA* | SSSSS SSSSS SSSSS SnXSSS |
| 29852 | | UUCUAAACCAUCCU | SfU*SfG*SfG*SfA*SfA*SfG*SmG* | SSSSS SSSS |
| | | | SmUn001mU*SmC*SmU*SmA*SmA*SmA*SC* | |
| | | | SC*SA*SmU*SmC*SmC*SmU | |
| WV- | 417 | CUCUUUCCAUGGAAGG | fC*SfU*SfC*SfU*SfU*SfU*SfC*SfC*SfA* | SSSSS SSSSS SSSSS SSnXSS |
| 29853 | | UUCUAAACCAUCCU | SfU*SfG*SfG*SfA*SfA*SfG*SmG*SmU* | SSSSS SSSS |
| | | | SmUn001mC*SmU*SmA*SmA*SmA*SC*SC* | |
| | | | SA*SmU*SmC*SmC*SmU | |
| WV- | 418 | CUCUUUCCAUGGAAGG | fC*SfU*SfC*SfU*SfU*SfU*SfC*SfC*SfA* | SSSSS SSSSS SSSSS SSSnXS |
| 29854 | | UUCUAAACCAUCCU | SfU*SfG*SfG*SfA*SfA*SfG*SmG*SmU*SmU | SSSSS SSSS |
| | | | *SmCn001mU*SmA*SmA*SmA*SC*SC*SA* | |
| | | | SmU*SmC*SmC*SmU | |
| WV- | 419 | CUCUUUCCAUGGAAGG | fC*SfU*SfC*SfU*SfU*SfU*SfC*SfC*SfA* | SSSSS SSSSS SSSSS SSSSnX |
| 29855 | | UUCUAAACCAUCCU | SfU*SfG*SfG*SfA*SfA*SfG*SmG*SmU*SmU | SSSSS SSSS |
| | | | *SmC*SmUn001mA*SmA*SmA*SC*SC*SA* | |
| | | | SmU*SmC*SmC*SmU | |
| WV- | 420 | CUCUUUCCAUGGAAGG | fC*SfU*SfC*SfU*SfU*SfU*SfC*SfC*SfA* | SSSSS SSSSS SSSSS SSSSS |
| 29856 | | UUCUAAACCAUCCU | SfU*SfG*SfG*SfA*SfA*SfG*SmG*SmU*SmU | nXSSSS SSSS |
| | | | *SmC*SmU*SmAn001mA*SmA*SC*SC*SA* | |
| | | | SmU*SmC*SmC*SmU | |
| WV- | 421 | CUCUUUCCAUGGAAGG | fC*SfU*SfC*SfU*SfU*SfU*SfC*SfC*SfA* | SSSSS SSSSS SSSSS SSSSS |
| 29857 | | UUCUAAACCAUCCU | SfU*SfG*SfG*SfA*SfA*SfG*SmG*SmU*SmU | SnXSSS SSSS |
| | | | *SmC*SmU*SmA*SmAn001mA*SC*SC*SA* | |
| | | | SmU*SmC*SmC*SmU | |
| WV- | 422 | CUCUUUCCAUGGAAGG | fC*SfU*SfC*SfU*SfU*SfU*SfC*SfC*SfA* | SSSSS SSSSS SSSSS SSSSS |
| 29858 | | UUCUAAACCAUCCU | SfU*SfG*SfG*SfA*SfA*SfG*SmG*SmU*SmU | SSnXSSSSSS |
| | | | *SmC*SmU*SmA*SmA*SmAn001C*SC*SA* | |
| | | | SmU*SmC*SmC*SmU | |
| WV- | 423 | CUCUUUCCAUGGAAGG | fC*SfU*SfC*SfU*SfU*SfU*SfC*SfC*SfA* | SSSSS SSSSS SSSSS SSSSS |
| 29859 | | UUCUAAACCAUCCU | SfU*SfG*SfG*SfA*SfA*SfG*SmG*SmU*SmU | SSSnXS SSSS |
| | | | *SmC*SmU*SmA*SmA*SmA*SCn001C*SA* | |
| | | | SmU*SmC*SmC*SmU | |
| WV- | 424 | CUCUUUCCAUGGAAGG | fC*SfU*SfC*SfU*SfU*SfU*SfC*SfC*SfA* | SSSSS SSSSS SSSSS SSSSS |
| 29860 | | UUCUAAACCAUCCU | SfU*SfG*SfG*SfA*SfA*SfG*SmG*SmU*SmU | SSSSnX SSSS |
| | | | *SmC*SmU*SmA*SmA*SmA*SC*SCn001A* | |
| | | | SmU*SmC*SmC*SmU | |
| WV- | 425 | CUCUUUCCAUGGAAGG | fC*SfU*SfC*SfU*SfU*SfU*SfC*SfC*SfA* | SSSSS SSSSS SSSSS SSSSS |
| 29861 | | UUCUAAACCAUCCU | SfU*SfG*SfG*SfA*SfA*SfG*SmG*SmU*SmU | SSSSS nXSSS |
| | | | *SmC*SmU*SmA*SmA*SmA*SC*SC* | |
| | | | SAn001mU*SmC*SmC*SmU | |
| WV- | 426 | CUCUUUCCAUGGAAGG | fC*SfU*SfC*SfU*SfU*SfU*SfC*SfC*SfA* | SSSSS SSSSS SSSSS SSSSS |
| 29862 | | UUCUAAACCAUCCU | SfU*SfG*SfG*SfA*SfA*SfG*SmG*SmU*SmU | SSSSS SnXSS |
| | | | *SmC*SmU*SmA*SmA*SmA*SC*SC*SA* | |
| | | | SmUn001mC*SmC*SmU | |
| WV- | 427 | CUCUUUCCAUGGAAGG | fC*SfU*SfC*SfU*SfU*SfU*SfC*SfC*SfA* | SSSSS SSSSS SSSSS |
| 29863 | | UUCUAAACCAUCCU | SfU*SfG*SfG*SfA*SfA*SfG*SmG*SmU*SmU | SSSSS |
| | | | *SmC*SmU*SmA*SmA*SmA*SC*SC*SA* | SSSSS SSnXS |
| | | | SmU*SmCn001mC*SmU | |
| WV- | 428 | CUCUUUCCAUGGAAGG | fC*SfU*SfC*SfU*SfU*SfU*SfC*SfC*SfA* | SSSSS SSSSS SSSSS |
| 29864 | | UUCUAAACCAUCCU | SfU*SfG*SfG*SfA*SfA*SfG*SmG*SmU*SmU | SSSSS |
| | | | *SmC*SmU*SmA*SmA*SmA*SC*SC*SA* | SSSSS SSSnX |
| | | | SmU*SmC*SmCn001mU | |
| WV- | 429 | CUCUUUCCAUGGAAGG | fC*SfU*SfC*SfU*SfU*SfU*SfC*SfC*SfA*SfU* | SSSSSSSSSSSSSSSSSSS |
| 29874 | | UUCUAAACCAUCCU | SfG*SfG*SfA*SfA*SfG*SmG*SmU*SmU*SmC*SmU* | SSSSSSSSSS |
| | | | SmA*SmA*SmA*SC*SrC*SA*SmU*SmC*SmC*SmU | |
| WV- | 430 | CUCUUUCCAUGGAAGG | fC*fU*fC*fU*fU*fU*fC*fC*fA*fU*fG*fG | XXXXX XXXXX XXXXX |
| 27521 | | UUCUAAACUAUCCU | *fA*fA*fG*mG*mU*mU*mC*mU*mA*mA | XXXXX XXXXX XXXX |
| | | | *mA*C*rU*A*mU*mC*mC*mU | |
| WV- | 431 | UCUCUUUCCAUGGAAG | fU*fC*fU*fC*fU*fU*fU*fC*fC*fA*fU*fG | XXXXX XXXXX XXXXX |
| 31133 | | GUUCUAAACUAUCCUG | *fG*fA*fA*fG*mG*mU*mU*mC*mU*mA* | XXXXX XXXXX XXXXX |
| | | | mA*mA*C*b002U*A*mU*mC*mC*mU*mG | X |
| WV- | 432 | UCUCUUUCCAUGGAAG | fU*fC*fU*fC*fU*fU*fU*fC*fC*fA*fU*fG | XXXXX XXXXX XXXXX |
| 31134 | | GUUCUAAACUAUCCUG | *fG*fA*fA*fG*mG*mU*mU*mC*mU*mA* | XXXXX XXXXX XXXXX |
| | | | mA*mA*C*bOO3U*A*mU*mC*mC*mU*mG | X |
| WV- | 433 | UCUCUUUCCAUGGAAG | fU*fC*fU*fC*fU*fU*fU*fC*fC*fA*fU*fG | XXXXX XXXXX XXXXX |
| 31135 | | GUUCUAAACUAUCCUG | *fG*fA*fA*fG*mG*mU*mU*mC*mU*mA* | XXXXX XXXXX XXXXX |
| | | | mA*mA*C*bOO5U*A*mU*mC*mC*mU*mG | X |
| WV- | 434 | UCUCUUUCCAUGGAAG | fU*fC*fU*fC*fU*fU*fU*fC*fC*fA*fU*fG | XXXXX XXXXX XXXXX |
| 31137 | | GUUCUAAACUAUCCUG | *fG*fA*fA*fG*mG*mU*mU*mC*mU*mA* | XXXXX XXXXX XXXXX |
| | | | mA*mA*C*b007U*A*mU*mC*mC*mU*mG | X |
| WV- | 435 | UCUCUUUCCAUGGAAG | fU*fC*fU*fC*fU*fU*fU*fC*fC*fA*fU*fG | XXXXX XXXXX XXXXX |
| 31138 | | GUUCUAAACAAUCCUG | *fG*fA*fA*fG*mG*mU*mU*mC*mU*mA* | XXXXX XXXXX XXXXX |
| | | | mA*mA*C*b001A*A*mU*mC*mC*mU*mG | X |
| WV- | 436 | CUCUUUCCAUGGAAGG | fC*fU*fC*fU*fU*fU*fC*fC*fA*fU*fG*fG | XXXXX XXXXX XXXXX |
| 31139 | | UUCUAAACUAUCCU | *fA*fA*fG*mG*mU*mU*mC*mU*mA*mA | XXXXX XXXXX XXXX |
| | | | *mA*C*b002U*A*mU*mC*mC*mU | |
| WV- | 437 | CUCUUUCCAUGGAAGG | fC*fU*fC*fU*fU*fU*fC*fC*fA*fU*fG*fG | XXXXX XXXXX XXXXX |
| 31140 | | UUCUAAACUAUCCU | *fA*fA*fG*mG*mU*mU*mC*mU*mA*mA | XXXXX XXXXX XXXX |
| | | | *mA*C*b003U*A*mU*mC*mC*mU | |
| WV- | 438 | CUCUUUCCAUGGAAGG | fC*fU*fC*fU*fU*fU*fC*fC*fA*fU*fG*fG | XXXXX XXXXX XXXXX |
| 31141 | | UUCUAAACUAUCCU | *fA*fA*fG*mG*mU*mU*mC*mU*mA*mA | XXXXX XXXXX XXXX |
| | | | *mA*C*b005U*A*mU*mC*mC*mU | |
| WV- | 439 | CUCUUUCCAUGGAAGG | fC*fU*fC*fU*fU*fU*fC*fC*fA*fU*fG*fG | XXXXX XXXXX XXXXX |
| 31142 | | UUCUAAACUAUCCU | *fA*fA*fG*mG*mU*mU*mC*mU*mA*mA | XXXXX XXXXX XXXX |
| | | | *mA*C*b006U*A*mU*mC*mC*mU | |
| WV- | 440 | CUCUUUCCAUGGAAGG | fC*fU*fC*fU*fU*fU*fC*fC*fA*fU*fG*fG | XXXXX XXXXX XXXXX |
| 31143 | | UUCUAAACUAUCCU | *fA*fA*fG*mG*mU*mU*mC*mU*mA*mA | XXXXX XXXXX XXXX |
| | | | *mA*C*b007U*A*mU*mC*mC*mU | |
| WV- | 441 | CUCUUUCCAUGGAAGG | fC*fU*fC*fU*fU*fU*fC*fC*fA*fU*fG*fG | XXXXX XXXXX XXXXX |
| 31144 | | UUCUAAACAAUCCU | *fA*fA*fG*mG*mU*mU*mC*mU*mA*mA | XXXXX XXXXX XXXX |
| | | | *mA*C*b001A*A*mU*mC*mC*mU | |
| WV- | 442 | UCUCUUUCCAUGGAAG | fU*fC*fU*fC*fU*fU*fU*fC*fC*fA*fU*fG | XXXXX XXXXX XXXXX |
| 31632 | | GUUCUAAACCAUCCUG | *fG*fA*fA*fG*mG*mU*mU*mC*mU*mA* | XXXXX XXXXX XXXXX |
| | | | mA*mA*C*b002C*A*mU*mC*mC*mU*mG | X |
| WV- | 443 | UCUCUUUCCAUGGAAG | fU*fC*fU*fC*fU*fU*fU*fC*fC*fA*fU*fG | XXXXX XXXXX XXXXX |
| 31633 | | GUUCUAAACCAUCCUG | *fG*fA*fA*fG*mG*mU*mU*mC*mU*mA* | XXXXX XXXXX XXXXX |
| | | | mA*mA*C*b003C*A*mU*mC*mC*mU*mG | X |
| WV- | 444 | CUCUUUCCAUGGAAGG | fC*fU*fC*fU*fU*fU*fC*fC*fA*fU*fG*fG | XXXXX XXXXX XXXXX |
| 31634 | | UUCUAAACCAUCCU | *fA*fA*fG*mG*mU*mU*mC*mU*mA*mA | XXXXX XXXXX XXXX |
| | | | *mA*C*b002C*A*mU*mC*mC*mU | |
| WV- | 445 | CUCUUUCCAUGGAAGG | fC*fU*fC*fU*fU*fU*fC*fC*fA*fU*fG*fG | XXXXX XXXXX XXXXX |
| 31635 | | UUCUAAACCAUCCU | *fA*fA*fG*mG*mU*mU*mC*mU*mA*mA | XXXXX XXXXX XXXX |
| | | | *mA*C*b003C*A*mU*mC*mC*mU | |
| WV- | 446 | UCUCUUUCCAUGGAAG | fU*fC*fU*fC*fU*fU*fU*fC*fC*fA*fU*fG | XXXXX XXXXX XXXXX |
| 31748 | | GUUCUAAACCAUCCUG | *fG*fA*fA*fG*mG*mU*mU*mC*mU*mA* | XXXXX XXXXX XXXXX |
| | | | mA*mA*C*aC*A*mU*mC*mC*mU*mG | X |
| WV- | 447 | CUCUUUCCAUGGAAGG | fC*fU*fC*fU*fU*fU*fC*fC*fA*fU*fG*fG | XXXXX XXXXX XXXXX |
| 31749 | | UUCUAAACCAUCCU | *fA*fA*fG*mG*mU*mU*mC*mU*mA*mA | XXXXX XXXXX XXXX |
| | | | *mA*C*aC*A*mU*mC*mC*mU | |
| WV- | 448 | CUCUUUCCAUGGAAGG | mC*mU*mC*mU*fU*fU*fC*fC*fA*fU*fG*fG*fA*fA* | XXXXXXXXXXXXXXXXX |
| 32713 | | UUCUAAACCAUCCU | fG*mG*mU*mU*mC*mU*mA*mA*mA*C*C*A*mU*m | XXXXXXXXXXXX |
| | | | C*mC*mU | |
| WV- | 449 | CUCUUUCCAUGGAAGG | mC*mU*mC*mU*fU*fU*fC*fC*fA*fU*fG*fG*fA*fA* | XXXXXXXXXXXXXXXXX |
| 32714 | | UUCUAAACCAUCCU | fG*fG*mU*mU*mC*mU*mA*mA*mA*C*C*A*mU*mC | XXXXXXXXXXXX |
| | | | *mC*mU | |
| WV- | 450 | CUCUUUCCAUGGAAGG | mC*mU*mC*mU*fU*fU*fC*fC*fA*fU*fG*fG*fA*fA* | XXXXXXXXXXXXXXXXX |
| 32715 | | UUCUAAACCAUCCU | fG*mG*fU*mU*mC*mU*mA*mA*mA*C*C*A*mU*mC | XXXXXXXXXXXX |
| | | | *mC*mU | |
| WV- | 451 | CUCUUUCCAUGGAAGG | mfC*mU*mC*mU*fU*fU*fC*fC*fA*fU*fG*fG*fA*fA* | XXXXXXXXXXXXXXXXX |
| 32716 | | UUCUAAACCAUCCU | G*mG*mU*fU*mC*mU*mA*mA*mA*C*C*A*mU*mC | XXXXXXXXXXXX |
| | | | *mC*mU | |
| WV- | 452 | CUCUUUCCAUGGAAGG | mC*mU*mC*mU*fU*fU*fC*fC*fA*fU*fG*fG*fA*fA* | XXXXXXXXXXXXXXXXX |
| 32717 | | UUCUAAACCAUCCU | fG*mG*mU*mU*fC*mU*mA*mA*mA*C*C*A*mU*mC | XXXXXXXXXXXX |
| | | | *mC*mU | |
| WV- | 453 | CUCUUUCCAUGGAAGG | mC*mU*mC*mU*fU*fU*fC*fC*fA*fU*fG*fG*fA*fA* | XXXXXXXXXXXXXXXXX |
| 32718 | | UUCUAAACCAUCCU | fG*mG*mU*mU*mC*fU*mA*mA*mA*C*C*A*mU*mC | XXXXXXXXXXXX |
| | | | *mC*mU | |
| WV- | 454 | CUCUUUCCAUGGAAGG | mC*mU*mC*mU*fU*fU*fC*fC*fA*fU*fG*fG*fA*fA* | XXXXXXXXXXXXXXXXX |
| 32719 | | UUCUAAACCAUCCU | fG*mG*mU*mU*mC*mU*fA*mA*mA*C*C*A*mU*mC | XXXXXXXXXXXX |
| | | | *mC*mU | |
| WV- | 455 | CUCUUUCCAUGGAAGG | mC*mU*mC*mU*fU*fU*fC*fC*fA*fU*fG*fG*fA*fA*f | XXXXXXXXXXXXXXXXX |
| 32720 | | UUCUAAACCAUCCU | G*mG*mU*mU*mC*mU*mA*fA*mA*C*C*A*mU*mC | XXXXXXXXXXXX |
| | | | *mC*mU | |
| WV- | 456 | CUCUUUCCAUGGAAGG | mC*mU*mC*mU*fU*fU*fC*fC*fA*fU*fG*fG*fA*fA*f | XXXXXXXXXXXXXXXXX |
| 32721 | | UUCUAAACCAUCCU | G*mG*mU*mU*mC*mU*mA*mA*fA*C*C*A*mU*mC | XXXXXXXXXXXX |
| | | | *mC*mU | |
| WV- | 457 | CUCUUUCCAUGGAAGG | mC*mU*mC*mU*fU*fU*fC*fC*fA*fU*fG*fG*fA*fA*f | XXXXXXXXXXXXXXXXX |
| 32722 | | UUCUAAACCAUCCU | G*mG*mU*mU*mC*mU*mA*mA*mA*C*C*A*fU*mC | XXXXXXXXXXXX |
| | | | *mC*mU | |
| WV- | 458 | CUCUUUCCAUGGAAGG | mC*mU*mC*mU*fU*fU*fC*fC*fA*fU*fG*fG*fA*fA*f | XXXXXXXXXXXXXXXXX |
| 32723 | | UUCUAAACCAUCCU | G*mG*mU*mU*mC*mU*mA*mA*mA*C*C*A*mU*fC | XXXXXXXXXXXX |
| | | | *mC*mU | |
| WV- | 459 | CUCUUUCCAUGGAAGG | mC*mU*mC*mU*fU*fU*fC*fC*fA*fU*fG*fG*fA*fA*f | XXXXXXXXXXXXXXXXX |
| 32724 | | UUCUAAACCAUCCU | G*mG*mU*mU*mC*mU*mA*mA*mA*C*C*A*mU*m | XXXXXXXXXXXX |
| | | | C*fC*mU | |
| WV- | 460 | CUCUUUCCAUGGAAGG | mC*mU*mC*mU*fU*fU*fC*fC*fA*fU*fG*fG*fA*fA*f | XXXXXXXXXXXXXXXXX |
| 32725 | | UUCUAAACCAUCCU | G*mG*mU*mU*mC*mU*mA*mA*mA*C*C*A*mU*m | XXXXXXXXXXXX |
| | | | C*mC*fU | |
| WV- | 461 | CUCUUUCCAUGGAAGG | fC*fU*fC*fU*fU*fU*fC*fC*Aeo*fU*fG*fG*fA*fA*fG* | XXXXXXXXXXXXXXXXX |
| 32734 | | UUCUAAACCAUCCU | mG*mU*mU*mC*mU*mA*mA*mA*C*C*A*mU*mC* | XXXXXXXXXXXX |
| | | | mC*mU | |
| WV- | 462 | CUCUUUCCAUGGAAGG | fC*fU*fC*fU*fU*fU*fC*fC*fA*fU*Geo*fG*fA*fA*fG* | XXXXXXXXXXXXXXXXX |
| 32736 | | UUCUAAACCAUCCU | mG*mU*mU*mC*mU*mA*mA*mA*C*C*A*mU*mC* | XXXXXXXXXXXX |
| | | | mC*mU | |
| WV- | 463 | CUCUUUCCAUGGAAGG | fC*fU*fC*fU*fU*fU*fC*fC*fA*fU*fG*Geo*fA*fA*fG* | XXXXXXXXXXXXXXXXX |
| 32737 | | UUCUAAACCAUCCU | mG*mU*mU*mC*mU*mA*mA*mA*C*C*A*mU*mC* | XXXXXXXXXXXX |
| | | | mC*mU | |
| WV- | 464 | CUCUUUCCAUGGAAGG | fC*fU*fC*fU*fU*fU*fC*fC*fA*fU*fG*fG*Aeo*fA*fG* | XXXXXXXXXXXXXXXXX |
| 32738 | | UUCUAAACCAUCCU | mG*mU*mU*mC*mU*mA*mA*mA*C*C*A*mU*mC* | XXXXXXXXXXXX |
| | | | mC*mU | |
| WV- | 465 | CUCUUUCCAUGGAAGG | fC*fU*fC*fU*fU*fU*fC*fC*fA*fU*fG*fG*fA*Aeo*fG* | XXXXXXXXXXXXXXXXX |
| 32739 | | UUCUAAACCAUCCU | mG*mU*mU*mC*mU*mA*mA*mA*C*C*A*mU*mC* | XXXXXXXXXXXX |
| | | | mC*mU | |
| WV- | 466 | CUCUUUCCAUGGAAGG | fC*fU*fC*fU*fU*fU*fC*fC*fA*fU*fG*fG*fA*fA*Geo* | XXXXXXXXXXXXXXXXX |
| 32740 | | UUCUAAACCAUCCU | mG*mU*mU*mC*mU*mA*mA*mA*C*C*A*mU*mC* | XXXXXXXXXXXX |
| | | | mC*mU | |
| WV- | 467 | CUCUUUCCAUGGAAGG | fC*fU*fC*fU*fU*fU*fC*fC*fA*fU*fG*fG*fA*fA*fG*G | XXXXXXXXXXXXXXXXX |
| 32741 | | UUCUAAACCAUCCU | eo*mU*mU*mC*mU*mA*mA*mA*C*C*A*mU*mC*m | XXXXXXXXXXXX |
| | | | C*mU | |
| WV- | 468 | CUCUUUCCAUGGAAGG | fC*fU*fC*fU*fU*fU*fC*fC*fA*fU*fG*fG*fA*fA*fG*m | XXXXXXXXXXXXXXXXX |
| 32746 | | UUCUAAACCAUCCU | G*mU*mU*mC*mU*Aeo*mA*mA*C*C*A*mU*mC*m | XXXXXXXXXXXX |
| | | | C*mU | |
| WV- | 469 | CUCUUUCCAUGGAAGG | fC*fU*fC*fU*fU*fU*fC*fC*fA*fU*fG*fG*fA*fA*fG*m | XXXXXXXXXXXXXXXXX |
| 32747 | | UUCUAAACCAUCCU | G*mU*mU*mC*mU*mA*Aeo*mA*C*C*A*mU*mC*m | XXXXXXXXXXXX |
| | | | C*mU | |
| WV- | 470 | CUCUUUCCAUGGAAGG | fC*fU*fC*fU*fU*fU*fC*fC*fA*fU*fG*fG*fA*fA*fG*m | XXXXXXXXXXXXXXXXX |
| 32748 | | UUCUAAACCAUCCU | G*mU*mU*mC*mU*mA*mA*Aeo*C*C*A*mU*mC*m | XXXXXXXXXXXX |
| | | | C*mU | |
| WV- | 471 | CUCUUUCCAUGGAAGG | fC*fU*fC*fU*fU*fU*fC*fC*fA*fU*fG*fG*fA*fA*fG*m | XXXXXXXXXXXXXXXXX |
| 32751 | | UUCUAAACCAUCCU | G*mU*mU*mC*mU*mA*mA*mA*C*C*Aeo*mU*mC* | XXXXXXXXXXXX |
| | | | mC*mU | |
| WV- | 472 | CUCUUUCCAUGGAAGG | fC*fU*fC*fU*fU*fU*fC*fC*fA*fU*fG*fG*fA*fA*fG*m | XXXXXXXXXXXXXXXXX |
| 32756 | | UUCUAAACCAUCCU | G*mU*mU*mC*mU*mA*mA*mAC*C*AmU*mC*mC* | XXXXXOXXOXXX |
| | | | mU | |
| WV- | 473 | CUCUUUCCAUGGAAGG | fCfU*fC*fU*fU*fU*fC*fC*fA*fU*fG*fG*fA*fA*fG*m | OXXXXXXXXXXXXXXXX |
| 32757 | | UUCUAAACCAUCCU | G*mU*mU*mC*mU*mA*mA*mA*C*C*A*mU*mC*m | XXXXXXXXXXXX |
| | | | C*mU | |
| WV- | 474 | CUCUUUCCAUGGAAGG | fC*fUfC*fU*fU*fU*fC*fC*fA*fU*fG*fG*fA*fA*fG*m | XOXXXXXXXXXXXXXXX |
| 32758 | | UUCUAAACCAUCCU | G*mU*mU*mC*mU*mA*mA*mA*C*C*A*mU*mC*m | XXXXXXXXXXXX |
| | | | C*mU | |
| WV- | 475 | CUCUUUCCAUGGAAGG | fC*fU*fCfU*fU*fU*fC*fC*fA*fU*fG*fG*fA*fA*fG*m | XXOXXXXXXXXXXXXXX |
| 32759 | | UUCUAAACCAUCCU | G*mU*mU*mC*mU*mA*mA*mA*C*C*A*mU*mC*m | XXXXXXXXXXXX |
| | | | C*mU | |
| WV- | 476 | CUCUUUCCAUGGAAGG | fC*fU*fC*fUfU*fU*fC*fC*fA*fU*fG*fG*fA*fA*fG*m | XXXOXXXXXXXXXXXXX |
| 32760 | | UUCUAAACCAUCCU | G*mU*mU*mC*mU*mA*mA*mA*C*C*A*mU*mC*m | XXXXXXXXXXXX |
| | | | C*mU | |
| WV- | 477 | CUCUUUCCAUGGAAGG | fC*fU*fC*fU*fUfU*fC*fC*fA*fU*fG*fG*fA*fA*fG*m | XXXXOXXXXXXXXXXXX |
| 32761 | | UUCUAAACCAUCCU | G*mU*mU*mC*mU*mA*mA*mA*C*C*A*mU*mC*m | XXXXXXXXXXXX |
| | | | C*mU | |
| WV- | 478 | CUCUUUCCAUGGAAGG | fC*fU*fC*fU*fU*fUfC*fC*fA*fU*fG*fG*fA*fA*fG*m | XXXXXOXXXXXXXXXXX |
| 32762 | | UUCUAAACCAUCCU | G*mU*mU*mC*mU*mA*mA*mA*C*C*A*mU*mC*m | XXXXXXXXXXXX |
| | | | C*mU | |
| WV- | 479 | CUCUUUCCAUGGAAGG | fC*fU*fC*fU*fU*fU*fCfC*fA*fU*fG*fG*fA*fA*fG*m | XXXXXXOXXXXXXXXXX |
| 32763 | | UUCUAAACCAUCCU | G*mU*mU*mC*mU*mA*mA*mA*C*C*A*mU*mC*m | XXXXXXXXXXXX |
| | | | C*mU | |
| WV- | 480 | CUCUUUCCAUGGAAGG | fC*fU*fC*fU*fU*fU*fC*fCfA*fU*fG*fG*fA*fA*fG*m | XXXXXXXOXXXXXXXXX |
| 32764 | | UUCUAAACCAUCCU | G*mU*mU*mC*mU*mA*mA*mA*C*C*A*mU*mC*m | XXXXXXXXXXXX |
| | | | C*mU | |
| WV- | 481 | CUCUUUCCAUGGAAGG | fC*fU*fC*fU*fU*fU*fC*fC*fAfU*fG*fG*fA*fA*fG*m | XXXXXXXXOXXXXXXXX |
| 32765 | | UUCUAAACCAUCCU | G*mU*mU*mC*mU*mA*mA*mA*C*C*A*mU*mC*m | XXXXXXXXXXXX |
| | | | C*mU | |
| WV- | 482 | CUCUUUCCAUGGAAGG | fC*fU*fC*fU*fU*fU*fC*fC*fA*fUfG*fG*fA*fA*fG*m | XXXXXXXXXOXXXXXXX |
| 32766 | | UUCUAAACCAUCCU | G*mU*mU*mC*mU*mA*mA*mA*C*C*A*mU*mC*m | XXXXXXXXXXXX |
| | | | C*mU | |
| WV- | 483 | CUCUUUCCAUGGAAGG | fC*fU*fC*fU*fU*fU*fC*fC*fA*fU*fGfG*fA*fA*fG*m | XXXXXXXXXXOXXXXXX |
| 32767 | | UUCUAAACCAUCCU | G*mU*mU*mC*mU*mA*mA*mA*C*C*A*mU*mC*m | XXXXXXXXXXXX |
| | | | C*mU | |
| WV- | 484 | CUCUUUCCAUGGAAGG | fC*fU*fC*fU*fU*fU*fC*fC*fA*fU*fG*fGfA*fA*fG*m | XXXXXXXXXXXOXXXXX |
| 32768 | | UUCUAAACCAUCCU | G*mU*mU*mC*mU*mA*mA*mA*C*C*A*mU*mC*m | XXXXXXXXXXXX |
| | | | C*mU | |
| WV- | 485 | CUCUUUCCAUGGAAGG | fC*fU*fC*fU*fU*fU*fC*fC*fA*fU*fG*fG*fAfA*fG*m | XXXXXXXXXXXXOXXXX |
| 32769 | | UUCUAAACCAUCCU | G*mU*mU*mC*mU*mA*mA*mA*C*C*A*mU*mC*m | XXXXXXXXXXXX |
| | | | C*mU | |
| WV- | 486 | CUCUUUCCAUGGAAGG | fC*fU*fC*fU*fU*fU*fC*fC*fA*fU*fG*fG*fA*fAfG*m | XXXXXXXXXXXXXOXXX |
| 32770 | | UUCUAAACCAUCCU | G*mU*mU*mC*mU*mA*mA*mA*C*C*A*mU*mC*m | XXXXXXXXXXXX |
| | | | C*mU | |
| WV- | 487 | CUCUUUCCAUGGAAGG | fC*fU*fC*fU*fU*fU*fC*fC*fA*fU*fG*fG*fA*fA*fGm | XXXXXXXXXXXXXXOXX |
| 32771 | | UUCUAAACCAUCCU | G*mU*mU*mC*mU*mA*mA*mA*C*C*A*mU*mC*m | XXXXXXXXXXXX |
| | | | C*mU | |
| WV- | 488 | CUCUUUCCAUGGAAGG | fC*fU*fC*fU*fU*fU*fC*fC*fA*fU*fG*fG*fA*fA*fG*m | XXXXXXXXXXXXXXXOX |
| 32772 | | UUCUAAACCAUCCU | GmU*mU*mC*mU*mA*mA*mA*C*C*A*mU*mC*mC | XXXXXXXXXXXX |
| | | | *mU | |
| WV- | 489 | CUCUUUCCAUGGAAGG | fC*fU*fC*fU*fU*fU*fC*fC*fA*fU*fG*fG*fA*fA*fG*m | XXXXXXXXXXXXXXXXO |
| 32773 | | UUCUAAACCAUCCU | G*mUmU*mC*mU*mA*mA*mA*C*C*A*mU*mC*mC | XXXXXXXXXXXX |
| | | | *mU | |
| WV- | 490 | CUCUUUCCAUGGAAGG | fC*fU*fC*fU*fU*fU*fC*fC*fA*fU*fG*fG*fA*fA*fG*m | XXXXXXXXXXXXXXXXX |
| 32774 | | UUCUAAACCAUCCU | G*mU*mUmC*mU*mA*mA*mA*C*C*A*mU*mC*mC | OXXXXXXXXXXX |
| | | | *mU | |
| WV- | 491 | CUCUUUCCAUGGAAGG | fC*fU*fC*fU*fU*fU*fC*fC*fA*fU*fG*fG*fA*fA*fG*m | XXXXXXXXXXXXXXXXX |
| 32775 | | UUCUAAACCAUCCU | G*mU*mU*mCmU*mA*mA*mA*C*C*A*mU*mC*mC | XOXXXXXXXXXX |
| | | | *mU | |
| WV- | 492 | CUCUUUCCAUGGAAGG | fC*fU*fC*fU*fU*fU*fC*fC*fA*fU*fG*fG*fA*fA*fG*m | XXXXXXXXXXXXXXXXX |
| 32776 | | UUCUAAACCAUCCU | G*mU*mU*mC*mUmA*mA*mA*C*C*A*mU*mC*mC | XXOXXXXXXXXX |
| | | | *mU | |
| WV- | 493 | CUCUUUCCAUGGAAGG | fC*fU*fC*fU*fU*fU*fC*fC*fA*fU*fG*fG*fA*fA*fG*m | XXXXXXXXXXXXXXXXX |
| 32777 | | UUCUAAACCAUCCU | G*mU*mU*mC*mU*mAmA*mA*C*C*A*mU*mC*mC | XXXOXXXXXXXX |
| | | | *mU | |
| WV- | 494 | CUCUUUCCAUGGAAGG | fC*fU*fC*fU*fU*fU*fC*fC*fA*fU*fG*fG*fA*fA*fG*m | XXXXXXXXXXXXXXXXX |
| 32778 | | UUCUAAACCAUCCU | G*mU*mU*mC*mU*mA*mAmA*C*C*A*mU*mC*mC | XXXXOXXXXXXX |
| | | | *mU | |
| WV- | 495 | CUCUUUCCAUGGAAGG | fC*fU*fC*fU*fU*fU*fC*fC*fA*fU*fG*fG*fA*fA*fG*m | XXXXXXXXXXXXXXXXX |
| 32779 | | UUCUAAACCAUCCU | G*mU*mU*mC*mU*mA*mA*mAC*C*A*mU*mC*mC | XXXXXOXXXXXX |
| | | | *mU | |
| WV- | 496 | CUCUUUCCAUGGAAGG | fC*fU*fC*fU*fU*fU*fC*fC*fA*fU*fG*fG*fA*fA*fG*m | XXXXXXXXXXXXXXXXX |
| 32780 | | UUCUAAACCAUCCU | G*mU*mU*mC*mU*mA*mA*mA*CC*A*mU*mC*mC | XXXXXXOXXXXX |
| | | | *mU | |
| WV- | 497 | CUCUUUCCAUGGAAGG | fC*fU*fC*fU*fU*fU*fC*fC*fA*fU*fG*fG*fA*fA*fG*m | XXXXXXXXXXXXXXXXX |
| 32781 | | UUCUAAACCAUCCU | G*mU*mU*mC*mU*mA*mA*mA*C*CA*mU*mC*mC | XXXXXXXOXXXX |
| | | | *mU | |
| WV- | 498 | CUCUUUCCAUGGAAGG | fC*fU*fC*fU*fU*fU*fC*fC*fA*fU*fG*fG*fA*fA*fG*m | XXXXXXXXXXXXXXXXX |
| 32782 | | UUCUAAACCAUCCU | G*mU*mU*mC*mU*mA*mA*mA*C*C*AmU*mC*mC | XXXXXXXXOXXX |
| | | | *mU | |
| WV- | 499 | CUCUUUCCAUGGAAGG | fC*fU*fC*fU*fU*fU*fC*fC*fA*fU*fG*fG*fA*fA*fG*m | XXXXXXXXXXXXXXXXX |
| 32783 | | UUCUAAACCAUCCU | G*mU*mU*mC*mU*mA*mA*mA*C*C*A*mUmC*mC | XXXXXXXXXOXX |
| | | | *mU | |
| WV- | 500 | CUCUUUCCAUGGAAGG | fC*fU*fC*fU*fU*fU*fC*fC*fA*fU*fG*fG*fA*fA*fG*m | XXXXXXXXXXXXXXXXX |
| 32784 | | UUCUAAACCAUCCU | G*mU*mU*mC*mU*mA*mA*mA*C*C*A*mU*mCmC | XXXXXXXXXXOX |
| | | | *mU | |
| WV- | 501 | CUCUUUCCAUGGAAGG | fC*fU*fC*fU*fU*fU*fC*fC*fA*fU*fG*fG*fA*fA*fG*m | XXXXXXXXXXXXXXXXX |
| 32785 | | UUCUAAACCAUCCU | G*mU*mU*mC*mU*mA*mA*mA*C*C*A*mU*mC*m | XXXXXXXXXXXO |
| | | | CmU | |
| WV- | 502 | CUCUUUCCAUGGAAGG | mC*mU*mC*mU*fU*fU*fC*fC*fA*fU*fG*fG*fA*fA*f | XXXXXXXXXXXXXXXXX |
| 32786 | | UUCUAAACCAUCCU | G*mG*mU*mU*mC*mU*mA*mA*mA*C*C*A*mU*fC | XXXXXXXXXXXX |
| | | | *fC*fU | |
| WV- | 503 | CUCUUUCCAUGGAAGG | fC*fU*fC*fU*fU*fU*fC*fC*fA*fU*fG*fG*fA*fA*fG*m | XXXXXXXXXXXXXXXXX |
| 32790 | | UUCUAAACCAUCCU | G*mU*mU*mC*mU*mA*mA*mA*C*C*A*mU*fC*fC* | XXXXXXXXXXXX |
| | | | fU | |
| WV- | 504 | CTCUUUCCAUGGAAGG | fC*Teo*fC*fU*fU*fU*fC*fC*fA*fU*fG*fG*fA*fA*fG* | XXXXXXXXXXXXXXXXX |
| 33185 | | UUCUAAACCAUCCU | mG*mU*mU*mC*mU*mA*mA*mA*C*C*A*mU*mC* | XXXXXXXXXXXX |
| | | | mC*mU | |
| WV- | 505 | CUCTUUCCAUGGAAGG | fC*fU*fC*Teo*fU*fU*fC*fC*fA*fU*fG*fG*fA*fA*fG* | XXXXXXXXXXXXXXXXX |
| 33186 | | UUCUAAACCAUCCU | mG*mU*mU*mC*mU*mA*mA*mA*C*C*A*mU*mC* | XXXXXXXXXXXX |
| | | | mC*mU | |
| WV- | 506 | CUCUTUCCAUGGAAGG | fC*fU*fC*fU*Teo*fU*fC*fC*fA*fU*fG*fG*fA*fA*fG* | XXXXXXXXXXXXXXXXX |
| 33187 | | UUCUAAACCAUCCU | mG*mU*mU*mC*mU*mA*mA*mA*C*C*A*mU*mC* | XXXXXXXXXXXX |
| | | | mC*mU | |
| WV- | 507 | CUCUUTCCAUGGAAGG | fC*fU*fC*fU*fU*Teo*fC*fC*fA*fU*fG*fG*fA*fA*fG* | XXXXXXXXXXXXXXXXX |
| 33188 | | UUCUAAACCAUCCU | mG*mU*mU*mC*mU*mA*mA*mA*C*C*A*mU*mC* | XXXXXXXXXXXX |
| | | | mC*mU | |
| WV- | 508 | CUCUUUCCATGGAAGG | fC*fU*fC*fU*fU*fU*fC*fC*fA*Teo*fG*fG*fA*fA*fG* | XXXXXXXXXXXXXXXXX |
| 33189 | | UUCUAAACCAUCCU | mG*mU*mU*mC*mU*mA*mA*mA*C*C*A*mU*mC* | XXXXXXXXXXXX |
| | | | mC*mU | |
| WV- | 509 | CUCUUUCCAUGGAAGG | fC*fU*fC*fU*fU*fU*fC*fC*fA*fU*fG*fG*fA*fA*fG*m | XXXXXXXXXXXXXXXXX |
| 33190 | | TUCUAAACCAUCCU | G*Teo*mU*mC*mU*mA*mA*mA*C*C*A*mU*mC*m | XXXXXXXXXXXX |
| | | | C*mU | |
| WV- | 510 | CUCUUUCCAUGGAAGG | fC*fU*fC*fU*fU*fU*fC*fC*fA*fU*fG*fG*fA*fA*fG*m | XXXXXXXXXXXXXXXXX |
| 33191 | | UTCUAAACCAUCCU | G*mU*Teo*mC*mU*mA*mA*mA*C*C*A*mU*mC*m | XXXXXXXXXXXX |
| | | | C*mU | |
| WV- | 511 | CUCUUUCCAUGGAAGG | fC*fU*fC*fU*fU*fU*fC*fC*fA*fU*fG*fG*fA*fA*fG*m | XXXXXXXXXXXXXXXXX |
| 33192 | | UUCTAAACCAUCCU | G*mU*mU*mC*Teo*mA*mA*mA*C*C*A*mU*mC*m | XXXXXXXXXXXX |
| | | | C*mU | |
| WV- | 512 | CUCUUUCCAUGGAAGG | fC*fU*fC*fU*fU*fU*fC*fC*fA*fU*fG*fG*fA*fA*fG*m | XXXXXXXXXXXXXXXXX |
| 33193 | | UUCUAAACCATCCU | G*mU*mU*mC*mU*mA*mA*mA*C*C*A*Teo*mC*m | XXXXXXXXXXXX |
| | | | C*mU | |
| WV- | 513 | CUCUUUCCAUGGAAGG | fC*fU*fC*fU*fU*fU*fC*fC*fA*fU*fG*fG*fA*fA*fG*m | XXXXXXXXXXXXXXXXX |
| 33194 | | UUCUAAACCAUCCT | G*mU*mU*mC*mU*mA*mA*mA*C*C*A*mU*mC*m | XXXXXXXXXXXX |
| | | | C+Teo | |
| WV- | 514 | CUCUUUCCAUGGAAGG | m5Ceo*fU*fC*fU*fU*fU*fC*fC*fA*fU*fG*fG*fA*fA*f | XXXXXXXXXXXXXXXXX |
| 33851 | | UUCUAAACCAUCCU | G*mG*mU*mU*mC*mU*mA*mA*mA*C*C*A*mU*m | XXXXXXXXXXXX |
| | | | C*mC*mU | |
| WV- | 515 | CUCUUUCCAUGGAAGG | fC*fU*m5Ceo*fU*fU*fU*fC*fC*fA*fU*fG*fG*fA*fA*f | XXXXXXXXXXXXXXXXX |
| 33852 | | UUCUAAACCAUCCU | G*mG*mU*mU*mC*mU*mA*mA*mA*C*C*A*mU*m | XXXXXXXXXXXX |
| | | | C*mC*mU | |
| WV- | 516 | CUCUUUCCAUGGAAGG | fC*fU*fC*fU*fU*fU*m5Ceo*fC*fA*fU*fG*fG*fA*fA*f | XXXXXXXXXXXXXXXXX |
| 33853 | | UUCUAAACCAUCCU | G*mG*mU*mU*mC*mU*mA*mA*mA*C*C*A*mU*m | XXXXXXXXXXXX |
| | | | C*mC*mU | |
| WV- | 517 | CUCUUUCCAUGGAAGG | fC*fU*fC*fU*fU*fU*fC*m5Ceo*fA*fU*fG*fG*fA*fA*f | XXXXXXXXXXXXXXXXX |
| 33854 | | UUCUAAACCAUCCU | G*mG*mU*mU*mC*mU*mA*mA*mA*C*C*A*mU*m | XXXXXXXXXXXX |
| | | | C*mC*mU | |
| WV- | 518 | CUCUUUCCAUGGAAGG | fC*fU*fC*fU*fU*fU*fC*fC*fA*fU*fG*fG*fA*fA*fG*m | XXXXXXXXXXXXXXXXX |
| 33855 | | UUCUAAACCAUCCU | G*mU*mU*m5Ceo*mU*mA*mA*mA*C*C*A*mU*mC | XXXXXXXXXXXX |
| | | | *mC*mU | |
| WV- | 519 | CUCUUUCCAUGGAAGG | fC*fU*fC*fU*fU*fU*fC*fC*fA*fU*fG*fG*fA*fA*fG*m | XXXXXXXXXXXXXXXXX |
| 33856 | | UUCUAAACCAUCCU | G*mU*mU*mC*mU*mA*mA*mA*m5Ceo*C*A*mU*m | XXXXXXXXXXXX |
| | | | C*mC*mU | |
| WV- | 520 | CUCUUUCCAUGGAAGG | fC*fU*fC*fU*fU*fU*fC*fC*fA*fU*fG*fG*fA*fA*fG*m | XXXXXXXXXXXXXXXXX |
| 33857 | | UUCUAAACCAUCCU | G*mU*mU*mC*mU*mA*mA*mA*C*m5Ceo*A*mU*m | XXXXXXXXXXXX |
| | | | C*mC*mU | |
| WV- | 521 | CUCUUUCCAUGGAAGG | fC*fU*fC*fU*fU*fU*fC*fC*fA*fU*fG*fG*fA*fA*fG*m | XXXXXXXXXXXXXXXXX |
| 33858 | | UUCUAAACCAUCCU | G*mU*mU*mC*mU*mA*mA*mA*C*C*A*mU*m5Ceo | XXXXXXXXXXXX |
| | | | *mC*mU | |
| WV- | 522 | CUCUUUCCAUGGAAGG | fC*fU*fC*fU*fU*fU*fC*fC*fA*fU*fG*fG*fA*fA*fG*m | XXXXXXXXXXXXXXXXX |
| 33859 | | UUCUAAACCAUCCU | G*mU*mU*mC*mU*mA*mA*mA*C*C*A*mU*mC*m | XXXXXXXXXXXX |
| | | | 5Ceo*mU | |
| WV- | 523 | CTCTUUCCAUGGAAGG | m5Ceo*Teo*m5Ceo*Teo*fU*fU*fC*fC*fA*fU*fG*fG*f | XXXXXXXXXXXXXXXXX |
| 33861 | | UUCUAAACCAUCCU | A*fA*fG*mG*mU*mU*mC*mU*mA*mA*mA*C*C*A* | XXXXXXXXXXXX |
| | | | mU*mC*mC*mU | |
| WV- | 524 | CTCTUUCCAUGGAAGG | m5Ceo*Teo*m5Ceo*Teo*fU*fU*fC*fC*fA*fU*fG*fG*f | XXXXXXXXXXXXXXXXX |
| 33862 | | UUCUAAACCAUCCU | A*fA*fG*mG*mU*mU*mC*mU*mA*mA*mA*C*C*A* | XXXXXXXXXXXX |
| | | | mU*fC*fC*fU | |
| WV- | 525 | CUCUUUCCAUGGAAGG | mC*fU*fC*fU*fU*fU*fC*fC*fA*fU*fG*fG*fA*fA*fG* | XXXXXXXXXXXXXXXXX |
| 36884 | | UUCUAAACCAUCCU | mG*mU*mU*mC*mU*mA*mA*mA*C*C*A*mU*mC* | XXXXXXXXXXXX |
| | | | mC*mU | |
| WV- | 526 | CUCUUUCCAUGGAAGG | mC*SfU*fC*fU*fU*fU*fC*fC*fA*fU*fG*fG*fA*fA*fG | SXXXXXXXXXXXXXXXX |
| 36885 | | UUCUAAACCAUCCU | *mG*mU*mU*mC*mU*mA*mA*mA*C*C*A*mU*mC | XXXXXXXXXXXX |
| | | | *mC*mU | |
| WV- | 527 | CUCUUUCCAUGGAAGG | mC*SfU*SfC*fU*fU*fU*fC*fC*fA*fU*fG*fG*fA*fA*f | SSXXXXXXXXXXXXXXXX |
| 36886 | | UUCUAAACCAUCCU | G*mG*mU*mU*mC*mU*mA*mA*mA*C*C*A*mU*m | XXXXXXXXXXX |
| | | | C*mC*mU | |
| WV- | 528 | CUCUUUCCAUGGAAGG | mC*SfU*SfC*SfU*fU*fU*fC*fC*fA*fU*fG*fG*fA*fA* | SSSXXXXXXXXXXXXXXX |
| 36887 | | UUCUAAACCAUCCU | fG*mG*mU*mU*mC*mU*mA*mA*mA*C*C*A*mU*m | XXXXXXXXXXX |
| | | | C*mC*mU | |
| WV- | 529 | CUCUUUCCAUGGAAGG | mC*SfU*SfC*SfU*SfU*fU*fC*fC*fA*fU*fG*fG*fA*fA | SSSSXXXXXXXXXXXXXX |
| 36888 | | UUCUAAACCAUCCU | *fG*mG*mU*mU*mC*mU*mA*mA*mA*C*C*A*mU* | XXXXXXXXXXX |
| | | | mC*mC*mU | |
| WV- | 530 | CUCUUUCCAUGGAAGG | mC*SfU*SfC*SfU*SfU*SfU*fC*fC*fA*fU*fG*fG*fA*f | SSSSSXXXXXXXXXXXXX |
| 36889 | | UUCUAAACCAUCCU | A*fG*mG*mU*mU*mC*mU*mA*mA*mA*C*C*A*mU | XXXXXXXXXXX |
| | | | *mC*mC*mU | |
| WV- | 531 | CUCUUUCCAUGGAAGG | mC*SfU*SfC*SfU*SfU*SfU*SfC*fC*fA*fU*fG*fG*fA* | SSSSSSXXXXXXXXXXXXX |
| 36890 | | UUCUAAACCAUCCU | fA*fG*mG*mU*mU*mC*mU*mA*mA*mA*C*C*A*m | XXXXXXXXXX |
| | | | U*mC*mC*mU | |
| WV- | 532 | CUCUUUCCAUGGAAGG | ₘC*SfU*SfC*SfU*SfU*SfU*SfC*SfC*fA*fU*fG*fG*fA | SSSSSSSXXXXXXXXXXXX |
| 36891 | | UUCUAAACCAUCCU | *fA*fG*mG*mU*mU*mC*mU*mA*mA*mA*C*C*A*m | XXXXXXXXXX |
| | | | U*mC*mC*mU | |
| WV- | 533 | CUCUUUCCAUGGAAGG | mC*SfU*SfC*SfU*SfU*SfU*SfC*SfC*SfA*fU*fG*fG*f | SSSSSSSSXXXXXXXXXXX |
| 36892 | | UUCUAAACCAUCCU | A*fA*fG*mG*mU*mU*mC*mU*mA*mA*mA*C*C*A* | XXXXXXXXXX |
| | | | mU*mC*mC*mU | |
| WV- | 534 | CUCUUUCCAUGGAAGG | mC*SfU*SfC*SfU*SfU*SfU*SfC*SfC*SfA*SfU*fG*fG | SSSSSSSSSXXXXXXXXXX |
| 36893 | | UUCUAAACCAUCCU | *fA*fA*fG*mG*mU*mU*mC*mU*mA*mA*mA*C*C* | XXXXXXXXXX |
| | | | A*mU*mC*mC*mU | |
| WV- | 535 | CUCUUUCCAUGGAAGG | mC*SfU*SfC*SfU*SfU*SfU*SfC*SfC*SfA*SfU*SfG*f | SSSSSSSSSSXXXXXXXXX |
| 36894 | | UUCUAAACCAUCCU | G*fA*fA*fG*mG*mU*mU*mC*mU*mA*mA*mA*C*C | XXXXXXXXXX |
| | | | *A*mU*mC*mC*mU | |
| WV- | 536 | CUCUUUCCAUGGAAGG | mC*SfU*SfC*SfU*SfU*SfU*SfC*SfC*SfA*SfU*SfG*Sf | SSSSSSSSSSSXXXXXXXX |
| 36895 | | UUCUAAACCAUCCU | G*fA*fA*fG*mG*mU*mU*mC*mU*mA*mA*mA*C*C | XXXXXXXXXX |
| | | | *A*mU*mC*mC*mU | |
| WV- | 537 | CUCUUUCCAUGGAAGG | mC*SfU*SfC*SfU*SfU*SfU*SfC*SfC*SfA*SfU*SfG*Sf | SSSSSSSSSSSSXXXXXXX |
| 36896 | | UUCUAAACCAUCCU | G*SfA*fA*fG*mG*mU*mU*mC*mU*mA*mA*mA*C* | XXXXXXXXXX |
| | | | C*A*mU*mC*mC*mU | |
| WV- | 538 | CUCUUUCCAUGGAAGG | mC*SfU*SfC*SfU*SfU*SfU*SfC*SfC*SfA*SfU*SfG*Sf | SSSSSSSSSSSSSXXXXXX |
| 36897 | | UUCUAAACCAUCCU | G*SfA*SfA*fG*mG*mU*mU*mC*mU*mA*mA*mA*C | XXXXXXXXXX |
| | | | *C*A*mU*mC*mC*mU | |
| WV- | 539 | CUCUUUCCAUGGAAGG | mC*SfU*SfC*SfU*SfU*SfU*SfC*SfC*SfA*SfU*SfG*Sf | SSSSSSSSSSSSSSXXXXX |
| 36898 | | UUCUAAACCAUCCU | G*SfA*SfA*SfG*mG*mU*mU*mC*mU*mA*mA*mA* | XXXXXXXXXX |
| | | | C*C*A*mU*mC*mC*mU | |
| WV- | 540 | CUCUUUCCAUGGAAGG | mC*SfU*SfC*SfU*SfU*SfU*SfC*SfC*SfA*SfU*SfG*Sf | SSSSSSSSSSSSSSSXXXX |
| 36899 | | UUCUAAACCAUCCU | G*SfA*SfA*SfG*SmG*mU*mU*mC*mU*mA*mA*mA | XXXXXXXXXX |
| | | | *C*C*A*mU*mC*mC*mU | |
| WV- | 541 | CUCUUUCCAUGGAAGG | mC*SfU*SfC*SfU*SfU*SfU*SfC*SfC*SfA*SfU*SfG*Sf | SSSSSSSSSSSSSSSSXXX |
| 36900 | | UUCUAAACCAUCCU | G*SfA*SfA*SfG*SmG*SmU*mU*mC*mU*mA*mA*m | XXXXXXXXXX |
| | | | A*C*C*A*mU*mC*mC*mU | |
| WV- | 542 | CUCUUUCCAUGGAAGG | mC*SfU*SfC*SfU*SfU*SfU*SfC*SfC*SfA*SfU*SfG*Sf | SSSSSSSSSSSSSSSSSXX |
| 36901 | | UUCUAAACCAUCCU | G*SfA*SfA*SfG*SmG*SmU*SmU*mC*mU*mA*mA* | XXXXXXXXXX |
| | | | mA*C*C*A*mU*mC*mC*mU | |
| WV- | 543 | CUCUUUCCAUGGAAGG | mC*SfU*SfC*SfU*SfU*SfU*SfC*SfC*SfA*SfU*SfG*Sf | SSSSSSSSSSSSSSSSSSX |
| 36902 | | UUCUAAACCAUCCU | G*SfA*SfA*SfG*SmG*SmU*SmU*SmC*mU*mA*mA* | XXXXXXXXXX |
| | | | mA*C*C*A*mU*mC*mC*mU | |
| WV- | 544 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36903 | | UUCUAAACCAUCCU | G*SfA*SfA*SfG*SmG*SmU*SmU*SmC*SmU*mA*mA | XXXXXXXXXX |
| | | | *mA*C*C*A*mU*mC*mC*mU | |
| WV- | 545 | CUCUUUCCAUGGAAGG | mC*SfU*SfC*SfU*SfU*SfU*SfC*SfC*SfA*SfU*SfG*Sf | SSSSSSSSSSSSSSSSSSSSXX |
| 36904 | | UUCUAAACCAUCCU | G*SfA*SfA*SfG*SmG*SmU*SmU*SmC*SmU*SmA*m | XXXXXXX |
| | | | A*mA*C*C*A*mU*mC*mC*mU | |
| WV- | 546 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36905 | | UUCUAAACCAUCCU | G*SfA*SfA*SfG*SmG*SmU*SmU*SmC*SmU*SmA*S | SSXXXXXXXX |
| | | | mA*mA*C*C*A*mU*mC*mC*mU | |
| WV- | 547 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36906 | | UUCUAAACCAUCCU | G*SfA*SfA*SfG*SmG*SmU*SmU*SmC*SmU*SmA*S | SSSXXXXXXX |
| | | | mA*SmA*C*C*A*mU*mC*mC*mU | |
| WV- | 548 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36907 | | UUCUAAACCAUCCU | G*SfA*SfA*SfG*SmG*SmU*SmU*SmC*SmU*SmA*S | SSSSXXXXXX |
| | | | mA*SmA*SC*C*A*mU*mC*mC*mU | |
| WV- | 549 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36908 | | UUCUAAACCAUCCU | G*SfA*SfA*SfG*SmG*SmU*SmU*SmC*SmU*SmA*S | SSSSSXXXXX |
| | | | mA*SmA*SC*SC*A*mU*mC*mC*mU | |
| WV- | 550 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36909 | | UUCUAAACCAUCCU | G*SfA*SfA*SfG*SmG*SmU*SmU*SmC*SmU*SmA*S | SSSSSSXXXX |
| | | | mA*SmA*SC*SC*SA*mU*mC*mC*mU | |
| WV- | 551 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36910 | | UUCUAAACCAUCCU | G*SfA*SfA*SfG*SmG*SmU*SmU*SmC*SmU*SmA*S | SSSSSSSXXX |
| | | | mA*SmA*SC*SC*SA*SmU*mC*mC*mU | |
| WV- | 552 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36913 | | UUCUAAACCAUCCU | G*SfA*SfA*SfG*SmG*SmU*SmU*SmC*SmU*SmA*S | SSSSSSSSSS |
| | | | mA*SmA*SC*SC*SA*SmU*SmC*SmC*SmU | |
| WV- | 553 | CUCUUUCCAUGGAAGG | mC*fU*SfC*SfU*SfU*SfU*SfC*SfC*SfA*SfU*SfG*Sf | XSSSSSSSSSSSSSSSSSS |
| 36914 | | UUCUAAACCAUCCU | G*SfA*SfA*SfG*SmG*SmU*SmU*SmC*SmU*SmA*S | SSSSSSSSSS |
| | | | mA*SmA*SC*SC*SA*SmU*SmC*SmC*SmU | |
| WV- | 554 | CUCUUUCCAUGGAAGG | mC*fU*fC*SfU*SfU*SfU*SfC*SfC*SfA*SfU*SfG*SfG | XXSSSSSSSSSSSSSSSSS |
| 36915 | | UUCUAAACCAUCCU | *SfA*SfA*SfG*SmG*SmU*SmU*SmC*SmU*SmA*Sm | SSSSSSSSSS |
| | | | A*SmA*SC*SC*SA*SmU*SmC*SmC*SmU | |
| WV- | 555 | CUCUUUCCAUGGAAGG | mC*fU*fC*fU*SfU*SfU*SfC*SfC*SfA*SfU*SfG*SfG* | XXXSSSSSSSSSSSSSSSS |
| 36916 | | UUCUAAACCAUCCU | SfA*SfA*SfG*SmG*SmU*SmU*SmC*SmU*SmA*SmA | SSSSSSSSSS |
| | | | *SmA*SC*SC*SA*SmU*SmC*SmC*SmU | |
| WV- | 556 | CUCUUUCCAUGGAAGG | mC*fU*fC*fU*fU*SfU*SfC*SfC*SfA*SfU*SfG*SfG*Sf | XXXXSSSSSSSSSSSSSSS |
| 36917 | | UUCUAAACCAUCCU | A*SfA*SfG*SmG*SmU*SmU*SmC*SmU*SmA*SmA*S | SSSSSSSSSS |
| | | | mA*SC*SC*SA*SmU*SmC*SmC*SmU | |
| WV- | 557 | CUCUUUCCAUGGAAGG | mC*fU*fC*fU*fU*fU*SfC*SfC*SfA*SfU*SfG*SfG*SfA | XXXXXSSSSSSSSSSSSSS |
| 36918 | | UUCUAAACCAUCCU | *SfA*SfG*SmG*SmU*SmU*SmC*SmU*SmA*SmA*S | SSSSSSSSSS |
| | | | mA*SC*SC*SA*SmU*SmC*SmC*SmU | |
| WV- | 558 | CUCUUUCCAUGGAAGG | mC*fU*fC*fU*fU*fU*fC*SfC*SfA*SfU*SfG*SfG*SfA* | XXXXXXSSSSSSSSSSSSS |
| 36919 | | UUCUAAACCAUCCU | SfA*SfG*SmG*SmU*SmU*SmC*SmU*SmA*SmA*Sm | SSSSSSSSSS |
| | | | A*SC*SC*SA*SmU*SmC*SmC*SmU | |
| WV- | 559 | CUCUUUCCAUGGAAGG | mC*fU*fC*fU*fU*fU*fC*fC*SfA*SfU*SfG*SfG*SfA*S | XXXXXXXSSSSSSSSSSSS |
| 36920 | | UUCUAAACCAUCCU | fA*SfG*SmG*SmU*SmU*SmC*SmU*SmA*SmA*SmA | SSSSSSSSSS |
| | | | *SC*SC*SA*SmU*SmC*SmC*SmU | |
| WV- | 560 | CUCUUUCCAUGGAAGG | mC*fU*fC*fU*fU*fU*fC*fC*fA*SfU*SfG*SfG*SfA*Sf | XXXXXXXXSSSSSSSSSSS |
| 36921 | | UUCUAAACCAUCCU | A*SfG*SmG*SmU*SmU*SmC*SmU*SmA*SmA*SmA* | SSSSSSSSSS |
| | | | SC*SC*SA*SmU*SmC*SmC*SmU | |
| WV- | 561 | CUCUUUCCAUGGAAGG | mC*fU*fC*fU*fU*fU*fC*fC*fA*fU*SfG*SfG*SfA*SfA | XXXXXXXXXSSSSSSSSSS |
| 36922 | | UUCUAAACCAUCCU | *SfG*SmG*SmU*SmU*SmC*SmU*SmA*SmA*SmA*S | SSSSSSSSSS |
| | | | C*SC*SA*SmU*SmC*SmC*SmU | |
| WV- | 562 | CUCUUUCCAUGGAAGG | mC*fU*fC*fU*fU*fU*fC*fC*fA*fU*fG*SfG*SfA*SfA* | XXXXXXXXXXSSSSSSSSS |
| 36923 | | UUCUAAACCAUCCU | SfG*SmG*SmU*SmU*SmC*SmU*SmA*SmA*SmA*SC | SSSSSSSSSS |
| | | | *SC*SA*SmU*SmC*SmC*SmU | |
| WV- | 563 | CUCUUUCCAUGGAAGG | mC*fU*fC*fU*fU*fU*fC*fC*fA*fU*fG*fG*SfA*SfA*S | XXXXXXXXXXXSSSSSSSS |
| 36924 | | UUCUAAACCAUCCU | fG*SmG*SmU*SmU*SmC*SmU*SmA*SmA*SmA*SC* | SSSSSSSSSS |
| | | | SC*SA*SmU*SmC*SmC*SmU | |
| WV- | 564 | CUCUUUCCAUGGAAGG | mC*fU*fC*fU*fU*fU*fC*fC*fA*fU*fG*fG*fA*SfA*Sf | XXXXXXXXXXXXSSSSSSS |
| 36925 | | UUCUAAACCAUCCU | G*SmG*SmU*SmU*SmC*SmU*SmA*SmA*SmA*SC* | SSSSSSSSSS |
| | | | SC*SA*SmU*SmC*SmC*SmU | |
| WV- | 565 | CUCUUUCCAUGGAAGG | mC*fU*fC*fU*fU*fU*fC*fC*fA*fU*fG*fG*fA*fA*SfG | XXXXXXXXXXXXXSSSSSS |
| 36926 | | UUCUAAACCAUCCU | *SmG*SmU*SmU*SmC*SmU*SmA*SmA*SmA*SC*SC | SSSSSSSSSS |
| | | | *SA*SmU*SmC*SmC*SmU | |
| WV- | 566 | CUCUUUCCAUGGAAGG | mC*fU*fC*fU*fU*fU*fC*fC*fA*fU*fG*fG*fA*fA*fG* | XXXXXXXXXXXXXXSSSS |
| 36927 | | UUCUAAACCAUCCU | SmG*SmU*SmU*SmC*SmU*SmA*SmA*SmA*SC*SC* | SSSSSSSSSSS |
| | | | SA*SmU*SmC*SmC*SmU | |
| WV- | 567 | CUCUUUCCAUGGAAGG | mC*fU*fC*fU*fU*fU*fC*fC*fA*fU*fG*fG*fA*fA*fG* | XXXXXXXXXXXXXXXSSS |
| 36928 | | UUCUAAACCAUCCU | mG*SmU*SmU*SmC*SmU*SmA*SmA*SmA*SC*SC*S | SSSSSSSSSSS |
| | | | A*SmU*SmC*SmC*SmU | |
| WV- | 568 | CUCUUUCCAUGGAAGG | mC*fU*fC*fU*fU*fU*fC*fC*fA*fU*fG*fG*fA*fA*fG* | XXXXXXXXXXXXXXXXSS |
| 36929 | | UUCUAAACCAUCCU | mG*mU*SmU*SmC*SmU*SmA*SmA*SmA*SC*SC*S | SSSSSSSSSSS |
| | | | A*SmU*SmC*SmC*SmU | |
| WV- | 569 | CUCUUUCCAUGGAAGG | mC*fU*fC*fU*fU*fU*fC*fC*fA*fU*fG*fG*fA*fA*fG* | XXXXXXXXXXXXXXXXX |
| 36930 | | UUCUAAACCAUCCU | mG*mU*mU*SmC*SmU*SmA*SmA*SmA*SC*SC*SA | SSSSSSSSSSSS |
| | | | *SmU*SmC*SmC*SmU | |
| WV- | 570 | CUCUUUCCAUGGAAGG | mC*fU*fC*fU*fU*fU*fC*fC*fA*fU*fG*fG*fA*fA*fG* | XXXXXXXXXXXXXXXXX |
| 36931 | | UUCUAAACCAUCCU | mG*mU*mU*mC*SmU*SmA*SmA*SmA*SC*SC*SA* | XSSSSSSSSSSS |
| | | | SmU*SmC*SmC*SmU | |
| WV- | 571 | CUCUUUCCAUGGAAGG | mC*fU*fC*fU*fU*fU*fC*fC*fA*fU*fG*fG*fA*fA*fG* | XXXXXXXXXXXXXXXXX |
| 36932 | | UUCUAAACCAUCCU | mG*mU*mU*mC*mU*SmA*SmA*SmA*SC*SC*SA*S | XXSSSSSSSSSS |
| | | | mU*SmC*SmC*SmU | |
| WV- | 572 | CUCUUUCCAUGGAAGG | mC*fU*fC*fU*fU*fU*fC*fC*fA*fU*fG*fG*fA*fA*fG* | XXXXXXXXXXXXXXXXX |
| 36933 | | UUCUAAACCAUCCU | mG*mU*mU*mC*mU*mA*SmA*SmA*SC*SC*SA*Sm | XXXSSSSSSSSS |
| | | | U*SmC*SmC*SmU | |
| WV- | 573 | CUCUUUCCAUGGAAGG | mC*fU*fC*fU*fU*fU*fC*fC*fA*fU*fG*fG*fA*fA*fG* | XXXXXXXXXXXXXXXXX |
| 36934 | | UUCUAAACCAUCCU | mG*mU*mU*mC*mU*mA*mA*SmA*SC*SC*SA*SmU | XXXXSSSSSSSS |
| | | | *SmC*SmC*SmU | |
| WV- | 574 | CUCUUUCCAUGGAAGG | mC*fU*fC*fU*fU*fU*fC*fC*fA*fU*fG*fG*fA*fA*fG* | XXXXXXXXXXXXXXXXX |
| 36935 | | UUCUAAACCAUCCU | mG*mU*mU*mC*mU*mA*mA*mA*SC*SC*SA*SmU* | XXXXXSSSSSSS |
| | | | SmC*SmC*SmU | |
| WV- | 575 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36936 | | UUCUAAACCAUCCU | G*SfA*SfA*SfG*SmG*SmU*SmU*SmC*SmU*SmA*S | SSSSSSOSSS |
| | | | mA*SmA*SC*SC*SAmU*SmC*SmC*SmU | |
| WV- | 576 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36937 | | UUCUAAACCAUCCU | G*SfA*SfA*SfG*SmG*SmU*SmU*SmC*SmU*SmA*S | SSSSSOOSSS |
| | | | mA*SmA*SC*SCAmU*SmC*SmC*SmU | |
| WV- | 577 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36938 | | UUCUAAACCAUCCU | G*SfA*SfA*SfG*SmG*SmU*SmU*SmC*SmU*SmA*S | SSSSOOOSSS |
| | | | mA*SmA*SCCAmU*SmC*SmC*SmU | |
| WV- | 578 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36939 | | UUCUAAACCAUCCU | G*SfA*SfA*SfG*SmG*SmU*SmU*SmC*SmU*SmA*S | SSSOOOOSSS |
| | | | mA*SmACCAmU*SmC*SmC*SmU | |
| WV- | 579 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36940 | | UUCUAAACCAUCCU | G*SfA*SfA*SfG*SmG*SmU*SmU*SmC*SmU*SmA*S | SSOOOOOSSS |
| | | | mAmACCAmU*SmC*SmC*SmU | |
| WV- | 580 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36941 | | UUCUAAACCAUCCU | G*SfA*SfA*SfG*SmG*SmU*SmU*_{SmC}*SmU*SmAmA | SOOOOOOSSS |
| | | | mACCAmU*SmC*SmC*SmU | |
| WV- | 581 | CUCUUUCCAUGGAAGG | mC*SfU*SfC*SfU*SfU*SfU*SfC*SfC*SfA*SfU*SfG*Sf | SSSSSSSSSSSSSSSSSSS |
| 36942 | | UUCUAAACCAUCCU | G*SfA*SfA*SfG*SmG*SmU*SmU*SmC*SmUmAmAm | OOOOOOOSSS |
| | | | ACCAmU*SmC*SmC*SmU | |
| WV- | 582 | CUCUUUCCAUGGAAGG | mC*SfU*SfC*SfU*SfU*SfU*SfC*SfC*SfA*SfU*SfG*Sf | SSSSSSSSSSSSSSSSSSO |
| 36943 | | UUCUAAACCAUCCU | G*SfA*SfA*SfG*SmG*SmU*SmU*SmCmUmAmAmA | OOOOOOOSSS |
| | | | CCAmU*SmC*SmC*SmU | |
| WV- | 583 | CUCUUUCCAUGGAAGG | mC*SfU*SfC*SfU*SfU*SfU*SfC*SfC*SfA*SfU*SfG*Sf | SSSSSSSSSSSSSSSSSOO |
| 36944 | | UUCUAAACCAUCCU | G*SfA*SfA*SfG*SmG*SmU*SmUmCmUmAmAmACC | OOOOOOOSSS |
| | | | AmU*SmC*SmC*SmU | |
| WV- | 584 | CUCUUUCCAUGGAAGG | mC*SfU*SfC*SfU*SfU*SfU*SfC*SfC*SfA*SfU*SfG*Sf | SSSSSSSSSSSSSSSSOOO |
| 36945 | | UUCUAAACCAUCCU | G*SfA*SfA*SfG*SmG*SmUmUmCmUmAmAmACCA | OOOOOOOSSS |
| | | | mU*SmC*SmC*SmU | |
| WV- | 585 | CUCUUUCCAUGGAAGG | mC*SfU*SfC*SfU*SfU*SfU*SfC*SfC*SfA*SfU*SfG*Sf | SSSSSSSSSSSSSSSOOOO |
| 36946 | | UUCUAAACCAUCCU | G*SfA*SfA*SfG*SmGmUmUmCmUmAmAmACCAmU | OOOOOOOSSS |
| | | | *SmC*SmC*SmU | |
| WV- | 586 | CUCUUUCCAUGGAAGG | mC*SfU*SfC*SfU*SfU*SfU*SfC*SfC*SfA*SfU*SfG*Sf | SSSSSSSSSSSSSSOOOOO |
| 36947 | | UUCUAAACCAUCCU | G*SfA*SfA*SfGmGmUmUmCmUmAmAmACCAmU*S | OOOOOOOSSS |
| | | | mC*SmC*SmU | |
| WV- | 587 | CUCUUUCCAUGGAAGG | mC*SfU*SfC*SfU*SfU*SfU*SfC*SfC*SfA*SfU*SfG*Sf | SSSSSSSSSSSSSOOOOOO |
| 36948 | | UUCUAAACCAUCCU | G*SfA*SfAfGmGmUmUmCmUmAmAmACCAmU*Sm | OOOOOOOSSS |
| | | | C*SmC*SmU | |
| WV- | 588 | CUCUUUCCAUGGAAGG | mC*SfU*SfC*SfU*SfU*SfU*SfC*SfC*SfA*SfU*SfG*Sf | SSSSSSSSSSSSOOOOOOO |
| 36949 | | UUCUAAACCAUCCU | G*SfAfAfGmGmUmUmCmUmAmAmACCAmU*SmC* | OOOOOOOSSS |
| | | | SmC*SmU | |
| WV- | 589 | CUCUUUCCAUGGAAGG | mC*SfU*SfC*SfU*SfU*SfU*SfC*SfC*SfA*SfU*SfG*Sf | SSSSSSSSSSSOOOOOOOO |
| 36950 | | UUCUAAACCAUCCU | GfAfAfGmGmUmUmCmUmAmAmACCAmU*SmC*Sm | OOOOOOOSSS |
| | | | C*SmU | |
| WV- | 590 | CUCUUUCCAUGGAAGG | mC*SfU*SfC*SfU*SfU*SfU*SfC*SfC*SfA*SfU*SfGfGf | SSSSSSSSSSOOOOOOOOO |
| 36951 | | UUCUAAACCAUCCU | AfAfGmGmUmUmCmUmAmAmACCAmU*SmC*SmC* | OOOOOOOSSS |
| | | | SmU | |
| WV- | 591 | CUCUUUCCAUGGAAGG | mC*SfU*SfC*SfU*SfU*SfU*SfC*SfC*SfA*SfUfGfGfAf | SSSSSSSSSOOOOOOOOOO |
| 36952 | | UUCUAAACCAUCCU | AfGmGmUmUmCmUmAmAmACCAmU*SmC*SmC*S | OOOOOOOSSS |
| | | | mU | |
| WV- | 592 | CUCUUUCCAUGGAAGG | mC*SfU*SfC*SfU*SfU*SfU*SfC*SfC*SfAfUfGfGfAfAf | SSSSSSSSOOOOOOOOOOO |
| 36953 | | UUCUAAACCAUCCU | GmGmUmUmCmUmAmAmACCAmU*SmC*SmC*SmU | OOOOOOOSSS |
| WV- | 593 | CUCUUUCCAUGGAAGG | mC*SfU*SfC*SfU*SfU*SfU*SfC*SfCfAfUfGfGfAfAfG | SSSSSSSOOOOOOOOOOOO |
| 36954 | | UUCUAAACCAUCCU | mGmUmUmCmUmAmAmACCAmU*SmC*SmC*SmU | OOOOOOOSSS |
| WV- | 594 | CUCUUUCCAUGGAAGG | mC*SfU*SfC*SfU*SfU*SfU*SfCfCfAfUfGfGfAfAfGmG | SSSSSSOOOOOOOOOOOOO |
| 36955 | | UUCUAAACCAUCCU | mUmUmCmUmAmAmACCAmU*SmC*SmC*SmU | OOOOOOOSSS |
| WV- | 595 | CUCUUUCCAUGGAAGG | mC*fU*fC*fU*fU*fU*fC*fC*fA*fU*fG*fG*fA*fA*fG* | XXXXXXXXXXXXXXXXX |
| 36958 | | UUCUAAACCAUCCU | mG*mU*mU*mC*mU*mA*mA*mA*C*C*AmU*mC*m | XXXXXXXXOXXX |
| | | | C*mU | |
| WV- | 596 | CUCUUUCCAUGGAAGG | mC*fU*fC*fU*fU*fU*fC*fC*fA*fU*fG*fG*fA*fA*fG* | XXXXXXXXXXXXXXXXX |
| 36959 | | UUCUAAACCAUCCU | mG*mU*mU*mC*mU*mA*mA*mA*C*CAmU*mC*mC | XXXXXXXOOXXX |
| | | | *mU | |
| WV- | 597 | CUCUUUCCAUGGAAGG | mC*fU*fC*fU*fU*fU*fC*fC*fA*fU*fG*fG*fA*fA*fG* | XXXXXXXXXXXXXXXXX |
| 36960 | | UUCUAAACCAUCCU | mG*mU*mU*mC*mU*mA*mA*mA*CCAmU*mC*mC* | XXXXXXOOOXXX |
| | | | mU | |
| WV- | 598 | CUCUUUCCAUGGAAGG | mC*fU*fC*fU*fU*fU*fC*fC*fA*fU*fG*fG*fA*fA*fG* | XXXXXXXXXXXXXXXXX |
| 36961 | | UUCUAAACCAUCCU | mG*mU*mU*mC*mU*mA*mA*mACCAmU*mC*mC* | XXXXXOOOOXXX |
| | | | mU | |
| WV- | 599 | CUCUUUCCAUGGAAGG | mC*fU*fC*fU*fU*fU*fC*fC*fA*fU*fG*fG*fA*fA*fG* | XXXXXXXXXXXXXXXXX |
| 36962 | | UUCUAAACCAUCCU | mG*mU*mU*mC*mU*mA*mAmACCAmU*mC*mC*m | XXXXOOOOOXXX |
| | | | u | |
| WV- | 600 | CUCUUUCCAUGGAAGG | mC*fU*fC*fU*fU*fU*fC*fC*fA*fU*fG*fG*fA*fA*fG* | XXXXXXXXXXXXXXXXX |
| 36963 | | UUCUAAACCAUCCU | mG*mU*mU*mC*mU*mAmAmACCAmU*mC*mC*mU | XXXOOOOOOXXX |
| WV- | 601 | CUCUUUCCAUGGAAGG | mC*fU*fC*fU*fU*fU*fC*fC*fA*fU*fG*fG*fA*fA*fG* | XXXXXXXXXXXXXXXXX |
| 36964 | | UUCUAAACCAUCCU | mG*mU*mU*mC*mUmAmAmACCAmU*mC*mC*mU | XXOOOOOOOXXX |
| WV- | 602 | CUCUUUCCAUGGAAGG | mC*fU*fC*fU*fU*fU*fC*fC*fA*fU*fG*fG*fA*fA*fG* | XXXXXXXXXXXXXXXXX |
| 36965 | | UUCUAAACCAUCCU | mG*mU*mU*mCmUmAmAmACCAmU*mC*mC*mU | XOOOOOOOOXXX |
| WV- | 603 | CUCUUUCCAUGGAAGG | mC*fU*fC*fU*fU*fU*fC*fC*fA*fU*fG*fG*fA*fA*fG* | XXXXXXXXXXXXXXXXX |
| 36966 | | UUCUAAACCAUCCU | mG*mU*mUmCmUmAmAmACCAmU*mC*mC*mU | OOOOOOOOOXXX |
| WV- | 604 | CUCUUUCCAUGGAAGG | mC*fU*fC*fU*fU*fU*fC*fC*fA*fU*fG*fG*fA*fA*fG* | XXXXXXXXXXXXXXXXO |
| 36967 | | UUCUAAACCAUCCU | mG*mUmUmCmUmAmAmACCAmU*mC*mC*mU | OOOOOOOOOXXX |
| WV- | 605 | CUCUUUCCAUGGAAGG | mC*fU*fC*fU*fU*fU*fC*fC*fA*fU*fG*fG*fA*fA*fG* | XXXXXXXXXXXXXXXOO |
| 36968 | | UUCUAAACCAUCCU | mGmUmUmCmUmAmAmACCAmU*mC*mC*mU | OOOOOOOOOXXX |
| WV- | 606 | CUCUUUCCAUGGAAGG | mC*fU*fC*fU*fU*fU*fC*fC*fA*fU*fG*fG*fA*fA*fGm | XXXXXXXXXXXXXXOOO |
| 36969 | | UUCUAAACCAUCCU | GmUmUmCmUmAmAmACCAmU*mC*mC*mU | OOOOOOOOOXXX |
| WV- | 607 | CUCUUUCCAUGGAAGG | mC*fU*fC*fU*fU*fU*fC*fC*fA*fU*fG*fG*fA*fAfGm | XXXXXXXXXXXXXOOOO |
| 36970 | | UUCUAAACCAUCCU | GmUmUmCmUmAmAmACCAmU*mC*mC*mU | OOOOOOOOOXXX |
| WV- | 608 | CUCUUUCCAUGGAAGG | mC*fU*fC*fU*fU*fU*fC*fC*fA*fU*fG*fG*fAfAfGmG | XXXXXXXXXXXXOOOOO |
| 36971 | | UUCUAAACCAUCCU | mUmUmCmUmAmAmACCAmU*mC*mC*mU | OOOOOOOOOXXX |
| WV- | 609 | CUCUUUCCAUGGAAGG | mC*fU*fC*fU*fU*fU*fC*fC*fA*fU*fG*fGfAfAfGmGm | XXXXXXXXXXXOOOOOO |
| 36972 | | UUCUAAACCAUCCU | UmUmCmUmAmAmACCAmU*mC*mC*mU | OOOOOOOOOXXX |
| WV- | 610 | CUCUUUCCAUGGAAGG | mC*fU*fC*fU*fU*fU*fC*fC*fA*fU*fGfGfAfAfGmGm | XXXXXXXXXXOOOOOOO |
| 36973 | | UUCUAAACCAUCCU | UmUmCmUmAmAmACCAmU*mC*mC*mU | OOOOOOOOOXXX |
| WV- | 611 | CUCUUUCCAUGGAAGG | mC*fU*fC*fU*fU*fU*fC*fC*fA*fUfGfGfAfAfGmGmU | XXXXXXXXXOOOOOOOO |
| 36974 | | UUCUAAACCAUCCU | mUmCmUmAmAmACCAmU*mC*mC*mU | OOOOOOOOOXXX |
| WV- | 612 | CUCUUUCCAUGGAAGG | mC*fU*fC*fU*fU*fU*fC*fC*fAfUfGfGfAfAfGmGmUm | XXXXXXXXOOOOOOOOO |
| 36975 | | UUCUAAACCAUCCU | UmCmUmAmAmACCAmU*mC*mC*mU | OOOOOOOOOXXX |
| WV- | 613 | CUCUUUCCAUGGAAGG | mC*fU*fC*fU*fU*fU*fC*fCfAfUfGfGfAfAfGmGmUm | XXXXXXXOOOOOOOOOO |
| 36976 | | UUCUAAACCAUCCU | UmCmUmAmAmACCAmU*mC*mC*mU | OOOOOOOOOXXX |
| WV- | 614 | CUCUUUCCAUGGAAGG | mC*fU*fC*fU*fU*fU*fCfCfAfUfGfGfAfAfGmGmUmU | XXXXXXOOOOOOOOOOO |
| 36977 | | UUCUAAACCAUCCU | mCmUmAmAmACCAmU*mC*mC*mU | OOOOOOOOOXXX |

TABLE 1C
Example oligonucleotides and/or compositions that target SERPINA1.

| | SEQ | Base | | Stereochemistry/ |
|---|---|---|---|---|
| ID | ID NO | Sequence | Description | Linkage |
| WV- | 615 | GCCCCAGCAGCA | mG * mC * mC * rC * rC * rA * rG * | XXXXX XXXXX |
| 23395 | | UCACUCCCUUUC | rC * rA * rG * rC * rA * rU * rC * | XXXXX XXXXX |
| | | UCGUCGAU | rA * rC * rU * rC * rC * rC * rU * | XXXXX XXXXX X |
| | | | rU * rU * rC * rU * rC * rG * rU * | |
| | | | rC * mG * mA * mU | |
| WV- | 616 | CCCCAGCAGCAU | fC * fC * fC * fC * fA * fG * fC * | XXXXX XXXXX |
| 23932 | | CACUCCCUUUCT | fA * fG * fC * fA * fU * fC * fA * | XXXXX XXXXX |
| | | CGUCGA | fC * mU * mC * mC * mC * mU * mU * | XXXXX XXXX |
| | | | mU * mC * T * C * G * mU * | |
| | | | mC * mG * mA | |
| WV- | 617 | GCCCCAGCAGCA | mG * mC * mC * rC * rC * rA * rG * | XXXXX XXXXX |
| 27817 | | UCACUCCCUUUC | rC * rA * rG * rC * rA * rU * rC * | XXXXX XXXXX |
| | | UCIUCGAU | rA * rC * rU * rC * rC * rC * rU * | XXXXX XXXXX X |
| | | | rU * rU * rC * rU * rC * I * rU * | |
| | | | rC * mG * mA * mU | |
| WV- | 618 | CCCCAGCAGCAU | fC * fC * fC * fC * fA * fG * fC * | XXXXX XXXXX |
| 27818 | | CACUCCCUUUCT | fA * fG * fC * fA * fU * fC * fA * | XXXXX XXXXX |
| | | CIUCGA | fC * mU * mC * mC * mC * mU * mU * | XXXXX XXXX |
| | | | mU * mC * T * C * I * mU * | |
| | | | mC * mG * mA | |
| WV- | 619 | GCCCCAGCAGCA | fG * fC * fC * fC * fC * fA * fG * | XXXXX XXXXX |
| 27819 | | UCACUCCCUUUC | fC * fA * fG * fC * fA * fU * fC * | XXXXX XXXXX |
| | | TCGUCGAU | fA * fC * mU * mC * mC * mC * mU * | XXXXX XXXXX X |
| | | | mU * mU * mC * T * C * G * | |
| | | | mU * mC * mG * mA * mU | |
| WV- | 620 | GCCCCAGCAGCA | fG * fC * fC * fC * fC * fA * fG * | XXXXX XXXXX |
| 27820 | | UCACUCCCUUUC | fC * fA * fG * fC * fA * fU * fC * | XXXXX XXXXX |
| | | TCIUCGAU | fA * fC * mU * mC * mC * mC * mU * | XXXXX XXXXX X |
| | | | mU * mU * mC * T * C * I * | |
| | | | mU * mC * mG * mA * mU | |
| WV- | 621 | CCCCAGCAGCAU | mC * mC * mC * rC * rA * rG * rC * | XXXXX XXXXX |
| 27821 | | CACUCCCUUUCU | rA * rG * rC * rA * rU * rC * rA * | XXXXX XXXXX |
| | | CGUCGA | rC * rU * rC * rC * rC * rU * rU * | XXXXX XXXX |
| | | | rU * rC * rU * rC * rG * rU * mC * | |
| | | | mG * mA | |
| WV- | 622 | CCCCAGCAGCAU | mC * mC * mC * rC * rA * rG * rC * | XXXXX XXXXX |
| 27822 | | CACUCCCUUUCU | rA * rG * rC * rA * rU * rC * rA * | XXXXX XXXXX |
| | | CIUCGA | rC * rU * rC * rC * rC * rU * rU * | XXXXX XXXX |
| | | | rU * rC * rU * rC * I * rU * mC * | |
| | | | mG * mA | |
| WV- | 623 | GCCCCAGCAGCA | fG * fC * fC * fC * fC * fA * fG * | XXXXX XXXXX |
| 28177 | | UCACUCCCUUUC | fC * fA * fG * fC * fA * fU * fC * | XXXXX XXXXX |
| | | TCGUCGAU | fA * fC * mU * mC * mC * mC * mU * | XXXXX XXXXX X |
| | | | mU * mU * mC * T * b001C * | |
| | | | G * mU * mC * mG * mA * mU | |
| WV- | 624 | CCCCAGCAGCAU | fC * fC * fC * fC * fA * fG * fC * | XXXXX XXXXX |
| 28179 | | CACUCCCUUUCT | fA * fG * fC * fA * fU * fC * fA * | XXXXX XXXXX |
| | | CGUCGA | fC * mU * mC * mC * mC * mU * mU * | XXXXX XXXX |
| | | | mU * mC * T * b001C * G * | |
| | | | mU * mC * mG * mA | |

TABLE 1D
Example oligonucleotides and/or compositions.

| | SEQ | | | | |
|---|---|---|---|---|---|
| | ID | | Base | Stereochemistry/ | |
| ID | NO | Description | Sequence | Linkage | Target |
| WV- | 625 | fC*fU*fC*fU*fU*fU*fC*fC*A*fU*G*fG*fA*fA*fG*mG* | CUCUUUCCAUGGA | XXXXXXXXXXXX | LUC |
| 23873 | | mU*mU*mC*mU*mA*mA*mA*C*C*A*mU*mC*mC*mU | AGGUUCUAAACCA | XXXXXXXXXXXX | |
| | | | UCCU | XXXXX | |
| WV- | 626 | fC*SfU*SfC*SfU*SfU*SfU*SfC*SfC*SfA*SfU*SfG*SfG* | CUCUUUCCAUGGA | SSSSSSSSSSSSSSS | LUC |
| 24111 | | SfA*SfA*SfG*SmG*SmU*SmU*SmC*SmU*SmA*SmA*SmA* | AGGUUCUAAACCA | SSSSSSSSSSSSSS | |
| | | SC*SC*SA*SmU*SmC*SmC*SmU | UCCU | | |
| WV- | 627 | fC*SfC*SfA*SfA*SfC*SfC*SfA*SfG*SfA*SfA*SfA*SfU* | CCAACCAGAAAUU | SSSSSSSSSSSSSSS | EEF1A1 |
| 31442 | | SfU*SfG*SfG*SmC*SmA*SmC*SmA*SmA*SmA*SmU*SmG* | GGCACAAAUGCCA | SSSSSSSSSRRSSS | |
| | | SfC*SC*RA*RmC*SmU*SmG*SmU | CUGU | | |
| WV- | 628 | fC*fC*fA*fA*fC*fC*fA*fG*fA*fA*fA*fU*fU*fG*fG*mC* | CCAACCAGAAAUU | XXXXXXXXXXXX | EEF1A1 |
| 31443 | | mA*mC*mA*mA*mA*mU*mG*fC*C*A*mC*mU*mG*mU | GGCACAAAUGCCA | XXXXXXXXXXXX | |
| | | | CUGU | XXXXX | |
| WV- | 629 | fU*SfG*SfA*SfG*SfG*SfC*SfG*SfA*SfA*SfG*SfC*SfA* | UGAGGCGAAGCAU | SSSSSSSSSSSSSSS | HSP90B1 |
| 31448 | | SfU*SfU*SfC*SmU*SmU*SmU*SmC*SmU*SmA*SmU*SmU* | UCUUUCUAUUCCA | SSSSSSSSSRRSSS | |
| | | SfC*SC*RA*RmU*SmC*SmU*SmC | UCUC | | |
| WV- | 630 | fU*fG*fA*fG*fG*fC*fG*fA*fA*fG*fC*fA*fU*fU*fC*mU* | UGAGGCGAAGCAU | XXXXXXXXXXXX | HSP90B1 |
| 31449 | | mU*mU*mC*mU*mA*mU*mU*fC*C*A*mU*mC*mU*mC | UCUUUCUAUUCCA | XXXXXXXXXXXX | |
| | | | UCUC | XXXXX | |
| WV- | 631 | fC*SfU*SfC*SfU*SfG*SfU*SfA*SfG*SfU*SfC*SfU*SfG* | CUCUGUAGUCUGG | SSSSSSSSSSSSSSS | GATM |
| 31451 | | SfG*SfA*SfG*SmC*SmA*SmA*SmG*SmA*SmU*SmG*SmC* | AGCAAGAUGCCCA | SSSSSSSSSRRSSS | |
| | | SfC*SC*RA*RmC*SmG*SmC*SmA | CGCA | | |
| WV- | 632 | fC*fU*fC*fU*fG*fU*fA*fG*fU*fC*fU*fG*fG*£A*fG*mC* | CUCUGUAGUCUGG | XXXXXXXXXXXX | GATM |
| 31452 | | mA*mA*mG*mA*mU*mG*mC*fC*C*A*mC*mG*mC*mA | AGCAAGAUGCCCA | XXXXXXXXXXXX | |
| | | | CGCA | XXXXX | |
| WV- | 633 | fU*SfG*SfC*SfC*SfC*SfU*SfG*SfA*SfA*Sfu*SfU*SfC* | UGCCCUGAAUUCC | SSSSSSSSSSSSSSS | HSP90AB1 |
| 31454 | | SfC*SfA*SfA*SmC*SmU*SmG*SmA*SmC*SmC*SmU*SmU* | AACUGACCUUCCA | SSSSSSSSSRRSSS | |
| | | SfC*SC*RA*RmC*SmA*SmG*SmA | CAGA | | |
| WV- | 634 | fU*fG*fC*fC*fC*fU*fG*fA*fA*fU*fU*fC*fC*fA*fA*mC* | UGCCCUGAAUUCC | XXXXXXXXXXXX | HSP90AB1 |
| 31455 | | mU*mG*mA*mC*mC*mU*mU*fC*C*A*mC*mA*mG*mA | AACUGACCUUCCA | XXXXXXXXXXXX | |
| | | | CAGA | XXXXX | |
| WV- | 635 | fC*SfA*SfG*SfA*SfA*SfG*SfG*SfA*SfA*SfC*SfA*SfU* | CAGAAGGAACAUG | SSSSSSSSSSSSSSS | GHITM |
| 31475 | | SfG*SfC*SfU*SmG*SmA*SmA*SmA*SmA*SmG*SmA*SmA* | CUGAAAAGAACCA | SSSSSSSSSRRSSS | |
| | | SfC*SC*RA*RmA*SmU*SmC*SmC | AUCC | | |
| WV- | 636 | fC*fA*fG*fA*fA*fG*fG*fA*fA*fC*fA*fU*fG*fC*fU*mG* | CAGAAGGAACAUG | XXXXXXXXXXXX | GHITM |
| 31476 | | mA*mA*mA*mA*mG*mA*mA*fC*C*A*mA*mU*mC*mC | CUGAAAAGAACCA | XXXXXXXXXXXX | |
| | | | AUCC | XXXXX | |
| WV- | 637 | fA*SfU*SfC*SfC*SfA*SfC*SfU*SfG*SfU*SfG*SfG*SfC* | AUCCACUGUGGCA | SSSSSSSSSSSSSSS | UGP2 |
| 31484 | | SfA*SfC*SfC*SmC*SmA*SmG*SmA*SmU*SmU*SmA*SmU* | CCCAGAUUAUCCA | SSSSSSSSSRRSSS | |
| | | SfC*SC*RA*RmU*SmG*SmU*SmU | UGUU | | |
| WV- | 638 | fA*fU*fC*fC*fA*fC*fU*fG*fU*fG*fG*fC*fA*fC*fC*mC* | AUCCACUGUGGCA | XXXXXXXXXXXX | UGP2 |
| 31485 | | mA*mG*mA*mU*mU*mA*mU*fC*C*A*mU*mG*mU*mU | CCCAGAUUAUCCA | XXXXXXXXXXXX | |
| | | | UGUU | XXXXX | |
| WV- | 639 | fG*SfC*SfU*SfG*SfA*SfG*SfA*SfU*SfC*SfC*SfU*SfU* | GCUGAGAUCCUUA | SSSSSSSSSSSSSSS | EIF4H |
| 31523 | | SfA*SfA*SfA*SmG*SmA*SmU*SmA*SmG*SmC*SmA*SmU* | AAGAUAGCAUCCA | SSSSSSSSSRRSSS | |
| | | SfC*SC*RA*RmU*SmG*SmU*SmC | UGUC | | |
| WV- | 640 | fG*fC*fU*fG*fA*fG*fA*fU*fC*fC*fU*fU*fA*fA*fA*mG* | GCUGAGAUCCUUA | XXXXXXXXXXXX | EIF4H |
| 31524 | | mA*mU*mA*mG*mC*mA*mU*fC*C*A*mU*mG*mU*mC | AAGAUAGCAUCCA | XXXXXXXXXXXX | |
| | | | UGUC | XXXXX | |
| WV- | 641 | fU*SfU*SfA*SfA*SfU*SfC*SfC*SfA*SfU*SfC*SfU*SfC* | UUAAUCCAUCUCU | SSSSSSSSSSSSSSS | SRSF1 |
| 31535 | | SfU*SfU*SfC*SmA*SmG*SmA*SmU*SmA*SmU*SmG*SmU* | UCAGAUAUGUCCA | SSSSSSSSSRRSSS | |
| | | SfC*SC*RA*RmC*SmA*SmG*SmA | CAGA | | |
| WV- | 642 | fU*fU*fA*fA*fU*fC*fC*fA*fU*fC*fU*fC*fU*fU*fC*mA* | UUAAUCCAUCUCU | XXXXXXXXXXXX | SRSF1 |
| 31536 | | mG*mA*mU*mA*mU*mG*mU*fC*C*A*mC*mA*mG*mA | UCAGAUAUGUCCA | XXXXXXXXXXXX | |
| | | | CAGA | XXXXX | |
| WV- | 643 | fC*SfU*SfC*SfU*SfU*SfU*SfC*SfC*SfA*SfU*SfG*SfG* | CUCUUUCCAUGGA | SSSSSSSSSSSSSSS | LUC |
| 31939 | | SfA*SfA*SfG*SmG*SmU*SmU*SmC*SmU*SmA*SmA*SmA* | AGGUUCUAAACCA | SSSSSSSSSSnXSSn | |
| | | SC*SC*SAn001mU*SmC*SmCn001mU | UCCU | X | |
| WV- | 644 | fC*SfU*SfC*SfU*SfU*SfU*SfC*SfC*SfA*SfU*SfG*SfG* | CUCUUUCCAUGGA | SSSSSSSSSSSSSSS | LUC |
| 31940 | | SfA*SfA*SfG*SmG*SmU*SmU*SmC*SmUn001mA*SmA* | AGGUUCUAAACCA | SSSSnXSSSSSnXSS | |
| | | SmA*SC*SC*SAn001mU*SmC*SmCn001mU | UCCU | nX | |
| WV- | 645 | fC*SfU*SfCn001fU*SfU*SfU*SfC*SfC*SfA*SfU* | CUCUUUCCAUGGA | SSnXSSSSSSSnXSS | LUC |
| 31941 | | SfGn001fG*SfA*SfAn001fG*SmG*SmU*SmU*SmC*SmU* | AGGUUCUAAACCA | nXSSSSSSSSSSSSS | |
| | | SmA*SmA*SmA*SC*SC*SA*SmU*SmC*SmC*SmU | UCCU | SS | |
| WV- | 646 | fC*SfU*SfCn001fU*SfU*SfU*SfC*SfC*SfA*SfU* | CUCUUUCCAUGGA | SSnXSSSSSSSnXSS | LUC |
| 31942 | | SfGn001fG*SfA*SfA*SfG*SmG*SmU*SmU*SmC*SmUn001mA* | AGGUUCUAAACCA | SSSSSSnXSSSSSSS | |
| | | SmA*SmA*SC*SC*SA*SmU*SmC*SmC*SmU | UCCU | SS | |
| WV- | 647 | fC*SfU*SfCn001fU*SfU*SfU*SfC*SfC*SfA*SfU*SfG* | CUCUUUCCAUGGA | SSnXSSSSSSSSnXS | LUC |
| 31943 | | SfGn001fA*SfA*SfG*SmG*SmU*SmU*SmC*SmUn001mA* | AGGUUCUAAACCA | SSSSSSnXSSSSSSS | |
| | | SmA*SmA*SC*SC*SA*SmU*SmC*SmC*SmU | UCCU | SS | |
| WV- | 648 | fC*SfU*SfCn001fU*SfU*SfU*SfC*SfC*SfA*SfU*SfG* | CUCUUUCCAUGGA | SSnXSSSSSSSSnXS | LUC |
| 31944 | | SfGn001fA*SfA*SfG*SmG*SmU*SmU*SmC*SmU*SmA*SmA* | AGGUUCUAAACCA | SSSSSSSSSSSSnXS | |
| | | SmA*SC*SC*SAn001mU*SmC*SmC*SmU | UCCU | SS | |
| WV- | 649 | fC*SfU*SfCn001fU*SfU*SfU*SfC*SfC*SfA*SfU*SfG* | CUCUUUCCAUGGA | SSnXSSSSSSSSnXS | LUC |
| 31945 | | SfGn001fA*SfA*SfG*SmG*SmU*SmU*SmC*SmUn001mA* | AGGUUCUAAACCA | SSSSSSnXSSSSSnX | |
| | | SmA*SmA*SC*SC*SAn001mU*SmC*SmC*SmU | UCCU | SSS | |
| WV- | 650 | fCn001fUn001fCn001fU*SfU*SfU*SfC*SfC*SfA*SfU*SfG* | CUCUUUCCAUGGA | nXnXnXSSSSSSSSS | LUC |
| 31947 | | SfG*SfA*SfA*SfG*SmG*SmU*SmU*SmC*SmU*SmA*SmA* | AGGUUCUAAACCA | SSSSSSSSSSSSSSS | |
| | | SmA*SC*SC*SA*SmU*SmC*SmC*SmU | UCCU | SS | |
| WV- | 651 | fC*SfU*SfC*SfU*SfU*SfU*SfC*SfC*SfA*SfU* | CUCUUUCCAUGGA | SSSSSSSSSSnXnXn | LUC |
| 31948 | | SfGn001fGn001fAn001fA*SfG*SmG*SmU*SmU*SmC*SmU* | AGGUUCUAAACCA | XSSSSSSSSSSSSSS | |
| | | SmA*SmA*SmA*SC*SC*SA*SmU*SmC*SmC*SmU | UCCU | SS | |
| WV- | 652 | fC*SfU*SfC*SfU*SfU*SfU*SfC*SfC*SfA*SfU*SfG*SfG* | CUCUUUCCAUGGA | SSSSSSSSSSSSSSS | LUC |
| 31950 | | SfA*SfA*SfG*SmG*SmU*SmU*SmCn001mUn001mAn001mA* | AGGUUCUAAACCA | SSSnXnXnXSSSSSS | |
| | | SmA*SC*SC*SA*SmU*SmC*SmC*SmU | UCCU | SS | |
| WV- | 653 | fC*SfU*SfC*SfU*SfU*SfU*SfC*SfC*SfA*SfU*SfG*SfG* | CUCUUUCCAUGGA | SSSSSSSSSSSSSSS | LUC |
| 31952 | | SfA*SfA*SfG*SmG*SmU*SmU*SmC*SmU*SmA*SmA*SmA* | AGGUUCUAAACCA | SSSSSSSSSSnXnXn | |
| | | SC*SC*SAn001mUn001mCn001mCn001mU | UCCU | XnX | |
| WV- | 654 | fC*SfU*SfC*SfU*SfU*SfU*SfC*SfC*SfA*SfU*SfG*SfG* | CUCUUUCCAUGGA | SSSSSSSSSSSSSSS | LUC |
| 31953 | | SfA*SfA*SfG*SmG*SmU*SmU*SmC*SmU*SmA*SmA*SmA* | AGGUUCUAAACCA | SSSSSSSSSSSnXnX | |
| | | SC*SC*SA*SmUn001mCn001mCn001mU | UCCU | nX | |
| WV- | 655 | fC*SfU*SfC*SfU*SfU*SfU*SfC*SfC*SfA*SfU*SfG*SfG* | CUCUUUCCAUGGA | SSSSSSSSSSSSSSS | LUC |
| 31954 | | SfA*SfA*SfG*SmG*SmU*SmU*SmC*SmU*SmA*SmA*SmA* | AGGUUCUAAACCA | SSSSSSSSSSnRSSS | |
| | | SC*SC*SAn001RmU*SmC*SmC*SmU | UCCU | | |
| WV- | 656 | fC*SfU*SfC*SfU*SfU*SfU*SfC*SfC*SfA*SfU*SfG*SfG* | CUCUUUCCAUGGA | SSSSSSSSSSSSSSS | LUC |
| 31955 | | SfA*SfA*SfG*SmG*SmU*SmU*SmC*SmU*SmA*SmA*SmA* | AGGUUCUAAACCA | SSSSSSSSSSnSSSS | |
| | | SC*SC*SAn001SmU*SmC*SmC*SmU | UCCU | | |
| WV- | 657 | fC*SfU*SfCn001RfU*SfU*SfU*SfC*SfC*SfA*SfU*SfG* | CUCUUUCCAUGGA | SSnRSSSSSSSSSSS | LUC |
| 31956 | | SfG*SfA*SfA*SfG*SmG*SmU*SmU*SmC*SmU*SmA*SmA*S | AGGUUCUAAACCA | SSSSSSSSSSSSSSS | |
| | | mA*SC*SC*SA*SmU*SmC*SmC*SmU | UCCU | | |
| WV- | 658 | fC*SfU*SfC*SfU*SfU*SfU*SfC*SfC*SfA*SfU*SfG* | CUCUUUCCAUGGA | SSSSSSSSSSSnRSS | LUC |
| 31958 | | SfGn001RfA*SfA*SfG*SmG*SmU*SmU*SmC*SmU*SmA*SmA* | AGGUUCUAAACCA | SSSSSSSSSSSSSSS | |
| | | SmA*SC*SC*SA*SmU*SmC*SmC*SmU | UCCU | | |
| WV- | 659 | fC*SfU*SfC*SfU*SfU*SfU*SfC*SfC*SfA*SfU*SfG* | CUCUUUCCAUGGA | SSSSSSSSSSSnSSS | LUC |
| 31959 | | SfGn001SfA*SfA*SfG*SmG*SmU*SmU*SmC*SmU*SmA*SmA* | AGGUUCUAAACCA | SSSSSSSSSSSSSSS | |
| | | SmA*SC*SC*SA*SmU*SmC*SmC*SmU | UCCU | | |
| WV- | 660 | fC*SfU*SfC*SfU*SfU*SfU*SfC*SfC*SfA*SfU*SfG*SfG* | CUCUUUCCAUGGA | SSSSSSSSSSSSSSS | LUC |
| 31960 | | SfA*SfA*SfG*SmG*SmU*SmU*SmC*SmUn001RmA*SmA* | AGGUUCUAAACCA | SSSSnRSSSSSSSSS | |
| | | SmA*SC*SC*SA*SmU*SmC*SmC*SmU | UCCU | | |
| WV- | 661 | fC*SfU*SfC*SfU*SfU*SfU*SfC*SfC*SfA*SfU*SfG*SfG* | CUCUUUCCAUGGA | SSSSSSSSSSSSSSS | LUC |
| 31961 | | SfA*SfA*SfG*SmG*SmU*SmU*SmC*SmUn001SmA*SmA* | AGGUUCUAAACCA | SSSSnSSSSSSSSSS | |
| | | SmA*SC*SC*SA*SmU*SmC*SmC*SmU | UCCU | | |
| WV- | 662 | fC*SfU*SfC*SfU*SfU*SfU*SfC*SfC*SfA*SfU*SfG*SfG* | CUCUUUCCAUGGA | SSSSSSSSSSSSSSS | LUC |
| 31962 | | SfA*SfA*SfG*SmG*SmU*SmU*SmC*SmU*SmA*SmA*SmA* | AGGUUCUAAACCA | SSSSSSSSSSSSSnR | |
| | | SC*SC*SA*SmU*SmC*SmCn001RmU | UCCU | | |
| WV- | 663 | fC*SfU*SfC*SfU*SfU*SfU*SfC*SfC*SfA*SfU*SfG*SfG* | CUCUUUCCAUGGA | SSSSSSSSSSSSSSS | LUC |
| 31963 | | SfA*SfA*SfG*SmG*SmU*SmU*SmC*SmU*SmA*SmA*SmA* | AGGUUCUAAACCA | SSSSSSSSSSSSSnS | |
| | | SC*SC*SA*SmU*SmC*SmCn001SmU | UCCU | | |
| WV- | 664 | Mod001L001fA*SfC*SfA*SfU*SfA*SfA*SfU*SfU*SfU* | ACAUAAUUUACAC | OSSSSSSSSSSSSSS | ACTB |
| 32094 | | SfA*SfC*SfA*SfC*SfA*SfA*SmA*SmG*SmG*SmC*SmA* | AAAGGCAAUGCCA | SSSSSSSSSSSSSSS | |
| | | SmA*SmU*SmG*SC*SC*SA*SmU*SmC*SmA*SmC | UCAC | | |
| WV- | 665 | Mod001L001fA*SfC*SfA*SfU*SfA*SfA*SfU*SfU*SfU* | ACAUAAUUUACAC | OSSSSSSSSSSSSSS | ACTB |
| 32095 | | SfA*SfC*SfA*SfC*SfA*SfA*SmA*SmG*SmG*SmC*SmA* | AAAGGCAAUGCCA | SSSSSSSSSSSSSSS | |
| | | SmA*SmU*SmG*SfC*SC*SA*SmU*SmC*SmA*SmC | UCAC | | |
| WV- | 666 | Mod001L001fA*SfC*SfA*SfU*SfA*SfA*SfU*SfU*SfU* | ACAUAAUUUACAC | OSSSSSSSSSSSSSS | ACTB |
| 32096 | | SfA*SfC*SfA*SfC*SfA*SfA*SmA*SmG*SmG*SmC*SmA* | AAAGGCAAUGCCA | SSSSSSSSSSRRSSS | |
| | | SmA*SmU*SmG*SC*SC*RA*RmU*SmC*SmA*SmC | UCAC | | |
| WV- | 667 | Mod001L001fA*SfC*SfA*SfU*SfA*SfA*SfU*SfU*SfU* | ACAUAAUUUACAC | OSSSSSSSSSSSSSS | ACTB |
| 32097 | | SfA*SfC*SfA*SfC*SfA*SfA*SmA*SmG*SmG*SmC*SmA* | AAAGGCAAUGCCA | SSSSSSSSSSRRSSS | |
| | | SmA*SmU*SmG*SfC*SC*RA*RmU*SmC*SmA*SmC | UCAC | | |
| WV- | 668 | Mod001L001fA*SfC*SfA*SfU*SfA*SfA*SfU*SfU*SfU* | ACAUAAUUUACAC | OSSSSSSSSSSSSSS | ACTB |
| 32098 | | SfA*SfC*SfA*SfC*SfA*SfA*SmA*SmG*SmG*SmC*SmA* | AAAGGCAAUGCCA | SSSSSSSSSSSnXSS | |
| | | SmA*SmU*SmG*SC*SC*SAn001mU*SmC*SmA*SmC | UCAC | S | |
| WV- | 669 | Mod001L001fA*SfC*SfA*SfU*SfA*SfA*SfU*SfU*SfU* | ACAUAAUUUACAC | OSSSSSSSSSSSSSS | ACTB |
| 32099 | | SfA*SfC*SfA*SfC*SfA*SfA*SmA*SmG*SmG*SmC*SmA* | AAAGGCAAUGCCA | SSSSSSSSSSSSSSn | |
| | | SmA*SmU*SmG*SC*SC*SA*SmU*SmC*SmAn001mC | UCAC | X | |
| WV- | 670 | Mod001L001fA*SfC*SfA*SfU*SfA*SfA*SfU*SfU*SfU* | ACAUAAUUUACAC | OSSSSSSSSSSSSSS | ACTB |
| 32100 | | SfA*SfC*SfA*SfC*SfA*SfA*SmA*SmG*SmG*SmC*SmA* | AAAGGCAAUGCCA | SSSSSSSSSSSnXSS | |
| | | SmA*SmU*SmG*SC*SC*SAn001mU*SmC*SmAn001mC | UCAC | nX | |
| WV- | 671 | Mod001L001fA*SfC*SfA*SfU*SfA*SfA*SfU*SfU*SfU* | ACAUAAUUUACAC | OSSSSSSSSSSSSSS | ACTB |
| 32101 | | SfA*SfC*SfA*SfC*SfA*SfA*SmA*SmG*SmG*SmC* | AAAGGCAAUGCCA | SSSSSnXSSSSSnXS | |
| | | SmAn001mA*SmU*SmG*SC*SC*SAn001mU*SmC*SmAn001mC | UCAC | SnX | |
| WV- | 672 | Mod001L001fU*SfA*SfC*SfA*SfU*SfA*SfA*SfU*SfU* | UACAUAAUUUACA | OSSSSSSSSSSSSSS | ACTB |
| 32102 | | SfU*SfA*SfC*SfA*SfC*SfA*SfA*SmA*SmG*SmG*SmC*SmA* | CAAAGGCAAUGCC | SSSSSSSSSSSSSSS | |
| | | SmA*SmU*SmG*SC*SC*SA*SmU*SmC*SmA*SmC*SmC | AUCACC | SS | |
| WV- | 673 | Mod001L001fA*fC*fA*fU*fA*fA*fU*fU*fU*fA*fC*fA*fC* | ACAUAAUUUACAC | OXXXXXXXXXXX | ACTB |
| 32103 | | fA*fA*mA*mG*mG*mC*mA*mA*mU*mG*C*C*A*mU*mC* | AAAGGCAAUGCCA | XXXXXXXXXXXX | |
| | | mA*mC | UCAC | XXXXXX | |
| WV- | 674 | Mod001L001fAn001fC*SfA*SfU*SfA*SfA*SfU*SfU*SfU* | ACAUAAUUUACAC | OnXSSSSSSSSSSSS | ACTB |
| 35712 | | SfA*SfC*SfA*SfC*SfA*SfA*SmA*SmG*SmG*SmC* | AAAGGCAAUGCCA | SSSSSSnXSSSSSnX | |
| | | SmAn001mA*SmU*SmG*SC*SC*SAn001mU*SmC*SmAn001mC | UCAC | SSnX | |
| WV- | 675 | Mod001L001fAn001fC*SfA*SfU*SfA*SfA*SfU*SfU*SfU* | ACAUAAUUUACAC | OnXSSSSSSSSSSSS | ACTB |
| 35713 | | SfA*SfC*SfA*SfC*SfAn001fA*SmAn001mG*SmG*SmC*SmA* | AAAGGCAAUGCCA | nXSnXSSSSSSSSSn | |
| | | SmA*SmU*SmG*SC*SC*SAn001mU*SmC*SmAn001mC | UCAC | XSSnX | |
| WV- | 676 | Mod001L001fAn001fC*SfA*SfU*SfA*SfA*SfU*SfU*SfU* | ACAUAAUUUACAC | OnXSSSSSSSSSnXS | ACTB |
| 35714 | | SfA*SfCn001fA*SfC*SfAn001fA*SmA*SmG*SmG*SmC*SmA* | AAAGGCAAUGCCA | SnXSSSSSSSSSSSn | |
| | | SmA*SmU*SmG*SC*SC*SAn001mU*SmC*SmAn001mC | UCAC | XSSnX | |
| WV- | 677 | Mod001L001fA*SfC*SfA*SfU*SfA*SfA*SfU*SfU*SfU*SfA* | ACAUAAUUUACAC | OSSSSSSSSSSSSSS | ACTB |
| 35715 | | SfC*SfA*SfC*SfA*SfA*SmA*SmG*SmG*SmC*SmAn001mA* | AAAGGCAAUGCCA | SSSSSnXSSSSRnXS | |
| | | SmU*SmG*SC*SC*RAn001mU*SmC*SmAn001mC | UCAC | SnX | |
| WV- | 678 | Mod001L001fAn001fC*SfA*SfU*SfA*SfA*SfU*SfU*SfU* | ACAUAAUUUACAC | OnXSSSSSSSSSSSS | ACTB |
| 35716 | | SfA*SfC*SfA*SfC*SfA*SfA*SmA*SmG*SmG*SmC* | AAAGGCAAUGCCA | SSSSSSnXSSSSRnX | |
| | | SmAn001mA*SmU*SmG*SC*SC*RAn001mU*SmC*SmAn001mC | UCAC | SSnX | |
| WV- | 679 | Mod001L001fAn001fC*SfA*SfU*SfA*SfA*SfU*SfU*SfU* | ACAUAAUUUACAC | OnXSSSSSSSSSSSS | ACTB |
| 35717 | | SfA*SfC*SfA*SfC*SfAn001fA*SmAn001mG*SmG*SmC* | AAAGGCAAUGCCA | nXSnXSSSSSSSSRn | |
| | | SmA*SmA*SmU*SmG*SC*SC*RAn001mU*SmC*SmAn001mC | UCAC | XSSnX | |
| WV- | 680 | Mod001L001fAn001fC*SfA*SfU*SfA*SfA*SfU*SfU*SfU* | ACAUAAUUUACAC | OnXSSSSSSSSSnXS | ACTB |
| 35718 | | SfA*SfCn001fA*SfC*SfAn001fA*SmA*SmG*SmG*SmC* | AAAGGCAAUGCCA | SnXSSSSSSSSSSRn | |
| | | SmA*SmA*SmU*SmG*SC*SC*RAn001mU*SmC*SmAn001mC | UCAC | XSSnX | |
| WV- | 681 | Mod001L001fA*SfC*SfA*SfU*SfA*SfA*SfU*SfU*SfU* | ACAUAAUUUACAC | OSSSSSSSSSSSSSS | ACTB |
| 35719 | | SfA*SfC*SfA*SfC*SfA*SfA*SmA*SmG*SmG*SmC* | AAAGGCAAUGCCA | SSSSSnXSSSSSnXS | |
| | | SmAn001mA*SmU*SmG*SfC*SC*SAn001mU*SmC*SmAn001mC | UCAC | SnX | |
| WV- | 682 | Mod001L001fAn001fC*SfA*SfU*SfA*SfA*SfU*SfU*SfU* | ACAUAAUUUACAC | OnXSSSSSSSSSSSS | ACTB |
| 35720 | | SfA*SfC*SfA*SfC*SfA*SfA*SmA*SmG*SmG*SmC* | AAAGGCAAUGCCA | SSSSSSnXSSSSSnX | |
| | | SmAn001mA*SmU*SmG*SfC*SC*SAn001mU*SmC*SmAn001mC | UCAC | SSnX | |
| WV- | 683 | Mod001L001fAn001fC*SfA*SfU*SfA*SfA*SfU*SfU*SfU* | ACAUAAUUUACAC | OnXSSSSSSSSSSSS | ACTB |
| 35721 | | SfA*SfC*SfA*SfC*SfAn001fA*SmAn001mG*SmG*SmC*SmA* | AAAGGCAAUGCCA | nXSnXSSSSSSSSSn | |
| | | SmA*SmU*SmG*SfC*SC*SAn001mU*SmC*SmAn001mC | UCAC | XSSnX | |
| WV- | 684 | Mod001L001fAn001fC*SfA*SfU*SfA*SfA*SfU*SfU*SfU* | ACAUAAUUUACAC | OnXSSSSSSSSSnXS | ACTB |
| 35722 | | SfA*SfCn001fA*SfC*SfAn001fA*SmA*SmG*SmG*SmC*SmA* | AAAGGCAAUGCCA | SnXSSSSSSSSSSSn | |
| | | SmA*SmU*SmG*SfC*SC*SAn001mU*SmC*SmAn001mC | UCAC | XSSnX | |
| WV- | 685 | Mod001L001fA*SfC*SfA*SfU*SfA*SfA*SfU*SfU*SfU*SfA* | ACAUAAUUUACAC | OSSSSSSSSSSSSSS | ACTB |
| 35723 | | SfC*SfA*SfC*SfA*SfA*SmA*SmG*SmG*SmC*SmAn001mA* | AAAGGCAAUGCCA | SSSSSnXSSSSRnXS | |
| | | SmU*SmG*SfC*SC*RAn001mU*SmC*SmAn001mC | UCAC | SnX | |
| WV- | 686 | Mod001L001fAn001fC*SfA*SfU*SfA*SfA*SfU*SfU*SfU* | ACAUAAUUUACAC | OnXSSSSSSSSSSSS | ACTB |
| 35724 | | SfA*SfC*SfA*SfC*SfA*SfA*SmA*SmG*SmG*SmC*SmAn001mA* | AAAGGCAAUGCCA | SSSSSSnXSSSSRnX | |
| | | SmU*SmG*SfC*SC*RAn001mU*SmC*SmAn001mC | UCAC | SSnX | |
| WV- | 687 | Mod001L001fAn001fC*SfA*SfU*SfA*SfA*SfU*SfU*SfU* | ACAUAAUUUACAC | OnXSSSSSSSSSSSS | ACTB |
| 35725 | | SfA*SfC*SfA*SfC*SfAn001fA*SmAn001mG*SmG*SmC*SmA* | AAAGGCAAUGCCA | nXSnXSSSSSSSSRn | |
| | | SmA*SmU*SmG*SfC*SC*RAn001mU*SmC*SmAn001mC | UCAC | XSSnX | |
| WV- | 688 | Mod001L001fAn001fC*SfA*SfU*SfA*SfA*SfU*SfU*SfU* | ACAUAAUUUACAC | OnXSSSSSSSSSnXS | ACTB |
| 35726 | | SfA*SfCn001fA*SfC*SfAn001fA*SmA*SmG*SmG*SmC*SmA* | AAAGGCAAUGCCA | SnXSSSSSSSSSSRn | |
| | | SmA*SmU*SmG*SfC*SC*RAn001mU*SmC*SmAn001mC | UCAC | XSSnX | |
| WV- | 689 | Mod001L001fA*SfC*SfA*SfU*SfA*SfA*SfU*SfU*SfU*SfA* | ACAUAAUUUACAC | OSSSSSSSSSSSSSS | ACTB |
| 35735 | | SfC*SfA*SfC*SfG*SfA*SmA*SmA*SmG*SmC*SmAn001mA* | GAAAGCAAUGCCA | SSSSSnXSSSSSnXS | |
| | | SmU*SmG*SfC*SC*SAn001mU*SmC*SmAn001mC | UCAC | SnX | |
| WV- | 690 | Mod001L001fAn001fC*SfA*SfU*SfA*SfA*SfU*SfU*SfU* | ACAUAAUUUACAC | OnXSSSSSSSSSSSS | ACTB |
| 35736 | | SfA*SfC*SfA*SfC*SfG*SfA*SmA*SmA*SmG*SmC*SmAn001mA* | GAAAGCAAUGCCA | SSSSSSnXSSSSSnX | |
| | | SmU*SmG*SfC*SC*SAn001mU*SmC*SmAn001mC | UCAC | SSnX | |
| WV- | 691 | Mod001L001fAn001fC*SfA*SfU*SfA*SfA*SfU*SfU*SfU* | ACAUAAUUUACAC | OnXSSSSSSSSSSSS | ACTB |
| 35737 | | SfA*SfC*SfA*SfC*SfGn001fA*SmAn001mA*SmG*SmC*SmA* | GAAAGCAAUGCCA | nXSnXSSSSSSSSSn | |
| | | SmA*SmU*SmG*SfC*SC*SAn001mU*SmC*SmAn001mC | UCAC | XSSnX | |
| WV- | 692 | Mod001L001fAn001fC*SfA*SfU*SfA*SfA*SfU*SfU*SfU* | ACAUAAUUUACAC | OnXSSSSSSSSSnXS | ACTB |
| 35738 | | SfA*SfCn001fA*SfC*SfGn001fA*SmA*SmA*SmG*SmC*SmA* | GAAAGCAAUGCCA | SnXSSSSSSSSSSSn | |
| | | SmA*SmU*SmG*SfC*SC*SAn001mU*SmC*SmAn001mC | UCAC | XSSnX | |
| WV- | 693 | Mod001L001fA*SfC*SfA*SfU*SfA*SfA*SfU*SfU*SfU*SfA* | ACAUAAUUUACAC | OSSSSSSSSSSSSSS | ACTB |
| 35739 | | SfC*SfA*SfC*SfG*SfA*SmA*SmA*SmG*SmC*SmAn001mA* | GAAAGCAAUGCCA | SSSSSnXSSSSRnXS | |
| | | SmU*SmG*SfC*SC*RAn001mU*SmC*SmAn001mC | UCAC | SnX | |
| WV- | 694 | Mod001L001fAn001fC*SfA*SfU*SfA*SfA*SfU*SfU*SfU* | ACAUAAUUUACAC | OnXSSSSSSSSSSSS | ACTB |
| 35740 | | SfA*SfC*SfA*SfC*SfG*SfA*SmA*SmA*SmG*SmC*SmAn001mA* | GAAAGCAAUGCCA | SSSSSSnXSSSSRnX | |
| | | SmU*SmG*SfC*SC*RAn001mU*SmC*SmAn001mC | UCAC | SSnX | |
| WV- | 695 | Mod001L001fAn001fC*SfA*SfU*SfA*SfA*SfU*SfU*SfU* | ACAUAAUUUACAC | OnXSSSSSSSSSSSS | ACTB |
| 35741 | | SfA*SfC*SfA*SfC*SfGn001fA*SmAn001mA*SmG*SmC*SmA* | GAAAGCAAUGCCA | nXSnXSSSSSSSSRn | |
| | | SmA*SmU*SmG*SfC*SC*RAn001mU*SmC*SmAn001mC | UCAC | XSSnX | |
| WV- | 696 | Mod001L001fAn001fC*SfA*SfU*SfA*SfA*SfU*SfU*SfU* | ACAUAAUUUACAC | OnXSSSSSSSSSnXS | ACTB |
| 35742 | | SfA*SfCn001fA*SfC*SfGn001fA*SmA*SmA*SmG*SmC*SmA* | GAAAGCAAUGCCA | SnXSSSSSSSSSSRn | |
| | | SmA*SmU*SmG*SfC*SC*RAn001mU*SmC*SmAn001mC | UCAC | XSSnX | |

TABLE 1E
Example oligonucleotides and/or compositions.

| | SEQ | | | Stereochemistry / | |
|---|---|---|---|---|---|
| ID | ID NO | Description | Base Sequence | Linkage | Target |
| WV- | 697 | fA*SfC*SfA*SfU*SfA*SfA*SfU*SfU*SfU*SfA*SfC*SfA*SfC* | ACAUAAUUUACAC | SSSSSSSSSSSSSSS | ACTB |
| 27404 | | SfG*SfA*SmA*SmA*SmG*SmC*SmA*SmA*SmU*SmG* | GAAAGCAAUGCCA | SSSSSSSSSSSSSS | |
| | | SfC*SC*SA*SmU*SmC*SmA*SmC | UCAC | | |
| WV- | 698 | Mod001L001fA*fC*fA*fU*fA*fA*fU*fU*fU*fA*fG*fA*fC* | ACAUAAUUUAGAC | OXXXXXXXXXXX | ACTB |
| 30298 | | fG*fU*mA*mA*mG*mC*mA*mA*mU*mG*fC*C*A*mU* | GUAAGCAAUGCCA | XXXXXXXXXXXX | |
| | | mC*mA*mC | UCAC | XXXXXX | |
| WV- | 699 | Mod001L001fAn001RfC*SfA*SfU*SfA*SfA*SfU*SfU*SfU* | ACAUAAUUUACAC | OnRSSSSSSSSSSSS | ACTB |
| 37314 | | SfA*SfC*SfA*SfC*SfGn001RfA*SmAn001RmA*SmG*SmC* | GAAAGCAAUGCCA | nRSnRSSSSSSSSSn | |
| | | SmA*SmA*SmU*SmG*SfC*SC*SAn001RmU*SmC*SmAn001RmC | UCAC | RSSnR | |
| WV- | 700 | Mod001L001fAn001SfC*SfA*SfU*SfA*SfA*SfU*SfU*SfU* | ACAUAAUUUACAC | OnSSSSSSSSSSSSS | ACTB |
| 37315 | | SfA*SfC*SfA*SfC*SfGn001SfA*SmAn001SmA*SmG*SmC* | GAAAGCAAUGCCA | nSSnSSSSSSSSSSnS | |
| | | SmA*SmA*SmU*SmG*SfC*SC*SAn001SmU*SmC*SmAn001SmC | UCAC | SSnS | |
| WV- | 701 | fAn001RfC*SfA*SfU*SfA*SfA*SfU*SfU*SfU*SfA*SfC*SfA* | ACAUAAUUUACAC | nRSSSSSSSSSSSSn | ACTB |
| 37317 | | SfC*SfGn001RfA*SmAn001RmA*SmG*SmC*SmA*SmA* | GAAAGCAAUGCCA | RSnRSSSSSSSSSnR | |
| | | SmU*SmG*SfC*SC*SAn001RmU*SmC*SmAn001RmC | UCAC | SSnR | |
| WV- | 702 | fAn001SfC*SfA*SfU*SfA*SfA*SfU*SfU*SfU*SfA*SfC*SfA* | ACAUAAUUUACAC | nSSSSSSSSSSSSSnS | ACTB |
| 37318 | | SfC*SfGn001SfA*SmAn001SmA*SmG*SmC*SmA*SmA* | GAAAGCAAUGCCA | SnSSSSSSSSSSnSSS | |
| | | SmU*SmG*SfC*SC*SAn001SmU*SmC*SmAn001SmC | UCAC | nS | |
| WV- | 703 | Aeon001Rm5Ceo*SAeo*SfU*SfA*SfA*SfU*SfU*SfU*SfA* | ACAUAAUUUACAC | nRSSSSSSSSSSSSn | ACTB |
| 37324 | | SfC*SfA*SfC*SfGn001RfA*SmAn001RmA*SmG*SmC*SmA* | GAAAGCAAUGCCA | RSnRSSSSSSSSSnR | |
| | | SmA*SmU*SmG*SfC*SC*SAn001RmU*SmC*SmAn001RmC | UCAC | SSnR | |
| WV- | 704 | Mod001L001Aeon001Rm5Ceo*SAeo*SfU*SfA*SfA*SfU*SfU* | ACAUAAUUUACAC | OnRSSSSSSSSSSSS | ACTB |
| 37330 | | SfU*SfA*SfC*SfA*SfC*SfGn001RfA*SmAn001RmA*SmG* | GAAAGCAAUGCCA | nRSnRSSSSSSSSSn | |
| | | SmC*SmA*SmA*SmU*SmG*SfC*SC*SAn001RmU*SmC* | UCAC | RSSnR | |
| | | SmAn001RmC | | | |
| WV- | 705 | Mod001L00lfC*SfC*SfA*SfA*SfC*SfC*SfA*SfG*SfA*SfA* | CCAACCAGAAAUU | OSSSSSSSSSSSSSS | EEF1A1 |
| 38697 | | SfA*SfU*SfU*SfG*SfG*SmC*SmA*SmC*SmA*SmA*SmA* | GGCACAAAUGCCA | SSSSSSSSSSRRSSS | |
| | | SmU*SmG*SfC*SC*RA*RmC*SmU*SmG*SmU | CUGU | | |
| WV- | 706 | Mod001L001fC*fC*fA*fA*fC*fC*fA*fG*fA*fA*fA*fU*fU* | CCAACCAGAAAUU | OXXXXXXXXXXX | EEF1A1 |
| 38698 | | fG*fG*mC*mA*mC*mA*mA*mA*mU*mG*fC*C*A*mC*mU* | GGCACAAAUGCCA | XXXXXXXXXXXX | |
| | | mG*mU | CUGU | XXXXXX | |
| WV- | 707 | Mod001L001fCn001RfC*SfA*SfA*SfC*SfC*SfA*SfG*SfA* | CCAACCAGAAAUU | OnRSSSSSSSSSSSS | EEF1A1 |
| 38699 | | SfA*SfA*SfU*SfU*SfGn001RfG*SmCn001RmA*SmC*SmA* | GGCACAAAUGCCA | nRSnRSSSSSSSSSn | |
| | | SmA*SmA*SmU*SmG*SfC*SC*SAn001RmC*SmU*SmGn001RmU | CUGU | RSSnR | |
| WV- | 708 | fCn001RfC*SfA*SfA*SfC*SfC*SfA*SfG*SfA*SfA*SfA*SfU* | CCAACCAGAAAUU | nRSSSSSSSSSSSSn | EEF1A1 |
| 40591 | | SfU*SfGn001RfG*SmCn001RmA*SmC*SmA*SmA*SmA* | GGCACAAAUGCCA | RSnRSSSSSSSSSnR | |
| | | SmU*SmG*SfC*SC*SAn001RmC*SmU*SmGn001RmU | CUGU | SSnR | |
| WV- | 709 | fCn001RfA*SfG*SfA*SfA*SfG*SfG*SfA*SfA*SfC*SfA*SfU* | CAGAAGGAACAUG | nRSSSSSSSSSSSSn | GHITM |
| 40594 | | SfG*SfCn001RfU*SmGn001RmA*SmA*SmA*SmA*SmG* | CUGAAAAGAACCA | RSnRSSSSSSSSSnR | |
| | | SmA*SmA*SfC*SC*SAn001RmA*SmU*SmCn001RmC | AUCC | SSnR | |
| WV- | 710 | fUn001RfG*SfC*SfC*SfC*SfU*SfG*SfA*SfA*SfU*SfU*SfC* | UGCCCUGAAUUCC | nRSSSSSSSSSSSSn | HSP90AB1 |
| 40595 | | SfC*SfAn001RfA*SmCn001RmU*SmG*SmA*SmC*SmC* | AACUGACCUUCCA | RSnRSSSSSSSSSnR | |
| | | SmU*SmU*SfC*SC*SAn001RmC*SmA*SmGn001RmA | CAGA | SSnR | |
| WV- | 711 | fUn001RfG*SfA*SfG*SfG*SfC*SfG*SfA*SfA*SfG*SfC*SfA* | UGAGGCGAAGCAU | nRSSSSSSSSSSSSn | HSP90B1 |
| 40596 | | SfU*SfUn001RfC*SmUn001RmU*SmU*SmC*SmU*SmA* | UCUUUCUAUUCCA | RSnRSSSSSSSSSnR | |
| | | SmU*SmU*SfC*SC*SAn001RmU*SmC*SmUn001RmC | UCUC | SSnR | |
| WV- | 712 | mUmCmUrCrUrUrUrCrCrArUrGrGrArArGrGrUrUrCrUrArAr | UCUCUUUCCAUGG | OOOOOOOOOOOO | UUC |
| 20705 | | ArCrCrArUrCmCmU*mG | AAGGUUCUAAACC | OOOOOOOOOOOO | |
| | | | AUCCUG | OOOOOOX | |
| WV- | 713 | fUn001RfU*SfA*SfA*SfU*SfC*SfC*SfA*SfU*SfC*SfU*SfC* | UUAAUCCAUCUCU | nRSSSSSSSSSSSSn | SRSF1 |
| 40592 | | SfU*SfUn001RfC*SmAn001RmG*SmA*SmU*SmA*SmU* | UCAGAUAUGUCCA | RSnRSSSSSSSSSnR | |
| | | SmG*SmU*SfC*SC*SAn001RmC*SmA*SmGn001RmA | CAGA | SSnR | |
| WV- | 714 | Mod001U001fA*SfU*SfC*SfC*SfA*SfC*SfU*SfG*SfU*SfG* | AUCCACUGUGGCA | OSSSSSSSSSSSSSS | UGP2 |
| 38700 | | SfG*SfC*SfA*SfC*SfC*SmC*SmA*SmG*SmA*SmU*SmU* | CCCAGAUUAUCCA | SSSSSSSSSSRRSSS | |
| | | SmA*SmU*SfC*SC*RA*RmU*SmG*SmU*SmU | UGUU | | |
| WV- | 715 | Mod001L001fA*fU*fC*fC*fA*fC*fU*fG*fU*fG*fG*fC*fA* | AUCCACUGUGGCA | OXXXXXXXXXXX | UGP2 |
| 38701 | | fC*fC*mC*mA*mG*mA*mU*mU*mA*mU*fC*C*A*mU*mG* | CCCAGAUUAUCCA | XXXXXXXXXXXX | |
| | | mU*mU | UGUU | XXXXXX | |
| WV- | 716 | Mod001U001fAn001RfU*SfC*SfC*SfA*SfC*SfU*SfG*SfU* | AUCCACUGUGGCA | OnRSSSSSSSSSSSS | UGP2 |
| 38702 | | SfG*SfG*SfC*SfA*SfCn001RfC*SmCn001RmA*SmG*SmA* | CCCAGAUUAUCCA | nRSnRSSSSSSSSSn | |
| | | SmU*SmU*SmA*SmU*SfC*SC*SAn001RmU*SmG*SmUn001RmU | UGUU | RSSnR | |
| WV- | 717 | fAn001RfU*SfC*SfC*SfA*SfC*SfU*SfG*SfU*SfG*SfG*SfC* | AUCCACUGUGGCA | nRSSSSSSSSSSSSn | UGP2 |
| 40590 | | SfA*SfCn001RfC*SmCn001RmA*SmG*SmA*SmU*SmU* | CCCAGAUUAUCCA | RSnRSSSSSSSSSnR | |
| | | SmA*SmU*SfC*SC*SAn001RmU*SmG*SmUn001RmU | UGUU | SSnR | |

TABLE 1F
Example oligonucleotides and/or compositions that target ACTB.

| | SEQ | | | Stereochemistry/ |
|---|---|---|---|---|
| ID | ID NO | Description | Base Sequence | Linkage |
| WV- | 718 | fAn001RfC*SfA*SfU*SA*SA*SfU*SU*SfU*SfA*SfC-SA*SfC* | ACAUAAUUUACA | nRSSSSSSSSSSSSnRSn |
| 37317 | | Sfn001RfA*SmAn001RmA*SmG*SmC*SmA*SmA*SmU*SmG*SfC* | CGAAAGCAAUGC | RSSSSSSSSSnRSSR |
| | | SC*SAn001RmU*SmC*SmAn001RmC | CAUCAC | |
| WV- | 719 | fAn001SfC*SfA*SfU*SfA*SfA*SfU*SfU*SfU*SfA*SfC*SfA*SfC* | ACAUAAUUUACA | nSSSSSSSSSSSSSnSSn |
| 37318 | | SfGn001SfA*SmAn001SmA*SmG*SmC*SmA*SmA*SmU*SmG*SfC* | CGAAAGCAAUGC | SSSSSSSSSSnSSSnS |
| | | SC*SAn001SmU*SmC*SmAn001SmC | CAUCAC | |

TABLE 1G
Example oligonucleotides and/or compositions that target LUC

| | SEQ | | | Stereochemistry/ |
|---|---|---|---|---|
| ID | ID NO | Description | Base Sequence | Linkage |
| WV- | 720 | fU*fC*AU*fC*fU*fU*fU*C*C*A*fU*fG*fG*fA*fA*GG*mG* | UCUCUUUCCAUG | XXXXXXXXXXXXXXXX |
| 23868 | | mU*mU*mC*mU*mA*mA*mA*C*C*A*mU*mC*mC*mU*mG | GAAGGUUCUAAA | XXXXXXXXXXXXXXX |
| | | | CCAUCCUG | |
| WV- | 721 | fC*fU*fC*fU*fU*fU*fC*fC*fA*fU*fG*fG*fA*fA*G*mG*mU* | CUCUUUCCAUGG | XXXXXXXXXXXXXXXX |
| 23872 | | mU*mC*mU*mA*mA*mA*C*C*A*mU*mC*mC*mU*mG | AAGGUUCUAAAC | XXXXXXXXXXXXXX |
| | | | CAUCCUG | |
| WV- | 722 | fC*fU*fC*fU*fU*fU*fC*fC*fA*fU*fG*fG*fA*fA*fG*mG*mU* | CUCUUUCCAUGG | XXXXXXXXXXXXXXXX |
| 23873 | | mU*mC*mU*mA*mA*mA*C*C*A*mU*mC*mC*mU | AAGGUUCUAAAC | XXXXXXXXXXXXX |
| | | | CAUCCU | |
| WV- | 723 | fC*SfU*SfC*SfU*SfU*SfU*SC*SfC*SfA*SfU*SfG*SfG*SfA* | CUCUUUCCAUGG | SSSSSSSSSSSSSSSSSSSSS |
| 24111 | | SfA*SfG*SmG*SmU*SmU*SmC*SmU*SmA*SmA*SmA*SC* | AAGGUUCUAAAC | SSSSSSSS |
| | | SC*SA*SmU*SmC*SmC*SmU | CAUCCU | |
| WV- | 724 | fC*fU*fC*fU*fU*fU*fC*fC*A*fU*fG*fG*fA*fA*fG*mG*mU* | CUCUUUCCAUGG | XXXXXXXXXXXXXXXX |
| 27520 | | mU*mC*mU*mA*mA*mA*C*C*A*mU*mC*mC*mU | AAGGUUCUAAAC | XXXXXXXXXXXXX |
| | | | CAUCCU | |
| WV- | 725 | fC*fU*fC*fU*fU*fU*fC*fC*fA*fU*fG*fG*fA*fA*fG*mG*mU* | CUCUUUCCAUGG | XXXXXXXXXXXXXXXX |
| 27521 | | mU*mC*mU*mA*mA*mA*C*U*A*mU*mC*mC*mU | AAGGUUCUAAAC | XXXXXXXXXXXXX |
| | | | UAUCCU | |
| WV- | 726 | fC*fU*fC*fU*fU*fU*AC*fC*fA*fU*fG*fG*fA*fA*fG*mG*mU* | CUCUUUCCAUGG | XXXXXXXXXXXXXXXX |
| 27522 | | mU*mC*mU*mA*mA*mA*CT*A*mU*mC*mC*mU | AAGGUUCUAAAC | XXXXXXXXXXXXX |
| | | | TAUCCU | |
| WV- | 727 | fC*fU*fC*AU*fU*fU*fC*fC*fA*fU*fG*fG*fA*fA*G*mG*mU* | CUCUUUCCAUGG | XXXXXXXXXXXXXXXX |
| 27523 | | mU*mC*mU*mA*mA*mA*C*zdnp*A*mU*mC*mC*mU | AAGGUUCUAAAC | XXXXXXXXXXXXX |
| | | | ZAUCCU | |
| WV- | 728 | fC*fU*fC*AU*fU*fU*fC*fC*fA*fU*fG*fG*A*fA*fG*mG*mU* | CUCUUUCCAUGG | XXXXXXXXXXXXXXXX |
| 27524 | | mU*mC*mU*mA*mA*mA*fC*zdnp*A*mU*mC*mC*mU | AAGGUUCUAAAC | XXXXXXXXXXXXX |
| | | | ZAUCCU | |
| WV- | 729 | fU*fC*fU*fC*fU*fU*fU*fC*fC*A*fU*fG*fG*fA*CA*fG*nG* | UCUCUUUCCAUG | XXXXXXXXXXXXXXXX |
| 31138 | | mU*mU*mC*mU*mA*mA*mA*C*b001A*A*mU*mC*mC*mU | GAAGGUUCUAAA | XXXXXXXXXXXXX |
| | | *mG | CAAUCCUG | |
| WV- | 730 | fC*fU*fC*fU*fU*fU*fC*fC*fA*fU*fG*fG*fA*fA*fG*mG*mU* | CUCUUUCCAUGG | XXXXXXXXXXXXXXXX |
| 31144 | | mU*mC*mU*m A*mA*mA*C*b001A*A*mU*mC*mC*mU | AAGGUUCUAAAC | XXXXXXXXXXXXX |
| | | | AAUCCU | |
| WV- | 731 | fU*fC*fU*fC*fU*fU*fU*fC*fC*fA*fU*fG*fG*fA*fA*fG*mG* | UCUCUUUCCAUG | XXXXXXXXXXXXXXXX |
| 34970 | | mU*mU*mC*mU*mA*mA*mA*C*b004U*A*mU*mC*mC*mU* | GAAGGUUCUAAA | XXXXXXXXXXXXXXX |
| | | mG | CUAUCCUG | |
| WV- | 732 | fU*fC*U*C*fU*fU*fU*fC*fC*fA*fU*fG*G*A*A*G*mG* | UCUCUUUCCAUG | XXXXXXXXXXXXXXXX |
| 34971 | | mU*mU*mC*mU*mA*mA*mA*C*b008U*A*mU*mC*mC*mU* | GAAGGUUCUAAA | XXXXXXXXXXXXXXX |
| | | mG | CUAUCCUG | |
| WV- | 733 | fU*fC*U*C*fU*fU*fU*fC*fC*fA*fU*fG*fG*fA*fA*fG*mG* | UCUCUUUCCAUG | XXXXXXXXXXXXXXXX |
| 34972 | | mU*mU*mC*mU*mA*mA*mA*C*b009U*A*mU*mC*mC*mU* | GAAGGUUCUAAA | XXXXXXXXXXXXXXX |
| | | mG | CUAUCCUG | |
| WV- | 734 | fU*fC*fU*fC*fU*fU*fU*fC*fC*fA*fU*fG*fG*fA*fA*fG*mG* | UCUCUUUCCAUG | XXXXXXXXXXXXXXXX |
| 34973 | | mU*mU*mC*mU*mA*mA*mA*C*A*A*mU*mC*mC*mU*mG | GAAGGUUCUAAA | XXXXXXXXXXXXXXX |
| | | | CAAUCCUG | |
| WV- | 735 | fU*fC*fU*fC*fU*fU*fU*fC*fC*fA*fU*fG*fG*fA*fA*fG*mG* | UCUCUUUCCAUG | XXXXXXXXXXXXXXXX |
| 34974 | | mU*mU*mC*mU*mA*mA*mA*C*L010*A*mU*mC*mC*mU* | GAAGGUUCUAAA | XXXXXXXXXXXXXXX |
| | | mG | CAUCCUG | |
| WV- | 736 | fC*fU*fC*fU*fU*fU*fC*fC*fA*fU*fG*AG*fA*fA*fG*mG*mU* | CUCUUUCCAUGG | XXXXXXXXXXXXXXXX |
| 34975 | | mU*mC*mU*mA*mA*mA*C*b004U*A*mU*mC*mC*mU | AAGGUUCUAAAC | XXXXXXXXXXXXX |
| | | | UAUCCU | |
| WV- | 737 | fC*fU*C*fU*fU*fU*fC*fC*fA*fU*fG*fG*fA*fA*fG*mG*mU* | CUCUUUCCAUGG | XXXXXXXXXXXXXXXX |
| 34976 | | mU*mC*mU*mA*mA*mA*C*b008U*A*mU*mC*mC*mU | AAGGUUCUAAAC | XXXXXXXXXXXXX |
| | | | UAUCCU | |
| WV- | 738 | fC*fU*fC*fU*fU*fU*fC*fC*fA*fU*fG*fG*fA*fA*fG*mG*mU* | CUCUUUCCAUGG | XXXXXXXXXXXXXXXX |
| 34977 | | mU*mC*mU*mA*mA*mA*C*b009U*A*mU*mC*mC*mU | AAGGUUCUAAAC | XXXXXXXXXXXXX |
| | | | UAUCCU | |
| WV- | 739 | fC*fU*fC*fU*fU*fU*fC*fC*fA*fU*fG*fG*fA*fA*fG*mG*mU* | CUCUUUCCAUGG | XXXXXXXXXXXXXXXX |
| 34978 | | mU*mC*mU*mA*mA*mA*C*A*A*mU*mC*mC*mU | AAGGUUCUAAAC | XXXXXXXXXXXXX |
| | | | AAUCCU | |
| WV- | 740 | fC*fU*fC*fU*fU*fU*fC*fC*fA*fU*fG*fG*fA*fA*fG*mG*mU* | CUCUUUCCAUGG | XXXXXXXXXXXXXXXX |
| 34979 | | mU*mC*mU*mA*mA*mA*C*L010*A*mU*mC*mC*mU | AAGGUUCUAAAC | XXXXXXXXXXXXX |
| | | | AUCCU | |
| WV- | 741 | fC*SfU*SfC*SfU*SfU*SfU*SfC*SL010*fA*SfU*SfG*SG*SfA* | CUCUUUCAUGGA | SSSSSSSXSSSSSSSSSSSSS |
| 36476 | | SfA*SfG*SmG*SmU*SmU*SmC*SmU*SmA*SmA*SmA*SC* | AGGUUCUAAACC | SSSSSSSS |
| | | SC*SA*SmU*SmC*SmC*SmU | AUCCU | |
| WV- | 742 | fC*SfU*SfC*SfU*SfU*SfU*SfC*SfC*SL010*fU*SfG*SfG*SfA* | CUCUUUCCUGGA | SSSSSSSSXSSSSSSSSSSSS |
| 36477 | | SfA*SfG*SmG*SmU*SmU*SmC*SmU*SmA*SmA*SmA*SC* | AGGUUCUAAACC | SSSSSSSS |
| | | SC*SA*SmU*SmC*SmC*SmU | AUCCU | |
| WV- | 743 | fC*SfU*SfC*SfU*SfU*SfU*SfC*SfC*SfA*SL010*fG*SfG*SfA* | CUCUUUCCAGGA | SSSSSSSSSXSSSSSSSSSSS |
| 36478 | | SfA*SfG*SmG*SmU*SmU*SmC*SmU*SmA*SmA*SmA*SC* | AGGUUCUAAACC | SSSSSSSS |
| | | SC*SA*SmU*SmC*SmC*SmU | AUCCU | |
| WV- | 744 | fC*SfU*SfC*SfU*SfU*SfU*SfC*SfC*SfA*SfU*SL010*fG*SfA* | CUCUUUCCAUGA | SSSSSSSSSSXSSSSSSSSSS |
| 36479 | | SfA*SfG*SmG*SmU*SmU*SmC*SmU*SmA*SmA*SmA*SC* | AGGUUCUAAACC | SSSSSSSS |
| | | SC*SA*SmU*SmC*SmC*SmU | AUCCU | |
| WV- | 745 | fC*SfU*SfC*SfU*SfU*SfU*SfC*SfC*SfA*SfU*SfG*SL010*fA* | CUCUUUCCAUGA | SSSSSSSSSSSXSSSSSSSSS |
| 36480 | | SfA*SfG*SmG*SmU*SmU*SmC*SmU*SmA*SmA*SmA*SC* | AGGUUCUAAACC | SSSSSSSS |
| | | SC*SA*SmU*SmC*SmC*SmU | AUCCU | |
| WV- | 746 | fC*SfU*SfC*SfU*SfU*SfU*SfC*SfC*SfA*SfU*SfG*SfG*SL010* | CUCUUUCCAUGG | SSSSSSSSSSSSXSSSSSSSS |
| 36481 | | fA*SfG*SmG*SmU*SmU*SmC*SmU*SmA*SmA*SmA*SC* | AGGUUCUAAACC | SSSSSSSS |
| | | SC*SA*SmU*SmC*SmC*SmU | AUCCU | |
| WV- | 747 | fC*SfU*SfC*SfU*SfU*SfU*SfC*SfC*SfA*SfU*SfG*SfG*SfA* | CUCUUUCCAUGG | SSSSSSSSSSSSSXSSSSSSS |
| 36482 | | SL010*fG*SmG*SmU*SmU*SmC*SmU*SmA*SmA*SmA*SC* | AGGUUCUAAACC | SSSSSSSS |
| | | SC*SA*SmU*SmC*SmC*SmU | AUCCU | |
| WV- | 748 | fC*SfU*SfC*SfU*SfU*SfU*SfC*SfC*SfA*SfU*SfG*SfG*SfA* | CUCUUUCCAUGG | SSSSSSSSSSSSSSXSSSSSS |
| 36483 | | SfA*SL010*mG*SmU*SmU*SmC*SmU*SmA*SmA*SmA*SC* | AAGUUCUAAACC | SSSSSSSS |
| | | SC*SA*SmU*SmC*SmC*SmU | AUCCU | |
| WV- | 749 | fC*SfU*SfC*SfU*SfU*SfU*SfC*SfC*SfA*SfU*SfG*SfG*SfA* | CUCUUUCCAUGG | SSSSSSSSSSSSSSSXSSSSS |
| 36484 | | SfA*SfG*SL010*mU*SmU*SmC*SmU*SmA*SmA*SmA*SC*SC* | AAGUUCUAAACC | SSSSSSSS |
| | | SA*SmU*SmC*SmC*SmU | AUCCU | |
| WV- | 750 | fC*SfU*SfC*SfU*SfU*SfU*SfC*SfC*SfA*SfU*SfG*SfG*SfA* | CUCUUUCCAUGG | SSSSSSSSSSSSSSSSXSSSS |
| 36485 | | SfA*SfG*SmG*SL010*mU*SmC*SmU*SmA*SmA*SmA*SC* | AAGGUCUAAACC | SSSSSSSS |
| | | SC*SA*SmU*SmC*SmC*SmU | AUCCU | |
| WV- | 751 | fC*SfU*SfC*SfU*SfU*SfU*SfC*SfC*SfA*SfU*SfG*SfG*SfA* | CUCUUUCCAUGG | SSSSSSSSSSSSSSSSSXSSS |
| 36486 | | SfA*SfG*SmG*SmU*SL010*mC*SmU*SmA*SmA*SmA*SC* | AAGGUCUAAACC | SSSSSSSS |
| | | SC*SA*SmU*SmC*SmC*SmU | AUCCU | |
| WV- | 752 | fC*SfU*SfC*SfU*SfU*SfU*SfC*SfC*SfA*SfU*SfG*SfG*SfA* | CUCUUUCCAUGG | SSSSSSSSSSSSSSSSSSXSS |
| 36487 | | SfA*SfG*SmG*SmU*SmU*SL010*mU*SmA*SmA*SmA*SC* | AAGGUUUAAACC | SSSSSSSS |
| | | SC*SA*SmU*SmC*SmC*SmU | AUCCU | |
| WV- | 753 | fC*SfU*SfC*SfU*SfU*SfU*SfC*SfC*SfA*SL010*L010*fG* | CUCUUUCCAGAA | SSSSSSSSSXXSSSSSSSSSS |
| 36488 | | SfA*SfA*SfG*SmG*SmU*SmU*SmC*SmU*SmA*SmA*SmA*SC* | GGUUCUAAACCA | SSSSSSSS |
| | | SC*SA*SmU*SmC*SmC*SmU | UCCU | |
| WV- | 754 | fC*SfU*SfU*SfU*SfU*SfU*SfU*SfC*SfC*SfU*SL010*L010* | CUCUUUCCAUAA | SSSSSSSSSSXXSSSSSSSSS |
| 36489 | | fA*SfA*SfG*SmG*SmU*SmU*SmC*SmU*SmA*SmA*SmA*SC* | GGUUCUAAACCA | SSSSSSSS |
| | | SC*SA*SmU*SmC*SmC*SmU | UCCU | |
| WV- | 755 | fC*SfU*SfC*SfU*SfU*SfU*SfC*SfC*SfA*SL010*L010*L010* | CUCUUUCCAAAG | SSSSSSSSSXXXSSSSSSSS |
| 36490 | | fA*SfA*SfG*SmG*SmU*SmU*SmC*SmU*SmA*SmA*SmA*SC* | GUUCUAAACCAU | SSSSSSSSS |
| | | SC*SA*SmU*SmC*SmC*SmU | CCU | |
| WV- | 756 | fC*SfU*SfC*SfU*SfU*SfU*SfC*SfC*SfA*SfU*SfG*SL010* | CUCUUUCCAUGG | SSSSSSSSSSSXXXSSSSSS |
| 36491 | | L010*L010*fG*SmG*SmU*SmU*SmC*SmU*SmA*SmA*SmA* | GUUCUAAACCAU | SSSSSSSSS |
| | | SC*SC*SA*SmU*SmC*SmC*SmU | CCU | |
| WV- | 757 | fC*SfU*SfC*SfU*SfU*SfU*SfC*SfC*SfA*SfU*SfG*SfG*SfA* | CUCUUUCCAUGG | SSSSSSSSSSSSSSXXSSSSS |
| 36492 | | SfA*SL010*L010*mU*SmU*SmC*SmU*SmA*SmA*SmA*SC* | AAUUCUAAACCA | SSSSSSSS |
| | | SC*SA*SmU*SmC*SmC*SmU | UCCU | |
| WV- | 758 | fC*SfU*SfC*SfU*SfU*SfU*SfC*SfC*SfA*SfU*SfG*SfG*SfA* | CUCUUUCCAUGG | SSSSSSSSSSSSSSSXXSSSS |
| 36493 | | SfA*SfG*SL010*L010*mU*SmC*SmU*SmA*SmA*SmA*SC* | AAGUCUAAACCA | SSSSSSSS |
| | | SC*SA*SmU*SmC*SmC*SmU | UCCU | |
| WV- | 759 | fC*SfU*SfC*SfU*SfU*SfU*SfC*SfC*SfA*SfU*SfG*SfG*SfA* | CUCUUUCCAUGG | SSSSSSSSSSSSSXXXXSSS |
| 36494 | | SfG*L010*L010*L010*mU*SmC*SmU*SmA*SmA*SmA*SC* | AGUCUAAACCAU | SSSSSSSSS |
| | | SC*SA*SmU*SmC*SmC*SmU | CCU | |
| WV- | 760 | fC*SfU*SfC*SfU*SfU*SfU*SfC*SfC*SfA*SL012*fA*SfA*SfG* | CUCUUUCCAAAG | SSSSSSSSSXSSSSSSSSSSS |
| 36495 | | SmG*SmG*SmU*SmC*SmU*SmA*SmA*SmA*SC*SC*SA* | GUUCUAAACCAU | SSSSSS |
| | | SmU*SmC*SmC*SmU | CCU | |
| WV- | 761 | fC*SfU*SfC*SfU*SfU*SfU*SfC*SfC*SfA*SfU*SfG*L012*fG* | CUCUUUCCAUGG | SSSSSSSSSSXXSSSSSSSSS |
| 36496 | | SmG*SmU*SmU*SmC*SmU*SmA*SmA*SmA*SC*SC*SA*SmU* | GUUCUAAACCAU | SSSSSS |
| | | SmC*SmC*SmU | CCU | |
| WV- | 762 | fC*SfU*SfC*SfU*SfU*SfU*SfC*SfC*SfA*SfU*SfG*SfG*SfA* | CUCUUUCCAUGG | SSSSSSSSSSSSSXSSSSSSS |
| 36497 | | SL012*mU*SmU*SmC*SmU*SmA*SmA*SmA*SC*SC*SA*SmU* | AUUCUAAACCAU | SSSSSS |
| | | SmC*SmC*SmU | CCU | |
| WV- | 763 | fC*SfU*SfC*SfU*SfU*SfU*SfC*SfC*SfA*SL012*fA*SfA* | CUCUUUCCAAAG | SSSSSSSSSXSSXSSSSSSSS |
| 36498 | | SL012*mU*SmC*SmU*SmA*SmA*SmA*SC*SC*SA*SmU*SmC* | CUAAACCAUCCU | SSSS |
| | | SmC*SmU | | |
| WV- | 764 | fC*SfU*SfC*SfU*SfU*SfU*SfC*SfC*SfA*SL028*U*SfA*SfG* | CUCUUUCCAAAG | SSSSSSSSSXSSSSSSSSSSS |
| 36499 | | SmG*SmU*SmU*SmC*SmU*SmA*SmA*SmA*SC*SC*SA* | GUUCUAAACCAU | SSSSSS |
| | | SmU*SmC*SmC*SmU | CCU | |
| WV- | 765 | fC*SfU*SfC*SfU*SfU*SfU*SfC*SfC*SfA*SfU*SfG*L028*fG* | CUCUUUCCAUGG | SSSSSSSSSSXXSSSSSSSSS |
| 36500 | | SmG*SmU*SmU*SmC*SmU*SmA*SmA*SmA*SC*SC*SA*SmU* | GUUCUAAACCAU | SSSSSS |
| | | SmC*SmC*SmU | CCU | |
| WV- | 766 | fC*SfU*SfC*SfU*SfU*SfU*SfC*SfC*SfA*SfU*SfG*SfG*SfA* | CUCUUUCCAUGG | SSSSSSSSSSSSSXSSSSSSS |
| 36501 | | SL028*mU*SmU*SmC*SmU*SmA*SmA*SmA*SC*SC*SA*SmU* | AUUCUAAACCAU | SSSSSS |
| | | SmC*SmC*SmU | CCU | |
| WV- | 767 | fC*SfU*SfC*SfU*SfU*SfU*SfC*SfC*SfA*SL028*fA*SfA* | CUCUUUCCAAAG | SSSSSSSSSXSSXSSSSSSSS |
| 36502 | | SL028*mU*SmC*SmU*SmA*SmA*SmA*SC*SC*SA*SmU*SmC* | CUAAACCAUCCU | SSSS |
| | | SmC*SmU | | |

TABLE 1H
Example oligonucleotides and/or compositions that target SERPINAl1

| | SEQ | | | Stereochemistry/ |
|---|---|---|---|---|
| ID | ID NO | Description | Base Sequence | Linkage |
| WV- | 768 | mC*mC*mC*mC*rA*rG*rC*rA*rG*rC*rA*rU*rC*rA*rC*rU*rC*rC*rC* | CCCCAGCAGCAU | XXXXXXXXXXXXX |
| 27822 | | rU*rU*rU*rC*rU*rC*I*rU*mC*mG*mA | CACUCCCUUUCU | XXXXXXXXXXXXX |
| | | | CIUCGA | XXX |
| WV- | 769 | fC*SfC*SfC*SC*SA*SG*SC*SA*SG*SC*SfA*SfU*SC*SfA*SfC* | CCCCAGCAGCAU | SSSSSSSSSSSSSSSSS |
| 30296 | | SmU*SmC*SmC*SmC*SmU*SmU*SmU*SmC*ST*SC*SI*mU*SmC* | CACUCCCUUUCT | SSSSSSSSXSSS |
| | | SmG*SmA | CIUCGA | |
| WV- | 770 | fC*SfC*SfC*SfC*SfA*SG*SfC*SfA*SfG*SfC*SfU*SfU*SfC*SfA*SfG* | CCCCAGCAGCUU | SSSSSSSSSSSSSSSSS |
| 30297 | | SmU*SmC*SmC*SmC*SmU*SmU*SmU*SmC*ST*SC*SI*mU*SmC* | CAGUCCCUUUCT | SSSSSSSSXSSS |
| | | SmG*SmA | CIUCGA | |
| WV- | 771 | fC*SfC*SfC*SfC*SfA*SfG*SfC*SfA*SfG*SfC*SfA*SfU*SfC*SfA* | CCCCAGCAGCAU | SSSSSSSSSSSSSSSSS |
| 31764 | | SfC*SmU*SmC*SmC*SmC*SmU*SmU*SmU*SmC*ST*SC*Sb0031*mU* | CACUCCCUUUCT | SSSSSSSSXSSS |
| | | SmC*SmG*SmA | CIUCGA | |
| WV- | 772 | fC*SfC*SfC*SfC*SfA*SfG*SfC*SfA*SfG*SfC*SfU*SfU*SfC*SfA* | CCCCAGCAGCUU | SSSSSSSSSSSSSSSSS |
| 31765 | | SfG*SmU*SmC*SmC*SmC*SmU*SmU*SmU*SmC*ST*SC*Sb0031*mU* | CAGUCCCUUUCT | SSSSSSSSXSSS |
| | | SmC*SmG*SmA | CIUCGA | |
| WV- | 773 | fC*SfC*SfC*SfC*SfA*SfG*SfC*SfA*SfG*SfC*SfA*SfU*SfC*SfA* | CCCCAGCAGCAU | SSSSSSSSSSSSSSSSS |
| 31768 | | SfC*SmU*SmC*SmC*SmC*SmU*SmU*SmU*SmC*ST*SC*Sb001G* | CACUCCCUUUCT | SSSSSSSSXSSS |
| | | mU*SmC*SmG*SmA | CGUCGA | |
| WV- | 774 | fC*SfC*SfC*SfC*SfA*SfG*SfC*SfA*SfG*SfC*SfU*SfU*SfC*SfA* | CCCCAGCAGCUU | SSSSSSSSSSSSSSSSS |
| 31769 | | SfG*SmU*SmC*SmC*SmC*SmU*SmU*SmU*SmC*ST*SC*Sb001G* | CAGUCCCUUUCT | SSSSSSSSXSSS |
| | | mU*SmC*SmG*SmA | CGUCGA | |
| WV- | 775 | fC*SfC*SfC*SfC*SfA*SfG*SfC*SfA*SfG*SfC*SfA*SfU*SfC*SfA* | CCCCAGCAGCAU | SSSSSSSSSSSSSSSSS |
| 37915 | | SfC*SmU*SmC*SmC*SmC*SmU*SmU*SmU*SmC*ST*Sb001A*SI*mU* | CACUCCCUUUCT | SSSSSSSSXSSS |
| | | *SmC*SmG*SmA | AIUCGA | |
| WV- | 776 | fC*SfC*SfC*SfC*SfA*SfG*SfC*SfA*SfG*SfC*SfU*SfU*SfC*SfA* | CCCCAGCAGCUU | SSSSSSSSSSSSSSSSS |
| 37916 | | SfG*SmU*SmC*SmC*SmC*SmU*SmU*SmU*SmC*ST*Sb001A*SI*mU* | CAGUCCCUUUCT | SSSSSSSSXSSS |
| | | SmC*SmG*SmA | AIUCGA | |
| WV- | 777 | fC*SfC*SfC*SfC*SfA*SfG*SfC*SfA*SfG*SfC*SfA*SfU*SfC*SfA* | CCCCAGCAGCAU | SSSSSSSSSSSSSSSSS |
| 37917 | | SfCSmU*SmC*SmC*SmC*SmU*SmU*SmU*SmC*ST*Sb002A*SI*mU* | CACUCCCUUUCT | SSSSSSSSXSSS |
| | | SmC*SmG*SmA | AIUCGA | |
| WV- | 778 | fC*SfC*SfC*SfC*SfA*SAG*SfC*SfA*SfG*SFA*SfU*SfU*SfC*SfA* | CCCCAGCAGCUU | SSSSSSSSSSSSSSSSS |
| 37918 | | SfG*SmU*SmC*SmC*SmC*SmU*SmU*SmU*SmC*ST*Sb002A*SI* | CAGUCCCUUUCT | SSSSSSSSXSSS |
| | | mU*SmC*SmG*SmA | AIUCGA | |
| WV- | 779 | fC*SfC*SfC*SfC*SfA*SfG*SfC*SfA*SfG*SfC*SfA*SfU*SfC*SfA* | CCCCAGCAGCAU | SSSSSSSSSSSSSSSSS |
| 37919 | | SfC*SmU*SmC*SmC*SmC*SmU*SmU*SmU*SmC*ST*Sb003A*SI*mU* | CACUCCCUUUCT | SSSSSSSSXSSS |
| | | SmC*SmG*SmA | AIUCGA | |
| WV- | 780 | fC*SfC*SfC*SfC*SfA*SfG*SfC*SfA*SfG*SfC*SfU*SfU*SfC*SfA* | CCCCAGCAGCUU | SSSSSSSSSSSSSSSSS |
| 37920 | | SfG*SmU*SmC*SmC*SmC*SmU*SmU*SmU*SmC*ST*Sb003A*SI* | CAGUCCCUUUCT | SSSSSSSSXSSS |
| | | mU*SmC*SmG*SmA | AIUCGA | |
| WV- | 781 | fC*SfC*SfC*SfC*SfA*SfG*SfC*SfA*SfG*SfC*SfA*SfU*SfC*SfA* | CCCCAGCAGCAU | SSSSSSSSSSSSSSSSS |
| 37921 | | SfC*SmU*SmC*SmC*SmC*SmU*SmU*SmU*SmC*ST*Sb009U*SI*mU* | CAGUCCCUUUCT | SSSSSSSSXSSS |
| | | SmC*SmG*SmA | UIUCGA | |
| WV- | 782 | fC*SfC*SfC*SfC*SfA*SfG*SfC*SfA*SfG*SfC*SfU*SfU*SfC*SfA* | CCCCAGCAGCUU | SSSSSSSSSSSSSSSSS |
| 37922 | | SfG*SmU*SmC*SmC*SmC*SmU*SmU*SmU*SmC*ST*Sb009U*SI* | CAGUCCCUUUCT | SSSSSSSSXSSS |
| | | mU*SmC*SmG*SmA | UIUCGA | |
| WV- | 783 | fC*SfC*SfC*SfC*SfA*SfG*SfC*SfA*SfG*SfC*SfA*SfU*SfC*SfA* | CCCCAGCAGCAU | SSSSSSSSSSSSSSSSS |
| 37923 | | SfC*SmU*SmC*SmC*SmC*SmU*SmU*SmU*SmC*ST*Sb001A*SI* | CAGUCCCUUUCT | SSSSSSSSSSSS |
| | | SmU*SmC*SmG*SmA | AIUCGA | |
| WV- | 784 | fC*SfC*SfC*SfC*SfA*SfG*SfC*SfA*SfG*SfC*SfU*SfU*SfC*SfA* | CCCCAGCAGCUU | SSSSSSSSSSSSSSSSS |
| 37924 | | SfG*SmU*SmC*SmC*SmC*SmU*SmU*SmU*SmC*ST*Sb001A*SI*SmU* | CAGUCCCUUUCT | SSSSSSSSSSSS |
| | | SmC*SmG*SmA | AIUCGA | |
| WV- | 785 | fC*SfC*SfC*SfC*SfA*SfG*SfC*SfA*SfG*SfC*SA*SfU*SfC*SA*SfC* | CCCCAGCAGCAU | SSSSSSSSSSSSSSSSS |
| 37925 | | SmU*SmC*SmC*SmC*SmU*SmU*SmU*SmC*ST*Sb001A*SIn001RMU* | CAGUCCCUUUCT | SSSSSSSSnRSSS |
| | | SmC*SmG*SmA | AIUCGA | |
| WV- | 786 | fC*SfC*SC*SfC*SfA*SfG*SfC*SfA*SfG*SfC*SfU*SfU*SfC*SfA*SfG* | CCCCAGCAGCUU | SSSSSSSSSSSSSSSSS |
| 37926 | | SmU*SmC*SmC*SmC*SmU*SmU*SmU*SmC*ST*Sb001A*SIn001RmU* | CAGUCCCUUUCT | SSSSSSSSnRSSS |
| | | SmC*SmG*SmA | AIUCGA | |
| WV- | 787 | fCn001RfC*SfC*SfC*SfA*SfG*SfC*SfA*SfG*SfC*SfA*SfU*SfC* | CCCCAGCAGCAU | nRSSSSSSSSSSSSnR |
| 38619 | | SfAn001RfC*SmUn001RmC*SmC*SmC*SmU*SmU*SmU*SmC*ST*SC* | CAGUCCCUUUCT | SnRSSSSSSSSSnXSS |
| | | SIn001mU*SmC*SmGn001RmA | CIUCGA | nR |
| WV- | 788 | fCn001RfC*SfC*SfC*SfA*SfG*SfC*SfA*SfG*SfC*SfU*SfU*SfC* | CCCCAGCAGCUU | nRSSSSSSSSSSSSnR |
| 38620 | | SfAn001RfG*SmUn001RmC*SmC*SmC*SmU*SmU*SmU*SmC*ST*SC* | CAGUCCCUUUCT | SnRSSSSSSSSSnXSS |
| | | SIn001mU*SmC*SmGn001RmA | CIUCGA | nR |
| WV- | 789 | fCn001RfC*SfC*SfC*SfA*SfG*SfC*SfA*SfG*SfC*SfU*SfU*SfC* | CCCCAGCAGCUU | nRSSSSSSSSSSSSnR |
| 38621 | | SfAn001RfG*SinUn001RmC*SmC*SmC*SmU*SmU*SmU*SmC*ST*SrC* | CAGUCCCUUUCT | SnRSSSSSSSSSnXSS |
| | | SIn001mU*SmC*SmGn001RmA | CIUCGA | nR |
| WV- | 790 | fCn001RfC*SfC*SfC*SfA*SfG*SfC*SfA*SfG*SfC*SfU*SfU*SfC* | CCCCAGCAGCUU | nRSSSSSSSSSSSSnR |
| 38622 | | SfAn001RfG*SmUn001RmC*SmC*SmC*SmU*SmU*SmU*SmC*ST* | CAGUCCCUUUCT | SnRSSSSSSSSSnXSS |
| | | Sb001A*SIn001mU*SmC*SmGnGn001RmA | AIUCGA | nR |
| WV- | 791 | L025L025L025fCn001RfC*SfC*SfC*SfA*SG*SfC*SfA*SfG*SfC*SfA* | CCCCAGCAGCAU | OOOnRSSSSSSSSSSS |
| 38623 | | SfU*SfC*SfAn001RfC*SmUn001RmC*SmC*SmC*SmU*SmU*SmU* | CACUCCCUUUCT | SnRSnRSSSSSSSSSn |
| | | SmC*ST*SC*SIn001mU*SmC*SmGn001RmA | CIUCGA | XSSnR |
| WV- | 792 | L025L025L025fCn001RfC*SfC*SfC*SfA*SfG*SfC*SfA*SfG*SfC*SfU* | CCCCAGCAGCUU | OOOnRSSSSSSSSSSS |
| 38624 | | SfU*SfC*SfAn001RfG*SmUn001RmC*SmC*SmC*SmU*SmU*SmU* | CAGUCCCUUUCT | SnRSnRSSSSSSSSSn |
| | | SmC*ST*SC*SIn001mU*SmC*SmGn00iRmA | CIUCGA | XSSnR |
| WV- | 793 | L025L025L025fCn001RfC*SfC*SfC*SfA*SfG*SfC*SfA*SfG*SfC*SfU* | CCCCAGCAGCUU | OOOnRSSSSSSSSSSS |
| 38625 | | SfU*SfC*SfAn001RfG*SmUn001RmC*SmC*SmC*SmU*SmU*SmU* | CAGUCCCUUUCT | SnRSnRSSSSSSSSSn |
| | | SmC*ST*SrC*SIn001mU*SmC*SmGn001RmA | CIUCGA | XSSnR |
| WV- | 794 | L025L0251025fCn001RfC*SfC*SfC*SfA*SfG*SfC*SfA*SfG*SfC*SfU* | CCCCAGCAGCUU | OOOnRSSSSSSSSSSS |
| 38626 | | SfU*SfC*SfAn001RfG*SmUn001RmC*SmC*SmC*SmU*SmU*SmU* | CAGUCCCUUUCT | SnRSnRSSSSSSSSSn |
| | | SmC*ST*Sb001A*SIn001mU*SmC*SmGn001RmA | ATUCGA | XSSnR |
| WV- | 795 | fCn001RfC*SfC*SfC*StA*SfG*SfC*SfA*SfG*SfC*SfU*SfU*SfC*SfA* | CCCCAGCAGCUU | nRSSSSSSSSSSSSSS |
| 38628 | | SfG*SmU*SmC*SmC*SmC*SmU*SmU*SmU*SmC*ST*SC*SIn001mU* | CAGUCCCUUUCT | SSSSSSSSSSnXSSnR |
| | | SmC*SmGn001RmA | CIUCGA | |
| WV- | 796 | fCn001RfC*SfC*SfC*SfA*SfG*SfC*SfA*SfG*SfC*SfU*SfU*SfC*SfA* | CCCCAGCAGCUU | nRSSSSSSSSSSSSSS |
| 38629 | | SfG*SmU*SmC*SmC*SmC*SmU*SmU*SmU*SmC*ST*SrC*SIn001mU* | CAGUCCCUUUCT | SSSSSSSSSSnXSSnR |
| | | SmC*SmGn001RmA | CIUCGA | |
| WV- | 797 | fCn001RfC*SfC*SfC*SfA*SfG*SfC*SfA*SfG*SfC*SfU*SfU*SfC*SfA* | CCCCAGCAGCUU | nRSSSSSSSSSSSSSS |
| 38630 | | SfG*SmU*SmC*SmC*SmC*SmU*SmU*SmU*SmC*ST*Sb001A*Sin001mU* | CAGUCCCUUUCT | SSSSSSSSSSnXSSnR |
| | | SmC*SmGn001RmA | ATUCGA | |
| WV- | 798 | L025L0251025fCn001RfC*SfC*SfC*SfA*SfG*SfC*SfA*SfG*SfC*SfU* | CCCCAGCAGCUU | OOOnRSSSSSSSSSSS |
| 38631 | | SfU*SfC*SfA*SfG*SmU*SmC*SmC*SmC*SmU*SmU*SmU*SmC* | CAGUCCCUUUCT | SSSSSSSSSSSSSnXS |
| | | ST*SC*SIn001mU*SmC*SmGn001RmA | CIUCGA | SnR |
| WV- | 799 | L02510251025fCn001RfC*SfC*SfC*SfA*SfG*SfC*SfA*SfG*SfC*SfU* | CCCCAGCAGCUU | OOOnRSSSSSSSSSSS |
| 38632 | | SfU*SfC*SfA*SfG*SmU*SmC*SmC*SmC*SmU*SmU*SmU*SmC* | CAGUCCCUUUCT | SSSSSSSSSSSSSnXS |
| | | ST*SrC*SIn001mU*SmC*SmGn001RmA | CIUCGA | SnR |
| WV- | 800 | fCn001RfC*SfC*SfC*SfA*SfG*SfC*SfA*SfG*SfC*SfU*SfU*SfC* | CCCCAGCAGCUU | nRSSSSSSSSSSSSnR |
| 38923 | | SfAn0001RfG*SmUn001RmC*SmC*SmC*SmU*SmU*SmU*SmC*ST* | CAGUCCCUUUCT | SnRSSSSSSSSSnXSS |
| | | Sb008U*Gn001mU*SmC*SmGn001RmA | UGUCGA | nR |
| WV- | 801 | L025L025L025fUn001RfC*SfA*SfG*SfC*SfA*SfG*SfA*SfG*SfC*SfC* | UCAGCACAGCCU | OOOnRSSSSSSSSSSS |
| 39097 | | SfU*SfU*SfA*SfU*SmG*SmC*SmA*SmC*SmG*SmG*SmC*SmC* | UAUGCACGGCCU | SSSSSSSSSSSSSnXS |
| | | SfU*SC*SIn001mG*SmA*SmGn001RmA | CIGAGA | SnR |
| WV- | 802 | L02510251025fUn001RfC*SfG*SfU*SfC*SAPSOUSfU*SfG*SfG*SfU* | UCGUCGAUGGUC | OOOnRSSSSSSSSSSS |
| 39104 | | SfU*SfA*SfG*SfC*SmA*SmC*SmA*SmG*SmC*SmC*SmU*SmU* | AGCACAGCCUUA | SSSSSSSSSSSSSnXS |
| | | SfA*SC*SIn001mC*SmA*SmCn001RmG | CICACG | SnR |
| WV- | 803 | L02510251025fCn001RfA*SfG*SfC*SfU*SfU*SfC*SfA*SfG*SfU*SfU* | CAGCUUCAGUCC | OOOnRSSSSSSSSSSS |
| 39105 | | SfC*SfC*SfU*SfU*SmU*SmC*SmU*SmC*SmG*SmU*SmC*SmG* | CUUUCUCGUCGA | SSSSSSSSSSSSSnXS |
| | | SfA*SC*SIn001mG*SmU*SmCn001RmA | CIGUCA | SnR |
| WV- | 804 | Mod001L001fUn001Rfc*SfG*SfU*SfC*SfG*SfA*SfU*SfG*SfG*SfU* | UCGUCGAUGGUC | OnRSSSSSSSSSSSSS |
| 39567 | | SfC*SfA*SfG*SfC*SmA*SmC*SmA*SmG*SmC*SmC*SmU*SmU*Sfa* | AGCACAGCCUUA | SSSSSSSSSSSnSSSnR |
| | | SC*SIn001SmC*SmA*SmCn001RmG | CICACG | |
| WV- | 805 | Mod001L001fCn001RfU*SfC*SFg*SfU*SfC*SfG*SFa*SfU*SfG*SfG* | CUCGUCGAUGGU | OnRSSSSSSSSSSSSS |
| 39568 | | SfU*SfC*SfA*SfG*SmC*SmA*SmC*SmA*SmG*SmC*SmC*SmU* | CAGCACAGCCUU | SSSSSSSSSSSSnSSnR |
| | | Smu*SfA*SC*SIn001SmC*SmAn001RmC | ACICAC | |
| WV- | 806 | Mod001L001fGn001RfU*SfC*SfG*SfA*SfU*SfG*SfG*SfU*SfC*SfA* | GUCGAUGGUCAG | OnRSSSSSSSSSSSSS |
| 39570 | | SfG*SfC*SfA*SfC*SmA*SmG*SmC*SmC*SmU*SmU*SfA*SC*SIn01SmC* | CACAGCCUUACIC | SSSSSSSSSnSSSSSnR |
| | | SmA*SmC*SmG*SmGn001RmC | ACGGC | |
| WV- | 807 | Mod001L001fUn001RC*SfG*SfA*SfU*SfG*SfG*SfU*SfC*SU*SfG* | UCGAUGGUCAGC | OnRSSSSSSSSSSSSS |
| 39571 | | SfC*SfA*SfC*SfA*SmG*SmC*SmC*SmU*SmU*SfA*SC*SIn001SmC* | ACAGCCUUACICA | SSSSSSSSnSSSSSSnR |
| | | SmA*SmC*SmG*SmG*SmCn001RmC | CGGCC | |
| WV- | 808 | Mod001L001fUn001RfC*SfG*SfU*SfUSfG*SfA*SfU*SfG*SfG*SfU* | UCGUCGAUGGUC | OnRSSSSSSSSSSSSS |
| 39572 | | SfC*SfA*SfG*SfC*SmA*SmC*SmA*SmG*SmC*SmC*SmU*SmU*SfA* | AGCACAGCCUUA | SSSSSSSSSSSnSSSnR |
| | | SC*SIn001SmC*SfA*SmCn001RmG | CICACG | |
| WV- | 809 | Mod001L001fUn001RfC*SfG*SfU*SfUSfG*SfA*SfU*SfG*SfG*SfU* | UCGUCGAUGGUC | OnRSSSSSSSSSSSSS |
| 39573 | | SfC*SfA*SfG*SfC*SmA*SmC*SmA*RmG*SmC*SmC*SmU*SmU*SfA* | AGCACAGCCUUA | SSSRSSSSSSSnSSSn |
| | | SC*SIn001SmC*SmA*SmCn001RmG | CICACG | R |
| WV- | 810 | Mod001L001fUn001RfC*SfG*SfU*SfC*SfG*SfA*SfU*SfG*SfG*SfU* | UCGUCGAUGGUC | OnRSSSSSSSSSSSSS |
| 39574 | | SfC*SfA*SfG*SfC*SmA*SmC*SmA*RmG*SmC*SmC*SmU*SmU*SfA* | AGCACAGCCUUA | SSSRSSSSSSRnSSSn |
| | | SC*RIn001SmC*SfA*SmCn001RmG | CICACG | R |
| WV- | 811 | Mod001L001fUn001RfC*SfU*SfC*SFG*SfU*SfC*SFG*SfA*SfU*SfG* | UCUCGUCGAUGG | OnRSSSSSSSSSSSSS |
| 39575 | | SfG*SfU*SfC*SfA*SmG*SmC*SmA*SmC*SmA*SmG*SmC*SmC*SfU* | UCAGCACAGCCU | SSSSSSSSSSSnRSSn |
| | | SC*SAn001RmU*SmG*SmCn001RmA | CAUGCA | R |
| WV- | 812 | Mod001L001fUn001RfU*SfC*SfU*SfC*SfG*SfU*SfC*SfG*SfA*SfU* | UUCUCGUCGAUG | OnRSSSSSSSSSSSSS |
| 39576 | | SfG*SfG*SfU*SfC*SmA*SmG*SmC*SmA*SmC*SmA*SmG*SmC* | GUCAGCACAGCC | SSSSSSSSSSSSnRSn |
| | | SmC*SfU*SC*SAn001RmU*SmGn001RmC | UCAUGC | R |
| WV- | 813 | Mod001L001fCn001RfU*SfC*SfG*SfU*SfC*SfG*SfA*SfU*SfG*SfG* | CUCGUCGAUGGU | OnRSSSSSSSSSSSSS |
| 39577 | | SfU*SfC*SfA*SfG*SmC*SmA*SmC*SmA*SmG*SmC*SmC*SfU*SC* | CAGCACAGCCUC | SSSSSSSSSSnRSSSn |
| | | SAn001RmU*SmG*SmC*SmAn001RmC | AUGCAC | R |
| WV- | 814 | Mod001L001fUn001RfC*SfG*SfU*SfC*SfG*SfA*SfU*SfG*SfG*SfU* | UCGUCGAUGGUC | OnRSSSSSSSSSSSSS |
| 39578 | | SfC*SfA*SfG*SfC*SmA*SmC*SmA*SmG*SmC*SmC*SfU*SC* | AGCACAGCCUCA | SSSSSSSSSnRSSSSn |
| | | SAn001RmU*SmG*SmC*SmA*SmCn001RmG | UGCACG | R |
| WV- | 815 | Mod001L001fCn001RfG*SfU*SfC*SfG*SfA*SfU*SfG*SfG*SfU*SfC* | CGUCGAUGGUCA | OnRSSSSSSSSSSSSS |
| 39579 | | SfA*SfG*SfC*SfA*SmC*SmA*SmG*SmC*SmC*SfU*SC*SAn001RmU* | GCACAGCCUCAU | SSSSSSSSnRSSSSSn |
| | | SmG*SmC*SmA*SmC*SmGn001RmG | GCACGG | R |
| WV- | 816 | Mod001L001fUn001RfC*SfU*SfC*SfG*SfU*SfC*SfG*SfA*SfU*SfG* | UCUCGUCGAUGG | OnRSSSSSSSSSSSSS |
| 39580 | | SfG*SfU*SfC*SfA*SmG*SmC*SmA*SmC*SmA*SmG*SmC*SmC*SfU* | UCAGCACAGCCU | SSSSSSSSSSSnRSSn |
| | | SC*SAn001RmU*SfG*SmCn001RmA | CAUGCA | R |
| WV- | 817 | Mod001L001fUn001RfC*SfU*SfC*SfG*SfU*SfC*SfG*SfA*SfU*SfG* | UCUCGUCGAUGG | OnRSSSSSSSSSSSSS |
| 39581 | | SfG*SfU*SfC*SfA*SmG*SmC*SmA*RmC+SmA*SmG*SmC*SmC*SfU* | UCAGCACAGCCU | SSSRSSSSSSSnRSSn |
| | | SC*SAn001RmU*SmG*SmCn001RmA | CAUGCA | R |
| WV- | 818 | Mod001L001fUn001RfC*SfU*SfU*SfG*SfU*SfC*SfG*SfA*SfU*SfG* | UCUCGUCGAUGG | OnRSSSSSSSSSSSSS |
| 39582 | | SfG*SfU*SfC*SfA*SmG*SmC*SmA*RmC+SmA*SmG*SmC*SmC*SfU* | UCAGCACAGCCU | SSSRSSSSSSRnRSSn |
| | | SC*RAn001RmU*SfG*SmCn001RmA | CAUGCA | R |
| WV- | 819 | Mod001L001fUn001RfC*SfA*SfG*SfC*SfA*SfC*SfA*SfG*SfC*SfC* | UCAGCACAGCCU | OnRSSSSSSSSSSSSS |
| 39583 | | SfU*SfU*SfA*SfU*SmG*SmC*SmA*SmC*SmG*SmG*SmC*SmC*SfU* | UAUGCACGGCCU | SSSSSSSSSSSnSSSnR |
| | | SC*SIn001SmG*SmA*SmGn001RmA | CIGAGA | |
| WV- | 820 | Mod001L001Rm001RfU*SfC*SfA*SfG*SfC*SfA*SfC*SfA*SfG*SfC* | GUCAGCACAGCC | OnRSSSSSSSSSSSSS |
| 39584 | | SfC*SfU*SfU*SfA*SmU*SmG*SmC*SmA*SmC*SmG*SmG*SmC* | UUAUGCACGGCC | SSSSSSSSSSSSnSSnR |
| | | SmC*SfU*SC*SIn001SmG*SmAn001RmG | UCIAG | |
| WV- | 821 | Mod001L001Rm001RfA*SfG*SfC*SfA*SfC*SfA*SfG*SfC*SfC*SfU* | CAGCACAGCCUU | OnRSSSSSSSSSSSSS |
| 39585 | | SfU*SfA*SfU*SfG*SmC*SmA*SmC*SmG*SmG*SmC*SmC*SfU*SC* | AUGCACGGCCUCI | SSSSSSSSSSnSSSSnR |
| | | SIn001SmG*SmA*SmG*SmAn001RmG | GAGAG | |
| WV- | 822 | Mod001L001fAn001RfG*SfC*SfA*SfC*SfA*SfG*SfC*SfC*SfU*SRU | AGCACAGCCUUA | OnRSSSSSSSSSSSSS |
| 39586 | | SfA*SfU*SfG*SfC*SmA*SmC*SmG*SmG*SmC*SmC*SfU*SC* | UGCACGGCCUCIG | SSSSSSSSSnSSSSSnR |
| | | SIn01SmG*SmA*SmG*SmA*SmGn001RmC | AGAGC | |
| Wy- | 823 | Mod001L001fGn001RfC*SfA*SfC*SfA*SfG*SfC*SfC*SfU*SfU*SfA* | GCACAGCCUUAU | OnRSSSSSSSSSSSSS |
| 39587 | | SfC*SfG*SfC*SfA*SmC*SmG*SmG*SmC*SmC*SfU*SC*SIn001SmG* | GCACGGCCUCIGA | SSSSSSSSuSSSSSSnR |
| | | SmA*SmG*SmA*SmG*SmCn001RmU | GAGCU | |
| WV- | 824 | Mod001L001Rm001RfC*SfA*SfG*SfC*SfA*SfC*SfA*SfG*SfC*SfC* | UCAGCACAGCCU | OnRSSSSSSSSSSSSS |
| 39588 | | SfU*SfU*SfA*SfU*SmG*SmC*SmA*SmC*SmG*SmG*SmC*SmC*SfU* | UAUGCACGGCCU | SSSSSSSSSSSnSSSnR |
| | | SC*SIn001SmG*SfA*SmGn001RmA | CIGAGA | |
| WV- | 825 | Mod001L001fUn001RfC*SfA*SfG*SfC*SfA*SfC*SfA*SfG*SfC*SfC* | UCAGCACAGCCU | OnRSSSSSSSSSSSSS |
| 39589 | | SfU*SfU*SfA*SfU*SmG*SmC*SmA*RmC*SmG*SmG*SmC*SmC*SfU* | UAUGCACGGCCU | SSSRSSSSSSSnSSSn |
| | | SC*SIn001SmG*SmA*SmGn001RmA | CIGAGA | R |
| WV- | 826 | Mod001L001fUn001RfC*SfA*SfG*SfC*SfA*SfC*SfA*SfG*SfC*SfC* | UCAGCACAGCCU | OnRSSSSSSSSSSSSS |
| 39590 | | SfU*SfU*SfA*SfU*SmG*SmC*SmA*RmC*SmG*SmG*SmC*SmC*SfU* | UAUGCACGGCCU | SSSRSSSSSSRnSSSn |
| | | SC*RIn001SmG*SfA*SmGn001RmA | CIGAGA | R |
| WV- | 827 | Mod001L001fCn001RfA*SfG*SfC*SfU*SfU*SfC*SOUSfG*SfU*SC* | CAGCUUCAGUCC | OnRSSSSSSSSSSSSS |
| 39591 | | SfC*SfU*SfU*SfU*SmU*SmC*SmU*SmC*SmG*SmU*SmC*SmG*SfA* | CUUUCUCGUCGA | SSSSSSSSSSSnSSSnR |
| | | SC*SIn001SmG*SmU*SmCn001RmA | CIGUCA | |
| WV- | 828 | Mod001L001fGn001RfC*SfA*SfG*SfC*SfU*SfU*SfC*SfA*SfG*SfU* | GCAGCUUCAGUC | OnRSSSSSSSSSSSSS |
| 39592 | | SfC*SfC*SfC*SfU*SmU*SmU*SmC*SmU*SmC*SmG*SmU*SmC* | CCUUUCUCGUCG | SSSSSSSSSSSSnSSnR |
| | | SmG*SfA*SC*SIn001SmG*SmUn001RmC | ACIGUC | |
| WV- | 829 | Mod001L001fAn001RG*SfC*SfU*SfU*SfC*SfA*SfG*SfU*SfC*SfC* | AGCUUCAGUCCC | OnRSSSSSSSSSSSSS |
| 39593 | | SfC*SfU*SfU*SfU*SmC*SmU*SmC*SmG*SmU*SmC*SmG*SfA*SC* | UUUCUCGUCGACI | SSSSSSSSSSnSSSSnR |
| | | SIn001SmG*SmU*SmC*SmAn001RmG | GUCAG | |
| WV- | 830 | Mod001L001fGn001RfC*SfU*SfU*SfC*SfA*SfG*SfU*SfC*SfC*SfC* | GCUUCAGUCCCU | OnRSSSSSSSSSSSSS |
| 39594 | | SfU*SfU*SfU*SfC*SmU*SmC*SmG*SmU*SmC*SmG*SfA*SC* | UUCUCGUCGACIG | SSSSSSSSSnSSSSSnR |
| | | SIn001SmG*SmU*SmC*SmA*SmGn001RmC | UCAGC | |
| WV- | 831 | Mod001L001fCn001RfU*SfU*SfC*SfA*SfG*SfU*SC*SfC*SfC*SfU* | CUUCAGUCCCUU | OnRSSSSSSSSSSSSS |
| 39595 | | SfU*SfU*SfC*SfU*SmC*SmG*SmU*SmC*SmG*SfA*SC*SIn001SmG* | UCUCGUCGACIGU | SSSSSSSSnSSSSSSnR |
| | | SmU*SmC*SmA*SmG*SmCn001RmA | CAGCA | |
| WV- | 832 | Mod001L001fCn001RfA*SfG*SfC*SfU*SfU*SfC*SfA*SfG*SfU*SfC* | CAGCUUCAGUCC | OnRSSSSSSSSSSSSS |
| 39596 | | SfC*SfC*SfU*SfU*SmU*SmC*SmU*SmC*SmG*SmU*SmC*SmG*SfA* | CUUUCUCGUCGA | SSSSSSSSSSSnSSSnR |
| | | SC*SIn001SmG*SfU*SmCn001RmA | CIGUCA | |
| WV- | 833 | Mod001L001fCn001RfA*SfG*SfC*SfU*SfU*SfC*SfA*SfG*SfU*SfC* | CAGCUUCAGUCC | OnRSSSSSSSSSSSSS |
| 39597 | | SfC*SfC*SfU*SfU*SmU*SmC*SmU*RmC*SmG*SmU*SmC*SmG*SfA* | CUUUCUCGUCGA | SSSRSSSSSSSnSSSn |
| | | SC*SIn001SmG*SmU*SmCn001RmA | CIGUCA | R |
| WV- | 834 | Mod001L001fCn001RfA*SfG*SfC*SfU*SfU*SfC*SfA*SfG*SfU*SfC* | CAGCUUCAGUCC | OnRSSSSSSSSSSSSS |
| 39598 | | SfC*SfC*SfU*SfU*SmU*SmC*SmU*RmC*SmG*SmU*SmC*SmG*SfA* | CUUUCUCGUCGA | SSSRSSSSSSRnSSSn |
| | | SC*RIn001SmG*SfU*SmCn001RmA | CIGUCA | R |
| WV- | 835 | Mod001L001fUn001RfC*SfG*SfU*SfC*SfG*SfA*SfU*SfG*SfG*SfU* | UCGUCGAUGGUC | OnRSSSSSSSSSSSSS |
| 39810 | | SfC*SfA*SfG*SfC*SmA*SmC*SmA*SmG*SmC*SmC*SmU*SmU*SA* | AGCACAGCCUUA | SSSSSSSSSSSnSSSnR |
| | | SC*SIn001SmC*SmA*SmCn001RmG | CICACG | |
| WV- | 836 | Mod001L001fUn001RfC*SfU*SfC*SfG*SfU*SfC*SfG*SfA*SfU*SfG* | UCUCGUCGAUGG | OnRSSSSSSSSSSSSS |
| 39811 | | SfG*SfU*SfC*SfA*SmG*SmC*SmA*SmC*SmA*SmG*SmC*SmC*ST* | UCAGCACAGCCT | SSSSSSSSSSSnRSSn |
| | | SrC*SAn001RmU*SmG*SmCn001RmA | CAUGCA | R |
| WV- | 837 | Mod001L001fUn001RfC*SfA*SfG*SfC*SfA*SfC*SfA*SfG*SfC*SfC* | UCAGCACAGCCU | OnRSSSSSSSSSSSSS |
| 39812 | | SfU*SfU*SfA*SfU*SmG*SmC*SmA*SmC*SmG*SmG*SmC*SmC*ST* | UAUGCACGGCCT | SSSSSSSSSSSnSSSnR |
| | | SrC*SIn001SmG*SmA*SmGn001RmA | CIGAGA | |
| WV- | 838 | Mod001L001fCn001RfA*SfG*SfC*SfU*SfU*SfC*SOUSfG*SfU*SfC* | CAGCUUCAGUCC | OnRSSSSSSSSSSSSS |
| 39813 | | SfC*SfC*SfU*SfU*SmU*SmC*SmU*SmC*SmG*SmU*SmC*SmG*SA* | CUUUCUCGUCGA | SSSSSSSSSSSnSSSnR |
| | | SrC*SIn001SmG*SmU*SmCn001RmA | CIGUCA | |
| WV- | 839 | Mod001L001fUn001RfC*SfG*SfU*SC*SfG*SfA*SfU*SfG*SfG*SfU* | UCGUCGAUGGUC | OnRSSSSSSSSSSSSn |
| 40403 | | SfC*SfA*SfGn001RfC*SmAn001RmC*SmA*SmG*SmC*SmC*SmU* | AGCACAGCCUUA | RSnRSSSSSSSSSnSS |
| | | SmU*SfA*SC*SIn001SmC*SfA*SmCn001RmG | CICACG | SnR |
| WV- | 840 | Mod001L001fUn001RfC*SfG*SfU*SfC*SfGn001RfA*SfU*SfG*SfG* | UCGUCGAUGGUC | OnRSSSSnRSSSSSnR |
| 40404 | | SfU*SfCn001RfA*SfGn001RfC*SmAn001RmC*SmA*SmG*SmC*SmC* | AGCACAGCCUUA | SnRSnRSSSSSSSSSn |
| | | SmU*SmU*SfA*SC*SIn001SmC*SfA*SmCn001RmG | CICACG | SSSnR |
| WV- | 841 | Mod001L001fUn001RfC*SfGn001RfU*SfC*SfGn001RfA*SfU*SfU*SfG* | UCGUCGAUGGUC | OnRSnRSSnRSSSSSS |
| 40405 | | SfU*SfC*SfA*SfGn001RfC*SmAn001RmC*SmA*SmG*SmCn001RmC* | AGCACAGCCUUA | SnRSnRSSSnRSSSSS |
| | | SmU*SmU*SfA*SC*SIn001SmC*SfA*SmCn001RmG | CICACG | nSSSnR |
| WV- | 842 | Mod001L001fUn001RfC*SfGn001RfU*SfU*SfUn001RIA*SfU*SfG*SfG* | UCGUCGAUGGUC | OnRSnRSSnRSSSSSn |
| 40406 | | SfU*SfCn001RfA*SfGn001RfC*SmAn001RmC*SmA*SmG*SmCn01RmC* | AGCACAGCCUUA | RSnRSnRSSSnRSSSS |
| | | SmU*SmU*SfA*SC*SIn001SmC*SfA*SmCn001RmG | CICACG | SnSSSnR |
| WV- | 843 | Mod001L001fUn001RfC*SfG*SfU*SfC*SfGn001RfA*SfU*SfGn001RfG* | UCGUCGAUGGUC | OnRSSSSnRSSnRSSn |
| 40407 | | SfU*SfCn001RfA*SfGn001RfC*SmAn001RmC*SmA*SmG*SmC* | AGCACAGCCUUA | RSnRSnRSSSSSSSSS |
| | | SmC*SmU*SmU*SfA*SC*SIn001SmC*SfA*SmCn001RmG | CICACG | nSSSnR |
| WV- | 844 | Mod001L001fUn001RfC*SfGn001RfU*SfU*SfGn001RfA*SfU* | UCGUCGAUGGUC | OnRSnRSSnRSSnRSS |
| 40408 | | SfGn001RfG*SfU*SfCn001RfA*SfGn001RfC*SmAn001RmC*SmA*SmG* | AGCACAGCCUUA | nRSnRSnRSSSnRSSS |
| | | SmCn001RmC*SmU*SmU*SfA*SC*SIn001SmC*SfA*SmCn001RmG | CICACG | SSnSSSnR |
| WV- | 845 | Mod001L001fUn001RfC*SfGn001RfU*SfU*SfGn001RIA*SfU*SfU*SfG* | UCGUCGAUGGUC | OnRSnRSSnRSSSSSS |
| 40409 | | SfU*SfC*SfA*SfGn001RfC*SmAn001RfC*SmA*SmG*SfCn001RmC* | AGCACAGCCUUA | SnRSnRSSSnRSSSSS |
| | | SmU*SmU*SfA*SC*SIn001SmC*SfA*SmCn001RmG | CICACG | nSSSnR |
| WV- | 846 | Mod001L001fUn001RfC*SfGn001RfU*SfU*SfGn001RfA*SfU* | UCGUCGAUGGUC | OnRSnRSSnRSSnRSS |
| 40410 | | SfGn001RfG*SfU*SfCn001RfA*SfGn001RfC*SmAn001RfC*SmA*SmG* | AGCACAGCCUUA | nRSnRSnRSSSnRSSS |
| | | SfCn001RmC*SmU*SmU*SfA*SC*SIn001SmC*SfA*SmCn001RmG | CICACG | SSnSSSnR |
| Wy- | 847 | Mod001L001fUn001RfC*SfG*SfU*SfC*SfG*SfA*SfU*SfG*SfG*SfU* | UCGUCGAUGGUC | OnRSSSSSSSSSSSSn |
| 40411 | | SfC*SfA*SfGn001RfC*SmAn001RmC*SmA*SmG*SmC*SmC*SmU* | AGCACAGCCUUA | RSnRSSSSSSSSSnSS |
| | | SmU*SfA*SC*SIn001SmC*SfA*SmC*SmGn001RmG | CICACGG | SSnR |
| WV- | 848 | Mod001L001fUn001RfC*SfGn001RfU*SfC*SfGn001RfA*SfU* | UCGUCGAUGGUC | OnRSnRSSnRSSnRSS |
| 40412 | | SfGn001RfG*SfU*SfC*SfA*SfGn001RfC*SmAn001RmC*SmA*SmG* | AGCACAGCCUUA | SSnRSnRSSSnRSSSS |
| | | SroCn001RmC*SmU*SmU*SfA*SC*SIn001SmC*SfA*SmC*SmGn001RmG | CICACGG | SnSSSSnR |
| WV- | 849 | Mod001L001fcn001RfU*SfC*SfG*SfU*SfC*SfG*SfA*SfU*SfG*SfG* | CUCGUCGAUGGU | OnRSSSSSSSSSSSSS |
| 40413 | | SfU*SfC*SfA*SfGn001RmC*SmAn001RmC*SmA*SmG*SmC*SmC* | CAGCACAGCCUU | aRSnRSSSSSSSSSNS |
| | | SmU*SMU*SfA*SC*SIn001SmC*SfA*SmC*SmGn001RmG | acicacgg | SSSnR |
| WV- | 850 | Mod001L001fcn001RfU*SfC*SfGn001RfU*SfC*SfGn001RfA*SfU* | CUCGUCGAUGGU | OnRSSnRSSnRSSnRS |
| 40414 | | sFGn001RfG*SfU*SfC*SfA*SfGn001RmC*SmAn001RmC*SmA*SmG* | CAGCACAGCCUU | SSSnRSnRSSSnRSSS |
| | | SmCn001RmC*SmU*SmU*SfA*SC*SIn001SmC*SfA*SmC*SmGn001RmG | ACICACGG | SSnSSSSnR |
| WV- | 851 | Mod001L0011Un001RfC*SfU*SfC*SfG*SfU*SfC*SfG*SfA*SfU*SfG* | UCUCGUCGAUGG | OnRSSSSSSSSSSSSn |
| 40438 | | SfG*SfU*SfCn001RfA*SmGn001RmC*SmA*SmC*SmA*SmG*SmC* | UCAGCACAGCCU | RSnRSSSSSSSSSnRS |
| | | SmC*SfU*SC*SAn001RmU*SfG*SmCn001RmA | CAUGCA | SnR |
| WV- | 852 | Mod001L001fUn001RfC*SfU*SfC*SfG*SfU*SfC*SfG*SfA*SfU*SfG* | UCUCGUCGAUGG | OnRSSSSSSSSSSSSn |
| 40438 | | SfG*SfU*SfCn001RfA*SmGn001RmC*SmA*SmC*SmA*SmG*SmC* | UCAGCACAGCCU | RSnRSSSSSSSSSnRS |
| | | SmC*SfU*SC*SAn001RmU*SfG*SmCn001RmA | CAUGCA | SnR |
| WV- | 853 | Mod001L001fUn001RfC*SfU*SfC*SfG*SfUn001RfC*SfG*SfA*SfU* | UCUCGUCGAUGG | OnRSSSSnRSSSSSnR |
| 40439 | | SfG*SfGn001RfU*SfCn001RfA*SmGn001RmC*SmA*SmC*SmA*SmG* | UCAGCACAGCCU | SnRSnRSSSSSSSSSn |
| | | SmC*SmC*SfU*SC*SAn001RmU*SfG*SmCn001RmA | CAUGCA | RSSnR |
| WV- | 854 | Mod001L00lfUn001RfC*SfUn001RfC*SfG*SfUn001RfC*SfG*SfA*SfU* | UCUCGUCGAUGG | OnRSnRSSnRSSSSSS |
| 40440 | | SfG*SfG*SfU*SfCn001RfA*SmGn001RmC*SmA*SmC*SmAn001RmG* | UCAGCACAGCCU | SnRSnRSSSnRSSSSS |
| | | SmC*SmC*SfU*SC*SAn001RmU*SfG*SmCn001RmA | CAUGCA | nRSSnR |
| WV- | 855 | Mod001L001fUn001RfC*SfUn001RfC*SfG*SfUn001RfC*SfG*SfA*SfU* | UCUCGUCGAUGG | OnRSnRSSnRSSSSSn |
| 40441 | | SfG*SfGn001RfU*SfCn001RfA*SmGn001RmC*SmA*SmC*SmAn001RmG* | UCAGCACAGCCU | RSnRSnRSSSnRSSSS |
| | | SmC*SmC*SfU*SC*SAn001RmU*SfG*SmCn001RmA | CAUGCA | SnRSSnR |
| WV- | 856 | Mod001L001fUn001RfC*SfU*SfC*SfG*SfUn001RfC*SfG*SfAn001RfU* | UCUCGUCGAUGG | OnRSSSSnRSSnRSSn |
| 40442 | | SfG*SfGn001RfU*SfCn001RfA*SmGn001RmC*SmA*SmC*SmA* | UCAGCACAGCCU | RSnRSnRSSSSSSSSS |
| | | SmG*SmC*SmC*SfU*SC*SAn001RmU*SfG*SmCn001RmA | CAUGCA | nRSSnR |
| WV- | 857 | Mod001L001fUn001RfC*SfUn001RfC*SfG*SfUn001RfC*SfG* | UCUCGUCGAUGG | OnRSnRSSnRSSnRSS |
| 40443 | | SfAn00RfU*SfG*SfGn001RfU*SfCn001RfA*SmGn001RmC*SmA*SmC* | UCAGCACAGCCU | nRSnRSnRSSSnRSSS |
| | | SmAn001RmG*SmC*SmC*SfU*SC*SAn001RmU*SfG*SmCn001RmA | CAUGCA | SSnRSSnR |
| WV- | 858 | Mod001L001fUn001RfC*SfUn001RfC*SfG*SfUn001RfC*SfG*SfA*SfU* | UCUCGUCGAUGG | OnRSnRSSnRSSSSSS |
| 40444 | | SfG*SfG*SfU*SfCn001RfA*SmGn001RfC*SmA*SmC*SfAn001RmG* | UCAGCACAGCCU | SnRSnRSSSnRSSSSS |
| | | SmC*SmC*SfU*SC*SAn001RmU*SfG*SmCn001RmA | CAUGCA | nRSSnR |
| WV- | 859 | Mod001L001fUn001RfC*SfUn001RfC*SfG*SfUn001RfC*SfG* | UCUCGUCGAUGG | OnRSnRSSnRSSnRSS |
| 40445 | | SfAn001RfU*SfG*SfGn001RfU*SfCn001RfA*SmGn001RfC*SmA*SmC* | UCAGCACAGCCU | nRSnRSnRSSSnRSSS |
| | | SfAn001RmG*SmC*SmC*SfU*SC*SAn001RmU*SfG*SmCn001RmA | CAUGCA | SSnRSSnR |
| WV- | 860 | Mod001L001fUn001RfC*SfU*SfC*Sf*SfU*SfC*SfG*SfA*SfU*SfG* | UCUCGUCGAUGG | OnRSSSSSSSSSSSSn |
| 40446 | | SfG*SfU*SfCn001RfA*SmGn001RmC*SmA*SmC*SmA*SmG*SmC* | UCAGCACAGCCU | RSnRSSSSSSSSSnRS |
| | | SmC*SfU*SC*SAn001RmU*SfG*SmC*SmAn001RmC | CAUGCAC | SSnR |
| WV- | 861 | Mod001L001fUn001RfC*SfU*SfC*SfG*SfU*SfC*SfG*SfA*SfU*SfG* | UCUCGUCGAUGG | OnRSSSSSSSSSSSSn |
| 40446 | | SfG*SfU*SfCn001RfA*SmGn001RmC*SmA*SmC*SmA*SmG*SmC* | UCAGCACAGCCU | RSnRSSSSSSSSSnRS |
| | | SmC*SfU*SC*SAn001RmU*SfG*SmC*SmAn001RmC | CAUGCAC | SSnR |
| WV- | 862 | Mod001L001fUn001RfC*SfUn001RfC*SfG*SfUn001RfC*SfG* | UCUCGUCGAUGG | OnRSnRSSnRSSnRSS |
| 40447 | | SfAn001RfU*SfG*SfG*SfU*SfCn001RfA*SmGn001RmC*SmA*SmC* | UCAGCACAGCCU | SSnRSnRSSSnRSSSS |
| | | SmAn001RmG*SmC*SmC*SfU*SC*SAn001RmU*SfG*SmC*SmAn001RmC | CAUGCAC | SnRSSSnR |
| WV- | 863 | Mod001L001fUn001RfU*SfC*SfU*SfC*SfG*SfU*SC*SfG*SfA*SfU* | UUCUCGUCGAUG | OnRSSSSSSSSSSSSS |
| 40448 | | SfG*SfG*SfU*SfCn001RmA*SmGn001RmC*SmA*SmC*SmA*SmG* | GUCAGCACAGCC | aRSnRSSSSSSSSSnR |
| | | SmC*SmC*SfU*SC*SAn001RmU*SfG*SmC*SmAn001RmC | UCAUGCAC | SSSnR |
| WV- | 864 | Mod001L001fUn001RfU*SfC*SfUn001RfC*SfG*SfUn001RfC*SfG* | UUCUCGUCGAUG | OnRSSnRSSnRSSnRS |
| 40449 | | SfAn001RfU*SfG*SfG*SfU*SfCn001RmA*SmGn001RmC*SmA*SmC* | GUCAGCACAGCC | SSSnRSnRSSSnRSSS |
| | | SmAn001RmG*SmC*SmC*SfU*SC*SAn001RmU*SfG″SmC*SmAn001RnC | UCAUGCAC | SSnRSSSnR |
| WV- | 865 | Mod001L001fUn001RfC*SfA*SfU*SfC*SfG*SfA*SfU*SfG*SfG*SfU* | UCAUCGAUGGUC | OnRSSSSSSSSSSSSn |
| 40798 | | SfC*SfA*SfGn001RfC*SmAn001RmC*SmA*SmG*SmC*SmC*SmU* | AGCACAGCCUUA | RSnRSSSSSSSSSnSS |
| | | SmU*SfA*SC*SIn001SmC*SfA*SmCn001RmG | CICACG | SnR |
| WV- | 866 | Mod001L001fUn001RfC*SfU*SfC*SfA*SfU*SfC*SfG*SfA*SfU*SfG* | UCUCAUCGAUGG | OnRSSSSSSSSSSSSn |
| 40799 | | SfG*SfU*SfCn001RfA*SmGn001RmC*SmA*SmC*SmA*SmG*SmC* | UCAGCACAGCCU | RSnRSSSSSSSSSnRS |
| | | SmC*SfU*SC*SAn001RmU*SfG*SmCn001RmA | CAUGCA | SnR |
| WV- | 867 | Mod001L001fUn001RfC*SfUn001RfC*SfA*SfUn001RfC*SfG*SfA*SfU* | UCUCAUCGAUGG | OnRSnRSSnRSSSSSS |
| 40829 | | SfG*SfG*SfU*SfCn001RfA*SmGn001RfC*SmA*SmC*SfAn001RmG* | UCAGCACAGCCU | SnRSnRSSSnRSSSSS |
| | | SmC*SmC*SfU*SC*SAn001RmU*SfG*SmCn001RmA | CAUGCA | nRSSnR |
| WV- | 868 | Mod001L001*fUn001RfC*SfG*SfU*SA*SfGn001RfA*SfU*SfG*SfG* | UCGUCGAUGGUC | XnRSSSSnRSSSSSnR |
| 41194 | | SfU*SfUn001RfA*SfGn001RfC*SmAn001RmC*SmA*SmG*SmC*SmC* | AGCACAGCCUUA | SnRSnRSSSSSSSSSn |
| | | SmU*SmU*SfA*SC*SIn001SmC*SfA*SmCn001RmG | CICACG | SSSnR |

TABLE 1I
Example oligonucleotides and/or compositions that target UGP2.

| | SEQ | | | Stereochemistry/ |
|---|---|---|---|---|
| ID | ID NO | Description | Base Sequence | Linkage |
| WV- | 869 | fAn001RfU*SfC*SfC*SfA*SfC*SfU*SfG*SfU*SfG*SfG*SfC*SfA* | AUCCACUGUGGC | nRSSSSSSSSSSSSnRSn |
| 40590 | | Sfn001RfC*Smcn001RmA*SmG*SmA*SmU*SmU*SmA*SmU*SfC* | ACCCAGAUUAUC | RSSSSSSSSSnRSSnR |
| | | SC*SAn001RmU*SmG*SmUn001RmU | CAUGUU | |

### Notes:

Description, Base Sequence and Stereochemistry/Linkage, due to their length, may be divided into multiple lines in Table 1 (e.g., Table 1A, Table 1B, Table 1C, etc.). Unless otherwise specified, all oligonucleotides in Table 1 are single-stranded. As appreciated by those skilled in the art, nucleoside units are unmodified and contain unmodified nucleobases and 2′-deoxy sugars unless otherwise indicated (e.g., with r, m, m5, eo, etc.); linkages, unless otherwise indicated, are natural phosphate linkages; and acidic/basic groups may independently exist in their salt forms. If a sugar is not specified, the sugar is a natural DNA sugar; and if an internucleotidic linkage is not specified, the internucleotidic linkage is a natural phosphate linkage. Moieties and modifications:

### m: 2′-OMe;

m5: methyl at 5-position of C (nucleobase is 5-methylcytosine);
m5lC: methyl at 5-position of C (nucleobase is 5-methylcytosine) and sugar is a LNA sugar; l: LNA sugar;
I: nucleobase is hypoxanthine;

### f: 2′-F;

### r: 2′-OH;

eo: 2′-MOE (2′—OCH₂CH₂OCH₃);
m5Ceo: 5-methyl 2′-O-methoxyethyl C;
O, PO: phosphodiester (phosphate). It can a linkage or be an end group (or a component thereof), e.g., a linkage between a linker and an oligonucleotide chain, an internucleotidic linkage (a natural phosphate linkage), etc. Phosphodiesters are typically indicated with “0” in the Stereochemistry/Linkage column and are typically not marked in the Description column (if it is an end group, e.g., a 5′-end group, it is indicated in the Description and typically not in Stereochemistry/Linkage); if no linkage is indicated in the Description column, it is typically a phosphodiester unless otherwise indicated. Note that a phosphate linkage between a linker (e.g., L001) and an oligonucleotide chain may not be marked in the Description column, but may be indicated with “O” in the Stereochemistry/Linkage column;
*, PS: Phosphorothioate. It can be an end group (if it is an end group, e.g., a 5′-end group, it is indicated in the Description and typically not in Stereochemistry/Linkage), or a linkage, e.g., a linkage between linker (e.g., L001) and an oligonucleotide chain, an internucleotidic linkage (a phosphorothioate internucleotidic linkage), etc.;
R, Rp: Phosphorothioate in the Rp configuration. Note that *R in Description indicates a single phosphorothioate linkage in the Rp configuration;
S, Sp: Phosphorothioate in the Sp configuration. Note that *S in Description indicates a single phosphorothioate linkage in the Sp configuration;
X: stereorandom phosphorothioate;
n001:

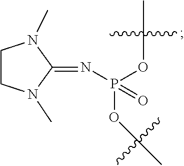 [SMILES: *OP(=O)(N=C1N(C)CCN1C)O*]

nX: stereorandom n001;
nR or n001R: n001 in Rp configuration;
nS or n001S: n001 in Sp configuration;

### Mod001:

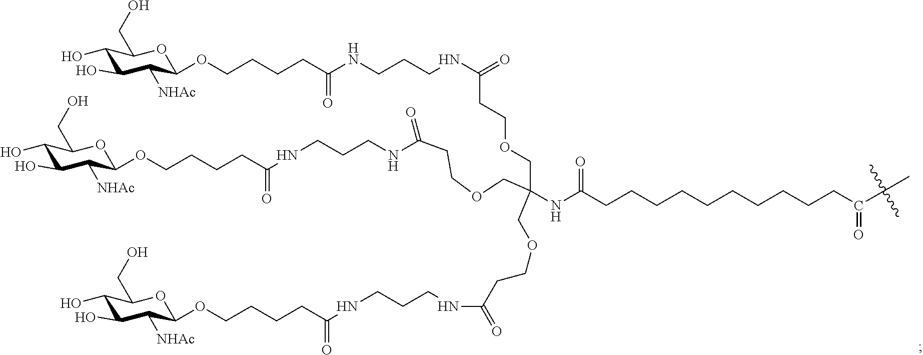

L001: —NH—(CH₂)₆— linker (C6 linker, C6 amine linker or C6 amino linker), connected to Mod (e.g., Mod001) through —NH—, and, in the case of, for example, WV-27457, the 5′-end of the oligonucleotide chain through a phosphate linkage (O or PO). For example, in WV-27457, L001 is connected to Mod001 through —NH— (forming an amide group —C(O)—NH—), and is connected to the oligonucleotide chain through a phosphate linkage (O);

### L010:

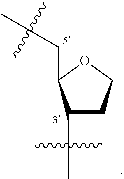

In some embodiments, when L010 is present in the middle of an oligonucleotide, it is bonded to internucleotidic linkages as other sugars (e.g., DNA sugars), e.g., its 5′-carbon is connected to another unit (e.g., 3′ of a sugar) and its 3′-carbon is connected to another unit (e.g., a 5′-carbon of a carbon) independently, e.g., via a linkage (e.g., a phosphate linkage (O or PO) or a phosphorothioate linkage (can be either not chirally controlled or chirally controlled (Sp or Rp)));
L012:—CH₂CH₂OCH₂CH₂OCH₂CH₂—. When L012 is present in the middle of an oligonucleotide, each of its two ends is independently bonded to an internucleotidic linkage (e.g., a phosphate linkage (O or PO) or a phosphorothioate linkage (can be either not chirally controlled or chirally controlled (Sp or Rp)));

### L025:

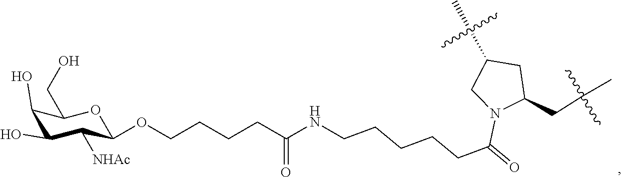

wherein the —CH₂— connection site is utilized as a C5 connection site of a sugar (e.g., a DNA sugar) and is connected to another unit (e.g., 3′ of a sugar), and the connection site on the ring is utilized as a C3 connection site and is connected to another unit (e.g., a 5′-carbon of a carbon), each of which is independently, e.g., via a linkage (e.g., a phosphate linkage (O or PO) or a phosphorothioate linkage (can be either not chirally controlled or chirally controlled (Sp or Rp))). When L025 is at a5′-end without any modifications, its —CH₂— connection site is bonded to —OH. For example, L025L025L025— in various oligonucleotides has the structure of

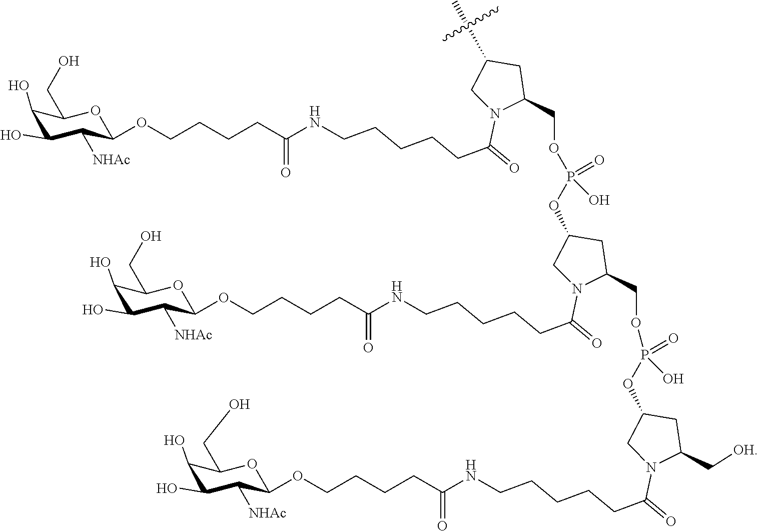

(may exist as various salt forms) and is connected to 5′-carbon of an oligonucleotide chain via a linkage as indicated (e.g., a phosphate linkage (O or PO) or a phosphorothioate linkage (can be either not chirally controlled or chirally controlled (Sp or Rp)));
L028:—CH₂CH₂OCH₂CH₂OCH₂CH₂OCH₂CH₂—. When L028 is present in the middle of an oligonucleotide, each of its two ends is independently bonded to an internucleotidic linkage (e.g., a phosphate linkage (O or PO) or a phosphorothioate linkage (can be either not chirally controlled or chirally controlled (Sp or Rp)));
connection site on the ring is utilized as a C3 connection site of a sugar (e.g., a DNA sugar), each of which is independently bonded to
sm04:

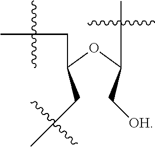

sm04 follows a nucleobase to which it is bound; for example, in WV-28787, for example, “Usm04” indicates that U is bonded to sm04

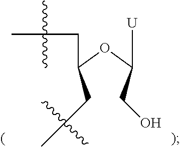

### a: 2′-NH₂;

b001U: a nucleoside whose base is

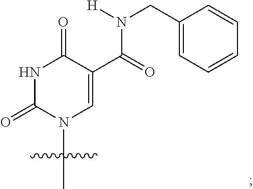 [SMILES: *n1cc(C(=O)NCc2ccccc2)c(=O)[nH]c1=O]

b001rU: a nucleoside whose base is

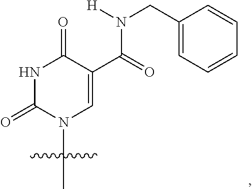 [SMILES: *n1cc(C(=O)NCc2ccccc2)c(=O)[nH]c1=O]

and whose sugar is a natural RNA sugar (r);
b002U: a nucleoside whose base is

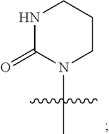 [SMILES: *N1CCCNC1=O]

b003U: a nucleoside whose base is

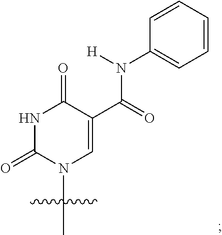 [SMILES: *n1cc(C(=O)Nc2ccccc2)c(=O)[nH]c1=O]

b004U: a nucleoside whose base is

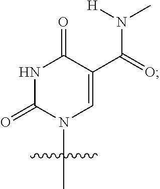 [SMILES: *n1cc(C(=O)NC)c(=O)[nH]c1=O]

b005U: a nucleoside whose base is

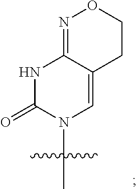 [SMILES: *N1C=C2CCON=C2NC1=O]

b006U: a nucleoside whose base is

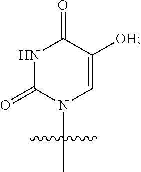 [SMILES: *n1cc(O)c(=O)[nH]c1=O]

b007U: a nucleoside whose base is

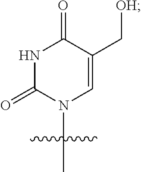 [SMILES: *n1cc(CO)c(=O)[nH]c1=O]

b008U: a nucleoside whose base is

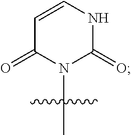 [SMILES: *n1c(=O)cc[nH]c1=O]

b009U: a nucleoside whose base is

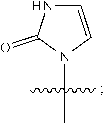 [SMILES: *n1cc[nH]c1=O]

b0031: a nucleoside whose base is

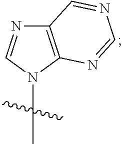 [SMILES: *n1cnc2cncnc21]

b001G: a nucleoside whose base is

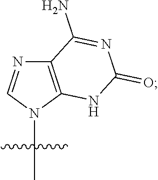 [SMILES: *n1cnc2c(N)nc(=O)[nH]c21]

b001A: a nucleoside whose base is

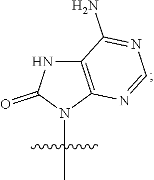 [SMILES: *n1c(=O)[nH]c2c(N)ncnc21]

b002A: a nucleoside whose base is

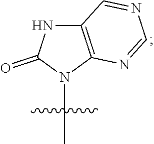 [SMILES: *n1c(=O)[nH]c2cncnc21]

b003A: a nucleoside whose base is

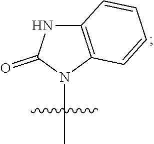 [SMILES: *n1c(=O)[nH]c2ccccc21]

zdnp: a nucleoside whose base is

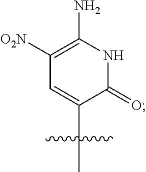 [SMILES: *c1cc([N+](=O)[O-])c(N)[nH]c1=O]

b001C: a nucleoside whose base is

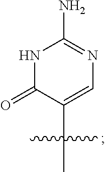 [SMILES: *c1cnc(N)[nH]c1=O]

b002C: a nucleoside whose base is

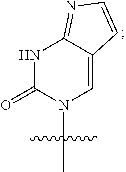 [SMILES: *n1cc2ccnc-2[nH]c1=O]

b003C: a nucleoside whose base is

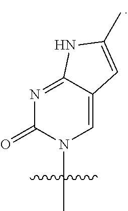 [SMILES: *n1cc2cc(C)[nH]c2nc1=O]

### Oligonucleotide Compositions

Among other things, the present disclosure provides various oligonucleotide compositions. In some embodiments, the present disclosure provides oligonucleotide compositions of oligonucleotides described herein. In some embodiments, an oligonucleotide composition comprises a plurality of oligonucleotides described in the present disclosure. In some embodiments, an oligonucleotide composition is chirally controlled. In some embodiments, an oligonucleotide composition is not chirally controlled (stereorandom).

Linkage phosphorus of natural phosphate linkages is achiral. Linkage phosphorus of many modified internucleotidic linkages, e.g., phosphorothioate internucleotidic linkages, are chiral. In some embodiments, during preparation of oligonucleotide compositions (e.g., in traditional phosphoramidite oligonucleotide synthesis), configurations of chiral linkage phosphorus are not purposefully designed or controlled, creating non-chirally controlled (stereorandom) oligonucleotide compositions (substantially racemic preparations) which are complex, random mixtures of various stereoisomers (diastereoisomers) —for oligonucleotides with n chiral internucleotidic linkages (linkage phosphorus being chiral), typically 2n stereoisomers (e.g., when n is 10, 210=1,032; when n is 20, 220=1,048,576). These stereoisomers have the same constitution, but differ with respect to the pattern of stereochemistry of their linkage phosphorus.

In some embodiments, stereorandom oligonucleotide compositions have sufficient properties and/or activities for certain purposes and/or applications. In some embodiments, stereorandom oligonucleotide compositions can be cheaper, easier and/or simpler to produce than chirally controlled oligonucleotide compositions. However, stereoisomers within stereorandom compositions may have different properties, activities, and/or toxicities, resulting in inconsistent therapeutic effects and/or unintended side effects by stereorandom compositions, particularly compared to certain chirally controlled oligonucleotide compositions of oligonucleotides of the same constitution.

In some embodiments, the present disclosure encompasses technologies for designing and preparing chirally controlled oligonucleotide compositions. In some embodiments, the present disclosure provides chirally controlled oligonucleotide compositions, e.g., of many oligonucleotides in Table 1 which contain S and/or R in their stereochemistry/linkage. In some embodiments, a chirally controlled oligonucleotide composition comprises a controlled/pre-determined (not random as in stereorandom compositions) level of a plurality of oligonucleotides, wherein the oligonucleotides share the same linkage phosphorus stereochemistry at one or more chiral internucleotidic linkages (chirally controlled internucleotidic linkages). In some embodiments, the oligonucleotides share the same pattern of backbone chiral centers (stereochemistry of linkage phosphorus). In some embodiments, a pattern of backbone chiral centers is as described in the present disclosure. In some embodiments, oligonucleotides of a plurality are structural identical.

In some embodiments, the present disclosure provides an oligonucleotide composition comprising a plurality of oligonucleotides, wherein oligonucleotides of the plurality share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”).

In some embodiments, the present disclosure provides an oligonucleotide composition comprising a plurality of oligonucleotides, wherein oligonucleotides of the plurality share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein the composition is enriched, relative to a substantially racemic preparation of oligonucleotides sharing the common base sequence, for oligonucleotides of the plurality.

In some embodiments, an oligonucleotide composition is a chirally controlled oligonucleotide composition comprising a plurality of oligonucleotides, wherein the oligonucleotides share:

a common base sequence,

a common pattern of backbone linkages, and

the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),

wherein the composition is enriched, relative to a substantially racemic preparation of oligonucleotides sharing the common base sequence and pattern of backbone linkages, for oligonucleotides of the plurality.

In some embodiments, an oligonucleotide composition is a chirally controlled oligonucleotide composition comprising a plurality of oligonucleotides, wherein the oligonucleotides share:

a common base sequence,

a common patter of backbone linkages, and

a common pattern of backbone chiral centers, which pattern comprises at least one Sp,

wherein the composition is enriched, relative to a substantially racemic preparation of oligonucleotides sharing the common base sequence and pattern of backbone linkages, for oligonucleotides of the plurality.

In some embodiments, an oligonucleotide composition is a chirally controlled oligonucleotide composition comprising a plurality of oligonucleotides, wherein the oligonucleotides share:

a common base sequence,

a common patter of backbone linkages, and

a common pattern of backbone chiral centers, which pattern comprises at least one Rp,

wherein the composition is enriched, relative to a substantially racemic preparation of oligonucleotides sharing the common base sequence and pattern of backbone linkages, for oligonucleotides of the plurality.

In some embodiments, the present disclosure provides a chirally controlled oligonucleotide composition comprising a plurality of oligonucleotides, wherein the oligonucleotides share:

1) a common constitution, and

2) share the same linkage phosphorus stereochemistry at one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages (chirally controlled internucleotidic linkages),

wherein the composition is enriched, relative to a substantially racemic preparation of oligonucleotides of the common constitution, for oligonucleotides of the plurality.

In some embodiments, the present disclosure provides an oligonucleotide composition comprising a plurality of oligonucleotides, wherein oligonucleotides of the plurality share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein stereochemical purity of the linkage phosphorus of each chirally controlled internucleotidic linkage is independently 80%-100% (e.g., 85-100%, 90-100%, about or at least about 90%, 91%, 92%, 93%, 94%, 95%, 96%, 97%, 98%, 99% or 99.5%).

In some embodiments, an oligonucleotide composition is a chirally controlled oligonucleotide composition comprising a plurality of oligonucleotides, wherein the oligonucleotides share:

a common base sequence,

a common pattern of backbone linkages, and

the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),

wherein stereochemical purity of the linkage phosphorus of each chirally controlled internucleotidic linkage is independently 80%-100% (e.g., 85-100%, 90-100%, about or at least about 90%, 91%, 92%, 93%, 94%, 95%, 96%, 97%, 98%, 99% or 99.5%).

In some embodiments, the present disclosure provides a chirally controlled oligonucleotide composition comprising a plurality of oligonucleotides, wherein the oligonucleotides share:

1) a common constitution, and

2) share the same linkage phosphorus stereochemistry at one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages (chirally controlled internucleotidic linkages),

wherein stereochemical purity of the linkage phosphorus of each chirally controlled internucleotidic linkage is independently 80%-100% (e.g., 85-100%, 90-100%, about or at least about 90%, 91%, 92%, 93%, 94%, 95%, 96%, 97%, 98%, 99% or 99.5%).

In some embodiments, the present disclosure provides an oligonucleotide composition comprising a plurality of oligonucleotides, wherein oligonucleotides of the plurality share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein the common base sequence is complementary to a base sequence of a portion of a nucleic acid which portion comprises a target adenosine.

In some embodiments, the present disclosure provides an oligonucleotide composition comprising one or more pluralities of oligonucleotides, wherein oligonucleotides of each plurality independently share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein the common base sequence of each plurality is independently complementary to a base sequence of a portion of a nucleic acid which portion comprises a target adenosine.

In some embodiments, the present disclosure provides an composition comprising a plurality of oligonucleotides which are of a particular oligonucleotide type characterized by:

a) a common base sequence;

b) a common pattern of backbone linkages;

c) a common pattern of backbone chiral centers;

d) a common pattern of backbone phosphorus modifications;

which composition is chirally controlled in that it is enriched, relative to a substantially racemic preparation of oligonucleotides having the same common base sequence, pattern of backbone linkages and pattern of backbone phosphorus modifications, for oligonucleotides of the particular oligonucleotide type, or a non-random level of all oligonucleotides in the composition that share the common base sequence are oligonucleotides of the plurality; and

wherein the common base sequence is complementary to a base sequence of a portion of a nucleic acid which portion comprises a target adenosine.

In some embodiments, as described herein a portion can be about or at least about 10-40, 15-40, 20-40, e.g., 10, 11, 12, 13, 14, 15, 16, 17, 18, 19, 20, 21, 22, 23, 24, 25, 26, 27, 28, 29 or 30 or more, nucleobases long. In some embodiments, a portion is about or at least about or no more than about 1%-50% of a nucleic acid. In some embodiments, a portion is the whole length of a nucleic acid. In some embodiments, a common base sequence is complementary to a base sequence of a portion of a nucleic acid as described herein. In some embodiments, it is fully complementary across its length except at a nucleobase opposite to a target adenosine. In some embodiments, it is fully complementary across its length. In some embodiments, a target adenosine is associated with a condition, disorder or disease. In some embodiments, a target adenosine is a G to A mutation associated with a condition, disorder or disease. In some embodiments, a target adenosine is edited to I by a provided oligonucleotide or composition. In some embodiments, as described herein editing increases expression, level and/or activity of a transcript or a product thereof (e.g., a mRNA, a protein, etc.). In some embodiments, as described herein editing reduces expression, level and/or activity of a transcript or a product thereof (e.g., a mRNA, a protein, etc.).

In some embodiments, oligonucleotide of a plurality share the same nucleobase modifications and/or sugar modifications. In some embodiments, oligonucleotide of a plurality share the same internucleotidic linkage modifications (wherein the internucleotidic linkages may be in various acid, base, and/or salt forms). In some embodiments, oligonucleotides of a plurality share the same nucleobase modifications, sugar modifications, and internucleotidic linkage modifications, if any. In some embodiments, oligonucleotides of a plurality are of the same form, e.g., an acid form, a base form, or a particularly salt form (e.g., a pharmaceutically acceptable salt form, e.g., salt form). In some embodiments, oligonucleotides in a composition may exist as one or more forms, e.g., acid forms, base forms, and/or one or more salt forms. In some embodiments, in an aqueous solution (e.g., when dissolved in a buffer like PBS), anions and cations may dissociate. In some embodiments, oligonucleotides of a plurality are of the same constitution. In some embodiments, oligonucleotides of a plurality are structurally identical. In some embodiments, the present disclosure provides a chirally controlled oligonucleotide composition comprising a plurality of oligonucleotides, wherein the oligonucleotides are of a common constitution, and share the same linkage phosphorus stereochemistry at one or more (e.g., 1-60, 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more) chiral internucleotidic linkages (chirally controlled internucleotidic linkages), wherein the composition is enriched, relative to a substantially racemic preparation of oligonucleotides of the common constitution, for oligonucleotides of the plurality.

In some embodiments, at least one chiral internucleotidic linkage is chirally controlled. In some embodiments, at least 2 internucleotidic linkages are independently chirally controlled. In some embodiments, the number of chirally controlled internucleotidic linkages is at least 3. In some embodiments, it is at least 4. In some embodiments, it is at least 5. In some embodiments, it is at least 6. In some embodiments, it is at least 7. In some embodiments, it is at least 8. In some embodiments, it is at least 9. In some embodiments, it is at least 10. In some embodiments, it is at least 11. In some embodiments, it is at least 12. In some embodiments, it is at least 13. In some embodiments, it is at least 14. In some embodiments, it is at least 15. In some embodiments, it is at least 20. In some embodiments, it is at least 25. In some embodiments, it is at least 30.

In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all internucleotidic linkages are chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all chiral internucleotidic linkages are chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all phosphorothioate internucleotidic linkages are chirally controlled. In some embodiments, a percentage is at least 50%. In some embodiments, a percentage is at least 60%. In some embodiments, a percentage is at least 70%. In some embodiments, a percentage is at least 80%. In some embodiments, a percentage is at least 90%. In some embodiments, a percentage is at least 90%. In some embodiments, each chiral internucleotidic linkage is chirally controlled. In some embodiments, each phosphorothioate internucleotidic linkage is chirally controlled.

In some embodiments, no more than 1-10, e.g., no more than 1, 2, 3, 4, 5, 6, 7, 8, 9, or 10, chiral internucleotidic linkages are not chirally controlled. In some embodiments, no more than 1 chiral internucleotidic linkages is not chirally controlled. In some embodiments, no more than 2 chiral internucleotidic linkages are not chirally controlled. In some embodiments, no more than 3 chiral internucleotidic linkages are not chirally controlled. In some embodiments, no more than 4 chiral internucleotidic linkages are not chirally controlled. In some embodiments, no more than 5 chiral internucleotidic linkages are not chirally controlled. In some embodiments, the number of non-chirally controlled internucleotidic linkages is 1. In some embodiments, it is 2. In some embodiments, it is 3. In some embodiments, it is 4. In some embodiments, it is 5.

In some embodiments, an enrichment relative to a substantially racemic preparation is that at least about 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition, or all oligonucleotides in the composition that share the common base sequence of a plurality, or all oligonucleotides in the composition that share the common constitution of a plurality, are oligonucleotide of the plurality. In some embodiments, a percentage is at least 10%. In some embodiments, a percentage is at least 20%. In some embodiments, a percentage is at least 30%. In some embodiments, a percentage is at least 40%. In some embodiments, a percentage is at least 50%. In some embodiments, it is at least 60%. In some embodiments, it is at least 70%. In some embodiments, it is at least 80%. In some embodiments, it is at least 90%. In some embodiments, it is at least 95%.

Levels of oligonucleotides of a plurality in chirally controlled oligonucleotide compositions are controlled. In contrast, in non-chirally controlled (or stereorandom, racemic) oligonucleotide compositions (or preparations), levels of oligonucleotides are random and not controlled. In some embodiments, an enrichment relative to a substantially racemic preparation is a level described herein.

In some embodiments, a level as a percentage (e.g., a controlled level, a pre-determined level, an enrichment) is or is at least (DS)^{nc}, wherein DS (diastereopurity of an individual internucleotidic linkage) is 90%-100%, and nc is the number of chirally controlled internucleotidic linkages as described in the present disclosure (e.g., 5, 6, 7, 8, 9, 10, 11, 12, 13, 14, 15, 16, 17, 18, 19, 20 or more). In some embodiments, each chiral internucleotidic linkage is chirally controlled, and nc is the number of chiral internucleotidic linkage. In some embodiments, DS is 90%, 91%, 92%, 93%, 94%, 95%, 96%, 97%, 98%, 99% or 99.5% or more. In some embodiments, DS is or is at least 90%. In some embodiments, DS is or is at least 91%. In some embodiments, DS is or is at least 92%. In some embodiments, DS is or is at least 93%. In some embodiments, DS is or is at least 94%. In some embodiments, DS is or is at least 95%. In some embodiments, DS is or is at least 96%. In some embodiments, DS is or is at least 97%. In some embodiments, DS is or is at least 98%. In some embodiments, DS is or is at least 99%. In some embodiments, a level (e.g., a controlled level, a pre-determined level, an enrichment) is a percentage of all oligonucleotides in a composition that share the same constitution, wherein the percentage is or is at least (DS)^{nc}. For example, when DS is 99% and nc is 10, the percentage is or is at least 90% ((99%)¹⁰≈0.90=90%). As appreciated by those skilled in the art, in a stereorandom preparation the percentage is typically about ½^{nc}— when nc is 10, the percentage is about ½¹⁰≈0.001=0.1%. In some embodiments, an enrichment (e.g., relative to a substantially racemic preparation), a level, etc., is that at least about (DS)^{nc} of all oligonucleotides in the composition, or all oligonucleotides in the composition that share the common base sequence of a plurality, or all oligonucleotides in the composition that share the common constitution of a plurality, are oligonucleotide of the plurality. In some embodiments, it is of all oligonucleotides in the composition. In some embodiments, it is of all oligonucleotides in the composition that share the common base sequence of a plurality. In some embodiments, it is of all oligonucleotides in the composition that share the common constitution of a plurality. In some embodiments, various forms (e.g., various salt forms) of an oligonucleotide may be properly considered to have the same constitution.

In some embodiments, an oligonucleotide composition (also referred to as an oligonucleotide composition) is a chirally controlled oligonucleotide composition comprising a plurality of oligonucleotides, wherein the oligonucleotides share:

a common base sequence,

a common pattern of backbone linkages, and

the same linkage phosphorus stereochemistry at one or more chiral internucleotidic linkages (chirally controlled internucleotidic linkages),

wherein the percentage of the oligonucleotides of the plurality within all oligonucleotides in the composition that share the common base sequence and pattern of backbone linkages is at least (DS)^{nc}, wherein DS is 90%-100%, and nc is the number of chirally controlled internucleotidic linkages.

In some embodiments, an oligonucleotide composition (also referred to as an oligonucleotide composition) is a chirally controlled oligonucleotide composition comprising a plurality of oligonucleotides, wherein the oligonucleotides share:

a common base sequence,

a common patter of backbone linkages, and

a common pattern of backbone chiral centers, which pattern comprises at least one Sp,

wherein the percentage of the oligonucleotides of the plurality within all oligonucleotides in the composition that share the common base sequence and pattern of backbone linkages is at least (DS)^{nc}, wherein DS is 90%-100%, and nc is the number of chirally controlled internucleotidic linkages.

In some embodiments, level of a diastereopurity of a plurality of oligonucleotides in a composition can be determined as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy).

In some embodiments, a chirally controlled oligonucleotide composition comprises two or more pluralities of oligonucleotides, wherein each plurality is independently a plurality of oligonucleotides as described herein (e.g., in various chirally controlled oligonucleotide compositions). For example, in some embodiments, each plurality independently shares a common base sequence, and the same linkage phosphorus stereochemistry at one or more chiral internucleotidic linkages, and each plurality is independently enriched compared to stereorandom preparation of that plurality or each plurality is independently of a level as described herein. In some embodiments, at least two pluralities or each plurality independently targets a different adenosine. In some embodiments, at least two pluralities or each plurality independently targets a different transcript of the same or different nucleic acids. In some embodiments, at least two pluralities or each plurality independently targets transcripts of a different gene. Among other things, such compositions may be utilized to target two or more targets, in some embodiments, simultaneously and in the same system.

In some embodiments, all chiral internucleotidic linkages are chiral controlled, and the composition is a completely chirally controlled oligonucleotide composition. In some embodiments, not all chiral internucleotidic linkages are chiral controlled internucleotidic linkages, and the composition is a partially chirally controlled oligonucleotide composition.

Oligonucleotides may comprise or consist of various patterns of backbone chiral centers (patterns of stereochemistry of chiral linkage phosphorus). Certain useful patterns of backbone chiral centers are described in the present disclosure. In some embodiments, a plurality of oligonucleotides share a common pattern of backbone chiral centers, which is or comprises a pattern described in the present disclosure (e.g., as in “Linkage Phosphorus Stereochemistry and Patterns Thereof”, a pattern of backbone chiral centers of a chirally controlled oligonucleotide in Table 1, etc.).

In some embodiments, a chirally controlled oligonucleotide composition is a chirally pure (or stereopure, stereochemically pure) oligonucleotide composition, wherein the oligonucleotide composition comprises a plurality of oligonucleotides, wherein the oligonucleotides are identical [including that each chiral element of the oligonucleotides, including each chiral linkage phosphorus, is independently defined (stereodefined)], and the composition does not contain other stereoisomers. A chirally pure (or stereopure, stereochemically pure) oligonucleotide composition of an oligonucleotide stereoisomer does not contain other stereoisomers (as appreciated by those skilled in the art, one or more unintended stereoisomers may exist as impurities).

Chirally controlled oligonucleotide compositions can demonstrate a number of advantages over stereorandom oligonucleotide compositions. Among other things, chirally controlled oligonucleotide compositions are more uniform than corresponding stereorandom oligonucleotide compositions with respect to oligonucleotide structures. By controlling stereochemistry, compositions of individual stereoisomers can be prepared and assessed, so that chirally controlled oligonucleotide composition of stereoisomers with desired properties and/or activities can be developed. In some embodiments, chirally controlled oligonucleotide compositions provides better delivery, stability, clearance, activity, selectivity, and/or toxicity profiles compared to, e.g., corresponding stereorandom oligonucleotide compositions. In some embodiments, chirally controlled oligonucleotide compositions provide better efficacy, fewer side effects, and/or more convenient and effective dosage regimens. Among other things, patterns of backbone chiral centers as described herein optionally combined with other structural features described herein, e.g., modifications of nucleobases, sugars, internucleotidic linkages, etc. can be utilized to provide to provide directed adenosine editing with high efficiency.

In some embodiments, an oligonucleotide composition comprises one or more internucleotidic linkages which are stereocontrolled (chirally controlled; in some embodiments, stereopure) and one or more internucleotidic linkages which are stereorandom. In some embodiments, an oligonucleotide composition comprises one or more internucleotidic linkages which are stereocontrolled (chirally controlled; in some embodiments, stereopure) and one or more internucleotidic linkages which are stereorandom.

In some embodiments, an oligonucleotide composition comprises one or more internucleotidic linkages which are stereocontrolled (e.g., chirally controlled or stereopure) and one or more internucleotidic linkages which are stereorandom. Such oligonucleotides may target various nucleic acids and may have various base sequences, and may provide efficient adenosine editing (e.g., conversion of A to I).

In some embodiments, the present disclosure provides a chirally controlled oligonucleotide composition. In some embodiments, provided chirally controlled oligonucleotide compositions comprise a plurality of oligonucleotides of the same constitution, and have one or more internucleotidic linkages. In some embodiments, a plurality of oligonucleotides, e.g., in a chirally controlled oligonucleotide composition, is a plurality of an oligonucleotide selected from Table 1 (and/or one or more of various salts forms thereof), wherein the oligonucleotide comprises at least one Rp or Sp linkage phosphorus in a chirally controlled internucleotidic linkage. In some embodiments, a plurality of oligonucleotides, e.g., in a chirally controlled oligonucleotide composition, is a plurality of an oligonucleotide selected from Table 1 (and/or one or more of various salts forms thereof), wherein each phosphorothioate internucleotidic linkage in the oligonucleotide is independently chirally controlled (each phosphorothioate internucleotidic linkage is independently Rp or Sp). In some embodiments, an oligonucleotide composition, e.g., an oligonucleotide composition is a substantially pure preparation of a single oligonucleotide in that oligonucleotides in the composition that are not the single oligonucleotide are impurities from the preparation process of the single oligonucleotide, in some case, after certain purification procedures. In some embodiments, a single oligonucleotide is an oligonucleotide of Table 1, wherein each chiral internucleotidic linkage of the oligonucleotide is chirally controlled (e.g., indicated as S or R but not X in “Stereochemistry/Linkage”).

In some embodiments, a chirally controlled oligonucleotide composition can have, relative to a corresponding stereorandom oligonucleotide composition, increased activity and/or stability, increased delivery, and/or decreased ability to elicit adverse effects such as complement, TLR9 activation, etc. In some embodiments, a stereorandom (non-chirally controlled) oligonucleotide composition differs from a chirally controlled oligonucleotide composition in that its corresponding plurality of oligonucleotides do not contain any chirally controlled internucleotidic linkages but the stereorandom oligonucleotide composition is otherwise identical to the chirally controlled oligonucleotide composition.

In some embodiments, the present disclosure pertains to a chirally controlled oligonucleotide composition which is capable of modulating level, activity or expression of a gene or a gene product thereof.

In some embodiments, level, activity or expression of a gene or a gene product thereof is increased (e.g., through conversion of A to I to restore correct G to A mutations, to increase protein translation levels, to increase production of particular protein isoforms, to modulate splicing to increase levels of a particular splicing products and proteins encoded thereby, etc.), and in some embodiments, level, activity or expression of a gene or a gene product thereof is decreased (e.g., through conversion of A to I to create stop codon and/or alter codons, to decrease protein translation levels, to decrease production of particular protein isoforms, to modulate splicing to decrease levels of a particular splicing products and proteins encoded thereby, etc.), as compared to a reference condition (e.g., absence of oligonucleotides and/or compositions of the present disclosure, and/or presence of a reference oligonucleotide and/or oligonucleotide composition (e.g., oligonucleotides of the same base sequence but different modifications, stereorandom compositions of oligonucleotides of comparable structures (e.g., base sequence, modifications, etc.) but lack of stereochemical control, etc.).

In some embodiments, the present disclosure provides a chirally controlled oligonucleotide composition which is capable of increasing the level, activity or expression of a gene or a gene product thereof, and comprises a plurality of oligonucleotides which share a common base sequence that is, comprises, or comprises a span (e.g., at least 10 or 15 contiguous bases) of a base sequence disclosed herein (e.g., in Table 1, wherein each T may be independently replaced with U and vice versa). In some embodiments, the present disclosure provides a chirally controlled oligonucleotide composition which is capable of increasing the level, activity or expression of a gene or a gene product thereof, and comprises a plurality of oligonucleotides which share a common base sequence that is or comprises a base sequence disclosed herein (e.g., in Table 1, wherein each T may be independently replaced with U and vice versa). In some embodiments, the present disclosure provides a chirally controlled oligonucleotide composition which is capable of increasing the level, activity or expression of a gene or a gene product thereof, and comprises a plurality of oligonucleotides which share a common base sequence that is a base sequence disclosed herein (e.g., in Table 1, wherein each T may be independently replaced with U and vice versa).

In some embodiments, the present disclosure provides a chirally controlled oligonucleotide composition which is capable of decreasing the level, activity or expression of a gene or a gene product thereof, and comprises a plurality of oligonucleotides which share a common base sequence that is, comprises, or comprises a span (e.g., at least 10 or 15 contiguous bases) of a base sequence disclosed herein (e.g., in Table 1, wherein each T may be independently replaced with U and vice versa). In some embodiments, the present disclosure provides a chirally controlled oligonucleotide composition which is capable of decreasing the level, activity or expression of a gene or a gene product thereof, and comprises a plurality of oligonucleotides which share a common base sequence that is or comprises a base sequence disclosed herein (e.g., in Table 1, wherein each T may be independently replaced with U and vice versa). In some embodiments, the present disclosure provides a chirally controlled oligonucleotide composition which is capable of decreasing the level, activity or expression of a gene or a gene product thereof, and comprises a plurality of oligonucleotides which share a common base sequence that is a base sequence disclosed herein (e.g., in Table 1, wherein each T may be independently replaced with U and vice versa).

In some embodiments, a provided chirally controlled oligonucleotide composition is a chirally controlled oligonucleotide composition comprising a plurality of oligonucleotide. In some embodiments, a chirally controlled oligonucleotide composition is a chirally pure (or “stereochemically pure”) oligonucleotide composition. In some embodiments, the present disclosure provides a chirally pure oligonucleotide composition of an oligonucleotide in Table 1, wherein each chiral internucleotidic linkage of the oligonucleotide is independently chirally controlled (Rp or Sp, e.g., can be determined from R or S but not X in “Stereochemistry/Linkage”). As one of ordinary skill in the art will understand, chemical selectivity rarely, if ever, achieves completeness (absolute 100%). In some embodiments, a chirally pure oligonucleotide composition comprises a plurality of oligonucleotides, wherein oligonucleotides of the plurality are structurally identical and all have the same structure (the same stereoisomeric form; in the context of oligonucleotide, typically the same diastereomeric form as typically multiple chiral centers exist in an oligonucleotide), and the chirally pure oligonucleotide composition does not contain any other stereoisomers (in the context of oligonucleotide, typically diastereomers as typically multiple chiral centers exist in an oligonucleotide; to the extent, e.g., achievable by stereoselective preparation). As appreciated by those skilled in the art, stereorandom (or “racemic”, “non-chirally controlled”) oligonucleotide compositions are random mixtures of many stereoisomers (e.g., 2ⁿ diastereoisomers wherein n is the number of chiral linkage phosphorus for oligonucleotides in which other chiral centers (e.g., carbon chiral centers in sugars) are chirally controlled each independently existing in one configuration and only chiral linkage phosphorus centers are not chirally controlled).

Certain data showing properties and/or activities of chirally controlled oligonucleotide composition, e.g., chirally controlled oligonucleotide composition in modulating level, activity and/or expression of target genes and/or products thereof, are shown in, for example, the Examples of this disclosure.

In some embodiments, the present disclosure provides an oligonucleotide composition comprising oligonucleotides that comprise at least one chiral linkage phosphorus. In some embodiments, the present disclosure provides an oligonucleotide composition comprising oligonucleotides that comprise at least one chiral linkage phosphorus. In some embodiments, the present disclosure provides an oligonucleotide composition in which the oligonucleotides comprise a chirally controlled phosphorothioate internucleotidic linkage, wherein the linkage phosphorus has a Rp configuration. In some embodiments, the present disclosure provides an oligonucleotide composition in which the oligonucleotides comprise a chirally controlled phosphorothioate internucleotidic linkage, wherein the linkage phosphorus has a Sp configuration. In some embodiments, the present disclosure provides an oligonucleotide composition in which the oligonucleotides comprise a chirally controlled phosphorothioate internucleotidic linkage, wherein the linkage phosphorus has a Rp configuration and the linkage phosphorus has a Sp configuration. In some embodiments, such oligonucleotide compositions are chirally controlled, and the Rp and/or Sp internucleotidic linkages are independently chirally controlled internucleotidic linkages.

In some embodiments, compared to reference oligonucleotides or oligonucleotide compositions, provided oligonucleotides or oligonucleotide compositions (e.g., chirally controlled oligonucleotide compositions) are surprisingly effective. In some embodiments, desired biological effects (e.g., as measured by increased (if increase is desired) and/or decreased (if decrease is desired) levels of mRNA, proteins, etc. whose levels are targeted for increase) can be enhanced by more than 1.5, 2, 3, 4, 5, 6, 7, 8, 9, 10, 15, 20, 25, 30, 40, 50, or 100 fold (e.g., as measured by levels of desired mRNA, proteins, etc.). In some embodiments, a change is measured by increase of desired mRNA and/or protein levels, or decrease of undesired mRNA and/or protein levels, compared to a reference condition. In some embodiments, a change is measured by increase of a desired mRNA and/or protein level compared to a reference condition. In some embodiments, a change is measured by decrease of an undesired mRNA and/or level compared to a reference condition. In some embodiments, a reference condition is absence of provided oligonucleotides or oligonucleotide compositions, and or presence of reference oligonucleotides or oligonucleotide compositions, respectively. In some embodiments, a reference oligonucleotide shares the same base sequence, but different nucleobase modifications, sugar modifications, internucleotidic linkages modifications, and/or linkage phosphorus stereochemistry. In some embodiments, a reference oligonucleotide composition is a composition of oligonucleotides of the same base sequence, but different nucleobase modifications, sugar modifications, internucleotidic linkages modifications, and/or linkage phosphorus stereochemistry. In some embodiments, a reference composition for a chirally controlled oligonucleotide composition is a corresponding stereorandom composition of oligonucleotides having the same base sequence, nucleobase modifications, sugar modifications, and/or internucleotidic linkages modifications (but lack of and/or low levels of linkage phosphorus stereochemistry control), or having the same constitution.

In some embodiments, the present disclosure provides a chirally controlled oligonucleotide composition, wherein the linkage phosphorus of at least one chirally controlled internucleotidic linkage is Sp. In some embodiments, the present disclosure provides a chirally controlled oligonucleotide composition, wherein the majority of linkage phosphorus of chirally controlled internucleotidic linkages are Sp. In some embodiments, about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more, of all chirally controlled internucleotidic linkages (or of all chiral internucleotidic linkages, or of all internucleotidic linkages) are Sp. In some embodiments, about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more, of all chirally controlled phosphorothioate internucleotidic linkages are Sp. In some embodiments, no more than 1, 2, 3, 4, 5, 6, 7, 8, 9, or 10 of phosphorothioate internucleotidic linkages are non-chirally controlled or are chirally controlled and Rp. In some embodiments, no more than 1, 2, 3, 4, 5, 6, 7, 8, 9, or 10 of phosphorothioate internucleotidic linkages are are chirally controlled and Rp. In some embodiments, it is no more than 1. In some embodiments, it is no more than 2. In some embodiments, it is no more than 3. In some embodiments, it is no more than 4. In some embodiments, it is no more than 5. In some embodiments, each phosphorothioate internucleotidic linkage is independently chirally controlled. In some embodiments, the present disclosure provides a chirally controlled oligonucleotide composition, wherein the majority of chiral internucleotidic linkages are chirally controlled and are Sp at their linkage phosphorus. In some embodiments, the present disclosure provides a chirally controlled oligonucleotide composition, wherein each chiral internucleotidic linkage is chirally controlled and each chiral linkage phosphorus is Sp. In some embodiments, the present disclosure provides a chirally controlled oligonucleotide composition, e.g., chirally controlled oligonucleotide composition, wherein at least one chirally controlled internucleotidic linkage has a Rp linkage phosphorus. In some embodiments, the present disclosure provides a chirally controlled oligonucleotide composition, wherein at least one chirally controlled internucleotidic linkage comprises a Rp linkage phosphorus and at least one chirally controlled internucleotidic linkage comprises a Sp linkage phosphorus.

In some embodiments, the present disclosure provides a chirally controlled oligonucleotide composition, wherein at least two chirally controlled internucleotidic linkages have different linkage phosphorus stereochemistry and/or different P-modifications relative to one another, wherein a P-modification is a modification at a linkage phosphorus. In some embodiments, the present disclosure provides a chirally controlled oligonucleotide composition, wherein at least two chirally controlled internucleotidic linkages have different stereochemistry relative to one another, and the pattern of the backbone chiral centers of the oligonucleotides is characterized by a repeating pattern of alternating stereochemistry.

In certain embodiments, the present disclosure provides a chirally controlled oligonucleotide composition comprising a plurality of oligonucleotides, wherein with in each of the oligonucleotides at least two individual internucleotidic linkages have different P-modifications relative to one another. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage and a phosphorothioate internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate triester internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage and a phosphorothioate triester internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate internucleotidic linkage and a phosphorothioate triester internucleotidic linkage.

In some embodiments, the present disclosure provides a chirally controlled oligonucleotide composition, comprising a plurality of oligonucleotides which share a common base sequence that is the base sequence of an oligonucleotide disclosed herein, wherein at least one internucleotidic linkage is chirally controlled.

### Linkage Phosphorus Stereochemistry and Pattern of Backbone Chiral Centers

Among other things, the present disclosure provides various oligonucleotide compositions. In some embodiments, the present disclosure provides oligonucleotide compositions of oligonucleotides described herein. In some embodiments, an oligonucleotide composition comprises a plurality of oligonucleotides described in the present disclosure. In some embodiments, an oligonucleotide composition is chirally controlled. In some embodiments, an oligonucleotide composition is not chirally controlled (stereorandom).

In contrast to natural phosphate linkages, linkage phosphorus of chiral modified internucleotidic linkages, e.g., phosphorothioate internucleotidic linkages, are chiral. Among other things, the present disclosure provides technologies (e.g., oligonucleotides, compositions, methods, etc.) comprising control of stereochemistry of chiral linkage phosphorus in chiral internucleotidic linkages. In some embodiments, as demonstrated herein, control of stereochemistry can provide improved properties and/or activities, including desired stability, reduced toxicity, improved modification of target nucleic acids, improved modulation of levels of transcripts and/or products (e.g., mRNA, proteins, etc.) encoded thereof, etc. In some embodiments, the present disclosure provides useful patterns of backbone chiral centers for oligonucleotides and/or regions thereof, which pattern includes a combination of stereochemistry of each chiral linkage phosphorus (Rp or Sp) of chiral linkage phosphorus, indication of each achiral linkage phosphorus (Op, if any), etc. from 5′ to 3′. Certain patterns are provided in various Tables (e.g., Stereochemistry/Linkage as examples; such patterns can be applied to various oligonucleotides with various base sequences and modifications (e.g., those described herein including patterns thereof).

Useful patterns of backbone chiral centers, e.g., those for oligonucleotides, first domains, second domains, first subdomains, second subdomains, third subdomains, etc., are extensively described herein. For example, in some embodiments, high levels of Sp internucleotidic linkages of oligonucleotides or of one or more portions thereof (e.g., first domains, second domains, first subdomains, second subdomains, and/or third subdomains, and/or 5′-end portions and/or 3′-end portions therein) provide high stability and/or activities. In some embodiments, first domains contain high levels of Sp internucleotidic linkages. In some embodiments, second domains contain high levels of Sp internucleotidic linkages (in numbers and/or percentages, relative to natural phosphate linkages and/or Rp internucleotidic linkages). In some embodiments, first subdomains contain high levels of Sp internucleotidic linkages. In some embodiments, second subdomains contain high levels of Sp internucleotidic linkages. In some embodiments, third subdomains contain high levels of Sp internucleotidic linkages. In some embodiments, as demonstrated herein Rp internucleotidic linkages can be utilized in various locations and/or portions. For example, in some embodiments, first domains contain one or more or high levels of Rp internucleotidic linkages, and in some embodiments, second subdomains contain one or more or high levels of Rp internucleotidic linkages.

In some embodiments, a number of linkage phosphorus in chirally controlled internucleotidic linkages are Sp. In some embodiments, at least 10%, 20%, 25%, 30%, 35%, 40%, 45%, 50%, 55%, 60%, 65%, 70%, 75%, 80%, 85%, 90% or 95% of chirally controlled internucleotidic linkages have Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all chiral internucleotidic linkages are chirally controlled internucleotidic linkages having Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all internucleotidic linkages are chirally controlled internucleotidic linkages having Sp linkage phosphorus. In some embodiments, the percentage is at least 20%. In some embodiments, the percentage is at least 30%. In some embodiments, the percentage is at least 40%. In some embodiments, the percentage is at least 50%. In some embodiments, the percentage is at least 60%. In some embodiments, the percentage is at least 65%. In some embodiments, the percentage is at least 70%. In some embodiments, the percentage is at least 75%. In some embodiments, the percentage is at least 80%. In some embodiments, the percentage is at least 90%. In some embodiments, the percentage is at least 95%. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Sp linkage phosphorus. In some embodiments, at least 5 internucleotidic linkages are chirally controlled internucleotidic linkages having Sp linkage phosphorus. In some embodiments, at least 6 internucleotidic linkages are chirally controlled internucleotidic linkages having Sp linkage phosphorus. In some embodiments, at least 7 internucleotidic linkages are chirally controlled internucleotidic linkages having Sp linkage phosphorus. In some embodiments, at least 8 internucleotidic linkages are chirally controlled internucleotidic linkages having Sp linkage phosphorus. In some embodiments, at least 9 internucleotidic linkages are chirally controlled internucleotidic linkages having Sp linkage phosphorus. In some embodiments, at least 10 internucleotidic linkages are chirally controlled internucleotidic linkages having Sp linkage phosphorus. In some embodiments, at least 11 internucleotidic linkages are chirally controlled internucleotidic linkages having Sp linkage phosphorus. In some embodiments, at least 12 internucleotidic linkages are chirally controlled internucleotidic linkages having Sp linkage phosphorus. In some embodiments, at least 13 internucleotidic linkages are chirally controlled internucleotidic linkages having Sp linkage phosphorus. In some embodiments, at least 14 internucleotidic linkages are chirally controlled internucleotidic linkages having Sp linkage phosphorus. In some embodiments, at least 15 internucleotidic linkages are chirally controlled internucleotidic linkages having Sp linkage phosphorus. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In some embodiments, no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 internucleotidic linkages are chirally controlled internucleotidic linkages having Rp linkage phosphorus. In some embodiments, one and no more than one internucleotidic linkage in an oligonucleotide is a chirally controlled internucleotidic linkage having Rp linkage phosphorus. In some embodiments, 2 and no more than 2 internucleotidic linkages in an oligonucleotide are chirally controlled internucleotidic linkages having Rp linkage phosphorus. In some embodiments, 3 and no more than 3 internucleotidic linkages in an oligonucleotide are chirally controlled internucleotidic linkages having Rp linkage phosphorus. In some embodiments, 4 and no more than 4 internucleotidic linkages in an oligonucleotide are chirally controlled internucleotidic linkages having Rp linkage phosphorus. In some embodiments, 5 and no more than 5 internucleotidic linkages in an oligonucleotide are chirally controlled internucleotidic linkages having Rp linkage phosphorus.

In some embodiments, all, essentially all or most of the internucleotidic linkages in an oligonucleotide or a portion thereof are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages in an oligonucleotide) except for one or a minority of internucleotidic linkages (e.g., 1, 2, 3, 4, or 5, and/or less than 50%, 45%, 40%, 35%, 30%, 25%, 20%, 15%, 10%, or 5% of all chirally controlled internucleotidic linkages, or of all chiral internucleotidic linkages, or of all internucleotidic linkages in an oligonucleotide) being in the Rp configuration. In some embodiments, all, essentially all or most of the internucleotidic linkages in a first domain are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in a first domain). In some embodiments, each internucleotidic linkage in a first domain is a phosphorothioate in the Sp configuration. In some embodiments, each internucleotidic linkage in the a domain is a phosphorothioate in the Sp configuration. In some embodiments, all, essentially all or most of the internucleotidic linkages in a second domain are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in a second domain). In some embodiments, each internucleotidic linkage in a second domain is a phosphorothioate in the Sp configuration. In some embodiments, each internucleotidic linkage in a second domain is a phosphorothioate in the Sp configuration except for one phosphorothioate in the Rp configuration. In some embodiments, all, essentially all or most of the internucleotidic linkages in a subdomain of a second domain are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in a first subdomain of a second domain). In some embodiments, each internucleotidic linkage in a first subdomain of a second domain is a phosphorothioate in the Sp configuration. In some embodiments, each internucleotidic linkage in a first subdomain of second domain is a phosphorothioate in the Sp configuration except for one phosphorothioate in the Rp configuration. In some embodiments, all, essentially all or most of the internucleotidic linkages in a the second subdomain of a second domain are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in a second subdomain of a second domain) except for one or a minority of internucleotidic linkages being in the Rp configuration. In some embodiments, each internucleotidic linkage in a second subdomain of a second domain is a phosphorothioate in the Sp configuration except for one phosphorothioate in the Rp configuration. In some embodiments, each internucleotidic linkage in a second subdomain of a second domain is a phosphorothioate in the Sp configuration except for one phosphorothioate in the Rp configuration. In some embodiments, all, essentially all or most of the internucleotidic linkages in a the third subdomain of the second domain are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in a third subdomain of a second domain. In some embodiments, each internucleotidic linkage in a third subdomain of a second domain is a phosphorothioate in the Sp configuration except for one phosphorothioate in the Rp configuration. In some embodiments, each internucleotidic linkage in a third subdomain of a second domain is a phosphorothioate in the Sp configuration except for one phosphorothioate in the Rp configuration.

In some embodiments, an oligonucleotide comprises one or more Rp internucleotidic linkages. In some embodiments, an oligonucleotide comprises one and no more than one Rp internucleotidic linkages. In some embodiments, an oligonucleotide comprises five or more Rp internucleotidic linkages. In some embodiments, about 5%-50% of all chirally controlled internucleotidic linkages in an oligonucleotide are Rp. In some embodiments, about 5%-40% of all chirally controlled internucleotidic linkages in an oligonucleotide are Rp. In some embodiments, certain portions (e.g., domains, subdomains, etc.) may contain relatively more (in numbers and/or percentages) Rp internucleotidic linkages, e.g., second subdomains.

In some embodiments, the present disclosure provides a chirally controlled oligonucleotide composition wherein the composition comprises a non-random or controlled level of a plurality of oligonucleotides, wherein oligonucleotides of the plurality share a common base sequence, and share the same configuration of linkage phosphorus independently at 1-60, 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more chiral internucleotidic linkages.

In some embodiments, provided oligonucleotides comprise 2-30 chirally controlled internucleotidic linkages. In some embodiments, provided oligonucleotide compositions comprise 5-30 chirally controlled internucleotidic linkages. In some embodiments, provided oligonucleotide compositions comprise 10-30 chirally controlled internucleotidic linkages. In some embodiments, provided oligonucleotide compositions comprise 1, 2, 3, 4, 5, 6, 7, 8, 9, 10, 11, 12, 13, 14, 15, 16, 17, 18, 19, 20, 21, 22, 23, 24, or 25 or more chirally controlled internucleotidic linkages.

In some embodiments, about 1-100% of all internucleotidic linkages are chirally controlled internucleotidic linkages. In some embodiments, about 1-100% of all chiral internucleotidic linkages are chirally controlled internucleotidic linkages. In some embodiments, a percentage is about 5%-100%. In some embodiments, a percentage is at least 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%. In some embodiments, a percentage is about 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%.

In some embodiments, an internucleotidic linkage in the Sp configuration (having a Sp linkage phosphorus) is a phosphorothioate internucleotidic linkage. In some embodiments, an achiral internucleotidic linkage is a natural phosphate linkage. In some embodiments, an internucleotidic linkage in the Rp configuration (having a Rp linkage phosphorus) is a phosphorothioate internucleotidic linkage. In some embodiments, each internucleotidic linkage in the Sp configuration is a phosphorothioate internucleotidic linkage. In some embodiments, each achiral internucleotidic linkage is a natural phosphate linkage. In some embodiments, each internucleotidic linkage in the Rp configuration is a phosphorothioate internucleotidic linkage. In some embodiments, each internucleotidic linkage in the Sp configuration is a phosphorothioate internucleotidic linkage, each achiral internucleotidic linkage is a natural phosphate linkage, and each internucleotidic linkage in the Rp configuration is a phosphorothioate internucleotidic linkage.

In some embodiments, provided oligonucleotides in chirally controlled oligonucleotide compositions each comprise different types of internucleotidic linkages. In some embodiments, provided oligonucleotides comprise at least one natural phosphate linkage and at least one modified internucleotidic linkage. In some embodiments, provided oligonucleotides comprise at least one natural phosphate linkage and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 modified internucleotidic linkages. In some embodiments, a modified internucleotidic linkage is a phosphorothioate internucleotidic linkage. In some embodiments, each modified internucleotidic linkage is a phosphorothioate internucleotidic linkage. In some embodiments, each modified internucleotidic linkage is independently a chiral internucleotidic linkage and is independently chirally controlled.

In some embodiments, oligonucleotides in a chirally controlled oligonucleotide composition each comprise at least two internucleotidic linkages that have different stereochemistry and/or different P-modifications relative to one another. In some embodiments, at least two internucleotidic linkages have different stereochemistry relative to one another. In some embodiments, oligonucleotides each comprise a pattern of backbone chiral centers comprising alternating linkage phosphorus stereochemistry.

In some embodiments, a phosphorothioate triester linkage comprises a chiral auxiliary, which, for example, is used to control the stereoselectivity of a reaction, e.g., a coupling reaction in an oligonucleotide synthesis cycle. In some embodiments, a phosphorothioate triester linkage does not comprise a chiral auxiliary. In some embodiments, a phosphorothioate triester linkage is intentionally maintained until and/or during the administration of the oligonucleotide composition to a subject.

In some embodiments, oligonucleotides are linked to a solid support. In some embodiments, a solid support is a support for oligonucleotide synthesis. In some embodiments, a solid support comprises glass. In some embodiments, a solid support is CPG (controlled pore glass). In some embodiments, a solid support is polymer. In some embodiments, a solid support is polystyrene. In some embodiments, the solid support is Highly Crosslinked Polystyrene (HCP). In some embodiments, the solid support is hybrid support of Controlled Pore Glass (CPG) and Highly Cross-linked Polystyrene (HCP). In some embodiments, a solid support is a metal form. In some embodiments, a solid support is a resin. In some embodiments, oligonucleotides are cleaved from a solid support.

In some embodiments, purity, particularly stereochemical purity, and particularly diastereomeric purity of many oligonucleotides and compositions thereof wherein all other chiral centers in the oligonucleotides but the chiral linkage phosphorus centers have been stereodefined (e.g., carbon chiral centers in the sugars, which are defined in, e.g., phosphoramidites for oligonucleotide synthesis), can be controlled by stereoselectivity (as appreciated by those skilled in this art, diastereoselectivity in many cases of oligonucleotide synthesis wherein the oligonucleotide comprise more than one chiral centers) at chiral linkage phosphorus in coupling steps when forming chiral internucleotidic linkages. In some embodiments, a coupling step has a stereoselectivity (diastereoselectivity when there are other chiral centers) of 60% at the linkage phosphorus. After such a coupling step, the new internucleotidic linkage formed may be referred to have a 60% stereochemical purity (for oligonucleotides, typically diastereomeric purity in view of the existence of other chiral centers). In some embodiments, each coupling step independently has a stereoselectivity of at least 60%, 70%, 80%, 85%, 90%, 91%, 92%, 93%, 94%, 95%, 96%, 97%, 98%, 99%, or 99.5%. In some embodiments, a chirally controlled internucleotidic linkage is typically formed with a stereoselectivity of at least 85%, 87%, 90%, 91%, 92%, 93%, 94%, 95%, 96%, 97%, 98%, 99.5% or virtually 100% (in some embodiments, at least 85%; in some embodiments, at least 87%; in some embodiments, at least 90%; in some embodiments, at least 95%; in some embodiments, at least 96%; in some embodiments, at least 97%; in some embodiments, at least 98%; in some embodiments, at least 99%). In some embodiments, a stereoselectivity is at least 85%. In some embodiments, a stereoselectivity is at least 87%. In some embodiments, a stereoselectivity is at least 90%. In some embodiments, each coupling step independently has a stereoselectivity of virtually 100%.

In some embodiments, stereopurity of a chiral center, e.g., a chiral linkage phosphorus, in a composition is at least 60%, 70%, 80%, 85%, 87%, 90%, 91%, 92%, 93%, 94%, 95%, 96%, 97%, 98%, 99%, or 99.5%. In some embodiments, a stereopurity is at least 80%. In some embodiments, a stereopurity is at least 85%. In some embodiments, a stereopurity is at least 87%. In some embodiments, a stereopurity is at least 90%. In some embodiments, a stereopurity is virtually 100%. In some embodiments, each chirally controlled internucleotidic linkage independently has a stereochemical purity (typically diastereomeric purity for oligonucleotides with multiple chiral centers) of at least 85%, 87%, 90%, 91%, 92%, 93%, 94%, 95%, 96%, 97%, 98%, 99.5% or virtually 100% (in some embodiments, at least 85%; in some embodiments, at least 87%; in some embodiments, at least 90%; in some embodiments, at least 95%; in some embodiments, at least 96%; in some embodiments, at least 97%; in some embodiments, at least 98%; in some embodiments, at least 99%) at its chiral linkage phosphorus. In some embodiments, a chirally controlled internucleotidic linkage has a stereochemical purity of at least 90%. In some embodiments, a majority of chirally controlled internucleotidic linkages independently have a stereochemical purity of at least 90%. In some embodiments, each chirally controlled internucleotidic linkage independently has a stereochemical purity of at least 90%. In some embodiments, each phosphorothioate internucleotidic linkage is independently chirally controlled. In some embodiments, at least 60%, 65%, 70%, 75%, 80%, 85%, 90%, or 95%, or all chirally controlled internucleotidic linkages are Sp. In some embodiments, at least 60%, 65%, 70%, 75%, 80%, 85%, 90%, or 95%, or all chirally controlled phosphorothioate internucleotidic linkages are Sp. In some embodiments, at least 60%, 65%, 70%, 75%, 80%, 85%, 90%, or 95%, or all phosphorothioate internucleotidic linkages are chirally controlled and are Sp.

Stereoselectivity and stereopurity may be assessed by various technologies. In some embodiments, stereoselectivity and/or stereopurity is virtually 100% in that when a composition is analyzed by an analytical method (e.g., NMR, HPLC, etc.), virtually all detectable stereoisomers has the intended stereochemistry.

In some embodiments, at least 1, 2, 3, 4, 5, 6, 7, 8, 9, or 10 couplings of a monomer (as appreciated by those skilled in the art in many embodiments a phosphoramidite for oligonucleotide synthesis) independently have a stereoselectivity less than about 60%, 70%, 80%, 85%, or 90% [for oligonucleotide synthesis, typically diastereoselectivity with respect to formed linkage phosphorus chiral center(s)].

In some embodiments, in stereorandom (or racemic) preparations (or stereorandom/non-chirally controlled oligonucleotide compositions), at least 1, 2, 3, 4, 5, 6, 7, 8, 9, 10, 11, 12, 13, 14, 15, 16, 17, 18, 19, 20, 21, 22, 23, 24, or 25 chiral internucleotidic linkages of the oligonucleotides independently have a stereochemical purity (typically diastereomeric purity for oligonucleotides comprising multiple chiral centers) less than about 60%, 65%, 70%, 75%, 80%, or 85% with respect to chiral linkage phosphorus of the internucleotidic linkage(s). In some embodiments, a stereochemistry purity (stereopurity) is less than about 60%. In some embodiments, a stereochemistry purity (stereopurity) is less than about 65%. In some embodiments, a stereochemistry purity (stereopurity) is less than about 70%. In some embodiments, a stereochemistry purity (stereopurity) is less than about 75%. In some embodiments, a stereochemistry purity (stereopurity) is less than about 80%.

In some embodiments, compounds of the present disclosure (e.g., oligonucleotides, chiral auxiliaries, etc.) comprise multiple chiral elements (e.g., multiple carbon and/or phosphorus (e.g., linkage phosphorus of chiral internucleotidic linkages) chiral centers). In some embodiments, at least 1, 2, 3, 4, 5, 6, 7, 8, 9 or more chiral elements of a provided compound (e.g., an oligonucleotide) each independently have a diastereomeric purity as described herein. In some embodiments, a diastereomeric purity is at least 85%. In some embodiments, a diastereomeric purity is at least 86%. In some embodiments, a diastereomeric purity is at least 87%. In some embodiments, a diastereomeric purity is at least 88%. In some embodiments, a diastereomeric purity is at least 89%. In some embodiments, a diastereomeric purity is at least 90%. In some embodiments, a diastereomeric purity is at least 91%. In some embodiments, a diastereomeric purity is at least 92%. In some embodiments, a diastereomeric purity is at least 93%. In some embodiments, a diastereomeric purity is at least 94%. In some embodiments, a diastereomeric purity is at least 95%. In some embodiments, a diastereomeric purity is at least 96%. In some embodiments, a diastereomeric purity is at least 97%. In some embodiments, a diastereomeric purity is at least 98%. In some embodiments, a diastereomeric purity is at least 99%.

As understood by a person having ordinary skill in the art, in some embodiments, diastereoselectivity of a coupling or diastereomeric purity of a chiral linkage phosphorus center can be assessed through the diastereoselectivity of a dimer formation or diastereomeric purity of a dimer prepared under the same or comparable conditions, wherein the dimer has the same 5′- and 3′-nucleosides and internucleotidic linkage.

Various technologies can be utilized for identifying or confirming stereochemistry of chiral elements (e.g., configuration of chiral linkage phosphorus) and/or patterns of backbone chiral centers, and/or for assessing stereoselectivity (e.g., diastereoselectivity of couple steps in oligonucleotide synthesis) and/or stereochemical purity (e.g., diastereomeric purity of internucleotidic linkages, compounds (e.g., oligonucleotides), etc.). Example technologies include NMR [e.g., 1D (one-dimensional) and/or 2D (two-dimensional)¹H-³¹P HETCOR (heteronuclear correlation spectroscopy)], HPLC, RP-HPLC, mass spectrometry, LC-MS, and cleavage of internucleotidic linkages by stereospecific nucleases, etc., which may be utilized individually or in combination. Example useful nucleases include benzonase, micrococcal nuclease, and svPDE (snake venom phosphodiesterase), which are specific for certain internucleotidic linkages with Rp linkage phosphorus (e.g., a Rp phosphorothioate linkage); and nuclease P1, mung bean nuclease, and nuclease 51, which are specific for internucleotidic linkages with Sp linkage phosphorus (e.g., a Sp phosphorothioate linkage). Without wishing to be bound by any particular theory, the present disclosure notes that, in at least some cases, cleavage of oligonucleotides by a particular nuclease may be impacted by structural elements, e.g., chemical modifications (e.g., 2′-modifications of a sugars), base sequences, or stereochemical contexts. For example, it is observed that in some cases, benzonase and micrococcal nuclease, which are specific for internucleotidic linkages with Rp linkage phosphorus, were unable to cleave an isolated Rp phosphorothioate internucleotidic linkage flanked by Sp phosphorothioate internucleotidic linkages.

In some embodiments, oligonucleotides sharing a common base sequence, a common pattern of backbone linkages, and a common pattern of backbone chiral centers share a common pattern of backbone phosphorus modifications and a common pattern of base modifications. In some embodiments, oligonucleotide compositions sharing a common base sequence, a common pattern of backbone linkages, and a common pattern of backbone chiral centers share a common pattern of backbone phosphorus modifications and a common pattern of nucleoside modifications. In some embodiments, oligonucleotides share a common base sequence, a common pattern of backbone linkages, and a common pattern of backbone chiral centers have identical structures.

In some embodiments, the present disclosure provides an oligonucleotide composition comprising a plurality of oligonucleotides capable of directing deamination of a target adenosine in a target nucleic acid, wherein oligonucleotides of the plurality are of a particular oligonucleotide type, which composition is chirally controlled in that it is enriched, relative to a substantially racemic preparation of oligonucleotides having the same base sequence, for oligonucleotides of the particular oligonucleotide type.

In some embodiments, a plurality of oligonucleotides or oligonucleotides of a particular oligonucleotide type in a provided oligonucleotide composition are oligonucleotides. In some embodiments, the present disclosure provides a chirally controlled oligonucleotide composition comprising a plurality of oligonucleotides, wherein the oligonucleotides share:

a common base sequence;

a common pattern of backbone linkages; and

the same linkage phosphorus stereochemistry at one or more chiral internucleotidic linkages (chirally controlled internucleotidic linkages),

wherein the composition is enriched, relative to a substantially racemic preparation of oligonucleotides sharing the common base sequence and pattern of backbone linkages, for oligonucleotides of the plurality.

In some embodiments, the present disclosure provides a chirally controlled oligonucleotide composition comprising a plurality of oligonucleotides, wherein the oligonucleotides share:

a common base sequence;

a common pattern of backbone linkages; and

a common pattern of backbone chiral centers, which composition is a substantially pure preparation of a single oligonucleotide in that at least about 10%, 15%, 20%, 25%, 30%, 35%, 40%, 45%, 50%, 55%, 60%, 65%, 70%, 75%, 80%, 85%, 90%, 91%, 92%, 93%, 93%, 95%, 96%, 97%, 98%, or 99% of the oligonucleotides in the composition have the common base sequence, the common pattern of backbone linkages, and the common pattern of backbone chiral centers.

In some embodiments, an oligonucleotide composition type is further defined by: 4) additional chemical moiety, if any.

In some embodiments, the percentage is at least about 10%. In some embodiments, the percentage is at least about 20%. In some embodiments, the percentage is at least about 30%. In some embodiments, the percentage is at least about 40%. In some embodiments, the percentage is at least about 50%. In some embodiments, the percentage is at least about 60%. In some embodiments, the percentage is at least about 70%. In some embodiments, the percentage is at least about 75%. In some embodiments, the percentage is at least about 80%. In some embodiments, the percentage is at least about 85%. In some embodiments, the percentage is at least about 90%. In some embodiments, the percentage is at least about 91%. In some embodiments, the percentage is at least about 92%. In some embodiments, the percentage is at least about 93%. In some embodiments, the percentage is at least about 94%. In some embodiments, the percentage is at least about 95%. In some embodiments, the percentage is at least about 96%. In some embodiments, the percentage is at least about 97%. In some embodiments, the percentage is at least about 98%. In some embodiments, the percentage is at least about 99%. In some embodiments, the percentage is or is greater than (DS)^{nc}, wherein DS and nc are each independently as described in the present disclosure.

In some embodiments, a plurality of oligonucleotides share the same constitution. In some embodiments, a plurality of oligonucleotides are identical (the same stereoisomer). In some embodiments, a chirally controlled oligonucleotide composition is a stereopure oligonucleotide composition wherein oligonucleotides of the plurality are identical (the same stereoisomer), and the composition does not contain any other stereoisomers. Those skilled in the art will appreciate that one or more other stereoisomers may exist as impurities as processes, selectivities, purifications, etc. may not achieve completeness.

In some embodiments, a provided composition is characterized in that when it is contacted with a target nucleic acid [e.g., a transcript (e.g., pre-mRNA, mature mRNA, other types of RNA, etc. that hybridizes with oligonucleotides of the composition)], levels of the target nucleic acid and/or a product encoded thereby is reduced compared to that observed under a reference condition. In some embodiments, levels of a nucleic acid and/or a product thereof, which nucleic acid is a product of an A to I edition of a target nucleic acid, is increased. In some embodiments, a reference condition is selected from the group consisting of absence of the composition, presence of a reference composition, and combinations thereof. In some embodiments, a reference condition is absence of the composition. In some embodiments, a reference condition is presence of a reference composition. In some embodiments, a reference composition is a composition whose oligonucleotides do not hybridize with the target nucleic acid. In some embodiments, a reference composition is a composition whose oligonucleotides do not comprise a sequence that is sufficiently complementary to the target nucleic acid. In some embodiments, a reference composition is a composition whose oligonucleotides share the same base sequence but do not share the same nucleobase, sugar and/or internucleotidic linkage modifications. In some embodiments, a provided composition is a chirally controlled oligonucleotide composition and a reference composition is a non-chirally controlled oligonucleotide composition which is otherwise identical but is not chirally controlled (e.g., a racemic preparation of oligonucleotides of the same constitution as oligonucleotides of a plurality in the chirally controlled oligonucleotide composition).

In some embodiments, the present disclosure provides a chirally controlled oligonucleotide composition comprising a plurality of oligonucleotides capable of directing deamination of a target adenosine in a target nucleic acid, wherein the oligonucleotides share:

a common base sequence,

a common pattern of backbone linkages, and

the same linkage phosphorus stereochemistry at one or more chiral internucleotidic linkages (chirally controlled internucleotidic linkages),

wherein the composition is enriched, relative to a substantially racemic preparation of oligonucleotides sharing the common base sequence and pattern of backbone linkages, for oligonucleotides of the plurality,

the oligonucleotide composition being characterized in that, when it is contacted with a target sequence, deamination of the target adenosine in the target nucleic acid is improved relative to that observed under a reference condition selected from the group consisting of absence of the composition, presence of a reference composition, and combinations thereof.

As appreciated by those skilled in the art, deamination of a target adenosine can be assessed using various technologies. In some embodiments, a technology is sequencing, wherein a deaminated adenosine is detected as G or I. In some embodiments, deamination is assessed by levels of a product (e.g., RNA, protein (e.g., encoded by a sequence wherein a target A is replaced with I but is otherwise identical to a target nucleic acid), etc.).

As demonstrated herein, oligonucleotide structural elements (e.g., sugar modifications, backbone linkages, backbone chiral centers, backbone phosphorus modifications, patterns thereof, etc.) and combinations thereof can provide surprisingly improved properties and/or bioactivities.

In some embodiments, an oligonucleotide composition is a substantially pure preparation of a single oligonucleotide stereoisomer in that oligonucleotides in the composition that are of the same constitution but are not of the stereoisomer are impurities from the preparation process of said oligonucleotide stereoisomer, in some case, after certain purification procedures.

In some embodiments, the present disclosure provides oligonucleotides and oligonucleotide compositions that are chirally controlled, and in some embodiments, stereopure. For instance, in some embodiments, a provided composition contains non-random or controlled levels of one or more individual oligonucleotide types. In some embodiments, oligonucleotides of the same oligonucleotide type are identical.

### Nucleobases

Various nucleobases may be utilized in provided oligonucleotides in accordance with the present disclosure. In some embodiments, a nucleobase is a natural nucleobase, the most commonly occurring ones being A, T, C, G and U. In some embodiments, a nucleobase is a modified nucleobase in that it is not A, T, C, G or U. In some embodiments, a nucleobase is optionally substituted A, T, C, G or U, or a substituted tautomer of A T, C, G or U. In some embodiments, a nucleobase is optionally substituted A, T, C, G or U, e.g., 5mC, 5-hydroxymethyl C, etc. In some embodiments, a nucleobase is alkyl-substituted A, T, C, G or U. In some embodiments, a nucleobase is A. In some embodiments, a nucleobase is T. In some embodiments, a nucleobase is C. In some embodiments, a nucleobase is G. In some embodiments, a nucleobase is U. In some embodiments, a nucleobase is 5mC. In some embodiments, a nucleobase is substituted A, T, C, G or U. In some embodiments, a nucleobase is a substituted tautomer of A, T, C, G or U. In some embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In some embodiments, modified nucleobases improves properties and/or activities of oligonucleotides. For example, in many cases, 5mC may be utilized in place of C to modulate certain undesired biological effects, e.g., immune responses. In some embodiments, when determining sequence identity, a substituted nucleobase having the same hydrogen-bonding pattern is treated as the same as the unsubstituted nucleobase, e.g., 5mC may be treated the same as C [e.g., an oligonucleotide having 5mC in place of C (e.g., AT5mCG) is considered to have the same base sequence as an oligonucleotide having C at the corresponding location(s) (e.g., ATCG)]. In some embodiments, a nucleobase is or comprise an optionally substituted ring having at least one nitrogen atom. In some embodiments, a nucleobase comprise Ring BA as described herein, wherein at least one monocyclic ring of Ring BA comprise a nitrogen ring atom.

In some embodiments, an oligonucleotide comprises one or more A, T, C, G or U. In some embodiments, an oligonucleotide comprises one or more optionally substituted A, T, C, G or U. In some embodiments, an oligonucleotide comprises one or more 5-methylcytidine, 5-hydroxymethylcytidine, 5-formylcytosine, or 5-carboxylcytosine. In some embodiments, an oligonucleotide comprises one or more 5-methylcytidine. In some embodiments, each nucleobase in an oligonucleotide is selected from the group consisting of optionally substituted A, T, C, G and U, and optionally substituted tautomers of A, T, C, G and U. In some embodiments, each nucleobase in an oligonucleotide is optionally protected A, T, C, G and U. In some embodiments, each nucleobase in an oligonucleotide is optionally substituted A, T, C, G or U. In some embodiments, each nucleobase in an oligonucleotide is selected from the group consisting of A, T, C, G, U, and 5mC.

As demonstrated herein, utilization of certain nucleobases at certain locations (e.g., in a nucleoside opposite to a target adenosine and/or its adjacent nucleoside(s)) can provide oligonucleotides with improved properties and/or activities (e.g., adenosine editing to I). In some embodiments, a useful nucleobase is or comprises Ring BA as described herein. In some embodiments, a nucleobase in a nucleoside is or comprises Ring BA which has the structure of BA-I, BA-I-a, BA-I-b, BA-II, BA-II-a, BA-II-b, BA-III, BA-III-a, BA-III-b, BA-IV, BA-IV-a, BA-IV-b, BA-V, BA-V-a, BA-V-b, or BA-VI, or a tautomer of Ring BA, wherein the nucleobase is optionally substituted or protected. In some embodiments, a nucleobase is optionally substituted or protected, or optionally substituted or protected tautomer of:

| | |
|---|---|
| zdnp | 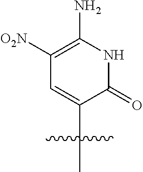 [SMILES: *c1cc([N+](=O)[O-])c(N)[nH]c1=O] |
| b001U | 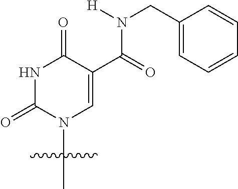 [SMILES: *n1cc(C(=O)NCc2ccccc2)c(=O)[nH]c1=O] |
| b002U | 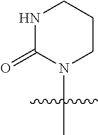 [SMILES: *N1CCCNC1=O] |
| b003U | 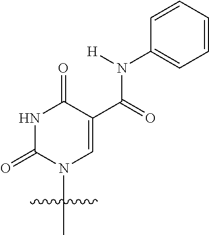 [SMILES: *n1cc(C(=O)Nc2ccccc2)c(=O)[nH]c1=O] |
| b004U | 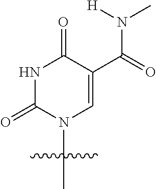 [SMILES: *n1cc(C(=O)NC)c(=O)[nH]c1=O] |
| b005U | 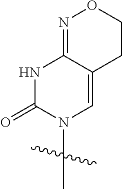 [SMILES: *N1C=C2CCON=C2NC1=O] |
| b006U | 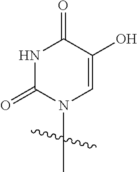 [SMILES: *n1cc(O)c(=O)[nH]c1=O] |
| b008U | 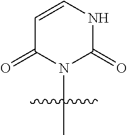 [SMILES: *n1c(=O)cc[nH]c1=O] |
| b002A | 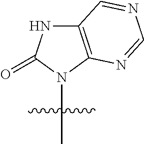 [SMILES: *n1c(=O)[nH]c2cncnc21] |
| b001G | 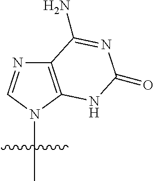 [SMILES: *n1cnc2c(N)nc(=O)[nH]c21] |
| b007U | 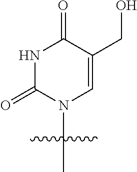 [SMILES: *n1cc(CO)c(=O)[nH]c1=O] |
| b001A | 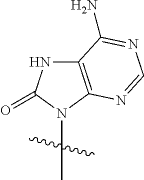 [SMILES: *n1c(=O)[nH]c2c(N)ncnc21] |
| b001C | 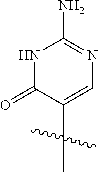 [SMILES: *c1cnc(N)[nH]c1=O] |
| b002C | 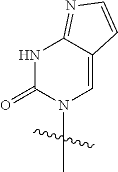 [SMILES: *n1cc2ccnc-2[nH]c1=O] |
| b003C | 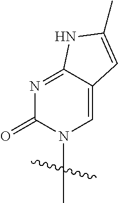 [SMILES: *n1cc2cc(C)[nH]c2nc1=O] |
| b002I | 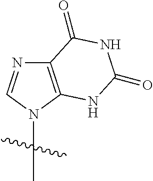 [SMILES: *n1cnc2c(=O)[nH]c(=O)[nH]c21] |
| b003I | 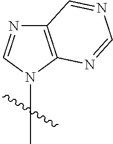 [SMILES: *n1cnc2cncnc21] |
| b009U | 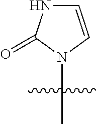 [SMILES: *n1cc[nH]c1=O] |
| b003A | 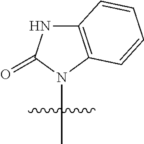 [SMILES: *n1c(=O)[nH]c2ccccc21] |

In some embodiments, the present disclosure provides oligonucleotides comprising one or more such nucleobases. In some embodiments, the present disclosure provides phosphoramidites comprising such nucleobases. In some embodiments, phosphoramidites are CED phosphoramidites. In some embodiments, phosphoramidites comprise chiral auxiliary moieties as described herein (e.g., with P forming bonds to O and N). In some embodiments, R^{NS} comprises such a nucleobase. In some embodiments, nucleobases are protected for oligonucleotide synthesis.

In some embodiments, a nucleobase is optionally substituted 2AP (2-amino purine,

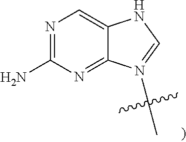

or DAP (2,6-diamino purine,

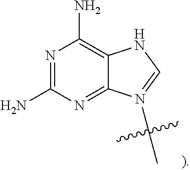

In some embodiments, a nucleobase is optionally substituted 2AP. In some embodiments, a nucleobase is optionally substituted DAP. In some embodiments, a nucleobase is 2AP. In some embodiments, a nucleobase is DAP.

As appreciated by those skilled in the art, various nucleobases are known in the art and can be utilized in accordance with the present disclosure, e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, WO 2019/032612, and/or WO 2020/191252, the sugar, base, and internucleotidic linkage modifications of each of which are independently incorporated herein by reference. In some embodiments, nucleobases are protected and useful for oligonucleotide synthesis.

In some embodiments, a nucleobase is a natural nucleobase or a modified nucleobase derived from a natural nucleobase. Examples include uracil, thymine, adenine, cytosine, and guanine optionally having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Certain examples of modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313. In some embodiments, a modified nucleobase is substituted uracil, thymine, adenine, cytosine, or guanine. In some embodiments, a modified nucleobase is a functional replacement, e.g., in terms of hydrogen bonding and/or base pairing, of uracil, thymine, adenine, cytosine, or guanine. In some embodiments, a nucleobase is optionally substituted uracil, thymine, adenine, cytosine, 5-methylcytosine, or guanine. In some embodiments, a nucleobase is uracil, thymine, adenine, cytosine, 5-methylcytosine, or guanine.

In some embodiments, a provided oligonucleotide comprises one or more 5-methylcytosine. In some embodiments, the present disclosure provides an oligonucleotide whose base sequence is disclosed herein, e.g., in Table 1, wherein each T may be independently replaced with U and vice versa, and each cytosine is optionally and independently replaced with 5-methylcytosine or vice versa. As appreciated by those skilled in the art, in some embodiments, 5mC may be treated as C with respect to base sequence of an oligonucleotide—such oligonucleotide comprises a nucleobase modification at the C position (e.g., see various oligonucleotides in Table 1). In description of oligonucleotides, typically unless otherwise noted, nucleobases, sugars and internucleotidic linkages are non-modified.

In some embodiments, a modified base is optionally substituted adenine, cytosine, guanine, thymine, or uracil, or a tautomer thereof. In some embodiments, a modified nucleobase is a modified adenine, cytosine, guanine, thymine or uracil, modified by one or more modifications by which:

a nucleobase is modified by one or more optionally substituted groups independently selected from acyl, halogen, amino, azide, alkyl, alkenyl, alkynyl, aryl, heteroalkyl, heteroalkenyl, heteroalkynyl, heterocyclyl, heteroaryl, carboxyl, hydroxyl, biotin, avidin, streptavidin, substituted silyl, and combinations thereof;

one or more atoms of a nucleobase are independently replaced with a different atom selected from carbon, nitrogen and sulfur;

one or more double bonds in a nucleobase are independently hydrogenated; or

one or more aryl or heteroaryl rings are independently inserted into a nucleobase.

In some embodiments, a base is optionally substituted A, T, C, G or U, wherein one or more —NH₂ are independently and optionally replaced with —C(-L-R¹)₃, one or more —NH— are independently and optionally replaced with —C(-L-R¹)₂—, one or more ═N— are independently and optionally replaced with —C(-L-R¹)—, one or more ═CH— are independently and optionally replaced with ═N—, and one or more ═O are independently and optionally replaced with ═S, ═N(-L-R¹), or ═C(-L-R¹)₂, wherein two or more -L-R¹ are optionally taken together with their intervening atoms to form a 3-30 membered bicyclic or polycyclic ring having 0-10 heteroatom ring atoms. In some embodiments, a modified base is optionally substituted A, T, C, G or U, wherein one or more —NH₂ are independently and optionally replaced with —C(-L-R¹)₃, one or more —NH— are independently and optionally replaced with —C(-L-R¹)₂—, one or more ═N— are independently and optionally replaced with —C(-L-R¹)—, one or more ═CH— are independently and optionally replaced with ═N—, and one or more ═O are independently and optionally replaced with ═S, ═N(-L-R¹), or ═C(-L-R¹)₂, wherein two or more -L-R¹ are optionally taken together with their intervening atoms to form a 3-30 membered bicyclic or polycyclic ring having 0-10 heteroatom ring atoms, wherein the modified base is different than the natural A, T, C, G and U. In some embodiments, a base is optionally substituted A, T, C, G or U. In some embodiments, a modified base is substituted A, T, C, G or U, wherein the modified base is different than the natural A, T, C, G and U.

In some embodiments, a modified nucleobase is a modified nucleobase known in the art, e.g., WO2017/210647. In some embodiments, modified nucleobases are expanded-size nucleobases in which one or more aryl and/or heteroaryl rings, such as phenyl rings, have been added. Certain examples of modified nucleobases, including nucleobase replacements, are described in the Glen Research catalog (Glen Research, Sterling, Va.); Krueger A T et al., Acc. Chem. Res., 2007, 40, 141-150; Kool, ET, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; or Hirao, I., Curr. Opin. Chem. Biol., 2006, 10, 622-627. In some embodiments, an expanded-size nucleobase is an expanded-size nucleobase described in, e.g., WO2017/210647. In some embodiments, modified nucleobases are moieties such as corrin- or porphyrin-derived rings. Certain porphyrin-derived base replacements have been described in, e.g., Morales-Rojas, H and Kool, ET, Org. Lett., 2002, 4, 4377-4380. In some embodiments, a porphyrin-derived ring is a porphyrin-derived ring described in, e.g., WO2017/219647. In some embodiments, a modified nucleobase is a modified nucleobase described in, e.g., WO2017/219647. In some embodiments, a modified nucleobase is fluorescent. Examples of such fluorescent modified nucleobases include phenanthrene, pyrene, stillbene, isoxanthine, isozanthopterin, terphenyl, terthiophene, benzoterthiophene, coumarin, lumazine, tethered stillbene, benzo-uracil, naphtho-uracil, etc., and those described in e.g., WO2017/210647. In some embodiments, a nucleobase or modified nucleobase is selected from: C5-propyne T, C5-propyne C, C5-Thiazole, phenoxazine, 2-thio-thymine, 5-triazolylphenyl-thymine, diaminopurine, and N2-aminopropylguanine.

In some embodiments, a modified nucleobase is selected from 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. In some embodiments, modified nucleobases are tricyclic pyrimidines, such as 1,3-diazaphenoxazine-2-one,1,3-diazaphenothiazine-2-one or 9-(2-aminoethoxy)-1,3-diazaphenoxazine-2-one (G-clamp). In some embodiments, modified nucleobases are those in which the purine or pyrimidine base is replaced with other heterocycles, for example, 7-deaza-adenine, 7-deazaguanosine, 2-aminopyridine or 2-pyridone. In some embodiments, modified nucleobases are those disclosed in U.S. Pat. No. 3,687,808, The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; or in Chapters 6 and 15, Antisense Drug Technology, Crooke S. T., Ed., CRC Press, 2008, 163-166 and 442-443.

In some embodiments, modified nucleobases and methods thereof are those described in US 20030158403, U.S. Pat. Nos. 3,687,808, 4,845,205, 5,130,302, 5,134,066, 5,175,273, 5,367,066, 5,432,272, 5,434,257, 5,457,187, 5,459,255, 5,484,908, 5,502,177, 5,525,711, 5,552,540, 5,587,469, 5,594,121, 5,596,091, 5,614,617, 5,645,985, 5,681,941, 5,750,692, 5,763,588, 5,830,653, or U.S. Pat. No. 6,005,096.

In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One example of a universal base is 3-nitropyrrole.

In some embodiments, nucleosides that can be utilized in provided technologies comprise modified nucleobases and/or modified sugars, e.g., 4-acetylcytidine; 5-(carboxyhydroxylmethyl)uridine; 2′-O -methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-me thoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, a nucleobase, e.g., a modified nucleobase comprises one or more biomolecule binding moieties such as e.g., antibodies, antibody fragments, biotin, avidin, streptavidin, receptor ligands, or chelating moieties. In other embodiments, a nucleobase is 5-bromouracil, 5-iodouracil, or 2,6-diaminopurine. In some embodiments, a nucleobase comprises substitution with a fluorescent or biomolecule binding moiety. In some embodiments, a substituent is a fluorescent moiety. In some embodiments, a substituent is biotin or avidin.

Certain examples of nucleobases and related methods are described in U.S. Pat. Nos. 3,687,808, 4,845,205, 513,030, 5,134,066, 5,175,273, 5,367,066, 5,432,272, 5,457,187, 5,457,191, 5,459,255, 5,484,908, 5,502,177, 5,525,711, 5,552,540, 5,587,469, 5,594,121, 5,596,091, 5,614,617, 5,681,941, 5,750,692, 6,015,886, 6,147,200, 6,166,197, 6,222,025, 6,235,887, 6,380,368, 6,528,640, 6,639,062, 6,617,438, 7,045,610, 7,427,672, US or U.S. Pat. No. 7,495,088.

In some embodiments, an oligonucleotide comprises a nucleobase, sugar, nucleoside, and/or internucleotidic linkage which is described in any of: Gryaznov, S; Chen, J.-K. J. Am. Chem. Soc. 1994, 116, 3143; Hendrix et al. 1997 Chem. Eur. J. 3: 110; Hyrup et al. 1996 Bioorg. Med. Chem. 4: 5; Jepsen et al. 2004 Oligo. 14: 130-146; Jones et al. J. Org. Chem. 1993, 58, 2983; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Mesmaeker et al. Angew. Chem., Int. Ed. Engl. 1994, 33, 226; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Pallan et al. 2012 Chem. Comm. 48: 8195-8197; Petersen et al. 2003 TRENDS Biotech. 21: 74-81; Rajwanshi et al. 1999 Chem. Commun. 1395-1396; Schultz et al. 1996 Nucleic Acids Res. 24: 2966; Seth et al. 2009 J. Med. Chem. 52: 10-13; Seth et al. 2010 J. Med. Chem. 53: 8309-8318; Seth et al. 2010 J. Org. Chem. 75: 1569-1581; Seth et al. 2012 Bioo. Med. Chem. Lett. 22: 296-299; Seth et al. 2012 Mol. Ther-Nuc. Acids. 1, e47; Seth, Punit P; Siwkowski, Andrew; Allerson, Charles R; Vasquez, Guillermo; Lee, Sam; Prakash, Thazha P; Kinberger, Garth; Migawa, Michael T; Gaus, Hans; Bhat, Balkrishen; et al. From Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Ts'o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; WO 2007090071; or WO 2016/079181.

In some embodiments, an oligonucleotide comprises a modified nucleobase, nucleoside or nucleotide which is described in any of: Feldman et al. 2017 J. Am. Chem. Soc. 139: 11427-11433, Feldman et al. 2017 Proc. Natl. Acad. Sci. USA 114: E6478-E6479, Hwang et al. 2009 Nucl. Acids Res. 37: 4757-4763, Hwang et al. 2008 J. Am. Chem. Soc. 130: 14872-14882, Lavergne et al. 2012 Chem. Eur. J. 18: 1231-1239, Lavergne et al. 2013 J. Am. Chem. Soc. 135: 5408-5419, Ledbetter et al. 2018 J. Am. Chem. Soc. 140: 758-765, Malyshev et al. 2009 J. Am. Chem. Soc. 131: 14620-14621, Seo et al. 2009 Chem. Bio. Chem. 10: 2394-2400, e.g., d3FB, d2Py analogs, d2Py, d3MPy, d4MPy, d5MPy, d34DMPy, d35DMPy, d45DMPy, d5FM, d5PrM, d5SICS, dFEMO, dMMO2, dNaM, dNM01, dTPT3, nucleotides with 2′-azido, 2′-chloro, 2′-amino or arabinose sugars, isocarbostiryl-, napthyl- and azaindole-nucleotides, and modifications and derivatives and functionalized versions thereof, e.g., those in which the sugar comprises a 2′-modification and/or other modification, and dMMO2 derivatives with meta-chlorine, -bromine, -iodine, -methyl, or -propinyl substituents.

In some embodiments, a nucleobase comprises at least one optionally substituted ring which comprises a heteroatom ring atom. In some embodiments, a nucleobase comprises at least one optionally substituted ring which comprises a nitrogen ring atom. In some embodiments, such a ring is aromatic. In some embodiments, a nucleobase is bonded to a sugar through a heteroatom. In some embodiments, a nucleobase is bonded to a sugar through a nitrogen atom. In some embodiments, a nucleobase is bonded to a sugar through a ring nitrogen atom.

In some embodiments, an oligonucleotide comprises a nucleobase or modified nucleobase as described in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the bases and modified nucleobases of each of which are independently incorporated herein by reference.

In some embodiments, a nucleobase is an optionally substituted purine base residue. In some embodiments, a nucleobase is a protected purine base residue. In some embodiments, a nucleobase is an optionally substituted adenine residue. In some embodiments, a nucleobase is a protected adenine residue. In some embodiments, a nucleobase is an optionally substituted guanine residue. In some embodiments, a nucleobase is a protected guanine residue. In some embodiments, a nucleobase is an optionally substituted cytosine residue. In some embodiments, a nucleobase is a protected cytosine residue. In some embodiments, a nucleobase is an optionally substituted thymine residue. In some embodiments, a nucleobase is a protected thymine residue. In some embodiments, a nucleobase is an optionally substituted uracil residue. In some embodiments, a nucleobase is a protected uracil residue. In some embodiments, a nucleobase is an optionally substituted 5-methylcytosine residue. In some embodiments, a nucleobase is a protected 5-methylcytosine residue.

In some embodiments, a provided oligonucleotide comprises a modified nucleobase described in, e.g., U.S. Pat. Nos. 5,552,540, 6,222,025, 6,528,640, 4,845,205, 5,681,941, 5,750,692, 6,015,886, 5,614,617, 6,147,200, 5,457,187, 6,639,062, 7,427,672, 5,459,255, 5,484,908, 7,045,610, 3,687,808, 5,502,177, 5,525,711 6,235,887, 5,175,273, 6,617,438, 5,594,121, 6,380,368, 5,367,066, 5,587,469, 6,166,197, 5,432,272, 7,495,088, 5,134,066, or 5,596,091.

In some embodiments, a nucleobase is a protected base residue as used in oligonucleotide preparation. In some embodiments, a nucleobase is a base residue illustrated in US 2011/0294124, US 2015/0211006, US 2015/0197540, WO 2015/107425, WO 2017/192679, WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the base residues of each of which are independently incorporated herein by reference.

### Sugars

Various sugars, including modified sugars, can be utilized in accordance with the present disclosure. In some embodiments, the present disclosure provides sugar modifications and patterns thereof optionally in combination with other structural elements (e.g., internucleotidic linkage modifications and patterns thereof, pattern of backbone chiral centers thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In some embodiments, a sugar, e.g., various sugars in many oligonucleotides in Table 1 (unless otherwise notes), is a natural DNA sugar (in DNA nucleic acids or oligonucleotides, having the structure of

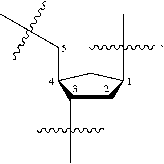

wherein a nucleobase is attached to the 1′ position, and the 3′ and 5′ positions are connected to internucleotidic linkages (as appreciated by those skilled in the art, if at the 5′-end of oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a natural RNA sugar (in RNA nucleic acids or oligonucleotides, having the structure of

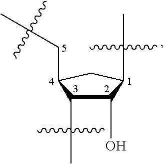

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a modified sugar in that it is not a natural DNA sugar or a natural RNA sugar. Among other things, modified sugars may provide improved stability. In some embodiments, modified sugars can be utilized to alter and/or optimize one or more hybridization characteristics. In some embodiments, modified sugars can be utilized to alter and/or optimize target nucleic acid recognition. In some embodiments, modified sugars can be utilized to optimize Tm. In some embodiments, modified sugars can be utilized to improve oligonucleotide activities.

Sugars can be bonded to internucleotidic linkages at various positions. As non-limiting examples, internucleotidic linkages can be bonded to the 2′, 3′, 4′ or 5′ positions of sugars. In some embodiments, as most commonly in natural nucleic acids, an internucleotidic linkage connects with one sugar at the 5′ position and another sugar at the 3′ position unless otherwise indicated.

In some embodiments, a sugar is an optionally substituted natural DNA or RNA sugar. In some embodiments, a sugar is optionally substituted

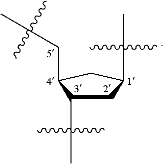

In some embodiments, the 2′ position is optionally substituted. In some embodiments, a sugar is

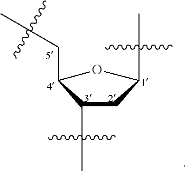 [SMILES: *C[C@H]1O[C@@H](*)C[C@H]1*]

In some embodiments, a sugar has the structure of

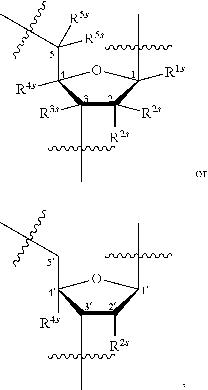

wherein each of R^{1s}, R^{2s}, R^{3s}, R^{4s}, and R^{5s} is independently —H, a suitable substituent or suitable sugar modification (e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, WO 2019/032612, and/or WO 2020/191252, the substituents, sugar modifications, descriptions of R^{1s}, R^{2s}, R^{3s}, R^{4s}, and R^{5s}, and modified sugars of each of which are independently incorporated herein by reference). In some embodiments, each of R^{1s}, R^{2s}, R^{3s}, R^{4s}, and R^{5s} is independently R^{s}, wherein each R^{s} is independently —F, —Cl, —Br, —I, —CN, —N₃, —NO, —NO₂, -L^{s}-R′, -L^{s}-OR′, -L^{s}-SR′, -L^{s}-N(R′)₂, —O-L^{s}-OR′, —O-L^{s}-SR′, or —O-L^{s}-N(R′)₂, wherein each R′ is independently as described herein, and each L^{s} is independently a covalent bond or optionally substituted bivalent C₁₋₆ aliphatic or heteroaliphatic having 1-4 heteroatoms; or two R^{s} are taken together to form a bridge -L^{s}-. In some embodiments, R′ is optionally substituted C₁₋₁₀ aliphatic. In some embodiments, a sugar has the structure of

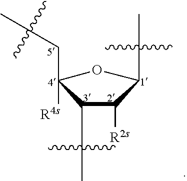

Various such sugars are utilized in Table 1. In some embodiments, a sugar has the structure of

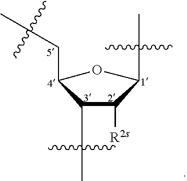

In some embodiments, a 2′-modified sugar has the structure of

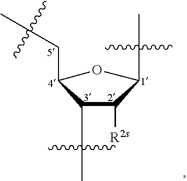

wherein R^{2s} is a 2′-modification. In some embodiments, a sugar has the structure of

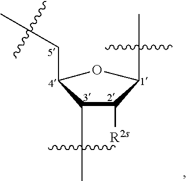

wherein R^{2s} is —H, halogen, or —OR, wherein R is optionally substituted C₁₋₆ aliphatic. In some embodiments, R^{2s} is —H. In some embodiments, R^{2s} is —F. In some embodiments, R^{2s} is —OMe. In some embodiments, a modified nucleoside is mA, mT, mC, m5mC, mG, mU, etc., in which R^{2s} is —OMe. In some embodiments, R^{2s} is —OCH₂CH₂OMe. In some embodiments, a modified nucleoside is Aeo, Teo, Ceo, m5Ceo, Geo, Ueo, etc., in which R^{2s} is —OCH₂CH₂OMe. In some embodiments, a 2′-F modified sugar has the structure of

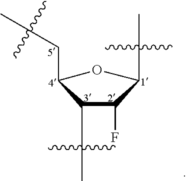

In some embodiments, a 2′-OMe modified sugar has the structure of

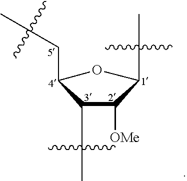

In some embodiments, a sugar has the structure of

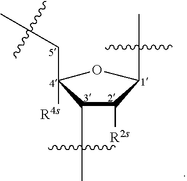

wherein R^{2s} and R^{4s} are taken together to form -L^{s}-, wherein L^{s} is a covalent bond or optionally substituted bivalent C₁₋₆ aliphatic or heteroaliphatic having 1-4 heteroatoms. In some embodiments, each heteroatom is independently selected from nitrogen, oxygen or sulfur). In some embodiments, L^{s} is optionally substituted C2-O—CH₂—C4. In some embodiments, L^{s} is C2-O—CH₂—C4. In some embodiments, L^{s} is C2-O—(R)—CH(CH₂CH₃)—C4. In some embodiments, L^{s} is C2-O—(S)—CH(CH₂CH₃)—C4.

In some embodiments, a nucleoside comprising a modified sugar has the structure of

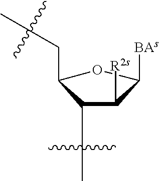

or a salt form thereof, wherein BA^{s} is —H or an optionally substituted or protected nucleobase (e.g., BA), and R^{2s} is as described herein. In some embodiments, R^{2s} is —OH, halogen, or optionally substituted C₁-C₆ alkoxy. In some embodiments, BA^{s} is —H. In some embodiments, BA^{s} is an optionally substituted or protected nucleobase. In some embodiments, BA^{s} is BA. In some embodiments, R^{2s} is —F. In some embodiments, a nucleoside comprising a modified sugar has the structure of

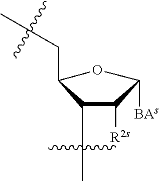

or a salt form thereof, wherein each variable is independently as described herein. In some embodiments, R^{2s} is —H, —OH, halogen, or optionally substituted C₁-C₆ alkoxy. In some embodiments, R^{2s} is —H. In some embodiments, R^{2s} is —F. In some embodiments, a nucleoside comprising a modified sugar has the structure of

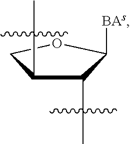

wherein each variable is as described herein. In some embodiments, a nucleoside comprising a modified sugar has the structure of

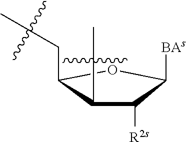

or a salt form thereof, wherein each variable is independently as described herein. In some embodiments, R^{2s} is —H, —OH, halogen, or optionally substituted C₁-C₆ alkoxy. In some embodiments, R^{2s} is —H. In some embodiments, R^{2s} is —F. In some embodiments, a nucleoside comprising a modified sugar has the structure of

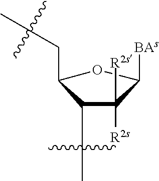

or a salt form thereof, wherein R^{2s} is R^{s}, and each of R^{s}, R^{2s} and BAS is independently as described herein. In some embodiments, each of R^{2s} and R^{2s}′ is independently —H, —OH, halogen, or optionally substituted C₁-C₆ alkoxy. In some embodiments, R^{2s} is —H. In some embodiments, R^{2s} is —OH. In some embodiments, R^{2s} is halogen. In some embodiments, R^{2s} is —F. In some embodiments, R^{2s} is optionally substituted C₁-C₆ alkoxy. In some embodiments, R^{2s′} is —H. In some embodiments, R^{2s′} is —OH. In some embodiments, R^{2s′} is halogen. In some embodiments, R^{2s} is —F. In some embodiments, R^{2s′} is optionally substituted C₁-C₆ alkoxy. In some embodiments, BA^{s} is —H. In some embodiments, BA^{s} is an optionally substituted or protected nucleobase. In some embodiments, BA^{s} is BA. In some embodiments, nucleobases such as BA are optionally substituted or protected for oligonucleotide synthesis. Certain such nucleosides including sugars and nucleobases and uses thereof are described in WO 2020/154342. In some embodiments, oligonucleotides comprise such nucleosides. In some embodiments, phosphoramidites comprise such nucleosides (in some embodiments, one connecting site (e.g., a —CH₂— connecting site) is bonded to an optionally substituted —OH, e.g., (-ODMTr), and one connecting site (e.g., a ring connecting site) is bonded to O which is also bonded to P of a phosphoramidite). In some embodiments, one or more or each of a 5′ immediate nucleoside (e.g., N₁), an opposite nucleoside (N₀) and a 3′ immediate nucleoside (e.g., N₋₁) is independently such a nucleoside.

In some embodiments, a nucleoside comprising a modified sugar has the structure of

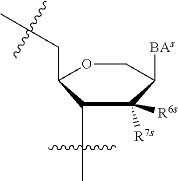

or a salt form thereof, wherein each of R^{6s} and R^{7s} is independently R^{s}, BA^{s} is —H or an optionally substituted or protected nucleobase (e.g., BA), and R^{s} is independently as described herein. In some embodiments, R^{6s} is —H, —OH or halogen, and R^{7s} is —H, —OH, halogen or optionally substituted C₁-C₆ alkoxy. In some embodiments, BA^{s} is —H. In some embodiments, BA^{s} is an optionally substituted or protected nucleobase. In some embodiments BA^{s} is BA. In some embodiments, a nucleoside comprising a modified sugar has the structure of

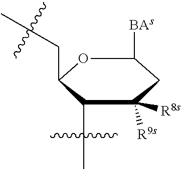

or a salt form thereof, wherein each of R^{8s} and R^{9s} is independently R^{s}, and each of R^{s} and BA^{s} is independently as described herein. In some embodiments, R^{8s} is —H or halogen, and R^{9s} is —H, —OH, halogen, or optionally substituted C₁-C₆ alkoxy. In some embodiments, a nucleoside comprising a modified sugar has the structure of

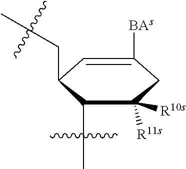

or a salt form thereof, wherein each of R^{10s} and R^{11s} is independently R^{s}, and each of R^{s} and BA^{s} is independently as described herein. In some embodiments, R^{10s} is —H or halogen, and R^{11s} is —H, —OH, halogen, or optionally substituted C₁-C₆ alkoxy. In some embodiments, a nucleoside comprising a modified sugar has the structure of

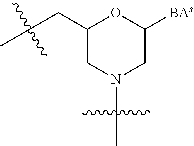

or a salt form thereof, wherein BA^{s} is as described herein. Those skilled in the art appreciate that in some embodiments, the nitrogen may be directly bonded to linkage phosphorus. In some embodiments, a halogen is —F. In some embodiments, BA^{s} is —H. In some embodiments, BA^{s} is an optionally substituted or protected nucleobase. In some embodiments, BA^{s} is BA. In some embodiments, nucleobases such as BA are optionally substituted or protected for oligonucleotide synthesis. Certain such nucleosides including sugars and nucleobases and uses thereof are described in WO 2020/154343. In some embodiments, oligonucleotides comprise such nucleosides. In some embodiments, phosphoramidites comprise such nucleosides (in some embodiments, one connecting site (e.g., a —CH₂-connecting site) is bonded to an optionally substituted —OH, e.g., -ODMTr, and one connecting site (e.g., a ring connecting site) is bonded to P of a phosphoramidite (e.g., when the connecting ring atom is N) or to O which is also bonded to P of a phosphoramidite(e.g., when the connecting ring atom is C)). In some embodiments, one or more or each of a 5′ immediate nucleoside (e.g., N₁), an opposite nucleoside (N₀) and a 3′ immediate nucleoside (e.g., N₋₁) is independently such a nucleoside.

In some embodiments, a nucleoside comprising a modified sugar has the structure of

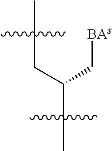

or a salt form thereof, wherein each variable is as described herein. In some embodiments, a nucleoside comprising a modified sugar has the structure of

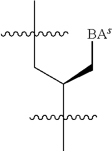

or a salt form thereof, wherein each variable is as described herein. In some embodiments, a nucleoside comprising a modified sugar has the structure of

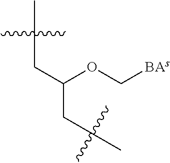

or a salt form thereof, wherein each variable is as described herein. In some embodiments, a nucleoside comprising a modified sugar has the structure of

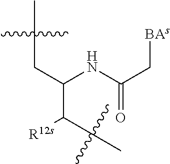

or a salt form thereof, wherein R^{12s} is R^{s}, and each of R^{s} and BA^{s} is independently as described herein. In some embodiments, R^{12s} is —H, —OH, halogen, optionally substituted C₁₋₆ alkyl, optionally substituted C₁₋₆ heteroalkyl, or optionally substituted C₁₋₆ alkoxy. In some embodiments, a halogen is —F. In some embodiments, a nucleoside comprising a modified sugar has the structure of

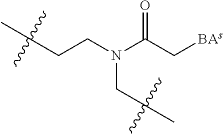

or a salt form thereof, wherein each variable is as described herein. In some embodiments, a nucleotide comprising a modified sugar has the structure of

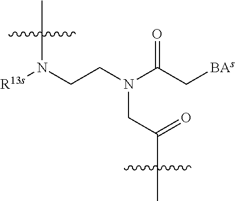

or a salt form thereof, wherein R^{13s} is R^{s}, and each of R^{s} and BA^{s} is independently as described herein. In some embodiments, R^{13s} is —H or optionally substituted C₁-C₆ alkyl. In some embodiments, a nucleoside comprising a modified sugar has the structure

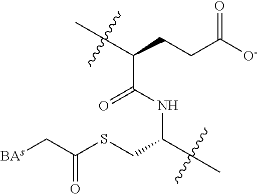

or a salt form thereof, wherein each variable is as described herein. In some embodiments, a nucleotide comprising a modified sugar has the structure of

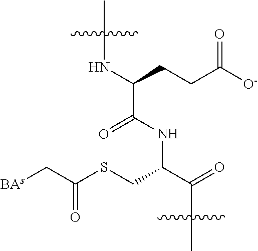

or a salt form thereof, wherein each variable is as described herein. In some embodiments, a linkage is an amide linkage. In some embodiments, BA^{s} is —H. In some embodiments, BA^{s} is an optionally substituted or protected nucleobase. In some embodiments, BA^{s} is BA. In some embodiments, nucleobases such as BA are optionally substituted or protected for oligonucleotide synthesis. Certain such nucleosides and nucleotides including sugars and nucleobases and uses thereof are described in WO 2020/154344. In some embodiments, oligonucleotides comprise such nucleosides. In some embodiments, oligonucleotides comprise such nucleosides (in some embodiments, one connecting site (e.g., a —CH₂— connecting site) is bonded to an optionally substituted —OH, e.g., (-ODMTr), and one connecting site (e.g., a ring connecting site) is bonded to O which is also bonded to P of a phosphoramidite. In some embodiments, one or more or each of a 5′ immediate nucleoside (e.g., N₁), an opposite nucleoside (No) and a 3′ immediate nucleoside (e.g., N₋₁) is independently such a nucleoside.

In some embodiments, a sugar is an acyclic sugar, e.g. a UNA sugar. In some embodiments, a sugar is optionally substituted

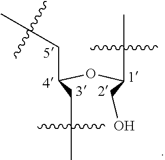

In some embodiments, the 2′ position is optionally substituted. In some embodiments, a sugar is

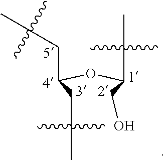

In some embodiments, a sugar has the structure of

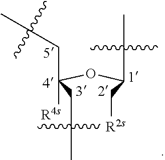

In some embodiments, R^{2s} is —OH.

In some embodiments, each of R^{1s}, R^{2s}, R^{3s}, R^{4s}, and R^{5s} is independently R^{s}, wherein R^{s} is independently —H, halogen, —CN, —N₃, —NO, —NO₂, -L^{s}-R′, -L^{s}-Si(R′)₃, -L^{s}-OR′, -L^{s}-SR′, -L^{s}-N(R′)₂, —O-L^{s}-R′, —O-L^{s}-Si(R)₃, —O-L^{s}-OR′, —O-L^{s}-SR′, or —O-L^{s}-N(R′)₂; wherein L^{s} is L^{B} as described herein, and each other variable is independently as described herein. In some embodiments, each of R^{1s} and R^{2s} is independently R^{s}. In some embodiments, R^{s} is —H. In some embodiments, R^{s} is not —H. In some embodiments, L^{s} is a covalent bond. In some embodiments, each of R^{2s} and R^{4s} are independently —H, —F, —OR, —N(R)₂. In some embodiments, R^{2s} is —H, —F, —OR, —N(R)₂. In some embodiments, R^{4s} is —H. In some embodiments, R^{2s} and R^{4s} form 2′-O-L^{s}-, wherein L^{s} is optionally substituted C₁₋₆ alkylene. In some embodiments, L^{s} is optionally substituted —CH₂—. In some embodiments, L^{s} is optionally substituted —CH₂—.

In some embodiments, R is hydrogen. In some embodiments, R is not hydrogen. In some embodiments, R is an optionally substituted group selected from C₁₋₁₀ aliphatic, C₁₋₁₀ heteroaliphatic having 1-10 heteroatoms independently selected from oxygen, nitrogen, sulfur, phosphorus and silicon, C₆₋₂₀ aryl, a 5-20 membered heteroaryl ring having 1-10 heteroatoms independently selected from oxygen, nitrogen, sulfur, phosphorus and silicon, and a 3-20 membered heterocyclic ring having 1-10 heteroatoms independently selected from oxygen, nitrogen, sulfur, phosphorus and silicon.

In some embodiments, R is optionally substituted C₁₋₃₀ aliphatic. In some embodiments, R is optionally substituted C₁₋₂₀ aliphatic. In some embodiments, R is optionally substituted C₁₋₁₅ aliphatic. In some embodiments, R is optionally substituted C₁₋₁₀ aliphatic. In some embodiments, R is optionally substituted C₁₋₆ aliphatic. In some embodiments, R is optionally substituted C₁₋₆ alkyl. In some embodiments, R is optionally substituted hexyl, pentyl, butyl, propyl, ethyl or methyl. In some embodiments, R is optionally substituted hexyl. In some embodiments, R is optionally substituted pentyl. In some embodiments, R is optionally substituted butyl. In some embodiments, R is optionally substituted propyl. In some embodiments, R is optionally substituted ethyl. In some embodiments, R is optionally substituted methyl. In some embodiments, R is hexyl. In some embodiments, R is pentyl. In some embodiments, R is butyl. In some embodiments, R is propyl. In some embodiments, R is ethyl. In some embodiments, R is methyl. In some embodiments, R is isopropyl. In some embodiments, R is n-propyl. In some embodiments, R is tert-butyl. In some embodiments, R is sec-butyl. In some embodiments, R is n-butyl. In some embodiments, R is —(CH₂)₂OCH₃.

In some embodiments, R is optionally substituted phenyl. In some embodiments, R is phenyl.

In some embodiments, R^{2s} is a 2′-modification as described in the present disclosure, and R^{4s} is —H. In some embodiments, R^{2s} is —OR, wherein R is not hydrogen. In some embodiments, R^{2s} is —F. In some embodiments, R^{2s} is —OMe. In some embodiments, R^{2s} is —OCH₂CH₂CH₃, e.g., in various Xₑₒ utilized in Table 1 (X being m5C, T, G, A, etc.). In some embodiments, R^{2s} is selected from —H, —F, and —OR, wherein R is optionally substituted C₁₋₆ alkl. In some embodiments, R^{2s} is selected from —H, —F, and —OMe.

In some embodiments, a sugar is a bicyclic sugar, e.g., sugars wherein R^{2s} and R^{4s} are taken to form an optionally substituted ring as described in the present disclosure. In some embodiments, a sugar is selected from LNA sugars, BNA sugars, cEt sugars, etc. In some embodiments, a bridge is between the 2′ and 4′-carbon atoms (corresponding to R^{2s} and R^{4s} taken together with their intervening atoms to form an optionally substituted ring as described herein). In some embodiments, a bridge is 2′-L^{a}-L^{b}-4′, wherein La is —O—, —S— or N(R), and L^{b} is an optionally substituted C₁₋₄ bivalent aliphatic chain, e.g., methylene.

In some embodiments, a sugar is a 2′-OMe, 2′-MOE, 2′-F, a LNA (locked nucleic acid) sugar, an ENA (ethylene bridged nucleic acid) sugar, a BNA(NMe) (Methylamino bridged nucleic acid) sugar, 2′-F ANA (2′-F arabinose), alpha-DNA (alpha-D-ribose), 2′/5′ ODN (e.g., 2′/5′ linked oligonucleotide), Inv (inverted sugar, e.g., inverted desoxyribose), AmR (Amino-Ribose), ThioR (Thio-ribose), HNA (hexose nucleic acid), CeNA (cyclohexene nucleic acid), or MOR (Morpholino) sugar.

Those skilled in the art after reading the present disclosure will appreciate that various types of sugar modifications are known and can be utilized in accordance with the present disclosure. In some embodiments, a sugar modification is a 2′-modification (e.g., R^{2s}). In some embodiments, a 2′-modification is 2′-F. In some embodiments, a 2′-modification is 2′-OR, wherein R is not hydrogen. In some embodiments, a 2′-modification is 2′-OR, wherein R is optionally substituted C₁₋₆ aliphatic. In some embodiments, a 2′-modification is 2′-OR, wherein R is optionally substituted C₁₋₆ alkyl. In some embodiments, a 2′-modification is 2′-OMe. In some embodiments, a 2′-modification is 2′-MOE. In some embodiments, a 2′-modification is —O-L^{b}- or -L^{b}-L^{b}- which connects the 2′-carbon of a sugar moiety to another carbon of a sugar moiety. In some embodiments, a 2′-modification is 2′-O-L-4′ or 2′-O-L^{b}-4′ or 2′-L^{b}-L^{b}-4′ which connects the 2′-carbon of a sugar moiety to the 4′-carbon of a sugar moiety. In some embodiments, a 2′-modification is S-cEt. In some embodiments, a modified sugar is an LNA sugar. In some embodiments, -L^{b}- is —C(R)₂—. In some embodiments, a 2′-modification is (C2-O—C(R)₂—C4), wherein each R is independently as described in the present disclosure. In some embodiments, a 2′-modification is a LNA sugar modification (C2-O—CH₂—C4). In some embodiments, a 2′-modification is (C2-O—CHR—C4), wherein R is as described in the present disclosure. In some embodiments, a 2′-modification is (C2-O—(R)—CHR—C4), wherein R is as described in the present disclosure and is not hydrogen. In some embodiments, a 2′-modification is (C2-O—(S)—CHR—C4), wherein R is as described in the present disclosure and is not hydrogen. In some embodiments, R is optionally substituted C₁₋₆ aliphatic. In some embodiments, R is optionally substituted C₁₋₆ alkyl. In some embodiments, R is unsubstituted C₁₋₆ alkyl. In some embodiments, R is methyl. In some embodiments, R is ethyl. In some embodiments, a 2′-modification is (C2-O—CHR—C4), wherein R is optionally substituted C₁₋₆ aliphatic. In some embodiments, a 2′-modification is (C2-O—CHR—C4), wherein R is optionally substituted C₁₋₆ alkyl. In some embodiments, a 2′-modification is (C2-O—CHR—C4), wherein R is methyl. In some embodiments, a 2′-modification is (C2-O—CHR—C4), wherein R is ethyl. In some embodiments, a 2′-modification is (C2-O—(R)—CHR—C4), wherein R is optionally substituted C₁_₆ aliphatic. In some embodiments, a 2′-modification is (C2-O—(R)—CHR—C4), wherein R is optionally substituted C₁₋₆ alkyl. In some embodiments, a 2′-modification is (C2-O—(R)—CHR—C4), wherein R is methyl. In some embodiments, a 2′-modification is (C2-O—(R)—CHR—C4), wherein R is ethyl. In some embodiments, a 2′-modification is (C2-O—(S)—CHR—C4), wherein R is optionally substituted C₁₋₆ aliphatic. In some embodiments, a 2′-modification is (C2-O—(S)—CHR—C4), wherein R is optionally substituted C₁₋₆ alkyl. In some embodiments, a 2′-modification is (C2-O—(S)—CHR—C4), wherein R is methyl. In some embodiments, a 2′-modification is (C2-O—(S)—CHR—C4), wherein R is ethyl. In some embodiments, a 2′-modification is C2-O—(R)—CH(CH₂CH₃)—C4. In some embodiments, a 2′-modification is C2-O—(S)—CH(CH₂CH₃)—C4. In some embodiments, a sugar is a natural DNA sugar. In some embodiments, a sugar is a natural RNA sugar. In some embodiments, a sugar is an optionally substituted natural DNA sugar. In some embodiments, a sugar is a natural DNA sugar optionally substituted at 2′. In some embodiments, a sugar is a natural DNA sugar substituted at 2′ (2′-modification). In some embodiments, a sugar is a natural DNA sugar modified at 2′ (2′-modification).

In some embodiments, a sugar is an optionally substituted ribose or deoxyribose. In some embodiments, a sugar is an optionally modified ribose or deoxyribose, wherein one or more hydroxyl groups of the ribose or deoxyribose moiety is optionally and independently replaced by halogen, R′, —N(R′)₂, —OR′, or —SR′, wherein each R′ is as described herein. In some embodiments, a sugar is an optionally substituted deoxyribose, wherein the 2′ position of the deoxyribose is optionally substituted. In some embodiments, a sugar is an optionally substituted deoxyribose, wherein the 2′ position of the deoxyribose is optionally substituted with halogen, R′, —N(R′)₂, —OR′, or —SR′, wherein each R′ is independently described in the present disclosure. In some embodiments, a sugar is an optionally substituted deoxyribose, wherein the 2′ position of the deoxyribose is optionally substituted with halogen. In some embodiments, a sugar is an optionally substituted deoxyribose, wherein the 2′ position of the deoxyribose is optionally substituted with one or more —F. In some embodiments, a sugar is an optionally substituted deoxyribose, wherein the 2′ position of the deoxyribose is optionally substituted with —OR′, wherein each R′ is independently described in the present disclosure. In some embodiments, a sugar is an optionally substituted deoxyribose, wherein the 2′ position of the deoxyribose is optionally substituted with —OR′, wherein each R′ is independently optionally substituted C₁-C₆ aliphatic. In some embodiments, a sugar is an optionally substituted deoxyribose, wherein the 2′ position of the deoxyribose is optionally substituted with —OR′, wherein each R′ is independently an optionally substituted C₁-C₆ alkyl. In some embodiments, a sugar is an optionally substituted deoxyribose, wherein the 2′ position of the deoxyribose is optionally substituted with —OMe. In some embodiments, a sugar is an optionally substituted deoxyribose, wherein the 2′ position of the deoxyribose is optionally substituted with —O-methoxyethyl.

In some embodiments, provided oligonucleotides comprise one or more modified sugars. In some embodiments, provided oligonucleotides comprise one or more modified sugars and one or more natural sugars.

Examples of bicyclic sugars include sugars of alpha-L-methyleneoxy (4′-CH₂—O-2′) LNA, beta-D-methyleneoxy (4′-CH₂—O-2′) LNA, ethyleneoxy (4′-(CH₂)₂—O-2′) LNA, aminooxy (4′-CH₂—O—N(R)-2′) LNA, and oxyamino (4′-CH₂—N(R)—O-2′) LNA. In some embodiments, a bicyclic sugar, e.g., a LNA or BNA sugar, is sugar having at least one bridge between two sugar carbons. In some embodiments, a bicyclic sugar in a nucleoside may have the stereochemical configurations of alpha-L-ribofuranose or beta-D-ribofuranose.

In some embodiments, a bicyclic sugar may be further defined by isomeric configuration. For example, a sugar comprising a 4′-(CH₂)—O-2′ bridge may be in the alpha-L configuration or in the beta-D configuration. In some embodiments, a 4′ to 2′ bridge is a -L-4′-(CH₂)—O-2′, b-D-4′-CH₂—O-2′, 4′—(CH₂)₂—O-2′, 4′—CH₂—O—N(R′)-2′, 4′—CH₂—N(R′)—O-2′, 4′-CH(R′)—O-2′, 4′-CH(CH₃)—O-2′, 4′—CH₂—S-2′, 4′—CH₂—N(R′)-2′, 4′—CH₂—CH(R′)-2′, 4′—CH₂—CH(CH₃)-2′, and 4′-(CH₂)₃-2′, wherein each R′ is as described in the present disclosure. In some embodiments, R′ is —H, a protecting group or optionally substituted C₁-C₁₂ alkyl. In some embodiments, R′ is —H or optionally substituted C₁-C₁₂ alkyl.

In some embodiments, a bicyclic sugar is a sugar of alpha-L-methyleneoxy (4′-CH₂—O-2′) BNA, beta-D-methyleneoxy (4′-CH₂—O-2′) BNA, ethyleneoxy (4′-(CH₂)₂—O-2′) BNA, aminooxy (4′-CH₂—O—N(R)-2′) BNA, oxyamino (4′-CH₂—N(R)—O-2′) BNA, methyl(methyleneoxy) (4′-CH(CH₃)—O-2′) BNA (also referred to as constrained ethyl or cEt), methylene-thio (4′-CH₂—S-2′) BNA, methylene-amino (4′-CH₂—N(R)-2′) BNA, methyl carbocyclic (4′-CH₂—CH(CH₃)-2′) BNA, propylene carbocyclic (4′-(CH₂)₃-2′) BNA, or vinyl BNA.

In some embodiments, a sugar modification is a modification described in U.S. Pat. No. 9,006,198. In some embodiments, a modified sugar is described in U.S. Pat. No. 9,006,198. In some embodiments, a sugar modification is a modification described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, WO 2019/032612, and/or WO 2020/191252, the sugar modifications and modified sugars of each of which are independently incorporated herein by reference.

In some embodiments a modified sugar is one described in U.S. Pat. Nos. 5,658,873, 5,118,800, 5,393,878, 5,514,785, 5,627,053, 7,034,133; 7,084,125, 7,399,845, 5,319,080, 5,591,722, 5,597,909, 5,466,786, 6,268,490, 6,525,191, 5,519,134, 5,576,427, 6,794,499, 6,998,484, 7,053,207, 4,981,957, 5,359,044, 6,770,748, 7,427,672, 5,446,137, 6,670,461, 7,569,686, 7,741,457, 8,022,193, 8,030,467, 8,278,425, 5,610,300, 5,646,265, 8,278,426, 5,567,811, 5,700,920, 8,278,283, 5,639,873, 5,670,633, 8,314,227, US 2008/0039618 or US 2009/0012281.

In some embodiments, a sugar modification is 2′-OMe, 2′-MOE, 2′-LNA, 2′-F, 5′-vinyl, or S-cEt. In some embodiments, a modified sugar is a sugar of FRNA, FANA, or morpholino. In some embodiments, an oligonucleotide comprises a nucleic acid analog, e.g., GNA, LNA, PNA, TNA, F-HNA (F-THP or 3′-fluoro tetrahydropyran), MNA (mannitol nucleic acid, e.g., Leumann 2002 Bioorg. Med. Chem. 10: 841-854), ANA (anitol nucleic acid), or morpholino, or a portion thereof. In some embodiments, a sugar modification replaces a natural sugar with another cyclic or acyclic moiety. Examples of such moieties are widely known in the art, e.g., those used in morpholino, glycol nucleic acids, etc. and may be utilized in accordance with the present disclosure. As appreciated by those skilled in the art, when utilized with modified sugars, in some embodiments internucleotidic linkages may be modified, e.g., as in morpholino, PNA, etc.

In some embodiments, a sugar is a 6′-modified bicyclic sugar that have either (R) or (S)-chirality at the 6-position, e.g., those described in U.S. Pat. No. 7,399,845. In some embodiments, a sugar is a 5′-modified bicyclic sugar that has either (R) or (S)-chirality at the 5-position, e.g., those described in US 20070287831.

In some embodiments, a modified sugar contains one or more substituents at the 2′ position (typically one substituent, and often at the axial position) independently selected from —F; —CF₃, —CN, —N₃, —NO, —NO₂, —OR′, —SR′, or —N(R′)₂, wherein each R′ is independently described in the present disclosure; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or—N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, a substituent is —O(CH₂), OCH₃, —O(CH₂)ₙNH₂, MOE, DMAOE, or DMAEOE, wherein wherein n is from 1 to about 10. In some embodiments, a modified sugar is one described in WO 2001/088198; and Martin et al., Helv. Chim. Acta, 1995, 78, 486-504. In some embodiments, a modified sugar comprises one or more groups selected from a substituted silyl group, an RNA cleaving group, a reporter group, a fluorescent label, an intercalator, a group for improving the pharmacokinetic properties of a nucleic acid, a group for improving the pharmacodynamic properties of a nucleic acid, or other substituents having similar properties. In some embodiments, modifications are made at one or more of the 2′, 3′, 4′, or 5′ positions, including the 3′ position of the sugar on the 3′-terminal nucleoside or in the 5′ position of the 5′-terminal nucleoside.

In some embodiments, the 2′-OH of a ribose is replaced with a group selected from —H, —F; —CF₃, —CN, —N₃, —NO, —NO₂, —OR′, —SR′, or —N(R′)₂, wherein each R′ is independently described in the present disclosure; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C1-C₁₀ alkyl)₂, —NH—(C₁-C₁₀ alkylene)-O—(C1-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe.

In some embodiments, a sugar modification is a 2′-modification. Commonly used 2′-modifications include but are not limited to 2′-OR, wherein R is not hydrogen and is as described in the present disclosure. In some embodiments, a modification is 2′-OR, wherein R is optionally substituted C₁₋₆ aliphatic. In some embodiments, a modification is 2′-OR, wherein R is optionally substituted C₁₋₆ alkyl. In some embodiments, a modification is 2′-OMe. In some embodiments, a modification is 2′-MOE. In some embodiments, a 2′-modification is S-cEt. In some embodiments, a modified sugar is an LNA sugar. In some embodiments, a 2′-modification is —F. In some embodiments, a 2′-modification is FANA. In some embodiments, a 2′-modification is FRNA. In some embodiments, a sugar modification is a 5′-modification, e.g., 5′-Me. In some embodiments, a sugar modification changes the size of the sugar ring. In some embodiments, a sugar modification is the sugar moiety in FHNA.

In some embodiments, a sugar modification replaces a sugar moiety with another cyclic or acyclic moiety. Examples of such moieties are widely known in the art, including but not limited to those used in morpholino (optionally with its phosphorodiamidate linkage), glycol nucleic acids, etc.

In some embodiments, one or more of the sugars of an oligonucleotide are modified. In some embodiments, a modified sugar comprises a 2′-modification. In some embodiments, each modified sugar independently comprises a 2′-modification. In some embodiments, a 2′-modification is 2′-OR. In some embodiments, a 2′-modification is a 2′-OMe. In some embodiments, a 2′-modification is a 2′-MOE. In some embodiments, a 2′-modification is an LNA sugar modification. In some embodiments, a 2′-modification is 2′-F. In some embodiments, each sugar modification is independently a 2′-modification. In some embodiments, each sugar modification is independently 2′-OR or 2′-F. In some embodiments, each sugar modification is independently 2′-OR or 2′-F, wherein R is optionally substituted C₁₋₆ alkyl. In some embodiments, each sugar modification is independently 2′-OR or 2′-F, wherein at least one is 2′-F. In some embodiments, each sugar modification is independently 2′-OR or 2′-F, wherein R is optionally substituted C₁₋₆ alkyl, and wherein at least one is 2′-OR. In some embodiments, each sugar modification is independently 2′-OR or 2′-F, wherein at least one is 2′-F, and at least one is 2′-OR. In some embodiments, each sugar modification is independently 2′-OR or 2′-F, wherein R is optionally substituted C₁₋₆ alkyl, and wherein at least one is 2′-F, and at least one is 2′-OR. In some embodiments, each sugar modification is independently 2′-OR. In some embodiments, each sugar modification is independently 2′-OR, wherein R is optionally substituted C₁₋₆ alkyl. In some embodiments, each sugar modification is 2′-OMe. In some embodiments, each sugar modification is 2′-MOE. In some embodiments, each sugar modification is independently 2′-OMe or 2′-MOE. In some embodiments, each sugar modification is independently 2′-OMe, 2′-MOE, or a LNA sugar.

Modified sugars include cyclobutyl or cyclopentyl moieties in place of a pentofuranosyl sugar. Representative examples of such modified sugars include those described in U.S. Pat. Nos. 4,981,957, 5,118,800, 5,319,080, or U.S. Pat. No. 5,359,044. In some embodiments, the oxygen atom within the ribose ring is replaced by nitrogen, sulfur, selenium, or carbon. In some embodiments, —O— is replaced with —N(R′)—, —S—, —Se— or —C(R′)₂—. In some embodiments, a modified sugar is a modified ribose wherein the oxygen atom within the ribose ring is replaced with nitrogen, and wherein the nitrogen is optionally substituted with an alkyl group (e.g., methyl, ethyl, isopropyl, etc.).

A non-limiting example of modified sugars is glycerol, which is part of glycerol nucleic acids (GNAs), e.g., as described in Zhang, R et al., J. Am. Chem. Soc., 2008, 130, 5846-5847; Zhang L, et al., J. Am. Chem. Soc., 2005, 127, 4174-4175 and Tsai C H et al., PNAS, 2007, 14598-14603.

A flexible nucleic acid (FNA) is based on a mixed acetal aminal of formyl glycerol, e.g., as described in Joyce G F et al., PNAS, 1987, 84, 4398-4402 and Heuberger B D and Switzer C, J. Am. Chem. Soc., 2008, 130, 412-413.

In some embodiments, an oligonucleotide, and/or a modified nucleoside thereof, comprises a sugar or modified sugar described in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the sugars and modified sugars of each of which are independently incorporated herein by reference.

In some embodiments, one or more hydroxyl group in a sugar is optionally and independently replaced with halogen, R′—N(R′)₂, —OR′, or —SR′, wherein each R′ is independently described in the present disclosure.

In some embodiments, a modified nucleoside is any modified nucleoside described in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the modified nucleosides of each of which are independently incorporated herein by reference.

In some embodiments, a sugar modification is 5′-vinyl (R or S), 5′-methyl (R or S), 2′-SH, 2′-F, 2′-OCH₃, 2′-OCH₂CH₃, 2′—OCH₂CH₂F or 2′-O(CH₂)₂₀CH₃. In some embodiments, a substituent at the 2′ position, e.g., a 2′-modification, is allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), wherein each allyl, amino and alkyl is optionally substituted, and each of R₁, Rₘ and Rₙ is independently R′ as described in the present disclosure. In some embodiments, each of Rₗ, Rₘ and Rₙ is independently —H or optionally substituted C₁-C₁₀ alkyl.

In some embodiments, bicyclic sugars comprise a bridge, e.g., -L^{b}-L^{b}-, -L-, etc. between two sugar carbons, e.g., between the 4′ and the 2′ ribosyl ring carbon atoms. In some embodiments, a bridge is 4′-(CH₂)—O-2′ (e.g., LNA sugars), 4′-(CH₂)—S-2′, 4′—(CH₂)₂—O-2′ (e.g., ENA sugars), 4′-CH(R′)—O-2′ (e.g., 4′-CH(CH₃)—O-2′, 4′-CH(CH₂OCH₃)—O-2′, and examples in U.S. Pat. No. 7,399,845, etc.), 4′-CH(R′)₂—O-2′ (e.g., 4′-C(CH₃)(CH₃)—O-2′ and examples in WO 2009006478, etc.), 4′-CH₂—N(OR′)-2′ (e.g., 4′-CH₂—N(OCH₃)-2′, examples in WO 2008150729, etc.), 4′-CH₂—O—N(R′)-2′ (e.g., 4′-CH₂—O—N(CH₃)-2′, examples in US 20040171570, etc.), 4′-CH₂—N(R′)—O-2′ [e.g., wherein R is —H, C₁-C₁₂ alkyl, or a protecting group (e.g., see U.S. Pat. No. 7,427,672)], 4′-C(R′)₂—C(H)(R′)-2′ (e.g., 4′-CH₂—C(H)(CH₃)-2′, examples in Chattopadhyaya et al., J. Org. Chem., 2009, 74, 118-134, etc.), or 4′-C(R′)₂—C(═C(R′)₂)-2′ (e.g., 4′-CH₂—C(═CH₂)-2′, examples in WO 2008154401, etc.).

In some embodiments, a sugar is a tetrahydropyran or THP sugar. In some embodiments, a modified nucleoside is tetrahydropyran nucleoside or THP nucleoside which is a nucleoside having a six-membered tetrahydropyran sugar substituted for a pentofuranosyl residue in typical natural nucleosides. THP sugars and/or nucleosides include those used in hexitol nucleic acid (HNA), anitol nucleic acid (ANA), mannitol nucleic acid (MNA) (e.g., Leumann, Bioorg. Med. Chem., 2002, 10, 841-854) or fluoro HNA (F-HNA).

In some embodiments, sugars comprise rings having more than 5 atoms and/or more than one heteroatom, e.g., morpholino sugars which are described in e.g., Braasch et al., Biochemistry, 2002, 41, 4503-4510; U.S. Pat. Nos. 5,698,685; 5,166,315; 5,185,444; 5,034,506; etc.).

As those skilled in the art will appreciate, modifications of sugars, nucleobases, internucleotidic linkages, etc. can and are often utilized in combination in oligonucleotides, e.g., see various oligonucleotides in Table 1.

In some embodiments, a nucleoside has a six-membered cyclohexenyl in place of the pentofuranosyl residue in naturally occurring nucleosides. Example cyclohexenyl nucleosides and preparation and uses thereof are described in, e.g., WO 2010036696; Robeyns et al., J. Am. Chem. Soc., 2008, 130(6), 1979-1984; Horvath et al., Tetrahedron Letters, 2007, 48, 3621-3623; Nauwelaerts et al., J. Am. Chem. Soc., 2007, 129(30), 9340-9348; Gu et al., Nucleosides, Nucleotides & Nucleic Acids, 2005, 24(5-7), 993-998; Nauwelaerts et al., Nucleic Acids Research, 2005, 33(8), 2452-2463; Robeyns et al., Acta Crystallographica, Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J. Org. Chem., 2003, 68, 4499-4505; Verbeure et al., Nucleic Acids Research, 2001, 29(24), 4941-4947; Wang et al., J. Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; WO 2006047842; WO 2001049687; etc.

Many monocyclic, bicyclic and tricyclic ring systems are suitable as sugar surrogates (modified sugars) and may be utilized in accordance with the present disclosure. See, e.g., Leumann, Christian J. Bioorg. & Med. Chem., 2002, 10, 841-854. Such ring systems can undergo various additional substitutions to further enhance their properties and/or activities.

In some embodiments, a 2′-modified sugar is a furanosyl sugar modified at the 2′ position. In some embodiments, a 2′-modification is halogen, —R′ (wherein R′ is not —H), —OR′ (wherein R′ is not —H), —SR′, —N(R′)₂, optionally substituted —CH₂—CH═CH₂, optionally substituted alkenyl, or optionally substituted alkynyl. In some embodiments, a 2′-modifications is selected from —O[(CH₂)ₙO]ₘCH₃, —O(CH₂)ₙNH₂, —O(CH₂)ₙCH₃, —O(CH₂)ₙF, —O(CH₂)ₙONH₂, —OCH₂C(═O)N(H)CH₃, and —O(CH₂), ON[(CH₂)ₙCH₃]₂, wherein each n and m is independently from 1 to about 10. In some embodiments, a 2′-modification is optionally substituted C₁-C₁₂ alkyl, optionally substituted alkenyl, optionally substituted alkynyl, optionally substituted alkaryl, optionally substituted aralkyl, optionally substituted —O-alkaryl, optionally substituted —O-aralkyl, —SH, —SCH₃, —OCN, —Cl, —Br, —CN, —F, —CF₃, —OCF₃, —SOCH₃, —SO₂CH₃, —ONO₂, —NO₂, —N₃, —NH₂, optionally substituted heterocycloalkyl, optionally substituted heterocycloalkaryl, optionally substituted aminoalkylamino, optionally substituted polyalkylamino, substituted silyl, a reporter group, an intercalator, a group for improving pharmacokinetic properties, a group for improving the pharmacodynamic properties, and other substituents. In some embodiments, a 2′-modification is a 2′-MOE modification (e.g., see Baker et al., J. Biol. Chem., 1997, 272, 11944-12000). In some cases, a 2′-MOE modification has been reported as having improved binding affinity compared to unmodified sugars and to some other modified nucleosides, such as 2′-O-methyl, 2′-O-propyl, and 2′-O-aminopropyl. Oligonucleotides having the 2′-MOE modification have also been reported to be capable of inhibiting gene expression with promising features for in vivo use (see, e.g., Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926; etc.).

In some embodiments, a 2′-modified or 2′-substituted sugar or nucleoside is a sugar or nucleoside comprising a substituent at the 2′ position of the sugar which is other than —H (typically not considered a substituent) or —OH. In some embodiments, a 2′-modified sugar is a bicyclic sugar comprising a bridge connecting two carbon atoms of the sugar ring one of which is the 2′ carbon. In some embodiments, a 2′-modification is non-bridging, e.g., allyl, amino, azido, thio, optionally substituted —O-allyl, optionally substituted —O—C₁-C₁₀ alkyl, —OCF₃, —O(CH₂)₂OCH₃, 2′—O(CH₂)₂SCH₃, —O(CH₂)₂ON(Rₘ)(Rₙ), or —OCH₂C(═O)N(Rₘ)(Rₙ), where each Rₘ and Rₙ is independently —H or optionally substituted C₁-C₁₀ alkyl.

Certain modified sugars, their preparation and uses are described in U.S. Pat. Nos. 4,981,957, 5,118,800, 5,319,080, 5,359,044, 5,393,878, 5,446,137, 5,466,786, 5,514,785, 5,519,134, 5,567,811, 5,576,427, 5,591,722, 5,597,909, 5,610,300, 5,627,053, 5,639,873, 5,646,265, 5,670,633, 5,700,920, 5,792,847, 6,600,032 and WO 2005121371.

In some embodiments, a sugar is the sugar of N-methanocarba, LNA, cMOE BNA, cEt BNA, α-L-LNA or related analogs, HNA, Me-ANA, MOE-ANA, Ara-FHNA, FHNA, R-6′-Me-FHNA, S-6′-Me-FHNA, ENA, or c-ANA. In some embodiments, a modified internucleotidic linkage is C3-amide (e.g., sugar that has the amide modification attached to the C3′, Mutisya et al. 2014 Nucleic Acids Res. 2014 Jun. 1; 42(10): 6542-6551), formacetal, thioformacetal, MMI [e.g., methylene(methylimino), Peoc′h et al. 2006 Nucleosides and Nucleotides 16 (7-9)], a PMO (phosphorodiamidate linked morpholino) linkage (which connects two sugars), or a PNA (peptide nucleic acid) linkage. In some embodiments, examples of internucleotidic linkages and/or sugars are described in Allerson et al. 2005 J. Med. Chem. 48: 901-4; BMCL 2011 21: 1122; BMCL 2011 21: 588; BMCL 2012 22: 296; Chattopadhyaya et al. 2007 J. Am. Chem. Soc. 129: 8362; Chem. Bio. Chem. 2013 14: 58; Curr. Prot. Nucl. Acids Chem. 2011 1.24.1; Egli et al. 2011 J. Am. Chem. Soc. 133: 16642; Hendrix et al. 1997 Chem. Eur. J. 3: 110; Hyrup et al. 1996 Bioorg. Med. Chem. 4: 5; Imanishi 1997 Tet. Lett. 38: 8735; J. Am. Chem. Soc. 1994, 116, 3143; J. Med. Chem. 2009 52: 10; J. Org. Chem. 2010 75: 1589; Jepsen et al. 2004 Oligo. 14: 130-146; Jones et al. J. Org. Chem. 1993, 58, 2983; Jung et al. 2014 ACIEE 53: 9893; Kodama et al. 2014 AGDS; Koizumi 2003 BMC 11: 2211; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Lima et al. 2012 Cell 150: 883-894; Mesmaeker et al. Angew. Chem., Int. Ed. Engl. 1994, 33, 226; Migawa et al. 2013 Org. Lett. 15: 4316; Mol. Ther. Nucl. Acids 2012 1: e47; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Murray et al. 2012 Nucl. Acids Res. 40: 6135; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Obika et al. 2008 J. Am. Chem. Soc. 130: 4886; Obika et al. 2011 Org. Lett. 13: 6050; Oestergaard et al. 2014 JOC 79: 8877; Pallan et al. 2012 Biochem. 51: 7; Pallan et al. 2012 Chem. Comm. 48: 8195-8197; Petersen et al. 2003 TRENDS Biotech. 21: 74-81; Prakash et al. 2010 J. Med. Chem. 53: 1636; Prakash et al. 2015 Nucl. Acids Res. 43: 2993-3011; Prakash et al. 2016 Bioorg. Med. Chem. Lett. 26: 2817-2820; Rajwanshi et al. 1999 Chem. Commun. 1395-1396; Schultz et al. 1996 Nucleic Acids Res. 24: 2966; Seth et al. 2008 Nucl. Acid Sym. Ser. 52: 553; Seth et al. 2009 J. Med. Chem. 52: 10-13; Seth et al. 2010 J. Am. Chem. Soc. 132: 14942; Seth et al. 2010 J. Med. Chem. 53: 8309-8318; Seth et al. 2010 J. Org. Chem. 75: 1569-1581; Seth et al. 2011 BMCL 21: 4690; Seth et al. 2012 Bioo. Med. Chem. Lett. 22: 296-299; Seth et al. 2012 Mol. Ther-Nuc. Acids. 1, e47; Seth et al., Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Starrup et al. 2010 Nucl. Acids Res. 38: 7100; Swayze et al. 2007 Nucl. Acids Res. 35: 687; Ts'o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; WO 2007090071; WO 2016079181; U.S. Pat. Nos. 6,326,199; 6,066,500; or U.S. Pat. No. 6,440,739.

In some embodiments, an oligonucleotide or a portion thereof (e.g., a domain, a subdomain, etc.) comprise a high level of 2′-F modified sugars, e.g., about 10%-100% (e.g., about 20%, 25%, 30%, 35%, 40%, 45%, 50%, 55%, 60%, 65%, 70%, 75%, 80%, 85%, 90%, 95%, or more, or about 100%) of sugars in an oligonucleotide or a portion thereof (e.g., a domain, a subdomain, etc.) comprises 2′-F. In some embodiments, about 50% or more of sugars in an oligonucleotide or a portion thereof comprises 2′-F. In some embodiments, about 60% or more of sugars in an oligonucleotide or a portion thereof comprises 2′-F. In some embodiments, about 70% or more of sugars in an oligonucleotide or a portion thereof comprises 2′-F. In some embodiments, about 80% or more of sugars in an oligonucleotide or a portion thereof comprises 2′-F. In some embodiments, about 90% or more of sugars in an oligonucleotide or a portion thereof comprises 2′-F. In some embodiments, an oligonucleotide or a portion thereof also comprises one or more sugars comprising no 2′-F (e.g., sugars comprising no modifications and/or sugars comprising other modifications).

In some embodiments, no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in an oligonucleotide or a portion thereof (e.g., a domain, a subdomain, etc.) comprises 2′-MOE. In some embodiments, no more than about 50% of sugars in an oligonucleotide or a portion thereof comprises 2′-MOE. In some embodiments, no sugars in an oligonucleotide or a portion thereof comprises 2′-MOE. In some embodiments, no more than 1, 2, 3, 4, or 5 sugars in an oligonucleotide or a portion thereof comprises 2′-MOE.

Various additional sugars useful for preparing oligonucleotides or analogs thereof are known in the art and may be utilized in accordance with the present disclosure.

### Internucleotidic Linkages

In some embodiments, oligonucleotides comprise base modifications, sugar modifications, and/or internucleotidic linkage modifications. Various internucleotidic linkages can be utilized in accordance with the present disclosure to link units comprising nucleobases, e.g., nucleosides. In some embodiments, provided oligonucleotides comprise both one or more modified internucleotidic linkages and one or more natural phosphate linkages. As widely known by those skilled in the art, natural phosphate linkages are widely found in natural DNA and RNA molecules; they have the structure of —OP(O)(OH)O—, connect sugars in the nucleosides in DNA and RNA, and may be in various salt forms, for example, at physiological pH (about 7.4), natural phosphate linkages are predominantly exist in salt forms with the anion being —OP(O)(O—)O—. A modified internucleotidic linkage, or a non-natural phosphate linkage, is an internucleotidic linkage that is not natural phosphate linkage or a salt form thereof. Modified internucleotidic linkages, depending on their structures, may also be in their salt forms. For example, as appreciated by those skilled in the art, phosphorothioate internucleotidic linkages which have the structure of —OP(O)(SH)O— may be in various salt forms, e.g., at physiological pH (about 7.4) with the anion being —OP(O)(S—)O—.

In some embodiments, an oligonucleotide comprises an internucleotidic linkage which is a modified internucleotidic linkage, e.g., phosphorothioate, phosphorodithioate, methylphosphonate, phosphoroamidate, thiophosphate, 3′-thiophosphate, or 5′-thiophosphate.

In some embodiments, a modified internucleotidic linkage is a chiral internucleotidic linkage which comprises a chiral linkage phosphorus. In some embodiments, a chiral internucleotidic linkage is a phosphorothioate linkage. In some embodiments, a chiral internucleotidic linkage is a non-negatively charged internucleotidic linkage. In some embodiments, a chiral internucleotidic linkage is a neutral internucleotidic linkage. In some embodiments, a chiral internucleotidic linkage is chirally controlled with respect to its chiral linkage phosphorus. In some embodiments, a chiral internucleotidic linkage is stereochemically pure with respect to its chiral linkage phosphorus. In some embodiments, a chiral internucleotidic linkage is not chirally controlled. In some embodiments, a pattern of backbone chiral centers comprises or consists of positions and linkage phosphorus configurations of chirally controlled internucleotidic linkages (Rp or Sp) and positions of achiral internucleotidic linkages (e.g., natural phosphate linkages).

In some embodiments, an internucleotidic linkage comprises a P-modification, wherein a P-modification is a modification at a linkage phosphorus. In some embodiments, a modified internucleotidic linkage is a moiety which does not comprise a phosphorus but serves to link two sugars or two moieties that each independently comprises a nucleobase, e.g., as in peptide nucleic acid (PNA).

In some embodiments, an oligonucleotide comprises a modified internucleotidic linkage, e.g., those having the structure of Formula I, I-a, I-b, or I-c and described herein and/or in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the internucleotidic linkages (e.g., those of Formula I, I-a, I-b, I-c, etc.) of each of which are independently incorporated herein by reference. In some embodiments, a modified internucleotidic linkage is a chiral internucleotidic linkage. In some embodiments, a modified internucleotidic linkage is a phosphorothioate internucleotidic linkage.

In some embodiments, a modified internucleotidic linkage is a non-negatively charged internucleotidic linkage. In some embodiments, provided oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, the present disclosure provides oligonucleotides comprising one or more neutral internucleotidic linkages. In some embodiments, a non-negatively charged internucleotidic linkage has the structure of Formula I-n-1, I-n-2, I-n-3, I-n-4, II, II-a-1, II-a-2, II-b-1, II-b-2, II-c-1, II-c-2, II-d-1, II-d-2, etc., or a salt form thereof, as described herein and/or in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, WO 2019/032612, and/or WO 2020/191252, the non-negatively charged internucleotidic linkages (e.g., those of Formula I-n-1, I-n-2, I-n-3, I-n-4, II, II-a-1, II-a-2, II-b-1, II-b-2, II-c-1, II-c-2, II-d-1, II-d-2, etc., or a suitable salt form thereof) of each of which are independently incorporated herein by reference.

In some embodiments, a non-negatively charged internucleotidic linkage can improve the delivery and/or activities (e.g., adenosine editing activity).

In some embodiments, a modified internucleotidic linkage (e.g., a non-negatively charged internucleotidic linkage) comprises optionally substituted triazolyl. In some embodiments, a modified internucleotidic linkage (e.g., a non-negatively charged internucleotidic linkage) comprises optionally substituted alkynyl. In some embodiments, a modified internucleotidic linkage comprises a triazole or alkyne moiety. In some embodiments, a triazole moiety, e.g., a triazolyl group, is optionally substituted. In some embodiments, a triazole moiety, e.g., a triazolyl group) is substituted. In some embodiments, a triazole moiety is unsubstituted. In some embodiments, a modified internucleotidic linkage comprises an optionally substituted cyclic guanidine moiety. In some embodiments, a modified internucleotidic linkage has the structure of

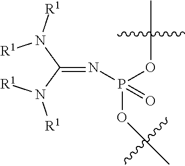

and is optionally chirally controlled, wherein R¹ is -L-R′, wherein L is L^{B} as described herein, and R′ is as described herein. In some embodiments, each R¹ is independently R′. In some embodiments, each R′ is independently R. In some embodiments, two R¹ are R and are taken together to form a ring as described herein. In some embodiments, two R¹ on two different nitrogen atoms are R and are taken together to form a ring as described herein. In some embodiments, R¹ is independently optionally substituted C₁₋₆ aliphatic as described herein. In some embodiments, R¹ is methyl. In some embodiments, two R′ on the same nitrogen atom are R and are taken together to form a ring as described herein. In some embodiments, a modified internucleotidic linkage has the structure of

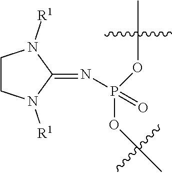

and is optionally chirally controlled. In some embodiments, is

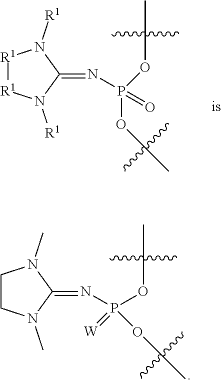

In some embodiments, a modified internucleotidic linkage comprises an optionally substituted cyclic guanidine moiety and has the structure of:

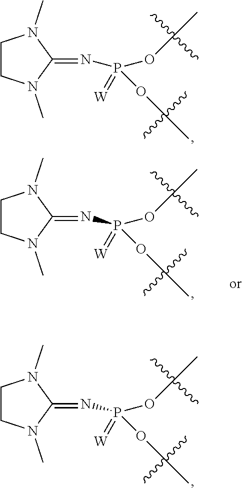

or wherein W is O or S. In some embodiments, W is O. In some embodiments, W is S. In some embodiments, a non-negatively charged internucleotidic linkage is stereochemically controlled.

In some embodiments, a non-negatively charged internucleotidic linkage or a neutral internucleotidic linkage is an internucleotidic linkage comprising a triazole moiety. In some embodiments, a non-negatively charged internucleotidic linkage or a non-negatively charged internucleotidic linkage comprises an optionally substituted triazolyl group. In some embodiments, an internucleotidic linkage comprising a triazole moiety (e.g., an optionally substituted triazolyl group) has the structure of

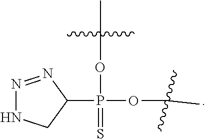 [SMILES: *OP(=S)(O*)C1CNN=N1]

In some embodiments, an internucleotidic linkage comprising a triazole moiety has the structure of

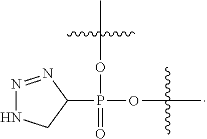 [SMILES: *OP(=O)(O*)C1CNN=N1]

In some embodiments, an internucleotidic linkage comprising a triazole moiety has the formula of

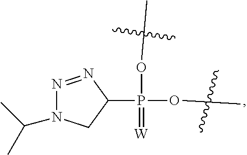

where W is O or S. In some embodiments, an internucleotidic linkage comprising an alkyne moiety (e.g., an optionally substituted alkynyl group) has the formula of

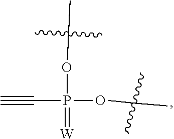

wherein W is O or S. In some embodiments, an internucleotidic linkage, e.g., a non-negatively charged internucleotidic linkage, a neutral internucleotidic linkage, comprises a cyclic guanidine moiety. In some embodiments, an internucleotidic linkage comprising a cyclic guanidine moiety has the structure of

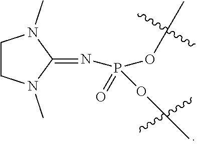 [SMILES: *OP(=O)(N=C1N(C)CCN1C)O*]

In some embodiments, a non-negatively charged internucleotidic linkage, or a neutral internucleotidic linkage, is or comprising a structure selected from

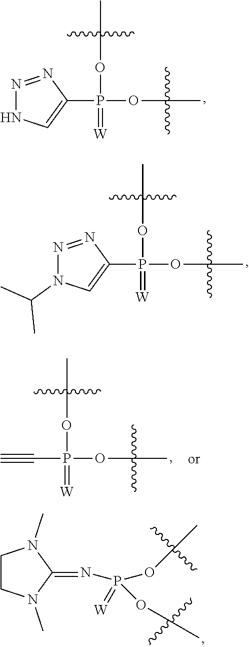

wherein W is O or S.

In some embodiments, an internucleotidic linkage comprises a Tmg group

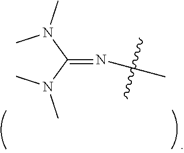

In some embodiments, an internucleotidic linkage comprises a Tmg group and has the structure of

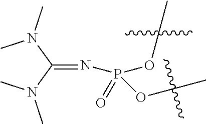 [SMILES: *OP(=O)(N=C(N(C)C)N(C)C)O*]

(the “Tmg internucleotidic linkage”). In some embodiments, neutral internucleotidic linkages include internucleotidic linkages of PNA and PMO, and an Tmg internucleotidic linkage.

In some embodiments, a non-negatively charged internucleotidic linkage has the structure of Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, etc., or a salt form thereof. In some embodiments, a non-negatively charged internucleotidic linkage comprises an optionally substituted 3-20 membered heterocyclyl or heteroaryl group having 1-10 heteroatoms. In some embodiments, a non-negatively charged internucleotidic linkage comprises an optionally substituted 3-20 membered heterocyclyl or heteroaryl group having 1-10 heteroatoms, wherein at least one heteroatom is nitrogen. In some embodiments, such a heterocyclyl or heteroaryl group is of a 5-membered ring. In some embodiments, such a heterocyclyl or heteroaryl group is of a 6-membered ring.

In some embodiments, a non-negatively charged internucleotidic linkage comprises an optionally substituted 5-20 membered heteroaryl group having 1-10 heteroatoms. In some embodiments, a non-negatively charged internucleotidic linkage comprises an optionally substituted 5-20 membered heteroaryl group having 1-10 heteroatoms, wherein at least one heteroatom is nitrogen. In some embodiments, a non-negatively charged internucleotidic linkage comprises an optionally substituted 5-6 membered heteroaryl group having 1-4 heteroatoms, wherein at least one heteroatom is nitrogen. In some embodiments, a non-negatively charged internucleotidic linkage comprises an optionally substituted 5-membered heteroaryl group having 1-4 heteroatoms, wherein at least one heteroatom is nitrogen. In some embodiments, a heteroaryl group is directly bonded to a linkage phosphorus. In some embodiments, a non-negatively charged internucleotidic linkage comprises an optionally substituted triazolyl group. In some embodiments, a non-negatively charged internucleotidic linkage comprises an unsubstituted triazolyl group, e.g.,

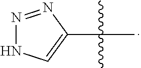 [SMILES: *c1c[nH]nn1]

In some embodiments, a non-negatively charged internucleotidic linkage comprises a substituted triazolyl group, e.g.,

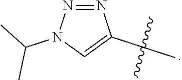 [SMILES: *c1cn(C(C)C)nn1]

In some embodiments, a non-negatively charged internucleotidic linkage comprises an optionally substituted 5-20 membered heterocyclyl group having 1-10 heteroatoms. In some embodiments, a non-negatively charged internucleotidic linkage comprises an optionally substituted 5-20 membered heterocyclyl group having 1-10 heteroatoms, wherein at least one heteroatom is nitrogen. In some embodiments, a non-negatively charged internucleotidic linkage comprises an optionally substituted 5-6 membered heterocyclyl group having 1-4 heteroatoms, wherein at least one heteroatom is nitrogen. In some embodiments, a non-negatively charged internucleotidic linkage comprises an optionally substituted 5-membered heterocyclyl group having 1-4 heteroatoms, wherein at least one heteroatom is nitrogen. In some embodiments, at least two heteroatoms are nitrogen. In some embodiments, a heterocyclyl group is directly bonded to a linkage phosphorus. In some embodiments, a heterocyclyl group is bonded to a linkage phosphorus through a linker, e.g., ═N— when the heterocyclyl group is part of a guanidine moiety who directed bonded to a linkage phosphorus through its ═N—. In some embodiments, a non-negatively charged internucleotidic linkage comprises an optionally substituted

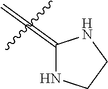 [SMILES: *=C1NCCN1]

group. In some embodiments, a non-negatively charged internucleotidic linkage comprises an substituted

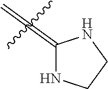 [SMILES: *=C1NCCN1]

group. In some embodiments, a non-negatively charged internucleotidic linkage comprises a

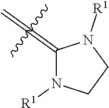

group, wherein each R¹ is independently -L-R. In some embodiments, each R¹ is independently optionally substituted C₁₋₆ alkyl. In some embodiments, each R¹ is independently methyl.

In some embodiments, a modified internucleotidic linkage, e.g., a non-negatively charged internucleotidic linkage, comprises a triazole or alkyne moiety, each of which is optionally substituted. In some embodiments, a modified internucleotidic linkage comprises a triazole moiety. In some embodiments, a modified internucleotidic linkage comprises a unsubstituted triazole moiety. In some embodiments, a modified internucleotidic linkage comprises a substituted triazole moiety. In some embodiments, a modified internucleotidic linkage comprises an alkyl moiety. In some embodiments, a modified internucleotidic linkage comprises an optionally substituted alkynyl group. In some embodiments, a modified internucleotidic linkage comprises an unsubstituted alkynyl group. In some embodiments, a modified internucleotidic linkage comprises a substituted alkynyl group. In some embodiments, an alkynyl group is directly bonded to a linkage phosphorus.

In some embodiments, an oligonucleotide comprises different types of internucleotidic phosphorus linkages. In some embodiments, a chirally controlled oligonucleotide comprises at least one natural phosphate linkage and at least one modified (non-natural) internucleotidic linkage. In some embodiments, an oligonucleotide comprises at least one natural phosphate linkage and at least one phosphorothioate. In some embodiments, an oligonucleotide comprises at least one non-negatively charged internucleotidic linkage. In some embodiments, an oligonucleotide comprises at least one natural phosphate linkage and at least one non-negatively charged internucleotidic linkage. In some embodiments, an oligonucleotide comprises at least one phosphorothioate internucleotidic linkage and at least one non-negatively charged internucleotidic linkage. In some embodiments, an oligonucleotide comprises at least one phosphorothioate internucleotidic linkage, at least one natural phosphate linkage, and at least one non-negatively charged internucleotidic linkage. In some embodiments, oligonucleotides comprise one or more, e.g., 1-50, 1-40, 1-30, 1-20, 1-15, 1-10, 1, 2, 3, 4, 5, 6, 7, 8, 9, 10, 11, 12, 13, 14, 15, 16, 17, 18, 19, 20 or more non-negatively charged internucleotidic linkages. In some embodiments, a non-negatively charged internucleotidic linkage is not negatively charged in that at a given pH in an aqueous solution less than 50%, 40%, 40%, 30%, 20%, 10%, 5%, or 1% of the internucleotidic linkage exists in a negatively charged salt form. In some embodiments, a pH is about pH 7.4. In some embodiments, a pH is about 4-9. In some embodiments, the percentage is less than 10%. In some embodiments, the percentage is less than 5%. In some embodiments, the percentage is less than 1%. In some embodiments, an internucleotidic linkage is a non-negatively charged internucleotidic linkage in that the neutral form of the internucleotidic linkage has no pKa that is no more than about 1, 2, 3, 4, 5, 6, or 7 in water. In some embodiments, no pKa is 7 or less. In some embodiments, no pKa is 6 or less. In some embodiments, no pKa is 5 or less. In some embodiments, no pKa is 4 or less. In some embodiments, no pKa is 3 or less. In some embodiments, no pKa is 2 or less. In some embodiments, no pKa is 1 or less. In some embodiments, pKa of the neutral form of an internucleotidic linkage can be represented by pKa of the neutral form of a compound having the structure of CH₃-the internucleotidic linkage-CH₃. For example, pKa of the neutral form of an internucleotidic linkage having the structure of Formula I may be represented by the pKa of the neutral form of a compound having the structure of

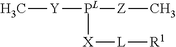

(wherein each of X, Y, Z is independently —O—, —S—, —N(R′)—; L is L^{B}, and R¹ is -L-R′), pKa of

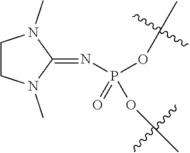 [SMILES: *OP(=O)(N=C1N(C)CCN1C)O*]

can be represented by pKa

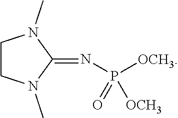

In some embodiments, a non-negatively charged internucleotidic linkage is a neutral internucleotidic linkage. In some embodiments, a non-negatively charged internucleotidic linkage is a positively-charged internucleotidic linkage. In some embodiments, a non-negatively charged internucleotidic linkage comprises a guanidine moiety. In some embodiments, a non-negatively charged internucleotidic linkage comprises a heteroaryl base moiety. In some embodiments, a non-negatively charged internucleotidic linkage comprises a triazole moiety. In some embodiments, a non-negatively charged internucleotidic linkage comprises an alkynyl moiety.

In some embodiments, a neutral or non-negatively charged internucleotidic linkage has the structure of any neutral or non-negatively charged internucleotidic linkage described in any of: U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, WO 2019/032612, and/or WO 2020/191252,2607, WO2019032612, WO 2019/055951, WO 2019/075357, WO 2019/200185, WO 2019/217784, WO 2019/032612, and/or WO 2020/191252, each neutral or non-negatively charged internucleotidic linkage of each of which is hereby incorporated by reference.

In some embodiments, each R′ is independently optionally substituted C₁₋₆ aliphatic. In some embodiments, each R′ is independently optionally substituted C₁₋₆ alkyl. In some embodiments, each R′ is independently —CH₃. In some embodiments, each R^{s} is —H.

In some embodiments, a non-negatively charged internucleotidic linkage has the structure of

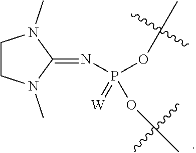

In some embodiments, a non-negatively charged internucleotidic linkage has the structure of

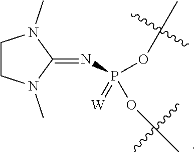

In some embodiments, a non-negatively charged internucleotidic linkage has the structure of

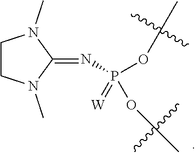

In some embodiments, a non-negatively charged internucleotidic linkage has the structure of

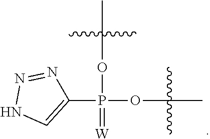

In some embodiments, a non-negatively charged internucleotidic linkage has the structure of

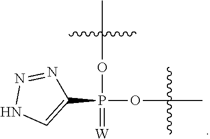

In some embodiments, a non-negatively charged internucleotidic linkage has the structure of

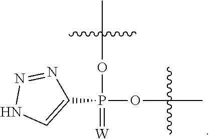

In some embodiments, a non-negatively charged internucleotidic linkage has the structure of

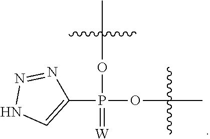

In some embodiments, a non-negatively charged internucleotidic linkage has the structure of

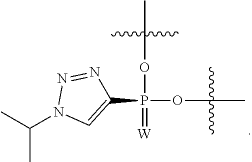

In some embodiments, a non-negatively charged internucleotidic linkage has the structure of

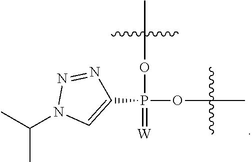

In some embodiments, a non-negatively charged internucleotidic linkage has the structure of

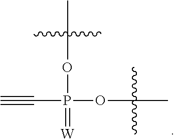

In some embodiments, a non-negatively charged internucleotidic linkage has the structure of

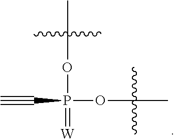

In some embodiments, a non-negatively charged internucleotidic linkage has the structure of

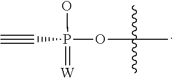

In some embodiments, W is O. In some embodiments, W is S. In some embodiments, a neutral internucleotidic linkage is a non-negatively charged internucleotidic linkage described above.

In some embodiments, provided oligonucleotides comprise 1 or more internucleotidic linkages of Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2, which are described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, WO 2019/032607, WO2019032612, WO 2019/055951, WO 2019/075357, WO 2019/200185, WO 2019/217784, WO 2019/032612, and/or WO 2020/191252, the Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2, or salt forms thereof, each of which are independently incorporated herein by reference.

In some embodiments, an oligonucleotide comprises a neutral internucleotidic linkage and a chirally controlled internucleotidic linkage. In some embodiments, an oligonucleotide comprises a neutral internucleotidic linkage and a chirally controlled internucleotidic linkage which is not the neutral internucleotidic linkage. In some embodiments, an oligonucleotide comprises a neutral internucleotidic linkage and a chirally controlled phosphorothioate internucleotidic linkage. In some embodiments, the present disclosure provides an oligonucleotide comprising one or more non-negatively charged internucleotidic linkages and one or more phosphorothioate internucleotidic linkages, wherein each phosphorothioate internucleotidic linkage in the oligonucleotide is independently a chirally controlled internucleotidic linkage. In some embodiments, the present disclosure provides an oligonucleotide comprising one or more neutral internucleotidic linkages and one or more phosphorothioate internucleotidic linkage, wherein each phosphorothioate internucleotidic linkage in the oligonucleotide is independently a chirally controlled internucleotidic linkage. In some embodiments, an oligonucleotide comprises at least 5, 6, 7, 8, 9, 10, 11, 12, 13, 14, 15, 16, 17, 18, 19, 20 or more chirally controlled phosphorothioate internucleotidic linkages. In some embodiments, non-negatively charged internucleotidic linkage is chirally controlled. In some embodiments, non-negatively charged internucleotidic linkage is not chirally controlled. In some embodiments, a neutral internucleotidic linkage is chirally controlled. In some embodiments, a neutral internucleotidic linkage is not chirally controlled.

Without wishing to be bound by any particular theory, the present disclosure notes that a neutral internucleotidic linkage can be more hydrophobic than a phosphorothioate internucleotidic linkage (PS), which can be more hydrophobic than a natural phosphate linkage (PO). Typically, unlike a PS or PO, a neutral internucleotidic linkage bears less charge. Without wishing to be bound by any particular theory, the present disclosure notes that incorporation of one or more neutral internucleotidic linkages into an oligonucleotide may increase oligonucleotides' ability to be taken up by a cell and/or to escape from endosomes. Without wishing to be bound by any particular theory, the present disclosure notes that incorporation of one or more neutral internucleotidic linkages can be utilized to modulate melting temperature of duplexes formed between an oligonucleotide and its target nucleic acid.

Without wishing to be bound by any particular theory, the present disclosure notes that incorporation of one or more non-negatively charged internucleotidic linkages, e.g., neutral internucleotidic linkages, into an oligonucleotide may be able to increase the oligonucleotide's ability to mediate a function such as target adenosine editing.

As appreciated by those skilled in the art, internucleotidic linkages such as natural phosphate linkages and those of Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, or salt forms thereof typically connect two nucleosides (which can either be natural or modified) as described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, WO 2019/032612, and/or WO 2020/191252, the Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, or salt forms thereof, each of which are independently incorporated herein by reference. A typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms or heteroatoms (e.g., Y and Z in various formulae) with one optionally modified ribose or deoxyribose at its 5′ carbon, and the other optionally modified ribose or deoxyribose at its 3′ carbon. In some embodiments, each nucleoside units connected by an internucleotidic linkage independently comprises a nucleobase which is independently an optionally substituted A, T, C, G, or U, or a substituted tautomer of A, T, C, G or U, or a nucleobase comprising an optionally substituted heterocyclyl and/or a heteroaryl ring having at least one nitrogen atom.

As appreciated by those skilled in the art, many other types of internucleotidic linkages may be utilized in accordance with the present disclosure, for example, those described in U.S. Pat. Nos. 3,687,808; 4,469,863; 4,476,301; 5,177,195; 5,023,243; 5,034,506; 5,166,315; 5,185,444; 5,188,897; 5,214,134; 5,216,141; 5,235,033; 5,264,423; 5,264,564; 5,276,019; 5,278,302; 5,286,717; 5,321,131; 5,399,676; 5,405,938; 5,405,939; 5,434,257; 5,453,496; 5,455,233; 5,466,677; 5,466,677; 5,470,967; 5,476,925; 5,489,677; 5,519,126; 5,536,821; 5,541,307; 5,541,316; 5,550,111; 5,561,225; 5,563,253; 5,571,799; 5,587,361; 5,596,086; 5,602,240; 5,608,046; 5,610,289; 5,618,704; 5,623,070; 5,625,050; 5,633,360; 5,64,562; 5,663,312; 5,677,437; 5,677,439; 6,160,109; 6,239,265; 6,028,188; 6,124,445; 6,169,170; 6,172,209; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; or RE39464. In some embodiments, a modified internucleotidic linkage is one described in U.S. Pat. No. 9,982,257, US 20170037399, US 20180216108, WO 2017192664, WO 2017015575, WO2017062862, WO 2018067973, WO 2017160741, WO 2017192679, WO 2017210647, WO 2018098264, PCT/US18/35687, PCT/US18/38835, or PCT/US18/51398, the nucleobases, sugars, internucleotidic linkages, chiral auxiliaries/reagents, and technologies for oligonucleotide synthesis (reagents, conditions, cycles, etc.) of each of which is independently incorporated herein by reference.

In some embodiments, each internucleotidic linkage in an oligonucleotide is independently selected from a natural phosphate linkage, a phosphorothioate linkage, and a non-negatively charged internucleotidic linkage (e.g., n001). In some embodiments, each internucleotidic linkage in an oligonucleotide is independently selected from a natural phosphate linkage, a phosphorothioate linkage, and a neutral internucleotidic linkage (e.g., n001).

In some embodiments, an oligonucleotide comprises one or more nucleotides that independently comprise a phosphorus modification prone to “autorelease” under certain conditions. That is, under certain conditions, a particular phosphorus modification is designed such that it self-cleaves from the oligonucleotide to provide, e.g., a natural phosphate linkage. In some embodiments, such a phosphorus modification has a structure of —O-L-R¹, wherein L is L^{B} as described herein, and R¹ is R′ as described herein. In some embodiments, a phosphorus modification has a structure of —S-L-R¹, wherein each L and R¹ is independently as described in the present disclosure. Certain examples of such phosphorus modification groups can be found in U.S. Pat. No. 9,982,257. In some embodiments, an autorelease group comprises a morpholino group. In some embodiments, an autorelease group is characterized by the ability to deliver an agent to the internucleotidic phosphorus linker, which agent facilitates further modification of the phosphorus atom such as, e.g., desulfurization. In some embodiments, the agent is water and the further modification is hydrolysis to form a natural phosphate linkage.

In some embodiments, an oligonucleotide comprises one or more internucleotidic linkages that improve one or more pharmaceutical properties and/or activities of the oligonucleotide. It is well documented in the art that certain oligonucleotides are rapidly degraded by nucleases and exhibit poor cellular uptake through the cytoplasmic cell membrane (Poijarvi-Virta et al., Curr. Med. Chem. (2006), 13(28); 3441-65; Wagner et al., Med. Res. Rev. (2000), 20(6):417-51; Peyrottes et al., Mini Rev. Med. Chem. (2004), 4(4):395-408; Gosselin et al., (1996), 43(1):196-208; Bologna et al., (2002), Antisense & Nucleic Acid Drug Development 12:33-41). Vives et al. (Nucleic Acids Research (1999), 27(20):4071-76) reported that tert-butyl SATE pro-oligonucleotides displayed markedly increased cellular penetration compared to the parent oligonucleotide under certain conditions.

Oligonucleotides can comprise various number of natural phosphate linkages. In some embodiments, 5% or more of the internucleotidic linkages of provided oligonucleotides are natural phosphate linkages. In some embodiments, 10% or more of the internucleotidic linkages of provided oligonucleotides are natural phosphate linkages. In some embodiments, 15% or more of the internucleotidic linkages of provided oligonucleotides are natural phosphate linkages. In some embodiments, 20% or more of the internucleotidic linkages of provided oligonucleotides are natural phosphate linkages. In some embodiments, 25% or more of the internucleotidic linkages of provided oligonucleotides are natural phosphate linkages. In some embodiments, 30% or more of the internucleotidic linkages of provided oligonucleotides are natural phosphate linkages. In some embodiments, 35% or more of the internucleotidic linkages of provided oligonucleotides are natural phosphate linkages. In some embodiments, 40% or more of the internucleotidic linkages of provided oligonucleotides are natural phosphate linkages. In some embodiments, provided oligonucleotides comprises 1, 2, 3, 4, 5, 6, 7, 8, 9, 10 or more natural phosphate linkages. In some embodiments, provided oligonucleotides comprises 4, 5, 6, 7, 8, 9, 10 or more natural phosphate linkages. In some embodiments, the number of natural phosphate linkages is 2. In some embodiments, the number of natural phosphate linkages is 3. In some embodiments, the number of natural phosphate linkages is 4. In some embodiments, the number of natural phosphate linkages is 5. In some embodiments, the number of natural phosphate linkages is 6. In some embodiments, the number of natural phosphate linkages is 7. In some embodiments, the number of natural phosphate linkages is 8. In some embodiments, some or all of the natural phosphate linkages are consecutive.

In some embodiments, the present disclosure demonstrates that, in at least some cases, Sp internucleotidic linkages, among other things, at the 5′- and/or 3′-end can improve oligonucleotide stability. In some embodiments, the present disclosure demonstrates that, among other things, natural phosphate linkages and/or Rp internucleotidic linkages may improve removal of oligonucleotides from a system. As appreciated by a person having ordinary skill in the art, various assays known in the art can be utilized to assess such properties in accordance with the present disclosure.

In some embodiments, each phosphorothioate internucleotidic linkage in an oligonucleotide or a portion thereof (e.g., a domain, a subdomain, etc.) is independently chirally controlled. In some embodiments, each is independently Sp or Rp. In some embodiments, a high level is Sp as described herein. In some embodiments, each phosphorothioate internucleotidic linkage in an oligonucleotide or a portion thereof is chirally controlled and is Sp. In some embodiments, one or more, e.g., about 1-5 (e.g., about 1, 2, 3, 4, or 5) is Rp.

In some embodiments, as illustrated in certain examples, an oligonucleotide or a portion thereof comprises one or more non-negatively charged internucleotidic linkages, each of which is optionally and independently chirally controlled. In some embodiments, each non-negatively charged internucleotidic linkage is independently n001. In some embodiments, a chiral non-negatively charged internucleotidic linkage is not chirally controlled. In some embodiments, each chiral non-negatively charged internucleotidic linkage is not chirally controlled. In some embodiments, a chiral non-negatively charged internucleotidic linkage is chirally controlled. In some embodiments, a chiral non-negatively charged internucleotidic linkage is chirally controlled and is Rp. In some embodiments, a chiral non-negatively charged internucleotidic linkage is chirally controlled and is Sp. In some embodiments, each chiral non-negatively charged internucleotidic linkage is chirally controlled. In some embodiments, the number of non-negatively charged internucleotidic linkages in an oligonucleotide or a portion thereof is about 1-10, or about 1, 2, 3, 4, 5, 6, 7, 8, 9, or 10. In some embodiments, it is about 1. In some embodiments, it is about 2. In some embodiments, it is about 3. In some embodiments, it is about 4. In some embodiments, it is about 5. In some embodiments, it is about 6. In some embodiments, it is about 7. In some embodiments, it is about 8. In some embodiments, it is about 9. In some embodiments, it is about 10. In some embodiments, two or more non-negatively charged internucleotidic linkages are consecutive. In some embodiments, no two non-negatively charged internucleotidic linkages are consecutive. In some embodiments, all non-negatively charged internucleotidic linkages in an oligonucleotide or a portion thereof are consecutive (e.g., 3 consecutive non-negatively charged internucleotidic linkages). In some embodiments, a non-negatively charged internucleotidic linkage, or two or more (e.g., about 2, about 3, about 4 etc.) consecutive non-negatively charged internucleotidic linkages, are at the 3′-end of an oligonucleotide or a portion thereof. In some embodiments, the last two or three or four internucleotidic linkages of an oligonucleotide or a portion thereof comprise at least one internucleotidic linkage that is not a non-negatively charged internucleotidic linkage. In some embodiments, the last two or three or four internucleotidic linkages of an oligonucleotide or a portion thereof comprise at least one internucleotidic linkage that is not n001. In some embodiments, the internucleotidic linkage linking the first two nucleosides of an oligonucleotide or a portion thereof is a non-negatively charged internucleotidic linkage. In some embodiments, the internucleotidic linkage linking the last two nucleosides of an oligonucleotide or a portion thereof is a non-negatively charged internucleotidic linkage. In some embodiments, the internucleotidic linkage linking the first two nucleosides of an oligonucleotide or a portion thereof is a phosphorothioate internucleotidic linkage. In some embodiments, it is Sp. In some embodiments, the internucleotidic linkage linking the last two nucleosides of an oligonucleotide or a portion thereof is a phosphorothioate internucleotidic linkage. In some embodiments, it is Sp.

In some embodiments, one or more chiral internucleotidic linkages are chirally controlled and one or more chiral internucleotidic linkages are not chirally controlled. In some embodiments, each phosphorothioate internucleotidic linkage is independently chirally controlled, and one or more non-negatively charged internucleotidic linkage are not chirally controlled. In some embodiments, each phosphorothioate internucleotidic linkage is independently chirally controlled, and each non-negatively charged internucleotidic linkage is not chirally controlled. In some embodiments, the internucleotidic linkage between the first two nucleosides of an oligonucleotide is a non-negatively charged internucleotidic linkage. In some embodiments, the internucleotidic linkage between the last two nucleosides are each independently a non-negatively charged internucleotidic linkage. In some embodiments, both are independently non-negatively charged internucleotidic linkages. In some embodiments, an oligonucleotide comprises one or more additional internucleotidic linkages, e.g., one of which is between the nucleosides at positions −1 and −2 relative to an nucleoside opposite to a target nucleoside (e.g., a target adenosine) (the two nucleosides immediately 3′ to an nucleoside opposite to a target nucleoside (e.g., in . . . N₀N₋₁N₋₂ . . . , N₀ is an nucleoside opposite to a target nucleoside, N₋₁ and N₋₂ are at positions −1 and −2, respectively). In some embodiments, each non-negatively charged internucleotidic linkage is independently neutral internucleotidic linkage. In some embodiments, each non-negatively charged internucleotidic linkage is independently n001.

As demonstrated herein, in some embodiments, non-negatively charged internucleotidic linkages such as n001 may provide improved properties and/or activities. In some embodiments, in an oligonucleotide a 5′-end internucleotidic linkage and/or a 3′-end internucleotidic linkage, each of which is independently bonded to two nucleosides comprising a nucleobase as described herein, is a non-negatively charged internucleotidic linkage as described herein. In some embodiments, the first one or more (e.g., the first 1, 2, and/or 3), and/or the last one or more (e.g., the last 1, 2, 3, 4, 5, 6 or 7) internucleotidic linkages, each of which is independently bonded to two nucleosides in a first domain, is independently a non-negatively charged internucleotidic linkage. In some embodiments, the first internucleotidic linkage of a first domain is a non-negatively charged internucleotidic linkage. In some embodiments, the last internucleotidic linkage that bonds to two nucleosides of a first domain is a non-negatively charged internucleotidic linkage. In some embodiments, the last internucleotidic linkage of a second domain is a non-negatively charged internucleotidic linkage. In some embodiments, one or more of internucleotidic linkages in the middle of a second domain, e.g., one or more of the 4^{th}, 5^{th} and 6^{th} internucleotidic linkages, each of which independently bonds to two nucleosides of a second domain, is independently a non-negatively charged internucleotidic linkage. In some embodiments, the 11^{th} internucleotidic linkage that bonds to two nucleosides of a second domain is a non-negatively charged internucleotidic linkage. In some embodiments, an internucleotidic linkage that is not bonded to a nucleoside opposite to a target nucleoside but is bonded to its 3′ immediate nucleoside is a non-negatively charged internucleotidic linkage. In some embodiments, a non-negatively charged internucleotidic linkage is a neutral internucleotidic linkage. In some embodiments, a non-negatively charged internucleotidic linkage is n001. In some embodiments, each non-negatively charged internucleotidic linkage is n001. In some embodiments, a non-negatively charged internucleotidic linkage is stereorandom. In some embodiments, a non-negatively charged internucleotidic linkage is chirally controlled and is Rp. In some embodiments, a non-negatively charged internucleotidic linkage is chirally controlled and is Sp. In some embodiments, each non-negatively charged internucleotidic linkage is independently chirally controlled. In some embodiments, one or more internucleotidic linkages of a first domain, e.g., one or more of the 4^{th}, 5^{th}, 6^{th}, 7^{th} and 8^{th} internucleotidic linkages each of which is independently bonded to two nucleosides of a first domain, is independently not a non-negatively charged internucleotidic linkage. In some embodiments, one or more internucleotidic linkages of a second domain, e.g., one or more of the 1^{st}, 2^{nd}, 3^{rd}, 7^{th}, 8^{th}, 9^{th}, 12^{th} and 13^{th} internucleotidic linkages each of which is independently bonded to two nucleosides of a first domain, is independently not a non-negatively charged internucleotidic linkage. In some embodiments, one or both of the 2^{nd} and the 3^{rd} internucleotidic linkages of a second domain is not a non-negatively charged internucleotidic linkage. In some embodiments, an internucleotidic linkage that is not a non-negatively charged internucleotidic linkage is a phosphorothioate internucleotidic linkage. In some embodiments, it is a stereorandom phosphorothioate internucleotidic linkage. In some embodiments, it is a Rp chirally controlled phosphorothioate internucleotidic linkage. In some embodiments, it is a Sp chirally controlled phosphorothioate internucleotidic linkage.

In some embodiments, a controlled level of oligonucleotides in a composition are desired oligonucleotides. In some embodiments, of all oligonucleotides in a composition that share a common base sequence (e.g., a desired sequence for a purpose), or of all oligonucleotides in a composition, level of desired oligonucleotides (which may exist in various forms (e.g., salt forms) and typically differ only at non-chirally controlled internucleotidic linkages (various forms of the same stereoisomer can be considered the same for this purpose)) is about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%. In some embodiments, a level is at least about 50%. In some embodiments, a level is at least about 60%. In some embodiments, a level is at least about 70%. In some embodiments, a level is at least about 75%. In some embodiments, a level is at least about 80%. In some embodiments, a level is at least about 85%. In some embodiments, a level is at least about 90%. In some embodiments, a level is or is at least (DS)^{nc}, wherein DS is about 90%, 91%, 92%, 93%, 94%, 95%, 96%, 97%, 98%, 99% or 99.5% and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, a level is or is at least (DS)^{nc}, wherein DS is 95%-100%.

Various types of internucleotidic linkages may be utilized in combination of other structural elements, e.g., sugars, to achieve desired oligonucleotide properties and/or activities. For example, the present disclosure routinely utilizes modified internucleotidic linkages and modified sugars, optionally with natural phosphate linkages and natural sugars, in designing oligonucleotides. In some embodiments, the present disclosure provides an oligonucleotide comprising one or more modified sugars. In some embodiments, the present disclosure provides an oligonucleotide comprising one or more modified sugars and one or more modified internucleotidic linkages, one or more of which are natural phosphate linkages.

In some embodiments, provided oligonucleotides comprise a number of natural RNA sugars (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more, two or more or all of them are optionally consecutive). In some embodiments, such oligonucleotides comprise modified sugars, e.g., 2′-OR modified sugars wherein R is not —H (e.g., 2-OMe, 2-MOE, etc.) at one or both ends, and/or various modified internucleotidic linkages (e.g., phosphorothioate internucleotidic linkages, non-negatively charged internucleotidic linkages, etc.). In some embodiments, at the 5′-end there are one or more, e.g., 1, 2, 3, 4, 5, 6, 7, 8, 9, or 10 more such 2′-OR modified sugars, wherein R is not —H. In some embodiments, at the 3′-end there are one or more, e.g., 1, 2, 3, 4, 5, 6, 7, 8, 9, or 10 more such 2′-OR modified sugars, wherein R is not —H. In some embodiments, each 2′-modified sugar is independently a 2′-OR modified sugar wherein R is not —H. In some embodiments, as described herein, 2′-OR is 2′-OMe. In some embodiments, 2′-OR is 2′-MOE. In some embodiments, each of 2′-OR is independently 2′-OMe or 2′-MOE. In some embodiments, each 2′-OR is 2′-OMe.

### Additional Chemical Moieties

In some embodiments, an oligonucleotide comprises one or more additional chemical moieties. Various additional chemical moieties, e.g., targeting moieties, carbohydrate moieties, lipid moieties, etc. are known in the art and can be utilized in accordance with the present disclosure to modulate properties and/or activities of provided oligonucleotides, e.g., stability, half life, activities, delivery, pharmacodynamics properties, pharmacokinetic properties, etc. In some embodiments, certain additional chemical moieties facilitate delivery of oligonucleotides to desired cells, tissues and/or organs, including but not limited the cells of the central nervous system. In some embodiments, certain additional chemical moieties facilitate internalization of oligonucleotides. In some embodiments, certain additional chemical moieties increase oligonucleotide stability. In some embodiments, the present disclosure provides technologies for incorporating various additional chemical moieties into oligonucleotides.

In some embodiments, an oligonucleotide comprises an additional chemical moiety demonstrates increased delivery to and/or activity in an tissue compared to a reference oligonucleotide, e.g., a reference oligonucleotide which does not have the additional chemical moiety but is otherwise identical.

In some embodiments, non-limiting examples of additional chemical moieties include carbohydrate moieties, targeting moieties, etc., which, when incorporated into oligonucleotides, can improve one or more properties. In some embodiments, an additional chemical moiety is selected from: glucose, GluNAc (N-acetyl amine glucosamine) and anisamide moieties. In some embodiments, a provided oligonucleotide can comprise two or more additional chemical moieties, wherein the additional chemical moieties are identical or non-identical, or are of the same category (e.g., carbohydrate moiety, sugar moiety, targeting moiety, etc.) or not of the same category.

In some embodiments, an additional chemical moiety is a targeting moiety. In some embodiments, an additional chemical moiety is or comprises a carbohydrate moiety. In some embodiments, an additional chemical moiety is or comprises a lipid moiety. In some embodiments, an additional chemical moiety is or comprises a ligand moiety for, e.g., cell receptors such as a sigma receptor, an asialoglycoprotein receptor, etc. In some embodiments, a ligand moiety is or comprises an anisamide moiety, which may be a ligand moiety for a sigma receptor. In some embodiments, a ligand moiety is or comprises a GalNAc moiety, which may be a ligand moiety for an asialoglycoprotein receptor. In some embodiments, an additional chemical moiety facilitates delivery to liver.

In some embodiments, a provided oligonucleotide can comprise one or more linkers and additional chemical moieties (e.g., targeting moieties), and/or can be chirally controlled or not chirally controlled, and/or have a bases sequence and/or one or more modifications and/or formats as described herein.

Various linkers, carbohydrate moieties and targeting moieties, including many known in the art, can be utilized in accordance with the present disclosure. In some embodiments, a carbohydrate moiety is a targeting moiety. In some embodiments, a targeting moiety is a carbohydrate moiety.

In some embodiments, a provided oligonucleotide comprises an additional chemical moiety suitable for delivery, e.g., glucose, GluNAc (N-acetyl amine glucosamine), anisamide, or a structure selected from:

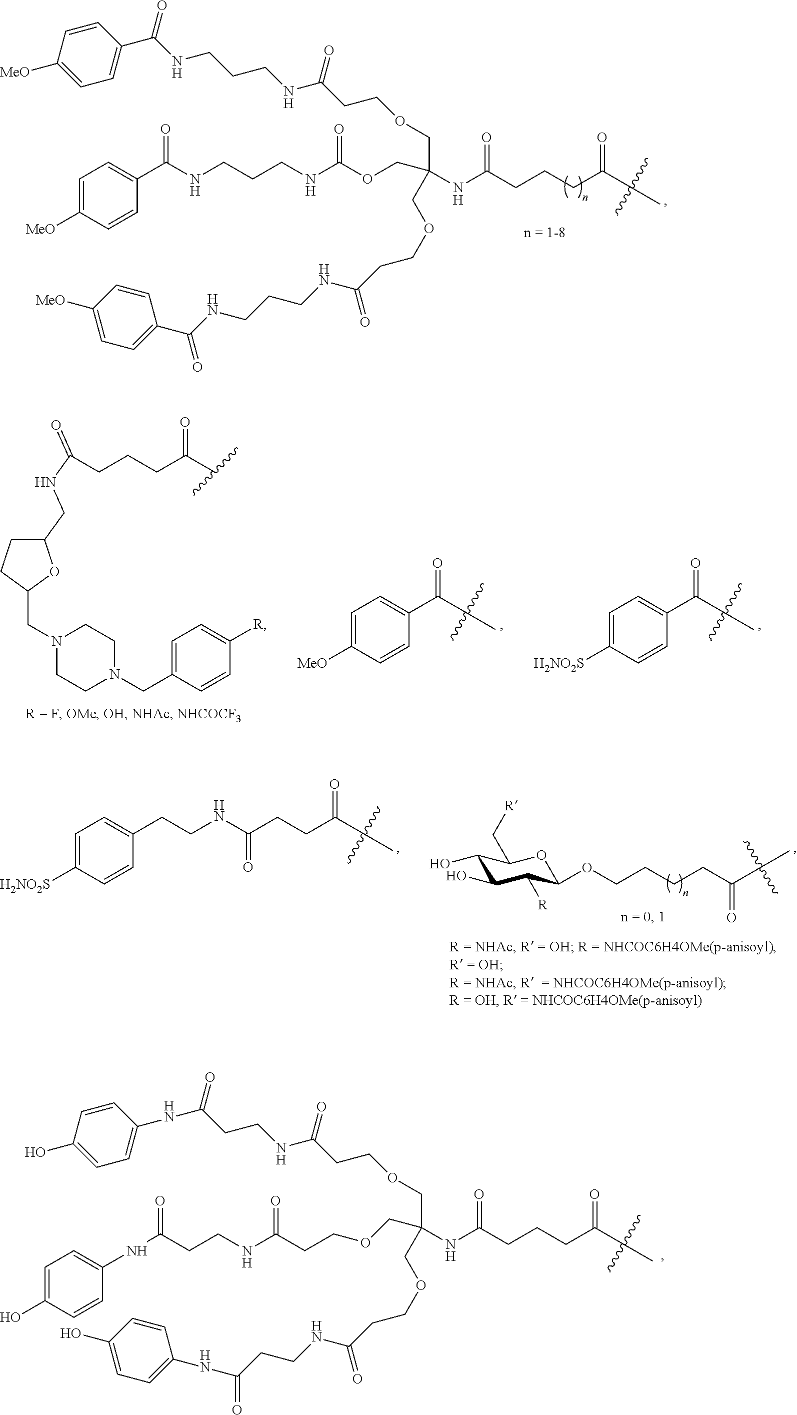

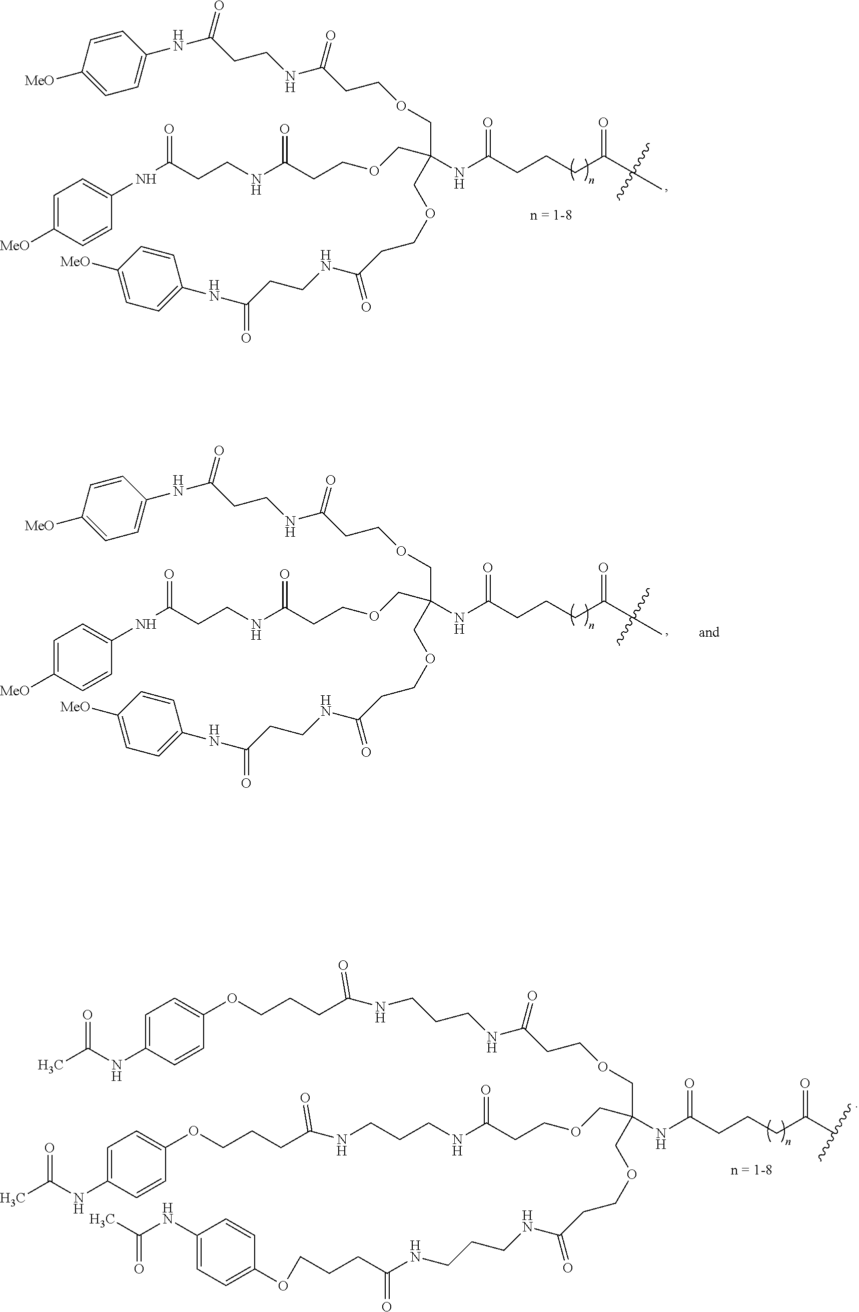

In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8.

In some embodiments, additional chemical moieties are any of ones described in the Examples, including examples of various additional chemical moieties incorporated into various oligonucleotides.

In some embodiments, an additional chemical moiety conjugated to an oligonucleotide is capable of targeting the oligonucleotide to a cell in the central nervous system.

In some embodiments, an additional chemical moiety comprises or is a cell receptor ligand. In some embodiments, an additional chemical moiety comprises or is a protein binder, e.g., one binds to a cell surface protein. Such moieties among other things can be useful for targeted delivery of oligonucleotides to cells expressing the corresponding receptors or proteins. In some embodiments, an additional chemical moiety of a provided oligonucleotide comprises anisamide or a derivative or an analog thereof and is capable of targeting the oligonucleotide to a cell expressing a particular receptor, such as the sigma 1 receptor.

In some embodiments, a provided oligonucleotide is formulated for administration to a body cell and/or tissue expressing its target. In some embodiments, an additional chemical moiety conjugated to an oligonucleotide is capable of targeting the oligonucleotide to a cell.

In some embodiments, an additional chemical moiety is selected from optionally substituted phenyl,

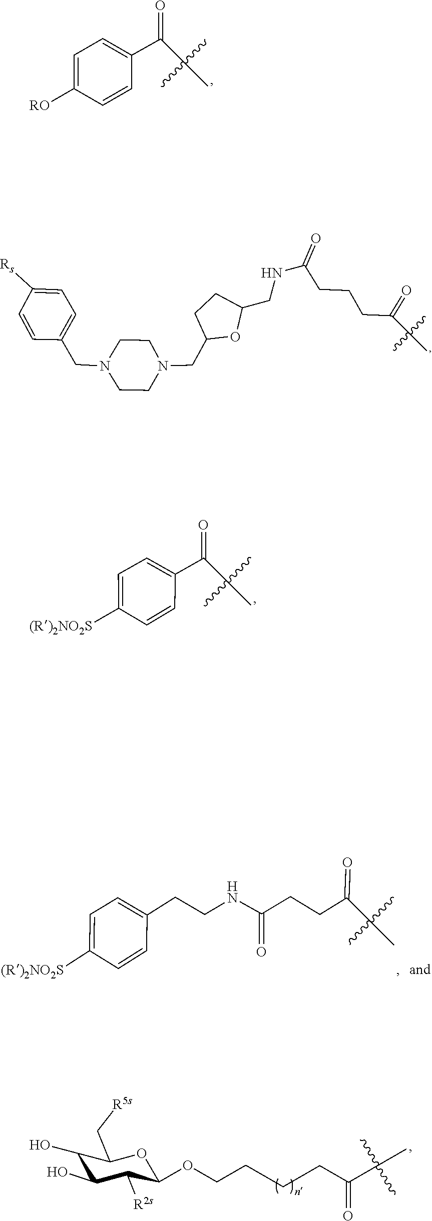

wherein n′ is 1, 2, 3, 4, 5, 6, 7, 8, 9, or 10, and each other variable is as described in the present disclosure. In some embodiments, R^{s} is F. In some embodiments, R^{s} is OMe. In some embodiments, R^{s} is OH. In some embodiments, R^{s} is NHAc. In some embodiments, R^{s} is NHCOCF₃. In some embodiments, R′ is H. In some embodiments, R is H. In some embodiments, R^{2s} is NHAc, and R^{5s} is OH. In some embodiments, R^{2s} is p-anisoyl, and R^{5s} is OH. In some embodiments, R^{2s} is NHAc and R^{5s} is p-anisoyl. In some embodiments, R^{2s} is OH, and R^{5s} is p-anisoyl. In some embodiments, an additional chemical moiety is selected from

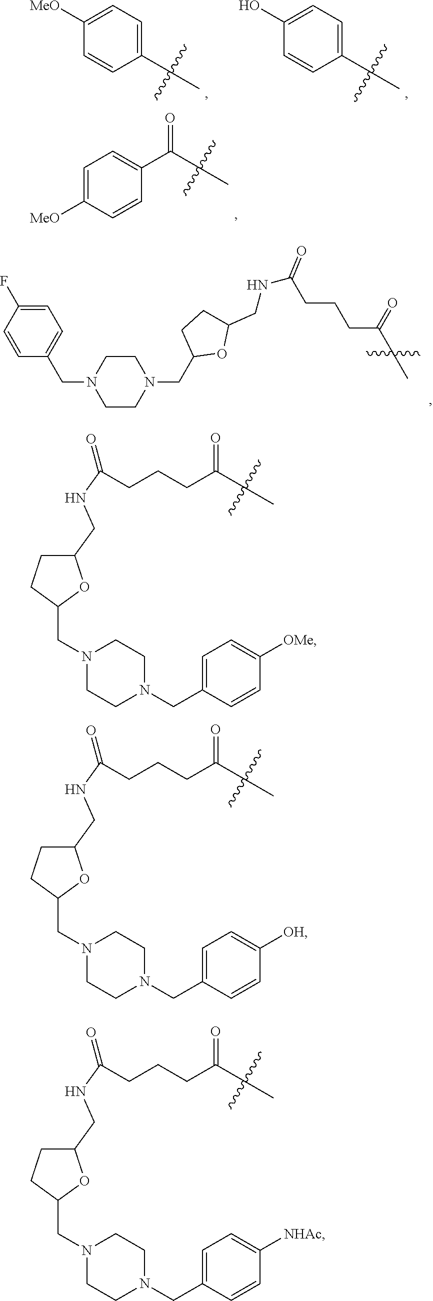

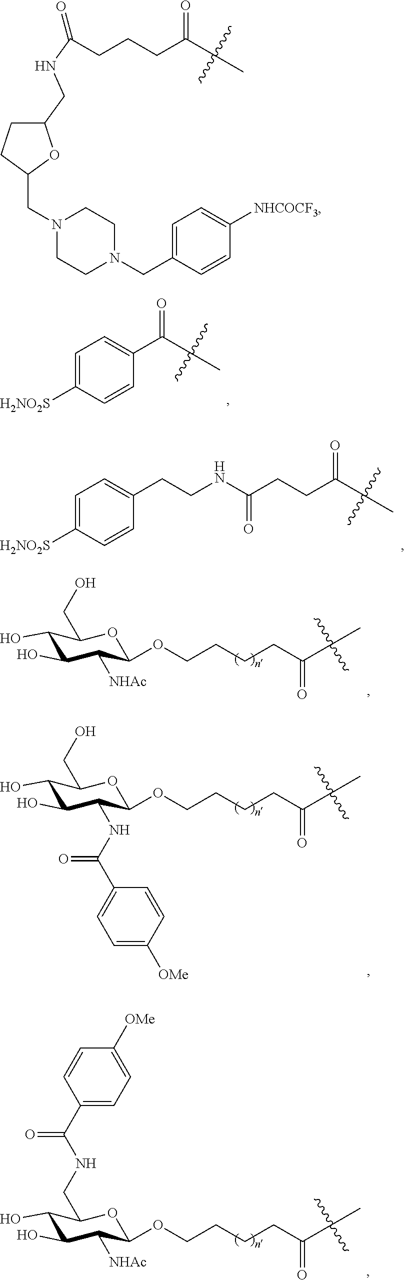

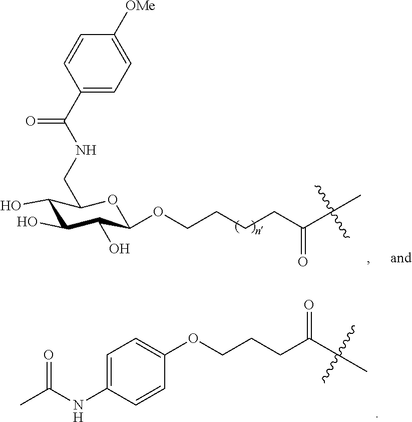

In some embodiments, n′ is 1. In some embodiments, n′ is 0. In some embodiments, n″ is 1. In some embodiments, n″ is 2.

In some embodiments, an additional chemical moiety is or comprises an asialoglycoprotein receptor (ASGPR) ligand.

Without wishing to be bound by any particular theory, the present disclosure notes that ASGPR1 has also been reported to be expressed in the hippocampus region and/or cerebellum Purkinje cell layer of the mouse. http://mouse.brain-map.org/experiment/show/2048

Various other ASGPR ligands are known in the art and can be utilized in accordance with the present disclosure. In some embodiments, an ASGPR ligand is a carbohydrate. In some embodiments, an ASGPR ligand is GalNac or a derivative or an analog thereof. In some embodiments, an ASGPR ligand is one described in Sanhueza et al. J. Am. Chem. Soc., 2017, 139 (9), pp 3528-3536. In some embodiments, an ASGPR ligand is one described in Mamidyala et al. J. Am. Chem. Soc., 2012, 134, pp 1978-1981. In some embodiments, an ASGPR ligand is one described in US 20160207953. In some embodiments, an ASGPR ligand is a substituted-6,8-dioxabicyclo[3.2.1]octane-2,3-diol derivative disclosed in, e.g., US 20160207953. In some embodiments, an ASGPR ligand is one described in, e.g., US 20150329555. In some embodiments, an ASGPR ligand is a substituted-6,8-dioxabicyclo[3.2.1]octane-2,3-diol derivative disclosed e.g., in US 20150329555. In some embodiments, an ASGPR ligand is one described in U.S. Pat. No. 8,877,917, US 20160376585, U.S. Ser. No. 10/086,081, or U.S. Pat. No. 8,106,022. ASGPR ligands described in these documents are incorporated herein by reference. Those skilled in the art will appreciate that various technologies are known in the art, including those described in these documents, for assessing binding of a chemical moiety to ASGPR and can be utilized in accordance with the present disclosure. In some embodiments, a provided oligonucleotide is conjugated to an ASGPR ligand. In some embodiments, a provided oligonucleotide comprises an ASGPR ligand. In some embodiments, an additional chemical moiety comprises an ASGPR ligand is

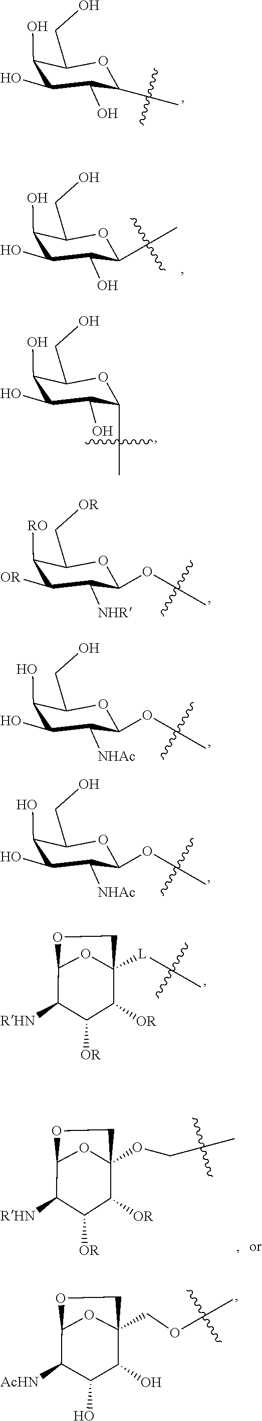

wherein each variable is independently as described in the present disclosure. In some embodiments, R is —H. In some embodiments, R′ is —C(O)R.

In some embodiments, an additional chemical moiety is or comprises

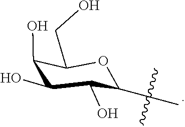

In some embodiments, an additional chemical moiety is or comprises

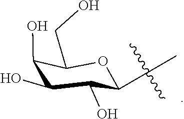

In some embodiments, an additional chemical moiety is or comprises

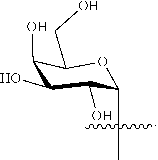

In some embodiments, an additional chemical moiety is or comprises

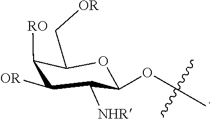

In some embodiments, an additional chemical moiety is or comprises optionally substituted

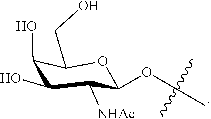

In some embodiments, an additional chemical moiety is or comprises

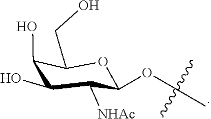

In some embodiments, an additional chemical moiety is or comprises

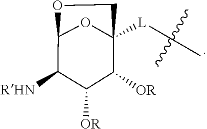

In some embodiments, an additional chemical moiety is or comprises

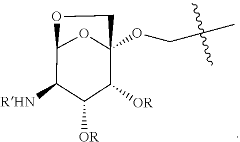

In some embodiments, an additional chemical moiety is or comprises

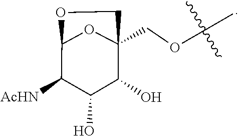 [SMILES: *OC[C@@]12CO[C@@H](O1)[C@H](NC(C)=O)[C@@H](O)[C@H]2O]

In some embodiments, an additional chemical moiety comprises one or more moieties that can bind to, e.g., oligonucleotide target cells. For example, in some embodiments, an additional chemistry moiety comprises one or more protein ligand moieties, e.g., in some embodiments, an additional chemical moiety comprises multiple moieties, each of which independently is an ASGPR ligand. In some embodiments, as in Mod 001 and Mod083, an additional chemical moiety comprises three such ligands. Mod001:

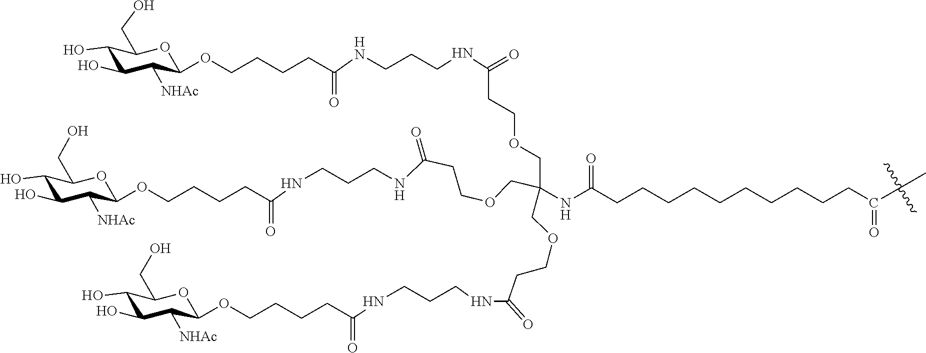

### Mod083:

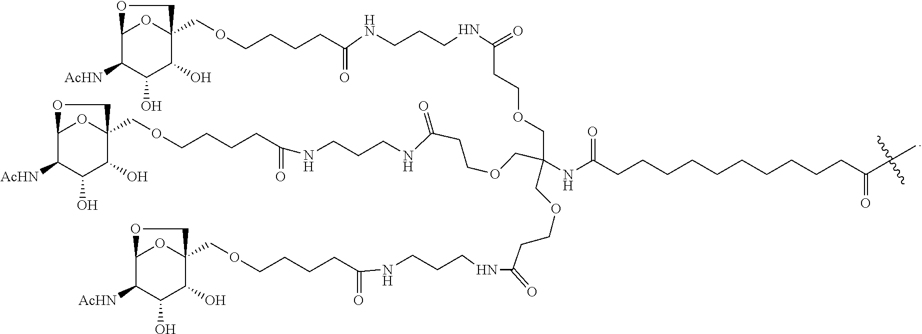 [SMILES: *C(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O]

In some embodiments, an oligonucleotide comprises

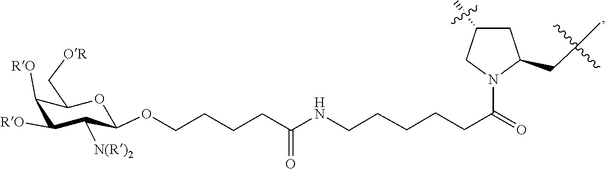

wherein each variable is independently as described herein. In some embodiments, each —OR′ is —OAc, and —N(R′)₂ is —NHAc. In some embodiments, an oligonucleotide comprises

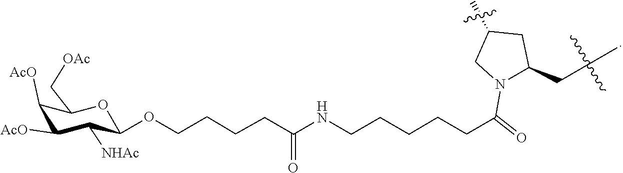

In some embodiments, each R′ is —H. In some embodiments, each —OR′ is —OH, and each —N(R′)₂ is —NHC(O)R. In some embodiments, each —OR′ is —OH, and each —N(R′)₂ is —NHAc. In some embodiments, an oligonucleotide comprises

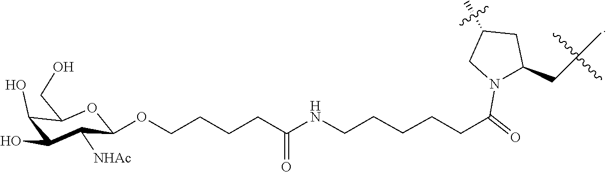

In some embodiments, the —CH₂— connection site is utilized as a C5 connection site in a sugar. In some embodiments, the connection site on the ring is utilized as a C3 connection site in a sugar. Such moieties may be introduced utilizing, e.g., phosphoramidites such as

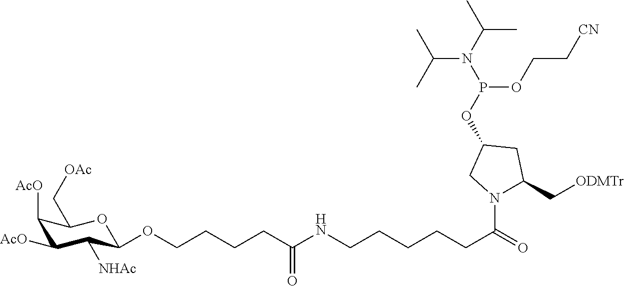

(those skilled in the art appreciate that one or more other groups, such as protection groups for —OH, —NH₂—, —N(i-Pr)₂, —OCH₂CH₂CN, etc., may be alternatively utilized, and protection groups can be removed under various suitable conditions, sometimes during oligonucleotide de-protection and/or cleavage steps). In some embodiments, an oligonucleotide comprises 2, 3 or more (e.g., 3 and no more than 3)

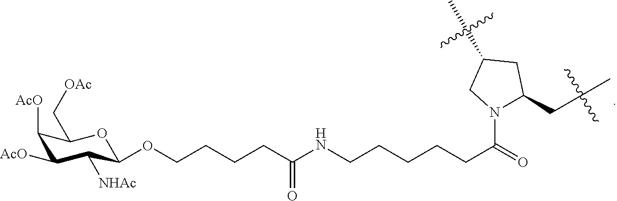

In some embodiments, an oligonucleotide comprises 2, 3 or more (e.g., 3 and no more than 3)

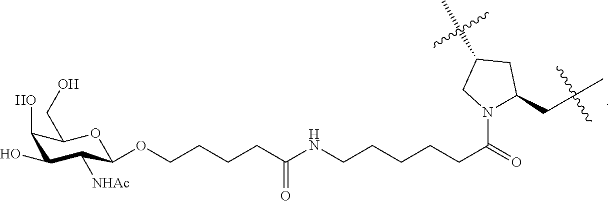

In some embodiments, copies of such moieties are linked by internucleotidic linkages, e.g., natural phosphate linkages, as described herein. In some embodiments, when at a 5′-end, a —CH₂— connection site is bonded to —OH. In some embodiments, an oligonucleotide comprises

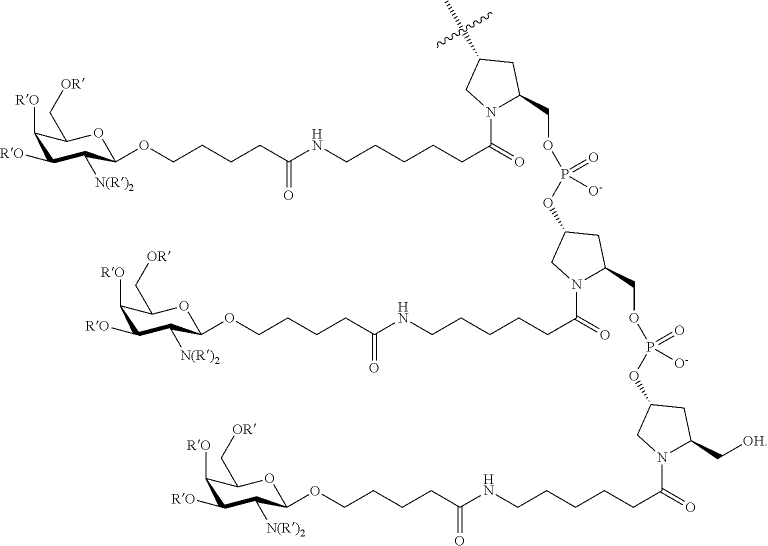

In some embodiments, each —OR′ is —OAc, and —N(R′)₂ is —NHAc. In some embodiments, an oligonucleotide comprises

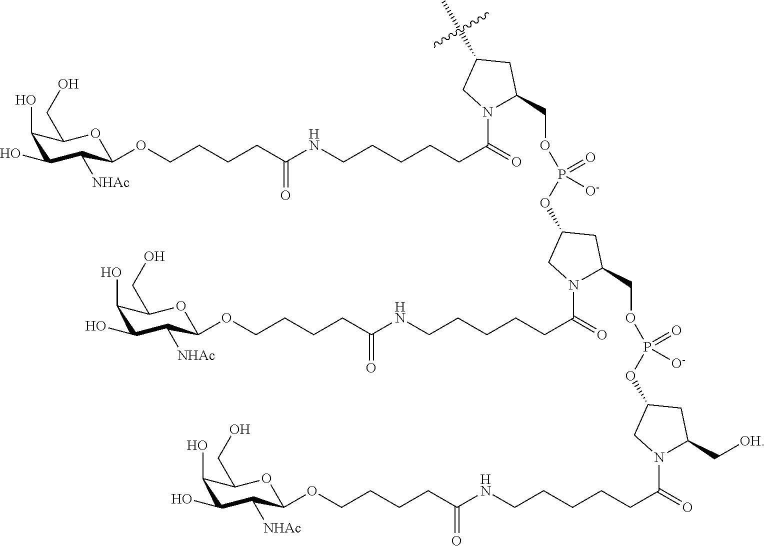

Among other things,

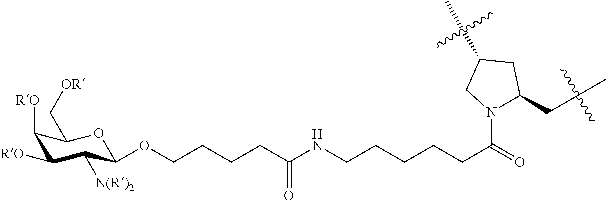

may be utilized to introduce

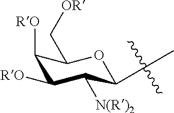

with comparable and/or better activities and/or properties. In some embodiments, it provides improved preparation efficiency and/or lower cost for the same number of

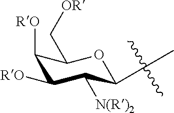

(e.g., when compared to Mod001).

In some embodiments, an additional chemical moiety is a Mod group described herein, e.g., in Table 1.

In some embodiments, an additional chemical moiety is Mod001. In some embodiments, an additional chemical moiety is Mod083. In some embodiments, an additional chemical moiety, e.g., a Mod group, is directly conjugated (e.g., without a linker) to the remainder of the oligonucleotide. In some embodiments, an additional chemical moiety is conjugated via a linker to the remainder of the oligonucleotide. In some embodiments, additional chemical moieties, e.g., Mod groups, may be directly connected, and/or via a linker, to nucleobases, sugars and/or internucleotidic linkages of oligonucleotides. In some embodiments, Mod groups are connected, either directly or via a linker, to sugars. In some embodiments, Mod groups are connected, either directly or via a linker, to 5′-end sugars. In some embodiments, Mod groups are connected, either directly or via a linker, to 5′-end sugars via 5′ carbon. For examples, see various oligonucleotides in Table 1. In some embodiments, Mod groups are connected, either directly or via a linker, to 3′-end sugars. In some embodiments, Mod groups are connected, either directly or via a linker, to 3′-end sugars via 3′ carbon. In some embodiments, Mod groups are connected, either directly or via a linker, to nucleobases. In some embodiments, Mod groups are connected, either directly or via a linker, to internucleotidic linkages. In some embodiments, provided oligonucleotides comprise Mod001 connected to 5′-end of oligonucleotide chains through L001.

As appreciated by those skilled in the art, an additional chemical moiety may be connected to an oligonucleotide chain at various locations, e.g., 5′-end, 3′-end, or a location in the middle (e.g., on a sugar, a base, an internucleotidic linkage, etc.). In some embodiments, it is connected at a 5′-end. In some embodiments, it is connected at a 3′-end. In some embodiments, it is connected at a nucleotide in the middle.

Certain additional chemical moieties (e.g., lipid moieties, targeting moieties, carbohydrate moieties), including but not limited to Mod012, Mod039, Mod062, Mod085, Mod086, and Mod094, and various linkers for connecting additional chemical moieties to oligonucleotide chains, including but not limited to L001, L003, L004, L008, L009, and L010, are described in WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, WO 2019/032612, and/or WO 2020/191252, the additional chemical moieties and linkers of each of which are independently incorporated herein by reference, and can be utilized in accordance with the present disclosure. In some embodiments, an additional chemical moiety is digoxigenin or biotin or a derivative thereof.

In some embodiments, an oligonucleotide comprises a linker, e.g., L001 L004, L008, and/or an additional chemical moiety, e.g., Mod012, Mod039, Mod062, Mod085, Mod086, or Mod094. In some embodiments, a linker, e.g., L001, L003, L004, L008, L009, L110, etc. is linked to a Mod, e.g., Mod012, Mod039, Mod062, Mod085, Mod086, Mod094, etc.

L001: —NH—(CH₂)₆— linker (also known as a C6 linker, C6 amine linker or C6 amino linker), connected to Mod, if any, through —NH—, and the 5′-end or 3′-end of the oligonucleotide chain through either a phosphate linkage (—O—P(O)(OH)—O—, which may exist as a salt form, and may be indicated as O or PO) or a phosphorothioate linkage (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration) as indicated at the —CH₂— connecting site. If no Mod is present, L001 is connected to —H through —NH—;

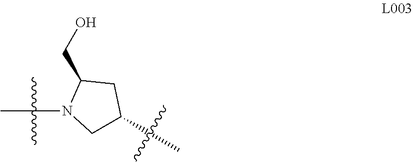 [SMILES: *[C@H]1C[C@H](CO)N(*)C1]

linker. In some embodiments, it is connected to Mod, if any (if no Mod, —H), through its amino group, and the 5′-end or 3′-end of an oligonucleotide chain e.g., via a linkage (e.g., a phosphate linkage (O or PO) or a phosphorothioate linkage (can be either not chirally controlled or chirally controlled (Sp or Rp)));
L004: linker having the structure of —NH(CH₂)₄CH(CH₂OH)CH₂—, wherein —NH— is connected to Mod (through —C(O)—) or —H, and the —CH₂— connecting site is connected to an oligonucleotide chain (e.g., at the 3′-end) through a linkage, e.g., phosphodiester (—O—P(O)(OH)—O—, which may exist as a salt form, and may be indicated as O or PO), phosphorothioate (—O—P(O)(SH)—O—, which may exist as a salt form, and may be indicated as *if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)—O—, which may exist as a salt form, and may be indicated as PS2 or: or D) linkage. For example, an asterisk immediately preceding a L004 (e.g., *L004) indicates that the linkage is a phosphorothioate linkage, and the absence of an asterisk immediately preceding L004 indicates that the linkage is a phosphodiester linkage. For example, in an oligonucleotide which terminates in . . . mAL004, the linker L004 is connected (via the —CH₂— site) through a phosphodiester linkage to the 3′ position of the 3′-terminal sugar (which is 2′-OMe modified and connected to the nucleobase A), and the L004 linker is connected via —NH— to —H. Similarly, in one or more oligonucleotides, the L004 linker is connected (via the —CH₂— site) through the phosphodiester linkage to the 3′ position of the 3′-terminal sugar, and the L004 is connected via —NH— to, e.g., Mod012, Mod085, Mod086, etc.;
L008: linker having the structure of —C(O)—(CH₂)₉—, wherein —C(O)— is connected to Mod (through —NH—) or —OH (if no Mod indicated), and the —CH₂— connecting site is connected to an oligonucleotide chain (e.g., at the 5′-end) through a linkage, e.g., phosphodiester (—O—P(O)(OH)—O—, which may exist as a salt form, and may be indicated as O or PO), phosphorothioate (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)—O—, which may exist as a salt form, and may be indicated as PS2 or: or D) linkage. For example, in an example oligonucleotide which has the sequence of 5′-L008 mN*mN*mN*mN*N*N*N*N*N*N*N*N*N*N*mN*mN*mN*mN-3′, and which has a Stereochemistry/Linkage of OXXXXXXXXX XXXXXXXX, wherein N is a base, wherein O is a natural phosphate internucleotidic linkage, and wherein X is a stereorandom phosphorothioate, L008 is connected to —OH through —C(O)—, and the 5′-end of an oligonucleotide chain through a phosphate linkage (indicated as “O” in “Stereochemistry/Linkage”); in another example oligonucleotide, which has the sequence of 5′-Mod062L008 mN*mN*mN*mN*N*N*N*N*N*N*N*N*N*N*mN*mN*mN*mN-3′, and which has a Stereochemistry/Linkage of OXXXXXXXXX XXXXXXXX, wherein N is a base, L008 is connected to Mod062 through —C(O)—, and the 5′-end of an oligonucleotide chain through a phosphate linkage (indicated as “O” in “Stereochemistry/Linkage”);
L009: —CH₂CH₂CH₂—. In some embodiments, when L009 is present at the 5′-end of an oligonucleotide without a Mod, one end of L009 is connected to —OH and the other end connected to a 5′-carbon of the oligonucleotide chain e.g., via a linkage (e.g., a phosphate linkage (O or PO) or a phosphorothioate linkage (can be either not chirally controlled or chirally controlled (Sp or Rp)));

### L010:

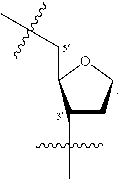

In some embodiments, when L010 is present at the 5′-end of an oligonucleotide without a Mod, the 5′-carbon of L010 is connected to —OH and the 3′-carbon connected to a 5′-carbon of the oligonucleotide chain e.g., via a linkage (e.g., a phosphate linkage (O or PO) or a phosphorothioate linkage (can be either not chirally controlled or chirally controlled (Sp or Rp)));
Mod012 (in some embodiments, —C(O)— connects to —NH— of a linker such as L001, L004, L008, etc.):

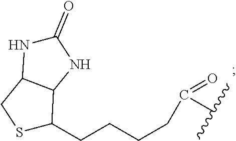 [SMILES: *C(=O)CCCCC1SCC2NC(=O)NC21]

Mod039 (in some embodiments, —C(O)— connects to —NH— of a linker such as L001, L003, L004, L008, L009, L110, etc.):

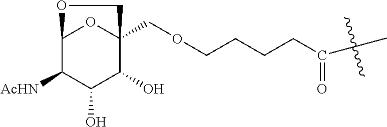 [SMILES: *C(=O)CCCCOC[C@@]12CO[C@@H](O1)[C@H](NC(C)=O)[C@@H](O)[C@H]2O]

Mod062 (in some embodiments, —C(O)— connects to —NH— of a linker such as L001, L003, L004, L008, L009, L110, etc.):

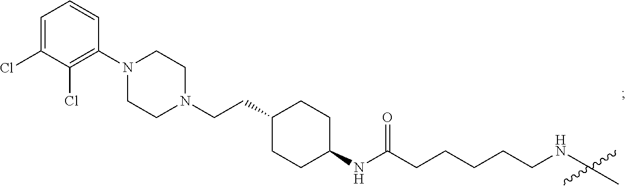 [SMILES: *NCCCCCC(=O)N[C@H]1CC[C@H](CCN2CCN(c3cccc(Cl)c3Cl)CC2)CC1]

Mod085 (in some embodiments, —C(O)— connects to —NH— of a linker such as L001, L003, L004, L008, L009, L110, etc.):

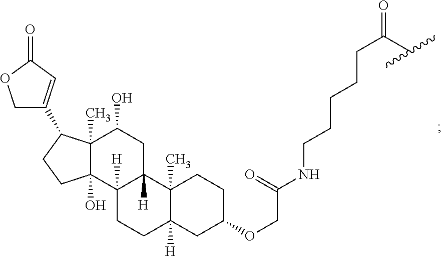

Mod086 (in some embodiments, —C(O)— connects to —NH— of a linker such as L001, L003, L004, L008, L009, L110, etc.):

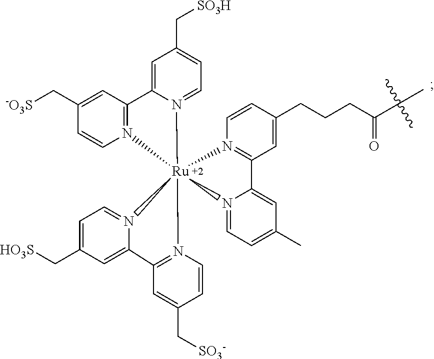

Mod094 (in some embodiments, connects to an internucleotidic linkage, or to the 5′-end or 3′-end of an oligonucleotide via a linkage, e.g., a phosphate linkage, a phosphorothioate linkage (which is optionally chirally controlled), etc. For example, in an example oligonucleotide which has the sequence of 5′-mN*mN*mN*mN*N*N*N*N*N*N*N*N*N*N*mN*mN*mN*mNMod094-3′, andwhich has a Stereochemistry/Linkage of XXXXX XXXXX XXXXX XXO, wherein N is a base, Mod094 is connected to the 3′-end of the oligonucleotide chain (3′-carbon of the 3′-end sugar) through a phosphate group (which is not shown below and which may exist as a salt form; and which is indicated as “0” in “Stereochemistry/Linkage” ( . . . XXXXO))):

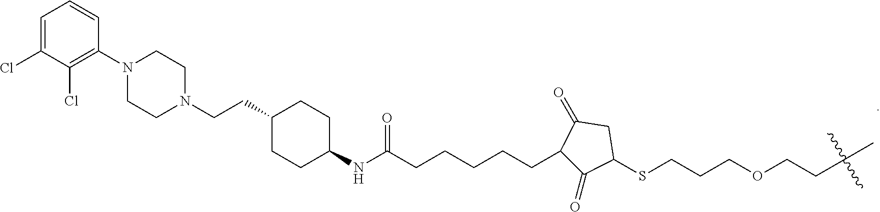 [SMILES: *CCOCCCSC1CC(=O)C(CCCCCC(=O)N[C@H]2CC[C@H](CCN3CCN(c4cccc(Cl)c4Cl)CC3)CC2)C1=O]

In some embodiments, an additional chemical moiety is one described in WO 2012/030683. In some embodiments, a provided oligonucleotide comprise a chemical structure (e.g., a linker, lipid, solubilizing group, and/or targeting ligand) described in WO 2012/030683.

In some embodiments, a provide oligonucleotide comprises an additional chemical moiety and/or a modification (e.g., of nucleobase, sugar, internucleotidic linkage, etc.) described in: U.S. Pat. Nos. 5,688,941; 6,294,664; 6,320,017; 6,576,752; 5,258,506; 5,591,584; 4,958,013; 5,082,830; 5,118,802; 5,138,045; 6,783,931; 5,254,469; 5,414,077; 5,486,603; 5,112,963; 5,599,928; 6,900,297; 5,214,136; 5,109,124; 5,512,439; 4,667,025; 5,525,465; 5,514,785; 5,565,552; 5,541,313; 5,545,730; 4,835,263; 4,876,335; 5,578,717; 5,580,731; 5,451,463; 5,510,475; 4,904,582; 5,082,830; 4,762,779; 4,789,737; 4,824,941; 4,828,979; 5,595,726; 5,214,136; 5,245,022; 5,317,098; 5,371,241; 5,391,723; 4,948,882; 5,218,105; 5,112,963; 5,567,810; 5,574,142; 5,578,718; 5,608,046; 4,587,044; 4,605,735; 5,585,481; 5,292,873; 5,552,538; 5,512,667; 5,597,696; 5,599,923; 7,037,646; 5,587,371; 5,416,203; 5,262,536; 5,272,250; or 8,106,022.

In some embodiments, an additional chemical moiety, e.g., a Mod, is connected via a linker. Various linkers are available in the art and may be utilized in accordance with the present disclosure, for example, those utilized for conjugation of various moieties with proteins (e.g., with antibodies to form antibody-drug conjugates), nucleic acids, etc. Certain useful linkers are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, and/or WO 2020/191252, the linker moieties of each which are independently incorporated herein by reference. In some embodiments, a linker is, as non-limiting examples, L001, L004, L009 or L010. In some embodiments, an oligonucleotide comprises a linker, but not an additional chemical moiety other than the linker. In some embodiments, an oligonucleotide comprises a linker, but not an additional chemical moiety other than the linker, wherein the linker is L001, L004, L009, or L010.

As demonstrated herein, provided technologies can provide high levels of activities and/or desired properties, in some embodiments, without utilizing particular structural elements (e.g., modifications, linkage configurations and/or patterns, etc.) reported to be desired and/or necessary (e.g., those reported in WO 2019/219581), though certain such structural elements may be incorporated into oligonucleotides in combination with various other structural elements in accordance with the present disclosure. For example, in some embodiments, oligonucleotides of the present disclosure have fewer nucleosides 3′ to a nucleoside opposite to a target nucleoside (e.g., a target adenosine), contain one or more phosphorothioate internucleotidic linkages at one or more positions where a phosphorothioate internucleotidic linkage was reportedly not favored or not allowed, contain one or more Sp phosphorothioate internucleotidic linkages at one or more positions where a Sp phosphorothioate internucleotidic linkage was reportedly not favored or not allowed, contain one or more Rp phosphorothioate internucleotidic linkages at one or more positions where a Rp phosphorothioate internucleotidic linkage was reportedly not favored or not allowed, and/or contain different modifications (e.g., internucleotidic linkage modifications, sugar modifications, etc.) and/or stereochemistry at one or more locations compared to those reportedly favorable or required for certain oligonucleotide properties and/or activities (e.g., presence of 2′-MOE, absence of phosphorothioate linkages at certain positions, absence of Sp phosphorothioate linkages at certain positions, and/or absence of Rp phosphorothioate linkages at certain positions were reportedly favorable or required for certain oligonucleotide properties and/or activities; as demonstrated herein, provided technologies can provide desired properties and/or high activities without utilizing 2′-MOE, without avoiding phosphorothioate linkages at one or more such certain positions, without avoiding Sp phosphorothioate linkages at one or more such certain positions, and/or without avoiding Rp phosphorothioate linkages at one or more such certain positions). Additionally or alternatively, provided oligonucleotides incorporates structural elements that were not previously recognized such as utilization of certain modifications (e.g., base modifications, sugar modifications (e.g., 2′-F), linkage modifications (e.g., non-negatively charged internucleotidic linkages), additional moieties, etc.) and levels, patterns, and combinations thereof.

For example, in some embodiments, as described herein, provided oligonucleotides contain no more than 5, 6, 7, 8, 9, 10, 11 or 12 nucleosides 3′ to a nucleoside opposite to a target nucleoside (e.g., a target adenosine).

Alternatively or additionally, as described herein (e.g., illustrated in certain Examples), for structural elements 3′ to a nucleoside opposite to a target nucleoside (e.g., a target adenosine), in some embodiments, about 50%-100% (e.g., about or at least about 50%, 55%, 60%, 65%, 70%, 75%, 80%, 85%, 90%, 95%) of internucleotidic linkages 3′ to a nucleoside opposite to a target nucleoside (e.g., a target adenosine) are each independently a modified internucleotidic linkage, which is optionally chirally controlled. In some embodiments, no more than 1, 2, or 3 internucleotidic linkages 3′ to a nucleoside opposite to a target nucleoside are natural phosphate linkages. In some embodiments, no such internucleotidic linkage is natural phosphate linkages. In some embodiments, no more than 1 such internucleotidic linkage is natural phosphate linkages. In some embodiments, no more than 2 such internucleotidic linkages are natural phosphate linkages. In some embodiments, no more than 3 such internucleotidic linkages are natural phosphate linkages. In some embodiments, each modified internucleotidic linkage is independently a phosphorothioate or a non-negatively charged internucleotidic linkage (e.g., n001). In some embodiments, each phosphorothioate internucleotidic linkage is chirally controlled. In some embodiments, no more than 1, 2, or 3 internucleotidic linkages 3′ to a nucleoside opposite to a target nucleoside are Rp phosphorothioate internucleotidic linkage. In some embodiments, an internucleotidic linkage bonded to a nucleoside opposite to a target nucleoside at the 3′-position of its sugar (considered a −1 position) is a Rp phosphorothioate internucleotidic linkage. In some embodiments, it is the only Rp phosphorothioate internucleotidic linkage 3′ to a nucleoside opposite to a target nucleoside. In some embodiments, an internucleotidic linkage at position −3 relative to a nucleoside opposite to a target nucleoside (e.g., for . . . N₀N₋₁N₋₂N₋₃ . . . , the internucleotidic linkage linking N₋₂ and N₋₃ wherein N₀ is a nucleoside opposite to a target nucleoside) is not a Rp phosphorothioate internucleotidic linkage. In some embodiments, an internucleotidic linkage at position −6 relative to a nucleoside opposite to a target nucleoside is not a Rp phosphorothioate internucleotidic linkage. In some embodiments, an internucleotidic linkage at position −4 and/or −5 relative to a nucleoside opposite to a target nucleoside is independently a modified internucleotidic linkage, e.g., a phosphorothioate internucleotidic linkage, or is independently a Rp phosphorothioate internucleotidic linkage. In some embodiments, one or more or all internucleotidic linkages at positions −1, −3, −4, −5, and −6 are each independently a Sp internucleotidic linkage. In some embodiments, one or more or all internucleotidic linkages at positions −1, −3, −4, −5, and −6 are each independently a Sp phosphorothioate internucleotidic linkage. In some embodiments, internucleotidic linkage(s) at position(s) −4 and/or −5 are each independently a Rp internucleotidic linkage. In some embodiments, internucleotidic linkage(s) at position(s) −4 and/or −5 are each independently a Rp phosphorothioate internucleotidic linkage. In many embodiments, no more than 1, 2, 3, 4, or 5 internucleotidic linkages are Rp phosphorothioate internucleotidic linkage.

Alternatively or additionally, as described herein (e.g., illustrated in certain Examples), in some embodiments, about 50%-100% (e.g., about or at least about 50%, 55%, 60%, 65%, 70%, 75%, 80%, 85%, 90%, or 95%) of internucleotidic linkages 5′ to a nucleoside opposite to a target nucleoside (e.g., a target adenosine) are each independently a modified internucleotidic linkage, which is optionally chirally controlled. In some embodiments, no or no more than 1, 2, or 3 internucleotidic linkages 5′ to a nucleoside opposite to a target nucleoside (e.g., a target adenosine) are not modified internucleotidic linkages. In some embodiments, no or no more than 1, 2, or 3 internucleotidic linkages 5′ to a nucleoside opposite to a target nucleoside (e.g., a target adenosine) are not phosphorothioate internucleotidic linkages. In some embodiments, no or no more than 1, 2, or 3 internucleotidic linkages 5′ to a nucleoside opposite to a target nucleoside (e.g., a target adenosine) are not Sp phosphorothioate internucleotidic linkages. In some embodiments, no more than 1, 2, or 3 internucleotidic linkages 5′ to a nucleoside opposite to a target nucleoside (e.g., a target adenosine) are natural phosphate linkages. In some embodiments, no such internucleotidic linkage is natural phosphate linkages. In some embodiments, no more than 1 such internucleotidic linkage is natural phosphate linkages. In some embodiments, no more than 2 such internucleotidic linkages are natural phosphate linkages. In some embodiments, no more than 3 such internucleotidic linkages are natural phosphate linkages. In some embodiments, each modified internucleotidic linkage is independently a phosphorothioate or a non-negatively charged internucleotidic linkage (e.g., n001). In some embodiments, there are no 2, 3, or 4 consecutive internucleotidic linkages 5′ to a nucleoside opposite to a target nucleoside, each of which is not a phosphorothioate internucleotidic linkage. In some embodiments, there are no 2, 3, or 4 consecutive internucleotidic linkages 5′ to a nucleoside opposite to a target nucleoside, each of which is chirally controlled and is not a Sp phosphorothioate internucleotidic linkage. In some embodiments, no or no more than 1, 2, 3, 4, or 5 internucleotidic linkages 5′ to a nucleoside opposite to a target nucleoside (e.g., a target adenosine) are Rp phosphorothioate internucleotidic linkage. In some embodiments, an internucleotidic linkage bonded to a nucleoside opposite to a target nucleoside at the 5′-position of its sugar (considered a +1 position) is a Rp phosphorothioate internucleotidic linkage. In some embodiments, it is the only Rp phosphorothioate internucleotidic linkage 3′ to a nucleoside opposite to a target nucleoside. In some embodiments, an internucleotidic linkage at position +5 relative to a nucleoside opposite to a target nucleoside (e.g., for . . . N₊₅N₊₄N₊₃N₊₂N₊₁N₀ . . . , the internucleotidic linkage linking N₊₄ and N₊₅ wherein N₀ is a nucleoside opposite to a target nucleoside) is not a Rp phosphorothioate internucleotidic linkage. In some embodiments, an internucleotidic linkage at positions +11 is not a Sp phosphorothioate internucleotidic linkage. In some embodiments, one or more or all internucleotidic linkages at positions +6 to +8 relative to a nucleoside opposite to a target nucleoside are each independently a modified internucleotidic linkage, optionally chirally controlled. In some embodiments, each of them is independently a phosphorothioate internucleotidic linkage. In some embodiments, each of them is independently a Sp phosphorothioate internucleotidic linkage. In some embodiments, one or more or all internucleotidic linkages at positions +6 to +8 relative to a nucleoside opposite to a target nucleoside are each independently a phosphorothioate internucleotidic linkage, optionally chirally controlled. In some embodiments, one or more or all internucleotidic linkages at positions +6, +7, +8, +9, and +11 are each independently Rp internucleotidic linkages. In some embodiments, one or more or all internucleotidic linkages at positions +6, +7, +8, +9, and +11 are each independently Rp phosphorothioate internucleotidic linkages. In some embodiments, one or more or all internucleotidic linkages at positions +5, +6, +7, +8, and +9 relative to a nucleoside opposite to a target adenosine are each independently Sp internucleotidic linkages. In some embodiments, one or more or all internucleotidic linkages at positions +5, +6, +7, +8, and +9 relative to a nucleoside opposite to a target adenosine are each independently Sp phosphorothioate internucleotidic linkages. In some embodiments, an internucleotidic linkage at position +5 is a Sp internucleotidic linkage. In some embodiments, an internucleotidic linkage at position +5 is a Sp phosphorothioate internucleotidic linkage. In some embodiments, an internucleotidic linkage at position +6 is a Sp internucleotidic linkage. In some embodiments, an internucleotidic linkage at position +6 is a Sp phosphorothioate internucleotidic linkage. In some embodiments, an internucleotidic linkage at position +7 is a Sp internucleotidic linkage. In some embodiments, an internucleotidic linkage at position +7 is a Sp phosphorothioate internucleotidic linkage. In some embodiments, an internucleotidic linkage at position +8 is a Sp internucleotidic linkage. In some embodiments, an internucleotidic linkage at position +8 is a Sp phosphorothioate internucleotidic linkage. In some embodiments, an internucleotidic linkage at position +9 is a Sp internucleotidic linkage. In some embodiments, an internucleotidic linkage at position +9 is a Sp phosphorothioate internucleotidic linkage. In some embodiments, at least about 1, 2, 3, 4, 5, 6, 7, 8, 9, or 10, or about 1, 2, 3, 4, 5, 6, 7, 8, 9, 10, 11, 12, 13, 14, 15, 16, 18, 19, 20, 21, 22, 23, 24, 25, 26, 27, 28, 29, 30, 31, or 32, or about 50%-100% (e.g., about or at least about 50%, 55%, 60%, 65%, 70%, 75%, 80%, 85%, 90%, or 95%) of internucleotidic linkages 5′ to a nucleoside opposite to a target nucleoside (e.g., a target adenosine) are each independently chirally controlled and a Sp internucleotidic linkage. In some embodiments, at least about 1, 2, 3, 4, 5, 6, 7, 8, 9, or 10, or about 1, 2, 3, 4, 5, 6, 7, 8, 9, 10, 11, 12, 13, 14, 15, 16, 18, 19, 20, 21, 22, 23, 24, 25, 26, 27, 28, 29, 30, 31, or 32, or about 50%-100% (e.g., about or at least about 50%, 55%, 60%, 65%, 70%, 75%, 80%, 85%, 90%, or 95%) of phosphorothioate internucleotidic linkages 5′ to a nucleoside opposite to a target nucleoside (e.g., a target adenosine) are each independently chirally controlled and are Sp. In some embodiments, each phosphorothioate internucleotidic linkages 5′ to a nucleoside opposite to a target nucleoside (e.g., a target adenosine) is chirally controlled. In some embodiments, each phosphorothioate internucleotidic linkages 5′ to a nucleoside opposite to a target nucleoside (e.g., a target adenosine) is Sp.

### Production of Oligonucleotides and Compositions

Various methods can be utilized for production of oligonucleotides and compositions and can be utilized in accordance with the present disclosure. For example, traditional phosphoramidite chemistry (e.g., phosphoramidites comprising —CH₂CH₂CN and —N(i-Pr)₂) can be utilized to prepare stereorandom oligonucleotides and compositions, and certain reagents and chirally controlled technologies can be utilized to prepare chirally controlled oligonucleotide compositions, e.g., as described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, and/or WO 2020/191252, the reagents and methods of each of which is incorporated herein by reference.

In some embodiments, chirally controlled/stereoselective preparation of oligonucleotides and compositions thereof comprise utilization of a chiral auxiliary, e.g., as part of monomeric phosphoramidites. Examples of such chiral auxiliary reagents and phosphoramidites are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, and/or WO 2020/191252, the chiral auxiliary reagents and phosphoramidites of each of which are independently incorporated herein by reference. In some embodiments, a chiral auxiliary is a chiral auxiliary described in any of: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the chiral auxiliaries of each of which are independently incorporated herein by reference.

In some embodiments, chirally controlled preparation technologies, including oligonucleotide synthesis cycles, reagents and conditions are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, and/WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, WO 2019/032612, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the oligonucleotide synthesis methods, cycles, reagents and conditions of each of which are independently incorporated herein by reference.

Once synthesized, provided oligonucleotides and compositions are typically further purified. Suitable purification technologies are widely known and practiced by those skilled in the art, including but not limited to those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, and/or WO 2020/191252, the purification technologies of each of which are independently incorporated herein by reference.

In some embodiments, a cycle comprises or consists of coupling, capping, modification and deblocking. In some embodiments, a cycle comprises or consists of coupling, capping, modification, capping and deblocking. These steps are typically performed in the order they are listed, but in some embodiments, as appreciated by those skilled in the art, the order of certain steps, e.g., capping and modification, may be altered. If desired, one or more steps may be repeated to improve conversion, yield and/or purity as those skilled in the art often perform in syntheses. For example, in some embodiments, coupling may be repeated; in some embodiments, modification (e.g., oxidation to install ═O, sulfurization to install ═S, etc.) may be repeated; in some embodiments, coupling is repeated after modification which can convert a P(III) linkage to a P(V) linkage which can be more stable under certain circumstances, and coupling is routinely followed by modification to convert newly formed P(III) linkages to P(V) linkages. In some embodiments, when steps are repeated, different conditions may be employed (e.g., concentration, temperature, reagent, time, etc.).

Technologies for formulating provided oligonucleotides and/or preparing pharmaceutical compositions, e.g., for administration to subjects via various routes, are readily available in the art and can be utilized in accordance with the present disclosure, e.g., those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194 and references cited therein.

Technologies for formulating provided oligonucleotides and/or preparing pharmaceutical compositions, e.g., for administration to subjects via various routes, are readily available in the art and can be utilized in accordance with the present disclosure, e.g., those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194 and references cited therein.

In some embodiments, a useful chiral auxiliary has the structure of

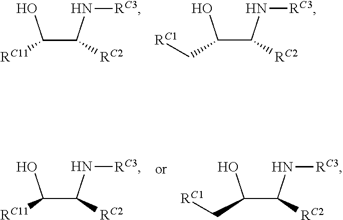

or a salt thereof, wherein R^{C11} is -L^{C1}-R^{C1}, L^{C1} is optionally substituted —CH₂—. R^{C1} is R, —Si(R)₃, —SO₂R or an electron-withdrawing group, and R^{C2} and R^{C3} are taken together with their intervening atoms to form an optionally substituted 3-10 membered saturated ring having, in addition to the nitrogen atom, 0-2 heteroatoms. In some embodiments, a useful chiral auxiliary has the structure of

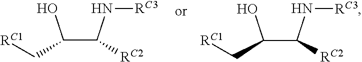

wherein R^{C1} is R, —Si(R)₃ or −SO₂R, and R^{C2} and R^{C3} are taken together with their intervening atoms to form an optionally substituted 3-7 membered saturated ring having, in addition to the nitrogen atom, 0-2 heteroatoms. is a formed ring is an optionally substituted 5-membered ring. In some embodiments, a useful chiral auxiliary has the structure of

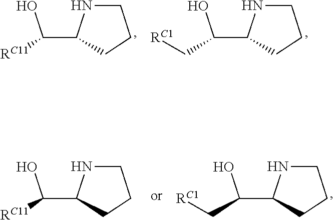

or a salt thereof. In some embodiments, a useful chiral auxiliary has the structure of

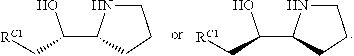

In some embodiments, a useful chiral auxiliary is a DPSE chiral auxiliary. In some embodiments, purity or stereochemical purity of a chiral auxiliary is at least 85%, 90%, 95%, 96%, 97%, 98%, or 99%. In some embodiments, it is at least 85%. In some embodiments, it is at least 90%. In some embodiments, it is at least 95%. In some embodiments, it is at least 96%. In some embodiments, it is at least 97%. In some embodiments, it is at least 98%. In some embodiments, it is at least 99%.

In some embodiments, L^{C1} is —CH₂—. In some embodiments, L^{C1} is substituted —CH₂—. In some embodiments, L^{C1} is monosubstituted —CH₂—.

In some embodiments, R^{C1} is R. In some embodiments, R^{C1} is optionally substituted phenyl. In some embodiments, R^{C1} is —SiR₃. In some embodiments, R^{C1} is —SiPh₂Me. In some embodiments, R^{C1} is —SO₂R. In some embodiments, R is not hydrogen. In some embodiments, R is optionally substituted phenyl. In some embodiments, R is phenyl. In some embodiments, R is optionally substituted C₁₋₆ aliphatic. In some embodiments, R is C₁₋₆ alkyl. In some embodiments, R is methyl. In some embodiments, R is t-butyl.

In some embodiments, R^{C1} is an electron-withdrawing group, such as —C(O)R, —OP(O)(OR)₂, —OP(O)(R)₂, —P(O)(R)₂, —S(O)R, —S(O)₂R, etc. In some embodiments, chiral auxiliaries comprising electron-withdrawing group R^{C1} groups are particularly useful for preparing chirally controlled non-negatively charged internucleotidic linkages and/or chirally controlled internucleotidic linkages bonded to natural RNA sugar.

In some embodiments, R^{C2} and R^{C3} are taken together with their intervening atoms to form an optionally substituted 3-10 (e.g., 3, 4, 5, 6, 7, 8, 9, or 10) membered saturated ring having no heteroatoms in addition to the nitrogen atom. In some embodiments, R^{C2} and R^{C3} are taken together with their intervening atoms to form an optionally substituted 5-membered saturated ring having no heteroatoms in addition to the nitrogen atom.

In some embodiments, methods for preparing oligonucleotides and/or compositions comprise using a chiral auxiliary described herein, e.g., for constructing one or more chirally controlled internucleotidic linkages. In some embodiments, one or more chirally controlled internucleotidic linkages are independently constructed using a DPSE chiral auxiliary. In some embodiments, each chirally controlled phosphorothioate internucleotidic linkage is independently constructed using a DPSE chiral auxiliary. In some embodiments, one or more chirally controlled internucleotidic linkages are independently constructed using

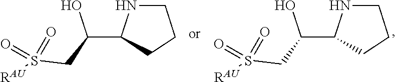

or a salt thereof, wherein R^{AU} is as described herein. In some embodiments, each chirally controlled non-negatively charged internucleotidic linkage (e.g., n001) is independently constructed using

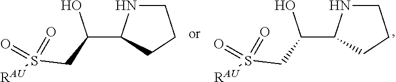

or a salt thereof. In some embodiments, each chirally controlled internucleotidic linkage is independently constructed using

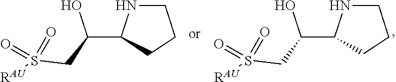

or a salt thereof. In some embodiments, R^{AU} is optionally substituted C₁₋₂₀, C₁₋₁₀, C₁₋₆, C₁₋₅, or C₁₋₄ aliphatic. In some embodiments, R^{AU} is optionally substituted C₁₋₂₀, C₁₋₁₀, C₁₋₆, C₁₋₅, or C₁₋₄ alkyl. In some embodiments, RAU is optionally substituted aryl. In some embodiments, R^{AU} is phenyl. In some embodiments, one or more chirally controlled internucleotidic linkages are constructed using a PSM chiral auxiliary. In some embodiments, each chirally controlled non-negatively charged internucleotidic linkage (e.g., n001) is independently constructed using a PSM chiral auxiliary. In some embodiments, each chirally controlled internucleotidic linkages is independently constructed using a PSM chiral auxiliary. As appreciated by those skilled in the art, a chiral auxiliary is often utilized in a phosphoramidite (e.g.,

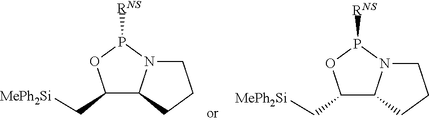

(DPSE phosphoramidites),

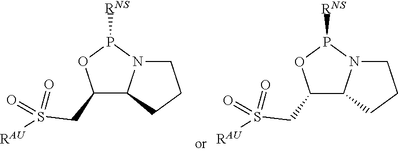

(wherein R^{AU} is independently as described herein; when R^{AU} is -Ph, PSM phosphoramidites), wherein R^{NS} is an optionally substituted/protected nucleoside (e.g., optionally protected for oligonucleotide synthesis), or a salt thereof, etc.) for oligonucleotide preparation. In some embodiments, a method comprises providing a DPSE and/or a PSM phosphoramidite or a salt thereof. In some embodiments, a provided method comprises contacting a DPSE and/or a PSM phosphoramidite or a salt thereof with —OH (e.g., 5′-OH of a nucleoside or an oligonucleotide chain). As those skilled in the art appreciate, contacting can be performed under various suitable conditions so that a phosphorus linkage is formed. In some embodiments, preparation of each chirally controlled internucleotidic linkage independently comprises contacting a DPSE or PSM phosphoramidite or a salt thereof with —OH (e.g., 5′-OH of a nucleoside or an oligonucleotide chain). In some embodiments, preparation of each chirally controlled phosphorothioate internucleotidic linkage independently comprises contacting a DPSE phosphoramidite or a salt thereof with —OH (e.g., 5′-OH of a nucleoside or an oligonucleotide chain). In some embodiments, preparation of each chirally controlled non-negatively charged internucleotidic linkage (e.g., n001) independently comprises contacting a PSM phosphoramidite or a salt thereof with —OH (e.g., 5′-OH of a nucleoside or an oligonucleotide chain). In some embodiments, preparation of each chirally controlled internucleotidic linkage independently comprises contacting a PSM phosphoramidite or a salt thereof with —OH (e.g., 5′-OH of a nucleoside or an oligonucleotide chain). In some embodiments, contacting forms a P(III) linkage comprising a phosphorus atom bonded to two sugars and a chiral auxiliary moiety (e.g.,

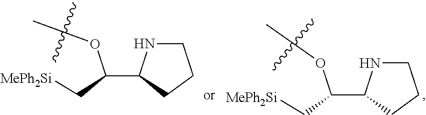

or a salt form thereof (e.g., from DPSE phosphoramidites or salts thereof),

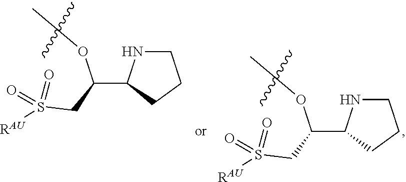

or a salt form thereof (wherein R^{AU} is independently as described herein; when R^{AU} is -Ph, e.g., from PSM phosphoramidites or salts thereof), etc.). In some embodiments, an oligonucleotide comprises a P(III) linkage comprising a chiral auxiliary moiety, e.g., from a DPSE or PSM phosphoramidite. In some embodiments, a P(III) linkage comprising a chiral auxiliary moiety is chirally controlled. In some embodiments, a chiral auxiliary moiety may be protected, e.g., before converting a P(III) linkage to a P(V) linkage (e.g., before sulfurization, reacting with azide, etc.). In some embodiments, a protected chiral auxiliary has the structure of

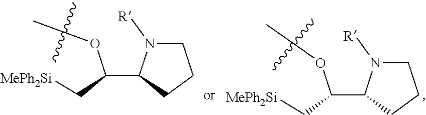

or a salt form thereof (e.g., wherein R′ is independently as described herein; e.g., from DPSE phosphoramidites or salts thereof), or

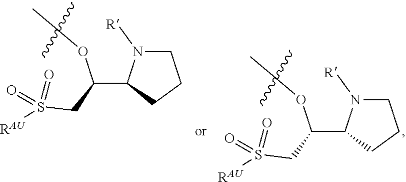

or a salt form thereof (wherein each R′ and R^{AU} is independently as described herein; when R^{AU} is -Ph, e.g., from PSM phosphoramidites or salts thereof), wherein each R′ is independently as described herein. In some embodiments, R′ is —C(O)R, wherein R is as described herein. In some embodiments, R is —CH₃. In some embodiments, an oligonucleotide comprises a protected chiral auxiliary. In some embodiments, each chirally controlled internucleotidic linkage in an oligonucleotide independently comprises

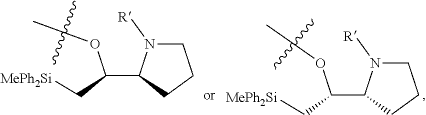

or a salt form thereof, or

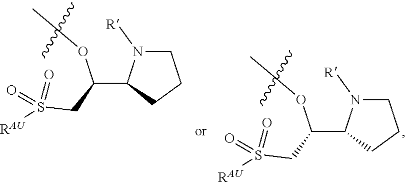

or a salt form thereof. In some embodiments, each chirally controlled internucleotidic linkage in an oligonucleotide independently comprises

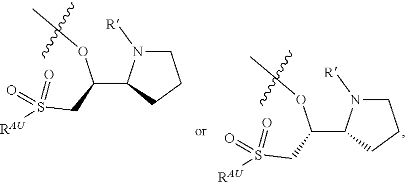

or a salt form thereof. In some embodiments, R′ is —C(O)R. In some embodiments, R′ is —C(O)CH₃. In some embodiments, R^{AU} is Ph. In some embodiments, an oligonucleotide comprises one or more

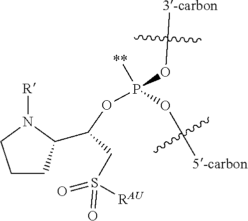

or a salt form thereof (PIII-1), wherein each variable independently as described herein. In some embodiments, an oligonucleotide comprises one or more

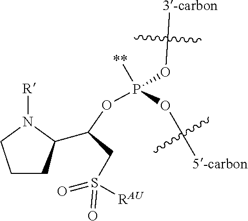

or a salt form thereof (PIII-2), wherein each variable independently as described herein. In some embodiments, an oligonucleotide comprises one or more

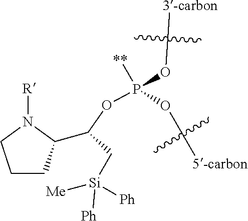

or a salt form thereof (PIII-5), wherein each variable independently as described herein. In some embodiments, an oligonucleotide comprises one or more

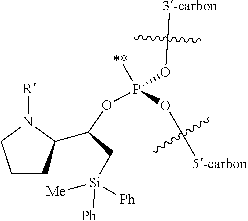

or a salt form thereof (PIII-6), wherein each variable independently as described herein. In some embodiments, a 5′-end internucleotidic linkage is PIII-1, PIII-2, PI11-5, or PIII-6. In some embodiments, a 5′-end internucleotidic linkage is PIII-1 or PIII-2. In some embodiments, R′ is —H. In some embodiments, R′ is —C(O)R. In some embodiments, R′ is —C(O)CH₃. In some embodiments, R^{AU} is -Ph. In some embodiments, a P(III) linkage is converted into a P(V) linkage. In some embodiments, a P(V) linkage comprises a phosphorus atom bonded to two sugars, a chiral auxiliary moiety (e.g.,

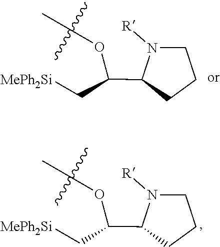

or a salt form thereof (wherein R′ is as described herein; e.g., from DPSE phosphoramidites or salts thereof),

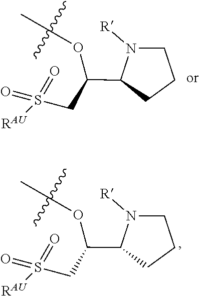

or a salt form thereof (wherein each of R′ and R^{AU} is independently as described herein; when R^{AU} is -Ph, e.g., from PSM phosphoramidites or salts thereof), etc.), and S or

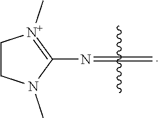 [SMILES: *=NC1=[N+](C)CCN1C]

In some embodiments, a P(V) linkage comprises a phosphorus atom bonded to two sugars,

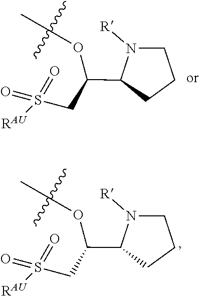

or a salt form thereof (wherein each R′ and R^{AU} is independently as described herein; when R^{AU} is -Ph, e.g., from PSM phosphoramidites or salts thereof), etc.), and S or

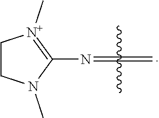 [SMILES: *=NC1=[N+](C)CCN1C]

In some embodiments, a P(V) linkage comprises a phosphorus atom bonded to two sugars,

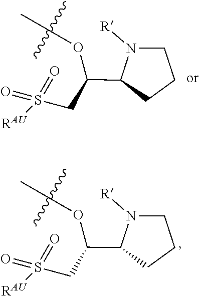

or a salt form thereof (wherein each R′ and R^{AU} is independently as described herein; when R^{AU} is -Ph, e.g., from PSM phosphoramidites or salts thereof), etc.), and S. In some embodiments, a P(V) linkage comprises a phosphorus atom bonded to two sugars,

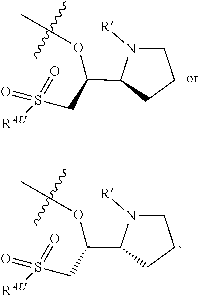

or a salt form thereof (wherein each R′ and R^{AU} is independently as described herein; when R^{AU} is -Ph, e.g., from PSM phosphoramidites or salts thereof), etc.), and

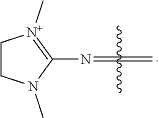 [SMILES: *=NC1=[N+](C)CCN1C]

Those skilled in the art will appreciate that

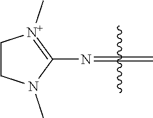 [SMILES: *=NC1=[N+](C)CCN1C]

can exist with a counterion, e.g., in some embodiments, PF₆⁻. In some embodiments, an oligonucleotide comprises one or more

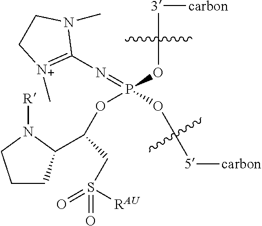

or a salt form thereof (PV-1), wherein each variable independently as described herein. In some embodiments, an oligonucleotide comprises one or more

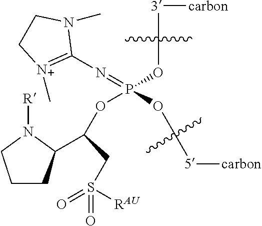

or a salt form thereof (PV-2), wherein each variable independently as described herein. In some embodiments, an oligonucleotide comprises one or more

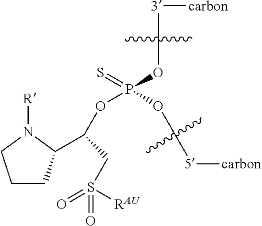

or a salt form thereof (PV-3), wherein each variable independently as described herein. In some embodiments, an oligonucleotide comprises one or more

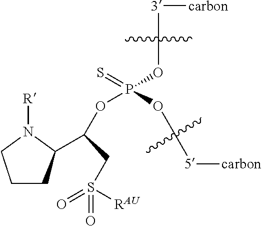

or a salt form thereof (PV-4), wherein each variable independently as described herein. In some embodiments, an oligonucleotide comprises one or more

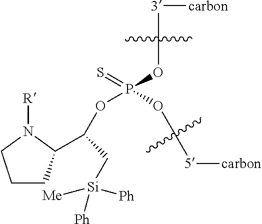

or a salt form thereof (PV-5), wherein each variable independently as described herein. In some embodiments, an oligonucleotide comprises one or more

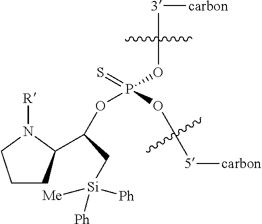

or a salt form thereof (PV-6), wherein each variable independently as described herein. In some embodiments, each chiral internucleotidic linkage, or each chirally controlled internucleotidic linkage, of an oligonucleotide is independently selected from PIII-1, PIII-2, PIII-5, PIII-6, PV-1, PV-2, PV-3, PV-4, PV-5, and PV-6. In some embodiments, each chiral internucleotidic linkage, or each chirally controlled internucleotidic linkage, of an oligonucleotide is independently selected from PIII-1, PIII-2, PV-1, PV-2, PV-3, and PV-4. In some embodiments, a linkage of PIII-1, PIII-2, PIII-5, or PIII-6 is typically the 5′-end internucleotidic linkage. In some embodiments, each chiral internucleotidic linkage, or each chirally controlled internucleotidic linkage, of an oligonucleotide is independently selected from PV-1, PV-2, PV-3, PV-4, PV-5, and PV-6. In some embodiments, each chiral internucleotidic linkage, or each chirally controlled internucleotidic linkage, of an oligonucleotide is independently selected from PV-1, PV-2, PV-3, or PV-4. In some embodiments, a provided oligonucleotide is an oligonucleotide as described herein, e.g., of Table A1, wherein each *S is independently replaced with PV-3 or PV-5, each *R is independently replaced with PV-4 or PV-6, each n001R is independently replaced with PV-1, and each n001S is independently replaced with PV-2. In some embodiments, a provided oligonucleotide is an oligonucleotide as described herein, e.g., of Table A1, wherein each *S is independently replaced with PV-3, each *R is independently replaced with PV-4, each n001R is independently replaced with PV-1, and each n001S is independently replaced with PV-2. In some embodiments, each natural phosphate linkage is independently replaced with a precursor, e.g.,

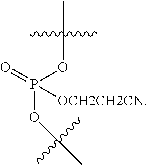

In some embodiments, R′ is —H. In some embodiments, R′ is —C(O)R. In some embodiments, R′ is —C(O)CH₃. In some embodiments, R^{AU} is -Ph. In some embodiments, a method comprises removal of one or more chiral auxiliary moieties so that phosphorothioate and/or non-negatively charged internucleotidic linkages (e.g., n001) are formed (e.g., from V-1, PV-2, PV-3, PV-4, PV-5, PV-6, etc.). In some embodiments, removal of a chiral auxiliary (e.g., PSM) comprises contacting an oligonucleotide with a base (e.g., N(R)₃ such as DEA) under anhydrous conditions.

In some embodiments, as appreciated by those skilled in the art, for preparation of a chirally controlled internucleotidic linkage, a phosphoramidite (e.g., a DPSE or PSM phosphoramidite), is typically utilized in a chirally enriched or pure form (e.g., of a purity as described herein (e.g., about or at least about 90%, 91%, 92%, 93%, 94%, 95%, 96%, 97%, 98%, or 99%, or about 100%)).

In some embodiments, the present disclosure provides useful reagents for preparation of oligonucleotides and compositions thereof. In some embodiments, phosphoramidites comprise nucleosides, nucleobases and sugars as described herein. In some embodiments, nucleobases and sugars are properly protected for oligonucleotide synthesis as those skilled in the art will appreciate. In some embodiments, a phosphoramidite has the structure of R^{NS}—P(OR)N(R)₂, wherein R^{NS} is a optionally protected nucleoside moiety. In some embodiments, a phosphoramidite has the structure of R^{NS}—P(OCH₂CH₂CN)N(i-Pr)₂. In some embodiments, a phosphoramidite comprises a nucleobase which is or comprises Ring BA, wherein Ring BA has the structure of BA-I, BA-I-a, BA-I-b, BA-II, BA-II-a, BA-II-b, BA-III, BA-III-a, BA-III-b, BA-IV, BA-IV-a, BA-IV-b, BA-V, BA-V-a, BA-V-b, or BA-VI, or a tautomer of Ring BA, wherein the nucleobase is optionally substituted or protected. In some embodiments, a phosphoramidite comprises a chiral auxiliary moiety, wherein the phosphorus is bonded to an oxygen and a nitrogen atom of the chiral auxiliary moiety. In some embodiments, a phosphoramidite has the structure of

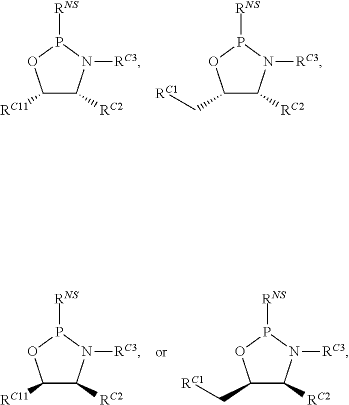

or a salt thereof, wherein R^{NS} is a protected nucleoside moiety (e.g., 5′-OH and/or nucleobases suitably protected for oligonucleotide synthesis), and each other variable is independently as described herein. In some embodiments, a phosphoramidite has the structure of

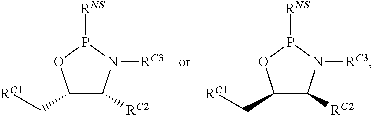

wherein R^{NS} is a protected nucleoside moiety (e.g., 5′-OH and/or nucleobases suitably protected for oligonucleotide synthesis), R^{C1} is R, —Si(R)₃ or —SO₂R, and R^{C2} and R^{C3} are taken together with their intervening atoms to form an optionally substituted 3-7 membered saturated ring having, in addition to the nitrogen atom, 0-2 heteroatoms, wherein the coupling forms an internucleotidic linkage. In some embodiments, 5′-OH of R^{NS} is protected. In some embodiments, 5′-OH of R^{NS} is protected as —ODMTr. In some embodiments, R^{NS} is bonded to phosphorus through its 3′-O—. In some embodiments, a formed ring by R^{C2} and R^{C3} is an optionally substituted 5-membered ring. In some embodiments, a phosphoramidite has the structure of

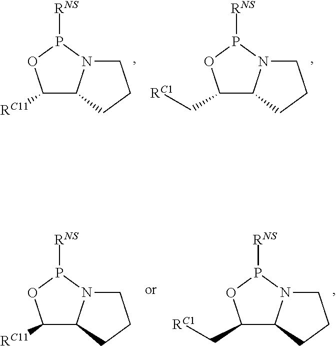

or a salt thereof. In some embodiments, a phosphoramidite has the structure of

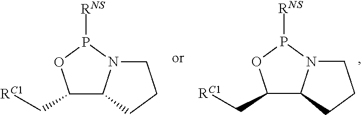

In some embodiments, as described herein R^{NS} comprises a modified nucleobase (e.g., b001A, b002A, b003A, b008U, b001C, etc.) which is optionally protected for oligonucleotide synthesis.

In some embodiments, purity or stereochemical purity of a phosphoramidite is at least 85%, 90%, 95%, 96%, 97%, 98%, or 99%. In some embodiments, it is at least 85%. In some embodiments, it is at least 90%. In some embodiments, it is at least 95%.

In some embodiments, the present disclosure provides a method for preparing an oligonucleotide or composition, comprising coupling a free —OH, e.g., a free 5′-OH, of an oligonucleotide or a nucleoside with a phosphoramidite as described herein.

In some embodiments, the present disclosure provides an oligonucleotide, wherein the oligonucleotide comprises one or more modified internucleotidic linkages each independently having the structure of —O⁵—P^{L}(W)(R^{CA})—O³—, wherein:

P^{L} is P, or P(═W);

W is O, S, or W^{N};

W^{N} is ═N—C(—N(R¹)₂═N⁺(R¹)₂Q⁻;

Q⁻ is an anion;

R^{CA} is or comprises an optionally capped chiral auxiliary moiety,

O⁵ is an oxygen bonded to a 5′-carbon of a sugar, and

O³ is an oxygen bonded to a 3′-carbon of a sugar.

In some embodiments, a modified internucleotidic linkage is optionally chirally controlled. In some embodiments, a modified internucleotidic linkage is optionally chirally controlled.

In some embodiments, a provided methods comprising removing R^{CA} from such a modified internucleotidic linkages. In some embodiments, after removal, bonding to R^{CA} is replaced with —OH. In some embodiments, after removal, bonding to R^{CA} is replaced with ═O, and bonding to W^{N} is replaced with —N═C(N(R¹)₂)₂.

In some embodiments, P^{L} is P═S, and when R^{CA} is removed, such an internucleotidic linkage is converted into a phosphorothioate internucleotidic linkage.

In some embodiments, P^{L} is P═W^{N}, and when R^{CA} is removed, such an internucleotidic linkage is converted into an internucleotidic linkage having the structure of

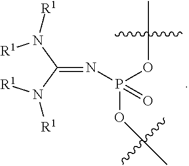

In some embodiments, an internucleotidic linkage having the structure of

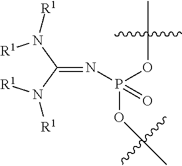

has the structure of

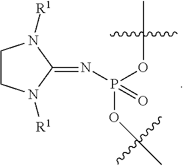

In some embodiments, an internucleotidic linkage having the structure of

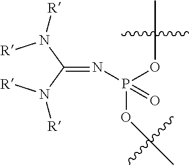

has the structure of

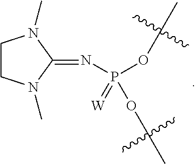

In some embodiments, P^{L} is P (e.g., in newly formed internucleotidic linkage from coupling of a phosphoramidite with a 5′-OH). In some embodiments, W is O or S. In some embodiments, W is S (e.g., after sulfurization). In some embodiments, W is O (e.g., after oxidation). In some embodiments, certain non-negatively charged internucleotidic linkages or neutral internucleotidic linkages may be prepared by reacting a P(III) phosphite triester internucleotidic linkage with azido imidazolinium salts (e.g., compounds comprising

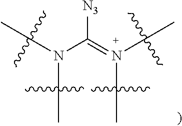

under suitable conditions. In some embodiments, an azido imidazolinium salt is a salt of PF₆⁻. In some embodiments, an azido imidazolinium salt is a slat of

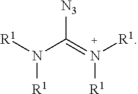

In some embodiments, an azido imidazolinium salt is 2-azido-1,3-dimethylimidazolinium hexafluorophosphate.

As appreciated by those skilled in the art, Q⁻ can be various suitable anion present in a system (e.g., in oligonucleotide synthesis), and may vary during oligonucleotide preparation processes depending on cycles, process stages, reagents, solvents, etc. In some embodiments, Q⁻ is PF₆⁻.

In some embodiments, R^{CA} is

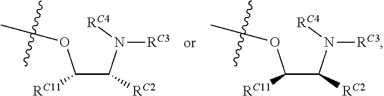

wherein R^{C4} is —H or —C(O)R′, and each other variable is independently as described herein. In some embodiments, R^{CA} is

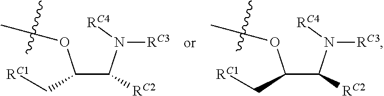

wherein R^{C1} is R, —Si(R)₃ or —SO₂R, R^{C2} and R^{C3} are taken together with their intervening atoms to form an optionally substituted 3-7 membered saturated ring having, in addition to the nitrogen atom, 0-2 heteroatoms, R^{C4} is —H or —C(O)R′. In some embodiments, R^{C4} is —H. In some embodiments, R^{C4} is —C(O)CH₃. In some embodiments, R^{C2} and R^{C3} are taken together to form an optionally substituted 5-membered ring.

In some embodiments, R^{C4} is —H (e.g., in n newly formed internucleotidic linkage from coupling of a phosphoramidite with a 5′-OH). In some embodiments, R^{C4} is —C(O)R (e.g., after capping of the amine). In some embodiments, R is methyl.

In some embodiments, each chirally controlled phosphorothioate internucleotidic linkage is independently converted from —O⁵—P^{L}(W)(R^{CA})—O³—.

### ADAR

Among other things, provided technologies can provide modification/editing of target adenosine by converting A to I. In some embodiments, oligonucleotides and/or duplexes formed by oligonucleotides with target nucleic acids interact with proteins, e.g., ADAR proteins. In some embodiments, such proteins comprise adenosine modifying activities and can modify target adenosine in target nucleic acids, e.g., converting them to inosine.

ADAR proteins are naturally expressed proteins in various cells, tissues, organs and/or organism. It has been reported that some ADAR proteins, e.g., ADAR1 and ADAR2, can edit adenosine through deamination, converting adenosine to inosine which can provide a number of functions including being read as or similar to G during translation. Mechanism of ADAR-mediated mRNA editing (e.g., deamination) has been reported. For example, ADAR proteins are reported to catalyze conversion of adenosine to inosine on double-stranded RNA substrates with mismatches. As appreciated by those skilled in the art, inosine can be recognized as guanosine by cellular translation and/or splicing machinery. ADAR can thus be used for functional adenosine to guanosine editing of nucleic acids, e.g., pre-mRNA and mRNA substrates.

In some embodiments, the present disclosure provides oligonucleotides and compositions thereof for ADAR-mediated editing of target adenosine in target nucleic acids, e.g. RNA. ADAR-mediated RNA-editing can offer several advantages over DNA-editing, e.g., delivery is simplified as expression of recombinant proteins like Cas9 is not required. Both ADAR1 and ADAR2 are endogenous enzymes, so cellular delivery of oligonucleotides alone can be sufficient for editing. Off-target effects, if any, are transient and changes are not made to genomic DNA. Additionally, ADAR-mediated editing can be used in post-mitotic cells and it does not require an HDR-template for repair. Three vertebrate ADAR genes have been reported with common functional domains (Nishikura Nat Rev Mol Cell Biol. 2016 February; 17(2): 83-96; Nishikura Annu Rev Biochem. 2010; 79: 321-349; Thomas and Beal Bioessays. 2017 April; 39(4)). All 3 ADARs contain a dsRNA-binding domains (dsRBD), which can contact dsRNA substrates. Some ADAR1 also contains Z-DNA-binding domains. ADAR1 has been reported to expressed significantly in brain, lung, kidney, liver, and heart, etc., and may occur in two isoforms. In some embodiments, isoform p150 can be induced by interferon while isoform p110 can be constitutively expressed. In some embodiments, it can be beneficial to utilize p110 as it is reported to be ubiquitously and constitutively expressed. ADAR2 can be highly expressed, e.g. in the brain and lungs, and is reported to be exclusively localized to the nucleus. ADAR3 is reported to be catalytically inactive and expressed only in the brain. Potential differences in tissue expression can be taken into consideration when choosing a therapeutic target.

Use of oligonucleotides for RNA editing by ADAR has been reported. Among other things, the present disclosure recognizes that previously reported technologies generally suffer one or more disadvantages, such as low stability (e.g., oligonucleotides with natural RNA sugars), low editing efficiency, low editing specificity (e.g., a number of As are edited in a portion of a target nucleic acid substantially complementary to an oligonucleotide), specific structures in oligonucleotides for ADAR recognition/recruitment, exogenous proteins (e.g., those engineered to recognize oligonucleotides with specific structures and/or duplexes thereof (e.g., with target nucleic acids) for editing), etc. Additionally, previously reported technologies typically utilize stereorandom oligonucleotide compositions when oligonucleotides comprise one or more chiral linkage phosphorus of modified internucleotidic linkages.

For example, various reported oligonucleotides contain ADAR-recruiting domains. Merkle et al., Nat Biotechnol. 2019 February; 37(2):133-138 disclosed oligonucleotides comprising an imperfect 20-bp hairpin ADAR-recruiting domain that is an intramolecular stem loop to recruit endogenous human ADAR2 to edit endogenous transcript. Oligonucleotides reported in Mali et al., Nat Methods. 2019 March; 16(3):239-242 contain ADAR substrate GluR2 pre-messenger RNA sequences or MS2 hairpins in addition to specificity domains that hybridize to the target mRNA.

Certain reported editing approach utilizes exogenous or engineered proteins, e.g., those utilizing CRISPR/Cas9 system. For example, Komor et al. Nature 2016 volume 533, pages 420-424 disclosed deaminase coupled with CRISPR-Cas9 to create programmable DNA base editors. Since it engages in exogenous editing proteins, it requires the delivery of both the CRISPR/Cas9 system and the guide RNA.

Among other things, the present disclosure provides technologies comprising one or more features such as sugar modifications, base modifications, internucleotidic linkage modifications, control of stereochemistry, various patterns thereof, etc. to solve one or more or all disadvantaged suffered from prior adenosine editing technologies, for example, through providing chirally controlled oligonucleotide compositions of designed oligonucleotides described herein. For example, as demonstrated herein, ADAR-recruiting loops are optional and not required for provided technology.

As appreciated by those skilled in the art, one or more of such useful features may be utilized to improve oligonucleotides in prior technologies (e.g., those described in WO2016097212, WO2017220751, WO2018041973, WO2018134301A1, oligonucleotides and oligonucleotide compositions of each of which are independently incorporated by reference). In some embodiments, the present disclosure provides improvements of prior technologies by apply one or more useful features described herein to prior reported oligonucleotide base sequences. In some embodiments, the present disclosure provides chirally controlled oligonucleotide compositions of previously reported oligonucleotides that may be useful for adenosine editing. In some embodiments, the present disclosure provides improvements of previously reported adenosine editing using stereorandom oligonucleotide compositions by performing such editing using chirally controlled oligonucleotide compositions.

As reported, ADAR proteins may have various isoforms. For example, ADAR1 has, among others, a reported p110 isoform and a reported p150 isoform. In some embodiments, it was observed that certain chirally controlled oligonucleotide compositions can provide high levels of adenosine modification (e.g., conversion of A to I) with multiple isoforms, in some embodiments, both p110 and p150 isoforms, while stereorandom compositions provide low levels of adenosine modification for one or more isoforms (e.g., p110). In some embodiments, chirally controlled oligonucleotide composition are particularly useful for adenosine modification in systems (e.g., cells, tissues, organs, organisms, subjects, etc.) expressing or comprising the p110 isoform of ADAR1, particularly those expressing or comprising high levels of the p110 isoform of ADAR1 relative to the p150 isoform, or those expressing no or low levels of ADAR1 p150.

In some embodiments, the present disclosure provides Cis-acting (CisA) oligonucleotide that do not require stem loop in the structure. In some embodiments, a provided oligonucleotide can form a dsRNA structure with a target mRNA through base pairing. In some embodiments, formed dsRNA structures (optionally with secondary mismatches) contain bulges that promote ADAR binding and therefore, can facilitate ADAR-mediated editing (e.g., deamination of a target qdenosine). In some embodiments, oligonucleotides of the present disclosure are shorter than LSL oligonucleotides or CSL oligonucleotides, e.g., no more than or about 32 nt, no more than or about 31 nt, no more than or about 30 nt, no more than or about 29 nt, no more than or about 28 nt, no more than or about 27 nt, or no more than or about 26 nt in length, and can provide high editing efficiency.

### Assessment/Characterization of Providing Technologies

As appreciated by those skilled in the art, various technologies may be utilized to assess/characterize provided technologies in accordance with the present disclosure. Certain useful technologies are described in the Examples; as demonstrated, among other things, the present disclosure describes various in vivo and in vitro technologies suitable for assessing and characterizing provided technologies. In some embodiments, provided technologies are assessed/characterized, e.g., in cells, with or without exogenous ADAR polypeptides; additionally or alternatively, in some embodiments, provided technologies are assessed/characterized, e.g., in animals, e.g., non-human primates and mice.

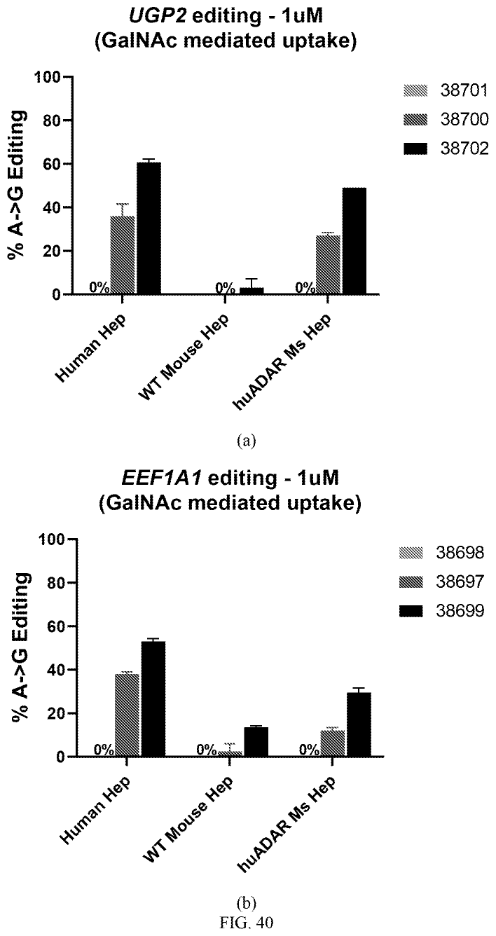

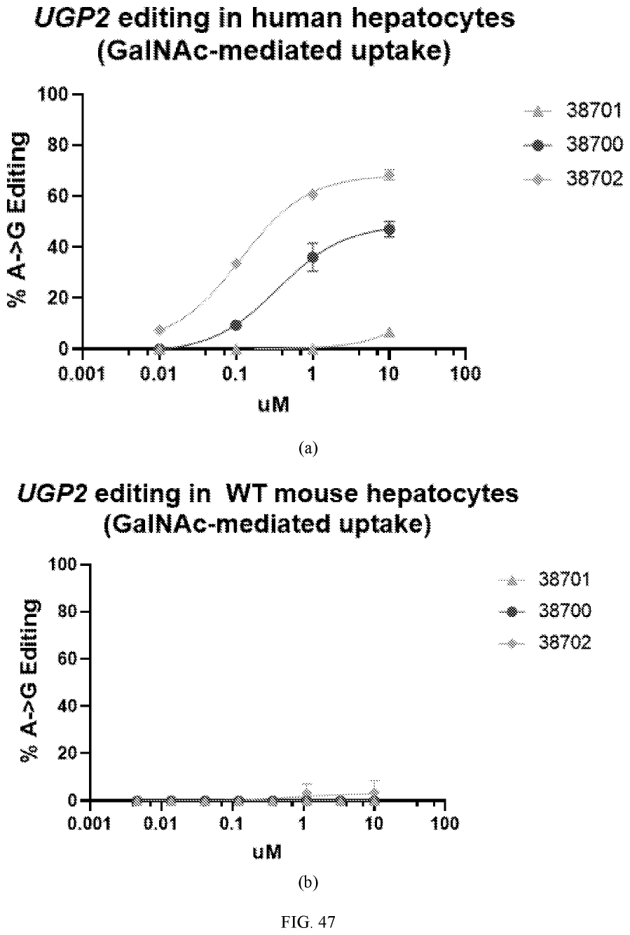

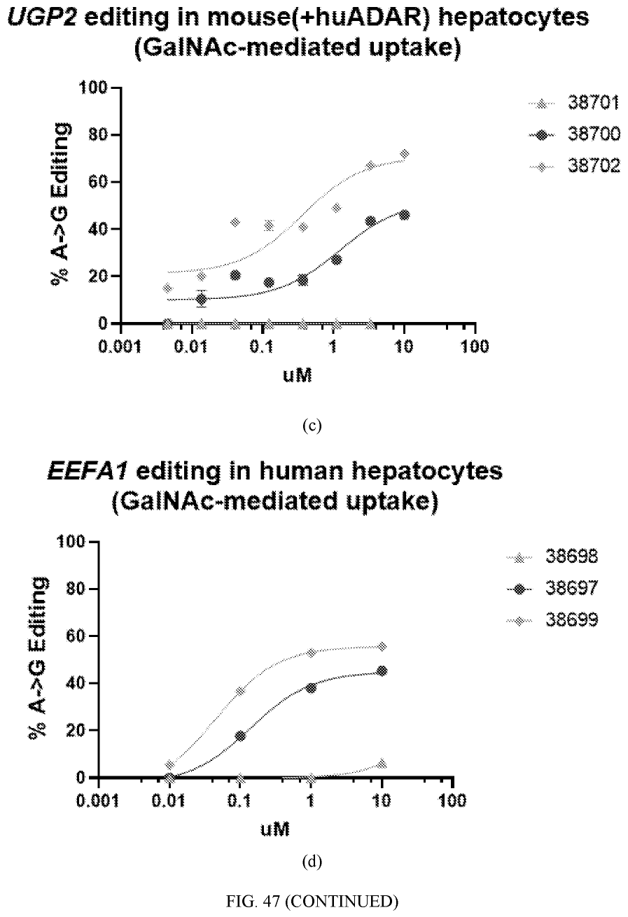

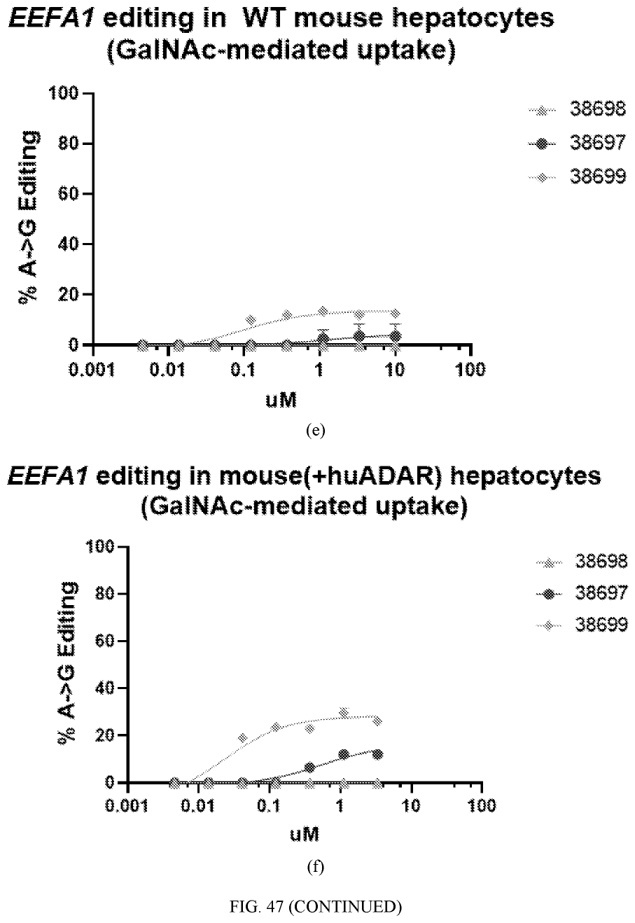

Among other things, the present disclosure encompasses the insights that various agents (e.g., oligonucleotides) and compositions thereof that can provide editing in various human systems, e.g., cells, may show no or much lower levels of editing in certain cells (e.g., mouse cells) and certain animals such as rodents (e.g., mice) that do not contain or express human ADAR, e.g., human ADAR1. Particularly, mice, a commonly used animal model, may be of limited uses for assessing various agents (e.g., oligonucleotides) for editing in humans, as various agents active in human cells provide no or very low levels of activity in mouse cells and animals not engineered to comprise or express a proper ADAR1 (e.g., human ADAR1) polypeptide or a characteristic portion thereof (see FIG. 40 and FIG. 47, data for wild-type (WT) mice and cells, human cells, and cells and mice engineered to express hADAR1 p110 (huADAR mouse)). In some embodiments, the present disclosure provides engineered cells and non-human animals expressing human ADAR1 polypeptide or a characteristic portion thereof. In some embodiments, such cells and human are useful for assessing and characterizing provided technologies. In some embodiments, a human ADAR1 polypeptide or a characteristic portion thereof is or comprises human ADAR1 polypeptide or a characteristic portion thereof. In some embodiments, a human ADAR1 polypeptide or a characteristic portion thereof is or comprises human ADAR1 p110 polypeptide or a characteristic portion thereof. In some embodiments, a human ADAR1 polypeptide or a characteristic portion thereof is or comprises human ADAR1 p150 polypeptide or a characteristic portion thereof. In some embodiments, a human ADAR1 polypeptide or a characteristic portion thereof is or comprises human ADAR1. In some embodiments, a human ADAR1 polypeptide or a characteristic portion thereof is or comprises a human ADAR1 p110 peptide. In some embodiments, a human ADAR1 polypeptide or a characteristic portion thereof is or comprises a human ADAR1 p150 peptide. In some embodiments, a human ADAR1 polypeptide or a characteristic portion thereof is or comprises one or more or all of the following domains of human ADAR1: Z-DNA binding domains, dsRNA binding domains, and deaminase domain. In some embodiments, a human ADAR1 polypeptide or a characteristic portion thereof is or comprises one or both of human ADAR1 Z-DNA binding domains; alternatively or additionally, in some embodiments, a human ADAR1 polypeptide or a characteristic portion thereof is or comprises one, two or all of human ADAR1 dsRNA binding domains; alternatively or additionally, a human ADAR1 polypeptide or a characteristic portion thereof is or comprises a human deaminase domain. In some embodiments, a human ADAR1 polypeptide or a characteristic portion thereof may be expressed together with a mouse ADAR1 polypeptide or a characteristic portion thereof, e.g., one or more human dsRNA binding domains may be engineered to be expressed together with a mouse deaminase domain to form a human-mouse hybrid ADAR1 polypeptide. In some embodiments, cells and/or non-human animals are engineered to comprise and/or express a polynucleotide encoding a human ADAR1 polypeptide or a characteristic portion thereof as described herein. In some embodiments, genomes of cells and/or non-human animals are engineered to comprise a polynucleotide encoding a human ADAR1 polypeptide or a characteristic portion thereof as described herein. In some embodiments, germline genomes of cells and/or non-human animals are engineered to comprise a polynucleotide encoding a human ADAR1 polypeptide or a characteristic portion thereof as described herein. In some embodiments, cells and non-human animals are engineered to comprise, e.g., in their genomes (in some embodiments, germline genomes), one or more A to G mutations each independently associated with a condition, disorder or disease (e.g., a mutation (e.g., c. 1024G>A) in SERPINA1 gene that leads to a glutamate to lysine substitution at amino acid position 342 (E342K) of an AIAT protein). As demonstrated herein, among other things such cells and animals are useful for assessing/characterizing provided technologies, e.g., various oligonucleotides and compositions thereof, e.g., for their editing properties and/or activities, including for their uses against one or more conditions, disorders or diseases. In some embodiments, cells are rodent cells. In some embodiments, cells are mouse cells. In some embodiments, an animal is a rodent. In some embodiments, an animal is a mice.

In some embodiments, the present disclosure provides technologies for assessing/characterizing for assessing cells and/or non-human animals, including those engineered to comprise or express an ADAR1 polypeptide or a characteristic portion thereof, or a polynucleotide encoding an ADAR1 polypeptide or a characteristic portion thereof, which ADAR1 polypeptide or a characteristic portion thereof and/or polynucleotide is not in and/or is not expressed in the cells and/or non-human animals prior to engineering. In some embodiments, a provided method comprises administering to a cell or a population thereof one or more oligonucleotides or compositions which one or more oligonucleotides or compositions can each independently edit an adenosine in a comparable human cell or a population thereof. In some embodiments, a provided method comprises administering to an animal or a population thereof one or more oligonucleotides or compositions which one or more oligonucleotides or compositions can each independently edit an adenosine in a human cell or a population thereof. In some embodiments, editing levels in cells to be assessed/characterized, or in cells from animals, are compared to those observed in comparable human cells. In some embodiments, comparable human cells are of the same type as cells to be assessed/characterized or cells from animals. In some embodiments, cells are rodent cells. In some embodiments, cells are mouse cells. In some embodiments, an animal is a rodent. In some embodiments, an animal is a mice. In some embodiments, one or more oligonucleotides or compositions are administered separately to separate cells and/or animals. In some embodiments, one or more oligonucleotides or compositions may be administered to the same collection of cells and/or animals, optionally simultaneously. Various oligonucleotides and compositions that can edit various target adenosines are as described herein and can be utilized accordingly.

### Uses and Applications

As appreciated by those skilled in the art, oligonucleotides are useful for multiple purposes. In some embodiments, provided technologies (e.g., oligonucleotides, compositions, methods, etc.) can be useful for modulating levels and/or activities of various nucleic acids (e.g., RNA) and/or products encoded thereby (e.g., proteins). In some embodiments, provided technologies can reduce levels and/or activities of undesired target nucleic acids (e.g., comprising undesired adenosine) and/or products thereof. In some embodiments, provided technologies can increase levels and/or activities of desired target nucleic acids (e.g., comprising I instead of undesired adenosine at one or more locations) and/or products thereof.

For example, in some embodiments, provided technologies can be utilized as single-stranded oligonucleotides for site-directed editing of target adenosine in target RNA sequences. In some embodiments, provided technologies are capable of modulating levels of expressions and activities. Among other things, the present disclosure provides improvement by provided technologies which can be improvement of various desired biological functions, including but not limited to treatment and/or prevention of various conditions, disorders or diseases (e.g., those associated with G to A mutation).

In some embodiments, provided technologies can modulate activities and/or functions of a target gene. In some embodiments, a target gene is a gene with respect to which expression and/or activity of one or more gene products (e.g., RNA and/or protein products) are intended to be altered. In many embodiments, target genes have target adenosine residues to be altered and can benefit from conversion of such residues to inosine residues. In some embodiments, when an oligonucleotide as described herein acts on a particular target gene, level and/or activity of one or more gene products of that gene can be altered when the oligonucleotide is present as compared with when it is absent.

In some embodiments, provided oligonucleotides and compositions are useful for treating various conditions, disorders, or diseases, by reducing levels and/or activities of target transcripts and/or products encoded thereby that are associated with the conditions, disorders, or diseases, and optionally providing transcripts and/or products encoded thereby that are less associated or not associated with the conditions, disorders or diseases (e.g., by conversion of target adenosine to inosine to correct G to A mutations, to alter splicing, etc.). In some embodiments, the present disclosure provides methods for preventing or treating a condition, disorder, or disease, comprising administering to a subject susceptible thereto or suffering therefrom an effective amount of a provided oligonucleotide or composition. In some embodiments, the present disclosure provides methods for preventing or treating a condition, disorder, or disease, comprising administering to a subject susceptible to or suffering from a condition, disorder or disease a provided single-stranded oligonucleotide for site-directed editing of a nucleotide (e.g. target adenosine) in a target RNA sequence, or a composition thereof. In some embodiments, a provided single-stranded oligonucleotide for site-directed editing of a nucleotide in a target RNA sequence is of a base sequence that partially or fully complementary to a portion of a transcript, which transcript is associated with a condition, disorder, or disease. In some embodiments, a base sequence is such that it preferentially binds to a transcript associated with a condition, disorder or disease over other transcripts that are not associated with said condition, disorder, or disease. In some embodiments, a condition, disorder, or disease is associated with a G to A mutation.

In some embodiments, oligonucleotide compositions in provided methods are chirally controlled oligonucleotide compositions. In some embodiments, a method of treating a condition, disorder or disease can include administering a composition comprising a plurality of oligonucleotides sharing a common base sequence, which base sequence is complementary to a target sequence in a target transcript. Among other things, the present disclosure provides an improvement that comprises administering as the oligonucleotide composition a chirally controlled oligonucleotide composition as described in the present disclosure, characterized in that, when it is contacted with the target transcript in a system, adenosine editing of the transcript is improved relative to that observed under a reference condition selected from the group consisting of absence of the composition, presence of a reference composition, and any combinations thereof. In some embodiments, a reference composition is a racemic preparation of oligonucleotides of the same sequence or constitution. In some embodiments, a target transcript is an oligonucleotide transcript.

As appreciated by those skilled in the art, among other things, provided technologies can be utilized for various applications which involve and/or can benefit from an adenosine to inosine conversion. Certain applications are described in below.

| | | Treatment Modality | | |
|---|---|---|---|---|
| | | | oligonucleotide/ | |
| | | oligonucleotide- | siRNA-mediated | |
| Application | Application | mediated splicing | silencing | RNA editing |
| Alter mRNA splicing | Exon | ✓ | | ✓ |
| | skipping/inclusion/ | | | |
| | restore frame | | | |
| Silence protein | Reduce levels of toxic | | ✓ | ✓ |
| expression | mRNA/protein | | | |
| Fix nonsense mutations | Restore protein | | | ✓ |
| (e.g. those that cannot be | expression | | | |
| splice-corrected) | | | | |
| Fix missense mutations | Restore protein function | | | ✓ |
| (e.g., those that cannot | | | | |
| be splice-corrected) | | | | |
| Modify amino acid | Alter protein | | | ✓ |
| codons | level/function | | | |
| Remove upstream ORF | Increase protein | | | ✓ |
| | expression | | | |

Those skilled in the art reading the present disclosure will appreciate that various G to A mutations, e.g., those in transcripts from C to T mutations, a type of the most common mutations occurring in human genes, may be corrected and thus benefit from provided technologies. In some embodiments, provided technologies may be utilized to target mutations associated with various polar or charged amino acids (e.g., Ser, Tyr, Asp, Glu, His, Asn, Gln, Lys, etc.), stop codons (opal, ochre and amber), transcriptional start sites, splicing signals, microRNA recognition sites, repetitive elements, microRNAs (miRNAs), protein encoding transcripts, etc. Among other things, provided technologies can elicit diverse functional outcomes, e.g., altered splicing, restored/improved protein expression and/or functions, etc.

In some embodiments, through editing provided technology can restore protein functions (e.g., fix nonsense and missense mutations that cannot be splice-corrected, remove stop mutations, prevent protein misfolding and aggregation, etc., and can be utilized for preventing and/or treating various conditions, disorders or diseases such as recessive or dominant genetically defined diseases), modify protein functions (e.g., alter protein processing (e.g., protease cleavage sites), protein-protein interactions, modulate signaling pathways, etc., and can be utilized for preventing and/or treating various conditions, disorders or diseases such as those related to ion channel permeability), protein upregulation (e.g., miRNA target site modification, modifying upstream ORFs, Modification of ubiquitination sites, etc., and can be utilized for preventing and/or treating various conditions, disorders or diseases such as Haploinsufficient diseases)).

Certain applications are described, e.g., in WO2016097212, WO2017220751, WO2018041973, and/or WO2018134301A1.

In some embodiments, when an oligonucleotide or oligonucleotide composition is contacted with a target nucleic acid comprising a target adenosine in a system, a target adenosine in a target nucleic acid is modified. In some embodiments, when an oligonucleotide or oligonucleotide composition is contacted with a target nucleic acid comprising a target adenosine in a system, level of a target nucleic acid is reduced compared to absence of the product or presence of a reference oligonucleotide. In some embodiments, when an oligonucleotide or oligonucleotide composition is contacted with a target nucleic acid comprising a target adenosine in a system, splicing of a target nucleic acid or a product thereof is altered compared to absence of the oligonucleotide or presence of a reference oligonucleotide. In some embodiments, when an oligonucleotide or oligonucleotide composition is contacted with a target nucleic acid comprising a target adenosine in a system, level of a product of a target nucleic acid is altered compared to absence of the product or presence of a reference oligonucleotide. In some embodiments, level of a product is increased, wherein the product is or is encoded by a nucleic acid which is otherwise identical to a target nucleic acid but a target adenosine is modified. In some embodiments, level of a product is increased, wherein the product is or is encoded by a nucleic acid which is otherwise identical to a target nucleic acid but a target adenosine is replaced with inosine. In some embodiments, level of a product is increased, wherein the product is or is encoded by a nucleic acid which is otherwise identical to a target nucleic acid but the adenine of a target adenosine is replaced with guanine. In some embodiments, a product is a protein. In some embodiments, a target adenosine is a mutation from guanine. In some embodiments, a target adenosine is more associated with a condition, disorder or disease than a guanine at the same position. In some embodiments, an oligonucleotide is capable of forming a double-stranded complex with a target nucleic acid. In some embodiments, a target nucleic acid or a portion thereof is or comprises RNA. In some embodiments, a target adenosine is of an RNA. In some embodiments, a target adenosine is modified, and the modification is or comprises deamination of a target adenosine. In some embodiments, a target adenosine is modified and the modification is or comprises conversion of a target adenosine to an inosine. In some embodiments, a modification is promoted by an ADAR protein. In some embodiments, a system is an in vitro or ex vivo system comprising an ADAR protein. In some embodiments, a system is or comprises a cell that comprises or expresses an ADAR protein. In some embodiments, a system is a subject comprising a cell that comprises or expresses an ADAR protein. In some embodiments, a ADAR protein is ADAR1. In some embodiments, an ADAR1 protein is or comprises p110 isoform. In some embodiments, an ADAR1 protein is or comprises p150 isoform. In some embodiments, an ADAR1 protein is or comprises p110 and p150 isoform. In some embodiments, a ADAR protein is ADAR2. As demonstrated herein, the present disclosure among other things provides technologies for recruiting enzymes to target sites (e.g., those comprising target As), comprising contacting such target sites with, or administering to systems comprising or expressing polynucleotide (e.g., RNA) comprising such target sites, provided oligonucleotides or compositions thereof. In some embodiments, an enzyme is an RNA-editing enzyme such as ADAR1, ADAR2, etc. as described herein.

In some embodiments, an oligonucleotide composition comprising a plurality of oligonucleotides provide a greater level, e.g., a target adenosine is modified at a greater level, than that is observed with a comparable reference oligonucleotide composition. In some embodiments, a reference oligonucleotide composition comprises no or a lower level of oligonucleotides of the plurality. In some embodiments, a reference composition does not contain oligonucleotides that have the same constitution as an oligonucleotide of the plurality. In some embodiments, a reference composition does not contain oligonucleotides that have the same structure as an oligonucleotide of the plurality. In some embodiments, a reference oligonucleotide composition is a composition whose oligonucleotides having the same base sequence as oligonucleotides of the plurality contain a lower level of 2′-F modifications compared to oligonucleotides of the plurality. In some embodiments, a reference oligonucleotide composition is a composition whose oligonucleotides having the same base sequence as oligonucleotides of the plurality contain a lower level of 2′-OMe modifications compared to oligonucleotides of the plurality. In some embodiments, a reference oligonucleotide composition is a composition whose oligonucleotides having the same base sequence as oligonucleotides of the plurality have a different sugar modification pattern compared to oligonucleotides of the plurality. In some embodiments, a reference oligonucleotide composition is a composition whose oligonucleotides having the same base sequence as oligonucleotides of the plurality contain a lower level of modified internucleotidic linkages compared to oligonucleotides of the plurality. In some embodiments, a reference oligonucleotide composition is a composition whose oligonucleotides having the same base sequence as oligonucleotides of the plurality contain a lower level of phosphorothioate internucleotidic linkages compared to oligonucleotides of the plurality. In some embodiments, a composition is a stereorandom oligonucleotide composition. In some embodiments, a reference composition is a stereorandom oligonucleotide composition of oligonucleotides of the same constitution as oligonucleotides of the plurality.

In some embodiments, the present disclosure provides technologies for modifying a target adenosine in a target nucleic acid, comprising contacting a target nucleic acid with an provided oligonucleotide or oligonucleotide composition as described herein. In some embodiments, the present disclosure provides a method for deaminating a target adenosine in a target nucleic acid, comprising contacting a target nucleic acid with an oligonucleotide or composition as described herein. In some embodiments, the present disclosure provides a method for producing, or restoring or increasing level of a product of a particular nucleic acid, comprising contacting a target nucleic acid with a povided oligonucleotide or composition wherein a target nucleic acid comprises a target adenosine, and the particular nucleic acid differs from a target nucleic acid in that the particular nucleic acid has an I or G instead of a target adenosine. In some embodiments, the present disclosure provides a method for reducing level of a product of a target nucleic acid, comprising contacting a target nucleic acid with an oligonucleotide or composition of the present disclosure, wherein a target nucleic acid comprises a target adenosine. In some embodiments, a product is a protein. In some embodiments, a product is a mRNA.

In some embodiments, the present disclosure provides a method, comprising:

contacting an oligonucleotide or composition with a sample comprising a target nucleic acid and an adenosine deaminase, wherein:

the base sequence of the oligonucleotide or oligonucleotides in the oligonucleotide composition is substantially complementary to that of a target nucleic acid; and

a target nucleic acid comprises a target adenosine;

wherein a target adenosine is modified.

In some embodiments, the present disclosure provides a method comprising:

1) obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and

2) obtaining a reference level of modification of a target adenosine in a target nucleic acid, which level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;

wherein:

oligonucleotides of the first plurality comprise more sugars with 2′-F modification, more sugars with 2′-OR modification wherein R is not —H, and/or more chiral internucleotidic linkages than oligonucleotides of the reference plurality; and

the first oligonucleotide composition provides a higher level of modification compared to oligonucleotides of the reference oligonucleotide composition.

In some embodiments, the present disclosure provides a method comprising:

obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and

wherein the first level of modification of a target adenosine is higher than a reference level of modification of a target adenosine, wherein the reference level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;

wherein:

oligonucleotides of the first plurality comprise more sugars with 2′-F modification, more sugars with 2′-OR modification wherein R is not —H, and/or more chiral internucleotidic linkages than oligonucleotides of the reference plurality.

In some embodiments, the present disclosure provides a method comprising:

1) obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and

2) obtaining a reference level of modification of a target adenosine in a target nucleic acid, which level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;

wherein:

oligonucleotides of the first plurality comprise more sugars with 2′-F modification, more sugars with 2′-OR modification wherein R is not —H, and/or more chirally controlled chiral internucleotidic linkages than oligonucleotides of the reference plurality; and

the first oligonucleotide composition provides a higher level of modification compared to oligonucleotides of the reference oligonucleotide composition.

In some embodiments, the present disclosure provides a method comprising:

obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and

wherein the first level of modification of a target adenosine is higher than a reference level of modification of a target adenosine, wherein the reference level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;

wherein:

oligonucleotides of the first plurality comprise more sugars with 2′-F modification, more sugars with 2′-OR modification wherein R is not —H, and/or more chirally controlled chiral internucleotidic linkages than oligonucleotides of the reference plurality.

In some embodiments, the present disclosure provides a method comprising:

1) obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and

2) obtaining a reference level of modification of a target adenosine in a target nucleic acid, which level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;

wherein:

oligonucleotides of the first plurality comprise one or more chirally controlled chiral internucleotidic linkages; and

oligonucleotides of the reference plurality comprise no chirally controlled chiral internucleotidic linkages (a reference oligonucleotide composition is a “stereorandom composition); and

the first oligonucleotide composition provides a higher level of modification compared to oligonucleotides of the reference oligonucleotide composition.

In some embodiments, the present disclosure provides a method comprising:

obtaining a first level of modification of a target adenosine in a target nucleic acid, which level is observed when a first oligonucleotide composition is contacted with a sample comprising a target nucleic acid and an adenosine deaminase, wherein the first oligonucleotide composition comprises a first plurality of oligonucleotides sharing the same base sequence which is substantially complementary to that of a target nucleic acid; and

wherein the first level of modification of a target adenosine is higher than a reference level of modification of a target adenosine, wherein the reference level is observed when a reference oligonucleotide composition is contacted with a sample comprising a target nucleic acid and an adenosine deaminase, wherein the reference oligonucleotide composition comprises a reference plurality of oligonucleotides sharing the same base sequence which is substantially complementary to that of a target nucleic acid;

wherein:

oligonucleotides of the first plurality comprise one or more chirally controlled chiral internucleotidic linkages; and

oligonucleotides of the reference plurality comprise no chirally controlled chiral internucleotidic linkages (a reference oligonucleotide composition is a “stereorandom composition).

In some embodiments, a first oligonucleotide composition is an oligonucleotide composition as described herein. In some embodiments, a first oligonucleotide composition is a chirally controlled oligonucleotide composition. In some embodiments, a deaminase is an ADAR enzyme. In some embodiments, a deaminase is ADAR1. In some embodiments, a deaminase is ADAR2. In some embodiments, a sample is or comprise a cell. In some embodiments, a target nucleic acid is more associated with a condition, disorder or disease, or decrease of a desired property or function, or increase of an undesired property or function, compared to a nucleic acid which differs from a target nucleic acid in that it has an I or G at the position of a target adenosine instead of a target adenosine. In some embodiments, a target adenosine is a G to A mutation.

Among other things, oligonucleotide designs of the present disclosure, e.g., nucleobase, sugar, internucleotidic linkage modifications, control of linkage phosphorus stereochemistry, and/or patterns thereof, can be applied to improve prior technologies. In some embodiments, the present disclosure provides improvement over prior technologies by introducing one or more structural features of the present disclosure, e.g., nucleobase, sugar, internucleotidic linkage modifications, control of linkage phosphorus stereochemistry, and/or patterns thereof to oligonucleotides in prior technologies. In some embodiments, an improvement is or comprises improvement from control of linkage phosphorus stereochemistry.

In some embodiments, a provided oligonucleotide or oligonucleotide composition does not cause significant degradation of a nucleic acid (e.g., no more than about 5%-100% (e.g., no more than about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.)). In some embodiments, a composition does not cause significant undesired exon skipping or altered exon inclusion in a target nucleic acid (e.g., no more than about 5%-100% (e.g., no more than about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.)).

In some embodiments, provided technologies can provide high levels of adenosine editing (e.g., conversion to inosine). In some embodiments, percentage of target adenosine editing is about 10%-100%, e.g., at least about 10%, 15%, 20%, 25%, 30%, 35%, 40%, 45%, 50%, 60%, 70%, 75%, 80%, 85%, 90%, or 95%. In some embodiments, it is at least 10%. In some embodiments, it is at least 15%. In some embodiments, it is at least 20%. In some embodiments, it is at least 25%. In some embodiments, it is at least 30%. In some embodiments, it is at least 35%. In some embodiments, it is at least 40%. In some embodiments, it is at least 45%. In some embodiments, it is at least 50%. In some embodiments, it is at least 60%. In some embodiments, it is at least 70%. In some embodiments, it is at least 75%. In some embodiments, it is at least 80%. In some embodiments, it is at least 85%. In some embodiments, it is at least 90%. In some embodiments, it is at least 95%. In some embodiments, it is at least about 100%.

In some embodiments, an oligonucleotide or a composition thereof is capable of mediating a decrease in the expression or level of a target nucleic acid or a product thereof (e.g., by modifying a target adenosine into inosine). In some embodiments, an oligonucleotide or a composition thereof is capable of mediating a decrease in the expression or level of a target gene or a gene product thereof (e.g., by modifying a target adenosine into inosine) in a cell in vitro. In some embodiments, expression or level can be decreased by at least about 10%, 15%, 20%, 25%, 30%, 35%, 40%, 45%, 50%, 60%, 70%, 75%, 80%, 85%, 90%, or 95%. In some embodiments, expression or level of a target gene or a gene product thereof can be decreased by at least about 10%, 15%, 20%, 25%, 30%, 35%, 40%, 45%, 50%, 60%, 70%, 75%, 80%, 85%, 90%, or 95% by ADAR-mediated deamination directed by an oligonucleotide or a composition thereof, e.g., at a concentration of 10 uM or less in a cell(s) in vitro. In some embodiments, an oligonucleotide or a composition thereof is capable of provide suitable levels of activities at a concentration of 1 nM, 5 nM, 10 nM or less (e.g., when assayed in cells in vitro or in vivo).

In some embodiments, activity of provided oligonucleotides and compositions may be assessed by IC50, which is the inhibitory concentration to decrease level of a target nucleic acid or a product thereof by 50% in a suitable condition, e.g., cell-based in vitro assays. In some embodiments, provided oligonucleotides or compositions have an IC50 no more than 0.001, 0.01, 0.1, 0.5, 1, 2, 5, 10, 50, 100, 200, 500 or 1000 nM, e.g., when assessed in cell-based assays. In some embodiments, an IC50 is no more than about 500 nM. In some embodiments, an IC50 is no more than about 200 nM. In some embodiments, an IC50 is no more than about 100 nM. In some embodiments, an IC50 is no more than about 50 nM. In some embodiments, an IC50 is no more than about 25 nM. In some embodiments, an IC50 is no more than about 10 nM. In some embodiments, an IC50 is no more than about 5 nM. In some embodiments, an IC50 is no more than about 2 nM. In some embodiments, an IC50 is no more than about 1 nM. In some embodiments, an IC50 is no more than about 0.5 nM.

In some embodiments, provided technologies can provide selective editing of target adenosine over other adenosine residues in a target adenosine. In some embodiments, selectivity of a target adenosine over a non-target adenosine is at least 1.5, 2, 3, 4, 5, 6, 7, 8, 9, 10, 15, 20, 25, 30, 40, 50, 60, 70, 80, 90, 100 fold or more (e.g., as measured by level of editing of a target adenosine over a non-target adenosine at a suitable condition, or by oligonucleotide concentrations for a certain level of editing (e.g., 0.5%, 1%, 2%, 5%, 10%, 15%, 20%, 25%, 30%, 40%, 50%, etc.). In some embodiments, a selectivity is at least 2 fold. In some embodiments, a selectivity is at least 3 fold. In some embodiments, a selectivity is at least 4 fold. In some embodiments, a selectivity is at least 5 fold. In some embodiments, a selectivity is at least 10 fold. In some embodiments, a selectivity is at least 25 fold. In some embodiments, a selectivity is at least 50 fold. In some embodiments, a selectivity is at least 100 fold.

In some embodiments, the present disclosure provides a method for suppression of a transcript from a target nucleic acid sequence for which one or more similar nucleic acid sequences exist within a population, each of the target and similar sequences contains a specific characteristic sequence element that defines the target sequence relative to the similar sequences, the method comprising contacting a sample comprising transcripts of target nucleic acid sequence with an oligonucleotide, or a composition comprising a plurality of oligonucleotides sharing a common base sequence, wherein the base sequence of the oligonucleotide, or the common base sequence of the plurality of oligonucleotide, is or comprises a sequence that is complementary to the characteristic sequence element that defines the target nucleic acid sequence. In some embodiments, wherein when the oligonucleotide, or the oligonucleotide composition, is contacted with a system comprising transcripts of both the target nucleic acid sequence and a similar nucleic acid sequences, transcripts of the target nucleic acid sequence are suppressed at a greater level than a level of suppression observed for a similar nucleic acid sequence. In some embodiments, suppression of the transcripts of the target nucleic acid sequence can be 1.1-100, 2-100, 1.5, 2, 2.5, 3, 4, 5, 6, 7, 8, 9, or 10-fold greater than suppression observed for a similar nucleic acid sequence. In some embodiments, a target nucleic acid sequence is associated with (or more associated with compared to a similar nucleic acid sequence) a condition, disorder or disease. As those skilled in the art will appreciate, selective reduction of a transcript (and/or products thereof) associated with conditions, disorders or diseases, while maintaining transcripts that are not, or are less, associated with conditions, disorders or diseases can provide a number of advantages, for example, providing disease treatment and/or prevention while maintaining one or more desired biological functions (which may provide, among other things, fewer or less severe side effects).

In some embodiments, as demonstrated herein, selectivity is at least 10 fold, or 20, 30, 40, or 50 fold or more in a system, e.g. a reporter assay described herein. In some embodiments, an oligonucleotide or composition can effectively reduce levels of mutant protein (e.g., at least 50%, 60%, 70% or more reduction of a mutant protein) while maintaining levels of wild-type protein (e.g. at least 70%, 75%, 80%, 85%, 90%, 95%, or more wild-type protein remaining) in a system. In some embodiments, provided oligonucleotides are stable in various biological systems, e.g. in mouse brain homogenates (e.g., at least 70%, 75%, 80%, 85%, 90%, 95%, or more remaining after 1, 2, 3, 4, 5, 6, 7, or 8 days). In some embodiments, provided oligonucleotides are of low toxicity. In some embodiments, provided oligonucleotides and compositions thereof, e.g., chirally controlled oligonucleotides and compositions thereof, do not significant activate TLR9 (e.g., when compared to reference oligonucleotides and compositions thereof (e.g., corresponding stereorandom oligonucleotides and compositions thereof)). In some embodiments, provided oligonucleotides and compositions thereof, e.g., chirally controlled oligonucleotides and compositions thereof, do not significantly induce complement activation (e.g., when compared to reference oligonucleotides and compositions thereof (e.g., corresponding stereorandom oligonucleotides and compositions thereof)).

For various applications, provided oligonucleotides and/or compositions may be provided as pharmaceutical compositions. In some embodiments, the present disclosure provides a pharmaceutical composition which comprises or delivers an effective amount of an oligonucleotide or a pharmaceutically acceptable salt thereof. In some embodiments, a pharmaceutical composition may comprise various forms of an oligonucleotide, e.g., acid, base and various pharmaceutically acceptable salt forms. In some embodiments, a pharmaceutically acceptable salt is sodium salt. In some embodiments, a pharmaceutically acceptable salt is a potassium salt. In some embodiments, a pharmaceutically acceptable salt is a amine salt (e.g., of an amine having the structure of N(R)₃). In some embodiments, a pharmaceutical composition further comprises a pharmaceutically acceptable carrier. In some embodiments, a pharmaceutical composition is or comprises a liquid solution. In some embodiments, a liquid composition has a controlled pH range, e.g., around or being physiological pH.

Among other things, the present disclosure provides technologies for preventing or treating conditions, disorders or diseases. In some embodiments, a condition, disorder or disease is amenable to (e.g., can benefit from) A to I conversion. In some embodiments, the present disclosure provides a method for preventing or treating a condition, disorder or disease amenable to a G to A mutation, comprising administering to a subject susceptible thereto or suffering therefrom an effective amount of an oligonucleotide or composition as described herein. In some embodiments, the present disclosure provides a method for preventing or treating a condition, disorder or disease associated with a G to A mutation, comprising administering to a subject susceptible thereto or suffering therefrom an effective amount of an oligonucleotide or composition as described herein. In some embodiments, the base sequence of the oligonucleotide or oligonucleotides in the oligonucleotide composition is substantially complementary to that of the target nucleic acid comprising a target adenosine. In some embodiments, cells, tissues or organs associated with the condition, disorder or disease comprise or express an ADAR protein. In some embodiments, cells, tissues or organs associated with the condition, disorder or disease comprise or express ADAR1 (e.g., a p110 and/or a p150 forms). In some embodiments, cells, tissues or organs associated with the condition, disorder or disease comprise or express ADAR2. In some embodiments, a condition, disorder or disease is as described herein. In some embodiments, a condition, disorder or disease is Alpha-1 antitrypsin deficiency. In some embodiments, a method comprises converting a target adenosine to I.

In some embodiments, the present disclosure provides an oligonucleotide comprising a sequence complementary to a target sequence. In some embodiments, the present disclosure provides an oligonucleotide which directs site-specific (can also be referred as site directed) editing (e.g., deamination). In some embodiments, the present disclosure provides an oligonucleotide which directs site-specific adenosine editing mediated by ADAR (e.g., an endogenous ADAR). Various provided oligonucleotides can be utilized as single-stranded oligonucleotides for site-directed editing of a nucleotide in a target RNA sequence. In some embodiments, the present disclosure provides methods for preventing and/or treating conditions, disorders, or diseases associated with a G to A mutation in a target sequence using provided single-stranded oligonucleotides for site-directed editing of a nucleotide in a target RNA sequence and compositions thereof. In some embodiments, the present disclosure provides oligonucleotides and compositions thereof for use as medicaments, e.g., for conditions, disorders, or diseases associated with a G to A mutation in a target sequence. In some embodiments, the present disclosure provides oligonucleotides and compositions thereof for use in the treatment of conditions, disorders or diseases associated with a G to A mutation in a target sequence. In some embodiments, the present disclosure provides oligonucleotides and compositions thereof for the manufacture of medicaments for the treatment of a related conditions, disorders or diseases associated with a G to A mutation in a target sequence.

In some embodiments, the present disclosure provides a method for preventing, treating or ameliorating a condition, disorder or disease associated with a G to A mutation in a target sequence in a subject susceptible thereto or suffering therefrom, comprising administering to the subject a therapeutically effective amount of an oligonucleotide or a pharmaceutical composition thereof.

In some embodiments, the present disclosure provides a method for deaminating a target adenosine in a target sequence in a cell, comprising: contacting the cell with an oligonucleotide or a composition thereof. In some embodiments, the present disclosure provides a method deaminating a target adenosine in a target sequence (e.g., a transcript) in a cell, comprising: contacting the cell with an oligonucleotide or a composition thereof. In some embodiments, the present disclosure provides a method for reducing the level of a protein associated with a G to A mutation in a cell, comprising: contacting the cell with an oligonucleotide or a composition thereof. In some embodiments, provided methods can selectively reduce levels of a transcripts and/or products encoded thereby that are related to conditions, disorders or diseases associated with a G to A mutation. In some embodiments, provided methods can selectively edit target nucleic acids, e.g., transcripts comprising an undesired A (e.g., a G to A mutation) over otherwise identical nucleic acids which have G at positions of target A.

In some embodiments, the present disclosure provides a method for decreasing a mutated gene (e.g., a G to A mutation) expression in a mammal in need thereof, comprising administering to the mammal a nucleic acid-lipid particle comprising a provided single-stranded oligonucleotide for site-directed editing of a nucleotide in a target RNA sequence or a composition thereof.

In some embodiments, the present disclosure provides a method for in vivo delivery of an oligonucleotide, comprising administering to a mammal an oligonucleotide or a composition thereof.

In some embodiments, a subject or patient suitable for treatment of a condition, disorder, or disease associated with a G to A mutation, can be identified or diagnosed by a health care professional.

In some embodiments, a symptom of a condition, disorder or disease associated with a G to A mutation can be any condition, disorder or disease that can benefit from an A to I conversion.

In some embodiments, a provided single-stranded oligonucleotide for site-directed editing of a nucleotide in a target RNA sequence or a composition thereof can prevent, treat, ameliorate, or slow progression of a condition, disorder or disease associated with a G to A mutation, or at least one symptom of a condition, disorder or disease associated with a G to A mutation.

In some embodiments, a method of the present disclosure can be for the treatment of a condition, disorder or disease associated with a G to A mutation in a subject wherein the method comprises administering to a subject a therapeutically effective amount of an oligonucleotide or a pharmaceutical composition thereof.

In some embodiments, a provided method can reduce at least one symptom of a condition, disorder or disease associated with a G to A mutation wherein the method comprises administering to a subject a therapeutically effective amount of an oligonucleotide or a pharmaceutical composition thereof.

In some embodiments, administration of an oligonucleotide to a patient or subject can be capable of mediating any one or more of: slowing the progression of a condition, disorder or disease associated with a G to A mutation; delaying the onset of a condition, disorder or disease associated with a G to A mutation or at least one symptom thereof; improving one or more indicators of a condition, disorder or disease associated with a G to A mutation; and/or increasing the survival time or lifespan of the patient or subject.

In some embodiments, slowing disease progression can relate to the prevention of, or delay in, a clinically undesirable change in one or more clinical parameters in an individual susceptible to or suffering from a condition, disorder, or disease associated with a G to A mutation, such as those described herein. It is well within the abilities of a physician to identify a slowing of disease progression in an individual susceptible to or suffering a condition, disorder, or disease associated with a G to A mutation, using one or more of the disease assessment tests described herein. Additionally, it is understood that a physician may administer to the individual diagnostic tests other than those described herein to assess the rate of disease progression in an individual susceptible to or suffering from a condition, disorder, or disease associated with a G to A mutation.

A physician may use family history of a condition, disorder, or disease associated with a G to A mutation or comparisons to other patients with similar genetic profile.

In some embodiments, indicators of a condition, disorder, or disease associated with a G to A mutation include parameters employed by a medical professional, such as a physician, to diagnose or measure the progression of the condition, disorder, or disease.

In some embodiments, a subject is administered an oligonucleotide or a composition thereof and an additional agent and/or method, e.g., an additional therapeutic agent and/or method. In some embodiments, an oligonucleotide or composition thereof can be administered alone or in combination with one or more additional therapeutic agents and/or treatment. When administered in combination each component may be administered at the same time or sequentially in any order at different points in time. In some embodiments, each component may be administered separately but sufficiently closely in time so as to provide the desired therapeutic effect. In some embodiments, provided oligonucleotides and additional therapeutic components are administered concurrently. In some embodiments, provided oligonucleotides and additional therapeutic components can be administered as one composition. In some embodiments, at a time point a subject being administered can be exposed to both provided oligonucleotides and additional components at the same time.

In some embodiments, an additional therapeutic agent can be physically conjugated to an oligonucleotide. In some embodiments, an additional agent is GalNAc. In some embodiments, a provided single-stranded oligonucleotide for site-directed editing of a nucleotide in a target RNA sequence can be physically conjugated with an additional agent. In some embodiments, additional agent oligonucleotides can have base sequences, sugars, nucleobases, internucleotidic linkages, patterns of sugar, nucleobase, and/or internucleotidic linkage modifications, patterns of backbone chiral centers, etc., or any combinations thereof, as described in the present disclosure, wherein each T may be independently replaced with U and vice versa. In some embodiments, an oligonucleotide can be physically conjugated to a second oligonucleotide which can decrease (directly or indirectly) the expression, activity, and/or level of a target sequence, or which is useful for treating a condition, disorder, or disease associated with a G to A mutation.

In some embodiments, a provided single-stranded oligonucleotide for site-directed editing of a nucleotide in a target RNA sequence may be administered with one or more additional (or second) therapeutic agent for a condition, disorder or disease associated with a G to A mutation.

In some embodiments, a subject can be administered an oligonucleotide and an additional therapeutic agent, wherein the additional therapeutic agent is an agent described herein or known in the art which is useful for treatment of a condition, disorder or disease to be treated.

In some embodiments, provided single-stranded oligonucleotide for site-directed editing of a nucleotide in a target RNA sequence can be co-administered or be used as part of a treatment regimen along with one or more treatment for a condition, disorder or disease or a symptom thereof, including but not limited to: aptamers, lncRNAs, lncRNA inhibitors, antibodies, peptides, small molecules, other oligonucleotides to a target other targets.

In some embodiments, an additional therapeutic treatment is, as a non-limiting example, a method of editing a gene

In some embodiments, an additional therapeutic agent is, as a non-limiting example, an oligonucleotide.

In some embodiments, a second or additional therapeutic agent can be administered to a subject prior, simultaneously with, or after an oligonucleotide. In some embodiments, a second or additional therapeutic agent can be administered multiple times to a subject, and an oligonucleotide is also administered multiple times to a subject, and the administrations are in any order.

In some embodiments, an improvement may include decreasing the expression, activity and/or level of a gene or gene product which is too high in a disease state; increasing the expression, activity and/or level of a gene or gene product which is too low in the disease state; and/or decreasing the expression, activity and/or level of a mutant and/or disease-associated variant of a gene or gene product.

In some embodiments, an oligonucleotide or composition useful for treating, ameliorating and/or preventing a condition, disorder or disease associated with a G to A mutation can be administered (e.g., to a subject) via various suitable available technologies.

In some embodiments, provided oligonucleotides, e.g., single-stranded oligonucleotide for site-directed editing of a nucleotide in a target RNA sequences, can be administered as a pharmaceutical composition, e.g., for treating, ameliorating and/or preventing conditions, disorders or diseases. In some embodiments, provided oligonucleotides comprise at least one chirally controlled internucleotidic linkage. In some embodiments, provided oligonucleotide compositions are chirally controlled.

Among other things, technologies, e.g., oligonucleotides and compositions thereof, of the present disclosure can provide various improvements and advantages compared to reference technologies (e.g., absence or low levels of chiral control (e.g., stereorandom oligonucleotide compositions (e.g., of oligonucleotides of the same base sequence, or the same constitution, etc.)), and/or absence or low levels of certain modifications and patterns thereof (e.g., 2′-F, non-negatively charged internucleotidic linkages, etc.), such as improved stability, delivery, editing efficiency, pharmacokinetics, and/or pharmacodynamics. In some embodiments, a reference oligonucleotide composition is a stereorandom oligonucleotide composition of oligonucleotides with the same base sequence. In some embodiments, a reference oligonucleotide composition is a stereorandom oligonucleotide composition of oligonucleotides with the same constitution (as appreciated by those skilled in the art, in some embodiments, various salt forms may be properly considered to be of the same constitution). In some embodiments, a reference oligonucleotide is an oligonucleotide comprising no non-negatively charged internucleotidic linkages. In some embodiments, a reference oligonucleotide comprises no n001. In some embodiments, a reference oligonucleotide composition is a composition of oligonucleotides comprising no non-negatively charged internucleotidic linkages. In some embodiments, a reference oligonucleotide composition is a composition of oligonucleotides comprising no n001. In some embodiments, provided technologies may be utilized at lower unit or total doses, and/or may be administered with fewer doses and/or longer dose intervals (e.g., to achieve comparable or better effects) compared to reference technologies. In some embodiments, provided technologies can provide long durability of editing. In some embodiments, provided technologies once administered can provide activities, e.g., target editing, at or above certain levels (e.g., levels useful and/or sufficient to provide certain biological and/or therapeutic effects) for a period of time, e.g., about or at least about 2, 3, 4, 5, 6, 7, 8, 9, 10, 15, 20, 25, 30, 35, 40, 45, 50, 60 or more days, or 1, 2, 3, 4, 5, 6, 7, 8, 9, or 10 months, after a last dose. In some embodiments, provided technologies provide low toxicity. In some embodiments, provided technologies may be utilized at higher unit or total doses, and/or may be administered with more doses and/or shorter dose intervals (e.g., to achieve better effects) compared to reference technologies. In some embodiments, a total dose may be administered as a single dose. In some embodiments, a total dose may be administered as two or more single doses. In some embodiments, a total dose administered as a single dose may provide higher maximum editing levels compared to when administered as two or more single doses.

In some cases, patients who have been administered an oligonucleotide as a medicament may experience certain side effects or adverse effects, including: thrombocytopenia, renal toxicity, glomerulonephritis, and/or coagulation abnormalities; genotoxicity, repeat-dose toxicity of target organs and pathologic effects; dose response and exposure relationships; chronic toxicity; juvenile toxicity; reproductive and developmental toxicity; cardiovascular safety; injection site reactions; cytokine response complement effects; immunogenicity; and/or carcinogenicity. In some embodiments, an additional therapeutic agent is administered to counter-act a side effect or adverse effect of administration of an oligonucleotide. In some embodiments, a particular single-stranded oligonucleotide for site-directed editing of a nucleotide in a target RNA sequence can have a reduced capability of eliciting a side effect or adverse effect, compared to a different single-stranded oligonucleotide for site-directed editing of a nucleotide in a target RNA sequence.

In some embodiments, an additional therapeutic agent can be administered to the patient in order to control or alleviate one or more side effects or adverse effects associated with administration of an oligonucleotide.

In some embodiments, an oligonucleotide and one or more additional therapeutic agent can be administered to a patient (in any order), wherein the additional therapeutic agent can be administered to the patient in order to control or alleviate one or more side effects or adverse effects associated with administration of the oligonucleotide.

In some embodiments, an oligonucleotide and one or more additional therapeutic agent can be administered to a patient (in any order), wherein the additional therapeutic agent can be administered to the patient in order to control or alleviate one or more side effects or adverse effects associated with administration of the oligonucleotide.

In some embodiments, an oligonucleotide and one or more additional therapeutic agent can be administered to a patient (in any order), wherein the additional therapeutic agent can be administered to the patient in order to control or alleviate one or more side effects or adverse effects associated with administration of the oligonucleotide, and wherein the oligonucleotide operates via any biochemical mechanism, including but not limited to: decreasing the level, expression and/or activity of a target gene or a gene product thereof, increasing or decreasing skipping of one or more exons in a target gene mRNA, an ADAR-mediated deamination, a RNaseH-mediated mechanism, a steric hindrance-mediated mechanism, and/or a RNA interference-mediated mechanism, wherein the oligonucleotide is single- or double-stranded.

In some embodiments, an oligonucleotide composition and one or more additional therapeutic agent can be administered to a patient (in any order), wherein the additional therapeutic agent can be administered to the patient in order to control or alleviate one or more side effects or adverse effects associated with administration of the oligonucleotide composition, and wherein the oligonucleotide composition can be chirally controlled or comprises at least one chirally controlled internucleotidic linkage (including but not limited to a chirally controlled phosphorothioate).

Various conditions, disorders, or diseases can benefit from adenosine editing, including those are associated with a G to A mutation, e.g., Cystic fibrosis, Hurler Syndrome, alpha-1-antitrypsin (A1AT) deficiency, Parkinson's disease, Alzheimer's disease, albinism, Amyotrophic lateral sclerosis, Asthma, β-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and various cancers.

In some embodiments, a condition, disorder or disease is Alpha-1 antitrypsin (AIAT) deficiency (AATD).

Alpha-1 antitrypsin (AIAT) deficiency (AATD) is a genetic disease reportedly caused by defects in the SERPINA1 gene (also known as PI; AIA; AAT; PIl; AIAT; PR02275; and alpha1AT). Severe AIAT deficiency is associated with various phenotypes including lung and liver phenotypes.

AIAT deficiency is reportedly one of the most common genetic diseases in subjects of Northern European descent. Prevalence of severe ‘AAT deficiency in the U.S. alone is 80,000-100,000. Similar numbers are estimated to be found in the EU. The worldwide estimate for severe AAT deficiency has been pegged at 3 million people. A IAT deficiency causes emphysema, with subjects developing emphysema in their third or fourth decade. AIAT deficiency can also cause liver failure and hepatocellular carcinoma, with up to 30% of subjects with severe AIAT deficiency developing significant liver disease, including cirrhosis, fulminant liver failure, and hepatocellular carcinoma.

A mutation (i.e., c. 1024G>A) in SERPINA1 gene leads to a glutamate to lysine substitution at amino acid position 342 (E342K, “Z mutation”) of the mature AIAT protein. This missense mutation affect protein conformation and secretion leading to reduced circulating levels of AIAT. Alleles carrying the Z mutation are identified as PiZ alleles. Subjects homozygous for the PiZ allele are termed PiZZ carriers, and express 10-15% of normal levels of serum AIAT. Approximately 95% of subjects who are symptomatic for AIAT deficiency have the PiZZ genotype. Subjects heterozygous for the Z mutation are termed PiMZ mutants, and express 60% of normal levels of serum A1AT. Of those diagnosed, 90% of patients with severe AAT deficiency have the ZZ mutation. About between 30,000 and 50,000 individuals in the United States have the PiZZ genotype.

The pathophysiology of A1AT deficiency can vary by the organ affected. Liver disease is reported to be due to a gain-of-function mechanism. Abnormally folded A1AT, especially Z-type A1AT (Z-AT), aggregates and polymerizes within hepatocytes. A1AT inclusions are found in PiZZ subjects and are thought to cause cirrhosis and, in some cases, hepatocellular carcinoma. Evidence for the gain-of-function mechanism in liver disease is supported by null homozygotes. These subjects produce no A1AT and do not develop hepatocyte inclusions or liver disease.

It is reported that AIAT deficiency leads to liver disease in up to about 50% of AIAT subjects and leads to severe liver disease in up to about 30% of subjects. Liver disease may manifest as: (a) cirrhosis during childhood that is self-limiting, (b) severe cirrhosis during childhood or adulthood that requires liver transplantation or leads to death and (c) hepatocellular carcinoma that is often deadly. The onset of liver disease is reported to be bi-modal, predominantly affecting children or adults. Childhood disease is self-limiting in many cases but may be led to end-stage, deadly cirrhosis. It is reported that up to about 18% of subjects with the PiZZ genotype may develop clinically significant liver abnormalities during childhood. Approximately 2% of PiZZ subjects are reported to develop severe liver cirrhosis leading to death during childhood (Sveger 1988; Volpert 2000). Adult-onset liver disease may affect subjects with all genotypes, but presents earlier in subjects with the PiZZ genotype. Approximately 2-10% of AIAT deficient subjects are reported to develop adult-onset liver disease.

Lung disease associated with AIAT deficiency is currently treated with intravenous administration of human-derived replacement AIAT protein, but in addition to being costly and requiring frequent injections over a subject's entire lifetime, this approach is only partially effective. AIAT-deficient subjects with hepatocellular carcinoma are currently treated with chemotherapy and surgery, but there is no satisfactory approach for preventing the potentially deadly liver manifestations of AIAT deficiency.

Among other things, the present disclosure recognizes a need for improved treatment of AIAT deficiency, e.g., including liver and lung manifestations thereof. In some embodiments, the present disclosure provides technologies for preventing or treating conditions, disorders or diseases associated Alpha-1 antitrypsin (AIAT) deficiency, e.g., by providing oligonucleotides and/or compositions that can covert the A mutation to I which can be read as G during protein translation and thus correcting the G to A mutation for protein translation. Among other things, alteration of SERPINA1 in one or more of hepatocytes can prevent the progression of liver disease in subjects with AIAT deficiency by reducing or eliminating production of the toxic Z protein (Z-AAT). In certain embodiments, Z protein production is eliminated or reduced by utilizing provided technologies. In certain embodiments, the disease is cured, does not progress, or has delayed progression compared to a subject who has not received the therapy.

In certain embodiments, technologies as described herein can provide a selective advantage to survival of one or more of treated hepatocytes. In certain embodiments, a target cell is modified. In some embodiments, cells treated with technologies herein may not produce toxic Z protein. In some embodiments, diseased cells that are not modified produce toxic Z proteins and may undergo apoptosis secondary to endoplasmic reticulum (ER) stress induced by Z protein misfolding. In certain embodiments, after treatment using the provided technologies, treated cells will survive and untreated cells will die. This selective advantage can drive eventual colonization of hepatocytes with the majority being SERPINA1 corrected cells.

In some embodiments, an oligonucleotide, when administered to a patient suffering from or susceptible to a condition, disorder or disease that is associated with a G to A mutation is capable of reducing at least one symptom of the condition, disorder or disease and/or capable of delaying or preventing the onset, worsening, and/or reducing the rate and/or degree of worsening of at least one symptom of the condition, disorder or disease that's due to a G to A mutation in a gene or gene product.

In some embodiments, provided technologies can provide editing of two or more sites in a system (e.g., a cell, tissue, organ, animal, etc.) (“multiplex editing”). In some embodiments, provided technologies can target and provide editing of two or more sites of the same transcripts. In some embodiments, provided technologies can target and provide editing of two or more different transcripts, either from the same nucleic acid or different nucleic acids. In some embodiments, provided technologies can target and provide editing of transcripts from two or more different nucleic acids. In some embodiments, provided technologies can target and provide editing of transcripts from two or more different genes. In some embodiments, of the targets simultaneously edited, each is independently at a biologically and/or therapeutically relevant level. In some embodiments, in multiplex editing one or more or all targets are independently edited at a comparable level as editing conducted individually under comparable conditions. In some embodiments, multiplex editing are performed utilizing two or more separate compositions, each of which independently target one or more targets. In some embodiments, compositions are administered concurrently. In some embodiments, compositions are administered with suitable intervals. In some embodiments, one or more compositions are administered prior or subsequently to one or more other compositions. In some embodiments, multiplex editing are performed utilizing a single composition, e.g., a composition comprising two or more pluralities of oligonucleotides, wherein the pluralities target different targets. In some embodiments, each plurality independently targets a different adenosine. In some embodiments, each plurality independently targets a different transcript. In some embodiments, each plurality independently targets a different gene. In some embodiments, two or more pluralities may target the same target, but the pluralities together target the desired targets.

As described herein, provided technologies can provide a number of advantages. For example, in some embodiments, provided technologies are safer than technologies that act on DNA, as provided technologies can provide RNA edits that are both reversible and tunable (e.g., through adjusting of doses). Additionally and alternatively, as demonstrated herein, provided technologies can provide high levels of editing in systems expressing endogenous ADAR proteins thus avoiding the requirement of introduction of exogenous proteins in various instances. Still further, provided technologies do not require complex oligonucleotides that depend on ancillary delivery vehicles, such as viral vectors or lipid nanoparticles, as utilized in many other technologies, particularly for application beyond cell culture. In some embodiments, provided technologies can provide sequence-specific A-to-I RNA editing with high efficiency using endogenous ADAR enzymes and can be delivered to various systems, e.g., cells, in the absence of artificial delivery agents.

Those skilled in the art reading the present disclosure will understand that provided oligonucleotides and compositions thereof may be delivered using a number of technologies in accordance with the present disclosure. In some embodiments, provided oligonucleotides and compositions may be delivered via transfection or lipofeciton. In some embodiments, provided oligonucleotides and compositions thereof may be delivered in the absence of delivery aids, such as those utilized in transfection or lipofection. In some embodiments, provided oligonucleotides and compositions may be delivered via transfection or lipofeciton. In some embodiments, provided oligonucleotides and compositions thereof are delivered with gymnotic delivery. In some embodiments, provided oligonucleotides comprise additional chemical moieties that can facilitate delivery. For example, in some embodiments, additional chemical moieties are or comprise ligand moieties (e.g., N-acetylgalactosamine (GalNAc)) for receptors (e.g., asialoglycoprotein receptors). In some embodiments, provided oligonucleotides and compositions thereof can be delivered through GalNAc-mediated delivery.

Among other things, the present disclosure provides the following Embodiments as examples:

1. An oligonucleotide comprising:

a first domain; and

a second domain,

wherein:

the first domain comprises one or more 2'—F modifications;

the second domain comprises one or more sugars that do not have a 2′-F modification.

2. An oligonucleotide comprising one or more modified sugars and/or one or more modified internucleotidic linkages, wherein the oligonucleotide comprises a first domain and a second domain each independently comprising one or more nucleobases.
3. The oligonucleotide of Embodiment 1 or 2, wherein when the oligonucleotide is contacted with a target nucleic acid comprising a target adenosine in a system, a target adenosine in the target nucleic acid is modified.
4. The oligonucleotide of Embodiment 1 or 2, wherein when the oligonucleotide is contacted with a target nucleic acid comprising a target adenosine in a system, level of the target nucleic acid is reduced compared to absence of the product or presence of a reference oligonucleotide.
5. The oligonucleotide of Embodiment 1 or 2, wherein when the oligonucleotide is contacted with a target nucleic acid comprising a target adenosine in a system, splicing of the target nucleic acid or a product thereof is altered compared to absence of the oligonucleotide or presence of a reference oligonucleotide.
6. The oligonucleotide of Embodiment 1 or 2, wherein when the oligonucleotide is contacted with a target nucleic acid comprising a target adenosine in a system, level of a product of the target nucleic acid is altered compared to absence of the product or presence of a reference oligonucleotide.
7. The oligonucleotide of any one of Embodiments 4-6, wherein the target nucleic acid is modified.
8. The oligonucleotide of any one of Embodiments 3-7, wherein level of a product is increased, wherein the product is or is encoded by a nucleic acid which is otherwise identical to the target nucleic acid but the target adenosine is modified.
9. The oligonucleotide of any one of Embodiments 3-7, wherein level of a product is increased, wherein the product is or is encoded by a nucleic acid which is otherwise identical to the target nucleic acid but the target adenosine is replaced with inosine.
10. The oligonucleotide of any one of Embodiments 3-7, wherein level of a product is increased, wherein the product is or is encoded by a nucleic acid which is otherwise identical to the target nucleic acid but the adenine of the target adenosine is replaced with guanine.
11. The oligonucleotide of any one of Embodiments 8-10, wherein the product is a protein.
12. The oligonucleotide of any one of the preceding Embodiments, wherein the target adenosine is a mutation from guanine.
13. The oligonucleotide of any one of the preceding Embodiments, wherein the target adenosine is more associated with a condition, disorder or disease than a guanine at the same position.
14. The oligonucleotide of any one of the preceding Embodiments, wherein the oligonucleotide is capable of forming a double-stranded complex with the target nucleic acid.
15. The oligonucleotide of Embodiment 3-14, wherein a target nucleic acid or a portion thereof is or comprises RNA.
16. The oligonucleotide of any one of Embodiments 3-15, wherein the target adenosine is of an RNA.
17. The oligonucleotide of any one of Embodiments 3-16, wherein the target adenosine is modified, and the modification is or comprises deamination of the target adenosine.
18. The oligonucleotide of any one of Embodiments 3-17, wherein the target adenosine is modified and the modification is or comprises conversion of the target adenosine to an inosine.
19. The oligonucleotide of any one of Embodiments 3-18, wherein the modification is promoted by an ADAR protein.
20. The oligonucleotide of any one of Embodiments 3-19, wherein the system is an in vitro or ex vivo system comprising an ADAR protein.
21. The oligonucleotide of any one of Embodiments 3-19, wherein the system is or comprises a cell that comprises or expresses an ADAR protein.
22. The oligonucleotide of any one of Embodiments 3-19, wherein the system is a subject comprising a cell that comprises or expresses an ADAR protein.
23. The oligonucleotide of any one of Embodiments 19-22, wherein the ADAR protein is ADAR1.
24. The oligonucleotide of any one of Embodiments 19-22, wherein the ADAR protein is ADAR2.
25. The oligonucleotide of any one of the preceding Embodiments, wherein the oligonucleotide has a length of about 10-200 (e.g., about 10-20, 10-30, 10-40, 10-50, 10-60, 10-70, 10-80, 10-90, 10-100, 10-120, 10-150, 20-30, 20-40, 20-50, 20-60, 20-70, 20-80, 20-90, 20-100, 20-120, 20-150, 20-200, 25-30, 25-40, 25-50, 25-60, 25-70, 25-80, 25-90, 25-100, 25-120, 25-150, 25-200, 30-40, 30-50, 30-60, 30-70, 30-80, 30-90, 30-100, 30-120, 30-150, 30-200, 10, 20, 25, 26, 27, 28, 29, 30, 31, 32, 33, 34, 35, 36, 37, 38, 39, 40, 45, 50, 60, etc.) nucleobases.
26. The oligonucleotide of any one of the preceding Embodiments, wherein the oligonucleotide has a length of about 26-35 nucleobases.
27. The oligonucleotide of any one of the preceding Embodiments, wherein the oligonucleotide has a length of about 29-35 nucleobases.
28. The oligonucleotide of any one of the preceding Embodiments, wherein the base sequence of the oligonucleotide is complementary to a base sequence of a portion of the target nucleic acid comprising the target adenosine with 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) mismatches which are not Watson-Crick base pairs.
29. The oligonucleotide of Embodiment 28, wherein one or more mismatches are independently a wobble base paring.
30. The oligonucleotide of any one of Embodiments 28-29, wherein the complementarity is about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.).
31. The oligonucleotide of any one of Embodiments 28-29, wherein the complementarity is about 90%-100% or about 95-100%.
32. The oligonucleotide of any one of Embodiments 28-29, wherein the complementarity is 100%.
33. The oligonucleotide of any one of Embodiments 28-29, wherein the complementarity is 100% except at a nucleoside opposite to a target nucleoside (e.g., adenosine).
34. The oligonucleotide of any one of the preceding Embodiments, wherein the oligonucleotide consists of a first domain and a second domain.
35. The oligonucleotide of any one of the preceding Embodiments, wherein the first domain has a length of about 2-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases.
36. The oligonucleotide of any one of the preceding Embodiments, wherein the first domain has a length of about 10-25 nucleobases.
37. The oligonucleotide of any one of the preceding Embodiments, wherein the first domain has a length of about 15 nucleobases.
38. The oligonucleotide of any one of the preceding Embodiments, wherein the first domain comprises one or more (e.g., 1-10, 1, 2, 3, 4, 5, 6, 7, 8, 9, or 10, etc.) mismatches when the oligonucleotide is aligned with a target nucleic acid for complementarity.
39. The oligonucleotide of any one of the preceding Embodiments, wherein the first domain comprises two or more mismatches when the oligonucleotide is aligned with a target nucleic acid for complementarity.
40. The oligonucleotide of any one of Embodiments 1-35, wherein the first domain comprises one and no more than one mismatch when the oligonucleotide is aligned with a target nucleic acid for complementarity.
41. The oligonucleotide of any one of Embodiments 1-35, wherein the first domain comprises two and no more than two mismatches when the oligonucleotide is aligned with a target nucleic acid for complementarity.
42. The oligonucleotide of any one of the preceding Embodiments, wherein the first domain comprises one or more (e.g., 1-10, 1, 2, 3, 4, 5, 6, 7, 8, 9, or 10, etc.) bulges when the oligonucleotide is aligned with a target nucleic acid for complementarity.
43. The oligonucleotide of Embodiment 42, wherein each bulge independently comprises one or more base pairs that are not Watson-Crick or wobble pairs.
44. The oligonucleotide of any one of the preceding Embodiments, wherein the first domain comprises one or more (e.g., 1-10, 1, 2, 3, 4, 5, 6, 7, 8, 9, or 10, etc.) wobble pairs when the oligonucleotide is aligned with a target nucleic acid for complementarity.
45. The oligonucleotide of any one of the preceding Embodiments, wherein the first domain comprises two or more wobble pairs when the oligonucleotide is aligned with a target nucleic acid for complementarity.
46. The oligonucleotide of any one of the preceding Embodiments, wherein the first domain comprises two and no more than two wobble pairs when the oligonucleotide is aligned with a target nucleic acid for complementarity.
47. The oligonucleotide of any one of Embodiments 1-35, wherein the first domain is fully complementary to a target nucleic acid.
48. The oligonucleotide of any one of the preceding Embodiments, wherein the first domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) sugars with 2′-F modification.
49. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in the first domain independently comprise a 2′-F modification.
50. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the first domain independently comprise a 2′-F modification.
51. The oligonucleotide of any one of the preceding Embodiments, wherein no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in the first domain comprises 2′-OMe.
52. The oligonucleotide of any one of the preceding Embodiments, wherein no more than about 50% of sugars in the first domain comprises 2′-OMe.
53. The oligonucleotide of any one of the preceding Embodiments, wherein no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in the first domain comprises 2′-OR, wherein R is optionally substituted C₁₋₆ aliphatic.
54. The oligonucleotide of any one of the preceding Embodiments, wherein no more than about 50% of sugars in the first domain comprises 2′-OR, wherein R is optionally substituted C₁₋₆ aliphatic.
55. The oligonucleotide of any one of the preceding Embodiments, wherein no more than about 1%-95% (e.g., no more than about 1%, 5%, 10%, 15%, 20%, 25%, 30%, 35%, 40%, 45%, 50%, 55%, 60%, 65%, 70%, 75%, 80%, 85%, 90%, 95%, etc.) of sugars in the first domain comprises 2′-OR.
56. The oligonucleotide of any one of the preceding Embodiments, wherein no more than about 50% of sugars in the first domain comprises 2′-OR.
57. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OR modification, wherein R is optionally substituted C₁₋₆ aliphatic.
58. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-MOE modification.
59. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OMe modification.
60. The oligonucleotide of any one of the preceding Embodiments, wherein the first about 1-5, e.g., 1, 2, 3, 4, or 5 sugars from the 5′-end of a first domain is independently a 2′-OR modified sugar, wherein R is independently optionally substituted C₁₋₆ aliphatic.
61. The oligonucleotide of any one of the preceding Embodiments, wherein the first about 1-5, e.g., 1, 2, 3, 4, or 5 sugars from the 5′-end of a first domain is independently a 2′-MOE modified sugar.
62. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification, wherein each R is optionally substituted C₁₋₆ aliphatic.
63. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification.
64. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) LNA sugars.
65. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).
66. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-F modification.
67. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) sugars comprising 2′-OH.
68. The oligonucleotide of any one of the preceding Embodiments, wherein the first domain comprises one or more (e.g., about 1-20, 1, 2, 3, 4, 5, 6, 7, 8, 9, 10, 11, 12, 13, 14, 15, 16, 17, 18, 19, 20, etc.) sugars comprising two 2′-H.
69. The oligonucleotide of any one of Embodiments 1-56, wherein no sugar in the first domain comprises 2′-OR.
70. The oligonucleotide of any one of Embodiments 1-56, wherein no sugar in the first domain comprises 2′-OMe.
71. The oligonucleotide of any one of Embodiments 1-56, wherein no sugar in the first domain comprises 2′-OR, wherein R is optionally substituted C₁₋₆ aliphatic.
72. The oligonucleotide of any one of Embodiments 1-56, wherein each sugar in the first domain comprises 2′-F.
73. The oligonucleotide of any one of the preceding Embodiments, wherein the first domain comprise about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages.
74. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in the first domain are modified internucleotidic linkages.
75. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of internucleotidic linkages in the first domain are modified internucleotidic linkages.
76. The oligonucleotide of any one of the preceding Embodiments, wherein each modified internucleotidic linkages is independently a chiral internucleotidic linkage.
77. The oligonucleotide of any one of the preceding Embodiments, wherein each modified internucleotidic linkages is independently a phosphorothioate internucleotidic linkage or a non-negatively charged internucleotidic linkage.
78. The oligonucleotide of any one of the preceding Embodiments, wherein each modified internucleotidic linkages is independently a phosphorothioate internucleotidic linkage or a neutral internucleotidic linkage.
79. The oligonucleotide of any one of the preceding Embodiments, wherein the first domain comprises one or more phosphorothioate internucleotidic linkages.
80. The oligonucleotide of any one of the preceding Embodiments, wherein the first domain comprises 1, 2, 3, 4, or 5 non-negatively charged internucleotidic linkages.
81. The oligonucleotide of any one of the preceding Embodiments, wherein the internucleotidic linkage between the first and the second nucleosides of the first domain is a non-negatively charged internucleotidic linkage.
82. The oligonucleotide of any one of the preceding Embodiments, wherein the internucleotidic linkage between the last and the second last nucleosides of the first domain is a non-negatively charged internucleotidic linkage.
83. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the first domain is chirally controlled.
84. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the first domain is chirally controlled.
85. The oligonucleotide of any one of the preceding Embodiments, wherein the internucleotidic linkage between the first and the second nucleosides of the first domain is chirally controlled.
86. The oligonucleotide of any one of the preceding Embodiments, wherein the internucleotidic linkage between the last and the second last nucleosides of the first domain is chirally controlled.
87. The oligonucleotide of any one of the preceding Embodiments, wherein each chiral internucleotidic linkage is independently a chirally controlled internucleotidic linkage.
88. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the first domain is Sp.
89. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the first domain is Sp.
90. The oligonucleotide of any one of the preceding Embodiments, wherein each chiral internucleotidic linkages in the first domain is Sp.
91. The oligonucleotide of any one of Embodiments 1-89, wherein the internucleotidic linkage between the first and the second nucleosides of the first domain is Rp.
92. The oligonucleotide of any one of Embodiments 1-89 and 91, wherein the internucleotidic linkage between the last and the second last nucleosides of the first domain is Rp.
93. The oligonucleotide of any one of the preceding Embodiments, wherein each internucleotidic linkage in the first domain is independently a modified internucleotidic linkage.
94. The oligonucleotide of any one of Embodiments 1-92, wherein the first domain comprises one or more natural phosphate linkages.
95. The oligonucleotide of any one of the preceding Embodiments, wherein the first domain can recruit, or promotes or contributes to recruitment of, an ADAR protein to a target nucleic acid.
96. The oligonucleotide of any one of the preceding Embodiments, wherein the first domain can interact, or promotes or contributes to interaction of, an ADAR protein with a target nucleic acid.
97. The oligonucleotide of any one of the preceding Embodiments, wherein the first domain contacts with a RNA binding domain (RBD) of ADAR.
98. The oligonucleotide of any one of the preceding Embodiments, wherein the first domain does not substantially contact with a second RBD domain of ADAR.
99. The oligonucleotide of any one of the preceding Embodiments, wherein the first domain does not substantially contact with a catalytic domain which has a deaminase activity, of ADAR.
100. The oligonucleotide of any one of the preceding Embodiments, wherein the second domain has a length of about 2-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases.
101. The oligonucleotide of any one of the preceding Embodiments, wherein the second domain has a length of about 1-7 nucleobases.
102. The oligonucleotide of any one of the preceding Embodiments, wherein the second domain has a length of about 5-15 nucleobases.
103. The oligonucleotide of any one of the preceding Embodiments, wherein the second domain has a length of about 10-25 nucleobases.
104. The oligonucleotide of any one of the preceding Embodiments, wherein the second domain has a length of about 15 nucleobases.
105. The oligonucleotide of any one of the preceding Embodiments, wherein the second domain comprises one or more (e.g., 1-10, 1, 2, 3, 4, 5, 6, 7, 8, 9, or 10, etc.) mismatches when the oligonucleotide is aligned with a target nucleic acid for complementarity.
106. The oligonucleotide of any one of the preceding Embodiments, wherein the second domain comprises two or more mismatches when the oligonucleotide is aligned with a target nucleic acid for complementarity.
107. The oligonucleotide of any one of Embodiments 1-100, wherein the second domain comprises one and no more than one mismatch when the oligonucleotide is aligned with a target nucleic acid for complementarity.
108. The oligonucleotide of any one of Embodiments 1-100, wherein the second domain comprises two and no more than two mismatches when the oligonucleotide is aligned with a target nucleic acid for complementarity.
109. The oligonucleotide of any one of the preceding Embodiments, wherein the second domain comprises one or more (e.g., 1-10, 1, 2, 3, 4, 5, 6, 7, 8, 9, or 10, etc.) bulges when the oligonucleotide is aligned with a target nucleic acid for complementarity.
110. The oligonucleotide of Embodiment 109, wherein each bulge independently comprises one or more base pairs that are not Watson-Crick or wobble pairs.
111. The oligonucleotide of any one of the preceding Embodiments, wherein the second domain comprises one or more (e.g., 1-10, 1, 2, 3, 4, 5, 6, 7, 8, 9, or 10, etc.) wobble pairs when the oligonucleotide is aligned with a target nucleic acid for complementarity.
112. The oligonucleotide of any one of the preceding Embodiments, wherein the second domain comprises two or more wobble pairs when the oligonucleotide is aligned with a target nucleic acid for complementarity.
113. The oligonucleotide of any one of the preceding Embodiments, wherein the second domain comprises two and no more than two wobble pairs when the oligonucleotide is aligned with a target nucleic acid for complementarity.
114. The oligonucleotide of any one of Embodiments 1-100, wherein the second domain is fully complementary to a target nucleic acid.
115. The oligonucleotide of any one of the preceding Embodiments, wherein the second domain comprise a nucleoside opposite to a target adenosine when the oligonucleotide is aligned with a target nucleic acid for complementarity.
116. The oligonucleotide of Embodiment 115, wherein the opposite nucleobase is optionally substituted or protected U, or is an optionally substituted or protected tautomer of U.
117. The oligonucleotide of Embodiment 115, wherein the opposite nucleobase is U.
118. The oligonucleotide of Embodiment 115, wherein the opposite nucleobase is optionally substituted or protected C, or is an optionally substituted or protected tautomer of C.
119. The oligonucleotide of Embodiment 115, wherein the opposite nucleobase is C.
120. The oligonucleotide of Embodiment 115, wherein the opposite nucleobase is optionally substituted or protected A, or is an optionally substituted or protected tautomer of A.
121. The oligonucleotide of Embodiment 115, wherein the opposite nucleobase is A.
122. The oligonucleotide of Embodiment 115, wherein the opposite nucleobase is optionally substituted or protected nucleobase of pseudoisocytosine, or is an optionally substituted or protected tautomer of the nucleobase of pseudoisocytosine.
123. The oligonucleotide of Embodiment 115, wherein the opposite nucleobase is the nucleobase of pseudoisocytosine.
124. The oligonucleotide of any one of the preceding Embodiments, wherein the oligonucleotide comprises a nucleobase BA, wherein BA is or comprises Ring BA or a tautomer thereof, wherein Ring BA is an optionally substituted, 5-20 membered, monocyclic, bicyclic or polycyclic ring having 0-10 hetereoatoms.
125. An oligonucleotide, wherein the oligonucleotide comprises a nucleobase BA, wherein BA is or comprises Ring BA or a tautomer thereof, wherein Ring BA is an optionally substituted, 5-20 membered, monocyclic, bicyclic or polycyclic ring having 0-10 hetereoatoms.
126. The oligonucleotide of Embodiment 115, wherein the nucleobase is BA, wherein BA is or comprises Ring BA or a tautomer thereof, wherein Ring BA is an optionally substituted, 5-20 membered, monocyclic, bicyclic or polycyclic ring having 0-10 hetereoatoms.
127. The oligonucleotide of any one of Embodiments 124-126, wherein BA has weaker hydrogen bonding with the target adenine of the adenosine compared to U.
128. The oligonucleotide of any one of Embodiments 124-127, wherein BA forms fewer hydrogen bonds with the target adenine of the adenosine compared to U.
129. The oligonucleotide of any one of Embodiments 124-128, wherein BA forms one or more hydrogen bonds with one or more amino acid residues of ADAR which residues form one or more hydrogen bonds with U opposite to a target adenosine.
130. The oligonucleotide of any one of Embodiments 124-129, wherein BA forms one or more hydrogen bonds with each amino acid residue of ADAR that forms one or more hydrogen bonds with U opposite to a target adenosine.
131. The oligonucleotide of any one of Embodiments 124-130, wherein Ring BA comprises [symbol] X² [symbol] X³ [symbol] .
132. The oligonucleotide of any one of Embodiments 124-130, wherein Ring BA comprises [symbol] X² [symbol] X³ [symbol] X⁴ [symbol] .
133. The oligonucleotide of any one of Embodiments 124-130, wherein Ring BA comprises —X¹( [symbol] ) [symbol] X² [symbol] X³ [symbol] .
134. The oligonucleotide of any one of Embodiments 124-130, wherein Ring BA comprises —X¹( [symbol] ) [symbol] X² [symbol] X³ [symbol] X⁴.
135. The oligonucleotide of any one of Embodiments 124-134, wherein Ring BA has the structure of formula BA-I.
136. The oligonucleotide of any one of Embodiments 124-134, wherein Ring BA has the structure of formula BA-I-a.
137. The oligonucleotide of any one of Embodiments 124-134, wherein Ring BA has the structure of formula BA-I-b.
138. The oligonucleotide of any one of Embodiments 124-134, wherein Ring BA has the structure of formula BA-II.
139. The oligonucleotide of any one of Embodiments 124-134, wherein Ring BA has the structure of formula BA-II-a.
140. The oligonucleotide of any one of Embodiments 124-134, wherein Ring BA has the structure of formula BA-II-b.
141. The oligonucleotide of any one of Embodiments 124-134, wherein Ring BA has the structure of formula BA-III.
142. The oligonucleotide of any one of Embodiments 124-134, wherein Ring BA has the structure of formula BA-III-a.
143. The oligonucleotide of any one of Embodiments 124-134, wherein Ring BA has the structure of formula BA-III-b.
144. The oligonucleotide of any one of Embodiments 124-143, wherein each of X¹, X², X³, X⁴, X⁵, X⁶, X^{1′}, X^{2′}, X^{3′}, X^{4′}, X^{5′}, X^{6′}, and X^{7′} is independently and optionally substituted when it is —CH═, —C(OH)═, —C(—NH₂)═, —CH₂—, —C(═NH)—, or —NH—.
145. The oligonucleotide of any one of Embodiments 131-144, wherein X¹ is —N(−)—.
146. The oligonucleotide of any one of Embodiments 131-144, wherein X¹ is —C(−)=.
147. The oligonucleotide of any one of Embodiments 131-146, wherein X² is —C(O)—.
148. The oligonucleotide of any one of Embodiments 131-147, wherein X³ is —NR′—.
149. The oligonucleotide of any one of Embodiments 131-148, wherein X³ is optionally substituted —NH—.
150. The oligonucleotide of any one of Embodiments 131-148, wherein X³ is —NH—.
151. The oligonucleotide of any one of Embodiments 131-150, wherein X⁴ is —C(R^{B4})═, —C(—N(R^{B4})₂)═, C(R^{B4})₂—, or —C(═NR^{B4})—.
152. The oligonucleotide of any one of Embodiments 131-150, wherein X⁴ is —C(R^{B4})═.
153. The oligonucleotide of any one of Embodiments 131-150, wherein X⁴ is optionally substituted —CH═.
154. The oligonucleotide of any one of Embodiments 131-150, wherein X⁴ is —CH═.
155. The oligonucleotide of any one of Embodiments 131-150, wherein X⁴ is —C(⁻N(R^{B4})₂)═.
156. The oligonucleotide of any one of Embodiments 131-150, wherein X⁴ is optionally substituted —C(—NH₂)═.
157. The oligonucleotide of any one of Embodiments 131-150, wherein X⁴ is —C(—NH₂)═.
158. The oligonucleotide of any one of Embodiments 131-150, wherein X⁴ is —C(—N═CHNR₂)═.
159. The oligonucleotide of any one of Embodiments 131-150, wherein X⁴ is —C(—N═CHN(CH₃)₂)═.
160. The oligonucleotide of any one of Embodiments 131-150, wherein X⁴ is —C(—NHR′)═.
161. The oligonucleotide of any one of Embodiments 131-150, wherein X⁴ is —C(R^{B4})₂—.
162. The oligonucleotide of any one of Embodiments 131-150, wherein X⁴ is optionally substituted —CH₂—.
163. The oligonucleotide of any one of Embodiments 131-150, wherein X⁴ is —CH₂—.
164. The oligonucleotide of any one of Embodiments 131-150, wherein X⁴ is optionally substituted —C(═NH)—.
165. The oligonucleotide of any one of Embodiments 131-150, wherein X⁴ is —C(═NR^{B4})—.
166. The oligonucleotide of any one of Embodiments 131-150, wherein X⁴ is —C(O)═, wherein the oxygen atom has a weaker hydrogen bond acceptor than the corresponding —C(O)— in U.
167. The oligonucleotide of any one of Embodiments 131-150, wherein X⁴ is —C(O)═, wherein the oxygen atom forms an intromolecular hydrogen bond.
168. The oligonucleotide of any one of Embodiments 131-150, wherein X⁴ is —C(O)═, wherein the oxygen atom forms an hydrogen bond with a hydrogen within the same nucleobase.
169. The oligonucleotide of any one of Embodiments 138-168, wherein X⁵ is —C(R^{B5})₂—.
170. The oligonucleotide of any one of Embodiments 138-168, wherein X⁵ is optionally substituted —CH₂—.
171. The oligonucleotide of any one of Embodiments 138-168, wherein X⁵ is —CH₂—.
172. The oligonucleotide of any one of Embodiments 138-168, wherein X⁵ is —C(R^{B5})═.
173. The oligonucleotide of any one of Embodiments 138-168, wherein X⁵ is optionally substituted —C(—NO₂)═.
174. The oligonucleotide of any one of Embodiments 138-168, wherein X⁵ is optionally substituted —CH═.
175. The oligonucleotide of any one of Embodiments 138-168, wherein X⁵ is —CH═.
176. The oligonucleotide of any one of Embodiments 138-168, wherein X⁵ is —C(-L^{B5}-R^{B51})═, wherein R^{B51} is —R′, —N(R′)₂, —OR′, or —SR′.
177. The oligonucleotide of any one of Embodiments 138-168, wherein X⁵ is —C(-L^{B5}-R^{B51})═, wherein R^{B51} is —N(R′)₂, —OR′, or —SR′.
178. The oligonucleotide of any one of Embodiments 138-168, wherein X⁵ is —C(-L^{B5}-R^{B51})═, wherein R^{B51} is —NHR′.
179. The oligonucleotide of any one of Embodiments 176-178, wherein L^{B5} is or comprises —C(O).
180. The oligonucleotide of any one of Embodiments 138-168, wherein X⁵ is —N═.
181. The oligonucleotide of any one of Embodiments 178-179, wherein X⁴ is —C(O)═, wherein the oxygen atom forms a hydrogen bond with a hydrogen of —NHR′, —OH or —SH in R^{B51}.
182. The oligonucleotide of any one of Embodiments 124-134, wherein Ring BA has the structure of formula BA-IV.
183. The oligonucleotide of any one of Embodiments 124-134, wherein Ring BA has the structure of formula BA-IV-a.
184. The oligonucleotide of any one of Embodiments 124-134, wherein Ring BA has the structure of formula BA-IV-b.
185. The oligonucleotide of any one of Embodiments 124-134, wherein Ring BA has the structure of formula BA-V.
186. The oligonucleotide of any one of Embodiments 124-134, wherein Ring BA has the structure of formula BA-V-a.
187. The oligonucleotide of any one of Embodiments 124-134, wherein Ring BA has the structure of formula BA-V-b.
188. The oligonucleotide of any one of Embodiments 124-134, wherein Ring BA has the structure of formula BA-VI.
189. The oligonucleotide of any one of Embodiments 182-188, wherein each of X¹, X², X³, X⁴, X⁵, X⁶, X^{1′}, X^{2′}, X^{3′}, X^{4′}, X^{5′}, X^{6′}, and X^{7′} is independently and optionally substituted when it is —CH═, —C(OH)═, —C(—NH₂)═, —CH₂—, —C(═NH)—, or —NH—.
190. The oligonucleotide of any one of Embodiments 182-189, wherein X¹ is —N(−)—.
191. The oligonucleotide of any one of Embodiments 182-189, wherein X¹ is —C(−)=.
192. The oligonucleotide of any one of Embodiments 182-191, wherein X² is optionally substituted —CH═.
193. The oligonucleotide of any one of Embodiments 182-191, wherein X² is —CH═.
194. The oligonucleotide of any one of Embodiments 182-191, wherein X² is —C(O)—.
195. The oligonucleotide of any one of Embodiments 182-194, wherein X³ is
196. The oligonucleotide of any one of Embodiments 182-194, wherein X³ is optionally substituted —NH—.
197. The oligonucleotide of any one of Embodiments 182-194, wherein X³ is —NH—.
198. The oligonucleotide of any one of Embodiments 182-197, wherein Ring BA^{A} is 5-membered.
199. The oligonucleotide of any one of Embodiments 182-197, wherein Ring BA^{A} is 6-membered.
200. The oligonucleotide of any one of Embodiments 182-199, wherein Ring BA^{A} is an optionally substituted ring having 1-3 heteroatoms.
201. The oligonucleotide of Embodiment 200, wherein a heteroatom is a nitrogen.
202. The oligonucleotide of any one of Embodiments 200-201, wherein Ring BA^{A} contains two nitrogen.
203. The oligonucleotide of any one of Embodiments 200-201, wherein a heteroatom is oxygen.
204. The oligonucleotide of any one of Embodiments 141-203, wherein X⁶ is —C(R^{B6})═, —C(OR^{B6})═, —C(R^{B6})₂—, or —C(O)—.
205. The oligonucleotide of any one of Embodiments 141-203, wherein X⁶ is —C(R)═, —C(R)₂—, or —C(O)—.
206. The oligonucleotide of any one of Embodiments 141-203, wherein X⁶ is optionally substituted —CH═.
207. The oligonucleotide of any one of Embodiments 141-203, wherein X⁶ is —CH═.
208. The oligonucleotide of any one of Embodiments 141-203, wherein X⁶ is optionally substituted —CH₂—.
209. The oligonucleotide of any one of Embodiments 141-203, wherein X⁶ is —CH₂—.
210. The oligonucleotide of any one of Embodiments 141-203, wherein X⁶ is —C(O)—.
211. The oligonucleotide of any one of Embodiments 115-130, wherein Ring BA comprises [symbol] X^{4′} [symbol] X^{5′} [symbol] .
212. The oligonucleotide of any one of Embodiments 124-130 or 211, wherein Ring BA has the structure of formula BA-VI.
213. The oligonucleotide of Embodiment 211, wherein X^{1′} is —N(−)—.
214. The oligonucleotide of Embodiment 211, wherein X^{1′} is —C(−)=.
215. The oligonucleotide of any one of Embodiments 211-214, wherein X^{2′} is —C(O)—.
216. The oligonucleotide of any one of Embodiments 211-214, wherein X^{2′} is optionally substituted —CH═.
217. The oligonucleotide of any one of Embodiments 211-214, wherein X^{2′} is —CH═.
218. The oligonucleotide of any one of Embodiments 211-214, wherein X^{2′} is —C(−)=.
219. The oligonucleotide of any one of Embodiments 211-217, wherein X^{3′} is —NR′—.
220. The oligonucleotide of any one of Embodiments 211-217, wherein X^{3′} is optionally substituted —NH—.
221. The oligonucleotide of any one of Embodiments 211-217, wherein X^{3′} is —NH—.
222. The oligonucleotide of any one of Embodiments 211-217, wherein X^{3′} is —N═.
223. The oligonucleotide of any one of Embodiments 211-222, wherein X^{4′} is —C(O)═.
224. The oligonucleotide of any one of Embodiments 211-222, wherein X^{4′} is —C(OR^{B4′})═.
225. The oligonucleotide of any one of Embodiments 211-222, wherein X^{4′} is —C(R^{B4′})═.
226. The oligonucleotide of any one of Embodiments 211-222, wherein X^{4′} is optionally substituted —CH═.
227. The oligonucleotide of any one of Embodiments 211-222, wherein X^{4′} is —CH═.
228. The oligonucleotide of any one of Embodiments 211-222, wherein X^{4′} is —C(⁻N(R^{B4′})₂)═.
229. The oligonucleotide of any one of Embodiments 211-222, wherein X^{4′} is optionally substituted —C(—NH₂)═.
230. The oligonucleotide of any one of Embodiments 211-222, wherein X^{4′} is —C(—NH₂)═.
231. The oligonucleotide of any one of Embodiments 211-222, wherein X^{4′} is C(N═CHN(CH₃)₂)═.
232. The oligonucleotide of any one of Embodiments 211-222, wherein X^{4′} is —C(—NC(O)R′)═.
233. The oligonucleotide of any one of Embodiments 211-232, wherein X^{5′} is optionally substituted —NH—.
234. The oligonucleotide of any one of Embodiments 211-232, wherein X^{5′} is —NH—.
235. The oligonucleotide of any one of Embodiments 211-232, wherein X^{5′} is —N═.
236. The oligonucleotide of any one of Embodiments 211-232, wherein X^{5′} is —C(R^{B5′})═.
237. The oligonucleotide of any one of Embodiments 211-232, wherein X^{5′} is optionally substituted —CH═.
238. The oligonucleotide of any one of Embodiments 211-232, wherein X^{5′} is —CH═.
239. The oligonucleotide of any one of Embodiments 211-238, wherein X^{6′} is —C(R^{B6′})═.
240. The oligonucleotide of any one of Embodiments 211-238, wherein X^{6′} is optionally substituted —CH═.
241. The oligonucleotide of any one of Embodiments 211-238, wherein X^{6′} is —CH═.
242. The oligonucleotide of any one of Embodiments 211-238, wherein X^{6′} is —C(O)═.
243. The oligonucleotide of any one of Embodiments 211-238, wherein X^{6′} is —C(OR^{B6′})═.
244. The oligonucleotide of any one of Embodiments 211-238, wherein X^{6′} is —C(OR′)═.
245. The oligonucleotide of any one of Embodiments 211-244, wherein X^{7′} is —C(R^{B7′})═.
246. The oligonucleotide of any one of Embodiments 211-244, wherein X^{7′} is optionally substituted —CH═.
247. The oligonucleotide of any one of Embodiments 211-244, wherein X^{7′} is —CH═.
248. The oligonucleotide of any one of Embodiments 211-244, wherein X^{7′} is optionally substituted —NH—.
249. The oligonucleotide of any one of Embodiments 211-244, wherein X^{7′} is —NH—.
250. The oligonucleotide of any one of Embodiments 211-244, wherein X^{7′} is —N═.
251. The oligonucleotide of any one of Embodiments 124-130, wherein Ring BA is

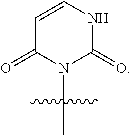 [SMILES: *n1c(=O)cc[nH]c1=O]

252. The oligonucleotide of any one of Embodiments 124-130, wherein Ring BA is

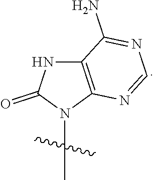 [SMILES: *n1c(=O)[nH]c2c(N)ncnc21]

253. The oligonucleotide of any one of Embodiments 124-130, wherein Ring BA is

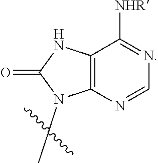

254. The oligonucleotide of any one of Embodiments 124-130, wherein Ring BA is

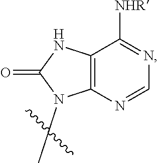

wherein R′ is —C(O)R.
255. The oligonucleotide of any one of Embodiments 124-130, wherein Ring BA is

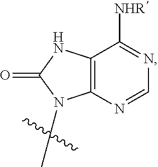

wherein R′ is —C(O)Ph.
256. The oligonucleotide of any one of Embodiments 124-130, wherein Ring BA is

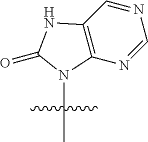 [SMILES: *n1c(=O)[nH]c2cncnc21]

257. The oligonucleotide of any one of Embodiments 124-130, wherein Ring BA is

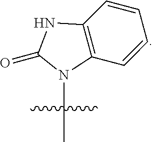 [SMILES: *n1c(=O)[nH]c2ccccc21]

258. The oligonucleotide of any one of Embodiments 124-130, wherein Ring BA is

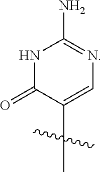 [SMILES: *c1cnc(N)[nH]c1=O]

259. The oligonucleotide of any one of Embodiments 124-130, wherein Ring BA is

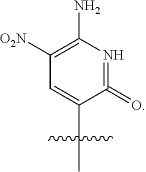

260. The oligonucleotide of any one of Embodiments 124-130, wherein Ring BA is

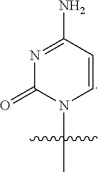 [SMILES: *n1ccc(N)nc1=O]

261. The oligonucleotide of any one of Embodiments 124-130, wherein Ring BA is

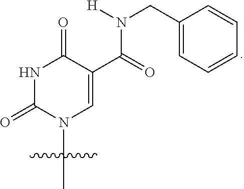 [SMILES: *n1cc(C(=O)NCc2ccccc2)c(=O)[nH]c1=O]

262. The oligonucleotide of any one of Embodiments 124-130, wherein Ring BA is

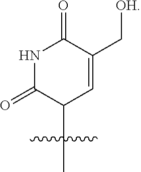 [SMILES: *C1C=C(CO)C(=O)NC1=O]

263. The oligonucleotide of any one of Embodiments 124-130, wherein Ring BA is

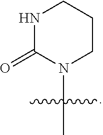 [SMILES: *N1CCCNC1=O]

264. The oligonucleotide of any one of Embodiments 124-130, wherein Ring BA is

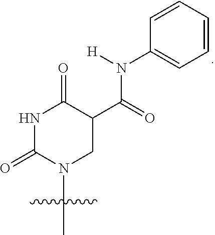 [SMILES: *N1CC(C(=O)Nc2ccccc2)C(=O)NC1=O]

265. The oligonucleotide of any one of Embodiments 124-130, wherein Ring BA is

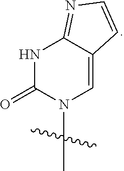 [SMILES: *n1cc2ccnc-2[nH]c1=O]

266. The oligonucleotide of any one of Embodiments 124-130, wherein Ring BA is

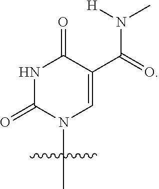 [SMILES: *n1cc(C(=O)NC)c(=O)[nH]c1=O]

267. The oligonucleotide of any one of Embodiments 124-130, wherein Ring BA is

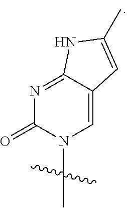 [SMILES: *n1cc2cc(C)[nH]c2nc1=O]

268. The oligonucleotide of any one of Embodiments 124-130, wherein Ring BA is

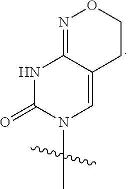 [SMILES: *N1C=C2CCON=C2NC1=O]

269. The oligonucleotide of any one of Embodiments 124-130, wherein Ring BA is

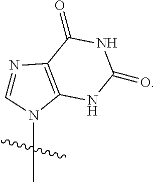 [SMILES: *n1cnc2c(=O)[nH]c(=O)[nH]c21]

270. The oligonucleotide of any one of Embodiments 124-130, wherein Ring BA is

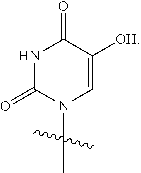 [SMILES: *n1cc(O)c(=O)[nH]c1=O]

271. The oligonucleotide of any one of Embodiments 124-130, wherein Ring BA is

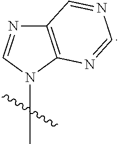 [SMILES: *n1cnc2cncnc21]

272. The oligonucleotide of any one of Embodiments 124-130, wherein Ring BA is

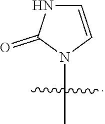 [SMILES: *n1cc[nH]c1=O]

273. The oligonucleotide of any one of Embodiments 124-130, wherein Ring BA is

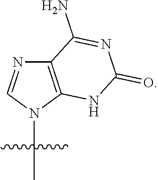 [SMILES: *n1cnc2c(N)nc(=O)[nH]c21]

274. The oligonucleotide of any one of Embodiments 124-130, wherein Ring BA is

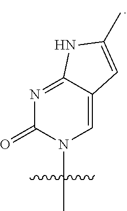 [SMILES: *n1cc2cc(C)[nH]c2nc1=O]

275. The oligonucleotide of any one of Embodiments 124-274, wherein a nucleobase is Ring BA or a tautomer thereof.
276. The oligonucleotide of any one of Embodiments 124-274, wherein a nucleobase is substituted Ring BA or a tautomer thereof.
277. The oligonucleotide of any one of Embodiments 124-274, wherein a nucleobase is optionally substituted Ring BA or a tautomer thereof, wherein each ring —CH═, —CH₂— and —NH— is optionally and independently substituted.
278. The oligonucleotide of any one of Embodiments 124-274, wherein a nucleobase is optionally substituted Ring BA or a tautomer thereof, wherein each ring —CH═ and —CH₂— is optionally and independently substituted.
279. The oligonucleotide of any one of Embodiments 124-274, wherein a nucleobase is optionally substituted Ring BA or a tautomer thereof, wherein each ring —CH═ is optionally and independently substituted.
280. The oligonucleotide of any one of the preceding Embodiments, wherein the second domain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F.
281. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in the second domain are independently modified sugars with a modification that is not 2′-F.
282. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the second domain are independently modified sugars with a modification that is not 2′-F.
283. The oligonucleotide of any one of Embodiments 120-282, wherein the modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
284. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-F modification.
285. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OR modification, wherein R is optionally substituted C₁₋₆ aliphatic.
286. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OMe modification.
287. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification, wherein each R is optionally substituted C₁₋₆ aliphatic.
288. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification.
289. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) LNA sugars.
290. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).
291. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-F modification.
292. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) sugars comprising 2′-OH.
293. The oligonucleotide of any one of the preceding Embodiments, wherein the second domain comprises one or more (e.g., about 1-20, 1, 2, 3, 4, 5, 6, 7, 8, 9, 10, 11, 12, 13, 14, 15, 16, 17, 18, 19, 20, etc.) sugars comprising two 2′-H.
294. The oligonucleotide of any one of the preceding Embodiments, wherein the second domain comprise about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages.
295. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in the second domain are modified internucleotidic linkages.
296. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of internucleotidic linkages in the second domain are modified internucleotidic linkages.
297. The oligonucleotide of any one of the preceding Embodiments, wherein each modified internucleotidic linkages is independently a chiral internucleotidic linkage.
298. The oligonucleotide of any one of the preceding Embodiments, wherein each modified internucleotidic linkages is independently a phosphorothioate internucleotidic linkage or a non-negatively charged internucleotidic linkage.
299. The oligonucleotide of any one of the preceding Embodiments, wherein each modified internucleotidic linkages is independently a phosphorothioate internucleotidic linkage or a neutral internucleotidic linkage.
300. The oligonucleotide of any one of the preceding Embodiments, wherein the second domain comprises one or more phosphorothioate internucleotidic linkages.
301. The oligonucleotide of any one of the preceding Embodiments, wherein the second domain comprises 1, 2, 3, 4, or 5 non-negatively charged internucleotidic linkages.
302. The oligonucleotide of any one of the preceding Embodiments, wherein the internucleotidic linkage between the last and the second last nucleosides of the second domain is a non-negatively charged internucleotidic linkage.
303. The oligonucleotide of any one of the preceding Embodiments, wherein the internucleotidic linkage between the first and the second nucleosides of the second domain is a non-negatively charged internucleotidic linkage.
304. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the second domain is chirally controlled.
305. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the second domain is chirally controlled.
306. The oligonucleotide of any one of the preceding Embodiments, wherein the internucleotidic linkage between the last and the second last nucleosides of the second domain is chirally controlled.
307. The oligonucleotide of any one of the preceding Embodiments, wherein the internucleotidic linkage between the first and the second nucleosides of the second domain is a chirally controlled.
308. The oligonucleotide of any one of the preceding Embodiments, wherein each chiral internucleotidic linkage is independently a chirally controlled internucleotidic linkage.
309. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the second domain is Sp.
310. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the second domain is Sp, or wherein each chiral internucleotidic linkages in the second domain is Sp.
311. The oligonucleotide of any one of the preceding Embodiments, wherein the internucleotidic linkage between the first and the second nucleosides of the second domain is Rp.
312. The oligonucleotide of any one of the preceding Embodiments, wherein the internucleotidic linkage between the last and the second last nucleosides of the second domain is Rp.
313. The oligonucleotide of any one of the preceding Embodiments, wherein each internucleotidic linkage in the second domain is independently a modified internucleotidic linkage.
314. The oligonucleotide of any one of Embodiments 1-312, wherein the second domain comprises one or more natural phosphate linkages.
315. The oligonucleotide of any one of the preceding Embodiments, wherein the second domain can recruit, or promotes or contributes to recruitment of, an ADAR protein to a target nucleic acid.
316. The oligonucleotide of any one of the preceding Embodiments, wherein the second domain can interact, or promotes or contributes to interaction of, an ADAR protein with a target nucleic acid.
317. The oligonucleotide of any one of the preceding Embodiments, wherein the second domain contacts with a domain that have an enzymatic activity.
318. The oligonucleotide of any one of the preceding Embodiments, wherein the second domain contact with a domain that has a deaminase activity of ADAR1.
319. The oligonucleotide of any one of the preceding Embodiments, wherein the second domain contact with a domain that has a deaminase activity of ADAR2.
320. The oligonucleotide of any one of the preceding Embodiments, wherein the second domain comprises or consists of from the 5′ to 3′ a first subdomain, a second subdomain, and a third subdomain.
321. The oligonucleotide of any one of the preceding Embodiments, wherein the second domain consists of from the 5′ to 3′ a first subdomain, a second subdomain, and a third subdomain.
322. The oligonucleotide of any one of the preceding Embodiments, wherein the first subdomain has a length of about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases.
323. The oligonucleotide of any one of the preceding Embodiments, wherein the first subdomain has a length of about 10-20 (e.g., about 10, 11, 12, 13, 14, 15, 16, 17, 18, 19, or 20) nucleobases.
324. The oligonucleotide of any one of the preceding Embodiments, wherein the first subdomain comprises one or more (e.g., 1-10, 1, 2, 3, 4, 5, 6, 7, 8, 9, or 10, etc.) mismatches when the oligonucleotide is aligned with a target nucleic acid for complementarity.
325. The oligonucleotide of any one of the preceding Embodiments, wherein the first subdomain comprises two or more mismatches when the oligonucleotide is aligned with a target nucleic acid for complementarity.
326. The oligonucleotide of any one of Embodiments 1-324, wherein the first subdomain comprises one and no more than one mismatch when the oligonucleotide is aligned with a target nucleic acid for complementarity.
327. The oligonucleotide of any one of Embodiments 1-324, wherein the first subdomain comprises two and no more than two mismatches when the oligonucleotide is aligned with a target nucleic acid for complementarity.
328. The oligonucleotide of any one of the preceding Embodiments, wherein the first subdomain comprises one or more (e.g., 1-10, 1, 2, 3, 4, 5, 6, 7, 8, 9, or 10, etc.) bulges when the oligonucleotide is aligned with a target nucleic acid for complementarity.
329. The oligonucleotide of Embodiment 328, wherein each bulge independently comprises one or more base pairs that are not Watson-Crick or wobble pairs.
330. The oligonucleotide of any one of the preceding Embodiments, wherein the first subdomain comprises one or more (e.g., 1-10, 1, 2, 3, 4, 5, 6, 7, 8, 9, or 10, etc.) wobble pairs when the oligonucleotide is aligned with a target nucleic acid for complementarity.
331. The oligonucleotide of any one of the preceding Embodiments, wherein the first subdomain comprises two or more wobble pairs when the oligonucleotide is aligned with a target nucleic acid for complementarity.
332. The oligonucleotide of any one of the preceding Embodiments, wherein the first subdomain comprises two and no more than two wobble pairs when the oligonucleotide is aligned with a target nucleic acid for complementarity.
333. The oligonucleotide of any one of Embodiments 1-322, wherein the first subdomain is fully complementary to a target nucleic acid.
334. The oligonucleotide of any one of the preceding Embodiments, wherein the first subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F.
335. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in the first subdomain are independently modified sugars with a modification that is not 2′-F.
336. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the first subdomain are independently modified sugars with a modification that is not 2′-F.
337. The oligonucleotide of any one of Embodiments 334-336, wherein the first subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
338. The oligonucleotide of any one of Embodiments 334-336, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in the first subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
339. The oligonucleotide of any one of Embodiments 334-336, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the first subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
340. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification, wherein each R is optionally substituted C₁₋₆ aliphatic.
341. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification.
342. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) LNA sugars.
343. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).
344. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-F modification.
345. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising 2′-OH.
346. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising two 2′-H.
347. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OR modification, wherein R is optionally substituted C₁₋₆ aliphatic.
348. The oligonucleotide of any one of the preceding Embodiments, wherein the first subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OMe modification.
349. The oligonucleotide of any one of Embodiments 320-339, wherein each sugar in the first subdomain independently comprises a 2′-OR modification, wherein R is optionally substituted C₁₋₆ aliphatic, or a 2′-O-L^{B}-4′ modification.
350. The oligonucleotide of Embodiment 349, wherein each sugar in the first subdomain independently comprises a 2′-OR modification, wherein R is optionally substituted C₁₋₆ aliphatic, or a 2′-O-L^{B}-4′ modification, wherein L^{B} is optionally substituted —CH₂—.
351. The oligonucleotide of Embodiment 349, wherein each sugar in the first subdomain independently comprises 2′-OMe.
352. The oligonucleotide of any one of the preceding Embodiments, wherein the first subdomain comprises a 5′-end portion having a length of about 3-8 nucleobases.
353. The oligonucleotide of Embodiment 352, wherein the 5′-end portion has a length of about 3-6 nucleobases.
354. The oligonucleotide of Embodiment 352 or 353, wherein the 5′-end portion comprises the 5′-end nucleobase of the first subdomain.
355. The oligonucleotide of any one of Embodiments 352-354, wherein one or more of the sugars in the 5′-end portion are independently modified sugars.
356. The oligonucleotide of Embodiment 355, wherein the modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
357. The oligonucleotide of Embodiment 355, wherein one or more of the modified sugars independently comprises 2′-F or 2′-OR, wherein R is independently optionally substituted C₁₋₆ aliphatic.
358. The oligonucleotide of Embodiment 355, wherein one or more of the modified sugars are independently 2′-F or 2′-OMe.
359. The oligonucleotide of any one of Embodiments 352-358, wherein the 5′-end portion comprises one or more mismatches.
360. The oligonucleotide of any one of Embodiments 352-359, wherein the 5′-end portion comprises one or more wobbles.
361. The oligonucleotide of any one of Embodiments 352-360, wherein the 5′-end portion is about 60-100% (e.g., 66%, 70%, 75%, 80%, 85%, 90%, 95%, or more) complementary to a target nucleic acid.
362. The oligonucleotide of any one of the preceding Embodiments, wherein the first subdomain comprises a 3′-end portion having a length of about 3-8 nucleobases.
363. The oligonucleotide of Embodiment 362, wherein the 3′-end portion has a length of about 1-3 nucleobases.
364. The oligonucleotide of Embodiment 362 or 363, wherein the 3′-end portion comprises the 3′-end nucleobase of the first subdomain.
365. The oligonucleotide of any one of Embodiments 362-364, wherein one or more of the sugars in the 3′-end portion are independently modified sugars.
366. The oligonucleotide of Embodiment 365, wherein the modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
367. The oligonucleotide of Embodiment 365, wherein one or more of the modified sugars independently comprise 2′-F.
368. The oligonucleotide of any one of Embodiments 365-367, wherein no modified sugars comprise 2′-OMe.
369. The oligonucleotide of any one of Embodiments 362-368, wherein each sugar of the 3′-end portion independently comprises two 2′-H or a 2′-F modification.
370. The oligonucleotide of any one of Embodiments 352-358, wherein the 3′-end portion comprises one or more mismatches.
371. The oligonucleotide of any one of Embodiments 352-359, wherein the 3′-end portion comprises one or more wobbles.
372. The oligonucleotide of any one of Embodiments 352-360, wherein the 3′-end portion is about 60-100% (e.g., 66%, 70%, 75%, 80%, 85%, 90%, 95%, or more) complementary to a target nucleic acid.
373. The oligonucleotide of any one of the preceding Embodiments, wherein the first subdomain comprise about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages.
374. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in the first subdomain are modified internucleotidic linkages.
375. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of internucleotidic linkages in the first subdomain are modified internucleotidic linkages.
376. The oligonucleotide of any one of the preceding Embodiments, wherein each modified internucleotidic linkages is independently a chiral internucleotidic linkage.
377. The oligonucleotide of any one of the preceding Embodiments, wherein the internucleotidic linkage between the first and the second nucleosides of the first subdomain is a non-negatively charged internucleotidic linkage.
378. The oligonucleotide of any one of the preceding Embodiments, wherein each modified internucleotidic linkages is independently a phosphorothioate internucleotidic linkage or a non-negatively charged internucleotidic linkage.
379. The oligonucleotide of any one of the preceding Embodiments, wherein each modified internucleotidic linkages is independently a phosphorothioate internucleotidic linkage or a neutral internucleotidic linkage.
380. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the first subdomain is chirally controlled.
381. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the first subdomain is chirally controlled.
382. The oligonucleotide of any one of the preceding Embodiments, wherein the internucleotidic linkage between the first and the second nucleosides of the first subdomain is chirally controlled.
383. The oligonucleotide of any one of the preceding Embodiments, wherein each chiral internucleotidic linkage is independently a chirally controlled internucleotidic linkage.
384. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the first subdomain is Sp.
385. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the first subdomain is Sp.
386. The oligonucleotide of any one of the preceding Embodiments, wherein each chiral internucleotidic linkages in the first subdomain is Sp.
387. The oligonucleotide of any one of Embodiments 1-386, wherein the internucleotidic linkage between the first and the second nucleosides of the first subdomain is Rp.
388. The oligonucleotide of any one of the preceding Embodiments, wherein each internucleotidic linkage in the first subdomain is independently a modified internucleotidic linkage.
389. The oligonucleotide of any one of Embodiments 1-387, wherein the first subdomain comprises one or more natural phosphate linkages.
390. The oligonucleotide of any one of the preceding Embodiments, wherein the first subdomain can recruit, or promotes or contributes to recruitment of, an ADAR protein to a target nucleic acid.
391. The oligonucleotide of any one of the preceding Embodiments, wherein the first subdomain can interact, or promotes or contributes to interaction of, an ADAR protein with a target nucleic acid.
392. The oligonucleotide of any one of the preceding Embodiments, wherein the first subdomain contacts with a domain that have an enzymatic activity.
393. The oligonucleotide of any one of the preceding Embodiments, wherein the first subdomain contact with a domain that has a deaminase activity of ADAR1.
394. The oligonucleotide of any one of the preceding Embodiments, wherein the first subdomain contact with a domain that has a deaminase activity of ADAR2.
395. The oligonucleotide of any one of the preceding Embodiments, wherein the second subdomain has a length of about 1-10 (e.g., about 1, 2, 3, 4, 5, 6, 7, 8, 9, or 10) nucleobases.
396. The oligonucleotide of any one of the preceding Embodiments, wherein the second subdomain has a length of about 1-5 (e.g., about 1, 2, 3, 4, or 5) nucleobases.
397. The oligonucleotide of any one of the preceding Embodiments, wherein the second subdomain has a length of about 1, 2, or 3 nucleobases.
398. The oligonucleotide of any one of the preceding Embodiments, wherein the second subdomain has a length of 3 nucleobases.
399. The oligonucleotide of any one of the preceding Embodiments, wherein the second subdomain comprises a nucleoside opposite to a target adenosine.
400. The oligonucleotide of any one of the preceding Embodiments, wherein the second domain comprises one and no more than one nucleoside opposite to a target adenosine.
401. The oligonucleotide of any one of the preceding Embodiments, wherein the second subdomain comprises one or more (e.g., 1-10, 1, 2, 3, 4, 5, 6, 7, 8, 9, or 10, etc.) mismatches when the oligonucleotide is aligned with a target nucleic acid for complementarity.
402. The oligonucleotide of any one of the preceding Embodiments, wherein the second subdomain comprises two or more mismatches when the oligonucleotide is aligned with a target nucleic acid for complementarity.
403. The oligonucleotide of any one of Embodiments 1-401, wherein the second subdomain comprises one and no more than one mismatch when the oligonucleotide is aligned with a target nucleic acid for complementarity.
404. The oligonucleotide of any one of Embodiments 1-401, wherein the second subdomain comprises two and no more than two mismatches when the oligonucleotide is aligned with a target nucleic acid for complementarity.
405. The oligonucleotide of any one of the preceding Embodiments, wherein the second subdomain comprises one or more (e.g., 1-10, 1, 2, 3, 4, 5, 6, 7, 8, 9, or 10, etc.) bulges when the oligonucleotide is aligned with a target nucleic acid for complementarity.
406. The oligonucleotide of Embodiment 405, wherein each bulge independently comprises one or more base pairs that are not Watson-Crick or wobble pairs.
407. The oligonucleotide of any one of the preceding Embodiments, wherein the second subdomain comprises one or more (e.g., 1-10, 1, 2, 3, 4, 5, 6, 7, 8, 9, or 10, etc.) wobble pairs when the oligonucleotide is aligned with a target nucleic acid for complementarity.
408. The oligonucleotide of any one of the preceding Embodiments, wherein the second subdomain comprises two or more wobble pairs when the oligonucleotide is aligned with a target nucleic acid for complementarity.
409. The oligonucleotide of any one of the preceding Embodiments, wherein the second subdomain comprises two and no more than two wobble pairs when the oligonucleotide is aligned with a target nucleic acid for complementarity.
410. The oligonucleotide of any one of Embodiments 1-400, wherein the second subdomain is fully complementary to a target nucleic acid.
411. The oligonucleotide of any one of the preceding Embodiments, wherein the second subdomain comprises one or more sugars comprising two 2′-H (e.g., natural DNA sugars).
412. The oligonucleotide of any one of the preceding Embodiments, wherein the second subdomain comprises one or more sugars comprising 2′-OH (e.g., natural RNA sugars).
413. The oligonucleotide of any one of the preceding Embodiments, wherein the second subdomain comprises about 1-10 (e.g., 1, 2, 3, 4, 5, 6, 7, 8, 9, or 10) modified sugars.
414. The oligonucleotide of Embodiment 413, wherein each modified sugar is independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
415. The oligonucleotide of any one of the preceding Embodiments, wherein the second subdomain comprises no modified sugars comprising a 2′-OMe modification.
416. The oligonucleotide of any one of the preceding Embodiments, wherein the second subdomain comprises no modified sugars comprising a 2′-OR modification, wherein R is optionally substituted C₁₋₆ aliphatic.
417. The oligonucleotide of Embodiment 413, wherein each 2′-modified sugar is sugar comprising a 2′-F modification.
418. The oligonucleotide of any one of Embodiments 1-416, wherein the sugar of the opposite nucleoside is an acyclic sugar (e.g., a UNA sugar).
419. The oligonucleotide of any one of Embodiments 1-416, wherein the sugar of the opposite nucleoside comprises two 2′-H.
420. The oligonucleotide of any one of Embodiments 1-416, wherein the sugar of the opposite nucleoside comprises a 2′-OH.
421. The oligonucleotide of any one of Embodiments 1-416, wherein the sugar of the opposite nucleoside is a natural DNA sugar.
422. The oligonucleotide of any one of Embodiments 1-416, wherein the sugar of the opposite nucleoside comprises is modified.
423. The oligonucleotide of any one of Embodiments 1-416, wherein the sugar of the opposite nucleoside comprises 2′-F.
424. The oligonucleotide of any one of the preceding Embodiments, wherein the sugar of a nucleoside 5′-next to the opposite nucleoside (sugar of N₁ in 5′- . . . N₁N₀ . . . 3′, wherein when aligned with a target, N₀ is opposite to a target adenosine) comprises two 2′-H.
425. The oligonucleotide of any one of the preceding Embodiments, wherein the sugar of a nucleoside 5′-next to the opposite nucleoside (sugar of N₁ in 5′- . . . N₁N₀ . . . 3′, wherein when aligned with a target, N₀ is opposite to a target adenosine) comprises 2′-OH.
426. The oligonucleotide of any one of the preceding Embodiments, wherein the sugar of a nucleoside 5′-next to the opposite nucleoside (sugar of N₁ in 5′- . . . N₁N₀ . . . 3′, wherein when aligned with a target, N₀ is opposite to a target adenosine) is a natural DNA sugar.
427. The oligonucleotide of any one of the preceding Embodiments, wherein the sugar of a nucleoside 5′-next to the opposite nucleoside (sugar of N₁ in 5′- . . . N₁N₀ . . . 3′, wherein when aligned with a target, N₀ is opposite to a target adenosine) comprises 2′-F.
428. The oligonucleotide of any one of the preceding Embodiments, wherein the sugar of a nucleoside 3′-next to the opposite nucleoside (sugar of N₋₁ in 5′- . . . N₀N₋₁ . . . 3′, wherein when aligned with a target, N₀ is opposite to a target adenosine) comprises two 2′-H.
429. The oligonucleotide of any one of the preceding Embodiments, wherein the sugar of a nucleoside 3′-next to the opposite nucleoside (sugar of N₋₁ in 5′- . . . N₀N₋₁ . . . 3′, wherein when aligned with a target, N₀ is opposite to a target adenosine) comprises 2′-OH.
430. The oligonucleotide of any one of the preceding Embodiments, wherein the sugar of a nucleoside 3′-next to the opposite nucleoside (sugar of N₋₁ in 5′- . . . N₀N₋₁ . . . 3′, wherein when aligned with a target, N₀ is opposite to a target adenosine) is a natural DNA sugar.
431. The oligonucleotide of any one of the preceding Embodiments, wherein the sugar of a nucleoside 3′-next to the opposite nucleoside (sugar of N-1 in 5′- . . . N₀N₋₁ . . . 3′, wherein when aligned with a target, N₀ is opposite to a target adenosine) comprises 2′-F.
432. The oligonucleotide of any one of Embodiments 1-416, wherein each of the sugar of the opposite nucleoside, the sugar of a nucleoside 5′-next to the opposite nucleoside (sugar of N₁ in 5′- . . . N₁N₀ . . . 3′, wherein when aligned with a target, N₀ is opposite to a target adenosine), and the sugar of a nucleoside 3′-next to the opposite nucleoside (sugar of N₋₁ in 5′- . . . N₀N₋₁ . . . 3′, wherein when aligned with a target, N₀ is opposite to a target adenosine) is independently a natural DNA sugar.
433. The oligonucleotide of any one of Embodiments 1-416, wherein the sugar of the opposite nucleoside is a natural DNA sugar, the sugar of a nucleoside 5′-next to the opposite nucleoside (sugar of N₁ in 5′- . . . N₁N₀ . . . 3′, wherein when aligned with a target, N₀ is opposite to a target adenosine) is a 2′-F modified sugar, and the sugar of a nucleoside 3′-next to the opposite nucleoside (sugar of N₋₁ in 5′- . . . N₀N₋₁ . . . 3′, wherein when aligned with a target, N₀ is opposite to a target adenosine) is a natural DNA sugar.
434. The oligonucleotide of any one of the preceding Embodiments, wherein the second subdomain comprise a 5′-end portion connected to 5′-side the opposite nucleoside.
435. The oligonucleotide of Embodiment 431, wherein the 5′-end portion comprises one or more mismatches or wobbles when aligned with a target nucleic acid for complementarity.
436. The oligonucleotide of Embodiment 431 or 435, wherein the 5′-end portion has a length of 1, 2 or 3 nucleobases.
437. The oligonucleotide of and one of Embodiments 431-436, wherein sugars of the 5′-end portion are selected from sugars having two 2′-H (e.g., natural DNA sugar) and 2′-F modified sugars.
438. The oligonucleotide of any one of the preceding Embodiments, wherein the second subdomain comprise a 3′-end portion connected to the 3′-side of the opposite nucleoside.
439. The oligonucleotide of Embodiment 438, wherein the 3′-end portion comprises one or more mismatches or wobbles when aligned with a target nucleic acid for complementarity.
440. The oligonucleotide of Embodiment 438, wherein the 3′-end portion comprises one or more mismatches and/or wobbles when aligned with a target nucleic acid for complementarity.
441. The oligonucleotide of Embodiment 438, wherein the 3′-end portion comprises one or more wobbles when aligned with a target nucleic acid for complementarity.
442. The oligonucleotide of Embodiment 438, wherein the 3′-end portion comprises an I or a derivative thereof.
443. The oligonucleotide of Embodiment 438, wherein the 3′-end portion comprises an I and an I-C wobble when aligned with a target nucleic acid for complementarity.
444. The oligonucleotide of any one of Embodiments 438-443, wherein the 3′-end portion has a length of 1, 2 or 3 nucleobases.
445. The oligonucleotide of and one of Embodiments 438-444, wherein sugars of the 3′-end portion are selected from sugars having two 2′-H (e.g., natural DNA sugar) and 2′-F modified sugars.
446. The oligonucleotide of and one of Embodiments 438-444, wherein sugars of the 3′-end portion are sugars having two 2′-H (e.g., natural DNA sugar).
447. The oligonucleotide of any one of the preceding Embodiments, wherein the second subdomain comprise about 1-10 (e.g., about 1, 2, 3, 4, 5, 6, 7, 8, 9, 10, 11, 12, 13, 14, 15, 16, 17, 18, 19, or 20, etc.) modified internucleotidic linkages.
448. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in the second subdomain are modified internucleotidic linkages.
449. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of internucleotidic linkages in the second subdomain are modified internucleotidic linkages.
450. The oligonucleotide of any one of the preceding Embodiments, wherein each modified internucleotidic linkages in the second subdomain is independently a chiral internucleotidic linkage.
451. The oligonucleotide of any one of the preceding Embodiments, wherein each modified internucleotidic linkages in the second subdomain is independently a phosphorothioate internucleotidic linkage or a non-negatively charged internucleotidic linkage.
452. The oligonucleotide of any one of the preceding Embodiments, wherein each modified internucleotidic linkages in the second subdomain is independently a phosphorothioate internucleotidic linkage or a neutral internucleotidic linkage.
453. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the second subdomain is chirally controlled.
454. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the second subdomain is chirally controlled.
455. The oligonucleotide of any one of the preceding Embodiments, wherein each chiral internucleotidic linkage in the second subdomain is independently a chirally controlled internucleotidic linkage.
456. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the second subdomain is Sp.
457. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the second subdomain is Rp.
458. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the second subdomain is Sp.
459. The oligonucleotide of any one of the preceding Embodiments, wherein each chiral internucleotidic linkages in the second subdomain is Sp.
460. The oligonucleotide of any one of the preceding Embodiments, wherein each internucleotidic linkage in the second subdomain is independently a modified internucleotidic linkage.
461. The oligonucleotide of any one of Embodiments 1-459, wherein the second subdomain comprises one or more natural phosphate linkages.
462. The oligonucleotide of any one of Embodiments 1-459, wherein the opposite nucleoside is connected to its 5′ immediate nucleoside through a natural phosphate linkage.
463. The oligonucleotide of any one of Embodiments 1-461, wherein the opposite nucleoside is connected to its 5′ immediate nucleoside through a modified internucleotidic linkage.
464. The oligonucleotide of any one of Embodiments 1-463, wherein the opposite nucleoside is connected to its 3′ immediate nucleoside through a modified internucleotidic linkage.
465. The oligonucleotide of any one of Embodiments 1-464, wherein the nucleoside (position −1) that is 3′ immediate to an opposite nucleoside (position 0) is connected to its 3′ immediate nucleoside (position −2) through a modified internucleotidic linkage.
466. The oligonucleotide of any one of Embodiments 463-465, wherein the modified internucleotidic linkage is a chiral internucleotidic linkage.
467. The oligonucleotide of any one of Embodiments 463-466, wherein the modified internucleotidic linkage is a phosphorothioate internucleotidic linkage.
468. The oligonucleotide of any one of Embodiments 463-466, wherein the modified internucleotidic linkage is a non-negatively charged internucleotidic linkage.
469. The oligonucleotide of any one of Embodiments 463-466, wherein the modified internucleotidic linkage is a neutral charged internucleotidic linkage.
470. The oligonucleotide of any one of Embodiments 466-469, wherein the chiral internucleotidic linkage is chirally controlled.
471. The oligonucleotide of any one of Embodiments 466-470, wherein the chiral internucleotidic linkage is Rp.
472. The oligonucleotide of any one of Embodiments 466-470, wherein the chiral internucleotidic linkage is Sp.
473. The oligonucleotide of any one of Embodiments 462-472, wherein the 5′ immediate nucleoside comprises a modified sugar.
474. The oligonucleotide of any one of Embodiments 462-472, wherein the 5′ immediate nucleoside comprises a modified sugar comprising a 2′-F modification.
475. The oligonucleotide of any one of Embodiments 462-472, wherein the 5′ immediate nucleoside comprises a sugar comprising two 2′-H (e.g., a natural DNA sugar).
476. The oligonucleotide of any one of Embodiments 1-459 and 461-475, wherein the opposite nucleoside is connected to its 3′ immediate nucleoside through a natural phosphate linkage.
477. The oligonucleotide of any one of Embodiments 1-459 and 461-475, wherein the opposite nucleoside is connected to its 3′ immediate nucleoside through a modified internucleotidic linkage.
478. The oligonucleotide of Embodiment 477, wherein the modified internucleotidic linkage is a chiral internucleotidic linkage.
479. The oligonucleotide of Embodiment 477 or 478, wherein the modified internucleotidic linkage is a phosphorothioate internucleotidic linkage.
480. The oligonucleotide of Embodiment 477 or 478, wherein the modified internucleotidic linkage is a non-negatively charged internucleotidic linkage.
481. The oligonucleotide of Embodiment 477 or 478, wherein the modified internucleotidic linkage is a neutral charged internucleotidic linkage.
482. The oligonucleotide of any one of Embodiments 478-481, wherein the chiral internucleotidic linkage is chirally controlled.
483. The oligonucleotide of any one of Embodiments 478-482, wherein the chiral internucleotidic linkage is Rp.
484. The oligonucleotide of any one of Embodiments 478-482, wherein the chiral internucleotidic linkage is Sp.
485. The oligonucleotide of any one of the preceding Embodiments, wherein the 3′ immediate nucleoside comprises a modified sugar.
486. The oligonucleotide of Embodiment 484, wherein the 3′ immediate nucleoside comprises a modified sugar comprising a 2′-F modification.
487. The oligonucleotide of Embodiment 484, wherein the 3′ immediate nucleoside comprises a sugar comprising two 2′-H (e.g., a natural DNA sugar).
488. The oligonucleotide of any one of the preceding Embodiments, wherein the 3′-immediate nucleoside comprises a base that is not G.
489. The oligonucleotide of any one of the preceding Embodiments, wherein the 3′-immediate nucleoside comprises a base that are less steric than G.
490. The oligonucleotide of any one of the preceding Embodiments, wherein the 3′-immediate nucleoside comprises a nucleobase which is or comprise Ring BA having the structure of formula BA-VI.
491. The oligonucleotide of any one of Embodiment 488-490, wherein Ring BA is the Ring BA of any one of Embodiments 213-279.
492. The oligonucleotide of any one of Embodiment 488-491, wherein the nucleobase is

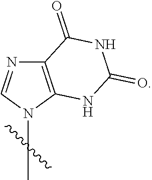 [SMILES: *n1cnc2c(=O)[nH]c(=O)[nH]c21]

493. The oligonucleotide of any one of Embodiment 488-491, wherein the nucleobase is

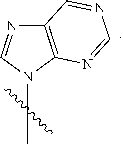 [SMILES: *n1cnc2cncnc21]

494. The oligonucleotide of any one of Embodiment 488-491, wherein the nucleobase is hypoxanthine.
495. The oligonucleotide of any one of the preceding Embodiments, wherein a target nucleic acid comprises 5′-CA-3′, wherein A is a target adenosine.
496. The oligonucleotide of any one of the preceding Embodiments, wherein the sugar in a 5′ immediate nucleoside is or comprises

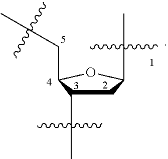

497. The oligonucleotide of any one of Embodiments 1-495, wherein the sugar in a 5′ immediate nucleoside is or comprises

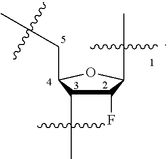

498. The oligonucleotide of any one of Embodiments 1-495, wherein the sugar in a 5′ immediate nucleoside is or comprises

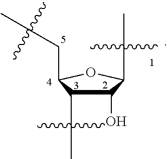

499. The oligonucleotide of any one of the preceding Embodiments, wherein the sugar in a nucleoside opposition to a target nucleoside is or comprises

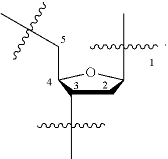

500. The oligonucleotide of any one of Embodiments 1-498, wherein the sugar in a nucleoside opposition to a target nucleoside is or comprises

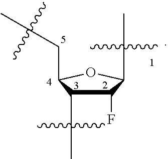

501. The oligonucleotide of any one of Embodiments 1-498, wherein the sugar in a nucleoside opposition to a target nucleoside is or comprises

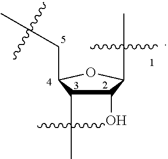

502. The oligonucleotide of any one of the preceding Embodiments, wherein the sugar in a 3′ immediate nucleoside is or comprises

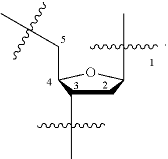

503. The oligonucleotide of any one of Embodiments 1-501, wherein the sugar in a 3′ immediate nucleoside is or comprises

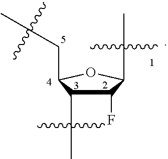

504. The oligonucleotide of any one of Embodiments 1-501, wherein the sugar in a 3′-immediate nucleoside is or comprises

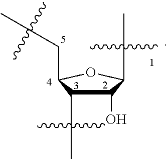

505. The oligonucleotide of any one of the preceding Embodiments, wherein the second subdomain can recruit, or promotes or contributes to recruitment of, an ADAR protein to a target nucleic acid.
506. The oligonucleotide of any one of the preceding Embodiments, wherein the second subdomain can interact, or promotes or contributes to interaction of, an ADAR protein with a target nucleic acid.
507. The oligonucleotide of any one of the preceding Embodiments, wherein the second subdomain contacts with a domain that have an enzymatic activity.
508. The oligonucleotide of any one of the preceding Embodiments, wherein the second subdomain contact with a domain that has a deaminase activity of ADAR1.
509. The oligonucleotide of any one of the preceding Embodiments, wherein the second subdomain contact with a domain that has a deaminase activity of ADAR2.
510. The oligonucleotide of any one of the preceding Embodiments, wherein the third subdomain has a length of about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc.) nucleobases.
511. The oligonucleotide of any one of the preceding Embodiments, wherein the third subdomain has a length of about 1-10 (e.g., 1, 2, 3, 4, 5, 6, 7, 8, 9, or 10) nucleobases.
512. The oligonucleotide of any one of the preceding Embodiments, wherein the third subdomain comprises one or more (e.g., 1-10, 1, 2, 3, 4, 5, 6, 7, 8, 9, or 10, etc.) mismatches when the oligonucleotide is aligned with a target nucleic acid for complementarity.
513. The oligonucleotide of any one of the preceding Embodiments, wherein the third subdomain comprises two or more mismatches when the oligonucleotide is aligned with a target nucleic acid for complementarity.
514. The oligonucleotide of any one of Embodiments 1-512, wherein the third subdomain comprises one and no more than one mismatch when the oligonucleotide is aligned with a target nucleic acid for complementarity.
515. The oligonucleotide of any one of Embodiments 1-512, wherein the third subdomain comprises two and no more than two mismatches when the oligonucleotide is aligned with a target nucleic acid for complementarity.
516. The oligonucleotide of any one of the preceding Embodiments, wherein the third subdomain comprises one or more (e.g., 1-10, 1, 2, 3, 4, 5, 6, 7, 8, 9, or 10, etc.) bulges when the oligonucleotide is aligned with a target nucleic acid for complementarity.
517. The oligonucleotide of Embodiment 516, wherein each bulge independently comprises one or more base pairs that are not Watson-Crick or wobble pairs.
518. The oligonucleotide of any one of the preceding Embodiments, wherein the third subdomain comprises one or more (e.g., 1-10, 1, 2, 3, 4, 5, 6, 7, 8, 9, or 10, etc.) wobble pairs when the oligonucleotide is aligned with a target nucleic acid for complementarity.
519. The oligonucleotide of any one of the preceding Embodiments, wherein the third subdomain comprises two or more wobble pairs when the oligonucleotide is aligned with a target nucleic acid for complementarity.
520. The oligonucleotide of any one of the preceding Embodiments, wherein the third subdomain comprises two and no more than two wobble pairs when the oligonucleotide is aligned with a target nucleic acid for complementarity.
521. The oligonucleotide of any one of Embodiments 1-511, wherein the third subdomain is fully complementary to a target nucleic acid.
522. The oligonucleotide of any one of the preceding Embodiments, wherein the third subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently with a modification that is not 2′-F.
523. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in the third subdomain are independently modified sugars with a modification that is not 2′-F.
524. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the third subdomain are independently modified sugars with a modification that is not 2′-F.
525. The oligonucleotide of any one of the preceding Embodiments, wherein the third subdomain comprises about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified sugars independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
526. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of sugars in the third subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
527. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of sugars in the third subdomain are independently modified sugars selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
528. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-N(R)₂ modification, wherein each R is optionally substituted C₁₋₆ aliphatic.
529. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-NH₂ modification.
530. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) LNA sugars.
531. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) acyclic sugars (e.g., UNA sugars).
532. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-F modification.
533. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising 2′-OH.
534. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) sugars comprising two 2′-H.
535. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OR modification, wherein R is optionally substituted C₁₋₆ aliphatic.
536. The oligonucleotide of any one of the preceding Embodiments, wherein the third subdomain comprises one or more (e.g., about 1-20, 1, 2, 3, 4, 5, 6, 7, 8, 9, 10, 11, 12, 13, 14, 15, 16, 17, 18, 19, 20, etc.) modified sugars comprising a 2′-OMe modification.
537. The oligonucleotide of any one of Embodiments 1-527, wherein each sugar in the third subdomain independently comprises a 2′-OR modification, wherein R is optionally substituted C₁₋₆ aliphatic, or a 2′-O-L^{B}-4′ modification.
538. The oligonucleotide of Embodiment 537, wherein each sugar in the third subdomain independently comprises a 2′-OR modification, wherein R is optionally substituted C₁₋₆ aliphatic, or a 2′-O-L^{B}-4′ modification, wherein L^{B} is optionally substituted —CH₂—.
539. The oligonucleotide of Embodiment 537, wherein each sugar in the third subdomain independently comprises 2′-OMe.
540. The oligonucleotide of any one of the preceding Embodiments, wherein the third subdomain comprises a 5′-end portion having a length of about 1-8 nucleobases.
541. The oligonucleotide of Embodiment 540, wherein the 5′-end portion has a length of about 1, 2, or 3 nucleobases
542. The oligonucleotide of Embodiment 540 or 541, wherein the 5′-end portion is bonded to the second subdomain.
543. The oligonucleotide of any one of Embodiments 540-542, wherein one or more of the sugars in the 5′-end portion are independently modified sugars.
544. The oligonucleotide of Embodiment 543, wherein the modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
545. The oligonucleotide of Embodiment 543, wherein one or more of the modified sugars independently comprises 2′-F.
546. The oligonucleotide of any one of Embodiments 540-542, wherein one or more sugars of the 5′-end portion independently comprise two 2′-H (e.g., natural DNA sugar).
547. The oligonucleotide of any one of Embodiments 540-546, wherein one or more sugars of the 5′-end portion independently comprise 2′-OH (e.g., natural RNA sugar).
548. The oligonucleotide of any one of Embodiments 540-542, wherein the sugars of the 5′-end portion independently comprise two 2′-H (e.g., natural DNA sugar) or a 2′-OH (e.g., natural RNA sugar).
549. The oligonucleotide of any one of Embodiments 540-542, wherein the sugars of the 5′-end portion are independently natural DNA or RNA sugars.
550. The oligonucleotide of any one of Embodiments 540-549, wherein the 5′-end portion comprises one or more mismatches.
551. The oligonucleotide of any one of Embodiments 540-550, wherein the 5′-end portion comprises one or more wobbles.
552. The oligonucleotide of any one of Embodiments 540-551, wherein the 5′-end portion is about 60-100% (e.g., 66%, 70%, 75%, 80%, 85%, 90%, 95%, or more) complementary to a target nucleic acid.
553. The oligonucleotide of any one of the preceding Embodiments, wherein the third subdomain comprises a 3′-end portion having a length of about 1-8 nucleobases.
554. The oligonucleotide of Embodiment 553, wherein the 3′-end portion has a length of about 1, 2, 3, or 4 nucleobases.
555. The oligonucleotide of Embodiment 553 or 554, wherein the 3′-end portion comprises the 3′-end nucleobase of the third subdomain.
556. The oligonucleotide of any one of Embodiments 553-555, wherein one or more of the sugars in the 3′-end portion are independently modified sugars.
557. The oligonucleotide of Embodiment 556, wherein the modified sugars are independently selected from a bicyclic sugar (e.g., a LNA sugar), an acyclic sugar (e.g., a UNA sugar), a sugar with a 2′-OR modification, or a sugar with a 2′-N(R)₂ modification, wherein each R is independently optionally substituted C₁₋₆ aliphatic.
558. The oligonucleotide of any one of Embodiments 556-557, wherein one or more modified sugars independently comprises 2′-F.
559. The oligonucleotide of any one of Embodiments 556-557, wherein at least 20%, 25%, 30%, 35%, 40%, 50%, 60%, 70%, 75%, 80%, 90%, or 95% sugars in the third subdomain independently comprise 2′-F.
560. The oligonucleotide of any one of Embodiments 556-559, wherein one or more sugars in the 3′-end portion independently comprise a 2′-OR modification, wherein R is optionally substituted C₁₋₆ aliphatic, or a 2′-O-L^{B}-4′ modification.
561. The oligonucleotide of Embodiment 560, wherein each sugar in the 3′-end portion independently comprises a 2′-OR modification, wherein R is optionally substituted C₁₋₆ aliphatic, or a 2′-O-L^{B}-4′ modification.
562. The oligonucleotide of any one of Embodiments 560-561, wherein L^{B} is optionally substituted —CH₂—.
563. The oligonucleotide of any one of Embodiments 560-561, wherein L^{B} is —CH₂—.
564. The oligonucleotide of Embodiment 560, wherein each sugar in the 3′-end portion independently comprises 2′-OMe.
565. The oligonucleotide of any one of Embodiments 553-564, wherein the 3′-end portion comprises one or more mismatches.
566. The oligonucleotide of any one of Embodiments 553-565, wherein the 3′-end portion comprises one or more wobbles.
567. The oligonucleotide of any one of Embodiments 553-566, wherein the 3′-end portion is about 60-100% (e.g., 66%, 70%, 75%, 80%, 85%, 90%, 95%, or more) complementary to a target nucleic acid.
568. The oligonucleotide of any one of the preceding Embodiments, wherein the third subdomain comprise about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) modified internucleotidic linkages.
569. The oligonucleotide of any one of the preceding Embodiments, wherein about 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of internucleotidic linkages in the third subdomain are modified internucleotidic linkages.
570. The oligonucleotide of any one of the preceding Embodiments, wherein about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 50%, 60%, 65%, 70%, 75%, 80%, 85%, 90%, 95%, or 100%, etc.) of internucleotidic linkages in the third subdomain are modified internucleotidic linkages.
571. The oligonucleotide of any one of the preceding Embodiments, wherein each modified internucleotidic linkages is independently a chiral internucleotidic linkage.
572. The oligonucleotide of any one of the preceding Embodiments, wherein the internucleotidic linkage between the last and the second last nucleosides of the third subdomain is a non-negatively charged internucleotidic linkage.
573. The oligonucleotide of any one of the preceding Embodiments, wherein each modified internucleotidic linkages is independently a phosphorothioate internucleotidic linkage or a non-negatively charged internucleotidic linkage.
574. The oligonucleotide of any one of the preceding Embodiments, wherein each modified internucleotidic linkages is independently a phosphorothioate internucleotidic linkage or a neutral internucleotidic linkage.
575. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the third subdomain is chirally controlled.
576. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the third subdomain is chirally controlled.
577. The oligonucleotide of any one of the preceding Embodiments, wherein the internucleotidic linkage between the last and the second last nucleosides of the third subdomain is chirally controlled.
578. The oligonucleotide of any one of the preceding Embodiments, wherein each chiral internucleotidic linkage is independently a chirally controlled internucleotidic linkage.
579. The oligonucleotide of any one of the preceding Embodiments, wherein at least about 1-50 (e.g., about 5, 6, 7, 8, 9, or 10-about 10, 11, 12, 13, 14, 15, 16, 17, 18, 19, 20, 21, 22, 23, 24, 25, 30, 40 or 50, or about 5, 6, 7, 8, 9, 10, 11, 12, 13, 14, 15, 16, 17, 18, 19, 20, 21, 22, 23, 24, 25, 30, 40 or 50, etc., about 1, 2, 3, 4, 5, 6, 7, 8, 9, 10, 11, 12, 13, 14, 15, 16, 17, 18, 19, or 20, etc.) chiral internucleotidic linkages in the third subdomain is Sp.
580. The oligonucleotide of any one of the preceding Embodiments, wherein at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of chiral internucleotidic linkages in the third subdomain is Sp.
581. The oligonucleotide of any one of the preceding Embodiments, wherein each chiral internucleotidic linkages in the third subdomain is Sp.
582. The oligonucleotide of any one of Embodiments 1-580, wherein the internucleotidic linkage between the last and the second lase nucleosides of the third subdomain is Rp.
583. The oligonucleotide of any one of the preceding Embodiments, wherein the internucleotidic linkage linking the last nucleoside of the second subdomain and the first nucleoside of the third subdomain is a non-negatively charged internucleotidic linkage.
584. The oligonucleotide of any one of the preceding Embodiments, wherein the internucleotidic linkage at position −2 is a non-negatively charged internucleotidic linkage.
585. The oligonucleotide of any one of Embodiments 583-584, wherein the non-negatively charged internucleotidic linkage is chirally controlled.
586. The oligonucleotide of Embodiment 585, wherein the non-negatively charged internucleotidic linkage is Rp.
587. The oligonucleotide of Embodiment 585, wherein the non-negatively charged internucleotidic linkage is Sp.
588. The oligonucleotide of any one of the preceding Embodiments, wherein each internucleotidic linkage in the third subdomain is independently a modified internucleotidic linkage.
589. The oligonucleotide of any one of Embodiments 1-587, wherein the third subdomain comprises one or more natural phosphate linkages.
590. The oligonucleotide of any one of the preceding Embodiments, wherein the third subdomain can recruit, or promotes or contributes to recruitment of, an ADAR protein to a target nucleic acid.
591. The oligonucleotide of any one of the preceding Embodiments, wherein the third subdomain can interact, or promotes or contributes to interaction of, an ADAR protein with a target nucleic acid.
592. The oligonucleotide of any one of the preceding Embodiments, wherein the third subdomain contacts with a domain that have an enzymatic activity.
593. The oligonucleotide of any one of the preceding Embodiments, wherein the third subdomain contact with a domain that has a deaminase activity of ADAR1.
594. The oligonucleotide of any one of the preceding Embodiments, wherein the third subdomain contact with a domain that has a deaminase activity of ADAR2.
595. The oligonucleotide of any one of the preceding Embodiments, wherein each wobble base pair is independently G-U, I-A, G-A, I-U, I-C, I-T, A-A, or reverse A-T.
596. The oligonucleotide of any one of the preceding Embodiments, wherein each wobble base pair is independently G-U, I-A, G-A, I-U, or I-C.
597. The oligonucleotide of any one of the preceding Embodiments, wherein each cyclic sugar or each sugar is independently optionally substituted

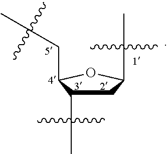

598. The oligonucleotide of any one of the preceding Embodiments, wherein each cyclic sugar or each sugar independently has the structure of

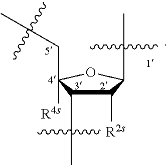

599. The oligonucleotide of Embodiment 598, wherein the oligonucleotide comprises one or more sugars wherein R^{2s} and R^{4s} are H.
600. The oligonucleotide of any one of Embodiments 598-599, wherein the oligonucleotide comprises one or more sugars wherein R^{2s} is —OR, and R^{4s} is H.
601. The oligonucleotide of any one of Embodiments 598-600, wherein the oligonucleotide comprises one or more sugars wherein R^{2s} is —OR, wherein R is optionally substituted C₁₋₄ alkyl and R^{4s} is H.
602. The oligonucleotide of any one of Embodiments 598-601, wherein the oligonucleotide comprises one or more sugars wherein R^{2s} is —OMe and R^{4s} is H.
603. The oligonucleotide of any one of Embodiments 598-602, wherein the oligonucleotide comprises one or more sugars wherein R^{2s} is —F and R^{4s} is H.
604. The oligonucleotide of any one of Embodiments 598-603, wherein the oligonucleotide comprises one or more sugars wherein R^{4s} and R^{2S} are forming a bridge having the structure of optionally substituted 2′-O—CH₂-4′.
605. The oligonucleotide of any one of Embodiments 598-603, wherein the oligonucleotide comprises one or more sugars wherein R^{4s} and R^{2S} are forming a bridge having the structure of 2′—O—CH₂-4′.
606. The oligonucleotide of any one of the preceding Embodiments, wherein the oligonucleotide comprises an additional chemical moiety.
607. The oligonucleotide of any one of the preceding Embodiments, wherein the oligonucleotide comprises a targeting moiety.
608. The oligonucleotide of any one of the preceding Embodiments, wherein the oligonucleotide comprises a carbohydrate moiety.
609. The oligonucleotide of any one of Embodiments 604-608, wherein the moiety is or comprises a ligand for an asialoglycoprotein receptor.
610. The oligonucleotide of any one of Embodiments 604-609, wherein the moiety is or comprises GalNAc or a derivative thereof 611. The oligonucleotide of any one of Embodiments 604-610, wherein the moiety is or comprises optionally substituted

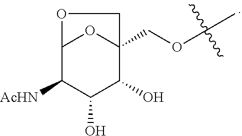 [SMILES: *OC[C@@]12COC(O1)[C@H](NC(C)=O)[C@@H](O)[C@H]2O]

612. The oligonucleotide of any one of Embodiments 604-610, wherein the moiety is or comprises optionally substituted

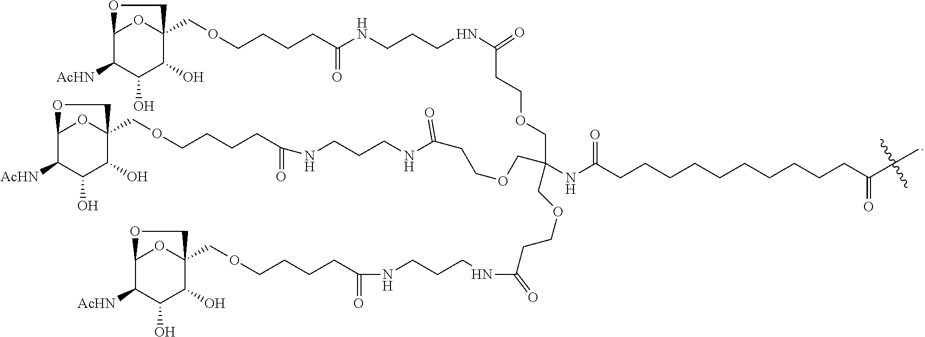 [SMILES: *C(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O]

613. The oligonucleotide of any one of Embodiments 604-612, wherein the moiety is connected to an oligonucleotide chain through a linker.
614. The oligonucleotide of Embodiment 613, wherein the linker is or comprises L001.
615. The oligonucleotide of Embodiment 499, wherein L001 is connected to 5′-end 5′-carbon of an oligonucleotide chain through a phosphate group.
616. The oligonucleotide of any one of the preceding Embodiments, wherein the oligonucleotide is in a salt form.
617. The oligonucleotide of any one of the preceding Embodiments, wherein the oligonucleotide is in a pharmaceutically acceptable salt form.
618. The oligonucleotide of any one of the preceding Embodiments, wherein the oligonucleotide is in a sodium salt form.
619. The oligonucleotide of any one of the preceding Embodiments, wherein the oligonucleotide is in an ammonium salt form.
620. The oligonucleotide of any one of the preceding Embodiments, wherein if any, at least one or each neutral internucleotidic linkage is independently n001.
621. The oligonucleotide of any one of the preceding Embodiments, wherein if any, each non-negatively charged internucleotidic linkage is independently n001.
622. The oligonucleotide of any one of the preceding Embodiments, wherein no more than 5, 6, 7, 8, 9, 10, 11 or 12 nucleosides 3′ to a nucleoside opposite to a target adenosine.
623. The oligonucleotide of any one of the preceding Embodiments, wherein no more than 5, 6, 7, 8, 9, 10, 11 or 12 nucleosides 3′ to a nucleoside opposite to a target nucleoside, wherein each of the nucleosides is independently optionally substituted A, T, C, G, U, or a tautomer thereof.
624. The oligonucleotide of any one of the preceding Embodiments, wherein about 50%-100% (e.g., about or at least about 50%, 55%, 60%, 65%, 70%, 75%, 80%, 85%, 90%, 95%) of internucleotidic linkages 3′ to a nucleoside opposite to a target adenosine are each independently a modified internucleotidic linkage.
625. The oligonucleotide of any one of the preceding Embodiments, wherein about 50%-100% (e.g., about or at least about 50%, 55%, 60%, 65%, 70%, 75%, 80%, 85%, 90%, 95%) of internucleotidic linkages 3′ to a nucleoside opposite to a target adenosine are each independently a phosphorothioate internucleotidic linkage or a non-negatively charged internucleotidic linkage.
626. The oligonucleotide of any one of the preceding Embodiments, wherein no more than 1, 2, or 3 internucleotidic linkages 3′ to a nucleoside opposite to a target adenosine are natural phosphate linkages.
627. The oligonucleotide of any one of the preceding Embodiments, wherein no more than 1, 2, or 3 internucleotidic linkages 3′ to a nucleoside opposite to a target adenosine are Rp internucleotidic linkages.
628. The oligonucleotide of any one of the preceding Embodiments, wherein no more than 1, 2, or 3 internucleotidic linkages 3′ to a nucleoside opposite to a target adenosine are Rp phosphorothioate internucleotidic linkages.
629. The oligonucleotide of any one of the preceding Embodiments, wherein the internucleotidic linkage between a nucleoside opposite to a target nucleoside and its 3′ immediate nucleoside (considered a -1 position) is a stereorandom phosphorothioate internucleotidic linkage.
630. The oligonucleotide of any one of Embodiments 1-628, wherein the internucleotidic linkage between a nucleoside opposite to a target nucleoside and its 3′ immediate nucleoside (considered a −1 position) is a chirally controlled Rp phosphorothioate internucleotidic linkage.
631. The oligonucleotide of any one of Embodiments 1-628, wherein the internucleotidic linkage between a nucleoside opposite to a target nucleoside and its 3′ immediate nucleoside (considered a −1 position) is a chirally controlled Sp phosphorothioate internucleotidic linkage
632. The oligonucleotide of any one of Embodiments 1-628, wherein an internucleotidic linkage bonded to a nucleoside opposite to a target nucleoside at the 3′-position of its sugar (considered a −1 position) is a Rp phosphorothioate internucleotidic linkage, and optionally the only Rp phosphorothioate internucleotidic linkage 3′ to a nucleoside opposite to a target adenosine.
633. The oligonucleotide of any one of Embodiments 1-628, wherein an internucleotidic linkage bonded to a nucleoside opposite to a target nucleoside at the 3′-position of its sugar (considered a −1 position) is a Sp phosphorothioate internucleotidic linkage.
634. The oligonucleotide of any one of Embodiments 1-628, wherein an internucleotidic linkage bonded to a nucleoside opposite to a target nucleoside at the 3′-position of its sugar (considered a −1 position) is a stereorandom phosphorothioate internucleotidic linkage.
635. The oligonucleotide of any one of Embodiments 1-628, wherein the internucleotidic linkage between a 3′ immediate nucleoside of a nucleoside opposite to a target nucleoside and the next 3′ immediate nucleoside (e.g., position −2 between N₋₁ and N₋₂ of 5′- . . . N₀N₋₁N₋₂ . . . -3′ wherein N₀ represents a nucleoside opposite to a target nucleoside) is a non-negatively charged internucleotidic linkage.
636. The oligonucleotide of Embodiment 635, wherein the non-negatively charged internucleotidic linkage is stereorandom.
637. The oligonucleotide of Embodiment 635, wherein the non-negatively charged internucleotidic linkage is chirally controlled.
638. The oligonucleotide of Embodiment 635, wherein the non-negatively charged internucleotidic linkage is chirally controlled and is Sp.
639. The oligonucleotide of Embodiment 635, wherein the non-negatively charged internucleotidic linkage is chirally controlled and is Rp.
640. The oligonucleotide of any one of Embodiments 635-639, wherein a non-negatively charged internucleotidic linkage is n001.
641. The oligonucleotide of any one of the preceding Embodiments, wherein the first internucleotidic linkage is a non-negatively charged internucleotidic linkage.
642. The oligonucleotide of Embodiment 641, wherein the non-negatively charged internucleotidic linkage is stereorandom.
643. The oligonucleotide of Embodiment 641, wherein the non-negatively charged internucleotidic linkage is chirally controlled.
644. The oligonucleotide of Embodiment 641, wherein the non-negatively charged internucleotidic linkage is chirally controlled and is Sp.
645. The oligonucleotide of Embodiment 641, wherein the non-negatively charged internucleotidic linkage is chirally controlled and is Rp.
646. The oligonucleotide of any one of Embodiments 641-645, wherein a non-negatively charged internucleotidic linkage is n001.
647. The oligonucleotide of any one of the preceding Embodiments, wherein the last internucleotidic linkage is a non-negatively charged internucleotidic linkage.
648. The oligonucleotide of Embodiment 647, wherein the non-negatively charged internucleotidic linkage is stereorandom.
649. The oligonucleotide of Embodiment 647, wherein the non-negatively charged internucleotidic linkage is chirally controlled.
650. The oligonucleotide of Embodiment 647, wherein the non-negatively charged internucleotidic linkage is chirally controlled and is Sp.
651. The oligonucleotide of Embodiment 647, wherein the non-negatively charged internucleotidic linkage is chirally controlled and is Rp.
652. The oligonucleotide of any one of Embodiments 647-651, wherein a non-negatively charged internucleotidic linkage is n001.
653. The oligonucleotide of any one of the preceding Embodiments, wherein an internucleotidic linkage at position −3 relative to a nucleoside opposite to a target adenosine is not a Rp phosphorothioate internucleotidic linkage.
654. The oligonucleotide of any one of the preceding Embodiments, wherein an internucleotidic linkage at position −6 relative to a nucleoside opposite to a target adenosine is not a Rp phosphorothioate internucleotidic linkage.
655. The oligonucleotide of any one of the preceding Embodiments, wherein an internucleotidic linkage at position −4 and/or −5 relative to a nucleoside opposite to a target nucleoside is a modified internucleotidic linkage, e.g., a phosphorothioate internucleotidic linkage.
656. The oligonucleotide of any one of the preceding Embodiments, wherein a nucleoside opposite to a target nucleoside is at position 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more from the 5′-end.
657. The oligonucleotide of any one of the preceding Embodiments, wherein a nucleoside opposite to a target nucleoside is at position 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more from the 3′-end.
658. The oligonucleotide of Embodiment 656 or 657, wherein the position is position 4.
659. The oligonucleotide of Embodiment 656 or 657, wherein the position is position 5.
660. The oligonucleotide of Embodiment 656 or 657, wherein the position is position 6.
661. The oligonucleotide of Embodiment 656 or 657, wherein the position is position 7.
662. The oligonucleotide of Embodiment 656 or 657, wherein the position is position 8.
663. The oligonucleotide of Embodiment 656 or 657, wherein the position is position 9.
664. The oligonucleotide of Embodiment 656 or 657, wherein the position is position 10.
665. The oligonucleotide of any one of the preceding Embodiments, about 50%-100% (e.g., about or at least about 50%, 55%, 60%, 65%, 70%, 75%, 80%, 85%, 90%, or 95%) of internucleotidic linkages 5′ to a nucleoside opposite to a target adenosine are each independently a modified internucleotidic linkage, which is optionally chirally controlled.
666. The oligonucleotide of any one of the preceding Embodiments, about 50%-100% (e.g., about or at least about 50%, 55%, 60%, 65%, 70%, 75%, 80%, 85%, 90%, or 95%) of phosphorothioate internucleotidic linkages 5′ to a nucleoside opposite to a target nucleoside (e.g., a target adenosine) are each chirally controlled and are Sp.
667. The oligonucleotide of any one of the preceding Embodiments, wherein no or no more than 1, 2, or 3 internucleotidic linkages 5′ to a nucleoside opposite to a target adenosine are natural phosphate linkages
668. The oligonucleotide of any one of the preceding Embodiments, an internucleotidic linkage at position +5 relative to a nucleoside opposite to a target nucleoside (e.g., for . . . N₊₅N₊₄N₊₃N₊₂N₊₁N₀ . . . , the internucleotidic linkage linking N₊₄ and N₊₅ wherein N₀ is a nucleoside opposite to a target nucleoside) is not a Rp phosphorothioate internucleotidic linkage.
669. The oligonucleotide of any one of the preceding Embodiments, wherein one or more or all internucleotidic linkages at positions +6 to +8 relative to a nucleoside opposite to a target adenosine are each independently a modified internucleotidic linkage, optionally chirally controlled.
670. The oligonucleotide of any one of the preceding Embodiments, wherein one or more or all internucleotidic linkages at positions +6 to +8 relative to a nucleoside opposite to a target adenosine are each independently a phosphorothioate internucleotidic linkage, optionally chirally controlled.
671. The oligonucleotide of any one of the preceding Embodiments, wherein one or more or all internucleotidic linkages at positions +6, +7, +8, +9, and +11 relative to a nucleoside opposite to a target adenosine are each independently Rp phosphorothioate internucleotidic linkages.
672. The oligonucleotide of any one of the preceding Embodiments, wherein one or more or all internucleotidic linkages at positions +5, +6, +7, +8, and +9 relative to a nucleoside opposite to a target adenosine are each independently Sp phosphorothioate internucleotidic linkages.
673. The oligonucleotide of any one of the preceding Embodiments, wherein the oligonucleotide has a complementarity of about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, or at least about 50%, 60%, 65%, 70%, 75%, 80%, 85%, 90%, 95%, or 100%, etc.) to a PiZZ allele (e.g., atcgacAagaaagggactgaagc).
674. The oligonucleotide of any one of the preceding Embodiments, wherein the oligonucleotide has a purity of about 10%-100% (e.g., about 10%-95%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, or about or at least about 10%, 20%, 30%, 40%, 50%, 60%, 65%, 70%, 75%, 80%, 85%, 90%, 95%, or 100%, etc.).
675. The oligonucleotide of any one of the preceding Embodiments, wherein the oligonucleotide has a purity of about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, or at least about 50%, 60%, 65%, 70%, 75%, 80%, 85%, 90%, 95%, or 100%, etc.).
676. A pharmaceutical composition which comprises or delivers an effective amount of an oligonucleotide of any one of the preceding Embodiments or a pharmaceutically acceptable salt thereof and a pharmaceutically acceptable carrier.
677. An oligonucleotide composition comprising a plurality of oligonucleotides, wherein oligonucleotides of the plurality share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein each oligonucleotide of the plurality is independently an oligonucleotide of any one of the preceding Embodiments or an acid, base, or salt form thereof.

678. An oligonucleotide composition comprising a plurality of oligonucleotides, wherein oligonucleotides of the plurality share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein each oligonucleotide of the plurality is independently an oligonucleotide of any one of Embodiments 780-803, or an acid, base, or salt form thereof.

679. An oligonucleotide composition comprising a plurality of oligonucleotides, wherein oligonucleotides of the plurality share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein the common base sequence is complementary to a base sequence of a portion of a nucleic acid which portion comprises a target adenosine.

680. The composition of Embodiment 679, wherein the common base sequence is complementary to a base sequence of a portion of a nucleic acid with 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) mismatches which are not Watson-Crick base pairs.
681. The composition of Embodiment 679, wherein the common base sequence is complementary to a base sequence of a portion of a nucleic acid with 0-5 mismatches which are not Watson-Crick base pairs.
682. The composition of Embodiment 679, wherein the common base sequence is 100% complementary to a base sequence of a portion of a nucleic acid across the length of the common base sequence except the nucleoside opposite to a target adenosine.
683. The composition of Embodiment 679, wherein the common base sequence is 100% complementary to a base sequence of a portion of a nucleic acid across the length of the common base sequence.
684. The composition of any one of Embodiments 677-683, wherein the composition can edit a target A to I when contacted with a nucleic acid in a system expressing ADAR.
685. The composition of any one of Embodiments 677-684, wherein the target adenosine is a G to A mutation associated with a condition, disorder or disease.
686. The composition of any one of Embodiments 677-685, wherein oligonucleotides of the plurality share the same base and sugar modifications.
687. The composition of any one of Embodiments 677-686, wherein oligonucleotides of the plurality share the same pattern of backbone chiral centers.
688. The composition of any one of Embodiments 677-687, wherein the composition is enriched for oligonucleotides of the plurality compared to a stereorandom preparation of the oligonucleotides wherein no internucleotidic linkages are chirally controlled.
689. The composition of any one of Embodiments 677-687, wherein a non-random level of all oligonucleotides in the composition that share the common base sequence and the same base and sugar modifications are oligonucleotides of the plurality.
690. The composition of any one of Embodiments 677-687, wherein a non-random level of all oligonucleotides in the composition that share the common base sequence are oligonucleotides of the plurality.
691. The composition of any one of Embodiments 677-690, wherein oligonucleotides of the plurality are of the same oligonucleotide or one or more pharmaceutically acceptable salts thereof.
692. The composition of any one of Embodiments 677-690, wherein oligonucleotides of the plurality are one or more pharmaceutically acceptable salts of the same acid-form oligonucleotide.
693. The composition of any one of Embodiments 677-690, wherein oligonucleotides of the plurality are of the same constitution.
694. The composition of Embodiment 693, wherein a non-random level of all oligonucleotides in the composition that share the same base sequence as oligonucleotides of the plurality are oligonucleotides of the plurality.
695. The composition of Embodiment 693, wherein a non-random level of all oligonucleotides in the composition that share the same constitution are oligonucleotides of the plurality.
696. The composition of any one of Embodiments 677-690, wherein oligonucleotides of the plurality are of the same structure.
697. The composition of any one of Embodiments 677-696, wherein oligonucleotides of the plurality are sodium salts.
698. The composition of any one of Embodiments 677-697, wherein oligonucleotides of the plurality share the same linkage phosphorus stereochemistry at 10 or more chiral internucleotidic linkages.
699. The composition of any one of Embodiments 677-698, wherein oligonucleotides of the plurality share the same linkage phosphorus stereochemistry at each phosphorothioate internucleotidic linkages.
700. The composition of any one of Embodiments 677-699, wherein oligonucleotides of the plurality do not share the same linkage phosphorus stereochemistry at one or more or any non-negatively charged internucleotidic linkages.
701. An oligonucleotide composition comprising one or more pluralities of oligonucleotides, wherein oligonucleotides of each plurality independently share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein each oligonucleotide of the plurality is independently an oligonucleotide of any one of the preceding Embodiments or an acid, base, or salt form thereof.

702. An oligonucleotide composition comprising one or more pluralities of oligonucleotides, wherein oligonucleotides of each plurality independently share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein each oligonucleotide of the plurality is independently an oligonucleotide of any one of the preceding Embodiments and Embodiments 780-803, or an acid, base, or salt form thereof.

703. An oligonucleotide composition comprising one or more pluralities of oligonucleotides, wherein oligonucleotides of each plurality independently share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein each oligonucleotide of the plurality is independently an oligonucleotide of any one of Embodiments 780-803, or an acid, base, or salt form thereof.

704. An oligonucleotide composition comprising one or more pluralities of oligonucleotides, wherein oligonucleotides of each plurality independently share:

1) a common base sequence, and

2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);

wherein the common base sequence of each plurality is independently complementary to a base sequence of a portion of a nucleic acid which portion comprises a target adenosine.

705. The composition of Embodiment, wherein the common base sequence is complementary to a base sequence of a portion of a nucleic acid with 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) mismatches which are not Watson-Crick base pairs.
706. The composition of Embodiment 705, wherein the common base sequence of each plurality is independently complementary to a base sequence of a portion of a nucleic acid with 0-5 mismatches which are not Watson-Crick base pairs.
707. The composition of Embodiment 705, wherein the common base sequence of each plurality is independently 100% complementary to a base sequence of a portion of a nucleic acid across the length of the common base sequence except the nucleoside opposite to a target adenosine.
708. The composition of Embodiment 705, wherein the common base sequence of each plurality is independently 100% complementary to a base sequence of a portion of a nucleic acid across the length of the common base sequence.
709. The composition of any one of Embodiments 701-708, wherein each plurality of oligonucleotides can independently edit a target A to I when contacted with a nucleic acid in a system expressing ADAR.
710. The composition of any one of Embodiments 701-709, wherein a target adenosine is a G to A mutation associated with a condition, disorder or disease.
711. The composition of any one of Embodiments 701-710, wherein the composition comprises two or more (e.g., 2, 3, 4, 5, 6, 7, 8, 9, 10, or more) pluralities of oligonucleotides.
712. The composition of any one of Embodiments 701-711, wherein common base sequences of at least two pluralities are different.
713. The composition of any one of Embodiments 701-712, wherein no two pluralities of oligonucleotides share the same common base sequence.
714. The composition of any one of Embodiments 701-713, wherein at least two pluralities of oligonucleotides target different adenosine.
715. The composition of any one of Embodiments 701-714, wherein no two pluralities of oligonucleotides target the same adenosine.
716. The composition of any one of Embodiments 701-715, wherein at least two pluralities of oligonucleotides target different transcripts.
717. The composition of any one of Embodiments 701-716, wherein no two pluralities of oligonucleotides target the same transcript.
718. The composition of any one of Embodiments 701-717, wherein at least two plurality of oligonucleotides target adenosine residues in transcripts from different polynucleotides.
719. The composition of any one of Embodiments 701-720, wherein no two pluralities of oligonucleotides target transcripts from the same polynucleotide.
720. The composition of any one of Embodiments 701-719, wherein at least two plurality of oligonucleotides target adenosine residues in transcripts from different genes.
721. The composition of any one of Embodiments 701-720, wherein no two pluralities of oligonucleotides target transcripts from the same gene.
722. The composition of any one of Embodiments 701-721, wherein oligonucleotides of each plurality independently share the same base and sugar modifications within the plurality.
723. The composition of any one of Embodiments 701-722, wherein oligonucleotides of each plurality independently share the same pattern of backbone chiral centers within the plurality.
724. The composition of any one of Embodiments 701-723, wherein for each plurality independently, the composition is enriched for oligonucleotides of that plurality compared to a stereorandom preparation of oligonucleotides of that plurality wherein no internucleotidic linkages are chirally controlled.
725. The composition of any one of Embodiments 701-724, wherein for each plurality independently, a non-random level of all oligonucleotides in the composition that share the common base sequence and the same base and sugar modifications are oligonucleotides of the plurality.
726. The composition of any one of Embodiments 701-724, wherein for each plurality independently, a non-random level of all oligonucleotides in the composition that share the common base sequence are oligonucleotides of the plurality.
727. The composition of any one of Embodiments 701-726, wherein for each plurality independently, oligonucleotides of the plurality are of the same oligonucleotide or one or more pharmaceutically acceptable salts thereof.
728. The composition of any one of Embodiments 701-727, wherein for each plurality independently, oligonucleotides of the plurality are one or more pharmaceutically acceptable salts of the same acid-form oligonucleotide.
729. The composition of any one of Embodiments 701-726, wherein for each plurality independently, oligonucleotides of the plurality are of the same constitution.
730. The composition of Embodiment 729, wherein for each plurality independently, a non-random level of all oligonucleotides in the composition that share the same base sequence as oligonucleotides of the plurality are oligonucleotides of the plurality.
731. The composition of Embodiment 729, wherein for each plurality independently, a non-random level of all oligonucleotides in the composition that share the same constitution are oligonucleotides of the plurality.
732. The composition of any one of Embodiments 701-731, wherein for one or two or all pluralities independently, oligonucleotides of the plurality are of the same structure.
733. The composition of any one of Embodiments 701-732, wherein for one or two or all pluralities independently, oligonucleotides of the plurality are each independently a pharmaceutically acceptable salt form.
734. The composition of any one of Embodiments 701-732, wherein for one or two or all pluralities independently, oligonucleotides of the plurality are sodium salts.
735. The composition of any one of Embodiments 701-734, wherein for one or two or all pluralities independently, oligonucleotides of the plurality share the same linkage phosphorus stereochemistry at 10 or more chiral internucleotidic linkages.
736. The composition of any one of Embodiments 701-735, wherein for each plurality independently, oligonucleotides of the plurality share the same linkage phosphorus stereochemistry at 10 or more chiral internucleotidic linkages.
737. The composition of any one of Embodiments 701-736, wherein for one or two or all pluralities independently, oligonucleotides of the plurality share the same linkage phosphorus stereochemistry at each phosphorothioate internucleotidic linkages.
738. The composition of any one of Embodiments 701-737, wherein for each plurality independently, oligonucleotides of the plurality share the same linkage phosphorus stereochemistry at each phosphorothioate internucleotidic linkages.
739. The composition of any one of Embodiments 701-738, wherein for one or two or all pluralities independently, oligonucleotides of the plurality do not share the same linkage phosphorus stereochemistry at one or more or any non-negatively charged internucleotidic linkages.
740. The composition of any one of Embodiments 701-739, wherein for each plurality independently, oligonucleotides of the plurality do not share the same linkage phosphorus stereochemistry at one or more or any non-negatively charged internucleotidic linkages.
741. A composition comprising a plurality of oligonucleotides which are of a particular oligonucleotide type characterized by:

a) a common base sequence;

b) a common pattern of backbone linkages;

c) a common pattern of backbone chiral centers;

d) a common pattern of backbone phosphorus modifications;

which composition is chirally controlled in that it is enriched, relative to a substantially racemic preparation of oligonucleotides having the same common base sequence, pattern of backbone linkages and pattern of backbone phosphorus modifications, for oligonucleotides of the particular oligonucleotide type, or a non-random level of all oligonucleotides in the composition that share the common base sequence are oligonucleotides of the plurality; and

wherein each oligonucleotide of the plurality is independently an oligonucleotide of any one of the preceding Embodiments or an acid, base, or salt form thereof.

742. A composition comprising a plurality of oligonucleotides which are of a particular oligonucleotide type characterized by:

a) a common base sequence;

b) a common pattern of backbone linkages;

c) a common pattern of backbone chiral centers;

d) a common pattern of backbone phosphorus modifications;

which composition is chirally controlled in that it is enriched, relative to a substantially racemic preparation of oligonucleotides having the same common base sequence, pattern of backbone linkages and pattern of backbone phosphorus modifications, for oligonucleotides of the particular oligonucleotide type, or a non-random level of all oligonucleotides in the composition that share the common base sequence are oligonucleotides of the plurality; and

wherein each oligonucleotide of the plurality is independently an oligonucleotide of any one of Embodiments 780-803, or an acid, base, or salt form thereof.

743. A composition comprising a plurality of oligonucleotides which are of a particular oligonucleotide type characterized by:

a) a common base sequence;

b) a common pattern of backbone linkages;

c) a common pattern of backbone chiral centers;

d) a common pattern of backbone phosphorus modifications;

which composition is chirally controlled in that it is enriched, relative to a substantially racemic preparation of oligonucleotides having the same common base sequence, pattern of backbone linkages and pattern of backbone phosphorus modifications, for oligonucleotides of the particular oligonucleotide type, or a non-random level of all oligonucleotides in the composition that share the common base sequence are oligonucleotides of the plurality; and

wherein the common base sequence is complementary to a base sequence of a portion of a nucleic acid which portion comprises a target adenosine.

744. The composition of Embodiment 743, wherein the common base sequence is complementary to a base sequence of a portion of a nucleic acid with 0-10 (e.g., 0-1, 0-2, 0-3, 0-4, 0-5, 0-6, 0-7, 0-8, 0-9, 0-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 0, 1, 2, 3, 4, 5, 6, 7, 8, 9, or 10, etc.) mismatches which are not Watson-Crick base pairs.
745. The composition of Embodiment 743, wherein the common base sequence is complementary to a base sequence of a portion of a nucleic acid with 0-5 mismatches which are not Watson-Crick base pairs.
746. The composition of Embodiment 743, wherein the common base sequence is 100% complementary to a base sequence of a portion of a nucleic acid across the length of the common base sequence except the nucleoside opposite to a target adenosine.
747. The composition of Embodiment 743, wherein the common base sequence is 100% complementary to a base sequence of a portion of a nucleic acid across the length of the common base sequence.
748. The composition of any one of Embodiments 741-747, wherein the composition can edit a target A to I when contacted with a nucleic acid in a system expressing ADAR.
749. The composition of any one of Embodiments 741-748, wherein the target adenosine is a G to A mutation associated with a condition, disorder or disease.
750. The composition of any one of Embodiments 741-749, wherein the composition is enriched, relative to a substantially racemic preparation of oligonucleotides having the same common base sequence, pattern of backbone linkages and pattern of backbone phosphorus modifications, for oligonucleotides of the particular oligonucleotide type.
751. The composition of any one of Embodiments 741-750, wherein a non-random level of all oligonucleotides in the composition that share the common base sequence are oligonucleotides of the plurality.
752. The composition of any one of Embodiments 677-751, wherein the level of oligonucleotides of a plurality in oligonucleotides in the composition that share the common base sequence of the plurality is about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chirally controlled internucleotidic linkages.
753. The composition of any one of Embodiments 677-751, wherein for each plurality of oligonucleotides, the level of oligonucleotides of the plurality in oligonucleotides in the composition that share the common base sequence of the plurality is independently about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chirally controlled internucleotidic linkages.
754. The composition of any one of Embodiments 677-751, wherein the level of oligonucleotides of a plurality in oligonucleotides in the composition that share the common constitution of the plurality is about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chirally controlled internucleotidic linkages.
755. The composition of any one of Embodiments 677-751, wherein for each plurality of oligonucleotides, the level of oligonucleotides of the plurality in oligonucleotides in the composition that share the common constitution of the plurality is independently about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chirally controlled internucleotidic linkages.
756. The composition of any one of Embodiments 677-755, wherein DS is about 90%-100% (e.g., about 90%, 91%, 92%, 93%, 94%, 95%, 96%, 97%, 98%, 99% or 99.5% or more).
757. The composition of any one of Embodiments 752-756, wherein nc is about 5-40 (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40) or more.
758. The composition of any one of Embodiments 677-751, wherein the level is at least about 10%-100%, or at least about 10%, 20%, 30%, 40%, 50%, 60%, 65%, 70%, 75%, 80%, 85%, 90%, or 95%.
759. The composition of any one of Embodiments 677-751, wherein the level is at least about 50%-100%, or at least about 50%, 60%, 65%, 70%, 75%, 80%, 85%, 90%, or 95%.
760. The composition of any one of Embodiments 677-759, wherein when the composition is contacted with a sample comprising the target nucleic acid and an adenosine deaminase, the target adenosine residue is modified.
761. The composition of Embodiment 760, wherein the modification is or comprises modification performed by ADAR1.
762. The composition of Embodiment 760 or 761, wherein the modification is or comprises modification performed by ADAR2.
763. The composition of any one of Embodiments 760-762, wherein the modification is performed in vitro.
764. The composition of any one of Embodiments 760-762, wherein the sample is a cell.
765. The composition of any one of Embodiments 760-764, wherein the target adenosine is converted into inosine.
766. The composition of any one of Embodiments 760-765, wherein the target adenosine is modified to a greater degree than that is observed with a comparable reference oligonucleotide composition.
767. The composition of Embodiment 766, wherein the reference oligonucleotide composition comprises no or a lower level of oligonucleotides of the plurality.
768. The composition of any one of Embodiments 766-767, wherein the reference composition does not contain oligonucleotides that have the same constitution as an oligonucleotide of the plurality.
769. The composition of any one of Embodiments 766-768, wherein the reference composition does not contain oligonucleotides that have the same structure as an oligonucleotide of the plurality.
770. The composition of Embodiment 766, wherein the reference oligonucleotide composition is a composition whose oligonucleotides having the same base sequence as oligonucleotides of the plurality contain a lower level of 2′-F modifications compared to oligonucleotides of the plurality.
771. The composition of any one of Embodiments 766-770, wherein the reference oligonucleotide composition is a composition whose oligonucleotides having the same base sequence as oligonucleotides of the plurality contain a lower level of 2′-OMe modifications compared to oligonucleotides of the plurality.
772. The composition of any one of Embodiments 766-771, wherein the reference oligonucleotide composition is a composition whose oligonucleotides having the same base sequence as oligonucleotides of the plurality have a different sugar modification pattern compared to oligonucleotides of the plurality.
773. The composition of any one of Embodiments 766-772, wherein the reference oligonucleotide composition is a composition whose oligonucleotides having the same base sequence as oligonucleotides of the plurality contain a lower level of modified internucleotidic linkages compared to oligonucleotides of the plurality.
774. The composition of any one of Embodiments 766-773, wherein the reference oligonucleotide composition is a composition whose oligonucleotides having the same base sequence as oligonucleotides of the plurality contain a lower level of phosphorothioate internucleotidic linkages compared to oligonucleotides of the plurality.
775. The composition of any one of Embodiments 766-774, wherein the reference composition is a stereorandom oligonucleotide composition.
776. The composition of Embodiment 766, wherein the reference composition is a stereorandom oligonucleotide composition of oligonucleotides of the same constitution as oligonucleotides of the plurality.
777. The composition of any one of the preceding Embodiments, wherein the composition does not cause significant degradation of the nucleic acid (e.g., no more than about 5%-100% (e.g., no more than about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.)).
778. The composition of any one of the preceding Embodiments, wherein the composition does not cause significant exon skipping or altered exon inclusion in the target nucleic acid (e.g., no more than about 5%-100% (e.g., no more than about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.)).
779. The composition of any one of Embodiments 677-778, wherein the composition is a pharmaceutical composition, and further comprises a pharmaceutically acceptable carrier.
780. An oligonucleotide, wherein the oligonucleotide is otherwise identical to an oligonucleotide of any one of the preceding Embodiments, except that at a position of a modified internucleotidic linkage is a linkage having the structure of —O⁵—P^{L}(R^{CA})—O³, wherein:

P^{L} is P, or P(═W);

W is O, S, or W^{N};

R^{CA} is or comprises an optionally substituted or capped chiral auxiliary moiety,

O⁵ is an oxygen bonded to a 5′-carbon of a sugar, and

O³ is an oxygen bonded to a 3′-carbon of a sugar.

781. The oligonucleotide of Embodiment 780, wherein the chiral auxiliary is removed the linkage is converted to the modified internucleotidic linkage.
782. The oligonucleotide of Embodiment 780, wherein a modified internucleotidic linkage is a phosphorothioate internucleotidic linkage.
783. The oligonucleotide of Embodiment 782, wherein when W is replaced with —SH and R^{CA} is replaced with O, P^{L} has the same configuration as the linkage phosphorus of the phosphorothioate internucleotidic linkage.
784. The oligonucleotide of any one of Embodiments 780-783, wherein a modified internucleotidic linkage is a neutral internucleotidic linkage.
785. The oligonucleotide of any one of Embodiments 780-783, wherein a modified internucleotidic linkage is n001.
786. The oligonucleotide of any one of Embodiments 780-785, wherein at each position of a phosphorothioate internucleotidic linkage is independently a linkage having the structure of —O⁵—P^{L}(W)(R^{CA})—O³—.
787. The oligonucleotide of any one of Embodiments 780-785, wherein at each position of a modified internucleotidic linkage is independently a linkage having the structure of —O⁵—P^{L}(W)(R^{CA})—O³—.
788. The oligonucleotide of any one of Embodiments 780-787, wherein one or each W is S.
789. The oligonucleotide of any one of Embodiments 780-788, wherein one and only one P^{L} is P.
790. The oligonucleotide of any one of Embodiments 780-789, wherein each R^{CA} is independently

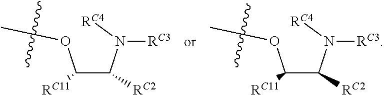

791. The oligonucleotide of any one of Embodiments 780-789, wherein each R^{CA} is independently

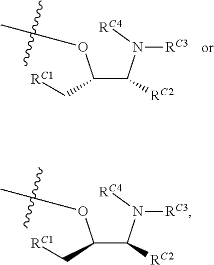

wherein R^{C1} is R, —Si(R)₃ or —SO₂R, R^{C2} and R^{C3} are taken together with their intervening atoms to form an optionally substituted 3-7 membered saturated or partially unsaturated ring having, in addition to the nitrogen atom, 0-2 heteroatoms, R^{C4} is —H or —C(O)R′.
792. The oligonucleotide of Embodiment 790 or 791, wherein in a linkage, R^{C4} is —C(O)R and P^{L} is P.
793. The oligonucleotide of any one of Embodiments 791-792, wherein in a linkage, R^{C4} is —C(O)R and W is S.
794. The oligonucleotide of any one of Embodiments 791-793, wherein in each linkage wherein W is S, R^{C4} is —C(O)R′.
795. The oligonucleotide of any one of Embodiments 791-794, wherein R^{C4} is —C(O)CH₃.
796. The oligonucleotide of Embodiment 791, wherein in a linkage, R^{C4} is —H and P^{L} is P.
797. The oligonucleotide of any one of Embodiments 791-796, wherein R^{C2} and R^{C3} are taken together with their intervening atoms to form an optionally substituted 5-membered ring having no heteroatoms in addition to the nitrogen atom.
798. The oligonucleotide of any one of Embodiments 791-797, wherein each R^{CA} is independently

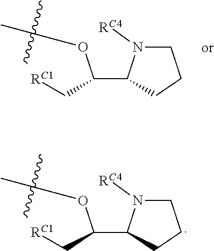

799. The oligonucleotide of any one of Embodiments 791-798, wherein R^{C1} is —SiPh₂Me.
800. The oligonucleotide of any one of Embodiments 791-798, wherein R^{C1} is —SO₂R.
801. The oligonucleotide of any one of Embodiments 791-798, wherein R^{C1} is —SO₂R, wherein R is optionally substituted C₁₋₁₀ aliphatic.
802. The oligonucleotide of any one of Embodiments 791-798, wherein R^{C1} is —SO₂R, wherein R is optionally substituted phenyl.
803. The oligonucleotide of any one of Embodiments 791-798, wherein R^{C1} is —SO₂R, wherein R is phenyl.
804. A phosphoramidite, wherein the nucleobase of the phosphoramidite is a nucleobase of any one of Embodiments 1-675 or a tautomer thereof, wherein the nucleobase or tautomer thereof is optionally substituted or protected.
805. A phosphoramidite, wherein the nucleobase is or comprises Ring BA, wherein Ring BA has the structure of BA-I, BA-I-a, BA-I-b, BA-II, BA-II-a, BA-II-b, BA-III, BA-III-a, BA-III-b, BA-IV, BA-IV-a, BA-IV-b, BA-V, BA-V-a, BA-V-b, or BA-VI, or a tautomer of Ring BA, wherein the nucleobase is optionally substituted or protected.
806. The phosphoramidite of any one of Embodiments 804-805, wherein the sugar of the phosphoramidite is a sugar of any one of Embodiments 1-675, wherein the sugar is optionally protected.
807. The phosphoramidite of any one of Embodiments 804-806, wherein the phosphoramidite has the structure of R^{NS}—P(OR)N(R)₂, wherein R^{NS} is a optionally protected nucleoside moiety, and each R is as described herein.
808. The phosphoramidite of any one of Embodiments 804-806, wherein the phosphoramidite has the structure of R^{NS}—P(OCH₂CH₂CN)N(i-Pr)₂.
809. The phosphoramidite of any one of Embodiments 804-806, wherein the phosphoramidite comprises a chiral auxiliary moiety, wherein the phosphorus is bonded to an oxygen and a nitrogen atom of the chiral auxiliary moiety.
810. The phosphoramidite of any one of Embodiments 804-806 or 809, wherein the phosphoramidite has the structure of

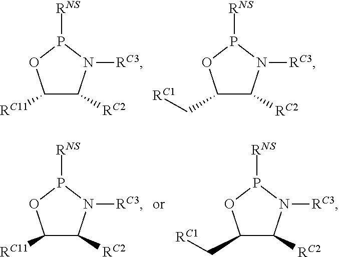

or a salt thereof.
811. The phosphoramidite of any one of Embodiments 804-806 or 809, wherein the phosphoramidite has the structure of

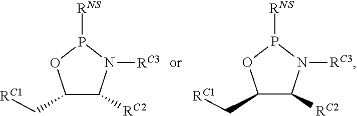

wherein R^{NS} is a optionally protected nucleoside moiety, R^{C1} is R, —Si(R)₃ or —SO₂R, R^{C2} and R^{C3} are taken together with their intervening atoms to form an optionally substituted 3-7 membered saturated or partially unsaturated ring having, in addition to the nitrogen atom, 0-2 heteroatoms.
812. The phosphoramidite of any one of Embodiments 807-811, wherein the 5′-OH of R^{NS} is protected.
813. The phosphoramidite of Embodiment 812, wherein the 5′-OH of R^{NS} is protected as —ODMTr.
814. The phosphoramidite of any one of Embodiments 807-813, wherein R^{NS} is bonded to phosphorus through its 3′-O—.
815. The phosphoramidite of any one of Embodiments 810-814, wherein R^{C2} and R^{C3} are taken together with their intervening atoms to form an optionally substituted 5-membered saturated ring having no heteroatoms in addition to the nitrogen atom.
816. The phosphoramidite of any one of Embodiments 810-815, wherein the phosphoramidite has the structure of

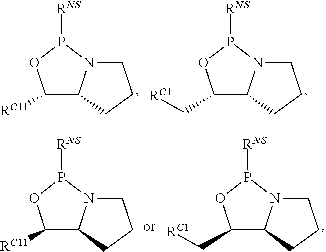

or a salt thereof.
817. The phosphoramidite of any one of Embodiments 810-815, wherein the phosphoramidite has the structure of

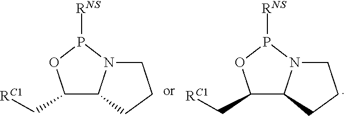

818. The phosphoramidite of any one of Embodiments 810-817, wherein R^{C1} is —SiPh₂Me.
819. The phosphoramidite of any one of Embodiments 810-817, wherein R^{C1} is —SO₂R.
820. The phosphoramidite of any one of Embodiments 810-817, wherein R^{C1} is —SO₂R, wherein R is optionally substituted C₁₋₁₀ aliphatic.
821. The phosphoramidite of any one of Embodiments 810-817, wherein R^{C1} is —SO₂R, wherein R is optionally substituted phenyl.
822. The phosphoramidite of any one of Embodiments 810-817, wherein R^{C1} is —SO₂R, wherein R is phenyl.
823. The phosphoramidites of any one of Embodiments 810-822, wherein the purity of the phosphoramidite is at least 85%, 90%, 95%, 96%, 97%, 98%, or 99%.
824. A method for preparing an oligonucleotide or composition, comprising coupling a 5′-OH of an oligonucleotide or a nucleoside with a phosphoramidite of any one of Embodiments 780-823.
825. A method for preparing an oligonucleotide or composition, comprising coupling a 5′-OH of an oligonucleotide or a nucleoside with a phosphoramidite of any one of Embodiments 810-823.
826. A method of preparing an oligonucleotide or composition, comprising removing a chiral auxiliary moiety from an oligonucleotide of any one of Embodiments 677-803.
827. The method of any one of Embodiments 824-826, wherein the oligonucleotide, or an oligonucleotide in the composition, comprises a sugar comprising 2′-OH.
828. The method of any one of Embodiments 824-827, wherein the oligonucleotide, or an oligonucleotide in the composition, comprises a sugar comprising 2′-OH, wherein the sugar is bonded to a chirally controlled internucleotidic linkage.
829. The oligonucleotide, composition or method of any one of the preceding Embodiments, wherein each heteroatom is independently selected from nitrogen, oxygen, silicon, phosphorus and sulfur.
830. The oligonucleotide, composition or method of any one of the preceding Embodiments, wherein each nucleobase independently comprises an optionally substituted ring having at least one nitrogen.
831. A method for characterizing an oligonucleotide or a composition, comprising:

administering the oligonucleotide or composition to a cell or a population thereof comprising or expressing an ADAR1 polypeptide or a characteristic portion thereof, or a polynucleotide encoding an ADAR1 polypeptide or a characteristic portion thereof 832. The method of any Embodiment 831, wherein a cell is a mouse cell.

833. The method of any one of Embodiments 831-832, wherein the genome of the cell comprises a polynucleotide encoding an ADAR1 polypeptide or a characteristic portion thereof.
834. A method for characterizing an oligonucleotide or a composition, comprising:

administering the oligonucleotide or composition to a non-human animal or a population thereof comprising or expressing an ADAR1 polypeptide or a characteristic portion thereof, or a polynucleotide encoding an ADAR1 polypeptide or a characteristic portion thereof.

835. The method of Embodiment 834, wherein the animal is a mouse.
836. The method of any one of Embodiments 834-835, wherein the genome of the animal comprises a polynucleotide encoding an ADAR1 polypeptide or a characteristic portion thereof.
837. The method of any one of Embodiments 834-835, wherein the germline genome of the animal comprises a polynucleotide encoding an ADAR1 polypeptide or a characteristic portion thereof.
838. The method of any one of Embodiments 831-837, wherein an ADAR1 polypeptide or a characteristic portion thereof is or comprises one or both of human ADAR1 Z-DNA binding domains.
839. The method of any one of Embodiments 831-838, wherein an ADAR1 polypeptide or a characteristic portion thereof is or comprises one or more or all of human ADAR1 dsRNA binding domains.
840. The method of any one of Embodiments 831-839, wherein an ADAR1 polypeptide or a characteristic portion thereof is or comprises human deaminase domain.
841. The method of any one of Embodiments 831-840, wherein an ADAR1 polypeptide or a characteristic portion thereof is or comprises human ADAR1.
842. The method of any one of Embodiments 831-841, wherein an ADAR1 polypeptide or a characteristic portion thereof is or comprises human ADAR1 p110.
843. The method of any one of Embodiments 831-841, wherein an ADAR1 polypeptide or a characteristic portion thereof is or comprises human ADAR1 p150.
844. The method of any one of Embodiments 831-843, wherein activity levels of an oligonucleotide or composition observed from a cell or a cell from an animal, or a population thereof, is more similar to those observed in a comparable human cell or a population thereof compared to those observed in a cell prior to engineering or a cell from an animal prior to engineering, or a population thereof.
845. The method of Embodiment 844, wherein a comparable human cell is of the same type as a cell or a cell from an animal.
846. A method for modifying a target adenosine in a target nucleic acid, comprising contacting the target nucleic acid with an oligonucleotide or composition of any one of the preceding Embodiments.
847. A method for deaminating a target adenosine in a target nucleic acid, comprising contacting the target nucleic acid with an oligonucleotide or composition of any one of the preceding Embodiments.
848. A method for producing, or restoring or increasing level of a product of a particular nucleic acid, comprising contacting a target nucleic acid with an oligonucleotide or composition of any one of the preceding Embodiments, wherein the target nucleic acid comprises a target adenosine, and the particular nucleic acid differs from the target nucleic acid in that the particular nucleic acid has an I or G instead of the target adenosine.
849. A method for reducing level of a product of a target nucleic acid, comprising contacting a target nucleic acid with an oligonucleotide or composition of any one of the preceding Embodiments, wherein the target nucleic acid comprises a target adenosine.
850. The method of Embodiment 848 or 849, wherein the product is a protein.
851. The method of Embodiment 848 or 849, wherein the product is a mRNA.
852. The method of any one of Embodiments 846-851, wherein the base sequence the oligonucleotide or oligonucleotides in the oligonucleotide composition is substantially complementary to that of the target nucleic acid.
853. The method of any one of Embodiments 846-852, wherein the target nucleic acid is in a sample.
854. A method, comprising:

contacting an oligonucleotide or composition of any one of the preceding Embodiments with a sample comprising a target nucleic acid and an adenosine deaminase, wherein:

the base sequence of the oligonucleotide or oligonucleotides in the oligonucleotide composition is substantially complementary to that of the target nucleic acid; and

the target nucleic acid comprises a target adenosine;

wherein the target adenosine is modified.

855. A method, comprising

1) obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and

2) obtaining a reference level of modification of a target adenosine in a target nucleic acid, which level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;

wherein:

oligonucleotides of the first plurality comprise more sugars with 2′-F modification, more sugars with 2′-OR modification wherein R is not —H, and/or more chiral internucleotidic linkages than oligonucleotides of the reference plurality; and

the first oligonucleotide composition provides a higher level of modification compared to oligonucleotides of the reference oligonucleotide composition.

856. A method, comprising

obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and

wherein the first level of modification of a target adenosine is higher than a reference level of modification of the target adenosine, wherein the reference level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;

wherein:

oligonucleotides of the first plurality comprise more sugars with 2′-F modification, more sugars with 2′-OR modification wherein R is not —H, and/or more chiral internucleotidic linkages than oligonucleotides of the reference plurality.

857. A method, comprising

1) obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and

2) obtaining a reference level of modification of a target adenosine in a target nucleic acid, which level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;

wherein:

oligonucleotides of the first plurality comprise more sugars with 2′-F modification, more sugars with 2′-OR modification wherein R is not —H, and/or more chirally controlled chiral internucleotidic linkages than oligonucleotides of the reference plurality; and

the first oligonucleotide composition provides a higher level of modification compared to oligonucleotides of the reference oligonucleotide composition.

858. A method, comprising

obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and

wherein the first level of modification of a target adenosine is higher than a reference level of modification of the target adenosine, wherein the reference level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;

wherein:

oligonucleotides of the first plurality comprise more sugars with 2′-F modification, more sugars with 2′-OR modification wherein R is not —H, and/or more chirally controlled chiral internucleotidic linkages than oligonucleotides of the reference plurality.

859. A method, comprising

1) obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and

2) obtaining a reference level of modification of a target adenosine in a target nucleic acid, which level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;

wherein:

oligonucleotides of the first plurality comprise one or more chirally controlled chiral internucleotidic linkages; and

oligonucleotides of the reference plurality comprise no chirally controlled chiral internucleotidic linkages (a reference oligonucleotide composition is a “stereorandom composition); and

the first oligonucleotide composition provides a higher level of modification compared to oligonucleotides of the reference oligonucleotide composition.

860. A method, comprising

obtaining a first level of modification of a target adenosine in a target nucleic acid, which level is observed when a first oligonucleotide composition is contacted with a sample comprising the target nucleic acid and an adenosine deaminase, wherein the first oligonucleotide composition comprises a first plurality of oligonucleotides sharing the same base sequence which is substantially complementary to that of the target nucleic acid; and

wherein the first level of modification of a target adenosine is higher than a reference level of modification of the target adenosine, wherein the reference level is observed when a reference oligonucleotide composition is contacted with a sample comprising the target nucleic acid and an adenosine deaminase, wherein the reference oligonucleotide composition comprises a reference plurality of oligonucleotides sharing the same base sequence which is substantially complementary to that of the target nucleic acid;

wherein:

oligonucleotides of the first plurality comprise one or more chirally controlled chiral internucleotidic linkages; and

oligonucleotides of the reference plurality comprise no chirally controlled chiral internucleotidic linkages (a reference oligonucleotide composition is a “stereorandom composition).

861. The method of any one of Embodiments 855-860, wherein a first oligonucleotide composition is an oligonucleotide composition of any one of the preceding Embodiments.
862. The method of any one of Embodiments 855-861, wherein the reference oligonucleotide composition is a reference oligonucleotide composition of any one of Embodiments 767-776.
863. The method of any one of Embodiments 846-862, wherein the deaminase is an ADAR enzyme.
864. The method of any one of Embodiments 846-862, wherein the deaminase is ADAR1.
865. The method of any one of Embodiments 846-862, wherein the deaminase is ADAR2.
866. The method of any one of Embodiments 846-865, wherein the target nucleic acid is or comprise RNA.
867. The method of any one of Embodiments 846-866, wherein a sample is a cell.
868. The method of any one of Embodiments 846-867, wherein the target nucleic acid is more associated with a condition, disorder or disease, or decrease of a desired property or function, or increase of an undesired property or function, compared to a nucleic acid which differs from the target nucleic acid in that it has an I or G at the position of the target adenosine instead of the target adenosine.
869. The method of any one of Embodiments 846-867, wherein the target adenosine is a G to A mutation.
870. A method for preventing or treating a condition, disorder or disease amenable to a G to A mutation, comprising administering to a subject susceptible thereto or suffering therefrom an effective amount of an oligonucleotide or composition of any one of the preceding Embodiments.
871. A method for preventing or treating a condition, disorder or disease associated with a G to A mutation, comprising administering to a subject susceptible thereto or suffering therefrom an effective amount of an oligonucleotide or composition of any one of the preceding Embodiments.
872. The method of Embodiment 870 or 871, wherein the base sequence of the oligonucleotide or oligonucleotides in the oligonucleotide composition is substantially complementary to that of the target nucleic acid comprising a target adenosine that is the mutation.
873. The method of any one of Embodiments 871-872, wherein the condition, disorder or disease is amenable to an A to G or A to I modification.
874. The method of any one of Embodiments 870-873, wherein cells associated with the condition, disorder or disease comprise or express an ADAR protein.
875. The method of any one of Embodiments 870-873, wherein cells associated with the condition, disorder or disease comprise or express ADAR1.
876. The method of any one of Embodiments 870-873, wherein cells associated with the condition, disorder or disease comprise or express ADAR2.
877. The method of any one of Embodiments 870-876, wherein the condition, disorder or disease is or is associated with Alpha-1 antitrypsin deficiency.
878. The method of any one of Embodiments 846-877, comprising converting a target adenosine to I.
879. The method of any one of Embodiments 846-878, wherein two or more different adenosine are targeted and edited.
880. The method of any one of Embodiments 846-878, wherein two or more different transcripts are targeted and edited.
881. The method of any one of Embodiments 846-878, wherein transcripts from two or more different polynucleotides are targeted and edited.
882. The method of any one of Embodiments 846-878, wherein transcripts from two or more genes are targeted and edited.
883. The method of any one of Embodiments 879-882, comprising administering two or more oligonucleotides, each of which independently targets a different target, and each of which is independently an oligonucleotide of any one of Embodiments 1-675 or a salt thereof.
884. The method of any one of Embodiments 879-882, comprising administering two or more oligonucleotide compositions, each of which independently targets at least one different target, and each of which is independently a composition of any one of Embodiments 676-779.
885. The method of any one of Embodiments 879-884, comprising administering a composition of any one of Embodiments 701-779.
886. The method of any one of Embodiments 879-885, wherein two or more oligonucleotides or compositions are administered concurrently.
887. The method of any one of Embodiments 879-886, wherein two or more oligonucleotides or compositions are administered concurrently in a single composition.
888. The method of any one of Embodiments 879-886, wherein two or more oligonucleotides or compositions are administered as separated compositions.
889. The method of any one of Embodiments 879-885, wherein one or more oligonucleotides or compositions are administered prior or subsequently to one or more other oligonucleotides or compositions.

### EXEMPLIFICATION

Certain examples of provided technologies (compounds (oligonucleotides, reagents, etc.), compositions, methods (methods of preparation, use, assessment, etc.), etc.) were presented herein.

Those skilled in the art appreciates that many technologies can be utilized to assess properties and/or activities of provided technologies, e.g., those described in Examples below.

### Example 1. Useful Technologies for Assessing Adenosine Editing

Oligonucleotide designs may be assessed using various systems. In some embodiments, cLuc oligonucleotides were prepared and assessed in HEK293T cells. In some embodiments, oligonucleotides targeting cLuc (Cypridina) were assessed in 293T cells transfected with plasmids for either human ADAR1 or human ADAR2 and a cLuc luciferase reporter plasmid. The cLuc reporter plasmid consisted of (Gaussia)gLuc-p2A-cLuc(W85X) with respect to luciferases. The cLuc reporter was activated by ADAR mediated A>I editing. The editing activity of oligonucleotides was calculated using the equation: Fold change=oligonucleotides treated (cLuc/gLuc)/mock (cLuc/gLuc)

In some embodiments, reporter plasmid and ADAR1 or ADAR2 plasmid were transfected together into HEK293T cells using the Liptofectamine 2000 transfection protocol (Thermo 11668030). After a suitable time period, e.g., 24 hours, the HEK293T cells expressing the reporter and ADAR plasmids were reverse transfected with the appropriate amount of oligonucleotides for each experiment. cLuc and gLuc activity was measured after 48, 72, and/or 96 hours using the Pierce™ Gaussia Luciferase Glow Assay Kit (Pierce™ 16161) or the Pierce™ Cypridina Luciferase Glow Assay Kit (Pierce™ 16170), respectively.

### Example 2. Provided Technologies Provide Desired Editing without Exogenous ADAR

In some embodiments, oligonucleotides and compositions were assessed in primary human hepatocytes to assess adenosine editing. Oligonucleotides targeting ACTB mRNA were transfected using either the Liptofectamine RNAimax transfection protocol (Thermo 11668030) or delivered under Gymnotic free uptake conditions into primary human hepatocytes (Gibco). After 48 hours, total RNA was collected using the SV 96 Total RNA Isolation System Protocol (Promega: Z3505). Primers flanking the edit site of the endogenously expressed target RNA were for PCR using the Phusion High-Fidelity DNA Polymerase protocol (Thermo: F-530XL). The PCR product was Sanger-sequenced and the percent of ADAR mediated editing was calculated using the program EditR (https://moriaritylab.shinyappsio/editr_v10/). Provided technologies provided desired editing without exogenous ADAR.

### Example 3. Provided Technologies Provide Desired Editing without Exogenous ADAR in Various Cell Types

In some embodiments, oligonucleotides and compositions thereof were assessed in primary bronchial epithelial cells to assess adenosine editing. Oligonucleotides targeting ACTB mRNA were transfected using either the Liptofectamine RNAimax transfection protocol (Thermo 11668030) or delivered under Gymnotic free uptake conditions into primary bronchial epithelial cells (Lonza). After 48 hours, total RNA was collected using the SV 96 Total RNA Isolation System Protocol (Promega: Z3505). Primers flanking the edit site of the endogenously expressed target RNA were for PCR using the Phusion High-Fidelity DNA Polymerase protocol (Thermo: F-530XL). The PCR product was Sanger-sequenced and the percent of ADAR mediated editing was calculated using the program EditR. Provided technologies provided editing without exogenous ADAR.

### Example 4. Provided Technologies Provide Desired Editing without Exogenous ADAR in Various Cell Types

In some embodiments, oligonucleotides and compositions were assessed in primary retinal pigment epithelial cells for adenosine editing. Oligonucleotides targeting ACTB mRNA were transfected using either the Liptofectamine RNAimax transfection protocol (Thermo 11668030) or delivered under Gymnotic free uptake conditions into primary human retinal pigment epithelial cells (Lonza). After 48 hours, total RNA was collected using the SV 96 Total RNA Isolation System Protocol (Promega: Z3505). Primers flanking the edit site of the endogenously expressed target RNA were used for PCR using the Phusion High-Fidelity DNA Polymerase protocol (Thermo: F-530XL). The PCR product was Sanger-sequenced and the percent of ADAR mediated editing was calculated using the program EditR. Provided technologies provided editing without exogenous ADAR.

### Example 5. Provided Technologies Provide Desired Editing of Various Target Nucleic Acids

In some embodiments, oligonucleotides and compositions were assessed in primary mouse hepatocyte cells. Primary hepatocytes were isolated from the NOD.Cg-Prkdcscid Il2rgtmlWjl Tg(SERPINA1*E342K) #Slcw/SzJ mouse model (The Jackson Laboratory: 028842). Oligonucleotides targeting SerpinA1 mRNA were transfected using either the Liptofectamine RNAimax transfection protocol (Thermo 11668030) or delivered under Gymnotic free uptake conditions into the isolated primary hepatocytes. After 48 hours, total RNA was collected using the SV 96 Total RNA Isolation System Protocol (Promega: Z3505). Primers flanking the edit site of the endogenously expressed target RNA were used for PCR using the Phusion High-Fidelity DNA Polymerase protocol (Thermo: F-530XL). The PCR product was Sanger-sequenced and the percent of ADAR mediated editing was calculated using the program EditR. Provided technologies provided desired editing.

### Example 6. Provided Technologies can Provide Editing Activity

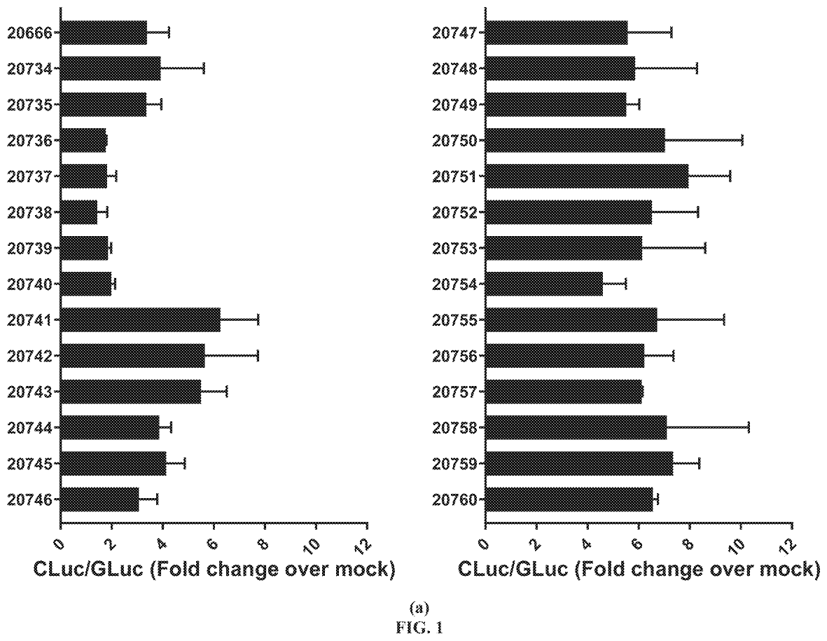

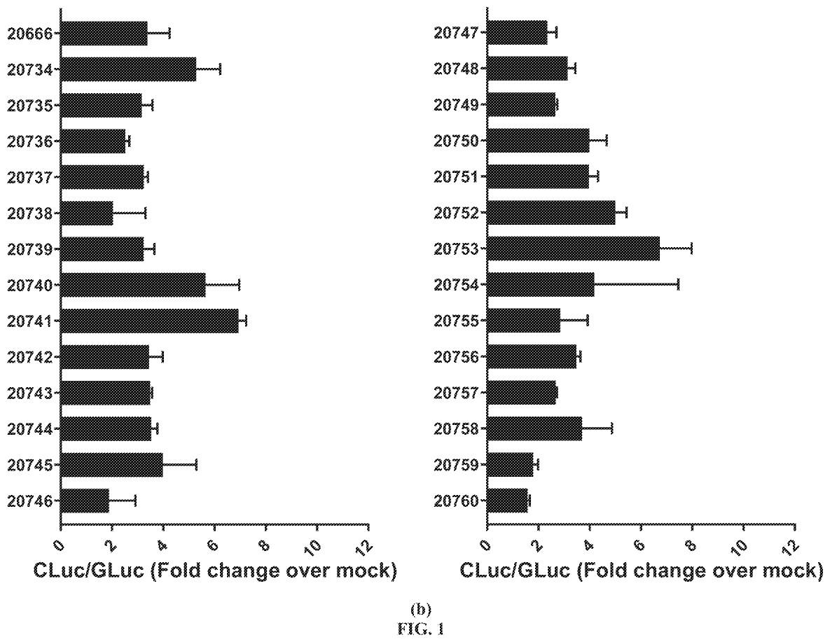

In some embodiments, the present disclosure provides oligonucleotide compositions that can, among other things, deliver various activities, e.g., adenosine editing activities. For example, compositions of oligonucleotides comprising provided sugar modification designs can provide editing activities. Certain data were presented in, e.g., FIG. 1 and several other Figures. As demonstrated, in some embodiments one or more sugar modifications at certain positions (e.g., 2′-F in first domains, 2′-OMe in second domains, etc.) optionally with other modifications (e.g., modified internucleotidic linkages such as phosphorothioate internucleotidic linkages) may improve editing levels in accordance with the present disclosure.

### Example 7. Provided Technologies can Provide Editing Activity

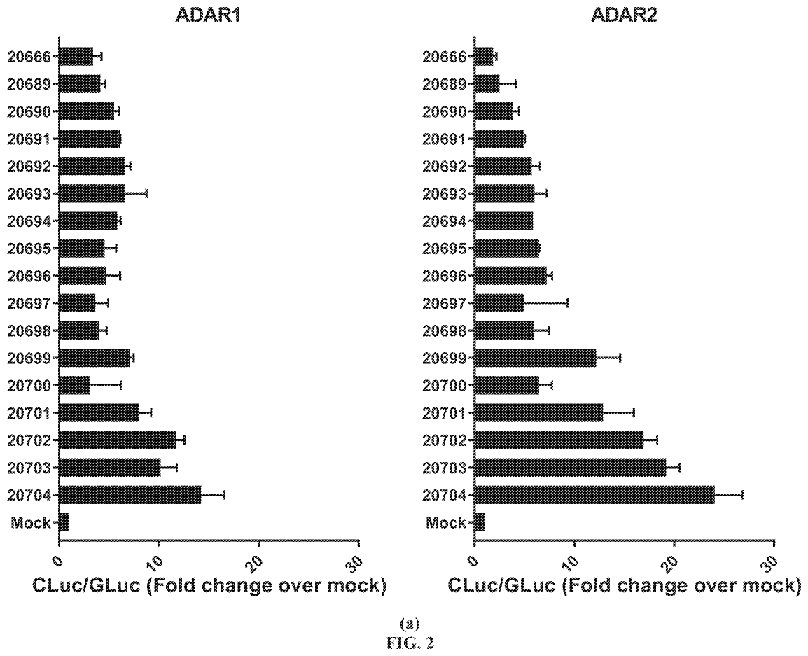

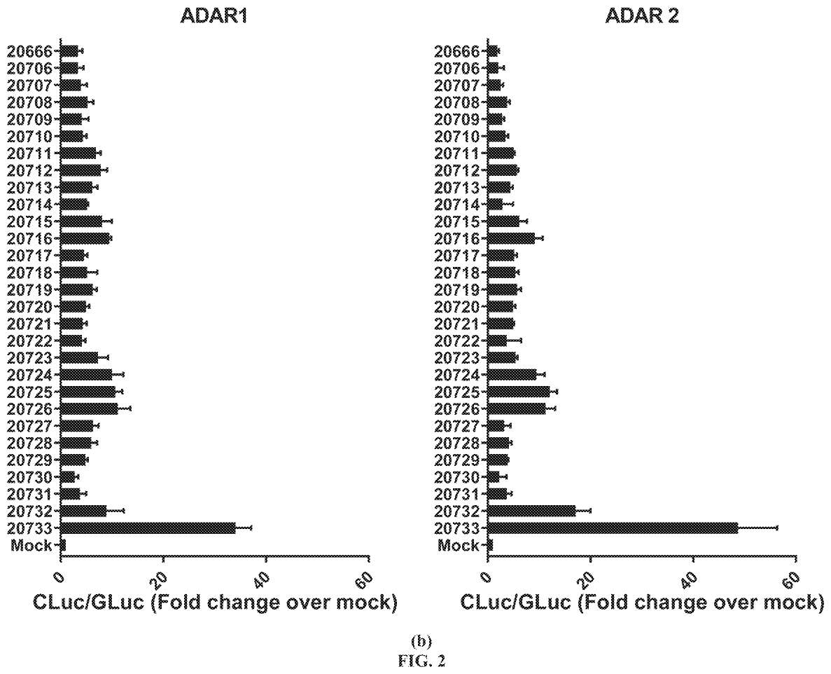

In some embodiments, the present disclosure provides oligonucleotide compositions that can, among other things, deliver various activities, e.g., adenosine editing activities. For example, compositions of oligonucleotides comprising provided internucleotidic linkage modification designs can provide editing activities. Certain data were presented in, e.g., FIG. 2 and several other Figures. As shown in FIG. 2, in some embodiments incorporation of one or more modified internucleotidic linkages, e.g., phosphorothioate internucleotidic linkages, may improve editing levels in accordance with the present disclosure.

### Example 8. Provided Technologies Comprising Various Sugar Modifications can Provide Editing Activities

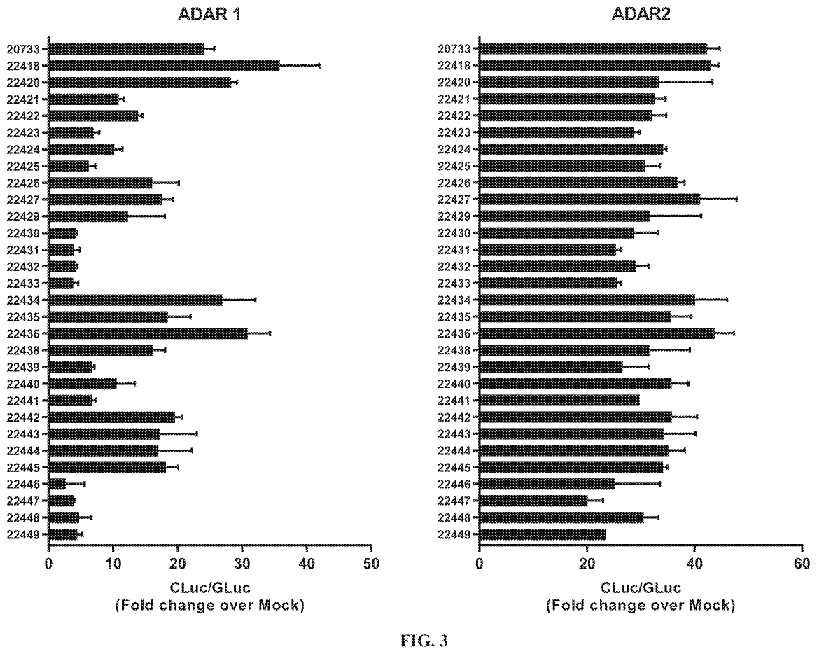

Among other things, the present disclosure demonstrates that oligonucleotides comprising various sugar modifications and compositions thereof can provide editing activities as shown, e.g., in various Figures. Certain data are presented in FIG. 3, in which various oligonucleotide compositions were assessed. 293T cells were transfected with ADAR1 or ADAR2, luciferase reporter construct and indicated compositions. cLuc activity was measure and normalized to Gluc expression in mock treated samples (n=2 biological replicates). As demonstrated, in some embodiments utilization of certain sugars, e.g., natural DNA sugars, 2′-F modified sugars, etc. at and/or near editing sites provide editing activities. In some embodiments, an oligonucleotide comprises 5′-N₁N₀N₋₁-3′, wherein each of N₁, N₀, and N₋₁ is independently a nucleoside, N1 and N₀ bond to an internucleotidic linkage as described herein, and N₋₁ and N₀ bond to an internucleotidic linkage as described herein, and N₀ is opposite to a target adenosine. In some embodiments, the sugar of each of N₁, N₀, and N₋₁ is independently a natural DNA sugar. In some embodiments, the sugar of N₁ is a 2′-modified sugar, and the sugar of each of N₀ and N₋₁ is independently a natural DNA sugar. In some embodiments, such oligonucleotides provide high editing levels.

### Example 9. Provided Technologies Comprising Various Sugar Types can Provide Editing Activities

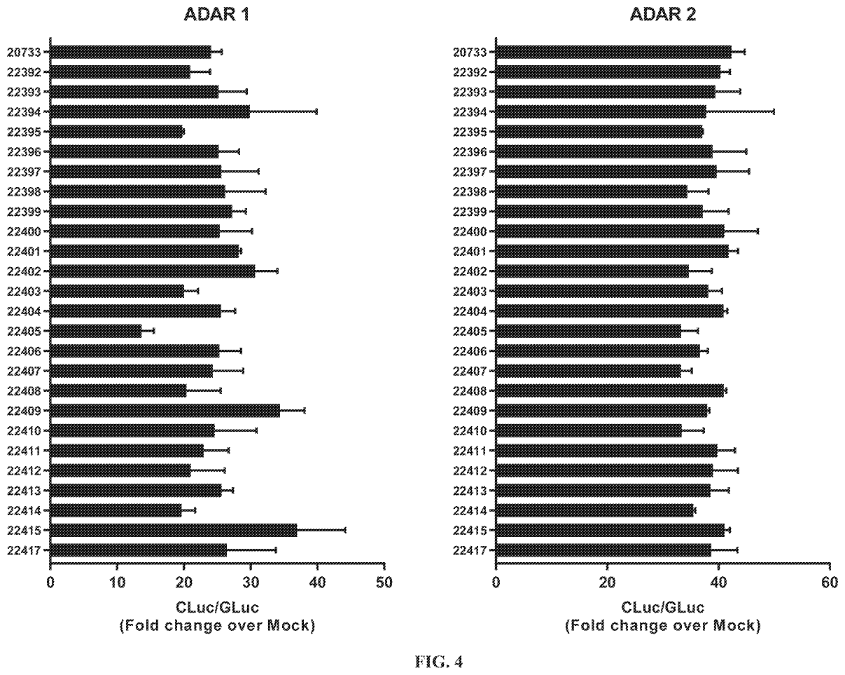

Among other things, the present disclosure demonstrates that oligonucleotides comprising various sugar types and compositions thereof can provide editing activities as shown, e.g., in various Figures. Certain data are presented in FIG. 4, in which various oligonucleotide compositions were assessed. 293T cells were transfected with ADAR1 or ADAR2, luciferase reporter construct and indicated compositions. cLuc activity was measure and normalized to Gluc expression in mock treated samples (n=2 biological replicates).

### Example 10. Provided Technologies can Provide Editing Activities with Short Sequences

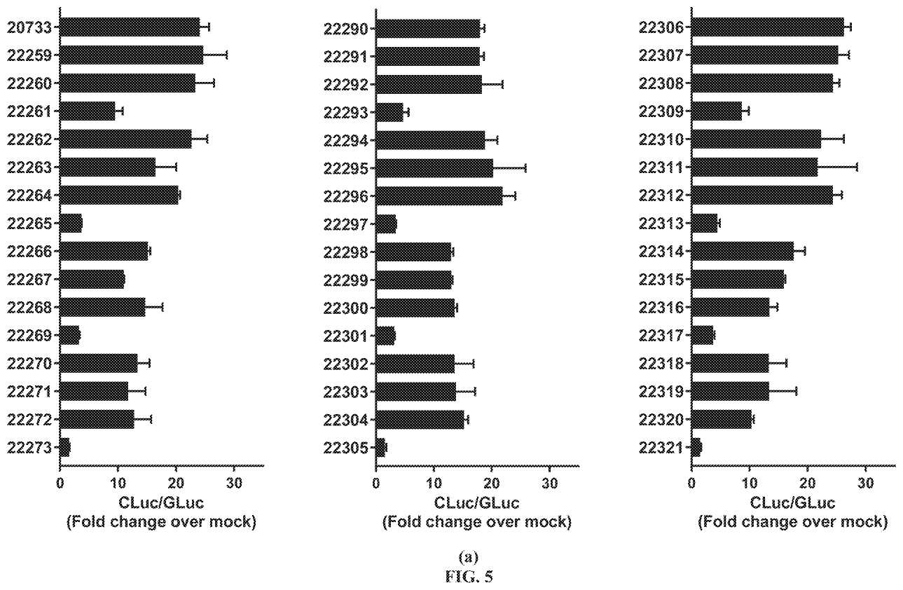

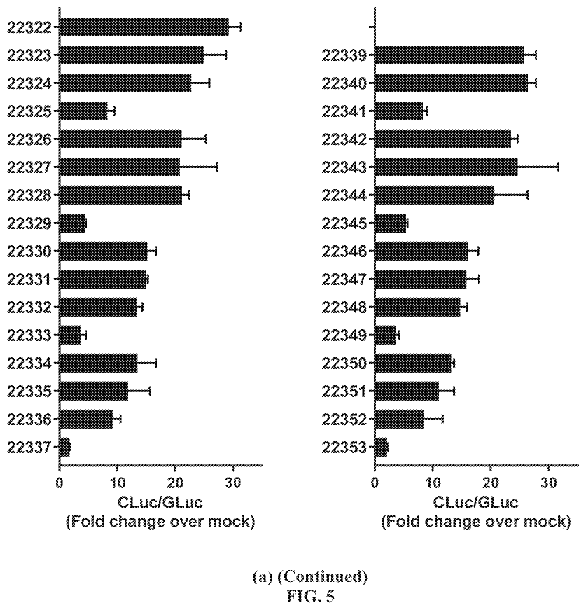

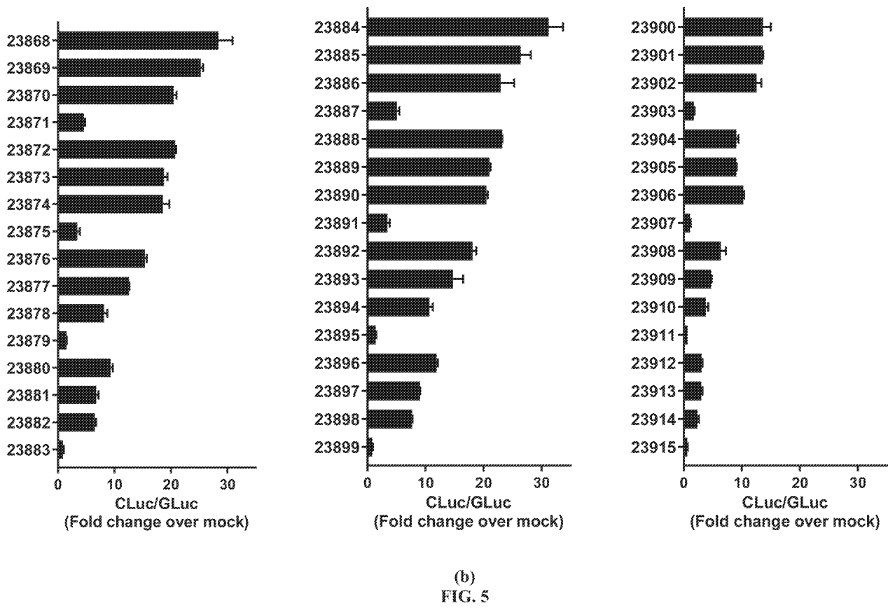

In some embodiments, the present disclosure provides oligonucleotides of short sequences compared to, e.g., prior technologies, and compositions thereof. Such oligonucleotides and compositions can provide a number of advantages, e.g., for manufacturing, delivery, etc. As demonstrated in, e.g., various Figures, provided oligonucleotides and compositions can provide editing activities without using long sequences. Certain data are presented in FIG. 5, in which various oligonucleotide compositions were assessed. 293T cells were transfected with ADAR1 (a and b) or ADAR2 (c and d), luciferase reporter construct and indicated compositions. cLuc activity was measured and normalized to Gluc expression in mock treated samples (n=2 biological replicates). As demonstrated, oligonucleotides of various lengths, e.g., 27, 28, 29, 20, 31, 32, or more, can provide editing, e.g., when contacted with ADAR1 and/or ADAR2.

### Example 11. Provided Technologies can Provide Editing Activities in Various Cell Types without Exogenous ADAR

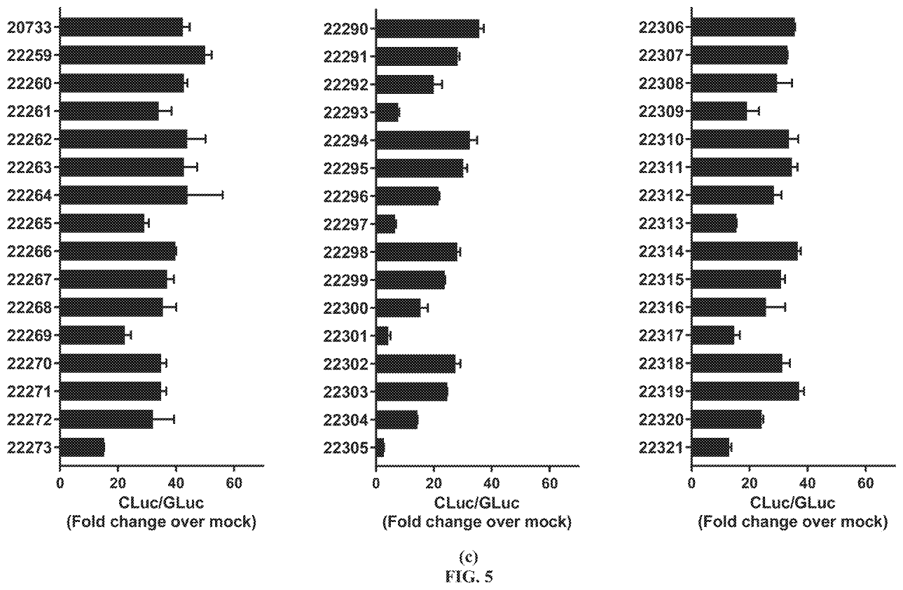

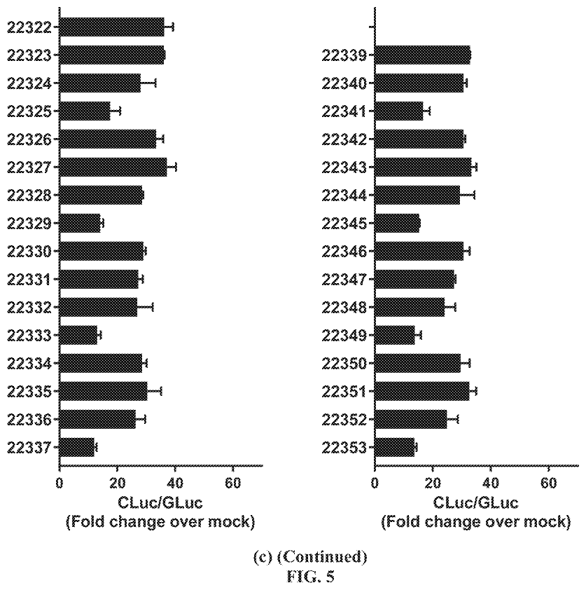

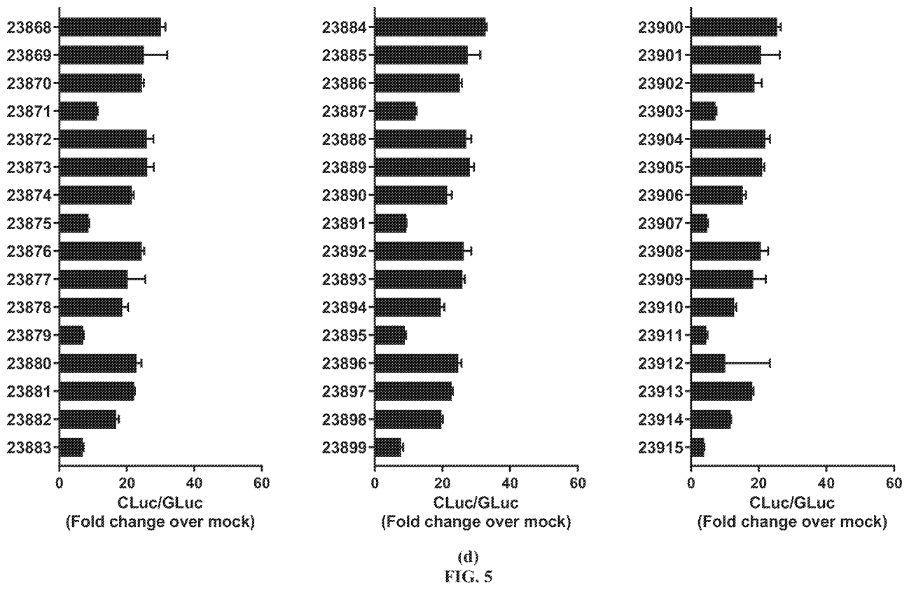

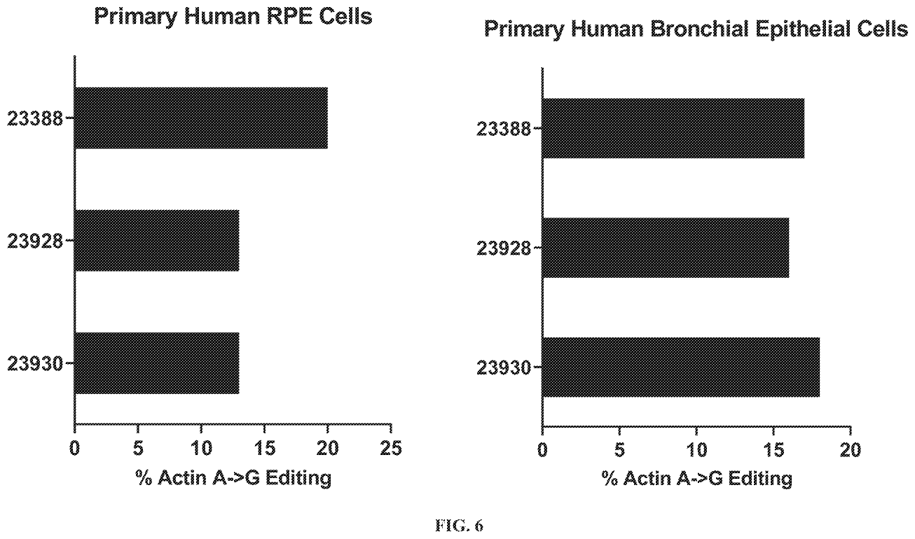

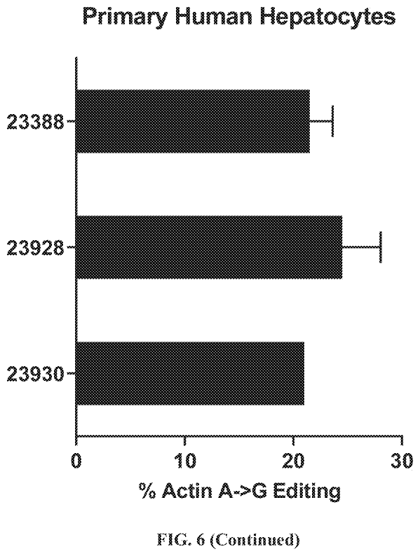

In some embodiments, provided technologies do not require exogenous ADAR to provide desired results, e.g., as shown in various Figures. Certain data are presented in FIG. 6, which depicts editing of an endogenous target (TAG site in 3′ UTR of actin) without exogenous ADAR in different cell types. Cells were transfected with 50 nM oligonucleotides and editing was measured 48 hours later. (N=1 for RPE and NHBE cells, N=2 biological replicates for Hepatocytes)

### Example 12. Provided Technologies Comprising Various Numbers of Mismatches can Provide Editing Activities

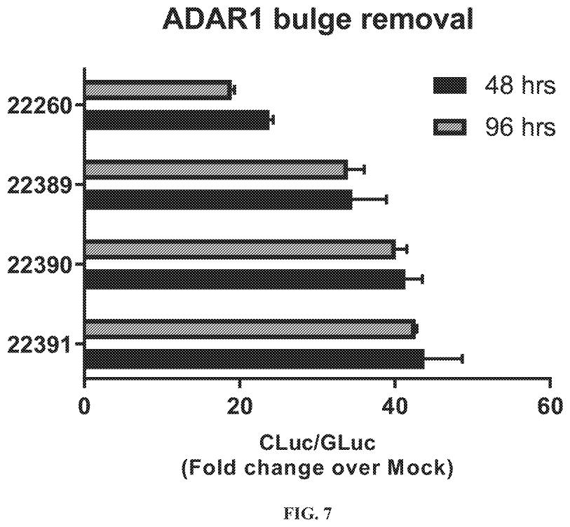

In some embodiments, provided oligonucleotides are fully complementary to target nucleic acids. In some embodiments, there are one or more mismatches. Among other things, the present disclosure demonstrates that oligonucleotides comprising various numbers of mismatches and compositions thereof can provide editing activities, e.g. as shown in various Figures. Certain data are presented in FIG. 7, in which various oligonucleotide compositions were assessed. 293T cells were transfected with ADAR1 or ADAR2, luciferase reporter construct and indicated compositions. cLuc activity was measured at 48 and 96 hrs and normalized to Gluc expression in mock treated samples (n=2 biological replicates).

### Example 13. Provided Technologies Comprising Various Patterns of Mismatches can Provide Editing Activities

In some embodiments, mismatches have various distances in between and are at various locations (e.g., relative to ends, nucleosides opposite to target adenosine, etc.). Among other things, the present disclosure demonstrates that various patterns of mismatches can be utilized to provide editing activities, e.g. as shown in various Figures. Certain data are presented in FIG. 8, in which various oligonucleotide compositions were assessed. 293T cells were transfected with ADAR1 or ADAR2, luciferase reporter construct and indicated compositions. cLuc activity was normalized to Gluc expression in mock treated samples (n=2 biological replicates).

### Example 14. Provided Technologies Comprising Various Patterns of Mismatches can Provide Editing Activities

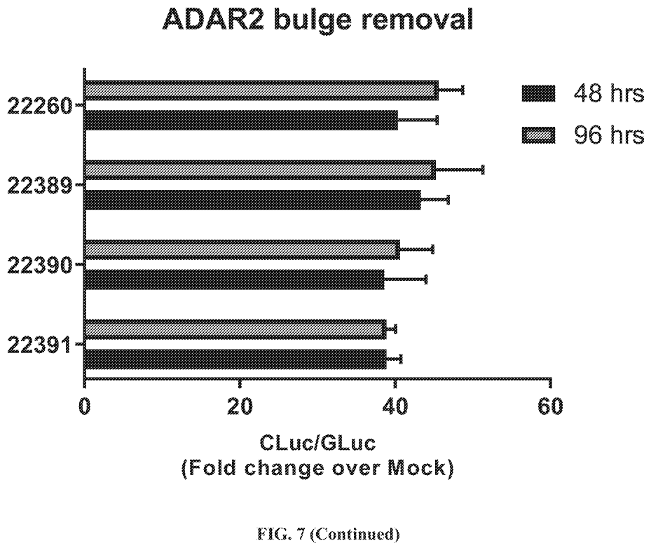

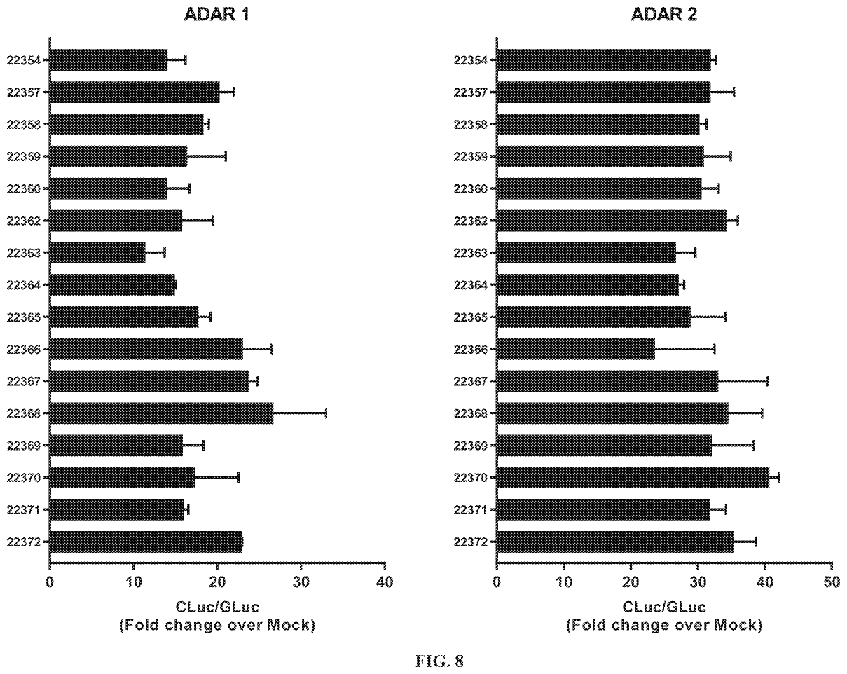

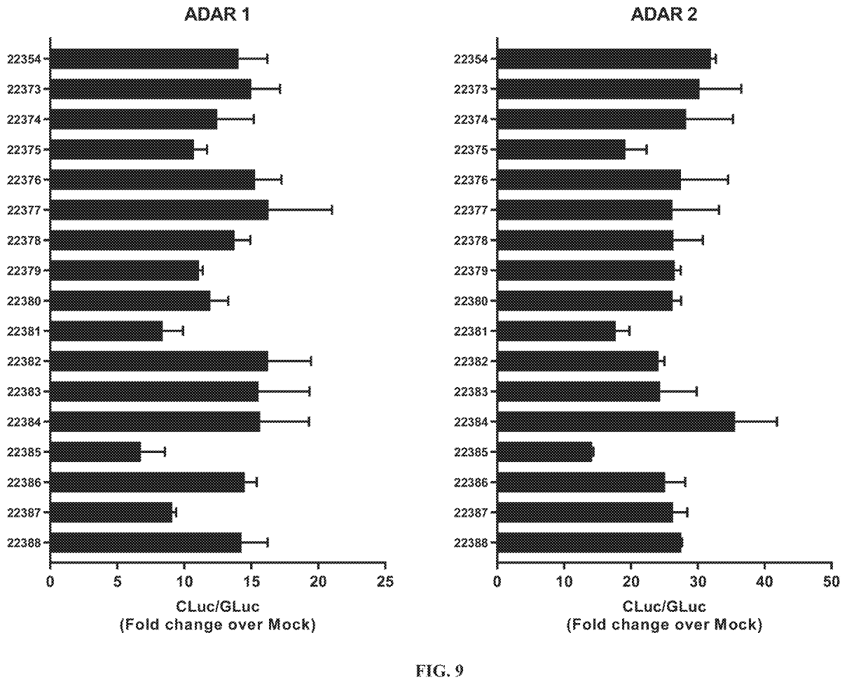

FIG. 9 provides additional data showing that oligonucleotides having various mismatch patterns and compositions thereof can provide editing activities. For FIG. 9, compositions all target a premature UAG stop codon within the cLuc coding sequence. 293T cells were transfected with ADAR1 or ADAR2, luciferase reporter construct and indicated compositions. cLuc activity was normalized to Gluc expression in mock treated samples (n=2 biological replicates).

### Example 15. Chirally Controlled Oligonucleotide Compositions can Provide High Activities

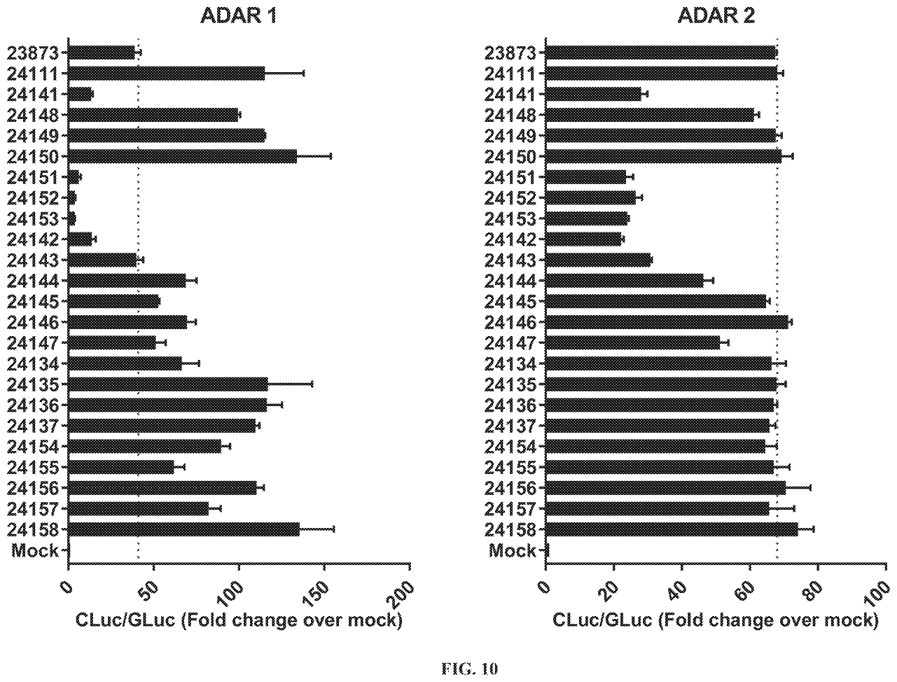

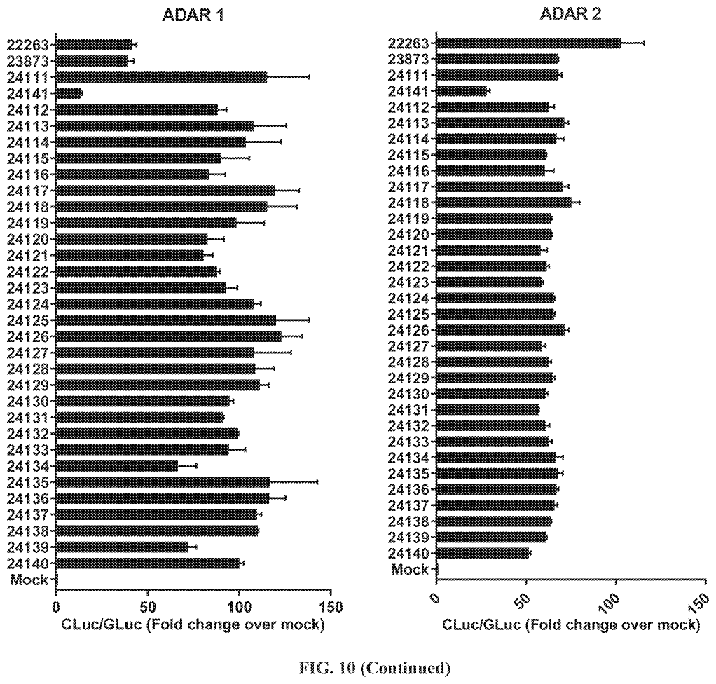

Among other things, the present disclosure provides chirally controlled oligonucleotide compositions which can provide various advantages over corresponding stereorandom compositions. In some embodiments, as shown, e.g., in various Figures, chirally controlled oligonucleotide compositions can provide editing activities. Without the intention to be bound by any particularly theory, chirally controlled internucleotidic linkages may provide improved oligonucleotide stability and/or facilitate adenosine editing by ADAR proteins through certain stereochemically specific interactions with ADAR. Certain data are presented in FIG. 10, in which various oligonucleotide compositions were assessed. 293T cells were transfected with ADAR1 or ADAR2, luciferase reporter construct and indicated compositions. cLuc activity was normalized to Gluc expression in mock treated samples (n=2 biological replicates). Among other things, it was demonstrated that chirally controlled oligonucleotide compositions of oligonucleotides comprising Sp internucleotidic linkages, e.g., Sp phosphorothioate internucleotidic linkages, can provide higher levels of editing (e.g., compared to reference stereorandom compositions and/or certain chirally controlled oligonucleotide compositions of oligonucleotides comprising fewer Sp and/or more Rp phosphorothioate internucleotidic linkages). In some embodiments, Rp phosphorothioate internucleotidic linkages may be utilized in positions in the middle of oligonucleotides in accordance with the present disclosure, e.g., bonded to nucleosides of second subdomains, etc.

### Example 16. Chirally Controlled Oligonucleotide Compositions can Provide High Activities

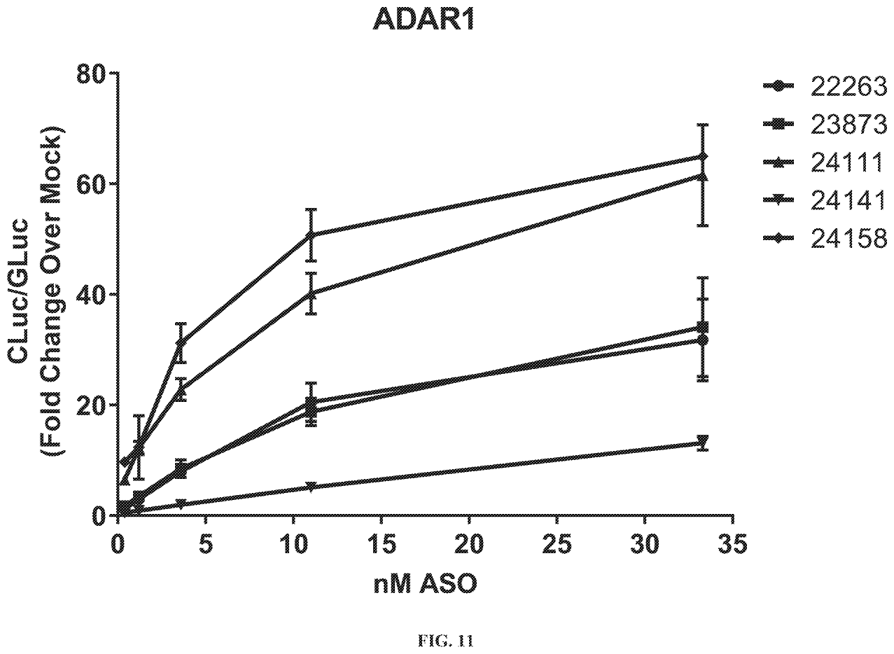

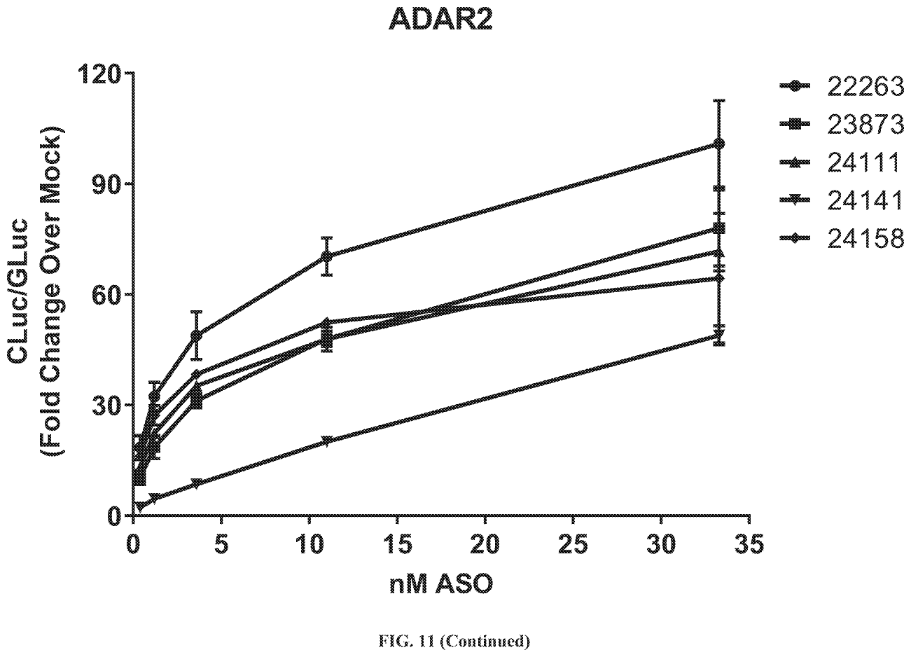

In some embodiments, the present disclosure provides chirally controlled oligonucleotide compositions that can, among other things, deliver high activities, e.g., adenosine editing activities. In some embodiments, as shown in FIG. 11, chirally controlled oligonucleotide compositions can deliver up to 8 to 10 fold higher ADAR1-mediated editing activities compared to a reference stereorandom oligonucleotide composition.

### Example 17. Chirally Controlled Oligonucleotide Compositions can Provide Significantly Higher Activities in Various Cell Types without Exogenous ADAR

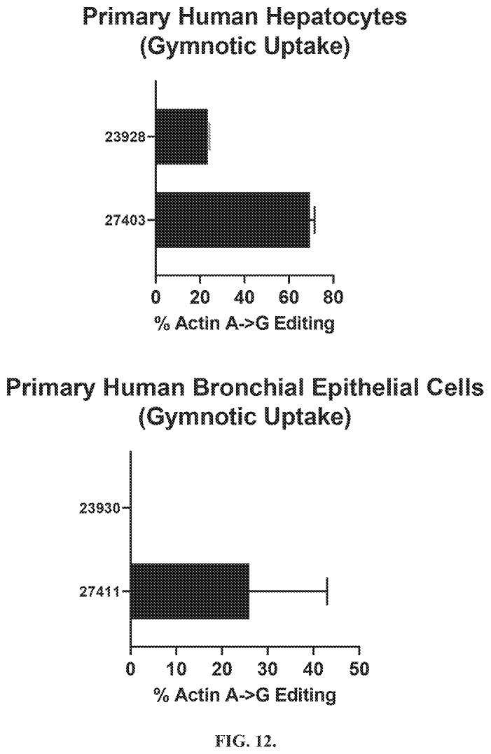

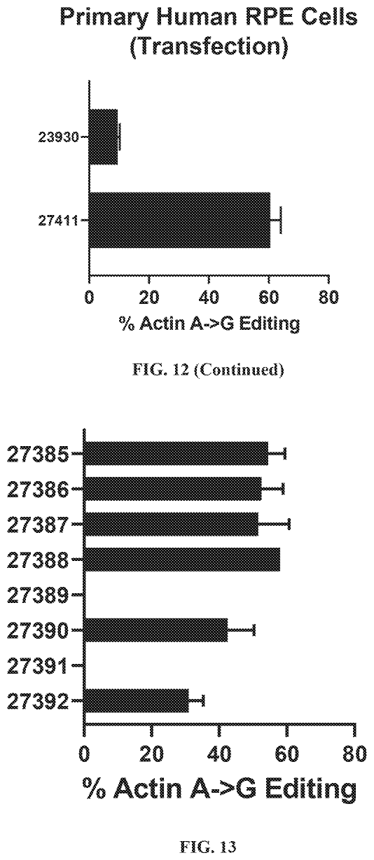

Among other things, the present disclosure demonstrates that chirally controlled oligonucleotide compositions can provided unexpected high activities without exogenous ADAR. Certain data are presented in FIG. 12, in which various oligonucleotide compositions were assessed. Cells were treated gymnotically with oligonucleotides at 10 uM dose, or transfected at 50 nM dose. RNA was harvested 48 hours later and percentage of edited transcripts was quantified by Sanger sequencing (n=2 biological replicates).

### Example 18. Provided Technologies can Provide Editing Activities with Short Sequences without Exogenous ADAR

As demonstrated herein, provided technologies can deliver editing activities using oligonucleotides of various length and compositions thereof. Certain data are presented in FIG. 13, depicts editing in primary human retinal pigmented epithelial (RPE cells). Compositions all target a UAG motif in the 3′UTR of actin. Primary human RPE cells were transfected with 50 nM of oligonucleotides. RNA was harvested 48 hours later and percentage of edited transcripts was quantified by Sanger sequencing (n=2 biological replicates).

### Example 19. Chirally Controlled Oligonucleotide Compositions can Provide High Activities in Various Cell Types without Exogenous ADAR

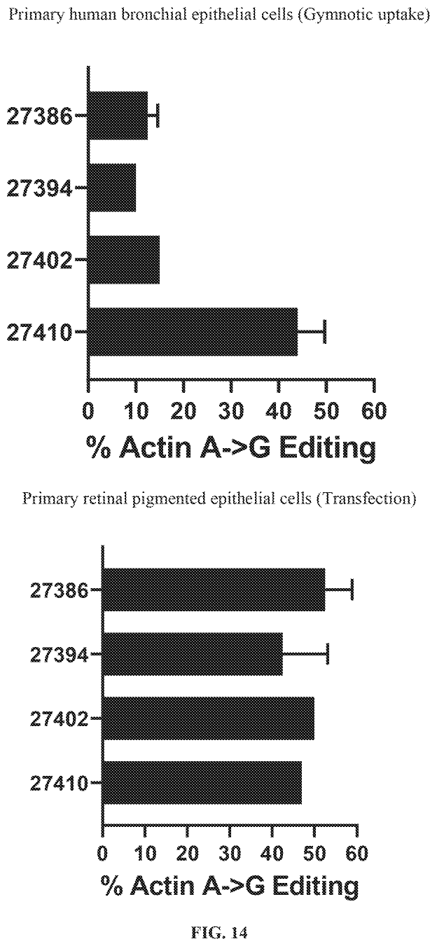

Certain advantages of provided technologies, e.g., high activities are demonstrated in FIG. 14, in which various oligonucleotide compositions were assessed. Primary human bronchial epithelial cells were treated gymnotically with 10 uM of oligonucleotides, while primary RPE cells were transfected with 50 nm of oligonucleotides. RNA was harvested 48 hours later and percentage of edited transcripts was quantified by Sanger sequencing (n=2 biological replicates).

### Example 20. Provided Technologies Comprising Various Internucleotidic Linkage Patterns can Provide Editing Activities

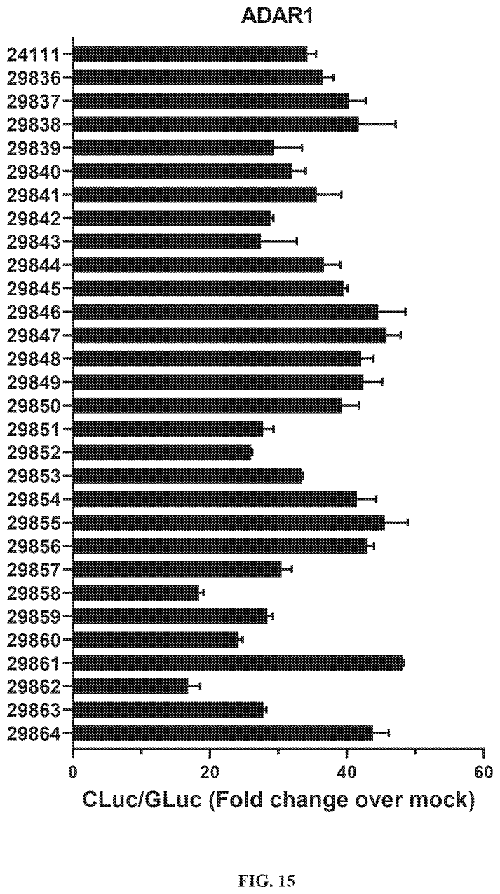

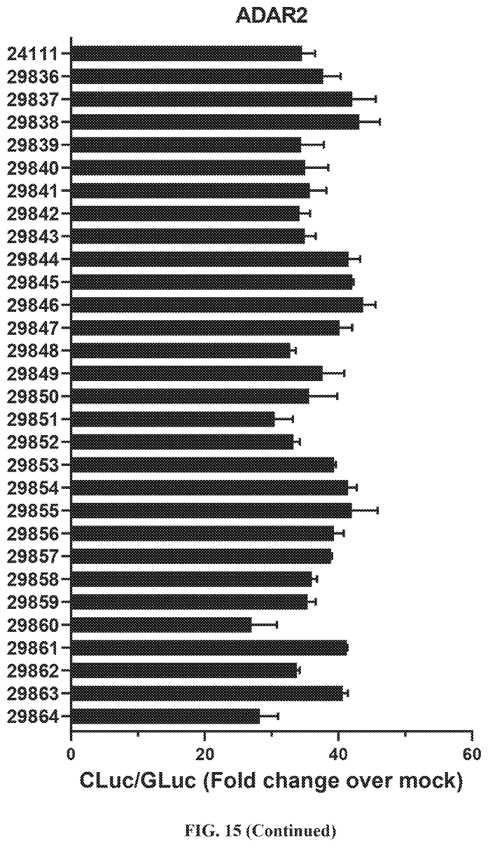

Among other things, the present disclosure provides oligonucleotides having various types of internucleotidic linkages (and patterns thereof). Certain internucleotidic linkages, e.g., neutral internucleotidic linkages, may improve one or more properties and/or activities of oligonucleotides. Among other things, the present disclosure demonstrates that various types of internucleotidic linkages can be utilized to provide editing editing activity, e.g., as shown in various Figures. Certain data are presented in FIG. 15, in which various oligonucleotide compositions were assessed. 293T cells were transfected with ADAR1 or ADAR2, luciferase reporter construct and indicated compositions. cLuc activity was normalized to Gluc expression in mock treated samples (n=2 biological replicates).

### Example 21. Provided Technologies Comprising Various Internucleotidic Linkage Patterns can Provide Editing Activities without Exogenous ADAR

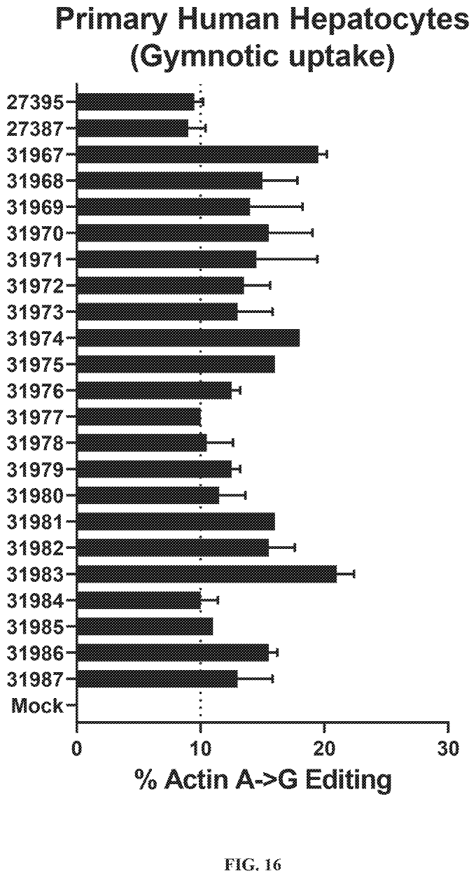

Among other things, the present disclosure demonstrates that provided technologies comprising various internucleotidic linkage patterns can provide editing activities without exogenous ADAR, for example, as illustrated in FIG. 16, in which various oligonucleotide compositions were assessed. Primary human hepatocytes were treated gymnotically with 3.3 uM oligonucleotides. RNA was harvested 48 hours later and percentage of edited transcripts was quantified by Sanger sequencing (n=2 biological replicates).

### Example 22. Provided Technologies Comprising Various Modifications and Chiral Control can Provide Editing Activities without Exogenous ADAR

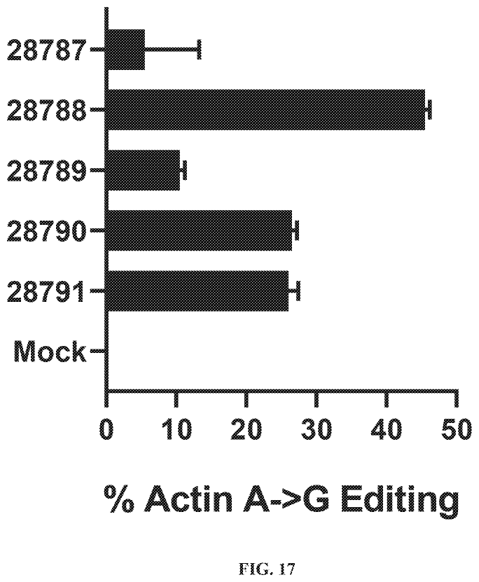

As described herein, provided technologies can utilize various provided features, e.g., modifications and/or stereochemistry control, to provide oligonucleotides and compositions of high activities. In some embodiments, provided modifications, e.g., at nucleosides opposite to target adenosine, can provide high adenosine editing activities without exogenous ADAR, e.g., as shown in FIG. 17, in which various oligonucleotide compositions were assessed. Primary human hepatocytes were transfected with 50 nM of oligonucleotides. RNA was harvested 48 hours later and percentage of edited transcripts was quantified by Sanger sequencing (n=2 biological replicates).

### Example 23. Provided Technologies Comprising Additional Moieties can Provide Editing Activities without Exogenous ADAR

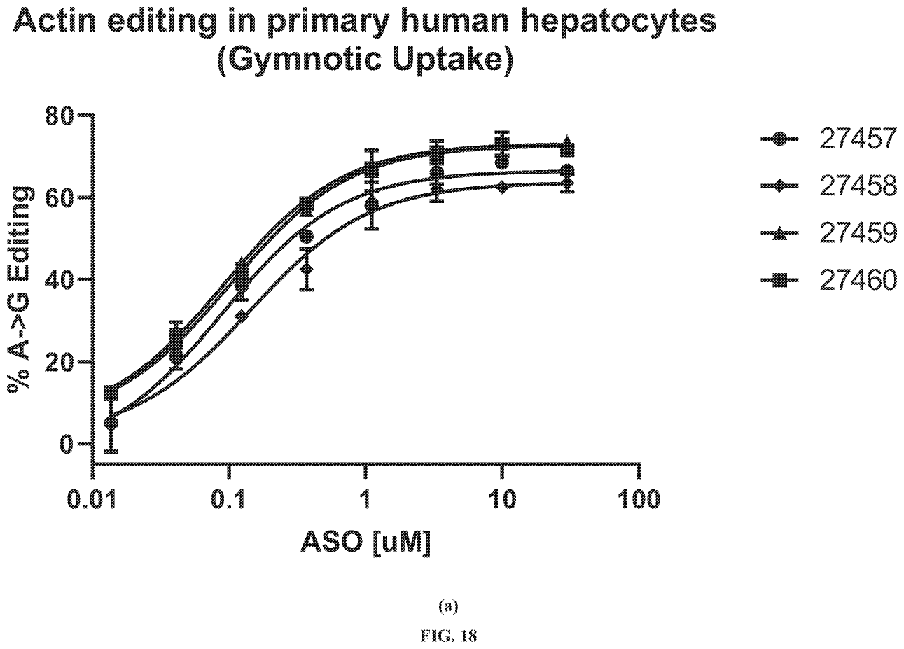

In some embodiments, provided oligonucleotides comprise additional chemical moieties, e.g., GalNAc and derivatives thereof. In some embodiments, incorporation of additional moieties provide improved properties and/or activities. Certain data were presented in FIG. 18, in which compositions of oligonucleotides comprising GalNAc moieties were assessed. Primary human hepatocytes were gymnotically treated at varying concentrations. Editing of target was measured by Sanger sequencing (n=2 biological replicates). In one set of data in primary human hepatocytes, editing by chirally controlled oligonucleotide compositions WV-27458 and WV-27460 reached 65%-75% of ACTB, and absolute EC50 for WV-27458 and WV-27460 were 309.0 nM (95% CI: 259.0-359.1 nM) and 166.2 nM (95% CI: 144.6-187.8 nM), respectively, whereas editing by stereorandom composition WV-30298 plateaued with less than 50%, confirming that chirally controlled oligonucleotide compositions can provide, among other things, improved editing. As described and demonstrated herein, chiral control can provide, among other things, increased activities when various delivery technologies are utilized. For example, in one set of experiments wherein gymnotic delivery were utilized (bronchial epithelial cells, 10 uM oligonucleotide concentration), no ACTB editing was observed for stereorandom composition WV-23928, while chirally controlled oligonucleotide composition WV-27387 provided about 19% editing of ACTB.

### Example 24. Provided Technologies Comprising Modified Nucleobases can Provide Editing Activities without Exogenous ADAR

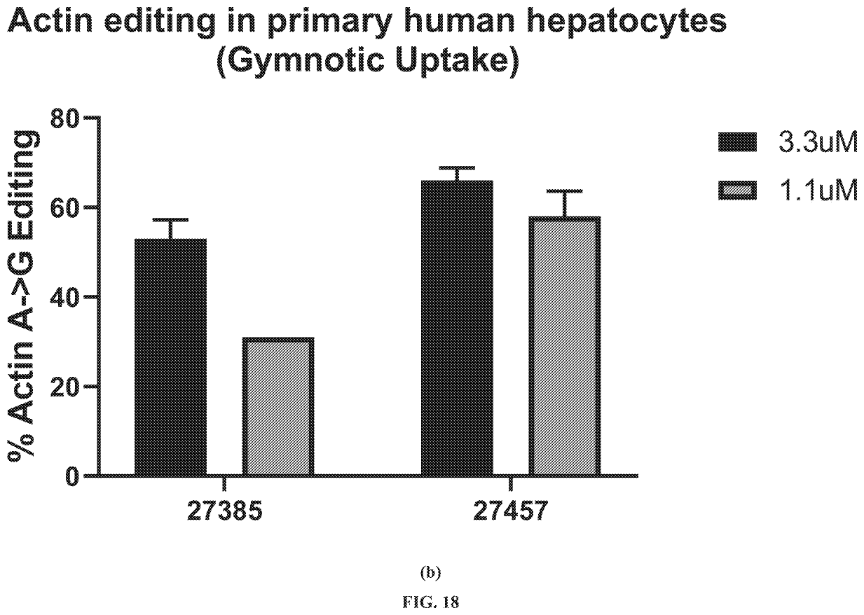

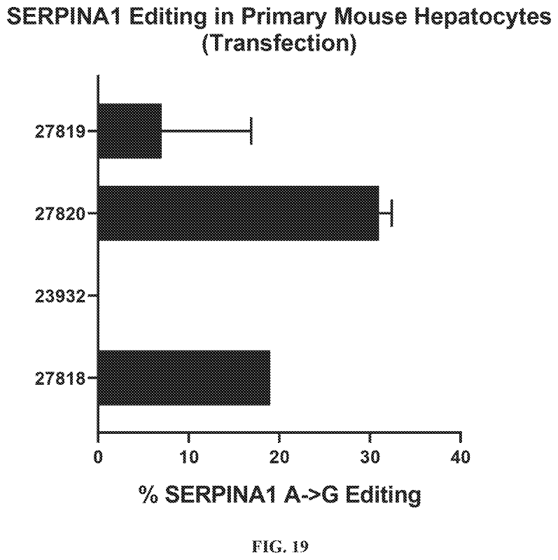

In some embodiments, provided technologies comprise modified nucleobases at certain locations, and such modified nucleobases may provide various advantages, e.g., high activities. In some embodiments, as demonstrated herein, e.g., in FIG. 19, oligonucleotides comprising modified nucleobases as designed can provide significantly higher activities (e.g., hypoxanthine next to a nucleobase opposite to target adenine). FIG. 19 depicts editing of SERPINA1 (PiZ allele) in primary mouse hepatocytes. Compositions all target an adenosine in the mutant human SERPINA1 transcript (PiZZ allele). Primary hepatocytes (extracted from a mouse model expressing the mutant human transcript) were transfected with 50 nM of oligonucleotides. Editing of target was measured by Sanger sequencing (n=2 biological replicates).

### Example 25. Provided Technologies Comprising Modified Nucleobases can Provide Editing Activities without Exogenous ADAR

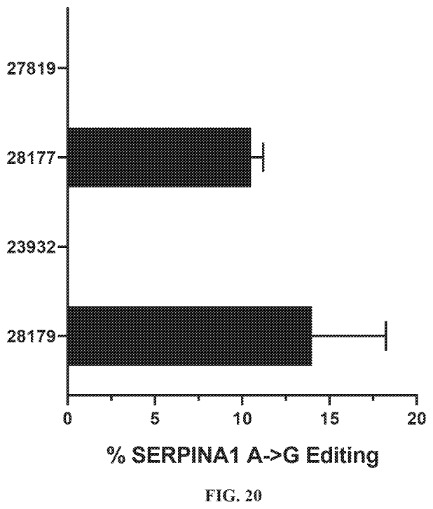

In some embodiments, provided technologies comprise modified nucleobases at certain locations, e.g., opposite to target adenosine, and such modified nucleobases may provide various advantages, e.g., high activities. In some embodiments, as demonstrated herein, e.g., in FIG. 20, oligonucleotides comprising modified nucleobases (in FIG. 20, pseudo-isocytosine) as designed can provide significantly higher activities. FIG. 20 depicts editing of SERPINA1 (PiZ allele) in primary mouse hepatocytes. Compositions all target an adenosine in the mutant human SERPINA1 transcript (PiZZ allele). Primary hepatocytes (extracted from a mouse model expressing the mutant human transcript) were transfected treated with 50 nM oligonucleotides. Editing of target was measured by Sanger sequencing (n=2 biological replicates). As shown, provided designs comprising modified nucleobases can greatly improve activities.

### Example 26. Provided Technologies Comprising Various Nucleobases can Provide Editing Activity

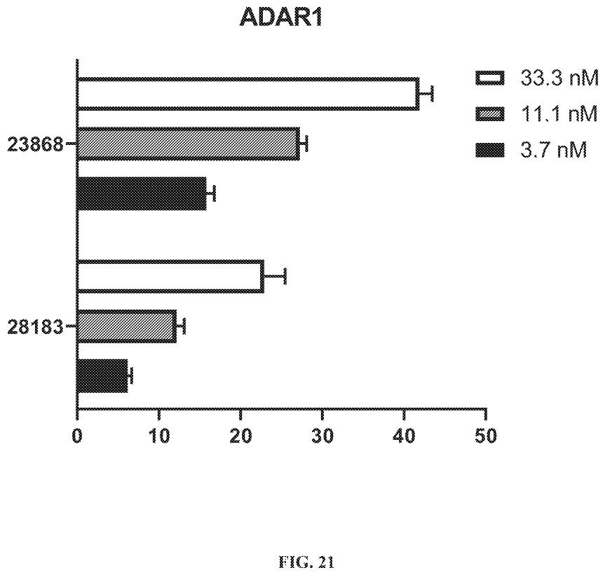

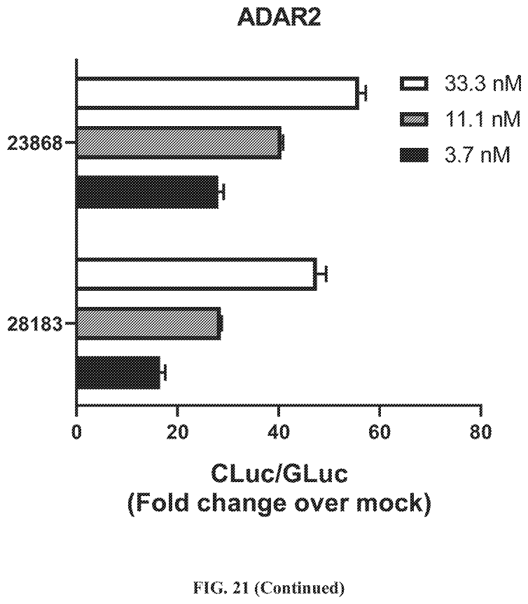

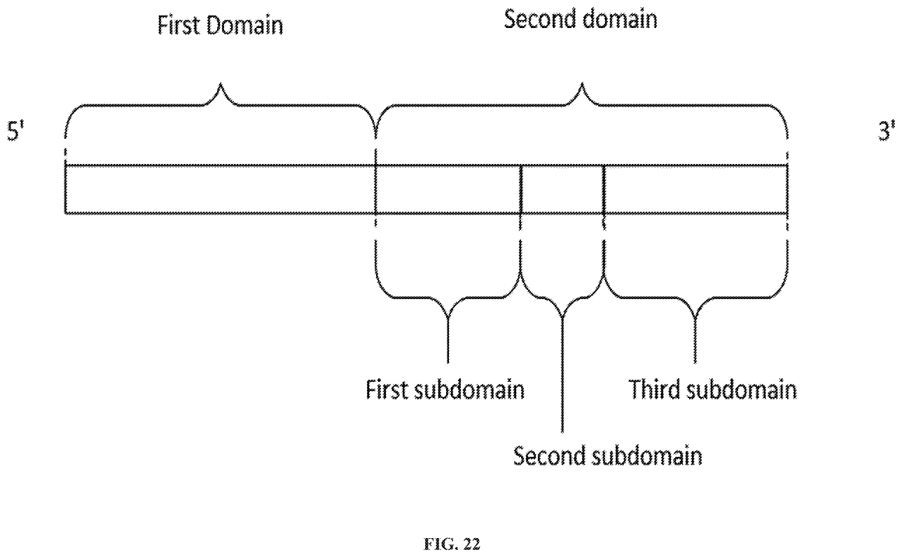

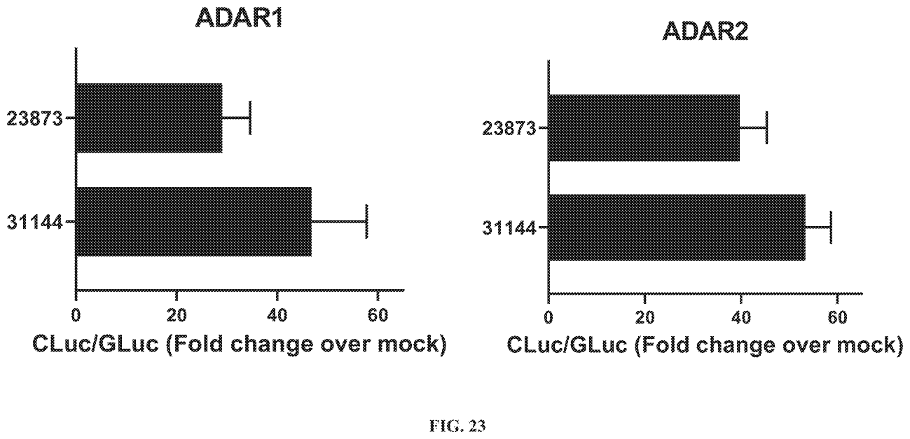

Among other things, the present disclosure demonstrates that oligonucleotides comprising various nucleobases, including modified nucleobases, may provide editing. Certain data are presented in FIG. 21. 293T cells were transfected with ADAR1 or ADAR2, luciferase reporter construct and indicated compositions at varying oligonucleotide concentrations. cLuc activity was normalized to Gluc expression in mock treated samples (n=2 biological replicates).

### Example 27. Provided Technologies Comprising Various Nucleobases can Provide Editing Activity

Among other things, the present disclosure demonstrates that oligonucleotides comprising various nucleobases, including modified nucleobases, may provide enhanced editing. In some embodiments, nucleosides comprising modified nucleobases are directly across from target adenosine (in some embodiments, such nucleosides are referred to as “nucleosides opposite to target adenosine”, or “opposite nucleosides”). Certain data are presented in FIG. 21, in which compositions of oligonucleotides comprising 8-oxoadenosine were assessed. 293T cells were transfected with ADAR1 or ADAR2, luciferase reporter construct and indicated compositions at varying oligonucleotide concentrations. cLuc activity was normalized to Gluc expression in mock treated samples (n=2 biological replicates). As demonstrated herein, 8-oxoadenosine can provide higher editing efficiency. Without the intention to be limited by any theory, 8-oxoadenosine could form more and/or stronger hydrogen bond(s) than, e.g., cytosine, with an Adar enzyme as it has an N—H bond at the 7′ position of its purine ring.

### Example 28. Synthesis of 1-((2R,3R,4R,5R)-5-((bis(4-methoxyphenyl)(phenyl)methoxy)methyl)-3-((tert-butyldimethylsilyl)oxy)-4-hydroxytetrahydrofuran-2-yl)-3,4-dihydropyrimidin-2(1H)-one

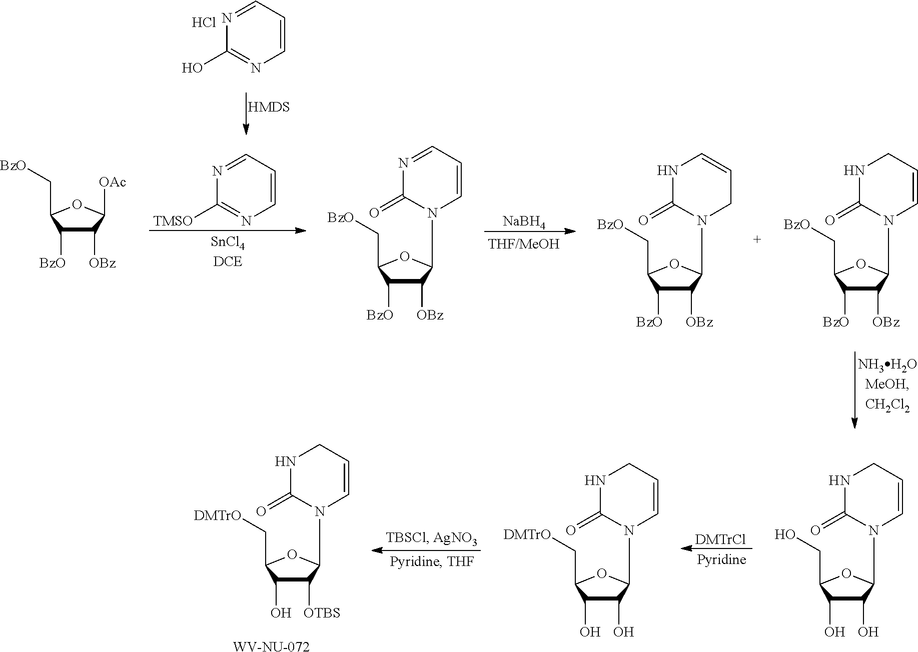

Step 1. A mixture of pyrimidin-2-ol HCl salt (40 g, 301.78 mmol, 1 eq.) in HMDS (800 mL) was degassed and purged with N₂ for 3 times, and then the mixture was stirred at 130° C. for 2 hr under N₂ atmosphere. The reaction mixture becomes clear. The reaction mixture was added toluene (500 mL *2) then concentrated under reduced pressure to remove solvent. The crude product 2-((trimethylsilyl)oxy)pyrimidine (50 g, crude, as yellow oil) was used into the next step without further purification.

Step 2. To a solution of (2S,3R,4R,5R)-2-acetoxy-5-((benzoyloxy)methyl)tetrahydrofuran-3,4-diyl dibenzoate (60 g, 118.93 mmol, 1 eq.) in DCE (1200 mL) was added compound 2-((trimethylsilyl)oxy)pyrimidine (26.82 g, 159.37 mmol, 1.34 eq.) SnCl₄ (43.69 g, 167.70 mmol, 19.59 mL, 1.41 eq.) in DCE (600 mL) and Molecular sieve 4A (10 g) was added. The mixture was stirred at 15° C. for 12 hr. The reaction mixture was concentrated under reduced pressure to remove solvent. The residue was purified by column chromatography (SiO₂, Petroleum ether/Ethyl acetate=1/1 to 0:1) to obtained (2R,3R,4R,5R)-2-((benzoyloxy)methyl)-5-(2-oxopyrimidin-1(2H)-yl)tetrahydrofuran-3,4-diyl dibenzoate (128 g, 99.56% yield) was obtained as a yellow solid. TLC: (Petroleum ether:Ethyl acetate=0:1): R_{f}=0.32. MS: 445.2 (M-95)⁺.

Step 3. To a solution of (2R,3R,4R,5R)-2-((benzoyloxy)methyl)-5-(2-oxopyrimidin-1(2H)-yl)tetrahydrofuran-3,4-diyl dibenzoate (40 g, 74.00 mmol, 1 eq.) in THF (2000 mL) was added NaBH4 (3.08 g, 81.40 mmol, 1.1 eq.) in MeOH (100 mL). The mixture was stirred at 0° C. for 1 hr. The reaction mixture was concentrated under reduced pressure to remove solvent. The residue was added H₂O (1500 mL) then diluted with EtOAc (2000 mL) and extracted with EtOAc (2000 mL *3). Dried over Na₂SO₄, filtered and concentrated under reduced pressure to give a residue. The residue was purified by column chromatography (SiO₂, Petroleum ether/Ethyl acetate=2:3) to obtain (2R,3R,4R,5R)-2-((benzoyloxy)methyl)-5-(2-oxo-3,6-dihydropyrimidin-1(2H)-yl)tetrahydrofuran-3,4-diyl dibenzoate (13 g, 23.96 mmol, 10.79% yield) was obtained as a yellow solid and (2R,3R,4R,5R)-2-((benzoyloxy)methyl)-5-(2-oxo-3,4-dihydropyrimidin-1(2H)-yl)tetrahydrofuran-3,4-diyl dibenzoate (19 g, 35.02 mmol, 15.77% yield) was obtained as a yellow solid. MS: 543.0 (M+H)⁺.

Step 4. To a solution of (2R,3R,4R,5R)-2-((benzoyloxy)methyl)-5-(2-oxo-3,4-dihydropyrimidin-1(2H)-yl)tetrahydrofuran-3,4-diyl dibenzoate (4 g, 7.37 mmol, 1 eq.) in MeOH (40 mL) and DCM (20 mL) was added NH₃·H₂O (25.84 g, 184.32 mmol, 28.40 mL, 25% purity, 25 eq). The reaction mixture was concentrated under reduced pressure to remove the solvent, then the crude was dried by azeotropic distillation on a rotary evaporator with toluene (30 mL*2). The crude was purified by reversed-phase HPLC (0.1% NH₃H₂O condition) to obtain 1-((2R,3R,4S,5R)-3,4-dihydroxy-5-(hydroxymethyl)tetrahydrofuran-2-yl)-3,4-dihydropyrimidin-2(1H)-one (1.7 g, 7.38 mmol, 100% yield) as an yellow solid. ¹H NMR (400 MHz, DMSO-d₆) δ=7.97-7.76 (m, 1H), 7.51-7.28 (m, 2H), 6.78 (br s, 1H), 6.32 (br d, J=8.1 Hz, 1H), 5.63 (br d, J=5.6 Hz, 1H), 4.95-4.80 (m, 1H), 3.90-3.82 (m, 2H), 3.77 (br d, J=12.7 Hz, 1H), 3.65 (br d, J=2.7 Hz, 1H), 3.54-3.36 (m, 2H); MS: 230.8 (M+H)⁺.

Step 5. To a solution of 1-((2R,3R,4S,5R)-3,4-dihydroxy-5-(hydroxymethyl)tetrahydrofuran-2-yl)-3,4-dihydropyrimidin-2(1H)-one (1 g, 4.34 mmol, 1 eq.) in pyridine (20 mL) was added DMTr-Cl (1.62 g, 4.77 mmol, 1.1 eq.). The mixture was stirred at 15° C. for 2 hr. The reaction mixture was concentrated under reduced pressure to remove solvent. The residue was purified by prep-HPLC (column: Agela DuraShell C18 150*25 mm*5 um; mobile phase: [water (0.04% NH₃H₂O)-ACN]; B %: 35%-55%, 22 min). 1-((2R,3R,4S,5R)-5-((bis(4-methoxyphenyl)(phenyl)methoxy)methyl)-3,4-dihydroxytetrahydrofuran-2-yl)-3,4-dihydropyrimidin-2(1H)-one (0.3 g, 563.29 umol, 12.98% yield) was obtained as a yellow solid. ¹H NMR (400 MHz, CHLOROFORM-d) 6=7.42 (d, J=7.7 Hz, 2H), 7.36-7.28 (m, 5H), 7.22 (d, J=7.3 Hz, 1H), 6.83 (d, J=8.8 Hz, 4H), 6.44 (d, J=8.2 Hz, 1H), 5.84-5.70 (m, 1H), 5.03 (br s, 1H), 4.84-4.69 (m, 1H), 4.21 (d, J=3.3 Hz, 2H), 4.10 (br s, 1H), 3.98 (br s, 2H), 3.80 (s, 6H), 3.50 (s, 1H), 3.38 (br d, J=3.1 Hz, 1H), 3.26 (dd, J=3.6, 10.5 Hz, 2H)

Step 5. To a solution of 1-((2R,3R,4S,5R)-5-((bis(4-methoxyphenyl)(phenyl)methoxy)methyl)-3,4-dihydroxytetrahydrofuran-2-yl)-3,4-dihydropyrimidin-2(1H)-one (0.7 g, 1.31 mmol, 1 eq.) in THF (32 mL) was added AgNO₃ (267.93 mg, 1.58 mmol, 1.2 eq.) and pyridine (519.82 mg, 6.57 mmol, 530.43 uL, 5 eq.) then added TBSCl (217.91 mg, 1.45 mmol, 177.16 uL, 1.1 eq.). The reaction mixture was added with sat. NaHCO₃(aq., 5 mL) and extracted with EtOAc (10 mL*3). The combined organic layers were dried over Na₂SO₄, filtered and concentrated under reduced pressure to give a residue. The residue was purified by prep-TLC (SiO₂, Petroleum ether:Ethyl acetate=1:1). 1-((2R,3R,4R,5R)-5-((bis(4-methoxyphenyl)(phenyl)methoxy)methyl)-3-((tert-butyldimethylsilyl)oxy)-4-hydroxytetrahydrofuran-2-yl)-3,4-dihydropyrimidin-2 (1H)-one (WV-NU-072) (0.39 g, 602.93 umol, 45.87% yield) was obtained as a white solid. ¹H NMR (400 MHz, CHLOROFORM-d) 6=7.42 (d, J=7.5 Hz, 2H), 7.36-7.27 (m, 6H), 7.25-7.19 (m, 1H), 6.84 (d, J=8.6 Hz, 4H), 6.46 (br d, J=8.1 Hz, 1H), 5.92 (d, J=6.0 Hz, 1H), 4.83 (br s, 1H), 4.79-4.70 (m, 1H), 4.32 (t, J=5.8 Hz, 1H), 4.13 (br d, J=7.0 Hz, 1H), 4.06-4.00 (m, 1H), 3.99-3.90 (m, 2H), 3.80 (s, 6H), 3.44 (dd, J=2.5, 10.5 Hz, 1H), 3.26 (dd, J=3.1, 10.4 Hz, 1H), 1.00-0.89 (m, 9H), 0.18 (d, J=2.6 Hz, 6H).

### Example 29. Synthesis of -benzyl-1-((2R,3R,4R,5R)-5-((bis(4-methoxyphenyl)(phenyl)methoxy)methyl)-3-((tert-butyldimethylsilyl)oxy)-4-hydroxytetrahydrofuran-2-yl)-2,4-dioxo-1,2,3,4-tetrahydropyrimidine-5-carboxamide

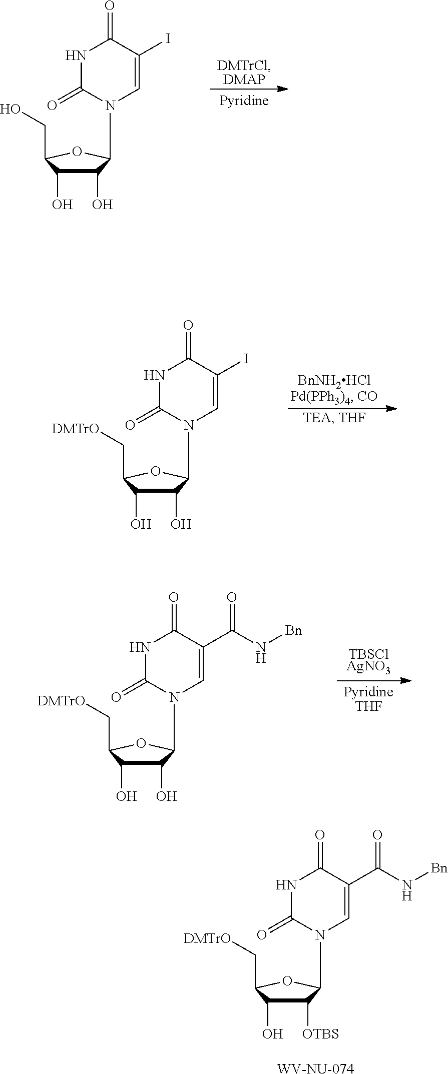

Step 1. To a solution of compound 1-((2R,3R,4S,5R)-3,4-dihydroxy-5-(hydroxymethyl)tetrahydrofuran-2-yl)-5-iodopyrimidine-2,4(1H,3H)-dione (90 g, 243.18 mmol, 1 eq.) and DMAP (2.97 g, 24.32 mmol, 0.1 eq.) in Pyridine (900 mL) was added DMTrCl (90.64 g, 267.50 mmol, 1.1 eq.). The mixture was stirred at 15° C. for 3 hr. TLC showed the reaction was complete. The reaction mixture was diluted with EtOAc (2000 mL) and washed with sat. NaHCO₃(aq., 1000 mL). The water phase was extracted with EtOAc (1000 mL*2). The combined organic layers were dried over Na₂SO₄, filtered and concentrated under reduced pressure to give a residue. The residue was purified by MPLC (SiO₂, Petroleum ether/Ethyl acetate=9/1 to 0:1, and then Ethyl acetate: MeOH=50:1, 1% TEA). 1-((2R,3R,4S,5R)-5-((bis(4-methoxyphenyl) (phenyl)methoxy)methyl)-3,4-dihydroxytetrahydrofuran-2-yl)-5-iodopyrimidine-2,4(1H,3H)-dione (132 g, 196.29 mmol, 80.72% yield) was obtained as a brown solid. ¹H NMR (400 MHz, CHLOROFORM-d) δ=8.03 (s, 1H), 7.34 (br d, J=7.6 Hz, 2H), 7.26-7.18 (m, 6H), 7.15-7.08 (m, 1H), 6.76 (d, J=8.7 Hz, 4H), 6.07 (br s, 2H), 5.85 (br d, J=4.3 Hz, 1H), 4.40 (br t, J=4.5 Hz, 1H), 4.32 (br d, J=3.9 Hz, 1H), 4.16 (br s, 1H), 3.67 (s, 6H), 3.40-3.24 (m, 2H).

Step 2. A solution of 1-((2R,3R,4S,5R)-5-((bis(4-methoxyphenyl)(phenyl)methoxy)methyl)-3,4-dihydroxytetrahydrofuran-2-yl)-5-iodopyrimidine-2,4(1H,3H)-dione (63 g, 93.69 mmol, 1 eq.), phenylmethanamine; hydrochloride (47.09 g, 327.90 mmol, 3.5 eq.), Pd(PPh₃)₄ (21.65 g, 18.74 mmol, 0.2 eq.), TEA (189.60 g, 1.87 mol, 260.80 mL, 20 eq.) in THF (630 mL) saturated with CO was stirred under 150 Psi at 70° C. for 48 hr in an autoclave. LC-MS showed starting material was disappeared. The reaction mixture was filtered and concentrated under reduced pressure to give a residue. The residue was purified by MPLC (SiO₂, Petroleum ether/Ethyl acetate=9/1 to 0:1, 1% TEA). N-benzyl-1-((2R,3R,4S,5R)-5-((bis(4-methoxyphenyl)(phenyl)methoxy)methyl)-3,4-dihydroxytetrahydrofuran-2-yl)-2,4-dioxo-1,2,3,4-tetrahydropyrimidine-5-carboxamide (25 g, 36.78 mmol, 39.26% yield) was obtained as a yellow solid. MS: 671.0 (M−H)⁻.

Step 3. To a solution of N-benzyl-1-((2R,3R,4S,5R)-5-((bis(4-methoxyphenyl)(phenyl)methoxy)methyl)-3,4-dihydroxytetrahydrofuran-2-yl)-2,4-dioxo-1,2,3,4-tetrahydropyrimidine-5-carboxamide (25 g, 36.78 mmol, 1 eq.) in THF (500 mL) was added silver nitrate (7.50 g, 44.14 mmol, 1.2 eq.), pyridine (14.55 g, 183.90 mmol, 14.84 mL, 5 eq.) and TBSCl (7.21 g, 47.81 mmol, 5.86 mL, 1.3 eq.). The mixture was stirred in dark at 15° C. for 48 hr. TLC showed starting material was consumed. The reaction mixture was added with sat. NaHCO₃(aq., 50 mL) and extracted with EtOAc (500 mL*3). The combined organic layers were dried over Na₂SO₄, filtered and concentrated under reduced pressure to give a residue. The residue was purified by MPLC (SiO₂, Petroleum ether/Ethyl acetate=9/1 to 0/1) 2 times. The residue was purified by column chromatography (SiO₂, Petroleum ether/Ethyl acetate=9/1 to 0/1) 6 times. N-benzyl-1-((2R,3R,4R,5R)-5-((bis(4-methoxyphenyl)(phenyl)methoxy)methyl)-3-((tert-butyldimethylsilyl)oxy)-4-hydroxytetrahydrofuran-2-yl)-2,4-dioxo-1,2,3,4-tetrahydropyrimidine-5-carboxamide (WV-NU-074) (4.4 g) was obtained as a yellow solid. ¹H NMR (400 MHz, CHLOROFORM-d) δ=8.75 (t, J=5.8 Hz, 1H), 8.52 (s, 1H), 7.63-7.57 (m, 9H), 7.51-7.44 (m, 5H), 7.43-7.35 (m, 12H), 7.33-7.08 (m, 30H), 6.76 (dd, J=1.1, 8.9 Hz, 5H), 5.77 (d, J=4.4 Hz, 1H), 4.56-4.44 (m, 2H), 4.37 (d, J=5.8 Hz, 3H), 4.31-4.26 (m, 1H), 4.01-3.90 (m, 2H), 3.69 (s, 7H), 3.45-3.30 (m, 2H), 0.85-0.80 (m, 9H), 0.00 (d, J=3.3 Hz, 6H); MS: 792.3 (M−H⁺).

### Example 30. Synthesis of 1-((2R,3R,4R,5R)-5-((bis(4-methoxyphenyl)(phenyl)methoxy)methyl)-3-((tert-butyldimethylsilyl)oxy)-4-hydroxytetrahydrofuran-2-yl)-N-methyl-2,4-dioxo-1,2,3,4-tetrahydropyrimidine-5-carboxamide

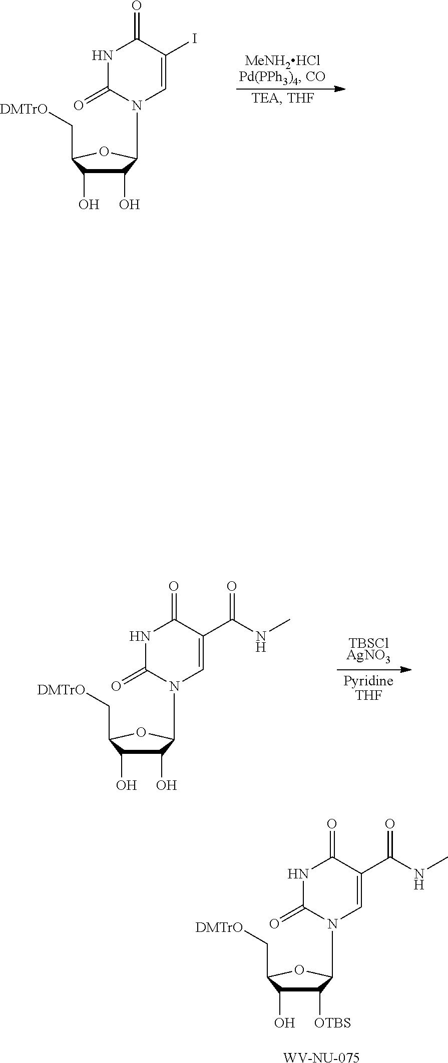

Step 1. A solution of 1-((2R,3R,4S,5R)-5-((bis(4-methoxyphenyl)(phenyl)methoxy)methyl)-3,4-dihydroxytetrahydrofuran-2-yl)-5-iodopyrimidine-2,4(1H,3H)-dione (63 g, 93.69 mmol, 1 eq.), methylamine; hydrochloride (63.26 g, 936.85 mmol, 10 eq.), Pd(PPh₃)₄ (21.65 g, 18.74 mmol, 0.2 eq.), TEA (189.60 g, 1.87 mol, 260.80 mL, 20 eq.) in THF (500 mL) saturated with CO was stirred under 150 Psi at 70° C. for 48 hr in an autoclave. LC-MS showed starting material was consumed and one main peak with desired MS was detected. The reaction mixture was filtered and concentrated under reduced pressure to give a residue. TLC (Petroleum ether:Ethyl acetate=0:1, 5% TEA, run 3 times, R_{f}=0.30). The residue was purified by MPLC (SiO₂, Petroleum ether/Ethyl acetate=9/1 to 0:1, 1% TEA) for 3 times. 1-((2R,3R,4S,5R)-5-((bis(4-methoxyphenyl) (phenyl)methoxy)methyl)-3,4-dihydroxytetrahydrofuran-2-yl)-N-methyl-2,4-dioxo-1,2,3,4-tetrahydropyrimidine-5-carboxamide (13.6 g, 22.53 mmol, 24.05% yield) was obtained as a yellow solid. MS: 602.1 (M−H)⁻.

Step 2. To a solution of 1-((2R,3R,4S,5R)-5-((bis(4-methoxyphenyl)(phenyl)methoxy)methyl)-3,4-dihydroxytetrahydrofuran-2-yl)-5-iodopyrimidine-2,4(1H,3H)-dione (13.6 g, 22.53 mmol, 1 eq.) in THF (260 mL) was added silver nitrate (4.59 g, 27.04 mmol, 1.2 eq.), pyridine (8.91 g, 112.65 mmol, 9.09 mL, 5 eq.) and TBSCl (4.41 g, 29.29 mmol, 3.59 mL, 1.3 eq.). The mixture was stirred at 15° C. for 48 hr. TLC (Petroleum ether:Ethyl acetate=1:1, R_{f}=0.52 and 0.65) showed starting material was consumed and one desired spot was detected. The reaction mixture was added with sat. NaHCO₃(aq., 100 mL) and extracted with EtOAc (300 mL*3). The combined organic layers were dried over Na₂SO₄, filtered and concentrated under reduced pressure to give a residue. TLC (Petroleum ether:Ethyl acetate=1:4, 5% TEA, R_{f}=0.10 and 0.20). The residue was purified by MPLC (SiO₂, Petroleum ether/Ethyl acetate=9/1 to 0/1, 1% TEA). 1-((2R,3R,4R,5R)-5-((bis(4-methoxyphenyl)(phenyl)methoxy)methyl)-3-((tert-butyldimethylsilyl)oxy)-4-hydroxytetrahydrofuran-2-yl)-N-methyl-2,4-dioxo-1,2,3,4-tetrahydropyrimidine-5-carboxamide (WV-NU-075) (10.5 g) was obtained as a white solid. ¹H NMR (400 MHz, CHLOROFORM-d) δ=8.52 (d, J=15.8 Hz, 2H), 8.43-8.26 (m, 2H), 7.61 (dd, J=7.9, 11.9 Hz, 1H), 7.50-7.34 (m, 5H), 7.33-7.08 (m, 15H), 6.76 (br d, J=8.3 Hz, 8H), 5.75 (dd, J=4.4, 15.6 Hz, 1H), 4.32-4.11 (m, 2H), 4.01-3.90 (m, 2H), 3.77-3.63 (m, 12H), 3.43-3.16 (m, 4H), 2.97-2.76 (m, 6H), 0.87-0.81 (m, 9H), 0.79-0.73 (m, 9H), 0.10-−0.02 (m, 6H),−0.04-−0.10 (m, 3H),−0.12-−0.19 (m, 3H); MS: 716.3 (M−H)⁺.

### Example 31. Synthesis of 1-((2R,4S,5R)-5-((bis(4-methoxyphenyl)(phenyl)methoxy)methyl)-4-hydroxytetrahydrofuran-2-yl)tetrahydropyrimidin-2(1H)-one

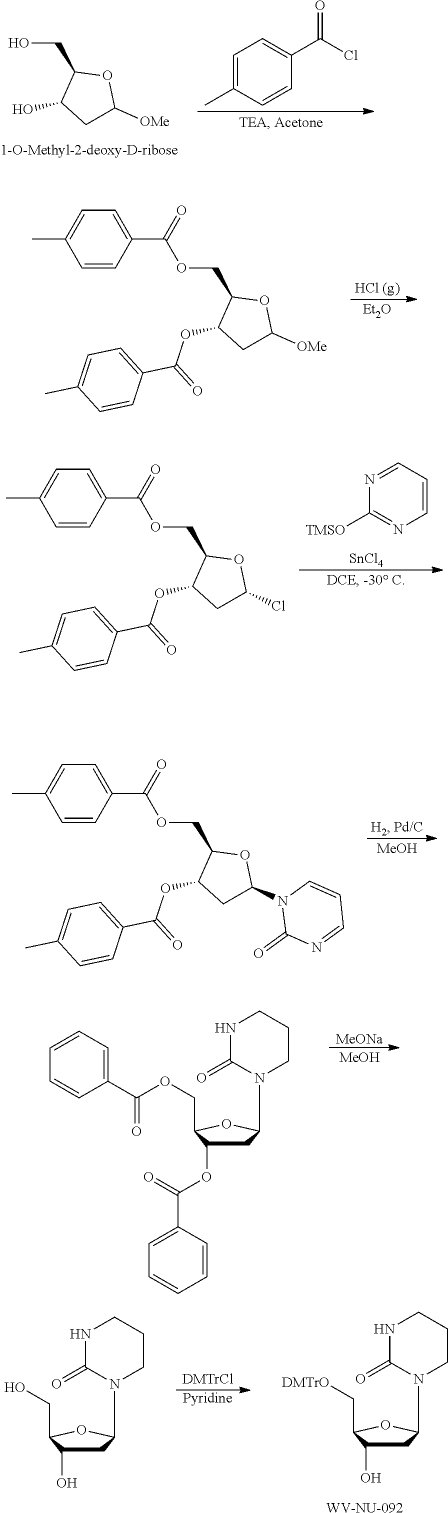

Step 1. 4-methylbenzoyl chloride (93.91 g, 607.46 mmol, 80.27 mL, 2 eq.) was dissolved with the 1-O-Methyl-2-deoxy-D-ribose (45 g, 303.73 mmol, 36.89 mL, 1 eq.) in acetone (500 mL) and then Et₃N (67.62 g, 668.21 mmol, 93.01 mL, 2.2 eq.) was added slowly make sure the temperature was blow 20° C., and the mixture was stirred at 15° C. for 16 hr. LCMS and TLC (Petroleum ether/Ethyl acetate=3:1) showed 1-O-Methyl-2-deoxy-D-ribose was consumed and the desired substance was found. The mixture was filtered and the filtrated was evaporated to get the crude. The residue was purified by column chromatography (SiO₂, Petroleum ether/Ethyl acetate=30/1 to 10/1) for three times to get (2R,3S)-5-methoxy-2-(((4-methylbenzoyl)oxy)methyl)tetrahydrofuran-3-yl 4-methylbenzoate (70 g, 182.09 mmol, 59.95% yield) as a yellow solid. ¹HNMR (400 MHz, CHLOROFORM-d) δ=8.05-7.92 (m, 4H), 7.31-7.25 (m, 4H), 5.68-5.42 (m, 1H), 5.31-5.21 (m, 1H), 4.71-4.48 (m, 3H), 3.47 (s, 2H), 3.41 (s, 2H), 2.66-2.53 (m, 1H), 2.47-2.37 (m, 7H); MS: 407.2 (M+Na)⁺.

Step 2. HCl (gas) was bubbled into a solution of compound (2R,3S)-5-methoxy-2-(((4-methylbenzoyl)oxy)methyl)tetrahydrofuran-3-yl 4-methylbenzoate (33 g, 85.84 mmol, 1 eq.) in anhydrous Et₂O (600 mL) at 0° C. for 1 hr. TLC (Petroleum ether:Ethyl acetate=3:1, Rf=0.21) showed starting material was consumed. The solid was filtered and washed with dry diethyl ether (2*150 mL), and the cake was dried over high vacuum to get (2R,3S,5R)-5-chloro-2-(((4-methylbenzoyl)oxy)methyl)tetrahydrofuran-3-yl 4-methylbenzoate (26 g, 66.87 mmol, 77.89% yield) as a white solid and without any further purification. ¹H NMR (400 MHz, CHLOROFORM-d) δ=8.02 (d, J=8.1 Hz, 2H), 7.92 (d, J=8.1 Hz, 2H), 7.36-7.20 (m, 4H), 6.50 (d, J=5.0 Hz, 1H), 5.58 (dd, J=2.4, 7.1 Hz, 1H), 4.88 (q, J=3.3 Hz, 1H), 4.75-4.55 (m, 2H), 2.89 (ddd, J=5.3, 7.4, 15.0 Hz, 1H), 2.77 (d, J=15.0 Hz, 1H), 2.44 (d, J=4.8 Hz, 5H); TLC (Petroleum ether:Ethyl acetate=3:1), Rf=0.21.

Step 3. To a solution of (2R,3S,5R)-5-chloro-2-(((4-methylbenzoyl)oxy)methyl)tetrahydrofuran-3-yl 4-methylbenzoate (26 g, 66.87 mmol, 1 eq.), Molecular sieve 4A (10 g, 66.87 mmol, 1 eq.) and compound 2-((trimethylsilyl)oxy)pyrimidine (38.08 g, 226.31 mmol, 4 eq.) in DCE (900 mL) was added SnCl₄ (24.56 g, 94.28 mmol, 11.01 mL, 1.41 eq.) in DCE (100 mL) at −30° C. The mixture was stirred at −30° C. for 5 hr. TLC showed starting material was consumed. The mixture was concentrated to get the crude. The residue was purified by column chromatography (SiO₂, Petroleum ether/Ethyl acetate=10/1 to 0/1) to get 50 g crude, and re-purified by RP-HPLC to get ((2R,3S,5R)-3-((4-methylbenzoyl)oxy)-5-(2-oxopyrimidin-1(2H)-yl)tetrahydrofuran-2-yl)methyl 4-methylbenzoate (19 g, 42.37 mmol, 63.36% yield) as a white solid. ¹H NMR (400 MHz, CHLOROFORM-d) 6=8.57 (dd, J=2.9, 4.0 Hz, 1H), 8.17 (dd, J=2.8, 6.8 Hz, 1H), 7.97 (d, J=8.3 Hz, 2H), 7.83 (d, J=8.3 Hz, 2H), 7.34-7.20 (m, 5H), 6.40-6.23 (m, 2H), 5.62 (br d, J=6.4 Hz, 1H), 4.86-4.75 (m, 1H), 4.74-4.64 (m, 2H), 3.22 (ddd, J=1.8, 5.6, 14.6 Hz, 1H), 2.44 (d, J=10.6 Hz, 7H), 2.35-2.23 (m, 1H); TLC (Petroleum ether:Ethyl acetate=1:1) Rf=0.05.

Step 4. To a solution of ((2R,3S,5R)-3-((4-methylbenzoyl)oxy)-5-(2-oxopyrimidin-1(2H)-yl)tetrahydrofuran-2-yl)methyl 4-methylbenzoate (20 g, 44.60 mmol, 1 eq.) in MeOH (400 mL) was added Pd/C (5 g, 10% purity). The suspension was degassed under vacuum and purged with H₂ several times. The mixture was stirred under H₂ (20 psi) at 45° C. for 12 hours. LCMS showed starting material was consumed. The reaction mixture was filtered and the filter was concentrated to get ((2R,3S,5R)-3-(benzoyloxy)-5-(2-oxotetrahydropyrimidin-1(2H)-yl)tetrahydrofuran-2-yl)methyl benzoate (20 g, crude) as a yellow oil. ¹HNMR (400 MHz, CHLOROFORM-d) δ=7.97-7.89 (m, 4H), 7.27-7.22 (m, 4H), 6.68-6.53 (m, 1H), 5.56-5.41 (m, 1H), 5.04 (br s, 1H), 4.71-4.28 (m, 4H), 3.46-3.07 (m, 5H), 2.47-2.39 (m, 7H), 2.38-1.80 (m, 5H); MS: (M+H⁺) 453.2

Step 5. A solution of ((2R,3S,5R)-3-(benzoyloxy)-5-(2-oxotetrahydropyrimidin-1(2H)-yl)tetrahydrofuran-2-yl)methyl benzoate (20 g, 44.20 mmol, 1 eq.) in NaOMe (9.55 g, 176.80 mmol, 4 eq.) dissolved in MEOH (900 mL), the mixture was stirred at 15° C. for 3.5 hr. The mixture was concentrated to get 1-((2R,4S,5R)-4-hydroxy-5-(hydroxymethyl)tetrahydrofuran-2-yl)tetrahydropyrimidin-2(1H)-one (9.5 g, crude) as a yellow oil. MS: 217.1 (M+H)⁺.

Step 6. To a solution of compound 7 (9.5 g, 43.93 mmol, 1 eq.) in pyridine (80 mL) was added DMTCl (16.37 g, 48.33 mmol, 1.1 eq.), the mixture was stirred at 15° C. for 4 hr. LCMS showed compound 7 was consumed and the desired substance was found. The mixture was added sat.NaHCO₃(aq, 300 mL) and extracted with Ethyl acetate (200 mL*2), dried over Na₂SO₄, filtered and concentrated to get the crude. The mixture purified by silica gel chromatography (Petroleum ether/Ethyl acetate=20:1, 0:1, 5% TEA) 3 times to get 8 g. The 2 g crude mixture was re-purified by pre-HPLC column: Agela DuraShell C18 250*50 mm*10 um; mobile phase: [water (10 mM NH4HCO3)-ACN]; B %: 25%-50%, 25 min to get 1-((2R,4S,5R)-5-((bis(4-methoxyphenyl)(phenyl)methoxy)methyl)-4-hydroxytetrahydrofuran-2-yl)tetrahydropyrimidin-2(1H)-one (WV-NU-092) (4.1 g, 42% yield) as a white solid. ¹HNMR (400 MHz, CHLOROFORM-d) δ=7.45-7.40 (m, 2H), 7.34-7.28 (m, 5H), 7.24-7.19 (m, 1H), 6.83 (d, J=8.8 Hz, 4H), 6.46 (t, J=7.1 Hz, 1H), 5.31 (s, 1H), 4.80 (br s, 1H), 4.37-4.29 (m, 1H), 3.85-3.78 (m, 7H), 3.45-3.23 (m, 5H), 3.16-3.05 (m, 1H), 2.27 (d, J=4.3 Hz, 1H), 2.21-2.10 (m, 1H), 2.04-1.96 (m, 2H), 1.90-1.82 (m, 2H); MS: 517.3 (M−H)⁻.

### Example 32. Synthesis of 1-((2R,4S,5R)-5-((bis(4-methoxyphenyl)(phenyl)methoxy)methyl)-4-hydroxytetrahydrofuran-2-yl)-2,4-dioxo-N-phenyl-1,2,3,4-tetrahydropyrimidine-5-carboxamide

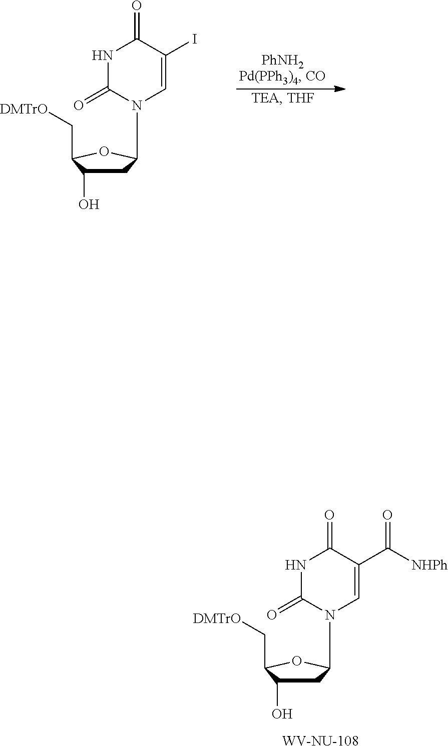

To a solution of 1-((2R,4S,5R)-5-((bis(4-methoxyphenyl)(phenyl)methoxy)methyl)-4-hydroxytetrahydrofuran-2-yl)-5-iodopyrimidine-2,4(1H,3H)-dione (30 g, 45.70 mmol, 1 eq.) in THF (300 mL) was added Pd(PPh₃)₄ (10.56 g, 9.14 mmol, 0.2 eq.), PhNH₂ (42.56 g, 456.99 mmol, 41.72 mL, 10 eq.) and TEA (92.49 g, 913.99 mmol, 127.22 mL, 20 eq.). The mixture was stirred under in CO (50 Psi) at 70° C. for 120 hr in an autoclave. The reaction mixture was concentrated under reduced pressure to remove solvent. The residue was purified by column chromatography (SiO₂, Petroleum ether/Ethyl acetate=1/0 to 0/1, then Ethyl acetate/Methanol=10/1, 5% TEA). (TLC: Ethyl acetate: Petroleum ether=2:1, R_{f}=0.20). Compound WV-NU-108 (11.5 g, 17.70 mmol, 38.73% yield) was obtained as a yellow solid. ¹H NMR (400 MHz, CHLOROFORM-d) δ=10.46 (s, 1H), 8.66 (s, 1H), 7.59 (d, J=7.8 Hz, 2H), 7.34 (d, J=7.4 Hz, 2H), 7.29 (s, 1H), 7.26-7.18 (m, 7H), 7.16-7.09 (m, 1H), 7.08-7.01 (m, 1H), 6.82-6.71 (m, 4H), 6.13 (t, J=6.4 Hz, 1H), 4.37-4.28 (m, 1H), 3.97 (d, J=4.6 Hz, 1H), 3.68 (d, J=3.1 Hz, 6H), 3.48-3.40 (m, 1H), 3.38-3.30 (m, 1H), 2.48 (ddd, J=4.1, 6.3, 13.9 Hz, 1H), 2.23 (td, J=6.6, 13.8 Hz, 1H); ¹³C NMR (101 MHz, CHLOROFORM-d) δ=162.94, 159.27, 158.59, 158.57, 149.34, 146.62, 144.53, 137.97, 135.64, 135.50, 130.11, 130.05, 128.95, 128.07, 127.96, 126.97, 124.34, 120.35, 113.31, 113.28, 106.32, 86.99, 86.71, 86.21, 77.36, 77.05, 76.73, 72.41, 63.60, 60.46, 55.18, 40.74, 21.08, 14.22; MS: 648.2 (M−H⁺) 648.2.

### Example 33. Synthesis of 1-((2R,4S,5R)-5-((bis(4-methoxyphenyl)(phenyl)methoxy)methyl)-4-hydroxytetrahydrofuran-2-yl)-N-methyl-2,4-dioxo-1,2,3,4-tetrahydropyrimidine-5-carboxamide

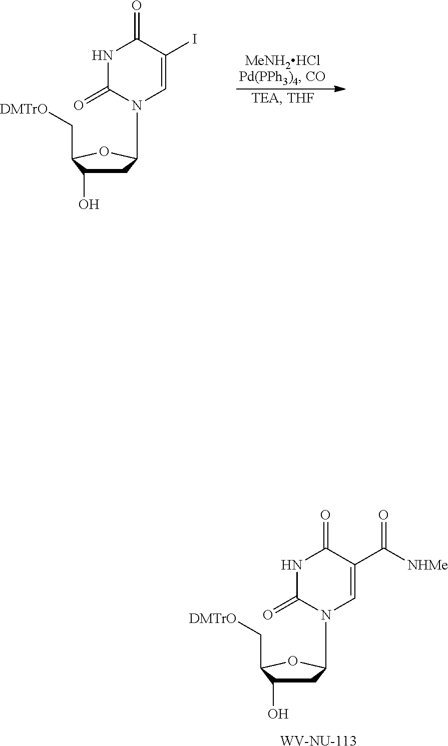

To a solution of 1-((2R,4S,5R)-5-((bis(4-methoxyphenyl)(phenyl)methoxy)methyl)-4-hydroxytetrahydrofuran-2-yl)-5-iodopyrimidine-2,4(1H,3H)-dione (30 g, 45.70 mmol, 1 eq.) in THF (300 mL) was added Pd(PPh₃)₄ (10.56 g, 9.14 mmol, 0.2 eq.), methanamine (30.86 g, 456.99 mmol, 2.78 mL, 10 eq., HCl) and TEA (92.49 g, 913.99 mmol, 127.22 mL, 20 eq.). The mixture was stirred under in CO (50 Psi) at 70° C. for 72 hr in an autoclave. LCMS showed the compound 1 was consumed and the main peak was desired. TLC: (Ethyl acetate: Petroleum ether=2:1, Rf=0.23). The reaction mixture was concentrated under reduced pressure to remove solvent. The residue was purified by column chromatography (SiO₂, Petroleum ether/Ethyl acetate=1/0 to 0/1, 5% TEA, then ethyl acetate: methanol=10/1, 5% TEA). Compound WV-NU-113 (15 g, 25.53 mmol, 55.86% yield) was obtained as a yellow solid. ¹H NMR (400 MHz, CHLOROFORM-d) δ=8.59 (s, 1H), 8.50-8.39 (m, 1H), 7.40 (d, J=7.4 Hz, 2H), 7.34-7.27 (m, 5H), 7.26 (s, 1H), 7.23-7.16 (m, 1H), 6.84 (d, J=8.9 Hz, 4H), 6.16 (s, 1H), 4.35 (br d, J=6.6 Hz, 1H), 3.97 (s, 1H), 3.79 (s, 6H), 3.47 (br d, J=4.6 Hz, 1H), 3.42-3.33 (m, 1H), 2.93 (d, J=4.9 Hz, 3H), 2.54-2.41 (m, 1H), 2.25 (s, 1H); MS: 610.2 (M+Na)⁺.

### Example 34. Synthesis of 3-((2R,4S,5R)-5-((bis(4-methoxyphenyl)(phenyl)methoxy)methyl)-4-hydroxytetrahydrofuran-2-yl)pyrimidine-2,4(1H,3H)-dione (WV-NU-096)

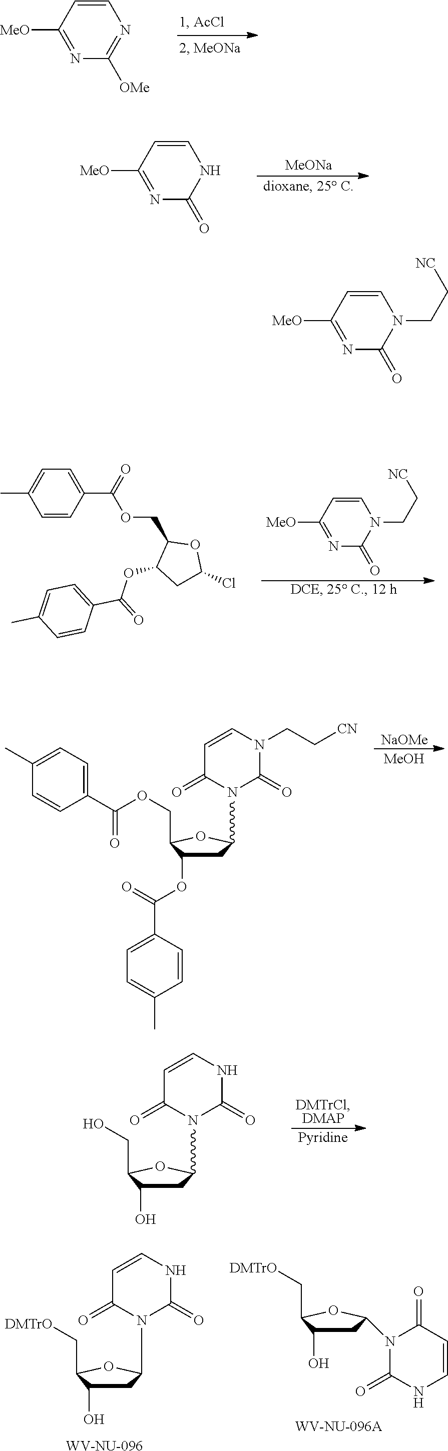

Step 1. A mixture of 2,4-dimethoxypyrimidine (25 g, 178.39 mmol, 1 eq.), acetyl chloride (38.50 g, 490.46 mmol, 35.00 mL, 2.75 eq.) was degassed and purged with N₂ for 3 times, and then the mixture was stirred at 25° C. for 24 hr under N₂ atmosphere. TLC showed 2,4-dimethoxypyrimidine was consumed completely, then to the mixture was added toluene (200 mL), and then concentrated in vacuo, repeat for 3 times. Then the residue was dissolved in MeOH (2000 mL), and then to the mixture was added sodium methoxide (10.60 g, 196.23 mmol, 1.1 eq.). The mixture was stirred at 50° C. for 1 hr. TLC indicated one new spot formed. The mixture was concentrated in vacuo, and the residue was dissolved in dioxane (1000 mL), and then concentrated in vacuo. 4-methoxypyrimidin-2(1H)-one (89 g, crude) was obtained as a white solid.

Step 2. To a solution of 4-methoxypyrimidin-2(1H)-one (20 g, 158.59 mmol, 1 eq.) in dioxane (500 mL) and acrylonitrile (84.15 g, 1.59 mol, 105.19 mL, 10 eq.) was added a solution of NaOMe (1 M, 158.59 mL, 1 eq.). The mixture was stirred at 25° C. for 12 hr. LC-MS showed 4-methoxypyrimidin-2(1H)-one was consumed completely and one main peak with desired mass was detected. The reaction was filtered, and the filtered cake was quenched by H₂O (300 mL), and then filtered, the filtrate was extracted with DCM/dioxane=50/1 (100 mL*3), and then the organic phase was dried by Na₂SO₄, and then concentrated in vacuo. 3-(4-methoxy-2-oxopyrimidin-1(2H)-yl)propanenitrile (12 g, crude) was obtained as a yellow oil. MS: 180.3 (M+H^{+). TLC (Petroleum ether:Ethyl acetate=}0: 1) R_{f1}=0.43

Step 3. To a solution of (2R,3S,5R)-5-chloro-2-(((4-methylbenzoyl)oxy)methyl)tetrahydrofuran-3-yl 4-methylbenzoate (14 g, 36.00 mmol, 1 eq.), and 3-(4-methoxy-2-oxopyrimidin-1(2H)-yl)propanenitrile (6.45 g, 36.00 mmol, 1 eq.) in DCE (200 mL) was added 4A MS (20 g). The mixture was stirred at 25° C. for 48 hr. TLC indicated starting material was consumed completely and new spots formed. The reaction was filtered, and the filtrate was concentrated in vacuo. The residue was purified by column chromatography (SiO₂, Petroleum ether/Ethyl acetate=10/1 to 5/1, 1/1, 0/1). (2R,3S)-5-(3-(2-cyanoethyl)-2,6-dioxo-3,6-dihydropyrimidin-1(2H)-yl)-2-(((4-methylbenzoyl)oxy)methyl)tetrahydrofuran-3-yl 4-methylbenzoate (1 g, crude) was obtained as a white solid. MS: 540.3 (M+Na⁺); TLC (Petroleum ether:Ethyl acetate=1:1) R_{f}=0.08.

Step 4. To a solution of (2R,3S)-5-(3-(2-cyanoethyl)-2,6-dioxo-3,6-dihydropyrimidin-1(2H)-yl)-2-(((4-methylbenzoyl)oxy)methyl)tetrahydrofuran-3-yl 4-methylbenzoate (1.5 g, 3.06 mmol, 1 eq.) in MeOH (15 mL) was added NaOMe (579.45 mg, 10.73 mmol, 3.5 eq.). The mixture was stirred at 25° C. for 12 hr. LC-MS showed starting material was consumed completely and one main peak with desired mass was detected. To the solution was added NH₄Cl (570 mg), and then the mixture was concentrated in vacuo. The residue was dissolved in pyridine (50 mL), and concentrated in high vacuo, repeated for 3 times. 3-((4S,5R)-4-hydroxy-5-(hydroxymethyl)tetrahydrofuran-2-yl)pyrimidine-2,4 (1H,3H)-dione (699 mg, crude) was obtained as a yellow oil. MS: 251.2 (M+Na)⁺.

Step 5. To a solution of 3-((4S,5R)-4-hydroxy-5-(hydroxymethyl)tetrahydrofuran-2-yl)pyrimidine-2,4(1H,3H)-dione (699 mg, 3.06 mmol, 1 eq.) in pyridine (30 mL) was added DMTrCl (1.25 g, 3.68 mmol, 1.2 eq.). The mixture was stirred at 25° C. for 12 hr. LC-MS showed starting material was consumed completely and two peaks with desired mass was detected. The mixture was concentrated in vacuo. The residue was quenched by sat. aq. NaHCO₃ (50 mL) and then extracted with EtOAc (20 mL*3). The combined organic phase was washed with brine (50 mL), dried over anhydrous Na₂SO₄, filtered and concentrated in vacuo. The residue was purified by prep-HPLC (neutral condition). Column: Agela DuraShell C18 250*50 mm*10 um; mobile phase: [water (10 mM NH₄HCO₃)-ACN]; B %: 42%-62%, 22 min. 3-((2R,4S,5R)-5-((bis(4-methoxyphenyl) (phenyl)methoxy)methyl)-4-hydroxytetrahydrofuran-2-yl)pyrimidine-2,4(1H,3H)-dione (WV-NU-096) (500 mg, 892.35 umol, 29.13% yield, 94.69% purity) was obtained as a yellow solid. 3-((2S,4S,5R)-5-((bis(4-methoxyphenyl)(phenyl)methoxy)methyl)-4-hydroxytetrahydrofuran-2-yl)pyrimidine-2,4(1H,3H)-dione (WV-NU-096A) (360 mg, 569.62 umol, 18.60% yield, 83.951% purity) was obtained as a white solid.

WV-NU-096: ¹H NMR (400 MHz, CHLOROFORM-d) δ=9.84 (br s, 1H), 7.44 (br d, J=7.8 Hz, 2H), 7.33 (d, J=8.8 Hz, 4H), 7.26-7.22 (m, 2H), 7.22-7.15 (m, 1H), 6.80 (dd, J=1.8, 8.6 Hz, 4H), 6.72-6.58 (m, 2H), 5.54 (d, J=7.6 Hz, 1H), 4.64 (q, J=6.1 Hz, 1H), 3.96-3.86 (m, 1H), 3.84-3.67 (m, 6H), 3.52-3.41 (m, 1H), 3.35 (dd, J=5.9, 9.3 Hz, 1H), 2.90-2.74 (m, 1H), 2.24-2.10 (m, 1H); ¹³C NMR (101 MHz, CHLOROFORM-d) δ=162.84, 158.45, 152.27, 144.82, 138.95, 136.00, 135.99, 129.14, 128.79, 128.16, 127.82, 130.07, 126.82, 113.12, 102.22, 86.38, 84.94, 81.13, 73.40, 64.67, 55.21, 55.19, 37.19; MS: 529.2 (M−H⁺); TLC (Petroleum ether:Ethyl acetate=1:1) R_{f}=0.14.

WV-NU-096A: ¹H NMR (400 MHz, CHLOROFORM-d) δ=8.72 (br s, 1H), 7.44 (d, J=7.6 Hz, 2H), 7.37-7.27 (m, 7H), 7.24-7.16 (m, 2H), 7.12 (br d, J=7.9 Hz, 1H), 5.76 (d, J=7.5 Hz, 1H), 5.14 (br d, J=11.5 Hz, 1H), 4.40 (br s, 1H), 4.37-4.29 (m, 1H), 3.80 (s, 6H), 3.25 (dd, J=3.9, 9.9 Hz, 1H), 3.11 (dd, J=3.9, 10.0 Hz, 1H), 2.93 (td, J=8.9, 14.8 Hz, 1H), 2.26 (br dd, J=2.9, 14.8 Hz, 1H); ¹³C NMR (101 MHz, CHLOROFORM-d) δ=163.22, 158.62, 158.45, 151.82, 144.72, 138.62, 135.96, 135.81, 130.03, 130.01, 129.11, 128.10, 127.86, 127.74, 127.06, 126.77, 113.16, 102.86, 88.75, 86.36, 83.63, 74.29, 64.76, 55.19, 39.69; LCMS: M−H⁺=529.2, purity: 83.95%; TLC (Petroleum ether:Ethyl acetate=1:1) R_{f}=0.23.

### Example 35. Synthesis of (2R,3S,5R)-5-(3-benzoyl-2-oxotetrahydropyrimidin-1(2H)-yl)-2-((bis(4-methoxyphenyl)(phenyl)methoxy)methyl)tetrahydrofuran-3-yl (2-cyanoethyl) diisopropylphosphoramidite

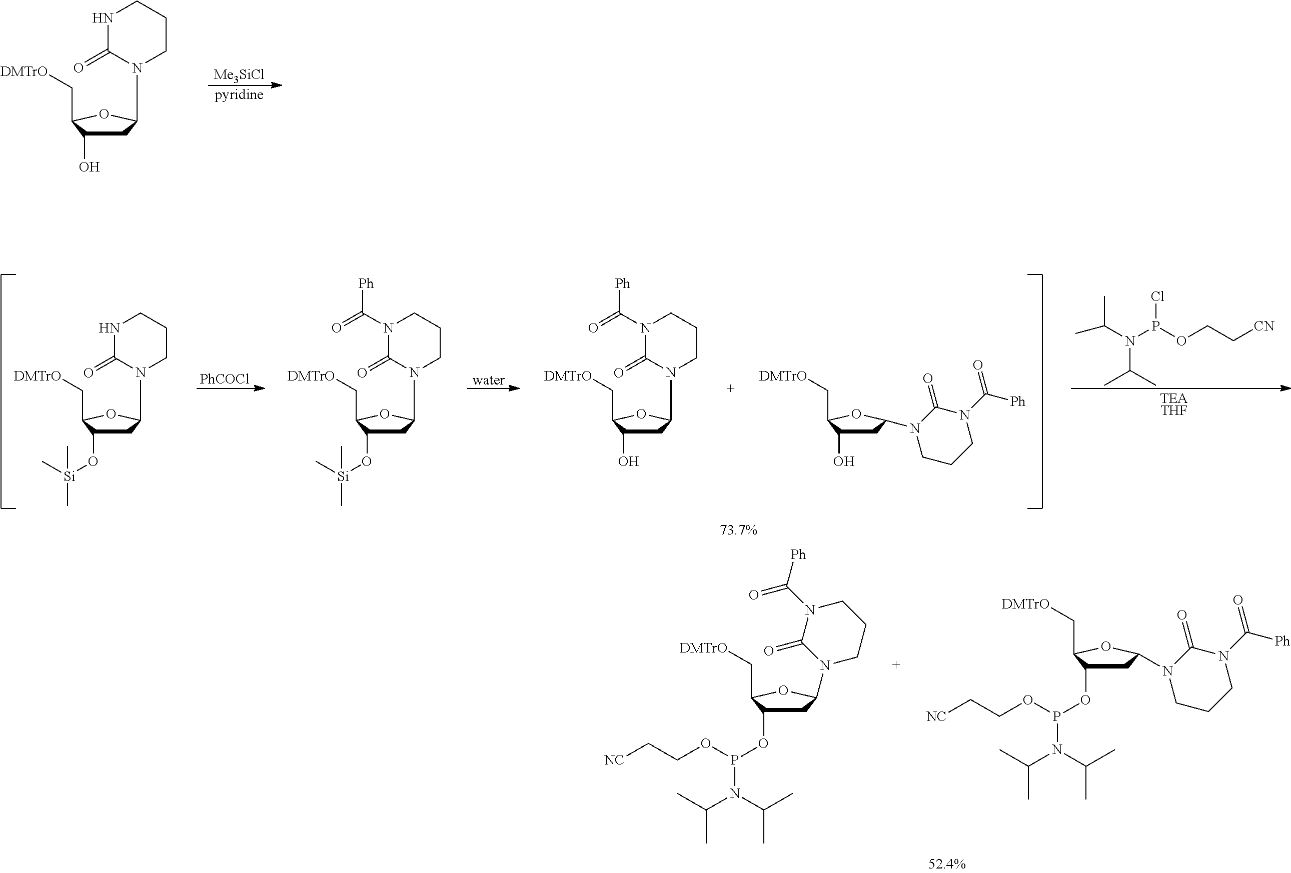

Step 1: a solution of 1-((2R,4S,5R)-5-((bis(4-methoxyphenyl)(phenyl)methoxy)methyl)-4-hydroxytetrahydrofuran-2-yl)tetrahydropyrimidin-2(1H)-one (1.17 g, 2.26 mmol) in pyridine (10.2 mL) was treated with Chlorotrimethylsilane (1.43 mL, 11.31 mmol). Stirred at rt for 1.5 hr. TLC and LC-MS showed the expected intermediate was formed. Cooled to 0° C. To the reaction solution was added benzoyl chloride (288.73 uL, 2.49 mmol) dropwise. Continued to stir first at 0° C. for 15 min, then at rt overnight. LC-MS showed the expected intermediate was observed. Iced water (3.9 mL) was added. Continued to stir for 2 hr. LC-MS showed the reaction was complete. The reaction solution was diluted with brine (20 mL) then extracted with EtOAc (3×60 mL). The combined organics were dried over sodium sulfate, filtered, and concentrated. The resulting crude product was purified by normal phase column chromatography applying 25-100% EtOAc in hexane (each mobile phase contained 5% triethylamine) as the gradient to afford a 3:2 ratio mixture of the desired product 1-benzoyl-3-((2R,4S,5R)-5-((bis(4-methoxyphenyl)(phenyl)methoxy)methyl)-4-hydroxytetrahydrofuran-2-yl)tetrahydropyrimidin-2(1H)-one (Base up) and the unwanted diastereomer (Base down) as a white foam (1.04 g, 73.7% yield). ¹H NMR (600 MHz, DMSO-d₆) δ 7.50-7.15 (m, 14H), 6.93-6.83 (m, 4H), 6.03 (q, J=6.8, 5.9 Hz, 1H), 5.23 (d, J=3.9 Hz, 0.41H, OH proton from the unwanted diastereomer), 5.15 (d, J=4.6 Hz, 0.66H, OH proton from the desired product), 4.15-4.07 (m, 1H), 3.79-3.59 (m, 8H), 3.57-3.48 (m, 1H), 3.44-3.37 (m, 1H), 3.31-3.26 (m, 1H), 3.14-3.06 (m, 1H), 3.01-2.87 (m, 1H), 2.12-2.06 (m, 1H), 2.05-1.99 (m, 1H), 1.95-1.86 (m, 1H), 1.86-1.79 (m, 1H); MS (ESI), 645.67 [M+Na]⁺.

### Step 2: To a solution of a 3:2 ratio dry mixture of 1-benzoyl-3-((2R,4S,5R)-5-((bis(4-methoxyphenyl)(phenyl)methoxy)methyl)-4-hydroxytetrahydrofuran-2-yl)tetrahydropyrimidin-2(1H)-one

(Base up) and the unwanted diastereomer (Base down) (1.3 g, 2.09 mmol) in THF (11 mL) was added triethylamine (1.05 mL, 7.52 mmol). Cooled to 0° C. To the reaction solution was added 2-Cyanoethyl N,N-diisopropylchlorophosphoramidite (0.56 mL, 2.51 mmol) dropwise in a period of 4 min. The reaction solution was continued to stir first at 0° C. for 1 hr, then at rt for 2 hr. LC-MS showed product was formed. Triethylamine (1 mL) was added followed by MgSO₄ (0.3 g) and EtOAc (20 mL). Stirred for 2 min. The mixture was filtered through a fritted funnel. The solid in the funnel was rinsed with EtOAc (20 mL). The filtrate was concentrated. The resulting crude amidite was purified by normal phase column chromatography applying 20-60% EtOAc in hexane (each mobile phase contained 2.5% triethylamine) to afford a 3:2 ratio mixture of the desired (2R,3S,5R)-5-(3-benzoyl-2-oxotetrahydropyrimidin-1(2H)-yl)-2-((bis(4-methoxyphenyl)(phenyl)methoxy)methyl)tetrahydrofuran-3-yl (2-cyanoethyl) diisopropylphosphoramidite and the unwanted diastereomer (Base down) as a white foam (0.90 g, 52.4% yield). ³¹P NMR (162 MHz, Chloroform-d) δ 148.86 (s, from one of the two unwanted stereoisomers, Base down), 148.72 (s, from the other of the two unwanted stereoisomers, Base down), 148.48 (s, from one of the two desired stereoisomers, Base up), 148.38 (s, from the other of the two desired stereoisomers, Base up); MS (ESI), 823.47 [M+H]⁺.

### Example 36. Synthesis of (2R,3S,5R)-2-((bis(4-methoxyphenyl)(phenyl)methoxy)methyl)-5-(2,4-dioxo-5-(phenylcarbamoyl)-3,4-dihydropyrimidin-1(2H)-yl)tetrahydrofuran-3-yl (2-cyanoethyl) diisopropylphosphoramidite

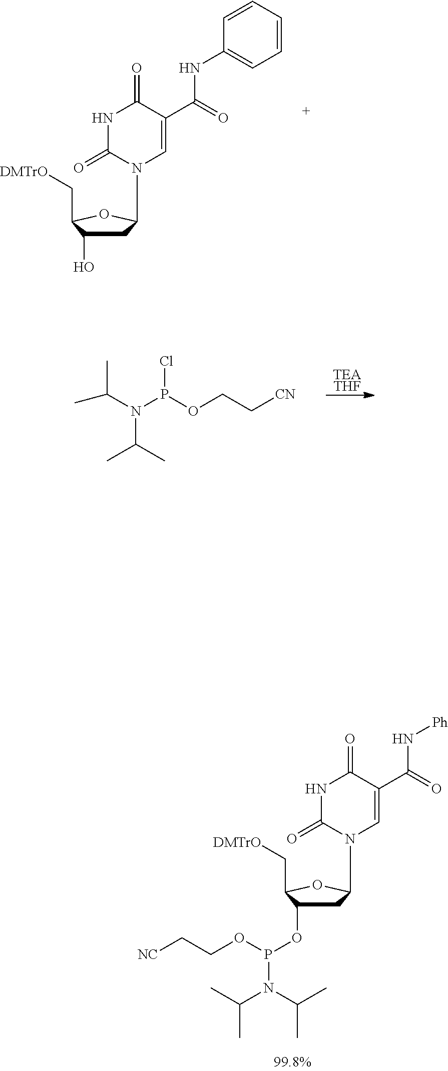

To a solution of dry 1-((2R,4S,5R)-5-((bis(4-methoxyphenyl)(phenyl)methoxy)methyl)-4-hydroxytetrahydrofuran-2-yl)-2,4-dioxo-N-phenyl-1,2,3,4-tetrahydropyrimidine-5-carboxamide (2.0 g, 3.08 mmol) in THF (17.4 mL) was added triethylamine (1.72 mL, 12.31 mmol). Cooled to 0° C. To the solution was added 2-Cyanoethyl N,N-diisopropylchlorophosphoramidite (0.89 mL, 4.0 mmol) dropwise in a period of 5 min. The reaction solution was continued to stir first at 0° C. for 30 min, then at rt for 3.5 hr. TLC showed the reaction was complete. Triethylamine (1.72 mL) was added followed by anhydrous MgSO₄ (0.55 g) and EtOAc (25 mL). Stirred for 2 min. The mixture was filtered. The solid in the fritted funnel was rinsed with EtOAc (25 mL). The filtrate was concentrated. The resulting crude product was purified by normal phase column chromatography applying 20-100% EtOAc in hexane (each mobile phase contained 5% triethylamine) as the gradient to afford (2R,3S,5R)-2-((bis(4-methoxyphenyl)(phenyl)methoxy)methyl)-5-(2,4-dioxo-5-(phenylcarbamoyl)-3,4-dihydropyrimidin-1(2H)-yl)tetrahydrofuran-3-yl (2-cyanoethyl) diisopropylphosphoramidite as a white foam (2.61 g, 99.8% yield). ¹H NMR (600 MHz, Chloroform-d) δ 10.64 (bs, 1H), 8.78-8.72 (m, 1H), 7.65 (d, J=8.0 Hz, 2H), 7.41 (t, J=6.5 Hz, 2H), 7.33 (q, J=8.0 Hz, 6H), 7.29-7.24 (m, 3H), 7.18 (q, J=7.4 Hz, 1H), 7.10 (t, J=7.4 Hz, 1H), 6.83 (q, J=7.3, 6.7 Hz, 4H), 6.20 (q, J=7.3 Hz, 1H), 4.50-4.42 (m, 1H), 4.26-4.19 (m, 1H), 3.88-3.77 (m, 1H), 3.70-3.61 (m, 1H), 3.61-3.50 (m, 2H), 3.42-3.32 (m, 2H), 2.78-2.65 (m, 1H), 2.45 (t, J=6.7 Hz, 1H), 2.32-2.24 (m, 2H), 1.27 (t, J=7.7 Hz, 3H), 1.20-1.13 (m, 9H); ³¹P NMR (243 MHz, Chloroform-d) δ 149.19, 148.80; MS (ESI), 848.68 [M−H]⁻.

### Example 37. Synthesis of 9-((2R,4S,5R)-5-((bis(4-methoxyphenyl)(phenyl)methoxy)methyl)-4-(((1S,3S,3aS)-3-((methyldiphenylsilyl)methyl)tetrahydro-1H,3H-pyrrolo[1,2-c][1,3,2]oxazaphosphol-1-yl)oxy)tetrahydrofuran-2-yl)-1,9-dihydro-6H-purin-6-one

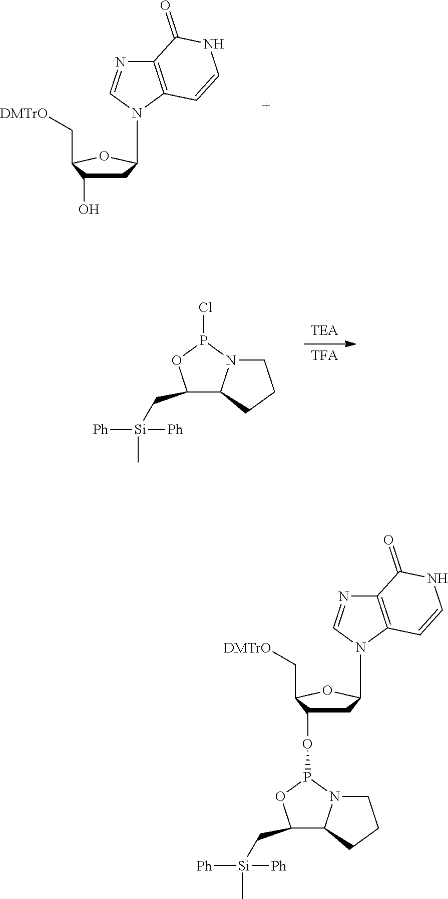

Dry 9-((2R,4S,5R)-5-((bis(4-methoxyphenyl)(phenyl)methoxy)methyl)-4-hydroxytetrahydro-furan-2-yl)-1,9-dihydro-6H-purin-6-one (7.5 g, 13.52 mmol) was poorly dissolved in THF (500 mL). Triethylamine (9.42 mL, 67.62 mmol) was added. The reaction flask was set in a water bath. (3S,3aS)-1-chloro-3-((methyldiphenylsilyl)methyl)tetrahydro-1H,3H-pyrrolo[1,2-c][1,3,2]oxazaphosphole (0.9574M in THF, 19.78 mL, 18.93 mmol) was added in a period of 8 min. The water bath was removed, and the reaction slurry was stirred at rt for 5 hr. TLC and LC-MS showed the reaction was not complete due to poor solubility of the substrate. Triethylamine (9.4 mL) was added followed by MgSO₄ (6.0 g) and EtOAc (150 mL). The mixture was filtered through a fritted funnel filled with a thin layer of celite. The solid in the funnel was rinsed with EtOAc (100 mL). The filtrate was concentrated. The resulting crude product was purified by normal phase column chromatography applying 0-100% ACN in EtOAc (each mobile phase contained 5% triethylamine) as the gradient to afford 9-((2R,4S,5R)-5-((bis(4-methoxyphenyl)(phenyl)methoxy)methyl)-4-(((1S,3S,3aS)-3-((methyldiphenyl silyl)methyl)tetrahydro-1H,3H-pyrrolo[1,2-c][1,3,2]oxazaphosphol-1-yl)oxy)tetrahydrofuran-2-yl)-1,9-dihydro-6H-purin-6-one as a white foamy solid (2.4 g, 19.9% yield). ¹H NMR (400 MHz, Chloroform-d) δ 7.96 (s, 1H), 7.90 (s, 1H), 7.52-7.17 (m, 19H), 6.84-6.74 (m, 4H), 6.31-6.24 (m, 1H), 4.89-4.81 (m, 1H), 4.77 (dt, J=10.1, 5.6 Hz, 1H), 4.07-4.01 (m, 1H), 3.76 (s, 6H), 3.54 (ddd, J=18.2, 14.9, 7.8 Hz, 1H), 3.36 (dq, J=12.9, 5.9 Hz, 1H), 3.26 (d, J=4.4 Hz, 2H), 3.09 (qd, J=10.8, 4.1 Hz, 1H), 2.48 (dt, J=13.7, 6.7 Hz, 1H), 2.37-2.27 (m, 1H), 1.90-1.83 (m, 1H), 1.75-1.65 (m, 1H), 1.56 (dd, J=14.7, 9.0 Hz, 1H), 1.43 (dd, J=14.2, 6.0 Hz, 2H), 1.31-1.21 (m, 1H), 0.63 (s, 3H); ³¹P NMR (162 MHz, Chloroform-d) δ 151.40; MS (ESI), 894.91 [M+H]⁺.

### Example 38. Synthesis of (2R,3R,4R,5R,6R)-5-acetamido-2-(acetoxymethyl)-64(5-((2,5-dioxopyrrolidin-1-yl)oxy)-5-oxopentyl)oxy)tetrahydro-2H-pyran-3,4-diyl diacetate

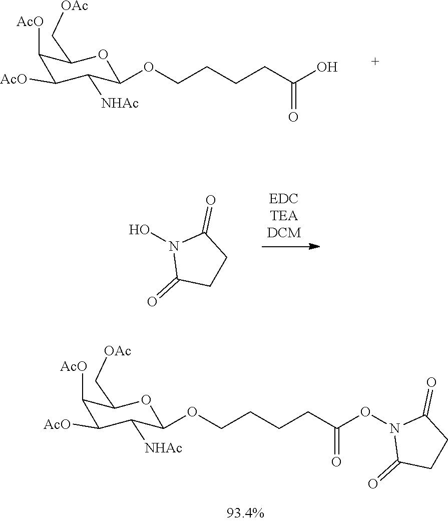 [SMILES: CC(=O)NC1C(OCCCCC(=O)O)OC(COC(C)=O)C(OC(C)=O)C1OC(C)=O ; O=C1CCC(=O)N1O ; CC(=O)NC1C(OCCCCC(=O)ON2C(=O)CCC2=O)OC(COC(C)=O)C(OC(C)=O)C1OC(C)=O]

To a solution of 5-(((2R,3R,4R,5R,6R)-3-acetamido-4,5-diacetoxy-6-(acetoxymethyl)tetrahydro-2H-pyran-2-yl)oxy)pentanoic acid (36.0 g, 80.46 mmol) in DCM (450 mL) was added N-Hydroxysuccinimide (13.89 g, 120.69 mmol) followed by triethylamine (33.6 mL, 241 mmol). The solution was cooled to 0° C. EDCHCl (30.8 g, 160.9 mmol) was added in portions. Continued to stir first at 0° C. for 5 min, then at rt overnight. LC-MS showed the reaction was complete. The volatiles were evaporated to give a cloudy oil which was suspended in EtOAc (450 mL) and washed with water (150 mL). The aqueous layer was back-extracted with EtOAc (3×225 mL). The combined organic layers were dried over Na₂SO₄, filtered, and concentrated. The resulting greasy foam was co-evaporated with DCM (3×100 mL) then further dried on high vacuum overnight to afford the product (2R,3R,4R,5R,6R)-5-acetamido-2-(acetoxymethyl)-6-((5-((2,5-dioxopyrrolidin-1-yl)oxy)-5-oxopentyl)oxy)tetrahydro-2H-pyran-3,4-diyl diacetate (40.9 g, 93.4% yield). ¹H NMR (400 MHz, Chloroform-d) δ 5.82 (d, J=8.8 Hz, 1H), 5.35 (dd, J=3.5, 1.2 Hz, 1H), 5.27 (dd, J=11.2, 3.4 Hz, 1H), 4.71 (d, J=8.4 Hz, 1H), 4.23-4.07 (m, 2H), 4.05-3.89 (m, 1H), 3.94-3.85 (m, 2H), 3.60 (ddd, J=11.0, 6.4, 4.8 Hz, 1H), 2.87 (m, 4H), 2.79-2.56 (m, 2H), 2.15 (s, 3H), 2.05 (s, 3H), 2.00 (s, 3H), 1.95 (s, 3H), 1.91-1.65 (m, 4H); MS (ESI), 545.20 [M+H]⁺.

### Example 39. Synthesis of 1-(((2R,3R,4R,5R,6R)-3-acetamido-4,5-diacetoxy-6-(acetoxymethyl)tetrahydro-2H-pyran-2-yl)oxy)-16,16-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,11,18-trioxo-14-oxa-6,10,17-triazanonacosan-29-oic acid

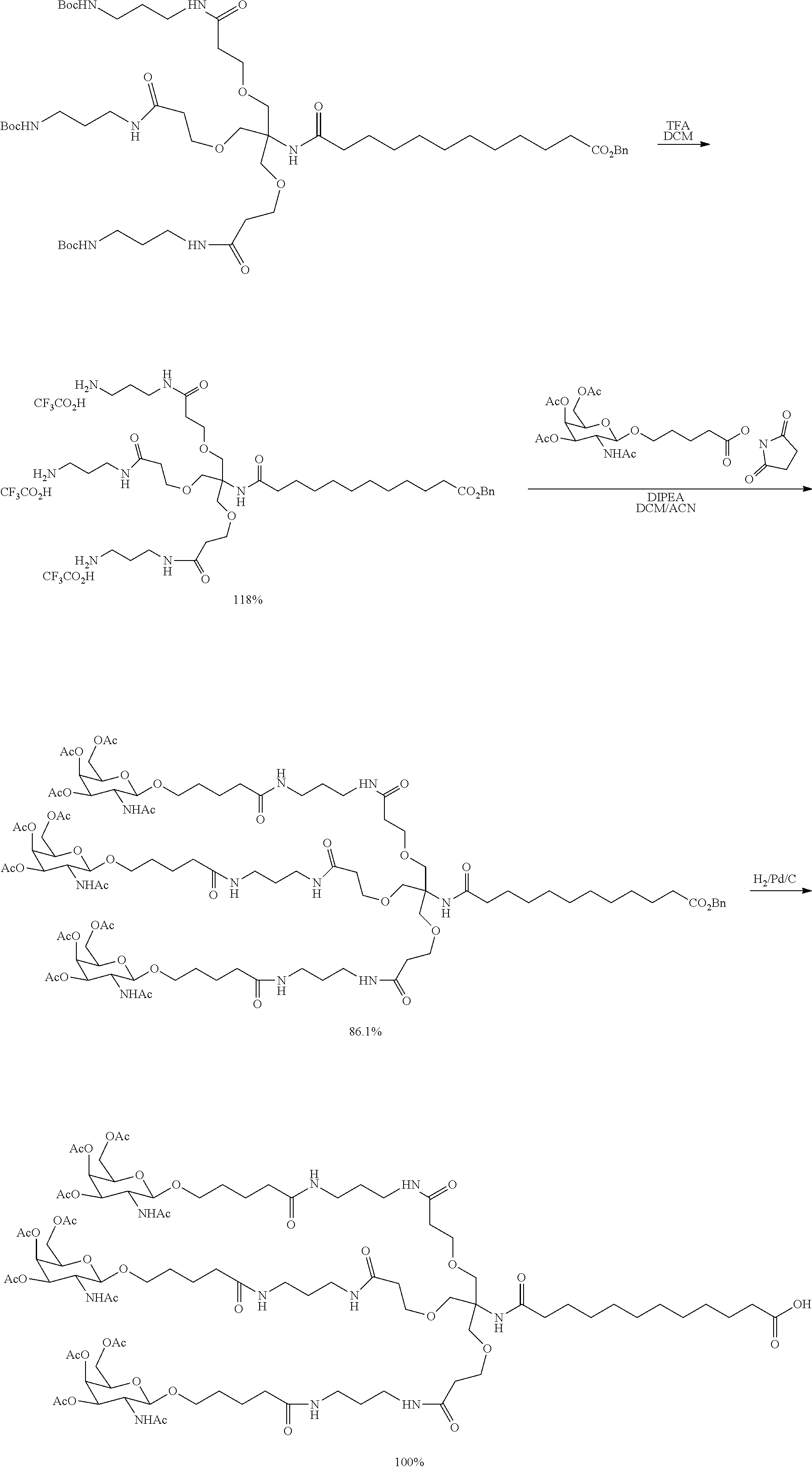

Step 1: A solution of benzyl 12-[[2-[3[-[3-(tert-butoxycarbonylamino)propylamino]-3-oxo-propoxy]-1,1-bis [3-[3-(tert-butoxycarbonylamino)propylamino]-3-oxo-propoxy]methyl]ethyl]amino]-12-oxo-dodecanoate (20.8 g, 18.765 mmol) in DCM (140 mL) was cooled to 0° C. Trifluoroacetic acid (43.4 mL, 562.96 mmol) was added in a period of 10 min. Continued to stir first at 0° C. for 15 min, then at rt for 3 hr. LC-MS showed the reaction was complete. The volatiles were evaporated. The resulting residue was co-evaporated successively with DCM (150 mL) and ACN (150 mL). The residue was re-dissolved in ACN (30 mL) and triturated with diethyl ether (300 mL). The resulting white cloudy mixture was set in a fridge overnight. The ether layer was decanted. The remaining oil was co-evaporated again with ACN (30 mL) to afford an oil. The oil was further dried on high vacuum over three nights to afford benzyl 12-((1,19-diamino-10-((3-aminopropyl)amino)-3-oxopropoxy)methyl)-5,15-dioxo-8,12-dioxa-4,16-diazanonadecan-10-yl)amino)-12-oxododecanoate tris(2,2,2-trifluoroacetate) as an oil (25.37 g, 118% yield). ¹H NMR (400 MHz, DMSO-d₆) δ 8.03 (t, J=5.8 Hz, 3H), 7.78 (bs, 9H), 7.40-7.30 (m, 5H), 6.98 (s, 1H), 5.08 (s, 2H), 3.60-3.50 (m, 12H), 3.11 (q, J=6.6 Hz, 6H), 2.84-2.70 (m, 6H), 2.38-2.28 (m, 8H), 2.05 (t, J=7.48 Hz, 2H), 1.67 (p, J=7.0 Hz, 6H), 1.57-1.48 (m, 2H), 1.46-1.39 (m, 2H), 1.27-1.17 (m, 12H); MS (ESI), 808.57 [M+H]⁺.

Step 2: To benzyl 12-((1,19-diamino-10-((3-((3-aminopropyl)amino)-3-oxopropoxy)methyl)-5,15-dioxo-8,12-dioxa-4,16-diazanonadecan-10-yl)amino)-12-oxododecanoate tris(2,2,2-trifluoroacetate) (21.5 g, 18.694 mmol) were added acetonitrile (125 mL) and DIPEA (39.1 mL, 224.33 mmol). To the resulting cloudy solution was added DCM (125 mL) and the mixture became a clear solution. A solution of (2R,3R,4R,5R,6R)-5-acetamido-2-(acetoxymethyl)-6-[5-((2,5-dioxopyrrolidin-1-yl)oxy)-5-oxopentyl)oxy)tetrahydro-2H-pyran-3,4-diyl diacetate (40.9 g, 75.113 mmol) in DCM (250 mL) was cooled to 0° C. The basified amine solution was added slowly to the cold electrophile solution in a period of 15 min. Continued to stir first at 0° C. for 10 min, then at rt for 70 min. LC-MS showed the reaction was complete. The reaction solution was concentrated. The resulting oil was co-evaporated first with ACN (2×50 mL) then with DCM (2×50 mL). The crude product was purified by normal phase column chromatography applying 0-40% MeOH in DCM as the gradient to afford benzyl 12-[[2-[3-[3-[5-[3-acetamido-4,5-diacetoxy-6-(acetoxymethyl)tetrahydropyran-2-yl]oxypentanoylamino]propylamino]-3-oxo-propoxy]-1,1-bis[[3-[3-[5-[3-acetamido-4,5-diacetoxy-6-(acetoxymethyl)tetrahydropyran-2-yl]oxypentanoylamino]propylamino]-3-oxo-propoxylmethyl]ethyl]amino]-12-oxo-dodecanoate as a white foam (33.7 g, 86.1% yield). ¹H NMR (400 MHz, DMSO-d₆) δ 7.85-7.77 (m, 6H), 7.71 (t, J=5.7 Hz, 3H), 7.42-7.28 (m, 5H), 6.96 (s, 1H), 5.21 (d, J=3.4 Hz, 3H), 5.08 (s, 2H), 4.97 (dd, J=11.2, 3.4 Hz, 3H), 4.48 (d, J=8.4 Hz, 3H), 4.07-3.96 (m, 9H), 3.87 (dt, J=11.2, 8.8 Hz, 3H), 3.71 (dd, J=10.3, 5.6 Hz, 3H), 3.58-3.50 (m, 12H), 3.40 (dt, J=9.7, 6.0 Hz, 3H), 3.03 (p, J=6.3 Hz, 12H), 2.36-2.24 (m, 8H), 2.10 (s, 9H), 2.07-2.01 (m, 8H), 1.99 (s, 9H), 1.89 (s, 9H), 1.77 (s, 9H), 1.56-1.41 (m, 22H), 1.28-1.18 (m, 12H); MS (ESI), 1048.95 [M/2+H]⁺.

Step 3: Benzyl 12-[[2-[3-[3-[5-[3-acetamido-4,5-diacetoxy-6-(acetoxymethyl)tetrahydropyran-2-yl]oxypentanoylamino]propylamino]-3-oxo-propoxy]-1,1-bis[[3-[3-[5-[3-acetamido-4,5-diacetoxy-6-(acetoxymethyl)tetrahydropyran-2-yl]oxypentanoylamino]propylamino]-3-oxo-propoxy]methyl]ethyl]amino]-12-oxo-dodecanoate (30.8 g, 14.692 mmol) was dissolved in EtOAc (450 mL) and MeOH (150 mL). Pd on carbon (10 wt. % loading, 2.48 g) was added. While stirring, the flask was evacuated and back-filled with hydrogen gas (repeated twice). The reactant was hydrogenated under hydrogen balloon for 3 hr. LC-MS showed the reaction was complete. The reaction mixture was filtered through a fritted funnel filled with a thin layer of celite. The solid in the funnel was washed with EtOAc/MeOH (2:1; total 900 mL). The filtrate was concentrated to afford a greasy white foam which was co-evaporated with DCM (3×100 mL). The product was then dried on high vacuum over two nights to afford 1-(((2R,3R,4R,5R,6R)-3-acetamido-4,5-diacetoxy-6-(acetoxymethyl)tetrahydro-2H-pyran-2-yl)oxy)-16,16-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,11,18-trioxo-14-oxa-6,10,17-triazanonacosan-29-oic acid as a white foam (29.5 g, 100% yield). ¹H NMR (400 MHz, DMSO-d₆) δ 11.96 (s, 1H), 7.87-7.79 (m, 6H), 7.74 (t, J=5.6 Hz, 3H), 6.99 (s, 1H), 5.21 (d, J=3.4 Hz, 3H), 4.96 (dd, J=11.2, 3.4 Hz, 3H), 4.48 (d, J=8.5 Hz, 3H), 4.06-3.98 (m, 9H), 3.87 (dt, J=11.2, 8.8 Hz, 3H), 3.70 (dt, J=10.5, 5.6 Hz, 3H), 3.58-3.48 (m, 12H), 3.40 (dt, J=9.6, 6.0 Hz, 3H), 3.03 (p, J=6.3 Hz, 12H), 2.27 (t, J=6.4 Hz, 6H), 2.18 (t, J=7.4 Hz, 2H), 2.10 (s, 9H), 2.07-2.01 (m, 8H), 1.99 (s, 9H), 1.89 (s, 9H), 1.77 (s, 9H), 1.56-1.41 (m, 22H), 1.28-1.15 (m, 12H); MS (ESI), 1004.00 [M/2+ H]⁺.

### Example 40. Synthesis of a C12 Linker Tri-Antennary GalNAc Phosphoramidite

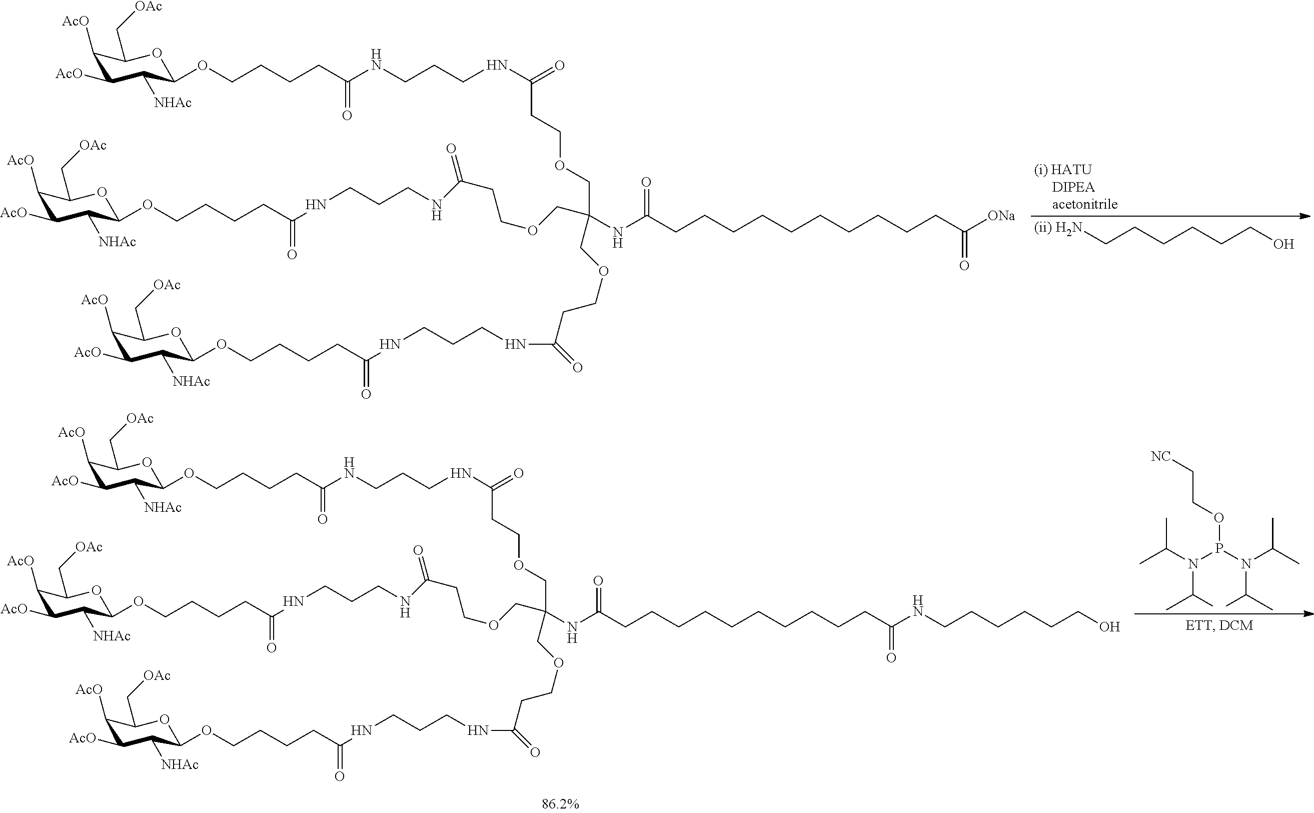

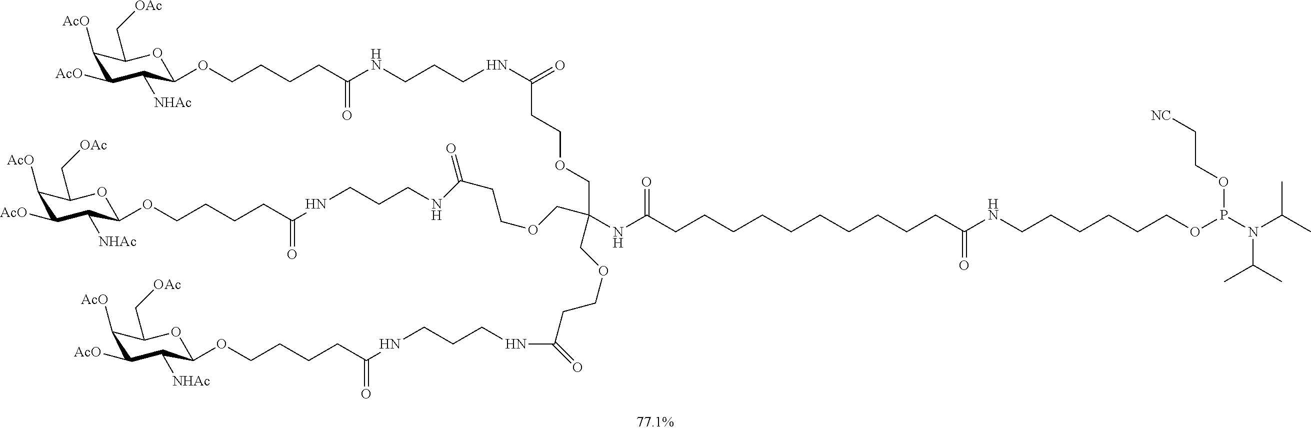

Step 1: To a round-bottom flask were charged with GalNAc acetyl derivative (20.0 g, 9.86 mmol), HATU (3.94 g, 10.35 mmol) and ACN (165 mL). While stirring, DIPEA (2.58 mL, 14.79 mmol) was added. Stirred for 10 min to give precursor A. 6-amino-1-hexanol (1.16 g, 9.86 mmol) was dissolved in ACN (65 mL) in another rbf. While stirring, precursor A was poured slowly to the amino alcohol solution in a period of 2 min. Additional DIPEA (0.5 mL) was added. The reaction solution was stirred for 45 min. LC-MS showed the reaction was complete. The volatiles were evaporated. The resulting crude product was dissolved in DCM (600 mL) and washed with water (2×50 mL). The organic layer was dried over sodium sulfate, filtered, and concentrated. The resulting crude residue was purified by normal phase column chromatography (ISCO 220 g gold cartridge) applying 5-25% MeOH in DCM as the gradient to afford [(3R,5S,6R)-5-acetamido-6-[5-[3-[3-[3-[3-[3-[5-[(2R,3S,5R)-3-acetamido-4,5-diacetoxy-6-(acetoxymethyl)tetrahydropyran-2-yl]oxypentanoylamino]propylamino]-3-oxo-propoxy]-2-[[3-[3-[5-[(2R,3S,5R)-3-acetamido-4,5-diacetoxy-6-(acetoxymethyl)tetrahydropyran-2-yl]oxypentanoylamino]propylamino]-3-oxo-propoxy]methyl]-2-[[12-(6-hydroxyhexylamino)-12-oxo-dodecanoyl]amino]propoxy]propanoylamino]propylamino]-5-oxo-pentoxy]-3,4-diacetoxy-tetrahydropyran-2-yl]methyl acetate as a solid (17.9 g, 86.2% yield). ¹H NMR (400 MHz, DMSO-d₆) δ 7.86-7.77 (m, 6H), 7.77-7.68 (m, 4H), 6.98 (s, 1H), 5.21 (d, J=3.4 Hz, 3H), 4.96 (dd, J=11.3, 3.4 Hz, 3H), 4.48 (d, J=8.5 Hz, 3H), 4.33 (t, J=5.1 Hz, 1H), 4.08-3.95 (m, 9H), 3.87 (dt, J=11.2, 8.8 Hz, 3H), 3.70 (dt, J=10.3, 5.6 Hz, 3H), 3.61-3.46 (m, 12H), 3.46-3.33 (m, 5H), 3.02 (h, J=6.1 Hz, 14H), 2.27 (t, J=6.4 Hz, 6H), 2.10 (s, 9H), 2.08-2.00 (m, 10H), 1.99 (s, 9H), 1.89 (s, 9H), 1.77 (s, 9H), 1.56-1.31 (m, 26H), 1.31-1.14 (m, 16H); MS (ESI), 1054.0 [M/2+H]⁺.

Step 2: The solution of dry [(3R,5S,6R)-5-acetamido-6-[5-[3-[3-[3-[3-[3-[5-[(2R,3S,5R)-3-acetamido-4,5-diacetoxy-6-(acetoxymethyl)tetrahydropyran-2-yl]oxypentanoylamino]propylamino]-3-oxo-propoxy]-2-[[3-[3-[5-[(2R,3S,5R)-3-acetamido-4,5-diacetoxy-6-(acetoxymethyl)tetrahydropyran-2-yl]oxypentanoylamino]propylamino]-3-oxo-propoxy]methyl]-2-[[12-(6-hydroxyhexylamino)-12-oxo-dodecanoyl]amino]propoxy]propanoylamino]propylamino]-5-oxo-pentoxy]-3,4-diacetoxy-tetrahydropyran-2-yl]methyl acetate (32.19 g, 15.29 mmol) in DCM (90 mL) was cooled to 0° C. 2-Cyanoethyl N,N,N′,N′-tetraisopropylphosphorodiamidite (9.71 mL, 30.58 mmol) was added dropwise in a period of 3 min. The solution was stirred for 5 min. 5-(Ethylthio)-1H-tetrazole (2.29 g, 17.58 mmol) was added in one portion. Continued to stir first at 0° C. for 5 min then at rt for 6 hr. TLC showed the reaction was complete. Triethylamine (8.3 mL) was added. Stirred for 5 min. The mixture was concentrated. The resulting crude product was purified by normal phase column chromatography applying 0-80% ACN in EtOAc (each mobile phase contained 5% triethylamine) as the gradient to afford the desired C12 linker tri-antennary GalNAc phosphoramidite as an off-white foam (27.2 g, 77.1% yield). ¹H NMR (600 MHz, DMSO-d₆) δ 7.86-7.78 (m, 6H), 7.73 (t, J=5.7 Hz, 3H), 7.69 (t, J=5.6 Hz, 1H), 6.98 (s, 1H), 5.21 (d, J=3.4 Hz, 3H), 4.96 (dd, J=11.2, 3.5 Hz, 3H), 4.48 (d, J=8.5 Hz, 3H), 4.06-3.98 (m, 9H), 3.87 (dt, J=11.2, 8.8 Hz, 3H), 3.74-3.67 (m, 5H), 3.62-3.50 (m, 16H), 3.40 (dt, J=9.7, 6.2 Hz, 3H), 3.02 (dq, J=15.4, 8.7, 7.7 Hz, 14H), 2.75 (t, J=5.9 Hz, 2H), 2.27 (t, J=6.4 Hz, 6H), 2.10 (s, 9H), 2.07-2.00 (m, 1OH), 1.99 (s, 9H), 1.89 (s, 9H), 1.77 (s, 9H), 1.56-1.40 (m, 26H), 1.39-1.17 (m, 16H), 1.13 (t, J=6.6 Hz, 12H); ³¹P NMR (243 MHz, DMSO-d₆) δ 146.32; MS (ESI), 1153.24 [M/2+H]⁺.

### Example 41. Preparation of Oligonucleotide Compositions

Various technologies for preparing oligonucleotides and oligonucleotide compositions (both stereorandom and chirally controlled) are known and can be utilized in accordance with the present disclosure, including, for example, methods and reagents described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, and/or WO 2020/191252, the methods and reagents of each of which are incorporated herein by reference.

A number of oligonucleotide compositions were synthesized and assessed. Observed MS data of oligonucleotides in some recently prepared oligonucleotide compositions are as follows (when multiple numbers are presented for the same oligonucleotide, the numbers can be MS data observed in different batches/experiments): WV-20666: 10167.1; WV-20689: 10183; WV-20690: 10198.4; WV-20691: 10215.3; WV-20692: 10230.3; WV-20693: 10246.5; WV-20694: 10262.7; WV-20695: 10278.9; WV-20696: 10294.3; WV-20697: 10311.3; WV-20698: 10327; WV-20699: 10342.9; WV-20700: 10358.5; WV-20701: 10376; WV-20702: 10391.1; WV-20703: 10407.5; WV-20704: 10423.6; WV-20706: 10199; WV-20707: 10215.3; WV-20708: 10230.6; WV-20709: 10246.5; WV-20710: 10262.6; WV-20711: 10279.3; WV-20712: 10294.2; WV-20713: 10310.8; WV-20714: 10327; WV-20715: 10342.9; WV-20716: 10358.7; WV-20717: 10246.3; WV-20718: 10262.7; WV-20719: 10278.3; WV-20720: 10294.2; WV-20721: 10311.4; WV-20722: 10327.1; WV-20723: 10342.8; WV-20724: 10358.7; WV-20725: 10374.8; WV-20726: 10391; WV-20727: 10182.9; WV-20728: 10182.7; WV-20729: 10182.7; WV-20730: 10182.9; WV-20731: 10230.8; WV-20732: 10199.1; WV-20733: 10663.7; WV-20734: 10194.7; WV-20735: 10222.7; WV-20736: 10250.5; WV-20737: 10278.3; WV-20738: 10306.7; WV-20739: 10334.8; WV-20740: 10362.9; WV-20741: 10194.8; WV-20742: 10208.5; WV-20743: 10236.8; WV-20744: 10263.9; WV-20745: 10293.1; WV-20746: 10320.4; WV-20747: 10093.9; WV-20748: 10098.1; WV-20749: 10101.9; WV-20750: 10106.4; WV-20751: 10110.5; WV-20752: 10113.5; WV-20753: 10118.3; WV-20754: 10122.6; WV-20755: 10098; WV-20756: 10100; WV-20757: 10104.3; WV-20758: 10107.7; WV-20759: 10111.8; WV-20760: 10116.7; WV-23388: 10098; WV-23395: 10612.3; WV-24111: 10046.8; WV-24112: 10047; WV-24113: 10047; WV-24114: 10046.8; WV-24115: 10047; WV-24116: 10046.8; WV-24117: 10046.9; WV-24118: 10046.8; WV-24119: 10046.9; WV-24120: 10047.1; WV-24121: 10047; WV-24122: 10047.1; WV-24123: 10047; WV-24124: 10047; WV-24125: 10046.9; WV-24126: 10046.9; WV-24127: 10047; WV-24128: 10046.5; WV-24129: 10047; WV-24130: 10046.8; WV-24131: 10046.8; WV-24132: 10047; WV-24133: 10047.1; WV-24134: 10047; WV-24135: 10047; WV-24136: 10046.9; WV-24137: 10047.1; WV-24138: 10047; WV-24139: 10046.8; WV-24140: 10046.4; WV-24141: 10046.9; WV-24142: 10047; WV-24143: 10047.1; WV-24144: 10047; WV-24145: 10047.1; WV-24146: 10046.9; WV-24147: 10046.7; WV-24148: 10047; WV-24149: 10047; WV-24150: 10047.1; WV-24151: 10047.1; WV-24152: 10047.1; WV-24153: 10047.1; WV-24154: 10047.1; WV-24155: 10047.1; WV-24156: 10046.7; WV-24157: 10047; WV-24158: 10047.1; WV-27457: 12613.1; WV-27458: 11954.6; WV-27459: 12631; WV-27460: 11972.7; WV-27521: 10064.1; WV-31133: 10737.8; WV-31134: 10869.1; WV-31135: 10790.3; WV-31137: 10779.4; WV-31138: 10788.2; WV-31139: 10039.1; WV-31140: 10168.8; WV-31141: 10091.0; WV-31143: 10079.0; WV-31144: 10089.6; WV-31632: 10772.7; WV-31633: 10786.6; WV-31634: 10072.7; WV-31635: 10087.2; WV-31748: 10762.5; WV-31749: 10064.4; WV-28788: 10169.1; WV-27458: 11954.6; WV-31940: 10285.5; WV-35741: 12352.0.

As described and confirmed herein, technologies of the present disclosure are useful for preparing various compositions of oligonucleotides comprising various structural features. In some embodiments, as confirmed herein, provided technologies, e.g., those utilizing chiral auxiliaries comprising electron-withdrawing groups (e.g., R_{C11} comprising electron-withdrawing groups (e.g., —SO₂R^{C1}—C(O)R^{C1}, etc.)) are particularly useful for preparing chirally controlled compositions of oligonucleotides comprising 2′-OH sugars (e.g., sugars with R^{2s}═OH, such as sugars typically found in natural RNA), particularly when such sugars are bonded to chirally controlled internucleotidic linkages. A preparation of WV-29874 is described below as an example.

An automated solid-phase synthesis of a chirally controlled oligonucleotide composition (WV-29874) at 25 umol scale was performed according to the cycles below:

| | | | | waiting |
|---|---|---|---|---|
| step | operation | reagents and solvent | volume | time |
| 1 | detritylation | 3% TCA / DCM | 10 mL | 65 s |
| 2 | coupling | 0.2M monomer / 20% | 0.5 mL | 8 min |
| | | IBN-MeCN 0.5M | 1.0 mL | |
| | | PhIMT / MeCN | | |
| 3 | cap-1 | 20% Ac₂O, 30% 2,6-lutidine / | 2.0 mL | 2 min |
| | | MeCN | | |
| 4 | sulfurization | 0.2M XH / pyridine | 2.0 mL | 6 min |
| 5 | cap-2 | 20% Ac₂O, 30% 2,6-lutidine / | 1.0 mL | 45 s |
| | | MeCN 20% MeIm / MeCN | 1.0 mL | |

IBN: isobutyronitrile; MeIm: N-methylimidazole; PhIMT: N-phenylimidazolium triflate; XH: xanthane hydride. The cycles were performed multiple times until the desired length was achieved. PSM phosphoramidites were utilized for formation of chirally controlled internucleotidic linkages (for 2′-OH, protected with TBS (t-butyldimethylsilyl)).

After completion of the synthesis cycles, PSM chiral auxiliary groups were removed by an anhydrous base treatment (DEA treatment). The CPG was treated with 40% MeNH₂ (5.0 mL) for 30 min at 35° C., then cooled to room temperature and the CPG was separated by membrane filtration, washed with 8.0 mL of DMSO. To the filtrate, TEA (triethylamine)-3HF (5.0 mL) was added and stirred for 1 h at 45° C. which can remove TBS protection groups from 2′-OH. The reaction mixture was cooled to room temperature and diluted with 10 mL of 50 mM NaOAc (pH 5.2). The crude material was analyzed by LTQ and RP-UPLC. The crude materials were purified by RP-HPLC with a linear gradient of MeCN in 50 mM TEAA (triethylammonium acetate), desalting by tC18 SepPak cartridge to obtain the target oligonucleotide.

Desalting was performed using the following procedure:

Evaporate MeCN from samples if present.
Condition column with 4 CV of 100% acetonitrile (HPLC grade).
Rinse column with 2 CV of 40% MeCN in Millipore Bio-Pak water, Endotoxin-Free.
Rinse column with 4 CV of water (Millipore Bio-Pak, Endotoxin-Free).
Equilibrate column with 2 CV of 50 mM TEAA in Millipore Bio-Pak water, Endotoxin-Free.
Load pure fractions onto equilibrated column. In some embodiments, loading by gravity provide the greatest amount of binding, loading slowly with vacuum provide decent binding, and loading quickly with vacuum result in poor binding.
Wash column with 2 CV of BioPak water to wash away TEAA.
Wash column with 2 CV of 100 mM NaOAc to exchange the ammonium on the backbone of oligonucleotides with Sodium instead.
Wash column with BioPak water until conductivity of eluent is <20 uS/cm.
Elute product with 2 column volumes of 40% MeCN in Millipore Bio-Pak water, Endotoxin-Free.
Place on Speed-vac overnight at 30° C. to remove acetonitrile and to concentrated.

Results from one preparation: Synthesis scale: 25 umol; Crude ODs: 874 ODs; Crude UPLC purity: 32.17%; Crude LTQ purity: 62.45%; Final ODs: 59.8 OD; Final UPLC purity:59.85%; Final MS purity: 74.51%; and Final Observed MS: 10064.4 (Calculated 10,063.68).

### Example 42. Provided Technologies Comprising Chirally Controlled Oligonucleotide Compositions can Provide High Activities Compared to Stereorandom Oligonucleotide Compositions

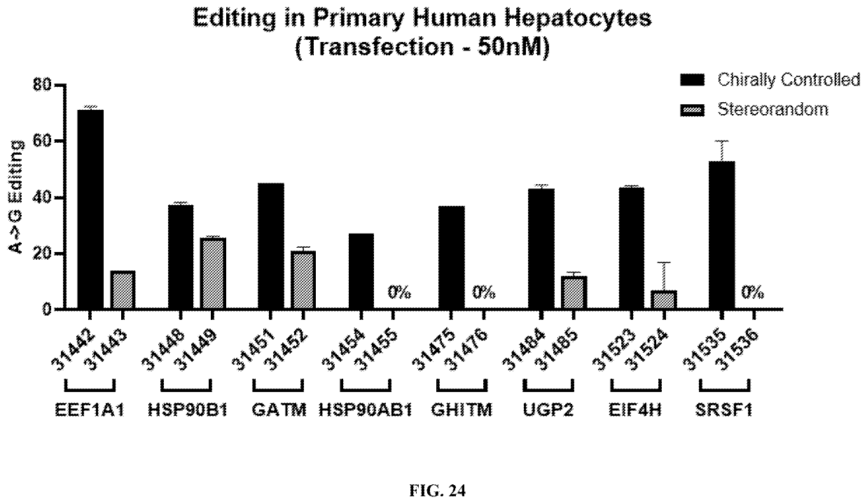

Among other things, the present disclosure demonstrates that provided compositions comprising various chirally controlled internucleotidic linkages can provide high activities compared to stereorandom compositions comprising no chirally controlled internucleotidic linkages. For example, as illustrated in FIG. 24, various chirally controlled oligonucleotide compositions were assessed and provided higher activities compared to stereorandom compositions for various target transcripts. Primary human hepatocytes were transfected with 50 nM oligonucleotides. RNA was harvested 48 hours later and percentage of edited transcripts was quantified by Sanger sequencing (n=2 biological replicates).

### Example 43. Provided Technologies Comprising Oligonucleotides with Modified Internucleotidic Linkages can Provide High Activities

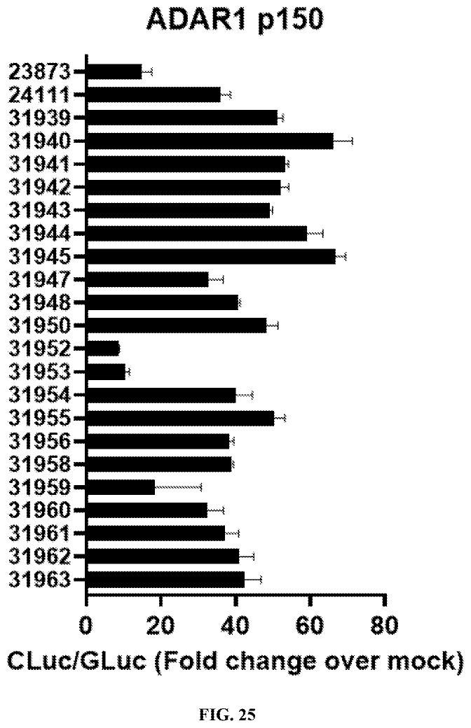

Among other things, the present disclosure demonstrates that provided technologies comprising various internucleotidic linkage types and patterns can provide editing activities, for example, as illustrated in FIG. 25, in which various oligonucleotide compositions were assessed. Compositions target a premature UAG stop codon within the cLuc coding sequence. 293T cells were transfected with ADAR1-p150, luciferase reporter construct, and indicated compositions at 3.3 nM oligonucleotide concentrations. cLuc activity was normalized to Glue expression in mock treated samples (n=2 biological replicates). In some embodiments, incorporation of non-negatively charged internucleotidic linkages such as n001 at certain positions can provide higher editing activities.

### Example 44. Provided Technologies Comprising Oligonucleotides Comprising Additional Chemical Moieties can Provide High Activities

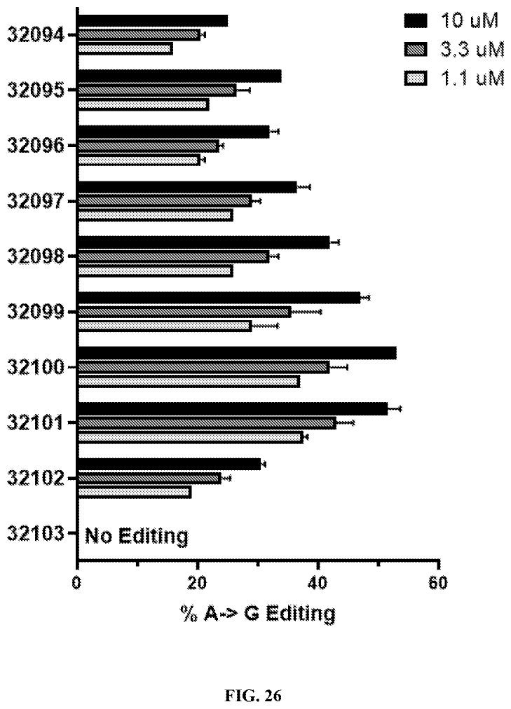

Among other things, the present disclosure demonstrates that provided technologies comprising various internucleotidic linkage patterns, sugar modification patterns, and/or additional chemical moieties can provide high editing activities with or without exogenous ADAR, for example, as illustrated in FIG. 26, in which various oligonucleotide compositions were assessed. Primary monkey hepatocytes were treated gymnotically with indicated oligonucleotide compositions. Editing of target was measured by Sanger sequencing (n=2 biological replicates). In some embodiments, certain structural elements, e.g., 2′-F modified sugars in second subdomains, Rp phosphorothioate linkages bonded to second subdomain nucleosides (as shown, two Rp phosphorothioate linkages bonded to a certain second subdomain nucleoside), and/or non-negatively charged internucleotidic linkages such as n001 at certain locations can improve editing efficiency.

### Example 45. Provided Technologies Comprising Oligonucleotides with Modified Internucleotidic Linkages, Sugar Modifications, and/or Additional Chemical Moieties can Provide High Activities

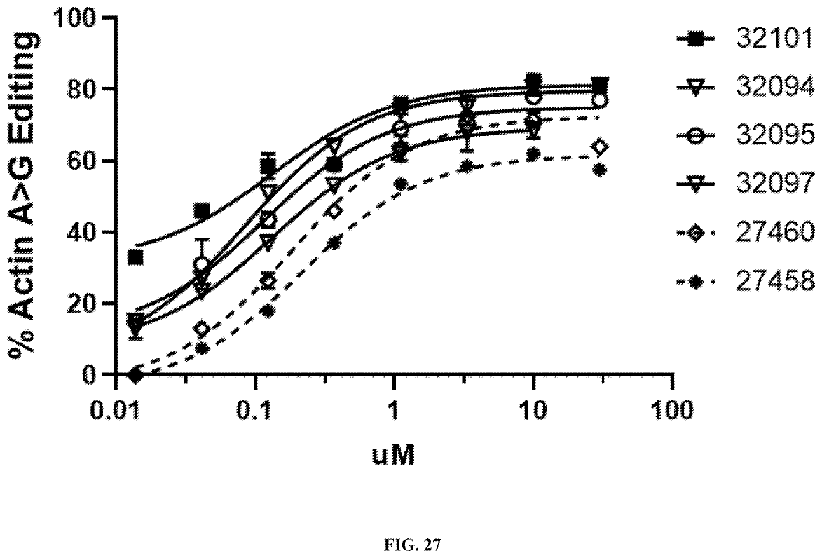

Among other things, the present disclosure demonstrates that provided technologies comprising oligonucleotides with modified internucleotidic linkages, sugar modifications, and/or additional chemical moieties can provide high activities, for example, as illustrated in FIG. 27, in which various oligonucleotide compositions were assessed. Primary human hepatocytes were treated gymnotically with indicated oligonucleotide compositions. Percentage of edited transcripts was quantified by Sanger sequencing (n=2 biological replicates). In some embodiments, certain structural elements, e.g., 2′-F modified sugars in second subdomains, Rp phosphorothioate linkages bonded to second subdomain nucleosides, positioning of mismatches, and/or non-negatively charged internucleotidic linkages such as n001 at certain locations can improve editing efficiency.

### Example 46. Provided Technologies Comprising Oligonucleotides Comprising Various Modified Internucleotidic Linkages, Sugar Modifications and/or Additional Moieties can Provide High Activities

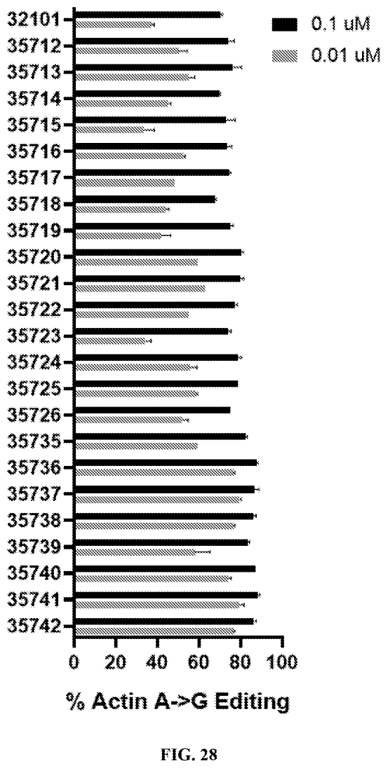

Among other things, the present disclosure demonstrates that provided technologies comprising various internucleotidic linkage modifications, sugar modifications and/or additional moieties and patterns thereof can provide high editing activities with or without exogenous ADAR, for example, as illustrated in FIG. 28, in which various oligonucleotide compositions were assessed. Primary human hepatocytes were treated gymnotically with indicated oligonucleotide compositions. Percentage of edited transcripts was quantified by Sanger sequencing (n=2 biological replicates). In some embodiments, certain structural elements, e.g., 2′-F modified sugars in second subdomains, Rp phosphorothioate linkages bonded to second subdomain nucleosides, positioning and/or presence or absence of mismatches, and/or non-negatively charged internucleotidic linkages such as n001 at certain locations can improve editing efficiency. Example 47. Provided technologies comprising oligonucleotides with modified internucleotidic linkages, sugar modifications, and/or additional chemical moieties can provide high activities.

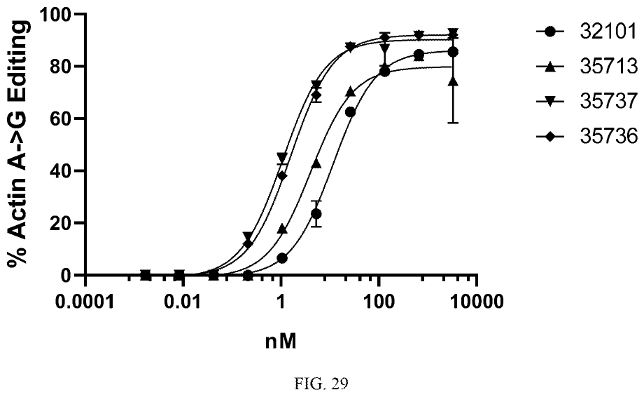

Among other things, the present disclosure demonstrates that provided technologies comprising oligonucleotides with modified internucleotidic linkages, sugar modifications, and/or additional chemical moieties can provide high activities, for example, as illustrated in FIG. 29 in which various oligonucleotide compositions were assessed (as in various Figures and other places, detailed descriptions of oligonucleotides/compositions can be found in, e.g., Table 1 (e.g., 1A, 1B, 1C and/or 1D); in some cases, oligonucleotides/compositions may be referred to by numbers only (e.g., in FIG. 29, WV-32101, WV-35713, WV-35737 and WV-35736 are referred to by their numbers only (32101, 35713, 35737 and 35736, respectively))). Primary human hepatocytes were treated gymnotically with indicated oligonucleotide compositions at indicated concentrations. ADAR was endogenous. Compositions target an adenosine in the 3′ UTR of beta-actin mRNA. Percentage of edited transcripts was quantified by Sanger sequencing (n=2 biological replicates). In some embodiments, certain patterns of non-negatively charged internucleotidic linkages and/or increased numbers of non-negatively charged internucleotidic linkages can increase editing efficiency. In some embodiments, reducing mismatches, and/or utilizing nucleosides comprising 2′-F modified sugars at second domains (e.g., second subdomains (e.g., next to a nucleoside (e.g., at the 5′-side) opposite to a target adenosine to be edited)) can provide increased editing efficiency.

### Example 47. Provided Technologies Comprising Oligonucleotides with Modified Internucleotidic Linkages, Sugar Modifications, and/or Additional Chemical Moieties can Provide High Activities

Among other things, the present disclosure demonstrates that provided technologies comprising oligonucleotides with modified internucleotidic linkages, sugar modifications, and/or additional chemical moieties can provide high activities, for example, as illustrated in FIG. 29, in which various oligonucleotide compositions were assessed. Primary human hepatocytes were treated gymnotically with indicated oligonucleotide compositions at indicated concentrations. ADAR was endogenous. Compositions target an adenosine in the 3′ UTR of beta-actin mRNA. Percentage of edited transcripts was quantified by Sanger sequencing (n=2 biological replicates). In some embodiments, certain patterns ofnon-negatively charged internucleotidic linkages and/or increased numbers of non-negatively charged internucleotidic linkages can increase editing efficiency. In some embodiments, reducing mismatches, and/or utilizing nucleosides comprising 2′-F modified sugars at second domains (e.g., second subdomains (e.g., next to a nucleoside (e.g., at the 5′-side) opposite to a target adenosine to be edited)) can provide increased editing efficiency.

### Example 48. Provided Technologies Comprising Oligonucleotides with Modified Internucleotidic Linkages, Sugar Modifications, and/or Additional Chemical Moieties can Provide High Activities

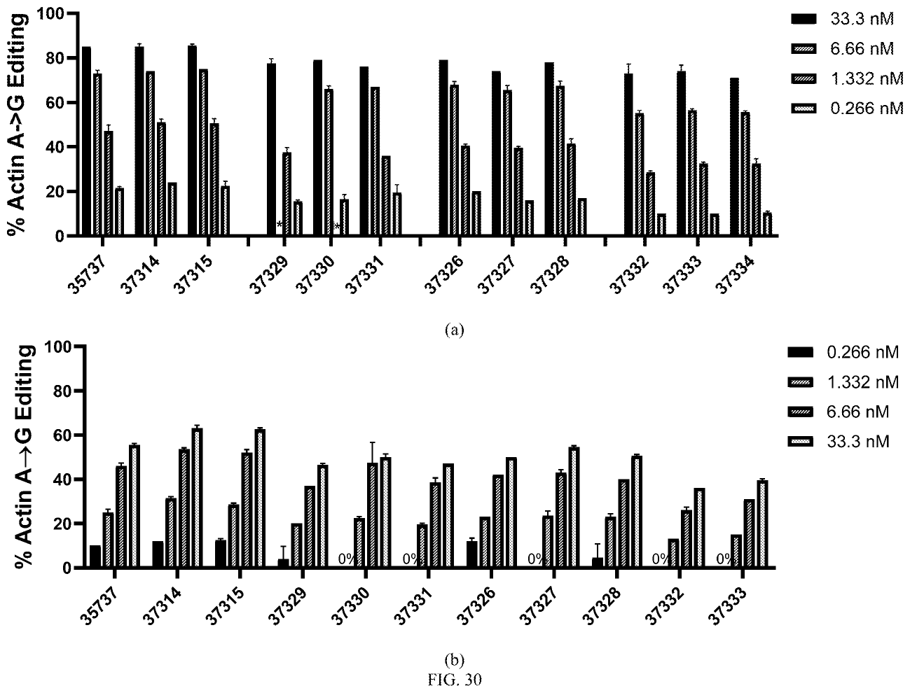

Among other things, the present disclosure demonstrates that provided technologies comprising oligonucleotides with modified internucleotidic linkages, sugar modifications, and/or additional chemical moieties can provide high activities, for example, as illustrated in FIG. 30, in which various oligonucleotide compositions were assessed. Primary human or monkey hepatocytes were treated gymnotically with indicated oligonucleotide compositions at indicated concentrations. ADAR was endogenous. Compositions target an adenosine in the 3′ UTR of beta-actin mRNA. Percentage of edited transcripts was quantified by Sanger sequencing (n=2 biological replicates). As shown in FIG. 30 sugar modifications other than 2′-F, e.g., 2′-OR where R is optionally substituted C₁₋₆ aliphatic (such as 2′-OMe and 2′-MOE), can be utilized at certain levels (e.g., numbers and/or percentages) and/or patterns in oligonucleotides including in first domains. Further, various patterns of backbone chiral centers, including patterns comprising stereochemistry of various non-negatively charged internucleotidic linkages, can be utilized for editing.

### Example 49. Chiral Control can Improve Editing Efficiency

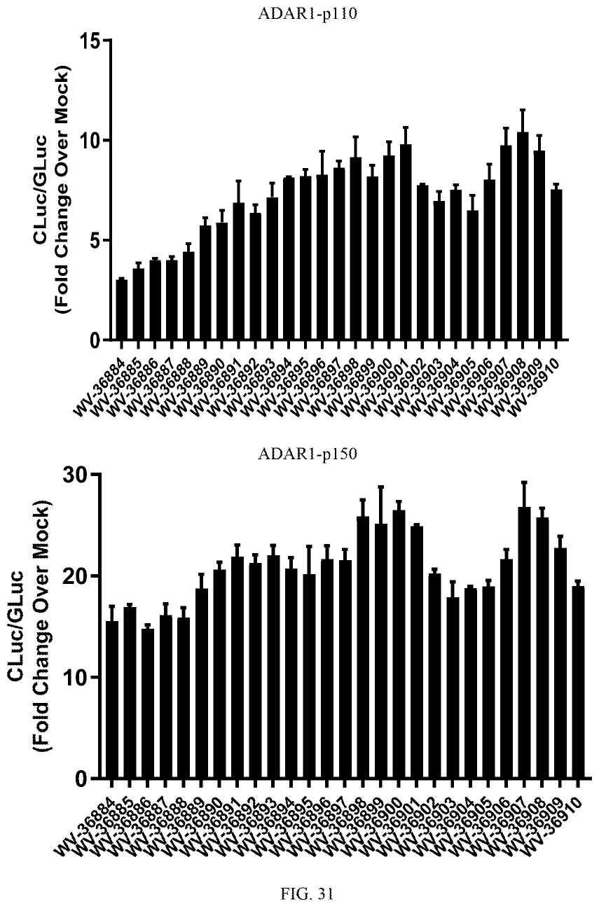

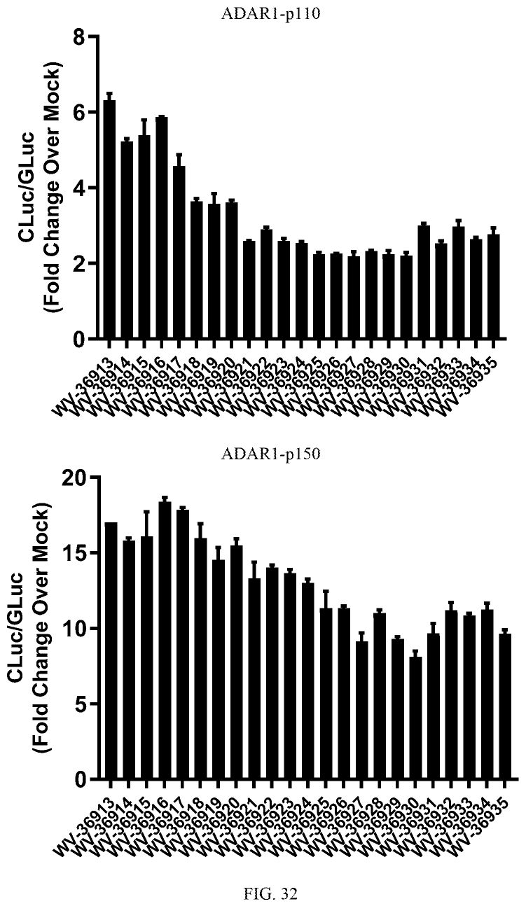

Among other things, the present disclosure demonstrates that chiral control of modified internucleotidic linkages such as phosphorothioate internucleotidic linkages can improve activities. In some embodiments, as illustrated in FIG. 31 and FIG. 32, increased numbers/levels of chirally controlled internucleotidic linkages (e.g., chirally controlled Sp phosphorothioate internucleotidic linkages) in first domains and/or second domains (and subdomains thereof) and/or oligonucleotides can provided improved editing efficiency. In some embodiments, one or more non-chirally controlled internucleotidic linkages can be utilized at certain positions to provide compositions of certain levels of activities. Compositions all target a premature UAG stop codon within the cLuc coding sequence. 293T cells were transfected with plasmids encoding ADAR1-p110 or -p150, luciferase reporter construct and indicated oligonucleotide compositions. cLuc activity was normalized to Glue expression in mock treated samples (n=2 biological replicates). In some embodiments, Sp phosphorothioate internucleotidic linkages increase RBD binding and/or deaminase domain activities of ADAR1-p110 and/or ADAR1-p150.

### Example 50. Chiral Control and Modified Internucleotidic Linkages can Improve Editing Efficiency

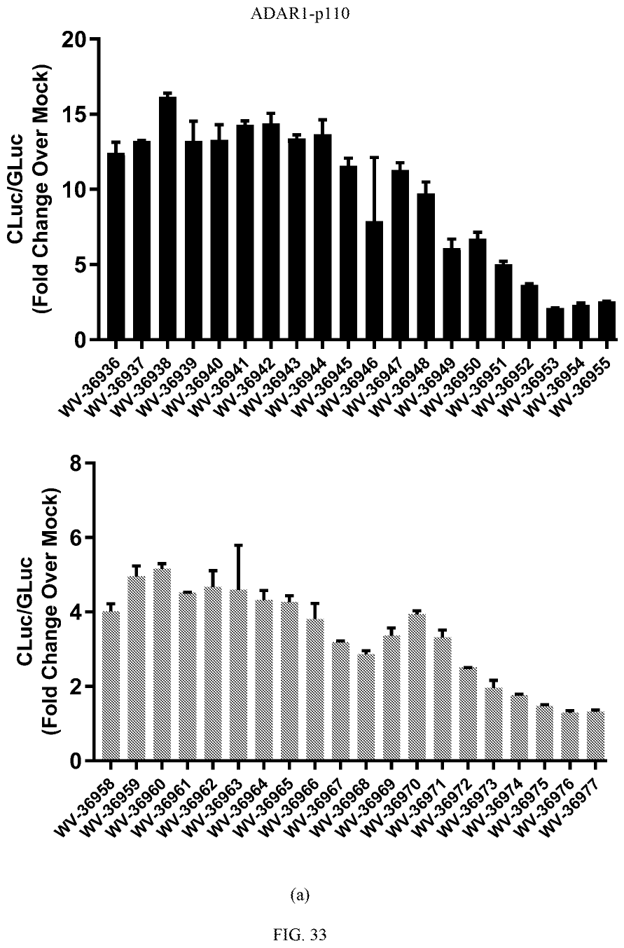

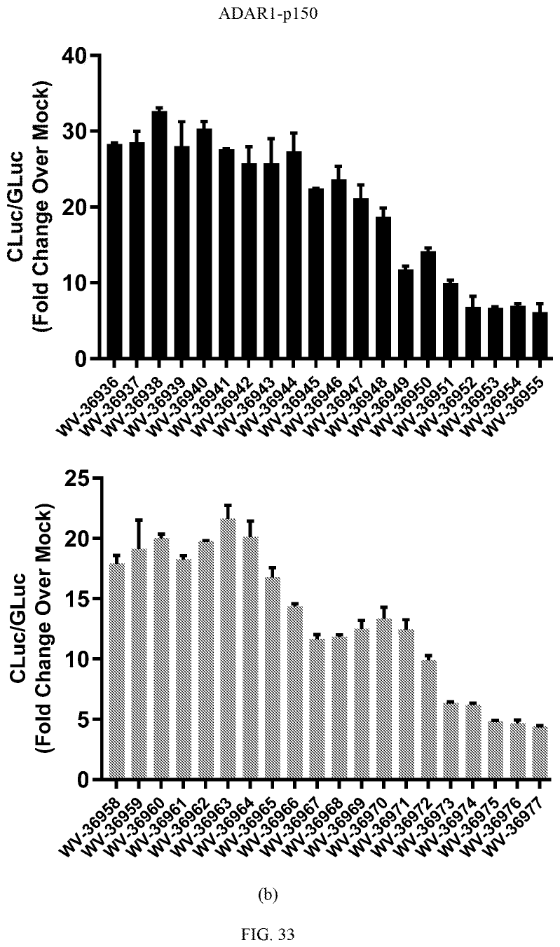

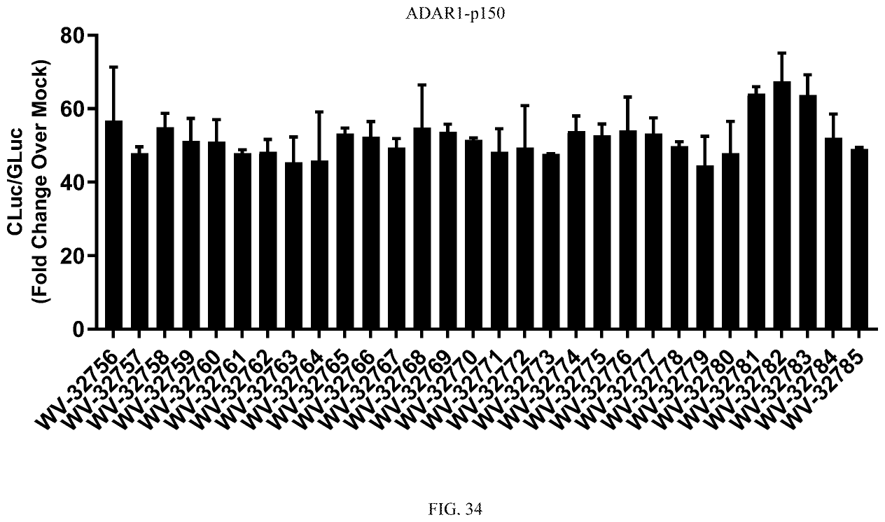

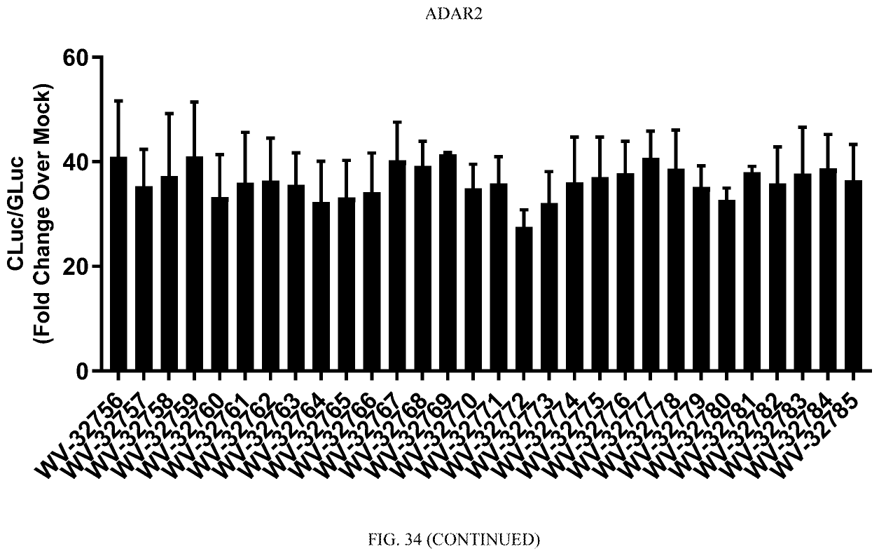

In some embodiments, the present disclosure demonstrates that compositions of oligonucleotides comprising modified internucleotidic linkages (e.g., phosphorothioate internucleotidic linkages) can provide improved editing activities. In some embodiments, the present disclosure provides compositions of oligonucleotides comprising chirally controlled modified internucleotidic linkages, and demonstrates that chiral control can improve desired activities. In some embodiments, as illustrated in FIG. 33, increased numbers/levels of modified internucleotidic linkages (e.g., phosphorothioate internucleotidic linkages) and/or chirally controlled internucleotidic linkages (e.g., chirally controlled Sp phosphorothioate internucleotidic linkages) in first domains and/or second domains (and subdomains thereof) and/or oligonucleotides can provided improved editing efficiency. In some embodiments, natural phosphate linkages can be utilized at certain positions to provide compositions of certain levels of activities (e.g., see FIG. 33 and FIG. 34).

### Example 51. Various Sugar Modifications May be Utilized to Provide Oligonucleotide Compositions with Desired Activities

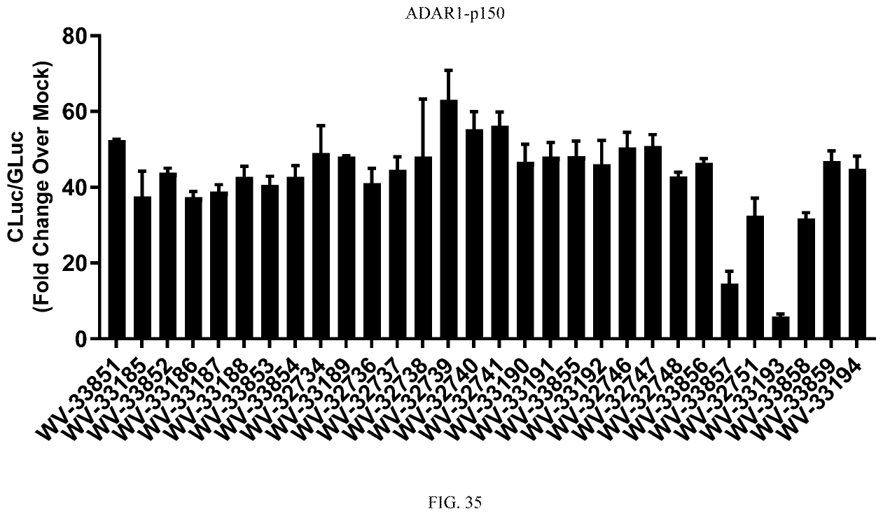

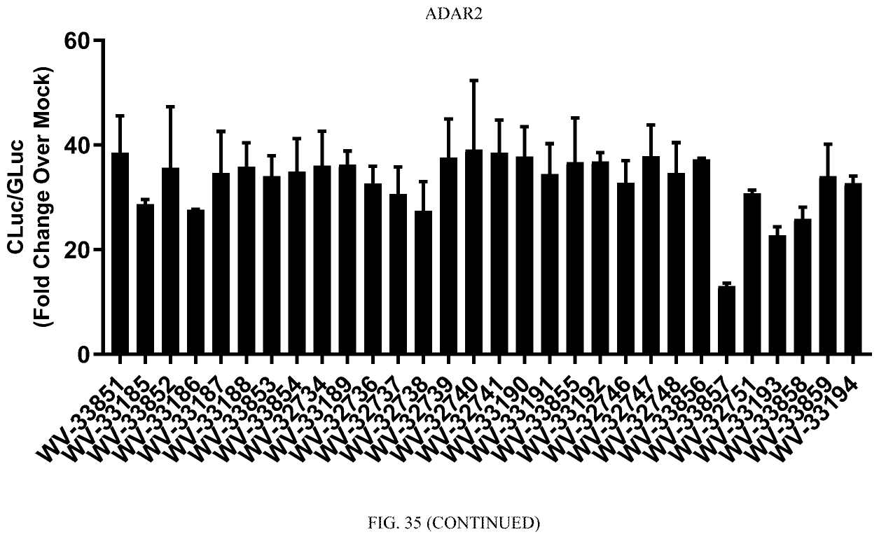

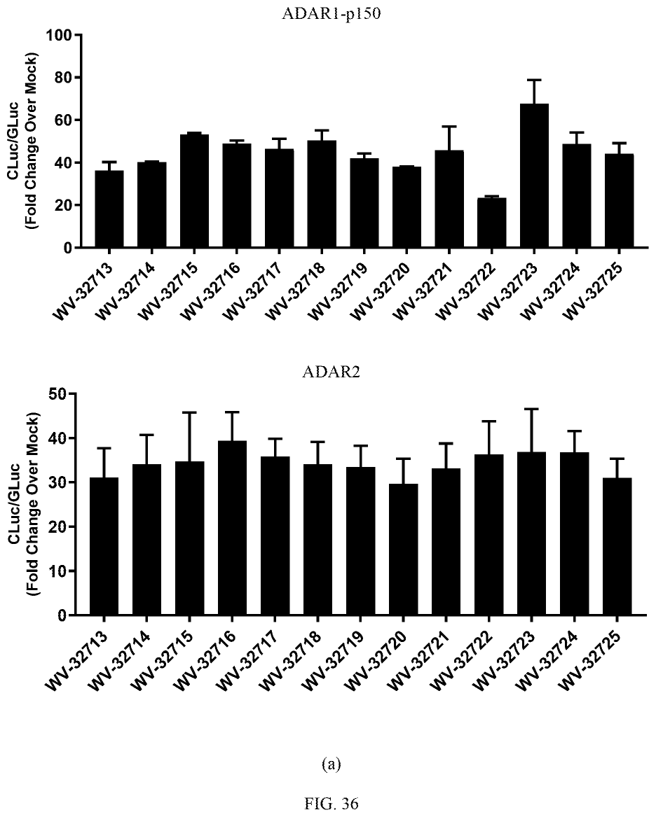

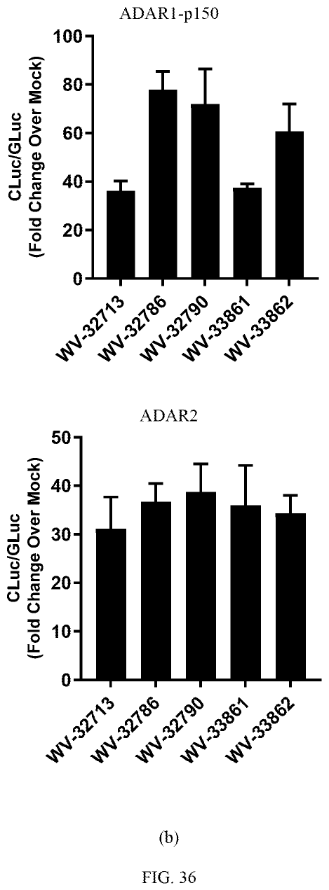

Among other things, the present disclosure demonstrates that various sugar modifications may be utilized in accordance with the present disclosure to provide oligonucleotides and compositions thereof that can provide editing activities. For example, 2′-modifications (e.g., 2′-OR wherein R is not hydrogen) may be utilized in oligonucleotides at various levels and/or patterns in accordance with the present disclosure to provide, e.g., adenosine editing activities. As shown in FIG. 35, 2′-OR modifications (wherein R is not —H) can be utilized in various positions. It is noted that, in some embodiments, it may be preferable to have sugar modifications other than 2′-MOE (e.g., to have 2′-OMe) at certain positions in certain contexts. As shown in FIG. 36, 2′-F modifications can be utilized in various positions. It is noted that, in some embodiments, it may be preferable to have sugar modifications other than 2′-F (e.g., to have 2′-OMe) at certain positions in certain contexts.

### Example 52. Provided Technologies can Provide High Levels of Activities In Vivo

As described above and confirmed herein, provided technologies can, among other things, provide high levels of editing activities in vivo, including in primates. As demonstrated herein, various provided technologies can provide high efficiency of adenosine editing is non-human primate models.

In some embodiments, non-naïve cynomolgus macaques (5-8 kg) at time of dosing were administered 5 mg/kg subcutaneous (SC) dose of WV-37314, WV-37315, or WV-37330 for 5 consecutive days. 48 hours post last dose, the animals where administered anesthesia and analgesia for biopsy procedures. Under anesthesia, liver biopsy samples were collected from all available animals. For each animal, two liver biopsy samples (80 to 120 mg) were collected, immediately weighed and placed into appropriate collection vials, and flash frozen in liquid nitrogen. Each individual sample was placed into a separate tube. Samples were then stored on dry ice until transferred to a freezer set to maintain−60 to−80° C.

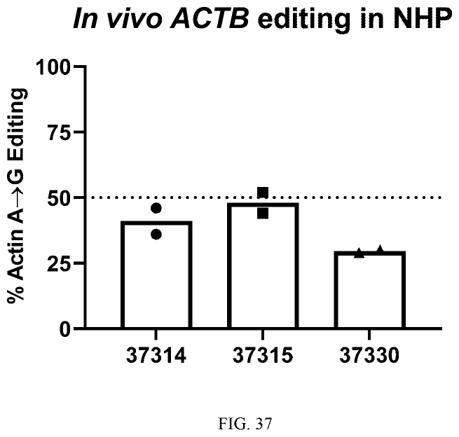

For RNA isolation, frozen tissue was added to 1000 uL of Trizol and homoogenized. 200 uL chloroform was added to each sample, shaken vigorously, incubated for 5 minutes, and then centrifuged at 10,000×g for 5 minutes. Supernatant (aqueous phase) was transferred to the binding plate from SV96 total RNA extraction kit (Promega) and RNA was extracted per protocol. cDNA was synthesized by adding 9 uL of total RNA to a 20 uL RT reaction using High Capacity cDNA Reverse Transcription Kit as recommended by the manufacturer (Applied Biosystems). 2 uL of cDNA was used in a PCR reaction with Phusion High-fidelity DNA polymerase (ThermoFisher Scientific: Catalog number #F530) to amplify the ACTB transcript using custom primers from IDT. PCR product was purified using AMPure XP magnetic beads per manufacturers protocol, and analyzed by Sanger sequencing (Genewiz, USA). Percent editing was then quantified using the EditR program (https://moriaritylab.shinyapps.io/editr_v10/). Certain results were presented in FIG. 37. As demonstrated, provided technologies provide high levels of editing activities in these primate animals.

### Example 53. Provided Technologies can Provide Long-Lasting Editing Activities In Vivo

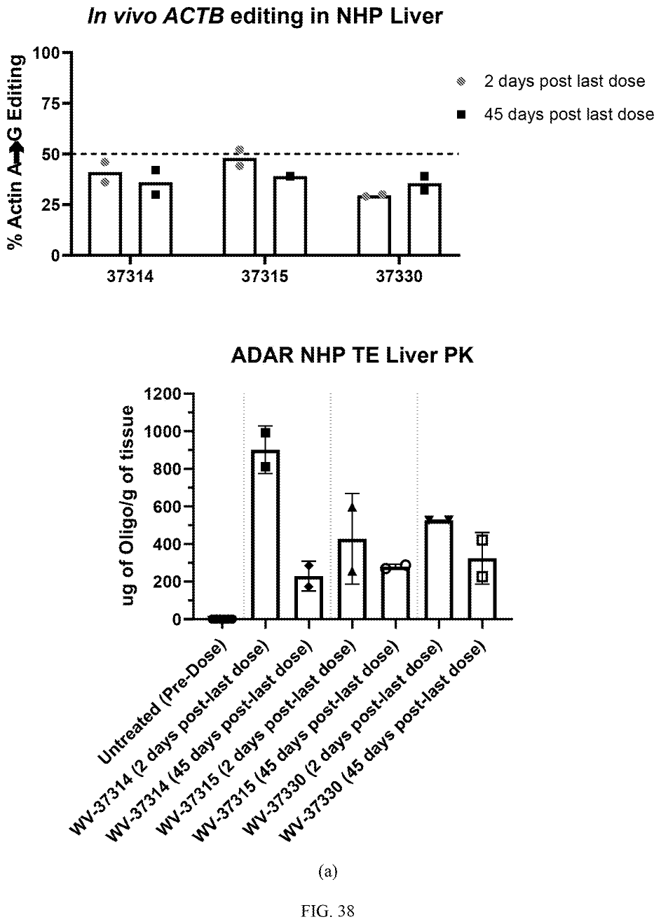

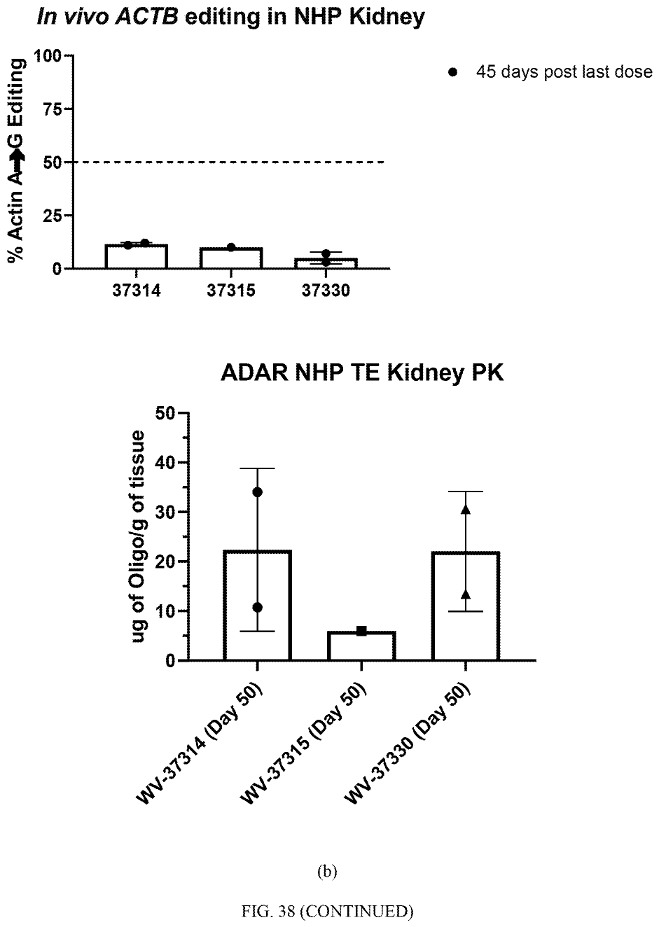

In some embodiments, the present disclosure provides oligonucleotide compositions that can, among other things, provide editing activities in vivo. As described, technologies of the provided technology can provide increased stability, high levels of editing, etc., as confirmed in this Example and FIG. 38. As demonstrated, provided technologies can provide desired editing activities for a long period of time, e.g., about or at least about 2, 3, 4, 5, 6, 7, 8, 9, 10, 15, 20, 25, 30, 35, 40, 45 or more days, after a last dose. In some embodiments, desired editing activities/levels of editing may be maintained for a long period of time, e.g., about or at least about 2, 3, 4, 5, 6, 7, 8, 9, 10, 15, 20, 25, 30, 35, 40, 45 or more days, after a last dose.

Certain data are presented in FIG. 38, which depict editing in non-human-primates (NHP) liver (a) and kidney (b) (non-naïve cynomolgus macaques (5-8 kg) at time of dosing). Compositions all target an adenosine in the 3′UTR of beta-actin mRNA. Animals were dosed with oligonucleotide compositions (WV-37314, WV-37315, and WV-37330) and liver biopsies were collected at Day 2 and 45 post last dose, and kidneys were collected at day 45 post last dose. As confirmed in FIG. 38, (a), all three oligonucleotide compositions (WV-37314, WV-37315, and WV-37330) provided editing ofACTB mRNA in vivo (range of 25-50% editing), without exogenous ADAR, in liver at both day 2 and at least 45 post last dose, and levels of editing remained high at least 45 days post last dose. Furthermore, as can be seen in FIG. 38, (b), all three oligonucleotide compositions (WV-37314, WV-37315, and WV-37330) provided editing of ACTB mRNA in vivo (range of 5-15% editing), without exogenous ADAR, in the kidney at least 45 days post last dose. Analysis was performed in both liver and kidney to assess oligonucleotide amounts. FIG. 38 illustrated certain data for liver at 2 and 45 days post last dose with a range of 150-1000 ug/g of tissue, and for kidney 45 days post last dose with a range of 5-35 ug/g of tissue, confirming delivery and/or stability of provided oligonucleotide compositions to and in various tissues. As confirmed in FIG. 38, in some embodiments, provided oligonucleotides may be selectively delivered to and/or maintained at higher levels in one or more tissues (e.g., liver) over others (e.g., kidney).

### Example 54. Provided Technologies can Provide Editing Activities in Various Systems

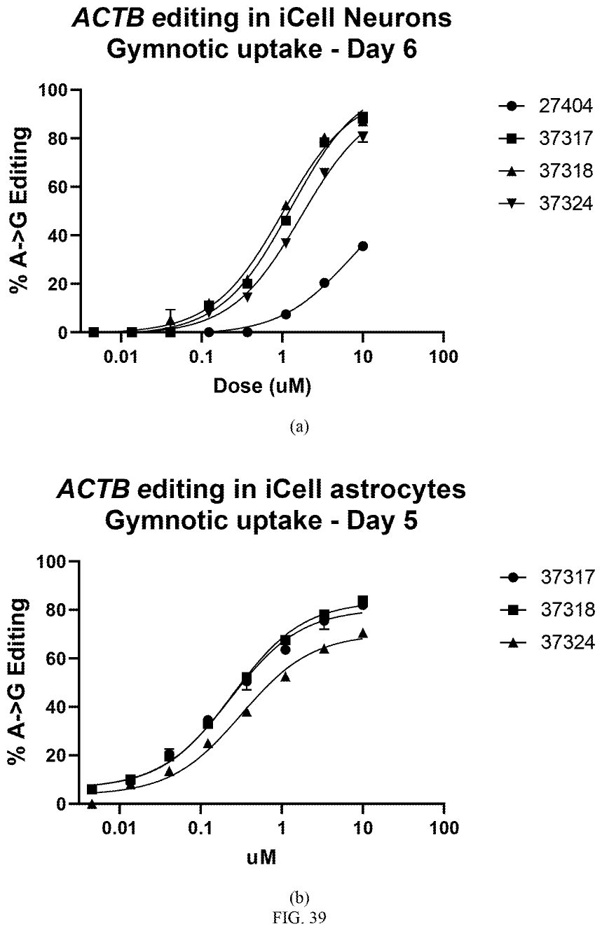

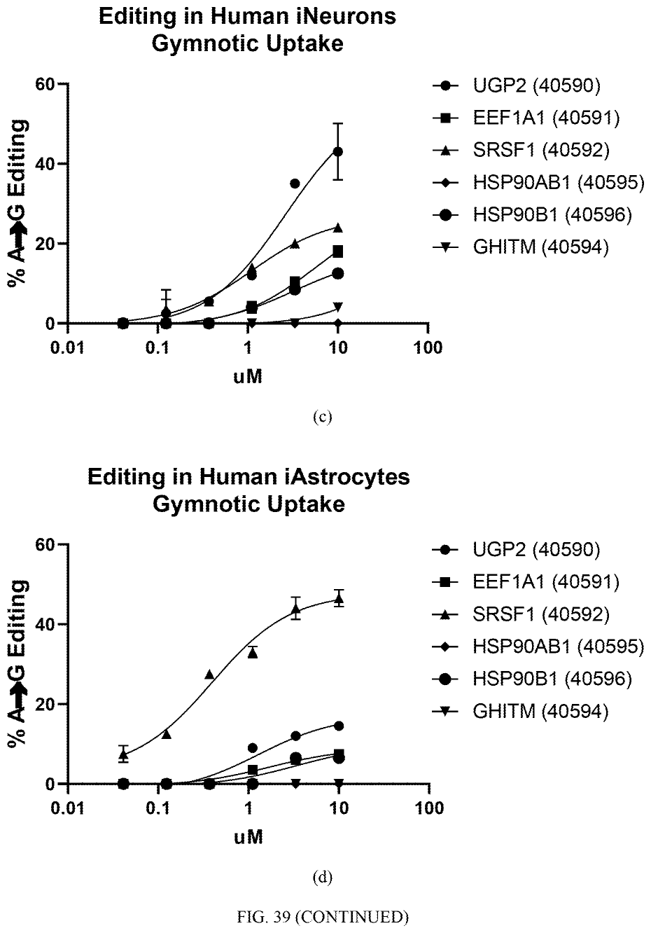

As described, the present disclosure provides technologies that can, among other things, provide editing in various systems, e.g., various types of cells, tissues, organs, organisms, etc. Certain data are presented in FIG. 39, which confirms editing in human neuronal cells. iCell neurons and iCell astrocytes (a and b, respectively; may also be referred to as iNeurons and iAstrocytes, respectively; both available from Brainxell) were treated with indicated oligonucleotide compositions at the indicated concentrations by gymnotic delivery. As demonstrated in FIG. 39, (a), all 4 oligonucleotide compositions (WV-27404, WV-37317, WV-37318, and WV-37324) provided editing in human neurons. It was confirmed that in some embodiments, non-negatively charged internucleotidic linkages, e.g., n001, can be utilized in accordance with the present disclosure to improve editing levels (e.g., WV-37317, WV-37318, and WV-37324 provided higher levels of editing compared to WV-27404 at various concentrations in iCell neurons, (a)). Certain data for ACTB editing in iCell astrocytes were presented in FIG. 39, (b). Certain data for additional target adenosine editing in iCell neurons and iCell astrocytes and were presented in (c) and (d), respectively. Cells were treated with indicated oligonucleotide compositions at indicated concentrations by gymnotic delivery. iCell neurons and astrocytes were dosed with oligonucleotide compositions comprising WV-40590 (targeting UGP2), WV-40591 (targeting EEF1A1), WV-40592 (targeting SRSF1), WV-40595 (targeting HSP90AB1), WV-40596 (targeting HSP90B1), and WV-40594 (targeting GHI™), respectively, that each target their respective mRNA. Those skilled in the art reading the present disclosure will understand that, among other things, higher editing levels (e.g., than those presented in the Figures) may be achieved using provided technologies in accordance with the present disclosure. Among other things, FIG. 39 demonstrated that provided technologies can be effectively delivered by gymnotic delivery to neuronal cells and can provide high levels of editing in such cells for a period of at least several days (e.g., as shown in FIG. 39, at least 5 or 6 days).

### Example 55. Useful Technologies for Assessing Oligonucleotide Technologies

Among other things, the present disclosure provides technologies for assessing agents, e.g., oligonucleotides, and compositions thereof, for editing, e.g., A to I (G) editing. In some embodiments, the present disclosure provides technologies that are useful for assessing agents (e.g., oligonucleotides) and compositions thereof that interact with, and/or modulate or utilize one or more functions of an ADAR polypeptide as described herein, e.g., an ADAR1 polypeptide. In some embodiments, the present disclosure provides non-human animal cells and/or non-human animals engineered to comprise and/or express an ADAR1 polypeptide or a characteristic portion thereof, or polynucleotide encoding an ADAR1 polypeptide or a characteristic portion thereof. In some embodiments, an ADAR1 polypeptide or a characteristic portion thereof is or comprises a primate ADAR1 or a characteristic portion thereof. In some embodiments, an ADAR1 polypeptide or a characteristic portion thereof is or comprises a primate ADAR1. In some embodiments, an ADAR1 polypeptide or a characteristic portion thereof is a primate ADAR1. In some embodiments, a primate is a non-human primate. In some embodiments, a primate is human. In some embodiments, an ADAR1 polypeptide or a characteristic portion thereof is or comprises human p110 ADAR1 or a characteristic portion thereof. In some embodiments, an ADAR1 polypeptide or a characteristic portion thereof is or comprises human p110 ADAR1. In some embodiments, an ADAR1 polypeptide or a characteristic portion thereof is human p110 ADAR1. In some embodiments, an ADAR1 polypeptide or a characteristic portion thereof is or comprises human p150 ADAR1 or a characteristic portion thereof. In some embodiments, an ADAR1 polypeptide or a characteristic portion thereof is or comprises human p150 ADAR1. In some embodiments, an ADAR1 polypeptide or a characteristic portion thereof is human p150 ADAR1. In some embodiments, a non-human animal is a rodent. In some embodiments, it is a rat. In some embodiments, it is a mouse. In some embodiments, the present disclosure provides mouse engineered to express human ADAR1. In some embodiments, the present disclosure provides mouse cells engineered to express human ADAR1.

Among other things, the present Example demonstrates that provided technologies are particularly useful for assessing agents, e.g., oligonucleotides, and compositions thereof that are useful for editing, e.g., adenosine editing described in the Examples. Among other things, the present disclosure provides and the present Example confirms that various agents (e.g., oligonucleotides) and compositions thereof that can provide editing in various human cells may show no or much lower levels of editing in certain cells (e.g., mouse cells) and certain animals such as rodents (e.g., mice) that do not contain or express human ADAR, e.g., human ADAR1; particularly, mice, a commonly used animal model, may be of limited uses for assessing various agents (e.g., oligonucleotides) for editing in humans, as agents active in human may show no or very low levels of activity (see FIG. 40 and FIG. 47, data for wild-type (WT) mice and cells, human cells, and cells and mice engineered to express hADAR1 p110 (huADAR mouse)). In some embodiments, the present disclosure provides cells and non-human animals (e.g., rodents such as mice) engineered to express human ADAR1 (e.g., human ADAR1 p110, p150, etc.), and their uses for assessing editing agents such as oligonucleotides and compositions thereof. Among other things, such engineered cells and/or animals can demonstrate activities that are more correlated with and/or predictive of activities in human cells than cells and/or animals not so engineered.

Generation of non-human mice expressing human ADAR1. Various technologies can be utilized in accordance with the present disclosure to provide mice engineered to express human ADAR1 polypeptide or a characteristic portion thereof. Certain useful technologies are described, e.g., Example 62 as examples.

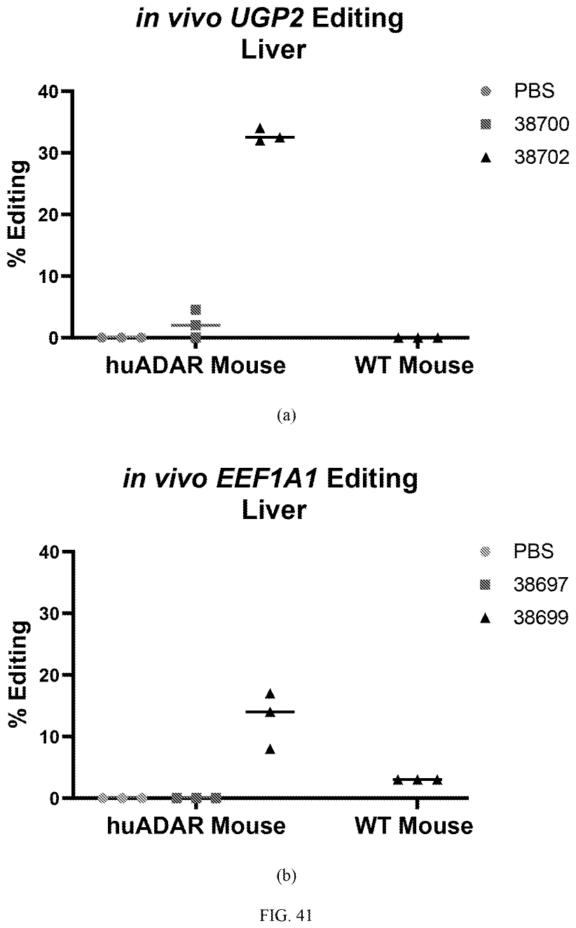

As demonstrated in FIG. 40, FIG. 41 and FIG. 47, in mouse cells (FIG. 40 and FIG. 47) and animals (FIG. 41) engineered to express human ADAR1, various oligonucleotides showed activity profiles that were much similar to their activity profiles in human cells compared to reference mouse cells and animals not engineered to express human ADAR1, for example, many oligonucleotides showed no or much lower levels of activity in reference mouse cells and animals not engineered to express human ADAR1 compared to human cells expressing human ADAR1 and/or mouse cells and animals engineered to express human ADAR1.

### Example 56. Provided Technologies Comprising Chiral Control and Various Internucleotidic Linkages can Provide High Editing Activities In Vitro and In Vivo

Among other things, the present Example confirms that chiral control and various types of internucleotidic linkages can be utilized to effectively improve editing efficiency. As shown in FIG. 40, FIG. 41 and FIG. 47, chirally controlled oligonucleotide compositions (e.g., WV-38700 and WV-38702 in FIG. 40, (a), and WV-38697 and WV-38699 in FIG. 40, (b)) can provide higher editing efficiency compared to reference non-chirally controlled oligonucleotide compositions (e.g., WV-38701 and WV-38698 in FIG. 40, (a) and (b), respectively; see also FIG. 47). Among other things, the present Example confirms that incorporation of non-negatively charged internucleotidic linkages, e.g., n001, in accordance with the present disclosure can also improve editing efficiency (WV-38702 in FIG. 40, (a) and FIG. 41, (a), and WV-38699 in FIG. 40, (b) and FIG. 41, (b); see also FIG. 47) in vivo and in vitro. Cells were dosed with compositions of GalNAc-conjugated oligonucleotides targeting UGP (WV-38700, WV-38700 and WV-38702) or EEF1A1 (WV-38697, WV-38697 and WV-38699). For FIG. 41, animals were dosed with indicated oligonucleotide compositions by subcutaneous (SC) administration at 10 mg/kg every other day for a total of 3 doses (days 1, 3, and 5) in C57/Blk6 mice that engineered to endogenously express human ADAR-p110 (human ADAR-p110 mice). The same dosing regimen was used to dose WV-38702 and WV-38699 compositions in wild-type C57/Blk6 mice. Livers from all mice were collected 3 days post last dose (day 8). Among other things, the present Example confirms that in some embodiments, chirally controlled oligonucleotide compositions, particularly of oligonucleotides comprising non-negatively charged internucleotidic linkages (e.g., n001), can provide improved editing efficiency in accordance with the present disclosure.

### Example 58. Provided Technologies can Provide Editing Activities In Vivo

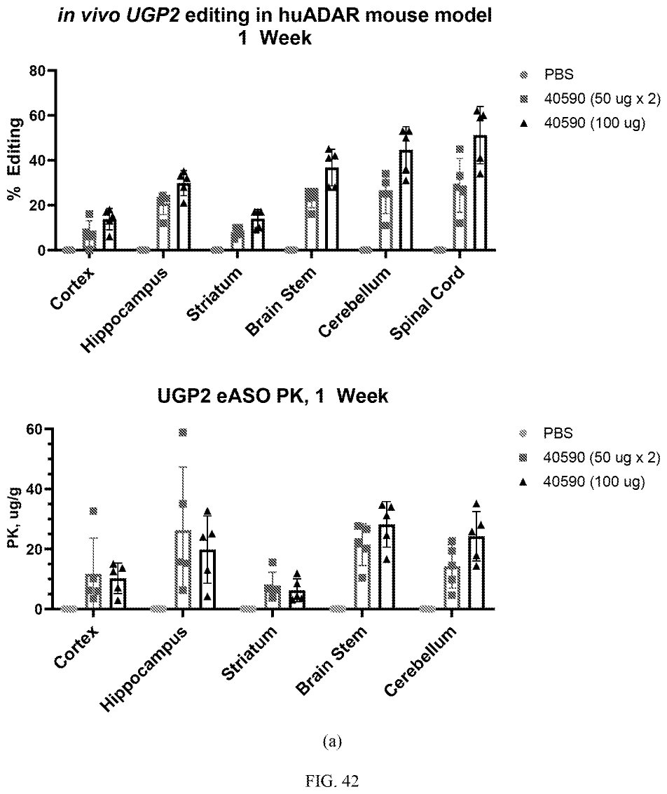

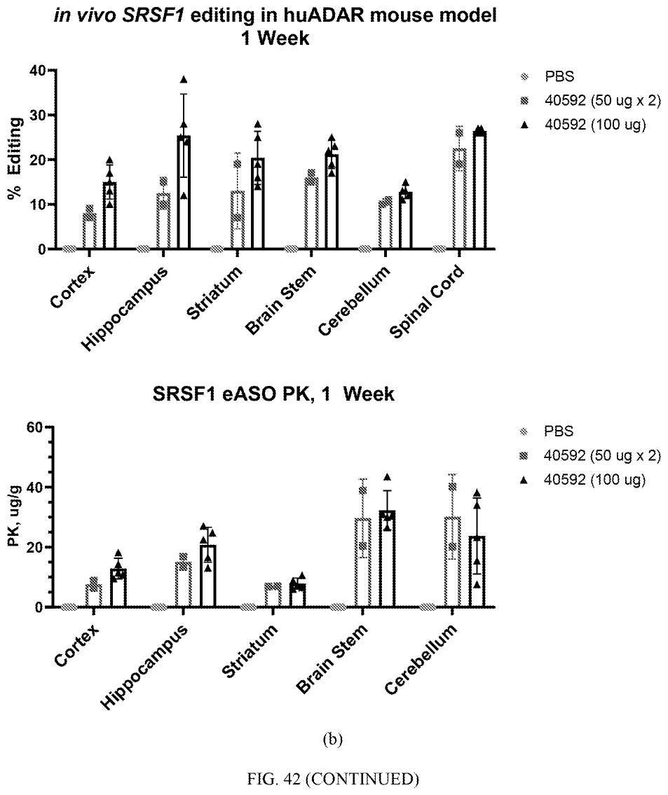

In some embodiments, the present disclosure provides oligonucleotide compositions that can, among other things, provide editing activities in various systems, e.g., in various cells, tissues, and/or organs in vivo. Certain data are presented in FIG. 42, confirming that provided technologies can provide editing in various tissues in vivo, including in CNS. C57/Blk6 mice engineered to endogenously express human Adar-p110 (human ADAR-p110 mice) were dosed by intracerebroventricular (ICV) administration of indicated oligonucleotide compositions at either a single 100 ug dose or 2×50 ug doses, spaced 2 days apart. 8 days after the single dose and 6 days after the last 50 ug dose, multiple CNS tissues were collected and analyzed. As can be seen in FIG. 42, UGP2 oligonucleotides (WV-40590) was present in all brain regions analyzed, with concentrations ranging from 5-60 ug/g of tissue (a). UGP2 mRNA was edited in all tissues analyzed (cortex, cerebellum, striatum, hippocampus, brain stem, and spinal cord) with percent editing ranging from 10-60%. For another target SRSF1, oligonucleotides (WV-40592) were observed in all brain regions analyzed with concentrations ranging from 5-45 ug/g of tissue (b), and SRSF1 mRNA showed editing in all tissues analyzed (cortex, cerebellum, striatum, hippocampus, brain stem, and spinal cord) with percent editing ranging from 10-40%. Among other things, the present Example confirms that provided technologies can provide editing in various tissues in vivo, including CNS, and can provide editing for at least a week after a last dose.

### Example 59. Activities of Certain Compositions May Correlate with Levels of Certain ADAR Polypeptides

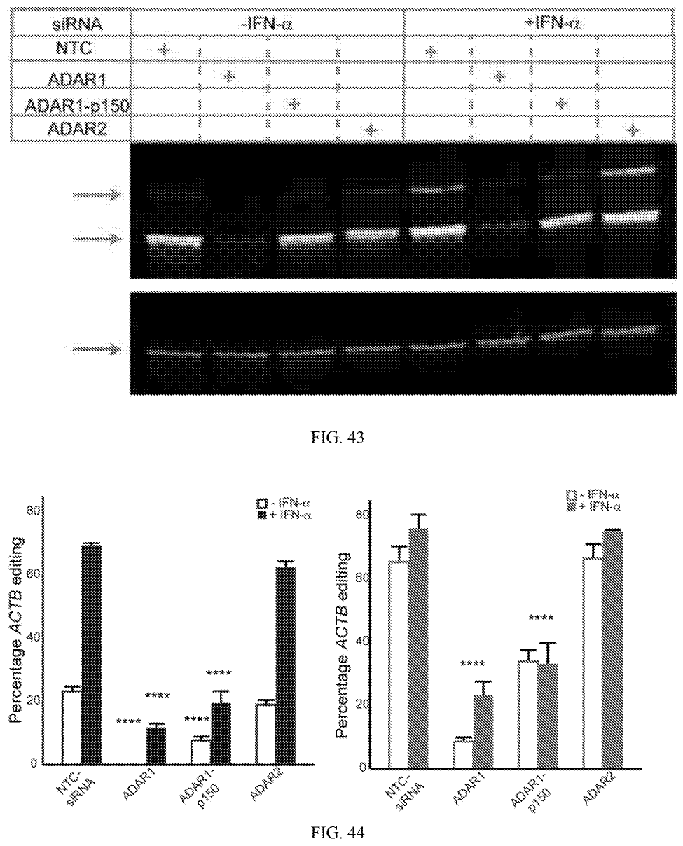

As described herein, provided technologies may utilize various polypeptides to provide editing. Without the intention to be limited by any theory, in some embodiments, provided technologies utilize ADAR1 to provide editing; in some embodiments, provided technologies utilize ADAR1 p110 to provide editing; in some embodiments, provided technologies utilize ADAR1 p150 to provide editing; in some embodiments, provided technologies utilize ADAR2 to provide editing; in some embodiments, levels of one or more ADAR polypeptides and/or isoforms thereof may be more associated with editing levels than one or more others; in some embodiments, provided technologies utilize two or more ADAR proteins (e.g., ADAR1, ADAR2, etc.) and/or isoforms thereof (e.g., p110 and/or p150 of ADAR1); in some embodiments, provided technologies utilize both p110 and p150, and optionally other isoforms, of ADAR1. In some embodiments, editing levels are associated with levels of ADAR1. In some embodiments, editing levels are associated with levels of ADAR1 p110. In some embodiments, editing levels are associated with levels of ADAR1 p150. In some embodiments, editing levels are associated more with levels of ADAR1 p110 than levels of ADAR1 p150 (e.g., as observed for certain chirally controlled oligonucleotide compositions). In some embodiments, editing levels are associated more with levels of ADAR1 p150 than levels of ADAR1 p110 (e.g., as observed for certain stereorandom oligonucleotide compositions). Among other things, the present Example provides data showing certain observed association between certain editing levels of certain oligonucleotide compositions and levels of certain proteins (and/or isoforms thereof) in certain circumstances. In some embodiments, small interfering RNA (siRNA) reagents targeting ADAR1 (both isoforms), ADAR1 p150, or ADAR2 were utilized to assess editing activities when levels of certain proteins were reduced (FIG. 43 and FIG. 44). Oligonucleotide compositions were transfected into cells. As shown, IFN-a treatment can increase levels of ADAR1 p150 expression in ARPE-19 cells. With a stereorandom oligonucleotide composition WV-23928, increased level of editing was observed following IFN-a stimulation compared to non-IFN-a treatment (FIG. 44, left panel, NTC-siRNA). Knockdown of ADAR1 (p110 and p150) or ADAR1 p150 alone reduced editing including with IFN-a stimulation. Without the intention to be limited by theory, editing by WV-23928 may be associated with levels of ADAR1 p150, and in some embodiments, may be more so than with levels of p110 and/or than that by WV-27395. Without the intention to be limited by theory, editing by chirally controlled oligonucleotide composition WV-27395 may be less associated with (or in some embodiments, not significantly associated with) levels of ADAR1 p150 or IFN-a treatment, and/or may be more associated with levels of ADAR1 p110, compared to WV-23928. In some embodiments, IFN-α-treatment did not significantly affect editing levels provided by WV-27395 (FIG. 44, right panel). ADAR2 protein was not detected at significant levels under these tested conditions, and siRNA-mediated depletion of ADAR2 did not seem to significantly impact on editing efficiency of WV-23928 or WV-27395 (FIG. 44).

### Example 60. Provided Technologies can Provide High Specificity

Among other things, provided technologies can provide high specificity. The present Example provides certain data as examples confirming such specificity.

In some embodiments, to assess specificity, deep RNA sequencing (RNA-seq) using strand-specific libraries was conducted to quantify on-target editing and off-target editing. In some embodiments, chirally controlled oligonucleotide composition WV-27458 and stereorandom composition WV-30298 were assessed in primary human hepatocytes. In some embodiments, it was observed that percentages of on-target edits detected with RNA-seq were well correlated (R²=0.996) to the percentages detected with Sanger sequencing. Also, higher percentage of ACTB editing was detected with WV-27458 (53.8%) than with WV-30298 (31.9%, FIG. 4b). No editing was detected anywhere else in the ACTB transcript.

To assess off-target editing for the whole transcriptome, Mutect2 was utilized to call edit sites. In some embodiments, Mutect 2 can provide, among other things, sensitivity in detecting low frequency variants and ability to prefilter variants that were in both mock and treated samples. Most variants detected that were specific to treated samples were in repetitive regions (e.g., Alu-repeat regions), and similar to other reports, these variants were filtered out. In some embodiments, provided technologies can direct sequence-specific editing to target transcripts without disrupting natural editing homeostasis (e.g., as evidenced by various variants identified in Alu-repeat regions). With filters applied, 178 variants were identified in the WV-30298-treated samples and 169 variants in the WV-27458-treated samples that were not found in mock-treated samples. These variants are potential sites where off-target editing could occur.

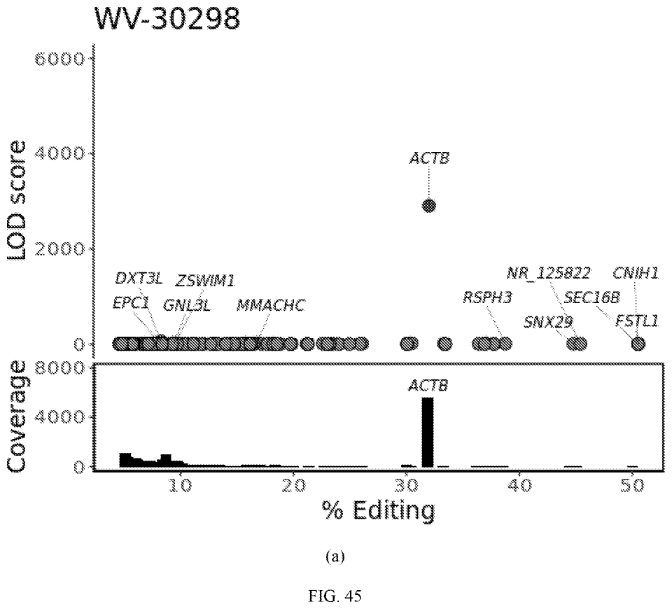

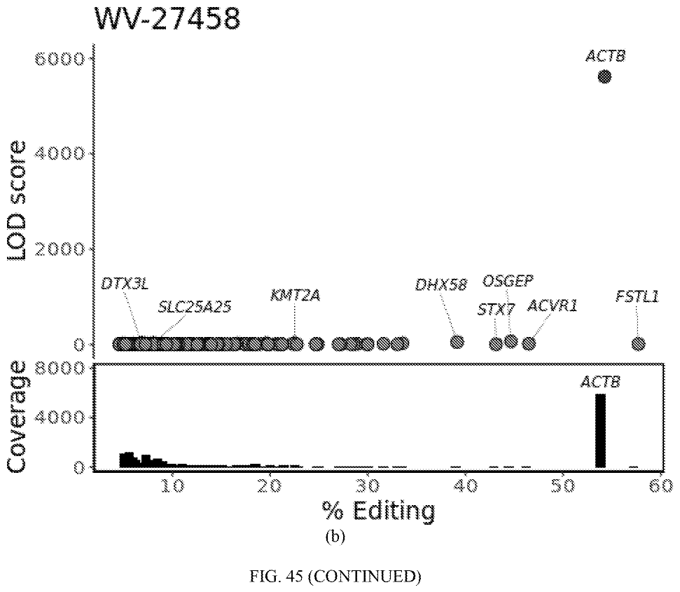

Most of these variants map to 3-untranslated regions; all variants had much lower LOD scores than the targeted ACTB site (40-fold lower for ACT-69, 80-fold for WV-27458). Additionally, most potential off-target edits had either low read coverage (WV-27458: mean coverage of editing >10%=45, mean coverage <10%=139; WV-30298: mean coverage >10%=43, mean coverage <10%=143) or occurred at low percentages (<10% of reads), indicating that both WV-30298 and WV-27458 elicited highly specific editing activity (FIG. 45). There was little overlap (24) between off-target sites detected with WV-30298 (145) and WV-27458 (154), and sequences around off-target sites were not related to the target sequence in ACTB, indicating that off-target editing, if any, may not be sequence dependent. Of the 24 shared off-target sites, most had high coverage with a low percentage of editing, indicating that they are relatively rare events. Among other things, data presented herein confirm that provided technologies can provide highly specific A to I editing of target adenosines.

### Example 61. Provided Technologies can Provide Multiplex Editing

Among other things, provided technologies can provide editing of several targets in a system at the same time. An example is described below.

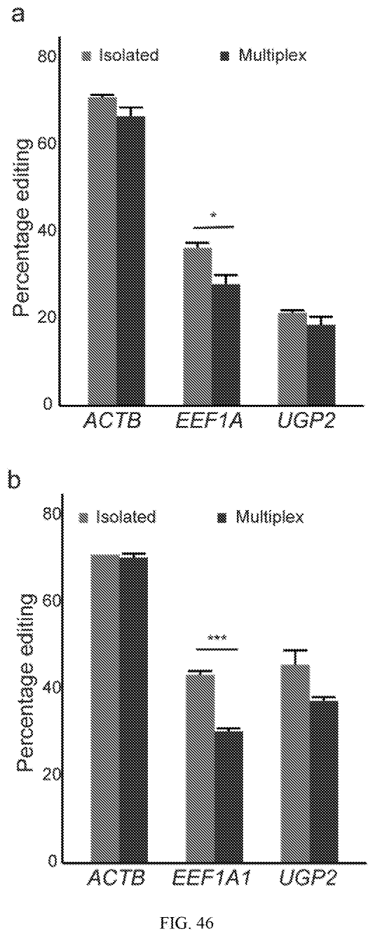

In one experiment, transcripts of three genes in primary human hepatocytes were edited under multiplex conditions. As demonstrated, efficient editing of transcripts of all three genes was achieved after transfection. In some embodiments, editing of EEF1A transcripts may be decreased under multiplex conditions compared with in isolation (FIG. 46, (a), Welch's t test, P<0.05), but still quite high editing level can be achieved. Multiplex editing was also achieved when using oligonucleotides comprising GalNAc (FIG. 46, (b)). Among other things, these data demonstrate that provided technologies can provide effective multiplex editing, in some instance as in the present example, without exogenous ADAR.

### Example 62. Provided Animal Models Confirm In Vivo Editing Activities

As described herein, various technologies may be utilized to assess provided technologies. Among other things, the present Example describes certain such useful technologies as examples. Those skilled in the art appreciate that in accordance with the present disclosure, various parameters, conditions, etc., may be adjusted to assess provided technologies, e.g., oligonucleotide compositions targeting various adenosines.

DNA expression constructs. In some embodiments, dual-luciferase reporter was used to assess RNA editing. In some embodiments, it was synthesized by Genscript (USA) and was based on prior reports (e.g., Cox, D. B. T. et al. RNA editing with CRISPR-Cas13. Science (New York, N.Y.) 358, 1019-1027, doi:10.1126/science.aaq0180 (2017)). In some embodiments, plasmids encoding human ADAR1 p110 (NM_001025107), human ADAR1 p150 (NM_001111), and human ADAR2 (NM_001112) were synthesized de novo (GenScript, USA).

Cell lines. Various cells may be useful for assessing provided technologies. In some embodiments, cells are commercially available. In some embodiments, cells may be prepared according to reported procedures. For example, ARPE-19 cells may be obtained from ATCC (CRL-2302, Manassas, Va.), primary human retinal pigmented epithelial cells (RPEs) from Lonza (Catalog No. 00194987, Basel, Switzerland), primary human hepatocytes from Thermo Fisher (Catalog No. HMCPUS, USA), and primary human bronchial epithelial cells from Lonza (Catalog No. CC-2540).

Luciferase reporter assays. In some embodiments, the present disclosure provides and utilizes luciferase reporter assays to assess provided editing agents, e.g., oligonucleotides, or compositions thereof. A useful procedure is described below as an example.

293T cells, seeded at 700,000 cells per well in a 6-well dish, were transfected 16 hr post-seeding with 1.25 ug of luciferase reporter construct and ADAR1- or ADAR2-expression vectors using 5 uL Lipofectamine 2000 (Thermo Fisher, USA) in OptiMEM according to manufacturer's protocol. The next morning, cells were replenished with fresh complete media. Eight hours later, cells were detached and reverse transfected in 96-well plates. Oligonucleotides and Lipofectamine 2000 (Thermo Fisher, USA) 0.25 uL/well) were each diluted in OptiMEM to a final volume of 10 uL in separate tubes. After 5 min, the two dilutions were mixed and incubated for 20 min before adding them to 130 uL of cell suspension (20,000 cells/well) in media lacking antibiotics. Luciferase activity was measured 48-96 hr later using Pierce Gaussia or Cypridina Luciferase Glow Assay Kits (Thermo Fisher, Cat. No. 16161 and 16171, respectively). Two 20 uL aliquots of media containing secreted luciferase was used to measure luciferase activity (20 uL per luciferase assay) according to manufacturer's protocol. cLuc activity was normalized to gLuc activity from the same well, and all data was standardized to a mock-transfection control (no oligonucleotides). In some embodiments, experiments were performed as biological replicates, n=2.

siRNA and IFN-alpha (IFN-α) studies. In some embodiments, such technologies are utilized to assess provided technologies. One experiment is described below as an example. ARPE-19 cells, seeded at 250,000 cells per well in a 6-well dish, were transfected with 20 nM of the appropriate siRNA and 7 uL of Lipofectamine RNAiMAX (Life Technologies). The siRNAs and RNAiMAX were each diluted into OptiMEM to a final volume of 250 uL in separate tubes. After 5 min, the two dilutions were mixed and incubated for 20 min before being evenly distributed to ARPE-19 cells in 1.5 mL of complete medium. 24 hr later, cells were detached and plated into 96-well plates (12,000 cells/well) for oligonucleotide transfection or 6-well plates to verify protein knockdown. 16 hr later, cells in the 96-well plates were transfected with oligonucleotide. Oligonucleotides and Lipofectamine RNAiMAX (0.3 uL/well) were diluted into OptiMEM to a final volume of 10 uL in separate tubes. After 5 min, the two dilutions were mixed and incubated for 20 min before adding them to cells in 100 uL of freshly replenished media. IFN-a (Millipore, Cat. No. IF007) was added to the media at a final concentration of 6000 U/mL at the time of oligonucleotide transfection. siRNAs were from Thermo Fisher and targeted ADAR1 (both isoforms; ADAR1Silencer Select siRNA, Cat. No. 4390824 s1008), ADAR1 p150 (Custom Silencer Select siRNA, sense strand: 5′-GCCUCGCGGGCGCAAUGAAtt (SEQ ID NO: 870); antisense strand: 5′-UUCAUUGCGCCCGCGAGGCat (SEQ ID NO: 871)), ADAR2 (ADARBI Silencer Select siRNA, Cat. No. 4392420 s1012), or non-targeting control (Silencer Select Negative Control No. 1, Cat. No. 4390843).

Westernblot. In some embodiments, western blot is utilized. Those skilled in the art appreciate that various protocols may be utilized in accordance with the present disclosure. In one example, for western blot cells were washed with PBS once, and lysed in RIPA buffer (Thermo, Cat. No. 89900) containing protease inhibitor. Cleared lysates were transferred to fresh tubes and total protein was quantified using BCA protein assay kit according to manufacturer's protocol. Samples were denatured by heating at 90° C. for 5 min in XT sample buffer with reducing agent. 7 ug of protein per sample were loaded on Criterion XT 4-12% Protein Gel and resolved with XT MOPS running buffer. Proteins were transferred to a nitrocellulose membrane using BioRad Trans-Blot Turbo system. The membrane was blocked for 1 h in 5% milk solution and stained overnight at 4° C. with primary antibody diluted 1:1,000 in TBST (anti-ADAR1: Cell Signaling Catalog No. 1417; anti-vinculin: Invitrogen Cat. No. MA5-11690). Membranes were washed and incubated for 1 h at room temperature with secondary antibodies (Donkey anti-Rabbit-IRDye 800CW and Goat anti-mouse-IRDye680LT, respectively) diluted 1:10,000 in PBS, 5% non-fat milk. After washing, the membranes were visualized on the ODYSSEY CLx Imaging System. The intensity of protein bands was analyzed with Image Studio Lite version 5.2 software.

Endogenous RNA-editing assays. In some embodiments, provided technologies are assessed utilizing endogenous RNA-editing assays. A useful protocol is described here as an example. To assess editing of endogenous transcripts under transfection conditions, primary hepatocytes (10,000-20,000 cells per well) were seeded in 96-well plates. 16 hrs later, oligonucleotides (50 nM final concentration) were transfected using Lipofectamine RNAiMax (0.3 uL/well). Oligonucleotides and Lipofectamine were diluted to a final volume of 10 uL in separate tubes. After 5 min, the two dilutions were mixed and incubated for another 20 min before adding them to cells in 100 uL of media. RNA was harvested 48 hrs later. For GalNAc-mediated uptake, oligonucleotides were added to the media at the desired concentration at the time hepatocytes were seeded (10,000-hepatocytes per well; 96-well plate). For gymnotic uptake, NHBE cells were seeded in 96-well plates (5,000 cells/well), and 16 hrs later cells were treated with fresh media containing oligonucleotides at the desired concentration. RNA was collected 48 hrs later, e.g., utilizing protocols described below.

In some embodiments, RNA was harvested from cells using Promega SV 96 Total RNA Isolation System according to manufacturer's protocol. RNA was eluted in a final volume of 40-50 uL. 9 uL of RNA was used to generated cDNA using a High-Capacity cDNA Reverse Transcription Kit (Life Technologies, Cat. No. 4374967) in a 20 uL reaction according to manufacturer's protocol. 2 uL of cDNA was used to amplify the transcript of interest using Phusion High-Fidelity DNA Polymerase (Thermo Fisher, Cat. No. F530) and transcript appropriate primers. Certain useful primers were listed below as examples; those skilled in the art appreciate that other primers may be utilized for the same and other genes. PCR products were purified using AMPure XP beads (Beckman Coulter), and analyzed by Sanger sequencing (Genewiz, USA). A-to-I edits were quantified using EditR software (https://moriaritylab. shinyapps.io/editr_v10/).

### Human

| | SEQ | | SEQ | |
|---|---|---|---|---|
| Transcript | ID NO | Forward primer | ID NO | Reverse primer |
| ACTB | 872 | 5′- | 873 | 5′- |
| | | CGAGCATCCCCCAAAGTTCAC- | | CACTCCCAGGGAGACCAAAAG |
| | | 3′ | | C-3′ |
| EEF1A1 | 874 | 5′- | 875 | 5′- |
| | | GGTGTTGGTGAATTTGAAGCT | | GTCAGTTGGACGAGTTGGTGG- |
| | | GG-3′ | | 3′ |
| EIF4H | 876 | 5′- | 877 | 5′- |
| | | CCCCTACACAGCATACGTAGG- | | CCTTTTCTGAATCCAAAGCCAC |
| | | 3′ | | C-3′ |
| HSP90B1 | 878 | 5′- | 879 | 5′- |
| | | CGCTGATCAGAGACATGCTTC | | CTTCTTCATCTGTTCCCACATC |
| | | G-3′ | | C-3′ |
| SRSF1 | 880 | 5′- | 881 | 5′- |
| | | TCCTAACATCTACATTCCCTTC | | CTAAGCACTGTGACAAATTAG |
| | | G-3′ | | CC-3′ |
| UGP2 | 882 | 5′- | 883 | 5′- |
| | | ACAGAAGCTTGGTACCCTCC-3′ | | TGTGCTTTTGGCACTTGAGC-3′ |

### Mouse

| | SEQ | | SEQ | |
|---|---|---|---|---|
| Transcript | ID NO | Forward primer | ID NO | Reverse primer |
| UGP2 | 884 | CCTCCAGGGCATGGAGATATC | 885 | CCATGTCAATGGCATTCTGTTC |
| | | | | C |
| EEF1A1 | 886 | CACCGAGCCACCATACAGTC | 887 | GAACACCAGTCTCCACTCGG |
| SRSF1 | 888 | CTACATTCCCCTCATGTCTTTG | 889 | GATCTGATCCTCCTCAATCCTC |
| | | G | | C |

Transcriptomes sequencing to assess editing specificity. In some embodiments, transcriptome sequencing was utilized to assess specificity. A useful protocol is described below as an example:

Oligonucleotide treatment and RNA harvesting. For off-target editing analysis, primary human hepatocytes were seeded in 6-well plates and treated with oligonucleotide compositions as described above (1 uM final concentration). 48 hrs later, cells were washed with PBS and collected in 1 mL of Trizol/well. 200 uL of chloroform was added to each sample, mixed thoroughly and allowed to incubate for 10 mins, before spinning down at 13,000 g for 10 mins. Aqueous phase was transferred to Promega SV 96 Total RNA Isolation System columns and RNA was extracted according to manufacturer's protocol. 2 wells used per biological replicate to ensure sufficient RNA quality and amount.

Total RNA was ribosome depleted and strand-specific libraries were generated (Genewiz, USA). 50 M reads (2×150 bp) per sample were sequenced with the Illumina NovaSeq platform (Illumina, San Diego, Calif., USA). Adaptor sequences were removed with using BBTools BB Decontamination using Kmers (BBDuk) trimming (https://jgi.doe.gov/data-and-tools/bbtools/bb-tools-user-guide/). Before variant calling, reads were processed using GATK best practices for calling variants in RNA-sequencing data (https://gatk.broadinstitute.org/hc/en-us/articles/360035531192-RNAseq-short-variant-discovery-SNPs-Indels-). Briefly, trimmed reads were aligned to genome assembly GRCh38 using STAR aligner 2-pass (https://www.ncbi.nlm.nih.gov/assembly/GCF_000001405.26/). Picard's AddOrReplaceReadGroups was used to change group names of mock sample and treatment samples, and Picard's MarkDuplicates was used to remove PCR duplicate reads. GATK's SplitNCigar and BaseRecalbiatrion was used to format RNA reads over introns and used to detect any systemic bias that could arise during library prep. For variant calling, Mutect2 was used to detect variants unique to treated samples. For each comparison, all three treated replicates were equivalent to tumor samples and the three mock replicates were equivalent to normal samples. After FilterMutectCalls, only variants that passed were kept. TLOD was used to determine the significance of the detected variants. To filter known ADAR editing sites, Alu-repeat regions were filtered from the Mutect2 output. Alu-repeat regions were taken from UCSC Table Browser (https://genome.ucsc.edu/cgi-bin/hgTables). Variants with <5% editing were removed. Annovar was used to annotate the remaining variants.

Statistics. Various statistic methods may be utilized in accordance with the present disclosure. In some embodiments, the following statistic methods were used. All statistical analyses and plots were generated in the R computing environment (v 3.6.0) and the KNIME (Konstanz Information Miner) platform (v 4.1.3). For all statistical comparisons, unequal variance was assumed. Two-tailed Welch's t-tests and Welch's one-way ANOVAs were utilized for comparing two means and multiple means across one factor, respectively. For two-factor analyses, a white-adjusted two-way ANOVA was performed to correct for unequal variance using the car R package (v 3.0-7). Following ANOVA analysis, two-tailed pairwise and Dunnett post-hoc comparisons were utilized when applicable using the multcomp R package (v 1.4-13), with P values adjusted for multiple hypotheses through single-step method via a multivariate t distribution (R package mvtnorm, v 1.1-0) and using robust HC3 covariance estimation to adjust for heteroscedasticity (R package sandwich, v 2.5-1). For dose response curves, three-parameter log-logistic functions were fit and absolute EC50 values were calculated using the R package dre (v 3.0-1). Remaining plots were generated using R package ggplot2 (v. 3.3.0).

Generation of mice engineered to express human ADAR1. Various technologies can be utilized to generate engineered mice expressing human ADAR1. For example, certain useful technologies are described in US U.S. Ser. No. 10/314,297B2. One example was described below.

A human ADAR-1 isoform p110 (e.g., transcript variant 4) polynucleotide was synthesized and confirmed with Sanger sequencing. A human ADAR-1 isoform p150 (transcript variant 1) polynucleotide was also synthesized and confirmed with Sanger sequencing. The fragments were digested with restriction enzymes and ligated into targeting vectors. Those skilled in the art appreciate various vectors may be utilized. In some embodiments, a vector comprises selection marker, e.g., an ampicillin resistance positive selection marker, an origin site for replication, and in a 5′ to 3′ order: a 5′ (aka left) homology arm, an adenovirus derived splice acceptor, a KOZAK sequence, the coding sequence of interest, a Woodchuck Hepatitis Virus Posttranscriptional Regulatory Element (WPRE), a bovine growth hormone (bGH) polyadenylation signal, and a 3′ (aka right) homology arm. Certain sequences are presented below as examples; other sequences for the same purpose may also be utilized:

5′ homology arm (SEQ ID NO: 890):
GGAAGAGTCCTGACCCAGGGAAGACATTAAAAAGGTAGTGGGGTCGACTAGATGAAGGAGAGCCTTTCTC
TCTGGGCAAGAGCGGTGCAATGGTGTGTAAAGGTAGCTGAGAAGACGAAAAGGGCAAGCATCTTCCTGCT
ACCAGGCTGGGGAGGCCCAGGCCCACGACCCCGAGGAGAGGGAACGCAGGGAGACTGAGGTGACCCTTCT
TTCCCCCGGGGCCCGGTCGTGTGGTTCGGTGTCTCTTTTCTGTTGGACCCTTACCTTGACCCAGGCGCTG
CCGGGGCCTGGGCCCGGGCTGCGGCGCACGGCACTCCCGGGAGGCAGCGAGACTCGAGTTAGGCCCAACG
CGGCGCCACGGCGTTTCCTGGCCGGGAATGGCCCGTACCCGTGAGGTGGGGGTGGGGGGCAGAAAAGGCG
GAGCGAGCCCGAGGCGGGGAGGGGGAGGGCCAGGGGCGGAGGGGGCCGGCACTACTGTGTTGGCGGACTG
GCGGGACTAGGGCTGCGTGAGTCTCTGAGCGCAGGCGGGCGGCGGCCGCCCCTCCCCCGGCGGCGGCAGC
GGCGGCAGCGGCGGCAGCTCACTCAGCCCGCTGCCCGAGCGGAAACGCCACTGACCGCACGGGGATTCCC
AGTGCCGGCGCCAGGGGCACGCGGGACACGCCCCCTCCCGCCGCGCCATTGGCCTCTCCGCCCACCGCCC
CACACTTATTGGCCGGTGCGCCGCCAATCAGCGGAGGCTGCCGGGGCCGCCTAAAGAAGAGGCTGTGCTT
TGGGGCTCCGGCTCCTCAGAGAGCCTCGGCTAGGTAGGGGATCGGGACTCTGGCGGGAGGGCGGCTTGGT
GCGTTTGCGGGGATGGGCGGCCGCGGCAGGCCCTCCGAGCGTGGTGGAGCCGTTCTGTGAGACAGCCGGG
TACGAGTCGTGACGCTGGAAGGGGCAAGCGGGTGGTGGGCAGGAATGCGGTCCGCCCTGCAGCAACCGGA
GGGGGAGGGAGAAGGGAGCGGAAAAGTCTCCACCGGACGCGGCCATGGCTCGGGGGGGGGGGGGCAGCGG
AGGAGCGCTTCCGGCCGACGTCTCGTCGCTGATTGGCTTCTTTTCCTCCCGCCGTGTGTGAAAACACAAA
TGGCGTGTTTTGGTTGGCGTAAGGCGCCTGTCAGTTAACGGCAGCCGGAGTGCGCAGCCGCCGGCAGCCT
CGCTCTGCCCACTGGGTGGGGCGGGAGGTAGGTGGGGTGAGGCGAGCTGGACGTGCGGGCGCGGTCGGCC
TCTGGCGGGGCGGGGGAGGGGAGGGAGGGTCAGCGAAAGTAGCTCGCGCGCGAGCGGCCGCCCACCCTCC
CCTTCCTCTGGGGGAGTCGTTTTACCCGCCGCCGGCCGGGCCTCGTCGTCTGATTGGCTCTCGGGGCCCA
GAAAACTGGCCCTTGCCATTGGCTCGTGTTCGTGCAAGTTGAGTCCATCCGCCGGCCAGCGGGGGCGGCG
AGGAGGCGCTCCCAGGTTCCGGCCCTCCCCTCGGCCCCGCGCCGCAGAGTCTGGCCGCGCGCCCCTGCGC
AACGTGGCAGGAAGCGCGCGCTGGGGGCGGGGACGGGCAGTAGGGCTGAGCGGCTGCGGGGCGGGTGCAA
GCACGTTTCCGACTTGAGTTGCCTCAAGAGGGGCGTGCTGAGCCAGACCTCCATCGCGCACTCCGGGGAG
TGGAGGGAAGGAGCGAGGGCTCAGTTGGGCTGTTTTGGAGGCAGGAAGCACTTGCTCTCCCAAAGTCGCT
CTGAGTTGTTATCAGTAAGGGAGCTGCAGTGGAGTAGGCGGGGAGAAGGCCGCACCCTTCTC
Splice acceptor (SEQ ID NO: 891):
gtgacctgcacgtctagggcgcagtagtccagggtttccttgatgatgtcatacttatcctgtccctttt
ttttccacagctcgcggttgaggacaaactcttcgcggtctttccagt
human ADAR1 p110 (SEQ ID NO: 892):
ATGGCCGAGATCAAGGAGAAAATCTGCGACTATCTCTTCAATGTGTCTGACTCCTCTGCCCTGAATTTGG
CTAAAAATATTGGCCTTACCAAGGCCCGAGATATAAATGCTGTGCTAATTGACATGGAAAGGCAGGGGGA
TGTCTATAGACAAGGGACAACCCCTCCCATATGGCATTTGACAGACAAGAAGCGAGAGAGGATGCAAATC
AAGAGAAATACGAACAGTGTTCCTGAAACCGCTCCAGCTGCAATCCCTGAGACCAAAAGAAACGCAGAGT
TCCTCACCTGTAATATACCCACATCAAATGCCTCAAATAACATGGTAACCACAGAAAAAGTGGAGAATGG
GCAGGAACCTGTCATAAAGTTAGAAAACAGGCAAGAGGCCAGACCAGAACCAGCAAGACTGAAACCACCT
GTTCATTACAATGGCCCCTCAAAAGCAGGGTATGTTGACTTTGAAAATGGCCAGTGGGCCACAGATGACA
TCCCAGATGACTTGAATAGTATCCGCGCAGCACCAGGTGAGTTTCGAGCCATCATGGAGATGCCCTCCTT
CTACAGTCATGGCTTGCCACGGTGTTCACCCTACAAGAAACTGACAGAGTGCCAGCTGAAGAACCCCATC
AGCGGGCTGTTAGAATATGCCCAGTTCGCTAGTCAAACCTGTGAGTTCAACATGATAGAGCAGAGTGGAC
CACCCCATGAACCTCGATTTAAATTCCAGGTTGTCATCAATGGCCGAGAGTTTCCCCCAGCTGAAGCTGG
AAGCAAGAAAGTGGCCAAGCAGGATGCAGCTATGAAAGCCATGACAATTCTGCTAGAGGAAGCCAAAGCC
AAGGACAGTGGAAAATCAGAAGAATCATCCCACTATTCCACAGAGAAAGAATCAGAGAAGACTGCAGAGT
CCCAGACCCCCACCCCTTCAGCCACATCCTTCTTTTCTGGGAAGAGCCCCGTCACCACACTGCTTGAGTG
TATGCACAAATTGGGGAACTCCTGCGAATTCCGTCTCCTGTCCAAAGAAGGCCCTGCCCATGAACCCAAG
TTCCAATACTGTGTTGCAGTGGGAGCCCAAACTTTCCCCAGTGTGAGTGCTCCCAGCAAGAAAGTGGCAA
AGCAGATGGCCGCAGAGGAAGCCATGAAGGCCCTGCATGGGGAGGCGACCAACTCCATGGCTTCTGATAA
CCAGCCTGAAGGTATGATCTCAGAGTCACTTGATAACTTGGAATCCATGATGCCCAACAAGGTCAGGAAG
ATTGGCGAGCTCGTGAGATACCTGAACACCAACCCTGTGGGTGGCCTTTTGGAGTACGCCCGCTCCCATG
GCTTTGCTGCTGAATTCAAGTTGGTCGACCAGTCCGGACCTCCTCACGAGCCCAAGTTCGTTTACCAAGC
AAAAGTTGGGGGTCGCTGGTTCCCAGCCGTCTGCGCACACAGCAAGAAGCAAGGCAAGCAGGAAGCAGCA
GATGCGGCTCTCCGTGTCTTGATTGGGGAGAACGAGAAGGCAGAACGCATGGGTTTCACAGAGGTAACCC
CAGTGACAGGGGCCAGTCTCAGAAGAACTATGCTCCTCCTCTCAAGGTCCCCAGAAGCACAGCCAAAGAC
ACTCCCTCTCACTGGCAGCACCTTCCATGACCAGATAGCCATGCTGAGCCACCGGTGCTTCAACACTCTG
ACTAACAGCTTCCAGCCCTCCTTGCTCGGCCGCAAGATTCTGGCCGCCATCATTATGAAAAAAGACTCTG
AGGACATGGGTGTCGTCGTCAGCTTGGGAACAGGGAATCGCTGTGTAAAAGGAGATTCTCTCAGCCTAAA
AGGAGAAACTGTCAATGACTGCCATGCAGAAATAATCTCCCGGAGAGGCTTCATCAGGTTTCTCTACAGT
GAGTTAATGAAATACAACTCCCAGACTGCGAAGGATAGTATATTTGAACCTGCTAAGGGAGGAGAAAAGC
TCCAAATAAAAAAGACTGTGTCATTCCATCTGTATATCAGCACTGCTCCGTGTGGAGATGGCGCCCTCTT
TGACAAGTCCTGCAGCGACCGTGCTATGGAAAGCACAGAATCCCGCCACTACCCTGTCTTCGAGAATCCC
AAACAAGGAAAGCTCCGCACCAAGGTGGAGAACGGAGAAGGCACAATCCCTGTGGAATCCAGTGACATTG
TGCCTACGTGGGATGGCATTCGGCTCGGGGAGAGACTCCGTACCATGTCCTGTAGTGACAAAATCCTACG
CTGGAACGTGCTGGGCCTGCAAGGGGCACTGTTGACCCACTTCCTGCAGCCCATTTATCTCAAATCTGTC
ACATTGGGTTACCTTTTCAGCCAAGGGCATCTGACCCGTGCTATTTGCTGTCGTGTGACAAGAGATGGGA
GTGCATTTGAGGATGGACTACGACATCCCTTTATTGTCAACCACCCCAAGGTTGGCAGAGTCAGCATATA
TGATTCCAAAAGGCAATCCGGGAAGACTAAGGAGACAAGCGTCAACTGGTGTCTGGCTGATGGCTATGAC
CTGGAGATCCTGGACGGTACCAGAGGCACTGTGGATGGGCCACGGAATGAATTGTCCCGGGTCTCCAAAA
AGAACATTTTTCTTCTATTTAAGAAGCTCTGCTCCTTCCGTTACCGCAGGGATCTACTGAGACTCTCCTA
TGGTGAGGCCAAGAAAGCTGCCCGTGACTACGAGACGGCCAAGAACTACTTCAAAAAAGGCCTGAAGGAT
ATGGGCTATGGGAACTGGATTAGCAAACCCCAGGAGGAAAAGAACTTTTATCTCTGCCCAGTATAG
human ADAR1 p150 (SEQ ID NO: 893):
ATGAATCCGCGGCAGGGGTATTCCCTCAGCGGATACTACACCCATCCATTTCAAGGCTATGAGCACAGAC
AGCTCAGATACCAGCAGCCTGGGCCAGGATCTTCCCCCAGTAGTTTCCTGCTTAAGCAAATAGAATTTCT
CAAGGGGCAGCTCCCAGAAGCACCGGTGATTGGAAAGCAGACACCGTCACTGCCACCTTCCCTCCCAGGA
CTCCGGCCAAGGTTTCCAGTACTACTTGCCTCCAGTACCAGAGGCAGGCAAGTGGACATCAGGGGTGTCC
CCAGGGGCGTGCATCTCGGAAGTCAGGGGCTCCAGAGAGGGTTCCAGCATCCTTCACCACGTGGCAGGAG
TCTGCCACAGAGAGGTGTTGATTGCCTTTCCTCACATTTCCAGGAACTGAGTATCTACCAAGATCAGGAA
CAAAGGATCTTAAAGTTCCTGGAAGAGCTTGGGGAAGGGAAGGCCACCACAGCACATGATCTGTCTGGGA
AACTTGGGACTCCGAAGAAAGAAATCAATCGAGTTTTATACTCCCTGGCAAAGAAGGGCAAGCTACAGAA
AGAGGCAGGAACACCCCCTTTGTGGAAAATCGCGGTCTCCACTCAGGCTTGGAACCAGCACAGCGGAGTG
GTAAGACCAGACGGTCATAGCCAAGGAGCCCCAAACTCAGACCCGAGTTTGGAACCGGAAGACAGAAACT
CCACATCTGTCTCAGAAGATCTTCTTGAGCCTTTTATTGCAGTCTCAGCTCAGGCTTGGAACCAGCACAG
CGGAGTGGTAAGACCAGACAGTCATAGCCAAGGATCCCCAAACTCAGACCCAGGTTTGGAACCTGAAGAC
AGCAACTCCACATCTGCCTTGGAAGATCCTCTTGAGTTTTTAGACATGGCCGAGATCAAGGAGAAAATCT
GCGACTATCTCTTCAATGTGTCTGACTCCTCTGCCCTGAATTTGGCTAAAAATATTGGCCTTACCAAGGC
CCGAGATATAAATGCTGTGCTAATTGACATGGAAAGGCAGGGGGATGTCTATAGACAAGGGACAACCCCT
CCCATATGGCATTTGACAGACAAGAAGCGAGAGAGGATGCAAATCAAGAGAAATACGAACAGTGTTCCTG
AAACCGCTCCAGCTGCAATCCCTGAGACCAAAAGAAACGCAGAGTTCCTCACCTGTAATATACCCACATC
AAATGCCTCAAATAACATGGTAACCACAGAAAAAGTGGAGAATGGGCAGGAACCTGTCATAAAGTTAGAA
AACAGGCAAGAGGCCAGACCAGAACCAGCAAGACTGAAACCACCTGTTCATTACAATGGCCCCTCAAAAG
CAGGGTATGTTGACTTTGAAAATGGCCAGTGGGCCACAGATGACATCCCAGATGACTTGAATAGTATCCG
CGCAGCACCAGGTGAGTTTCGAGCCATCATGGAGATGCCCTCCTTCTACAGTCATGGCTTGCCACGGTGT
TCACCCTACAAGAAACTGACAGAGTGCCAGCTGAAGAACCCCATCAGCGGGCTGTTAGAATATGCCCAGT
TCGCTAGTCAAACCTGTGAGTTCAACATGATAGAGCAGAGTGGACCACCCCATGAACCTCGATTTAAATT
CCAGGTTGTCATCAATGGCCGAGAGTTTCCCCCAGCTGAAGCTGGAAGCAAGAAAGTGGCCAAGCAGGAT
GCAGCTATGAAAGCCATGACAATTCTGCTAGAGGAAGCCAAAGCCAAGGACAGTGGAAAATCAGAAGAAT
CATCCCACTATTCCACAGAGAAAGAATCAGAGAAGACTGCAGAGTCCCAGACCCCCACCCCTTCAGCCAC
ATCCTTCTTTTCTGGGAAGAGCCCCGTCACCACACTGCTTGAGTGTATGCACAAATTGGGGAACTCCTGC
GAATTCCGTCTCCTGTCCAAAGAAGGCCCTGCCCATGAACCCAAGTTCCAATACTGTGTTGCAGTGGGAG
CCCAAACTTTCCCCAGTGTGAGTGCTCCCAGCAAGAAAGTGGCAAAGCAGATGGCCGCAGAGGAAGCCAT
GAAGGCCCTGCATGGGGAGGCGACCAACTCCATGGCTTCTGATAACCAGCCTGAAGGTATGATCTCAGAG
TCACTTGATAACTTGGAATCCATGATGCCCAACAAGGTCAGGAAGATTGGCGAGCTCGTGAGATACCTGA
ACACCAACCCTGTGGGTGGCCTTTTGGAGTACGCCCGCTCCCATGGCTTTGCTGCTGAATTCAAGTTGGT
CGACCAGTCCGGACCTCCTCACGAGCCCAAGTTCGTTTACCAAGCAAAAGTTGGGGGTCGCTGGTTCCCA
GCCGTCTGCGCACACAGCAAGAAGCAAGGCAAGCAGGAAGCAGCAGATGCGGCTCTCCGTGTCTTGATTG
GGGAGAACGAGAAGGCAGAACGCATGGGTTTCACAGAGGTAACCCCAGTGACAGGGGCCAGTCTCAGAAG
AACTATGCTCCTCCTCTCAAGGTCCCCAGAAGCACAGCCAAAGACACTCCCTCTCACTGGCAGCACCTTC
CATGACCAGATAGCCATGCTGAGCCACCGGTGCTTCAACACTCTGACTAACAGCTTCCAGCCCTCCTTGC
TCGGCCGCAAGATTCTGGCCGCCATCATTATGAAAAAAGACTCTGAGGACATGGGTGTCGTCGTCAGCTT
GGGAACAGGGAATCGCTGTGTGAAAGGAGATTCTCTCAGCCTAAAAGGAGAAACTGTCAATGACTGCCAT
GCAGAAATAATCTCCCGGAGAGGCTTCATCAGGTTTCTCTACAGTGAGTTAATGAAATACAACTCCCAGA
CTGCGAAGGATAGTATATTTGAACCTGCTAAGGGAGGAGAAAAGCTCCAAATAAAAAAGACTGTGTCATT
CCATCTGTATATCAGCACTGCTCCGTGTGGAGATGGCGCCCTCTTTGACAAGTCCTGCAGCGACCGTGCT
ATGGAAAGCACAGAATCCCGCCACTACCCTGTCTTCGAGAATCCCAAACAAGGAAAGCTCCGCACCAAGG
TGGAGAACGGAGAAGGCACAATCCCTGTGGAATCCAGTGACATTGTGCCTACGTGGGATGGCATTCGGCT
CGGGGAGAGACTCCGTACCATGTCCTGTAGTGACAAAATCCTACGCTGGAACGTGCTGGGCCTGCAAGGG
GCACTGTTGACCCACTTCCTGCAGCCCATTTATCTCAAATCTGTCACATTGGGTTACCTTTTCAGCCAAG
GGCATCTGACCCGTGCTATTTGCTGTCGTGTGACAAGAGATGGGAGTGCATTTGAGGATGGACTACGACA
TCCCTTTATTGTCAACCACCCCAAGGTTGGCAGAGTCAGCATATATGATTCCAAAAGGCAATCCGGGAAG
ACTAAGGAGACAAGCGTCAACTGGTGTCTGGCTGATGGCTATGACCTGGAGATCCTGGACGGTACCAGAG
GCACTGTGGATGGGCCACGGAATGAATTGTCCCGGGTCTCCAAAAAGAACATTTTTCTTCTATTTAAGAA
GCTCTGCTCCTTCCGTTACCGCAGGGATCTACTGAGACTCTCCTATGGTGAGGCCAAGAAAGCTGCCCGT
GACTACGAGACGGCCAAGAACTACTTCAAAAAAGGCCTGAAGGATATGGGCTATGGGAACTGGATTAGCA
AACCCCAGGAGGAAAAGAACTTTTATCTCTGCCCAGTATAG
Woodchuck Hepatitis Virus Posttranscriptional Regulatory Element (WPRE)
(SEQ ID NO: 894):
AATCAACCTCTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCATTTTCTCCTC
CTTGTATAAATCCTGGTTGCTGTCTCTTTATGAGGAGTTGTGGCCCGTTGTCAGGCAACGTGGCGTGGTG
TGCACTGTGTTTGCTGACGCAACCCCCACTGGTTGGGGCATTGCCACCACCTGTCAGCTCCTTTCCGGGA
CTTTCGCTTTCCCCCTCCCTATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCATCGTCCTTTCCTTGGCTGCTC
GCCTGTGTTGCCACCTGGATTCTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCTTCGCCTTCGCCCTCAGACGAG
TCGGATCTCCCTTTGGGCCGCCTCCCCGC
polyadenylation signal (SEQ ID NO: 895):
CTGTGCCTTCTAGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATGGCAGGCATGCTGGGGA
3′ (aka right) homology arm (SEQ ID NO: 896):
CGGAGGGGGGAGGGGAGTGTTGCAATACCTTTCTGGGAGTTCTCTGCTGCCTCCTGGCTTCTGAGGACCG
CCCTGGGCCTGGGAGAATCCCTTCCCCCTCTTCCCTCGTGATCTGCAACTCCAGTCTTTCTAGAAGATGG
GCGGGAGTCTTCTGGGCAGGCTTAAAGGCTAACCTGGTGTGTGGGCGTTGTCCTGCAGGGGAATTGAACA
GGTGTAAAATTGGAGGGACAAGACTTCCCACAGATTTTCGGTTTTGTCGGGAAGTTTTTTAATAGGGGCA
AATAAGGAAAATGGGAGGATAGGTAGTCATCTGGGGTTTTATGCAGCAAAACTACAGGTTATTATTGCTT
GTGATCCGCCTCGGAGTATTTTCCATCGAGGTAGATTAAAGACATGCTCACCCGAGTTTTATACTCTCCT
GCTTGAGATCCTTACTACAGTATGAAATTACAGTGTCGCGAGTTAGACTATGTAAGCAGAATTTTAATCA
TTTTTAAAGAGCCCAGTACTTCATATCCATTTCTCCCGCTCCTTCTGCAGCCTTATCAAAAGGTATTTTA
GAACACTCATTTTAGCCCCATTTTCATTTATTATACTGGCTTATCCAACCCCTAGACAGAGCATTGGCAT
TTTCCCTTTCCTGATCTTAGAAGTCTGATGACTCATGAAACCAGACAGATTAGTTACATACACCACAAAT
CGAGGCTGTAGCTGGGGCCTCAACACTGCAGTTCTTTTATAACTCCTTAGTACACTTTTTGTTGATCCTT
TGCCTTGATCCTTAATTTTCAGTGTCTATCACCTCTCCCGTCAGGTGGTGTTCCACATTTGGGCCTATTC
TCAGTCCAGGGAGTTTTACAACAATAGATGTATTGAGAATCCAACCTAAAGCTTAACTTTCCACTCCCAT
GAATGCCTCTCTCCTTTTTCTCCATTTATAAACTGAGCTATTAACCATTAATGGTTTCCAGGTGGATGTC
TCCTCCCCCAATATTACCTGATGTATCTTACATATTGCCAGGCTGATATTTTAAGACATTAAAAGGTATA
TTTCATTATTGAGCCACATGGTATTGATTACTGCTTACTAAAATTTTGTCATTGTACACATCTGTAAAAG
GTGGTTCCTTTTGGAATGCAAAGTTCAGGTGTTTGTTGTCTTTCCTGACCTAAGGTCTTGTGAGCTTGTA
TTTTTTGTATTTAAGGAGTGGTTTGTGTTGGAGTGGGTTGAGTGATGGGATTGTAGAGGTTATTGGTGGT
CTAAATGTGATTTTGCCAAGCTTCTTCAGGACCTATAATTTTGCTTGACTTGTAGCCAAACACAAGTAAA
ATGATTAAGCAACAAATGTATTTGTGAAGCTTGGTTTTTAGGTTGTTGTGTTGTGTGTGCTTGTGCTCTA
TAATAATACTATCCAGGGGCTGGAGAGGTGGCTCGGAGTTCAAGAGCACAGACTGCTCTTCCAGAAGTCC
TGAGTTCAATTCCCAGCAACCACATGGTGGCTCACAACCATCTGTAATGGGATCTGATGCCCTCTTCTGG
TGTGTCTGAAGACCACAAGTGTATTCACATTAAATAAATAAATCCTCCTTCTTCTTCTTTTTTTTTTTTT
TAAAGAGAATACTGTCTCCAGTAGAATTTACTGAAGTAATGAAATACTTTGTGTTTGTTCCAATATGGTA
GCCAATAATCAAATTACTCTTTAAGCACTGGAAATGTTACCAAGGAACTAATTTTTATTTGAAGTGTAAC
TGTGGACAGAGGAGCCATAACTGCAGACT

In some embodiments, a vector is propogated and linearized to provide polynucleotide for injection.

Various technologies may be utilized in accordance with the present disclosure to introduce polynucleotide encoding an ADAR1 polypeptide or a characteristic portion thereof into genomes. In some embodiments, commercially available Extreme Genome Editing (EGE™) System was utilized. After injection of polynucleotides into zygotes, zygotes were then transferred into surrogate mothers. Pups were genotyped, in some embodiments, by PCR and Sanger sequencing, to identify and confirm insertion of polynucleotides of interest.

Identified founder pups were utilized to establish stable germine transmission of polynucleotide of interest using available methods in accordance with the present disclosure. For example, in some embodiments, founder pups heterozygous for huADAR1 p110 or p150 insertions were crossed with C57BL/6J mice to produce stable F1 progeny. In one cross, a WT male C57BL/6J animal was mated with a confirmed allele carrier female, and the resultant pups were genotyped using PCR. In another cross, a number of WT female C57BL/6J were mated with a confirmed allele carrier male, and the resultant pups were genotyped using PCR to confirm insertion. These F1 animals were additionally assessed by southern blot genotyping to confirm presence of polynucleotide of interest, e.g., a polynucleotide encoding huADAR1 p110. Homozygous engineered animals can be created through standard breeding techniques in accordance with the present disclosure. For example, heterozygous animals comprising a polynucleotide whose sequence encodes human ADAR1 p110 were mated to produce multiple litters of pups, initially available genotyping data indicated multiple WT, multiple heterozygotes, and multiple homozygotes. Expression of introduced polynucleotide was confirmed by western blot in various tissues. For example, expression of human ADAR1 p110 was confirmed to in lung, hepatocytes, cerebellum, pons/medulla, cortex, and midbrain and in various cases at similar levels to primary human cells, e.g., human hepatocytes (for hepatocytes) and human iCell neurons (for the neuronal tissues).

### Example 63. Certain Technologies for Preparing Provided Compounds Including Various Oligonucleotides

As described herein, the present disclosure provides various technologies for preparing provided compounds including various oligonucleotides and compositions thereof. In some embodiments, one or more or all internucleotidic linkages in provided oligonucleotides are chirally controlled. In some embodiments, provided compounds are chirally controlled. In some embodiments, provided compounds are chirally pure. Described below are preparation of certain useful compounds as examples.

### Synthesis of (2R,3S,5R)-2-((bis(4-methoxyphenyl)(phenyl)methoxy)methyl)-5-(2,6-dioxo-3,6-dihydropyrimidin-1(2H)-yl)tetrahydrofuran-3-yl (2-cyanoethyl) diisopropylphosphoramidite

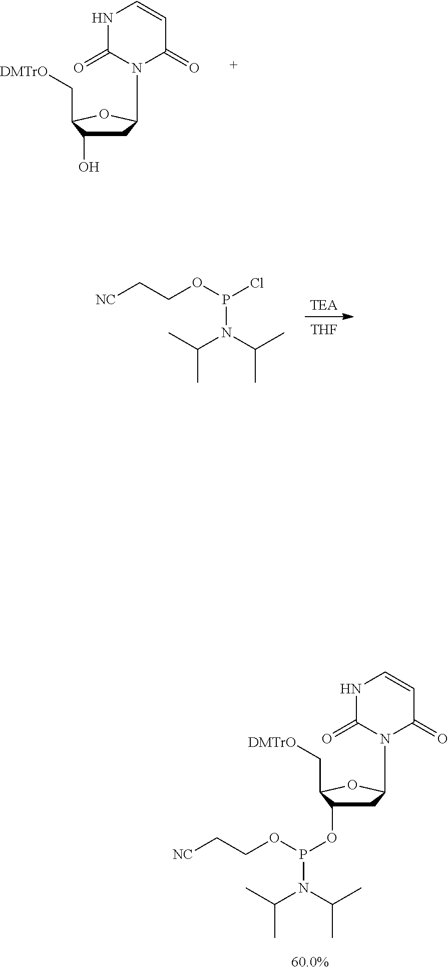

To a solution of dry 3-[(2R,4R,5R)-5-[[bis(4-methoxyphenyl)-phenyl-methoxy]methyl]-4-hydroxy-tetrahydrofuran-2-yl]-1H-pyrimidine-2,4-dione (476.5 mg, 0.90 mmol) in THF (6 mL) was added triethylamine (0.50 mL, 3.59 mmol). Cooled to 0° C. To the clear solution was added 2-cyanoethyl N,N-diisopropylchlorophosphoramidite (0.24 mL, 1.08 mmol) dropwise. The resulting white slurry was continued to stir at 0° C. for 1 hr then at rt for 1 hr 15 min. TLC and LCMS showed that only small amount of product was formed and starting material was major product. Additional triethylamine (0.45 mL) was added followed by additional 2-cyanoethyl N,N-diisopropylchlorophosphoramidite (0.22 mL) dropwise. The reaction mixture was continued to stir at rt for 2 hr. TLC showed the starting material was consumed. Triethylamine (0.6 mL) was added followed by anhydrous MgSO₄ (380 mg) and EtOAc (10 mL). The mixture was filtered, and the clear filtrate was concentrated to afford the crude product as a greasy stuff. The crude product was purified by normal phase column chromatography applying 10-100% EtOAc in hexanes (each mobile phase contained 5% triethylamine) as the gradient to afford the title compound as a white foam (0.394 g, 60.0% yield). ¹H NMR (600 MHz, CDCl₃) δ 9.13 (s, 1H), 7.50-7.43 (m, 2H), 7.35 (td, J=7.3, 1.5 Hz, 4H), 7.27-7.19 (m, 2H), 7.20-7.12 (m, 1H), 6.82-6.71 (m, 5H), 6.66 (dd, J=7.7, 5.4 Hz, 1H), 5.55 (dd, J=7.7, 0.8 Hz, 1H), 4.73-4.59 (m 1H), 4.15-4.06 (m, 1H), 3.83-3.67 (m, 7H), 3.60-3.52 (m, 1H), 3.50 (ddt, J=16.9, 13.6, 6.7 Hz, 1H), 3.36-3.30 (m, 1H), 2.85 (dddd, J=18.1, 13.2, 8.0, 5.1 Hz, 1H), 2.59 (dt, J=12.8, 6.3 Hz, 1H), 2.44-2.32 (m, 1H), 2.26 (dddd, J=33.7, 13.6, 8.6, 5.4 Hz, 1H), 1.27 (dd, J=8.5, 6.8 Hz, 2H), 1.15 (d, J=6.8 Hz, 3H), 1.12 (d, J=6.8 Hz, 6H), 1.01 (d, J=6.8 Hz, 3H); ³¹P NMR (243 MHz, CDCl₃) δ 148.45, 148.33; MS (ESI), 729.82 [M−H]⁻.

### Synthesis of 3-((2R,4S,5R)-5-((bis(4-methoxyphenyl)(phenyl)methoxy)methyl)-4-(((1S,3S,3aS)-3-((methyldiphenylsilyl)methyl)tetrahydro-1H,3H-pyrrolo[1,2-c][1,3,2]oxazaphosphol-1-yl)oxy)tetrahydrofuran-2-yl)pyrimidine-2,4(1H,3H)-dione

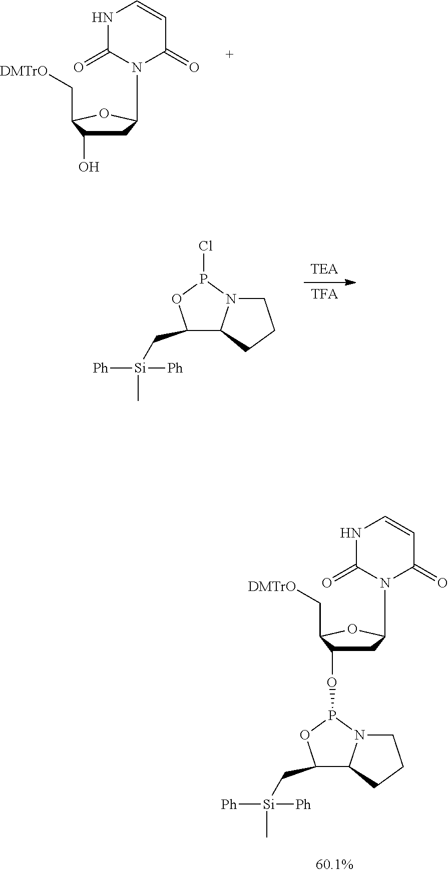

To a solution of dry 3-[(2R,4R,5R)-5-[[bis(4-methoxyphenyl)-phenyl-methoxy]methyl]-4-hydroxy-tetrahydrofuran-2-yl]-1H-pyrimidine-2,4-dione (695 mg, 1.31 mmol) in THF (9 mL) was added triethylamine (0.88 mL, 6.29 mmol). The solution was cooled to 0° C. [(3S,3aS)-1-chloro-3a,4,5,6-tetrahydro-3H-pyrrolo[1,2-c][1,3,2]oxazaphosphol-3-yl]methyl-methyl-diphenyl-silane (0.9574 Min THF, 2.19 mL, 2.1 mmol) was added dropwise. The resulting cloudy reaction solution was stirred at 0° C. for 2 hr then at rt for 2 hr. TLC and LCMS showed the reaction was not complete. Cooled to 0° C. again. Additional triethylamine (0.274 mL) was added followed by additional [(3S,3aS)-1-chloro-3a,4,5,6-tetrahydro-3H-pyrrolo[1,2-c][1,3,2]oxazaphosphol-3-yl]methyl-methyl-diphenyl-silane (0.9574M in THF, 0.684 mL) dropwise. The white slurry was continued to stir at 0° C. for 2.5 hr. TLC and LCMS showed only minor starting material still remained. The reaction was quenched by water (23 uL). Anhydrous MgSO4 (313 mg) was added. The mixture was filtered through celite, and the clear filtrate was concentrated to afford the crude product as an off-white foam. The crude product was purified by normal phase column chromatography applying 20-100% EtOAc in hexanes (each mobile phase contained 5% triethylamine) as the gradient to afford the title compound as a white foam (0.685 g, 60.1% yield). ¹H NMR (600 MHz, DMSO) δ 11.04 (s, 1H), 7.54-7.49 (m, 3H), 7.49-7.45 (m, 2H), 7.40-7.33 (m, 3H), 7.33-7.28 (m, 3H), 7.28-7.19 (m, 8H), 7.19-7.16 (m, 1H), 6.85-6.77 (m, 4H), 6.44 (dd, J=8.7, 4.2 Hz, 1H), 5.59 (d, J=7.6 Hz, 1H), 4.75-4.66 (m, 1H), 4.63 (dt, J=10.2, 5.3 Hz, 1H), 3.77-3.66 (m, 7H), 3.31-3.25 (m, 1H), 3.25-3.19 (m, 1H), 3.13 (dd, J=9.9, 7.7 Hz, 1H), 3.01 (ddd, J=17.9, 8.6, 4.5 Hz, 1H), 2.58-2.51 (m, 1H), 1.88-1.78 (m, 2H), 1.77-1.71 (m, 1H), 1.58-1.52 (m, 1H), 1.48-1.41 (m, 2H), 1.41-1.36 (m, 1H), 1.12 (dq, J=13.5, 11.4, 10.6 Hz, 1H), 0.58 (s, 3H); ³¹P NMR (243 MHz, DMSO) δ 142.16; MS (ESI), 868.37 [M−H]⁻.

### Synthesis of 1-((2R,4S,5R)-5-((bis(4-methoxyphenyl)(phenyl)methoxy)methyl)-4-hydroxytetrahydrofuran-2-yl)-1,3-dihydro-2H-imidazol-2-one

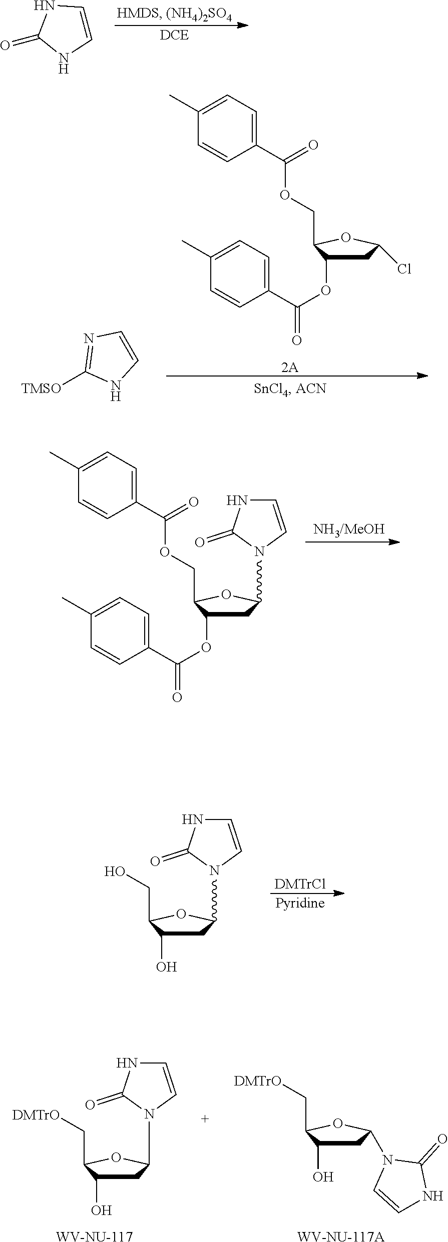

Step 1. Two batches, A mixture of 1,3-dihydro-2H-imidazol-2-one (20 g, 237.88 mmol, 1 eq.), (NH₄)₂SO₄ (2.40 g, 18.16 mmol, 1.36 mL, 7.64e-2 eq.) in HMDS (450 mL) and DCE (150 mL) was refluxed at 90° C. for 2 h. The reaction became clearly. The two batches were combined for workup. The excess HMDS and dichloroethane were removed from the mixture by evaporation. 2-((trimethylsilyl)oxy)-1H-imidazole (74.34 g, crude) was obtained as a white solid.

Step 2. Two batches: 2-((trimethylsilyl)oxy)-1H-imidazole (37 g, 236.79 mmol, 3 eq.) and (2R,3S,5R)-5-chloro-2-(((4-methylbenzoyl)oxy)methyl)tetrahydrofuran-3-yl 4-methylbenzoate (30.69 g, 78.93 mmol, 1 eq.) was dissolved in MeCN (390 mL). To the mixture was added a solution of SnCl₄ (4.11 g, 15.79 mmol, 1.84 mL, 0.2 eq.) in MeCN (10 mL) dropwise at −15° C., and the solution was stirred for 1 hr at 25° C. TLC (Petroleum ether: Ethyl acetate=1: 1, Rf=0.2) indicated (2R,3S,5R)-5-chloro-2-(((4-methylbenzoyl)oxy)methyl)tetrahydrofuran-3-yl 4-methylbenzoate was consumed completely and one main new spot formed. The reaction was clean according to TLC. The two batches were combined for work up: The reaction mixture was poured into a dichloromethane (600 mL) and saturated NaHCO₃ solution (400 mL). The organic layer was washed with a saturated sodium chloride solution (60 mL), dried with MgSO₄, and evaporated in vacuo. The crude was purified by column chromatography (SiO₂, Petroleum ether/Ethyl acetate=3/1 to 0/1). After purification the product could be seen at the TLC plate. Got the mixture of the ((2R,3S)-3-((4-methylbenzoyl)oxy)-5-(2-oxo-2,3-dihydro-1H-imidazol-1-yl)tetrahydrofuran-2-yl)methyl 4-methylbenzoate (25 g, 57.28 mmol, 36.29% yield) as a yellow oil. ¹H NMR (400 MHz, CHLOROFORM-d) 6=9.68-9.40 (m, 1H), 8.02-7.68 (m, 4H), 7.33-6.98 (m, 5H), 6.56-5.41 (m, 4H), 4.73-4.35 (m, 3H), 2.99-2.39 (m, 2H), 2.38-2.25 (m, 6H).

Step 3. Two batches: A solution of the mixture of ((2R,3S)-3-((4-methylbenzoyl)oxy)-5-(2-oxo-2,3-dihydro-1H-imidazol-1-yl)tetrahydrofuran-2-yl)methyl 4-methylbenzoate (12.5 g, 28.64 mmol, 1 eq.) in ammonia (600 mL), the mixture was stirred at 25° C. for 12 hr. TLC (Petroleum ether: Ethyl acetate=1/1, product Rf=0.0) indicated complete consumption and one new spot formed. The two batches were combined for work up: The reaction mixture was concentrated under reduced pressure to give yellow oil. Without further purification. 1-((4S,5R)-4-Hydroxy-5-(hydroxymethyl)tetrahydrofuran-2-yl)-1,3-dihydro-2H-imidazol-2-one (11.47 g, crude) was obtained as a yellow oil. LCMS: (M+H⁺: 201.1).

Step 4. Two batches: To a solution of 1-((4S,5R)-4-Hydroxy-5-(hydroxymethyl)tetrahydrofuran-2-yl)-1,3-dihydro-2H-imidazol-2-one mixture (5.73 g, 28.62 mmol, 1 eq.) in pyridine (150 mL) was added DMTCl (11.65 g, 34.35 mmol, 1.2 eq.). The mixture was stirred at 25° C. for 12 hr. LCMS showed the starting material was consumed completely and one main peak with desired mass was detected. The two batches were combined for work up: The resulting turbid solution was diluted with H₂O (400 mL) and extracted with EtOAc (800 mL). The combined extracts were washed with H₂O (100 mL) and brine (150 mL). The solution was dried (Na₂SO₄), filtered, and concentrated at reduced pressure. The crude compound was purified by MPLC (Petroleum ether/Ethyl acetate=3/1 to 0/1). 1-((2R,4S,5R)-5-((Bis(4-methoxyphenyl)(phenyl)methoxy)methyl)-4-hydroxytetrahydrofuran-2-yl)-1,3-dihydro-2H-imidazol-2-one (WV-NU-117) (9.5 g, 17.39 mmol, 30.38% yield, 92.001% purity) and WV-NU-117A (6.9 g, 12.62 mmol, 22.04% yield, 91.885% purity) were obtained as yellow solid. WV-NU-117: ¹H NMR (400 MHz, DMSO-d6) δ=10.01 (br s, 1H), 7.43-7.33 (m, 2H), 7.32-7.09 (m, 8H), 6.87 (br dd, J=1.8, 8.9 Hz, 4H), 6.41-6.29 (m, 2H), 5.84 (br t, J=6.9 Hz, 1H), 5.22 (d, J=4.5 Hz, 1H), 4.17 (br d, J=3.4 Hz, 1H), 3.85-3.76 (m, 1H), 3.73 (s, 6H), 3.06 (br d, J=4.9 Hz, 2H), 2.25-2.11 (m, 1H), 2.06-1.99 (m, 1H); ¹³C NMR (101 MHz, DMSO-d6) δ=158.53, 130.19, 128.27, 128.21, 113.64, 85.92, 55.50; LCMS (M−H⁺): 501.1, purity 92.0%. WV-NU-117A: ¹H NMR (400 MHz, DMSO-d6) δ=10.00 (br s, 1H), 7.45-7.22 (m, 9H), 6.90 (br d, J=8.6 Hz, 4H), 5.86 (br dd, J=4.2, 7.8 Hz, 1H), 5.48 (d, J=4.6 Hz, 1H), 4.13 (br dd, J=3.1, 7.1 Hz, 1H), 3.74 (s, 6H), 3.09-2.89 (m, 2H), 2.05-1.95 (m, 2H); ¹³C NMR (101 MHz, DMSO-d6) δ=158.53, 153.67, 136.11, 130.17, 128.32, 128.16, 113.69, 109.84, 108.72, 85.92, 85.65, 82.20, 71.60, 55.50; LCMS (M−H⁺): 501.2, purity 91.9%.

### Synthesis of (2R,3S,5R)-2-((bis(4-methoxyphenyl)(phenyl)methoxy)methyl)-5-(2-oxo-2,3-dihydro-1H-imidazol-1-yl)tetrahydrofuran-3-yl (2-cyanoethyl) diisopropylphosphoramidite

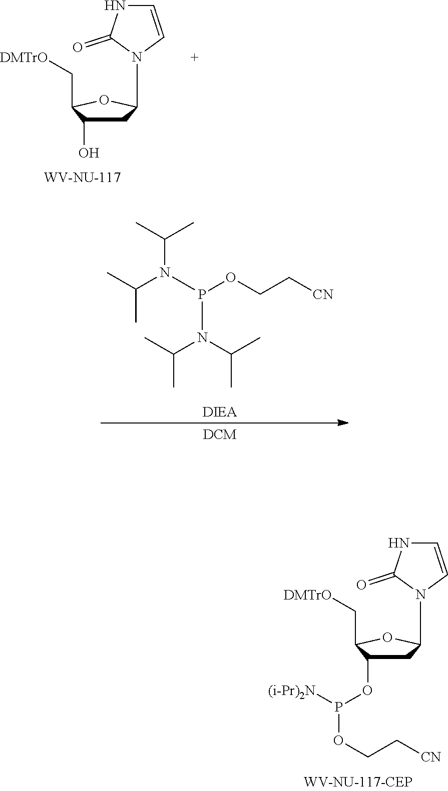

To a solution of WV-NU-117 (1.8 g, 3.58 mmol, 1 eq.) in DMF (20 mL) was added 5-ethylsulfanyl-2H-tetrazole (466.23 mg, 3.58 mmol, 1 eq.) and 1-methylimidazole (588.14 mg, 7.16 mmol, 571.01 uL, 2 eq.) then added 3-bis(diisopropylamino)phosphanyloxypropanenitrile (1.19 g, 3.94 mmol, 1.25 mL, 1.1 eq.) at 0° C. The mixture was stirred at 0-25° C. for 3 hr. TLC (Petroleum ether: Ethyl acetate=0: 1, Rf₁=0.55, Rf₂=0.50) indicated WV-NU-117 was consumed and three new spots formed. The reaction mixture was quenched by addition sat. NaHCO₃ aq. (50 mL) at 0° C., and extracted with EtOAc (20 mL*4). Dried over Na₂SO₄, filtered and concentrated under reduced pressure to give a residue. The residue was purified by column chromatography (SiO₂, Petroleum ether/Ethyl acetate=1/0 to 0/1, then ethyl acetate/acetonitrile=10:1, 5% TEA). (2R,3S,5R)-2-((bis(4-methoxyphenyl)(phenyl)methoxy)methyl)-5-(2-oxo-2,3-dihydro-1H-imidazol-1-yl)tetrahydrofuran-3-yl (2-cyanoethyl) diisopropylphosphoramidite (WV-NU-117-CEP) (0.8 g, 1.14 mmol, 31.78% yield) was obtained as a yellow oil. ¹H NMR (400 MHz, CHLOROFORM-d) 6=10.03-9.82 (m, 1H), 7.76 (s, 1H), 7.50-7.39 (m, 2H), 7.36-7.28 (m, 5H), 7.26-7.13 (m, 2H), 6.93-6.71 (m, 4H), 6.41-6.31 (m, 1H), 6.26-6.20 (m, 1H), 6.14-6.06 (m, 1H), 4.69-4.50 (m, 1H), 4.27-4.14 (m, 2H), 3.83-3.72 (m, 6H), 3.69-3.41 (m, 5H), 3.39-3.27 (m, 1H), 3.25-3.17 (m, 1H), 2.76 (br d, J=1.5 Hz, 2H), 2.66-2.56 (m, 1H), 2.47-2.29 (m, 3H), 1.28 (dd, J=5.8, 6.5 Hz, 12H), 1.21-1.12 (m, 8H), 1.07 (d, J=6.8 Hz, 2H); ³¹P NMR (162 MHZ, CHLOROFORM-d) δ 148.47, 148.26; LCMS: (M+H⁺)=703.3.

### Synthesis of 1-((2R,4S,5R)-5-((bis(4-methoxyphenyl)(phenyl)methoxy)methyl)-4-(((1S,3S,3aS)-3-((methyldiphenylsilyl)methyl)tetrahydro-1H,3H-pyrrolo[1,2-c][1,3,2]oxazaphosphol-1-yl)oxy)tetrahydrofuran-2-yl)-1,3-dihydro-2H-imidazol-2-one

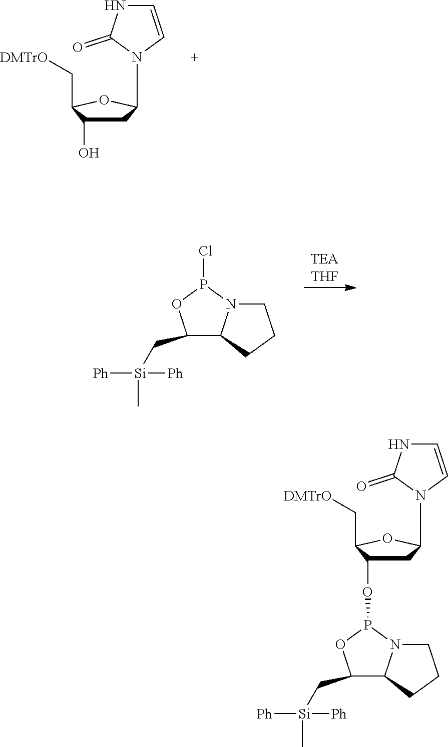

Dry 3-[(2R,4R,5R)-5-[[bis(4-methoxyphenyl)-phenyl-methoxy]methyl]-4-hydroxy-tetrahydrofuran-2-yl]-1H-imidazol-2-one (2.0 g, 3.98 mmol) in a rbf was dissolved in THF (26 mL). To the clear solution was added triethylamine (3.74 mL, 26.86 mmol). [(3S,3aS)-1-chloro-3a,4,5,6-tetrahydro-3H-pyrrolo[1,2-c][1,3,2]oxazaphosphol-3-yl]methyl-methyl-diphenyl-silane (0.96M solution in THF, 7.48 mL, 7.16 mmol) was added dropwise. The resulting cloudy reaction solution was stirred at rt for 2.5 hr. TLC and LCMS showed the reaction was complete. Anhydrous MgSO₄ (480 mg) was added. Stirred for 1 min. The mixture was filtered, and the filtrate was concentrated. The resulting crude product was purified by normal phase column chromatography applying 20-100% EtOAc in hexanes (each mobile phase contained 5% triethylamine) as the gradient to afford 1-((2R,4S,5R)-5-((bis(4-methoxyphenyl)(phenyl)methoxy)methyl)-4-(((1S,3S,3aS)-3-((methyldiphenylsilyl)methyl)tetrahydro-1H,3H-pyrrolo[1,2-c][1,3,2]oxazaphosphol-1-yl)oxy)tetrahydrofuran-2-yl)-1,3-dihydro-2H-imidazol-2-one as a white foam (1.59 g, 47.4% yield). ¹H NMR (600 MHz, CDCl₃) δ 9.46 (s, 1H), 7.48 (tq, J=6.6, 2.8, 2.3 Hz, 4H), 7.44-7.40 (m, 2H), 7.30 (dddd, J=12.6, 7.6, 6.3, 4.6 Hz, 1OH), 7.27-7.25 (m, 2H), 7.22-7.18 (m, 1H), 6.85-6.78 (m, 4H), 6.30 (t, J=2.6 Hz, 1H), 6.19 (t, J=2.6 Hz, 1H), 6.03 (dd, J=8.2, 5.8 Hz, 1H), 4.73 (dq, J=9.2, 6.0 Hz, 2H), 3.86 (q, J=3.4 Hz, 1H), 3.76 (s, 6H), 3.49 (ddt, J=14.8, 10.6, 7.6 Hz, 1H), 3.31-3.23 (m, 2H), 3.14 (dd, J=10.3, 3.7 Hz, 1H), 3.04 (tdd, J=10.8, 8.8, 4.3 Hz, 1H), 2.17 (ddd, J=13.4, 5.8, 2.7 Hz, 1H), 2.10 (ddd, J=13.8, 8.3, 6.3 Hz, 1H), 1.83 (ddt, J=12.6, 7.7, 3.9 Hz, 1H), 1.68-1.62 (m, 1H), 1.53 (dd, J=14.6, 8.8 Hz, 1H), 1.43-1.33 (m, 2H), 1.25-1.17 (m, 1H), 0.60 (s, 3H); ³¹P NMR (243 MHz, CDCl₃) δ 151.32; MS (ESI), 842.12 [M+H]⁺.

### Synthesis of (1S,3S,3aS)-1-(((2R,3S,5R)-2-((bis(4-methoxyphenyl)(phenyl)methoxy)methyl)-5-(6-(2-(trimethylsilyl)ethoxy)-9H-purin-9-yl)tetrahydrofuran-3-yl)oxy)-3-((methyldiphenylsilyl)methyl)tetrahydro-1H,3H-pyrrolo[1,2-c][1,3,2]oxazaphosphole

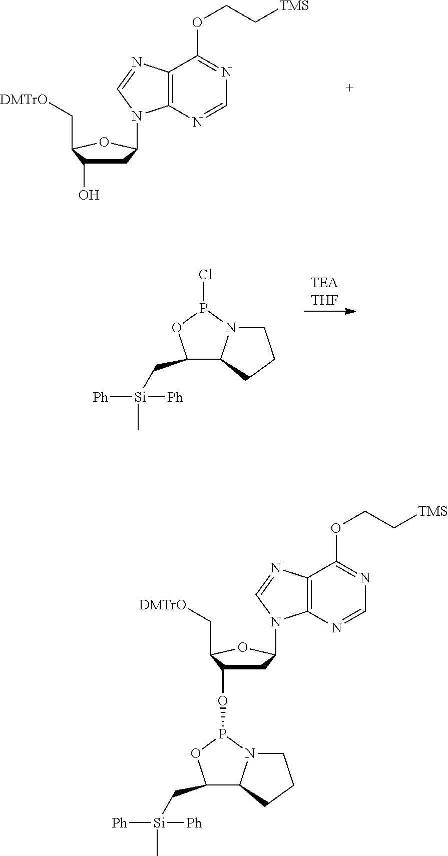

Dry (2R,3R,5R)-2-[[bis(4-methoxyphenyl)-phenyl-methoxy]methyl]-5-[6-(2-trimethylsilylethoxy)purin-9-yl]tetrahydrofuran-3-ol (2.5 g, 3.82 mmol) in a rbf was dissolved in THF (26 mL). To the clear solution was added triethylamine (3.59 mL, 25.77 mmol). [(3S,3aS)-1-chloro-3a,4,5,6-tetrahydro-3H-pyrrolo[1,2-c][1,3,2]oxazaphosphol-3-yl]methyl-methyl-diphenyl-silane (0.96M solution in THF, 7.18 mL, 6.87 mmol) was added dropwise. The resulting cloudy reaction solution was stirred at rt for 4 hr. TLC and LCMS showed the reaction was complete. The mixture was filtered, and the filtrate was concentrated. The resulting crude product was purified by normal phase column chromatography applying 10-85% EtOAc in hexanes (each mobile phase contained 1.5% triethylamine) as the gradient to afford (1S,3S,3aS)-1-(((2R,3S,5R)-2-((bis(4-methoxyphenyl)(phenyl)methoxy)methyl)-5-(6-(2-(trimethylsilyl)ethoxy)-9H-purin-9-yl)tetrahydrofuran-3-yl)oxy)-3-((methyldiphenylsilyl)methyl)tetrahydro-1H,3H-pyrrolo[1,2-c][1,3,2]oxazaphosphole as a white foam (2.89 g, 76.1% yield). ¹H NMR (600 MHz, CDCl₃) δ 8.42 (s, 1H), 7.99 (s, 1H), 7.50-7.45 (m, 4H), 7.41-7.37 (m, 2H), 7.27 (td, J=5.7, 4.9, 1.7 Hz, 7H), 7.25-7.21 (m, 2H), 7.21-7.14 (m, 4H), 6.80-6.74 (m, 4H), 6.34 (dd, J=7.8, 5.9 Hz, 1H), 4.88 (ddt, J=8.8, 5.7, 2.7 Hz, 1H), 4.79 (dt, J=9.1, 5.5 Hz, 1H), 4.73-4.67 (m, 2H), 4.04 (td, J=4.2, 2.5 Hz, 1H), 3.76 (s, 6H), 3.53 (ddt, J=14.8, 10.6, 7.7 Hz, 1H), 3.41-3.33 (m, 1H), 3.29 (dd, J=10.3, 4.3 Hz, 1H), 3.25 (dd, J=10.3, 4.4 Hz, 1H), 3.07 (tdd, J=10.8, 8.9, 4.2 Hz, 1H), 2.54 (ddd, J=13.8, 7.9, 6.1 Hz, 1H), 2.31 (ddd, J=13.5, 6.0, 2.7 Hz, 1H), 1.86 (tt, J=8.8, 4.5 Hz, 1H), 1.74-1.65 (m, 1H), 1.55 (dd, J=14.7, 9.1 Hz, 1H), 1.48-1.39 (m, 2H), 1.33-1.21 (m, 3H), 0.63 (s, 3H), 0.11 (s, 9H); ³¹P NMR (243 MHz, CDCl₃) δ 150.68; MS (ESI), 994.13 [M+H]⁺.

### Synthesis of (1S,3S,3aS)-1-(((2R,3S,5R)-2-((bis(4-methoxyphenyl)(phenyl)methoxy)methyl)-5-(6-(2-(trimethylsilyl)ethoxy)-9H-purin-9-yl)tetrahydrofuran-3-yl)oxy)-3-((phenylsulfonyl)methyl)tetrahydro-1H,3H-pyrrolo[1,2-c][1,3,2]oxazaphosphole

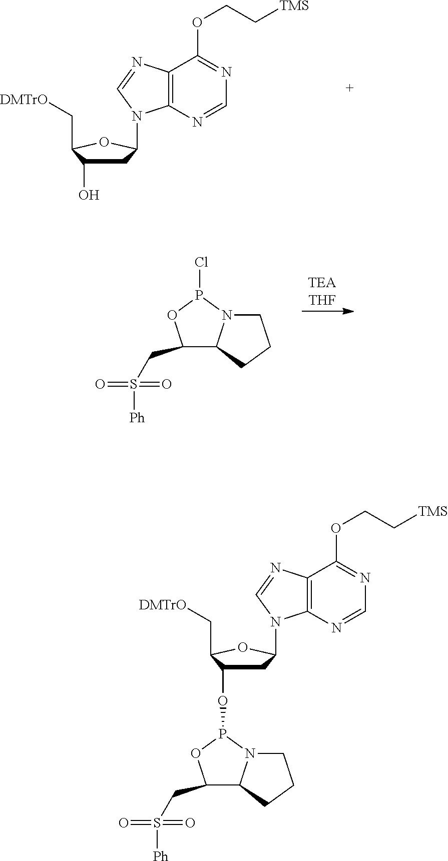

Dry (2R,3R,5R)-2-[[bis(4-methoxyphenyl)-phenyl-methoxy]methyl]-5-[6-(2-trimethylsilylethoxy)purin-9-yl]tetrahydrofuran-3-ol (2.5 g, 3.82 mmol) in a rbf was dissolved in THF (26 mL). To the clear solution was added triethylamine (3.59 mL, 25.77 mmol). (3S,3aS)-3-(benzenesulfonylmethyl)-1-chloro-3a,4,5,6-tetrahydro-3H-pyrrolo[1,2-c][1,3,2]oxazaphosphole (L-PSM C1) (0.9M solution in THF, 7.64 mL, 6.87 mmol) was added dropwise. The resulting cloudy reaction solution was stirred at rt for 4 hr 40 min. TLC and LCMS showed the reaction was complete. The mixture was filtered, and the filtrate was concentrated. The resulting crude product was purified by normal phase column chromatography applying 20-100% EtOAc in hexanes (each mobile phase contained 5% triethylamine) as the gradient to afford (1S,3S,3aS)-1-(((2R,3S,5R)-2-((bis(4-methoxyphenyl)(phenyl)methoxy)methyl)-5-(6-(2-(trimethylsilyl)ethoxy)-9H-purin-9-yl)tetrahydrofuran-3-yl)oxy)-3-((phenylsulfonyl)methyl)tetrahydro-1H,3H-pyrrolo[1,2-c][1,3,2]oxazaphosphole as a white foam (1.51 g, 42.3% yield). ¹H NMR (600 MHz, CDCl₃) δ 8.44 (s, 1H), 8.05 (s, 1H), 7.92-7.87 (m, 2H), 7.60-7.54 (m, 1H), 7.48 (t, J=7.8 Hz, 2H), 7.43-7.37 (m, 2H), 7.31-7.28 (m, 3H), 7.26-7.23 (m, 3H), 7.22-7.16 (m, 1H), 6.79 (d, J=8.7 Hz, 4H), 6.44 (dd, J=7.8, 5.9 Hz, 1H), 5.04 (q, J=6.0 Hz, 1H), 4.93 (ddt, J=8.8, 5.8, 2.6 Hz, 1H), 4.72-4.66 (m, 2H), 4.17 (td, J=4.4, 2.4 Hz, 1H), 3.77 (s, 6H), 3.66 (dq, J=10.1, 6.0 Hz, 1H), 3.56-3.50 (m, 1H), 3.48 (dd, J=14.8, 7.1 Hz, 1H), 3.37 (dt, J=14.9, 5.1 Hz, 2H), 3.31 (dd, J=10.3, 4.5 Hz, 1H), 3.18-3.09 (m, 1H), 2.89 (ddd, J=13.7, 7.9, 6.0 Hz, 1H), 2.56 (ddd, J=13.6, 6.0, 2.6 Hz, 1H), 1.93-1.86 (m, 1H), 1.79 (dt, J=11.6, 8.6 Hz, 1H), 1.69-1.64 (m, 1H), 1.31-1.25 (m, 2H), 1.18-1.08 (m, 1H), 0.10 (s, 9H); ³¹P NMR (243 MHz, CDCl₃) δ 152.58; MS (ESI), 938.58 [M+H]⁺.

### Synthesis of N-(9-((2R,4S,5R)-5-((bis(4-methoxyphenyl)(phenyl)methoxy)methyl)-4-hydroxytetrahydrofuran-2-yl)-8-oxo-8,9-dihydro-7H-purin-6-yl)benzamide (WV-NU-137)

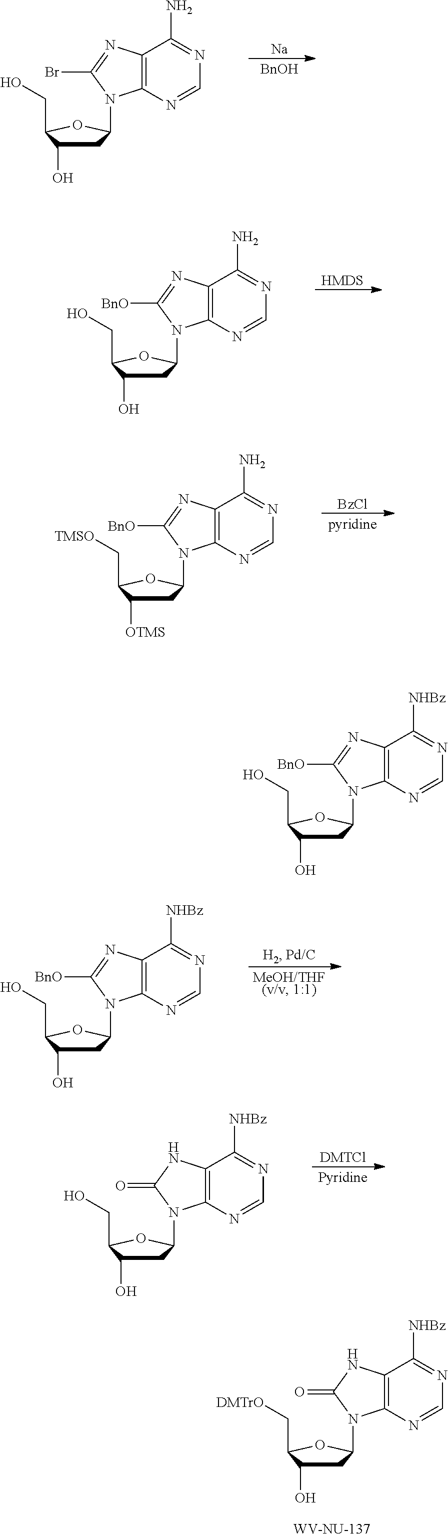

Step 1. A solution of Na (9.99 g, 434.67 mmol) in BnOH (391.84 g, 3.62 mol), 3 hr later, (2R,3S,5R)-5-(6-amino-8-bromo-9H-purin-9-yl)-2-(hydroxymethyl)tetrahydrofuran-3-ol (25 g, 75.73 mmol) was added. The mixture was stirred at 15° C. for 12 hr. The reaction mixture was quenched by addition HCl (1M) 800 mL at 0° C., then added sat. NaHCO₃ aq. until pH-9, and extracted with EtOAc (1000 mL*3), dried over Na₂SO₄, filtered and concentrated under reduced pressure to give a residue. The residue was purified by column chromatography ((SiO₂, Petroleum ether/Ethyl acetate=5/1 to Ethyl acetate Methanol=10/1) to get (2R,3S,5R)-5-(6-amino-8-(benzyloxy)-9H-purin-9-yl)-2-(hydroxymethyl)tetrahydrofuran-3-ol (35 g, 64.67% yield) was obtained as a yellow oil. LCMS: (M+H⁺): 358.2.

Step 2. (2R,3S,5R)-5-(6-amino-8-(benzyloxy)-9H-purin-9-yl)-2-(hydroxymethyl)tetrahydrofuran-3-ol (50 g, 139.91 mmol) (dried by azeotropic distillation on a rotary evaporator with pyridine (200 mL*3)) was added HMDS (338.72 g, 2.10 mol). The mixture was stirred at 150° C. for 12 hr. The reaction mixture was concentrated under reduced pressure to remove solvent. 8-(benzyloxy)-9-((2R,4S,5R)-4-((trimethylsilyl)oxy)-5-(((trimethylsilyl)oxy)methyl)tetrahydrofuran-2-yl)-9H-purin-6-amine (70.2 g, crude) was obtained as a yellow oil without purification.

Step 3. To a solution of 8-(benzyloxy)-9-((2R,4S,5R)-4-((trimethylsilyl)oxy)-5-(((trimethylsilyl)oxy)methyl)tetrahydrofuran-2-yl)-9H-purin-6-amine (70.2 g) in pyridine (500 mL) was added BzCl (29.50 g). The mixture was stirred at 20° C. for 2 hr. MeOH (500 mL) and water (500 mL) was added, 10 min later NH₃·H₂O (250 mL) was added, 30 min later H₂O (500 mL) was added and extracted with EtOAc (500 mL*4). dried over Na₂SO₄, filtered and concentrated under reduced pressure to give a residue. The residue was purified by column chromatography (SiO₂, Petroleum ether/Ethyl acetate=I/O to 0/1, then ethyl acetate/methanol=10:1) to get N-(8-(benzyloxy)-9-((2R,4S,5R)-4-hydroxy-5-(hydroxymethyl)tetrahydrofuran-2-yl)-9H-purin-6-yl)benzamide (36 g, 55.76% yield) as a yellow solid. LCMS: (M+H+): 462.2.

Step 4. To a solution of N-(8-(benzyloxy)-9-((2R,4S,5R)-4-hydroxy-5-(hydroxymethyl)tetrahydrofuran-2-yl)-9H-purin-6-yl)benzamide (36 g, 78 mmol) in THF (500 mL) and MeOH (500 mL) was added Pd/C (9 g, 39.01 mmol, 10% purity). The mixture was stirred at 15° C. for 3 hr in H₂ (15 psi). The mixture was filtered, and the filtrated was concentrated under the reduced pressure to get N-(9-((2R,4S,5R)-4-hydroxy-5-(hydroxymethyl)tetrahydrofuran-2-yl)-8-oxo-8,9-dihydro-7H-purin-6-yl)benzamide (28.9 g, crude) as a yellow solid. LCMS: (M+H+): 372.2.

Step 5. To a solution of N-(9-((2R,4S,5R)-4-hydroxy-5-(hydroxymethyl)tetrahydrofuran-2-yl)-8-oxo-8,9-dihydro-7H-purin-6-yl)benzamide (28.9 g, 77.82 mmol) in pyridine (300 mL) was added DMTCl (26.37 g, 77.82 mmol), the mixture was stirred at 15° C. for 12 hr. The reaction mixture was quenched by addition water (200 mL) at 0° C., and extracted with EtOAc (300 mL*3). Dried over Na₂SO₄, filtered and concentrated under reduced pressure to give a residue. The residue was purified by silica gel chromatography (Petroleum ether/Ethyl acetate=10/1, 1/4, 5% TEA) to get N-(9-((2R,4S,5R)-5-((bis(4-methoxyphenyl)(phenyl)methoxy)methyl)-4-hydroxytetrahydrofuran-2-yl)-8-oxo-8,9-dihydro-7H-purin-6-yl)benzamide (WV-NU-137) (32 g, 57.75% yield) as a white solid. ¹H NMR (400 MHz, 400 MHz, DMSO-d6) δ=8.38-8.24 (m, 1H), 8.12-8.00 (m, 2H), 7.67-7.60 (m, 1H), 7.58-7.51 (m, 2H), 7.38-7.33 (m, 2H), 7.26-7.13 (m, 7H), 6.81 (dd, J=9.0, 13.3 Hz, 4H), 6.25 (t, J=6.8 Hz, 1H), 5.29 (d, J=4.6 Hz, 1H), 4.56-4.49 (m, 1H), 3.95 (q, J=4.9 Hz, 1H), 3.71 (d, J=4.4 Hz, 6H), 3.20-3.15 (m, 2H), 3.08 (td, J=6.5, 13.0 Hz, 1H), 2.21-2.10 (m, 1H); LCMS (M−H⁺): 672.2.

### Synthesis of N-(9-((2R,4S,5R)-5-((bis(4-methoxyphenyl)(phenyl)methoxy)methyl)-4-(((1S,3S,3aS)-3-((methyldiphenylsilyl)methyl)tetrahydro-1H,3H-pyrrolo[1,2-c][1,3,2]oxazaphosphol-1-yl)oxy)tetrahydrofuran-2-yl)-8-oxo-8,9-dihydro-7H-purin-6-yl)benzamide

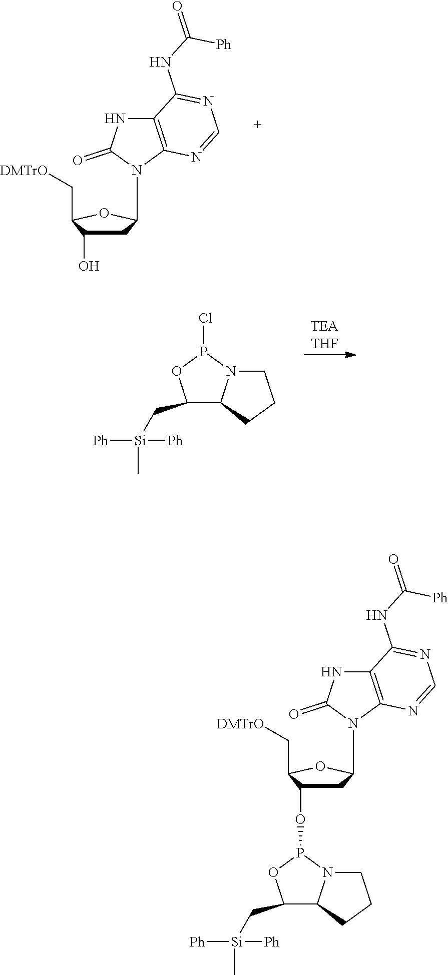

Dry N-[9-[(2R,4R,5R)-5-[[bis(4-methoxyphenyl)-phenyl-methoxy]methyl]-4-hydroxy-tetrahydrofuran-2-yl]-8-oxo-7H-purin-6-yl]benzamide (4.0 g, 5.94 mmol) in a rbf was dissolved in THF (50 mL). To the clear solution was added triethylamine (5.59 mL, 40.08 mmol). [(3S,3aS)-1-chloro-3a,4,5,6-tetrahydro-3H-pyrrolo[1,2-c][1,3,2]oxazaphosphol-3-yl]methyl-methyl-diphenyl-silane (0.96M solution in THF, 11.16 mL, 10.69 mmol) was added dropwise. The reaction solution was stirred at rt for 2 hr. TLC showed the reaction was complete. Anhydrous MgSO4 (708 mg) was added. Stirred for 1 min. The mixture was filtered, and the filtrate was concentrated. The resulting crude product was purified by normal phase column chromatography applying 0-100% EtOAc in hexanes (each mobile phase contained 1.5% triethylamine) as the gradient to afford N-(9-((2R,4S,5R)-5-((bis(4-methoxyphenyl)(phenyl)methoxy)methyl)-4-(((1S,3S,3aS)-3-((methyldiphenylsilyl)methyl)tetrahydro-1H,3H-pyrrolo[1,2-c][1,3,2]oxazaphosphol-1-yl)oxy)tetrahydrofuran-2-yl)-8-oxo-8,9-dihydro-7H-purin-6-yl)benzamide as a white foam (4.45 g, 74.0% yield). ¹H NMR (600 MHz, CDCl₃) δ 9.42 (s, 1H), 8.59 (s, 1H), 8.17 (s, 1H), 7.98-7.93 (m, 2H), 7.68-7.62 (m, 1H), 7.58-7.53 (m, 2H), 7.53-7.46 (m, 4H), 7.45-7.40 (m, 2H), 7.33-7.26 (m, 7H), 7.24-7.17 (m, 5H), 7.16-7.11 (m, 1H), 6.76-6.69 (m, 4H), 6.30 (dd, J=7.3, 6.1 Hz, 1H), 5.05 (ddt, J=8.9, 6.9, 4.5 Hz, 1H), 4.85 (dt, J=8.9, 5.7 Hz, 1H), 4.03 (q, J=5.0 Hz, 1H), 3.73 (d, J=4.5 Hz, 6H), 3.49 (ddt, J=14.6, 10.6, 7.6 Hz, 1H), 3.40 (ddt, J=12.6, 7.0, 5.5 Hz, 1H), 3.34 (dd, J=10.1, 4.9 Hz, 1H), 3.25 (dd, J=10.1, 5.9 Hz, 1H), 2.97 (tdd, J=10.8, 8.8, 4.3 Hz, 1H), 2.83 (dt, J=13.3, 6.6 Hz, 1H), 2.08 (ddd, J=13.5, 7.4, 4.6 Hz, 1H), 1.84 (ddt, J=12.2, 8.5, 4.3 Hz, 1H), 1.70-1.63 (m, 1H), 1.55 (dd, J=14.7, 8.9 Hz, 1H), 1.45-1.38 (m, 2H), 1.30-1.20 (m, 1H), 0.65 (s, 3H); ³¹P NMR (243 MHz, CDCl₃) δ 148.40; MS (ESI), 1013.18 [M+H]⁺.

### Synthesis of N-(9-((2R,4S,5R)-5-((bis(4-methoxyphenyl)(phenyl)methoxy)methyl)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-8-oxo-8,9-dihydro-7H-purin-6-yl)benzamide

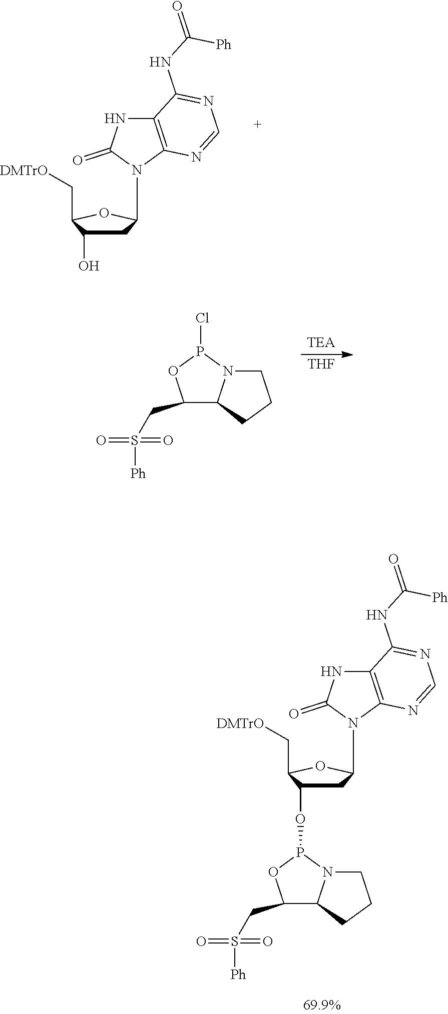

To a solution of dry N-[9-[(2R,4R,5R)-5-[[bis(4-methoxyphenyl)-phenyl-methoxy]methyl]-4-hydroxy-tetrahydrofuran-2-yl]-8-oxo-7H-purin-6-yl]benzamide (3.0 g, 4.45 mmol) in THF (30 mL) was added triethylamine (1.55 mL, 11.13 mmol). (3S,3aS)-3-(benzenesulfonylmethyl)-1-chloro-3a,4,5,6-tetrahydro-3H-pyrrolo[1,2-c][1,3,2]oxazaphosphole (0.9M in THF, 8.91 mL, 8.02 mmol) was added dropwise. The resulting off-white slurry was stirred at rt for 2 hr. TLC and LCMS showed the reaction was complete. The reaction was quenched by water (80 uL). Anhydrous MgSO4 (1.07 g) was added. The mixture was filtered through celite, and the filtrate was concentrated to afford the crude product as an off-white foam. The crude product was purified by normal phase column chromatography applying 20-100% EtOAc in hexanes (each mobile phase contained 2.5% triethylamine) as the gradient to afford the title compound as a white foam (2.979 g, 69.9% yield). ¹H NMR (600 MHz, CDCl₃) δ 9.45 (s, 1H), 8.60 (s, 1H), 8.24 (s, 1H), 7.97-7.92 (m, 2H), 7.92-7.88 (m, 2H), 7.67-7.62 (m, 1H), 7.62-7.57 (m, 1H), 7.57-7.48 (m, 4H), 7.45-7.40 (m, 2H), 7.34-7.28 (m, 4H), 7.21 (dd, J=8.3, 6.7 Hz, 2H), 7.19-7.13 (m, 1H), 6.79-6.72 (m, 4H), 6.39 (t, J=6.8 Hz, 1H), 5.09 (ddt, J=14.7, 6.9, 4.9 Hz, 2H), 4.08-4.03 (m, 1H), 3.76 (s, 3H), 3.75 (s, 3H), 3.69 (dq, J=9.8, 5.9 Hz, 1H), 3.52-3.42 (m, 2H), 3.37 (ddd, J=12.2, 5.4, 2.4 Hz, 2H), 3.34-3.24 (m, 2H), 3.03 (tdd, J=10.3, 8.8, 4.1 Hz, 1H), 2.30 (ddd, J=13.5, 7.3, 4.5 Hz, 1H), 1.87 (dt, J=11.4, 5.9 Hz, 1H), 1.80-1.72 (m, 1H), 1.70-1.63 (m, 1H), 1.12 (dtd, J=11.7, 10.1, 8.5 Hz, 1H); ³¹P NMR (243 MHz, CDCl₃) δ 149.85; MS (ESI), 955.37 [M−H]⁻.

### Synthesis of N-(9-((2R,3R,4R,5R)-5-((bis(4-methoxyphenyl)(phenyl)methoxy)methyl)-3-((tert-butyldimethylsilyl)oxy)-4-hydroxytetrahydrofuran-2-yl)-8-oxo-8,9-dihydro-7H-purin-6-yl)benzamide

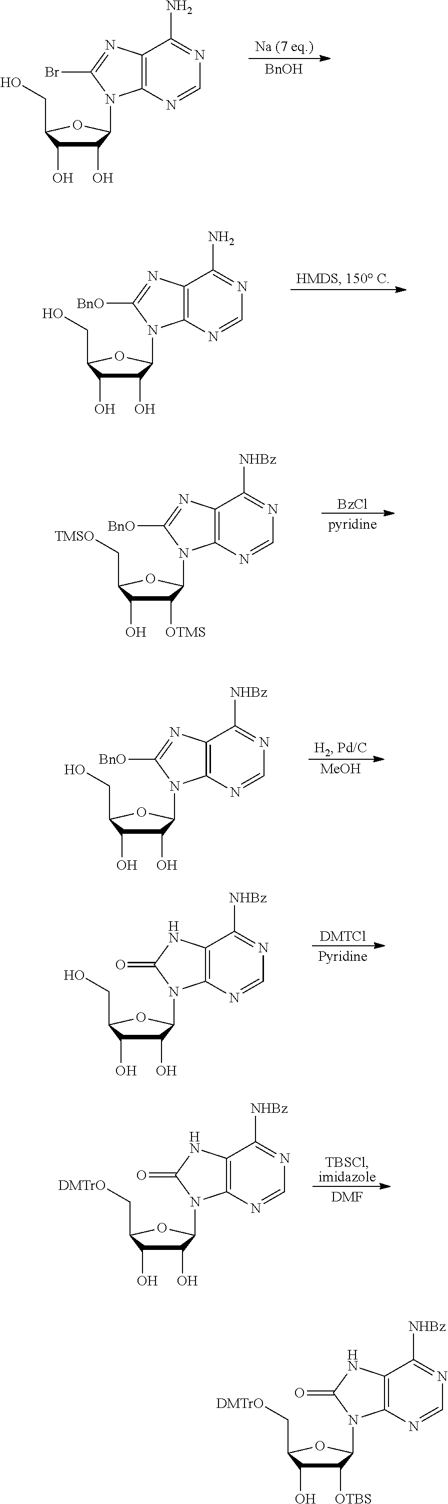

Step 1. To a solution of Na (21 g, 913.45 mmol, 21.65 mL, 8.43 eq.) in BnOH (1000 mL), 3 hr later, (2R,3R,4S,5R)-2-(6-amino-8-bromo-9H-purin-9-yl)-5-(hydroxymethyl)tetrahydrofuran-3,4-diol (37.5 g, 108.34 mmol, 1.0 eq.) was added. The mixture was stirred at 15° C. for 12 hr. The mixture was poured into cold 1N HCl (2500 mL) and extracted with EtOAc (1500 mL). The aqueous phase was added sat.NaHCO₃ (aq) until pH >8, and the white cake was separated out, filtered and concentrated to get the crude. (2R,3R,4S,5R)-2-(6-amino-8-(benzyloxy)-9H-purin-9-yl)-5-(hydroxymethyl)tetrahydrofuran-3,4-diol (80 g, crude) was obtained as white solid. LCMS: (M+H⁺): 374.4.

Step 2. To a solution of (2R,3R,4S,5R)-2-(6-amino-8-(benzyloxy)-9H-purin-9-yl)-5-(hydroxymethyl)tetrahydrofuran-3,4-diol (39.0 g, 104.46 mmol, 1.0 eq.) in HMDS (400 mL), the mixture was stirred at 130° C. for 12 hrs. The reaction mixture was concentrated under reduced pressure to give a residue. The N-(8-(benzyloxy)-9-((2R,3R,4R,5R)-4-hydroxy-3-((trimethylsilyl)oxy)-5-(((trimethylsilyl)oxy)methyl)tetrahydrofuran-2-yl)-9H-purin-6-yl)benzamide (61.62 g, crude) was obtained as brown solid.

Step 3. To a solution of N-(8-(benzyloxy)-9-((2R,3R,4R,5R)-4-hydroxy-3-((trimethylsilyl)oxy)-5-(((trimethylsilyl)oxy)methyl)tetrahydrofuran-2-yl)-9H-purin-6-yl)benzamide (46.0 g, 77.98 mmol, 1 eq.) in pyridine (460 mL) was added benzoyl chloride (21.92 g, 155.96 mmol, 18.12 mL, 2.0 eq.). The mixture was stirred at 20° C. for 1 hr. The reaction mixture was added MeOH:H₂O (1:1) 500 mL and stirred at 15° C. for 10 mins. Then the mixture was added NH₃. H₂O (150 mL) and stirred for 10 min at 15° C. Then the mixture was diluted by H₂O 200 mL and exacted by EtOAc 800 mL (200 mL*4). The mixture was added brine 200 mL and dried over Na₂SO₄. Then the mixture was concentrated under reduced pressure to give a residue. The residue was purified by column chromatography. N-(8-(benzyloxy)-9-((2R,3R,4S,5R)-3,4-dihydroxy-5-(hydroxymethyl)tetrahydrofuran-2-yl)-9H-purin-6-yl)benzamide (33.99 g, 71.19 mmol, 91.29% yield) was obtained as yellow solid. LCMS: (M+H⁺): 478.4.

Step 4. To a solution of N-(8-(benzyloxy)-9-((2R,3R,4S,5R)-3,4-dihydroxy-5-(hydroxymethyl)tetrahydrofuran-2-yl)-9H-purin-6-yl)benzamide (35.1 g, 73.51 mmol, 1 eq.) in MeOH (1500 mL) and THF (500 mL) was added Pd/C (7.0 g, 10% purity) under H₂ (15 psi). The mixture was stirred at 20° C. for 1 hr. The reaction was filtered and concentrated under reduced pressure to give a residue. The residue was not purified and the N-(9-((2R,3R,4S,5R)-3,4-dihydroxy-5-(hydroxymethyl)tetrahydrofuran-2-yl)-8-oxo-8,9-dihydro-7H-purin-6-yl)benzamide (19.6 g, 50.60 mmol, 68.83% yield) was obtained as brown solid. LCMS: (M+H⁺): 388.2.

Step 5. To a solution of N-(9-((2R,3R,4S,5R)-3,4-dihydroxy-5-(hydroxymethyl)tetrahydrofuran-2-yl)-8-oxo-8,9-dihydro-7H-purin-6-yl)benzamide (14.8 g, 38.21 mmol, 1 eq.) in pyridine (150 mL) was added DMTCl (15.54 g, 45.85 mmol, 1.2 eq.). The mixture was stirred at 20° C. for 2 hrs. The reaction mixture was diluted with H₂O 10 mL and extracted with ethyl acetate. The combined organic layers were washed with brine 100 mL, dried over Na₂SO₄, filtered and concentrated under reduced pressure to give a residue. Residue was purified by column chromatography (Petroleum ether/Ethyl acetate=100/1 to 0/1). N-(9-((2R,3R,4S,5R)-5-((bis(4-methoxyphenyl)(phenyl)methoxy)methyl)-3,4-dihydroxytetrahydrofuran-2-yl)-8-oxo-8,9-dihydro-7H-purin-6-yl)benzamide (13.2 g, 19.14 mmol, 50.09% yield) was obtained as brown solid. LCMS: (M+H⁺): 690.5.

Step 6. To a solution of N-(9-((2R,3R,4S,5R)-5-((bis(4-methoxyphenyl)(phenyl)methoxy)methyl)-3,4-dihydroxytetrahydrofuran-2-yl)-8-oxo-8,9-dihydro-7H-purin-6-yl)benzamide (10.20 g, 14.79 mmol, 1 eq.) in DMF (100 mL) was added imidazole (3.02 g, 44.37 mmol, 3.00 eq.) and TBSCl (2.01 g, 13.31 mmol, 1.63 mL, 0.9 eq.). The mixture was stirred at 15° C. for 10 hrs. The mixture was diluted with ethyl acetate and washed with NaHCO₃ solution. The combined organic layers were dried over Na₂SO₄, filtered and concentrated under reduced pressure to give a residue. The residue was purified by column chromatography (Petroleum ether/Ethyl acetate=100/1 to 1/1). N-(9-((2R,3R,4R,5R)-5-((bis(4-methoxyphenyl)(phenyl)methoxy)methyl)-3-((tert-butyldimethylsilyl)oxy)-4-hydroxytetrahydrofuran-2-yl)-8-oxo-8,9-dihydro-7H-purin-6-yl)benzamide (3.82 g, 4.75 mmol, 32.13% yield) was obtained as yellow solid. ¹HNMR (400 MHz, CHLOROFORM-d) 6=9.51 (s, 1H), 8.57 (s, 1H), 8.26 (s, 1H), 8.03 (s, 1H), 7.96 (d, J=7.5 Hz, 2H), 7.70-7.63 (m, 1H), 7.61-7.54 (m, 2H), 7.48 (d, J=7.3 Hz, 2H), 7.36 (dd, J=2.0, 8.9 Hz, 4H), 7.26-7.16 (m, 3H), 6.78 (d, J=8.7 Hz, 4H), 5.99 (d, J=4.6 Hz, 1H), 5.32-5.27 (m, 1H), 4.48 (q, J=5.5 Hz, 1H), 4.13-4.08 (m, 1H), 3.78 (s, 6H), 3.46 (dd, J=3.9, 10.3 Hz, 1H), 3.32 (dd, J=5.3, 10.3 Hz, 1H), 2.70 (d, J=5.9 Hz, 1H), 2.06 (s, 1H), 1.58 (s, 2H), 1.27 (t, J=7.2 Hz, 1H), 0.89 (s, 9H), 0.05 (s, 3H),−0.01 (s, 3H); LCMS: (M−H⁺): 802.3.

### Synthesis of N-(9-((2R,3R,4R,5R)-5-((bis(4-methoxyphenyl)(phenyl)methoxy)methyl)-3-((tert-butyldimethylsilyl)oxy)-4-(((1S,3S,3aS)-3-((phenylsulfonyl)methyl)tetrahydro-1H,3H-pyrrolo[1,2-c][1,3,2]oxazaphosphol-1-yl)oxy)tetrahydrofuran-2-yl)-8-oxo-8,9-dihydro-7H-purin-6-yl)benzamide

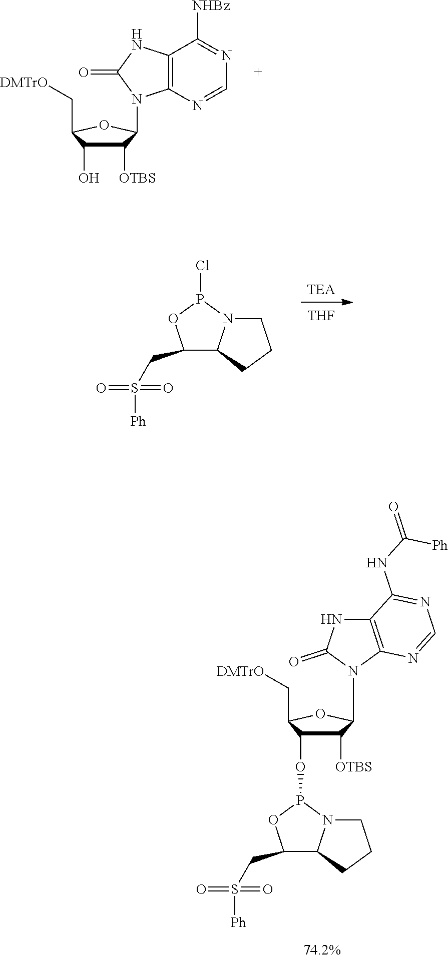

To a solution of dry N-[9-[(2R,3S,5R)-5-[[bis(4-methoxyphenyl)-phenyl-methoxy]methyl]-3-[tert-butyl(dimethyl)silyl]oxy-4-hydroxy-tetrahydrofuran-2-yl]-8-oxo-7H-purin-6-yl]benzamide (3.5 g, 4.35 mmol) in THF (35 mL) was added triethylamine (1.52 mL, 10.88 mmol). (3S,3aS)-3-(benzenesulfonylmethyl)-1-chloro-3a,4,5,6-tetrahydro-3H-pyrrolo[1,2-c][1,3,2]oxazaphosphole (0.9M in THF, 8.71 mL, 7.84 mmol) was added dropwise. The resulting cloudy solution was stirred at rt for 3.5 hr. TLC and LCMS showed the reaction was complete. The reaction was quenched by water (78 uL). Anhydrous MgSO₄ (1.05 g) was added. The mixture was filtered through celite, and the filtrate was concentrated to afford the crude product as an off-white foam. The crude product was purified by normal phase column chromatography applying 20-100% EtOAc in hexanes (each mobile phase contained 2.5% triethylamine) as the gradient to afford the title compound as a white foam (3.512 g, 74.2% yield). ¹H NMR (600 MHz, CDCl₃) δ 9.48 (s, 1H), 8.62 (s, 1H), 8.24 (s, 1H), 7.96-7.92 (m, 2H), 7.90-7.85 (m, 2H), 7.67-7.61 (m, 1H), 7.57 (td, J=7.2, 1.2 Hz, 1H), 7.54 (t, J=7.8 Hz, 2H), 7.50-7.43 (m, 4H), 7.38-7.32 (m, 4H), 7.22 (dd, J=8.4, 6.9 Hz, 2H), 7.19-7.13 (m, 1H), 6.79-6.72 (m, 4H), 6.01 (d, J=5.4 Hz, 1H), 5.33 (t, J=5.3 Hz, 1H), 5.00 (q, J=6.2 Hz, 1H), 4.78 (dt, J=10.8, 4.7 Hz, 1H), 4.06 (q, J=4.4 Hz, 1H), 3.76 (s, 6H), 3.67 (dq, J=11.4, 5.8 Hz, 1H), 3.49-3.34 (m, 4H), 3.19 (dd, J=10.4, 4.9 Hz, 1H), 3.01 (qd, J=9.5, 4.0 Hz, 1H), 1.85 (t, J=5.8 Hz, 1H), 1.77-1.70 (m, 1H), 1.68-1.62 (m, 1H), 1.16-1.06 (m, 1H), 0.83 (s, 9H), 0.02 (s, 3H),−0.09 (s, 3H); ³¹P NMR (243 MHz, CDCl₃) δ 152.12; MS (ESI), 1086.13 [M−H]⁻.

### Synthesis of 1-((2R,4S,5R)-5-((bis(4-methoxyphenyl)(phenyl)methoxy)methyl)-4-hydroxytetrahydrofuran-2-yl)-1,3-dihydro-2H-benzo[d]imidazol-2-one

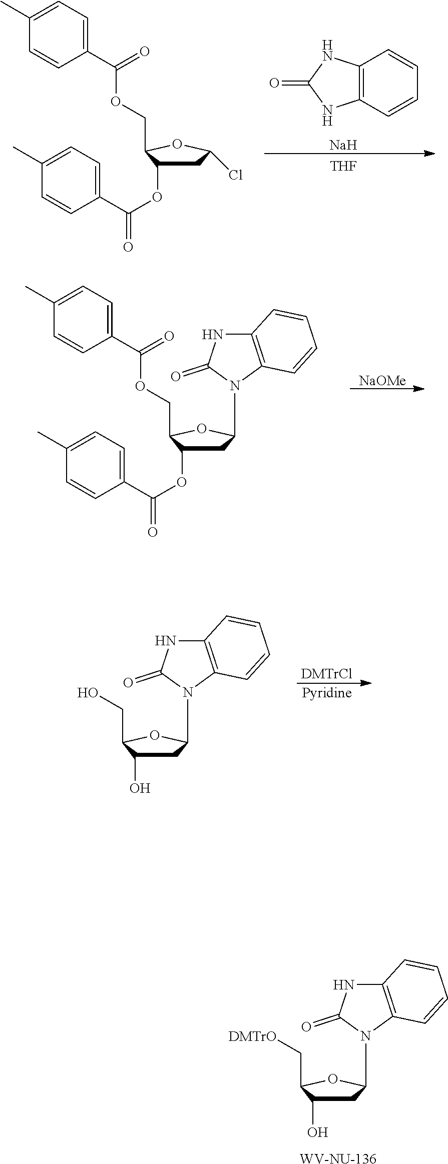

Step 1. To a solution of 1,3-dihydrobenzimidazol-2-one (30 g, 223.66 mmol, 1 eq.) in THF (210 mL) was added NaH (22.37 g, 559.14 mmol, 60% purity, 2.5 eq.) at 0° C. The reaction was stirred for 15 mim then added (2R,3S,5R)-5-chloro-2-(((4-methylbenzoyl)oxy)methyl)tetrahydrofuran-3-yl 4-methylbenzoateto the above reaction mixture at once. The resulting suspension was stirred for 5 h. TLC (Petroleum ether: Ethyl acetate=2: 1) showed the product was detected. The reaction was quenched by the addition of H₂O (2000 mL) and extracted twice with 2000 mL ethyl acetate. The organic layer was washed successively with saturated aqueous NaCl and H₂O and dried with anhydrous Na₂SO₄. The organic solvent was evaporated under reduced pressure. The residue was purified by prep-MPLC (neutral condition) to give ((2R,3S,5R)-3-((4-methylbenzoyl)oxy)-5-(2-oxo-2,3-dihydro-1H-benzo[d]imidazol-1-yl)tetrahydrofuran-2-yl)methyl 4-methylbenzoate (25 g, crude) as a white foamy solid.

Step 2. To a solution of ((2R,3S,5R)-3-((4-methylbenzoyl)oxy)-5-(2-oxo-2,3-dihydro-1H-benzo[d]imidazol-1-yl)tetrahydrofuran-2-yl)methyl 4-methylbenzoate (25 g, 51.39 mmol, 1 eq.) in MeOH (250 mL) was added NaOMe (8.33 g, 154.16 mmol, 3 eq.). The reaction was stirred at 25° C. for 12 h. TLC (Petroleum ether: Ethyl acetate=1: 1) showed the product was detected. Added NH₄Cl (8.33 g) to the above reaction. The reaction mixture was concentrated under reduced pressure to give yellow oil to give 1-((2R,4S,5R)-4-hydroxy-5-(hydroxymethyl)tetrahydrofuran-2-yl)-1,3-dihydro-2H-benzo[d]imidazol-2-one (12.86 g, crude) as yellow oil.

Step 3. To a solution of 1-((2R,4S,5R)-4-hydroxy-5-(hydroxymethyl)tetrahydrofuran-2-yl)-1,3-dihydro-2H-benzo[d]imidazol-2-one (12.86 g, 51.39 mmol, 1 eq.) in Pyridine (60 mL), DMTrCl (19.15 g, 56.53 mmol, 1.1 eq.) was added. The reaction was stirred at 25° C. for 5 h. LCMS (product: RT=1.305 min) showed the product was detected. The combined extracts were washed with H₂O (500 mL) and brine (450 mL). The solution was dried (Na₂SO₄), filtered, and concentrated at reduced pressure. The residue was purified by column chromatography (SiO₂, Petroleum ether/Ethyl acetate=10/1 to 0/1) to give product 1-((2R,4S,5R)-5-((bis(4-methoxyphenyl)(phenyl)methoxy)methyl)-4-hydroxytetrahydrofuran-2-yl)-1,3-dihydro-2H-benzo[d]imidazol-2-one (WV-NU-136) (10.20 g, 18.26 mmol, 35.54% yield, 98.95% purity) as yellow solid. ¹H NMR (400 MHz, DMSO-d₆) δ=10.93 (s, 1H), 7.37 (d, J=7.1 Hz, 2H), 7.26-7.21 (m, 7H), 6.97 (d, J=4.0 Hz, 2H), 6.81 (t, J=8.3 Hz, 4H), 6.72-6.65 (m, 1H), 6.72-6.65 (m, 1H), 6.14 (t, J=7.1 Hz, 1H), 5.30 (d, J=4.9 Hz, 1H), 4.43 (br dd, J=4.0, 7.3 Hz, 1H), 3.87 (br d, J=3.1 Hz, 1H), 3.70 (d, J=1.3 Hz, 6H), 3.27-3.20 (m, 1H), 3.17-3.13 (m, 1H), 2.75-2.64 (m, 1H), 2.01 (ddd, J=3.4, 6.7, 10.0 Hz, 1H); ¹³C NMR (101 MHz, DMSO-d₆) δ=177.55, 163.23, 158.43, 150.01, 140.76, 135.02, 134.93, 133.64, 133.26, 132.97, 131.85, 126.59, 125.56, 118.35, 115.20, 118.34, 115.20, 114.13, 90.71, 89.86, 86.72, 75.60, 68.98, 64.97, 60.23, 45.37, 45.16, 44.95, 44.54, 44.74 (br t, J=41.8 Hz, 1C), 44.12, 25.98, 19.30; LCMS: M+H⁺=551.2; purity: 98.95%.

### Synthesis of 1-((2R,4S,5R)-5-((bis(4-methoxyphenyl)(phenyl)methoxy)methyl)-4-(((1S,3S,3aS)-3-((methyldiphenylsilyl)methyl)tetrahydro-1H,3H-pyrrolo[1,2-c][1,3,2]oxazaphosphol-1-yl)oxy)tetrahydrofuran-2-yl)-1,3-dihydro-2H-benzo[d]imidazol-2-one

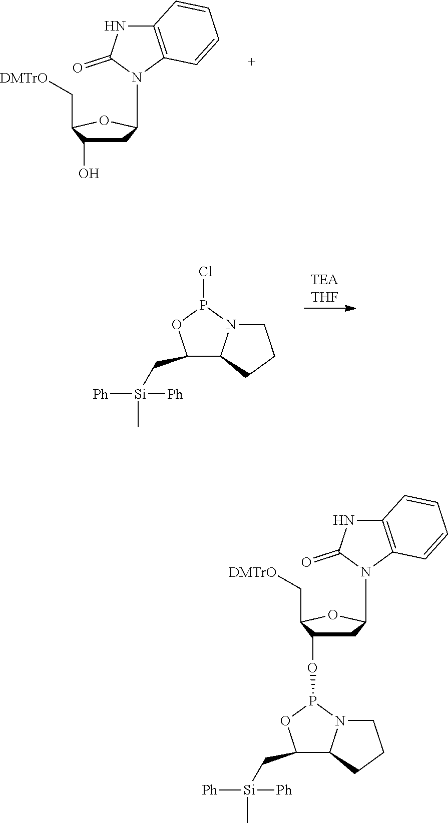

Dry 3-[(2R,4R,5R)-5-[[bis(4-methoxyphenyl)-phenyl-methoxy]methyl]-4-hydroxy-tetrahydrofuran-2-yl]-1H-benzimidazol-2-one (4.0 g, 7.24 mmol) in a rbf was dissolved in THF (50 mL). To the clear solution was added triethylamine (5.67 mL, 40.72 mmol). [(3S,3aS)-1-chloro-3a,4,5,6-tetrahydro-3H-pyrrolo[1,2-c][1,3,2]oxazaphosphol-3-yl]methyl-methyl-diphenyl-silane (0.96M solution in THF, 11.34 mL, 10.86 mmol) was added dropwise. The resulting cloudy reaction solution was stirred at rt for 2 hr. TLC and LCMS showed the reaction was not complete. Additional triethylamine (1.13 mL, 8.143 mmol) was added followed by additional [(3S,3aS)-1-chloro-3a,4,5,6-tetrahydro-3H-pyrrolo[1,2-c][1,3,2]oxazaphosphol-3-yl]methyl-methyl-diphenyl-silane solution (2.27 mL, 2.1714 mmol). Stirred for 2 hr. TLC and LCMS showed the reaction was complete. Triethylamine (5.7 mL) was added followed by Na₂SO₄ (1.54 g). Stirred for 5 min. The mixture was filtered, and the filtrate was concentrated. The resulting crude product was purified by normal phase column chromatography applying 10-75% EtOAc in hexanes (each mobile phase contained 5% triethylamine) as the gradient to afford 1-((2R,4S,5R)-5-((bis(4-methoxyphenyl)(phenyl)methoxy)methyl)-4-(((1S,3S,3aS)-3-((methyldiphenylsilyl)methyl)tetrahydro-1H,3H-pyrrolo[1,2-c][1,3,2]oxazaphosphol-1-yl)oxy)tetrahydrofuran-2-yl)-1,3-dihydro-2H-benzo[d]imidazol-2-one as a white foam (4.89 g, 75.7% yield). ¹H NMR (600 MHz, CDCl₃) δ 9.10 (s, 1H), 7.51-7.43 (m, 6H), 7.38-7.30 (m, 5H), 7.29-7.16 (m, 8H), 7.16-7.10 (m, 1H), 7.02-6.96 (m, 2H), 6.79-6.75 (m, 4H), 6.58 (td, J=7.5, 1.6 Hz, 1H), 6.21 (dd, J=8.7, 6.1 Hz, 1H), 4.99 (td, J=7.6, 6.6, 3.3 Hz, 1H), 4.78 (dt, J=8.8, 5.7 Hz, 1H), 3.85 (p, J=3.7 Hz, 1H), 3.74 (s, 6H), 3.51 (ddt, J=14.8, 10.5, 7.7 Hz, 1H), 3.37-3.29 (m, 3H), 3.03 (qd, J=10.6, 4.1 Hz, 1H), 2.56 (dt, J=13.6, 8.0 Hz, 1H), 2.06-1.99 (m, 1H), 1.86 (dtt, J=12.1, 8.0, 3.2 Hz, 1H), 1.66 (ddt, J=16.0, 12.8, 8.0 Hz, 1H), 1.53 (dd, J=14.7, 8.8 Hz, 1H), 1.41 (dt, J=14.5, 7.1 Hz, 2H), 1.26-1.18 (m, 1H), 0.61 (s, 3H); ³¹P NMR (243 MHz, CDCl₃) δ 149.86; MS (ESI), 890.27 [M−H]⁻.

### Synthesis of 9-((2R,4S,5R)-5-((bis(4-methoxyphenyl)(phenyl)methoxy)methyl)-4-hydroxytetrahydrofuran-2-yl)-7,9-dihydro-8H-purin-8-one

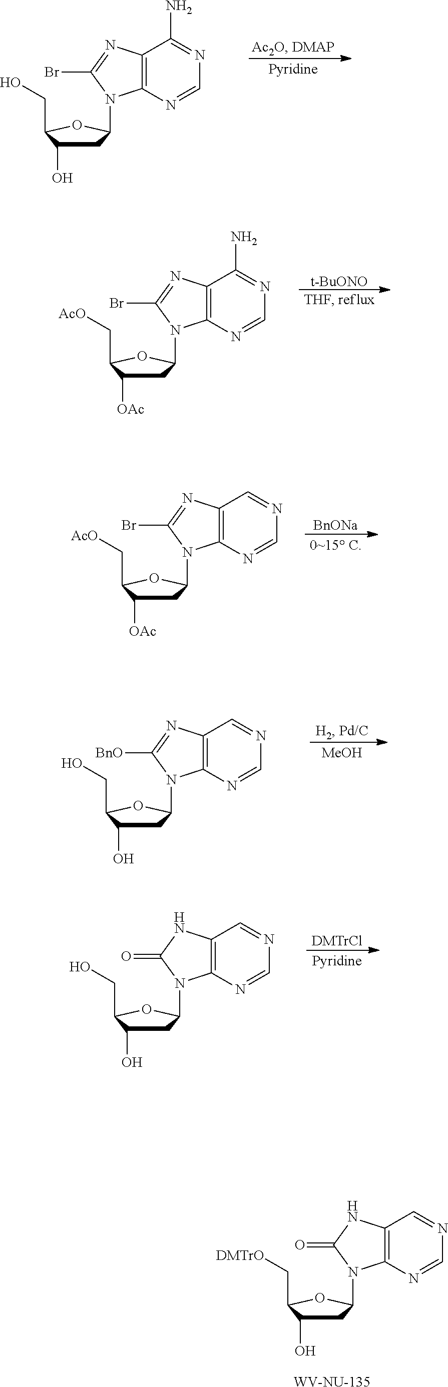

Step 1. To a solution of (2R,3S,5R)-5-(6-amino-8-bromo-9H-purin-9-yl)-2-(hydroxymethyl)tetrahydrofuran-3-ol (50 g, 151.45 mmol, 1 eq.) in pyridine (375 mL) was added Ac₂O (46.38 g, 454.36 mmol, 42.55 mL, 3 eq.) and DMAP (1.85 g, 15.15 mmol, 0.1 eq.). The mixture was stirred at 0-15° C. for 3 hr. TLC (Petroleum ether: Ethyl acetate=0:1, R_{f}=0.29) indicated (2R,3S,5R)-5-(6-amino-8-bromo-9H-purin-9-yl)-2-(hydroxymethyl)tetrahydrofuran-3-ol was consumed and one new spot formed. The reaction mixture was added H₂O (200 mL) and extracted with EtOAc (150 mL*3), dried over Na₂SO₄, filtered and concentrated under reduced pressure to give a residue. The crude ((2R,3S,5R)-3-acetoxy-5-(6-amino-8-bromo-9H-purin-9-yl)tetrahydrofuran-2-yl)methyl acetate (62.7 g, crude) was obtained as a yellow oil. LCMS: (M+H⁺)=414.0, 416.0.

Step 2. To a solution of ((2R,3S,5R)-3-acetoxy-5-(6-amino-8-bromo-9H-purin-9-yl)tetrahydrofuran-2-yl)methyl acetate (26 g, 62.77 mmol, 1 eq.) in THF (520 mL) was added t-BuONO (12.95 g, 125.54 mmol, 14.93 mL, 2 eq.) at 25° C., and stirred at 70° C. for 3 h. then added t-BuONO (12.95 g, 125.54 mmol, 14.93 mL, 2 eq.) to the above reaction mixture and stirred for 3 h. TLC (Petroleum ether: Ethyl acetate=0: 1, R_{f}=0.49) indicated ((2R,3S,5R)-3-acetoxy-5-(6-amino-8-bromo-9H-purin-9-yl)tetrahydrofuran-2-yl)methyl acetate was consumed and three new spots formed. The reaction mixture was concentrated under reduced pressure to remove solvent. The crude product (59.76 g) was purified by column chromatography (SiO₂, Petroleum ether/Ethyl acetate=1/0 to 0/1). TLC: (Petroleum ether: Ethyl acetate=0: 1, R_{f}=0.53). ((2R,3S,5R)-3-acetoxy-5-(8-bromo-9H-purin-9-yl)tetrahydrofuran-2-yl)methyl acetate (28 g, 70.14 mmol, 46.85% yield) was obtained as a yellow solid. LCMS: (M+H⁺)=399.1, 401.

Step 3. To a solution of NaH (22.19 g, 554.85 mmol, 60% purity, 1.5 eq.) in THF (184 mL) was added BnOH (40 g, 369.90 mmol, 38.46 mL, 1 eq.). The mixture was stirred at 0-15° C. for 2 hr. The crude product benzyloxysodium (48 g, crude) as gray liquid was used into the next step without further purification. To a solution of ((2R,3S,5R)-3-acetoxy-5-(8-bromo-9H-purin-9-yl)tetrahydrofuran-2-yl)methyl acetate (17.9 g, 44.84 mmol, 1 eq.) in THF (180 mL) was added BnONa (35.01 g, 269.04 mmol, 32.41 mL, 6 eq.). The mixture was stirred at 15° C. for 12 hr. TLC (Petroleum ether: Ethyl acetate=0:1, R_{f}=0.60) indicated ((2R,3S,5R)-3-acetoxy-5-(8-bromo-9H-purin-9-yl)tetrahydrofuran-2-yl)methyl acetate was consumed and two new spots formed. The reaction mixture was quenched by addition water (300 mL) at 0° C., and extracted with EtOAc (150 mL*3), dried over Na₂SO₄, filtered and concentrated under reduced pressure to give a residue. The residue was purified by column chromatography (SiO₂, Petroleum ether/Ethyl acetate=1/0 to 0/1, then ethyl acetate/methanol=10: 1). (2R,3S,5R)-5-(8-(benzyloxy)-9H-purin-9-yl)-2-(hydroxymethyl)tetrahydrofuran-3-ol (6.4 g, 18.69 mmol, 26.89% yield) was obtained as a yellow solid. LCMS: (M+H⁺)=343.0.

Step 4. To a solution of (2R,3S,5R)-5-(8-(benzyloxy)-9H-purin-9-yl)-2-(hydroxymethyl)tetrahydrofuran-3-ol (5 g, 14.61 mmol, 1 eq.) in MeOH (150 mL) was added Pd/C (2.5 g, 10% purity). The mixture was stirred at 25° C. for 3 hr in H₂ (15 PSI). TLC (Ethyl acetate: Methanol=10:1, R_{f}=0.24) indicated (2R,3S,5R)-5-(8-(benzyloxy)-9H-purin-9-yl)-2-(hydroxymethyl)tetrahydrofuran-3-ol was consumed and one new spot formed. Filtered the Pd/C, the reaction mixture was concentrated under the reduced pressure. 9-((2R,4S,5R)-4-hydroxy-5-(hydroxymethyl)tetrahydrofuran-2-yl)-7,9-dihydro-8H-purin-8-one (3.68 g, crude) was obtained as a yellow oil. LCMS: (M+H⁺)=253.2.

Step 5. To a solution of 9-((2R,4S,5R)-4-hydroxy-5-(hydroxymethyl)tetrahydrofuran-2-yl)-7,9-dihydro-8H-purin-8-one (3.68 g, 14.59 mmol, 1 eq.) in pyridine (72 mL) was added DMTrCl (5.44 g, 16.05 mmol, 1.1 eq.). The mixture was stirred at 15° C. for 12 hr. TLC (Ethyl acetate: Methanol=20:1, R_{f}=0.17) indicated compound 5 was consumed and two new spots formed. The reaction mixture was added H₂O (70 ml) at 0° C., and extracted with EtOAc (50 mL*3), dried over Na₂SO₄, filtered and concentrated under reduced pressure to give a residue. The residue was purified by column chromatography (SiO₂, Petroleum ether/Ethyl acetate=1/1 to 0/1, then ethyl acetate/methanol=10: 1, 5% TEA). Compound WV-NU-135 (3.2 g, 5.77 mmol, 39.55% yield) was obtained as a yellow solid. ¹H NMR (400 MHz, METHANOL-d4) δ=8.38 (s, 1H), 8.21 (s, 1H), 7.44-7.36 (m, 2H), 7.27 (d, J=8.3 Hz, 4H), 7.18 (s, 3H), 6.81-6.69 (m, 4H), 6.37 (s, 1H), 4.74-4.66 (m, 1H), 3.76 (d, J=3.5 Hz, 6H), 3.38-3.33 (m, 1H), 3.29-3.21 (m, 1H), 2.29-2.15 (m, 1H); ¹³C NMR (101 MHz, METHANOL-d4) δ=159.99, 159.93, 154.55, 151.59, 150.98, 146.62, 137.45, 137.43, 134.14, 131.37, 131.27, 129.35, 128.59, 127.66, 123.54, 113.93, 113.90, 87.54, 87.31, 83.08, 73.24, 65.66, 55.71, 49.70, 49.27, 48.85, 49.07 (t, J=42.9 Hz, 1C), 48.43, 36.89, 14.51; LCMS: MS-H⁺=553.3.

### Synthesis of 9-((2R,4S,5R)-5-((bis(4-methoxyphenyl)(phenyl)methoxy)methyl)-4-(((1S,3S,3aS)-3-((methyldiphenylsilyl)methyl)tetrahydro-1H,3H-pyrrolo[1,2-c][1,3,2]oxazaphosphol-1-yl)oxy)tetrahydrofuran-2-yl)-7,9-dihydro-8H-purin-8-one

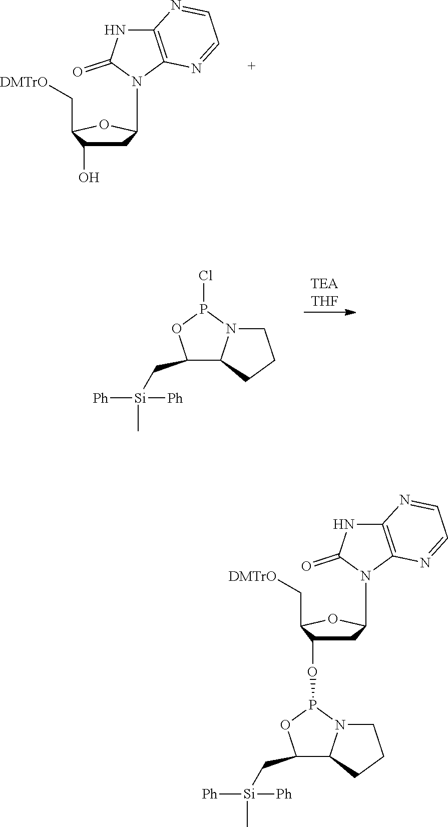

Dry 9-[(2R,4R,5R)-5-[[bis(4-methoxyphenyl)-phenyl-methoxy]methyl]-4-hydroxy-tetrahydrofuran-2-yl]-7H-purin-8-one (1.6 g, 2.89 mmol) in a rbf was dissolved in THF (25 mL). To the clear solution was added triethylamine (2.71 mL, 19.47 mmol). [(3S,3aS)-1-chloro-3a,4,5,6-tetrahydro-3H-pyrrolo[1,2-c][1,3,2]oxazaphosphol-3-yl]methyl-methyl-diphenyl-silane (0.96M solution in THF, 5.42 mL, 5.19 mmol) was added dropwise. The resulting cloudy reaction solution was stirred at rt for 4.5 hr. TLC and LCMS showed the reaction was not complete. Additional triethylamine (0.9 mL, 6.47 mmol) was added followed by additional [(3S,3aS)-1-chloro-3a,4,5,6-tetrahydro-3H-pyrrolo[1,2-c][1,3,2]oxazaphosphol-3-yl]methyl-methyl-diphenyl-silane solution (1.8 mL, 1.72 mmol) dropwise. Stirred for 1 hr. TLC and LCMS showed the reaction was complete. The mixture was filtered, and the filtrate was concentrated. The resulting crude product was purified by normal phase column chromatography applying 0-50% ACN in EtOAc (each mobile phase contained 5% triethylamine) as the gradient to afford 9-((2R,4S,5R)-5-((bis(4-methoxyphenyl)(phenyl)methoxy)methyl)-4-(((1S,3S,3aS)-3-((methyldiphenylsilyl)methyl)tetrahydro-1H,3H-pyrrolo[1,2-c][1,3,2]oxazaphosphol-1-yl)oxy)tetrahydrofuran-2-yl)-7,9-dihydro-8H-purin-8-one as a white foam (1.21 g, 46.8% yield). ¹H NMR (600 MHz, CDCl₃) δ 8.44 (s, 1H), 7.94 (s, 1H), 7.50-7.41 (m, 6H), 7.33-7.27 (m, 7H), 7.19 (dt, J=15.3, 7.0 Hz, 5H), 7.14-7.10 (m, 1H), 6.72 (dd, J=9.0, 2.7 Hz, 4H), 6.29 (t, J=6.9 Hz, 1H), 5.03 (ddt, J=10.0, 7.2, 3.7 Hz, 1H), 4.82 (dt, J=8.9, 5.6 Hz, 1H), 4.02 (q, J=4.7 Hz, 1H), 3.72 (s, 6H), 3.51 (ddt, J=14.6, 10.6, 7.6 Hz, 1H), 3.38 (td, J=11.5, 10.2, 5.6 Hz, 2H), 3.19 (dd, J=10.1, 5.4 Hz, 1H), 3.05-2.94 (m, 2H), 2.10-2.01 (m, 1H), 1.85 (dtd, J=12.6, 8.8, 7.8, 3.5 Hz, 1H), 1.65 (ddd, J=16.4, 12.4, 8.2 Hz, 1H), 1.54 (dd, J=14.6, 9.0 Hz, 1H), 1.41 (dt, J=14.5, 5.4 Hz, 2H), 1.30-1.22 (m, 1H), 0.63 (s, 3H); ³¹P NMR (243 MHz, CDCl₃) δ 149.16; MS (ESI), 892.26 [M−H]⁻.

### Synthesis of 9-((2R,4S,5R)-5-((bis(4-methoxyphenyl)(phenyl)methoxy)methyl)-4-(((1R,3R,3aR)-3-((phenylsulfonyl)methyl)tetrahydro-1H,3H-pyrrolo[1,2-c][1,3,2]oxazaphosphol-1-yl)oxy)tetrahydrofuran-2-yl)-1,9-dihydro-6H-purin-6-one

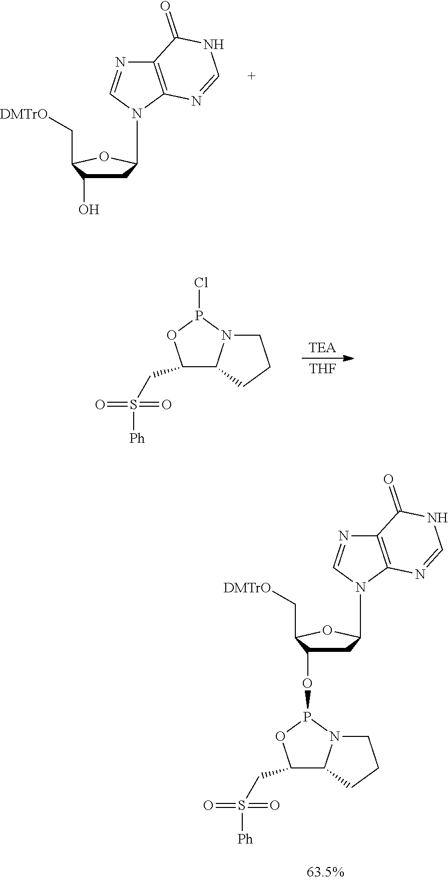

Dry DMT-dI (46.0 g, 82.94 mmol) was partially dissolved in THF (460 mL). To the white fine slurry was added triethylamine (24.28 mL, 174.18 mmol). (3R,3aR)-1-chloro-3-((phenylsulfonyl)methyl)tetrahydro-1H,3H-pyrrolo[1,2-c][1,3,2]oxazaphosphole (0.85M in THF, 156.13 mL, 132.71 mmol) was added slowly. The mixture with some chunk was stirred at rt for 2 hr. Additional THF (230 mL) was added. Continued to stir for 2 hr. The mixture became a white slurry. LCMS showed only minor starting material remained and desired product was major. TLC showed starting material was faint and a less polar spot was major. Stirred for another 2.5 hr. The reaction was quenched by water (1.5 mL). Anhydrous MgSO₄ (20 g) was added. The mixture was filtered through celite, and the clear filtrate was concentrated to afford the crude product as a white foam. The crude product was purified by normal phase column chromatography applying 0-45% anhydrous MeCN in EtOAc as the gradient to afford the title compound as a white foam (44.12 g, 63.5% yield). ¹H NMR (600 MHz, CDCl3) δ 12.78 (s, 1H), 8.01 (s, 1H), 7.99 (s, 1H), 7.94-7.89 (m, 2H), 7.63-7.57 (m, 1H), 7.51 (t, J=7.8 Hz, 2H), 7.42-7.36 (m, 2H), 7.35-7.27 (m, 4H), 7.27-7.23 (m, 2H), 7.23-7.17 (m, 1H), 6.83-6.77 (m, 4H), 6.37 (dd, J=7.2, 6.1 Hz, 1H), 5.10 (ddd, J=7.8, 5.8, 4.5 Hz, 1H), 4.84 (ddt, J=9.1, 6.2, 3.2 Hz, 1H), 4.25 (q, J=4.1 Hz, 1H), 3.77 (s, 6H), 3.68 (dt, J=10.0, 6.1 Hz, 1H), 3.55-3.44 (m, 2H), 3.42-3.33 (m, 3H), 3.20 (tdd, J=10.2, 8.7, 4.0 Hz, 1H), 2.81 (dt, J=13.5, 6.3 Hz, 1H), 2.54 (ddd, J=13.6, 6.1, 3.3 Hz, 1H), 1.92-1.83 (m, 1H), 1.80 (td, J=12.0, 11.2, 6.2 Hz, 1H), 1.68-1.61 (m, 1H), 1.14 (dtd, J=11.8, 10.2, 8.4 Hz, 1H); ³¹P NMR (243 MHz, CDCl₃) δ 156.35; MS (ESI), 836.34 [M−H]⁻.

### Synthesis of (1S,3S,3aS)-1-(((2R,3S)-2-((bis(4-methoxyphenyl)(phenyl)methoxy)methyl)tetrahydrofuran-3-yl)oxy)-3-((phenylsulfonyl)methyl)tetrahydro-1H,3H-pyrrolo[1,2-c][1,3,2]oxazaphosphole

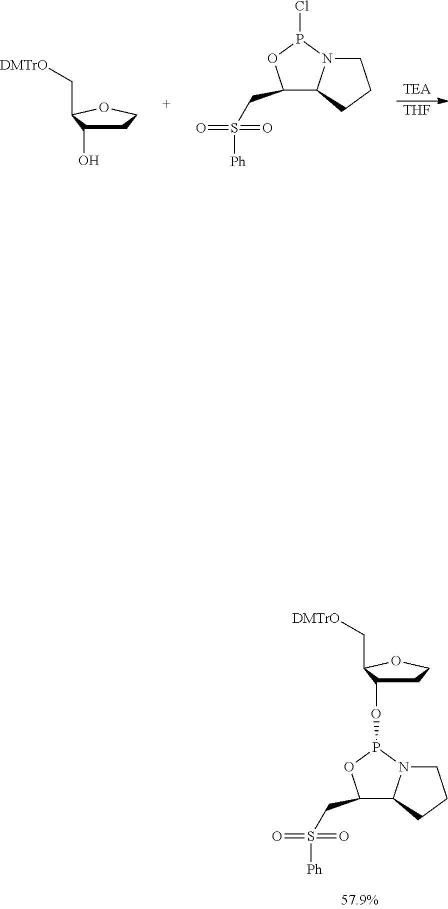

To a white slurry of dry (2R,3R)-2-[[bis(4-methoxyphenyl)-phenyl-methoxy]methyl]tetrahydrofuran-3-ol (15.0 g, 35.67 mmol) in THF (150 mL) was added triethylamine (26.85 mL, 192.63 mmol). (3S,3aS)-3-(benzenesulfonylmethyl)-1-chloro-3a,4,5,6-tetrahydro-3H-pyrrolo[1,2-c][1,3,2]oxazaphosphole (0.9 Min THF, 71.34 mL, 64.21 mmol) was added dropwise. The off-white slurry was stirred at rt for 5 hr. TLC and LCMS showed the reaction was complete. The reaction was quenched by water (642 uL). Anhydrous MgSO₄ (8.56 g) was added. The mixture was filtered through celite, and the filtrate was concentrated to afford the crude product as an off-white greasy foam. The crude product was purified by normal phase column chromatography applying 30-100% DCM in hexanes (each mobile phase contained 2.5% triethylamine) as the gradient to afford the title compound as a yellowish off-white foam (14.53 g, 57.9% yield). ¹H NMR (600 MHz, CDCl₃) δ 7.90-7.86 (m, 2H), 7.58 (tt, J=7.6, 1.2 Hz, 1H), 7.48 (t, J=7.8 Hz, 2H), 7.44 (d, J=7.6 Hz, 2H), 7.35-7.31 (m, 4H), 7.28 (t, J=7.7 Hz, 2H), 7.20 (tt, J=7.3, 1.4 Hz, 1H), 6.85-6.81 (m, 4H), 4.96 (q, J=6.0 Hz, 1H), 4.60 (ddt, J=8.7, 6.1, 2.2 Hz, 1H), 4.03 (td, J=8.2, 3.0 Hz, 1H), 3.94 (ddd, J=9.8, 8.2, 6.2 Hz, 1H), 3.90 (td, J=4.6, 2.2 Hz, 1H), 3.78 (s, 6H), 3.60 (dq, J=10.0, 5.9 Hz, 1H), 3.52-3.43 (m, 2H), 3.35 (dd, J=14.6, 5.4 Hz, 1H), 3.11 (ddd, J=9.1, 5.7, 3.9 Hz, 2H), 3.05 (dd, J=9.8, 4.4 Hz, 1H), 2.16-2.07 (m, 1H), 2.00-1.95 (m, 1H), 1.86 (tt, J=6.1, 2.6 Hz, 1H), 1.76 (ddd, J=18.6, 13.7, 7.7 Hz, 1H), 1.63 (ddt, J=13.4, 7.0, 3.5 Hz, 1H), 1.13-1.05 (m, 1H); ³¹P NMR (243 MHz, CDCl₃) δ 152.40; MS (ESI), 726.86 [M+Na]⁺.

### Synthesis of (2R,3R,4R,5R,6R)-5-acetamido-2-(acetoxymethyl)-6-((5-((6-((2S,4R)-2-((bis(4-methoxyphenyl)(phenyl)methoxy)methyl)-4-(((2-cyanoethoxy)(diisopropylamino)phosphaneyl)oxy)pyrrolidin-1-yl)-6-oxohexyl)amino)-5-oxopentyl)oxy)tetrahydro-2H-pyran-3,4-diyl diacetate

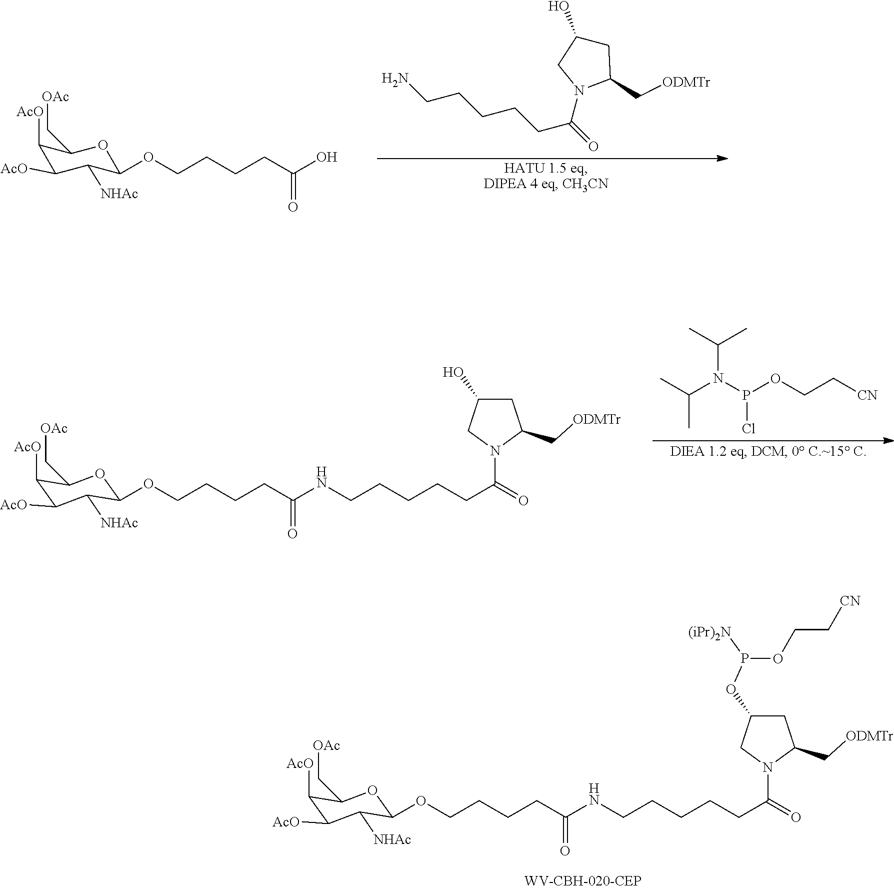

Step 1. For two batches: To a solution of 5-(((2R,3R,4R,5R,6R)-3-acetamido-4,5-diacetoxy-6-(acetoxymethyl)tetrahydro-2H-pyran-2-yl)oxy)pentanoic acid (35 g, 78.22 mmol) in CH₃CN (350 mL) was added HATU (44.61 g, 117.34 mmol) and DIEA (40.44 g, 312.90 mmol), and then added 6-amino-1-((2S,4R)-2-((bis(4-methoxyphenyl)(phenyl)methoxy)methyl)-4-hydroxypyrrolidin-1-yl)hexan-1-one (41.67 g, 78.22 mmol). The mixture was stirred at 15° C. for 12 hr. LCMS showed the main peak was desired. The reaction mixture was added water (500 mL) at 0° C., and extracted with DCM (100 mL *2), dried over Na₂SO₄, filtered and concentrated under reduced pressure to give a residue. The residue was purified by column chromatography (SiO₂, Petroleum ether/Ethyl acetate=1/0 to 0/1, then methanol/ethyl acetate=1: 5, 5% TEA) to get (2R,3R,4R,5R,6R)-5-acetamido-2-(acetoxymethyl)-6-((5-((6-((2S,4R)-2-((bis(4-methoxyphenyl)(phenyl)methoxy)methyl)-4-hydroxypyrrolidin-1-yl)-6-oxohexyl)amino)-5-oxopentyl)oxy)tetrahydro-2H-pyran-3,4-diyl diacetate (122 g, 81.05% yield) as a yellow solid. LCMS: (M−H⁺): 960.5; ¹H NMR (400 MHz, DMSO-d6): δ 7.81 (d, J=9.2 Hz, 1H), 7.69 (t, J=5.6 Hz, 1H), 7.35-7.15 (m, 9H), 6.92-6.83 (m, 4H), 5.75 (s, 4H), 5.21 (d, J=3.2 Hz, 1H), 5.00-4.94 (m, 2H), 4.48 (d, J=8.4 Hz, 1H), 4.43-4.35 (m, 1H), 4.17-3.98 (m, 5H), 3.94-3.77 (m, 1H), 3.73 (s, 7H), 3.64-3.53 (m 1H), 3.49-3.35 (m, 2H), 3.20-3.10 (m, 1H), 3.07-2.93 (m, 3H), 2.20 (t, J=7.2 Hz, 2H), 2.10 (s, 3H), 2.06-1.96 (m, 7H), 1.89 (s, 3H), 1.77 (s, 3H), 1.55-1.33 (m, 8H), 1.31-1.21 (m, 3H).

Step 2. To a solution of (2R,3R,4R,5R,6R)-5-acetamido-2-(acetoxymethyl)-6-((5-((6-((2S,4R)-2-((bis(4-methoxyphenyl)(phenyl)methoxy)methyl)-4-hydroxypyrrolidin-1-yl)-6-oxohexyl)amino)-5-oxopentyl)oxy)tetrahydro-2H-pyran-3,4-diyl diacetate which was dried by toluene (61 g, 63.40 mmol) in DCM (600 mL) was added DIEA (9.83 g, 76.08 mmol) followed by 3-((chloro(diisopropylamino)phosphaneyl)oxy)propanenitrile (15.01 g, 63.40 mmol). The mixture was stirred at 15° C. for 2 hr. The reaction mixture was quenched by addition sat. NaHCO₃ aq. (1000 mL) at 0° C. and extracted with EtOAc (800 mL*3), dried over Na₂SO₄, filtered and concentrated under reduced pressure to give a residue. The residue was purified by column chromatography (SiO₂, Petroleum ether/Ethyl acetate=1/0 to 0/1, then EtOAc/ACN=1/0 to 1/1, 5% TEA) to get (2R,3R,4R,5R,6R)-5-acetamido-2-(acetoxymethyl)-6-((5-((6-((2S,4R)-2-((bis(4-methoxyphenyl)(phenyl)methoxy)methyl)-4-(((2-cyanoethoxy)(diisopropylamino)phosphaneyl)oxy)pyrrolidin-1-yl)-6-oxohexyl)amino)-5-oxopentyl)oxy)tetrahydro-2H-pyran-3,4-diyl diacetate (54 g, 36.64% yield) as a white solid. ¹HNMR (400 MHz, CHLOROFORM-d) δ=7.36-7.27 (m, 2H), 7.25-7.08 (m, 7H), 6.83-6.79 (m, 4H), 6.31-5.98 (m, 2H), 5.29 (d, J=2.9 Hz, 1H), 5.11 (dd, J=3.2, 11.2 Hz, 1H), 4.69-4.58 (m, 1H), 4.53 (s, 1H), 4.34-4.24 (m, 1H), 4.08 (br d, J=4.9 Hz, 1H), 3.82 (br t, J=5.1 Hz, 2H), 3.76-3.65 (m, 7H), 3.57-3.30 (m, 5H), 3.25-2.99 (m, 5H), 2.62-2.48 (m, 2H), 2.33-2.00 (m, 9H), 1.94-1.90 (m, 3H), 1.90-1.82 (m, 3H), 1.76-1.42 (m, 8H), 1.42-1.31 (m, 2H), 1.14-1.02 (m, 12H); ³¹PNMR (162 MHz, CHLOROFORM-d) δ=148.07 (s), 147.93 (s), 147.64 (s), 147.08 (s).

### Example 64. Provided Technologies can Provide Editing in Various Types of Cells

As described herein, provided technologies can provide editing in various types of cells and can be delivered utilizing various delivery technologies (e.g., gymnotic delivery, transfection, etc.). Among other things, the present Example provides results confirming various benefits provided by the provided technologies.

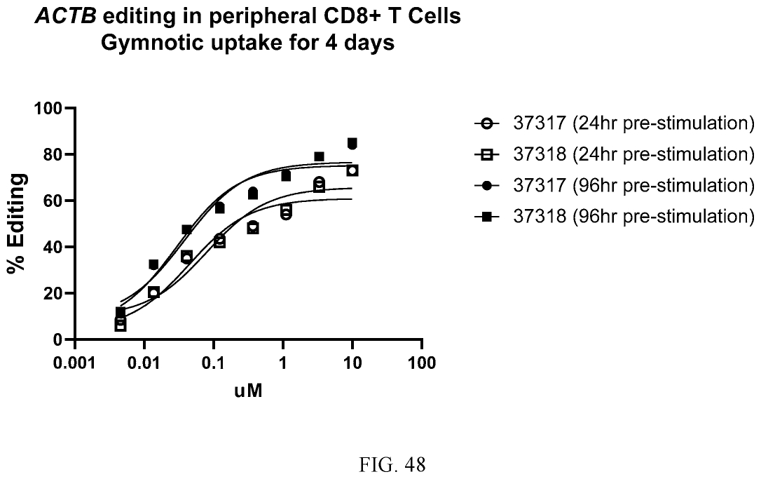

For example, in some instances oligonucleotide compositions (WV-37317 and WV-37318) were delivered by gymnotic delivery to primary human CD8+ T-cells which were pre-stimulated with cytokines for either 24 or 96h. Amount of ACTB mRNA editing was then analyzed 4 days after dosing. As confirmed in FIG. 48, robust editing was achieved at all doses tested. In some embodiments, 96h pre-stimulation showed higher editing then 48h pre-stimulation.

Oligonucleotide compositions were also assessed in multiple fibroblast cell lines by gymnotic delivery or transfection. For example, as shown in FIG. 49, three different primary human fibroblast lines were treated by transfection (50 nM) or by gymnotic uptake (10 uM) with oligonucleotide composition WV-37318 as indicated. RNA was harvested 60 hours later. Editing of target was measured by Sanger sequencing. As confirmed in FIG. 49, ACTB mRNA editing was achieved by both transfection and gymnotic delivery.

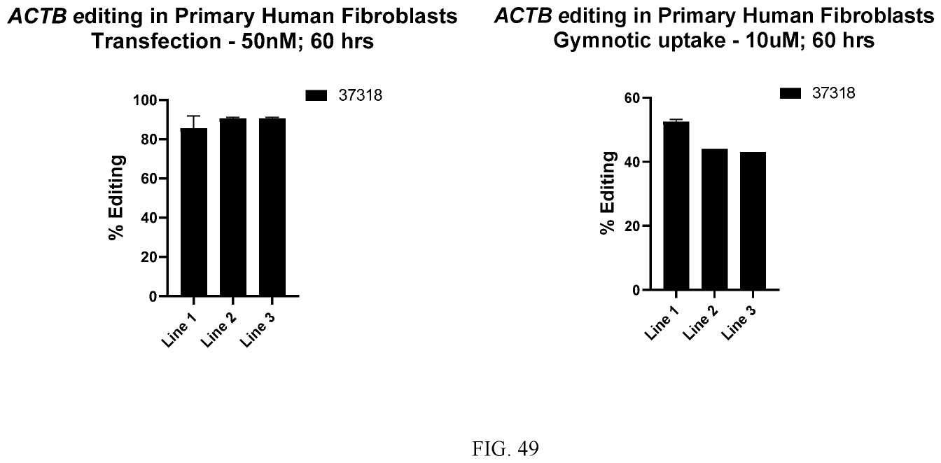

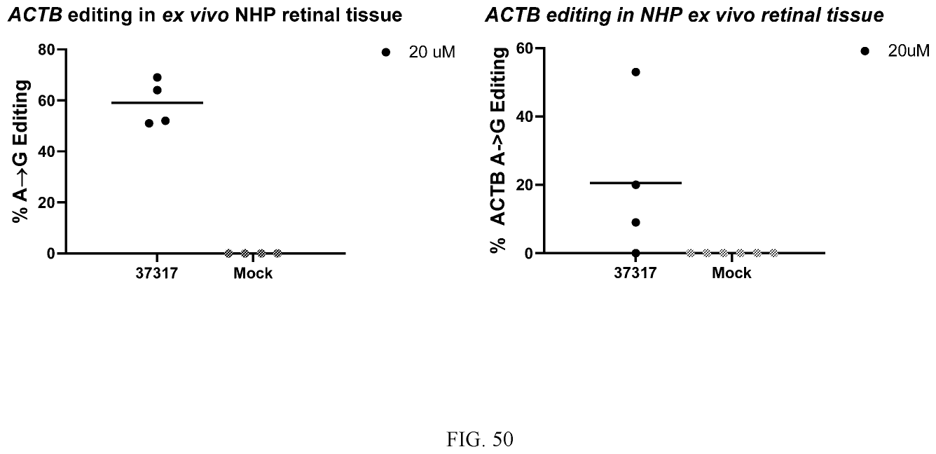

As confirmed in FIG. 50, provided oligonucleotide compositions also provided editing in NHP eyes (retina) ex-vivo. Eyeballs from NHPs were freshly dissected and retinal tissue was treated with oligonucleotide composition WV-37317 by gymnotic uptake. RNA was harvested 48 hours later. Editing of target was measured by Sanger sequencing. As confirmed by FIG. 50, editing was achieved in the NHP eye (Retina) ex-vivo in both sets of experiments.

### Example 65. Various Structural Elements can be Utilized in Accordance with the Present Disclosure to Provide Editing

As described herein, various elements, e.g., sugars, nucleobases, internucleotidic linkages, stereochemistry, additional chemical moieties, etc., can be incorporated into oligonucleotides in accordance with the present disclosure. The present Example, among other things, further confirms that oligonucleotides comprising various elements including various modifications as described in the present disclosure can provide various properties and/or activities.

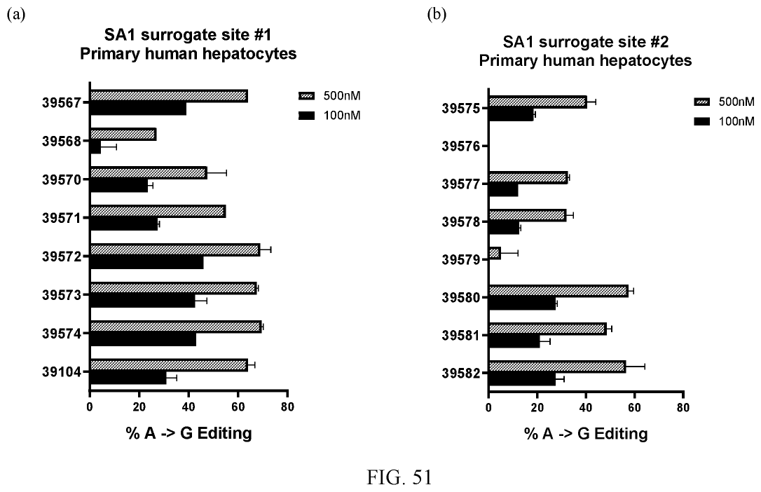

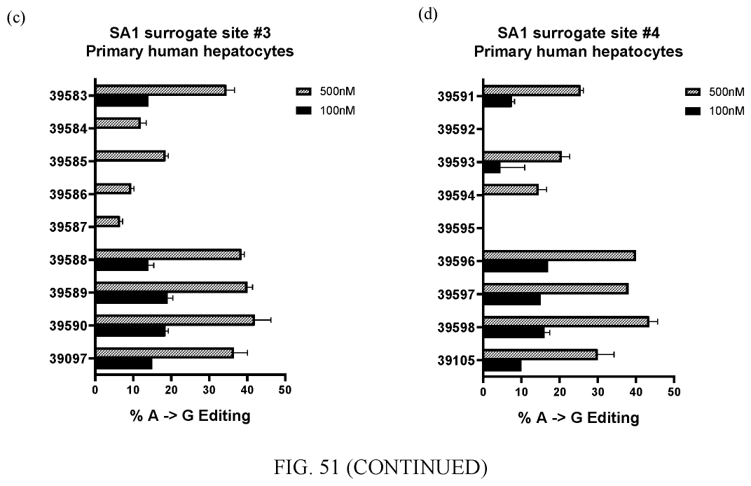

For example, as confirmed by the present Example (e.g., certain data presented in FIG. 51), compositions comprising various features, e.g., modifications, stereochemistry, etc., can provide editing of various adenosines on the SerpinA1 mRNA transcript which selected as targets. These target sites are indicated as surrogate 1 through 4. For each target adenosine, multiple oligonucleotide compositions were designed, prepared and assessed. In some embodiments, certain oligonucleotides differed from each other by one nucleotide. Various modifications were utilized, e.g., 2′-F modified sugars (in first domains and/or second domains (e.g., second and/or third subdomains)), 2′-OR (wherein R is not —H; e.g., 2′-OMe) modified sugars (e.g., in second domains (e.g., second and/or third subdomains)), phosphorothioate internucleotidic linkages (e.g., Sp chirally controlled phosphorothioate internucleotidic linkages (e.g., in first domains and second domains (e.g., in first, second, and/or third subdomains), Rp chirally controlled phosphorothioate internucleotidic linkages (e.g., in second domains (e.g., in first and/or second (e.g., connecting an opposite nucleoside and a 3′ immediate nucleoside) subdomains)), non-negatively charged internucleotidic linkages such as n001 (e.g., in first domains (e.g., as the first internucleotidic linkage linking the first two nucleosides from 5′-end) and/or in second domains (e.g., as the last internucleotidic linkage linking the last two nucleosides from 5′-end and/or as linkage between 3′ immediate nucleosides and their 3′ neighboring nucleosides (e.g., between N₁ and N₂; see, e.g., WV-39588, WV-39590, etc.), etc.))), additional chemical moieties, etc. As confirmed, e.g., in FIG. 51, compositions of oligonucleotides comprising various modifications can provide target editing. Also as confirmed, nucleosides opposite to target adenosines can be placed at various locations in oligonucleotides (e.g., in some cases, positions 5, 6, 7, 8, 9 or more from the 3′-end). Different versions of GalNAc (e.g., in Mod001 or L025) were confirmed to provide delivery and/or activities. As appreciate by those skilled in the art and described and confirmed herein, after editing edited nucleobases may perform various functions of G (and in some instances, editing may be referred to as A to G).

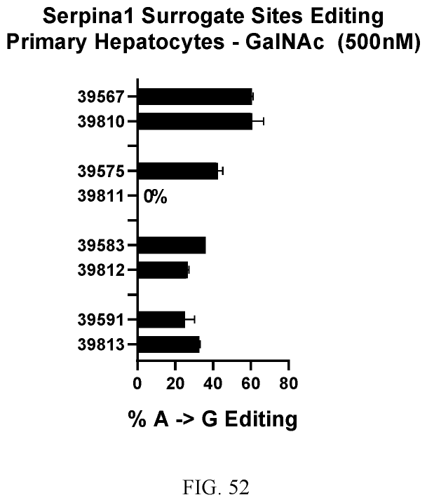

As confirmed in FIG. 52, in various embodiments, natural RNA sugars may be utilized in provided oligonucleotides, and in some cases, in nucleosides opposite to target adenosines. In some embodiments, RNA nucleosides are utilized with 3′ immediate I nucleosides. In some embodiments, a 3′ immediate I nucleoside is bonded to its 3′ immediate nucleoside through Sp non-negatively charged internucleotidic linkages such as n001.

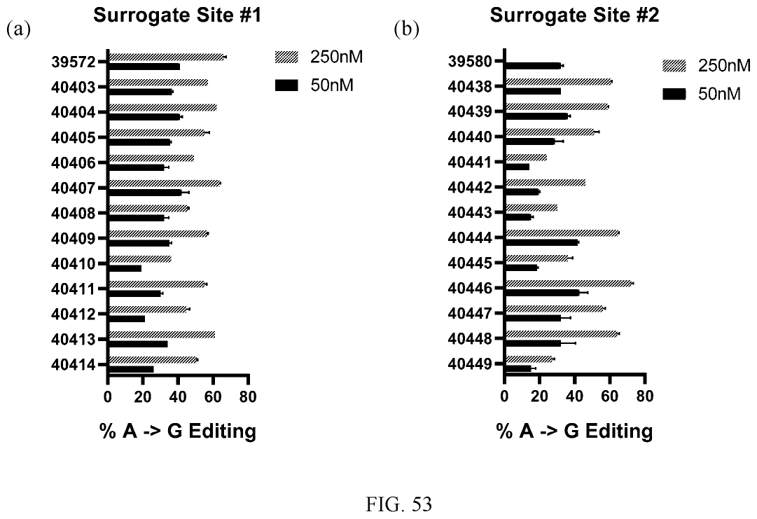

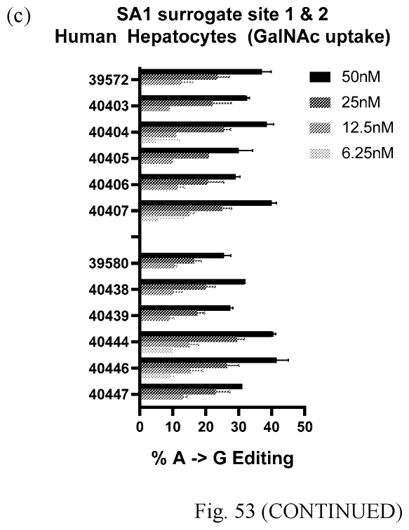

Among other things, FIG. 53 confirms that various number of non-negatively charged internucleotidic linkages may be utilized at various portions in accordance with the present disclosure. Oligonucleotide compositions were dosed in primary human hepatocytes (50 nM and 250 nM) and lower concentrations (50 nM, 25 nM, 12.5 nM, 6.25 nM; see (c)). In some embodiments, certain patterns of modifications (e.g., non-negatively charged internucleotidic linkages and/or stereochemistry thereof) may provide higher efficiencies than others. FIG. 53 also confirms that as described herein, in some embodiments oligonucleotides of various lengths (e.g., 30, 31, 32, etc.) may be utilized for editing.

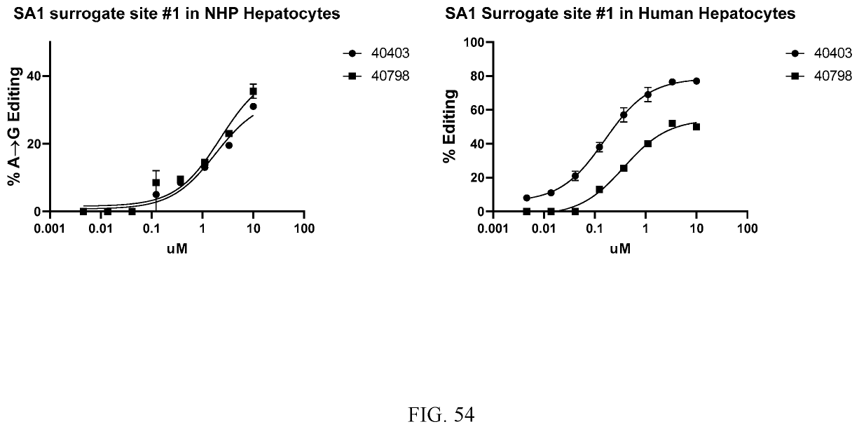

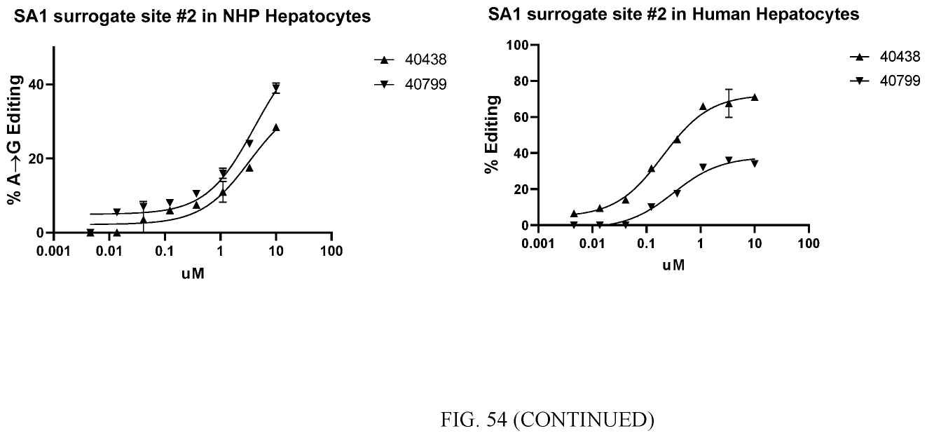

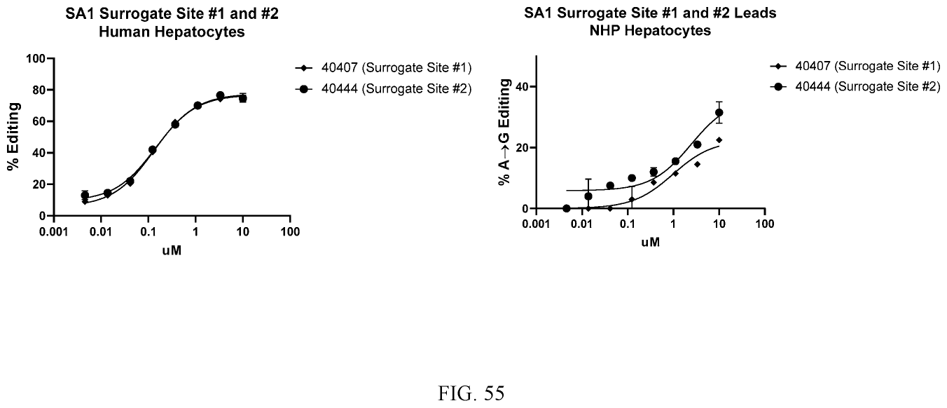

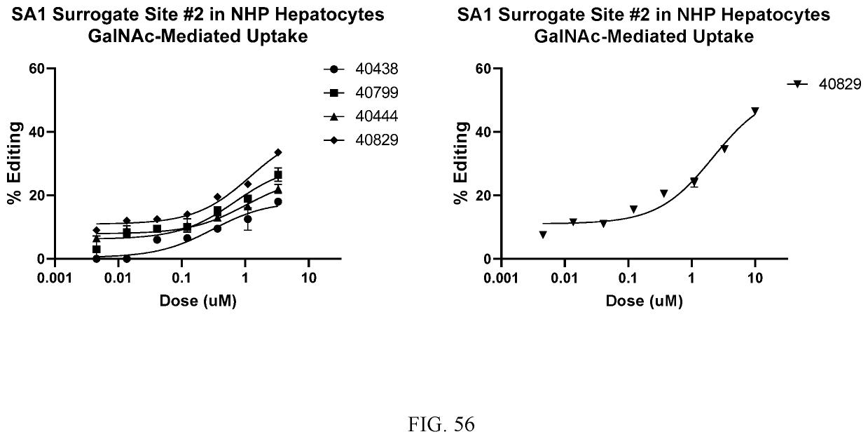

In some embodiments, as confirmed in FIG. 54, removing non-complementary base pairing (e.g., wobbles and/or mismatches) may improve editing efficiency. Oligonucleotides were dosed in primary human and NHP hepatocytes at the indicated dose concentrations. As demonstrated in FIG. 54, in some embodiments, removing wobble (e.g., in the first domain) improved editing levels of NHP surrogate site 1 and 2. In some embodiments, addition of a mismatch decreased editing levels in human serpinA1 surrogate sites 1 and 2. In some embodiments, it was observed that oligonucleotides targeting different surrogate sites, e.g., 1 and 2, may provide similar or the same editing levels at one or more concentrations in human primary hepatocytes while they provide more different editing levels in NHP primary hepatocytes for the corresponding target sites. Among other things, FIG. 56 confirms that in some embodiments, removal of non-complementary bases, addition of more non-negatively charged internucleotidic linkages (e.g., n001; in domain 1 and/or domain 2), and/or more 2′-F modified sugars (e.g., in domain 2) may increase editing levels.

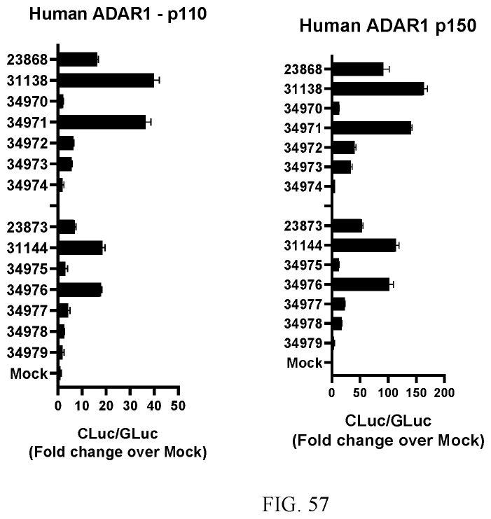

As described herein, various nucleobases including modified nucleobases may be utilized, e.g., at a position across from a target site, in accordance with the present disclosure. In some embodiments, certain nucleobases provide improved properties and/or activities. As an example, data in FIG. 57 confirmed that in some embodiments oligonucleotides comprising various modified nucleobases (or abasic nucleoside) can provide editing. In some embodiments, it was observed that oligonucleotides comprising certain base modifications, such as b001A and b008U, increased editing activity when compared to a reference composition.

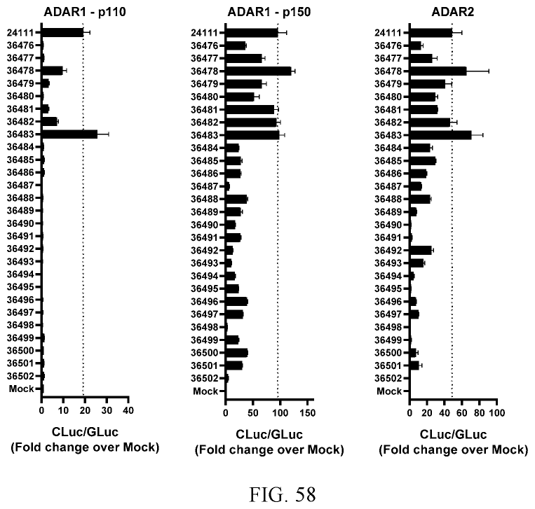

In some embodiments, provided oligonucleotides comprise abasic moieties between nucleosides comprising nucleobases. As confirmed in FIG. 58, various oligonucleotides comprising one or more abasic units in place of nucleosides comprising nucleobases can provide editing activities. A set of oligonucleotides was designed to target the UAG on a cLUC mRNA. In some embodiments, it was observed that abasic units at certain positions provided higher activities than other positions. In some embodiments, it was observed that oligonucleotides may provide different absolute and/or relative editing levels with ADAR1-p110, ADAR1-p150 and ADAR2 in certain circumstances.

FIG. 59 provides additional example data confirming that various base modifications provided herein may be utilized, e.g., in second domains such as positions opposite to target sites, to provide editing activities. Oligonucleotide compositions that target a PIZ mutation in a human SerpinA1 mRNA were designed and contained different base modifications. These oligonucleotide compositions were then transfected into ARPE cells that expressed the human SerpinA1-PIZ allele. In some embodiments, it was observed that certain modified nucleobases b001A and b002A provided higher editing levels. In some embodiments, it was observed that removal of mismatches improved editing levels.

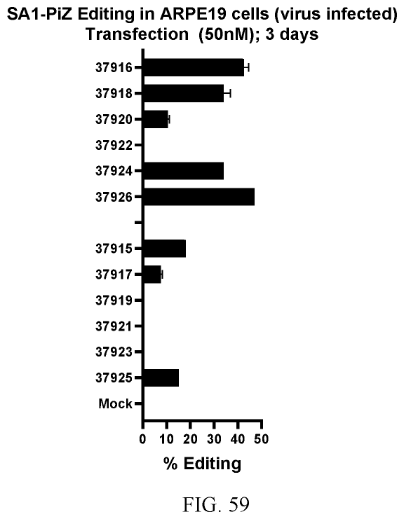

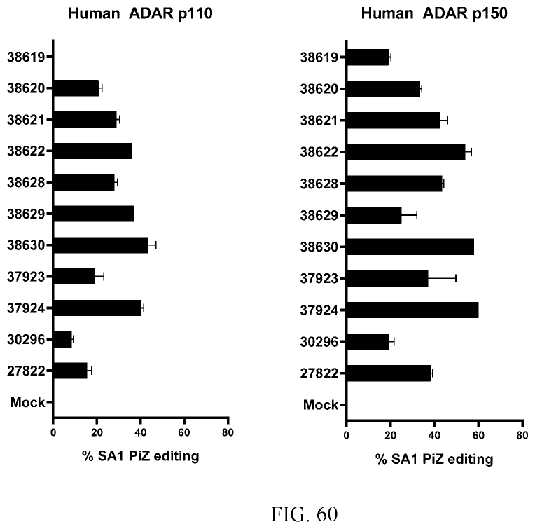

As described herein, provided oligonucleotides comprising various nucleobases (e.g., modified nucleobases) and sugars (e.g., natural DNA or RNA sugars) at positions opposite to target sites can provide editing activities. Certain data confirming editing are presented in FIG. 60. As an example, SerpinA1-PIZ targeting oligonucleotide compositions were transfected into 293T cells that expressed the human SerpinA1-PIZ allele and either ADAR1-p110 or ADAR1-p150. As confirmed in FIG. 60, all oligonucleotide compositions provided SerpinA1-PIZ editing with ADAR1-p110 and/or ADAR1-p150. In some embodiments, it was observed that oligonucleotide compositions that comprise certain modified base, e.g., b001A, provided high editing levels for both human ADAR1-p110 and ADAR1-p150. In some embodiments, it was observed that oligonucleotide compositions that comprise a natural RNA sugar in a nucleoside opposite to a target adenosine provided high editing levels for both human ADAR1-p110 and ADAR1-p150. In some embodiments, it was observed that oligonucleotide compositions of oligonucleotides whose base sequences are fully complementary provided improved editing levels.

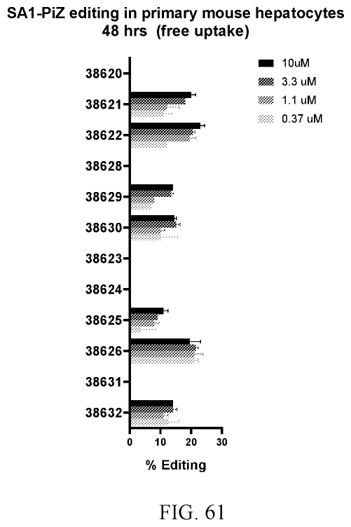

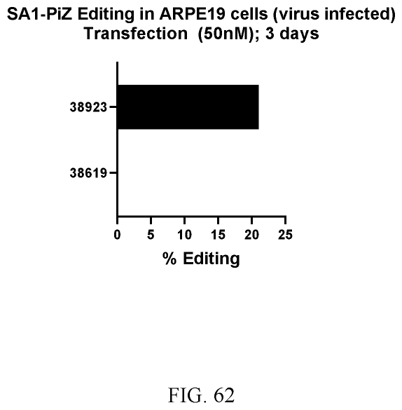

Additional data were presented in FIG. 61 and FIG. 62, confirming that provided oligonucleotides can provide editing through various delivery technologies. Certain SerpinA1-PIZ targeting oligonucleotide compositions were delivered through gymnotic or GalNAc mediated uptake to mouse primary hepatocytes that express a human SerpinA1-PIZ allele at indicated dose concentrations. As confirmed in FIG. 61 SerpinA1-PIZ editing was achieved by both gymnotic and GalNac mediated uptake. In some embodiments, it was observed that compositions of oligonucleotides comprising certain modified nucleobases, e.g., b001A, at sites opposite to target adenosine, can provide improved editing. In some embodiments, it was observed that compositions of oligonucleotides comprising natural RNA sugars, e.g., at sites opposite to target adenosine, can provide improved editing. In some embodiments, it was observed that compositions of oligonucleotides comprising certain modified nucleobases, e.g., b008U, at sites opposite to target adenosine, can provide improved editing.

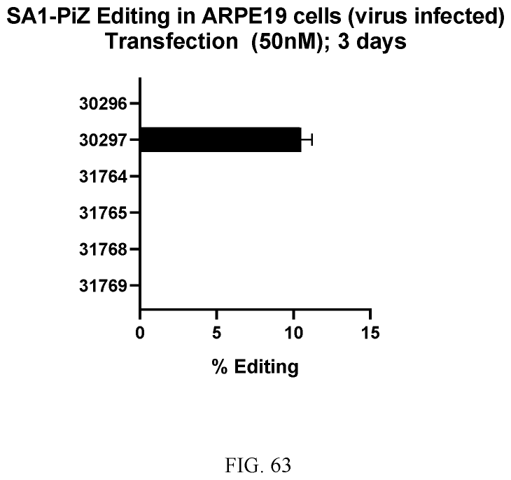

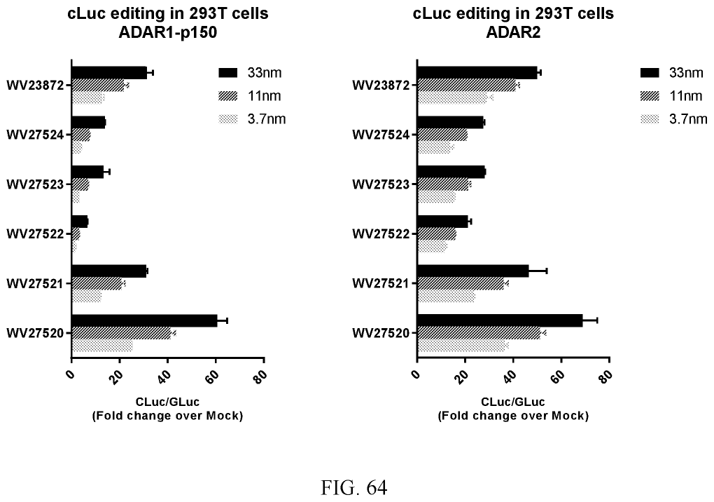

As another example, FIG. 63 confirms that inosine, e.g., in domain 2 of provided oligonucleotides (e.g., as the 3′ immediate nucleoside of a nucleoside opposite to a target nucleoside) can provide editing. In some embodiments, it was observed that a provided oligonucleotide without mismatches can provide higher editing levels. Without the intention to be limited by theory, it is noted that mismatches may impact editing by relatively shorter oligonucleotides more than longer oligonucleotides. Under the specific circumstances of FIG. 63, no significant editing was observed for the other oligonucleotide compositions except WV-30297.

Among other things, FIG. 65 provides additional data confirming that various nucleobases and sugars can be utilized in oligonucleotides in accordance with the present disclosure to provide activities such as editing. In FIG. 65, oligonucleotide compositions target the UAG in a cLUC mRNA. In some embodiments, thymine is replaced with zdnp. As confirmed in FIG. 65, oligonucleotides comprising various nucleobases (e.g., C, zdnp, T, U, etc.) and various sugars (e.g., natural RNA sugars, natural DNA sugars) at sites opposite to target sites, and various sugars at other locations (e.g., 2′-F modified sugars in first domains and/or second domains (e.g., 5′ immediate nucleoside), 2′-OMe modified sugars in 2^{nd} domains, natural DNA sugars in 2^{nd} domains, etc.) can provide editing. In some embodiments, it was observed that a natural RNA sugar at a site opposite to a target site can provide improved editing. In some embodiments, it was observed that oligonucleotides comprising zdnp at sites opposite to target sites can provide improved editing than T.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, configurations, etc. described herein are meant to be example and that actual parameters, dimensions, materials, and/or configurations, etc. will depend upon the specific application or applications for which the teachings of the present disclosure is/are used. Those skilled in the art will recognize, or be able to ascertain using no more than routine experimentation, many equivalents to the specific embodiments described in the present disclosure. It is, therefore, to be understood that the foregoing embodiments are presented by way of example only and that, within the scope of appended claims and equivalents thereto, claimed technologies may be practiced otherwise than as specifically described and claimed. In addition, any combination of two or more features, systems, articles, materials, kits, and/or methods, etc., if such features, systems, articles, materials, kits, and/or methods, etc., are not mutually inconsistent, is included within the scope of the present disclosure.

1-81. (canceled)

82. An oligonucleotide comprising a nucleobase having the structure of

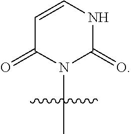 [SMILES: *n1c(=O)cc[nH]c1=O]

83. The oligonucleotide of claim 82, wherein the oligonucleotide has a length of about 10-200 nucleobases.

84. The oligonucleotide of claim 82, wherein the oligonucleotide comprises one or more phosphorothioate linkages.

85. The oligonucleotide of claim 84, wherein each phosphorothioate linkage is independently chirally controlled.

86. The oligonucleotide of claim 82, wherein the oligonucleotide comprises one or more non-negatively charged linkages.

87. The oligonucleotide of claim 82, wherein the oligonucleotide comprises one or more internucleotidic linkages each independently comprising a guanidine moiety.

88. The oligonucleotide of claim 82, wherein the oligonucleotide comprises one or more internucleotidic linkages having the structure of

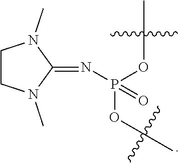 [SMILES: *OP(=O)(N=C1N(C)CCN1C)O*]

89. The oligonucleotide of claim 87, wherein each chiral internucleotidic linkage is independently chirally controlled.

90. The oligonucleotide of claim 82, wherein the oligonucleotide comprises one or more modified sugars.

91. The oligonucleotide of claim 90, wherein the oligonucleotide comprises one or more 2′-F modified sugars.

92. The oligonucleotide of claim 90, wherein the oligonucleotide comprises one or more natural DNA sugars.

93. The oligonucleotide of claim 82, wherein the oligonucleotide comprises a ligand moiety.

94. The oligonucleotide of claim 93, wherein the ligand moiety is a N-acetylgalactosamine (GalNAc) derivative.

95. A pharmaceutical composition which comprises or delivers an effective amount of an oligonucleotide of claim 82 or a pharmaceutically acceptable salt thereof and a pharmaceutically acceptable carrier.

96. An oligonucleotide composition comprising a plurality of oligonucleotides which are of a particular oligonucleotide type characterized by:
a) a common base sequence;
b) a common pattern of backbone linkages;
c) a common pattern of backbone chiral centers;
d) a common pattern of backbone phosphorus modifications;
which composition is chirally controlled in that it is enriched, relative to a substantially racemic preparation of oligonucleotides having the same common base sequence, pattern of backbone linkages and pattern of backbone phosphorus modifications, for oligonucleotides of the particular oligonucleotide type, or a non-random level of all oligonucleotides in the composition that share the common base sequence are oligonucleotides of the plurality; and
wherein each oligonucleotide of the plurality is independently an oligonucleotide of claim 82 or an acid, base, or salt form thereof,
optionally wherein the level of oligonucleotides of a plurality in oligonucleotides in the composition that share the common base sequence of the plurality is about or at least about (DS)^{nc}, wherein DS is about 85%-100% and nc is the number of chirally controlled internucleotidic linkages.

97. A phosphoramidite, wherein the nucleobase of the phosphoramidite is

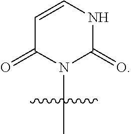 [SMILES: *n1c(=O)cc[nH]c1=O]

98. A phosphoramidite, wherein the phosphoramidite has the structure of R^{NS}—P(OR)N(R)₂, wherein R^{NS} is a optionally protected nucleoside moiety comprising 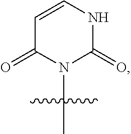 [SMILES: *n1c(=O)cc[nH]c1=O]
and wherein:
each R is independently —H, or an optionally substituted group selected from C₁₋₂₀ aliphatic, C₁₋₂₀ heteroaliphatic having 1-10 heteroatoms, C₆₋₂₀ aryl, C₆₋₂₀ arylaliphatic, C₆₋₂₀ arylheteroaliphatic having 1-10 heteroatoms, 5-20 membered heteroaryl having 1-10 heteroatoms, and 3-20 membered heterocyclyl having 1-10 heteroatoms; or
two R groups are optionally and independently taken together to form a covalent bond; or
two or more R groups on the same atom are optionally and independently taken together with the atom to form an optionally substituted, 3-20 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms;
optionally wherein the phosphoramidite has the structure of R^{NS}—P(OCH₂CH₂CN)N(i-Pr)₂ or wherein the phosphoramidite has the structure of

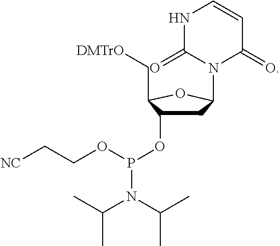

99. The phosphoramidite of claim 98,
wherein the phosphoramidite comprises a chiral auxiliary moiety, wherein the phosphorus is bonded to an oxygen and a nitrogen atom of the chiral auxiliary moiety; or
wherein the phosphoramidite has the structure of: 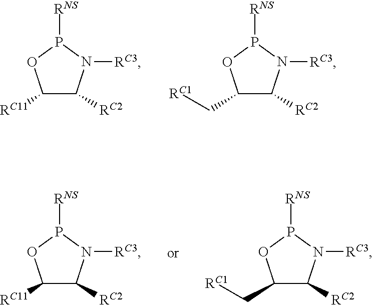
or a salt thereof, wherein R^{NS} is a optionally protected nucleoside moiety comprising 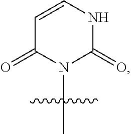 [SMILES: *n1c(=O)cc[nH]c1=O]
and wherein:
R^{C1} is R, —Si(R)₃ or —SO₂R;
R^{C2} and R^{C3} are taken together with their intervening atoms to form an optionally substituted 3-7 membered saturated ring having, in addition to the nitrogen atom, 0-2 heteroatoms, wherein the coupling forms an internucleotidic linkage;
R^{C11} is -L^{C1}-R^{C1}; and
L^{C1} is optionally substituted —CH₂—; or
wherein the phosphoramidite has the structure of 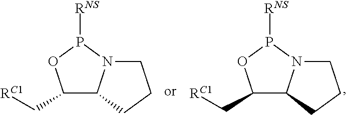
wherein R^{NS} is a optionally protected nucleoside moiety comprising 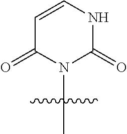 [SMILES: *n1c(=O)cc[nH]c1=O]
and R^{C1} is R, —Si(R)₃ or —SO₂R; and wherein:
each R is independently —H, or an optionally substituted group selected from C1-20 aliphatic, C1-20 heteroaliphatic having 1-10 heteroatoms, C6-20 aryl, C6-20 arylaliphatic, C6-20 arylheteroaliphatic having 1-10 heteroatoms, 5-20 membered heteroaryl having 1-10 heteroatoms, and 3-20 membered heterocyclyl having 1-10 heteroatoms; or
two R groups are optionally and independently taken together to form a covalent bond; or
two or more R groups on the same atom are optionally and independently taken together with the atom to form an optionally substituted, 3-20 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

100. The phosphoramidite of claim 99, wherein R^{C1} is —SiPh₂Me, or
wherein R^{C1} is —SO₂R, wherein R is optionally substituted C₁₋₁₀ aliphatic or wherein R is optionally substituted phenyl, or
wherein the phosphoramidite has the structure of

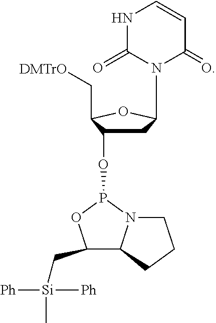

101. A method for preparing an oligonucleotide or a composition, comprising coupling a 5′-OH of an oligonucleotide or a nucleoside with a phosphoramidite of claim 97.

102. A method for modifying a target adenosine in a target nucleic acid, comprising contacting the target nucleic acid with an oligonucleotide of claim 82, optionally wherein the target nucleic acid is or comprises RNA.
